(19) United States
(12) Patent Application Publication
(10) Pub. No.: US 2024/0156960 A1
(43) Pub. Date: May 16, 2024

(54) SYSTEMS AND METHODS FOR EXPRESSING BIOMOLECULES IN A SUBJECT

(71) Applicant: DNARx, San Francisco, CA (US)

(72) Inventors: Robert DEBS, San Francisco, CA (US); Chakkrapong HANDUMRONGKUL, Richmond, VA (US); Timothy HEATH, Madison, WI (US); Alice YE, San Francisco, CA (US)

(73) Assignee: DNARx, San Francisco, CA (US)

(21) Appl. No.: 18/166,962

(22) Filed: Feb. 9, 2023

Related U.S. Application Data
(63) Continuation of application No. PCT/US2021/052040, filed on Sep. 24, 2021.
(60) Provisional application No. 63/083,625, filed on Sep. 25, 2020.

(51) Int. Cl. A61K 39/42; A61K 31/53; A61K 31/573; A61K 39/00; A61K 45/06; A61P 37/04

(52) U.S. Cl. CPC A61K 39/42; A61K 31/53; A61K 31/573; A61K 39/0012; A61K 45/06; A61P 37/04; A61K 2039/505; A61K 2039/53

(57) ABSTRACT

The present invention provides compositions, systems, kits, and methods for expressing at least one therapeutic protein or biologically active nucleic acid molecule in a subject. In certain embodiments, the subject is first administered a composition comprising polycationic structures that is free, or essentially free, of nucleic acid molecules, and then (e.g., 1-30 minutes later) is administered a composition comprising a plurality of one or more non-viral expression vectors that encode at least one therapeutic protein (e.g., at least one anti-SARS-CoV-2 antibody, multiple different antibodies, an ACE2 protein, or human growth hormone) or a biologically active nucleic acid molecule.

The present application is a continuation application of International Patent Application No. PCT/US2021/052040, filed Sep. 24, 2021, which claims the benefit of U.S. Provisional Application No. 63/083,625, filed Sep. 25, 2020, the disclosure of each of which is herein incorporated by reference in its entirety.

### SEQUENCE LISTING

This application contains a computer readable Sequence Listing which has been submitted in XML file format via Patent Center, the entire content of which is incorporated by reference herein in its entirety. The Sequence Listing XML file submitted via Patent Center is entitled “14755-006-999_SEQ_LISTING.xml”, was created on Jan. 24, 2023, and is 4,197,897 bytes in size.

### FIELD OF THE INVENTION

The present invention provides compositions, systems, kits, and methods for expressing at least one therapeutic protein or biologically active nucleic acid molecule in a subject. In certain embodiments, the subject is first administered a composition comprising polycationic structures that is free, or essentially free, of nucleic acid molecules, and then (e.g., 1-30 minutes later) is administered a composition comprising a plurality of one or more non-viral expression vectors that encode at least one therapeutic protein (e.g., at least one anti-SARS-CoV-2 antibody, multiple different antibodies, one anti-SARS-CoV-2 recombinant ACE2 protein, at least one cytokine, or human growth hormone) or a biologically active nucleic acid molecule.

### BACKGROUND

The simplest non-viral gene delivery system uses naked expression vector DNA. Direct injection of free DNA into certain tissues, particularly muscle, has been shown to produce high levels of gene expression, and the simplicity of this approach has led to its adoption in a number of clinical protocols. In particular, this approach has been applied to the gene therapy of cancer where the DNA can be injected either directly into the tumor or can be injected into muscle cells in order to express tumor antigens that might function as a cancer vaccine.

Although direct injection of plasmid DNA has been shown to lead to gene expression, the overall level of expression is much lower than with either viral or liposomal vectors. Naked DNA is also generally thought to be unsuitable for systemic administration due to the presence of serum nucleases. As a result, direct injection of plasmid DNA appears to be limited to only a few applications involving tissues that are easily accessible to direct injection such as skin and muscle cells.

### SUMMARY OF THE INVENTION

The present invention provides compositions, systems, kits, and methods for expressing at least one therapeutic protein or biologically active nucleic acid molecule in a subject. In certain embodiments, the subject is first administered a composition comprising polycationic structures that is free, or essentially free, of nucleic acid molecules, and then (e.g., 1-30 minutes later) is administered a composition comprising a plurality of one or more non-viral expression vectors that encode at least one therapeutic protein (e.g., at least one anti-SARS-CoV-2 antibody, multiple different antibodies, or human growth hormone) or a biologically active nucleic acid molecule. In some embodiments, an agent is further administered (e.g., EPA or DHA) that increases the level and/or length of expression in a subject. In particular embodiments, the first and/or second composition is administered via the subject's airway.

In some embodiments, provided herein are methods comprising: a) administering a first composition to a subject, wherein the first composition comprises polycationic structures, and wherein the first composition is free, or essentially free, of nucleic acid molecules; and b) administering a second composition to the subject after administering the first composition, wherein the second composition comprises a plurality of one or more non-viral expression vectors that encode at least one anti-SARS-CoV-2 antibody or antigen-binding portion thereof, and/or recombinant ACE2, and wherein, as a result of the administering the first and second compositions, the at least one anti-SARS-CoV-2 antibody, or antigen-binding portion thereof, and/or said recombinant ACE2, is expressed in the subject.

In certain embodiments, provided herein are systems comprising: a) a first container; b) a first composition inside the first container and comprising polycationic structures, wherein the first composition is free, or essentially free, of nucleic acid molecules; c) a second container; and d) a second composition inside the second container and comprising a plurality of one or more non-viral expression vectors that encode at least one anti-SARS-CoV-2 antibody or antigen-binding portion thereof, or an ACE2 protein.

In some embodiments, the systems further comprise an Agent that: i) increases the level of expression of the at least one anti-SARS-CoV-2 antibody or antigen-binding portion thereof, or the ACE2 protein, when administered to a subject, and/or ii) and/or the length of time of the expression; as compared to when the agent is not administered to the subject. In further embodiments, the Agent is present in the first composition and/or the second composition. In other embodiments, the systems further comprise: a third container, and wherein the agent is present in the third container.

In certain embodiments, the systems further comprise an anti-viral agent (e.g., Remdesivir or a protein comprising at least part of the ACE2 receptor) and/or an anti-inflammatory and/or anticoagulant.

In particular embodiments, wherein: A) the subject is infected with the SARS-CoV-2 virus, and wherein the at least one anti-SARS-CoV-2 antibody, or antigen-binding portion thereof, or recombinant ACE2 is expressed in the subject at an expression level sufficient to reduce: i) the SARS-CoV-2 viral load in the subject, and/or ii) at least one symptom in the subject caused by the SARS-CoV-2 infection; or B) the subject is not infected with the SARS-CoV-2 virus, and wherein the at least one anti-SARS-CoV-2 antibody, or antigen-binding portion thereof, or recombinant ACE2 is expressed in the subject at an expression level sufficient to prevent the subject from being infected by the SARS-CoV-2 virus.

In certain embodiments, the expression level is maintained in the subject for at least two weeks without: i) any further, or only one, two, or three further repeat, of steps a) and b), and ii) any further administration of vectors encoding the at least one anti-SARS-CoV-2 antibody or antigen-binding portion thereof. In other embodiments, the expression level is maintained in the subject for at least one month without: i) any further, or only one, two, or three further repeat, of steps a) and b), and ii) any further administration of vectors encoding the at least one anti-SARS-CoV-2 antibody or antigen-binding portion thereof. In further embodiments, the expression level is maintained in the subject for at least one year, or two years, or for the lifetime of the subject, without: i) any further, or only one, two, or three further repeat, of steps a) and b), and ii) any further administration of vectors encoding the at least one anti-SARS-CoV-2 antibody or antigen-binding portion thereof. In some embodiments, the at least one anti-SARS-CoV-2 antibody, or antigen-binding portion thereof, is expressed in the subject at a level of: i) between 500 ng/ml and 50 ug/ml, or 10-20 ug/ml, for at least 25 days, or ii) at least 250 ng/ml for at least 25 days.

In some embodiments, provided herein are methods of simultaneously expressing at least three different antibodies, or antigen binding portions thereof, in a subject comprising: a) administering a first composition to a subject, wherein the first composition comprises polycationic structures, and wherein the first composition is free, or essentially free, of nucleic acid molecules; and b) administering a second composition to the subject after administering the first composition, wherein the second composition comprises a plurality of one or more non-viral expression vectors that encode at least three different antibodies or antigen-binding portions thereof, and wherein, as a result of the administering the first and second compositions, the at least three different antibodies, or antigen-binding portions thereof, are simultaneously expressed in the subject. In certain embodiments, the at least three different antibodies, or antigen binding portions thereof, are specific for SARS-CoV-2 and/or influenza A, and/or influenza B. In some embodiments, the at least three different antibodies, or antigen-binding portions thereof, are each fully or substantially neutralizing for SARS-CoV-2. In other embodiments, the at least three different antibodies, or antigen-binding portions thereof, are each fully or substantially neutralizing for a virus selected from the group consisting of: HIV, influenza A, influenza B, and malaria.

In certain embodiments, provided herein are systems comprising: a) a first container; b) a first composition inside the first container and comprising polycationic structures, wherein the first composition is free, or essentially free, of nucleic acid molecules; c) a second container; and d) a second composition inside the second container and comprising a plurality of one or more non-viral expression vectors that encode at least three different antibodies or antigen-binding portions thereof. In certain embodiments, the systems further comprise: an agent that: i) increases the level of expression of at least one of the at least three different antibodies or antigen-binding portions thereof when administered to a subject, and/or ii) and/or the length of time of the expression, as compared to when the agent is not administered to the subject. In other embodiments, the agent is present in the first composition and/or the second composition. In additional embodiments, the systems further comprise a third container, and wherein the agent is present in the third container.

In certain embodiments, the at least three different antibodies or antigen-binding portions thereof, are each expressed in the subject at a level of at least 100 ng/ml (e.g., at least 100 . . . 500 . . . 900 ng/ml). In other embodiments, the at least three different antibodies or antigen-binding portions thereof, are each expressed in the subject at a level of at least 100 ng/ml for at least 25 days. In other embodiments, the at least three different antibodies or antigen-binding portions thereof, are expressed in the subject at a level of at least 200 ng/ml.

In further embodiments, the at least three different antibodies or antigen-binding portions thereof, are expressed in the subject at a level of at least 200 ng/ml for at least 25 days. In other embodiments, wherein: A) the expression level for each of the three different antibodies, or antigen binding portions thereof, is maintained in the subject for at least two weeks, or at least 3 weeks, without: i) any further, or only one further, repeat of steps a) and b), and ii) any further administration of vectors encoding the at least three different antibodies or antigen binding portions thereof; and/or B)repeating steps a) and b) at least once or at least twice. In particular embodiments, the expression level is maintained in the subject for at least two weeks, or at least 3 weeks, without: i) any further, or only one or two further, repeats of steps a) and b), and ii) any further administration of vectors encoding the at least three different antibodies or antigen binding portions thereof.

In other embodiments, the one or more non-viral expression vectors comprise three non-viral expression vectors. In further embodiments, each of the three non-viral expression vector encodes a different antibody or antigen binding fragment thereof. In further embodiments, the one or more non-viral expression vectors comprise six non-viral expression vectors. In additional embodiments, each of the six non-viral expression vectors encodes a different antibody light chain variable region, or heavy chain variable region. In further embodiments, the one or more non-viral expression vectors comprise first, second, and third nucleic acid sequences each encoding an antibody light chain variable region, and fourth, fifth, and sixth nucleic acid sequences each encoding an antibody heavy chain variable region. In other embodiments, the antigen-binding portions thereof are selected from the group consisting of: a Fab′, F(ab)2, Fab, and a minibody.

In some embodiments, at least one of the at least three different antibodies or antigen-binding portions thereof is an anti-SARS-CoV-2 antibody or antigen binding portion thereof. In other embodiments, the at least one of the at least three different antibodies or antigen-binding portions thereof is an antibody or antigen binding portion thereof selected from Table 4 and/or Table 7. In further embodiments, the at least three different antibodies or antigen-binding portions thereof comprise at least four, five, six, seven, or eight different antibodies or antigen-binding portions thereof. In some embodiments, the administering comprises intravenous administering.

In some embodiments, provided herein are methods comprising: a) administering a first composition to a subject, wherein the first composition comprises polycationic structures, and wherein the first composition is free, or essentially free, of nucleic acid molecules; and b) administering a second composition to the subject after administering the first composition, wherein the second composition comprises a plurality of non-viral expression vectors that encode human growth hormone (hGH) and/or hGH linked to a half-life extending peptide (hGH-ext), and wherein, as a result of the administering the first and second compositions, the hGH is expressed in the subject.

In particular embodiments, the hGH and/or hGH-ext is expressed in the subject at a serum expression level of at least 1 ng/ml (e.g., at least 1 . . . 10 . . . 100 . . . 500 ng/ml). In other embodiments, the expression level is maintained in the subject for at least two weeks without: i) any further, or only one further repeat, of steps a) and b), and ii) any further administration of vectors encoding the hGH or hGH-ext. In other embodiments, the expression level is maintained in the subject for at least one month without: i) any further, or only one further repeat, of steps a) and b), and ii) any further administration of vectors encoding the hGH or hGH-ext. In additional embodiments, the expression level is maintained in the subject for at least one year without: i) any further, or only one further repeat, of steps a) and b), and ii) any further administration of vectors encoding the hGH or hGH-ext. In further embodiments, the plurality of non-viral expression vectors encode the hGH-ext, and wherein the half-life extending peptide is selected from the group consisting of: an Fc region peptide, serum albumin, carboxy terminal peptide of human chorionic gonadotropin b-subunit (CTP), and XTEN (see, Schellenberger et al., Nat Biotechnol. 2009 December; 27(12):1186-90). In additional embodiments, the methods further comprise: c) administering an agent, in the first and/or second composition, or present in a third composition, wherein the agent: i) increases the level of expression of the hGH and/or hGH-ext, and/or ii) and/or the length of time of the expression compared to when the agent is not administered to the subject.

In some embodiments, provided herein are systems comprising: a) a first container; b) a first composition inside the first container and comprising polycationic structures, wherein the first composition is free, or essentially free, of nucleic acid molecules; c) a second container; and d) a second composition inside the second container and comprising a plurality of non-viral expression vectors that encode human growth hormone (hGH) and/or hGH linked to a half-life extending peptide (hGH-ext). In certain embodiments, systems further comprise: an Agent that: i) increases the level of expression of the hGH and/or the hGH-ext when administered to a subject, and/or ii) and/or the length of time of the expression; as compared to when the agent is not administered to the subject. In other embodiments, the agent is present in the first composition and/or the second composition. In particular embodiments, the systems further comprise: a third container, and wherein the agent is present in the third container.

In some embodiments, provided herein are methods comprising: a) administering a first composition to a subject, wherein the first composition comprises polycationic structures, and wherein the first composition is free, or essentially free, of nucleic acid molecules; b) administering a second composition to the subject after administering the first composition, wherein the second composition comprises a plurality of expression vectors that each comprise a first nucleic acid sequence encoding a first protein and/or a first biologically active nucleic acid molecule; and c) administering an agent, in the first and/or second composition, or present in a third composition, wherein the agent: i) increases the level of expression of the first protein or the first biologically active nucleic acid molecule, and/or ii) and/or the length of time of the expression; and/or iii) decreases toxicity as measured by alanine aminotransferase (ALT) levels; all as compared to when the agent is not administered to the subject; wherein the agent is selected from the group consisting of: docosahexaenoic acid (DHA), eicosapenaenoic acid (EPA), alpha Linolenic acid (ALA), lipoxin A4 (LA4), 15-deoxy-12,14-prostaglandin J2 (15d), arachidonic acid (AA), cocosapentaenoic acid (DPA), retinoic acid (RA), diallyl disulfide (DADS), oleic acid (OA), alpha tocopherol (AT), sphingosine-1-phosphate (S-1-P), palmitoyl sphingomyelin (SPH), an anti-TNFa antibody or antigen binding fragment thereof, a heparinoid, and N-Acetyl-De-O-Sulfated Heparin; and wherein, as a result of the administering the first and second compositions and the agent to the subject, the first protein or the first biologically active nucleic acid molecule is expressed in the subject.

In other embodiments, the first protein or the first biologically active nucleic acid molecule, is expressed in the subject at a serum expression level of at least 10 ng/ml or at least 100 ng/ml. In additional embodiments, the expression level is maintained in the subject for at least two weeks without: i) any further, or only one further repeat, of steps a), b) and c), and ii) any further administration of vectors encoding the first protein or the first biologically active nucleic acid molecule. In further embodiments, the expression level is maintained in the subject for at least one month without: i) any further, or only one further repeat, of steps a), b) and c), and ii) any further administration of vectors encoding the first protein or the first biologically active nucleic acid molecule. In additional embodiments, the expression level is maintained in the subject for at least one year without: i) any further, or only one further repeat, of steps a), b), and c), and ii) any further administration of vectors encoding the first protein or the first biologically active nucleic acid molecule. In other embodiments, the first nucleic acid sequence provides the first protein or the first biologically active nucleic acid molecule, wherein the first biologically active nucleic acid molecule comprises a sequence selected from: an siRNA or shRNA sequence, a miRNA sequence, an antisense sequence, a CRISPR multimerized single guide, and a CRISPR single guide RNA sequence (sgRNA). In other embodiments, each of the expression vectors further comprises a second nucleic acid sequence encoding: i) a second therapeutic protein, and/or ii) a second biologically active nucleic acid molecule.

In some embodiments, the agent is present in the first composition. In particular embodiments, the agent is present in the third composition, and is administered at least one hour prior to the first composition. In additional embodiments, the agent comprises docosahexaenoic Acid (DHA). In further embodiments, the agent comprises eicosapenaenoic Acid (EPA).

In additional embodiments, provided herein are systems comprising: a) a first container; b) a first composition inside the first container and comprising polycationic structures, wherein the first composition is free, or essentially free, of nucleic acid molecules; c) a second container; and d) a second composition inside the second container and comprising a plurality of expression vectors that each comprise a first nucleic acid sequence encoding a first protein and/or a first biologically active nucleic acid molecule; and e) an agent in the first and/or second composition, or present in a third composition in a third container, wherein the agent is selected from the group consisting of: docosahexaenoic acid (DHA), eicosapenaenoic acid (EPA), alpha Linolenic acid (ALA), lipoxin A4 (LA4), 15-deoxy-12,14-prostaglandin J2 (15d), arachidonic acid (AA), cocosapentaenoic acid (DPA), retinoic acid (RA), diallyl disulfide (DADS), oleic acid (OA), alpha tocopherol (AT), sphingosine-1-phosphate (S-1-P), palmitoyl sphingomyelin (SPH), an anti-TNFa antibody or antigen binding fragment thereof, a heparinoid, and N-Acetyl-De-O-Sulfated Heparin.

In further embodiments, the agent, when administered to a subject with the first and second compositions: i) increases the level of expression of the first protein or the first biologically active nucleic acid molecule, and/or ii) and/or the length of time of the expression; and/or iii) decreases toxicity as measured by alanine aminotransferase (ALT) levels; all as compared to when the agent is not administered to the subject. In other embodiments, the agent is present in the first composition and/or the second composition. In further embodiments, the systems further comprise said third container, and wherein the agent is present in the third container.

In some embodiments, provided herein are methods comprising: a) administering a first composition to a subject via the subject's airway, wherein the first composition comprises polycationic structures, and wherein the first composition is free, or essentially free, of nucleic acid molecules; and b) administering a second composition to the subject after administering the first composition, wherein the administering is via the subject's airway, and wherein the second composition comprises a plurality of expression vectors that each comprise a first nucleic acid sequence encoding a first protein and/or a first biologically active nucleic acid molecule; and wherein, as a result of the administering the first and second compositions to the subject, the first protein or the first biologically active nucleic acid molecule is expressed in the subject.

In certain embodiments, the first protein or the first biologically active nucleic acid molecule is expressed in the subject's lungs. In further embodiments, the first composition is an aqueous composition or a freeze-dried composition. In other embodiments, the second composition is an aqueous composition or a freeze-dried composition. In additional embodiments, the polycationic structure comprise lipids selected from the group consisting of: 1,2-dioleoyl-3-trimethylammonium-propane (DOTAP); 1,2-Dimyristoyl-sn-glycero-3-phosphocholine (DMPC); 1,2-dioleoyl-sn-glycero-3-phospho-L-serine (DOPS); and 1-stearoyl-2-oleoyl-sn-glycero-3-phospho-L-serine. In other embodiments, the subject has lung inflammation. In further embodiments, the subject is on a ventilator.

In additional embodiments, provided herein are systems comprising: a) a first container; b) a first composition inside the first container and comprising polycationic structures, wherein the first composition is free, or essentially free, of nucleic acid molecules, and wherein the polycationic structure comprise lipids selected from the group consisting of: 1,2-dioleoyl-sn-glycero-3-phospho-L-serine (DOPS); and 1-stearoyl-2-oleoyl-sn-glycero-3-phospho-L-serine; c) a second container; and d) a second composition inside the second container and comprising a plurality of expression vectors that each comprise a first nucleic acid sequence encoding a first protein and/or a first biologically active nucleic acid molecule.

In some embodiments, provided herein are systems comprising: a) a first container; b) a first composition inside the first container and comprising polycationic structures, wherein the first composition is free, or essentially free, of nucleic acid molecules; c) a second container; and d) a second composition inside the second container and comprising a plurality of expression vectors that each comprise a first nucleic acid sequence encoding a first protein and/or a first biologically active nucleic acid molecule, wherein the first and/or second composition is a freeze-dried composition.

In some embodiments, provided herein are methods of treating a subject comprising: administering a composition to a subject, wherein the composition comprises: i) an emulsion and/or plurality of liposomes, and ii) an Agent, wherein the subject has: inflammation, an autoimmune disease, an immune-deficiency disease, SARS-CoV-2 infection, and/or is receiving a checkpoint inhibitor, and wherein the Agent selected from the group consisting of: dexamethasone, dexamethasone palmitate, a dexamethasone fatty acid ester, docosahexaenoic Acid (DHA), eicosapenaenoic Acid (EPA), alpha Linolenic Acid (ALA), lipoxin A4 (LA4), 15-deoxy-12,14-Prostaglandin J2 (15d), arachidonic acid (AA), docosapentaenoic acid (DPA), retinoic Acid (RA), diallyl disulfide (DADS), oleic acid (OA), alpha tocopherol (AT), sphingosine-1-phosphate (S-1-P), palmitoyl sphingomyelin (SPH), an anti-TNFa antibody or antigen binding fragment thereof, a heparinoid, and N-Acetyl-De-O-sulfated heparin. In further embodiments, the administration comprises airway administration. In other embodiments, the administration comprises systemic administration. In other embodiments, the composition comprises the liposomes, and wherein Agent is incorporated into the liposomes. In other embodiments, the composition further comprises one or more of the Agents not in the liposomes. In additional embodiments, the composition is free, or essentially free, or nucleic acid molecules. In other embodiments, the subject is infected with SARS-CoV-2, and the method further comprises administering an anti-viral agent to the subject. In further embodiments, the anti-viral agent comprises Remdesivir or a protein comprising at least part of the ACE2 receptor. In other embodiments, the methods further comprise: administering an anti-inflammatory and/or anticoagulant to the subject. In some embodiments, the composition is an aqueous composition or a freeze-dried composition. In additional embodiments, the liposomes comprise lipids selected from the group consisting of: 1,2-dioleoyl-3-trimethylammonium-propane (DOTAP); 1,2-Dimyristoyl-sn-glycero-3-phosphocholine (DMPC); 1,2-dioleoyl-sn-glycero-3-phospho-L-serine (DOPS); and 1-stearoyl-2-oleoyl-sn-glycero-3-phospho-L-serine.

In certain embodiments, provided herein are methods comprising: a) administering a first composition to an animal model, wherein the first composition comprises polycationic structures, and wherein the first composition is free, or essentially free, of nucleic acid molecules, and wherein the animal model is infected with SARS-CoV-2; and b) administering a second composition to the animal model after administering the first composition, wherein the second composition comprises a plurality of one or more non-viral expression vectors that encode first and second anti-SARS-CoV-2 antibodies or antigen-binding portion thereof, and wherein, as a result of the administering the first and second compositions, the first and second candidate anti-SARS-CoV-2 antibodies or antigen-binding portions thereof, are expressed in the animal model; and c) determining the extent to which the expression of the first and second candidate anti-SARS-CoV-2 antibodies, or antigen-binding portions thereof, i) reduce the SARS-CoV-2 viral load in the animal model, and/or ii) reduce at least one symptom in the animal model caused by the SARS-CoV-2 infection. In particular embodiments, the plurality of one or more non-viral expression vectors further encode third, fourth, fifth, sixth, seventh, eighth, ninth, tenth, or eleventh candidate anti-SARS-CoV-2 antibodies or antigen-binding fragments thereof. In certain embodiments, the animal model is selected from a: mouse, rat, hamster, Guinee pig, primate, monkey, chimpanzee, or rabbit. In further embodiments, first and anti-SARS-CoV2 antibodies, or antigen binding portions thereof, are from Table 7 or Table 5. In additional embodiments, the first and second anti-SARS-CoV2 antibodies, or antigen binding portions thereof, are selected from the group consisting of: REGN10933, REGN10987; VIR-7831; LY-CoV1404; LY3853113; Zost 2355K; CV07-209K; C121L; Zost 2504L; CV38-183L; COVA215K; RBD215; CV07-250L; C144L; COVA118L; C135K; and B38. In certain embodiments, the first and second anti-SARS-CoV2 antibodies, or antigen binding portions thereof, are REGN10933 and REGN10987.

In further embodiments, the polycationic structures comprise cationic lipids. In some embodiments, first composition comprises a plurality of liposomes, wherein at least some of said liposomes comprises said cationic lipids. In other embodiments, at least some of said liposomes comprise neutral lipids. In further embodiments, the ratio of said cationic lipids to said neutral lipids in said liposomes is 95:05-80:20 or about 1:1. In other embodiments, the cationic and neutral lipids are selected from the group consisting of: distearoyl phosphatidyl choline (DSPC); hydrogenated or non-hydrogenated soya phosphatidylcholine (HSPC); distearoylphosphatidylethanolamine (DSPE); egg phosphatidylcholine (EPC); 1,2-Distearoyl-sn-glycero-3-phospho-rac-glycerol (DSPG); dimyristoyl phosphatidylcholine (DMPC); 1,2-Dimyristoyl-sn-glycero-3-phosphoglycerol (DMPG); 1,2-Dipalmitoyl-sn-glycero-3-phosphate (DPPA); trimethylammonium propane lipids; DOTIM (1-[2-9(2)-octadecenoylloxy)ethyl]-2-(8(2)-heptadecenyl)-3-(2-hydroxyethyl) midizolinium chloride) lipids; and mixtures of two or more thereof.

In some embodiments, the one or more non-viral expression vectors comprise plasmids, wherein the plasmids are not attached to, or encapsulated in, any delivery agent. In additional embodiments, the one or more non-viral expression vectors comprise a first nucleic acid sequence encoding an antibody light chain variable region, and a second nucleic acid sequence encoding an antibody heavy chain variable region, and optionally, a third nucleic acid sequence encoding an antibody light chain variable region, and a fourth nucleic acid sequence encoding an antibody heavy chain variable region. In certain embodiments, wherein: A) the antigen-binding portion thereof is selected from the group consisting of: a Fab′, F(ab)2, Fab, and a minibody, and/or B) the wherein the at least one anti-SARS-CoV-2 antibody, or antigen-binding portion thereof, is bi-specific for different SARS-CoV-2 antigens. In other embodiments, the anti-SARS-CoV-2 antibody is monoclonal antibody selected from the group consisting of: REGN10933, REGN10987; VIR-7831; LY-CoV1404; LY3853113; Zost 2355K; CV07-209K; C121L; Zost 2504L; CV38-183L; COVA215K; RBD215; CV07-250L; C144L; COVA118L; C135K; and B38. These antibodies are described in the following reference, which are each herein incorporated by reference: Zost et al., Nature Medicine volume 26, pages 1422-1427 (2020); Robbiani et al., Nature volume 584, pages 437-442 (2020); and Wu et al., Science, 2020 Jun. 12;368(6496):1274-1278; and see references in Table 7. Any combination of 2, 3, 4, 5, 6, 7, 8, 9, 10, 11, 12, 13, 14, 15, 16, or all 17 of these antibodies, or antigen binding fragments thereof, may be used in any of the embodiments described herein. In some embodiments, the anti-SARS-CoV-2 antibody, or antigen-binding portion thereof, comprises at least two, three, four, five, six, seven, eight, nine, ten, eleven, twelve, or more of any combination of the following: REGN10933, REGN10987; VIR-7831; LY-CoV1404; LY3853113; Zost 2355K; CV07-209K; C121L; Zost 2504L; CV38-183L; COVA215K; RBD215; CV07-250L; C144L; COVA118L; C135K; and B38 (or any of those shown in Table 7 or Table 5). In additional embodiments, the anti-SARS-CoV-2 antibody, or antigen binding portion thereof, is as described in Table 7.

In some embodiments, the at least one anti-SARS-CoV-2 antibody, or antigen-binding portion thereof, comprises at least two anti-SARS-CoV-2 antibodies, and/or antigen-binding portions thereof, which are expressed in the subject at an expression level sufficient to reduce: i) the SARS-CoV-2 viral load in the subject, and/or ii) at least one symptom in the subject caused by the SARS-CoV-2 infection. In other embodiments, the at least one anti-SARS-CoV-2 antibody, or antigen-binding portion thereof, comprises at least four, or at least eight, or at least eleven, anti-SARS-CoV-2 antibodies and/or antigen-binding portions thereof. In additional embodiments, the at least one anti-SARS-CoV-2 antibody, or antigen-binding portion thereof, comprises at least four, or at least eight, or at least 11, anti-SARS-CoV-2 antibodies and/or antigen-binding portions thereof, and which are expressed in the subject at an expression level sufficient to reduce: i) the SARS-CoV-2 viral load in the subject, and/or ii) at least one symptom in the subject caused by the SARS-CoV-2 infection.

In some embodiments, the administering comprises intravenous administering. In other embodiments, the second composition is administered: i) between 0.5 and 80 minutes after the first composition, or between about 1 and 20 minutes after the first composition. In particular embodiments, the methods further comprise: c) administering an agent, in the first and/or second composition, or present in a third composition, wherein the agent: i) increases the level of expression of the at least one anti-SARS-CoV-2 antibody or antigen-binding portion thereof, and/or ii) and/or the length of time of the expression compared to when the agent is not administered to the subject. In other embodiments, the agent is present in the first composition. In particular embodiments, the agent is present in the third composition, and is administered at least one hour prior to the first composition. In some embodiments, the agent is selected from the group consisting of: dexamethasone, dexamethasone palmitate, a dexamethasone fatty acid ester, Docosahexaenoic Acid (DHA), Eicosapenaenoic Acid (EPA), Alpha Linolenic Acid (ALA), Lipoxin A4 (LA4), 15-deoxy-12,14-Prostaglandin J2 (15d), Arachidonic Acid (AA), Docosapentaenoic Acid (DPA), Retinoic Acid (RA), Diallyl Disulfide (DADS), Oleic Acid (OA), Alpha Tocopherol (AT), Sphingosine-1-Phosphate (S-1-P), Palmitoyl Sphingomyelin (SPH), an anti-TNFa antibody or antigen binding fragment thereof, a heparinoid, and N-Acetyl-De-O-Sulfated Heparin. In certain embodiments, the dexamethasone fatty acid ester has the following Formula:

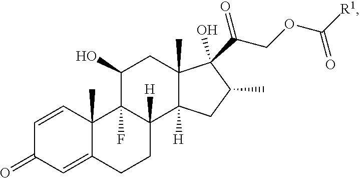

wherein R¹ is C₅-C₂₃ alkyl or C₅-C₂₃ alkenyl.

In certain embodiments, the agent (e.g., water soluble dexamethasone, aka dexamethasone cyclodextrin inclusion complex; see Sigma Sku D2915) is present in the first, second, or third composition at a concentration of 0.1-35 mg/ml or 0.001-1.0 mg/ml (e.g., 0.001 . . . 0.005 . . . 0.01 . . . 0.05 . . . 0.1 . . . 0.5 . . . 1.0 mg/ml). In other embodiments, the subject has lung, cardiovascular, and/or multi-organ inflammation. In particular embodiments, the subject is on a ventilator.

In some embodiments, the first and/or second compositions further comprise a physiologically tolerable buffer or intravenous solution. In other embodiments, the first and/or second compositions further comprise lactated Ringer's solution or saline solution.

In additional embodiments, the first compositions comprise liposomes comprising the polycationic structures, wherein the liposomes further comprising one or more macrophage targeting moieties selected from the group consisting of: mannose moieties, maleimide moieties, a folate receptor ligand, folate, folate receptor antibody or fragment thereof, formyl peptide receptor ligands, N-formyl-Met-Leu-Phe, tetrapeptide Thr-Lys-Pro-Arg, galactose, and lactobionic acid. In other embodiments, the plurality of one or more non-viral expression vectors are not attached to, or encapsulated in, any delivery agent.

In certain embodiments, the subject is a human. In particular embodiments, wherein 0.05-60 mg/mL of the expression vectors are present in the second composition. In other embodiments, the polycationic structures comprise cationic liposomes which are present at a concentration of 0.5-100 mM in the first composition. In further embodiments, the subject is a human, wherein: i) an amount of the first composition is administered such that the human receives a dosage of 2-50 mg/kg of the polycationic structures; and/or ii) an amount of the second composition is administered such that the human receives a dosage of 0.05-60 mg/kg of the expression vectors.

In some embodiments, the polycationic structures comprise cationic liposomes, wherein the cationic liposomes further comprise a lipid bi-layer integrating peptide and/or a target peptide. In certain embodiments, the lipid bi-layer integrating peptide is selected from the group consisting of: surfactant protein D (SPD), surfactant protein C (SPC), surfactant protein B (SPB), and surfactant protein A (SPA), and ii) the target peptide is selected from the group consisting of: microtubule-associated sequence (MTAS), nuclear localization signal (NLS), ER secretion peptide, ER retention peptide, and peroxisome peptide.

In other embodiments, steps a) and b) are repeated between 1 and 60 days after the initial step b). In some embodiments, each of the non-viral expression vectors comprise between 5,500 and 30,000 nucleic acid base pairs. In certain embodiments, the methods further comprise: administering an anti-viral agent to the subject. In some embodiments, the anti-viral agent comprises Remdesivir or a protein comprising at least part of the ACE2 receptor. In additional embodiments, the methods further comprise: administering an anti-inflammatory and/or anticoagulant to the subject. In some embodiments, the one or more non-viral expression vectors are CPG-free or CPG-reduced.

In some embodiments, the Agent herein comprises a dexamethasone fatty acid ester (e.g., as shown in Formula I). For example, dexamethasone palmitate has the following formula

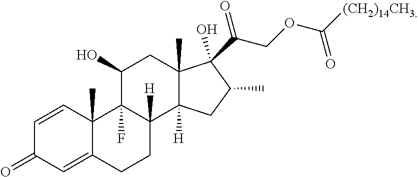

Other fatty acid esters of dexamethasone can also be used, with another fatty acid ester replacing the palmitate group. In some embodiments, the fatty acid ester is a C₆-C₂₄ fatty acid ester, such as hexanoate (caproate), heptanoate (enanthate), octanoate (caprylate), nonanoate (pelargonate), decanoate (caprate), undecanoate, dodecanoate (laurate), tetradecanoate (myristate), octadecenoate (stearate), icosanoate (arachidate), docosanoate (behenate), and tetracosanoate (lignocerate). Accordingly, in some embodiments, the compound is selected from dexamethasone caproate, dexamethasone enanthate, dexamethasone caprylate, dexamethasone pelargonate, dexamethasone caprate, dexamethasone undecanoate, dexamethasone laurate, dexamethasone myristate, dexamethasone palmitate, dexamethasone stearate, dexamethasone arachidate, dexamethasone behenate, and dexamethasone lignocerate.

In certain embodiments, the agent is said dexamethasone fatty acid ester of Formula I, and wherein R¹ is a C₅-C₂₃ alkyl. In other embodiments, the agent is said dexamethasone fatty acid ester of Formula I, and wherein R¹ is a C₅-C₂₃ straight chain alkyl. In other embodiments, the agent is said dexamethasone fatty acid ester of Formula I, and wherein R¹ is a C₁₅ alkyl.

### DESCRIPTION OF THE FIGURES

The patent or application file contains at least one drawing executed in color. Copies of this patent or patent application publication with color drawings will be provided by the Office upon request and payment of the necessary fee.

FIG. 1 shows results from Example 1, showing expression levels over 36 days for four different antibodies or antibody fragments (anti-IL5; 5J8 anti-flu; anti-SARS-CoV-2; and anti-CD20).

FIG. 2 shows results from Example 2, showing expression levels over 43 days for anti-SARS-CoV-2 antibody, as well as expression data for anti-IL5, 5J8 anti-flu, and anti-Sars-Cov2.

FIG. 3A shows results from Example 3, which shows expression levels of multiple unique monoclonal antibodies. FIG. 3B shows results from Example 3, which shows expression levels of the antibodies at various time points over 29 days after initial injection.

FIG. 4 shows results from Example 4, which shows expression levels of antibodies at certain days after injection.

FIG. 5A shows results from Example 5, which shows expression levels of various proteins over 15 days. FIG. 5B shows the results of Example 5, which shows expression levels of various proteins over 22 days.

FIG. 6 shows results from Example 6, which shows expression levels of various antibodies over 22 days.

FIG. 7 shows results from Example 7, which shows expression levels of various proteins.

FIGS. 8A and 8B show results from Example 8, which shows expression levels of cDNA-encoded recombinant ACE2 proteins over 9 days.

FIG. 9 shows results from Example 9 which shows expression levels of human ACE2 and a variant thereof.

FIG. 10 shows the nucleic acid sequence for plasmid 070120 #1: B38-Lambda-BV3 (SEQ ID NO:10).

FIG. 11 shows the nucleic acid sequence for plasmid 070120 #11: B38H-B38L-BV3 Dual (SEQ ID NO:11).

FIG. 12 shows the nucleic acid sequence for plasmid 070320 #4: B38-Kappa-BV3 (SEQ ID NO:12).

FIG. 13 shows the nucleic acid sequence for plasmid 071320 #3: H4-Kappa-BV3 (SEQ ID NO:13).

FIG. 14 shows the nucleic acid sequence for plasmid 080920 #6: H4-H4-Kappa-BV3 (SEQ ID NO:14).

FIG. 15 shows the nucleic acid sequence for plasmid 072620 #5A: 4A8-BV3 (SEQ ID NO:15).

FIG. 16 shows the nucleic acid sequence for plasmid 081820 #2: 4A8-4A8-BV3 (SEQ ID NO:16).

FIG. 17 shows the nucleic acid sequence for plasmid 081820 #3: 4A8-B38Kappa-BV3 (SEQ ID NO:17).

FIG. 18 shows the nucleic acid sequence for plasmid 081820 #4: 4A8-H4-BV3 (SEQ ID NO:18).

FIG. 19 shows the nucleic acid sequence for plasmid 081820 #5: 4A8-shACE2-BV3 (SEQ ID NO:19).

FIG. 20 shows the nucleic acid sequence for plasmid 080420 #3: shACE2-BV3 (SEQ ID NO:20).

FIG. 21 shows the nucleic acid sequence for plasmid 082020 #1: shACE2 TYLTNY-BV3 (SEQ IDNO:21).

FIG. 22 shows the nucleic acid sequence for plasmid 081320 #2A: shACE2-lxL-Fc-BV3 (SEQ ID NO:22).

FIG. 23 shows the nucleic acid sequence for plasmid 081320 #4A: shACE2-1xL-FcLALA-BV3 (SEQ ID NO:23).

FIG. 24 shows the nucleic acid sequence for plasmid 082620 #5A: shACE2 TYLTNY-1xL-FcLALA-BV3 (SEQ ID NO:24).

FIG. 25 shows the nucleic acid sequence for plasmid 080420 #4: shACE2-shACE2-BV3 (SEQ ID NO:25).

FIG. 26 shows the nucleic acid sequence for plasmid 081120 #1: B38Kappa-shACE2-BV3 (SEQ ID NO:26).

FIG. 27 shows the nucleic acid sequence for plasmid 081120 #4: shACE2-B38Kappa-BV3 (SEQ ID NO:27).

FIG. 28 shows the nucleic acid sequence for plasmid 081120 #2: H4-shACE2-BV3 (SEQ ID NO:28).

FIG. 29 shows the nucleic acid sequence of plasmid 081120 #5: shACE2-H4-BV3 (SEQ ID NO:29).

FIG. 30 shows the nucleic acid sequence of plasmid 072320 #2: H4-aCD20-aIL5-5J8-BV2 (SEQ ID NO:30).

FIG. 31 shows the nucleic acid sequence of plasmid 070620 #2: B38 Lambda-aCD20(Cys)-BV3 (SEQ ID NO:31).

FIG. 32 shows the nucleic acid sequence of plasmid 120717 #1: aCD20-aIL5-5J8-BV2 (SEQ ID NO:32).

FIG. 33 shows the nucleic acid sequence of plasmid 122019 #2A: GLA-1xL-hyFc (SEQ ID NO:33).

FIG. 34 shows the nucleic acid sequence of plasmid 011215 #7: hGCSF-BV3 (SEQ ID NO:34).

FIG. 35 shows the nucleic acid sequence of plasmid 071816 #1: (SEQ ID NO:35).

FIG. 36 shows the nucleic acid sequence of plasmid 072520 #4: aCD20-aCD20 (SEQ ID NO:36).

FIG. 37 shows the nucleic acid sequence of plasmid 111517 #1: 5J8-5J8: Double 2A (SEQ ID NO:37).

FIG. 38 shows the nucleic acid sequence of plasmid 111517 #3: aIL5-aIL5: Double 2A (SEQ ID NO:38).

FIG. 39 shows the nucleic acid sequence of plasmid 111517 #19A: 5J8-aIL5: Daul 2A (SEQ ID NO:39).

FIGS. 40A-40F show the nucleic acid sequences of: FIG. 40A) Codon Optimized Human Growth Hormone (hGH1) cDNA (SEQ ID NO:40); FIG. 40B) hGH1-Fc (SEQ ID NO:41); FIG. 40C) Linker GGGGS (SEQ ID NO:42), 1xLinker: GGTGGAGGAGGTAGT (SEQ ID NO:43), 2xLinker: GGTGGAGGAGGTAGTGGGGGTGGAGGTTCA (SEQ ID NO:44), and 3xLinker: GGAGGAGGTGGATCAGGTGGAGGAGGTAGTGGGGGTGGAGGTTCA (SEQ ID NO:45);

FIG. 40D) Fc (SEQ ID NO:46); FIG. 40E) Fc chainA (SEQ ID NO:47); and FIG. 40F) Fc chainB (SEQ ID NO:48).

FIGS. 41A-41F show the nucleic acid sequences of: FIG. 41A) Fc chainAB (SEQ ID NO:49); FIG. 41B) Fc-IgG4 (SEQ ID NO:50); FIG. 41C) hyFc (SEQ ID NO:51); FIG. 41D) mFc (SEQ ID NO:52); FIG. 41E) GAALIE (SEQ ID NO:53); and FIG. 41F) GAALIE-LS (SEQ ID NO:54).

FIGS. 42A-42B show the nucleic acid sequences of: FIG. 42A) hGH1-HSA (SEQ ID NO:55); and FIG. 42B) HSA-K753P-Linker-GH1: (SEQ ID NO:56).

FIGS. 43A-43D show the nucleic acid sequences of: FIG. 43A) hGH1-CTP (SEQ ID NO:57); FIG. 43B) CTP-hGH1-CTP (SEQ ID NO:58); FIG. 43C) CTP-hGH1 (SEQ ID NO:59); and FIG. 43D) XTEN1-hGH1 (SEQ ID NO:60).

FIGS. 44A-44B show the nucleic acid sequences of: FIG. 44A) XTEN1-hGH1-XTEN2 (SEQ ID NO:61); and FIG. 44B) Hgh1-XTEN2 (SEQ ID NO:62).

FIG. 45A shows that expression of the wild type Hgh Cdna fused to a protein half-life extending DNA sequence, including Fc, serum albumin or Xten can significantly increase serum Hgh levels over time in immunocompetent mice. FIG. 45B shows that the Cdna-encoded Hgh protein produced is fully bioactive, as it appropriately increases the levels of the Hgh-regulated, endogenous mouse, IGF-1 protein. FIG. 45C shows one injection of a DNA vector in the procedure of Example 10 procedure drives the wild type Hgh Cdna but lacking any protein half-life extending DNA sequence can produce durable production of therapeutic Hgh serum levels in immunocompetent mice.

FIG. 46 shows that the procedures of Example 11 can be used to express wild type Hgh Cdna fused to a protein half-life extending DNA sequence, including Fc, serum albumin or Xten to significantly increase serum Hgh levels over time in immunocompetent mice.

FIG. 47 shows that, using the procedure of Example 12, one re-injection of a DNA vector driving the wild type Hgh Cdna into fully immunocompetent mice can significantly and durably further increase serum Hgh levels produced by the initial HEDGES Hgh DNA vector injection.

FIG. 48 shows expression levels of hGH fused to an Fc region protein extends the half-life of hGH out to a least 225 days and after a single DNA injection in mice.

FIG. 49 shows expression levels of hGH fused to an Fc region protein out 64 days from treatment.

FIG. 50A shows that selective site-directed mutagenesis of the Fc region of an DNA vector driving the wild type hGH cDNA fused to an Fc protein half-life extending DNA sequence can selectively either increase or decrease serum hGH levels produced in immunocompetent mice.

FIG. 50B shows that selective site-directed mutagenesis of the Fc region of a DNA vector driving the wild type hGH cDNA fused to an Fc protein half-life extending DNA sequence can selectively increase serum hGH levels produced over time in immunocompetent mice.

FIG. 51 shows that incorporating an optimized molar percentage of dexamethasone palmitate (DexPalm) into cationic liposomes can both further increase gene expression and further decrease toxicity.

FIG. 52 shows that incorporating an optimized molar percentage of dexamethasone palmitate into cationic liposomes can both further increase gene expression and further decrease toxicity.

FIG. 53 shows that pre-injecting an optimized molar percentage of dexamethasone palmitate in liposomes prior to injecting cationic liposomes can both further increase gene expression and further decrease toxicity.

FIG. 54 shows that injecting some AILs incorporated into cationic liposomes can both further increase gene expression and further decrease toxicity (ALT levels).

FIG. 55 shows that injecting certain AILs incorporated into cationic liposomes can both further increase gene expression and further decrease toxicity (ALT levels).

FIG. 56 shows that incorporating an optimized molar percentage of dexamethasone palmitate into cationic liposomes can further increase peak levels of gene expression following an otherwise ineffective hG-CSF-DNA dose.

FIG. 57 shows that by selectively modifying the lipid composition of liposomes administered intranasally, that these liposomes can be selectively targeted to intrapulmonary monocytes and macrophages to different extents, thus selectively immune-modulating the lung.

FIG. 58 shows that by selectively modifying a parenteral aqueous soluble pre-dose, and/or the molar percentage of dexamethasone palmitate incorporated into subsequently administered liposomes, that the level of T lymphocyte activation both in lung and in the blood can be selectively immuno-modulated.

FIG. 59 shows that by selectively modifying a parenteral aqueous soluble pre-dose, and/or the molar percentage of dexamethasone palmitate incorporated into subsequently administered liposomes, that the level of T lymphocyte activation both in lung and in the blood can be selectively immuno-modulated.

FIG. 60 shows that pre-administration of an anti-TNF monoclonal antibody, can both further increase gene expression while further reducing its toxicity.

FIG. 61, which shows that either pre- or post-administration of NSH can reduce toxicity.

FIG. 62 shows that either pre- or post-administration of NSH can reduce toxicity.

FIG. 63 shows that either pre-administration of NSH can both further increase gene expression while further reducing its toxicity.

FIG. 64 shows that administration of various formulations of liposomes containing dexamethasone palmitate decreases lymphocyte counts in blood compared to systemic administration of dexamethasone alone.

FIG. 65 shows that administration of various formulations of liposomes containing dexamethasone palmitate decreases monocyte counts in blood compared to systemic administration of dexamethasone alone.

FIG. 66 shows results of Example 22, which shows that one injection of different single DNA expression plasmids each encoding one of five different SARS-CoV2-specific mAb (C135, C215, COV2-2355, CV07-209, and C121) produces fully neutralizing serum levels of each SARS-CoV2-specific mAb for the full experimental course of at least 134 days following administration, and that these ongoing serum mAb levels functionally and continuously block SARS-CoV2 spike-human ACE2 binding for at least 120 days.

FIG. 67 shows results from Example 23, which shows that a single injection results in expression of two SARS-CoV2-specific mAbs from a single plasmid for the course of at least 134 days following this procedure, and that these serum-expressed mAbs sera are functionally capable of blocking SARS-CoV2 spike-human ACE2 interactions for at least 134 days.

FIGS. 68A-68B show results from Example 24 where three different approaches were successfully employed to express simultaneously express two anti-SARS-CoV2 mAbs simultaneously by the three approaches tried. All three approaches successfully allow for the expression of two mAbs in serum of animals at levels (FIG. 68B shows expression levels) that allow for neutralization of SARS-CoV2/ACE2 interactions (FIG. 68B shows neutralization ability).

FIG. 69 shows results from Example 25, which shows that two weekly injections of one or two DNA expression plasmids encoding a total of three different individual SARS-CoV2-specific mAbs produces fully neutralizing serum levels of three different SARS-CoV2-specific mAbs for the course of at least 70 days following administration, and that these ongoing serum mAbs levels functionally and continuously block SARS-CoV2 spike-human ACE2 for at least 70 days.

FIG. 70 shows the results from Example 26, which shows the expression levels and neutralizing ability of four anti-SARS-CoV-2 antibodies expressed in mice.

FIG. 71 shows the results from Example 27, which shows the expression levels and neutralizing ability of four anti-SARS-CoV-2 antibodies expressed in mice.

FIG. 72 shows the results from Example 28, which shows the expression levels and neutralizing ability of four anti-SARS-CoV-2 antibodies expressed in mice.

FIG. 73 shows the results from Example 29, which shows the expression levels and neutralizing ability of four anti-SARS-CoV-2 antibodies expressed in mice.

FIG. 74 shows the results from Example 30, which shows the expression levels and neutralizing ability of four anti-SARS-CoV-2 antibodies expressed in mice.

FIG. 75 shows the results from Example 31, which shows the expression levels and neutralizing ability of five anti-SARS-CoV-2 antibodies expressed in mice.

FIG. 76 shows the results from Example 32, which shows the expression levels and neutralizing ability of six anti-SARS-CoV-2 antibodies expressed in mice.

FIG. 77 shows the results from Example 33, which shows the expression levels and neutralizing ability of six anti-SARS-CoV-2 antibodies expressed in mice.

FIG. 78 shows the results from Example 34, which shows the expression levels and neutralizing ability of six anti-SARS-CoV-2 antibodies expressed in mice.

FIG. 79 shows the results from Example 35, which shows the expression levels and neutralizing ability of eight anti-SARS-CoV-2 antibodies expressed in mice.

FIG. 80 shows the results from Example 36, which shows the expression levels and neutralizing ability of eight anti-SARS-CoV-2 antibodies expressed in mice.

FIG. 81 shows the results from Example 37, which shows the expression levels and neutralizing ability of eight anti-SARS-CoV-2 antibodies expressed in mice.

FIG. 82 shows the results from Example 38, which shows the expression levels and neutralizing ability of eight anti-SARS-CoV-2 antibodies expressed in mice.

FIG. 83 shows the results from Example 39, which shows the expression levels and neutralizing ability of 10 anti-SARS-CoV-2 antibodies, as well as expression levels of other non-Sars-CoV-2 antibodies and various therapeutic proteins, expressed in mice.

FIG. 84 shows the results from Example 40, which shows the expression levels and neutralizing ability of 11 anti-SARS-CoV-2 antibodies, as well as expression levels of other non-Sars-CoV-2 antibodies and various therapeutic proteins, expressed in mice.

FIG. 85 shows the results from Example 41, which shows the expression levels and neutralizing ability of 10 anti-SARS-CoV-2 antibodies, as well as expression levels of other non-Sars-CoV-2 antibodies, expressed in mice.

FIG. 86A shows the results from Example 42, which shows expression levels of the indicated mAbs over 1-48 hours. FIG. 86B shows neutralizing ability of the indicated mAbs over a period of 1-48 hours.

FIG. 87 shows the results from Example 43, which describes the simultaneous expression of six different mAb and genes using a single injection.

FIG. 88 shows the results from Example 44, which describes the use of various eukaryotic promoters to express a target gene (human growth hormone) over 120 days.

FIGS. 89A-89B show the results from Example 45, which describes simultaneously testing 11 different hGLA DNA vectors, showing that they produce a spectrum of serum levels over time.

FIG. 90 shows the results from Example 46, which shows Fc-modified GLA can be expressed in heart tissue at therapeutic levels 104 days after injection of vector.

FIG. 91 shows the results from Example 47, which compares the expression of various mutated Fc regions for GLA-Fc expression.

FIG. 92 shows the results of Example 48, which describes the use of low dose dexamethasone pretreatment does not interfere with the durability of protein expression durability (and acute expression may be augmented).

### DEFINITIONS

As used herein, the phrase “CpG-reduced” refers to a nucleic acid sequence or expression vector that has less CpG di-nucleotides than present in the wild-type versions of the sequence or vector. “CpG-free” means the subject nucleic acid sequence or vector does not have any CpG di-nucleotides. An initial sequence, that contains CpG dinucleotides (e.g., wild-type version of an anti-SARS-CoV-2 antibody), may be modified to remove CpG dinucleotides by altering the nucleic acid sequence. Such CpG di-nucleotides can be suitably reduced or eliminated not just in a coding sequence, but also in the non-coding sequences, including, e.g., 5′ and 3′ untranslated regions (UTRs), promoter, enhancer, polyA, ITRs, introns, and any other sequences present in the nucleic acid molecule or vector. In certain embodiments, the nucleic acid sequences employed herein are CpG-reduced or CpG-free.

As used herein, “empty liposomes” refers to liposomes that do not contain nucleic acid molecules but that may contain other bioactive molecules (e.g., liposomes that are only composed of the lipid molecules themselves, or only lipid molecules and a small molecule drug). In certain embodiments, empty liposomes are used with any of the methods or compositions disclosed herein.

As used herein, “empty cationic micelles” refers to cationic micelles that do not contain nucleic acid molecules but that may contain other bioactive molecules (e.g., micelles that are only composed of lipid and surfactant molecules themselves, or only lipid and surfactant molecules and a small molecule drug). In certain embodiments, empty cationic micelles are used with any of the methods or compositions disclosed herein.

As used herein, “empty cationic emulsions” refers to cationic emulsions or micro-emulsions that do not contain nucleic acid molecules but that may contain other bioactive molecules. In certain embodiments, empty cationic emulsions are used with any of the methods or compositions disclosed herein.

As used herein, the term “alkyl” means a straight or branched saturated hydrocarbon chain containing from 1 to 30 carbon atoms, for example 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈ alkyl), 1 to 6 carbon atoms (C₁-C₆ alkyl), 1 to 4 carbon atoms (C₁-C₄ alkyl), or 5 to 23 carbon atoms (C₅-C₂₃ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “alkenyl” refers to a straight or branched hydrocarbon chain containing from 2 to 30 carbon atoms and containing at least one carbon-carbon double bond, for example 2 to 16 carbon atoms (C₂-C₁₆ alkyl), 2 to 14 carbon atoms (C₂-C₁₄ alkyl), 2 to 12 carbon atoms (C₂-C₁₂ alkyl), 2 to 10 carbon atoms (C₂-C₁₀ alkyl), 2 to 8 carbon atoms (C₂-C₈ alkyl), 2 to 6 carbon atoms (C₂-C₆ alkyl), 2 to 4 carbon atoms (C₂-C₄ alkyl), or 5 to 23 carbon atoms (C₅-C₂₃ alkyl). Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

As used herein, the terms “subject” and “patient” refer to any animal, such as a mammal like a dog, cat, bird, livestock, and preferably a human.

As used herein, the term “administration” refers to the act of giving a composition as described herein to a subject. Exemplary routes of administration to the human body can be through the mouth (oral), skin (transdermal, topical), nose (nasal), lungs (inhalant), oral mucosa (buccal), by injection (e.g., intravenously, subcutaneously, intratumorally, intraocular, intraperitoneally, etc.), and the like.

### DETAILED DESCRIPTION

The present invention provides compositions, systems, kits, and methods for expressing at least one therapeutic protein or biologically active nucleic acid molecule in a subject. In certain embodiments, the subject is first administered a composition comprising polycationic structures that is free, or essentially free, of nucleic acid molecules, and then (e.g., 1-30 minutes later) is administered a composition comprising a plurality of one or more non-viral expression vectors that encode at least one therapeutic protein (e.g., at least one anti-SARS-CoV-2 antibody, multiple different antibodies, at least one recombinant ACE2, or human growth hormone) or a biologically active nucleic acid molecule. In some embodiments, an agent is further administered (e.g., EPA or DHA) that increases the level and/or length of expression in a subject. In particular embodiments, the first and/or second composition is administered via the subject's airway.

The present disclosure provides methods, systems, and compositions, that allow a single injection (e.g., intravenous injection) of cationic liposomes, followed shortly thereafter by injection (e.g., intravenous injection) of vectors encoding at least one protein or biologically active nucleic acid molecule, to produce circulating protein levels many times (e.g., 2-20 times higher) than with other approaches (e.g., allowing for expression for a prolonged period, such at 190 days or over 500 days).

In certain embodiments, the present disclosure employs polycationic structures (e.g., empty cationic liposomes, empty cationic micelles, or empty cationic emulsions) not containing vector DNA, which are administered to a subject prior to vector administration. In certain embodiments, the polycationic structures are cationic lipids and/or are provided as an emulsion. The present disclosure is not limited to the cationic lipids employed, which can be composed, in some embodiments, of one or more of the following: DDAB, dimethyldioctadecyl ammonium bromide; DPTAP (1,2-dipalmitoyl 3-trimethylammonium propane); DHA; prostaglandin, N-[1-(2,3-Dioloyloxy)propyl]-N,N,N-trimethylammonium methylsulfate; 1,2-diacyl-3-trimethylammonium-propanes, (including but not limited to, dioleoyl (DOTAP), dimyristoyl, dipalmitoyl, disearoyl); 1,2-diacyl-3-dimethylammonium-propanes, (including but not limited to, dioleoyl, dimyristoyl, dipalmitoyl, disearoyl) DOTMA, N-[1-[2,3-bis(oleoyloxy)]propyl]-N,N,N-trimethylammoniu-m chloride; DOGS, dioctadecylamidoglycylspermine; DC-cholesterol, 3.beta.-[N-(N′,N′-dimethylaminoethane)carbamoyl]cholesterol; DOSPA, 2,3-dioleoyloxy-N-(2(sperminecarboxamido)-ethyl)-N,N-dimethyl-1-propanami-nium trifluoroacetate; 1,2-diacyl-sn-glycero-3-ethylphosphocholines (including but not limited to dioleoyl (DOEPC), dilauroyl, dimyristoyl, dipalmitoyl, distearoyl, palmitoyl-oleoyl); beta-alanyl cholesterol; CTAB, cetyl trimethyl ammonium bromide; diC14-amidine, N-t-butyl-N′-tetradecyl-3-tetradecylaminopropionamidine; 14Dea2, O,O′-ditetradecanolyl-N-(trimethylammonioacetyl) diethanolamine chloride; DOSPER, 1,3-dioleoyloxy-2-(6-carboxy-spermyl)-propylamide; N,N,N′,N′-tetramethyl-N,N′-bis(2-hydroxylethyl)-2,3-dioleoyloxy-1,4-butan-ediammonium iodide; 1-[2-acyloxy)ethyl]2-alkyl (alkenyl)-3-(2-hydroxyethyl-) imidazolinium chloride derivatives such as 1-[2-(9(Z)-octadecenoyloxy)eth-yl]-2-(8(Z)-heptadecenyl-3-(2-hydroxyethyl)imidazolinium chloride (DOTIM), 1-[2-(hexadecanoyloxy)ethyl]-2-pentadecyl-3-(2-hydroxyethyl)imidazolinium chloride (DPTIM); 1-[2-tetradecanoyloxy)ethyl]-2-tridecyl-3-(2-hydroxyeth-yl)imidazolium chloride (DMTIM) (e.g., as described in Solodin et al. (1995) Biochem. 43:13537-13544, herein incorporated by reference); 2,3-dialkyloxypropyl quaternary ammonium compound derivates, containing a hydroxyalkyl moiety on the quaternary amine, such as 1,2-dioleoyl-3-dimethyl-hydroxyethyl ammonium bromide (DORI); 1,2-dioleyloxypropyl-3-dimethyl-hydroxyethyl ammonium bromide (DORIE); 1,2-dioleyloxypropyl-3-dimethyl-hydroxypropyl ammonium bromide (DORIE-HP), 1,2-dioleyloxypropyl-3-dimethyl-hydroxybutyl ammonium bromide (DORIE-HB); 1,2-dioleyloxypropyl-3-dimethyl-hydroxypentyl ammonium bromide (DORIE-HPe); 1,2-dimyristyloxypropyl-3-dimethyl-hydroxylethyl ammonium bromide (DMRIE); 1,2-dipalmityloxypropyl-3-dimethyl-hydroxyethyl ammonium bromide (DPRIE); 1,2-disteryloxypropyl-3-dimethyl-hydroxyethyl ammonium bromide (DSRIE) (e.g., as described in Felgner et al. (1994) J. Biol. Chem. 269:2550-2561, herein incorporated by reference in its entirety). Many of the above-mentioned lipids are available commercially from, e.g., Avanti Polar Lipids, Inc.; Sigma Chemical Co.; Molecular Probes, Inc.; Northern Lipids, Inc.; Roche Molecular Biochemicals; and Promega Corp.

In certain embodiments, the neutral lipids employed with the methods, compositions, systems, and kits includes diacylglycerophosphorylcholine wherein the acyl chains are generally at least 12 carbons in length (e.g., 12 . . . 14 . . . 20 . . . 24 . . . or more carbons in length), and may contain one or more cis or trans double bonds. Examples of said compounds include, but are not limited to, distearoyl phosphatidyl choline (DSPC), dimyristoyl phosphatidylcholine (DMPC), dipalmitoyl phosphatidylcholine (DPPC), palmitoyl oleoyl phosphatidylcholine (POPC), palmitoyl stearoyl phosphatidylcholine (PSPC), egg phosphatidylcholine (EPC), hydrogenated or non-hydrogenated soya phosphatidylcholine (HSPC), or sunflower phosphatidylcholine.

In certain embodiments, the neutral lipids include, for example, up to 70 mol diacylglycerophosphorylethanolamine/100 mol phospholipid (e.g., 10/100 mol . . . 25/100 mol . . . 50/100 . . . 70/100 mol). In some embodiments, the diacylglycerophosphorylethanolamine has acyl chains that are generally at least 12 carbons in length (e.g., 12 . . . 14 . . . 20 . . . 24 . . . or more carbons in length), and may contain one or more cis or trans double bonds. Examples of such compounds include, but are not limited to distearoylphosphatidylethanolamine (DSPE), dimyristoylphosphatidylethanolamine (DMPE), dipalmitoylphosphatidylethanolamine (DPPE), palmitoyloleoylphosphatidylethanolamine (POPE), egg phosphatidylethanolamine (EPE), and transphosphatidylated phosphatidylethanolamine (t-EPE), which can be generated from various natural or semisynthetic phosphatidylcholines using phospholipase D.

In certain embodiments, the present disclosure employs CpG-reduced or CpG-free expression vectors. An initial sequence that contains CpG dinucleotides (e.g., wild-type version of an anti-SARS-CoV-2 antibody), may be modified to remove CpG dinucleotides by altering the nucleic acid sequence. Such CpG di-nucleotides can be suitably reduced or eliminated not just in a coding sequence, but also in the non-coding sequences, including, e.g., 5′ and 3′ untranslated regions (UTRs), promoter, enhancer, polyA, ITRs, introns, and any other sequences present in the nucleic acid molecule or vector. CpG di-nucleotides may be located within a codon triplet for a selected amino acid. There are five amino acids (serine, proline, threonine, alanine, and arginine) that have one or more codon triplets that contain a CpG di-nucleotide. All five of these amino acids have alternative codons not containing a CpG di-nucleotide that can be changed to, to avoid the CpG but still code for the same amino acid as shown in Table 1 below. Therefore, the CpG di-nucleotides allocated within a codon triplet for a selected amino acid may be changed to a codon triplet for the same amino acid lacking a CpG di-nucleotide.

TABLE 1

| | DNA Codons | DNA Codons |
|---|---|---|
| Amino Acid | Containing CpG | Lacking CpG |
| Serine (Ser or S) | TCG | TCT, TCC, TCA, |
| | | AGT, AGC |
| Proline (Pro or P) | CCG | CCT, CCC, CCA, |
| Threonine (Thr or T) | ACG | ACA, ACT, ACC |
| Alanine (Ala or A) | GCG | GCT, GCC, GCA |
| Arginine (Arg or R) | CGT, CGC, | AGA, AGG |
| | CGA, CGG | |

In addition, within the coding region, the interface between triplets should be taken into consideration. For example, if an amino acid triplet ends in a C-nucleotide which is then followed by an amino acid triplet which can start only with a G-nucleotide (e.g., Valine, Glycine, Glutamic Acid, Alanine, Aspartic Acid), then the triplet for the first amino acid triplet is changed to one which does not end in a C-nucleotide. Methods for making CpG free sequences are shown, for example, in U.S. Pat. No. 7,244,609, which is herein incorporated by reference. A commercial service provided by INVIVOGEN is also available to produce CpG free (or reduced) nucleic acid sequences/vectors (plasmids). A commercial service provided by ThermoScientific produces CpG free nucleotide.

Provided below in Table 2 are exemplary promoters and enhancers that may be used in the vectors described herein. Such promoters, and other promoters known in the art, may be used alone or with any of the enhancers, or enhancers, known in the art. Additionally, when multiple proteins or biologically active nucleic acid molecules (e.g., two, three, four, or more) are expressed from the same vector, the same or different promoters may be used in conjunction with the subject nucleic acid sequence. In some embodiments, a promoter selected from the following list is employed to control the expression levels of the protein or nucleic acid: FerL, FerH, Grp78, hREG1B, and cBOX1. Such promoter can be used, for example, to control production of a protein (e.g., HGH) protein production over a broad temporal range (e.g., without the use of any other modifications including Gene switches).

TABLE 2

| Promoter | Enhancer |
|---|---|
| CMV | human CMV |
| EF1α | mouse CMV |
| Ferritin (Heavy/Light) Chain | SV40 |
| GRP94 | Ubc |
| U1 | AP1 |
| UbC | hr3 |
| Beta Actin | IE2 |
| PGK1 | IE6 |
| GRP78 | E2-RS |
| CAG | MEF2 |
| SV40 | C/EBP |
| TRE | HNF-1 |

In some embodiments, compositions and systems herein are provided and/or administered in doses selected to elicit a therapeutic and/or prophylactic effect in an appropriate subject (e.g., mouse, human, etc.). In some embodiments, a therapeutic dose is provided. In some embodiments, a prophylactic dose is provided. Dosing and administration regimes are tailored by the clinician, or others skilled in the pharmacological arts, based upon well-known pharmacological and therapeutic/prophylactic considerations including, but not limited to, the desired level of pharmacologic effect, the practical level of pharmacologic effect obtainable, toxicity. Generally, it is advisable to follow well-known pharmacological principles for administrating pharmaceutical agents (e.g., it is generally advisable to not change dosages by more than 50% at time and no more than every 3-4 agent half-lives). For compositions that have relatively little or no dose-related toxicity considerations, and where maximum efficacy is desired, doses in excess of the average required dose are not uncommon. This approach to dosing is commonly referred to as the “maximal dose” strategy. In certain embodiments, a dose (e.g., therapeutic of prophylactic) is about 0.01 mg/kg to about 200 mg/kg (e.g., 0.01 mg/kg, 0.02 mg/kg, 0.05 mg/kg, 0.1 mg/kg, 0.2 mg/kg, 0.5 mg/kg, 1.0 mg/kg, 2.0 mg/kg, 5.0 mg/kg, 10 mg/kg, 20 mg/kg, 50 mg/kg, 100 mg/kg, 200 mg/kg, or any ranges therebetween (e.g., 5.0 mg/kg to 100 mg/kg)). In some embodiments, a subject is between 0.1 kg (e.g., mouse) and 150 kg (e.g., human), for example, 0.1 kg, 0.2 kg, 0.5 kg, 1.0 kg, 2.0 kg, 5.0 kg, 10 kg, 20 kg, 50 kg, 100 kg, 200 kg, or any ranges therebetween (e.g., 40-125 kg). In some embodiments, a dose comprises between 0.001 mg and 40,000 mg (e.g., 0.001 mg, 0.002 mg, 0.005 mg, 0.01 mg, 0.02 mg, 0.05 mg, 0.1 kg, 0.2 mg, 0.5 mg, 1.0 mg, 2.0 mg, 5.0 mg, 10 mg, 20 mg, 50 mg, 100 mg, 200 mg, 500 mg, 1,000 mg, 2,000 mg, 5,000 mg, 10,000 mg, 20,000 mg, 40,000 mg, or ranges therebetween.

In certain embodiments, a target peptide is used with the cationic or neutral liposomes in the compositions herein. Exemplary target peptides are shown in Table 3 below. In table 3, “[n]” prefix indicates the N-terminus and a “[c]” suffix indicates the C-terminus; sequences lacking either are found in the middle of the protein.

TABLE 3

| Target | Sequence | Source protein or organism |
|---|---|---|
| nucleus (NLS) | PKKKRKV (SEQ ID NO: 1) | SV40 large T antigen (P03070) |
| Out of nucleus (NES) | IDMLIDLGLDLSD (SEQ ID NO: 2) | HSV transcriptional regulator |
| | | IE63 P10238 |
| ER, secretion (signal | [n]MMSFVSLLLVGILFWATEAEQLTKCEVF | Lactalbumin (P09462) |
| peptide) | Q (SEQ ID NO: 3) | |
| ER, retention (KDEL) | KDEL[c] (SEQ ID NO: 4) | |
| Mitochondrial matrix | [n]MLSLRQSIRFFKPATRTLCSSRYLL | S. cerevisiae COX4 (P04037) |
| | (SEQ ID NO: 5) | |
| Plastid | [n]MVAMAMASLQSSMSSLSLSSNSFLGQ | Pisum sativum RPL24 (P11893) |
| | PLSPITLSPFLQG (SEQ ID NO: 6) | |
| Folded secretion (Tat) | (S/T)RRXFLK (SEQ ID NO: 7) | Near the N terminus[6] |
| peroxisome (PTS1) | SKL[c] (SEQ ID NO: 8) | |
| peroxisome (PTS2) | [c]XXXXRLXXXXXHL (SEQ ID NO: 9) | |

In certain embodiments, one or more (e.g., at least 3, or at least 8 antibodies) are expressed with the systems and methods herein. In some embodiments, this includes the therapeutic monoclonal antibodies (mAbs), Fabs, F(ab)2s, and scFv's that are shown in Table 4 below, as well as the anti-SARS-CoV2 antibodies and antigen bindings provided at Table 5 and Table 7, which is herein incorporated by reference.

TABLE 4

| Antibody Name | Trade name | Type | Source | Target | Use |
|---|---|---|---|---|---|
| 3F8 | | mab | mouse | GD2 ganglioside | neuroblastoma |
| 8H9 | | mab | mouse | B7-H3 | neuroblastoma, |
| | | | | | sarcoma, metastatic |
| | | | | | brain cancers |
| Abagovomab | | mab | mouse | CA-125 (imitation) | ovarian cancer |
| Abciximab | ReoPro | Fab | chimeric | CD41 (integrin | platelet aggregation |
| | | | | alpha-IIb) | inhibitor |
| Abituzumab | | mab | humanized | CD51 | cancer |
| Abrilumab | | mab | human | integrin α4β7 | inflammatory bowel |
| | | | | | disease, ulcerative |
| | | | | | colitis, Crohn's disease |
| Actoxumab | | mab | human | Clostridium | Clostridium difficile |
| | | | | difficile | colitis |
| Adalimumab | Humira | mab | human | TNF-α | Rheumatoid arthritis, |
| | | | | | Crohn's Disease, Plaque |
| | | | | | Psoriasis, Psoriatic |
| | | | | | Arthritis, Ankylosing |
| | | | | | Spondylitis, Juvenile |
| | | | | | Idiopathic Arthritis, |
| | | | | | Hemolytic disease of |
| | | | | | the newborn |
| Adecatumumab | | mab | human | EpCAM | prostate and breast |
| | | | | | cancer |
| Aducanumab | | mab | human | beta-amyloid | Alzheimer's disease |
| Afasevikumab | | mab | human | IL17A and IL17F | — |
| Afelimomab | | F(ab′)₂ | mouse | TNF-α | sepsis |
| Afutuzumab | | mab | humanized | CD20 | lymphoma |
| Alacizumab pegol | | F(ab′)₂ | humanized | VEGFR2 | cancer |
| ALD518 | | — | humanized | IL-6 | rheumatoid arthritis |
| Alemtuzumab | Lemtrada, | mab | humanized | CD52 | Multiple sclerosis |
| | Campath | | | | |
| Alirocumab | | mab | human | PCSK9 | hypercholesterolemia |
| Altumomab pentetate | Hybri-ceaker | mab | mouse | CEA | colorectal cancer |
| | | | | | (diagnosis) |
| Amatuximab | | mab | chimeric | mesothelin | cancer |
| Anatumomab | | Fab | mouse | TAG-72 | non-small cell lung |
| mafenatox | | | | | carcinoma |
| Anetumab ravtansine | | mab | human | MSLN | cancer |
| Anifrolumab | | mab | human | interferon α/β | systemic lupus |
| | | | | receptor | erythematosus |
| Anrukinzumab | | mab | humanized | IL-13 | asthma |
| (=IMA-638) | | | | | |
| Apolizumab | | mab | humanized | HLA-DR — | hematological cancers |
| Arcitumomab | CEA-Scan | Fab′ | mouse | CEA | gastrointestinal cancers |
| | | | | | (diagnosis) |
| Ascrinvacumab | | mab | human | activin receptor- | cancer |
| | | | | like kinase 1 | |
| Aselizumab | | mab | humanized | L-selectin | severely injured patients |
| | | | | (CD62L) | |
| Atezolizumab | | mab | humanized | CD274 | cancer |
| Atinumab | | mab | human | RTN4 | — |
| Atlizumab | Actemra, | mab | humanized | IL-6 receptor | rheumatoid arthritis |
| (=tocilizumab) | RoActemra | | | | |
| Atorolimumab | | mab | human | Rhesus factor | hemolytic disease of the |
| | | | | | newborn[citation |
| | | | | | needed] |
| Avelumab | | mab | human | CD274 | — |
| Bapineuzumab | | mab | humanized | beta amyloid | Alzheimer's disease |
| Basiliximab | Simulect | mab | chimeric | CD25 (α chain of | prevention of organ |
| | | | | IL-2 receptor) | transplant rejections |
| Bavituximab | | mab | chimeric | phosphatidylserine | cancer, viral infections |
| Bectumomab | LymphoScan | Fab | mouse | CD22 | non-Hodgkin's |
| | | | | | lymphoma (detection) |
| Begelomab | | mab | mouse | DPP4 | — |
| Belimumab | Benlysta, | mab | human | BAFF | non-Hodgkin |
| | Lympho Stat- | | | | lymphoma etc. |
| | B | | | | |
| Benralizumab | | mab | humanized | CD125 | asthma |
| Bertilimumab | | mab | human | CCL11 (eotaxin-1) | severe allergic disorders |
| Besilesomab | Scintimun | mab | mouse | CEA-related | inflammatory lesions |
| | | | | antigen | and metastases |
| | | | | | (detection) |
| Bevacizumab | Avastin | mab | humanized | VEGF-A | metastatic cancer, |
| | | | | | retinopathy of |
| | | | | | prematurity |
| Bezlotoxumab | | mab | human | Clostridium | Clostridium difficile |
| | | | | difficile | colitis |
| Biciromab | FibriScint | Fab′ | mouse | fibrin II, beta chain | thromboembolism |
| | | | | | (diagnosis) |
| Bimagrumab | | mab | human | ACVR2B | myostatin inhibitor |
| Bimekizumab | | mab | humanized | IL 17A and IL 17F | — |
| Bivatuzumab | | mab | humanized | CD44 v6 | squamous cell |
| mertansine | | | | | carcinoma |
| Bleselumab | | mab | human | CD40 | — |
| Blinatumomab | | BiTE | mouse | CD19 | pre-B ALL (CD19+) |
| Blontuvetmab | Blontress | mab | veterinary | CD20 | — |
| Blosozumab | | mab | humanized | SOST | osteoporosis |
| Bococizumab | | mab | humanized | neural apoptosis- | dyslipidemia |
| | | | | regulated | |
| | | | | proteinase 1 | |
| Brazikumab | | mab | human | IL23 | Crohn's disease |
| Brentuximab vedotin | | mab | chimeric | CD30 (TNFRSF8) | hematologic cancers |
| Briakinumab | | mab | human | IL-12, IL-23 | psoriasis, rheumatoid |
| | | | | | arthritis, inflammatory |
| | | | | | bowel diseases, |
| | | | | | multiple sclerosis |
| Brodalumab | | mab | human | IL-17 | inflammatory diseases |
| Brolucizumab | | mab | humanized | VEGFA | wet age-related macular |
| | | | | | degeneration |
| Brontictuzumab | | mab | humanized | Notch 1 | cancer |
| Burosumab | | mab | human | FGF 23 | X-linked |
| | | | | | hypophosphatemia |
| Cabiralizumab | | mab | humanized | CSF1R | — |
| Canakinumab | Ilaris | mab | human | IL-1 — | rheumatoid arthritis |
| Cantuzumab | | mab | humanized | mucin CanAg | colorectal cancer etc. |
| mertansine | | | | | |
| Cantuzumab | | mab | humanized | MUC1 | cancers |
| ravtansine | | | | | |
| Caplacizumab | | mab | humanized | VWF | thrombotic |
| | | | | | thrombocytopenic |
| | | | | | purpura, thrombosis |
| Capromab pendetide | Prostascint | mab | mouse | prostatic | prostate cancer |
| | | | | carcinoma cells | (detection) |
| Carlumab | | mab | human | MCP-1 | oncology/immune |
| | | | | | indications |
| Carotuximab | | mab | chimeric | endoglin | — |
| Catumaxomab | Removab | 3funct | rat/mouse | EpCAM, CD3 | ovarian cancer, |
| | | | hybrid | | malignant ascites, |
| | | | | | gastric cancer |
| cBR96-doxorubicin | | mab | humanized | Lewis-Y antigen | cancer |
| immunoconjugate | | | | | |
| Cedelizumab | | mab | humanized | CD4 | prevention of organ |
| | | | | | transplant rejections, |
| | | | | | treatment of |
| | | | | | autoimmune diseases |
| Cergutuzumab | | mab | humanized | IL2 | — |
| amunaleukin | | | | | |
| Certolizumab pegol | Cimzia | Fab′ | humanized | TNF-α | Crohn's disease |
| | | | | | Rheumatoid arthritis |
| | | | | | axial spondyloarthritis |
| | | | | | psoriasis arthritis |
| Cetuximab | Erbitux | mab | chimeric | EGFR | metastatic colorectal |
| | | | | | cancer and head and |
| | | | | | neck cancer |
| Ch.14.18 | | mab | chimeric | GD2 ganglioside | neuroblastoma |
| Citatuzumab bogatox | | Fab | humanized | EpCAM | ovarian cancer and |
| | | | | | other solid tumors |
| Cixutumumab | | mab | human | IGF-1 receptor | solid tumors |
| | | | | (CD221) | |
| Clazakizumab | | mab | humanized | Oryctolagus | rheumatoid arthritis |
| | | | | cuniculus | |
| Clenoliximab | | mab | chimeric | CD4 | rheumatoid arthritis |
| Clivatuzumab | hPAM4-Cide | mab | humanized | MUC1 | pancreatic cancer |
| tetraxetan | | | | | |
| Codrituzumab | | mab | humanized | glypican 3 | cancer |
| Coltuximab ravtansine | | mab | chimeric | CD19 | cancer |
| Conatumumab | | mab | human | TRAIL-R2 | cancer |
| Concizumab | | mab | humanized | TFPI | bleeding |
| CR6261 | | mab | human | Influenza A | infectious |
| | | | | hemagglutinin | disease/influenza A |
| Crenezumab | | mab | humanized | 1-40-β-amyloid | Alzheimer's disease |
| Crotedumab | | mab | human | GCGR | diabetes |
| Dacetuzumab | | mab | humanized | CD40 | hematologic cancers |
| Daclizumab | Zenapax | mab | humanized | CD25 (α chain of | prevention of organ |
| | | | | IL-2 receptor) | transplant rejections |
| Dalotuzumab | | mab | humanized | IGF-1 receptor | cancer etc. |
| | | | | (CD221) | |
| Dapirolizumab pegol | | mab | humanized | CD154 (CD40L) | — |
| Daratumumab | | mab | human | CD38 (cyclic ADP | cancer |
| | | | | ribose hydrolase) | |
| Dectrekumab | | mab | human | IL-13 | — |
| Demcizumab | | mab | humanized | DLL4 | cancer |
| Denintuzumab | | mab | humanized | CD19 | cancer |
| mafodotin | | | | | |
| Denosumab | Prolia | mab | human | RANKL | osteoporosis, bone |
| | | | | | metastases etc. |
| Depatuxizumab | | mab | chimeric/ | EGFR | cancer |
| mafodotin | | | humanized | | |
| Derlotuximab biotin | | mab | chimeric | histone complex | recurrent glioblastoma |
| | | | | | multiforme |
| Detumomab | | mab | mouse | B-lymphoma cell | lymphoma |
| Dinutuximab | | mab | chimeric | GD2 ganglioside | neuroblastoma |
| Diridavumab | | mab | human | hemagglutinin | influenza A |
| Domagrozumab | | mab | humanized | GDF-8 | Duchenne muscular |
| | | | | | dystrophy |
| Dorlimomab aritox | | F(ab′)₂ | mouse | — | — |
| Drozitumab | | mab | human | DR5 | cancer etc. |
| Duligotumab | | mab | human | ERBB3 (HER3) | testicular cancer |
| Dupilumab | | mab | human | IL4 | atopic diseases |
| Durvalumab | | mab | human | CD274 | cancer |
| Dusigitumab | | mab | human | ILGF2 | cancer |
| Ecromeximab | | mab | chimeric | GD3 ganglioside | malignant melanoma |
| Eculizumab | Soliris | mab | humanized | C5 | paroxysmal nocturnal |
| | | | | | hemoglobinuria, |
| | | | | | atypical HUS |
| Edobacomab | | mab | mouse | endotoxin | sepsis caused by Gram- |
| | | | | | negative bacteria |
| Edrecolomab | Panorex | mab | mouse | EpCAM | colorectal carcinoma |
| Efalizumab | Raptiva | mab | humanized | LFA-1 (CD11a) | psoriasis (blocks T-cell |
| | | | | | migration) |
| Efungumab | Mycograb | scFv | human | Hsp90 | invasive Candida |
| | | | | | infection |
| Eldelumab | | mab | human | interferon gamma- | Crohn's disease, |
| | | | | induced protein | ulcerative colitis |
| Elgemtumab | | mab | human | ERBB3 (HER3) | cancer |
| Elotuzumab | | mab | humanized | SLAMF7 | multiple myeloma |
| Elsilimomab | | mab | mouse | IL-6 | — |
| Emactuzumab | | mab | humanized | CSF1R | cancer |
| Emibetuzumab | | mab | humanized | HHGFR | cancer |
| Emicizumab | | mab | humanized | activated F9, F10 | haemophilia A |
| Enavatuzumab | | mab | humanized | TWEAK receptor | cancer etc. |
| Enfortumab vedotin | | mab | human | AGS-22M6 | cancer expressing |
| | | | | | Nectin-4 |
| Enlimomab pegol | | mab | mouse | ICAM-1 (CD54) | — |
| Enoblituzumab | | mab | humanized | CD276 | cancer |
| Enokizumab | | mab | humanized | IL9 | asthma |
| Enoticumab | | mab | human | DLL4 | — |
| Ensituximab | | mab | chimeric | 5AC | cancer |
| Epitumomab cituxetan | | mab | mouse | episialin | — |
| Epratuzumab | | mab | humanized | CD22 | cancer, SLE |
| Erenumab | | mab | human | CGRP | migraine |
| Erlizumab | | F(ab′)₂ | humanized | ITGB2 (CD18) | heart attack, stroke, |
| | | | | | traumatic shock |
| Ertumaxomab | Rexomun | 3funct | rat/mouse | HER2/neu, CD3 | breast cancer etc. |
| | | | hybrid | | |
| Etaracizumab | Abegrin | mab | humanized | integrin αvβ3 | melanoma, prostate |
| | | | | | cancer, ovarian cancer |
| | | | | | etc. |
| Etrolizumab | | mab | humanized | integrin α7 β7 | inflammatory bowel |
| | | | | | disease |
| Evinacumab | | mab | human | angiopoietin 3 | dyslipidemia |
| Evolocumab | | mab | human | PCSK9 | hypercholesterolemia |
| Exbivirumab | | mab | human | hepatitis B surface | hepatitis B |
| | | | | antigen | |
| Fanolesomab | NeutroSpec | mab | mouse | CD15 | appendicitis (diagnosis) |
| Faralimomab | | mab | mouse | interferon receptor | — |
| Farletuzumab | | mab | humanized | folate receptor 1 | ovarian cancer |
| Fasinumab | | mab | human | HNGF | acute sciatic pain |
| FBTA05 | Lymphomun | 3funct | rat/mouse | CD20 | chronic lymphocytic |
| | | | hybrid | | leukaemia |
| Felvizumab | | mab | humanized | respiratory | respiratory syncytial |
| | | | | syncytial virus | virus infection |
| Fezakinumab | | mab | human | IL-22 | rheumatoid arthritis, |
| | | | | | psoriasis |
| Fibatuzumab | | mab | humanized | ephrin receptor A3 | — |
| Ficlatuzumab | | mab | humanized | HGF | cancer etc. |
| Figitumumab | | mab | human | IGF-1 receptor | adrenocortical |
| | | | | (CD221) | carcinoma, non-small |
| | | | | | cell lung carcinoma etc. |
| Firivumab | | mab | human | influenza A virus | — |
| | | | | hemagglutinin | |
| Flanvotumab | | mab | human | TYRP1(glycoprote | melanoma |
| | | | | in 75) | |
| Fletikumab | | mab | human | IL 20 | rheumatoid arthritis |
| Fontolizumab | HuZAF | mab | humanized | IFN-γ | Crohn's disease etc. |
| Foralumab | | mab | human | CD3 epsilon | — |
| Foravirumab | | mab | human | rabies virus | rabies (prophylaxis) |
| | | | | glycoprotein | |
| Fresolimumab | | mab | human | TGF-β | idiopathic pulmonary |
| | | | | | fibrosis, focal |
| | | | | | segmental |
| | | | | | glomerulosclerosis, |
| | | | | | cancer |
| Fulranumab | | mab | human | NGF | pain |
| Futuximab | | mab | chimeric | EGFR | cancer |
| Galcanezumab | | mab | humanized | calcitonin | migraine |
| Galiximab | | mab | chimeric | CD80 | B-cell lymphoma |
| Ganitumab | | mab | human | IGF-1 receptor | cancer |
| | | | | (CD221) | |
| Gantenerumab | | mab | human | beta amyloid | Alzheimer's disease |
| Gavilimomab | | mab | mouse | CD147 (basigin) | graft versus host disease |
| Gemtuzumab | Mylotarg | mab | humanized | CD33 | acute myelogenous |
| ozogamicin | | | | | leukemia |
| Gevokizumab | | mab | humanized | IL-1β | diabetes etc. |
| Girentuximab | Rencarex | mab | chimeric | carbonic anhydrase | clear cell renal cell |
| | | | | 9 (CA-IX) | carcinoma[84] |
| Glembatumumab | | mab | human | GPNMB | melanoma, breast |
| vedotin | | | | | cancer |
| Golimumab | Simponi | mab | human | TNF-α | rheumatoid arthritis, |
| | | | | | psoriatic arthritis, |
| | | | | | ankylosing spondylitis |
| Gomiliximab | | mab | chimeric | CD23 (IgE | allergic asthma |
| | | | | receptor) | |
| Guselkumab | | mab | human | IL23 | psoriasis |
| Ibalizumab | | mab | humanized | CD4 | HIV infection |
| Ibritumomab tiuxetan | Zevalin | mab | mouse | CD20 | non-Hodgkin's |
| | | | | | lymphoma |
| Icrucumab | | mab | human | VEGFR-1 | cancer etc. |
| Idarucizumab | | mab | humanized | dabigatran | reversal of |
| | | | | | anticoagulant effects of |
| | | | | | dabigatran |
| Igovomab | Indimacis-125 | F(ab′)₂ | mouse | CA-125 | ovarian cancer |
| | | | | | (diagnosis) |
| IMAB362 | | mab | human | CLDN18.2 | gastrointestinal |
| | | | | | adenocarcinomas and |
| | | | | | pancreatic tumor |
| Imalumab | | mab | human | MIF | cancer |
| Imciromab | Myoscint | mab | mouse | cardiac myosin | cardiac imaging |
| Imgatuzumab | | mab | humanized | EGFR | cancer |
| Inclacumab | | mab | human | selectin P | cardiovascular disease |
| Indatuximab | | mab | chimeric | SDC1 | cancer |
| ravtansine | | | | | |
| Indusatumab vedotin | | mab | human | GUCY2C | cancer |
| Inebilizumab | | mab | humanized | CD19 | cancer, systemic |
| | | | | | sclerosis, multiple |
| | | | | | sclerosis |
| Infliximab | Remicade | mab | chimeric | TNF-α | rheumatoid arthritis, |
| | | | | | ankylosing spondylitis, |
| | | | | | psoriatic arthritis, |
| | | | | | psoriasis, Crohn's |
| | | | | | disease, ulcerative |
| | | | | | colitis |
| Inolimomab | | mab | mouse | CD25 (α chain of | graft versus host disease |
| | | | | IL-2 receptor) | |
| Inotuzumab | | mab | humanized | CD22 | ALL |
| ozogamicin | | | | | |
| Intetumumab | | mab | human | CD51 | solid tumors (prostate |
| | | | | | cancer, melanoma) |
| Ipilimumab | Yervoy | mab | human | CD152 | melanoma |
| Iratumumab | | mab | human | CD30 (TNFRSF8) | Hodgkin's lymphoma |
| Isatuximab | | mab | chimeric | CD38 | cancer |
| Itolizumab | | mab | humanized | CD6 | — |
| Ixekizumab | | mab | humanized | IL 17A | autoimmune diseases |
| Keliximab | | mab | chimeric | CD4 | chronic asthma |
| Labetuzumab | CEA-Cide | mab | humanized | CEA | colorectal cancer |
| Lampalizumab | | mab | humanized | CFD | geographic atrophy |
| | | | | | secondary to age-related |
| | | | | | macular degeneration |
| Lanadelumab | | mab | human | kallikrein | angioedema |
| Landogrozumab | | mab | humanized | GDF-8 | muscle wasting |
| | | | | | disorders |
| Laprituximab | | mab | chimeric | EGFR | — |
| emtansine | | | | | |
| Lebrikizumab | | mab | humanized | IL-13 | asthma |
| Lemalesomab | | mab | mouse | NCA-90 | diagnostic agent |
| | | | | (granulocyte | |
| | | | | antigen) | |
| Lendalizumab | | mab | humanized | C5 | — |
| Lenzilumab | | mab | human | CSF2 | — |
| Lerdelimumab | | mab | human | TGF beta 2 | reduction of scarring |
| | | | | | after glaucoma surgery |
| Lexatumumab | | mab | human | TRAIL-R2 | cancer |
| Libivirumab | | mab | human | hepatitis B surface | hepatitis B |
| | | | | antigen | |
| Lifastuzumab vedotin | | mab | humanized | phosphate-sodium | cancer |
| | | | | co-transporter | |
| Ligelizumab | | mab | humanized | IGHE | severe asthma and |
| | | | | | chronic spontaneous |
| | | | | | urticaria |
| Lilotomab satetraxetan | | mab | mouse | CD37 | cancer |
| Lintuzumab | | mab | humanized | CD33 | cancer |
| Lirilumab | | mab | human | KIR2D | solid and hematological |
| | | | | | cancers |
| Lodelcizumab | | mab | humanized | PCSK9 | hypercholesterolemia |
| Lokivetmab | | mab | veterinary | Canis lupus | — |
| | | | | familiaris IL31 | |
| Lorvotuzumab | | mab | humanized | CD56 | cancer |
| mertansine | | | | | |
| Lucatumumab | | mab | human | CD40 | multiple myeloma, non- |
| | | | | | Hodgkin's lymphoma, |
| | | | | | Hodgkin's lymphoma |
| Lulizumab pegol | | mab | humanized | CD28 | autoimmune diseases |
| Lumiliximab | | mab | chimeric | CD23 (IgE | chronic lymphocytic |
| | | | | receptor) | leukemia |
| Lumretuzumab | | mab | humanized | ERBB3 (HER3) | cancer |
| MABp1 | Xilonix | mab | human | IL1A | colorectal cancer |
| Mapatumumab | | mab | human | TRAIL-R1 | cancer |
| Margetuximab | | mab | humanized | ch4D5 | cancer |
| Maslimomab | | — | mouse | T-cell receptor | — |
| Matuzumab | | mab | humanized | EGFR | colorectal, lung and |
| | | | | | stomach cancer |
| Mavrilimumab | | mab | human | GMCSF receptor | rheumatoid arthritis |
| | | | | α-chain | |
| Mepolizumab | Bosatria | mab | humanized | IL-5 | asthma and white blood |
| | | | | | cell diseases |
| Metelimumab | | mab | human | TGF beta 1 | systemic scleroderma |
| Milatuzumab | | mab | humanized | CD74 | multiple myeloma and |
| | | | | | other hematological |
| | | | | | malignancies |
| Minretumomab | | mab | mouse | TAG-72 | tumor detection (and |
| | | | | | therapy—) |
| Mirvetuximab | | mab | chimeric | folate receptor | cancer |
| soravtansine | | | | alpha | |
| Mitumomab | | mab | mouse | GD3 ganglioside | small cell lung |
| | | | | | carcinoma |
| Mogamulizumab | | mab | humanized | CCR4 | cancer |
| Monalizumab | | mab | humanized | KLRC1 | — |
| Morolimumab | | mab | human | Rhesus factor | — |
| Motavizumab | Numax | mab | humanized | respiratory | respiratory syncytial |
| | | | | syncytial virus | virus (prevention) |
| Moxetumomab | | mab | mouse | CD22 | cancer |
| pasudotox | | | | | |
| Muromonab-CD3 | Orthoclone | mab | mouse | CD3 | prevention of organ |
| | OKT3 | | | | transplant rejections |
| Nacolomab tafenatox | | Fab | mouse | C242 antigen | colorectal cancer |
| Namilumab | | mab | human | CSF2 | — |
| Naptumomab | | Fab | mouse | 5T4 | non-small cell lung |
| estafenatox | | | | | carcinoma, renal cell |
| | | | | | carcinoma |
| Naratuximab | | mab | chimeric | CD37 | — |
| emtansine | | | | | |
| Narnatumab | | mab | human | RON | cancer |
| Natalizumab | Tysabri | mab | humanized | integrin α4 | multiple sclerosis, |
| | | | | | Crohn's disease |
| Navicixizumab | | mab | chimeric/ | DLL4 | — |
| | | | humanized | | |
| Navivumab | | mab | human | influenza A virus | — |
| | | | | hemagglutinin HA | |
| Nebacumab | | mab | human | endotoxin | sepsis |
| Necitumumab | | mab | human | EGFR | non-small cell lung |
| | | | | | carcinoma |
| Nemolizumab | | mab | humanized | IL31RA | eczema[106] |
| Nerelimomab | | mab | mouse | TNF-α | — |
| Nesvacumab | | mab | human | angiopoietin 2 | cancer |
| Nimotuzumab | Theracim, | mab | humanized | EGFR | squamous cell |
| | Theraloc | | | | carcinoma, head and |
| | | | | | neck cancer, |
| | | | | | nasopharyngeal cancer, |
| | | | | | glioma |
| Nivolumab | Opdivo | mab | human | PD-1 | cancer |
| Nofetumomab | Verluma | Fab | mouse | — | cancer (diagnosis) |
| merpentan | | | | | |
| Obiltoxaximab | | mab | chimeric | Bacillus anthracis | Bacillus anthracis |
| | | | | anthrax | spores |
| Obinutuzumab | Gazyva | mab | humanized | CD20 | Chronic lymphatic |
| | | | | | leukemia |
| Ocaratuzumab | | mab | humanized | CD20 | cancer |
| Ocrelizumab | | mab | humanized | CD20 | rheumatoid arthritis, |
| | | | | | lupus erythematosus |
| | | | | | etc. |
| Odulimomab | | mab | mouse | LFA-1 (CD11a) | prevention of organ |
| | | | | | transplant rejections, |
| | | | | | immunological diseases |
| Ofatumumab | Arzerra | mab | human | CD20 | chronic lymphocytic |
| | | | | | leukemia etc. |
| Olaratumab | | mab | human | PDGF-R α | cancer |
| Olokizumab | | mab | humanized | IL6 | — |
| Omalizumab | Xolair | mab | humanized | IgE Fc region | allergic asthma |
| Onartuzumab | | mab | humanized | human scatter | cancer |
| | | | | factor receptor | |
| | | | | kinase | |
| Ontuxizumab | | mab | chimeric/ | TEM1 | cancer |
| | | | humanized | | |
| Opicinumab | | mab | human | LINGO-1 | multiple sclerosis |
| Oportuzumab monatox | | scFv | humanized | EpCAM | cancer |
| Oregovomab | OvaRex | mab | mouse | CA-125 | ovarian cancer |
| Orticumab | | mab | human | oxLDL | — |
| Otelixizumab | | mab | chimeric/ | CD3 | diabetes mellitus type 1 |
| | | | humanized | | |
| Otlertuzumab | | mab | humanized | CD37 | cancer |
| Oxelumab | | mab | human | OX-40 | asthma |
| Ozanezumab | | mab | humanized | NOGO-A | ALS and multiple |
| | | | | | sclerosis |
| Ozoralizumab | | mab | humanized | TNF-α | inflammation |
| Pagibaximab | | mab | chimeric | lipoteichoic acid | sepsis (Staphylococcus) |
| Palivizumab | Synagis, | mab | humanized | F protein of | respiratory syncytial |
| | Abbosynagis | | | respiratory | virus (prevention) |
| | | | | syncytial virus | |
| Pamrevlumab | | mab | human | CTGF | — |
| Panitumumab | Vectibix | mab | human | EGFR | colorectal cancer |
| Pankomab | | mab | humanized | tumor specific | ovarian cancer |
| | | | | glycosylation of | |
| | | | | MUC1 | |
| Panobacumab | | mab | human | Pseudomonas | Pseudomonas |
| | | | | aeruginosa | aeruginosa infection |
| Parsatuzumab | | mab | human | EGFL7 | cancer |
| Pascolizumab | | mab | humanized | IL-4 | asthma |
| Pasotuxizumab | | mab | chimeric/ | folate hydrolase | cancer |
| | | | humanized | | |
| Pateclizumab | | mab | humanized | LTA | TNF |
| Patritumab | | mab | human | ERBB3 (HER3) | cancer |
| Pembrolizumab | | mab | humanized | PDCD1 | melanoma and other |
| | | | | | cancers |
| Pemtumomab | Theragyn | — | mouse | MUCI | cancer |
| Perakizumab | | mab | humanized | IL 17A | arthritis |
| Pertuzumab | Omnitarg | mab | humanized | HER2/neu | cancer |
| Pexelizumab | | scFv | humanized | C5 | reduction of side effects |
| | | | | | of cardiac surgery |
| Pidilizumab | | mab | humanized | PD-1 | cancer and infectious |
| | | | | | diseases |
| Pinatuzumab vedotin | | mab | humanized | CD22 | cancer |
| Pintumomab | | mab | mouse | adenocarcinoma | adenocarcinoma |
| | | | | antigen | (imaging) |
| Placulumab | | mab | human | human TNF | pain and inflammatory |
| | | | | | diseases |
| Plozalizumab | | mab | humanized | CCR2 | diabetic nephropathy |
| | | | | | and arteriovenous graft |
| | | | | | patency |
| Pogalizumab | | mab | humanized | TNFR superfamily | — |
| | | | | member 4 | |
| Polatuzumab vedotin | | mab | humanized | CD79B | cancer |
| Ponezumab | | mab | humanized | human beta- | Alzheimer's disease |
| | | | | amyloid | |
| Prezalizumab | | mab | humanized | ICOSL | — |
| Priliximab | | mab | chimeric | CD4 | Crohn's disease, |
| | | | | | multiple sclerosis |
| Pritoxaximab | | mab | chimeric | E. coli shiga toxin | — |
| | | | | type-1 | |
| Pritumumab | | mab | human | vimentin | brain cancer |
| PRO 140 | | — | humanized | CCR5 | HIV infection |
| Quilizumab | | mab | humanized | IGHE | asthma |
| Racotumomab | | mab | mouse | N- | cancer |
| | | | | glycolylneuraminic | |
| | | | | acid | |
| Radretumab | | mab | human | fibronectin extra | cancer |
| | | | | domain-B | |
| Rafivirumab | | mab | human | rabies virus | rabies (prophylaxis) |
| | | | | glycoprotein | |
| Ralpancizumab | | mab | humanized | neural apoptosis- | dyslipidemia |
| | | | | regulated | |
| | | | | proteinase 1 | |
| Ramucirumab | Cyramza | mab | human | VEGFR2 | solid tumors |
| Ranibizumab | Lucentis | Fab | humanized | VEGF-A | macular degeneration |
| | | | | | (wet form) |
| Raxibacumab | | mab | human | anthrax toxin, | anthrax (prophylaxis |
| | | | | protective antigen | and treatment) |
| Refanezumab | | mab | humanized | myelin-associated | recovery of motor |
| | | | | glycoprotein | function after stroke |
| Regavirumab | | mab | human | cytomegalovirus | cytomegalovirus |
| | | | | glycoprotein B | infection |
| Reslizumab | | mab | humanized | IL-5 | inflammations of the |
| | | | | | airways, skin and |
| | | | | | gastrointestinal tract |
| Rilotumumab | | mab | human | HGF | solid tumors |
| Rinucumab | | mab | human | platelet-derived | neovascular age-related |
| | | | | growth factor | macular degeneration |
| | | | | receptor beta | |
| Risankizumab | | mab | humanized | IL23A | — |
| Rituximab | MabThera, | mab | chimeric | CD20 | lymphomas, leukemias, |
| | Rituxan | | | | some autoimmune |
| | | | | | disorders |
| Rivabazumab pegol | | mab | humanized | Pseudomonas | — |
| | | | | aeruginosa type III | |
| | | | | secretion system | |
| Robatumumab | | mab | human | IGF-1 receptor | cancer |
| | | | | (CD221) | |
| Roledumab | | mab | human | RHD | — |
| Romosozumab | | mab | humanized | sclerostin | osteoporosis |
| Rontalizumab | | mab | humanized | IFN-α | systemic lupus |
| | | | | | erythematosus |
| Rovalpituzumab | | mab | humanized | DLL3 | — |
| tesirine | | | | | |
| Rovelizumab | LeukArrest | mab | humanized | CD11, CD18 | haemorrhagic shock etc. |
| Ruplizumab | Antova | mab | humanized | CD154 (CD40L) | rheumatic diseases |
| Sacituzumab govitecan | | mab | humanized | tumor-associated | cancer |
| | | | | calcium signal | |
| | | | | transducer 2 | |
| Samalizumab | | mab | humanized | CD200 | cancer |
| Sapelizumab | | mab | humanized | IL6R | — |
| Sarilumab | | mab | human | IL6 | rheumatoid arthritis, |
| | | | | | ankylosing spondylitis |
| Satumomab pendetide | | mab | mouse | TAG-72 | cancer (diagnosis) |
| Secukinumab | | mab | human | IL 17A | uveitis, rheumatoid |
| | | | | | arthritis psoriasis |
| Seribantumab | | mab | human | ERBB3 (HER3) | cancer |
| Setoxaximab | | mab | chimeric | E. coli shiga toxin | — |
| | | | | type-2 | |
| Sevirumab | | — | human | cytomegalovirus | cytomegalovirus |
| | | | | | infection |
| SGN-CD19A | | mab | humanized | CD19 | acute lymphoblastic |
| | | | | | leukemia and B-cell |
| | | | | | non-Hodgkin |
| | | | | | lymphoma |
| SGN-CD33A | | mab | humanized | CD33 | Acute myeloid |
| | | | | | leukemia |
| Sibrotuzumab | | mab | humanized | FAP | cancer |
| Sifalimumab | | mab | humanized | IFN-α | SLE, dermatomyositis, |
| | | | | | polymyositis |
| Siltuximab | | mab | chimeric | IL-6 | cancer |
| Simtuzumab | | mab | humanized | LOXL2 | fibrosis |
| Siplizumab | | mab | humanized | CD2 | psoriasis, graft-versus- |
| | | | | | host disease |
| | | | | | (prevention) |
| Sirukumab | | mab | human | IL-6 | rheumatoid arthritis |
| Sofituzumab vedotin | | mab | humanized | CA-125 | ovarian cancer |
| Solanezumab | | mab | humanized | beta amyloid | Alzheimer's disease |
| Solitomab | | BiTE | mouse | EpCAM | — |
| Sonepcizumab | | — | humanized | sphingosine-1- | choroidal and retinal |
| | | | | phosphate | neovascularization |
| Sontuzumab | | mab | humanized | episialin | — |
| Stamulumab | | mab | human | myostatin | muscular dystrophy |
| Sulesomab | LeukoScan | Fab′ | mouse | NCA-90 | osteomyelitis (imaging) |
| | | | | (granulocyte | |
| | | | | antigen) | |
| Suvizumab | | mab | humanized | HIV-1 | viral infections |
| Tabalumab | | mab | human | BAFF | B-cell cancers |
| Tacatuzumab | AFP-Cide | mab | humanized | alpha-fetoprotein | cancer |
| tetraxetan | | | | | |
| Tadocizumab | | Fab | humanized | integrin αIIbβ3 | percutaneous coronary |
| | | | | | intervention |
| Talizumab | | mab | humanized | IgE | allergic reaction |
| Tamtuvetmab | Tactress | mab | veterinary | CD52 | — |
| Tanezumab | | mab | humanized | NGF | pain |
| Taplitumomab paptox | | mab | mouse | CD19 | cancer[citation needed] |
| Tarextumab | | mab | human | Notch receptor | cancer |
| Tefibazumab | Aurexis | mab | humanized | clumping factor A | Staphylococcus aureus |
| | | | | | infection |
| Telimomab aritox | | Fab | mouse | — | — |
| Tenatumomab | | mab | mouse | tenascin C | cancer |
| Teneliximab | | mab | chimeric | CD40 | autoimmune diseases |
| | | | | | and prevention of organ |
| | | | | | transplant rejection |
| Teplizumab | | mab | humanized | CD3 | diabetes mellitus type 1 |
| Teprotumumab | | mab | human | IGF-1 receptor | hematologic tumors |
| | | | | (CD221) | |
| Tesidolumab | | mab | human | C5 | — |
| Tetulomab | | mab | humanized | CD37 | cancer[141] |
| Tezepelumab | | mab | human | TSLP | asthma, atopic |
| | | | | | dermatitis |
| TGN1412 | | — | humanized | CD28 | chronic lymphocytic |
| | | | | | leukemia, rheumatoid |
| | | | | | arthritis |
| Ticilimumab | | mab | human | CTLA-4 | cancer |
| (=tremelimumab) | | | | | |
| Tigatuzumab | | mab | humanized | TRAIL-R2 | cancer |
| Tildrakizumab | | mab | humanized | IL23 | immunologically |
| | | | | | mediated inflammatory |
| | | | | | disorders |
| Timolumab | | mab | human | AOC3 | — |
| Tisotumab vedotin | | mab | human | coagulation factor | — |
| | | | | III | |
| TNX-650 | | — | humanized | IL-13 | Hodgkin's lymphoma |
| Tocilizumab | Actemra, | mab | humanized | IL-6 receptor | rheumatoid arthritis |
| (=atlizumab) | RoActemra | | | | |
| Toralizumab | | mab | humanized | CD154 (CD40L) | rheumatoid arthritis, |
| | | | | | lupus nephritis etc. |
| Tosatoxumab | | mab | human | Staphylococcus | — |
| | | | | aureus | |
| Tositumomab | Bexxar | — | mouse | CD20 | follicular lymphoma |
| Tovetumab | | mab | human | CD140a | cancer |
| Tralokinumab | | mab | human | IL-13 | asthma etc. |
| Trastuzumab | Herceptin | mab | humanized | HER2/neu | breast cancer |
| Trastuzumab | Kadcyla | mab | humanized | HER2/neu | breast cancer |
| emtansine | | | | | |
| TRBS07 | Ektomab | 3funct | — | GD2 ganglioside | melanoma |
| Tregalizumab | | mab | humanized | CD4 | — |
| Tremelimumab | | mab | human | CTLA-4 | cancer |
| Trevogrumab | | mab | human | growth | muscle atrophy due to |
| | | | | differentiation | orthopedic disuse and |
| | | | | factor 8 | sarcopenia |
| Tucotuzumab | | mab | humanized | EpCAM | cancer |
| celmoleukin | | | | | |
| Tuvirumab | | — | human | hepatitis B virus | chronic hepatitis B |
| Ublituximab | | mab | chimeric | MS4A1 | cancer |
| Ulocuplumab | | mab | human | CXCR4 (CD184) | hematologic |
| | | | | | malignancies |
| Urelumab | | mab | human | 4-1BB (CD137) | cancer etc. |
| Urtoxazumab | | mab | humanized | Escherichia coli | diarrhoea caused by E. |
| | | | | | coli |
| Ustekinumab | Stelara | mab | human | IL-12, IL-23 | multiple sclerosis, |
| | | | | | psoriasis, psoriatic |
| | | | | | arthritis |
| Utomilumab | | mab | human | 4-1BB (CD137) | cancer |
| Vadastuximab talirine | | mab | chimeric | CD33 | — |
| Vandortuzumab | | mab | humanized | STEAP1 | cancer |
| vedotin | | | | | |
| Vantictumab | | mab | human | Frizzled receptor | cancer |
| Vanucizumab | | mab | humanized | angiopoietin 2 | cancer |
| Vapaliximab | | mab | chimeric | AOC3 (VAP-1) | — |
| Varlilumab | | mab | human | CD27 | solid tumors and |
| | | | | | hematologic |
| | | | | | malignancies |
| Vatelizumab | | mab | humanized | ITGA2 (CD49b) | |
| Vedolizumab | Entyvio | mab | humanized | integrin α4β7 | Crohn's disease, |
| | | | | | ulcerative colitis |
| Veltuzumab | | mab | humanized | CD20 | non-Hodgkin's |
| | | | | | lymphoma |
| Vepalimomab | | mab | mouse | AOC3 (VAP-1) | inflammation |
| Vesencumab | | mab | human | NRP1 | solid malignancies |
| Visilizumab | Nuvion | mab | humanized | CD3 | Crohn's disease, |
| | | | | | ulcerative colitis |
| Vobarilizumab | | mab | humanized | IL6R | inflammatory |
| | | | | | autoimmune diseases |
| Volociximab | | mab | chimeric | integrin α5β1 | solid tumors |
| Vorsetuzumab | | mab | humanized | CD70 | cancer |
| mafodotin | | | | | |
| Votumumab | HumaSPECT | mab | human | tumor antigen | colorectal tumors |
| | | | | CTAA16.88 | |
| Xentuzumab | | mab | | IGF1, IGF2 | — |
| Zalutumumab | HuMax-EGFr | mab | human | EGFR | squamous cell |
| | | | | | carcinoma of the head |
| | | | | | and neck |
| Zanolimumab | HuMax-CD4 | mab | human | CD4 | rheumatoid arthritis, |
| | | | | | psoriasis, T-cell |
| | | | | | lymphoma |
| Zatuximab | | mab | chimeric | HER1 | cancer |
| Ziralimumab | | mab | human | CD147 (basigin) | — |
| Zolimomab aritox | | mab | mouse | CD5 | systemic lupus |
| | | | | | erythematosus, graft- |
| | | | | | versus-host disease |

TABLE 5

| Sponsors | Drug code | Trial IDs |
|---|---|---|
| Celltrion | CT-P63 | NCT05017168 |
| Exevir Bio BV | XVR011 | NCT04884295 |
| Jemincare Group | JMB2002 | ChiCTR2100042150 |
| Luye Pharma Group Ltd | LY-CovMab | NA |
| AbbVie | ABBV-47D11 | NCT04644120 |
| HiFiBiO Therapeutics | HFB30132A | NCT04590430 |
| Ology Bioservices | ADM03820 | NCT04592549 |
| Beigene | DXP604 | NCT04669262 |
| Zydus Cadila | ZRC-3308 | NA |
| Hengenix Biotech Inc | HLX70 | NCT04561076 |
| CORAT Therapeutics | COR-101 | NCT04674566 |
| Vir Biotechnol./ | VIR-7832 | NCT04746183 |
| AbCellera/Eli Lilly and | LY-CoV1404, | NCT04634409 |
| Company | LY3853113 | |
| Sorrento Therapeutics, Inc. | COVI-AMG (STI-2020) | NCT04734860 |
| Beigene | DXP593 | NCT04532294; |
| | | NCT04551898 |
| Junshi Biosciences/Eli Lilly | andJS016, LY3832479, LY- | NCT04441918; |
| Company | CoV016 | NCT04441931; |
| | | NCT04427501 |
| Mabwell (Shanghai) Bioscience | MW33 | NCT04533048; |
| Co., Ltd. | | NCT04627584 |
| Toscana Life Sciences Sviluppo | MAD0004J08 | NCT04932850; |
| s.r.l. | | NCT04952805 |
| Bristol-Myers Squibb, | C144-LS and C-135-LS | NCT04700163; |
| Rockefeller University | | Activ-2 study |
| Sinocelltech Ltd. | SCTA01 | NCT04483375; |
| | | NCT04644185 |
| Adagio Therapeutics | ADG20 | NCT04805671 |
| | | NCT04859517 |
| Brii Biosciences | BRII-196 | NCT04479631; |
| | | Activ-3 study |
| Brii Biosciences | BRII-198 | NCT04479644; |
| | | Activ-3 study |
| Tychan Pte. Ltd. | TY027 | NCT04429529; |
| | | NCT04649515 |
| AstraZeneca | AZD7442 (AZD8895 + | NCT04507256; |
| | AZD1061) | NCT04625725; |
| | | NCT04625972 |
| Celltrion | CT-P59 | NCT04525079; |
| | | NCT04593641; |
| | | NCT04602000 |
| Vir Biotechnol./ | VIR-7831/ | NCT04545060; |
| GlaxoSmithKline | GSK4182136 | Activ-3 study |
| AbCellera/Eli Lilly and | LY-CoV555 | NCT04411628 (Phase |
| Company | (LY3819253); | 1); NCT04427501 (Phase 2); |
| | combination of LY- | NCT04497987 |
| | CoV555 with LY- | (Phase 3); |
| | CoV016 (LY3832479) | NCT04501978 (Activ-3 study); |
| | | NCT04518410 (Phase 2/3) |
| Regeneron | REGN-COV2 | NCT04425629 (Phase |
| | (REGN10933 + | 1/2); NCT04426695 (Phase |
| | REGN10987) | 1/2); NCT04452318 (Phase 3) |

In certain embodiments, an agent, such as an anti-inflammatory agent or bioactive lipid, is used to increase the expression level and/or duration of any the therapeutic protein (or biologically active nucleic acid molecules) expressed from the non-viral vectors in the methods herein. In work conducted during the development of embodiments, herein, the anti-inflammatory agents (AILs) and bioactive lipids in Table 6 below were tested, and the ones in black were found to be successful agents.

TABLE 6
AILs and bioactive lipids tested

| | | |
|---|---|---|
| Docosahexaenoic Acid | (DHA) | 3/10/15% |
| Eicosapenaenoic Acid | (EPA) | 10/15% |
| Alpha Linolenic Acid | (ALA) | 3/10/15% |
| Maresin 1 | (MAR1) | 3% |
| Lipoxin A4 | (LA4) | 2% |
| 15-deoxy-12,14-Prostaglandin J2 | (15d) | 3% |
| Arachidonic Acid | (AA) | 10/15% |
| Eicosatetraynoic Acid | (ETA) | 10% |
| Docosapentaenoic Acid | (DPA) | 10/15% |
| Stearidonic Acid | (SA) | 10% |
| Retinoic Acid | (RA) | 10% |
| Trans Retinal | (TA) | 10% |
| 2-Arachidonoyl Glycerol | (AG) | 10% |
| Diallyl Disulfide | (DADS) | 10% |
| 3,3 Diindolylmethane | (DIM) | 10% |
| Prostaglandin E2 | (PE2) | 10% |
| Oleic Acid | (OA) | 5/10/15/30/50% |
| Alpha Tocopherol | (AT) | 2.5% |
| Sphingosine-1-Phosphate | (S-1-P) | 10% |
| Palmitoyl Sphingomyelin | (SPH) | 10% |

Red = Unsuccessful in Culture

### EXAMPLES

In the Examples below, the dexamethasone is water-soluble dexamethasone which contains dexamethasone complexed to cyclodextrin to make it soluble. The dexamethasone palmitate is dexamethasone 21-palmitate.

### Example 1

### Multiple-MAb Expression

This Example describes in vivo expression of multiple unique monoclonal antibodies following serial treatments in mice over a 4 week treatment course.

Experimental Methods: On day 0, three mice per group were given IP injections of dexamethasone (40 mg/kg) two hours prior to sequential IV injection of lipids (1000 nmol DOTAP SUV with 2.5 mol % dexamethasone palmitate and 1000 nmol DMPC (1,2-Dimyristoyl-SN-glycero-3-phosphocholine) neutral lipid with 5 mol % dexamethasone palmitate), followed two hours later by 75 mg of a single plasmid DNA (pDNA) containing 5J8 and anti-IL5 cDNAs (“5J8-IL5”). These mice were again re-treated on days 7, 14, and 21 with IP dexamethasone and IV lipid and sequential pDNA as before, however with pDNA(s) containing the following cDNAs at indicated doses: Day 7: 88 mg B38-lambda anti-CoV2 “B38 Lambda”, Day 14: 44 mg B38-lambda anti-CoV2, and 44 mg of a single pDNA containing two copies of anti-IL5 cDNA (IL5-IL5), Day 21: 44 mg rituximab (aCD20 dual), and 44 mg H4 anti-CoV2 (“H4”). Serum levels of mAb proteins were measured by ELISA 24 hours after each treatment and every 2-3 weeks thereafter. Group mean+/−SEM serum levels of target proteins are shown in the graph. The displayed “Days after injection” time points are all relative to the initial injection of pDNA containing 5J8 and anti-IL5 cDNAs at Day 0.

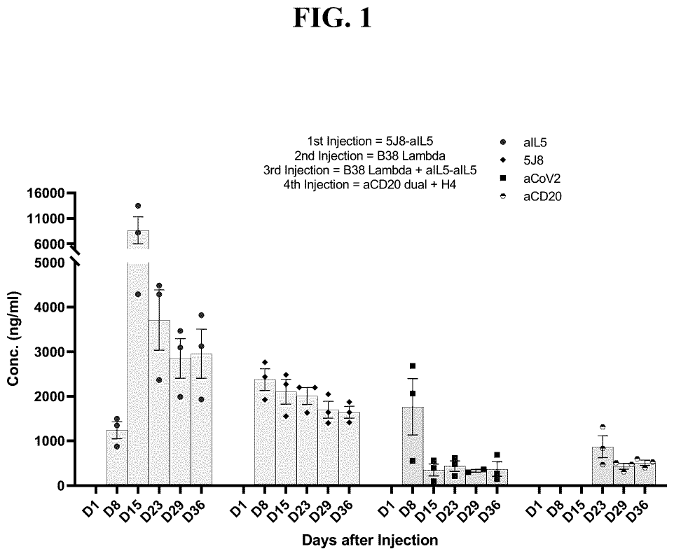

The results are shown in FIG. 1, and demonstrate that serial injection of different DNA mAb vectors on a weekly basis can produce ongoing therapeutic levels of four different intact monoclonal antibodies in individual mice.

### Example 2

### Multiple-MAb Expression

This Example describes in vivo expression of multiple unique monoclonal antibodies following serial treatments in mice over a 6 week treatment course.

Experimental methods: On day 0, three mice per group were given IP injections of dexamethasone (40 mg/kg) two hours prior to sequential IV injection of lipids (1000 nmol DOTAP SUV with 2.5 mol % dexamethasone palmitate and 1000 nmol DMPC (1,2-Dimyristoyl-SN-glycero-3-phosphocholine) neutral lipid with 5 mol % dexamethasone palmitate), followed two hours later by 44 mg each of pDNA containing anti-IL5 and 5J8 cDNAs (“aIL5+5J8”). These same mice were similarly re-treated on days 7, 14, with IP dexamethasone and IV lipid and sequential pDNA as before, however with pDNA(s) containing the following cDNAs at indicated doses: Day 7: 75 mg of the anti-Sars-Cov-2 monoclonal antibody B38 Kappa cDNA (“B38-Kappa”), Day 14: 44 mg of a single pDNA containing two copies of rituximab cDNA (“aCD20-aCD20”), and 44 mg of a single pDNA containing two copies of 5J8 (“5J8-5J8”). Serum levels of mAb proteins were measured by ELISA 24 hours one day following the second treatment (day 8) and every 1-2 weeks thereafter. Group mean+/−SEM serum levels of target proteins are shown in the graph. The indicated time points are all relative to the initial injection of pDNAs-containing anti-IL5 and 5J8 cDNAs at Day 0.

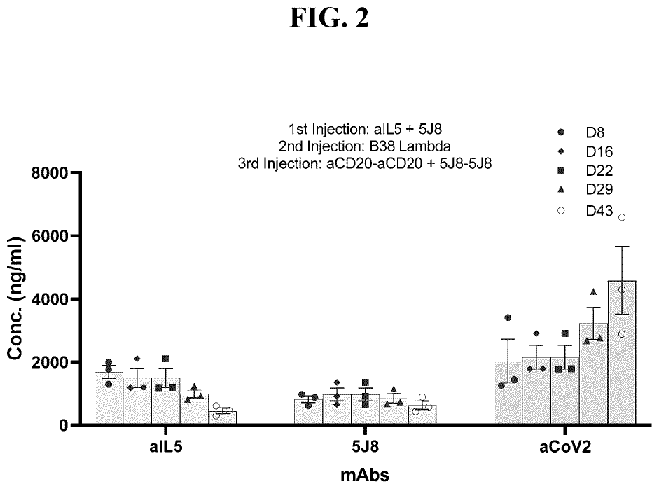

Results are shown in FIG. 2, and demonstrate that serial injection of different DNA mAb vectors injected on a weekly basis can produce ongoing therapeutic levels of four different intact monoclonal antibodies in individual mice.

### Example 3

### Multiple-MAb Expression

This Example describes in vivo expression of multiple unique monoclonal antibodies following serial treatments in mice over a 3 week treatment course.

Experimental Methods:

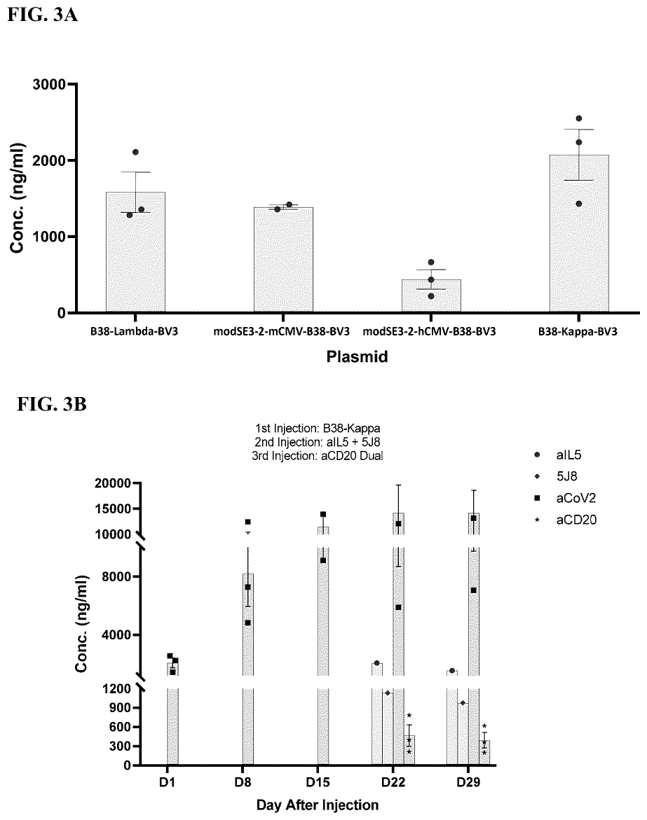

With regard to FIG. 3A: On day 0, 4 groups of three mice per group were given IP injections of dexamethasone (40 mg/kg) two hours prior to sequential IV injection of lipids (1000 nmol DOTAP SUV with 2.5 mol % dexamethasone palmitate and 1000 nmol DMPC (1,2-Dimyristoyl-SN-glycero-3-phosphocholine) neutral lipid with 5 mol % dexamethasone palmitate), followed two hours later by 75 mg of one of four separate pDNA containing anti-Sars-Cov-2 monoclonal antibody B38 cDNA as follows: Group 1: B38-Lambda-BV3, Group 2: modSE3-2-mCMV-B38-BV3, Group 3: modSE3-2-hCMV-B38-BV3, and Group 4: B38-Kappa-BV3. Serum levels of anti-CoV2 mAb proteins were measured by ELISA 24 hours after the initial treatment, and are displayed as group mean+/−SEM.

With regard to FIG. 3B: These same mice were similarly treated on days 7 and 14 with IP dexamethasone and IV lipid and sequential pDNA as before, however with pDNA(s) containing the following cDNAs at indicated doses: Day 7: 44 mg anti-TL5 (“aIL5”) and 44 mg 5J8 (“5J8”). Day 14: 88 mg rituximab (“aCD20 Dual”).

Serum levels of anti-CoV2 mAb proteins were measured by ELISA 24 hours after the initial treatment and weekly thereafter. Serum levels of anti-IL5, 5J8, and rituximab were determined on days 22 and 29, and are displayed as group mean+/−SEM. The indicated time points in FIGS. 3A and 3B are all relative to the initial injection of pDNAs-containing anti-IL5 and 5J8 cDNAs at Day 0.

These results, shown in FIGS. 3A and 3B, demonstrate: A) that various configurations of pDNA expression vectors result in disparate expression levels of target proteins, and B) that serial injection of pDNA mAb vectors encoding for different mAb clones can produce significant ongoing serum levels of four different intact monoclonal antibodies in individual mice.

### Example 4

### Multiple-MAb Expression

This Example describes in vivo expression of multiple unique monoclonal antibodies following serial treatments in mice over a 3 week Treatment Course.

Experimental methods: On day 0, three groups of mice each containing three mice per group, were similarly given IP injections of dexamethasone (40 mg/kg) two hours prior to sequential IV injection of lipids (1000 nmol DOTAP SUV with 2.5 mol % dexamethasone palmitate and 1000 nmol DMPC (1,2-Dimyristoyl-SN-glycero-3-phosphocholine) neutral lipid) with 5 mol % dexamethasone palmitate, followed by the following pDNA(s) containing the following cDNAs at indicated doses: 44 mg of a single pDNA containing two copies of 5J8 cDNA (“5J8-5J8”), and 44 mg of a single pDNA containing two copies of anti-IL5 cDNA (“aIL5-aIL5”). These same groups of mice were treated on days 7, 14, with IP dexamethasone and IV lipid and sequential pDNA as before, however with pDNA(s) containing the following cDNAs at indicated doses: Group 1: Day 7-44 mg of rituximab cDNA (“aCD20-dual”) and 44 mg of the B38 anti-SARS CoV2 cDNAs (“B38-Tag”), Day 14-88 mg of the anti-Sars-Cov-2 monoclonal antibody (“H4”). Group 2: Day 7-44 mg of a single pDNA containing two copies of rituximab cDNAs (“aCD20-aCD20”) and 44 mg of the anti-Sars-Cov-2 monoclonal antibody B38 Kappa cDNA (“B38-Kappa”) cDNAs (“B38-Tag”), Day 14-88 mg of the anti-Sars-Cov-2 monoclonal antibody H4 cDNA (“H4”). Group 3: Day 7-44 mg of rituximab cDNA (“aCD20-dual”) and 44 mg of the B38 anti-SARS CoV2 cDNAs (“B38-Tag”), Day 14—No Treatment.

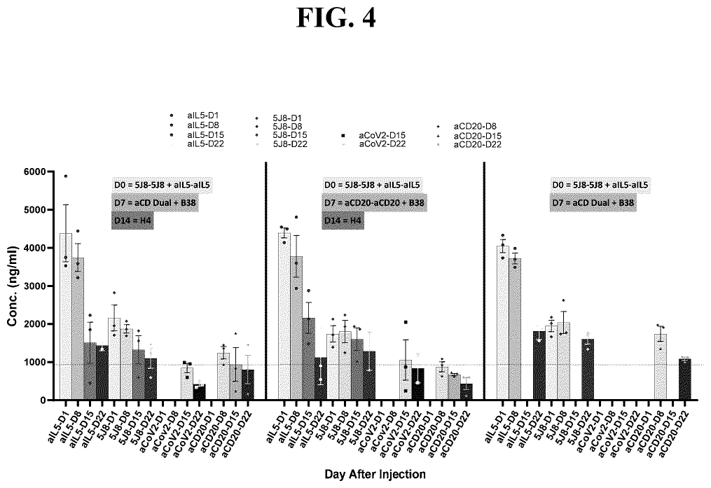

Serum levels of mAb proteins were measured by ELISA on days 1, 8, and 15. The indicated time points are all relative to the initial injection of pDNAs containing 5J8 and aIL5 cDNAs. Results are shown in FIG. 4, which show that serial injection of different DNA mAb vectors on a weekly basis can produce significant ongoing serum levels of four different intact monoclonal antibodies in individual mice.

### Example 5

### Multiple Protein Expression

This Example describes in vivo expression of multiple unique monoclonal antibodies following serial treatments in Mice over a 3 week Treatment.

Experimental methods:

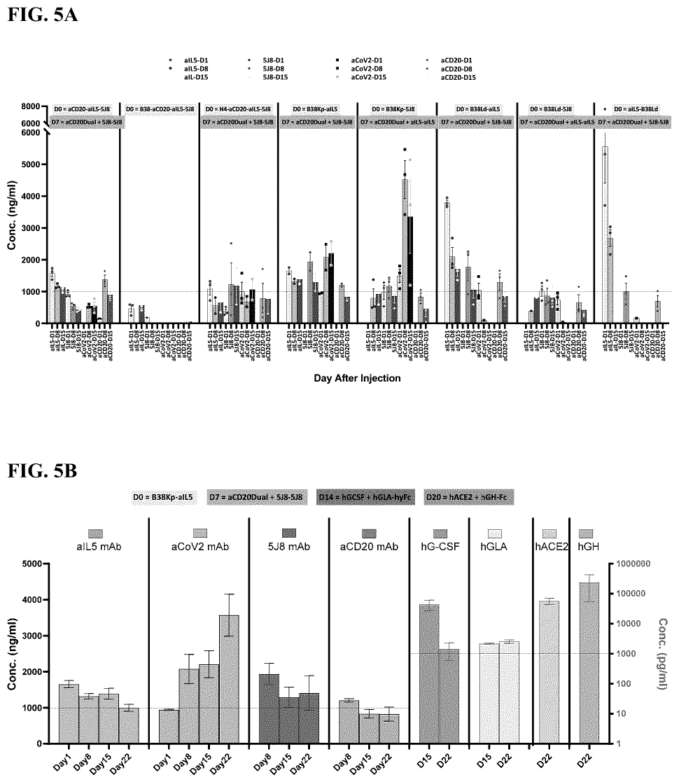

With regard to FIG. 5A: On day 0, eight groups of mice, each containing three mice per group, were given IP injections of dexamethasone (40 mg/kg) two hours prior to sequential IV injection of lipids (1000 nmol DOTAP SUV with 2.5 mol % dexamethasone palmitate and 1000 nmol DMPC (1,2-Dimyristoyl-SN-glycero-3-phosphocholine neutral lipid) with 5 mol % dexamethasone palmitate, followed by the following pDNA(s) containing the following cDNAs at indicated doses: Group 1: 88 mg of a single pDNA encoding rituximab, anti-IL5 and 5J8 cDNAs (“maCD20-haIL5-m5J8”); Group 2: 88 mg of a single pDNA encoding the anti-SARS-Cov-2 monoclonal antibody B38 Lambda cDNA (“B38-Kappa”), rituximab, anti-IL5 and 5J8 cDNAs (“mB38Ld-maCD20-haIL5-m5J8”); Group 3: 88 mg of a single pDNA encoding the anti-Sars-Cov-2 monoclonal antibody H4 cDNA (“mH4”), rituximab, anti-IL5 and 5J8 cDNAs (“mH4-maCD20-haIL5-m5J8”); Group 4: 88 mg of a single pDNA encoding the anti-Sars-Cov-2 monoclonal antibody B38 Kappa cDNA (“B38-Kappa”) and anti-IL5 cDNAs (“mB38Kp-haIL5”); Group 5: 88 mg of a single pDNA encoding the anti-Sars-Cov-2 monoclonal antibody B38 Kappa cDNA (“B38-Kappa”) and 5J8 cDNAs (“mB38Kp-m5J8”); Group 6: 88 mg of a single pDNA encoding the anti-Sars-Cov-2 monoclonal antibody B38 Lambda cDNA (“B38-Lambda”) and anti-IL5 cDNAs (“mB38Ld-maIL5”); Group 7: 88 mg of a single pDNA encoding the anti-Sars-Cov-2 monoclonal antibody B38 Lambda cDNA and 5J8 cDNAs (“mB38Ld-m5J8”); Group 8: 88 mg of a single pDNA encoding anti-IL5 and B38 Lambda cDNAs (“maIL5-mB38Ld”). Some of these same groups of mice were re-treated on days 7 and/or day 14, with IP dexamethasone and IV lipid and sequential pDNA as before, however with pDNA(s) containing the following cDNAs at indicated doses:

Group 1: Day 7-44 mg rituximab (“aCD20-dual”) and 44 mg of a single pDNA containing anti-SARS-CoV2 mAb H4, Day 14—No Treatment. Group 2: Day 7—No Treatment, Day 14—No Treatment. Groups 3, 4: Day 7-44 mg rituximab (“aCD20-dual”) and 44 mg of a single pDNA containing two copies of 5J8 cDNAs (“5J8-5J8”), Day 14-44 mg human G-CSF (“GCSF”) and 44 mg human alpha-glactosidase A (“GLA”) (“hGLA-hyFc”), Day 21-44 mg human Ace2 (“hACE2”) and 44 mg human growth hormone (“hGH”) (“hGH-Fc”). Groups 5: Day 7-44 mg rituximab (“aCD20-dual”) and 44 mg of a single pDNA containing two copies of anti-IL5 cDNAs (“aIL5-aIL5”), Day 14-44 mg GCSF (“GCSF”) and 44 mg GLA (“GLA”). Groups 6 and 8: Day 7-44 mg rituximab (“aCD20-dual”) and 44 mg of a single pDNA containing two copies of 5J8 cDNAs (“5J8-5J8”), Day 14—No Treatment. Group 7: Day 7-44 mg rituximab (“aCD20-dual”) and 44 mg of a single pDNA containing two copies of anti-IL5 cDNAs (“aIL5-aIL5”), Day 14—No Treatment. Serum levels of anti-CoV2 mAb proteins were measured by ELISA 24 hours after the initial treatment and weekly thereafter. The indicated time points are all relative to the initial injection of pDNAs. Group mean+/−SEM expression levels are indicated on the graph.

With regard to FIG. 5B: Serum from treated mice in treatment group 4 (above) were measured by ELISA for expression of non-monoclonal antibody therapeutic human proteins G-CSF, GLA, GH, and ACE2 in serum at day 15 and day 22 following treatment with GCSF+GLA and ACE2+GH containing pDNAs as indicated.

These results, shown in FIGS. 5A and 5B, demonstrate that serial injection of different DNA mAb vectors on a weekly basis can produce significant ongoing serum levels of a total of four different intact monoclonal antibodies and four other non-monoclonal antibody therapeutic proteins (total of eight therapeutic proteins) in individual mice.

### Example 6

### Multiple-MAb Expression

This Example describes the production of three different monoclonal antibody proteins following a single treatment in Mice.

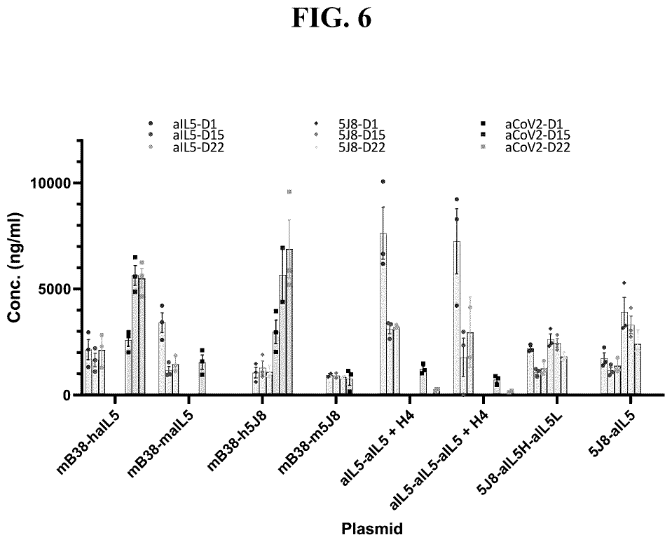

Experimental methods: On day 0, eight groups of mice, each containing three mice per group, were given IP injections of dexamethasone (40 mg/kg) two hours prior to sequential IV injection of lipids (1000 nmol DOTAP SUV with 2.5 mol % dexamethasone palmitate and 1000 nmol DMPC (1,2-Dimyristoyl-SN-glycero-3-phosphocholine neutral lipid with 5 mol % dexamethasone palmitate), followed by the following pDNA(s) containing the following cDNAs at indicated doses: Group 1: 88 mg of a single pDNA encoding anti-SARS-CoV2 B38 kappa and anti-IL5 (“mB38-haIL5”); Group 2: 88 mg of a single pDNA encoding anti-SARS-CoV2 B38 kappa and anti-IL5 (“mB38-maIL5”); Group 3: 88 mg of a single pDNA encoding anti-SARS-CoV2 B38 lambda and anti-influenza A 5J8 (“mB38-h5J8”); Group 4: 88 mg of a single pDNA encoding anti-SARS-CoV2 B38 lambda and anti-influenza A 5J8 (“mB38-m5J8”); Group 5: 44 mg of a single pDNA encoding two copies of anti-IL5 (“aIL5-aIL5”) and 44 mg of a single pDNA encoding anti-SARS-CoV2 (“H4”); Group 6: 44 mg of a single pDNA encoding three copies of anti-IL5 (“aIL5-aIL5-aIL5”) and 44 mg of a single pDNA encoding anti-SARS-CoV2 (“H4”); Group 7: 88 mg of a single pDNA encoding anti-influenza A 5J8 and anti-IL5 (“5J8-aILH-aILL”); Group 8: 88 mg of a single pDNA encoding anti-influenza A 5J8 and anti-IL5 (“5J8-aIL5”). Serum levels of expressed mAb proteins were measured by ELISA 1, 14 and 22 days after the initial treatment. Group mean+/−SEM expression levels are indicated in FIG. 6.

These results, shown in FIG. 6, demonstrate that one dose (e.g., using cationic and neutral lipids) of DNA-encoded mAb vectors, in the form of a single pDNA or composed of multiple pDNAs, can produce sustained expression of a two separate mAbs in mice, and that the structure and composition of the pDNA or pDNAs contribute to mAb expression levels.

### Example 7

### Anti-Sars-CoV2 Protein Expression

This Example describes production of multiple different anti-SARS CoV2 therapeutic proteins separately and in combination following a single treatment in mice.

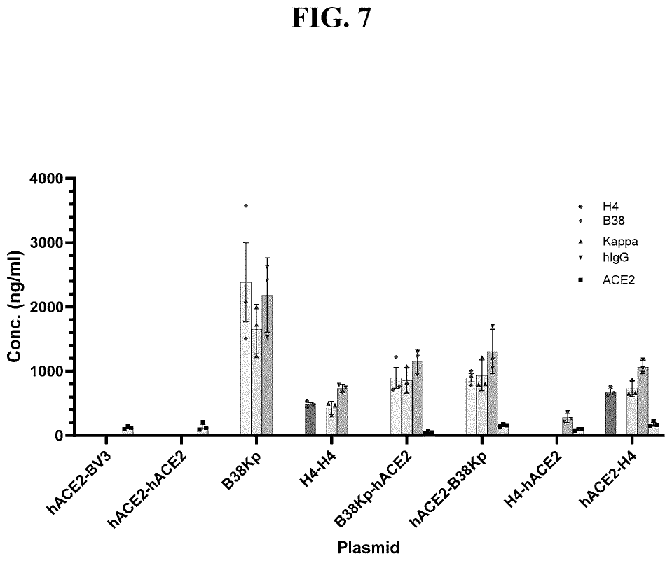

Experimental methods: On day 0, eight groups of mice, each containing three mice per group, were given IP injections of dexamethasone (40 mg/kg) two hours prior to sequential IV injection of lipids (1000 nmol DOTAP SUV with 2.5 mol % dexamethasone palmitate and 1000 nmol DMPC (1,2-Dimyristoyl-SN-glycero-3-phosphocholine neutral lipid with 5 mol % dexamethasone palmitate), followed by injection of 88 mg of a single pDNA encoding the following cDNAs: Group 1: soluble human ACE2 (“hACE2-BV3”), Group 2: two copies of soluble human ACE2 (“hACE2-hACE2”), Group 3: anti-SARS-CoV2 mAb B38 Kappa (“B38Kp”), Group 4: two copies of anti-SARS-CoV2 mAb H4 (“H4-H4”), Group 5: anti-SARS-CoV2 mAb B38 Kappa and soluble human ACE2 (“B38Kp-hACE2”), Group 6: soluble human ACE2 and anti-SARS-CoV2 mAb B38 Kappa (“hACE2-B38Kp”), Group 7: anti-SARS-CoV2 mAb H4 and soluble human ACE2 (“H4-hACE2”), Group 8: soluble human ACE2 and anti-SARS-CoV2 mAb H4 (“hACE2-H4”). Serum expression levels of anti-SARS-CoV2 mAbs were measured by an anti-RBD ELISA using recombinant purified H4 or B38 kappa as standards, or by a non-antigen-specific human IgG or human kappa light chain ELISA. Serum expression levels of soluble human ACE2 were determined by commercial ELISA. Group mean+/−SEM expression levels are indicated in FIG. 7.

The results, in FIG. 7, demonstrate anti-SARS-CoV2 therapeutics (either soluble human ACE2 protein and/or anti-SARS-CoV-2 mAbs reactive to SARS-CoV2 spike protein alone, or in combination) can be produced in animals following a single treatment with a single pDNA vector.

### Example 8

### Anti-Sars-CoV2 Protein Expression

This Example describes production of Multiple anti-SARS CoV2 therapeutics separately and in combination following liposome and dexamethasone treatment in mice.

Experimental methods: On day 0, four groups of mice, each containing three mice per group, were given IP injections of dexamethasone (40 mg/kg) two hours prior to sequential IV injection of lipids (1000 nmol DOTAP SUV with 2.5 mol % dexamethasone palmitate and 1000 nmol DMPC (1,2-Dimyristoyl-SN-glycero-3-phosphocholine neutral lipid with 5 mol % dexamethasone palmitate), followed by injection of 88 mg of a single pDNA encoding the following cDNAs: Group 1: soluble human ACE2-Fc fusion (“shACE2-Fc”), Group 2: soluble human ACE2-Fc fusion LALA variant (“shACE2-Fc-LALA”) Group 3: anti-SARS-CoV2 mAb 4A8 and soluble human ACE2-Fc fusion (“4A8-shACE2-Fc”), Group 4: two copies of soluble human ACE2-Fc fusion (“shACE2-shACE2”).

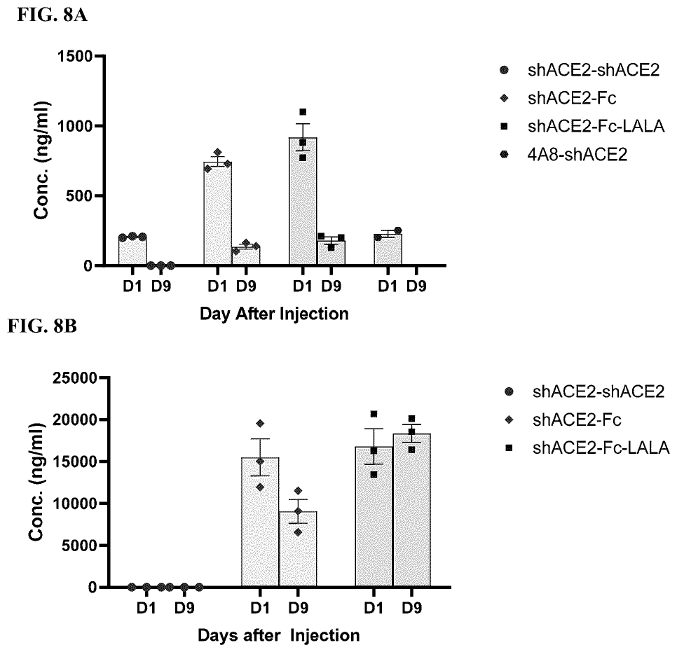

In FIG. 8A, serum expression levels of soluble human ACE2-containing proteins were determined by a SARS-CoV2 RBD-based ELISA on days 1 and 9 following treatment. Group mean+/−SEM expression levels are indicated on the graph.

In FIG. 8B, serum expression levels of soluble human ACE2-Fc fusions were determined in groups 1 thru 3 by an Fc-specific ELISA on days 1 and 9 following treatment. Group mean+/−SEM expression levels are indicated on the graph.

The results, shown in FIGS. 8A and 8B, demonstrate anti-SARS-CoV2 therapeutics (either soluble human ACE2 fusion protein alone, or in combination with the 4A8 mAb reactive against SARS-CoV2 spike protein, may be expressed in vivo following liposome and dexamethasone treatment with a pDNA vector.

### Example 9

### ACE2 Protein Expression

This Example describes production of Human ACE2 and modified variants in mice.

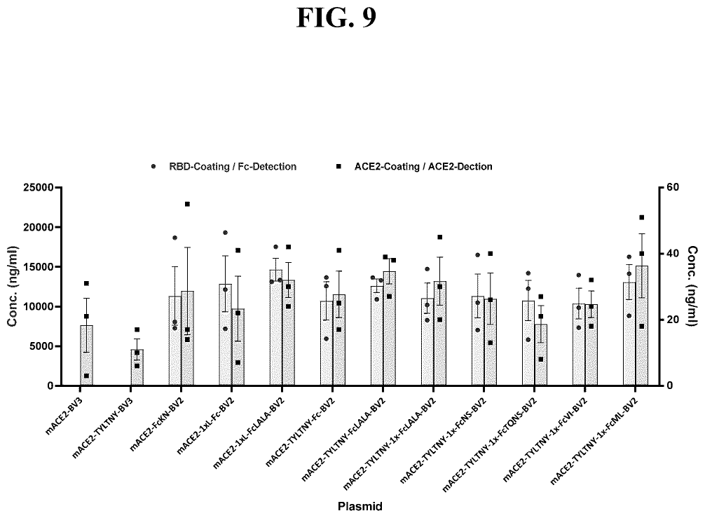

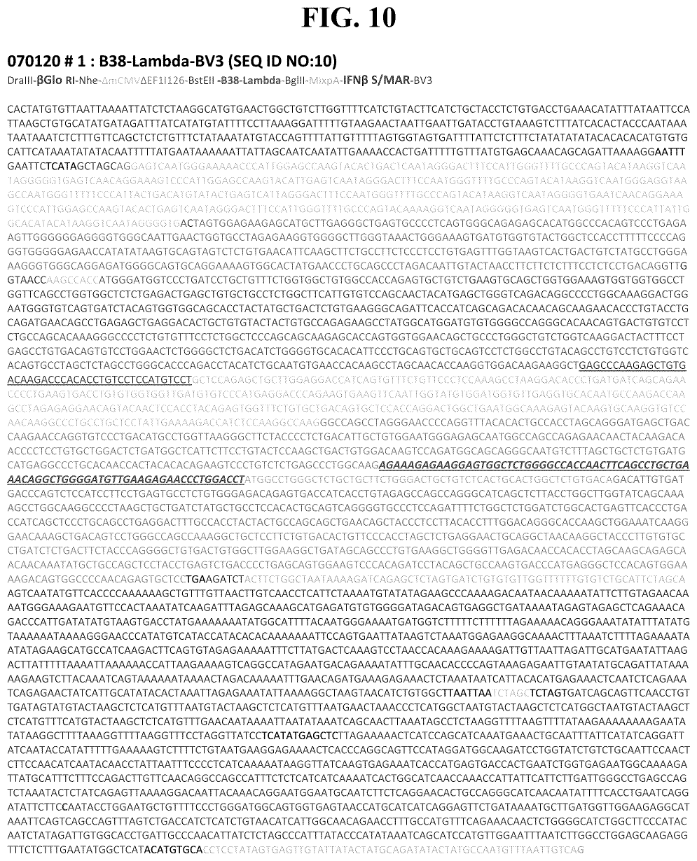

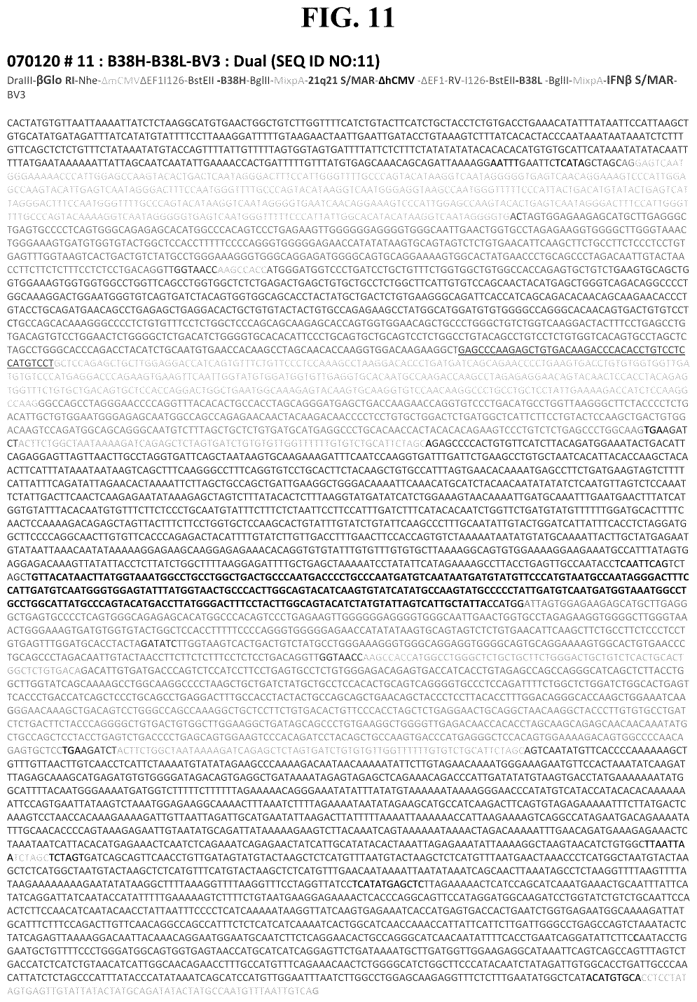

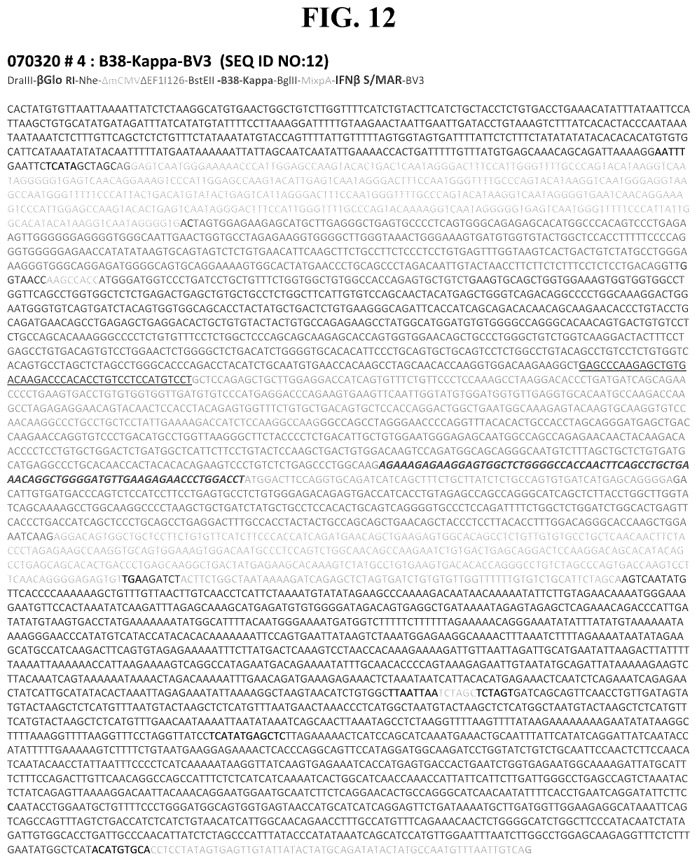

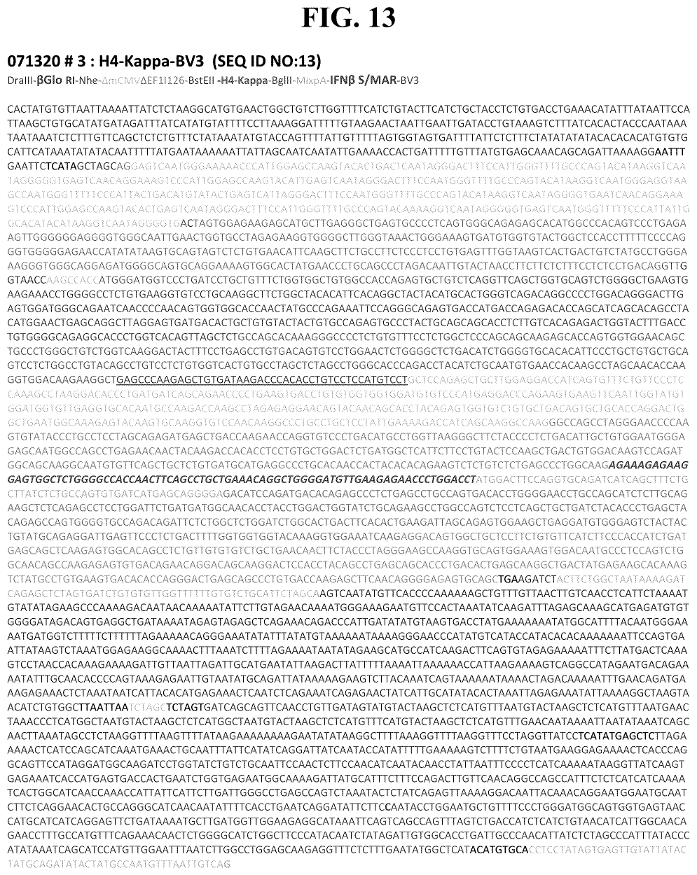

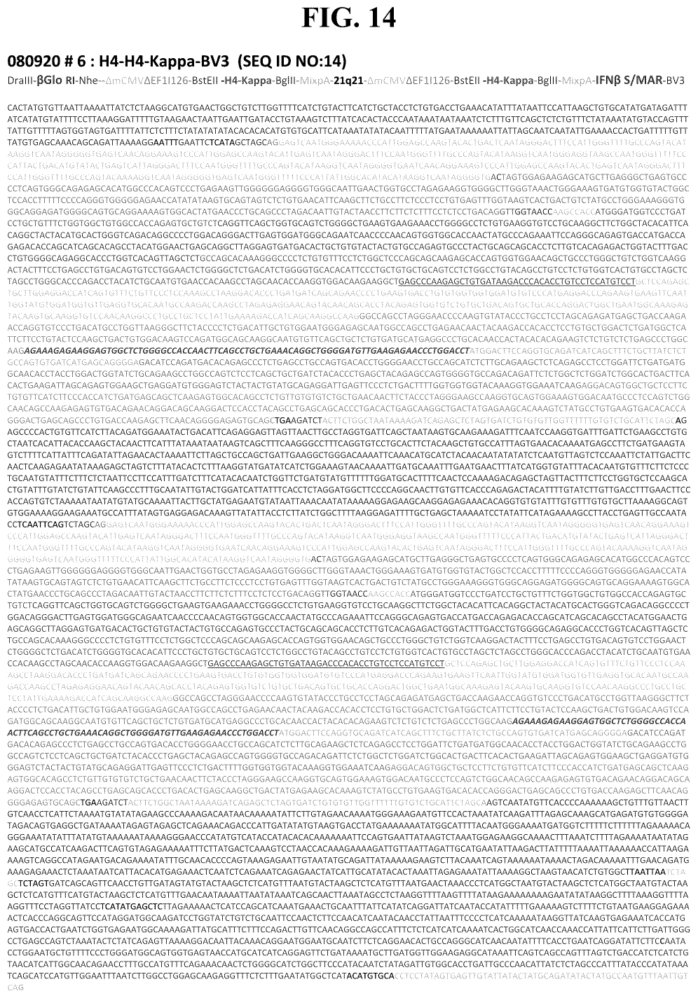

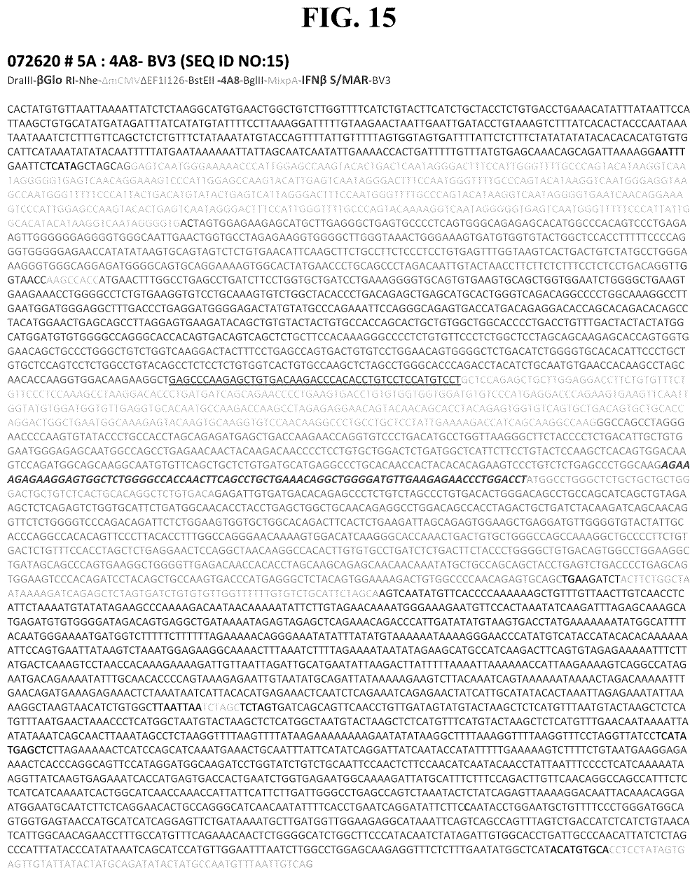

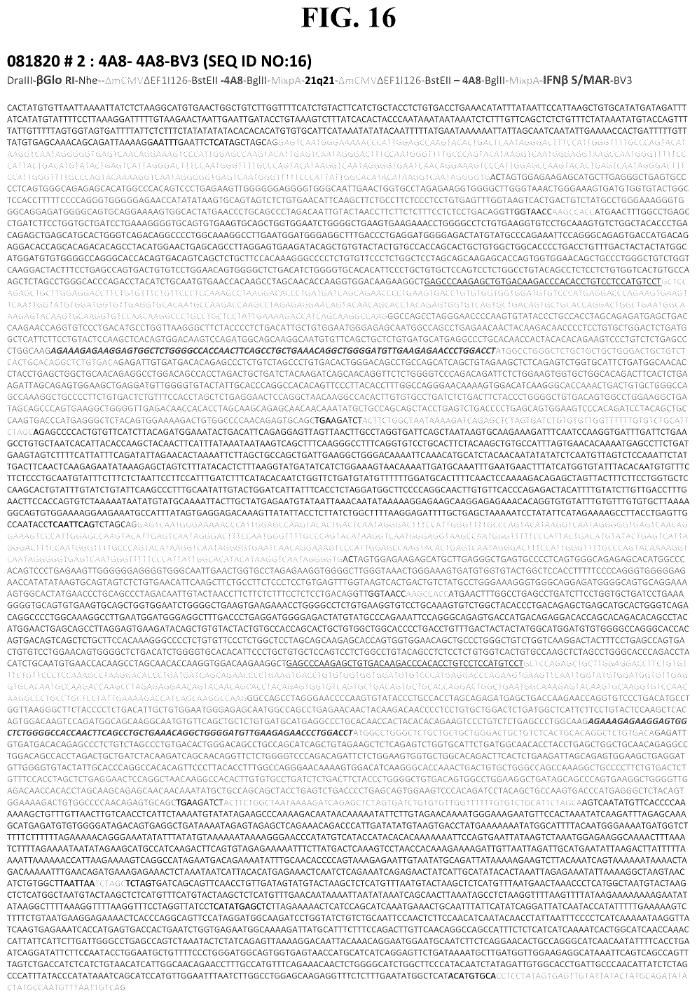

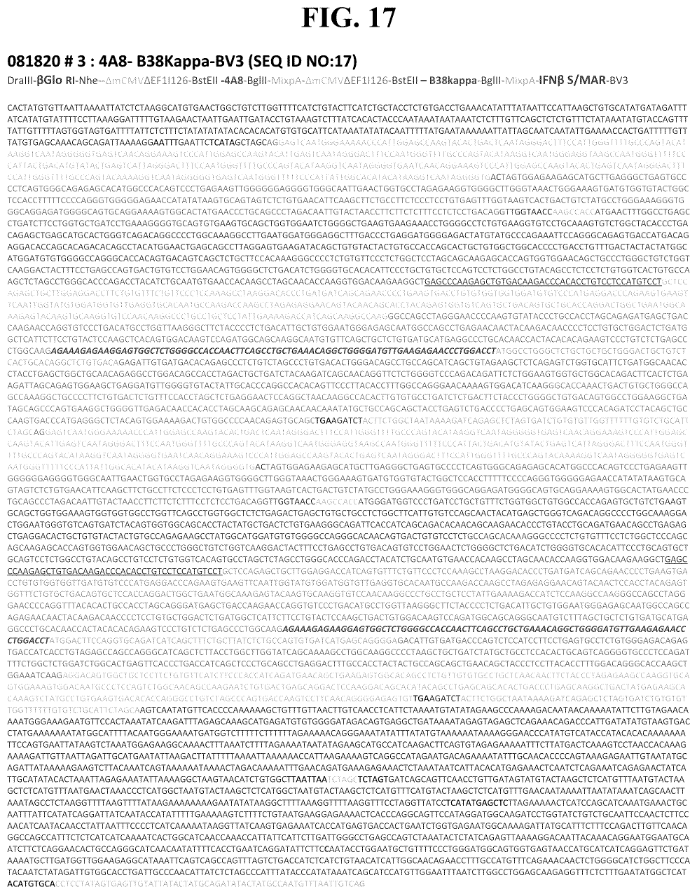

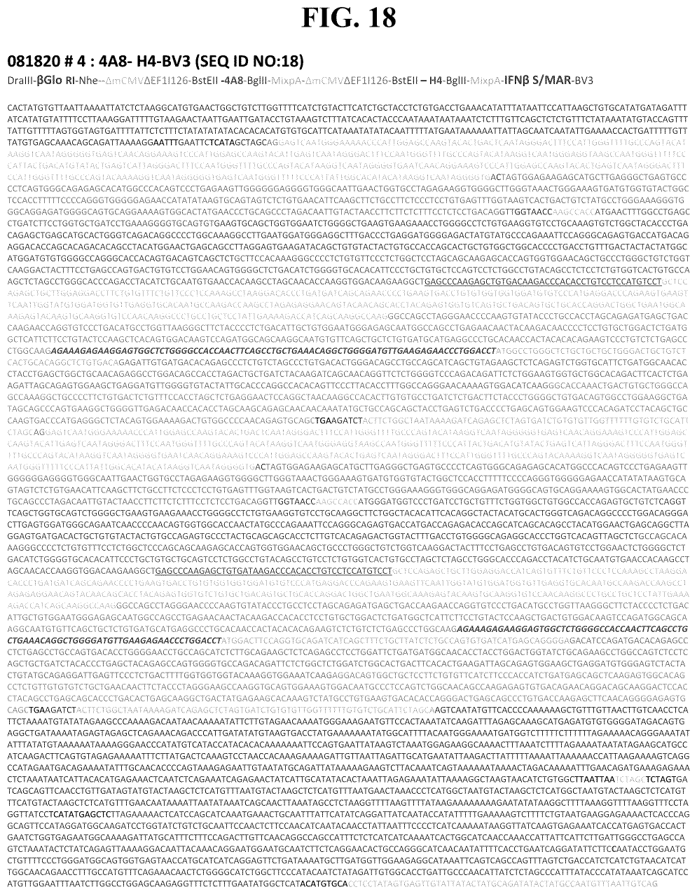

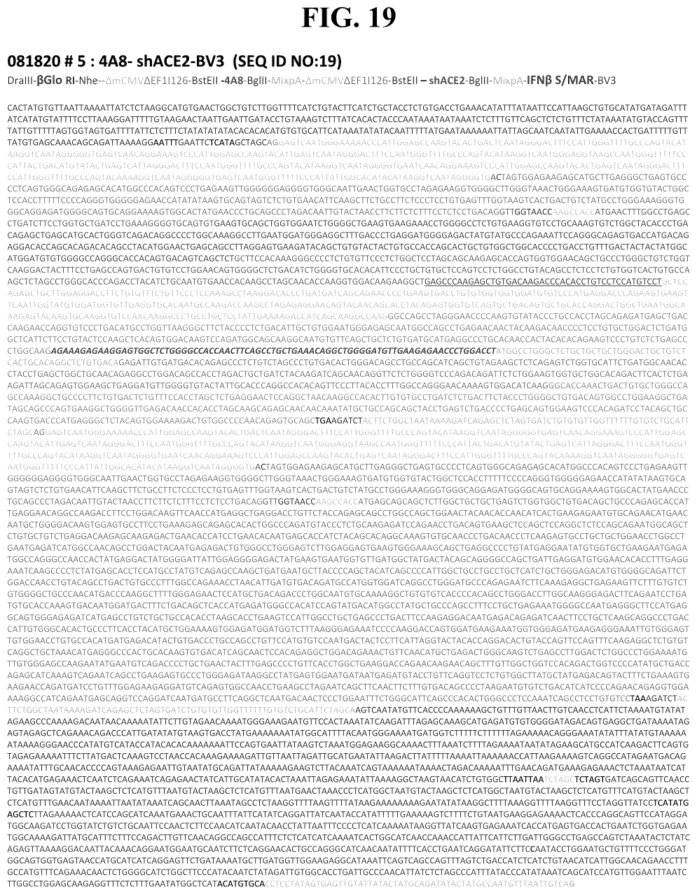

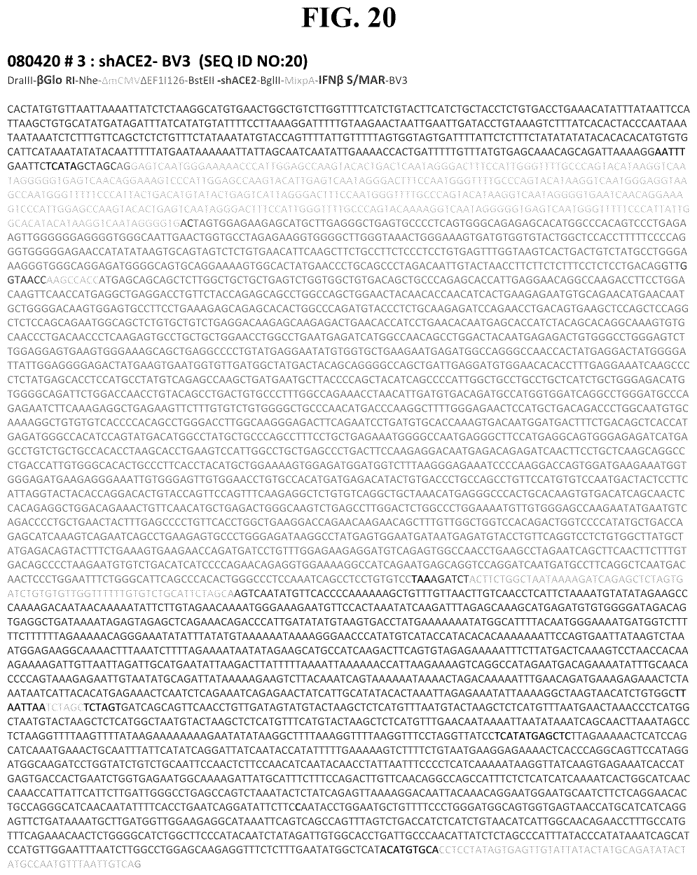

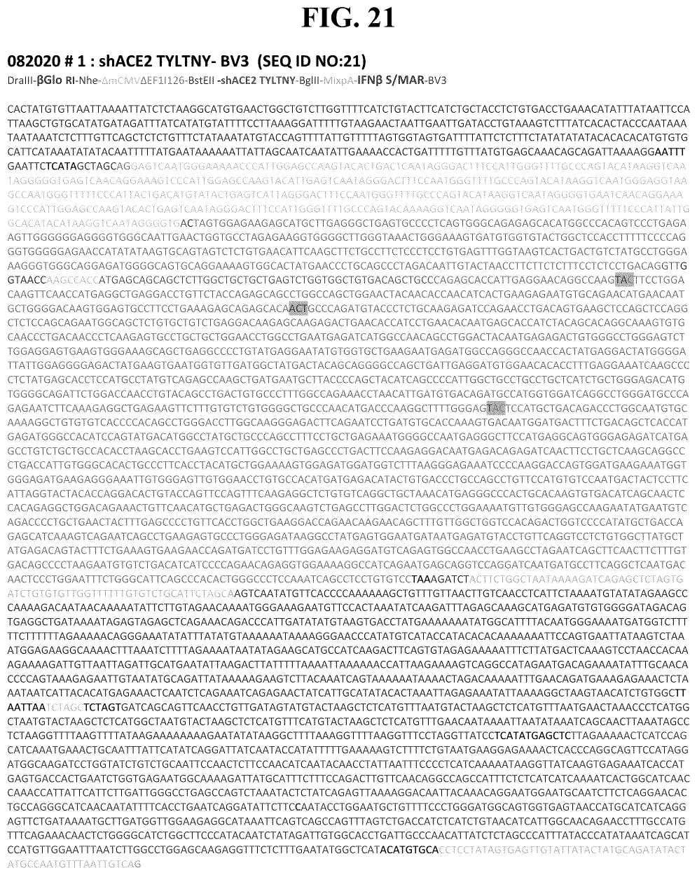

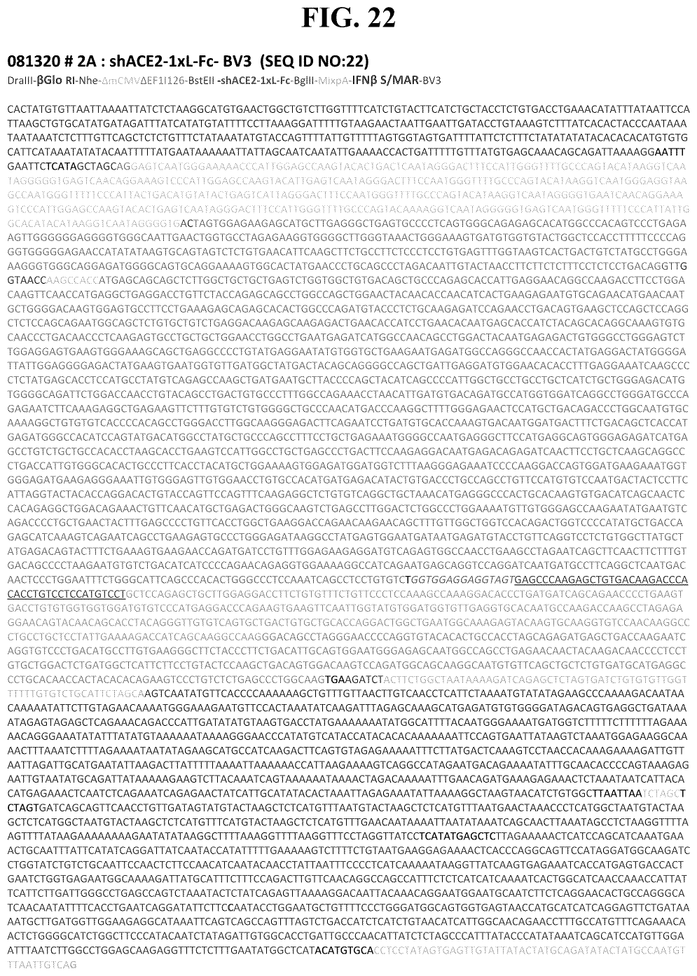

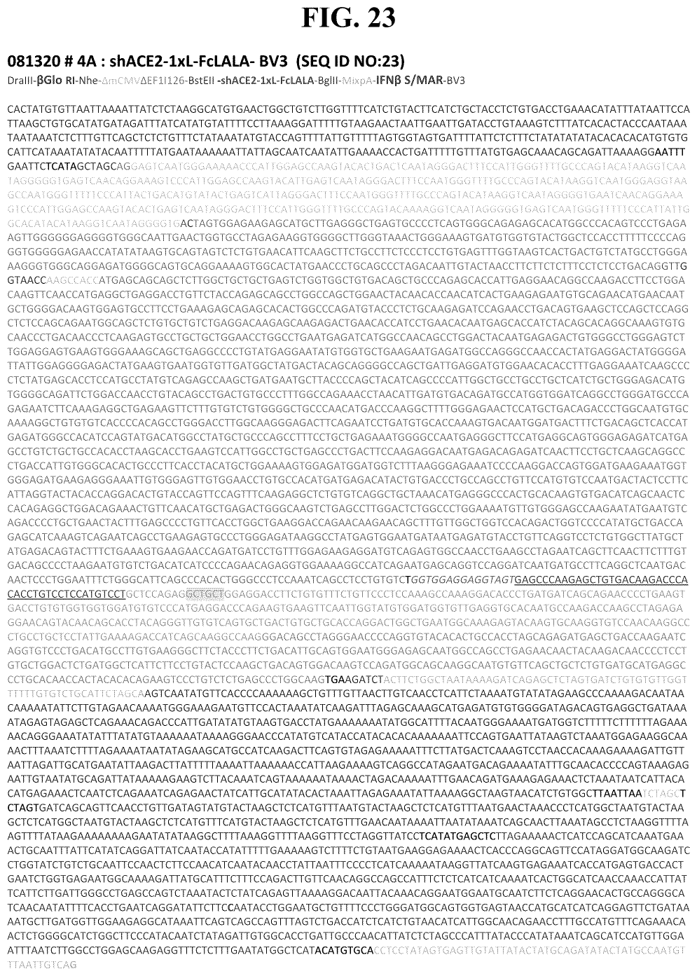

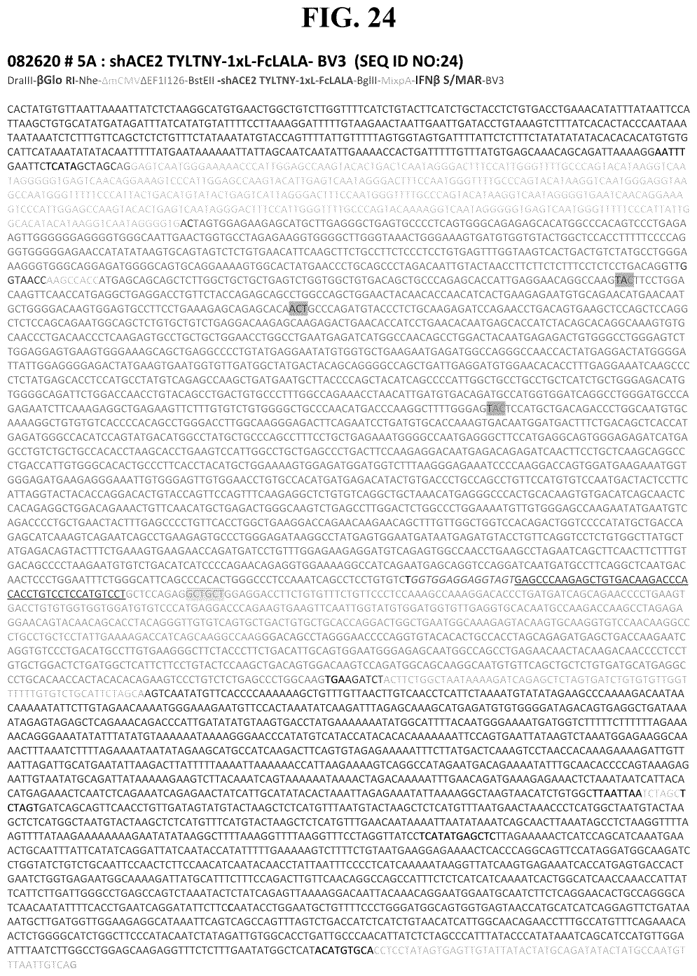

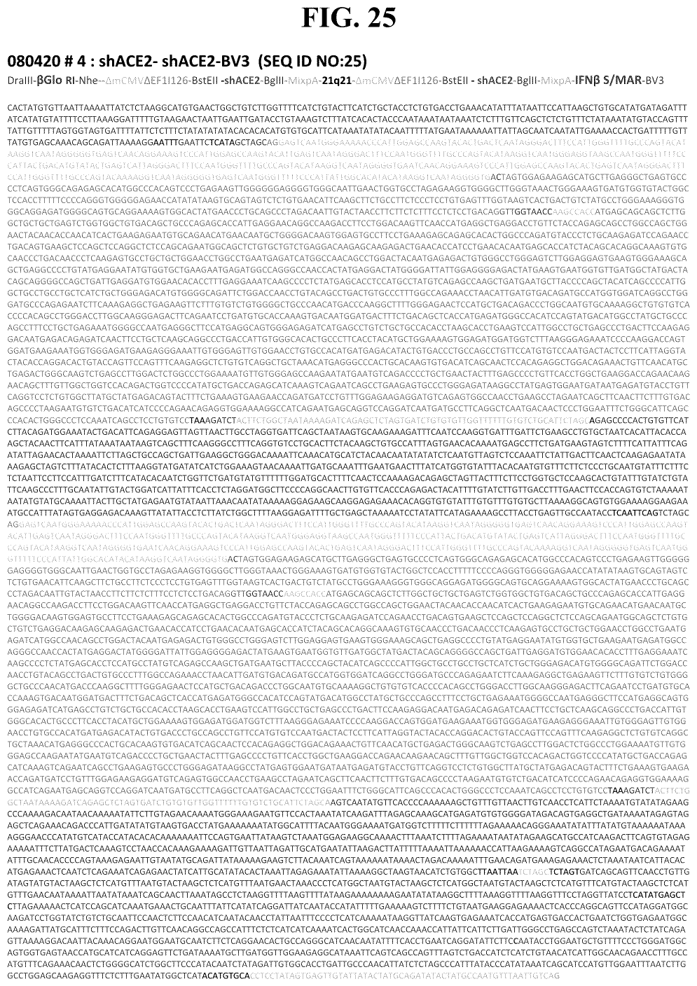

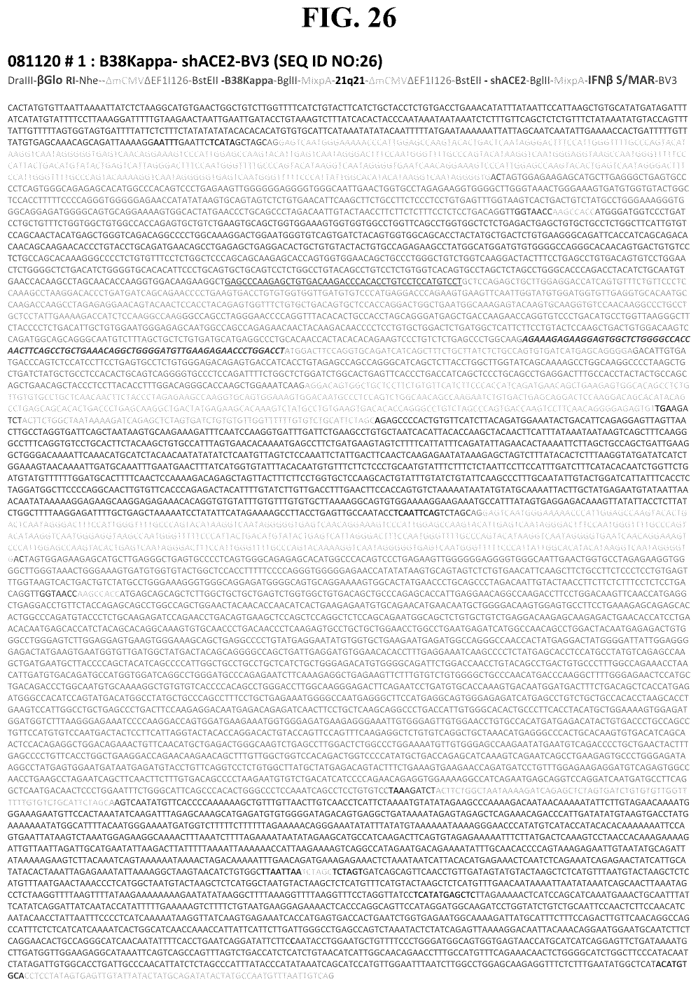

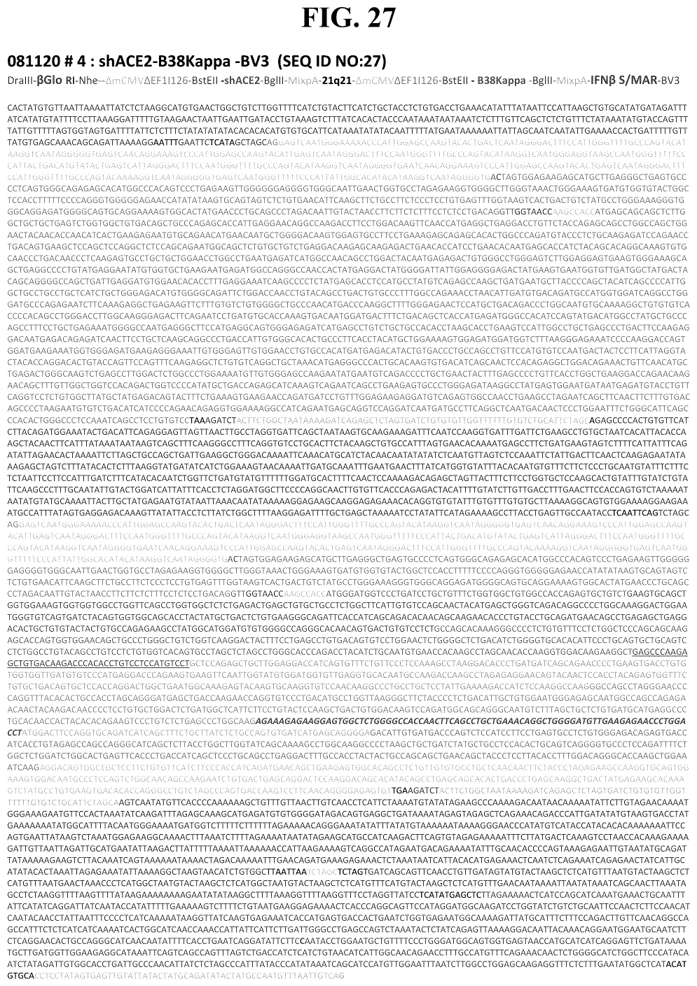

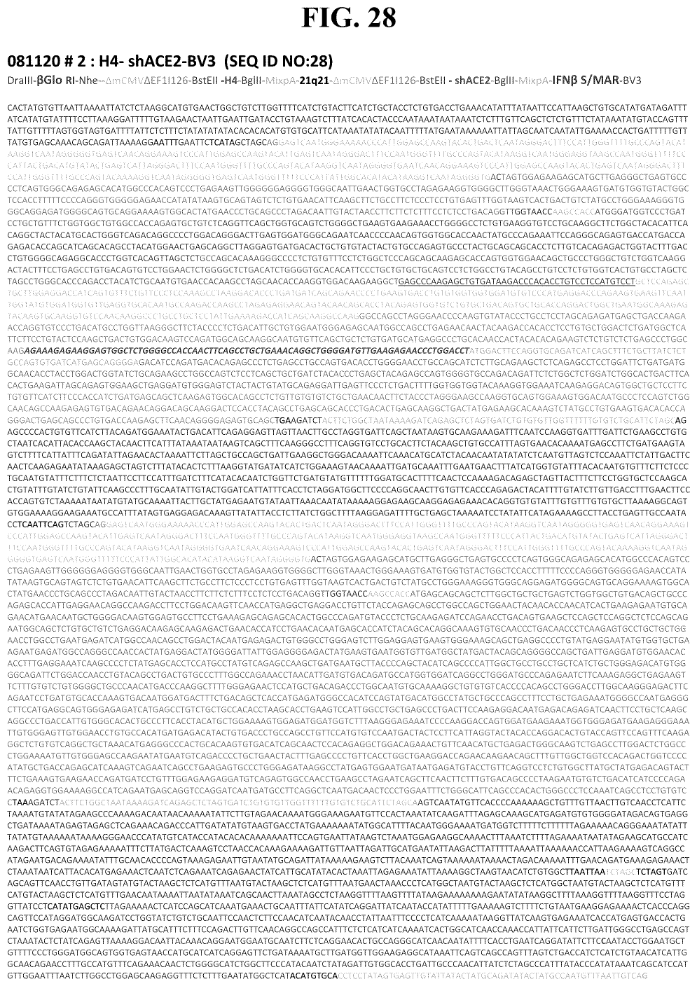

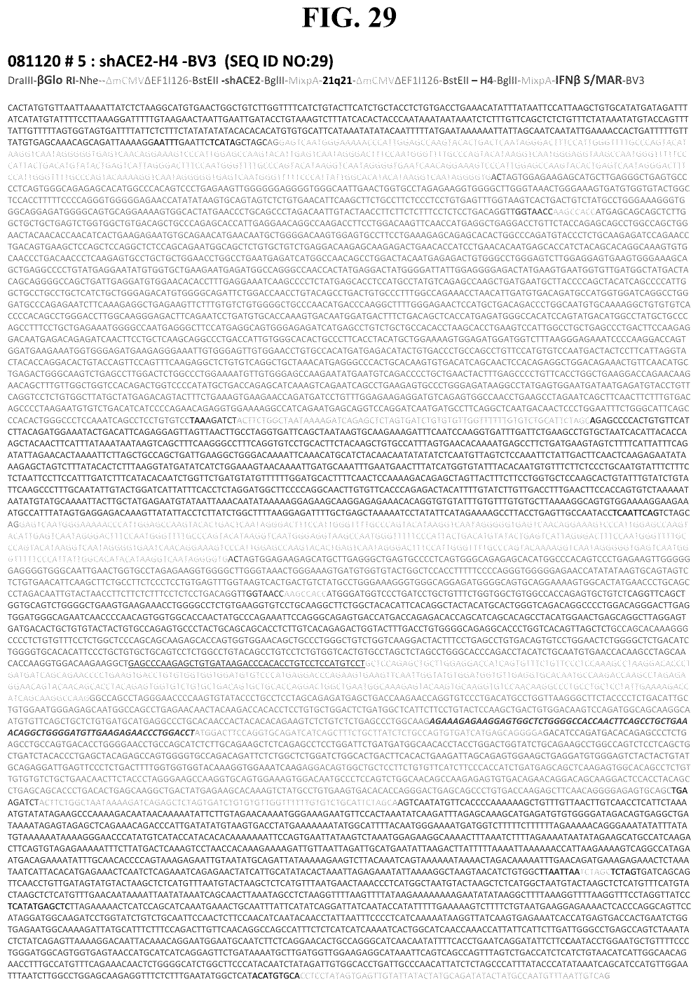

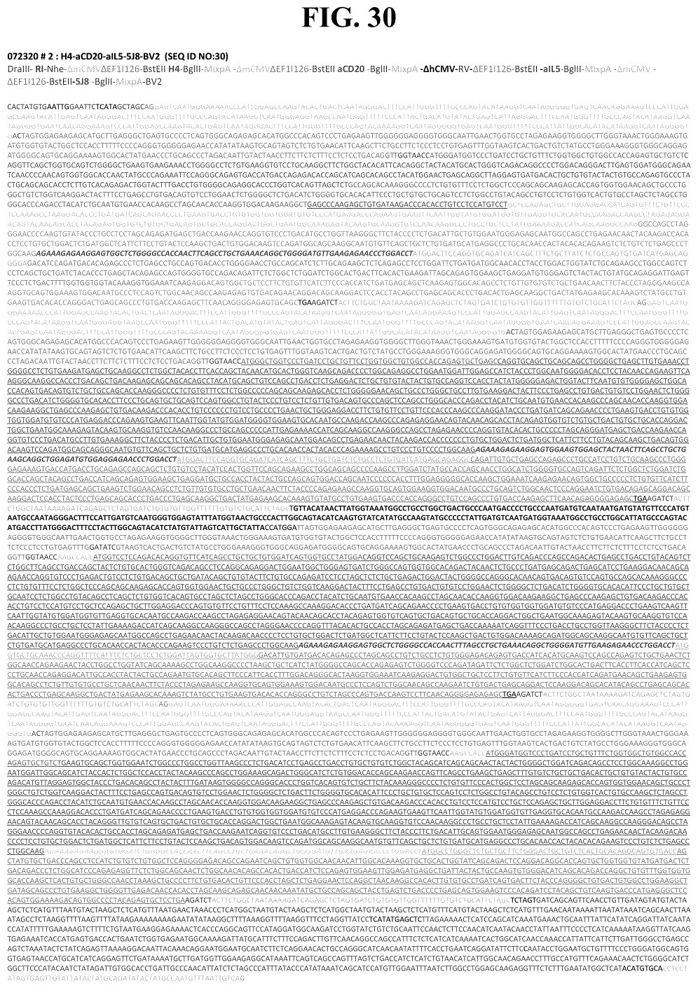

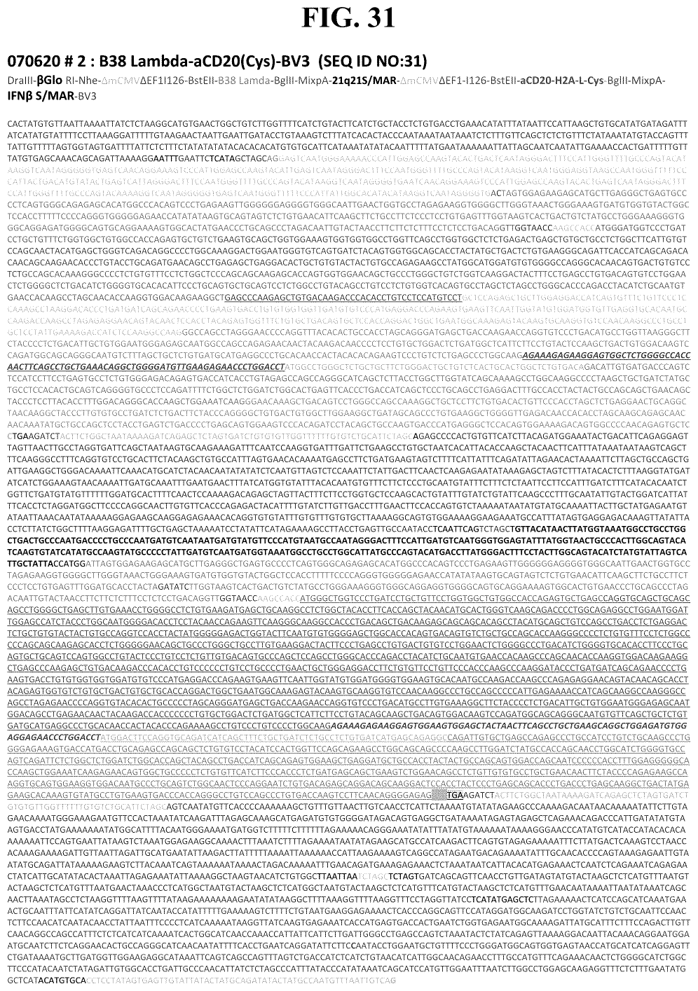

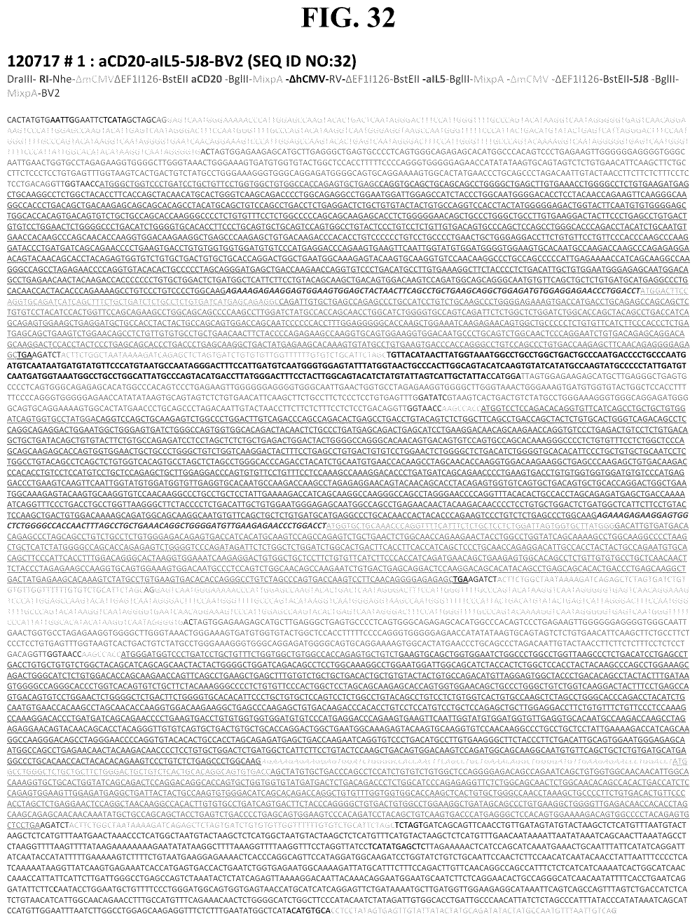

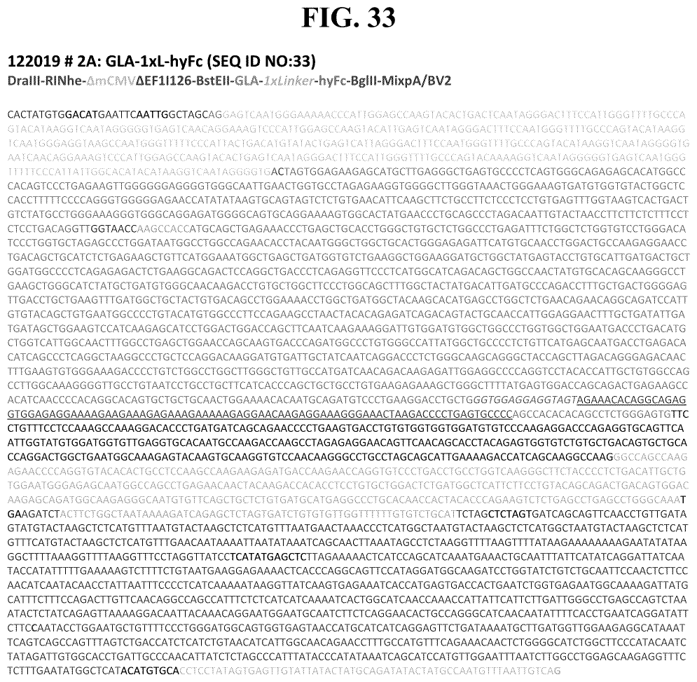

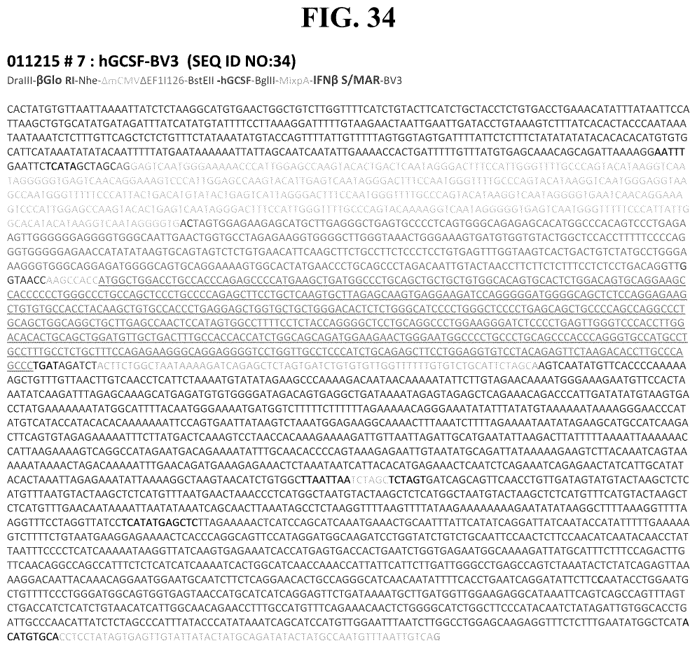

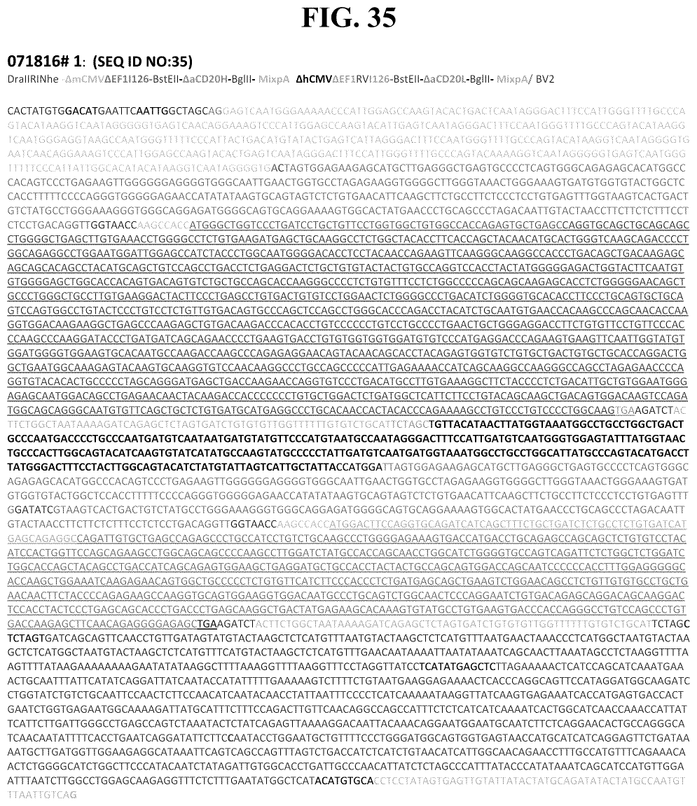

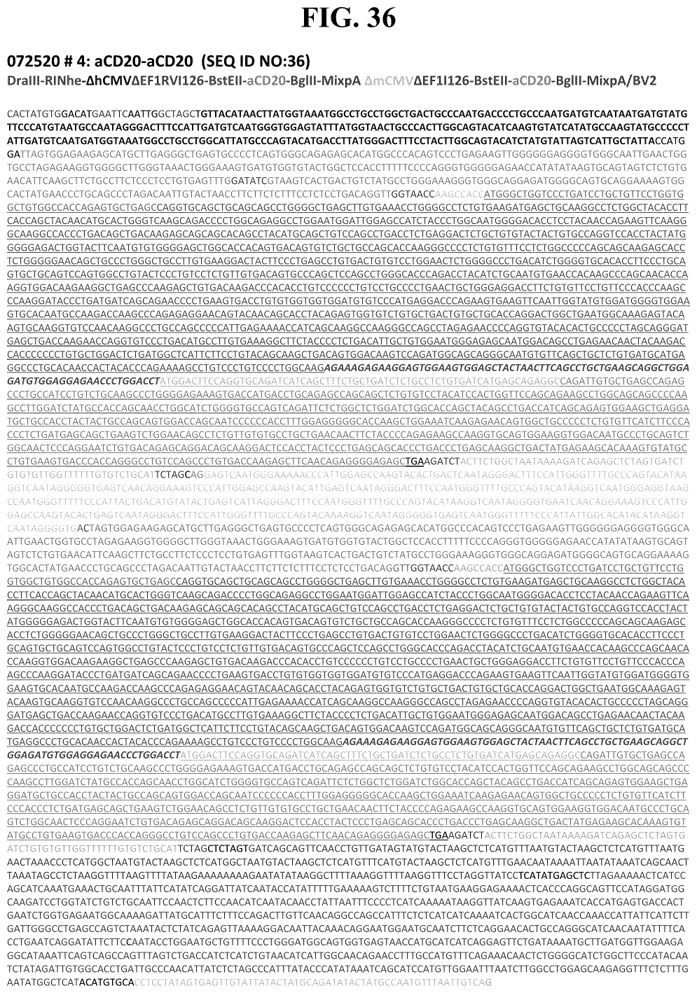

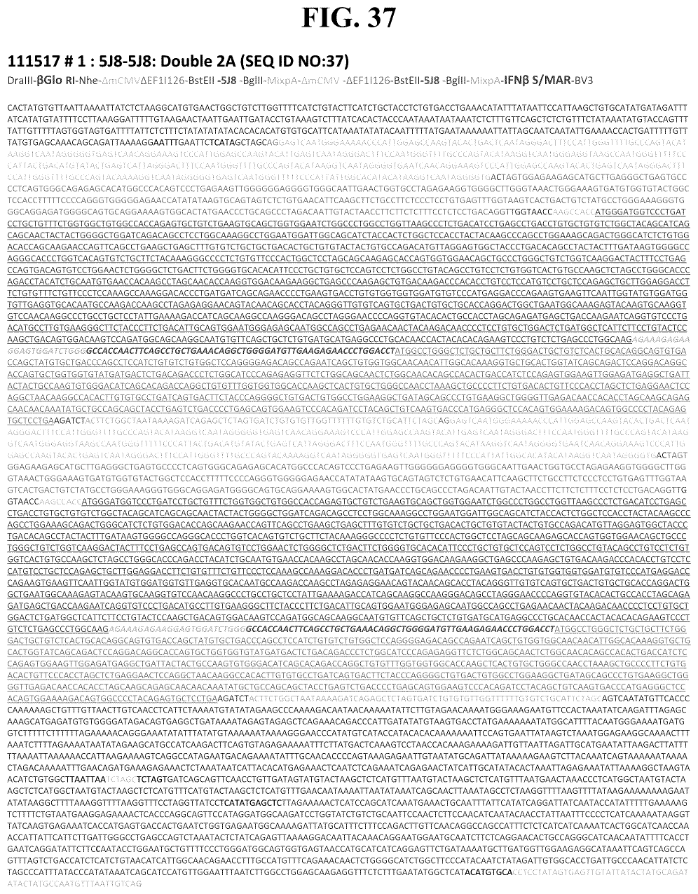

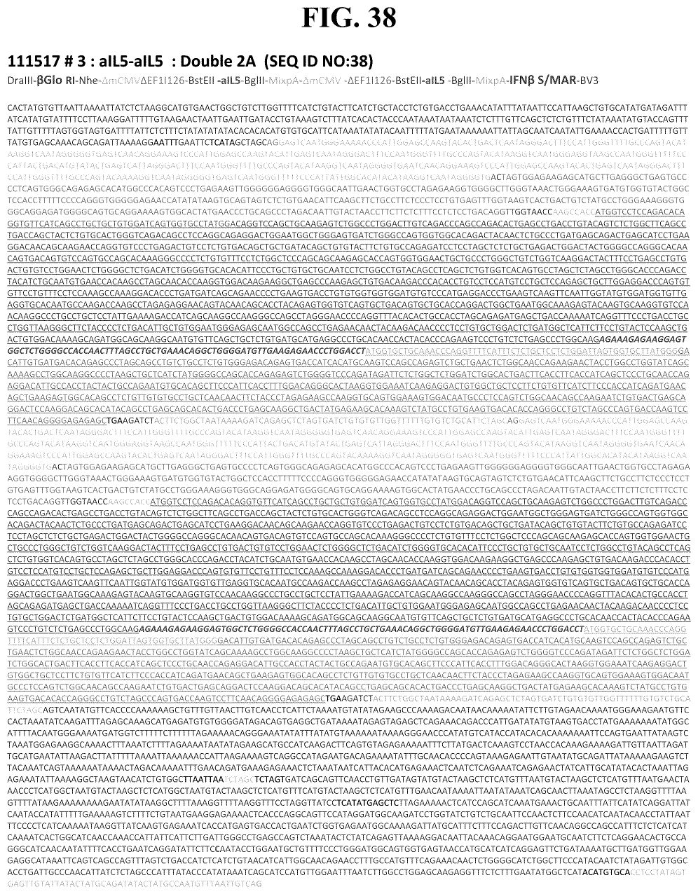

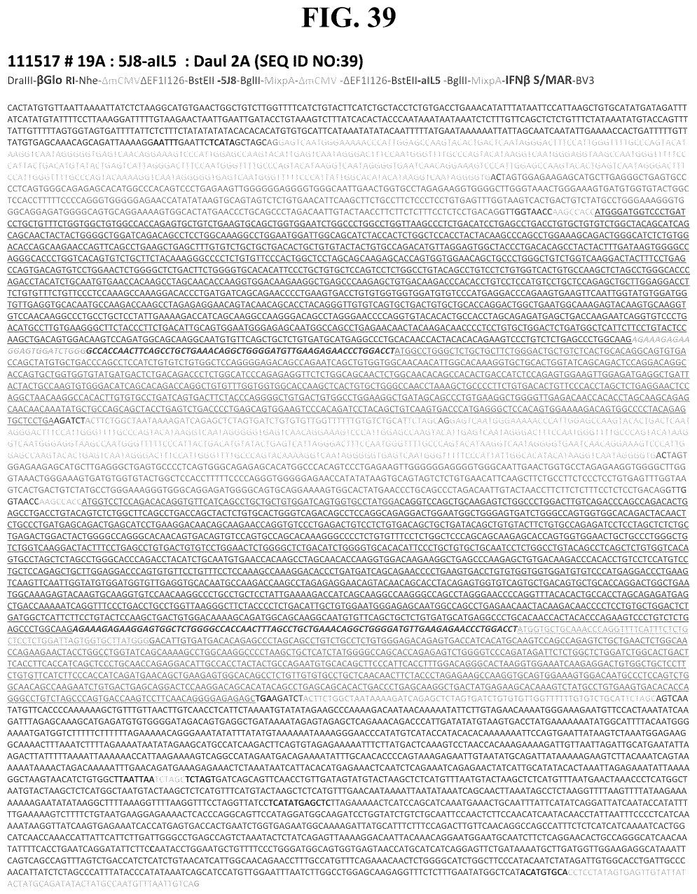

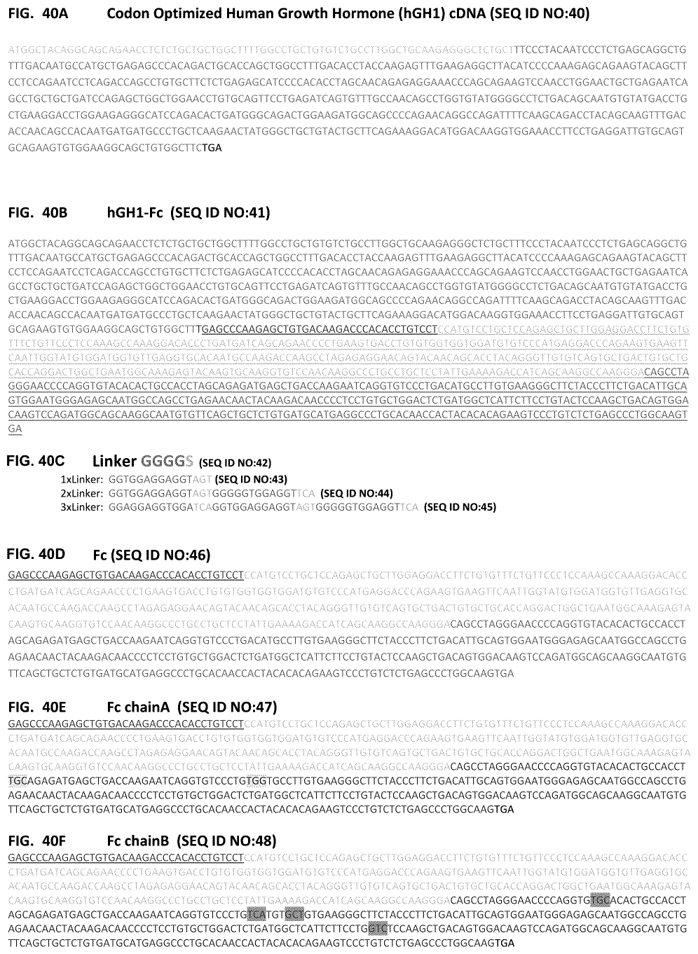

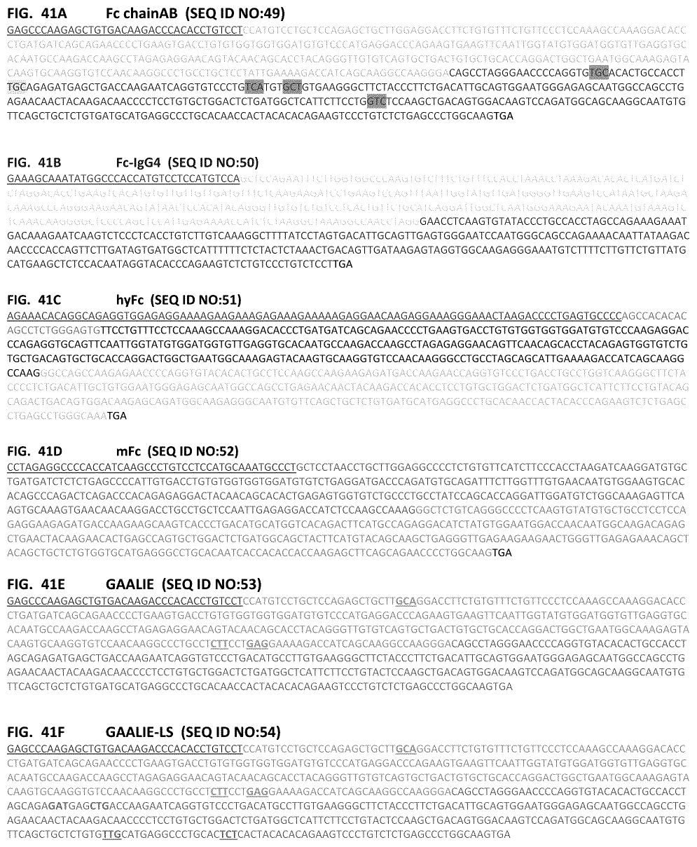

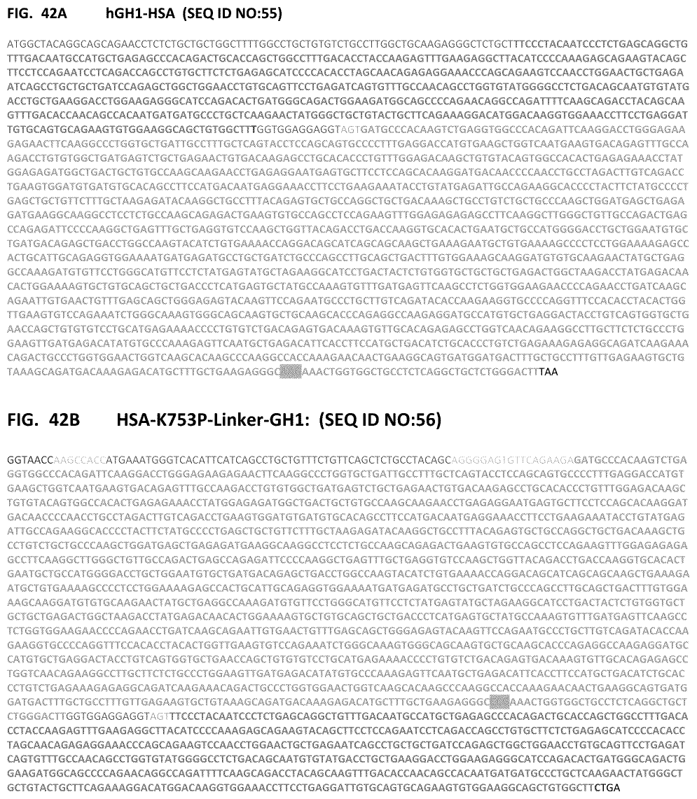

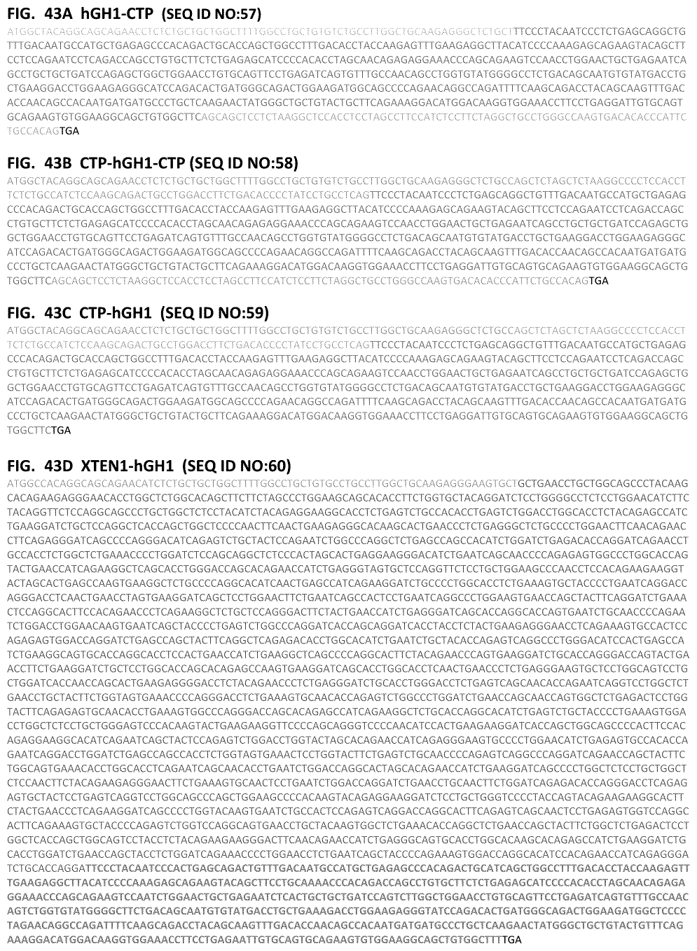

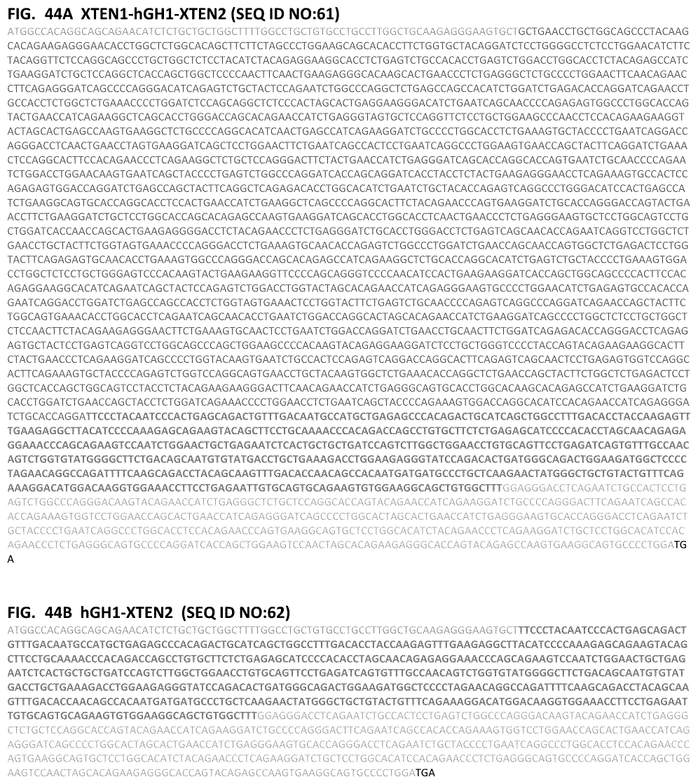

Experimental methods: On day 0, twelve groups of mice, each containing three mice per group, were given IP injections of dexamethasone (40 mg/kg) two hours prior to sequential IV injection of lipids (1000 nmol DOTAP SUV with 2.5 mol % dexamethasone palmitate and 1000 nmol DMPC (1,2-Dimyristoyl-SN-glycero-3-phosphocholine neutral lipid with 5 mol % dexamethasone palmitate), followed by injection of 88 mg of a single pDNA encoding human ACE2 cDNA (Group 1) or a modified version of ACE2, groups 2 thru 12, as indicated. One day later, serum expression of ACE2 was determined by ELISA using recombinant RBD protein for capture, and either an anti-Fc reagent or anti-ACE2 reagent for detection. Group mean+/−SEM expression levels are indicated in FIG. 9, which shows the results.

### Example 10

### Expression of Human Growth Hormone Fused to Half-Life Extending Peptide

This Example describes the in vivo expression of human growth hormone (hGH) fused to a half-life extending peptide.

Methods: Groups of 4 (red) or 3 (other groups) CD-1 mice each were injected with 40 mg/kg water-soluble dexamethasone IP. Two hours later, mice were injected IV, first with liposomes followed approximately 2 minutes later with 75 ug plasmid DNA encoding human GH (hGH). All liposome mixtures contained 1000 nmol DOTAP SUV with 2.5% Dexamethasone 21-Palmitate as well as 1000 nmol DMPC with 5% Dexamethasone 21-palmitate. Mice were bled 24 hours after injection, then weekly or every few weeks thereafter to obtain serum. Serum levels of hGH were assessed by ELISA. At day 127 after injection, serum levels of mouse IGF-1, as well as of hGH were coordinately assessed by their respective ELISAs.

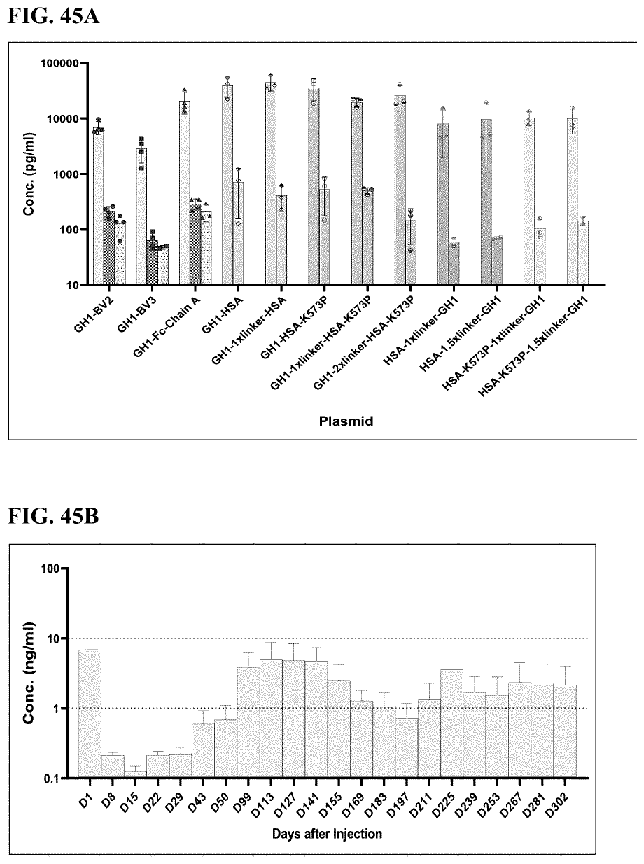

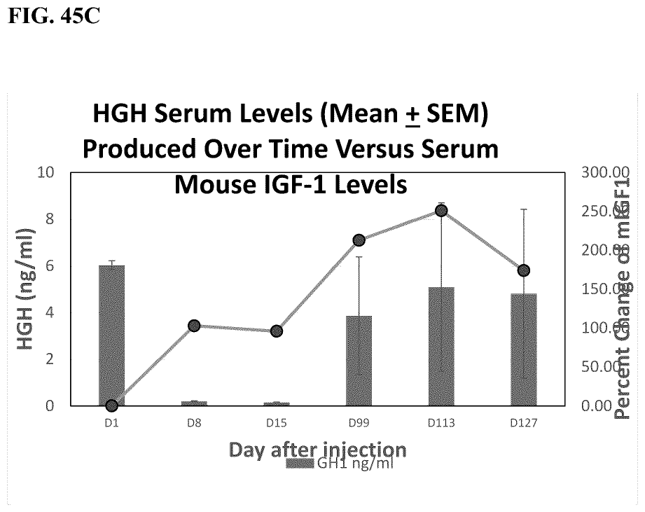

The results are shown in FIGS. 45A-45C. FIG. 45A shows this procedure drives expression of the wild type hGH cDNA fused to a protein half-life extending DNA sequence, including Fc, serum albumin or Xten, and can significantly increase serum hGH levels over time in immunocompetent mice when compared to hGH serum levels produced by a hGH DNA vector that lack protein half-life extending DNA sequences. FIG. 45B shows that the cDNA-encoded hGH protein produced is fully bioactive, as it appropriately increases the levels of the hGH-regulated, endogenous mouse, IGF-1 protein. FIG. 45C shows one injection of a DNA vector in this procedure drives the wild type hGH cDNA but lacking any protein half-life extending DNA sequence can produce durable production of therapeutic hGH serum levels in immunocompetent mice. This is despite the fact that the serum half-life of the hGH protein is less than 20 minutes.

### Example 11

### Expression of Human Growth Hormone Fused to Half-Life Extending Peptide

This Example describes the in vivo expression of human growth hormone (hGH) fused to a half-life extending peptide.

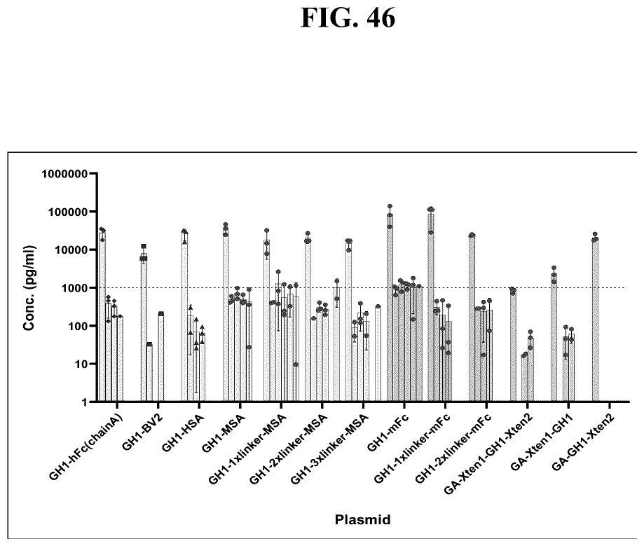

Methods: Groups of 4 CD-1 mice each were injected with 40 mg/kg water-soluble dexamethasone IP. Two hours later, mice were injected IV, first with liposomes followed approximately 2 minutes later with 75 ug plasmid DNA encoding human GH. All liposome mixtures contained 1000 nmol DOTAP SUV with 2.5% Dexamethasone 21-Palmitate as well as 1000 nmol DMPC with 5% Dexamethasone 21-palmitate. Mice were bled 24 hours after injection and every 7-21 days thereafter to isolate serum, and serum expression assessed by ELISA. The results are shown in FIG. 46, which demonstrate that this procedure with vectors driving the wild type hGH cDNA fused to a protein half-life extending DNA sequence, including Fc, serum albumin or Xten, can significantly increase serum hGH levels over time in immunocompetent mice when compared to hGH serum levels produced by a hGH DNA vector that lack protein half-life extending DNA sequences.

### Example 12

### Expression of Human Growth Hormone with Reinjection of Plasmid

This Example describes the in vivo expression of human growth hormone (hGH) with reinjection of the plasmid.

Methods: Groups of 4 CD-1 mice each were injected with 40 mg/kg water-soluble dexamethasone IP. Two hours later, mice were injected IV, first with liposomes followed approximately 2 minutes later with 75 ug plasmid DNA encoding human GH. All liposome mixtures contained 1000 nmol DOTAP SUV with 2.5% Dexamethasone 21-Palmitate as well as 1000 nmol DMPC with 5% Dexamethasone 21-palmitate. Mice were bled weekly to assess expression. Expression for 43 days after initial injection are shown for pre-reinjection. On day 49, mice were given the same treatment as the initial injection. Mice were bled 24 hours after re-injection to isolate serum and every 7-21 days thereafter, and serum expression assessed by ELISA.

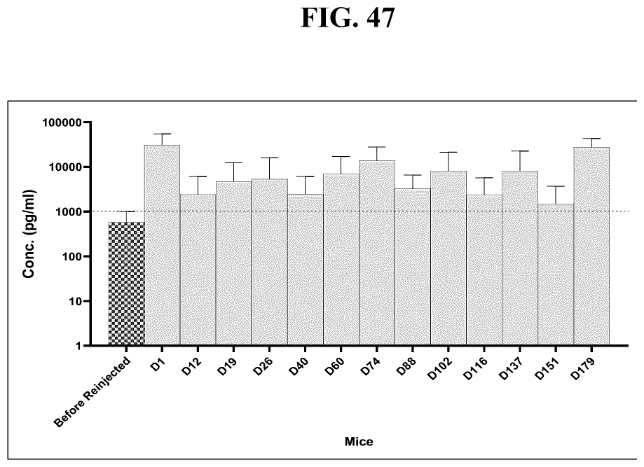

These results are shown in FIG. 47 and demonstrate that, using this procedure, one re-injection of a DNA vector driving the wild type hGH cDNA into fully immunocompetent mice can significantly and durably further increase serum hGH levels produced by the initial hGH DNA vector injection.

### Example 13

### Expression of Human Growth Hormone Fused to Half-Life Extending Peptide

This Example describes the in vivo expression of human growth hormone (hGH) fused to a half-life extending peptide.

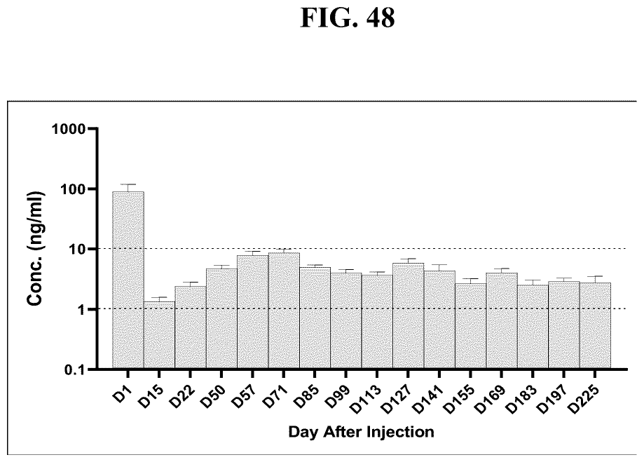

Methods: Groups of 5 CD-1 mice were used. Mice were injected with 40 mg/kg water-soluble dexamethasone IP. Two hours later, mice were injected IV, first with liposomes followed approximately 2 minutes later with 75 ug plasmid DNA encoding human GH. All liposome mixtures contained 1000 nmol DOTAP SUV with 2.5% Dexamethasone 21-Palmitate as well as 1000 nmol DMPC with 5% Dexamethasone 21-palmitate. Mice were bled 24 hours after injection and every 7-28 days thereafter to isolate serum, and serum expression assessed by ELISA. The results are shown in FIG. 48. These results demonstrate that this procedure with DNA vectors driving the wild type hGH cDNA fused to an Fc protein half-life extending DNA sequence can produce serum hGH levels within the 1 to 10 ng/ml hGH therapeutic range for at least the next 225 days (>30% of a normal mouse's lifetime) after a single injection into immunocompetent mice.

### Example 14

### Expression of Human Growth Hormone Fused to Half-Life Extending Peptide

This Example describes the in vivo expression of human growth hormone (hGH) fused to a half-life extending peptide.

Methods: Groups of 3 CD-1 mice each were injected with 40 mg/kg water-soluble dexamethasone IP. Two hours later, mice were injected IV, first with liposomes followed approximately 2 minutes later with 75 ug plasmid DNA encoding human GH. All liposome mixtures contained 1000 nmol DOTAP SUV with 2.5% Dexamethasone 21-Palmitate as well as 1000 nmol DMPC with 5% Dexamethasone 21-palmitate. Mice were bled 24 hours after injection and every 7-21 days thereafter to isolate serum, and serum expression assessed by ELISA.

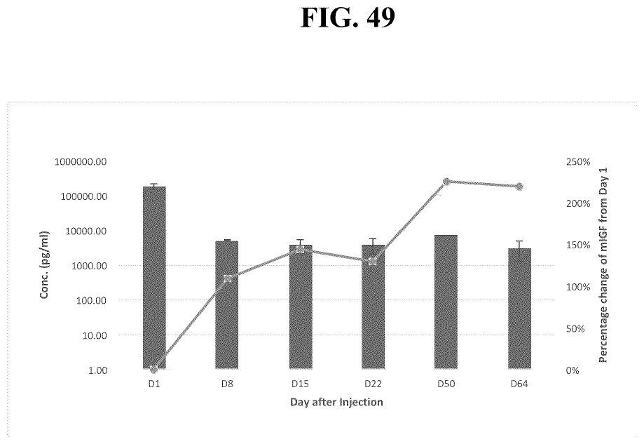

The results are shown in FIG. 49. These results demonstrate this procedure with DNA vectors driving the wild type hGH cDNA fused to an Fc protein half-life extending DNA sequence produce fully bioactive hGH protein in mice, as the cDNA-encoded hGH protein appropriately increases the levels of the hGH-regulated, endogenous mouse, IGF-1 protein.

### Example 15

### Expression of Human Growth Hormone Fused to Half-Life Extending Peptide

This Example describes the in vivo expression of human growth hormone (hGH) fused to a half-life extending peptide.

Methods: Groups of 3 CD-1 mice each were injected with 40 mg/kg water-soluble dexamethasone IP. Two hours later, mice were injected IV, first with liposomes followed approximately 2 minutes later with 75 ug plasmid DNA encoding human GH. All liposome mixtures contained 1000 nmol DOTAP SUV with 2.5% Dexamethasone 21-Palmitate as well as 1000 nmol DMPC with 5% Dexamethasone 21-palmitate. Mice were bled day 1 and day 15 after injection to isolate serum, and serum expression assessed by ELISA.

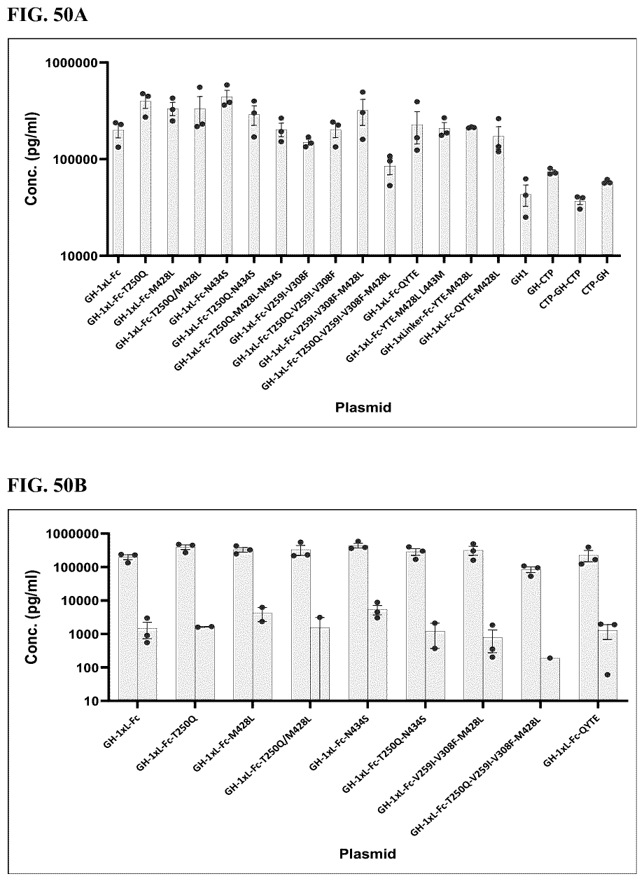

FIGS. 50A-50B show the results. FIG. 50A shows that selective site-directed mutagenesis of the Fc region of an DNA vector driving the wild type hGH cDNA fused to an Fc protein half-life extending DNA sequence including CTP can selectively either increase or decrease serum hGH levels produced in immunocompetent mice. FIG. 50B shows that selective site-directed mutagenesis of the Fc region of a DNA vector driving the wild type hGH cDNA fused to an Fc protein half-life extending DNA sequence can selectively increase serum hGH levels produced over time in immunocompetent mice.

### Example 16

### Immuno-modulation Agents

This Example describes the testing of various immuno-modulating agents.

### Part 1

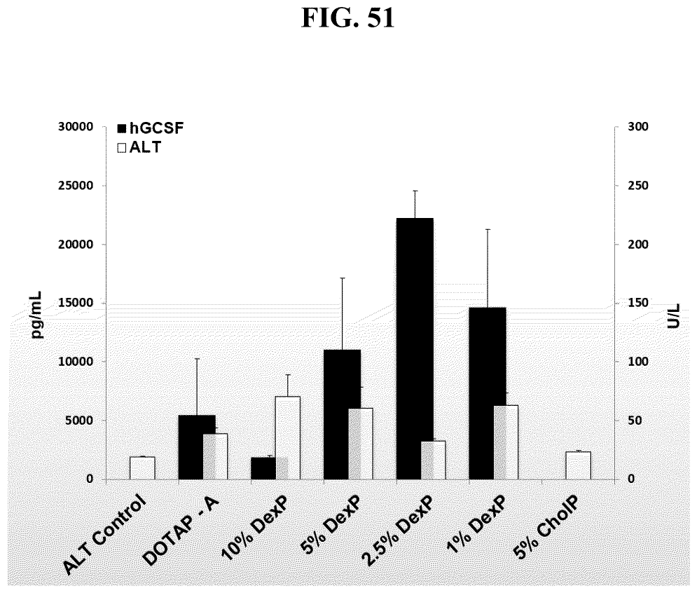

Methods: Groups of 3 CD-1 mice each were injected with 900 nmol DOTAP SUV, with or without Dexamethasone 21-palmitate or Cholesteryl palmitate in molar percentages as shown in FIG. 51. Two minutes after liposome injection, mice were injected with 70 ug plasmid DNA encoding hG-CSF. Mice were bled the following day and serum levels of hG-CSF protein was assessed by ELISA. ALT levels were assessed in sera. Results are shown in FIG. 51, which shows that incorporating an optimized molar percentage of dexamethasone palmitate (DexPalm) into cationic liposomes can both further increase gene expression and further decrease toxicity.

### Part 2

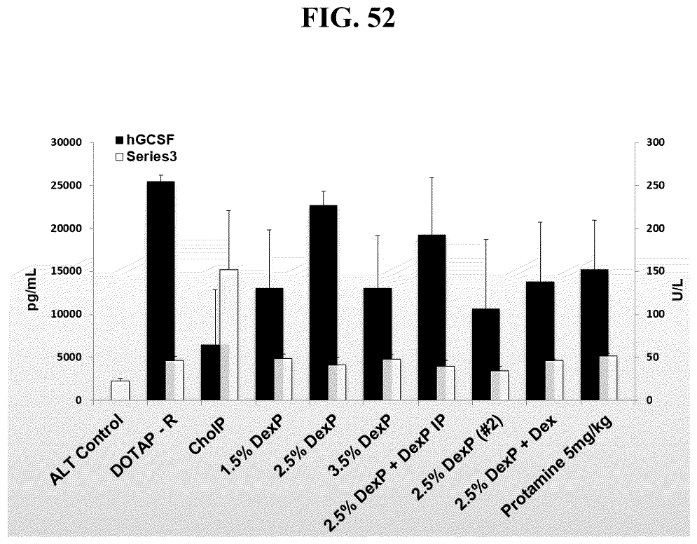

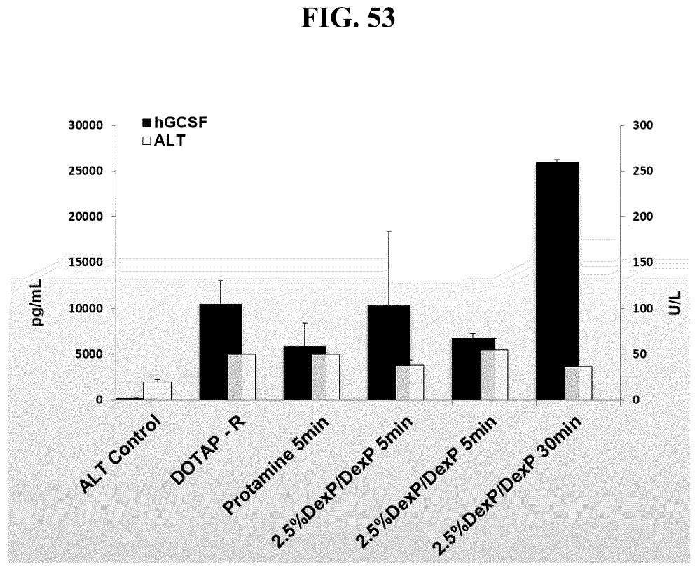

Methods: Groups of 3 CD-1 mice each were used. One group (+Dex) was injected IP with 40 mg/kg Dexamethasone, one group (+DexP IP) was injected IP with 900 nmol DOTAP liposomes containing 2.5 molar % Dexamethasone 21-palmitate, and one group (Protamine) was injected IP with 5 mg/kg Protamine sulfate. Two hours later, mice were first injected with 900 nmol DOTAP SUV, with or without Dexamethasone 21-palmitate or Cholesteryl palmitate in molar percentages as shown in FIG. 52. Two minutes after liposome injection, mice were injected with 70 ug plasmid DNA encoding hG-CSF. Mice were bled the following day and serum levels of hG-CSF protein was assessed by ELISA. ALT levels were assessed in sera. The results are shown in FIG. 52, which show that incorporating an optimized molar percentage of dexamethasone palmitate into cationic liposomes can both further increase gene expression and further decrease toxicity.

### Part 3

Methods: Groups of 3 CD-1 mice each were used. One group each was injected IP with 900 nmol DOTAP liposomes containing 2.5% Dexamethasone 21-palmitate, 5 minutes before, 5 minutes after, or 30 minutes before IV injections. One group was and one group (Protamine) was injected IP with 5 mg/kg Protamine sulfate 5 minutes before IV injections. For IV injections, mice were first injected with 900 nmol DOTAP SUV with 2.5% Dexamethasone 21-palmitate in the liposomes. Two minutes after liposome injection, mice were injected with 70 ug plasmid DNA encoding hG-CSF. Mice were bled the following day and serum levels of hG-CSF protein was assessed by ELISA. ALT levels were assessed in sera. FIG. 53 shows the results, which show that pre-injecting an optimized molar percentage of dexamethasone palmitate in liposomes prior to injecting cationic liposomes can both further increase gene expression and further decrease toxicity.

### Part 4

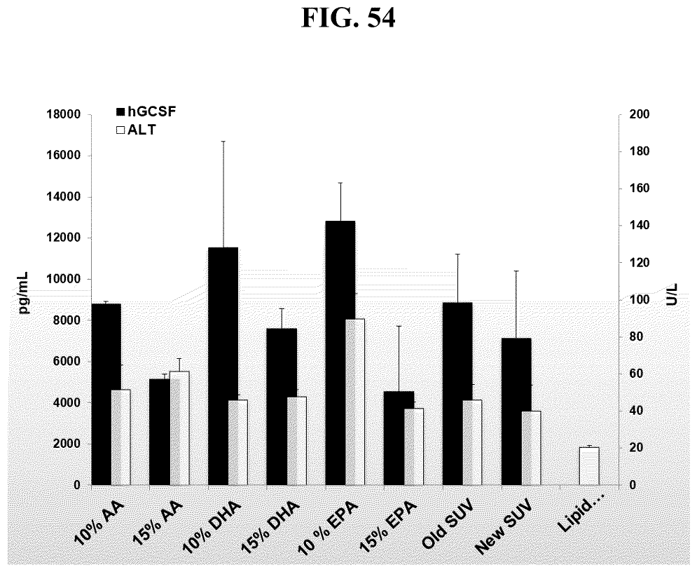

Methods: Groups of 3 CD-1 mice each were injected with 900 nmol DOTAP SUV, with or without one of a number of different endogenous, anti-inflammatory lipids (AILs) in molar percentages in the liposomes as shown in FIG. 54. Two minutes after liposome injection, mice were injected with 70 ug plasmid DNA encoding hG-CSF. Mice were bled the following day and serum levels of hG-CSF protein was assessed by ELISA. ALT levels were assessed in sera. The results are shown in FIG. 54, which shows that injecting some AILs incorporated into cationic liposomes can both further increase gene expression and further decrease toxicity (ALT levels). In contrast, injecting selected molar percentages of other AILs incorporated into cationic liposomes can significantly increase ALT.

### Part 5

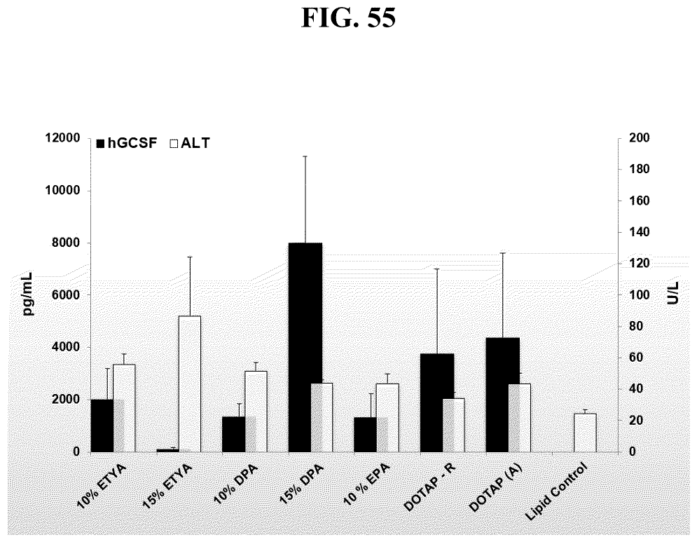

Methods: Groups of 3 CD-1 mice each were injected with 900 nmol DOTAP SUV, with or without one of a number of different endogenous, anti-inflammatory lipids (AILs) in molar percentages as shown in FIG. 55. Two minutes after liposome injection, mice were injected with 70 ug plasmid DNA encoding hG-CSF. Mice were bled the following day and serum levels of hG-CSF protein was assessed by ELISA. ALT levels were assessed in sera. The results, shown in FIG. 55, show that injecting certain ATLs incorporated into cationic liposomes can both further increase gene expression and further decrease toxicity (ALT levels). In contrast, injecting selected molar percentages of other AILs incorporated into cationic liposomes can significantly increase ALT.

### Part 6

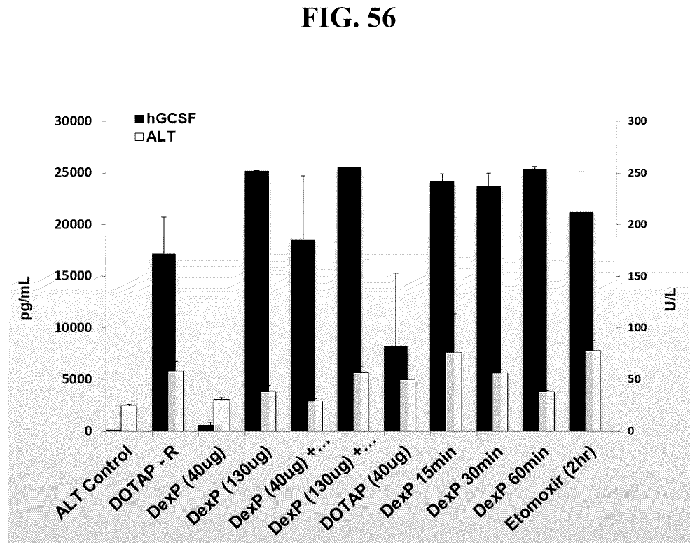

Methods: Groups of 3 CD-1 mice each were used. One group (+Dex) was injected IP with 40 mg/kg Dexamethasone, one group. Two hours later, mice were first injected with 900 nmol DOTAP SUV, with or without 5 mole percent Dexamethasone 21-palmitate. Two minutes after liposome injection, mice were injected with either 40 or 130 ug plasmid DNA encoding hG-CSF. Mice were bled the following day and serum levels of hG-CSF protein was assessed by ELISA. ALT levels were assessed in sera. The results are shown in FIG. 56, and show that incorporating an optimized molar percentage of dexamethasone palmitate into cationic liposomes can further increase peak levels of gene expression following an otherwise ineffective hG-CSF-DNA dose.

### Example 17

### Intranasal Administration and Immunomodulation

This Example describes targeting hematopoietic cells in mouse lungs following Intranasal administration of liposomes.

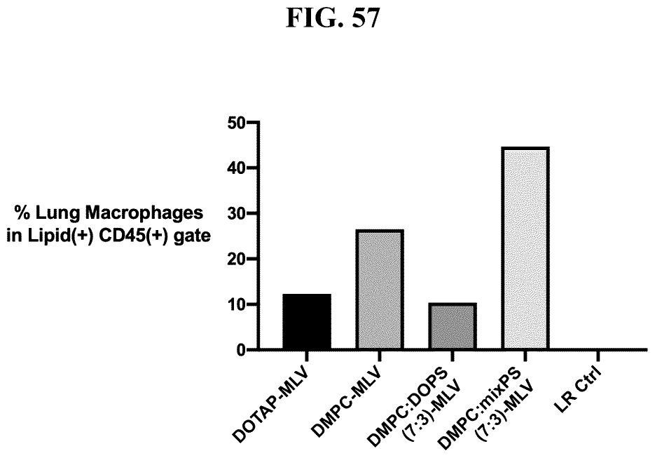

Experimental Methods: Mice were anesthetized and administered via intranasal route 200 nmol of the indicated liposome formulations each containing 1 mol % fluorescent phosphatidyl-ethanolamine to track uptake of liposomes or lactated ringers control. One day later, lungs were harvested, digested to single cell suspensions and surface stained with fluorescent antibodies to detect mouse CD45, CD11b and F4/80 markers prior to analysis by flow cytometry. DOPS=1,2-dioleoyl-sn-glycero-3-phospho-L-serine, mixPS=1-stearoyl-2-oleoyl-sn-glycero-3-phospho-L-serine. The results are shown in FIG. 57, which shows that by selectively modifying the lipid composition of liposomes administered intranasally, that these liposomes can be selectively targeted to intrapulmonary monocytes and macrophages to different extents, thus selectively immune-modulating the lung.

### Example 18

### Differential T Cell Activation

This Example describes differential T cell activation resulting from administration of particular liposome formulations.

Experimental Methods: On day 0, six groups of mice, each containing three mice per group, were given the following treatments: Group 1—IP injection of dexamethasone (40 mg/kg) two hours prior to sequential IV injection of lipids (1000 nmol DOTAP SUV with 2.5 mol % dexamethasone palmitate and 1000 nmol DMPC (1,2-Dimyristoyl-SN-glycero-3-phosphocholine neutral lipid) with 5 mol % dexamethasone palmitate), followed by a single pDNA encoding anti-SARS CoV2 H4 kappa mAb, anti-CD20, anti-influenza A 5J8, and anti-human IL-5.

Group 2—Sequential IV injection of lipids (1000 nmol DOTAP SUV with 2.5 mol % dexamethasone palmitate and 1000 nmol DMPC (1,2-Dimyristoyl-SN-glycero-3-phosphocholine neutral lipid) with 5 mol % dexamethasone palmitate), followed by a single pDNA encoding anti-SARS CoV2 H4 kappa mAb, anti-CD20, anti-influenza A 5J8, and anti-human IL-5.

Group 3—Sequential IV injection of lipids (1000 nmol DOTAP SUV and 1000 nmol DMPC (1,2-Dimyristoyl-SN-glycero-3-phosphocholine neutral lipid) with 5 mol % dexamethasone palmitate), followed by a single pDNA encoding anti-SARS CoV2 H4 kappa mAb, anti-CD20, anti-influenza A 5J8, and anti-human IL-5.

Group 4—Sequential IV injection of lipids (1000 nmol DOTAP SUV with 2.5 mol % dexamethasone palmitate and 1000 nmol DMPC (1,2-Dimyristoyl-SN-glycero-3-phosphocholine neutral lipid), followed by a single pDNA encoding anti-SARS CoV2 H4 kappa mAb, anti-CD20, anti-influenza A 5J8, and anti-human IL-5.

Group 5—Sequential IV injection of lipids (1000 nmol DOTAP SUV with 2.5 mol % dexamethasone palmitate and 1000 nmol DMPC (1,2-Dimyristoyl-SN-glycero-3-phosphocholine neutral lipid) with 5 mol % dexamethasone palmitate), followed by a single pDNA encoding anti-SARS CoV2 H4 kappa mAb, anti-CD20, anti-influenza A 5J8, and anti-human IL-5.

Group 6—No Treatment

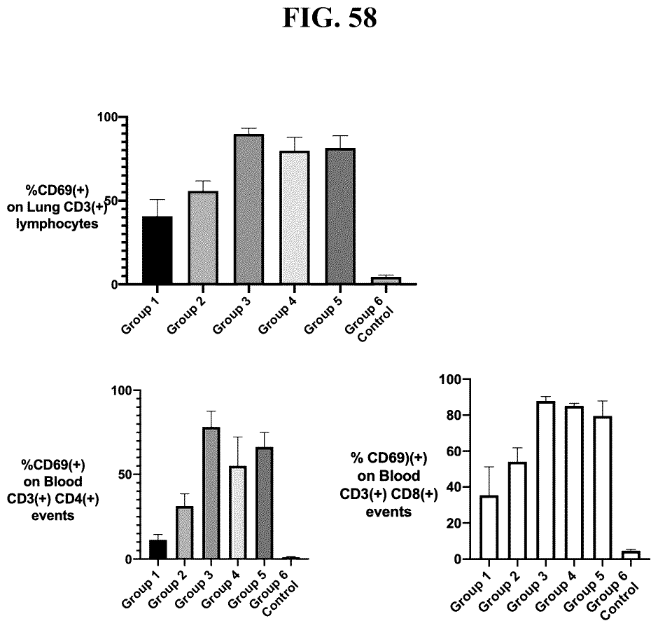

FIG. 58 shows the results and shows that by selectively modifying a parenteral aqueous soluble predose, and/or the molar percentage of dexamethasone palmitate incorporated into subsequently administered liposomes, that the level of T lymphocyte activation both in lung and in the blood can be selectively immuno-modulated.

### Example 19

### Differential T Cell Activation

This Example describes differential T cell activation resulting from administration of liposome formulations.

Experimental Methods: On day 0, eight groups of mice, each containing three mice per group, were treated as follows:

Group 1—Untreated

Group 2—IP injection of dexamethasone (40 mg/kg) two hours prior to sequential IV injection of lipids (1000 nmol DOTAP SUV with 2.5 mol % dexamethasone palmitate and 1000 nmol DMPC (1,2-Dimyristoyl-SN-glycero-3-phosphocholine neutral lipid) with 5 mol % dexamethasone palmitate), followed by a single pDNA encoding human PECAM-1.

Group 3—IP injections of dexamethasone (40 mg/kg) two hours prior to sequential IV injection of lipids (1000 nmol DOTAP:cholesterol (85:15) SUV with 2.5 mol % dexamethasone palmitate and 1000 nmol DMPC (1,2-Dimyristoyl-SN-glycero-3-phosphocholine neutral lipid) with 5 mol % dexamethasone palmitate), followed by a single pDNA encoding human PECAM-1.

Group 4—IP injections of dexamethasone (40 mg/kg) two hours prior to sequential IV injection of lipids (1000 nmol DOTAP:DODAP (1:1) SUV with 2.5 mol % dexamethasone palmitate and 1000 nmol DMPC (1,2-Dimyristoyl-SN-glycero-3-phosphocholine neutral lipid) with 5 mol % dexamethasone palmitate), followed by a single pDNA encoding human PECAM-1.

Group 5—IP injections of dexamethasone (40 mg/kg) two hours prior to sequential IV injection of lipids (1000 nmol DOTAP SUV with 2.5 mol % dexamethasone palmitate and 1000 nmol DMPC (1,2-Dimyristoyl-SN-glycero-3-phosphocholine neutral lipid):cholesterol (1:1) with 5 mol % dexamethasone palmitate), followed by a single pDNA encoding human PECAM-1.

Group 6—Two IP injections of dexamethasone (40 mg/kg) two hours prior and just prior to sequential IV injection of lipids (1000 nmol DOTAP SUV with 2.5 mol % dexamethasone palmitate and 1000 nmol DMPC (1,2-Dimyristoyl-SN-glycero-3-phosphocholine neutral lipid):cholesterol (1:1) with 5 mol % dexamethasone palmitate), followed by a single pDNA encoding human PECAM-1.

Group 7—Two IP injections of 2.5 mol % dexamethasone palmitate in phosphatidylserine:cholesterol 2:1 MLV 24 hours and two hours prior to sequential IV injection of lipids (1000 nmol DOTAP SUV with 2.5 mol % dexamethasone palmitate and 1000 nmol DMPC (1,2-Dimyristoyl-SN-glycero-3-phosphocholine neutral lipid) with 5 mol % dexamethasone palmitate), followed by a single pDNA encoding human PECAM-1.

Group 8—Two IP injections of 2.5 mol % dexamethasone palmitate in DOTAP:cholesterol 2:1 MLV 24 hours and two hours prior to sequential IV injection of lipids (1000 nmol DOTAP SUV with 2.5 mol % dexamethasone palmitate and 1000 nmol DMPC (1,2-Dimyristoyl-SN-glycero-3-phosphocholine neutral lipid) with 5 mol % dexamethasone palmitate), followed by a single pDNA encoding human PECAM-1.

One day later, lungs and peripheral blood were harvested, digested to single cell suspensions if necessary, and surface stained with fluorescent antibodies to detect mouse CD4, CD8 alpha, CD44, CD69, and human PECAM-1 markers prior to analysis by flow cytometry.

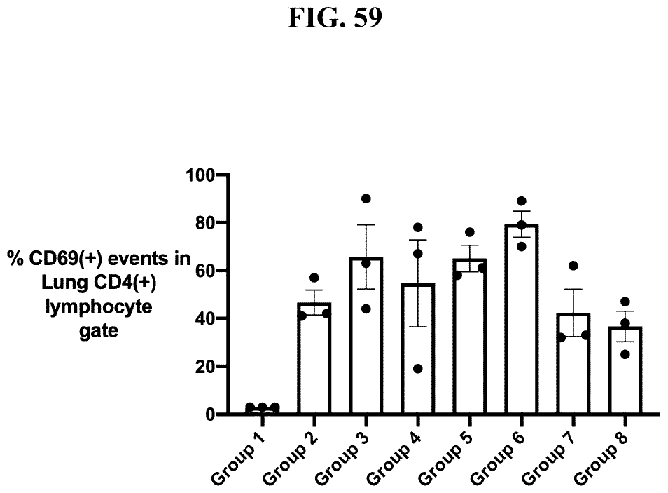

FIG. 59 shows the results, which show that by selectively modifying a parenteral aqueous soluble pre-dose, and/or the molar percentage of dexamethasone palmitate incorporated into subsequently administered liposomes, that the level of T lymphocyte activation both in lung and in the blood can be selectively immuno-modulated.

### Example 20

### Anti-TNFa and Heparinoid Agents

This Example describes the use of anti-TNFa monoclonal antibodies and Heparinoid Agents for increasing expressing in in vivo expression methods.

### Part 1—Anti-TNFa Monoclonal Antibody

Methods: Groups of 3 mice were used. One group was given 100 ug each anti-TNFa monoclonal antibody per mouse IP, 2 hours prior to IV injections. Mice were then injected IV with 900 nmol DOTAP SUV, followed 2 minutes later by either 70 ug or 130 ug plasmid DNA encoding hG-CSF. Mice were bled 24 hours after injection, and hG-CSF expression in the sera assessed by ELISA. Serum ALT/AST levels were measured.

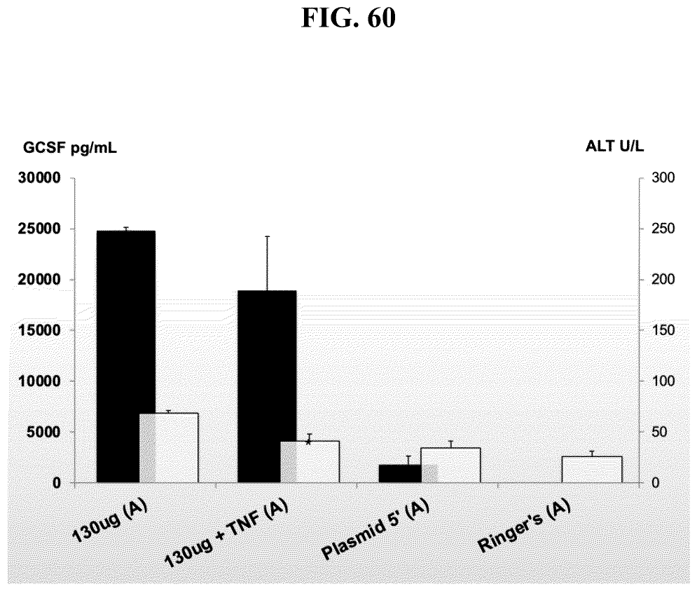

Results are shown in FIG. 60, which shows that pre-administration of an anti-inflammatory agent, here anti-TNF monoclonal antibody, can both further increase gene expression while further reducing its toxicity.

### Part 2—Nsh

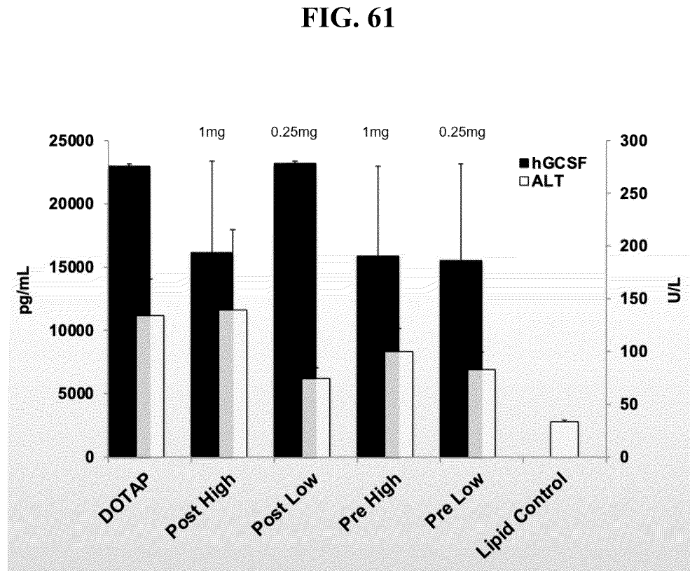

Methods: Groups of 3 mice were used. Except for the control group, mice were given NSH (N-Acetyl-De-O-Sulfated Heparin) IP at 0.25 or 1 mg per mouse either 2 hours pre or 2 hours post lipid and DNA injection. Mice were then injected IV with 900 nmol DOTAP SUV, followed 2 minutes later by 70 ug plasmid DNA encoding hG-CSF. Mice were bled 24 hours after injection, and hG-CSF expression in the sera assessed by ELISA. Serum ALT/AST levels were measured. Results are shown in FIG. 61, which shows that either pre- or post-administration of a NSH can reduce toxicity.

### Part 3—Nsh

Methods: Groups of 3 mice were used. Heparinoid-treated mice were given NSH (N-Acetyl-De-O-Sulfated Heparin) IP at 0.25 or 1 mg per mouse either 2 hours pre or 2 hours post lipid and DNA injection. Mice were then injected IV with 900 nmol DOTAP SUV, followed 2 minutes later by 70 ug plasmid DNA encoding hG-CSF. Mice were bled 24 hours after injection, and hG-CSF expression in the sera assessed by ELISA. Tocopherol-treated mice were given 900 nmol DOTAP SUV containing alpha-tocopherol, followed by 70 ug plasmid DNA encoding hG-CSF. Serum ALT/AST levels were measured.

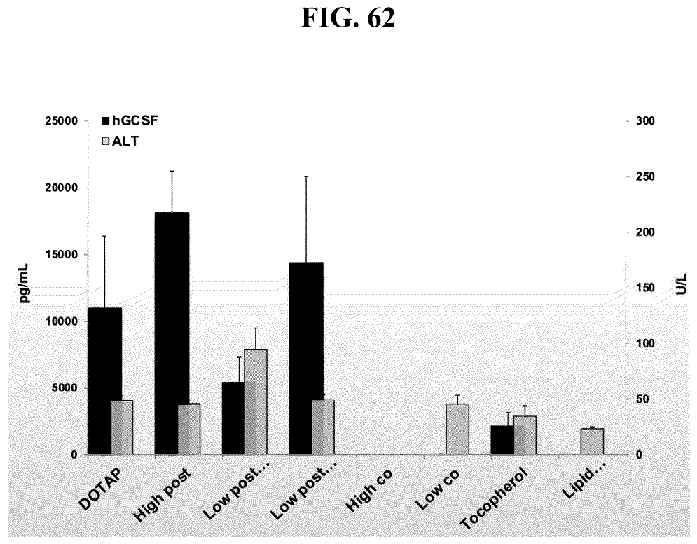

The results are shown in FIG. 62, which show that either pre- or post-administration of a NSH can reduce toxicity.

### Part 4—Nsh

Methods: Groups of 3 mice were used. Heparinoid-treated mice were given NSH (N-Acetyl-De-O-Sulfated Heparin) IP 2 hours prior to lipid and DNA injection. Mice were then injected IV with 900 nmol DOTAP SUV, followed 2 minutes later by 70 ug plasmid DNA encoding hG-CSF. Mice were bled 24 hours after injection, and hG-CSF expression in the sera assessed by ELISA. Tocopherol mice were given 900 nmol DOTAP SUV containing alpha-tocopherol, followed by 70 ug plasmid DNA encoding hG-CSF. Serum ALT/AST levels were measured.

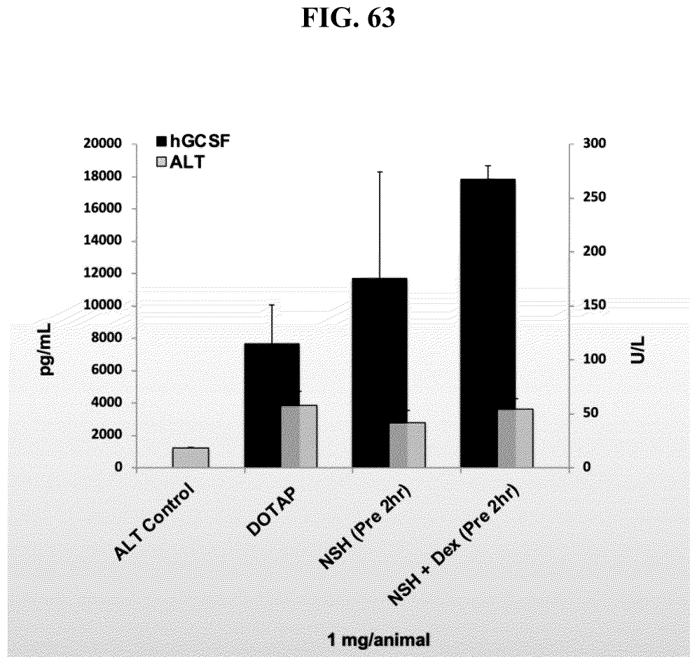

FIG. 63 shows the results which show that either pre-administration of NSH can both further increase gene expression while further reducing its toxicity.

### Example 21

### Immunomodulation Following Liposome Administration

This example describes immunomodulation of the lymphocyte and monocyte cell populations in mice following administration of various liposome formulations containing dexamethasone and/or dexamethasone palmitate.

Experimental Methods: Groups of 2-3 CD-1 mice were used. On day 0, eight groups of mice, were given the following treatments:

Group 1—IP injection of water-soluble dexamethasone (40 mg/kg) only.
Group 2—IP injection of dexamethasone (40 mg/kg) two hours prior to IV injection of lipids (1000 nmol DOTAP SUV with 2.5 mol % dexamethasone palmitate and 1000 nmol DMPC (1,2-Dimyristoyl-SN-glycero-3-phosphocholine neutral lipid) with 5 mol % dexamethasone palmitate).
Group 3—IV injection of lipids (1000 nmol DOTAP SUV with 2.5 mol % dexamethasone palmitate and 1000 nmol DMPC (1,2-Dimyristoyl-SN-glycero-3-phosphocholine neutral lipid) with 5 mol % dexamethasone palmitate).
Group 4—IP injection of 1000 nmol DMPC (1,2-Dimyristoyl-SN-glycero-3-phosphocholine neutral lipid) with 5 mol % dexamethasone palmitate) MLV two hours prior to IV injection of lipids (1000 nmol DOTAP SUV with 2.5 mol % dexamethasone palmitate and 1000 nmol DMPC (1,2-Dimyristoyl-SN-glycero-3-phosphocholine neutral lipid) MLV with 5 mol % dexamethasone palmitate.
Group 5—IP injection of 1000 nmol DMPC (1,2-Dimyristoyl-SN-glycero-3-phosphocholine neutral lipid) with 5 mol % dexamethasone palmitate) SUV two hours prior to IV injection of lipids (1000 nmol DOTAP SUV with 2.5 mol % dexamethasone palmitate and 1000 nmol DMPC (1,2-Dimyristoyl-SN-glycero-3-phosphocholine neutral lipid) MLV with 5 mol % dexamethasone palmitate.
Group 6—IP injection of 1000 nmol DMPC (1,2-Dimyristoyl-SN-glycero-3-phosphocholine neutral lipid) with 5 mol % dexamethasone palmitate) MLV two hours prior to IV injection of lipids (1000 nmol DOTAP SUV with 2.5 mol % dexamethasone palmitate and 1000 nmol DMPC (1,2-Dimyristoyl-SN-glycero-3-phosphocholine neutral lipid) MLV with 5 mol % dexamethasone palmitate containing MTAS-NLS-SPD peptide.
Group 7—IP injection of 1000 nmol DMPC (1,2-Dimyristoyl-SN-glycero-3-phosphocholine neutral lipid) with 5 mol % dexamethasone palmitate) SUV two hours prior to IV injection of lipids (1000 nmol DOTAP SUV with 2.5 mol % dexamethasone palmitate and 1000 nmol DMPC (1,2-Dimyristoyl-SN-glycero-3-phosphocholine neutral lipid) MLV with 5 mol % dexamethasone palmitate containing MTAS-NLS-SPD peptide.
Group 8—No treatment.

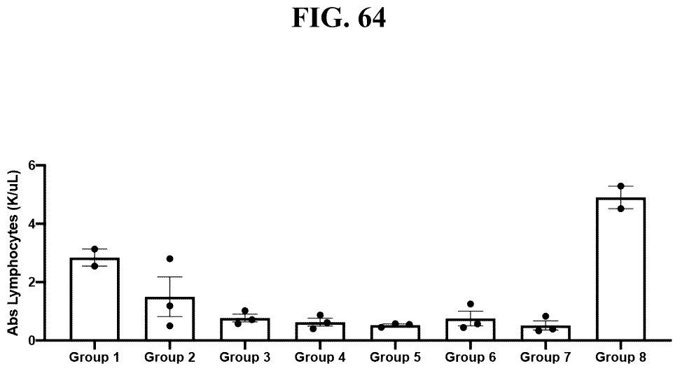

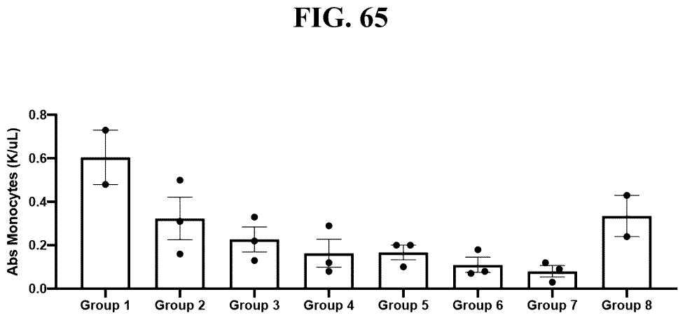

Twenty four hours following liposome treatment, peripheral blood was harvested in EDTA containing microtainer tubes and analyzed by CBC apparatus. Group mean values+/−SEM are displayed. FIG. 64 shows that administration of various formulations of liposomes containing dexamethasone palmitate decreases lymphocyte counts in blood compared to systemic administration of dexamethasone alone. FIG. 65 shows that administration of various formulations of liposomes containing dexamethasone palmitate decreases monocyte counts in blood compared to systemic administration of dexamethasone alone.

### Example 22

### Production of Ongoing Fully SARS-CoV-2 Neutralizing Levels of a Single Anti-SARS-CoV2 mAb Following a Single HEDGES DNA Vector Administration

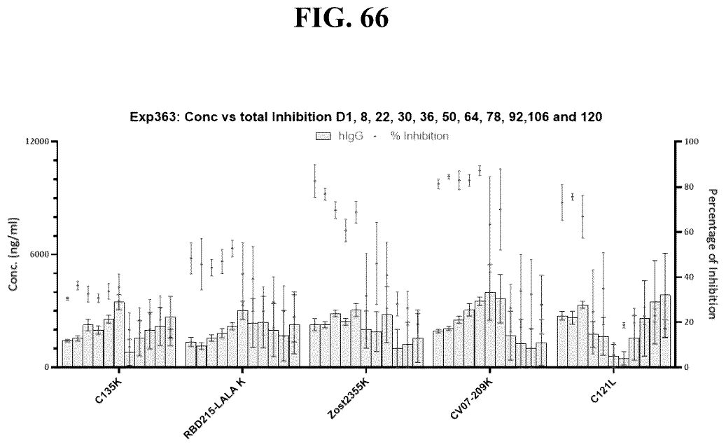

This example describes expression of single SARS-CoV-2 antibodies in mice produces fully neutralizing levels of mAb using the following injection protocol. The five different SARS-CoV-2 antibodies individually expressed in mice were: C135, C215, COV2-2355, CV07-209, and C121 (see Table 7 for sequence information). At day 0, groups of mice were pretreated with 40 mg/kg water-soluble dexamethasone i.p. two hours prior to dosing i.v. with liposomes composing 1100 nmol each of DOTAP/2.5 mol % dexamethasone palmitate/SUV and DMPC/5 mol % dexamethasone palmitate/MLV. After two minutes, mice were dosed i.v. with about 80 ug of a single plasmid DNA containing one expression cassette for one of the five SARS-CoV2-specific mAbs. Mice were bled at days 1, 8, 22, 30, 36, 50, 78, 92, 106, and 120 after treatment and serum mAb protein levels were determined by a human IgG ELISA assay. Results are shown in FIG. 66 (left axis, pink bar graphs represent mean + or −SEM shown in ascending order from day 1 to day 120 for each mAb). The functional bioactivity of SARS-CoV2-specific mAb containing sera to inhibit SARS-CoV2 spike-human ACE2 protein interactions was determined by a commercially-available in vitro SARS-CoV2 spike/ACE2 blocking assay (cPASS, right axis, green dots represent mean + or −SEM shown in ascending order from day 1 to day 120 for each mAb clone, Genscript).

This example demonstrates, as shown in FIG. 66, that one injection of different single DNA expression plasmids each encoding one of five different SARS-CoV2-specific mAb produces fully neutralizing serum levels of each SARS-CoV2-specific mAb for the full experimental course of at least 120 days following administration, and that these ongoing serum mAb levels functionally and continuously block SARS-CoV2 spike-human ACE2 binding for at least 120 days (which is the human equivalent of greater than 20 years). These results demonstrate that this protocol, which includes a DNA injection encoding a single SARS-CoV2-specific mAb, can produce durable (greater than 20 human years equivalence) of neutralizing anti-SARS-CoV2 serum levels.

### Example 23

### Expression of Two Anti-SARS-CoV2 mAb from a Single Plasmid

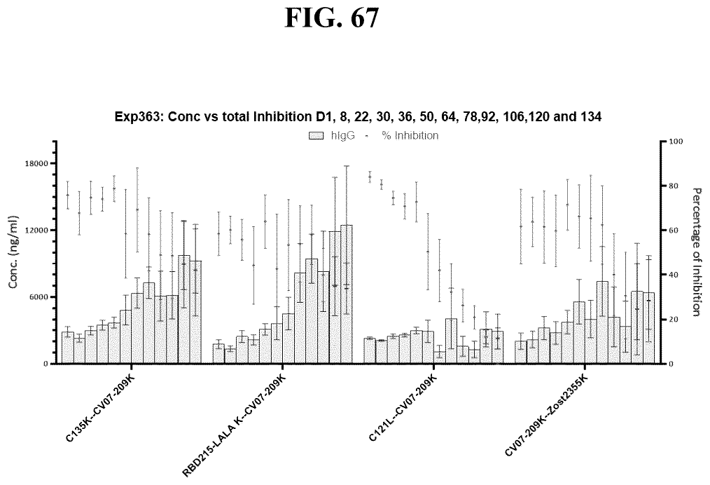

This example describes expression of two SARS-CoV-2 antibodies from a single plasmid (4 different plasmids) in mice produces neutralizing levels of mAb using the following injection protocol. The expressed SARS-CoV-2 antibodies were as follows: first plasmid (C135+CV07-209); second plasmid (RBD215 LALA+CV07-209); third plasmid (C121+CV07-209); and fourth plasmid (CV07-209+Zost-2355) (see Table 7 for sequence information). At day 0, groups of mice were pretreated with 40 mg/kg water-soluble dexamethasone i.p. two hours prior to dosing i.v. with liposomes composing 1100 nmol each of DOTAP/2.5 mol % dexamethasone palmitate/SUV and DMPC/5 mol % dexamethasone palmitate/MLV. After two minutes, mice were dosed i.v. with about 80 ug of a single plasmid DNA containing two expression cassettes for SARS-CoV2-specific mAbs. Mice were bled at days 1, 8, 22, 30, 36, 50, 78, 92, 106, 120, and 134 after treatment and serum mAb protein levels were determined by a human IgG ELISA assay. The results are shown in FIG. 67 (left axis, pink bar graphs represent mean + or −SEM shown in ascending order from day 1 to day 134 for each mAb). The functional bioactivity capacity of SARS-CoV2-specific mAb containing sera to inhibit SARS-CoV2 spike-human ACE2 protein interactions was determined by a commercially-available in vitro SARS-CoV2 spike/ACE2 blocking assay (cPASS, right axis, green dots represent mean + or −SEM shown in ascending order from day 1 to day 134 for each mAb clone, Genscript).

This example demonstrates, as shown in FIG. 67, that this procedure with a single injection of a single expression plasmid results in expression of two SARS-CoV2-specific mAbs from a single plasmid for the course of at least 134 days following this procedure, and that these serum-expressed mAbs sera are functionally capable of blocking SARS-CoV2 spike-human ACE2 interactions for at least 134 days.

### Example 24

### Expression of Two Anti-SARS-CoV2 mAbs by Three Approaches

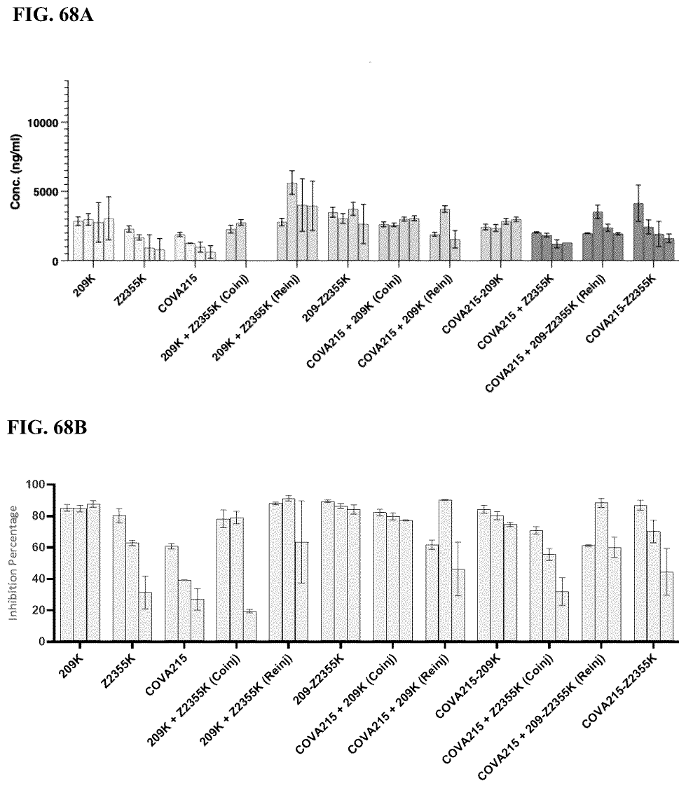

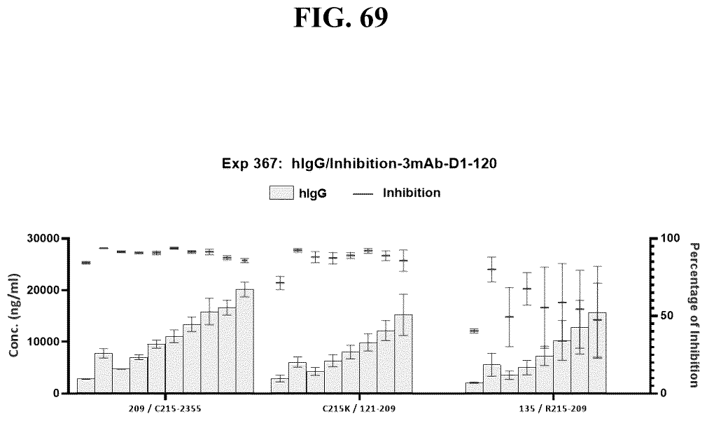

This example describes expression of two anti-SARS-CoV2 mAbs simultaneously by three different approaches: 1) Single injection of a single expression plasmid coding two unique mAbs; 2) Single injection of two unique plasmids simultaneously as a mixture (co-injection); and 3) Two injections of single mAb expression plasmids separated by an amount of time, here 7 days (reinj). The various anti-SARS-CoV2 mAbs expressed are shown in FIGS. 68A-68B (see Table 7 for sequences).

On day 0, groups of mice were pretreated with 40 mg/kg water-soluble dexamethasone i.p. two hours prior to dosing i.v. with liposomes composing 1000 nmol each of DOTAP/2.5 mol % dexamethasone palmitate/SUV and DMPC/5 mol % dexamethasone palmitate/MLV. After two minutes, mice were dosed i.v. with either 75 ug of a single plasmid DNA containing one or two expression cassettes for SARS-CoV2-specific mAbs, or 38 ug each of two plasmids each containing cassettes for one or two mAb clones (co-inject—“coinj”). On day 7, some of these groups of mice underwent an additional injection (re-injection—“reinj”) of dexamethasone retreatment, liposomes dosing, and plasmid DNA as on day 0, and were similarly treated with either 75 ug of a single plasmid DNA containing two expression cassettes for SARS-CoV2-specific mAbs. Mice were bled at day 1, 8, and 15, 22 and serum expression of mAbs was analyzed by a human IgG ELISA assay. Results are shown in FIG. 68A, where each series of bar graphs indicates mean+/−SEM mAb expression or inhibition amount at days 1, 8, 15, and 22 in order from left to right.

FIG. 68B shows the functional capacity of the SARS-CoV2-specific mAb containing sera to inhibit SARS-CoV2 spike-human ACE2 protein interactions determined by a commercially-available in vitro SARS-CoV2 spike/ACE2 blocking assay (cPASS, Genscript). Each series of bar graphs indicates mean+/−SEM mAb expression or inhibition amount at days 1, 8, 15, and 22 in order from left to right.

This examples shows (results in FIGS. 68A-68B) how this protocol produces two anti-SARS-CoV2 mAbs simultaneously by the three approaches tried. All approaches successfully allow for the expression of two mAbs in serum of animals at levels that allow for neutralization of SARS-CoV2/ACE2 interactions.

### Example 25

### Expression of Three Anti-SARS-CoV2 mAbs

This example describes expression of three different anti-SARS-CoV2 mAbs from one or two plasmids based on two weekly injections of the plasmids. This was performed with three different collections of mAbs, as shown in FIG. 69 (sequences in Table 7).

At day 0, groups of mice were pretreated with 40 mg/kg water-soluble dexamethasone i.p. two hours prior to dosing i.v. with liposomes composing 1000 nmol each of DOTAP/2.5 mol % dexamethasone palmitate/SUV and DMPC/5 mol % dexamethasone palmitate/MLV. After two minutes, mice were dosed i.v. with 80 ug of a single plasmid DNA containing one expression cassette for SARS-CoV2-specific mAbs. On day 7, these groups of mice underwent an additional injection of dexamethasone pretreatment, liposomes dosing, and plasmid DNA as on day 0. These groups were treated with 80 ug of a single plasmid DNA containing two expression cassettes for SARS-CoV2-specific mAbs.

Mice were bled at days 1, 8, 15, 21, 36, 50, 64, 78, 92, 106, 120 following their first treatment, and serum expression levels of mAbs were analyzed by a human IgG ELISA assay. The results are shown in FIG. 69, where each series of bar graphs indicates mean+/−SEM mAb expression (left y-axis) at days 1, 8, 15, 21, 36, 50, 64, 78, 92, 106, 120 in order from left to right. In parallel, functional capacities of SARS-CoV2-specific mAb containing sera to inhibit SARS-CoV2 spike-human ACE2 protein interactions were determined by a commercially-available in vitro SARS-CoV2 spike/ACE2 blocking assay across the timecourse (cPASS, Genscript). These inhibition results are shown in FIG. 69 in green. Each series of bar graphs indicates mean+/−SEM mAb inhibition (right y-axis) at days 1, 8, 15, 21, 36, 50, 78, 92, 106, 120.

These examples demonstrate that two weekly injections of one or two DNA expression plasmids encoding a total of three different individual SARS-CoV2-specific mAbs produces fully neutralizing serum levels of three different SARS-CoV2-specific mAbs for the course of at least 70 days following administration, and that these ongoing serum mAbs levels functionally and continuously block SARS-CoV2 spike-human ACE2 for at least 70 days, which is the human equivalent of greater than 10 years. These results indicate that two weekly hedges DNA injections encoding three different SARS-CoV2-specific mAbs produce durable (greater than 10 human years equivalence) fully neutralizing anti-SARS-CoV2 mAb serum levels.

### Example 26

### Expression of Four Anti-SARS-CoV2 mAbs

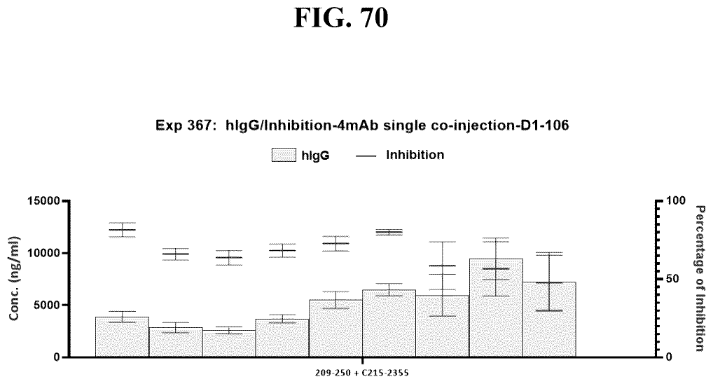

This example describes expression of four (4) anti-SARS-CoV2 mAbs shown in FIG. 70 (see Table 7 for sequence information) using the following protocol. At day 0, groups of mice were pretreated with 40 mg/kg water-soluble dexamethasone i.p. two hours prior to dosing i.v. with liposomes composing 1000 nmol each of DOTAP/2.5 mol % dexamethasone palmitate/SUV and DMPC/5 mol % dexamethasone palmitate/MLV. After two minutes, mice were dosed i.v. with 40 ug each of two plasmids each containing two mAb expression cassettes.

Mice were bled at days 1, 8, 15, 21, 36, 50, 64, 78, 92 and 106 following their first treatment, and serum expression levels of mAbs were analyzed by a human IgG ELISA assay. Results are shown in FIG. 70, where each series of bar graphs indicates mean+/−SEM mAb expression (left y-axis) at days 1, 8, 15, 21, 36, 50, 64, 78, 92, and 106 in order from left to right. In parallel, functional capacities of SARS-CoV2-specific mAb containing sera to inhibit SARS-CoV2 spike-human ACE2 protein interactions were determined by a commercially-available in vitro SARS-CoV2 spike/ACE2 blocking assay across the timecourse (cPASS, Genscript). Each series of bar graphs indicates mean+/−SEM mAb inhibition (right y-axis) at days 1, 8, 15, 21, 36, 50, 78, 92 and 106.

### Example 27

### Expression of Four Anti-SARS-CoV2 mAbs with One Injection

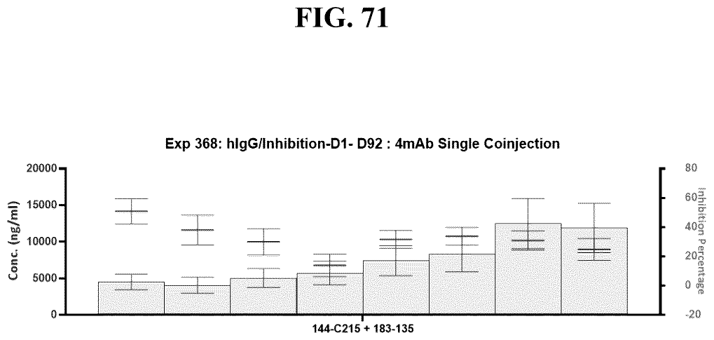

This example describes expression of four anti-SARS-CoV2 mAbs shown in FIG. 71 (see Table 7 for sequence information) using the following protocol. At day 0, groups of mice were pretreated with 40 mg/kg water-soluble dexamethasone i.p. two hours prior to dosing i.v. with liposomes composing 1000 nmol each of DOTAP/2.5 mol % dexamethasone palmitate/SUV and DMPC/5 mol % dexamethasone palmitate/MLV. After two minutes, mice were dosed i.v. with 40 ug each of two plasmids each containing two mAb expression cassettes.

Mice were bled at days 1, 8, 15, 21, 36, 50, 64, 78, and 92 following their first treatment, and serum expression levels of SARS-CoV2 mAbs were analyzed by a human IgG ELISA assay. Results are shown in FIG. 71, where each series of bar graphs indicates mean+/−SEM mAb expression (left y-axis) at days 1, 8, 15, 21, 36, 50, 64, 78, and 92 in order from left to right. In parallel, functional capacities of SARS-CoV2-specific mAb containing sera to inhibit SARS-CoV2 spike-human ACE2 protein interactions were determined by a commercially-available in vitro SARS-CoV2 spike/ACE2 blocking assay across the timecourse (cPASS, Genscript). Each series of bar graphs (in green in FIG. 71) indicates mean+/−SEM mAb inhibition (right y-axis) at days 1, 8, 15, 21, 36, 50, 64, 78 and 92 in order from left to right.

### Example 28

### Expression of Four Anti-SARS-CoV2 mAbs

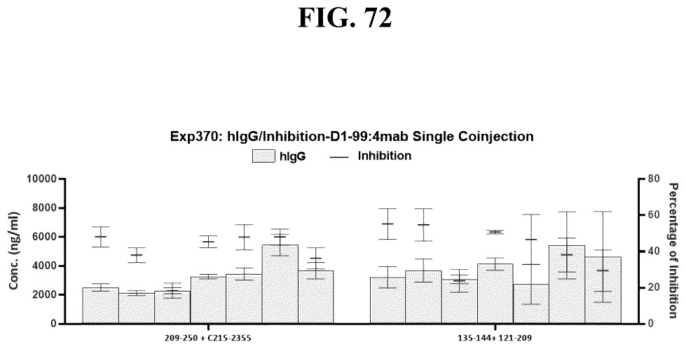

This example describes expression of four anti-SARS-CoV2 mAbs shown in FIG. 72 (see Table 7 for sequence information) using the following protocol. At day 0, groups of mice were pretreated with 40 mg/kg water-soluble dexamethasone i.p. two hours prior to dosing i.v. with liposomes composing 1000 nmol each of DOTAP/2.5 mol % dexamethasone palmitate/SUV and DMPC/5 mol % dexamethasone palmitate/MLV. After two minutes, mice were dosed i.v. with 45 ug each of two plasmids each containing two mAb expression cassettes.

Mice were bled at days 1, 8, 22, 36, 50, 64, 78, 99 following their first treatment, and serum expression levels of mAbs were analyzed by a human IgG ELISA assay. Results are shown in FIG. 72, where each series of bar graphs indicates mean+/−SEM mAb expression (left y-axis) at days 1, 8, 22, 36, 50, 64, 78, 99 in order from left to right. In parallel, functional capacities of SARS-CoV2-specific mAb containing sera to inhibit SARS-CoV2 spike-human ACE2 protein interactions were determined by a commercially-available in vitro SARS-CoV2 spike/ACE2 blocking assay across the timecourse (cPASS, Genscript). These results are shown in green, where each series of bar graphs indicates mean+/−SEM mAb inhibition (right y-axis) at days 1, 8, 22, 36, 50, 64, 78, 99.

These examples demonstrate that a single co-injection of two different single DNA expression plasmids each encoding two different individual SARS-CoV2-specific mAbs (together the co-injection produces a total of four different individual SARS-CoV2-specific mAbs) produces fully neutralizing serum levels of four different SARS-CoV2-specific mAbs for at least 90 days following administration, and that these ongoing serum mAb levels functionally and continuously blocked SARS-CoV2 spike-human ACE2 binding for at least 90 days, which is the human equivalent of greater than 15 years.

### Example 29

### Expression of Four Anti-SARS-CoV2 mAbs with Two Injections

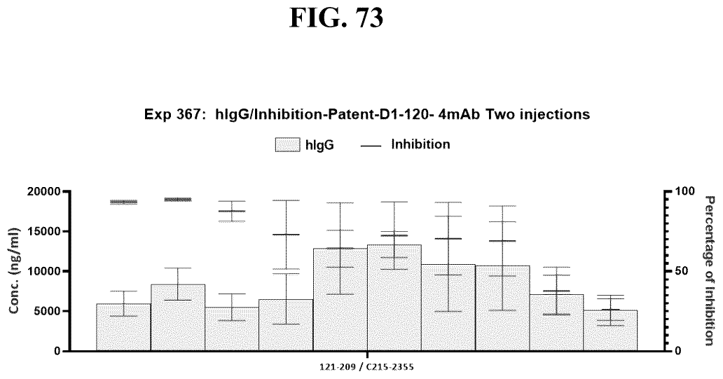

This example describes expression of four anti-SARS-CoV2 mAbs shown in FIG. 73 (see Table 7 for sequence information) using the following protocol. At day 0, groups of mice were pretreated with 40 mg/kg water-soluble dexamethasone i.p. two hours prior to dosing i.v. with liposomes composing 1000 nmol each of DOTAP/2.5 mol % dexamethasone palmitate/SUV and DMPC/5 mol % dexamethasone palmitate/MLV. After two minutes, mice were dosed i.v. with 80 ug of a single plasmid DNA containing two expression cassettes for SARS-CoV2-specific mAbs.

On day 7, mice underwent an additional injection of dexamethasone pretreatment, liposomes dosing, and plasmid DNA as on day 0 (indicated by hashed bar). Mice were treated with 80 ug of a single plasmid DNA containing two expression cassettes for SARS-CoV2-specific mAbs.

Mice were bled at days 1, 8, 15, 21, 36, 50, 64, 78, 92, 106, 120 following their first treatment, and serum expression levels of mAbs were analyzed by a human IgG ELISA assay. Results are shown in FIG. 73, where each series of bar graphs indicates mean+/−SEM mAb expression (left y-axis) at days 1, 8, 15, 21, 36, 50, 64, 78, 92, 106, 120 in order from left to right. In parallel, functional capacities of SARS-CoV2-specific mAb containing sera to inhibit SARS-CoV2 spike-human ACE2 protein interactions were determined by a commercially-available in vitro SARS-CoV2 spike/ACE2 blocking assay across the timecourse (cPASS, Genscript). Each series of bar graphs (in green in FIG. 73) indicates mean+/−SEM mAb inhibition (right y-axis) at days 1, 8, 15, 21, 36, 50, 78, 92, 106, 120.

### Example 30

### Expression of Four Anti-SARS-CoV2 mAbs with Two Injections

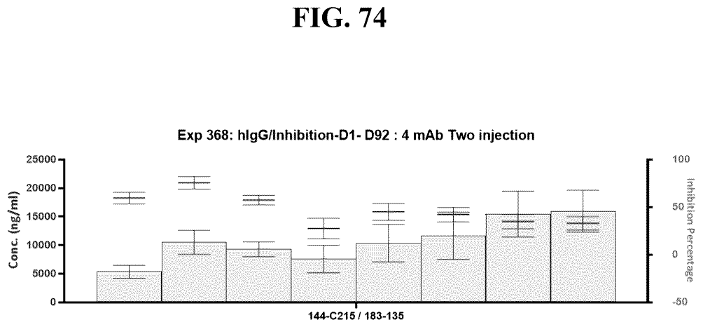

This example describes expression of four anti-SARS-CoV2 mAbs shown in FIG. 74 (see Table 7 for sequence information) using the following protocol. At day 0, groups of mice were pretreated with 40 mg/kg water-soluble dexamethasone i.p. two hours prior to dosing i.v. with liposomes composing 1000 nmol each of DOTAP/2.5 mol % dexamethasone palmitate/SUV and DMPC/5 mol % dexamethasone palmitate/MLV. After two minutes, mice were dosed i.v. with 80 ug of a single plasmid DNA containing two expression cassettes for SARS-CoV2-specific mAbs.

On day 7, some of these groups of mice underwent a second round of injection of dexamethasone pretreatment, liposomes dosing, and plasmid DNA as on day 0 (indicated by dot fill pattern). These groups were treated with 40 ug each of two plasmids each containing two mAb expression cassettes. Mice were bled at days 1, 8, 15, 21, 36, 50, 64, 78, and 92 following their first treatment, and serum expression levels of SARS-CoV2 mAbs were analyzed by a human IgG ELISA assay. Results are shown in FIG. 74, where each series of bar graphs indicates mean+/−SEM mAb expression (left y-axis) at days 1, 8, 15, 21, 36, 50, 64, 78, and 92 in order from left to right. In parallel, functional capacities of SARS-CoV2-specific mAb containing sera to inhibit SARS-CoV2 spike-human ACE2 protein interactions were determined by a commercially-available in vitro SARS-CoV2 spike/ACE2 blocking assay across the timecourse (cPASS, Genscript). Each series of bar graphs (in green) indicates mean+/−SEM mAb inhibition (right y-axis) at days 1, 8, 15, 21, 36, 50, 64, 78 and 92 in order from left to right.

These examples demonstrate that serial, weekly co-injection of two different single DNA expression plasmids each encoding two different individual SARS-CoV2-specific mAbs (together the serial co-injection produces a total of four different individual SARS-CoV2-specific mAbs) produce fully neutralizing serum levels of four different SARS-CoV2-specific mAbs for the course of at least 70 days following administration, and that these ongoing serum mAb levels functionally and continuously blocked SARS-CoV2 spike-human ACE2 binding for at least 70 days, which is the human equivalent of greater than 10 years.

### Example 31

### Expression of Five Anti-SARS-CoV2 mAbs with Two Injections

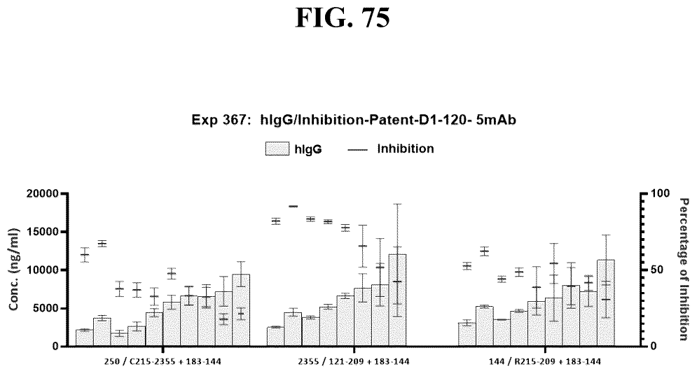

This example describes expression of five anti-SARS-CoV2 mAbs shown in FIG. 75 (see Table 7 for sequence information) using the following protocol. At day 0, groups of mice were pretreated with 40 mg/kg water-soluble dexamethasone i.p. two hours prior to dosing i.v. with liposomes composing 1000 nmol each of DOTAP/2.5 mol % dexamethasone palmitate/SUV and DMPC/5 mol % dexamethasone palmitate/MLV. After two minutes, mice were dosed i.v. with either 80 ug of a single plasmid DNA containing one expression cassette for SARS-CoV2-specific mAbs.

On day 7, these groups of mice underwent an additional injection of dexamethasone pretreatment, liposomes dosing, and plasmid DNA as on day 0. Some groups were treated with 40 ug each of two plasmids each containing two mAb expression cassettes.

Mice were bled at days 1, 8, 15, 21, 36, 50, 64, 78, 92, 106, 120 following their first treatment, and serum expression levels of mAbs were analyzed by a human IgG ELISA assay. Results are shown in FIG. 75, where each series of bar graphs indicates mean+/−SEM mAb expression (left y-axis) at days 1, 8, 15, 21, 36, 50, 64, 78, 92, 106, 120 in order from left to right. In parallel, functional capacities of SARS-CoV2-specific mAb containing sera to inhibit SARS-CoV2 spike-human ACE2 protein interactions were determined by a commercially-available in vitro SARS-CoV2 spike/ACE2 blocking assay across the timecourse (cPASS, Genscript). Each series of bar graphs (shown in green in FIG. 75) indicates mean+/−SEM mAb inhibition (right y-axis) at days 1, 8, 15, 21, 36, 50, 78, 92, 106, 120.

### Example 32

### Expression of Six Anti-SARS-CoV2 mAbs with Single Injection

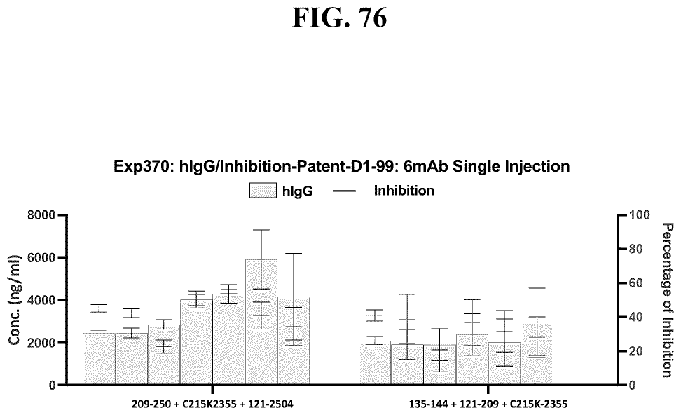

This example describes expression of six anti-SARS-CoV2 mAbs shown in FIG. 76 (see Table 7 for sequence information) using the following protocol. At day 0, groups of mice were pretreated with 40 mg/kg water-soluble dexamethasone i.p. two hours prior to dosing i.v. with liposomes composing 1000 nmol each of DOTAP/2.5 mol % dexamethasone palmitate/SUV and DMPC/5 mol % dexamethasone palmitate/MLV. After two minutes, mice were dosed i.v. with 30 ug each of three plasmids each containing two mAb expression cassettes.

Mice were bled at days 1, 8, 22, 36, 50, 64, 78, 99 following their first treatment, and serum expression levels of mAbs were analyzed by a human IgG ELISA assay. Results are shown in FIG. 76, where each series of bar graphs indicates mean+/−SEM mAb expression (left y-axis) at days 1, 8, 22, 36, 50, 64, 78, 99 in order from left to right. In parallel, functional capacities of SARS-CoV2-specific mAb containing sera to inhibit SARS-CoV2 spike-human ACE2 protein interactions were determined by a commercially-available in vitro SARS-CoV2 spike/ACE2 blocking assay across the time course (cPASS, Genscript). Each series of bar graphs (in green in FIG. 76) indicates mean+/−SEM mAb inhibition (right y-axis) at days 1, 8, 22, 36, 50, 64, 78, 99.

### Example 33

### Expression of Six Anti-SARS-CoV2 mAbs with Two Injections

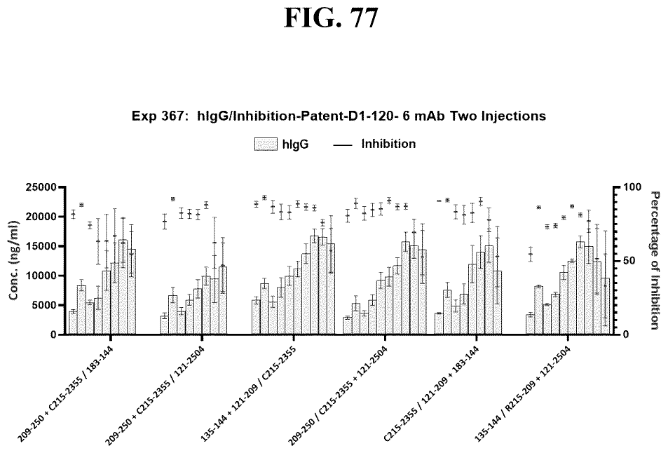

This example describes expression of six anti-SARS-CoV2 mAbs shown in FIG. 77 (see Table 7 for sequence information) using the following protocol. At day 0, groups of mice were pretreated with 40 mg/kg water-soluble dexamethasone i.p. two hours prior to dosing i.v. with liposomes composing 1000 nmol each of DOTAP/2.5 mol % dexamethasone palmitate/SUV and DMPC/5 mol % dexamethasone palmitate/MLV. After two minutes, mice were dosed i.v. with either 80 ug of a single plasmid DNA containing two expression cassettes for SARS-CoV2-specific mAbs, or 40 ug each of two plasmids each containing one or two mAb expression cassettes.

On day 7, these groups of mice underwent an additional injection of dexamethasone pretreatment, liposomes dosing, and plasmid DNA as on day 0. These groups were treated with either 80 ug of a single plasmid DNA containing two expression cassettes for SARS-CoV2-specific mAbs, or 40 ug each of two plasmids each containing two mAb expression cassettes.

Mice were bled at days 1, 8, 15, 21, 36, 50, 64, 78, 92, 106, 120 following their first treatment, and serum expression levels of mAbs were analyzed by a human IgG ELISA assay. Results are shown in FIG. 77, where each series of bar graphs indicates mean+/−SEM mAb expression (left y-axis) at days 1, 8, 15, 21, 36, 50, 64, 78, 92, 106, 120 in order from left to right. In parallel, functional capacities of SARS-CoV2-specific mAb containing sera to inhibit SARS-CoV2 spike-human ACE2 protein interactions were determined by a commercially-available in vitro SARS-CoV2 spike/ACE2 blocking assay across the time course (cPASS, Genscript). Each series of bar graphs (in green in FIG. 77) indicates mean+/−SEM mAb inhibition (right y-axis) at days 1, 8, 15, 21, 36, 50, 78, 92, 106, 120.

### Example 34

### Expression of Six Anti-SARS-CoV2 mAbs 2 or 3 Injections

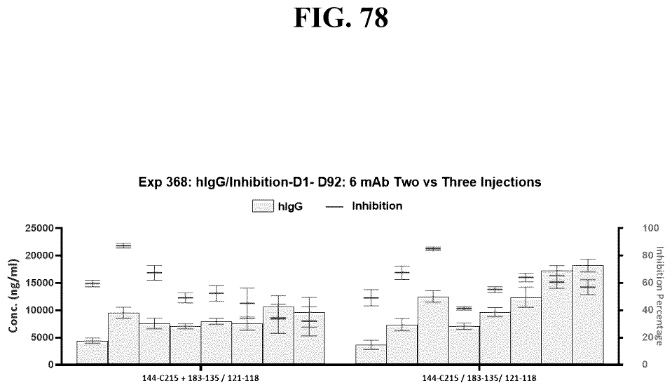

This example describes expression of six anti-SARS-CoV2 mAbs shown in FIG. 78 (see Table 7 for sequence information) using the following protocol. At day 0, groups of mice were pretreated with 40 mg/kg water-soluble dexamethasone i.p. two hours prior to dosing i.v. with liposomes composing 1000 nmol each of DOTAP/2.5 mol % dexamethasone palmitate/SUV and DMPC/5 mol % dexamethasone palmitate/MLV. After two minutes, mice were dosed i.v. with either 80 ug of a single plasmid DNA containing two expression cassettes for SARS-CoV2-specific mAb, or 40 ug each of two plasmids each containing one or two mAb expression cassettes.

On day 7, these groups of mice underwent a second round of injection of dexamethasone pretreatment, liposomes dosing, and plasmid DNA as on day 0 (indicated by dot fill pattern). These groups were treated 40 ug each of two plasmids each containing two mAb expression cassettes.

On day 14, some of these groups of mice underwent a third round of injection of dexamethasone pretreatment, liposomes dosing, and plasmid DNA as on day 0. These groups were treated with 80 ug of a single plasmid DNA containing two expression cassettes for SARS-CoV2-specific mAb.

Mice were bled at days 1, 8, 15, 21, 36, 50, 64, 78, and 92 following their first treatment, and serum expression levels of SARS-CoV2 mAbs were analyzed by a human IgG ELISA assay. Results are shown in FIG. 78, where each series of bar graphs indicates mean+/−SEM mAb expression (left y-axis) at days 1, 8, 15, 21, 36, 50, 64, 78, and 92 in order from left to right. In parallel, functional capacities of SARS-CoV2-specific mAb containing sera to inhibit SARS-CoV2 spike-human ACE2 protein interactions were determined by a commercially-available in vitro SARS-CoV2 spike/ACE2 blocking assay across the timecourse (cPASS, Genscript). Each series of bar graphs (in green in FIG. 78) indicates mean+/−SEM mAb inhibition (right y-axis) at days 1, 8, 15, 21, 36, 50, 64, 78 and 92 in order from left to right.

These examples demonstrate that serial co-injection of three different single DNA expression plasmids each encoding two different individual SARS-CoV2-specific mAbs (together the serial injections produce a total of six different individual SARS-CoV2-specific mAbs) each produce fully neutralizing serum levels of six different SARS-CoV2-specific mAbs for the course of at least 90 days following administration, and that these ongoing serum mAbs levels produced functionally block SARS-CoV2 spike-human ACE2 binding and that these functionally and continuously blocked SARS-CoV2 spike-human ACE2 binding for at least 90 days, which is the human equivalent of greater than 15 years.

### Example 35

### Expression of Eight Anti-SARS-CoV2 mAbs with Two Injections

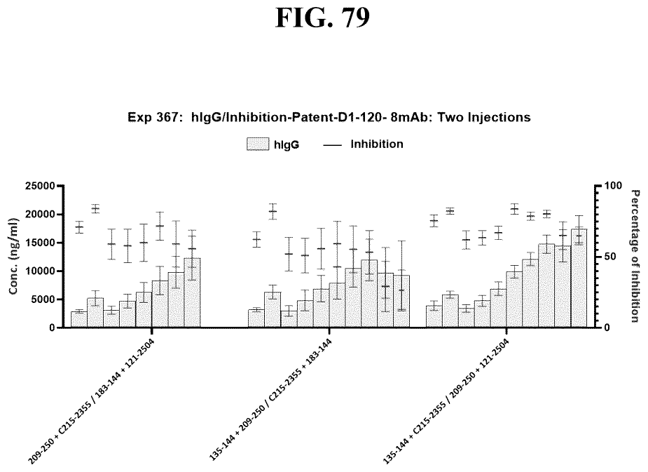

This example describes expression of eight anti-SARS-CoV2 mAbs shown in FIG. 79 (see Table 7 for sequence information) using the following protocol. At day 0, groups of mice were pretreated with 40 mg/kg water-soluble dexamethasone i.p. two hours prior to dosing i.v. with liposomes composing 1000 nmol each of DOTAP/2.5 mol % dexamethasone palmitate/SUV and DMPC/5 mol % dexamethasone palmitate/MLV. After two minutes, mice were dosed i.v. with 40 ug each of two plasmids each containing two mAb expression cassettes.

On day 7, mice underwent an additional injection of dexamethasone pretreatment, liposome dosing, and plasmid DNA as on day 0. Mice were treated with 40 ug each of two plasmids each containing two mAb expression cassettes.

Mice were bled at days 1, 8, 15, 21, 36, 50, 64, 78, 92, 106, 120 following their first treatment, and serum expression levels of mAbs were analyzed by a human IgG ELISA assay. Results are shown in FIG. 79, where each series of bar graphs indicates mean+/−SEM mAb expression (left y-axis) at days 1, 8, 15, 21, 36, 50, 64, 78, 92, 106, 120 in order from left to right. In parallel, functional capacities of SARS-CoV2-specific mAb containing sera to inhibit SARS-CoV2 spike-human ACE2 protein interactions were determined by a commercially-available in vitro SARS-CoV2 spike/ACE2 blocking assay across the time course (cPASS, Genscript). Each series of bar graphs (green in FIG. 79) indicates mean+/−SEM mAb inhibition (right y-axis) at days 1, 8, 15, 21, 36, 50, 78, 92, 106, 120.

### Example 36

### Expression of Eight Anti-SARS-CoV2 mAbs with Two Injections

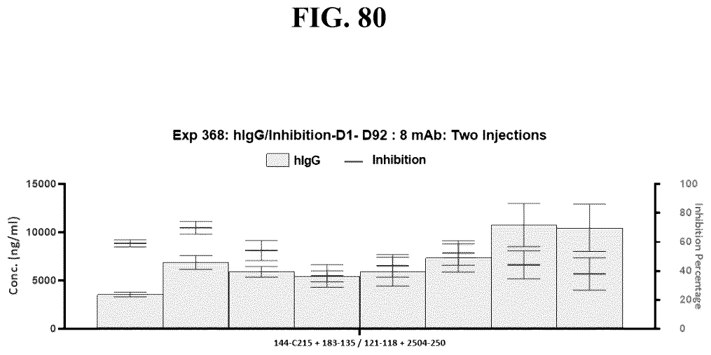

This example describes expression of eight anti-SARS-CoV2 mAbs shown in FIG. 80 (see Table 7 for sequence information) using the following protocol. At day 0, groups of mice were pretreated with 40 mg/kg water-soluble dexamethasone i.p. two hours prior to dosing i.v. with liposomes composing 1000 nmol each of DOTAP/2.5 mol % dexamethasone palmitate/SUV and DMPC/5 mol % dexamethasone palmitate/MLV. After two minutes, mice were dosed i.v. with 40 ug each of two plasmids each containing two mAb expression cassettes.

On day 7, mice underwent a second round of injection of dexamethasone pretreatment, liposomes dosing, and plasmid DNA as on day 0. These mice were treated with 40 ug each of two plasmids each containing two mAb expression cassettes.

Mice were bled at days 1, 8, 15, 21, 36, 50, 64, 78, and 92 following their first treatment, and serum expression levels of SARS-CoV2 mAbs were analyzed by a human IgG ELISA assay. Results are shown in FIG. 80, where each series of bar graphs indicates mean+/−SEM mAb expression (left y-axis) at days 1, 8, 15, 21, 36, 50, 64, 78, and 92 in order from left to right. In parallel, functional capacities of SARS-CoV2-specific mAb containing sera to inhibit SARS-CoV2 spike-human ACE2 protein interactions were determined by a commercially-available in vitro SARS-CoV2 spike/ACE2 blocking assay across the time course (cPASS, Genscript). Each series of bar graphs (shown in green in FIG. 80) indicates mean+/−SEM mAb inhibition (right y-axis) at days 1, 8, 15, 21, 36, 50, 64, 78 and 92 in order from left to right.

### Example 37

### Expression of Eight Anti-SARS-CoV2 mAbs with Three Injections

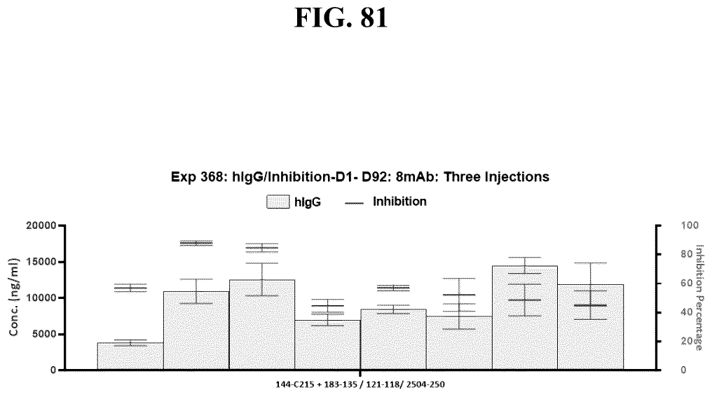

This example describes expression of eight anti-SARS-CoV2 mAbs shown in FIG. 81 (see Table 7 for sequence information) using the following protocol. At day 0, groups of mice were pretreated with 40 mg/kg water-soluble dexamethasone i.p. two hours prior to dosing i.v. with liposomes composing 1000 nmol each of DOTAP/2.5 mol % dexamethasone palmitate/SUV and DMPC/5 mol % dexamethasone palmitate/MLV. After two minutes, mice were dosed i.v. with 40 ug each of two plasmids each containing two mAb expression cassettes.

On day 7, mice underwent a second round of injection of dexamethasone pretreatment, liposomes dosing, and plasmid DNA as on day 0. These groups were treated with 80 ug of a single plasmid DNA containing two expression cassettes for SARS-CoV2-specific mAbs.

On day 14, mice underwent a third round of injection of dexamethasone pretreatment, liposomes dosing, and plasmid DNA as on day 0 (indicated by hashed bar). These groups were treated with 80 ug each of a single plasmid containing two mAb expression cassettes.

Mice were bled at days 1, 8, 15, 21, 36, 50, 64, 78, and 92 following their first treatment, and serum expression levels of SARS-CoV2 mAbs were analyzed by a human IgG ELISA assay. Results are shown in FIG. 81, where each series of bar graphs indicates mean+/−SEM mAb expression (left y-axis) at days 1, 8, 15, 21, 36, 50, 64, 78, and 92 in order from left to right. In parallel, functional capacities of SARS-CoV2-specific mAb containing sera to inhibit SARS-CoV2 spike-human ACE2 protein interactions were determined by a commercially-available in vitro SARS-CoV2 spike/ACE2 blocking assay across the time course (cPASS, Genscript). Each series of bar graphs (green in FIG. 81) indicates mean+/−SEM mAb inhibition (right y-axis) at days 1, 8, 15, 21, 36, 50, 64, 78 and 92 in order from left to right.

### Example 38

### Expression of Eight Anti-SARS-CoV2 mAbs

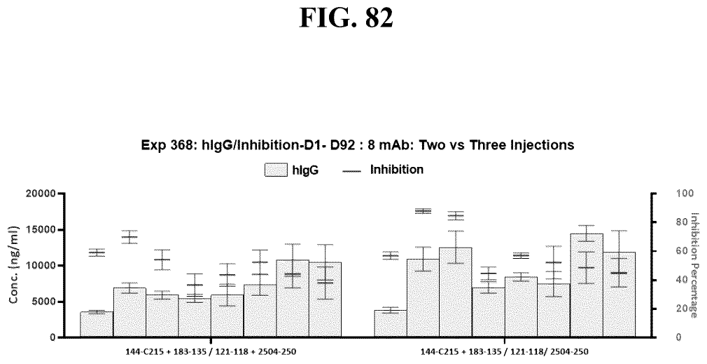

This example describes expression of eight anti-SARS-CoV2 mAbs shown in FIG. 82 (see Table 7 for sequence information) using the following protocol. At day 0, groups of mice were pretreated with 40 mg/kg water-soluble dexamethasone i.p. two hours prior to dosing i.v. with liposomes composing 1000 nmol each of DOTAP/2.5 mol % dexamethasone palmitate/SUV and DMPC/5 mol % dexamethasone palmitate/MLV. After two minutes, mice were dosed i.v. with 40 ug each of two plasmids each containing one or two mAb expression cassettes.

On day 7, these groups of mice underwent a second round of injection of dexamethasone pretreatment, liposomes dosing, and plasmid DNA as on day 0. These groups were treated with either 80 ug of a single plasmid DNA containing two expression cassettes for SARS-CoV2-specific mAb, or 40 ug each of two plasmids each containing two mAb expression cassettes.

On day 14, some of these groups of mice underwent a third round of injection of dexamethasone pretreatment, liposomes dosing, and plasmid DNA as on day 0 (indicated by hashed bar). These groups were treated with 80 ug of a single plasmid DNA containing two expression cassettes for SARS-CoV2-specific mAbs.

Mice were bled at days 1, 8, 15, 21, 36, 50, 64, 78, and 92 following their first treatment, and serum expression levels of SARS-CoV2 mAbs were analyzed by a human IgG ELISA assay. Results are shown in FIG. 82, where each series of bar graphs indicates mean+/−SEM mAb expression (left y-axis) at days 1, 8, 15, 21, 36, 50, 64, 78, and 92 in order from left to right. In parallel, functional capacities of SARS-CoV2-specific mAb containing sera to inhibit SARS-CoV2 spike-human ACE2 protein interactions were determined by a commercially-available in vitro SARS-CoV2 spike/ACE2 blocking assay across the time course (cPASS, Genscript). Each series of bar graphs (green in FIG. 82) indicates mean+/−SEM mAb inhibition (right y-axis) at days 1, 8, 15, 21, 36, 50, 64, 78 and 92 in order from left to right.

### Example 39

### Expression of 10 Anti-SARS-CoV2 mAbs with Other Protein and mAbs

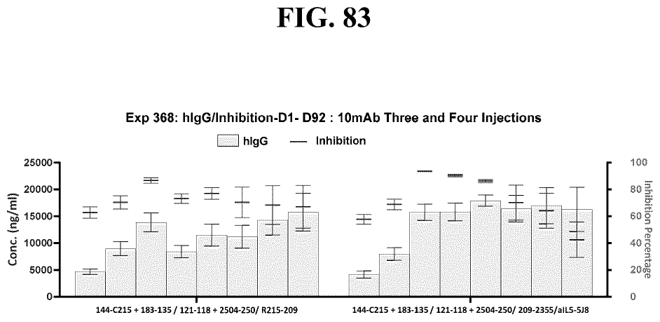

This example describes expression of ten anti-SARS-CoV2 mAbs shown in FIG. 83 (see Table 7 for sequence information), and other proteins and mAbs, using the following protocol. At day 0, groups of mice were pretreated with 40 mg/kg water-soluble dexamethasone i.p. two hours prior to dosing i.v. with liposomes composing 1000 nmol each of DOTAP/2.5 mol % dexamethasone palmitate/SUV and DMPC/5 mol % dexamethasone palmitate/MLV. After two minutes, mice were dosed i.v. with 40 ug each of two plasmids each containing one or two mAb expression cassettes.

On day 7, these groups of mice underwent a second round of injection of dexamethasone pretreatment, liposomes dosing, and plasmid DNA as on day 0 (indicated by dot fill pattern). These groups were treated with 40 ug each of two plasmids each containing two mAb expression cassettes.

On day 14, these groups of mice underwent a third round of injection of dexamethasone pretreatment, liposomes dosing, and plasmid DNA as on day 0. These groups were treated with 80 ug of a single plasmid DNA containing two expression cassettes for SARS-CoV2-specific mAb clones.

On day 21, some of these groups of mice underwent a fourth round of injection of dexamethasone pretreatment, liposomes dosing, and plasmid DNA as on day 0. These groups were treated with 80 ug of a single plasmid DNA containing two expression cassettes for non-SARS-CoV2-related proteins. These non-SARS-CoV2-related proteins included mepoluzimab (aIL5), and anti-influenza A hemagglutinin H1 (5J8).

Mice were bled at days 1, 8, 15, 21, 36, 50, 64, 78, and 92 following their first treatment, and serum expression levels of SARS-CoV2 mAbs were analyzed by a human IgG ELISA assay. Results are shown in FIG. 83, where each series of bar graphs indicates mean+/−SEM mAb expression (left y-axis) at days 1, 8, 15, 21, 36, 50, 64, 78, and 92 in order from left to right. In parallel, functional capacities of SARS-CoV2-specific mAb containing sera to inhibit SARS-CoV2 spike-human ACE2 protein interactions were determined by a commercially-available in vitro SARS-CoV2 spike/ACE2 blocking assay across the time course (cPASS, Genscript). Each series of bar graphs (green in FIG. 83) indicates mean+/−SEM mAb inhibition (right y-axis) at days 1, 8, 15, 21, 36, 50, 64, 78 and 92 in order from left to right.

### Example 40

### Expression of 11 Anti-SARS-CoV2 mAbs with Other Protein and mAbs

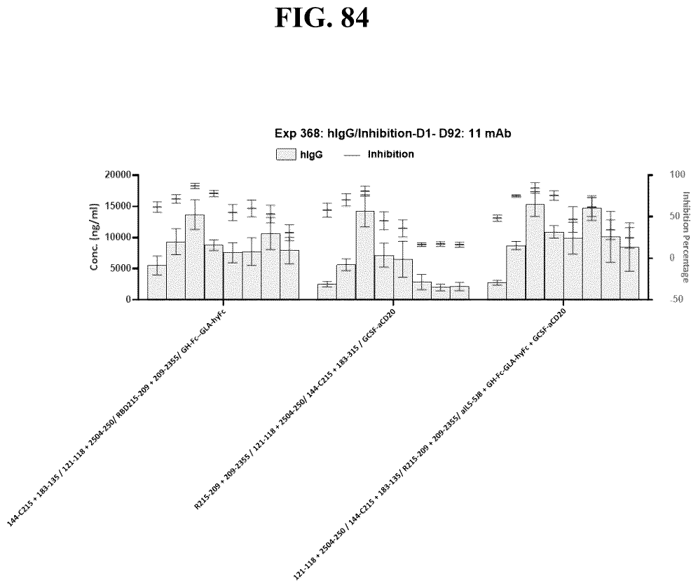

This example describes expression of eleven anti-SARS-CoV2 mAbs shown in FIG. 84 (see Table 7 for sequence information), and other proteins and mAbs, using the following protocol. At day 0, groups of mice were pretreated with 40 mg/kg water-soluble dexamethasone i.p. two hours prior to dosing i.v. with liposomes composing 1000 nmol each of DOTAP/2.5 mol % dexamethasone palmitate/SUV and DMPC/5 mol % dexamethasone palmitate/MLV. After two minutes, mice were dosed i.v. with 40 ug each of two plasmids each containing two mAb expression cassettes.

On day 7, these groups of mice underwent a second round of injection of dexamethasone pretreatment, liposomes dosing, and plasmid DNA as on day 0. These groups were treated with 40 ug each of two plasmids each containing two mAb expression cassettes.

On day 14, these groups of mice underwent a third round of injection of dexamethasone pretreatment, liposomes dosing, and plasmid DNA as on day 0. These groups were treated with 40 ug each of two plasmids each containing two mAb expression cassettes.

On day 21, these groups of mice underwent a fourth round of injection of dexamethasone pretreatment, liposomes dosing, and plasmid DNA as on day 0. These groups were treated with either 80 ug of a single plasmid DNA containing two or more expression cassettes for non-SARS-CoV2-related proteins, 40 ug each of two plasmids each containing two non-SARS-CoV2-related proteins, or 25 ug each of three plasmids each containing two non-SARS-CoV2-specific protein expression cassettes. These non-SARS-CoV2-related proteins included human growth hormone (GH), galactosidase alpha (GLA), G-CSF, and mAbs rituximab (aCD20), mepoluzimab (aIL5), and anti-influenza A hemagglutinin H1 (5J8).

Mice were bled at days 1, 8, 15, 21, 36, 50, 64, 78, and 92 following their first treatment, and serum expression levels of SARS-CoV2 mAbs were analyzed by a human IgG ELISA assay. Results are shown in FIG. 84, where each series of bar graphs indicates mean+/−SEM mAb expression (left y-axis) at days 1, 8, 15, 21, 36, 50, 64, 78, and 92 in order from left to right. In parallel, functional capacities of SARS-CoV2-specific mAb containing sera to inhibit SARS-CoV2 spike-human ACE2 protein interactions were determined by a commercially-available in vitro SARS-CoV2 spike/ACE2 blocking assay across the time course (cPASS, Genscript). Each series of bar graphs (green in FIG. 84) indicates mean+/−SEM mAb inhibition (right y-axis) at days 1, 8, 15, 21, 36, 50, 64, 78 and 92 in order from left to right.

These examples demonstrate that serial co-injection of up to six different single DNA expression plasmids, each plasmid encoding two different individual SARS-CoV2-specific mAbs (together the serial injections produce a total of up to 11 different individual SARS-CoV2-specific mAbs) produce neutralizing serum levels of up to 11 different SARS-CoV2-specific mAbs for the course of at least 90 days following administration, and that these ongoing serum mAbs levels functionally and continuously blocked SARS-CoV2 spike-human ACE2 binding for at least 90 days, which is the human equivalent of greater than 15 years.

### Example 41

### Expression of 10 Anti-SARS-CoV2 mAbs with Other Protein and mAbs

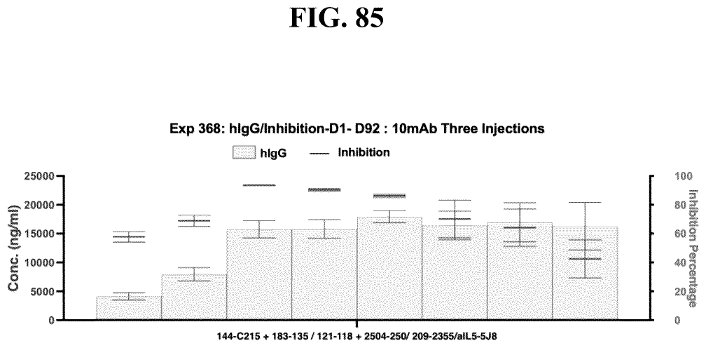

This example describes expression of ten anti-SARS-CoV-2 mAbs shown in FIG. 85 (see Table 7 for sequence information), and other non-Sars-CoV-2 mAbs, using the following protocol. At day 0, groups of mice were pretreated with 40 mg/kg water-soluble dexamethasone i.p. two hours prior to dosing i.v. with liposomes composing 1000 nmol each of DOTAP/2.5 mol % dexamethasone palmitate/SUV and DMPC/5 mol % dexamethasone palmitate/MLV. After two minutes, mice were dosed i.v. with 40 ug each of two plasmids each containing two mAb expression cassettes.

On day 7, mice underwent a second round of injection of dexamethasone pretreatment, liposomes dosing, and plasmid DNA as on day 0 (indicated by dot fill pattern). Mice were treated with 40 ug each of two plasmids each containing two mAb expression cassettes.

On day 14, mice underwent a third round of injection of dexamethasone pretreatment, liposomes dosing, and plasmid DNA as on day 0 (indicated by hashed bar). These mice were treated with 80 ug of a single plasmid DNA containing two expression cassettes for SARS-CoV2-specific mAbs.

On day 21, mice underwent a fourth round of injection of dexamethasone pretreatment, liposomes dosing, and plasmid DNA as on day 0. These groups were treated with 80 ug of a single plasmid DNA containing two expression cassettes for non-SARS-CoV2-related proteins. These non-SARS-CoV2-related proteins included mepoluzimab biosimilar (aTL5), and anti-influenza A hemagglutinin H1 (5J8).

Mice were bled at days 1, 8, 15, 21, 36, 50, 64, 78, and 92 following their first treatment, and serum expression levels of SARS-CoV2 mAbs were analyzed by a human IgG ELISA assay. Results are shown in FIG. 85, where each series of bar graphs indicates mean+/−SEM mAb expression (left y-axis) at days 1, 8, 15, 21, 36, 50, 64, 78, and 92 in order from left to right. In parallel, functional capacities of SARS-CoV2-specific mAb containing sera to inhibit SARS-CoV2 spike-human ACE2 protein interactions were determined by a commercially-available in vitro SARS-CoV2 spike/ACE2 blocking assay across the time course (cPASS, Genscript). Each series of bar graphs indicates (green in FIG. 85) mean+/−SEM mAb inhibition (right y-axis) at days 1, 8, 15, 21, 36, 50, 64, 78 and 92 in order from left to right.

This example demonstrates that serial co-injection of a total of 6 different single DNA expression plasmids, 5 of which encode two different individual SARS-CoV2-specific mAbs and the sixth encodes the heavy and light chains cDNAs of mAb 5J8, which is directed against the 1918 pandemic influenza virus. Together these serial injections produced neutralizing levels of a total of 10 different individual SARS-CoV2-specific serum mAb proteins together with one 1918 pandemic influenza specific serum mAb protein. These injections produced neutralizing serum levels of all 10 different SARS-CoV2-specific mAbs as well as neutralizing serum levels of the 1918 pandemic influenza-specific mAbs for the course of at least 90 days following administration, and that these ongoing SARS-CoV2-specific mAbs serum levels functionally and continuously blocked SARS-CoV2 spike-human ACE2 binding. In addition, hedges produced anti-pandemic influenza A mAb 5J8 serum levels neutralized the Cal/09 pandemic influenza virus strain for at least 90 days, which is the human equivalent of greater than 15 years. This means that a total of four serial DNA vector administrations can neutralize both the SARS-CoV-2 virus as well as a pandemic influenza virus for decades thereafter.

### Example 42

### SARS-CoV2 Inhibition by 14 Hours Post-Treatment

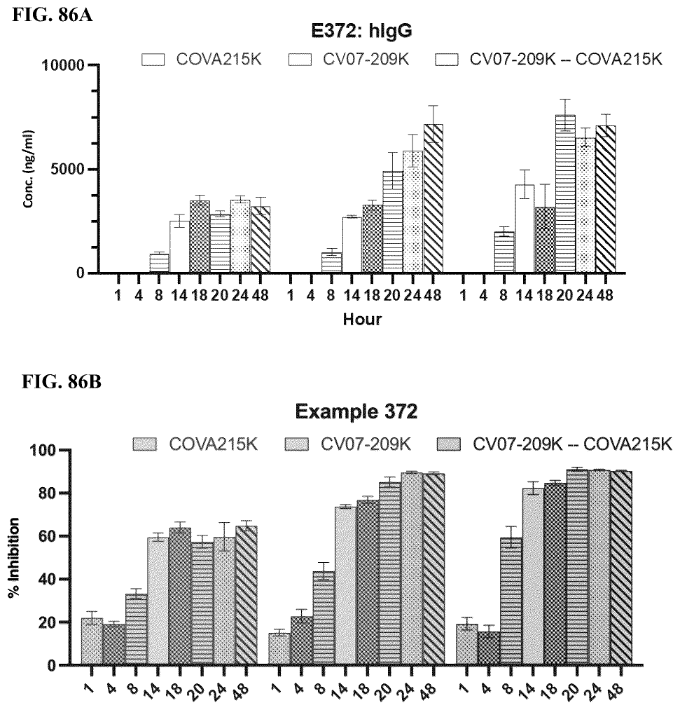

This example describes inhibition of SARS-CoV2 by 14 hours post-treatment with the anti-SARS-CoV-2 mAbs shown in FIGS. 86A-86B (see Table 7 for sequence information). At day 0, groups of mice were pretreated with 40 mg/kg water-soluble dexamethasone i.p. two hours prior to dosing i.v. with liposomes composing 1000 nmol each of DOTAP/2.5 mol % dexamethasone palmitate/SUV and DMPC/5 mol % dexamethasone palmitate/MLV. After two minutes, mice were dosed i.v. with 80 ug of a single plasmid DNA containing one or two expression cassettes for SARS-CoV2-specific mAbs clones.

Mice were bled at 1, 4, 8, 14, 18, 20, 24, and 48 hours following treatment with plasmid DNA, and serum expression levels of mAbs were analyzed by a human IgG ELISA assay. Results are shown in FIG. 86A, where each series of bar graphs indicates mean+/−SEM mAb expression at the indicated times (hr). In parallel, functional capacities of SARS-CoV2-specific mAb containing sera to inhibit SARS-CoV2 spike-human ACE2 protein interactions were determined by a commercially-available in vitro SARS-CoV2 spike/ACE2 blocking assay across the time course (cPASS, Genscript). Results are shown in FIG. 86B, where each series of bar graphs indicates mean+/−SEM mAb inhibition at the indicted time in hours following treatment. This example uses assaying a time course of the ability of anti-SARS-CoV-2 mAb serum levels produced over time between one and 24 hours after a single anti-SARS-CoV-2 DNA vector administration encoding either one or two anti-SARS-CoV-2 mAb heavy and light chain cDNAs to functionally block SARS-CoV2 spike-human ACE2 binding. The results demonstrate that SARS-CoV2 spike-human ACE2 binding is efficiently blocked within 8 hours of one IV hedges DNA vector injection encoding either one or two anti-SARS-CoV-2 mAbs. In contrast, neutralizing protection following two different anti-SARS-CoV-2 vaccine administration generally requires five weeks.

### Example 43

### Simultaneous Expression of Multiple Different mAb and Genes

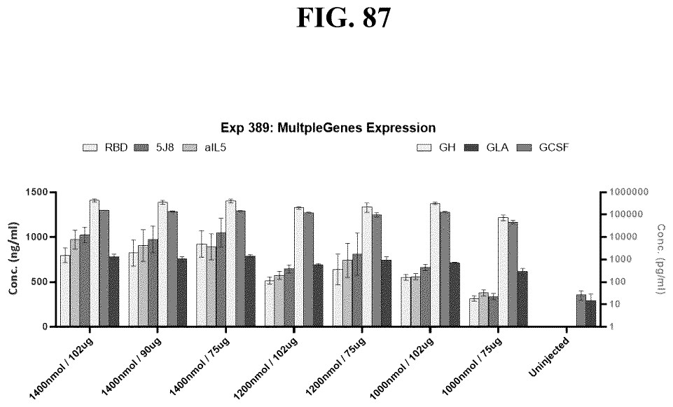

This example describes the simultaneous expression of six different mAb and genes using a single injection. Four mice per group were injected IP with 40 mg/kg water-soluble dexamethasone. Two hours later, mice were injected i.v. with cationic liposomes containing 2.5% dexamethasone 21-palmitate, at doses shown in FIG. 87, as well as 1000 nmol DMPC liposomes containing 5% dex palmitate. Two minutes following the first i.v. injection, mice were injected with 25 ug each, 30 ug each, or 34 ug each of three DNA plasmids: one encoding anti-IL5 and 5J8, one encoding hGH and hGCSF, and one encoding an anti-SARS-CoV2 and GLA. Mice were bled the following day and sera analyzed for expression of target genes. Expression results are shown in FIG. 87. This example demonstrates that a single co-injection of three different DNA vectors, each vector encoding either two or three different human genes, produces significant serum levels of all six different human proteins.

### Example 44

### Controlled Gene Expression with Various Eukaryotic Promoters

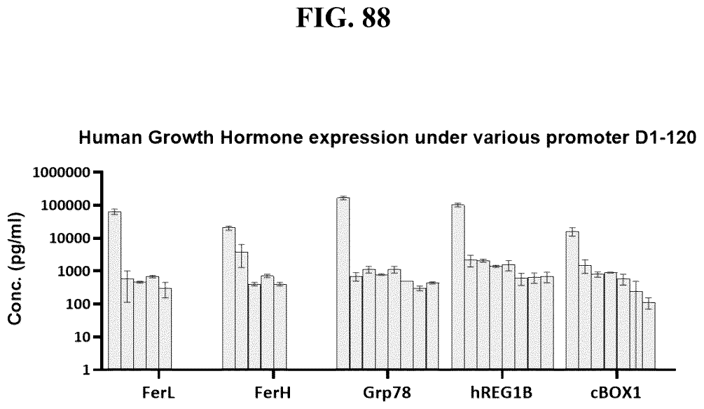

This example describes the use of various eukaryotic promoters to express a target gene (human growth hormone). At day 0, groups of mice were pretreated with 40 mg/kg water-soluble dexamethasone i.p. two hours prior to dosing i.v. with liposomes composing 1100 nmol ea of DOTAP/2.5 mol % dexamethasone palmitate/SUV and DMPC/5 mol % dexamethasone palmitate/MLV. After two minutes, mice were dosed i.v. with 75 ug of various single plasmid DNA construct each containing an expression cassette for human growth hormone-Fc fusion driven by the promoters of heterologous genes, shown in FIG. 88. Mice were bled at days 1, 8, 22, 29, 43, 50, 84 and 120 after treatment and serum mAb protein levels were determined by a human IgG ELISA assay. Bar graphs shown for each promoter in FIG. 88 are in ascending order from day 1 to day 120 for each. Mean hGH-FC expression and SEM are displayed.

This data shows that selected changes in the identity and composition of the DNA vector promoter element within the DNA vector expression cassette allows for longitudinal control over the magnitude of protein expression and bioactivity without the use of gene switches or any other additional modification.

### Example 45

### Testing of 11 Different hGLA DNA Vectors

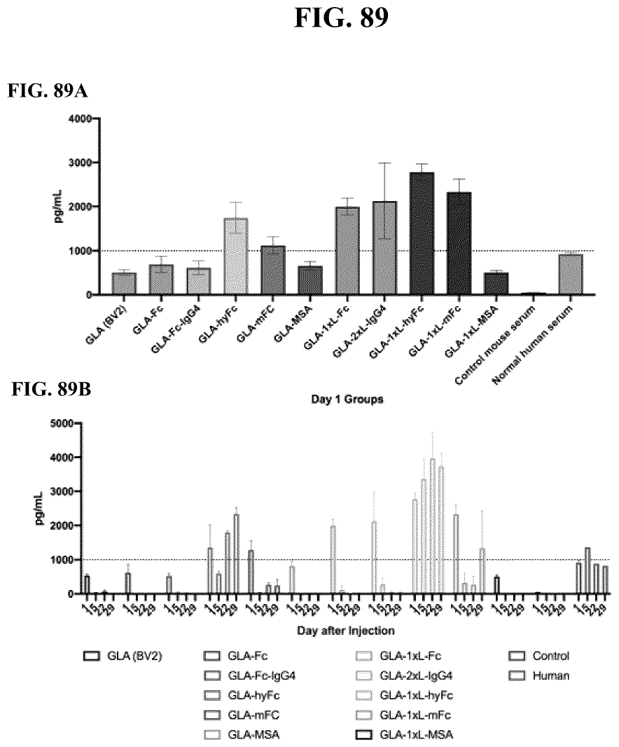

This example describes simultaneously testing 11 different hGLA DNA vectors, showing that they produce a spectrum of serum levels over time. This allowed, for example to identify vectors that maintain hGLA levels in the 1-19 ng/ml range. On day 0, groups of mice were pretreated with 40 mg/kg water-soluble dexamethasone IP two hours prior to i.v. injection. Liposome injection i.v. contained 1000 nmol each of DOTAP SUV with 2.5 mol % dexamethasone 21-palmitate and DMPC MLV with 5 mol % dexamethasone palmitate/MLV. Two minutes later, 75 ug DNA was injected i.v., with constructs encoding GLA as shown in FIGS. 89A-89B. Mice were bled the following day and every 7 or 14 days thereafter and sera assessed for hGLA protein production. Results are shown in FIGS. 89A-89B.

### Example 46

### Fc-Modified Protein Expression

FIG. 89A shows that multiple different FC modified human GLA cDNA-encoded hedges DNA vectors produce therapeutic serum hGLA levels (>1 ng/ml) at day one after administration. However by day eight (FIG. 89B), only the HyFc, and particularly the Hy-Fc 1xL-containing the hGLA DNA vectors remain within the GLA therapeutic range. All other 9, Fc modified DNA vectors have dropped below therapeutic levels by day eight. These results show that optimizing the Fc portion of Fc hybrid DNA vectors can greatly improve serum half-life of modified Fc containing DNA vectors.

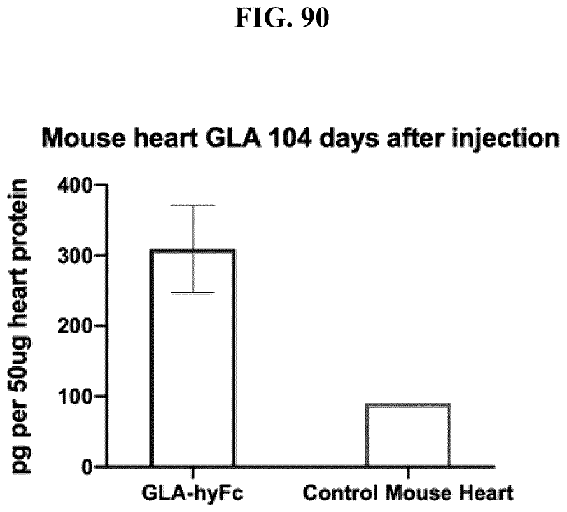

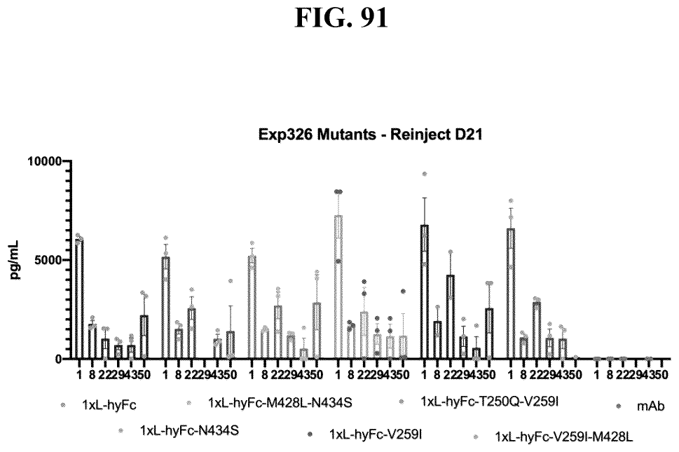

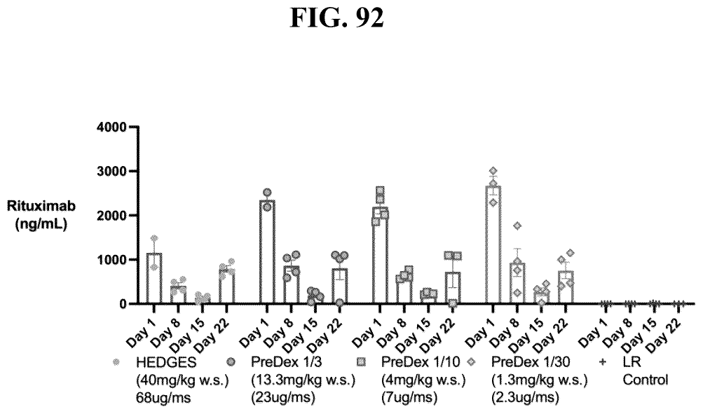

FIG. 90 demonstrates that this Fc modifications is of clinical importance, as the use of this hyFc hGLA containing DNA vector significantly increases hGLA tissue levels in heart 104 days after a single hedges DNA vector administration. Heart is one of the most damaged target organs in GLA deficient Fabry's patients. For this example that Fc-modified GLA can be expressed in heart tissue at therapeutic levels 104 days after injection of vector: on day 0, groups of mice were pretreated with 40 mg/kg water-soluble dexamethasone IP two hours prior to i.v. injection. Liposome injection i.v. contained 1000 nmol each of DOTAP SUV with 2.5 mol % dexamethasone 21-palmitate and DMPC MLV with 5 mol % dexamethasone 21-palmitate. Two minutes later, 75 ug DNA was injected i.v., with constructs encoding GLA-Fc with point mutations as shown in FIG. 91. Mice were sacrificed at day 104 after injection. Hearts were perfused with PBS and then removed to lysis buffer on ice. Hearts were sonicated and protein quantified by Lowry. 50 ug total protein was loaded into wells and GLA measured by ELISA. Heart tissue expression levels are shown in FIG. 92.

### Example 47

### Various Fc Protein Mutations Affect Expression

This example compares the expression of various mutated Fc regions (shown in FIG. 91) for GLA-Fc expression. On day 0, groups of mice were pretreated with 40 mg/kg water-soluble dexamethasone IP two hours prior to i.v. injection. Liposome injection i.v. contained 1000 nmol each of DOTAP SUV with 2.5 mol % dexamethasone 21-palmitate and DMPC MLV with 5 mol % dexamethasone 21-palmitate. Two minutes later, 75 ug DNA was injected i.v., with constructs encoding GLA with point mutations as shown in FIG. 91. Mice were bled the following day and every 7 or 14 days thereafter and sera assessed for hGLA protein production. FIG. 91 demonstrates that targeted single or several DNA base modification of the HyFC-ixL-hGLA DNA vector via site directed mutagenesis allows precisely targeted single base modification of hybrid Fc DNA vector encoded protein function.

### Example 48

### Use of Low Dose of Dexamethasone

This example describes the use of low dose dexamethasone pretreatment does not interfere with the durability of protein expression (and acute expression may be augmented). On day 0, groups of 25 gram mice were pretreated with the indicated amounts (in FIG. 92) of water-soluble dexamethasone IP two hours prior to i.v. injection. Liposome injection i.v. contained 1000 nmol each of DOTAP SUV with 2.5 mol % dexamethasone 21-palmitate and DMPC MLV with 5 mol % dexamethasone 21-palmitate. Two minutes later, 75 ug of rituximab-biosimilar expression DNA plasmid was injected i.v. Mice were bled the following day and at day 8, 15, and 22. Serum expression of rituximab-biosimilar were determined by commercial ELISA, and shown as mean+/−SEM. Results are shown in FIG. 92, which shows that free dexamethasone, when pre-dosed in the range of 1 to 40 mg/kg dose, each maintains IV DNA vectors already high level, long term protein production, as well as their ability to limit critical toxicity markers at or closely approximating background control levels. In addition, a number of the lowest free dexamethasone doses statistically significantly increased rituximab serum protein levels at day 1 following i.v. treatment.

TABLE 7

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| 0304-2F8 | Ab | SARS-CoV2 | | | SARS-CoV2 | S; Unk | B-cells; SARS-CoV2 | 63 | EVQLVQSGAEVSQPGESLKISCKGSGYSF | 1145 |
| | | (weak) | | | | | Human Patient | | TGYWISWVRQMPGKGLEWMGIIYPGD | |
| | | | | | | | | | SDTKYTPSFQGQVTISTDKSINTAYLQWS | |
| | | | | | | | | | SLKASDTAMYYCARRGDGLYYYGMDV | |
| | | | | | | | | | WGQGTTVTVSS | |
| 0304-3H3 | Ab | SARS-CoV2 | | SARS-CoV2 | | S; S2 | B-cells; SARS-CoV2 | 64 | EVQLVESGPGLVKPSETLSLTCTASGGSI | 1146 |
| | | | | | | | Human Patient | | STYYWSWIRQPPGKGLEWIGYIYYSGST | |
| | | | | | | | | | NYNPSLKSRVTISVDTSKNQFSLKLSSVTA | |
| | | | | | | | | | ADTAVYYCARDRIAPVGKFFGWYFDLW | |
| | | | | | | | | | GRGTLVTVSS | |
| 0304-4A10 | Ab | SARS-CoV2 | | | SARS-CoV2 | S; S2 | B-cells; SARS-CoV2 | 65 | EVQLVESGGGLVQPGGSLRLSCAASGFT | 1147 |
| | | | | | | | Human Patient | | FSTYAMHWVRQAPGKGLEYVSGISSNG | |
| | | | | | | | | | GSTYYANSVKGRFTISRDNSKNTLYIQM | |
| | | | | | | | | | GSLRAEDMAVYYCARSSSRGFDYWGQ | |
| | | | | | | | | | GTLVTVSS | |
| 0304-4A2 | Ab | SARS-CoV2 | | | SARS-CoV2 | S; S1 (non- | B-cells; SARS-CoV2 | 66 | EVQLVESGPGLVKPSETLSLTCAVSGDST | 1148 |
| | | | | | | RBD) | Human Patient | | SSSSSYWDWIRQPPGKGLEWIGNIYYTG | |
| | | | | | | | | | TTYYNPSLKSRVTISVDTSKDQFSLKLSSV | |
| | | | | | | | | | TAADTAVYYCARELFTAVAGKGGIDYW | |
| | | | | | | | | | GQGTLVTVSS | |
| 0304-A1 | Ab | SARS-CoV2 | | | SARS-CoV2 | S; S2 | B-cells; SARS-CoV2 | 67 | QVQLVQSGGGVVQPGRSLRLSCAAPGF | 1149 |
| | | | | | | | Human Patient | | TFSSYGMHWVRQAPGKGLEWVAVISY | |
| | | | | | | | | | DGSNKYYADSVKGRFTISRDNSKNTLYL | |
| | | | | | | | | | QMNSLRAEDTAVYYCAKDFKGGSSSWY | |
| | | | | | | | | | TPEIEYGMDVWGQGTTVTVSS | |
| 0317-A2 | Ab | SARS-CoV2 | | | SARS-CoV2 | S; S2 | B-cells; SARS-CoV2 | 68 | QVQLVQSGSELKKPGASVKVSCKASGYT | 1150 |
| | | | | | | | Human Patient | | FTSYAMNWVRQAPGQGLEWMGWINT | |
| | | | | | | | | | NTGNPTYAQGFTGRFVFSLDTSVSTAYL | |
| | | | | | | | | | QISSLKAEDTAVYYCARLIRHEAHTYCSG | |
| | | | | | | | | | GSCYSPDYYYGMDVWGQGTMVTVSS | |
| 0317-A3 | Ab | SARS-CoV2 | | | SARS-CoV2 | S; Unk | B-cells; SARS-CoV2 | 69 | EVQLLQSGGGLVQPGGSLRLSCVASGFT | 1151 |
| | | (weak) | | | | | Human Patient | | FSSYEMINWVRQAPGKGLEWVSYISTSG | |
| | | | | | | | | | STINYADSVKGRFSISRDNAKKSLYLQM | |
| | | | | | | | | | NSLRAEDTAVYYCASNPPLGEPYFDIWG | |
| | | | | | | | | | QGTMVTVSS | |
| 0317-A7 | Ab | SARS-CoV2 | | | SARS-CoV2 | S; S1 (non- | B-cells; SARS-CoV2 | 70 | EVQLLESGGGVVQPGRSLRLSCAASGFT | 1152 |
| | | | | | | RBD) | Human Patient | | FSNYAMHWVRQAPGKGLEWVAVISYD | |
| | | | | | | | | | GSNKYYADSVKGRFTISRDNSKNTLYLQ | |
| | | | | | | | | | MNSLRAEDTAVYYCARWGGGMQYLD | |
| | | | | | | | | | VWGQGTTVTVSS | |
| 0317-A8 | Ab | SARS-CoV2 | | | SARS-CoV2 | S; S1 (non- | B-cells; SARS-CoV2 | 71 | EVQLVQSGSELKKPGASVKVSCKASGYT | 1153 |
| | | (weak) | | | | RBD) | Human Patient | | FTSYAMINWVRQAPGQGLEWMGWINT | |
| | | | | | | | | | NTGNPTYAQGFTGRFVFSLDTSVSTAYL | |
| | | | | | | | | | QISSLKAEDTAVYYCARAGPNYDFWSGY | |
| | | | | | | | | | YQTFDYWGQGTMVTVSS | |
| 0317-A9 | Ab | SARS-CoV2 | | | SARS-CoV2 | S; Unk | B-cells; SARS-CoV2 | 72 | EVQLVQSGAEVKKPGASVKVSCKVSGYT | 1154 |
| | | (weak) | | | | | Human Patient | | LTELSMHWVRQAPGKGLEWMGGFDP | |
| | | | | | | | | | EDGETIYAQKFQGRVTMTEDTSTDTAY | |
| | | | | | | | | | MELSSLRSEDTAVYYCATATAMDGYYYY | |
| | | | | | | | | | YGMDVWGQGTTVTVSS | |
| 0317-B1 | Ab | SARS-CoV2 | | | SARS-CoV2 | S; Unk | B-cells; SARS-CoV2 | 73 | EVQLVESGAEVKKPGESLKISCKGSGYSF | 1155 |
| | | (weak) | | | | | Human Patient | | TSYWIGWVRQMPGEGLEWMGIIYPGD | |
| | | | | | | | | | SDTRYSPSFQGQVTISADKSISTAYLQWS | |
| | | | | | | | | | SLKASDTAMYYCASAGSSWYGDAFDIW | |
| | | | | | | | | | GQGTMVTVSS | |
| 0317-C4 | Ab | SARS-CoV2 | | | SARS-CoV2 | S; Unk | B-cells; SARS-CoV2 | 74 | QVQLVQSGAEVKKPGASVKVSCKVSGC | 1156 |
| | | (weak) | | | | | Human Patient | | TLTELSMHWVRQAPGKGLEWMGGFD | |
| | | | | | | | | | PEDGETIYAQKFQGRVTMTEDTSTDTAY | |
| | | | | | | | | | MELSSLRSEDTAVYYCATATIFGVANNW | |
| | | | | | | | | | FDPWGQGTTVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| 0304-2F8 | EIVMTQSHTLLPVTPGEPASITCRSSQS | IGHV5-51 | IGHJ6 | IGKV2-28 | IGKJ3 | 2057 | ARRGDGLYYY | 3266 | MQALQTP | Xiangyang Chi et al., |
| | LLHSNGYNYLDWYLQKPGQSPQLLIYL | (Human) | (Human) | (Human) | (Human) | | GMDV | | QT | 2020 |
| | GSNRASGVPDRFSGSGSGTDFTLKISR | | | | | | | | | (https://science.science |
| | VEAEDVGVYYCMQALQTPQTFGQGT | | | | | | | | | mag.org/content/early/ |
| | KVDIK | | | | | | | | | 2020/06/19/science.abc |
| | | | | | | | | | | 6952/tab-pdf) |
| 0304-3H3 | DIVMTQSPATLSVSPEERATLSCRASQ | IGHV4-59 | IGHJ2 | IGKV3-15 | IGKJ1 | 2058 | ARDRIAPVGK | 3267 | QQYNKWP | Xiangyang Chi et al., |
| | SVSSNLAWYQQKPGQAPRLLIYGAST | (Human) | (Human) | (Human) | (Human) | | FFGWYFDL | | PWT | 2020 |
| | RATGIPARFSGSGSGTEFTLTISSLQSE | | | | | | | | | (https://science.science |
| | DFAVYYCQQYNKWPPWTFGQGTKV | | | | | | | | | mag.org/content/early/ |
| | DIK | | | | | | | | | 2020/06/19/science.abc |
| | | | | | | | | | | 6952/tab-pdf) |
| 0304-4A10 | EIVLTQSPDSLAVSLGERATINCRSSQS | IGHV3-64 | IGHJ4 | IGKV4-1 | IGKJ3 | 2059 | ARSSSRGFDY | 3268 | QQYYSSPY | Xiangyang Chi et al., |
| | VLYSSNNKNYLAWYQQKPGQPPKVLI | (Human) | (Human) | (Human) | (Human) | | | | A | 2020 |
| | YWASTRESGVPDRFSGSGSGTDFTLTI | | | | | | | | | (https://science.science |
| | SSLQAEDVAVYYCQQYYSSPYAFGPGT | | | | | | | | | mag.org/content/early/ |
| | KVDIK | | | | | | | | | 2020/06/19/science.abc |
| | | | | | | | | | | 6952/tab-pdf) |
| 0304-4A2 | EIVLTQSPDSLAVSLGERATINCKSSQS | IGHV4-39 | IGHJ4 | IGKV4-1 | IGKJ1 | 2060 | ARELFTAVAG | 3269 | HQYYNTPR | Xiangyang Chi et al., |
| | VLYSSNNKNYLAWYQQKPGQPPKLLI | (Human) | (Human) | (Human) | (Human) | | KGGIDY | | T | 2020 |
| | YWASTRESGVPDRFSGSGSGTDFTLTI | | | | | | | | | (https://science.science |
| | SSLQAEDVAVYYCHQYYNTPRTFGQG | | | | | | | | | mag.org/content/early/ |
| | TKVEIK | | | | | | | | | 2020/06/19/science.abc |
| | | | | | | | | | | 6952/tab-pdf) |
| 0304-A1 | DIVMTQSPATLSLSPGERATLSCRASQ | (Human) | IGHJ6 | IGKV3-11 | IGKJ3 | 2061 | AKDFKGGSSS | 3270 | RQRSNWP | Xiangyang Chi et al., |
| | SVSSYLAWYQQKPGQAPRLLIYDASN | IGHV3-30 | (Human) | (Human) | (Human) | | WYTPEIEYG | | PT | 2020 |
| | RATGIPARFSGSGSGADFTLTISSLEPE | | | | | | MDV | | | (https://science.science |
| | DFAVYYCRQRSNWPPTFGGGTKVDIK | | | | | | | | | mag.org/content/early/ |
| | | | | | | | | | | 2020/06/19/science.abc |
| | | | | | | | | | | 6952/tab-pdf) |
| 0317-A2 | DIVMTQSPSSLSASVGDRVTITCRASQ | IGHV7-4- | IGHJ6 | IGKV1-39 | IGKJ3 | 2062 | ARLIRHEAHT | 3271 | QQSYSTPP | 2020/06/19/science.abc |
| | SISSYLNWYQQEPGKAPKLLIYAASSLQ | 1 | (Human) | (Human) | (Human) | | YCSGGSCYSP | | T | Xiangyang Chi et al., |
| | SGVPSRFSGSGSGTDFTLTISSLQPEDF | (Human) | | | | | DYYYGMDV | | | 2020 |
| | ATYYCQQSYSTPPTFGQGTKVDIK | | | | | | | | | (https://science.science |
| | | | | | | | | | | mag.org/content/early/ |
| | | | | | | | | | | 6952/tab-pdf) |
| 0317-A3 | DIQMTHSLILLSASVGDRVTITCRASQS | IGHV3-48 | IGHJ3 | IGKV1-39 | IGKJ1 | 2063 | ASNPPLGEPY | 3272 | QQTYRPP | Xiangyang Chi et al., |
| | ISSYLNWYQQKPGKAPKLLIYAASSLQS | (Human) | (Human) | (Human) | (Human) | | FDI | | WT | 2020 |
| | GVPSRFSGSGSGADFTLTISSLQPEDFA | | | | | | | | | (https://science.science |
| | TYYCQQTYRPPWTFGQGTKVDIK | | | | | | | | | mag.org/content/early/ |
| | | | | | | | | | | 2020/06/19/science.abc |
| | | | | | | | | | | 6952/tab-pdf) |
| 0317-A7 | EIVVTQSLLSLPVTPGEPASISCRSSQSL | IGHV3-30- | IGHJ6 | IGKV2-28 | IGKJ1 | 2064 | ARWGGGMQ | 3273 | MQTLQTP | Xiangyang Chi et al., |
| | LHSNGYNYLDWYLQKPGQSPQVLIYL | 3 | (Human) | (Human) | (Human) | | YLDV | | YT | 2020 |
| | GSNRASGVPDRFSGSGSGTDFTLKISR | (Human) | | | | | | | | (https://science.science |
| | VEAEDVGVYYCMQTLQTPYTFGQGTK | | | | | | | | | mag.org/content/early/ |
| | VEIK | | | | | | | | | 2020/06/19/science.abc |
| | | | | | | | | | | 6952/tab-pdf) |
| 0317-A8 | DIQMTQSPSSLSASVGDRVTITCQASQ | IGHV7-4- | IGHJ4 | IGKV1-33 | IGKJ4 | 2065 | ARAGPNYDF | 3274 | QQYDNLPL | Xiangyang Chi et al., |
| | DISNYLNWYQQKPGKAPKLLIYDASNL | 1 | (Human) | (Human) | (Human) | | WSGYYQTFD | | T | 2020 |
| | ETGVPSRFSGSGSGTDFTFTISSLQPED | (Human) | | | | | Y | | | (https://science.science |
| | IATYYCQQYDNLPLTFGGGTKVEIK | | | | | | | | | mag.org/content/early/ |
| | | | | | | | | | | 2020/06/19/science.abc |
| | | | | | | | | | | 6952/tab-pdf) |
| 0317-A9 | EIVMTQSPLSSPVTLGQPASISCRSSQS | IGHV1-24 | IGHJ6 | IGKV2-24 | IGKJ3 | 2066 | ATATAMDGY | 3275 | MQATQFP | Xiangyang Chi et al., |
| | LVHSDGNTYLSWLQQRPGQPPRLLIY | (Human) | (Human) | (Human) | (Human) | | YYYYGMDV | | YT | 2020 |
| | KISNRFSGVPDRFSGSGAGTDFTLKISR | | | | | | | | | (https://science.science |
| | VEAEDVGVYYCMQATQFPYTFGQGT | | | | | | | | | mag.org/content/early/ |
| | KVDIK | | | | | | | | | 2020/06/19/science.abc |
| | | | | | | | | | | 6952/tab-pdf) |
| 0317-B1 | DIVMTHSLRLLAVSLGERATINCKSSQS | IGHV5-51 | IGHJ3 | IGKV4-1 | IGKJ3 | 2067 | ASAGSSWYG | 3276 | QQYYSTYG | Xiangyang Chi et al., |
| | VLYSSNNKNYLAWYQQKPGQPPKLLI | (Human) | (Human) | (Human) | (Human) | | DAFDI | | S | 2020 |
| | YWASTRESGVPDRFSGSGSGTDFTLTI | | | | | | | | | (https://science.science |
| | SSLQAEDVAVYYCQQYYSTYGSFGGG | | | | | | | | | mag.org/content/early/ |
| | TKVDIK | | | | | | | | | 2020/06/19/science.abc |
| | | | | | | | | | | 6952/tab-pdf) |
| 0317-C4 | QAVVTQPPSVSAAPGQKVTISCSGSSS | IGHV1-24 | IGHJ5 | IGLV1-51 | IGLJ3 | 2068 | ATATIFGVAN | 3277 | GTWDSSLS | Xiangyang Chi et al., |
| | NIGNNYVSWYQQLPGTAPKLLIYDNN | (Human) | (Human) | (Human) | (Human) | | NWFDP | | VVV | 2020 |
| | KRPSGIPDRFSGSKSGTSATLGITGLQT | | | | | | | | | (https://science.science |
| | GDEADYYCGTWDSSLSVVVFGGGTKL | | | | | | | | | mag.org/content/early/ |
| | TVL | | | | | | | | | 2020/06/19/science.abc |
| | | | | | | | | | | 6952/tab-pdf) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| 0317-C9 | Ab | SARS-CoV2 | | | SARS-CoV2 | S; S2 | B-cells; SARS-CoV2 | 75 | QVQLVQSGGGVVQPGRSLRLSCAASGF | 1157 |
| | | | | | | | Human Patient | | TFSSYGMHWVRQAPGKGLEWVAVISY | |
| | | | | | | | | | DGSNKYYADSVKGRFTISRDNSKNTLYL | |
| | | | | | | | | | QMNSLRAEDTAVYYCAKDLGYYDILTGQ | |
| | | | | | | | | | LGGYYYYYGMDVWGQGTTVTVSS | |
| 10C10 | Ab | SARS-CoV2 | | | SARS-CoV2 | S; Unk | B-cells; SARS-CoV2 | 76 | QVQLVQSGGGLVQPGGSLRLSCAASGF | 1158 |
| | | | | | | | Human Patient | | TFSSYWMSWVRQAPGKGLEWVANIN | |
| | | | | | | | | | QDGSEKYYVDSVKGRFTISRDNAKNSLY | |
| | | | | | | | | | LQMNSLRAEDTAVYYCARDWDYDILTG | |
| | | | | | | | | | SWFGAFDIWGQGTMVTVSS | |
| 1A09 | Ab | SARS-CoV2 | SARS- | | SARS-CoV2 | S; RBD | Immunised Mouse | 77 | QVQLKESGPGLVAPSQSLSITCTVSGFSL | 1159 |
| | | | CoV1 | | | | | | TSYAISWVRQPPGKGLEWLGVIWTGGG | |
| | | | | | | | | | TNYNSALKSRLSISKDNSKSQVFLKMNSL | |
| | | | | | | | | | QTDDTARYYCARKDYYYGSSYAMDYWG | |
| | | | | | | | | | QGTSVTVSS | |
| 1A10 | Ab | SARS-CoV2 | MERS- | | SARS-CoV2 | S; RBD | Immunised Mouse | 78 | EVQLQQSGAELVRPGASVKLSCKASGYT | 1160 |
| | | | CoV, | | | | | | FSDYYINWVKQRPGQGLEWIARIYPGS | |
| | | | SARS- | | | | | | GNTYYNEKFTGKATLTAEKSSSTAYMQL | |
| | | | CoV1 | | | | | | SSLTSEDSAVYFCARDYGSSYVDYFDYW | |
| | | | | | | | | | GQGTTLTVSS | |
| 1A12 | Ab | SARS-CoV2 | MERS- | SARS-CoV2 | | S; RBD | Immunised Mouse | 79 | EVQLQQSGAELVRPGASVKLSCTASGEN | 1161 |
| | | | CoV, | (weak) | | | | | IKDDYMHWVKQRPEQGLEWIGWINPE | |
| | | | SARS- | | | | | | NGDTEYASKFQGKAAITADTSSNTACLQ | |
| | | | CoV1 | | | | | | LSSLTSEDTAVYYCSTGGYGNYVDAMDY | |
| | | | | | | | | | WGQGTSVTVSS | |
| 1B07 | Ab | SARS-CoV2 | MERS- | SARS-CoV2 | | S; RBD | Immunised Mouse | 80 | QVQLKESGPGLVAPSQSLSITCTVSGFSL | 1162 |
| | | | CoV, | | | | | | TSYAISWVRQPPGKGLEWLGVIWTGGG | |
| | | | SARS- | | | | | | TNYNSALKSRLSISKDNSKSQVFLKMNSL | |
| | | | CoV1 | | | | | | QTDDTARYYCARKDYYGSSSNVMDYW | |
| | | | | | | | | | GQGTSVTVSS | |
| 1B10 | Ab | SARS-CoV2 | MERS- | SARS-CoV2 | | S; RBD | Immunised Mouse | 81 | QVQLKESGPGLVAPSQSLSITCTVSGFSL | 1163 |
| | | | CoV, | | | | | | TNYAINWVRQPPGKGLEWLGVIWTGG | |
| | | | SARS- | | | | | | GTNYNSALRSRLSISKDNSKSQVFLKMN | |
| | | | CoV1 | | | | | | SLHTDDTARYYCARKDYYGSSLAMDYW | |
| | | | | | | | | | GQGTSVTVSS | |
| 1C05 | Ab | SARS-CoV1, | MERS- | SARS-CoV2 | | S; RBD | Immunised Mouse | 82 | EVQLQQSGPELVKPGASVKISCKASGYS | 1164 |
| | | SARS-CoV2 | CoV | (weak) | | | | | FTDYYMNWVKQSPEKSLEWIGEINPST | |
| | | | | | | | | | GGPTYNQKFKAKATVTVDKSSSTAYMQ | |
| | | | | | | | | | LKSLTSEDSAVYYCARRNYDLYYYAMIDY | |
| | | | | | | | | | WGQGTSVTVSS | |
| 1C06 | Ab | SARS-CoV2 | MERS- | | SARS-CoV2 | S; RBD | Immunised Mouse | 83 | EVQLQQSGAELAKPGASVKLSCKASGYT | 1165 |
| | | | CoV, | | | | | | FTNYWMHWVKQRPGQGLEWIGYINPS | |
| | | | SARS- | | | | | | SGYTKFNQKFNDKATLTADKSSTTAYMQ | |
| | | | CoV1 | | | | | | LSSPTYEDSAVYYCARSDYYGSSYVGYA | |
| | | | | | | | | | MDYWGQGTSVTVSS | |
| 1C07 | Ab | SARS-CoV1, | MERS- | | SARS-CoV2 | S; RBD | Immunised Mouse | 84 | EVQLQQPGPELVKPGASVKISCKASGYS | 1166 |
| | | SARS-CoV2 | CoV | | | | | | FTDYYMNWVKQSPEKSLEWIGEINPST | |
| | | | | | | | | | GGTSYNQKFKGKATLTVDKSSSTAYMQL | |
| | | | | | | | | | KSLTSEDSAVYYCARRNYDLYYYAMDYW | |
| | | | | | | | | | GQGTSVTVSS | |
| 1D04 | Ab | SARS-CoV2 | MERS- | | SARS-CoV2 | S; RBD | Immunised Mouse | 85 | EVQLQQSGAELVRPGASVKLSCTASGEN | 1167 |
| | | | CoV, | | | | | | IKDDYMHWVKQRPEQGLEWIGWINPE | |
| | | | SARS- | | | | | | NGDTEYASKFQGKAAITADTSSNTACLQ | |
| | | | CoV1 | | | | | | LSSLTSEDTAVYYCSTGGYGNYVDAMDY | |
| | | | | | | | | | WGQGTSVTVSS | |
| 1D05 | Ab | SARS-CoV2 | MERS- | | SARS-CoV2 | S; RBD | Immunised Mouse | 86 | QVQLKESGPGLVAPSQSLSITCTVSGFSL | 1168 |
| | | | CoV, | | | | | | TSYAISWVRQPPGKGLEWLGVIWTGGG | |
| | | | SARS- | | | | | | TNYNSALKSRLSISKDNSKSQVFLKMNSL | |
| | | | CoV1 | | | | | | QTDDTARYYCARKDYYGSSSNVMDYW | |
| | | | | | | | | | GQGTSVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| 0317-C9 | QAVVTQPPSASGTPGQRVTISCSGSSS | IGHV3-30 | IGHJ6 | IGLV1-44 | IGLJ3 | 2069 | AKDLGYYDILT | 3278 | AAWDDSL | Xiangyang Chi et al., |
| | NIGSNTVNWYQQLPGTAPKLLIYSNN | (Human) | (Human) | (Human) | (Human) | | GQLGGYYYYY | | NGVV | 2020 |
| | QRPSGVPDRFSGSKSGTSASLAISGLQ | | | | | | GMDV | | | (https://science.science |
| | SEDEADYYCAAWDDSLNGVVFGGGT | | | | | | | | | mag.org/content/early/ |
| | KLTVL | | | | | | | | | 2020/06/19/science.abc |
| | | | | | | | | | | 6952/tab-pdf) |
| 10C10 | DIQLTQSPSSLSASVRNRVTITCRASQG | IGHV3-7 | IGHJ3 | IGKV1-17 | IGKJ4 | 2070 | ARDWDYDILT | 3279 | LQHNNYPL | Xiangyang Chi et al., |
| | IKNDLCWYQQKPGKAPKRLIYAASSLQ | (Human) | (Human) | (Human) | (Human) | | GSWFGAFDI | | T | 2020 |
| | SGVPSRFSGSGSGTEFTLTISSLQPEDF | | | | | | | | | (https://science.science |
| | ATYYCLQHNNYPLTFGGGTNEEIK | | | | | | | | | mag.org/content/early/ |
| | | | | | | | | | | 2020/06/19/science.abc |
| | | | | | | | | | | 6952/tab-pdf) |
| 1A09 | QAVVTQESALTTSPGETVTLTCRSSTG | IGHV2-9- | IGHJ4 | IGLV1 | IGLJ1 | 2071 | ARKDYYYGSS | 3280 | ALWYSNH | Wafaa Alsoussi et al., |
| | AVTTSNYANWVQEKPDHLFTGLIGGT | 1 | (Mouse) | (Mouse) | (Mouse) | | YAMDY | | WV | 2020 |
| | NNRAPGVPARFSGSLIGDKAALTITGA | (Mouse) | | | | | | | | (https://www.jimmunol |
| | QTEDEAIYFCALWYSNHWVFGGGTKL | | | | | | | | | .org/content/early/202 |
| | TVL | | | | | | | | | 0/06/23/jimmunol.2000 |
| | | | | | | | | | | 583) |
| 1A10 | DIQMTQSPSSLSASLGDRVTISCRASQ | IGHV1-76 | IGHJ2 | IGKV10-96 | IGKJ2 | 2072 | ARDYGSSYVD | 3281 | QQGNTLP | Wafaa Alsoussi et al., |
| | DISNYLNWYQQKPDGTVKLLIFYTSRL | (Mouse) | (Mouse) | (Mouse) | (Mouse) | | YFDY | | YT | 2020 |
| | HSGVPSRFSGSGSGTDYSLTISNLEQE | | | | | | | | | (https://www.jimmuno |
| | DIATYFCQQGNTLPYTFGGGTKLEIK | | | | | | | | | org/content/early/202 |
| | | | | | | | | | | 0/06/23/jimmunol.2000 |
| | | | | | | | | | | 583) |
| 1A12 | DIKMTQSPSSMYASLGERVTITCKASQ | IGHV14-4 | IGHJ4 | IGKV14- | IGKJ1 | 2073 | STGGYGNYV | 3282 | LQYDEFRT | Wafaa Alsoussi et al., |
| | DINSFLSWFQQKPGKSPKTLIYRANRL | (Mouse) | (Mouse) | 111 | (Mouse) | | DAMDY | | | 2020 |
| | VDGVPSRFSGSGSGQDYSLTISSLDYE | | | (Mouse) | | | | | | (https://www.jimmunol |
| | DMGIYYCLQYDEFRTFGGGTKLEIK | | | | | | | | | .org/content/early/202 |
| | | | | | | | | | | 0/06/23/jimmunol.2000 |
| | | | | | | | | | | 583) |
| 1B07 | QAVVTQESALTTSPGETVTLTCRSSTG | IGHV2-9- | IGHJ4 | IGLV1 | IGLJ3 | 2074 | ARKDYYGSSS | 3283 | ALWYSNQ | Wafaa Alsoussi et al., |
| | AVTTSNYANWVQEKPDHLITGLIGGT | 1 | (Mouse) | (Mouse) | (Mouse) | | NVMDY | | F | 2020 |
| | NNRVPGVPARFSGSLIGDKAALTITGA | (Mouse) | | | | | | | | (https://www.jimmunol |
| | QTEDEAIYFCALWYSNQFIFGSGTKVT | | | | | | | | | .org/content/early/202 |
| | VL | | | | | | | | | 0/06/23/jimmunol.2000 |
| | | | | | | | | | | 583) |
| 1B10 | QAVVTQESALTTSPGETVTLTCRSSTG | IGHV2-9- | IGHJ4 | IGLV1 | IGLJ1 | 2075 | ARKDYYGSSL | 3284 | ALWYSNH | Wafaa Alsoussi et al., |
| | AVTTSNYANWVQEKPDHLFTGLIGGT | (Mouse) | (Mouse) | (Mouse) | (Mouse) | | AMDY | | WV | 2020 |
| | NNRAPGVPARFSGSLIGDKAALTITGA | | | | | | | | | (https://www.jimmunol |
| | QTEDEAIYFCALWYSNHWVFGGGTKL | | | | | | | | | .org/content/early/202 |
| | TVL | | | | | | | | | 0/06/23/jimmunol.2000 |
| | | | | | | | | | | 583) |
| 1C05 | DIVLTQSPASLAVSLGQRATISCRASES | IGHV1-42 | IGHJ4 | IGKV3-10 | IGKJ5 | 2076 | ARRNYDLYYY | 3285 | QQNNEDP | Wafaa Alsoussi et al., |
| | VDSYGTSFMHWYQQKPGQPPKLLIYL | (Mouse) | (Mouse) | (Mouse) | (Mouse) | | AMDY | | LT | 2020 |
| | ASNLESGVPARFSGSGSRTDFTLTIDPV | | | | | | | | | (https://www.jimmunol |
| | EADDAATYYCQQNNEDPLTFGAGTKL | | | | | | | | | .org/content/early/202 |
| | ELK | | | | | | | | | 0/06/23/jimmunol.2000 |
| | | | | | | | | | | 583) |
| 1C06 | DVVMTQTPLSLPVSLGDQASISCRSSQ | IGHV1-7 | IGHJ4 | IGKV1-117 | IGKJ1 | 2077 | ARSDYYGSSY | 3286 | FQGSHVPP | Wafaa Alsoussi et al., |
| | SIVHSNGNTYLEWYLQKPGQSPKLLIY | (Mouse) | (Mouse) | (Mouse) | (Mouse) | | VGYAMDY | | T | 2020 |
| | KVSNRFSGVPDRFSGSGSGTDFTLKIS | | | | | | | | | (https://www.jimmunol |
| | RVEAEDLGVYYCFQGSHVPPTFGGGT | | | | | | | | | .org/content/early/202 |
| | KLEIK | | | | | | | | | 0/06/23/jimmunol.2000 |
| | | | | | | | | | | 583) |
| 1C07 | DIVLTQSPASLAVSLGQRATISCRASEG | IGHV1-42 | IGHJ4 | IGKV3-10 | IGKJ5 | 2078 | ARRNYDLYYY | 3287 | QQNNEDP | Wafaa Alsoussi et al., |
| | VDSYGSSFMHWYQQKPGQPPKLLIYL | (Mouse) | (Mouse) | (Mouse) | (Mouse) | | AMDY | | LT | 2020 |
| | ASNLESGVPARFSGSGSGTDFTLTIDP | | | | | | | | | (https://www.jimmunol |
| | VEADDAATYYCQQNNEDPLTFGAGTK | | | | | | | | | .org/content/early/202 |
| | LELK | | | | | | | | | 0/06/23/jimmunol.2000 |
| | | | | | | | | | | 583) |
| 1D04 | DIKMITQSPSSMYASLGERVTITCKASQ | IGHV14-4 | IGHJ4 | IGKV14- | IGKJ1 | 2079 | STGGYGNYV | 3288 | LQYDEFRT | Wafaa Alsoussi et al., |
| | DINSFLSWFQQKPGKSPKTLIYRANRL | (Mouse) | (Mouse) | 111 | (Mouse) | | DAMDY | | | 2020 |
| | VDGVPSRFSGSGSGQDYSLTISSLEYED | | | (Mouse) | | | | | | (https://www.jimmunol |
| | MGIYYCLQYDEFRTFGGGTKLEIK | | | | | | | | | .org/content/early/202 |
| | | | | | | | | | | 0/06/23/jimmunol.2000 |
| | | | | | | | | | | 583) |
| 1D05 | QAVVTQESALTTSPGETVTLTCRSSTG | IGHV2-9- | IGHJ4 | IGLV1 | IGLJ3 | 2080 | ARKDYYGSSS | 3289 | ALWYSNQ | Wafaa Alsoussi et al., |
| | AVTTSNYANWVQEKPDHLITGLIGGT | 1 | (Mouse) | (Mouse) | (Mouse) | | NVMDY | | FI | 2020 |
| | NNRVPGVPARFSGSLIGDKAALTITGA | (Mouse) | | | | | | | | (https://www.jimmunol |
| | QTEDKAIYFCALWYSNQFIFGSGTKVT | | | | | | | | | .org/content/early/202 |
| | VL | | | | | | | | | 0/06/23/jimmunol.2000 |
| | | | | | | | | | | 583) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| 1E02 | Ab | SARS-CoV2 | MERS- | | SARS-CoV2 | S; RBD | Immunised Mouse | 87 | EVQLQQPGPELVKPGASVKISCKASGYS | 1169 |
| | | | CoV, | | | | | | FTDYYMNWVKQSPEKSLEWIGEINPST | |
| | | | SARS- | | | | | | GGTSYNQKFKGKATLTVDKSSSTAYMQL | |
| | | | CoV1 | | | | | | KSLTSEDSAVYYCSRRNYDLYYYAMDYW | |
| | | | | | | | | | GQGTSVTVSS | |
| 1E07 | Ab | SARS-CoV2 | MERS- | SARS-CoV2 | | S; RBD | Immunised Mouse | 88 | EVQLQQSGPELVKPGASVKISCKASGYS | 1170 |
| | | | CoV, | | | | | | FTGYSMNWVKKSPEKSLEWIGEINPSTG | |
| | | | SARS- | | | | | | GTTYNQKFKAKATLTVDKSSSTAYIQLKS | |
| | | | CoV1 | | | | | | LTSEDSAVYYCARGAGAYWGQGTLVTV | |
| | | | | | | | | | SA | |
| 1E10 | Ab | SARS-CoV2 | MERS- | | SARS-CoV2 | S; RBD | Immunised Mouse | 89 | EVQLQQSGPELVKPGASVKISCKASGYA | 1171 |
| | | | CoV, | | | | | | FSSSWMNWVKQRPGKGLEWIGRIYPG | |
| | | | SARS- | | | | | | DGDTNYNGKFKGKATLTADKSSSTAYM | |
| | | | CoV1 | | | | | | QLSSLTSEDSAVYFCARDHGPAWFAYRG | |
| | | | | | | | | | QGTLVTVSA | |
| 1H06 | Ab | SARS-CoV2 | MERS- | | SARS-CoV2 | S; RBD | Immunised Mouse | 90 | EVQLQQSGPELVKPGASVKISCKASGYS | 1172 |
| | | | CoV, | | | | | | FTGYSMNWVKKSPEKSLEWIGEINPSTG | |
| | | | SARS- | | | | | | GTTYNQKFKAKATLTVDKSSSTAYIQLKS | |
| | | | CoV1 | | | | | | LTSEDSAVYYCARGAGAYWGQGTLVTV | |
| | | | | | | | | | SA | |
| 1H10 | Ab | SARS-CoV2 | MERS- | SARS-CoV2 | | S; RBD | Immunised Mouse | 91 | QVQLKESGPGLVAPSQSLSITCTVSGFSL | 1173 |
| | | | CoV, | (weak) | | | | | TNYAISWVRQPPGKGLEWLGVIWTGG | |
| | | | SARS- | | | | | | GTNYNSALKSRLSISKDNSKSQVFLKMN | |
| | | | CoV1 | | | | | | SLQTDDTARYYCARISYYDYEGVDYWGQ | |
| | | | | | | | | | GTTLTVSS | |
| 1M-1D2 | Ab | SARS-CoV2 | | SARS-CoV2 | | S; RBD | B-cells; SARS-CoV2 | 92 | QVQLVESGGGLVQPGGSLRLSCAASGF | 1174 |
| | | | | | | | Human Patient | | TFSSYAMHWVRQAPGKGLEYVSAISGN | |
| | | | | | | | | | GGSTYYAKSVKGRFTISRDNSKNTLYLQ | |
| | | | | | | | | | MGSLRAEDMAVYYCARGAEYYDFWSG | |
| | | | | | | | | | YYSAYFDYWGQGTLVTVSS | |
| 2B04 | Ab | SARS-CoV2 | MERS- | SARS-CoV2 | | S; RBD | Immunised Mouse | 93 | QVQLKQSGPGLVAPSQSLSITCTVSGFSL | 1175 |
| | | | CoV, | | | | | | INYAISWVRQPPGKGLEWLGVIWTGGG | |
| | | | SARS- | | | | | | TNYNSALKSRLSISKDNSKSQVFLKMNSL | |
| | | | CoV1 | | | | | | QTDDTARYYCARKDYYGRYYGMDYWG | |
| | | | | | | | | | QGTSVTVSS | |
| 2C02 | Ab | SARS-CoV1, | MERS- | | SARS-CoV2 | S; RBD | Immunised Mouse | 94 | EVQLQQPGAELVKPGASVKVSCKASGYI | 1176 |
| | | SARS-CoV2 | CoV | | | | | | FTNYWMHWVKQRPGQGLEWIGRIHPS | |
| | | | | | | | | | DSDTKYNQKFKGKATLTVDKSSSTAYMQ | |
| | | | | | | | | | LSSLTSEDSAVYYCAILDSYWYFDVWGT | |
| | | | | | | | | | GTTVTVSS | |
| 2C03 | Ab | SARS-CoV2 | MERS- | SARS-CoV2 | | S; RBD | Immunised Mouse | 95 | EVQLQQSGPELVKPGASVKISCKASGYA | 1177 |
| | | | CoV, | (weak) | | | | | FSSSWMNWVKQRPGKGLEWIGRIYPG | |
| | | | SARS- | | | | | | DGDTNYNGKLKGKATLTADKSSSTAYM | |
| | | | CoV1 | | | | | | QLSSLTSEDSAVYFCARKSYGYWHFDV | |
| | | | | | | | | | WGTGTTVTVSS | |
| 2C04 | Ab | SARS-CoV2 | MERS- | | SARS-CoV2 | S; RBD | Immunised Mouse | 96 | EVQLQQPGPELVKPGTSVKISCKASGYT | 1178 |
| | | | CoV, | | | | | | FTDYYMNWVKQSHGKSLEWIGDINPN | |
| | | | SARS- | | | | | | NGGTNYNQKFKGKATLTVDKSSSTAYM | |
| | | | CoV1 | | | | | | ELRSLTSEDSAVCYCAAGKGDYWGQGT | |
| | | | | | | | | | TLTVSS | |
| 2D01 | Ab | SARS-CoV2 | MERS- | | SARS-CoV2 | S; RBD | Immunised Mouse | 97 | EVQLQQPGAELVKPGASVKVSCKASGYI | 1179 |
| | | | CoV, | | | | | | FTNYWMHWVKQRPGQGLEWIGRIHPS | |
| | | | SARS- | | | | | | DSDTKYNQKFKGKATLTVDKSSSTAYMQ | |
| | | | CoV1 | | | | | | LSSLTSEDSAVYYCAILDSYWYFDVWGT | |
| | | | | | | | | | GTTVTVSS | |
| 2D08 | Ab | SARS-CoV2 | MERS- | SARS-CoV2 | | S; RBD | Immunised Mouse | 98 | EVQLQQSGPELVKPGTSVKISCKASGYTF | 1180 |
| | | | CoV, | (weak) | | | | | TDYYMNWVKQSHGKSLEWIGDINPNN | |
| | | | SARS- | | | | | | GGTNYNQKFKGKATLTVDKSSSTAYMEL | |
| | | | CoV1 | | | | | | RSLTSEDSAVYYCAAGKGDYWGQGTTL | |
| | | | | | | | | | TVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| 1E02 | DIVLTQSPASLAVSLGQRATISCRASEG | IGHV1-42 | IGHJ4 | IGKV3-10 | IGKJ5 | 2081 | SRRNYDLYYY | 3290 | QQNNEDP | Wafaa Alsoussi et al., |
| | VDSYGSSFMHWYQQKPGQAPKLLIYL | (Mouse) | (Mouse) | (Mouse) | (Mouse) | | AMDY | | LT | 2020 |
| | ASNLESGVPARFSGSGSRTDFTLTIDPV | | | | | | | | | (https://www.jimmunol |
| | EADDAAIYYCQQNNEDPLTFGAGTKL | | | | | | | | | org/content/early/202 |
| | EIK | | | | | | | | | 0/06/23/jimmunol.2000 |
| | | | | | | | | | | 583) |
| 1E07 | DIVLTQSPASLAVSLGQRATISCRASES | IGHV1-42 | IGHJ3 | IGKV3-1 | IGKJ1 | 2082 | ARGAGAY | 3291 | QQSRKVP | Wafaa Alsoussi et al., |
| | VEYYGTSLMQWFQQKPGQPPKLLIFA | (Mouse) | (Mouse) | (Mouse) | (Mouse) | | | | WT | 2020 |
| | ASNVESGVPARFGGSGSGTDFSLNIHP | | | | | | | | | (https://www.jimmunol |
| | VEEDDIAMYFCQQSRKVPWTFGGGT | | | | | | | | | .org/content/early/202 |
| | KLEIK | | | | | | | | | 0/06/23/jimmunol.2000 |
| | | | | | | | | | | 583) |
| 1E10 | QIVLTQSPAIMSASPGEKVTMTCSASS | IGHV1-82 | IGHJ3 | IGKV4-59 | IGKJ2 | 2083 | ARDHGPA | 3292 | QQWSSNP | Wafaa Alsoussi et al., |
| | SVSYMHWYQQKSGTSPKRWIYDTSKL | (Mouse) | (Mouse) | (Mouse) | (Mouse) | | | | PT | 2020 |
| | ASGVPARFSGSGSGTSYSLTISSMEAE | | | | | | | | | (https://www.jimmunol |
| | DAATYYCQQWSSNPPTFGSGTKLEIK | | | | | | | | | .org/content/early/202 |
| | | | | | | | | | | 0/06/23/jimmunol.2000 |
| | | | | | | | | | | 583)) |
| 1H06 | DIVLTQSPASLAVSLGQRATISCRASES | IGHV1-42 | IGHJ3 | IGKV3-1 | IGKJ1 | 2084 | ARGAGAY | 3293 | QQSRKVP | Wafaa Alsoussi et al., |
| | VEYYGTSLMQWFQQKPGQPPKLLIFA | (Mouse) | (Mouse) | (Mouse) | (Mouse) | | | | WT | 2020 |
| | ASNVESGVPARFGGSGSGTDFSLNIHP | | | | | | | | | (https://www.jimmunol |
| | VEEDDIATYFCQQSRKVPWTFGGGTK | | | | | | | | | .org/content/early/202 |
| | LEIK | | | | | | | | | 0/06/23/jimmunol.2000 |
| | | | | | | | | | | 583 |
| 1H10 | QAVVTQESALTTSPGETVTLTCRSSTG | IGHV2-9- | IGHJ2 | IGLV1 | IGLJ1 | 2085 | ARISYYDYEG | 3294 | ALWYSNH | Wafaa Alsoussi et al., |
| | AVTTSNYANWVQEKPDHLFTGLIGGT | 1 | (Mouse) | (Mouse) | (Mouse) | | VDY | | WV | 2020 |
| | NNRAPGVPARFSGSLIGDKAALTITGA | (Mouse) | | | | | | | | (https://www.jimmunol |
| | QTEDEAIYFCALWYSNHWVFGGGTKL | | | | | | | | | .org/content/early/202 |
| | TVL | | | | | | | | | 0/06/23/jimmunol.2000 |
| | | | | | | | | | | 583) |
| 1M-1D2 | QAVVTQPPSASGTPGQRVTISCSGSSS | IGHV3-64 | IGHJ4 | IGLV1-47 | IGLJ3 | 2086 | ARGAEYYDF | 3295 | AAWDDSLI | Xiangyang Chi et al., |
| | NIGSNYVYWYQQLPGTAPKLLIYNNN | (Human) | (Human) | (Human) | (Human) | | WSGYYSAYFD | | YVL | 2020 |
| | QRPSGVPDRFSGSKSGTSASLAISGLRS | | | | | | Y | | | (https://science.science |
| | EDEADYYCAAWDDSLIYVLFGGGTKLT | | | | | | | | | mag.org/content/early/ |
| | VL | | | | | | | | | 2020/06/19/science.abc |
| | | | | | | | | | | 6952/tab-pdf) |
| 2B04 | QAVVTQESALTTSPGETVTLTCRSSTG | IGHV2-9- | IGHJ4 | IGLV1 | IGLJ1 | 2087 | ARKDYYGRYY | 3296 | ALWYNNH | Wafaa Alsoussi et al., |
| | AVTTSNYANWVQEKPDHLFTGLIGGT | 1 | (Mouse) | (Mouse) | (Mouse) | | GMDY | | WV | 2020 |
| | NNRAPGVPARFSGSLIGDKAALTITGA | (Mouse) | | | | | | | | (https://www.jimmunol |
| | QTEDEAIYFCALWYNNHWVFGGGTK | | | | | | | | | .org/content/early/202 |
| | LTVL | | | | | | | | | 0/06/23/jimmunol.2000 |
| | | | | | | | | | | 583) |
| 2C02 | QIVLTQSPAIMSASPGEKVTMTCSASS | IGHV1-74 | IGHJ1 | IGKV4-55 | IGKJ5 | 2088 | AILDSYWYFD | 3297 | QQWSSYP | Wafaa Alsoussi et al., |
| | SVSYMYWYQQKPGSSPRLLIYDTSNLA | (Mouse) | (Mouse) | (Mouse) | (Mouse) | | V | | LT | 2020 |
| | SGVPIRFSGSGSGTSYSLTISRMEAEDA | | | | | | | | | (https://www.jimmunol |
| | ATYYCQQWSSYPLTFGAGTKLELK | | | | | | | | | org/content/early/202 |
| | | | | | | | | | | 0/06/23/jimmunol.2000 |
| | | | | | | | | | | 583) |
| 2C03 | DVVMTQTPLSLPVSLGDQASISCRSSQ | IGHV1-82 | IGHJ1 | IGKV1-117 | IGKJ2 | 2089 | ARKSYGYWH | 3298 | FQGSHVPY | Wafaa Alsoussi et al., |
| | SIVYSNGNTYLEWYLQKPGQSPKLLIYK | (Mouse) | (Mouse) | (Mouse) | (Mouse) | | DV | | T | 2020 |
| | VSNRFSGVPDRFSGSGSGTDFTLKISG | | | | | | | | | (https://www.jimmunol |
| | VEAEDLGVYYCFQGSHVPYTFGGGTK | | | | | | | | | org/content/early/202 |
| | LEIK | | | | | | | | | 0/06/23/jimmunol.2000 |
| | | | | | | | | | | 583) |
| 2C04 | DIVLTQSQASLAVSLGQRATISCRASES | IGHV1-26 | IGHJ2 | IGKV3-1 | IGKJ1 | 2090 | AAGKGDY | 3299 | QQSRKVP | Wafaa Alsoussi et al., |
| | VEYYGTSLMQWYQQKPGQPPKLLIYA | (Mouse) | (Mouse) | (Mouse) | (Mouse) | | | | WT | 2020 |
| | ASNVESGVPARFSGSGSGTDFSLNIHP | | | | | | | | | (https://www.jimmunol |
| | VEEDDIAMYFCQQSRKVPWTFGGGT | | | | | | | | | .org/content/early/202 |
| | KLEIK | | | | | | | | | 0/06/23/jimmunol.2000 |
| | | | | | | | | | | 583) |
| 2D01 | QIVLTQSPAIMSASPGEKVTMTCSASS | IGHV1-74 | IGHJ1 | IGKV4-55 | IGKJ5 | 2091 | AILDSYWYFD | 3300 | QQWSSYP | Wafaa Alsoussi et al., |
| | SVSYMYWYQQKPGSSPRLLIYDTSNLA | (Mouse) | (Mouse) | (Mouse) | (Mouse) | | V | | LT | 2020 |
| | SGVPIRFSGSGSGTSYSLTISRMEAEDA | | | | | | | | | (https://www.jimmunol |
| | ATYYCQQWSSYPLTFGAGTKLELK | | | | | | | | | .org/content/early/202 |
| | | | | | | | | | | 0/06/23/jimmunol.2000 |
| | | | | | | | | | | 583) |
| 2D08 | DIVLTQSQASLAVSLGQRATISCRASES | IGHV1-26 | IGHJ2 | IGKV3-1 | IGKJ1 | 2092 | AAGKGDY | 3301 | QQSRKVP | Wafaa Alsoussi et al., |
| | VEYYGTSLMQWYQQKPGQPPKLLIYA | (Mouse) | (Mouse) | (Mouse) | (Mouse) | | | | WT | 2020 |
| | ASNVESGVPARFSGSGSGTDFSLNIHP | | | | | | | | | (https://www.jimmunol |
| | VEEDDIAMYFCQQSRKVPWTFGGGT | | | | | | | | | .org/content/early/202 |
| | KLEIK | | | | | | | | | 0/06/23/jimmunol.2000 |
| | | | | | | | | | | 583) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| 2D11 | Ab | SARS-CoV2 | MERS- | | SARS-CoV2 | S; RBD | Immunised Mouse | 99 | EVQLQQSGPELVKPGASVKISCKASGYT | 1181 |
| | | | CoV, | | | | | | FTDYYMNWVKQSHGKSLEWIGDINPN | |
| | | | SARS- | | | | | | NGGTSYNQKFKGKATLTVDKSSSTAYME | |
| | | | CoV1 | | | | | | LRSLTSEDSAVYYCARKGDGYYGGFAYW | |
| | | | | | | | | | GQGTLVTVSA | |
| 2E06 | Ab | SARS-CoV1, | MERS- | SARS-CoV2 | | S; RBD | Immunised Mouse | 100 | EVQLQQSVADLVRPGASVKLSCTASGEN | 1182 |
| | | SARS-CoV2 | CoV | (weak) | | | | | IKNTYMHWVKQRPEQGLEWIGRIDPTN | |
| | | | | | | | | | GDTKYVSKFQGKATITADTSSNTAYLQLS | |
| | | | | | | | | | SLTSEDTAIYYCATYGSYYLYYYAMNYWG | |
| | | | | | | | | | QGTSVTVSS | |
| 2E10 | Ab | SARS-CoV1, | MERS- | | SARS-CoV2 | S; RBD | Immunised Mouse | 101 | EVQLQQPGAELVRPGASVKLSCKASGYT | 1183 |
| | | SARS-CoV2 | CoV | | | | | | FTDYYINWVKQRPGQGLEWIARIYPGT | |
| | | | | | | | | | GTTYYNEKFKGKATLTAEKSSSTAYMQLS | |
| | | | | | | | | | SLTSEDSAVYFCARYDGNLYYYAMDYW | |
| | | | | | | | | | GQGTSVTVSS | |
| 2F04 | Ab | SARS-CoV2 | MERS- | | SARS-CoV2 | S; RBD | Immunised Mouse | 102 | EVQLQQPGAELVKPGASVKMSCKASGY | 1184 |
| | | | CoV, | | | | | | TFTSYWITWVKQRPGQGLEWIGDIYPG | |
| | | | SARS- | | | | | | SGSTKYNEKFRSEATLTVDTSSTTAYMQL | |
| | | | CoV1 | | | | | | SSLTSEDSAVYYCARWDFYGSRTFDYWG | |
| | | | | | | | | | QGTTLTVSS | |
| 2H04 | Ab | SARS-CoV2 | MERS- | SARS-CoV2 | | S; RBD | Immunised Mouse | 103 | EVQLQQSGAELVKPGASVKMSCKASGY | 1185 |
| | | | CoV, | | | | | | TFTSYWITWVKQRPGQGLEWIGDIYPG | |
| | | | SARS- | | | | | | SGSTKYNEKFRSEATLTVDTSSTTAYMQL | |
| | | | CoV1 | | | | | | SSLTSEDSAVYYCARWDFYGSRTFDYWG | |
| | | | | | | | | | QGTTLTVSS | |
| 2M- | Ab | SARS-CoV2 | | | SARS-CoV2 | S; RBD | B-cells; SARS-CoV2 | 104 | EVQLVESGGGLVQPGGSLRLSCAASGFT | 1186 |
| 10B11 | | | | | | | Human Patient | | VSSNYMSWVRQAPGKGLEWVSVIYSG | |
| | | | | | | | | | GSTYYADSVKGRFTISRDNSKNTLYLQM | |
| | | | | | | | | | NSLRAEDTAVYYCARATWLRGVMDVW | |
| | | | | | | | | | GQGTTVTVSS | |
| 2M-12D7 | Ab | SARS-CoV2 | | | SARS-CoV2 | S; S2 | B-cells; SARS-CoV2 | 105 | EVQLVESGGGLVQPGRSLRLSCAASGFT | 1187 |
| | | | | | | | Human Patient | | FDDYVMHWVRQAPGKGLEWVSGINW | |
| | | | | | | | | | NSGSIGYADSVKGRFTISRDNAKNSLYLQ | |
| | | | | | | | | | MNSLRAEDTALYYCAKDVRYCSSTSCYFS | |
| | | | | | | | | | AFDIWGQGTMVTVSS | |
| 2M-13A3 | Ab | SARS-CoV2 | | | SARS-CoV2 | S; S2 | B-cells; SARS-CoV2 | 106 | EVQLVESGGGVVQPGRSLRLSCAASGFT | 1188 |
| | | | | | | | Human Patient | | FSGYAMHWVRQAPGKGLEWVAVISYD | |
| | | | | | | | | | GSNKYYADSVKGRFTISRDNSKNTLYLQ | |
| | | | | | | | | | MNSLRAEDTAVYYCARGGGSYYYWFDP | |
| | | | | | | | | | WGQGTMVTVSS | |
| 2M- | Ab | SARS-CoV2 | | | SARS-CoV2 | S; S2 | B-cells; SARS-CoV2 | 107 | EVQLVQWGAGLLKPSETLSLTCAVYGGS | 1189 |
| 13D11 | | | | | | | Human Patient | | FSGYYWSWIRQPPGKGLEWIGEINHSG | |
| | | | | | | | | | STNYNPSLKSRVTISVDTSKNQFSLKLSSV | |
| | | | | | | | | | TAADTAVYYCARAGYSSSWYGVRGVDP | |
| | | | | | | | | | WGQGTMVTVSS | |
| 2M-14B2 | Ab | SARS-CoV2 | | | SARS-CoV2 | S; S2 | B-cells; SARS-CoV2 | 108 | QVQLLQSGGGVVQPGRSLRLSCAASGF | 1190 |
| | | | | | | | Human Patient | | TFSTYGMHWVRQAPGKGLEWVAVISY | |
| | | | | | | | | | DGSNKYYADSVKGRFTISRDNSKNTLYL | |
| | | | | | | | | | QMNSLRAEDTAVYYCAKGSDIVVVPVG | |
| | | | | | | | | | NWFDPWGQGTLVTVSS | |
| 2M-14E4 | Ab | SARS-CoV2 | | | SARS-CoV2 | S; S2 | B-cells; SARS-CoV2 | 109 | QVQLQESGPGLVKPSETLSLACTVSGGS | 1191 |
| | | | | | | | Human Patient | | VSSDNSYWSWIRQPPGKGLEWIGYTFH | |
| | | | | | | | | | SGSANYNPSLKSRVTISVDTSKNQFSLKL | |
| | | | | | | | | | SSVTAADTAVYYCARVQRYYPDSSGFYG | |
| | | | | | | | | | RRFDIWGQGTLVTVSS | |
| 2M-14E5 | Ab | SARS-CoV2 | | | SARS-CoV2 | S; S2 | B-cells; SARS-CoV2 | 110 | EVQLVESGGGVVQPGRSLRLSCAASGFT | 1192 |
| | | | | | | | Human Patient | | FSTYSMHWVRQAPGKGLEWVAVISYD | |
| | | | | | | | | | GSNKYYADSVKGRFTISRDNSKNTLYLQ | |
| | | | | | | | | | VNSLRAEDTAVYYCARSGGGSYRGPFDY | |
| | | | | | | | | | WGQGTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| 2D11 | DIVLTQSPASLAVSLGQRATISCRASES | IGHV1-26 | IGHJ3 | IGKV3-2 | IGKJ1 | 2093 | ARKGDGYYG | 3302 | QQSKEVP | Wafaa Alsoussi et al., |
| | VDNYGISFMNWFQQKPGQPPKLLIYA | (Mouse) | (Mouse) | (Mouse) | (Mouse) | | GFAY | | WT | 2020 |
| | ASNQGSGVPARFSGSGSGTDFSLNIHP | | | | | | | | | (https://www.jimmunol |
| | MEEDDTAMYFFQQSKEVPWTFGGGT | | | | | | | | | .org/content/early/202 |
| | KLEIK | | | | | | | | | 0/06/23/jimmunol.2000 |
| | | | | | | | | | | 583) |
| 2E06 | DIVLTQSPASLTVSLGQRATISCKASQS | IGHV14-3 | IGHJ4 | IGKV3-4 | IGKJ2 | 2094 | ATYGSYYLY | 3303 | QQSNEDP | Wafaa Alsoussi et al., |
| | VDYGGDRYMNWYQQKPGQPPKLLIF | (Mouse) | (Mouse) | (Mouse) | (Mouse) | | YYAMNY | | YT | 2020 |
| | AASNLESGIPARFSGSGSGTDFTLNIHP | | | | | | | | | (https://www.jimmunol |
| | VEEEDAATYYCQQSNEDPYTFGGGTK | | | | | | | | | .org/content/early/202 |
| | LEIK | | | | | | | | | 0/06/23/jimmunol.2000 |
| | | | | | | | | | | 583) |
| 2E10 | DIVLTQSPASLAVSLGQRATMSCRASE | IGHV1-76 | IGHJ4 | IGKV3-4 | IGKJ5 | 2095 | ARYDGNLYYY | 3304 | QQNNEDP | Wafaa Alsoussi et al., |
| | SVDSYGNGFMHWYQQKPGQPPKLLI | (Mouse) | (Mouse) | (Mouse) | (Mouse) | | AMDY | | LT | 2020 |
| | YLASNLESGVPARFSGSGSRTDFSLTID | | | | | | | | | (https://www.jimmunol |
| | PVEADDAAIYYCQQNNEDPLTFGAGT | | | | | | | | | .org/content/early/202 |
| | KLELK | | | | | | | | | 0/06/23/jimmunol.2000 |
| | | | | | | | | | | 583) |
| 2F04 | DIVLTQSPAILSVSPGERVSFSCRASQN | IGHV1-55 | IGHJ2 | IGKV5-48 | IGKJ5 | 2096 | ARWDFYGSR | 3305 | QQSSSWP | Wafaa Alsoussi et al., |
| | IGTIIHWYQQRTNGSPRLLIKYASESVS | (Mouse) | (Mouse) | (Mouse) | (Mouse) | | TEDY | | LT | 2020 |
| | GIPSRFSGSGSGTDFTLSINSVESEDIA | | | | | | | | | (https://www.jimmunol |
| | DYYCQQSSSWPLTFGAGTKLELK | | | | | | | | | .org/content/early/202 |
| | | | | | | | | | | 0/06/23/jimmunol.2000 |
| | | | | | | | | | | 583) |
| 2H04 | DIVLTQSPAILSVSPGERVSFSCRASQN | IGKV5-48 | IGKJ5 | IGHV1-55 | IGHJ2 | 2097 | ARWDFYGSR | 3306 | QQSSSWP | Wafaa Alsoussi et al., |
| | IGTIIHWYQQRTNGSPRLLIKYASESVS | (Mouse) | (Mouse) | (Mouse) | (Mouse) | | TEDY | | LT | 2020 |
| | GIPSRFSGSGSGTDFTLSINSVESEDIA | | | | | | | | | (https://www.jimmunol |
| | DYYCQQSSSWPLTFGAGTKLELK | | | | | | | | | .org/content/early/202 |
| | | | | | | | | | | 0/06/23/jimmunol.2000 |
| | | | | | | | | | | 583) |
| 2M- | QSALTQPHSVSESPGKTVTISCTGSSGS | IGHV3-66 | IGHJ6 | IGLV6-57 | IGLJ3 | 2098 | ARATWLRGV | 3307 | QSYDSSNH | Xiangyang Chi et al., |
| 10B11 | IASNYVQWYQQRPGSAPTTVIYEDNQ | (Human) | (Human) | (Human) | (Human) | | MDVW | | WV | 2020 |
| | RPSGVPDRFSGSIDSSSNSASLTISGLKT | | | | | | | | | (https://science.science |
| | EDEADYYCQSYDSSNHWVFGGGTKLT | | | | | | | | | mag.org/content/early/ |
| | VL | | | | | | | | | 2020/06/19/science.abc |
| | | | | | | | | | | 6952/tab-pdf) |
| 2M-12D7 | AIRMTQSPSSLSASVGDRVTITCRASQ | IGHV3-9 | IGHJ3 | IGKV1-39 | IGKJ3 | 2099 | AKDVRYCSST | 3308 | QQSYSTPR | Xiangyang Chi et al., |
| | SITGYLNWYQQKPGKAPKLLISAASSL | (Human) | (Human) | (Human) | (Human) | | SCYFSAFDI | | T | 2020 |
| | QSGVPSRFSGSGSGTDFTLTISSLQPED | | | | | | | | | (https://science.science |
| | FATYYCQQSYSTPRTFGPGTKVEIK | | | | | | | | | mag.org/content/early/ |
| | | | | | | | | | | 2020/06/19/science.abc |
| | | | | | | | | | | 6952/tab-pdf) |
| 2M-13A3 | AIRMTQSPDSLAVSLGERATINCKSSQ | IGHV3-30 | IGHJ3 | IGKV4-1 | IGKJ5 | 2100 | ARGGGSYYY | 3309 | QQYYSTPF | Xiangyang Chi et al., |
| | SVLYSPNNKNYLAWYQQKPGQPPKLL | (Human) | (Human) | (Human) | (Human) | | WFDP | | T | 2020 |
| | IYWASTRESGVPDRFSGSGSGTDFTLTI | | | | | | | | | (https://science.science |
| | SSLQAEDVAVYYCQQYYSTPFTFGQGT | | | | | | | | | mag.org/content/early/ |
| | RLEIK | | | | | | | | | 2020/06/19/science.abc |
| | | | | | | | | | | 6952/tab-pdf) |
| 2M- | EIVMTQSPGTLSLSPGERATLSCRASQ | IGHV4-34 | IGHJ3 | IGKV3-20 | IGKJ1 | 2101 | ARAGYSSSWY | 3310 | QQYGSSRS | Xiangyang Chi et al., |
| 13D11 | SVSSSYLAWYQQKPGQAPRLLIYGASS | (Human) | (Human) | (Human) | (Human) | | GVRGVDP | | WT | 2020 |
| | RATGIPDRFSGSGSGTDFTLTISRLEPE | | | | | | | | | (https://science.science |
| | DFAVYYCQQYGSSRSWTFGQGTKVEI | | | | | | | | | mag.org/content/early/ |
| | K | | | | | | | | | 2020/06/19/science.abc |
| | | | | | | | | | | 6952/tab-pdf) |
| 2M-14B2 | AIRMTQSPSSLSASVGDRVTITCRASQ | IGHV3-30 | IGHJ5 | IGKV1-39 | IGKJ3 | 2102 | AKGSDIVVVP | 3311 | QQSYSTFT | Xiangyang Chi et al., |
| | SISSYLNWYQQKPGKAPKLLIYAASSLQ | (Human) | (Human) | (Human) | (Human) | | VGNWFDP | | LYT | 2020 |
| | SGVPSRFSGSGSGTDFTLTISSLQPEDF | | | | | | | | | (https://science.science |
| | ATYYCQQSYSTFTLYTFGQGTKVDIK | | | | | | | | | mag.org/content/early/ |
| | | | | | | | | | | 2020/06/19/science.abc |
| | | | | | | | | | | 6952/tab-pdf) |
| 2M-14E4 | DIVMTQSPSSLSASVGDRVSITCRASQ | IGHV4-61 | IGHJ4 | IGKV1-39 | IGKJ3 | 2103 | ARVQRYYPDS | 3312 | QQSHSFPF | Xiangyang Chi et al., |
| | NISNYLNWYQQKPGEAPKLLISAASSL | (Human) | (Human) | (Human) | (Human) | | SGFYGRRFDI | | T | 2020 |
| | QSGVPSRFGGSGSGTGFTLTINSLQPE | | | | | | | | | (https://science.science |
| | DFATYYCQQSHSFPFTFGPGTKVDIK | | | | | | | | | mag.org/content/early/ |
| | | | | | | | | | | 2020/06/19/science.abc |
| | | | | | | | | | | 6952/tab-pdf) |
| 2M-14E5 | AIRMTQSPSFLSVSVGDRVTITCRASQ | IGHV3-30 | IGHJ4 | IGKV1-9 | IGKJ4 | 2104 | ARSGGGSYR | 3313 | QQLNSYVT | Xiangyang Chi et al., |
| | GISSYLAWYQQKPGKAPKLLIYAASTL | (Human) | (Human) | (Human) | (Human) | | GPFDY | | | 2020 |
| | QSGVPSRFSGSGSGTEFTLTISSLQPED | | | | | | | | | (https://science.science |
| | FATYYCQQLNSYVTFGGGTKVEIK | | | | | | | | | mag.org/content/early/ |
| | | | | | | | | | | 2020/06/19/science.abc |
| | | | | | | | | | | 6952/tab-pdf) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| 2M-2D1 | Ab | SARS-CoV2 | | | SARS-CoV2 | S; S2 | B-cells; SARS-CoV2 | 111 | EVQLVESGPGLVKPSETLSLTCTVSGGSI | 1193 |
| | | | | | | | Human Patient | | SSSSYYWGWIRQPPGKGLEWIGSIYYSG | |
| | | | | | | | | | STYYNPSLKSRVTISVDTSKNQFSLKLSSV | |
| | | | | | | | | | TAADTAVYYSARGDRIQLWLLDAFDIW | |
| | | | | | | | | | GQGTMVTVSS | |
| 2M-2D4 | Ab | SARS-CoV2 | | | SARS-CoV2 | S; S2 | B-cells; SARS-CoV2 | 112 | EVQLVESGGGLVQPGGSLRLSCAASGFT | 1194 |
| | | | | | | | Human Patient | | FSSYAMSWVRQAPGKGLEWVSAISGSG | |
| | | | | | | | | | GSTYYADSVKGRFTISRDNSKNTLYLQM | |
| | | | | | | | | | NSLRAEDTAVYYCAKIGLGLGGLLRRYFD | |
| | | | | | | | | | YWGQGTLVTVSS | |
| 2M-2G12 | Ab | SARS-CoV2 | | | SARS-CoV2 | S; S2 | B-cells; SARS-CoV2 | 113 | QVQLVESGGGLLKPGETLSLSCAASGFTF | 1195 |
| | | | | | | | Human Patient | | SDYYWSWIRQAPGKGLEWIAVINHSGS | |
| | | | | | | | | | TIYYPYPVKGVFIFSRDTANNFSLKLMN | |
| | | | | | | | | | MMTSDTAVYYCGTRIMITWYSRRGMD | |
| | | | | | | | | | GWGKGVTVTVSS | |
| 2M-4G4 | Ab | SARS-CoV2 | | | SARS-CoV2 | S; RBD | B-cells; SARS-CoV2 | 114 | EVQLVESGAEVKKPGASVKVSCKASGYT | 1196 |
| | | | | | | | Human Patient | | FTSYYIHWVRQAPGQGLEWMGVINPS | |
| | | | | | | | | | GGSTTYAQKFQGRVTVTRDTSTSTVYM | |
| | | | | | | | | | ELSSLRSEDTAVYYCARERGDSSGYYEIIT | |
| | | | | | | | | | TANRRFGMDVWGQGTTVTVSS | |
| 2M-7E9 | Ab | SARS-CoV2 | | | SARS-CoV2 | S; S2 | B-cells; SARS-CoV2 | 115 | EVQLVESGAEVKKPGSSVKVSCKASGGT | 1197 |
| | | | | | | | Human Patient | | FSSFAISWVRQAPGQGLEWMGGIIPIFD | |
| | | | | | | | | | TTNYAQKFQGRVTITADESTRTAYMELS | |
| | | | | | | | | | SLRSEDTAVYYCARIPGWDRGTDRNWN | |
| | | | | | | | | | DDWGQGTTVTVSS | |
| 2M-8E7 | Ab | SARS-CoV2 | | | SARS-CoV2 | S; S2 | B-cells; SARS-CoV2 | 116 | EVQLVESGAEVKKPGSSVKVSCKASGGT | 1198 |
| | | | | | | | Human Patient | | FSSYAISWVRQAPGQGLEWMGGIIPIFG | |
| | | | | | | | | | TANYAQKFQGRVTVTADESTSTAYMELS | |
| | | | | | | | | | SLRSEDTAVYYCARTYSFDSSGYYYDYW | |
| | | | | | | | | | GQGTMVTVSS | |
| 2M-8H10 | Ab | SARS-CoV2 | | | SARS-CoV2 | S; S2 | B-cells; SARS-CoV2 | 117 | QVQLVESGGGVVLPGRSLRLSCAASGFT | 1199 |
| | | | | | | | Human Patient | | FSTFAMHWVRQAPGKGLEWVAVISDE | |
| | | | | | | | | | GSNKYYADSVKGRFTISRDNSRNTLYLQ | |
| | | | | | | | | | MNSLRAEDTAVYYCARAFYDSNWSVGS | |
| | | | | | | | | | YFDSWGQGTPVTVSS | |
| 2M-9F10 | Ab | SARS-CoV2 | | | SARS-CoV2 | S; S2 | B-cells; SARS-CoV2 | 118 | EVQLLQSGGDLIQPGRSLRLSCAASGFSF | 1200 |
| | | | | | | | Human Patient | | EDYAMHWVRQAPGQGLEWVSGISYN | |
| | | | | | | | | | GGSIDYVDSVKGRFTISRDNAKNALYLE | |
| | | | | | | | | | MNSLRPEDTAFYYCAKDSVRREYTHARV | |
| | | | | | | | | | PFDNWGLGTLVTVSS | |
| 2M-9H1 | Ab | SARS-CoV2 | | | SARS-CoV2 | S; S2 | B-cells; SARS-CoV2 | 119 | EVQLLESGGGVVQPGRSLRLSCVASGEN | 1201 |
| | | | | | | | Human Patient | | FNNYGMHWVRQAPGKGLEWLAALSYE | |
| | | | | | | | | | GSKEHYADSLKGRFTVSRDYSRATLHLH | |
| | | | | | | | | | MNSLEPEDTAVYFCAKSSKIFYLGESREV | |
| | | | | | | | | | DYWGRGTLVTVSS | |
| 31B5 | Ab | SARS-CoV2 | | SARS-CoV2 | | S; RBD | B-cells; SARS-CoV2 | | ND | |
| | | | | | | | Human Patient | | | |
| 31B9 | Ab | SARS-CoV2 | | | SARS-CoV2 | S; RBD | B-cells; SARS-CoV2 | | ND | |
| | | | | | | | Human Patient | | | |
| 32D4 | Ab | SARS-CoV2 | | SARS-CoV2 | | S; RBD | B-cells; SARS-CoV2 | | ND | |
| | | | | | | | Human Patient | | | |
| 413-2 | Ab | SARS-CoV2 | SARS- | SARS-CoV2 | SARS-CoV1 | S; non- | B-cells; SARS-CoV2 | | ND | |
| | | | CoV1 | | | RBD | Human Patient | | | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| 2M-2D1 | QSVLTQLPSASGTPGQTVTISCSGNVF | IGHV4-39 | IGHJ3 | IGLV1-47 | IGLJ3 | 2105 | ARGDRIQLW | 3314 | AARDDSLS | Xiangyang Chi et al., |
| | KEKSNYVYWYQQLPGTAPKLLIYRNN | (Human) | (Human) | (Human) | (Human) | | LLDAFDI | | GWV | 2020 |
| | HRPSGVPDRFSGSKSGTSPSLAISGLRS | | | | | | | | | (https://science.science |
| | EDEADYYCAARDDSLSGWVFSGRTKL | | | | | | | | | mag.org/content/early/ |
| | TVL | | | | | | | | | 2020/06/19/science.abc |
| | | | | | | | | | | 6952/tab-pdf) |
| 2M-2D4 | EIVMTQSPATLSLSPGERATLSCRASQS | IGHV3-23 | IGHJ4 | IGKV3-11 | IGKJ3 | 2106 | AKIGLGLG | 3315 | QQRTNWP | Xiangyang Chi et al., |
| | VSTSLAWYQQKPGQAPRLLIYDASNR | (Human) | (Human) | (Human) | (Human) | | GLLRRYFDY | | L | 2020 |
| | ATGVPARFSGSGSGTDFTLTISSLEPDD | | | | | | | | | (https://science.science |
| | FAVYYCQQRTNWPLFGPGTKVEIK | | | | | | | | | mag.org/content/early/ |
| | | | | | | | | | | 2020/06/19/science.abc |
| | | | | | | | | | | 6952/tab-pdf) |
| 2M-2G12 | DIQMTQSPSSLSVSAGERATLSCRASQ | IGHV3-11 | IGHJ6 | IGKV1-39 | IGKJ1 | 2107 | GTRIMITW | 3316 | QQNYSTW | Xiangyang Chi et al., |
| | SVSSYLIWCHQKPGKAPKLLIYDASTLQ | (Human) | (Human) | (Human) | (Human) | | YSRRGMDG | | T | 2020 |
| | TGVPSRFIGGGSGTNFTLTIISLQPEDF | | | | | | | | | (https://science.science |
| | AAYYCQQNYSTWTIGQGSRVEMK | | | | | | | | | mag.org/content/early/ |
| | | | | | | | | | | 2020/06/19/science.abc |
| | | | | | | | | | | 6952/tab-pdf) |
| 2M-4G4 | QSVLTQPASVSGSPGQSITISCTGTSSD | IGHV1-46 | IGHJ6 | IGLV2-23 | IGLJ3 | 2108 | ARERGDSS | 3317 | CSYAVSST | Xiangyang Chi et al., |
| | VGMYNLVSWYQQHPGQAPKLMIYE | (Human) | (Human) | (Human) | (Human) | | GYYEIITT | | WV | 2020 |
| | GSKRPSGVSNRFSGSKSGNTASLTISGL | | | | | | ANRRFGMDV | | | (https://science.science |
| | QAEDEAYYYCCSYAVSSTWVFGGGTK | | | | | | | | | mag.org/content/early/ |
| | LTVL | | | | | | | | | 2020/06/19/science.abc |
| | | | | | | | | | | 6952/tab-pdf) |
| 2M-7E9 | DIVMTQSPATLSLSPGERATLSCRASQ | IGHV1-69 | IGHJ6 | IGKV3-11 | IGKJ3 | 2109 | ARIPGWDRG | 3318 | QQRSNWP | Xiangyang Chi et al., |
| | SVSNFLAWYQQKPGQAPRLLIYDASN | (Human) | (Human) | (Human) | (Human) | | TDRNWNDD | | PAFT | 2020 |
| | RATGIPARFSGSGSGTDFTLTISSLEPE | | | | | | | | | (https://science.science |
| | DFAVYYCQQRSNWPPAFTFGPGTKVE | | | | | | | | | mag.org/content/early/ |
| | IK | | | | | | | | | 2020/06/19/science.abc |
| | | | | | | | | | | 6952/tab-pdf) |
| 2M-8E7 | EIVLTQSPATLSLSPGERATLSCRASQS | IGHV1-69 | IGHJ4 | IGKV3-11 | IGKJ5 | 2110 | ARTYSFDS | 3319 | QQRSNWP | Xiangyang Chi et al., |
| | VSSYLAWYQQKPGQAPRLLIYDASNR | (Human) | (Human) | (Human) | (Human) | | SGYYYDY | | PKIT | 2020 |
| | ATGIPARFSGSGSGTDFTLTISSLEPEDF | | | | | | | | | (https://science.science |
| | AVYYCQQRSNWPPKITFGQGTRLEIK | | | | | | | | | mag.org/content/early/ |
| | | | | | | | | | | 2020/06/19/science.abc |
| | | | | | | | | | | 6952/tab-pdf) |
| 2M-8H10 | DIQMTQSPDSLAVSLGERATIKCKSSQ | IGHV3-30 | IGHJ5 | IGKV4-1 | IGKJ1 | 2111 | ARAFYDSNW | 3320 | QQYYNNQ | Xiangyang Chi et al., |
| | SVLHSSNNKNYLAWYQQKAGQPPSLL | (Human) | (Human) | (Human) | (Human) | | SVGSYFDS | | WT | 2020 |
| | LYWASTRESGVPDRFSGSGSGTDFTLT | | | | | | | | | (https://science.science |
| | ISSLQAEDVAVYYCQQYYNNQWTFGQ | | | | | | | | | mag.org/content/early/ |
| | GTKVDIK | | | | | | | | | 2020/06/19/science.abc |
| | | | | | | | | | | 6952/tab-pdf) |
| 2M-9F10 | AIRMTQSPSSLSASVGDRVTISCRATQ | IGHV3-9 | IGHJ4 | IGKV1-39 | IGKJ3 | 2112 | AKDSVRRE | 3321 | QQSFVSP | Xiangyang Chi et al., |
| | NINYFLNWYQQKPGRAPKLLIYAASGL | (Human) | (Human) | (Human) | (Human) | | YTHARVPF | | RT | 2020 |
| | QSGVPSRFSGSGTGRVFTLTINSLQPD | | | | | | DN | | | (https://science.science |
| | DFATYYCQQSFVSPRTFGQGTKVDIK | | | | | | | | | mag.org/content/early/ |
| | | | | | | | | | | 2020/06/19/science.abc |
| | | | | | | | | | | 6952/tab-pdf) |
| 2M-9H1 | DIVMTQSPSSLSAFVGDRVTITCRASR | IGHV3-30 | IGHJ4 | IGKV1-17 | IGKJ4 | 2113 | AKSSKIFYL | 3322 | LQHKSYP | Xiangyang Chi et al., |
| | DIGGDLSWFQQKPGKAPERLIYAASSL | (Human) | (Human) | (Human) | (Human) | | GESREVDY | | LT | 2020 |
| | ESGVPSRFSGSGSATEFALTITSLQPED | | | | | | | | | (https://science.science |
| | FATYYCLQHKSYPLTFGGGTKVDIK | | | | | | | | | mag.org/content/early/ |
| | | | | | | | | | | 2020/06/19/science.abc |
| | | | | | | | | | | 6952/tab-pdf) |
| 31B5 | ND | ND | ND | ND | ND | 2114 | ARVEYYYG | | ND | Xiangyu Chen et al., |
| | | | | | | | SGSYMPWYF | | | 2020 |
| | | | | | | | DL | | | (https://www.nature.co |
| | | | | | | | | | | m/articles/s41423-020- |
| | | | | | | | | | | 0426-7) |
| 31B9 | ND | ND | ND | ND | ND | 2115 | ATYYYDSS | | ND | Xiangyu Chen et al., |
| | | | | | | | GYSYGMDV | | | 2020 |
| | | | | | | | | | | (https://www.nature.co |
| | | | | | | | | | | m/articles/s41423-020- |
| | | | | | | | | | | 0426-7) |
| 32D4 | ND | ND | ND | ND | ND | 2116 | TEEGSGSE | | ND | Xiangyu Chen et al., |
| | | | | | | | GPIEFDY | | | 2020 |
| | | | | | | | | | | (https://www.nature.co |
| | | | | | | | | | | m/articles/s41423-020- |
| | | | | | | | | | | 0426-7) |
| 413-2 | ND | ND | ND | ND | ND | 2117 | ARDNNYRN | 3323 | QQYGSSP | Jinkai Wan et al., 2020 |
| | | | | | | | YYYYMDVW | | PLT | (https://www.sciencedi |
| | | | | | | | | | | rect.com/science/articl |
| | | | | | | | | | | e/pii/S22111247203089 |
| | | | | | | | | | | 98) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| 414-1 | Ab | SARS-CoV2 | SARS- | SARS-CoV2 | SARS-CoV1 | S; RBD | B-cells; SARS-CoV2 | | ND | |
| | | | CoV1 | | | | Human Patient | | | |
| 47D11 | Ab | SARS-CoV1, | | SARS-CoV2 | | S; RBD | B-cells; SARS-CoV1 | | ND | |
| | | SARS-CoV2 | | and SARS- | | | Human Patient | | | |
| | | | | CoV1 | | | | | | |
| 4A8 | Ab | SARS-CoV2 | | SARS-CoV2 | | S; S1 (non- | B-cells; SARS-CoV2 | 120 | EVQLVESGAEVKKPGASVKVSCKVSGYT | 1202 |
| | | | | | | RBD) | Human Patient | | LTELSMHWVRQAPGKGLEWMGGFDP | |
| | | | | | | | | | EDGETMYAQKFQGRVTMTEDTSTDTAY | |
| | | | | | | | | | MELSSLRSEDTAVYYCATSTAVAGTPDLF | |
| | | | | | | | | | DYYYGMDVWGQGTTVTVSS | |
| 505-3 | Ab | SARS-CoV2 | SARS- | SARS-CoV2 | SARS-CoV1 | S; RBD | B-cells; SARS-CoV2 | | ND | |
| | | | CoV1 | | | | Human Patient | | | |
| 505-5 | Ab | SARS-CoV2 | SARS- | SARS-CoV2 | SARS-CoV1 | S; RBD | B-cells; SARS-CoV2 | | ND | |
| | | | CoV1 | | | | Human Patient | | | |
| 505-8 | Ab | SARS-CoV2 | SARS- | SARS-CoV2 | SARS-CoV1 | S; non- | B-cells; SARS-CoV2 | | ND | |
| | | | CoV1 | | | RBD | Human Patient | | | |
| 515-1 | Ab | SARS-CoV2 | SARS- | SARS-CoV2 | SARS-CoV1 | S; RBD | B-cells; SARS-CoV2 | | ND | |
| | | | CoV1 | | | | Human Patient | | | |
| 515-5 | Ab | SARS-CoV2 | SARS- | SARS-CoV2 | SARS-CoV1 | S; non- | B-cells; SARS-CoV2 | | ND | |
| | | | CoV1 | | | RBD | Human Patient | | | |
| 553-15 | Ab | SARS-CoV1, | | SARS-CoV1, | | S; RBD | B-cells; SARS-CoV2 | | ND | |
| | | SARS-CoV2 | | SARS-CoV2 | | | Human Patient | | | |
| 553-49 | Ab | SARS-CoV2 | SARS- | SARS-CoV2 | SARS-CoV1 | S; RBD | B-cells; SARS-CoV2 | | ND | |
| | | | CoV1 | | | | Human Patient | | | |
| 553-60 | Ab | SARS-CoV2 | SARS- | SARS-CoV2 | SARS-CoV1 | S; RBD | B-cells; SARS-CoV2 | | ND | |
| | | | CoV1 | | | | Human Patient | | | |
| 553-63 | Ab | SARS-CoV2 | SARS- | SARS-CoV2 | SARS-CoV1 | S; RBD | B-cells; SARS-CoV2 | | ND | |
| | | | CoV1 | | | | Human Patient | | | |
| 8D2 | Ab | SARS-CoV2 | | | SARS-CoV2 | S; S2 | B-cells; SARS-CoV2 | 121 | EVQLVESGGGLVQPGGSLRLSCAASGFT | 1203 |
| | | | | | | | Human Patient | | FSSYWMSWVRQAPGKGLEWVANINQ | |
| | | | | | | | | | DGSEKYYVDSVKGRFTISRDNAKNSLYL | |
| | | | | | | | | | QVNSLRAEDTAVYYCARDWDYDILTGS | |
| | | | | | | | | | WFGAFDIWGQGTTVTVSS | |
| 8D9 | Ab | SARS-CoV2 | | | SARS-CoV2 | S; S2 | B-cells; SARS-CoV2 | 122 | EVQLLESGGGLVQPGGSLRLSCTASGFT | 1204 |
| | | | | | | | Human Patient | | FSSYWMSWVRQAPGKGLEWVANIQQ | |
| | | | | | | | | | DGSEKYYVDSVKGRFTISRDNAKNSLYL | |
| | | | | | | | | | QMNSLRAEDTAVYYCARPTIGYSYGSDY | |
| | | | | | | | | | WGQGTTVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| 414-1 | ND | ND | ND | ND | ND | 2118 | ARGALGCSST | 3324 | AAWDDSL | Jinkai Wan et al., 2020 |
| | | | | | | | SCYPNNFDY | | NGVV | (https://www.sciencedi |
| | | | | | | | W | | | rect.com/science/articl |
| | | | | | | | | | | e/pii/S22111247203089 |
| | | | | | | | | | | 98) |
| 47D11 | ND | ND | ND | ND | ND | 2119 | ND | | ND | Chunyan Wang et al., |
| | | | | | | | | | | 2020 |
| | | | | | | | | | | (https://www.nature.co |
| | | | | | | | | | | m/articles/s41467-020- |
| | | | | | | | | | | 16256-y) |
| 4A8 | EIVMTQSPLSSPVTLGQPASISCRSSQS | IGHV1-24 | IGHJ6 | IGKV2-24 | IGKJ3 | 2120 | ATSTAVAGTP | 3325 | TQATQFPY | Xiangyang Chi et al., |
| | LVHSDGNTYLSWLQQRPGQPPRLLIY | (Human) | (Human) | (Human) | (Human) | | DLFDYYYGM | | T | 2020 |
| | KISNRFSGVPDRFSGSGAGTDFTLKISR | | | | | | DV | | | (https://science.science |
| | VEAEDVGVYYCTQATQFPYTFGQGTK | | | | | | | | | mag.org/content/early/ |
| | VDIK | | | | | | | | | 2020/06/19/science.abc |
| | | | | | | | | | | 6952/tab-pdf) |
| 505-3 | ND | ND | ND | ND | ND | 2121 | ARDFISRPRG | 3326 | MQGTHW | Jinkai Wan et al., 2020 |
| | | | | | | | YRW | | PPT | (https://www.sciencedi |
| | | | | | | | | | | rect.com/science/articl |
| | | | | | | | | | | e/pii/S22111247203089 |
| | | | | | | | | | | 98) |
| 505-5 | ND | ND | ND | ND | ND | 2122 | ARDFISRPRG | 3327 | MQGTHW | Jinkai Wan et al., 2020 |
| | | | | | | | YRW | | PPT | (https://www.sciencedi |
| | | | | | | | | | | rect.com/science/articl |
| | | | | | | | | | | e/pii/S22111247203089 |
| | | | | | | | | | | 98) |
| 505-8 | ND | ND | ND | ND | ND | 2123 | ARGQDDFWS | 3328 | SSYTSSSIV | Jinkai Wan et al., 2020 |
| | | | | | | | MNWFDPW | | V | (https://www.sciencedi |
| | | | | | | | | | | rect.com/science/articl |
| | | | | | | | | | | e/pii/S22111247203089 |
| | | | | | | | | | | 98) |
| 515-1 | ND | ND | ND | ND | ND | 2124 | ARDFISRPRG | 3329 | MQGTHW | Jinkai Wan et al., 2020 |
| | | | | | | | YRW | | PPT | (https://www.sciencedi |
| | | | | | | | | | | rect.com/science/articl |
| | | | | | | | | | | e/pii/S22111247203089 |
| | | | | | | | | | | 98) |
| 515-5 | ND | ND | ND | ND | ND | 2125 | TRGSQWATI | 3330 | LQHNSYPI | Jinkai Wan et al., 2020 |
| | | | | | | | NDARFDYW | | T | (https://www.sciencedi |
| | | | | | | | | | | rect.com/science/articl |
| | | | | | | | | | | e/pii/S22111247203089 |
| | | | | | | | | | | 98) |
| 553-15 | ND | ND | ND | ND | ND | 2126 | ARVWYYYGP | 3331 | QSYDGSN | Jinkai Wan et al., 2020 |
| | | | | | | | RDYW | | HNVV | (https://www.sciencedi |
| | | | | | | | | | | rect.com/science/articl |
| | | | | | | | | | | e/pii/S22111247203089 |
| | | | | | | | | | | 98) |
| 553-49 | ND | ND | ND | ND | ND | 2127 | AKDSSSWYNY | 3332 | QSYDSSNH | Jinkai Wan et al., 2020 |
| | | | | | | | YGMDVW | | VV | (https://www.sciencedi |
| | | | | | | | | | | rect.com/science/articl |
| | | | | | | | | | | e/pii/S22111247203089 |
| | | | | | | | | | | 98) |
| 553-60 | ND | ND | ND | ND | ND | 2128 | ARVFRGSQY | 3333 | QQYGSSPP | Jinkai Wan et al., 2020 |
| | | | | | | | WFDPW | | T | (https://www.sciencedi |
| | | | | | | | | | | rect.com/science/articl |
| | | | | | | | | | | e/pii/S22111247203089 |
| | | | | | | | | | | 98) |
| 553-63 | ND | ND | ND | ND | ND | 2129 | ARMEAPKLTL | 3334 | QQSYSIPR | Jinkai Wan et al., 2020 |
| | | | | | | | DPW | | T | (https://www.sciencedi |
| | | | | | | | | | | rect.com/science/articl |
| | | | | | | | | | | e/pii/S22111247203089 |
| | | | | | | | | | | 98) |
| 8D2 | DIQMTQSPSSLSASVGDRVTITCRASQ | IGHV3-7 | IGHJ3 | IGKV1-17 | IGKJ4 | 2130 | ARDWDYDILT | 3335 | LQHNSYPL | Xiangyang Chi et al., |
| | GIRNDLGWYQQKPGKAPKRLIYAASSL | (Human) | (Human) | (Human) | (Human) | | GSWFGAFDI | | T | 2020 |
| | QSGVPSRFSGSGSGTEFTLTISSLQPED | | | | | | | | | (https://science.science |
| | FATYYCLQHNSYPLTFGGGTKVEIK | | | | | | | | | mag.org/content/early/ |
| | | | | | | | | | | 2020/06/19/science.abc |
| | | | | | | | | | | 6952/tab-pdf) |
| 8D9 | SYELTQPPSVSVSPGQTASITCSGDKLG | IGHV3-7 | IGHJ4 | IGLV3-1 | IGLJ3 | 2131 | ARPTIGYSYGS | 3336 | QAWDSST | Xiangyang Chi et al., |
| | DKYACWYRQKPGQSPVLVIYQDSKRP | (Human) | (Human) | (Human) | (Human) | | DY | | GV | 2020 |
| | SGIPERFSGSNSGNTATLTISGTQAMD | | | | | | | | | (https://science.science |
| | EADYYCQAWDSSTGVFGGGTKLTVL | | | | | | | | | mag.org/content/early/ |
| | | | | | | | | | | 2020/06/19/science.abc |
| | | | | | | | | | | 6952/tab-pdf) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| 9A1 | Ab | SARS-CoV2 | | | SARS-CoV2 | S; S2 | B-cells; SARS-CoV2 | 123 | EVQLLESGGGVVQPGRSLRLSCVVSGFT | 1205 |
| | | | | | | | Human Patient | | FNNYGMHWVRQAPGKGLEWVAVISYE | |
| | | | | | | | | | GSVKYYGDHVDGRFTISRDPFKNTLYLH | |
| | | | | | | | | | MNNLRPDDTAVYYCAKVSAIFWLGQGL | |
| | | | | | | | | | SPIDVWGQGTTVTVSS | |
| Ab_510A4 | Ab | SARS-CoV2 | | SARS-CoV2 | | S; RBD | B-cells; SARS-CoV2 | | ND | |
| | | | | (weak) | | | Human Patient | | | |
| Ab_510A5 | Ab | SARS-CoV2 | | | SARS-CoV2 | S; RBD | B-cells; SARS-CoV2 | | ND | |
| | | | | | | | Human Patient | | | |
| Ab_510D7 | Ab | SARS-CoV2 | | SARS-CoV2 | | S; RBD | B-cells; SARS-CoV2 | | ND | |
| | | | | | | | Human Patient | | | |
| Ab_510G4 | Ab | SARS-CoV2 | | SARS-CoV2 | | S; RBD | B-cells; SARS-CoV2 | | ND | |
| | | | | | | | Human Patient | | | |
| Ab_510H10 | Ab | SARS-CoV2 | | | | S; RBD | B-cells; SARS-CoV2 | | ND | |
| | | | | | | | Human Patient | | | |
| Ab_510H2 | Ab | SARS-CoV2 | | SARS-CoV2 | | S; RBD | B-cells; SARS-CoV2 | | ND | |
| | | | | (weak) | | | Human Patient | | | |
| Ab_510H4 | Ab | SARS-CoV2 | | SARS-CoV2 | | S; RBD | B-cells; SARS-CoV2 | | ND | |
| | | | | | | | Human Patient | | | |
| Ab_510H7 | Ab | SARS-CoV2 | | SARS-CoV2 | | S; RBD | B-cells; SARS-CoV2 | | ND | |
| | | | | | | | Human Patient | | | |
| Ab_511A1 | Ab | SARS-CoV2 | | | | S; RBD | B-cells; SARS-CoV2 | | ND | |
| | | | | | | | Human Patient | | | |
| Ab_511A5 | Ab | SARS-CoV2 | | SARS-CoV2 | | S; RBD | B-cells; SARS-CoV2 | | ND | |
| | | | | | | | Human Patient | | | |
| Ab_511B11 | Ab | SARS-CoV2 | | | | S; RBD | B-cells; SARS-CoV2 | | ND | |
| | | | | | | | Human Patient | | | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| 9A1 | DIVMTQSPATLSASPGERVTLSCRASQ | IGHV3-30 | IGHJ6 | IGKV3-15 | IGKJ3 | 2132 | AKVSAIFWLG | 3337 | HQYSKWP | Xiangyang Chi et al., |
| | NIRNNLAWYQQKPGQAPRLLIHGAST | (Human) | (Human) | (Human) | (Human) | | QGLSPIDV | | VT | 2020 |
| | RAAGAPARFSGSGSDTQFTLTVSSLQS | | | | | | | | | (https://science.science |
| | EDFAVYYCHQYSKWPVTFGGGTKVDI | | | | | | | | | mag.org/content/early/ |
| | K | | | | | | | | | 2020/06/19/science.abc |
| | | | | | | | | | | 6952/tab-pdf) |
| Ab_510A4 | ND | IGHV2-70 | IGHJ4 | IGKV2-28 | IGKJ1 | 2133 | ARVQVAAAG | 3338 | MQALQM | Xiaojian Han et al., 2020 |
| | | (Human) | (Human) | (Human) | (Human) | | SPYDY | | GT | (https://www.biorxiv.or |
| | | | | | | | | | | g/content/10.1101/202 |
| | | | | | | | | | | 0.08.19.253369v2.full.p |
| | | | | | | | | | | df+html) |
| Ab_510A5 | ND | IGHV3-9 | IGHJ4 | IGKV1-39 | IGKJ2 | 2134 | AKDRGYEILTP | 3339 | QQSYSTPP | Xiaojian Han et al., 2020 |
| | | (Human) | (Human) | (Human) | (Human) | | ASFDY | | YT | (https://www.biorxiv.or |
| | | | | | | | | | | g/content/10.1101/202 |
| | | | | | | | | | | 0.08.19.253369v2.full.p |
| | | | | | | | | | | df+html) |
| Ab_510D7 | ND | IGHV1-69 | IGHJ4 | IGKV3-20 | IGKJ2 | 2135 | ATGRYTYGYG | 3340 | QQYGSSRT | Xiaojian Han et al., 2020 |
| | | (Human) | (Human) | (Human) | (Human) | | YYFDY | | | (https://www.biorxiv.or |
| | | | | | | | | | | g/content/10.1101/202 |
| | | | | | | | | | | 0.08.19.253369v2.full.p |
| | | | | | | | | | | df+html) |
| Ab_510G4 | ND | IGHV4-31 | IGHJ4 | IGKV1-33 | IGKJ4 | 2136 | ARDYGGNSN | 3341 | QQYDTLPL | Xiaojian Han et al., 2020 |
| | | (Human) | (Human) | (Human) | (Human) | | YFHY | | T | (https://www.biorxiv.or |
| | | | | | | | | | | g/content/10.1101/202 |
| | | | | | | | | | | 0.08.19.253369v2.full.p |
| | | | | | | | | | | df+html) |
| Ab_510H10 | ND | IGHV2-70 | IGHJ4 | IGKV1-39 | IGKJ2 | 2137 | ARIQRGIAAD | 3342 | QQSYSTPR | Xiaojian Han et al., 2020 |
| | | (Human) | (Human) | (Human) | (Human) | | Y | | T | (https://www.biorxiv.or |
| | | | | | | | | | | g/content/10.1101/202 |
| | | | | | | | | | | 0.08.19.253369v2.full.p |
| | | | | | | | | | | df+html) |
| Ab_510H2 | ND | IGHV3-66 | IGHJ4 | IGKV1-9 | IGKJ4 | 2138 | ARDKWEGTF | 3343 | QQLNSYPR | Xiaojian Han et al., 2020 |
| | | (Human) | (Human) | (Human) | (Human) | | DY | | MT | (https://www.biorxiv.or |
| | | | | | | | | | | g/content/10.1101/202 |
| | | | | | | | | | | 0.08.19.253369v2.full.p |
| | | | | | | | | | | df+html) |
| Ab_510H4 | ND | IGHV3-66 | IGHJ6 | IGKV3-11 | IGKJ1 | 2139 | AETGWDGM | 3344 | QQRSNWP | Xiaojian Han et al., 2020 |
| | | (Human) | (Human) | (Human) | (Human) | | DV | | GT | (https://www.biorxiv.or |
| | | | | | | | | | | g/content/10.1101/202 |
| | | | | | | | | | | 0.08.19.253369v2.full.p |
| | | | | | | | | | | df+html) |
| Ab_510H7 | ND | IGHV4-59 | IGHJ5 | IGKV1D-13 | IGKJ4 | 2140 | ARHCPWQQL | 3345 | QQFNNFLL | Xiaojian Han et al., 2020 |
| | | (Human) | (Human) | (Human) | (Human) | | VSNWFDP | | T | (https://www.biorxiv.or |
| | | | | | | | | | | g/content/10.1101/202 |
| | | | | | | | | | | 0.08.19.253369v2.full.p |
| | | | | | | | | | | df+html) |
| Ab_511A1 | ND | IGHV4-31 | IGHJ6 | IGKV3-15 | IGKJ1 | 2141 | AREKIRSIAAA | 3346 | QQYNNWP | Xiaojian Han et al., 2020 |
| | | (Human) | (Human) | (Human) | (Human) | | GTVYYYGMD | | PWT | (https://www.biorxiv.or |
| | | | | | | | V | | | g/content/10.1101/202 |
| | | | | | | | | | | 0.08.19.253369v2.full.p |
| | | | | | | | | | | df+html) |
| Ab_511A5 | ND | IGHV4-31 | IGHJ2 | IGLV3-21 | IGLJ1 | 2142 | ARIYRGTMVV | 3347 | QVWDSSA | Xiaojian Han et al., 2020 |
| | | (Human) | (Human) | (Human) | (Human) | | VFSDLHWYFD | | DHYV | (https://www.biorxiv.or |
| | | | | | | | L | | | g/content/10.1101/202 |
| | | | | | | | | | | 0.08.19.253369v2.full.p |
| | | | | | | | | | | df+html) |
| Ab_511B11 | ND | IGHV3-7 | IGHJ4 | IGLV1-40 | IGLJ1 | 2143 | AGLFWYGGY | 3348 | QSYDRSLS | Xiaojian Han et al., 2020 |
| | | (Human) | (Human) | (Human) | (Human) | | FDY | | VLYV | (https://www.biorxiv.or |
| | | | | | | | | | | g/content/10.1101/202 |
| | | | | | | | | | | 0.08.19.253369v2.full.p |
| | | | | | | | | | | df+html) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| Ab_511B4 | Ab | SARS-CoV2 | | SARS-CoV2 | | S; RBD | B-cells; SARS-CoV2 | | ND | |
| | | | | | | | Human Patient | | | |
| Ab_511D11 | Ab | SARS-CoV2 | | | | S; RBD | B-cells; SARS-CoV2 | | ND | |
| | | | | | | | Human Patient | | | |
| Ab_511E5 | Ab | SARS-CoV2 | | | | S; RBD | B-cells; SARS-CoV2 | | ND | |
| | | | | | | | Human Patient | | | |
| Ab_511E7 | Ab | SARS-CoV2 | | | | S; RBD | B-cells; SARS-CoV2 | | ND | |
| | | | | | | | Human Patient | | | |
| Ab_511E9 | Ab | SARS-CoV2 | | | | S; RBD | B-cells; SARS-CoV2 | | ND | |
| | | | | | | | Human Patient | | | |
| Ab_511G5 | Ab | SARS-CoV2 | | | | S; RBD | B-cells; SARS-CoV2 | | ND | |
| | | | | | | | Human Patient | | | |
| Ab_511G7 | Ab | SARS-CoV2 | | SARS-CoV2 | | S; RBD | B-cells; SARS-CoV2 | | ND | |
| | | | | | | | Human Patient | | | |
| Ab_511H11 | Ab | SARS-CoV2 | | | | S; RBD | B-cells; SARS-CoV2 | | ND | |
| | | | | | | | Human Patient | | | |
| Ab_511H7 | Ab | SARS-CoV2 | | | | S; RBD | B-cells; SARS-CoV2 | | ND | |
| | | | | | | | Human Patient | | | |
| Ab_51A1 | Ab | SARS-CoV2 | | SARS-CoV2 | | S; RBD | B-cells; SARS-CoV2 | | ND | |
| | | | | (weak) | | | Human Patient | | | |
| Ab_51A3 | Ab | SARS-CoV2 | | | | S; RBD | B-cells; SARS-CoV2 | | ND | |
| | | | | | | | Human Patient | | | |
| Ab_51D2 | Ab | SARS-CoV2 | | SARS-CoV2 | | S; RBD | B-cells; SARS-CoV2 | | ND | |
| | | | | | | | Human Patient | | | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| Ab_511B4 | ND | IGHV4-59 | IGHJ5 | IGKV1D-12 | IGKJ4 | 2144 | ASTYWDSSGY | 3349 | QQANSFRL | Xiaojian Han et al., 2020 |
| | | (Human) | (Human) | (Human) | (Human) | | YYGVDY | | T | (https://www.biorxiv.or |
| | | | | | | | | | | g/content/10.1101/202 |
| | | | | | | | | | | 0.08.19.253369v2.full.p |
| | | | | | | | | | | df+html) |
| Ab_511D11 | ND | IGHV1-18 | IGHJ6 | IGKV3-20 | IGKJ2 | 2145 | AVLDYCSGGS | 3350 | QQYGRSPY | Xiaojian Han et al., 2020 |
| | | (Human) | (Human) | (Human) | (Human) | | SSSGYYNYGM | | T | (https://www.biorxiv.or |
| | | | | | | | DV | | | g/content/10.1101/202 |
| | | | | | | | | | | 0.08.19.253369v2.full.p |
| | | | | | | | | | | df+html) |
| Ab_511E5 | ND | IGHV1-2 | IGHJ2 | IGLV1-40 | IGLJ2 | 2146 | ARDSLFSRVD | 3351 | NSRDSSGN | Xiaojian Han et al., 2020 |
| | | (Human) | (Human) | (Human) | (Human) | | WYFDL | | TVV | (https://www.biorxiv.or |
| | | | | | | | | | | g/content/10.1101/202 |
| | | | | | | | | | | 0.08.19.253369v2.full.p |
| | | | | | | | | | | df+html) |
| Ab_511E7 | ND | IGHV5-51 | IGHJ4 | IGKV1-33 | IGKJ3 | 2147 | ALAVGRGIPT | 3352 | QQYHNLPI | Xiaojian Han et al., 2020 |
| | | (Human) | (Human) | (Human) | (Human) | | SYFDY | | T | (https://www.biorxiv.or |
| | | | | | | | | | | g/content/10.1101/202 |
| | | | | | | | | | | 0.08.19.253369v2.full.p |
| | | | | | | | | | | df+html) |
| Ab_511E9 | ND | IGHV1-18 | IGHJ4 | IGKV6-21 | IGKJ2 | 2148 | AREGAGLIIAY | 3353 | HQSSSLPY | Xiaojian Han et al., 2020 |
| | | (Human) | (Human) | (Human) | (Human) | | DY | | T | (https://www.biorxiv.or |
| | | | | | | | | | | g/content/10.1101/202 |
| | | | | | | | | | | 0.08.19.253369v2.full.p |
| | | | | | | | | | | df+html) |
| Ab_511G5 | ND | IGHV1-46 | IGHJ4 | IGLV1-47 | IGLJ3 | 2149 | ARDGALYSNS | 3354 | TTWDASR | Xiaojian Han et al., 2020 |
| | | (Human) | (Human) | (Human) | (Human) | | PTEFDY | | GGWV | (https://www.biorxiv.or |
| | | | | | | | | | | g/content/10.1101/202 |
| | | | | | | | | | | 0.08.19.253369v2.full.p |
| | | | | | | | | | | df+html) |
| Ab_511G7 | ND | IGHV3-33 | IGHJ4 | IGKV1-33 | IGKJ4 | 2150 | AKGGNYGDY | 3355 | QQYHNVP | Xiaojian Han et al., 2020 |
| | | (Human) | (Human) | (Human) | (Human) | | LRGFDY | | PA | (https://www.biorxiv.or |
| | | | | | | | | | | g/content/10.1101/202 |
| | | | | | | | | | | 0.08.19.253369v2.full.p |
| | | | | | | | | | | df+html) |
| Ab_511H11 | ND | IGHV3-33 | IGHJ6 | IGKV1-39 | IGKJ1 | 2151 | VRGDHSSGW | 3356 | QQSYSSPP | Xiaojian Han et al., 2020 |
| | | (Human) | (Human) | (Human) | (Human) | | YGTYYYYMDV | | WT | (https://www.biorxiv.or |
| | | | | | | | | | | g/content/10.1101/202 |
| | | | | | | | | | | 0.08.19.253369v2.full.p |
| | | | | | | | | | | df+html) |
| Ab_511H7 | ND | IGHV3-23 | IGHJ6 | IGLV1-47 | IGLJ3 | 2152 | ARGLQYYYDT | 3357 | AAWDDSL | Xiaojian Han et al., 2020 |
| | | (Human) | (Human) | (Human) | (Human) | | SGYYKDSYYY | | SGPV | (https://www.biorxiv.or |
| | | | | | | | GVDV | | | g/content/10.1101/202 |
| | | | | | | | | | | 0.08.19.253369v2.full.p |
| | | | | | | | | | | df+html) |
| Ab_51A1 | ND | IGHV3-66 | IGHJ3 | IGKV1-9 | IGKJ5 | 2153 | ARDLNIAGGF | 3358 | QHLNIDPIT | Xiaojian Han et al., 2020 |
| | | (Human) | (Human) | (Human) | (Human) | | DI | | | (https://www.biorxiv.or |
| | | | | | | | | | | g/content/10.1101/202 |
| | | | | | | | | | | 0.08.19.253369v2.full.p |
| | | | | | | | | | | df+html) |
| Ab_51A3 | ND | IGHV1-18 | IGHJ4 | IGKV3-11 | IGKJ5 | 2154 | ARDLAWFGE | 3359 | QQRGNSIT | Xiaojian Han et al., 2020 |
| | | (Human) | (Human) | (Human) | (Human) | | LSESPIEY | | | (https://www.biorxiv.or |
| | | | | | | | | | | g/content/10.1101/202 |
| | | | | | | | | | | 0.08.19.253369v2.full.p |
| | | | | | | | | | | df+html) |
| Ab_51D2 | ND | IGHV2-5 | IGHJ3 | IGKV1-39 | IGKJ4 | 2155 | AHRLAPDYDF | 3360 | QQSYNTLA | 0.08.19.253369v2.full.p |
| | | (Human) | (Human) | (Human) | (Human) | | LTGYYNGDD | | LS | Xiaojian Han et al., 2020 |
| | | | | | | | AFDV | | | (https://www.biorxiv.or |
| | | | | | | | | | | g/content/10.1101/202 |
| | | | | | | | | | | df+html) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| Ab_51D3 | Ab | SARS-CoV2 | | SARS-CoV2 | | S; RBD | B-cells; SARS-CoV2 | | ND | |
| | | | | (weak) | | | Human Patient | | | |
| Ab_51D4 | Ab | SARS-CoV2 | | SARS-CoV2 | | S; RBD | B-cells; SARS-CoV2 | | ND | |
| | | | | (weak) | | | Human Patient | | | |
| Ab_51D7 | Ab | SARS-CoV2 | | SARS-CoV2 | | S; RBD | B-cells; SARS-CoV2 | | ND | |
| | | | | (weak) | | | Human Patient | | | |
| Ab_51E10 | Ab | SARS-CoV2 | | SARS-CoV2 | | S; RBD | B-cells; SARS-CoV2 | | ND | |
| | | | | (weak) | | | Human Patient | | | |
| Ab_51E12 | Ab | SARS-CoV2 | | SARS-CoV2 | | S; RBD | B-cells; SARS-CoV2 | | ND | |
| | | | | | | | Human Patient | | | |
| Ab_51E7 | Ab | SARS-CoV2 | | SARS-CoV2 | | S; RBD | B-cells; SARS-CoV2 | | ND | |
| | | | | | | | Human Patient | | | |
| Ab_51F11 | Ab | SARS-CoV2 | | SARS-CoV2 | | S; RBD | B-cells; SARS-CoV2 | | ND | |
| | | | | | | | Human Patient | | | |
| Ab_52C1 | Ab | SARS-CoV2 | | | | S; RBD | B-cells; SARS-CoV2 | | ND | |
| | | | | | | | Human Patient | | | |
| Ab_52C6 | Ab | SARS-CoV2 | | SARS-CoV2 | | S; RBD | B-cells; SARS-CoV2 | | ND | |
| | | | | | | | Human Patient | | | |
| Ab_52F7 | Ab | SARS-CoV2 | | SARS-CoV2 | | S; RBD | B-cells; SARS-CoV2 | | ND | |
| | | | | (weak) | | | Human Patient | | | |
| Ab_52G9 | Ab | SARS-CoV2 | | SARS-CoV2 | | S; RBD | B-cells; SARS-CoV2 | | ND | |
| | | | | | | | Human Patient | | | |
| Ab_53C10 | Ab | SARS-CoV2 | | | | S; RBD | B-cells; SARS-CoV2 | | ND | |
| | | | | | | | Human Patient | | | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| Ab_51D3 | ND | IGHV3-66 | (Human) | IGKV1-33 | IGKJ2 | 2156 | AREGLLVGPT | 3361 | QQYADLPY | Xiaojian Han et al., 2020 |
| | | (Human) | IGHJ6 | (Human) | (Human) | | GRGLGMDV | | T | (https://www.biorxiv.or |
| | | | | | | | | | | g/content/10.1101/202 |
| | | | | | | | | | | 0.08.19.253369v2.full.p |
| | | | | | | | | | | df+html) |
| Ab_51D4 | ND | IGHV2-70 | IGHJ4 | IGKV1-39 | IGKJ2 | 2157 | ARMVVRGV | 3362 | QQSYSPPH | Xiaojian Han et al., 2020 |
| | | (Human) | (Human) | (Human) | (Human) | | MLDY | | S | (https://www.biorxiv.or |
| | | | | | | | | | | g/content/10.1101/202 |
| | | | | | | | | | | 0.08.19.253369v2.full.p |
| | | | | | | | | | | df+html) |
| Ab_51D7 | ND | IGHV5-51 | IGHJ3 | IGKV1-39 | IGKJ2 | 2158 | ATRTGWTND | 3363 | QQSYSTPC | Xiaojian Han et al., 2020 |
| | | (Human) | (Human) | (Human) | (Human) | | AFDI | | T | (https://www.biorxiv.or |
| | | | | | | | | | | g/content/10.1101/202 |
| | | | | | | | | | | 0.08.19.253369v2.full.p |
| | | | | | | | | | | df+html) |
| Ab_51E10 | ND | IGHV1-18 | IGHJ2 | IGKV4-1 | IGKJ4 | 2159 | ARARQLVLN | 3364 | QQYYITPQ | Xiaojian Han et al., 2020 |
| | | (Human) | (Human) | (Human) | (Human) | | WYFDL | | LT | (https://www.biorxiv.or |
| | | | | | | | | | | g/content/10.1101/202 |
| | | | | | | | | | | 0.08.19.253369v2.full.p |
| | | | | | | | | | | df+html) |
| Ab_51E12 | ND | IGHV3-7 | IGHJ4 | IGKV1-8 | IGKJ2 | 2160 | ARLMYYYGNF | 3365 | QQYYGYPT | Xiaojian Han et al., 2020 |
| | | (Human) | (Human) | (Human) | (Human) | | DY | | | (https://www.biorxiv.or |
| | | | | | | | | | | g/content/10.1101/202 |
| | | | | | | | | | | 0.08.19.253369v2.full.p |
| | | | | | | | | | | df+html) |
| Ab_51E7 | ND | IGHV3-13 | IGHJ2 | IGKV1-39 | IGKJ1 | 2161 | ARVGYYGSGS | 3366 | QQSYSAPP | Xiaojian Han et al., 2020 |
| | | (Human) | (Human) | (Human) | (Human) | | YPLYWYFDL | | WT | (https://www.biorxiv.or |
| | | | | | | | | | | g/content/10.1101/202 |
| | | | | | | | | | | 0.08.19.253369v2.full.p |
| | | | | | | | | | | df+html) |
| Ab_51F11 | ND | IGHV1-69 | IGHJ4 | IGKV3-20 | IGKJ2 | 2162 | ATGRYTYGYG | 3367 | QQYGSSRT | Xiaojian Han et al., 2020 |
| | | (Human) | (Human) | (Human) | (Human) | | YYFDY | | | (https://www.biorxiv.or |
| | | | | | | | | | | g/content/10.1101/202 |
| | | | | | | | | | | 0.08.19.253369v2.full.p |
| | | | | | | | | | | df+html) |
| Ab_52C1 | ND | IGHV3-66 | IGHJ4 | IGLV1-40 | IGLJ3 | 2163 | ARLASDGSGS | 3368 | QSYDSSLS | Xiaojian Han et al., 2020 |
| | | (Human) | (Human) | (Human) | (Human) | | YLDYFDY | | GSWV | (https://www.biorxiv.or |
| | | | | | | | | | | g/content/10.1101/202 |
| | | | | | | | | | | 0.08.19.253369v2.full.p |
| | | | | | | | | | | df+html) |
| Ab_52C6 | ND | IGHV1-69 | IGHJ6 | IGLV8-61 | IGLJ2 | 2164 | ATDGGGGSY | 3369 | VLYMGSGI | Xiaojian Han et al., 2020 |
| | | (Human) | (Human) | (Human) | (Human) | | YYAHYYYGM | | VV | (https://www.biorxiv.or |
| | | | | | | | DV | | | g/content/10.1101/202 |
| | | | | | | | | | | 0.08.19.253369v2.full.p |
| | | | | | | | | | | df+html) |
| Ab_52F7 | ND | IGHV3-9 | IGHJ6 | IGKV2D-30 | IGKJ2 | 2165 | AKDIGVMVP | 3370 | MQGTHW | Xiaojian Han et al., 2020 |
| | | (Human) | (Human) | (Human) | (Human) | | GVTPYGMDV | | PPGT | (https://www.biorxiv.or |
| | | | | | | | | | | g/content/10.1101/202 |
| | | | | | | | | | | 0.08.19.253369v2.full.p |
| | | | | | | | | | | df+html) |
| Ab_52G9 | ND | IGHV3-66 | IGHJ6 | IGKV1-33 | IGKJ1 | 2166 | ARDPMRPG | 3371 | QQYDNLP | Xiaojian Han et al., 2020 |
| | | (Human) | (Human) | (Human) | (Human) | | MDV | | RT | (https://www.biorxiv.or |
| | | | | | | | | | | g/content/10.1101/202 |
| | | | | | | | | | | 0.08.19.253369v2.full.p |
| | | | | | | | | | | df+html) |
| Ab_53C10 | ND | IGHV3-43 | IGHJ4 | IGLV3-21 | IGLJ1 | 2167 | ARESPKLTGY | 3372 | QVWDSSS | Xiaojian Han et al., 2020 |
| | | (Human) | (Human) | (Human) | (Human) | | FDY | | DPYV | (https://www.biorxiv.or |
| | | | | | | | | | | g/content/10.1101/202 |
| | | | | | | | | | | 0.08.19.253369v2.full.p |
| | | | | | | | | | | df+html) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| Ab_53C5 | Ab | SARS-CoV2 | | | | S; RBD | B-cells; SARS-CoV2 | | ND | |
| | | | | | | | Human Patient | | | |
| Ab_53F12 | Ab | SARS-CoV2 | | SARS-CoV2 | | S; RBD | B-cells; SARS-CoV2 | | ND | |
| | | | | (weak) | | | Human Patient | | | |
| Ab_53F9 | Ab | SARS-CoV2 | | | | S; RBD | B-cells; SARS-CoV2 | | ND | |
| | | | | | | | Human Patient | | | |
| Ab_53H3 | Ab | SARS-CoV2 | | SARS-CoV2 | | S; RBD | B-cells; SARS-CoV2 | | ND | |
| | | | | (weak) | | | Human Patient | | | |
| Ab_55A8 | Ab | SARS-CoV2 | | SARS-CoV2 | | S; RBD | B-cells; SARS-CoV2 | | ND | |
| | | | | (weak) | | | Human Patient | | | |
| Ab_55C9 | Ab | SARS-CoV2 | | SARS-CoV2 | | S; RBD | B-cells; SARS-CoV2 | | ND | |
| | | | | (weak) | | | Human Patient | | | |
| Ab_56C12 | Ab | SARS-CoV2 | | SARS-CoV2 | | S; RBD | B-cells; SARS-CoV2 | | ND | |
| | | | | | | | Human Patient | | | |
| Ab_56D7 | Ab | SARS-CoV2 | | SARS-CoV2 | | S; RBD | B-cells; SARS-CoV2 | | ND | |
| | | | | (weak) | | | Human Patient | | | |
| Ab_56E1 | Ab | SARS-CoV2 | | SARS-CoV2 | | S; RBD | B-cells; SARS-CoV2 | | ND | |
| | | | | | | | Human Patient | | | |
| Ab_56H11 | Ab | SARS-CoV2 | | | | S; RBD | B-cells; SARS-CoV2 | | ND | |
| | | | | | | | Human Patient | | | |
| Ab_56H3 | Ab | SARS-CoV2 | | SARS-CoV2 | | S; RBD | B-cells; SARS-CoV2 | | ND | |
| | | | | | | | Human Patient | | | |
| Ab_57A6 | Ab | SARS-CoV2 | | SARS-CoV2 | | S; RBD | B-cells; SARS-CoV2 | | ND | |
| | | | | | | | Human Patient | | | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| Ab_53C5 | ND | IGHV1-69 | IGHJ4 | IGKV1-5 | IGKJ2 | 2168 | ARGRYTYGTE | 3373 | QQYNNLYT | Xiaojian Han et al., 2020 |
| | | (Human) | (Human) | (Human) | (Human) | | GYFDN | | | (https://www.biorxiv.or |
| | | | | | | | | | | g/content/10.1101/202 |
| | | | | | | | | | | 0.08.19.253369v2.full.p |
| | | | | | | | | | | df+html) |
| Ab_53F12 | ND | IGHV3-66 | IGHJ6 | IGKV3-15 | IGKJ2 | 2169 | ARDAVGSYYY | 3374 | QHYNNWP | Xiaojian Han et al., 2020 |
| | | (Human) | (Human) | (Human) | (Human) | | GMEV | | LYT | (https://www.biorxiv.or |
| | | | | | | | | | | g/content/10.1101/202 |
| | | | | | | | | | | 0.08.19.253369v2.full.p |
| | | | | | | | | | | df+html) |
| Ab_53F9 | ND | IGHV3-53 | IGHJ4 | IGKV2D-30 | IGKJ2 | 2170 | AREGLVGTTL | 3375 | MQGTHW | Xiaojian Han et al., 2020 |
| | | (Human) | (Human) | (Human) | (Human) | | TEDY | | PPGT | (https://www.biorxiv.or |
| | | | | | | | | | | g/content/10.1101/202 |
| | | | | | | | | | | 0.08.19.253369v2.full.p |
| | | | | | | | | | | df+html) |
| Ab_53H3 | ND | IGHV3-66 | IGHJ3 | IGKV1-33 | IGKJ3 | 2171 | ARYYGPQGR | 3376 | QQYDNLPL | Xiaojian Han et al., 2020 |
| | | (Human) | (Human) | (Human) | (Human) | | AFDI | | T | (https://www.biorxiv.or |
| | | | | | | | | | | g/content/10.1101/202 |
| | | | | | | | | | | 0.08.19.253369v2.full.p |
| | | | | | | | | | | df+html) |
| Ab_55A8 | ND | IGHV1-69 | IGHJ4 | IGKV1-5 | IGKJ2 | 2172 | ARGTEYGDYD | 3377 | QQYNSYSH | Xiaojian Han et al., 2020 |
| | | (Human) | (Human) | (Human) | (Human) | | VSHD | | T | (https://www.biorxiv.or |
| | | | | | | | | | | g/content/10.1101/202 |
| | | | | | | | | | | 0.08.19.253369v2.full.p |
| | | | | | | | | | | df+html) |
| Ab_55C9 | ND | IGHV3-53 | IGHJ4 | IGKV1-39 | IGKJ2 | 2173 | AREGLVGTAL | 3378 | QQSYSTPP | Xiaojian Han et al., 2020 |
| | | (Human) | (Human) | (Human) | (Human) | | AFDY | | YT | (https://www.biorxiv.or |
| | | | | | | | | | | g/content/10.1101/202 |
| | | | | | | | | | | 0.08.19.253369v2.full.p |
| | | | | | | | | | | df+html) |
| Ab_56C12 | ND | IGHV3-30 | IGHJ5 | IGKV1-39 | IGKJ1 | 2174 | AKDPTSLYCS | 3379 | QQTYSTPR | Xiaojian Han et al., 2020 |
| | | (Human) | (Human) | (Human) | (Human) | | GGSCYNNWF | | T | (https://www.biorxiv.or |
| | | | | | | | DP | | | g/content/10.1101/202 |
| | | | | | | | | | | 0.08.19.253369v2.full.p |
| | | | | | | | | | | df+html) |
| Ab_56D7 | ND | IGHV3-66 | IGHJ6 | IGKV1-9 | IGKJ5 | 2175 | ARDLDYYGM | 3380 | QQLNSYPP | Xiaojian Han et al., 2020 |
| | | (Human) | (Human) | (Human) | (Human) | | DV | | IT | (https://www.biorxiv.or |
| | | | | | | | | | | g/content/10.1101/202 |
| | | | | | | | | | | 0.08.19.253369v2.full.p |
| | | | | | | | | | | df+html) |
| Ab_56E1 | ND | IGHV3-30 | IGHJ3 | IGLV1-44 | IGLJ3 | 2176 | AGGGVLVTS | 3381 | AAWDDSL | Xiaojian Han et al., 2020 |
| | | (Human) | (Human) | (Human) | (Human) | | DPDAFDI | | NGWV | (https://www.biorxiv.or |
| | | | | | | | | | | g/content/10.1101/202 |
| | | | | | | | | | | 0.08.19.253369v2.full.p |
| | | | | | | | | | | df+html) |
| Ab_56H11 | ND | IGHV4-4 | IGHJ4 | IGLV1-44 | IGLJ3 | 2177 | AGEQHIVTTII | 3382 | ATWDDSL | Xiaojian Han et al., 2020 |
| | | (Human) | (Human) | (Human) | (Human) | | DY | | NGRV | (https://www.biorxiv.or |
| | | | | | | | | | | g/content/10.1101/202 |
| | | | | | | | | | | 0.08.19.253369v2.full.p |
| | | | | | | | | | | df+html) |
| Ab_56H3 | ND | IGHV3-66 | IGHJ4 | IGKV3-20 | IGKJ1 | 2178 | ARDYGDYYFD | 3383 | QQYGSSPR | Xiaojian Han et al., 2020 |
| | | (Human) | (Human) | (Human) | (Human) | | Y | | T | (https://www.biorxiv.or |
| | | | | | | | | | | g/content/10.1101/202 |
| | | | | | | | | | | 0.08.19.253369v2.full.p |
| | | | | | | | | | | df+html) |
| Ab_57A6 | ND | IGHV5-51 | IGHJ4 | IGLV3-19 | IGLJ3 | 2179 | ARQESGWSF | 3384 | NSRDSSGN | Xiaojian Han et al., 2020 |
| | | (Human) | (Human) | (Human) | (Human) | | DY | | HWV | (https://www.biorxiv.or |
| | | | | | | | | | | g/content/10.1101/202 |
| | | | | | | | | | | 0.08.19.253369v2.full.p |
| | | | | | | | | | | df+html) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| Ab_57A8 | Ab | SARS-CoV2 | | SARS-CoV2 | | S; RBD | B-cells; SARS-CoV2 | | ND | |
| | | | | | | | Human Patient | | | |
| Ab_57A9 | Ab | SARS-CoV2 | | SARS-CoV2 | | S; RBD | B-cells; SARS-CoV2 | | ND | |
| | | | | | | | Human Patient | | | |
| Ab_57B8 | Ab | SARS-CoV2 | | SARS-CoV2 | | S; RBD | B-cells; SARS-CoV2 | | ND | |
| | | | | (weak) | | | Human Patient | | | |
| Ab_57C4 | Ab | SARS-CoV2 | | SARS-CoV2 | | S; RBD | B-cells; SARS-CoV2 | | ND | |
| | | | | | | | Human Patient | | | |
| Ab_57E11 | Ab | SARS-CoV2 | | SARS-CoV2 | | S; RBD | B-cells; SARS-CoV2 | | ND | |
| | | | | | | | Human Patient | | | |
| Ab_57F7 | Ab | SARS-CoV2 | | SARS-CoV2 | | S; RBD | B-cells; SARS-CoV2 | | ND | |
| | | | | (weak) | | | Human Patient | | | |
| Ab_57G9 | Ab | SARS-CoV2 | | SARS-CoV2 | | S; RBD | B-cells; SARS-CoV2 | | ND | |
| | | | | | | | Human Patient | | | |
| Ab_58A4 | Ab | SARS-CoV2 | | | | S; RBD | B-cells; SARS-CoV2 | | ND | |
| | | | | | | | Human Patient | | | |
| Ab_58D2 | Ab | SARS-CoV2 | | | | S; RBD | B-cells; SARS-CoV2 | | ND | |
| | | | | | | | Human Patient | | | |
| Ab_58G1 | Ab | SARS-CoV2 | | SARS-CoV2 | | S; RBD | B-cells; SARS-CoV2 | | ND | |
| | | | | | | | Human Patient | | | |
| Ab_58G6 | Ab | SARS-CoV2 | | | SARS-CoV2 | S; RBD | B-cells; SARS-CoV2 | | ND | |
| | | | | | | | Human Patient | | | |
| Ab_59A2 | Ab | SARS-CoV2 | | | | S; RBD | B-cells; SARS-CoV2 | | ND | |
| | | | | | | | Human Patient | | | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| Ab_57A8 | ND | IGHV3-23 | IGHJ4 | IGKV1-5 | IGKJ4 | 2180 | AKGQRGSPD | 3385 | QQYNSYSP | Xiaojian Han et al., 2020 |
| | | (Human) | (Human) | (Human) | (Human) | | FFDY | | LT | (https://www.biorxiv.or |
| | | | | | | | | | | g/content/10.1101/202 |
| | | | | | | | | | | 0.08.19.253369v2.full.p |
| | | | | | | | | | | df+html) |
| Ab_57A9 | ND | IGHV1-3 | IGHJ4 | IGKV4-1 | IGKJ1 | 2181 | ARAGWELNY | 3386 | QQYYSFW | Xiaojian Han et al., 2020 |
| | | (Human) | (Human) | (Human) | (Human) | | | | A | (https://www.biorxiv.or |
| | | | | | | | | | | g/content/10.1101/202 |
| | | | | | | | | | | 0.08.19.253369v2.full.p |
| | | | | | | | | | | df+html) |
| Ab_57B8 | ND | IGHV3-53 | IGHJ4 | IGKV1-9 | IGKJ5 | 2182 | ARDLVTWGL | 3387 | QLLNTDPI | Xiaojian Han et al., 2020 |
| | | (Human) | (Human) | (Human) | (Human) | | DY | | T | (https://www.biorxiv.or |
| | | | | | | | | | | g/content/10.1101/202 |
| | | | | | | | | | | 0.08.19.253369v2.full.p |
| | | | | | | | | | | df+html) |
| Ab_57C4 | ND | IGHV3-15 | IGHJ5 | IGKV1-39 | IGKJ4 | 2183 | STTNDYGDYS | 3388 | QQSYSTPL | Xiaojian Han et al., 2020 |
| | | (Human) | (Human) | (Human) | (Human) | | ANY | | T | (https://www.biorxiv.or |
| | | | | | | | | | | g/content/10.1101/202 |
| | | | | | | | | | | 0.08.19.253369v2.full.p |
| | | | | | | | | | | df+html) |
| Ab_57E11 | ND | IGHV1-8 | IGHJ5 | IGLV1-44 | IGLJ3 | 2184 | ARGLWFGDL | 3389 | AAWDDSL | Xiaojian Han et al., 2020 |
| | | (Human) | (Human) | (Human) | (Human) | | TRTKYNWFD | | NGWV | (https://www.biorxiv.or |
| | | | | | | | P | | | g/content/10.1101/202 |
| | | | | | | | | | | 0.08.19.253369v2.full.p |
| | | | | | | | | | | df+html) |
| Ab_57F7 | ND | IGHV4-34 | IGHJ6 | IGLV1-44 | IGLJ3 | 2185 | ARDDSSSSGV | 3390 | AVWDDSL | Xiaojian Han et al., 2020 |
| | | (Human) | (Human) | (Human) | (Human) | | GTGMDV | | NGWV | (https://www.biorxiv.or |
| | | | | | | | | | | g/content/10.1101/202 |
| | | | | | | | | | | 0.08.19.253369v2.full.p |
| | | | | | | | | | | df+html) |
| Ab_57G9 | ND | IGHV2-70 | IGHJ4 | IGKV1-39 | IGKJ2 | 2186 | ARITPHLVYDY | 3391 | QQSYSIPR | Xiaojian Han et al., 2020 |
| | | (Human) | (Human) | (Human) | (Human) | | | | T | (https://www.biorxiv.or |
| | | | | | | | | | | g/content/10.1101/202 |
| | | | | | | | | | | 0.08.19.253369v2.full.p |
| | | | | | | | | | | df+html) |
| Ab_58A4 | ND | IGHV4-59 | IGHJ5 | IGKV3-20 | IGKJ1 | 2187 | ARTLGAYYDIL | 3392 | QQYGSSP | Xiaojian Han et al., 2020 |
| | | (Human) | (Human) | (Human) | (Human) | | TGFRTPGGW | | WT | (https://www.biorxiv.or |
| | | | | | | | FAP | | | g/content/10.1101/202 |
| | | | | | | | | | | 0.08.19.253369v2.full.p |
| | | | | | | | | | | df+html) |
| Ab_58D2 | ND | IGHV3-11 | IGHJ2 | IGKV3-11 | IGKJ2 | 2188 | ASPLLSHNYG | 3393 | QQLGT | Xiaojian Han et al., 2020 |
| | | (Human) | (Human) | (Human) | (Human) | | SGSYYNVYWY | | | (https://www.biorxiv.or |
| | | | | | | | FEL | | | g/content/10.1101/202 |
| | | | | | | | | | | 0.08.19.253369v2.full.p |
| | | | | | | | | | | df+html) |
| Ab_58G1 | ND | IGHV3-53 | IGHJ6 | IGKV1D-12 | IGKJ4 | 2189 | ARDLENGGL | 3394 | QQTNSFPT | Xiaojian Han et al., 2020 |
| | | (Human) | (Human) | (Human) | (Human) | | DV | | | (https://www.biorxiv.or |
| | | | | | | | | | | g/content/10.1101/202 |
| | | | | | | | | | | 0.08.19.253369v2.full.p |
| | | | | | | | | | | df+html) |
| Ab_58G6 | ND | IGHV1-58 | IGHJ3 | IGKV3-20 | IGKJ1 | 2190 | AAPNCNSTTC | 3395 | QQYDNSP | Xiaojian Han et al., 2020 |
| | | (Human) | (Human) | (Human) | (Human) | | HDGFDI | | WT | (https://www.biorxiv.or |
| | | | | | | | | | | g/content/10.1101/202 |
| | | | | | | | | | | 0.08.19.253369v2.full.p |
| | | | | | | | | | | df+html) |
| Ab_59A2 | ND | IGHV3-66 | IGHJ4 | IGKV1-33 | IGKJ3 | 2191 | ARDLPLHGDY | 3396 | QQSDNVP | Xiaojian Han et al., 2020 |
| | | (Human) | (Human) | (Human) | (Human) | | FDY | | VT | (https://www.biorxiv.or |
| | | | | | | | | | | g/content/10.1101/202 |
| | | | | | | | | | | 0.08.19.253369v2.full.p |
| | | | | | | | | | | df+html) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| Ab_59D6 | Ab | SARS-CoV2 | | | | S; RBD | B-cells; SARS-CoV2 | | ND | |
| | | | | | | | Human Patient | | | |
| Ab_81A11 | Ab | SARS-CoV2 | | | | S; RBD | B-cells; SARS-CoV2 | | ND | |
| | | | | | | | Human Patient | | | |
| Ab_81C3 | Ab | SARS-CoV2 | | SARS-CoV2 | | S; RBD | B-cells; SARS-CoV2 | | ND | |
| | | | | (weak) | | | Human Patient | | | |
| Ab_81C7 | Ab | SARS-CoV2 | | | | S; RBD | B-cells; SARS-CoV2 | | ND | |
| | | | | | | | Human Patient | | | |
| Ab_81C8 | Ab | SARS-CoV2 | | SARS-CoV2 | | S; RBD | B-cells; SARS-CoV2 | | ND | |
| | | | | | | | Human Patient | | | |
| Ab_81E1 | Ab | SARS-CoV2 | | SARS-CoV2 | | S; RBD | B-cells; SARS-CoV2 | | ND | |
| | | | | (weak) | | | Human Patient | | | |
| Ab_81E10 | Ab | SARS-CoV2 | | | | S; RBD | B-cells; SARS-CoV2 | | ND | |
| | | | | | | | Human Patient | | | |
| Ab_81F2 | Ab | SARS-CoV2 | | | | S; RBD | B-cells; SARS-CoV2 | | ND | |
| | | | | | | | Human Patient | | | |
| Ab_82B6 | Ab | SARS-CoV2 | | | | S; RBD | B-cells; SARS-CoV2 | | ND | |
| | | | | | | | Human Patient | | | |
| Ab_82C6 | Ab | SARS-CoV2 | | | | S; RBD | B-cells; SARS-CoV2 | | ND | |
| | | | | | | | Human Patient | | | |
| Ab_82F6 | Ab | SARS-CoV2 | | SARS-CoV2 | | S; RBD | B-cells; SARS-CoV2 | | ND | |
| | | | | | | | Human Patient | | | |
| Acharya | Ab | HIV | | | | S Glycans | B-cells; HIV Human | | Various | |
| et al., | | Glycans, | | | | | Patient | | | |
| 2020 | | SARS-CoV2 | | | | | | | | |
| | | Glycans | | | | | | | | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| Ab_59D6 | ND | IGHV4-34 | IGHJ4 | IGLV1-40 | IGLJ2 | 2192 | ARHRRDYITM | 3397 | QSYDSALV | Xiaojian Han et al., 2020 |
| | | (Human) | (Human) | (Human) | (Human) | | IVRPTRLWAF | | V | (https://www.biorxiv.or |
| | | | | | | | DY | | | g/content/10.1101/202 |
| | | | | | | | | | | 0.08.19.253369v2.full.p |
| | | | | | | | | | | df+html) |
| Ab_81A11 | ND | IGHV1-69 | IGHJ2 | IGLV1-40 | IGLJ2 | 2193 | AREAGTTDW | 3398 | QSYDSSLS | Xiaojian Han et al., 2020 |
| | | (Human) | (Human) | (Human) | (Human) | | YFDL | | VVV | (https://www.biorxiv.or |
| | | | | | | | | | | g/content/10.1101/202 |
| | | | | | | | | | | 0.08.19.253369v2.full.p |
| | | | | | | | | | | df+html) |
| Ab_81C3 | ND | IGHV4-39 | IGHJ4 | IGLV2-14 | IGLJ1 | 2194 | ARHPRFSWR | 3399 | SSFTSSSTP | Xiaojian Han et al., 2020 |
| | | (Human) | (Human) | (Human) | (Human) | | GNDSGYFDY | | YV | (https://www.biorxiv.or |
| | | | | | | | | | | g/content/10.1101/202 |
| | | | | | | | | | | 0.08.19.253369v2.full.p |
| | | | | | | | | | | df+html) |
| Ab_81C7 | ND | IGHV2-5 | IGHJ4 | IGLV1-36 | IGLJ3 | 2195 | AHSMVRGVL | 3400 | AAWDDSL | Xiaojian Han et al., 2020 |
| | | (Human) | (Human) | (Human) | (Human) | | FGADFDY | | NGPV | (https://www.biorxiv.or |
| | | | | | | | | | | g/content/10.1101/202 |
| | | | | | | | | | | 0.08.19.253369v2.full.p |
| | | | | | | | | | | df+html) |
| Ab_81C8 | ND | IGHV3-33 | IGHJ4 | IGKV1-39 | IGKJ1 | 2196 | ARDGVDFGM | 3401 | QQSYNTPP | Xiaojian Han et al., 2020 |
| | | (Human) | (Human) | (Human) | (Human) | | VTLFDY | | WT | (https://www.biorxiv.or |
| | | | | | | | | | | g/content/10.1101/202 |
| | | | | | | | | | | 0.08.19.253369v2.full.p |
| | | | | | | | | | | df+html) |
| Ab_81E1 | ND | IGHV1-24 | IGHJ4 | IGLV1-47 | IGLJ2 | 2197 | AITSVARGLR | 3402 | AAWDDSL | Xiaojian Han et al., 2020 |
| | | (Human) | (Human) | (Human) | (Human) | | GYFDT | | SRVV | (https://www.biorxiv.or |
| | | | | | | | | | | g/content/10.1101/202 |
| | | | | | | | | | | 0.08.19.253369v2.full.p |
| | | | | | | | | | | df+html) |
| Ab_81E10 | ND | IGHV1-24 | IGHJ4 | IGLV1-47 | IGLJ2 | 2198 | AITSLARGLKG | 3403 | AAWDDSL | Xiaojian Han et al., 2020 |
| | | (Human) | (Human) | (Human) | (Human) | | YFDS | | SGVI | (https://www.biorxiv.or |
| | | | | | | | | | | g/content/10.1101/202 |
| | | | | | | | | | | 0.08.19.253369v2.full.p |
| | | | | | | | | | | df+html) |
| Ab_81F2 | ND | IGHV4-34 | IGHJ5 | IGKV2-28 | IGKJ4 | 2199 | ARGWTVPPL | 3404 | MQALQTP | Xiaojian Han et al., 2020 |
| | | (Human) | (Human) | (Human) | (Human) | | WVLNWFDP | | RT | (https://www.biorxiv.or |
| | | | | | | | | | | g/content/10.1101/202 |
| | | | | | | | | | | 0.08.19.253369v2.full.p |
| | | | | | | | | | | df+html) |
| Ab_82B6 | ND | IGHV4-61 | IGHJ5 | IGKV2-30 | IGKJ1 | 2200 | AMTYYDYIW | 3405 | CMQGTH | Xiaojian Han et al., 2020 |
| | | (Human) | (Human) | (Human) | (Human) | | GRVDPQFDP | | WPPT | (https://www.biorxiv.or |
| | | | | | | | | | | g/content/10.1101/202 |
| | | | | | | | | | | 0.08.19.253369v2.full.p |
| | | | | | | | | | | df+html) |
| Ab_82C6 | ND | IGHV4-39 | IGHJ4 | IGKV2-30 | IGKJ2 | 2201 | ARFITDGYSS | 3406 | MQGTHW | Xiaojian Han et al., 2020 |
| | | (Human) | (Human) | (Human) | (Human) | | GSDS | | PMYT | (https://www.biorxiv.or |
| | | | | | | | | | | g/content/10.1101/202 |
| | | | | | | | | | | 0.08.19.253369v2.full.p |
| | | | | | | | | | | df+html) |
| Ab_82F6 | ND | IGHV3-30 | IGHJ4 | IGKV1-33 | IGKJ4 | 2202 | AKQASPYCSG | 3407 | QQHDNVP | Xiaojian Han et al., 2020 |
| | | (Human) | (Human) | (Human) | (Human) | | GSCYSGNFDY | | LT | (https://www.biorxiv.or |
| | | | | | | | | | | g/content/10.1101/202 |
| | | | | | | | | | | 0.08.19.253369v2.full.p |
| | | | | | | | | | | df+html) |
| Acharya | Various | Various | Various | Various | Various | | Various | | Various | Priyamvada Acharya et |
| et al., | | | | | | | | | | al., 2020 |
| 2020 | | | | | | | | | | (https://www.biorxiv.or |
| | | | | | | | | | | g/content/10.1101/202 |
| | | | | | | | | | | 0.06.30.178897v1); Wilt |
| | | | | | | | | | | on Williams et al., 2020 |
| | | | | | | | | | | (https://www.biorxiv.or |
| | | | | | | | | | | g/content/10.1101/202 |
| | | | | | | | | | | 0.06.30.178921v1) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| B38 | Ab | SARS-CoV2 | | SARS-CoV2 | | S; RBD | B-cells; SARS-CoV2 | 124 | EVQLVESGGGLVQPGGSLRLSCAASGFI | 1206 |
| | | | | | | | Human Patient | | VSSNYMSWVRQAPGKGLEWVSVIYSG | |
| | | | | | | | | | GSTYYADSVKGRFTISRHNSKNTLYLQM | |
| | | | | | | | | | NSLRAEDTAVYYCAREAYGMDVWGQG | |
| | | | | | | | | | TTVTVSS | |
| BD-236 | Ab | SARS-CoV2 | | SARS-CoV2 | | S; RBD | B-cells; SARS-CoV2 | 125 | EVQLVESGGGLIQPGGSLRLSCAASGITV | |
| | | | | | | | Human Patient | | SSNYMSWVRQAPGKGLEWVSVIYSGG | |
| | | | | | | | | | STDYADSVKGRFTISRDKSKNTLYLQMN | |
| | | | | | | | | | SLRAEDTAVYYCARDLGEAGGMDVWG | |
| | | | | | | | | | QGTTVTVSS | |
| BD-368-2 | Ab | SARS-CoV2 | | SARS-CoV2 | | S; RBD | B-cells; SARS-CoV2 | | ND | |
| | | | | | | | Human Patient | | | |
| BD-494 | Ab | SARS-CoV2 | | SARS-CoV2 | | S; | Phage Display | | ND | |
| | | | | | | probably | Library (Antibody, | | | |
| | | | | | | RBD | human, immune- | | | |
| | | | | | | (implied | CoV2) | | | |
| | | | | | | by | | | | |
| | | | | | | clustering) | | | | |
| BD-498 | Ab | SARS-CoV2 | | SARS-CoV2 | | S; | Phage Display | | ND | |
| | | | | | | probably | Library (Antibody, | | | |
| | | | | | | RBD | human, immune- | | | |
| | | | | | | (implied | CoV2) | | | |
| | | | | | | by | | | | |
| | | | | | | clustering) | | | | |
| BD-500 | Ab | SARS-CoV2 | | SARS-CoV2 | | S; | B-cells; SARS-CoV2 | | ND | |
| | | | | | | probably | Human Patient | | | |
| | | | | | | RBD | | | | |
| | | | | | | (implied | | | | |
| | | | | | | by | | | | |
| | | | | | | custering) | | | | |
| BD-503 | Ab | SARS-CoV2 | | SARS-CoV2 | | S; RBD | B-cells; SARS-CoV2 | | ND | |
| | | | | | | | Human Patient | | | |
| BD-504 | Ab | SARS-CoV2 | | SARS-CoV2 | | S; RBD | B-cells; SARS-CoV2 | | ND | |
| | | | | | | | Human Patient | | | |
| BD-505 | Ab | SARS-CoV2 | | SARS-CoV2 | | S; RBD | B-cells; SARS-CoV2 | | ND | |
| | | | | | | | Human Patient | | | |
| BD-506 | Ab | SARS-CoV2 | | SARS-CoV2 | | S; RBD | B-cells; SARS-CoV2 | | ND | |
| | | | | | | | Human Patient | | | |
| BD-507 | Ab | SARS-CoV2 | | SARS-CoV2 | | S; RBD | B-cells; SARS-CoV2 | | ND | |
| | | | | | | | Human Patient | | | |
| BD-508 | Ab | SARS-CoV2 | | SARS-CoV2 | | S; RBD | B-cells; SARS-CoV2 | | ND | |
| | | | | | | | Human Patient | | | |
| BD-515 | Ab | SARS-CoV2 | | SARS-CoV2 | | S; RBD | B-cells; SARS-CoV2 | | ND | |
| | | | | | | | Human Patient | | | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| B38 | DIVMTQSPSFLSASVGDRVTITCRASQ | IGHV3-53 | IGHJ6 | IGKV1-9 | IGKJ2 | 2203 | AREAYGMDV | 3408 | QQLNSYPP | Yan Wu et al., 2020 |
| | GISSYLAWYQQKPGKAPKLLIYAASTL | (Human) | (Human) | (Human) | (Human) | | | | YT | (https://science.science |
| | QSGVPSRFSGSGSGTEFTLTISSLQPED | | | | | | | | | mag.org/content/early/ |
| | FATYYCQQLNSYPPYTFGQGTKLEIK | | | | | | | | | 2020/05/12/science.abc |
| | | | | | | | | | | 2241) |
| BD-236 | ND | IGHV3-53 | IGHJ6 | ND | ND | 2204 | ARDLGEAGG | | ND | Shuo Du et al., 2020 |
| | | (Human) | (Human) | | | | MDV | | | (https://www.biorxiv.or |
| | | | | | | | | | | g/content/10.1101/202 |
| | | | | | | | | | | 0.07.09.195263v1) |
| BD-368-2 | ND | ND | ND | ND | ND | | ND | | ND | Shuo Du et al., 2020 |
| | | | | | | | | | | (https://www.biorxiv.or |
| | | | | | | | | | | g/content/10.1101/202 |
| | | | | | | | | | | 0.07.09.195263v1) |
| BD-494 | ND | IGHV3-53 | IGHJ6 | IGKV1-9 | IGKJ3 | 2205 | ARDLVVYGM | 3409 | QQLNSYPF | Yunlong Cao et al., 2020 |
| | | (Human) | (Human) | (Human) | (Human) | | DV | | T | (https://www.sciencedi |
| | | | | | | | | | | rect.com/science/articl |
| | | | | | | | | | | e/X/S00928674203062 |
| | | | | | | | | | | 06) |
| BD-498 | ND | IGHV3-66 | IGHJ6 | IGKV1-9 | IGKJ5 | 2206 | ARDLVVYGM | 3410 | QQLNSYPL | Yunlong Cao et al., 2020 |
| | | (Human) | (Human) | (Human) | (Human) | | DV | | T | (https://www.sciencedi |
| | | | | | | | | | | rect.com/science/articl |
| | | | | | | | | | | e/pii/S00928674203062 |
| | | | | | | | | | | 06) |
| BD-500 | ND | IGHV3-53 | IGHJ6 | IGKV1-39 | IGKJ5 | 2207 | ARDAMSYG | 3411 | QQSYSTPP | Yunlong Cao et al., 2020 |
| | | (Human) | (Human) | (Human) | (Human) | | MDV | | DT | (https://www.sciencedi |
| | | | | | | | | | | rect.com/science/articl |
| | | | | | | | | | | e/pii/S00928674203062 |
| | | | | | | | | | | 06) |
| BD-503 | ND | IGHV3-53 | IGHJ6 | IGKV1-39 | IGKJ3 | 2208 | ARDAAVYGID | 3412 | QQSYTTPL | Yunlong Cao et al., 2020 |
| | | (Human) | (Human) | (Human) | (Human) | | V | | FT | (https://www.sciencedi |
| | | | | | | | | | | rect.com/science/articl |
| | | | | | | | | | | e/pii/S00928674203062 |
| | | | | | | | | | | 06) |
| BD-504 | ND | IGHV3-66 | IGHJ6 | IGKV1-9 | IGKJ3 | 2209 | ARDLISRGMD | 3413 | QQSYTTPL | Yunlong Cao et al., 2020 |
| | | (Human) | (Human) | (Human) | (Human) | | V | | FT | (https://www.sciencedi |
| | | | | | | | | | | rect.com/science/articl |
| | | | | | | | | | | e/pii/S00928674203062 |
| | | | | | | | | | | 06) |
| BD-505 | ND | IGHV3-53 | IGHJ6 | IGKV1-33 | IGKJ5 | 2210 | ARDRVVYGM | 3414 | HQYDNLPP | Yunlong Cao et al., 2020 |
| | | (Human) | (Human) | (Human) | (Human) | | DV | | T | (https://www.sciencedi |
| | | | | | | | | | | rect.com/science/articl |
| | | | | | | | | | | e/pii/S00928674203062 |
| | | | | | | | | | | 06) |
| BD-506 | ND | IGHV3-53 | IGHJ6 | IGKV1-9 | IGKJ4 | 2211 | ARDLVSYGM | 3415 | QQLNSYPL | Yunlong Cao et al., 2020 |
| | | (Human) | (Human) | (Human) | (Human) | | DV | | T | (https://www.sciencedi |
| | | | | | | | | | | rect.com/science/articl |
| | | | | | | | | | | e/pii/S00928674203062 |
| | | | | | | | | | | 06) |
| BD-507 | ND | IGHV3-53 | IGHJ6 | IGKV1-9 | IGKJ3 | 2212 | ARDLVVYGM | 3416 | QQLNSNP | Yunlong Cao et al., 2020 |
| | | (Human) | (Human) | (Human) | (Human) | | DV | | PIT | (https://www.sciencedi |
| | | | | | | | | | | rect.com/science/articl |
| | | | | | | | | | | e/pii/S00928674203062 |
| | | | | | | | | | | 06) |
| BD-508 | ND | IGHV3-53 | IGHJ6 | IGKV1-39 | IGKJ2 | 2213 | ARDAQNYG | 3417 | QQSYSTPP | Yunlong Cao et al., 2020 |
| | | (Human) | (Human) | (Human) | (Human) | | MDV | | YT | (https://www.sciencedi |
| | | | | | | | | | | rect.com/science/articl |
| | | | | | | | | | | e/pii/S00928674203062 |
| | | | | | | | | | | 06) |
| BD-515 | ND | IGHV3-66 | IGHJ4 | IGKV1-39 | IGKJ5 | | ND | | ND | Yunlong Cao et al., 2020 |
| | | (Human) | (Human) | (Human) | (Human) | | | | | (https://www.sciencedi |
| | | | | | | | | | | rect.com/science/articl |
| | | | | | | | | | | e/pii/S00928674203062 |
| | | | | | | | | | | 06) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| BD-604 | Ab | SARS-CoV2 | | SARS-CoV2 | | S; RBD | B-cells; SARS-CoV2 | 126 | EVQLVESGGGLIQPGGSLRLSCAASGIIV | |
| | | | | | | | Human Patient | | SSNYMTWVRQAPGKGLEWVSVIYSGG | |
| | | | | | | | | | STFYADSVKGRFTISRDNSKNTLYLQMSS | |
| | | | | | | | | | LRAEDTAVYYCARDLGPYGMDVWGQG | |
| | | | | | | | | | TTVTVSS | |
| BD-629 | Ab | SARS-CoV2 | | SARS-CoV2 | | S; RBD | B-cells; SARS-CoV2 | 127 | EVQLVESGGGLIQPGGSLRLSCAASEFIV | |
| | | | | | | | Human Patient | | SRNYMSWVRQAPGKGLEWVSVIYSGG | |
| | | | | | | | | | STYYADSVKGRFTISRDNSKNTLNLQMN | |
| | | | | | | | | | SLRAEDTAVYYCARDYGDYYFDYWGQG | |
| | | | | | | | | | TLVTVSS | |
| BD23 | Ab | SARS-CoV2 | | SARS-CoV2 | | S; RBD | B-cells; SARS-CoV2 | 128 | QVQLVQSGSELKKPGASVKVSCKASGYT | 1207 |
| | | | | | | | Human Patient | | FTSYAMNWVRQAPGQGLEWMGWINT | |
| | | | | | | | | | NTGNPTYAQGFTGRFVFSLDTSVSTAYL | |
| | | | | | | | | | QISSLKAEDTAVYYCARPQGGSSWYRDY | |
| | | | | | | | | | YYGMDVWGQGTTVTVSS | |
| Bertoglio | Ab | SARS-CoV2 | | SARS-CoV2 | | S; RBD | Phage Display | | 17 | |
| et al., | | | | | | | Library (Antibody, | | | |
| 2020 | | | | | | | human, non- | | | |
| | | | | | | | immune) | | | |
| C002 | Ab | SARS-CoV2 | SARS- | SARS-CoV2 | SARS-CoV1 | S; RBD | B-cells; SARS-CoV2 | 129 | EVQLVESGGGVVQPGRSLRLSCAASGFT | 1208 |
| | | | CoV1 | | | | Human Patient | | FSIYGMHWVRQAPGKGLEWVAVISYD | |
| | | | | | | | | | GSNKYYADSVKGRFTISRDNSKNTLYLQ | |
| | | | | | | | | | MNSLRAEDTAVYYCAKEGRPSDIVVVVA | |
| | | | | | | | | | FDYWGQGTLVTVSS | |
| C003 | Ab | SARS-CoV2 | SARS- | SARS-CoV2 | SARS-CoV1 | S; RBD | B-cells; SARS-CoV2 | 130 | EVQLVESGGGLIQPGGSLRLSCAASGFT | 1209 |
| | | | CoV1 | (weak) | | | Human Patient | | VSSNYMSWVRQAPGKGLEWVSVIYSG | |
| | | | | | | | | | GSTYYADSVKGRFTISRDNSKNTLYLQM | |
| | | | | | | | | | NSLRAGDTAVYYCARDYGDFYFDYWGQ | |
| | | | | | | | | | GTLVTVSS | |
| C004 | Ab | SARS-CoV2 | SARS- | SARS-CoV2 | SARS-CoV1 | S; RBD | B-cells; SARS-CoV2 | 131 | QVQLVQSGAEVKKPGASVKVSCKASGY | 1210 |
| | | | CoV1 | | | | Human Patient | | TFTGYYMHWVRQAPGQGLEWMGWI | |
| | | | | | | | | | NPISGGTNYAQKFQGRVTMTRDTSISTA | |
| | | | | | | | | | YMELSRLRSDDTAVYYCASPASRGYSGY | |
| | | | | | | | | | DHGYYYYMDVWGKGTTVTVSS | |
| C005 | Ab | SARS-CoV2 | SARS- | SARS-CoV2 | SARS-CoV1 | S; RBD | B-cells; SARS-CoV2 | 132 | QVQLVQSGPEVKKPGTSVKVSCKASGFT | 1211 |
| | | | CoV1 | | | | Human Patient | | FTSSAVQWVRQARGQRLEWIGWIVVG | |
| | | | | | | | | | SGNTNYAQKFQERVTITRDMSTSTAYM | |
| | | | | | | | | | ELSSLRSEDTAVYYCAAPHCSGGSCLDAF | |
| | | | | | | | | | DIWGQGTMVTVSS | |
| C006 | Ab | SARS-CoV2 | SARS- | SARS-CoV2 | SARS-CoV1 | S; RBD | B-cells; SARS-CoV2 | 133 | QVQLVESGGGLVKPGGSLRLSCAASGFI | 1212 |
| | | | CoV1 | (weak) | | | Human Patient | | FSDYCMSWIRRAPGKGLEWLSYISNSGT | |
| | | | | | | | | | TRYYADSVKGRFTISRDNGRNSLYLQMD | |
| | | | | | | | | | SLSAEDTAVYYCARRGDGSSSIYYYNYM | |
| | | | | | | | | | DVWGKGTTVTVSS | |
| C008 | Ab | SARS-CoV2 | SARS- | SARS-CoV2 | SARS-CoV1 | S; RBD | B-cells; SARS-CoV2 | 134 | EVQLVESGGGVVQPGRSLRLSCAASGFT | 1213 |
| | | | CoV1 | (weak) | | | Human Patient | | FSSYGMHWVRQAPGKGLEWVTVISYD | |
| | | | | | | | | | GRNKYYADSVKGRFTISRDNSKNTLYLQ | |
| | | | | | | | | | MNSLRAEDTAVYYCAREFGDPEWYFDY | |
| | | | | | | | | | WGQGTLVTVSS | |
| C009 | Ab | SARS-CoV2 | SARS- | SARS-CoV2 | SARS-CoV1 | S; RBD | B-cells; SARS-CoV2 | 135 | QVQLVQSGAEVKKPGASVKVSCMASGY | 1214 |
| | | | CoV1 | | | | Human Patient | | TFTGYYMHWVRQAPGQGLEWMGWI | |
| | | | | | | | | | NPNSGGTNYAQKFQGRVTMTRDTSIST | |
| | | | | | | | | | AYMELSRLRSDDTAVYYCARDSPFSALG | |
| | | | | | | | | | ASNDYWGQGTLVTVSS | |
| C010 | Ab | SARS-CoV2 | SARS- | | SARS-CoV1, | S; RBD | B-cells; SARS-CoV2 | 136 | EVQLVESGGGVVQPGRSLRLSCAASGFT | 1215 |
| | | | CoV1 | | SARS-CoV2 | | Human Patient | | FSSYAMHWVRQAPAKGLEWVAVILYD | |
| | | | | | | | | | GSGKYYADSVKGRFTISRDNSKNTLYLQ | |
| | | | | | | | | | MNSLRAEDTAVYYCARDGIVDTALVTW | |
| | | | | | | | | | FDYWGQGTLVTVSS | |
| C013 | Ab | SARS-CoV2 | SARS- | SARS-CoV2 | SARS-CoV1 | S; RBD | B-cells; SARS-CoV2 | 137 | QVQLVQSGAEVKKPGSSVKVSCKASGG | 1216 |
| | | | CoV1 | (weak) | | | Human Patient | | TFSSYAISWVRQAPGQGLEWMGGIIPIF | |
| | | | | | | | | | GTANYAQKFQGRVTITADESTSTAYMEL | |
| | | | | | | | | | SSLRSEDTAVYYCARGNRLLYCSSTSCYL | |
| | | | | | | | | | DAVRQGYYYYYYMDVWGKGTTVTVSS | |
| C016 | Ab | SARS-CoV2 | SARS- | | SARS-CoV1, | S; RBD | B-cells; SARS-CoV2 | 138 | EVQLVESGGGVVQPGRSLRLSCAASGFT | 1217 |
| | | | CoV1 | | SARS-CoV2 | | Human Patient | | FSRYGMHWVRQAPGKGLEWVAVISYD | |
| | | | | | | | | | GSNKYYADSVKGRFTISRDNSKNTLYLQ | |
| | | | | | | | | | MNSLRAEDTAVYYCAKVTAPYCSGGSCY | |
| | | | | | | | | | GGNFDYWGQGTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| BD-604 | ND | IGHV3-53 | IGHJ6 | IGKV1-9 | IGKJ2 | 2214 | ARDLGPYGM | | ND | Shuo Du et al., 2020 |
| | | (Human) | (Human) | (Human) | (Human) | | DV | | | (https://www.biorxiv.or |
| | | | | | | | | | | g/content/10.1101/202 |
| | | | | | | | | | | 0.07.09.195263v1) |
| BD-629 | ND | IGHV3-53 | IGHJ4 | IGKV3-20 | IGKJ1 | 2215 | ARDYGDYYFD | | ND | Shuo Du et al., 2020 |
| | | (Human) | (Human) | (Human) | (Human) | | Y | | | (https://www.biorxiv.or |
| | | | | | | | | | | g/content/10.1101/202 |
| | | | | | | | | | | 0.07.09.195263v1) |
| BD23 | DIQMTQSPSTLSASVGDRVTITCRASQ | IGHV7-4- | IGHJ6 | IGKV1-5 | IGKJ2 | 2216 | ARPQGGSSW | 3418 | QQYNSYPY | Yunlong Cao et al., 2020 |
| | SISSWLAWYQQKPGKAPKLLIYKASSL | 1 | (Human) | (Human) | (Human) | | YRDYYYGMD | | T | (https://www.sciencedi |
| | ESGVPSRFSGSGSGTEFTLTISSLQPDD | (Human) | | | | | V | | | rect.com/science/articl |
| | FATYYCQQYNSYPYTFGQGTKLEIK | | | | | | | | | e/pii/S00928674203062 |
| | | | | | | | | | | 06) |
| Bertoglio | 17 | Various | Various | Various | Various | | Various | | Various | Federico Bertoglio et |
| et al., | | | | | | | | | | al., 2020 |
| 2020 | | | | | | | | | | (https://www.biorxiv.or |
| | | | | | | | | | | g/content/10.1101/202 |
| | | | | | | | | | | 0.06.05.135921v1) |
| C002 | DIQLTQSPSSLSASVGDRVTITCRASQS | IGHV3-30 | IGHJ4 | IGKV1-39 | IGKJ1 | 2217 | AKEGRPSDIV | 3419 | QQSYSTPR | Davide Robbiani et al., |
| | ISSYLNWYQQKPGKAPKLLIYAASSLQS | (Human) | (Human) | (Human) | (Human) | | VVVAFDY | | T | 2020 |
| | GVPSRFSGSGSGTDFTLTISSLQPEDFA | | | | | | | | | (https://www.nature.co |
| | TYYCQQSYSTPRTFGQGTKVEIK | | | | | | | | | m/articles/s41586-020- |
| | | | | | | | | | | 2456-9) |
| C003 | EIVLTQSPGTLSLSPGERATLSCRASQS | IGHV3-53 | IGHJ4 | IGKV3-20 | IGKJ2 | 2218 | ARDYGDFYFD | 3420 | QQYGSSPR | Davide Robbiani et al., |
| | VSSTYLAWYQQKPGQAPRLLIYGASSR | (Human) | (Human) | (Human) | (Human) | | Y | | T | 2020 |
| | ATGIPDRFSGSGSGTDFTLTISRLEPED | | | | | | | | | (https://www.nature.co |
| | FAVYYCQQYGSSPRTFGQGTKLEIK | | | | | | | | | m/articles/s41586-020- |
| | | | | | | | | | | 2456-9) |
| C004 | AIRMITQSPSSLSASVGDRVTITCQASQ | IGHV1-2 | IGHJ6 | IGKV1-33 | IGKJ5 | 2219 | ASPASRGYSG | 3421 | QQYDNLPI | Davide Robbiani et al. |
| | DISNYLNWYQQKPGKAPKLLIYDASNL | (Human) | (Human) | (Human) | (Human) | | YDHGYYYYM | | T | 2020 |
| | ETGVPSRFSGSGSGTDFTFTISSLQPED | | | | | | DV | | | (https://www.nature.co |
| | IATYYCQQYDNLPITFGQGTRLEIK | | | | | | | | | m/articles/s41586-020- |
| | | | | | | | | | | 2456-9) |
| C005 | EIVLTQSPGTLSLSPGERATLSCRASQS | IGHV1-58 | IGHJ3 | IGKV3-20 | IGKJ1 | 2220 | AAPHCSGGSC | 3422 | QQYGSSP | Davide Robbiani et al., |
| | VRSSYLAWYQQKPGQAPRLLIYGASSR | (Human) | (Human) | (Human) | (Human) | | LDAFDI | | WT | 2020 |
| | ATGIPDRFSGSGSGTDFTLTISRLEPED | | | | | | | | | (https://www.nature.co |
| | FAVYYCQQYGSSPWTFGQGTKVEIK | | | | | | | | | m/articles/s41586-020- |
| | | | | | | | | | | 2456-9) |
| C006 | QSVLTQPPSASGTPGQRVTVSCSGSSS | IGHV3-11 | IGHJ6 | IGLV1-44 | IGLJ3 | 2221 | ARRGDGSSSI | 3423 | AAWDDSL | Davide Robbiani et al., |
| | NIGSNTVNWYQQLPGTAPKLLIYSNN | (Human) | (Human) | (Human) | (Human) | | YYYNYMDV | | NGPV | 2020 |
| | QRPSGVPDRFSGSKSGTSASLAISGLQ | | | | | | | | | (https://www.nature.co |
| | SEDEADYFCAAWDDSLNGPVFGGGT | | | | | | | | | m/articles/s41586-020- |
| | KLTVL | | | | | | | | | 2456-9) |
| C008 | DIQMTQSPSTLSASVGDRVTITCRANQ | IGHV3-30 | IGHJ4 | IGKV1-5 | IGKJ1 | 2222 | AREFGDPEW | 3424 | QQYNSYW | Davide Robbiani et al., |
| | SISSWLAWYQQKPGKAPKLLIYKASSL | (Human) | (Human) | (Human) | (Human) | | YFDY | | T | 2020 |
| | ESGVPSRFSGSGSGTEFTLTISSLQPDD | | | | | | | | | (https://www.nature.co |
| | FATYYCQQYNSYWTFGQGTKVEIK | | | | | | | | | m/articles/s41586-020- |
| | | | | | | | | | | 2456-9) |
| C009 | QSALTQPPSASGSPGQSVTISCTGTSS | IGHV1-2 | IGHJ4 | IGLV2-8 | IGLJ3 | 2223 | ARDSPFSALG | 3425 | SSDAGSNN | Davide Robbiani et al., |
| | DVGGYNYVSWYQQHPGKAPKLMIYE | (Human) | (Human) | (Human) | (Human) | | ASNDY | | VV | 2020 |
| | VSKRPSGVPDRFSGSKSGNTASLTVSG | | | | | | | | | (https://www.nature.co |
| | LQAEDEAEYYCSSDAGSNNVVFGGGT | | | | | | | | | m/articles/s41586-020- |
| | KLTVL | | | | | | | | | 2456-9) |
| C010 | DIQLTQSPSSLSASVGDRVTITCRASQS | IGHV3-30 | IGHJ4 | IGKV1-39 | IGKJ1 | 2224 | ARDGIVDTAL | 3426 | QQSYSTPP | Davide Robbiani et al., |
| | ISTYLNWYQQKPGKAPKLLIYAASSLQS | (Human) | (Human) | (Human) | (Human) | | VTWFDY | | WT | 2020 |
| | GVPSRFSGSGSGTDFTLTISSLQPEDFA | | | | | | | | | (https://www.nature.co |
| | TYYCQQSYSTPPWTFGQGTKVEIK | | | | | | | | | m/articles/s41586-020- |
| | | | | | | | | | | 2456-9) |
| C013 | EIVLTQSPATLSLSPGERATLSCRASQS | IGHV1-69 | IGHJ6 | IGKV3-11 | IGKJ4 | 2225 | ARGNRLLYCS | 3427 | QQRSNWP | Davide Robbiani et al., |
| | VSSYLAWYQQKPGQAPRLLIYDASNR | (Human) | (Human) | (Human) | (Human) | | STSCYLDAVR | | LT | 2020 |
| | ATGIPARFSGSGSGTDFTLTISSLEPEDF | | | | | | QGYYYYYYM | | | (https://www.nature.co |
| | AVYYCQQRSNWPLTFGGGTKVEIK | | | | | | DV | | | m/articles/s41586-020- |
| | | | | | | | | | | 2456-9) |
| C016 | AIRMTQSPSSLSASVGDRVTITCQASQ | IGHV3-30 | IGHJ4 | IGKV1-33 | IGKJ4 | 2226 | AKVTAPYCSG | 3428 | QQYDNLP | Davide Robbiani et al., |
| | DISNYLNWYQQKPGKAPKLLIYDASNL | (Human) | (Human) | (Human) | (Human) | | GSCYGGNFDY | | PT | 2020 |
| | ETGVPSRFSGSGSGTDFTFTINSLQPED | | | | | | | | | (https://www.nature.co |
| | IATYYCQQYDNLPPTFGGGTKVEIK | | | | | | | | | m/articles/s41586-020- |
| | | | | | | | | | | 2456-9) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| C017 | Ab | SARS-CoV2 | SARS- | SARS-CoV2 | SARS-CoV1 | S; RBD | B-cells; SARS-CoV2 | 139 | EVQLVESGGGLVQPGRSLRLSCAASGFT | 1218 |
| | | | CoV1 | | | | Human Patient | | FDDYAMHWVRQAPGKGLEWVSGISW | |
| | | | | | | | | | NSGTIGYADSVKGRFTISRDNAKNSLYLQ | |
| | | | | | | | | | MNSLRAEDTAFYYCAKAGVRGIAAAGP | |
| | | | | | | | | | DLNFDHWGQGTLVTVSS | |
| C018 | Ab | SARS-CoV2 | SARS- | | SARS-CoV1, | S; RBD | B-cells; SARS-CoV2 | 140 | EVQLVESGGGVVQPGRSLRLSCAASGFT | 1219 |
| | | | CoV1 | | SARS-CoV2 | | Human Patient | | FSNYAIHWVRQAPGKGLEWVAVISYDG | |
| | | | | | | | | | SNKYYADSVKGRFTISRDNSKNTLYLQM | |
| | | | | | | | | | NSLRAEDTAVYYCARDFDDSSFWAFDY | |
| | | | | | | | | | WGQGTLVTVSS | |
| C019 | Ab | SARS-CoV2 | SARS- | | SARS-CoV1, | S; RBD | B-cells; SARS-CoV2 | 141 | QVQLVQSGAEVKKPGASVKVSCKASGY | 1220 |
| | | | CoV1 | | SARS-CoV2 | | Human Patient | | TFTSYYMHWVRQAPGQGLEWMGIINP | |
| | | | | | | | | | SGGSTSYAQKFQGRVTMTRDTSTSTVY | |
| | | | | | | | | | MELSSLRSEDTAVYYCARVPREGTPGFD | |
| | | | | | | | | | PWGQGTLVTVSS | |
| C021 | Ab | SARS-CoV2 | SARS- | | SARS-CoV1, | S; RBD | B-cells; SARS-CoV2 | 142 | QVQLQESGPGLVKPSQTLSLTCTVSGGSI | 1221 |
| | | | CoV1 | | SARS-CoV2 | | Human Patient | | SSGGYYWSWIRQHPGKGLEWIGYIYYS | |
| | | | | | | | | | GSTYYNPSLKSRVTISVDTSKNQFSLKLSS | |
| | | | | | | | | | VTAADTAVYYCARVWQYYDSSGSFDYW | |
| | | | | | | | | | GQGTLVTVSS | |
| C022 | Ab | SARS-CoV1, | | SARS-CoV2 | | S; RBD | B-cells; SARS-CoV2 | 143 | QVQLQESGPGLVKPSETLSVTCTVSGGSI | 1222 |
| | | SARS-CoV2 | | | | | Human Patient | | SSSRYYWGWIRQPPGKGLEWIGSIYYSG | |
| | | | | | | | | | STYYNPSLKSRVTISVDTSKNQFSLKLSSV | |
| | | | | | | | | | TAADTAVYYCARHAAAYYDRSGYYFIEYF | |
| | | | | | | | | | QHWGQGTLVTVSS | |
| C027 | Ab | SARS-CoV1 | | | SARS-CoV2 | S; RBD | B-cells; SARS-CoV2 | 144 | EVQLVESGGGVVQPGRSLRLSCAASGFT | 1223 |
| | | (weak), | | | | | Human Patient | | FSSYGMHWVRQAPGKGLEWVAVISYD | |
| | | SARS-CoV2 | | | | | | | GSNKYYADSVKGRFTISRDNSKNTLYLQ | |
| | | | | | | | | | MNSLRAEDTAVYYCAKASGIYCSGGDCY | |
| | | | | | | | | | SYYFDYWGQGTLVTVSS | |
| C029 | Ab | SARS-CoV2 | SARS- | | SARS-CoV1, | S; RBD | B-cells; SARS-CoV2 | 145 | QVQLQESGPGLVKPSQTLSLTCTVSGGSI | 1224 |
| | | | CoV1 | | SARS-CoV2 | | Human Patient | | SSGGYYWSWIRQHPGKGLEWIGYIYYS | |
| | | | | | | | | | GSTYYNPSLKSRVTISVDTSKNQFSLKLSS | |
| | | | | | | | | | VTAADTAVYYCARTMYYYDSSGSFDYW | |
| | | | | | | | | | GQGTLVTVSS | |
| C030 | Ab | SARS-CoV2 | SARS- | | SARS-CoV1, | S; RBD | B-cells; SARS-CoV2 | 146 | EVQLVESGGGVVQPGRSLRLSCAASGFT | 1225 |
| | | | CoV1 | | SARS-CoV2 | | Human Patient | | FSSYGMHWVRQAPGKGLEWVAVISYD | |
| | | | | | | | | | GSNKYYADSVKGRFTISRDNSKNTLYLQ | |
| | | | | | | | | | MNSLRAEDTAVYYCAKASGIYCSGGNCY | |
| | | | | | | | | | SYYFDYWGQGTLVTVSS | |
| C031 | Ab | SARS-CoV2 | SARS- | | SARS-CoV1, | S; RBD | B-cells; SARS-CoV2 | 147 | EVQLVESGGGLVQPGGSLRLSCAASGFT | 1226 |
| | | | CoV1 | | SARS-CoV2 | | Human Patient | | FSSYDMHWVRQATGKGLEWVSAIGTA | |
| | | | | | | | | | GDTYYPGSVKGRFTISRENAKNSLYLQM | |
| | | | | | | | | | NSLRAGDTAVYYCARVGYDSSGYSGWY | |
| | | | | | | | | | FDLWGRGTLVTVSS | |
| C101 | Ab | SARS-CoV2 | SARS- | SARS-CoV2 | SARS-CoV1 | S; RBD | B-cells; SARS-CoV2 | 148 | QVQLVESGGGLIQPGGSLRLSCAASGFI | 1227 |
| | | | CoV1 | | | | Human Patient | | VSSNYMSWVRQAPGKGLEWVSVIYSG | |
| | | | | | | | | | GSTFYTDSVKGRFTISRDNSKNTLYLQM | |
| | | | | | | | | | NSLRAEDTAVYYCVRDYGDFYFDYWGQ | |
| | | | | | | | | | GTLVTVSS | |
| C102 | Ab | SARS-CoV2 | SARS- | SARS-CoV2 | SARS-CoV1 | S; RBD | B-cells; SARS-CoV2 | 149 | QVQLVESGGGLIQPGGSLRLSCAASGFI | 1228 |
| | | | CoV1 | | | | Human Patient | | VSSNYMSWVRQAPGKGLEWVSVIYSG | |
| | | | | | | | | | GSTFYADSVKGRFTISRDNSKNTLYLQM | |
| | | | | | | | | | NSLRAEDTAVYYCARDYGDYYFDYWGQ | |
| | | | | | | | | | GTLVTVSS | |
| C103 | Ab | SARS-CoV2 | SARS- | SARS-CoV2 | SARS-CoV1 | S; RBD | B-cells; SARS-CoV2 | 150 | QVQLQQWGAGLLKPSETLSLTCAVSGG | 1229 |
| | | | CoV1 | | | | Human Patient | | SLSGFYWTWIRQPPGKGLEWIGETNHF | |
| | | | | | | | | | GSTGYKPSLKSRVTISVDMSRNQFSLKVT | |
| | | | | | | | | | SVTAADTAVYYCARKPLLYSDFSPGAFDI | |
| | | | | | | | | | WGQGTMVTVSS | |
| C104 | Ab | SARS-CoV2 | SARS- | SARS-CoV2 | SARS-CoV1 | S; RBD | B-cells; SARS-CoV2 | 151 | QVQLQQWGAGLLKPSETLSLSCAVYGG | 1230 |
| | | | CoV1 | | | | Human Patient | | SLSGYYWSWIRQPPGKGLEWIGEINHF | |
| | | | | | | | | | GSTGYNPSLKSRVTISVDTSKSQFSVKLSS | |
| | | | | | | | | | VTAADTAVYYCARKPLLYSNLSPGAFDI | |
| | | | | | | | | | WGQGTMVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| C017 | EIVLTQSPATLSLSPGERATLSCRASQS | IGHV3-9 | IGHJ4 | IGKV3-11 | IGKJ5 | 2227 | AKAGVRGIAA | 3429 | QQRIT | Davide Robbiani et al., |
| | VSSYLAWYQQKPGQAPRLLIYDASNR | (Human) | (Human) | (Human) | (Human) | | AGPDLNFDH | | | 2020 |
| | ATGIPARFSGSGSGTDFTLTISSLEPEDF | | | | | | | | | (https://www.nature.co |
| | AVYYCQQRITFGQGTRLEIK | | | | | | | | | m/articles/s41586-020- |
| | | | | | | | | | | 2456-9) |
| C018 | DIQLTQSPSSLSASVGDRVTITCRASQS | IGHV3-30 | IGHJ4 | IGKV1-39 | IGKJ2 | 2228 | ARDFDDSSF | 3430 | QQSYSTPP | Davide Robbiani et al., |
| | IRSYLNWYQQKPGKAPKLLIYAASSLQ | (Human) | (Human) | (Human) | (Human) | | WAFDY | | AT | 2020 |
| | SGVPSRFSGSGSGTDFTLTISSLQPDDF | | | | | | | | | (https://www.nature.co |
| | ATYYCQQSYSTPPATFGQGTKLEIK | | | | | | | | | m/articles/s41586-020- |
| | | | | | | | | | | 2456-9) |
| C019 | SYELTQPPSVSVAPGKTARITCGENNI | IGHV1-46 | IGHJ5 | IGLV3-21 | IGLJ3 | 2229 | ARVPREGTPG | 3431 | QVWDSSS | Davide Robbiani et al., |
| | GSKSVHWYQQKPGQAPVLVIYYDSDR | (Human) | (Human) | (Human) | (Human) | | FDP | | DHVV | 2020 |
| | PSGIPERFSGSNSGNTATLTINRVEAG | | | | | | | | | (https://www.nature.co |
| | DEADYYCQVWDSSSDHVVFGGGTKL | | | | | | | | | m/articles/s41586-020- |
| | TVL | | | | | | | | | 2456-9) |
| C021 | DIVMTQSPLSLPVTPGEPASISCRSSQS | IGHV4-31 | IGHJ4 | IGKV2-28 | IGKJ3 | 2230 | ARVWQYYDS | 3432 | MQALQTP | Davide Robbiani et al., |
| | LLHSNGYNYLDWYLQKPGQSPQLLIYL | (Human) | (Human) | (Human) | (Human) | | SGSFDY | | FT | 2020 |
| | GSNRASGVPDRFSGSGSGTDFTLKISR | | | | | | | | | (https://www.nature.co |
| | VEAEDVGVYYCMQALQTPFTFGPGTK | | | | | | | | | m/articles/s41586-020- |
| | VDIK | | | | | | | | | 2456-9) |
| C022 | DIQMTQSPSTLSASVGDSVTITCRASQ | IGHV4-39 | IGHJ1 | IGKV1-5 | IGKJ2 | 2231 | ARHAAAYYDR | 3433 | QQYNNYR | Davide Robbiani et al., |
| | SISSWLAWYQQKPGKAPKLLIYKASSL | (Human) | (Human) | (Human) | (Human) | | SGYYFIEYFQH | | YT | 2020 |
| | ESGVPSRFSGSGSGTEFTLTISSLQPDD | | | | | | | | | (https://www.nature.co |
| | FATYYCQQYNNYRYTFGQGTKLEIK | | | | | | | | | m/articles/s41586-020- |
| | | | | | | | | | | 2456-9) |
| C027 | DIQMTQSPSTLSASVGDRVTITCRASQ | IGHV3-30 | IGHJ4 | IGKV1-5 | IGKJ1 | 2232 | AKASGIYCSG | 3434 | QQYNSYST | Davide Robbiani et al., |
| | SISSWLAWYQQKPGKAPKLLIYKASSL | (Human) | (Human) | (Human) | (Human) | | GDCYSYYFDY | | | 2020 |
| | ESGVPSRFSGSGSGTEFTLTISSLQPDD | | | | | | | | | (https://www.nature.co |
| | FATYYCQQYNSYSTFGQGTKVEIK | | | | | | | | | m/articles/s41586-020- |
| | | | | | | | | | | 2456-9) |
| C029 | DIVMTQSPLSLPVTPGEPASISCRSSQS | IGHV4-31 | IGHJ4 | IGKV2-28 | IGKJ4 | 2233 | ARTMYYYDSS | 3435 | MQALQTP | Davide Robbiani et al., |
| | LLHSNGYNYLDWYLQKPGQSPQLLIYL | (Human) | (Human) | (Human) | (Human) | | GSFDY | | HT | 2020 |
| | GSNRASGVPDRFSGSGSGTDFTLKISR | | | | | | | | | (https://www.nature.co |
| | VEAEDVGVYYCMQALQTPHTFGGGT | | | | | | | | | m/articles/s41586-020- |
| | KVEIK | | | | | | | | | 2456-9) |
| C030 | DIQMTQSPSTLSASVGDRVTITCRASQ | IGHV3-30 | IGHJ4 | IGKV1-5 | IGKJ1 | 2234 | AKASGIYCSG | 3436 | QQYNSYST | Davide Robbiani et al., |
| | SISSWLAWYQQKPGKAPKLLIYKASSL | (Human) | (Human) | (Human) | (Human) | | GNCYSYYFDY | | | 2020 |
| | ESGVPSRFSGSGSGTEFTLTISSLQPDD | | | | | | | | | (https://www.nature.co |
| | FATYYCQQYNSYSTFGQGTKVEIK | | | | | | | | | m/articles/s41586-020- |
| | | | | | | | | | | 2456-9) |
| C031 | DIQMTQSPSSLSASVGDRVTITCRASQ | IGHV3-13 | IGHJ2 | IGKV1-39 | IGKJ4 | 2235 | ARVGYDSSGY | 3437 | QQSYSTPP | Davide Robbiani et al., |
| | SISSYLNWYQQKPGKAPKVLIYAASSL | (Human) | (Human) | (Human) | (Human) | | SGWYFDL | | LT | 2020 |
| | QSGVPSRFSGSGSGTDFTLTISSLQPED | | | | | | | | | (https://www.nature.co |
| | FATYYCQQSYSTPPLTFGGGTKVEIK | | | | | | | | | m/articles/s41586-020- |
| | | | | | | | | | | 2456-9) |
| C101 | EIVLTQSPGTLSLSPGERATLSCRASQS | IGHV3-53 | IGHJ4 | IGKV3-20 | IGKJ1 | 2236 | VRDYGDFYFD | 3438 | QQYGSSPR | m/articles/s41586-020- |
| | VSSSYLAWYQQKPGQAPRLLIYGASSR | (Human) | (Human) | (Human) | (Human) | | Y | | T | Davide Robbiani et al., |
| | ATGIPDRFSGGGSETDFTLTISRLEPED | | | | | | | | | 2020 |
| | CAVYYCQQYGSSPRTFGQGTKVEIK | | | | | | | | | (https://www.nature.co |
| | | | | | | | | | | 2456-9) |
| C102 | EIVLTQSPGTLSLSPGERATLSCRASQS | IGHV3-53 | IGHJ4 | IGKV3-20 | IGKJ1 | 2237 | ARDYGDYYFD | 3439 | QQYGSSPR | Davide Robbiani et al., |
| | VSSSYLAWYQQKPGQAPRLLIYGASSR | (Human) | (Human) | (Human) | (Human) | | Y | | T | 2020 |
| | ATGIPDRFSGSGSGTDFTLTISRLEPED | | | | | | | | | (https://www.nature.co |
| | FAVYYCQQYGSSPRTFGQGTKVEIK | | | | | | | | | m/articles/s41586-020- |
| | | | | | | | | | | 2456-9) |
| C103 | EIVLTQSPGTLSLSPGERATLSCRASQT | IGHV4-34 | IGHJ3 | IGKV3-20 | IGKJ4 | 2238 | ARKPLLYSDFS | 3440 | QQYTTTPR | Davide Robbiani et al., |
| | VTANYLAWYQQKPGQAPRLLIYGASK | (Human) | (Human) | (Human) | (Human) | | PGAFDI | | T | 2020 |
| | RATGIPDRFSGSGSGTDFTLSISRLEPE | | | | | | | | | (https://www.nature.co |
| | DFAVYYCQQYTTTPRTFGGGTKVEIK | | | | | | | | | m/articles/s41586-020- |
| | | | | | | | | | | 2456-9) |
| C104 | EIVLTQSPGTVSLSPGERATLSCWASQ | IGHV4-34 | IGHJ3 | IGKV3-20 | IGKJ4 | 2239 | ARKPLLYSNLS | 3441 | QQYGTTPR | Davide Robbiani et al., |
| | SVSASYLAWYQQKPGQAPRLLIYGASS | (Human) | (Human) | (Human) | (Human) | | PGAFDI | | T | 2020 |
| | RATGIPDRFSGSGSGTDFTLTISRLEPE | | | | | | | | | (https://www.nature.co |
| | DFAVYYCQQYGTTPRTFGGGTKVEIK | | | | | | | | | m/articles/s41586-020- |
| | | | | | | | | | | 2456-9) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| C105 | Ab | SARS-CoV2 | SARS- | SARS-CoV2 | SARS-CoV1 | S; RBD | B-cells; SARS-CoV2 | 152 | QVQLVESGGGLIQPGGSLRLSCAASGFT | 1231 |
| | | | CoV1 | | | | Human Patient | | VSSNYMSWVRQAPGKGLEWVSVIYSG | |
| | | | | | | | | | GSTYYADSVKGRFTISRDNSKNTLYLQM | |
| | | | | | | | | | NSLRAEDTAVYYCARGEGWELPYDYWG | |
| | | | | | | | | | QGTLVTVSS | |
| C106 | Ab | SARS-CoV1, | | | SARS-CoV2 | S; RBD | B-cells; SARS-CoV2 | 153 | QLQLQESGPGLVKPSETLSLTCTVSGASV | 1232 |
| | | SARS-CoV2 | | | | | Human Patient | | SSGSYYWSWIRQPPGKGLEWIGYIYYSG | |
| | | | | | | | | | STNYNPSLKSRVTISVDTSKNQFSLKLSSV | |
| | | | | | | | | | TAADTAVYYCARERPGGTYSNTWYTPT | |
| | | | | | | | | | DTNWFDTWGQGTLVTVSS | |
| C107 | Ab | SARS-CoV2 | SARS- | | SARS-CoV1, | S; RBD | B-cells; SARS-CoV2 | 154 | QVQLVQSGAEVKKPGASVRVSCKASGY | 1233 |
| | | | CoV1 | | SARS-CoV2 | | Human Patient | | TFTSYGFSWVRQAPGQGLEWMGWISA | |
| | | | | | | | | | YNGNTNFAQKLQGRVTMTTDTSTSTAY | |
| | | | | | | | | | MELRSLRSDDTAVYYCARGEAVAGTTGF | |
| | | | | | | | | | FDYWGQGTLVTVSS | |
| C108 | Ab | SARS-CoV2 | SARS- | SARS-CoV2 | SARS-CoV1 | S; RBD | B-cells; SARS-CoV2 | 155 | QVQLQESGPGLVKPSGTLSLTCAVSGGS | 1234 |
| | | | CoV1 | (weak) | | | Human Patient | | ISSTNWWSWVRQPPGKGLEWIGEIYHT | |
| | | | | | | | | | GSTNYNPSLKSRVTISVDKSKNQFSLKLSS | |
| | | | | | | | | | VTAADTAVYYCVRDGGRPGDAFDIWG | |
| | | | | | | | | | QGTMVTVSS | |
| C109 | Ab | SARS-CoV2 | | | | S; RBD | B-cells; SARS-CoV2 | 156 | EVQLVESGGGLVQPGGSLRLSCAASGFT | 1235 |
| | | | | | | | Human Patient | | FSSYWMSWVRQAPGKGLEWVANIKQ | |
| | | | | | | | | | DGSEKYYVDSVKGRFTISGDNAKNSLYL | |
| | | | | | | | | | HMNSLRAEDTAVYYCAIQLWLRGGYDY | |
| | | | | | | | | | WGQGTLVTVSS | |
| C110 | Ab | SARS-CoV2 | SARS- | SARS-CoV2 | SARS-CoV1 | S; RBD | B-cells; SARS-CoV2 | 157 | QVQLQQSGAEVKKPGESLKISCKGSGYS | 1236 |
| | | | CoV1 | | | | Human Patient | | FTSYWIGWVRQMPGKGLEWMGIIYPG | |
| | | | | | | | | | DSDTRYSPSFQGQVTISADKSISTAYMQ | |
| | | | | | | | | | WSSLKASDTAMYYCARSFRDDPRIAVA | |
| | | | | | | | | | GPADAFDIWGQGTMVTVSS | |
| C111 | Ab | SARS-CoV2 | | | | S; RBD | B-cells; SARS-CoV2 | 158 | QLQLQESGPGLVKPSETLSLTCTVSGGSI | 1237 |
| | | | | | | | Human Patient | | SSYYWSWIRQPPGKGLEWIGYIYYSGST | |
| | | | | | | | | | NYNPSLKSRVTISVDTSKNQFSLKLSSVTA | |
| | | | | | | | | | ADTAVYYCARVEDWGYCSSTNCYSGAF | |
| | | | | | | | | | DIWGQGTMVTVSS | |
| C112 | Ab | SARS-CoV2 | SARS- | SARS-CoV2 | SARS-CoV1 | S; RBD | B-cells; SARS-CoV2 | 159 | QVQLVESGGGVVQPGRSLRLSCAASGF | 1238 |
| | | | CoV1 | (weak) | | | Human Patient | | TFSSHAMHWVRQAPGKGLEWVAVISY | |
| | | | | | | | | | DGSNKYYADSVKGRFTISRDNSKNTLYL | |
| | | | | | | | | | QMNSLRAEDTAVYYCAREDYYDSSGSF | |
| | | | | | | | | | DYWGQGTLVTVSS | |
| C113 | Ab | SARS-CoV2 | SARS- | | SARS-CoV1, | S; RBD | B-cells; SARS-CoV2 | 160 | QVQLVESGGGVVQPGRSLRLSCAASGF | 1239 |
| | | | CoV1 | | SARS-CoV2 | | Human Patient | | TFSNFGMHWVRQAPGKGLEWVAVIW | |
| | | | | | | | | | YDGSNKYYADSVKGRFTISRDNSKNTLYL | |
| | | | | | | | | | QMNSLRAEDTAVYYCARGVNPDDILTG | |
| | | | | | | | | | VDAFDIWGQGTMVTVSS | |
| C114 | Ab | SARS-CoV2 | SARS- | | SARS-CoV1, | S; RBD | B-cells; SARS-CoV2 | 161 | QVQLVESGGGLIQPGGSLKLSCVVSGFT | 1240 |
| | | | CoV1 | | SARS-CoV2 | | Human Patient | | VSKNYISWVRQAPGKGLEWVSVIFAGG | |
| | | | | | | | | | STFYADSVKGRFAISRDNSNNTLFLQMN | |
| | | | | | | | | | SLRVEDTAIYYCARGDGELFFDQWGQG | |
| | | | | | | | | | TLVTVSS | |
| C115 | Ab | SARS-CoV2 | SARS- | SARS-CoV2 | SARS-CoV1 | S; RBD | B-cells; SARS-CoV2 | 162 | QVQLVESGGGLIKPGRSLRLSCTASGFTF | 1241 |
| | | | CoV1 | (weak) | | | Human Patient | | GDYAMTWFRQAPGKGLEWVGFIRSKA | |
| | | | | | | | | | YGGTTGYAASVKYRFTISRDDSKSIAYLQ | |
| | | | | | | | | | MDSLKTEDTAVYYCTRWDGWSQHDY | |
| | | | | | | | | | WGQGTLVTVSS | |
| C116 | Ab | SARS-CoV2 | SARS- | | SARS-CoV1, | S; RBD | B-cells; SARS-CoV2 | 163 | QVQLVESGGGVVQPGRSLRLSCAASGF | 1242 |
| | | | CoV1 | | SARS-CoV2 | | Human Patient | | TYSTYAMHWVRQAPGKGLEWVAFISYD | |
| | | | | | | | | | GSNKYYADSVKGRFTISRDNSKNTLYLQ | |
| | | | | | | | | | MNSLRAEDTAVYYCARDFYHNWFDPW | |
| | | | | | | | | | GQGTLVTVSS | |
| C117 | Ab | SARS-CoV2 | SARS- | SARS-CoV2 | SARS-CoV1 | S; RBD | B-cells; SARS-CoV2 | 164 | QVQLVESGGGVVQPGRSLRLSCAASGF | 1243 |
| | | | CoV1 | (weak) | | | Human Patient | | TFSTYAMHWVRQAPGEGLEWVAVISY | |
| | | | | | | | | | DGSNTYYADSVKGRFTISRDNSKNTLYL | |
| | | | | | | | | | QMNSLRAEDTAVYYCARDPIWFGELLSP | |
| | | | | | | | | | PFVHFDYWGQGTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| C105 | QSALTQPPSASGSPGQSVTISCTGTSS | IGHV3-53 | IGHJ4 | IGLV2-8 | IGLJ3 | 2240 | ARGEGWELP | 3442 | SSYEGSNN | Davide Robbiani et al., |
| | DVGGYKYVSWYQQHPGKAPKLMIYE | (Human) | (Human) | (Human) | (Human) | | YDY | | FVV | 2020 |
| | VSKRPSGVPDRFSGSKSGNTASLTVSG | | | | | | | | | (https://www.nature.co |
| | LQAEDEADYYCSSYEGSNNFVVFGGG | | | | | | | | | m/articles/s41586-020- |
| | TKLTVL | | | | | | | | | 2456-9); Christopher |
| | | | | | | | | | | Barnes et al., 2020 |
| | | | | | | | | | | (https://www.sciencedi |
| | | | | | | | | | | rect.com/science/articl |
| | | | | | | | | | | e/pii/S00928674203075 |
| | | | | | | | | | | 71) |
| C106 | SYELTQPPSVSVAPGKTARITCGGNNI | IGHV4-61 | IGHJ4 | IGLV3-21 | IGLJ3 | 2241 | ARERPGGTYS | 3443 | QVWDSSR | Davide Robbiani et al., |
| | GSKSVHWYQQKPGQAPVLVIYFDSDR | (Human) | (Human) | (Human) | (Human) | | NTWYTPTDT | | DHVV | 2020 |
| | PSGIPERFSGSNSGNTATLTISRVEAGD | | | | | | NWFDT | | | (https://www.nature.co |
| | EADYYCQVWDSSRDHVVFGGGTKLT | | | | | | | | | m/articles/s41586-020- |
| | VL | | | | | | | | | 2456-9) |
| C107 | QSVLTQPPSASGTPGQRVTISCSGSSS | IGHV1-18 | IGHJ4 | IGLV1-47 | IGLJ3 | 2242 | ARGEAVAGTT | 3444 | AAWDDSL | Davide Robbiani et al., |
| | NIGSNYVYWYQQLPGTAPKLLIYRNN | (Human) | (Human) | (Human) | (Human) | | GFFDY | | SGFVV | 2020 |
| | QRPSGVPDRFSGSKSGTSASLAISGLRS | | | | | | | | | (https://www.nature.co |
| | EDEADYYCAAWDDSLSGFVVFGGGTK | | | | | | | | | m/articles/s41586-020- |
| | LTVI | | | | | | | | | 2456-9) |
| C108 | QSALTQPASVSGSPGQSITISCTGTSSD | IGHV4-4 | IGHJ3 | IGLV2-14 | IGLJ1 | 2243 | VRDGGRPGD | 3445 | NSYTSSSTR | Davide Robbiani et al., |
| | VGGYNYVSWYQQHPGKAPKLMIYDV | (Human) | (Human) | (Human) | (Human) | | AFDI | | V | 2020 |
| | SNRPSGVSNRFSGSKSGNTASLTISGL | | | | | | | | | (https://www.nature.co |
| | QAEDEADYYCNSYTSSSTRVFGTGTKV | | | | | | | | | m/articles/s41586-020- |
| | TVL | | | | | | | | | 2456-9) |
| C109 | QSALTQPPSASGSPGQSVTISCTGTSS | IGHV3-7 | IGHJ4 | IGLV2-8 | IGLJ3 | 2244 | AIQLWLRGGY | 3446 | SSYAGSNN | Davide Robbiani et al., |
| | DVGGYNYVSWYQQHPGKAPKLMIYE | (Human) | (Human) | (Human) | (Human) | | DY | | YVV | 2020 |
| | VTKRPSGVPDRFSGSKSGNTASLTVSG | | | | | | | | | (https://www.nature.co |
| | LQAEDEADYYCSSYAGSNNYVVFGGG | | | | | | | | | m/articles/s41586-020- |
| | TKLTVL | | | | | | | | | 2456-9) |
| C110 | DIQMTQSPSTLSASVGDRVTITCRASQ | IGHV5-51 | IGHJ3 | IGKV1-5 | IGKJ2 | 2245 | ARSFRDDPRI | 3447 | QQYNSYPY | Davide Robbiani et al., |
| | SISYWLAWYQQKPGKAPKLLIYQASSL | (Human) | (Human) | (Human) | (Human) | | AVAGPADAF | | T | 2020 |
| | ESGVPSRFSGSESGTEFTLTISSLQPDD | | | | | | DI | | | (https://www.nature.co |
| | FATYYCQQYNSYPYTFGQGTKLEIK | | | | | | | | | m/articles/s41586-020- |
| | | | | | | | | | | 2456-9) |
| C111 | QSVLTQPPSVSEAPRQRVTISCSGSSS | IGHV4-59 | IGHJ3 | IGLV1-36 | IGLJ3 | 2246 | ARVEDWGYC | 3448 | AAWDDSL | Davide Robbiani et al., |
| | NIGNNAVNWYQQVPGKAPKLLIYYDD | (Human) | (Human) | (Human) | (Human) | | SSTNCYSGAF | | NGAWV | 2020 |
| | LLPSGVSDRFSGSKSGTSASLAISGLQS | | | | | | DI | | | (https://www.nature.co |
| | EDEADYYCAAWDDSLNGAWVFGGG | | | | | | | | | m/articles/s41586-020- |
| | TKLTVL | | | | | | | | | 2456-9) |
| C112 | QSALTQPASVSGSPGQSITISCTGTSSD | IGHV3-30 | IGHJ4 | IGLV2-14 | IGLJ3 | 2247 | AREDYYDSSG | 3449 | SSYTSSST | Davide Robbiani et al., |
| | VGGYNYVSWYQQHPGKAPKLMIYDV | (Human) | (Human) | (Human) | (Human) | | SFDY | | WV | 2020 |
| | SNRPSGVSNRFSGSKSGNTASLTISGL | | | | | | | | | (https://www.nature.co |
| | QAEDEADYYCSSYTSSSTWVFGGGTKL | | | | | | | | | m/articles/s41586-020- |
| | TVL | | | | | | | | | 2456-9) |
| C113 | DIQMTQSPSTLSASVGDRVTITCRASQ | IGHV3-33 | IGHJ3 | IGKV1-5 | IGKJ4 | 2248 | ARGVNPDDIL | 3450 | QQHNSSPL | Davide Robbiani et al., |
| | SMSSWLAWYQQKPGNAPKLLIYKASS | (Human) | (Human) | (Human) | (Human) | | TGVDAFDI | | T | 2020 |
| | LESGVPSRFSGSGSGTEFTLTISSLQPD | | | | | | | | | (https://www.nature.co |
| | DFATYYCQQHNSSPLTFGGGTKVEIK | | | | | | | | | m/articles/s41586-020- |
| | | | | | | | | | | 2456-9) |
| C114 | QSVLTQPPSVSGAPGQRVTISCTGTSS | IGHV3-53 | IGHJ4 | IGLV1-40 | IGLJ3 | 2249 | ARGDGELFFD | 3451 | QSYDSSLY | Davide Robbiani et al., |
| | NIGAGYDVHWYQQLPGRAPKVLISGN | (Human) | (Human) | (Human) | (Human) | | Q | | AV | 2020 |
| | NIRPSEVPDRFSGSRSGTSASLAITSLQ | | | | | | | | | (https://www.nature.co |
| | PEDEAQYYCQSYDSSLYAVFGGGTKLT | | | | | | | | | m/articles/s41586-020- |
| | VL | | | | | | | | | 2456-9) |
| C115 | DIVMTQSPLSLSVTPGEPASISCRSSQS | IGHV3-49 | IGHJ4 | IGKV2-28 | IGKJ2 | 2250 | TRWDGWSQ | 3452 | MQVLQIPY | Davide Robbiani et al., |
| | LLHSNGNNYFDWYLQKPGQSPQLLIY | (Human) | (Human) | (Human) | (Human) | | HDY | | T | 2020 |
| | LGSNRASGVPDRFSGSGSGTDFTLKIS | | | | | | | | | (https://www.nature.co |
| | RVEAEDVGVYYCMQVLQIPYTFGQGT | | | | | | | | | m/articles/s41586-020- |
| | KLEIK | | | | | | | | | 2456-9) |
| C116 | NFMLTQPHSVSESPGKTVTISCTGSSG | IGHV3-30 | IGHJ5 | IGLV6-57 | IGLJ3 | 2251 | ARDFYHNWF | 3453 | QSYDSGN | Davide Robbiani et al., |
| | SIASNYVQWYQQRPGSAPTTVIYEDN | (Human) | (Human) | (Human) | (Human) | | DP | | HWVV | 2020 |
| | QRPSGVPDRFSGSIDRSSNSASLTISGL | | | | | | | | | (https://www.nature.co |
| | KTEDEADYYCQSYDSGNHWVVFGGG | | | | | | | | | m/articles/s41586-020- |
| | TRLTVL | | | | | | | | | 2456-9) |
| C117 | QSVLTQPPSVSAAPGQKVTISCSGSSS | IGHV3-30 | IGHJ4 | IGLV1-51 | IGLJ1 | 2252 | ARDPIWFGEL | 3454 | GAWDSSLS | Davide Robbiani et al., |
| | NIGNNLVSWYQQLPGTAPKLLIYENN | (Human) | (Human) | (Human) | (Human) | | LSPPFVHFDY | | AGGVYV | 2020 |
| | KRPSGIPDRFSGSKSGTSATLGITGLQT | | | | | | | | | (https://www.nature.co |
| | GDEADYYCGAWDSSLSAGGVYVFGT | | | | | | | | | m/articles/s41586-020- |
| | GTKVTVL | | | | | | | | | 2456-9) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| C118 | Ab | SARS-CoV1, | | SARS-CoV2 | | S; RBD | B-cells; SARS-CoV2 | 165 | QVQLVESGGGVVQPGRSLRLSCAASGF | 1244 |
| | | SARS-CoV2 | | (weak) | | | Human Patient | | TFSNYAMHWVRQAPGKGLEWVAVISY | |
| | | | | | | | | | DGSNKYYADSVKGRFTISRDNSKNTLYL | |
| | | | | | | | | | QMNSLRAEDTAIYYCASGYTGYDYFVRG | |
| | | | | | | | | | DYYGLDVWGQGTTVTVSS | |
| C119 | Ab | SARS-CoV2 | SARS- | SARS-CoV2 | SARS-CoV1 | S; RBD | B-cells; SARS-CoV2 | 166 | QVQLVQSGAEVKKPGASVKVSCKASGY | 1245 |
| | | | CoV1 | | | | Human Patient | | TFTSYYMHWVRQAPGQGLEWMGIINP | |
| | | | | | | | | | SGGSTSYAQKLQGRVTMTRDTSTSTVY | |
| | | | | | | | | | MELSSLRSEDTAVYYCARANHETTMDTY | |
| | | | | | | | | | YYYYYMDVWGKGTTVTVSS | |
| C120 | Ab | SARS-CoV2 | SARS- | SARS-CoV2 | SARS-CoV1 | S; RBD | B-cells; SARS-CoV2 | 167 | EVQLVESGGGLIQPGGSLRLSCAASGFT | 1246 |
| | | | CoV1 | | | | Human Patient | | VSSNYMTWVRQAPGKGLEWVSLIYPG | |
| | | | | | | | | | GSTYYADSVKGRFTISRDNSKNTLYLQM | |
| | | | | | | | | | NSLRAEDTAVYYCAREGMGMAAAGTW | |
| | | | | | | | | | GQGTLVTVSS | |
| C121 | Ab | SARS-CoV2 | SARS- | SARS-CoV2 | SARS-CoV1 | S; RBD | B-cells; SARS-CoV2 | 168 | QVQLVQSGAEVKKPGASVKVSCKASGY | 1247 |
| | | | CoV1 | | | | Human Patient | | TFTGYYMHWVRQAPGQGLEWMGWIS | |
| | | | | | | | | | PVSGGTNYAQKFQGRVTMTRDTSISTAY | |
| | | | | | | | | | MELSRLRSDDTAVYYCARAPLFPTGVLA | |
| | | | | | | | | | GDYYYYGMDVWGQGTTVTVSS | |
| C122 | Ab | SARS-CoV2 | SARS- | SARS-CoV2 | SARS-CoV1 | S; RBD | B-cells; SARS-CoV2 | 169 | EVQLVESGGGLIQPGGSLRLSCAASGLT | 1248 |
| | | | CoV1 | | | | Human Patient | | VSSNYMSWVRQAPGKGLEWVSVLYSG | |
| | | | | | | | | | GSSFYADSVKGRFTISRDNSKNTLYLQM | |
| | | | | | | | | | NSLRAEDTAVYYCARESGDTTMAFDYW | |
| | | | | | | | | | GQGTLVTVSS | |
| C123 | Ab | SARS-CoV2 | SARS- | SARS-CoV2 | SARS-CoV1 | S; RBD | B-cells; SARS-CoV2 | 170 | EVQLVESGGGLIQPGGSLRLSCAASGVT | 1249 |
| | | | CoV1 | | | | Human Patient | | VSRNYMSWVRQAPGKGLEWVSVIYSG | |
| | | | | | | | | | GSTYYADSVKGRFTISRDNSKNTLYLQM | |
| | | | | | | | | | NSLRAEDTAVYYCARDLSAAFDIWGQG | |
| | | | | | | | | | TMVTVSS | |
| C124 | Ab | SARS-CoV2 | SARS- | SARS-CoV2 | SARS-CoV1 | S; RBD | B-cells; SARS-CoV2 | 171 | EVQLVESGGGLVQPGGSLRLSCAASGFT | 1250 |
| | | | CoV1 | (weak) | | | Human Patient | | FSGYSMNWVRQAPGKGPEWVSYISRSS | |
| | | | | | | | | | STIYYADSVKGRFTISRDNAKNSLYLQMN | |
| | | | | | | | | | SLRDEDTAVYYCAREGARVGATYDTYYF | |
| | | | | | | | | | DYWGQGTLVTVSS | |
| C125 | Ab | SARS-CoV2 | SARS- | SARS-CoV2 | SARS-CoV1 | S; RBD | B-cells; SARS-CoV2 | 172 | QVQLVQSGPEVKKPGTSVKVSCKASGFT | 1251 |
| | | | CoV1 | | | | Human Patient | | FTSSAVQWVRQARGQRLEWIGWIVVG | |
| | | | | | | | | | SGNTNYAQKFQERVTITRDMSTSTAYM | |
| | | | | | | | | | ELSSLRSEDTAVYYCAAPYCSGGSCSDAF | |
| | | | | | | | | | DIWGQGTMVTVSS | |
| C126 | Ab | SARS-CoV2 | SARS- | | SARS-CoV1, | S; RBD | B-cells; SARS-CoV2 | 173 | QVQLQESGPGLVKPSETLSLSCAVSGGSI | 1252 |
| | | | CoV1 | | SARS-CoV2 | | Human Patient | | GSYFWSWIRQPPGKGLEWIGYLHYSGS | |
| | | | | | | | | | TNYNPSLKSRVTISVDTSKNQFSLKLSSVT | |
| | | | | | | | | | AADTAVYYCARLQWLRGAFDIWGQGT | |
| | | | | | | | | | MVTVSS | |
| C127 | Ab | SARS-CoV2 | SARS- | SARS-CoV2 | SARS-CoV1 | S; RBD | B-cells; SARS-CoV2 | 174 | QVQLVQSGAEVKKPGASVKVSCKASGY | 1253 |
| | | | CoV1 | | | | Human Patient | | TFTGYYMHWVRQAPGQGLEWMGWI | |
| | | | | | | | | | NPNSGGTNYAQKFQGRVTMTRDTSIST | |
| | | | | | | | | | AYMELSRLRSDDTAVYYCATAHPRRIQG | |
| | | | | | | | | | VFFLGPGVWGQGTTVTVSS | |
| C128 | Ab | SARS-CoV2 | SARS- | SARS-CoV2 | SARS-CoV1 | S; RBD | B-cells; SARS-CoV2 | 175 | EVQLLESGGGLVQPGGSLRLSCAASGFT | 1254 |
| | | | CoV1 | | | | Human Patient | | FSTYAMSWVRQAPGKGLEWVSTITGSG | |
| | | | | | | | | | RDTYYADSVKGRFTISRDNSKNTLFLQLN | |
| | | | | | | | | | SLRAEDAAVYSCANHPLASGDDYYHYY | |
| | | | | | | | | | MDVWGKGTTVTVSS | |
| C130 | Ab | SARS-CoV2 | SARS- | | SARS-CoV1, | S; RBD | B-cells; SARS-CoV2 | 176 | QVQLVQSGAEVKKPGASVKVSCKASGY | 1255 |
| | | | CoV1 | | SARS-CoV2 | | Human Patient | | TFTNYYMHWVRQAPGQGLEWMGIINP | |
| | | | | | | | | | SGGSTGYAQKFQGRVTMTRDTSTSTVY | |
| | | | | | | | | | MELSSLRSEDTAVYYCARSRPTPDWYFD | |
| | | | | | | | | | LWGRGTLVTVSS | |
| C131 | Ab | SARS-CoV2 | SARS- | SARS-CoV2 | SARS-CoV1 | S; RBD | B-cells; SARS-CoV2 | 177 | QVQLVQSGSEVKKPGSSVKVSCKASGG | 1256 |
| | | | CoV1 | | | | Human Patient | | TFSSYAFSWVRQAPGQGLEWMGRIIPIL | |
| | | | | | | | | | ALANYAQKFQGRVTITADKSTSTAYMEL | |
| | | | | | | | | | SSLRSEDTAVYYCARVNQAVTTPFSMDV | |
| | | | | | | | | | WGQGTTVTVSS | |
| C132 | Ab | SARS-CoV2 | SARS- | SARS-CoV2 | SARS-CoV1 | S; RBD | B-cells; SARS-CoV2 | 178 | QVQLQESGPGLVKPSGTLSLTCAVSGGS | 1257 |
| | | | CoV1 | (weak) | | | Human Patient | | ISSNNWWSCVRQPPGKGLEWIGEIYHS | |
| | | | | | | | | | GSTNYNPSLKSRVTISVDKSKNQFSLKLSS | |
| | | | | | | | | | VTAADTAVYYCARGGDTAMGPEYFDY | |
| | | | | | | | | | WGQGTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| C118 | QPVLTQSPSASASLGASVKLTCTLSSGH | IGHV3-30 | IGHJ6 | IGLV4-69 | IGLJ3 | 2253 | ASGYTGYDYF | 3455 | QTWGTGIL | Davide Robbiani et al., |
| | SSYAIAWHQQQPEKGPRYLMKLNTD | (Human) | (Human) | (Human) | (Human) | | VRGDYYGLD | | V | 2020 |
| | GSHSKGDGIPDRFSGSSSGAERYLTISS | | | | | | V | | | (https://www.nature.co |
| | LQSEDEADYYCQTWGTGILVFGGGTK | | | | | | | | | m/articles/s41586-020- |
| | LTVL | | | | | | | | | 2456-9) |
| C119 | QSALTQPASVSGSPGQSITISCTGTSSD | IGHV1-46 | IGHJ6 | IGLV2-14 | IGLJ3 | 2254 | ARANHETTM | 3456 | SSYTSSST | Davide Robbiani et al., |
| | VGGYKYVSWYQRHPGKAPKLMIYDV | (Human) | (Human) | (Human) | (Human) | | DTYYYYYYMD | | SVV | 2020 |
| | SNRPSGVSNRFSGSKSGNTASLTISGL | | | | | | V | | | (https://www.nature.co |
| | QAEDEADYYCSSYTSSSTSVVFGGGTQ | | | | | | | | | m/articles/s41586-020- |
| | LTVL | | | | | | | | | 2456-9) |
| C120 | AIRMITQSPSSLSASVGDTVTITCQASQ | IGHV3-53 | IGHJ4 | IGKV1-33 | IGKJ4 | 2255 | AREGMGMA | 3457 | QQYDNLP | Davide Robbiani et al., |
| | DISKYLNWYQQKPGKAPKLLIYDASNL | (Human) | (Human) | (Human) | (Human) | | AAGT | | QT | 2020 |
| | ETGVPSRFSGSGSGTDFTFTISSLQPED | | | | | | | | | (https://www.nature.co |
| | IATYYCQQYDNLPQTFGGGTKVEIK | | | | | | | | | m/articles/s41586-020- |
| | | | | | | | | | | 2456-9) |
| C121 | QSALTQPASVSGSPGQSITISCTGTSSD | IGHV1-2 | IGHJ6 | IGLV2-23 | IGLJ3 | 2256 | ARAPLFPTGV | 3458 | CSYAGSST | Davide Robbiani et al., |
| | VGSYNLVSWYQQHPGKAPKLMIYEGS | (Human) | (Human) | (Human) | (Human) | | LAGDYYYYG | | LV | 2020 |
| | KRPSGVSNRFSGSKSGNTASLTISGLQ | | | | | | MDV | | | (https://www.nature.co |
| | AEDEADYYCCSYAGSSTLVFGGGTKLT | | | | | | | | | m/articles/s41586-020- |
| | VL | | | | | | | | | 2456-9) |
| C122 | DIQLTQSPSFLSASVGDRVTITCRASQG | IGHV3-53 | IGHJ4 | IGKV1-9 | IGKJ2 | 2257 | ARESGDTTM | 3459 | QQLNSDSY | Davide Robbiani et al., |
| | ISSYLAWYQQKPGKAPKLLIYAASTLQS | (Human) | (Human) | (Human) | (Human) | | AFDY | | T | 2020 |
| | GVPSRFSGSGSGTEFTLTISSLQPEDFA | | | | | | | | | (https://www.nature.co |
| | TYYCQQLNSDSYTFGQGTKLEIK | | | | | | | | | m/articles/s41586-020- |
| | | | | | | | | | | 2456-9) |
| C123 | DIQLTQSPSFLSASVGDRVTITCRASQG | IGHV3-53 | IGHJ3 | IGKV1-9 | IGKJ5 | 2258 | ARDLSAAFDI | 3460 | QQLNSYPP | Davide Robbiani et al., |
| | ISSYLAWYQQKPGKAPKLLIYAASTLQS | (Human) | (Human) | (Human) | (Human) | | | | A | 2020 |
| | GVPSRFSGSGSGTEFTLTISSLQPEDFA | | | | | | | | | (https://www.nature.co |
| | TYYCQQLNSYPPAFGQGTRLEIK | | | | | | | | | m/articles/s41586-020- |
| | | | | | | | | | | 2456-9) |
| C124 | EIVLTQSPATLSLSPGERATLSCRASQSF | IGHV3-48 | IGHJ4 | IGKV3-11 | IGKJ1 | 2259 | AREGARVGA | 3461 | QQRNNW | Davide Robbiani et al. |
| | SSYLAWYQQKPGQAPRLLIYDASNRA | (Human) | (Human) | (Human) | (Human) | | TYDTYYFDY | | PPEWT | 2020 |
| | TGIPARFSGSGSGTDFTLTISSLEPEDFA | | | | | | | | | (https://www.nature.co |
| | VYYCQQRNNWPPEWTFGQGTKVEIK | | | | | | | | | m/articles/s41586-020- |
| | | | | | | | | | | 2456-9) |
| C125 | EIVLTQSPGTLSLSPGERATLSCRASQS | IGHV1-58 | IGHJ3 | IGKV3-20 | IGKJ1 | 2260 | AAPYCSGGSC | 3462 | QQYGSSP | Davide Robbiani et al. |
| | VSSSYLAWYQQKPGQAPRLLIYGASSR | (Human) | (Human) | (Human) | (Human) | | SDAFDI | | WT | 2020 |
| | ATGIPDRFSGSGSGTDFTLTISRLEPED | | | | | | | | | (https://www.nature.co |
| | FAVYYCQQYGSSPWTFGQGTKVEIK | | | | | | | | | m/articles/s41586-020- |
| | | | | | | | | | | 2456-9) |
| C126 | NFMLTQPHSVSESPGKTVTISCTGSSG | IGHV4-59 | IGHJ3 | IGLV6-57 | IGLJ3 | 2261 | ARLQWLRGA | 3463 | QSYDSSNL | Davide Robbiani et al., |
| | SIASNYVQWYQQRPGSAPTTVINEDN | (Human) | (Human) | (Human) | (Human) | | FDI | | V | 2020 |
| | QRPSGVPDRFSGSIDSSSNSASLTISGL | | | | | | | | | (https://www.nature.co |
| | KTEDEADYYCQSYDSSNLVFGGGTKLT | | | | | | | | | m/articles/s41586-020- |
| | VL | | | | | | | | | 2456-9) |
| C127 | QSVLTQPPSASGTPGQRVTISCSGSSS | IGHV1-2 | IGHJ6 | IGLV1-44 | IGLJ3 | 2262 | ATAHPRRIQG | 3464 | AAWDDSL | Davide Robbiani et al., |
| | NIGSNTVNWYQQLPGTAPKLLIYSNN | (Human) | (Human) | (Human) | (Human) | | VFFLGPGV | | NGVV | 2020 |
| | QRPSGVPDRFSGSKSGTSASLAISGLQ | | | | | | | | | (https://www.nature.co |
| | SEDEADYYCAAWDDSLNGVVFGGGT | | | | | | | | | m/articles/s41586-020- |
| | KLTVL | | | | | | | | | 2456-9) |
| C128 | EIVLTQSPGTLSLSPGERATLSCRASQS | IGHV3-23 | IGHJ6 | IGKV3-20 | IGKJ4 | 2263 | ANHPLASGD | 3465 | QQYGSSRA | Davide Robbiani et al., |
| | VNSRQLAWYQQKPGQAPRLLIYGASS | (Human) | (Human) | (Human) | (Human) | | DYYHYYMDV | | LT | 2020 |
| | RATGIPERFSGSGSGTDFTLTISRLESED | | | | | | | | | (https://www.nature.co |
| | FAVYHCQQYGSSRALTFGGGTKVEIK | | | | | | | | | m/articles/s41586-020- |
| | | | | | | | | | | 2456-9) |
| C130 | SYELTQPPSVSVAPGKTARITCGGNNI | IGHV1-46 | IGHJ2 | IGLV3-21 | IGLJ3 | 2264 | ARSRPTPDW | 3466 | QVWDSSS | Davide Robbiani et al. |
| | GSKSVHWYQQKPGQAPVLVIYYDSDR | (Human) | (Human) | (Human) | (Human) | | YFDL | | DHPGVV | 2020 |
| | PSGIPERFSGSNSGNTATLTISRVEAGD | | | | | | | | | (https://www.nature.co |
| | EADYYCQVWDSSSDHPGVVFGGGTK | | | | | | | | | m/articles/s41586-020- |
| | LTVL | | | | | | | | | 2456-9) |
| C131 | EIVMTQSPATLSVSPGERATLSCRASQ | IGHV1-69 | IGHJ6 | IGKV3-15 | IGKJ5 | 2265 | ARVNQAVTT | 3467 | QQYNNWP | Davide Robbiani et al., |
| | SVSSNLAWYQQKPGQAPRLLIYGAST | (Human) | (Human) | (Human) | (Human) | | PFSMDV | | IT | 2020 |
| | RATGIPARFSGSGSGTEFTLTISSLQSE | | | | | | | | | (https://www.nature.co |
| | DFAVYYCQQYNNWPITFGQGTRLEIK | | | | | | | | | m/articles/s41586-020- |
| | | | | | | | | | | 2456-9) |
| C132 | QSALTQPASVSGSPGQSITISCTGTSSD | IGHV4-4 | IGHJ4 | IGLV2-14 | IGLJ3 | 2266 | ARGGDTAMG | 3468 | SSYTSSST | Davide Robbiani et al. |
| | VGGYNYVSWYQQHPGKAPKLMIYDV | (Human) | (Human) | (Human) | (Human) | | PEYFDY | | LL | 2020 |
| | SNRPSGVSNRFSGSKSGNTASLTISGL | | | | | | | | | (https://www.nature.co |
| | QAEDEADYYCSSYTSSSTLLFGGGTKLT | | | | | | | | | m/articles/s41586-020- |
| | VL | | | | | | | | | 2456-9) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| C133 | Ab | SARS-CoV2 | SARS- | | SARS-CoV1, | S; RBD | B-cells; SARS-CoV2 | 179 | QVQLVESGGGVVQPGRSLRLSCAASGF | 1258 |
| | | | CoV1 | | SARS-CoV2 | | Human Patient | | TFSSYAMHWVRQAPGKGLEWVAVILY | |
| | | | | | | | | | DGSNKYYADSVKGRFTISRDNSKNTLYL | |
| | | | | | | | | | QMNSLRAEDTAVYYCARDSDVDTSMVT | |
| | | | | | | | | | WFDYWGQGTLVTVSS | |
| C134 | Ab | SARS-CoV2 | SARS- | | SARS-CoV1, | S; RBD | B-cells; SARS-CoV2 | 180 | EVQLLESGGGLVQPGGSLRLSCAASGFT | 1259 |
| | | | CoV1 | | SARS-CoV2 | | Human Patient | | FSNYAMSWVRQAPGKGLEWVSAISGS | |
| | | | | | | | | | DGSTYYAGSVKGRFTISRDNSKNTLYLQ | |
| | | | | | | | | | MNSLRAEDTAVYYCAKDPLITGPTYQYF | |
| | | | | | | | | | HYWGQGTLVTVSS | |
| C135 | Ab | SARS-CoV2 | SARS- | SARS-CoV2 | SARS-CoV1 | S; RBD | B-cells; SARS-CoV2 | 181 | QVQLVESGGGVVQPGRSLRLSCAASGF | 1260 |
| | | | CoV1 | | | | Human Patient | | TFSSYAMHWVRQAPGKGLEWVAVIPF | |
| | | | | | | | | | DGRNKYYADSVTGRFTISRDNSKNTLYL | |
| | | | | | | | | | QMNSLRAEDTAVYYCASSSGYLFHSDY | |
| | | | | | | | | | WGQGTLVTVSS | |
| C138 | Ab | SARS-CoV2 | SARS- | | SARS-CoV1, | S; RBD | B-cells; SARS-CoV2 | 182 | EVQLVESGGGLVQPGGSLRLSCAASGFT | 1261 |
| | | | CoV1 | | SARS-CoV2 | | Human Patient | | FSTYWMSWVRQPPGKGLEWVANIKQ | |
| | | | | | | | | | DGSEKYYVDSVKGRFTISRDNAKNSLYL | |
| | | | | | | | | | QMNSLRADDTAVYYCAGGTWLRSSFDY | |
| | | | | | | | | | WGQGTLVTVSS | |
| C139 | Ab | SARS-CoV2 | SARS- | | SARS-CoV1, | S; RBD | B-cells; SARS-CoV2 | 183 | EVQLVESGGGVVQPGRSLRLSCAASGFT | 1262 |
| | | | CoV1 | | SARS-CoV2 | | Human Patient | | FSSYAMHWVRQAPGKGLEWVAVISYD | |
| | | | | | | | | | GSNKYSADSVKGRFTISRDNSKNTLYLQ | |
| | | | | | | | | | MNSLRAEDTAVYYCAKGGAYSYYYYMD | |
| | | | | | | | | | VWGKGTTVTVSS | |
| C140 | Ab | SARS-CoV2 | SARS- | SARS-CoV2 | SARS-CoV1 | S; RBD | B-cells; SARS-CoV2 | 184 | EVQLVESGGGLVQPGGSLRLSCAASGVT | 1263 |
| | | | CoV1 | | | | Human Patient | | VSSNYMSWVRQAPGKGLEWVSLIYSGG | |
| | | | | | | | | | STFYADSVKGRFTISRDNSENTLYLQMN | |
| | | | | | | | | | TLRAEDTAVYYCARDLYYYGMDVWGQ | |
| | | | | | | | | | GTTVTVSS | |
| C141 | Ab | SARS-CoV2 | SARS- | | SARS-CoV1, | S; RBD | B-cells; SARS-CoV2 | 185 | EVQLVESGGGVVQPGRSLRLSCAASGFT | 1264 |
| | | | CoV1 | | SARS-CoV2 | | Human Patient | | FSSYAMFWVRQAPGKGLEWVAVISYD | |
| | | | | | | | | | GSNKYYADSVKGRFTISRDNSKNTLYLQ | |
| | | | | | | | | | MNSLRAEDTAVYYCARADLGYCTNGVC | |
| | | | | | | | | | YVDYWGQGTLVTVSS | |
| C143 | Ab | SARS-CoV2 | SARS- | | SARS-CoV1, | S; RBD | B-cells; SARS-CoV2 | 186 | EVQLVESGGGLVQPGGSLRLSCAASGFS | 1265 |
| | | | CoV1 | | SARS-CoV2 | | Human Patient | | VSTKYMTWVRQAPGKGLEWVSVLYSG | |
| | | | | | | | | | GSDYYADSVKGRFTISRDNSKNALYLQM | |
| | | | | | | | | | NSLRVEDTGVYYCARDSSEVRDHPGHP | |
| | | | | | | | | | GRSVGAFDIWGQGTMVTVSS | |
| C144 | Ab | SARS-CoV2 | SARS- | SARS-CoV2 | SARS-CoV1 | S; RBD | B-cells; SARS-CoV2 | 187 | EVQLVESGGGLIQPGGSLRLSCAASGFT | 1266 |
| | | | CoV1 | | | | Human Patient | | VSNNYMSWVRQAPGKGLEWVSVIYSG | |
| | | | | | | | | | GSTYYADSVKGRFTISRDKSKNTLYLQM | |
| | | | | | | | | | NRLRAEDTAVYYCAREGEVEGYNDFWS | |
| | | | | | | | | | GYSRDRYYFDYWGQGTLVTVSS | |
| C145 | Ab | SARS-CoV2 | SARS- | SARS-CoV2 | SARS-CoV1 | S; RBD | B-cells; SARS-CoV2 | 188 | EVQLVESGGGLIQPGGSLRLSCAASGFS | 1267 |
| | | | CoV1 | | | | Human Patient | | VSSNYMSWVRQAPGKGLEWVSVIYSG | |
| | | | | | | | | | GSTYYADSVKGRFTISRDNSKNTLYLQM | |
| | | | | | | | | | NSLRAEDTAVYYCAREGEVEGYYDFWS | |
| | | | | | | | | | GYSRDRYYFDYWGQGTLVTVSS | |
| C146 | Ab | SARS-CoV2 | SARS- | | SARS-CoV1, | S; non- | B-cells; SARS-CoV2 | 189 | EVQLVESGGGLVKPGGSLRLSCAASGLT | 1268 |
| | | | CoV1 | | SARS-CoV2 | RBD | Human Patient | | FTAYRMNWVRQAPGKGLEWLSSISNTN | |
| | | | | | | | | | GDIYYADSVKGRFTISRDNAKNSLYLQM | |
| | | | | | | | | | NSLRADDTAVYYCARDVASNYAYFDLW | |
| | | | | | | | | | GQGTLVTVSS | |
| C147 | Ab | SARS-CoV2 | SARS- | | SARS-CoV1, | S; non- | B-cells; SARS-CoV2 | 190 | EVQLVQSGAEVKKPGESLKISCKGSGYR | 1269 |
| | | | CoV1 | | SARS-CoV2 | RBD | Human Patient | | FTNYWIGWVRQMPGKGLEWMGIIYPG | |
| | | | | | | | | | DSDTRYSPSFQGQVTISADKSITTAYLQW | |
| | | | | | | | | | SSLKASDTAMYYCARLSDRWYSPFDPW | |
| | | | | | | | | | GQGTLVTVSS | |
| C148 | Ab | SARS-CoV2 | SARS- | | SARS-CoV1, | S; RBD | B-cells; SARS-CoV2 | 191 | EVQLVESGGGLVQPGGSQRLSCAASGF | 1270 |
| | | | CoV1 | | SARS-CoV2 | | Human Patient | | TVSSNYMSWIRQAPGKGLEWVSVIYSG | |
| | | | | | | | | | GSAYYVDSVKGRFTISRDNSKNTLYLQM | |
| | | | | | | | | | NSLRPEDTAVYYCARIANYMDVWGKGT | |
| | | | | | | | | | TVTVSS | |
| C150 | Ab | SARS-CoV2 | SARS- | | SARS-CoV1, | S; RBD | B-cells; SARS-CoV2 | 192 | EVQLVESGGGLVQPGGSLRLSCVASGFT | 1271 |
| | | | CoV1 | | SARS-CoV2 | | Human Patient | | FSSYWMHWVRQVPGKGPVWVSHINS | |
| | | | | | | | | | EGSSTNYADSVRGRFTISRDNAKDTLYL | |
| | | | | | | | | | QMNNLRAEDTAVYYCARPTAVAAAGN | |
| | | | | | | | | | YFYYYGMDVWGQGTTVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| C133 | DIQMTQSPSSLSASVGDRVTITCRASQ | IGHV3-30 | IGHJ4 | IGKV1-39 | IGKJ1 | 2267 | ARDSDVDTS | 3469 | QQSYSTPP | Davide Robbiani et al., |
| | SISSYLNWYQQKPGKAPKLLIYAASSLQ | (Human) | (Human) | (Human) | (Human) | | MVTWFDY | | WT | 2020 |
| | SGVPSRFSGSGSGTDFTLTISSLQPEDF | | | | | | | | | (https://www.nature.co |
| | ATYYCQQSYSTPPWTFGQGTKVEIK | | | | | | | | | m/articles/s41586-020- |
| | | | | | | | | | | 2456-9) |
| C134 | SYELTQPPSVSVAPGKTARITCGGNNI | IGHV3-23 | IGHJ4 | IGLV3-21 | IGLJ3 | 2268 | AKDPLITGPTY | 3470 | QVWDSSS | Davide Robbiani et al., |
| | GSKSVHWYQQKPGQAPVLVIYYDSDR | (Human) | (Human) | (Human) | (Human) | | QYFHY | | DRPGVV | 2020 |
| | PSGIPERFSGSNSGNTATLTISRVEAGD | | | | | | | | | (https://www.nature.co |
| | EAEYHCQVWDSSSDRPGVVFGGGTK | | | | | | | | | m/articles/s41586-020- |
| | LTVL | | | | | | | | | 2456-9) |
| C135 | DIQMTQSPSTLSASVGDRVTITCRASQ | IGHV3-30 | IGHJ4 | IGKV1-5 | IGKJ1 | 2269 | ASSSGYLFHS | 3471 | QQYNSYP | Davide Robbiani et al., |
| | SISNWLAWFQQKPGKAPKLLIYEASSL | (Human) | (Human) | (Human) | (Human) | | DY | | WT | 2020 |
| | ESGVPSRFSGSGSGTEFTLTISSLQPDD | | | | | | | | | (https://www.nature.co |
| | FATYYCQQYNSYPWTFGQGTKVEIK | | | | | | | | | m/articles/s41586-020- |
| | | | | | | | | | | 2456-9) |
| C138 | NFMLTQPHSVSESPGKTVTISCTGSSG | IGHV3-7 | IGHJ4 | IGLV6-57 | IGLJ3 | 2270 | AGGTWLRSSF | 3472 | QSYDSSN | Davide Robbiani et al., |
| | SIASNYVQWYQQRPGSAPTTVIYEDN | (Human) | (Human) | (Human) | (Human) | | DY | | WV | 2020 |
| | QRPSGVPDRFSGSIDSSSNSASLTISGL | | | | | | | | | (https://www.nature.co |
| | KTEDEADYYCQSYDSSNWVFGGGTKL | | | | | | | | | m/articles/s41586-020- |
| | TVL | | | | | | | | | 2456-9) |
| C139 | DIQMTQSPSSLSASVGDRVTITCQASQ | IGHV3-30- | IGHJ6 | IGKV1-33 | IGKJ4 | 2271 | AKGGAYSYYY | 3473 | QQYDNLPL | Davide Robbiani et al., |
| | DISNYLNWYQQKPGKAPKLLIYDASNL | 3 | (Human) | (Human) | (Human) | | YMDV | | T | 2020 |
| | ETGVPSRFSGSGSGTDFTFTISSLQPED | (Human) | | | | | | | | (https://www.nature.co |
| | IATYYCQQYDNLPLTFGGGTKVEIK | | | | | | | | | m/articles/s41586-020- |
| | | | | | | | | | | 2456-9) |
| C140 | DIQLTQSPSFLSASVGDRVTITCRASQG | IGHV3-66 | IGHJ6 | IGKV1-9 | IGKJ2 | 2272 | ARDLYYYGM | 3474 | QQLNSYSY | Davide Robbiani et al., |
| | ISSYLAWYQQKPGKAPKLLIYAASTLQS | (Human) | (Human) | (Human) | (Human) | | DV | | T | 2020 |
| | GVPSRFSGSGSGTEFTLTISSLQPEDFA | | | | | | | | | (https://www.nature.co |
| | TYYCQQLNSYSYTFGQGTKLEIK | | | | | | | | | m/articles/s41586-020- |
| | | | | | | | | | | 2456-9) |
| C141 | NFMLTQPHSVSESPGKTVTISCTGSSG | IGHV3-30 | IGHJ4 | IGLV6-57 | IGLJ3 | 2273 | ARADLGYCTN | 3475 | QSYDSSN | Davide Robbiani et al. |
| | SIASNYVQWYQQRPGSAPTTVIYEDN | (Human) | (Human) | (Human) | (Human) | | GVCYVDY | | WV | 2020 |
| | QRPSGVPDRFSGSIDSSSNSASLTISGL | | | | | | | | | (https://www.nature.co |
| | KTEDEADYYCQSYDSSNWVFGGGTKL | | | | | | | | | m/articles/s41586-020- |
| | TVL | | | | | | | | | 2456-9) |
| C143 | QSALTQPASVSGSPGQSITISCTGTSND | IGHV3-66 | IGHJ3 | IGLV2-23 | IGLJ3 | 2274 | ARDSSEVRDH | 3476 | CSYAGAST | Davide Robbiani et al., |
| | VGSYTLVSWYQQYPGKAPKLLIFEGTK | (Human) | (Human) | (Human) | (Human) | | PGHPGRSVG | | FV | 2020 |
| | RSSGISNRFSGSKSGNTASLTISGLQGE | | | | | | AFDI | | | (https://www.nature.co |
| | DEADYYCCSYAGASTFVFGGGTKLTVL | | | | | | | | | m/articles/s41586-020- |
| | | | | | | | | | | 2456-9) |
| C144 | QSALTQPASVSGSPGQSITISCTGTSSD | IGHV3-53 | IGHJ4 | IGLV2-14 | IGLJ1 | 2275 | AREGEVEGY | 3477 | SSYTSSST | Davide Robbiani et al., |
| | VGGYNYVSWYQQHPGKAPKLMIYDV | (Human) | (Human) | (Human) | (Human) | | NDFWSGYSR | | RV | 2020 |
| | SNRPSGVSNRFSGSKSGNTASLTISGL | | | | | | DRYYFDY | | | (https://www.nature.co |
| | QAEDEADYYCSSYTSSSTRVFGTGTKV | | | | | | | | | m/articles/s41586-020- |
| | TVL | | | | | | | | | 2456-9) |
| C145 | QSALTQPASVSGSPGQSITISCTGTSSD | IGHV3-53 | IGHJ4 | IGLV2-14 | IGLJ1 | 2276 | AREGEVEGYY | 3478 | SSYTSSTT | Davide Robbiani et al., |
| | VGGYNYVSWYQQHPGKAPKLMIYDV | (Human) | (Human) | (Human) | (Human) | | DFWSGYSRD | | RV | 2020 |
| | SNRPSGVSNRFSGSKSGNTASLTISGL | | | | | | RYYFDY | | | (https://www.nature.co |
| | QAEDEADYYCSSYTSSTTRVFGTGTRV | | | | | | | | | m/articles/s41586-020- |
| | TVL | | | | | | | | | 2456-9) |
| C146 | QSALTQPASVSGSPGQSITISCTGTSSD | IGHV3-21 | IGHJ4 | IGLV2-14 | IGLJ1 | 2277 | ARDVASNYAY | 3479 | SSYRGSST | Davide Robbiani et al., |
| | IGVYNYISWSQQHPGKAPKVMIYDVT | (Human) | (Human) | (Human) | (Human) | | FDL | | PYV | 2020 |
| | NRPSGVSNRFSGSKSGNTASLTISGLQ | | | | | | | | | (https://www.nature.co |
| | AEDEADYYCSSYRGSSTPYVFGTGTKV | | | | | | | | | m/articles/s41586-020- |
| | TVL | | | | | | | | | 2456-9) |
| C147 | QAVVTQEPSLTVSPGGTVTLTCGSSTG | IGHV5-51 | IGHJ5 | IGLV7-46 | IGLJ3 | 2278 | ARLSDRWYSP | 3480 | LLSYSGAR | Davide Robbiani et al., |
| | AVTSGHYPYWFQQKSGQAPRTLIYETS | (Human) | (Human) | (Human) | (Human) | | FDP | | PV | 2020 |
| | IKHSWTPARFSGSLLGGKAALTLSGAQ | | | | | | | | | (https://www.nature.co |
| | PEDEADYYCLLSYSGARPVFGGGTKLT | | | | | | | | | m/articles/s41586-020- |
| | VL | | | | | | | | | 2456-9) |
| C148 | EIVMTQSPATLSVSPGERATLSCRASQ | IGHV3-66 | IGHJ6 | IGKV3-15 | IGKJ4 | 2279 | ARIANYMDV | 3481 | QQYNNWP | Davide Robbiani et al., |
| | SVSSHLAWYQQKPGQAPRLLIYGAST | (Human) | (Human) | (Human) | (Human) | | | | PLT | 2020 |
| | RATGIPTRFSGSGSGTEFTLTISSLQSED | | | | | | | | | (https://www.nature.co |
| | FAVYYCQQYNNWPPLTFGGGTKVEIK | | | | | | | | | m/articles/s41586-020- |
| | | | | | | | | | | 2456-9) |
| C150 | QSALTQPASVSGSPGQSITISCTGTSSD | IGHV3-74 | IGHJ6 | IGLV2-14 | IGLJ3 | 2280 | ARPTAVAAA | 3482 | SSYRSSST | Davide Robbiani et al., |
| | VGYYNFVSWYQQHPGKAPKLMIYEVS | (Human) | (Human) | (Human) | (Human) | | GNYFYYYGM | | LV | 2020 |
| | NRPSGVSNRFSGSKSGNTASLIISGLQA | | | | | | DV | | | (https://www.nature.co |
| | EDEADYYCSSYRSSSTLVFGGGTKLTVL | | | | | | | | | m/articles/s41586-020- |
| | | | | | | | | | | 2456-9) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| C151 | Ab | SARS-CoV2 | SARS- | SARS-CoV2 | SARS-CoV1 | S; RBD | B-cells; SARS-CoV2 | 193 | EVQLVESGGGLVKPGGSLRLSCAASGFT | 1272 |
| | | | CoV1 | (weak) | | | Human Patient | | FSSYNMNWVRQAPGKGLEWVSCISSSS | |
| | | | | | | | | | SYIYYADSVKGRFTISRDNAKNSLYLQMN | |
| | | | | | | | | | SLRAEDTAVYYCARERGYDGGKTPPFLG | |
| | | | | | | | | | GQGTLVTVSS | |
| C153 | Ab | SARS-CoV2 | SARS- | SARS-CoV2 | SARS-CoV1 | S; RBD | B-cells; SARS-CoV2 | 194 | EVQLVESGGGLIQPGGSLRLSCAASGFT | 1273 |
| | | | CoV1 | (weak) | | | Human Patient | | VSSNYMSWVRQAPGKGLEWVSVIYSGY | |
| | | | | | | | | | STYYVDSVKGRFTISRDNSKNTLYLQMN | |
| | | | | | | | | | SLRAEDTAVYYCARVGGAHSGYDGSFDY | |
| | | | | | | | | | WGQGTLVTVSS | |
| C154 | Ab | SARS-CoV1, | | SARS-CoV2 | | S; RBD | B-cells; SARS-CoV2 | 195 | QVQLVESGGGVVQPGRSLRLSCAASGF | 1274 |
| | | SARS-CoV2 | | (weak) | | | Human Patient | | TFSRYGMHWVRQAPGKGLEWVAVMS | |
| | | | | | | | | | YDGSSKYYADSVKGRFTISRDNSKNTLCL | |
| | | | | | | | | | QMNSLRAEDTAVYYCAKQAGPYCSGGS | |
| | | | | | | | | | CYSAPFDYWGQGTLVTVSS | |
| C155 | Ab | SARS-CoV2 | SARS- | SARS-CoV2 | SARS-CoV1 | S; RBD | B-cells; SARS-CoV2 | 196 | EVQLVESGGGLIQPGGSLRLSCAASGFIV | 1275 |
| | | | CoV1 | | | | Human Patient | | SSNYMSWVRQAPGKGLEWVSVIYSGG | |
| | | | | | | | | | STFYADSVKGRFTISRDNSKNTLYLQMN | |
| | | | | | | | | | SLRAEDTAVYYCARDFGEFYFDYWGQG | |
| | | | | | | | | | TLVTVSS | |
| C156 | Ab | SARS-CoV2 | SARS- | | SARS-CoV1, | S; RBD | B-cells; SARS-CoV2 | 197 | QVQLVESGGGVVQPGRSLRLSCAASGF | 1276 |
| | | | CoV1 | | SARS-CoV2 | | Human Patient | | TFSNYGMHWVRQAPGKGLEWVAVISY | |
| | | | | | | | | | DGNNKYYADSVKGRFTISRDNSKNTLYL | |
| | | | | | | | | | QMNSLRAEDTAVYYCAKDPFPLAVAGT | |
| | | | | | | | | | GYFDYWGQGTLVTVSS | |
| C164 | Ab | SARS-CoV2 | SARS- | SARS-CoV2 | SARS-CoV1 | S; RBD | B-cells; SARS-CoV2 | 198 | EVQLVESGGGLVQPGGSLRLSCAASGFS | 1277 |
| | | | CoV1 | (weak) | | | Human Patient | | VSTKYMTWVRQAPGKGLEWVSVLYSG | |
| | | | | | | | | | GSDYYADSVKGRFTISRDNSKNALYLQM | |
| | | | | | | | | | NSLRVEDTGVYYCARDSSEVRDHPGHP | |
| | | | | | | | | | GRSVGAFDIWGQGTMVTVSS | |
| C165 | Ab | SARS-CoV2 | SARS- | SARS-CoV2 | SARS-CoV1 | S; RBD | B-cells; SARS-CoV2 | 199 | QVQLVQSGAEVKKPGSSVKVSCKASGG | 1278 |
| | | | CoV1 | | | | Human Patient | | TFSSYAINWVRQAPGQGLEWMGRIIPI | |
| | | | | | | | | | VGIANYAQKFQGRVTITADKSSSTAYME | |
| | | | | | | | | | LSSLRSEDTAVYYCARDLLDPQLDDAFDI | |
| | | | | | | | | | WGQGTMVTVSS | |
| C201 | Ab | SARS-CoV2 | SARS- | | SARS-CoV1, | S; RBD | B-cells; SARS-CoV2 | 200 | EVQLVESGGGLVQPGRSLRLSCAASGFT | 1279 |
| | | | CoV1 | | SARS-CoV2 | | Human Patient | | FDDYAMHWVRQAPGKGLEWVSGISW | |
| | | | | | | | | | NSGSIGYADSVKGRFTISRDNAKNSLYLQ | |
| | | | | | | | | | MNSLRAEDTALYYCVKGVEYSSSSNFDY | |
| | | | | | | | | | WGQGTLVTVSS | |
| C202 | Ab | SARS-CoV2 | SARS- | | SARS-CoV1, | S; RBD | B-cells; SARS-CoV2 | 201 | EVQLVESGGGLVQPGGSLRLSCAASGFT | 1280 |
| | | | CoV1 | | SARS-CoV2 | | Human Patient | | VSSNYMSWVRQAPGKGLEWVSLIYSGG | |
| | | | | | | | | | STYYADSVKGRFTISRDNSKNTLYLQMN | |
| | | | | | | | | | SLRAEDTAVYYCARDTLGRGGDYWGQ | |
| | | | | | | | | | GTLVTVSS | |
| C204 | Ab | SARS-CoV1, | | | SARS-CoV2 | S; RBD | B-cells; SARS-CoV2 | 202 | EVQLLESGGGLEQPGGSLRLSCAASGFT | 1281 |
| | | SARS-CoV2 | | | | | Human Patient | | FSTYAMSWVRQAPGKGLEWVSAISGSG | |
| | | | | | | | | | AGTFYADSVKGRFTISRDNSKNTLYLQM | |
| | | | | | | | | | NSLRAEDTAVYYCARESDCGSTSCYQVG | |
| | | | | | | | | | WFDPWGQGTLVTVSS | |
| C205 | Ab | SARS-CoV2 | SARS- | | SARS-CoV1, | S; non- | B-cells; SARS-CoV2 | 203 | QVQLVQSGAEVKKPGASVKVSCKASGH | 1282 |
| | | | CoV1 | | SARS-CoV2 | RBD | Human Patient | | TFTSYYMHWVRQAPGQGLEWMGIINP | |
| | | | | | | | | | SGGSTSYAQKFQGRVTMTRDTSTSTVY | |
| | | | | | | | | | MELSSLRSEDTAVYYCARGPERGIVGAT | |
| | | | | | | | | | DYFDYWGQGTLVTVSS | |
| C207 | Ab | SARS-CoV2 | SARS- | SARS-CoV2 | SARS-CoV1 | S; RBD | B-cells; SARS-CoV2 | 204 | EVQLLESGGGLVQPGGSLRLSCAASGFT | 1283 |
| | | | CoV1 | (weak) | | | Human Patient | | FSSYAMSWVRQAPGKGLEWVSAISGSG | |
| | | | | | | | | | GSTYYADSVKGRFTISRDNSKNTLYLQM | |
| | | | | | | | | | NSLRAEDTAVYYCAKEPIGQPLLWWDY | |
| | | | | | | | | | WGQGTLVTVSS | |
| C208 | Ab | SARS-CoV2 | SARS- | | SARS-CoV1, | S; non- | B-cells; SARS-CoV2 | 205 | EVQLVQSGAEVKKPGESLKISCKGSGYSF | 1284 |
| | | | CoV1 | | SARS-CoV2 | RBD | Human Patient | | TSYWIGWVRQMPGKGLEWMGIIYPGD | |
| | | | | | | | | | SDTRYSPSFQGQVTISADKSISTAYLKWS | |
| | | | | | | | | | SLKASDSAMYYCARGPNLQNWFDPWG | |
| | | | | | | | | | QGTLVTVSS | |
| C210 | Ab | SARS-CoV2 | SARS- | SARS-CoV2 | SARS-CoV1 | S; RBD | B-cells; SARS-CoV2 | 206 | EVQLVESGGGLIQPGGSLRLSCAASGFT | 1285 |
| | | | CoV1 | | | | Human Patient | | VSSNYMSWVRQAPGKGLEWVSVIYSG | |
| | | | | | | | | | GSTFYADSVKGRFTFSRDNSKNTLYLQM | |
| | | | | | | | | | NSLRAEDTAVYYCARDLMAYGMDVWG | |
| | | | | | | | | | QGTTVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| C151 | NFMLTQPHSVSESPGKTVTISCTGSSG | IGHV3-21 | IGHJ4 | IGLV6-57 | IGLJ3 | 2281 | ARERGYDGG | 3483 | QSYDSSNY | Davide Robbiani et al., |
| | SIASNYVQWYQQRPGSAPTTVIYEDN | (Human) | (Human) | (Human) | (Human) | | KTPP | | WV | 2020 |
| | QRPSGVPDRFSGSIDSSSNSASLTISGL | | | | | | | | | (https://www.nature.co |
| | KTEDEADYYCQSYDSSNYWVFGGGTK | | | | | | | | | m/articles/s41586-020- |
| | LTVL | | | | | | | | | 2456-9) |
| C153 | QSALTQPASVSGSPGQSITISCTGTSSD | IGHV3-53 | IGHJ4 | IGLV2-23 | IGLJ3 | 2282 | ARVGGAHSG | 3484 | CSYAGSST | Davide Robbiani et al., |
| | VGSYNLVSWYQQHPGKAPKLMIYEGS | (Human) | (Human) | (Human) | (Human) | | YDGSFDY | | WV | 2020 |
| | KRPSGVSNRFSGSKSGNTASLTISGLQ | | | | | | | | | (https://www.nature.co |
| | AEDEADYYCCSYAGSSTWVFGGGTKL | | | | | | | | | m/articles/s41586-020- |
| | TVL | | | | | | | | | 2456-9) |
| C154 | DIQMTQSPSSLSASVGDRVTITCQASQ | IGHV3-30 | IGHJ4 | IGKV1-33 | IGKJ5 | 2283 | AKQAGPYCS | 3485 | QQYDNLPI | Davide Robbiani et al., |
| | GISNYLNWYQQKPGKAPKLLIYDASNL | (Human) | (Human) | (Human) | (Human) | | GGSCYSAPFD | | T | 2020 |
| | ETGVPSRFSGSGSGTDFTFTISSLQPED | | | | | | Y | | | (https://www.nature.co |
| | IATYYCQQYDNLPITFGQGTRLEIK | | | | | | | | | m/articles/s41586-020- |
| | | | | | | | | | | 2456-9) |
| C155 | EIVMTQSPATLSVSPGERATLSCRASQ | IGHV3-53 | IGHJ4 | IGKV3-15 | IGKJ1 | 2284 | ARDFGEFYFD | 3486 | QQYNNWP | Davide Robbiani et al., |
| | SVSSNLAWYQQKPGQAPRLLIYGAST | (Human) | (Human) | (Human) | (Human) | | Y | | RT | 2020 |
| | RATAIPARFSGSGSGTEFTLTISSLQSED | | | | | | | | | (https://www.nature.co |
| | FAVYYCQQYNNWPRTFGQGTKVEIK | | | | | | | | | m/articles/s41586-020- |
| | | | | | | | | | | 2456-9) |
| C156 | SYELTQPPSVSVAPGQTARISCGGNNI | IGHV3-30 | IGHJ4 | IGLV3-21 | IGLJ3 | 2285 | AKDPFPLAVA | 3487 | QVWDSSS | Davide Robbiani et al., |
| | GSKNVHWYQQKPGQAPVLVVYDDS | (Human) | (Human) | (Human) | (Human) | | GTGYFDY | | DPWV | 2020 |
| | DRPSGIPERFSGSNSGNTATLTISRVEA | | | | | | | | | (https://www.nature.co |
| | GDEADYYCQVWDSSSDPWVFGGGTK | | | | | | | | | m/articles/s41586-020- |
| | LTVL | | | | | | | | | 2456-9) |
| C164 | QSALTQPASVSGSPGQSITISCTGTSND | IGHV3-66 | IGHJ3 | IGLV2-23 | IGLJ3 | 2286 | ARDSSEVRDH | 3488 | CSYAGAST | Davide Robbiani et al., |
| | VGSYTLVSWYQQYPGKAPKLLIFEVTK | (Human) | (Human) | (Human) | (Human) | | PGHPGRSVG | | FV | 2020 |
| | RSSGISNRFSGSKSGNTASLTISGLQGE | | | | | | AFDI | | | (https://www.nature.co |
| | DEADYYCCSYAGASTFVFGGGTKLTVL | | | | | | | | | m/articles/s41586-020- |
| | | | | | | | | | | 2456-9) |
| C165 | EIVLTQSPGTLSLSPGERATLSCRASQS | IGHV1-69 | IGHJ3 | IGKV3-20 | IGKJ1 | 2287 | ARDLLDPQLD | 3489 | QQYGSSP | Davide Robbiani et al., |
| | VSSTYLAWYQQKPGQAPRLLIYGASSR | (Human) | (Human) | (Human) | (Human) | | DAFDI | | WT | 2020 |
| | ATGIPDRFSGSGSGTDFTLTISRLEPED | | | | | | | | | (https://www.biorxiv.or |
| | FAVYYCQQYGSSPWTFGQGTKVEIK | | | | | | | | | g/content/10.1101/202 |
| | | | | | | | | | | 0.05.13.092619v2) |
| C201 | IRMTQSPSSVSASVGDRVTITCRASQG | IGHV3-9 | IGHJ4 | IGKV1-12 | IGKJ4 | 2288 | VKGVEYSSSS | 3490 | QQANSFPL | Davide Robbiani et al., |
| | ISSWLAWYQQKPGKAPKLLIYVESSLQ | (Human) | (Human) | (Human) | (Human) | | NFDY | | T | 2020 |
| | SGVPSRFSGSGSGTDFTLTISSLQPEDF | | | | | | | | | (https://www.biorxiv.or |
| | ATYYCQQANSFPLTFGGGTKVEIK | | | | | | | | | g/content/10.1101/202 |
| | | | | | | | | | | 0.05.13.092619v2) |
| C202 | DIQLTQSPSSLSASVGDRVTITCQASQ | IGHV3-66 | IGHJ4 | IGKV1-33 | IGKJ2 | 2289 | ARDTLGRGG | 3491 | QQYDNLP | Davide Robbiani et al., |
| | DISNYLNWYQQKPGKAPKLLIYDASNL | (Human) | (Human) | (Human) | (Human) | | DY | | RS | 2020 |
| | ETGVPSRFSGSGSGTDFTFTISSLQPED | | | | | | | | | (https://www.biorxiv.or |
| | IATYYCQQYDNLPRSFGQGTKLEIK | | | | | | | | | g/content/10.1101/202 |
| | | | | | | | | | | 0.05.13.092619v2) |
| C204 | DIQLTQSPSSLSASVGDRVTITCRASQS | IGHV3-23 | IGHJ5 | IGKV1-39 | IGKJ1 | 2290 | ARESDCGSTS | 3492 | QQSYSTPP | Davide Robbiani et al., |
| | ISSYLNWYQQKPGKAPKLLIYAASSLQS | (Human) | (Human) | (Human) | (Human) | | CYQVGWFDP | | WT | 2020 |
| | GVPSRFSGSGSGTDFTLTISSLQPEDFA | | | | | | | | | (https://www.biorxiv.or |
| | TYYCQQSYSTPPWTFGQGTKVEIK | | | | | | | | | g/content/10.1101/202 |
| | | | | | | | | | | 0.05.13.092619v2) |
| C205 | EIVLTQSPGTLSLSPGERATLSCRASQS | IGHV1-46 | IGHJ4 | IGKV3-20 | IGKJ1 | 2291 | ARGPERGIVG | 3493 | QQYVSSP | Davide Robbiani et al., |
| | VSSSYLAWYQQKPGQAPRLLIYGASSR | (Human) | (Human) | (Human) | (Human) | | ATDYFDY | | WT | 2020 |
| | ATGIPDRFSGSGSGTDFTLTISRLEPED | | | | | | | | | (https://www.biorxiv.or |
| | FAVYYCQQYVSSPWTFGQGTKVEIK | | | | | | | | | g/content/10.1101/202 |
| | | | | | | | | | | 0.05.13.092619v2) |
| C207 | EIVLTQSPATLSLSPGERATLSCRASQS | IGHV3-23 | IGHJ4 | IGKV3-11 | IGKJ1 | 2292 | AKEPIGQPLL | 3494 | QQRSNWP | Davide Robbiani et al., |
| | VSSYLAWYQQKPGQAPRLLIYDASNR | (Human) | (Human) | (Human) | (Human) | | WWDY | | RG | 2020 |
| | ATGIPARFSGSGSGTDFTLTISSLEPEDF | | | | | | | | | (https://www.biorxiv.or |
| | AVYYCQQRSNWPRGFGQGTKVEIK | | | | | | | | | g/content/10.1101/202 |
| | | | | | | | | | | 0.05.13.092619v2) |
| C208 | EIVLTQSPGTLSLSPGERATLSCRASQS | IGHV5-51 | IGHJ5 | IGKV3-20 | IGKJ4 | 2293 | ARGPNLQNW | 3495 | QQYGSSLT | Davide Robbiani et al., |
| | VSGSYLAWYQQRPGQAPRLLIYGASS | (Human) | (Human) | (Human) | (Human) | | FDP | | | 2020 |
| | RATGIPDRFSGSGSGTDFTLTISRLEPE | | | | | | | | | (https://www.biorxiv.or |
| | DFAVYYCQQYGSSLTFGGGTKVEIK | | | | | | | | | g/content/10.1101/202 |
| | | | | | | | | | | 0.05.13.092619v2) |
| C210 | DIQLTQSPSFLSASVGDRVTITCRASQG | IGHV3-53 | IGHJ6 | IGKV1-9 | IGKJ4 | 2294 | ARDLMAYGM | 3496 | QQLNSYP | Davide Robbiani et al., |
| | ISSYLAWYQQKPGKAPKLLIYAASTLQS | (Human) | (Human) | (Human) | (Human) | | DV | | QGT | 2020 |
| | GVPSRFSGSGSGTEFTLTISSLQPEDFA | | | | | | | | | (https://www.biorxiv.or |
| | TYYCQQLNSYPQGTFGGGTKVEIK | | | | | | | | | g/content/10.1101/202 |
| | | | | | | | | | | 0.05.13.092619v2) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| C211 | Ab | SARS-CoV2 | SARS- | SARS-CoV2 | SARS-CoV1 | S; RBD | B-cells; SARS-CoV2 | 207 | EVQLVESGGGLVQPGGSLRLSCAASEFT | 1286 |
| | | | CoV1 | | | | Human Patient | | VSSNYMSWVRQAPGKGLEWVSVIYSG | |
| | | | | | | | | | GSTFYADSVKGRFTISRDNSKNTLYLQM | |
| | | | | | | | | | NSLRPEDTAVYYCARDYGDFYFDFWGQ | |
| | | | | | | | | | GTLVTVSS | |
| C212 | Ab | SARS-CoV2 | SARS- | | SARS-CoV1, | S; non- | B-cells; SARS-CoV2 | 208 | QVQLVQSGAEVKKPGASVKVSCKASGY | 1287 |
| | | | CoV1 | | SARS-CoV2 | RBD | Human Patient | | TVTGYYIHWVRQAPGQGLEWMGWISP | |
| | | | | | | | | | NSGGTNYAQKFQGWVTMTRDMSITTA | |
| | | | | | | | | | YMELSRLRSDDTAVYYCARERYFDLGG | |
| | | | | | | | | | MDVWGQGTTVTVSS | |
| C214 | Ab | SARS-CoV2 | SARS- | | SARS-CoV1, | S; RBD | B-cells; SARS-CoV2 | 209 | QVQLVESGGGVVQPGRSLRLSCAASGF | 1288 |
| | | | CoV1 | | SARS-CoV2 | | Human Patient | | TFSSYGMHWVRQAPGKGLEWVAAIWY | |
| | | | | | | | | | DGSNKHYADSVKGRFTISRDNSKNTLYL | |
| | | | | | | | | | QMNSLRAEDTAVYYCARDVGRVTTWF | |
| | | | | | | | | | DPWGQGTLVTVSS | |
| C215 | Ab | SARS-CoV1, | | | SARS-CoV2 | S; RBD | B-cells; SARS-CoV2 | 210 | EVQLLESGGGLVQPGGSLRLSCAASGFT | 1289 |
| | | SARS-CoV2 | | | | | Human Patient | | FSSYAMSWVRQAPGKGLEWVSAITDSG | |
| | | | | | | | | | DGTFYADSVKGRFTISRDNSKNTLYLQM | |
| | | | | | | | | | NSLRAEDTAVYYCASEEDYSNYVGWFD | |
| | | | | | | | | | PWGQGTLVTVSS | |
| C216 | Ab | SARS-CoV2 | SARS- | | SARS-CoV1, | S; RBD | B-cells; SARS-CoV2 | 211 | EVQLVESGGGLVQPGGSLRLSCAASGFT | 1290 |
| | | | CoV1 | | SARS-CoV2 | | Human Patient | | FSSYDMHWVRQATGKGLEWVSAIGTA | |
| | | | | | | | | | GDTYYPDSVKGRFTISRENAKNSLYLQM | |
| | | | | | | | | | NSLRAGDTAVYYCARDRGSSGWYGWY | |
| | | | | | | | | | FDLWGRGTLVTVSS | |
| CA1 | Ab | SARS-CoV2 | | SARS-CoV2 | | S; RBD | B-cells; SARS-CoV2 | 212 | EVQLVQSGAEVKKPGASVKVSCKASGYT | 1291 |
| | | | | | | | Human Patient | | FTSYGISWVRQAPGQGLEWMGWISAY | |
| | | | | | | | | | NGNTNYAQKLQGRVTMITTDTSTSTAY | |
| | | | | | | | | | MELRSLRSDDTAVYYCAREGYCSGGSCY | |
| | | | | | | | | | SGYYYYYGMDVWGQGTTVTVSS | |
| CB6 | Ab | SARS-CoV2 | | SARS-CoV2 | | S; RBD | B-cells; SARS-CoV2 | 213 | EVQLVESGGGLVQPGGSLRLSCAASGFT | 1292 |
| | | | | | | | Human Patient | | VSSNYMSWVRQAPGKGLEWVSVIYSG | |
| | | | | | | | | | GSTFYADSVKGRFTISRDNSMNTLFLQM | |
| | | | | | | | | | NSLRAEDTAVYYCARVLPMYGDYLDYW | |
| | | | | | | | | | GQGTLVTVSS | |
| CC12.1 | Ab | SARS-CoV2 | SARS- | SARS-CoV2 | SARS-CoV1 | S; RBD | B-cells; SARS-CoV2 | 214 | EVQLVESGGGLIQPGGSLRLSCAASGLT | 1293 |
| | | | CoV1 | | | | Human Patient | | VSSNYMSWVRQAPGKGLEWVSVIYSG | |
| | | | | | | | | | GSTFYADSVKGRFTISRDNSKNTLYLQM | |
| | | | | | | | | | NSLRAEDTAVYYCARDLDVYGLDVWGQ | |
| | | | | | | | | | GTTVTVSS | |
| CC12.10 | Ab | SARS-CoV2 | SARS- | SARS-CoV2 | SARS-CoV1 | S; RBD | B-cells; SARS-CoV2 | 215 | QVQLVQSGAEVKKPGASVKVSCKASGYI | 1294 |
| | | | CoV1 | | | | Human Patient | | YSGYFMHWVRQAPGQGLEWMGWISP | |
| | | | | | | | | | DSGGANYAQTFQGRVTMTRDTSTTTAH | |
| | | | | | | | | | MELSRLRSDDTAVYYCARGPRYSGTHFD | |
| | | | | | | | | | YWGQGTLVTVSS | |
| CC12.11 | Ab | SARS-CoV2 | SARS- | SARS-CoV2 | SARS-CoV1 | S; RBD | B-cells; SARS-CoV2 | 216 | QVQLVQSGAEVKKPGASVKVSCKASGYI | 1295 |
| | | | CoV1 | | | | Human Patient | | FSGYYTHWVRQAPGQGLEWMGWISP | |
| | | | | | | | | | DSGGTNYAQKFQGRVTMTRDTSITTAY | |
| | | | | | | | | | VELSGLRSDDTAVYYCARGPRYSGTYFD | |
| | | | | | | | | | YWGQGTLVTVSS | |
| CC12.12 | Ab | SARS-CoV2 | SARS- | SARS-CoV2 | SARS-CoV1 | S; RBD | B-cells; SARS-CoV2 | 217 | QVQLVQSGAEVKKPGASVKVSCKASGYI | 1296 |
| | | | CoV1 | | | | Human Patient | | YSGYYMHWVRQAPGQGLEWMGWISP | |
| | | | | | | | | | DSGGTNYAQRFQGRVTMTRDTSTTTAY | |
| | | | | | | | | | MELSRLRSDDTAVYYCARGPRYSGTYFD | |
| | | | | | | | | | YWGQGTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| C211 | EIVMTQSPATLSVSPGERATLSCRASQ | (Human) | IGHJ4 | IGKV3-15 | IGKJ1 | 2295 | ARDYGDFYFD | 3497 | QQYNNWP | Davide Robbiani et al., |
| | SVSSNLAWYQQKPGQGPRLLIYGAST | IGHV3-66 | (Human) | (Human) | (Human) | | F | | RT | 2020 |
| | RATGIPARFSGSGSGTEFTLTISSLQSE | | | | | | | | | (https://www.biorxiv.or |
| | DFAVYYCQQYNNWPRTFGQGTKVEIK | | | | | | | | | g/content/10.1101/202 |
| | | | | | | | | | | 0.05.13.092619v2) |
| C212 | LTQPASVSGSPGQSITISCTGTSSDVGS | IGHV1-2 | IGHJ6 | IGLV2-23 | IGLJ3 | 2296 | ARERYFDLGG | 3498 | CSYAGSST | Davide Robbiani et al., |
| | YNLVSWYQQHPGKAPKLMIYEDSKRP | (Human) | (Human) | (Human) | (Human) | | MDV | | RL | 2020 |
| | SGVSNRFSGSKSGNTASLTISGLQAED | | | | | | | | | (https://www.biorxiv.or |
| | EADYYCCSYAGSSTRLFGGGTKLTVL | | | | | | | | | g/content/10.1101/202 |
| | | | | | | | | | | 0.05.13.092619v2) |
| C214 | DIQLTQSPSSLSASVGDRVTITCRASQS | IGHV3-33 | IGHJ5 | IGKV1-39 | IGKJ1 | 2297 | ARDVGRVTT | 3499 | QQSYSTPP | Davide Robbiani et al., |
| | ISSYLTWYQQKPGKAPKLLIYAASSLQS | (Human) | (Human) | (Human) | (Human) | | WFDP | | WT | 2020 |
| | GVPSRFSGSGSGTDFTLTISSLQPEDFA | | | | | | | | | (https://www.biorxiv.or |
| | TYYCQQSYSTPPWTFGQGTKVEIK | | | | | | | | | g/content/10.1101/202 |
| | | | | | | | | | | 0.05.13.092619v2) |
| C215 | DIQLTQSPSSLSASVGDRVTITCRASQS | IGHV3-23 | IGHJ5 | IGKV1-39 | IGKJ1 | 2298 | ASEEDYSNYV | 3500 | QQSYSTPP | Davide Robbiani et al., |
| | ISSYLNWYQQKPGKAPKLLIYAASSLQS | (Human) | (Human) | (Human) | (Human) | | GWFDP | | WT | 2020 |
| | GVPSRFSGSGSGTDFTLTISSLQPEDFA | | | | | | | | | (https://www.biorxiv.or |
| | TYYCQQSYSTPPWTFGQGTKVEIK | | | | | | | | | g/content/10.1101/202 |
| | | | | | | | | | | 0.05.13.092619v2) |
| C216 | DIQLTQSPSSLSASVGDRVTITCRASQS | IGHV3-13 | IGHJ2 | IGKV1-39 | IGKJ5 | 2299 | ARDRGSSGW | 3501 | QQSYSTPPI | Davide Robbiani et al., |
| | ISSYLNWYQQKPGKAPKLLIYVASSLQS | (Human) | (Human) | (Human) | (Human) | | YGWYFDL | | T | 2020 |
| | GVPSRFSGSGSGTDFTLTISSLQPEDFA | | | | | | | | | (https://www.biorxiv.or |
| | TYYCQQSYSTPPITFGQGTRLEIK | | | | | | | | | g/content/10.1101/202 |
| | | | | | | | | | | 0.05.13.092619v2) |
| CA1 | DIVMTQTPATLSLSPGERATLSCRASQ | IGHV1-18 | IGHJ6 | IGKV3-11 | IGKJ3 | 2300 | AREGYCSGGS | 3502 | QQRRNW | Rui Shi et al., 2020 |
| | SVSSYLAWYQQKPGQAPRLLIYDASN | (Human) | (Human) | (Human) | (Human) | | CYSGYYYYYG | | GT | (https://www.nature.co |
| | RATGIPARFSGSGSGTDFTLTISSLEPE | | | | | | MDV | | | m/articles/s41586-020- |
| | DFAVYYCQQRRNWGTFGPGTKVDIK | | | | | | | | | 2381- |
| | | | | | | | | | | y_reference.pdf); NCBI |
| | | | | | | | | | | (https://www.ncbi.nlm. |
| | | | | | | | | | | nih.gov/nuccore/MT470 |
| | | | | | | | | | | 194); NCBI |
| | | | | | | | | | | (https://www.ncbi.nlm. |
| | | | | | | | | | | nih.gov/nuccore/MT470 |
| | | | | | | | | | | 1945) |
| CB6 | DIVMTQSPSSLSASVGDRVTITCRASQ | IGHV3-66 | IGHJ4 | IGKV1-39 | IGKJ2 | 2301 | ARVLPMYGD | 3503 | QQSYSTPP | Rui Shi et al., 2020 |
| | SISRYLNWYQQKPGKAPKLLIYAASSL | (Human) | (Human) | (Human) | (Human) | | YLDY | | EYT | (https://www.nature.co |
| | QSGVPSRFSGSGSGTDFTLTISSLQPED | | | | | | | | | m/articles/s41586-020- |
| | FATYYCQQSYSTPPEYTFGQGTKLEIK | | | | | | | | | 2381- |
| | | | | | | | | | | y_reference.pdf); NCBI |
| | | | | | | | | | | (https://www.ncbi.nlm. |
| | | | | | | | | | | nih.gov/nuccore/MT470 |
| | | | | | | | | | | 194); NCBI |
| | | | | | | | | | | (https://www.ncbi.nlm. |
| | | | | | | | | | | nih.gov/nuccore/MT470 |
| | | | | | | | | | | 1945) |
| CC12.1 | DIVMTQSPSFLSASVGDRVTITCRASQ | IGHV3-53 | IGHJ6 | IGKV1-9 | IGKJ3 | 2302 | ARDLDVYGLD | 3504 | QQLNSYPP | Thomas Rogers et al., |
| | GISSYLAWYQQKPGKAPKLLIYAASTL | (Human) | (Human) | (Human) | (Human) | | V | | KFT | 2020 |
| | QSGVPSRFSGSGSGTEFTLTISSLQPED | | | | | | | | | (https://science.science |
| | FATYYCQQLNSYPPKFTFGPGTKVEIK | | | | | | | | | mag.org/content/early/ |
| | | | | | | | | | | 2020/06/15/science.abc |
| | | | | | | | | | | 7520); Meng Yuan et al., |
| | | | | | | | | | | 2020 |
| | | | | | | | | | | (https://www.biorxiv.or |
| | | | | | | | | | | g/content/10.1101/202 |
| | | | | | | | | | | 0.06.08.141267v1) |
| CC12.10 | QSALTQPASVSGSPGQSLTISCTGTSSD | IGHV1-2 | IGHJ4 | IGLV2-14 | IGLJ3 | 2303 | ARGPRYSGTH | 3505 | SSYAGSST | Thomas Rogers et al., |
| | VGGYNYVSWYQQHPDKAPKLMIYDV | (Human) | (Human) | (Human) | (Human) | | FDY | | QV | 2020 |
| | NNRPSGVSNRFSGSKSGSTASLTISGL | | | | | | | | | (https://science.science |
| | QAEDEADYYCSSYAGSSTQVFGGGTK | | | | | | | | | mag.org/content/early/ |
| | LTVL | | | | | | | | | 2020/06/15/science.abc |
| | | | | | | | | | | 7520) |
| CC12.11 | QSVLTQPASVSGSPGQSITISCTGTSSD | IGHV1-2 | IGHJ4 | IGLV2-14 | IGLJ3 | 2304 | ARGPRYSGTY | 3506 | SSYTSSSAQ | 2020/06/15/science.abc |
| | VGGYNYVSWYQQHPGKVPKLMIYDV | (Human) | (Human) | (Human) | (Human) | | FDY | | L | Thomas Rogers et al., |
| | SNRPSGVSNRFSGSKSGNTASLTISGL | | | | | | | | | 2020 |
| | QAEDEADYYCSSYTSSSAQLFGGGTKL | | | | | | | | | (https://science.science |
| | TVL | | | | | | | | | mag.org/content/early/ |
| | | | | | | | | | | 7520) |
| CC12.12 | QSALTQPASVSGSPGQSITISCTGTSSD | IGHV1-2 | IGHJ4 | IGLV2-14 | IGLJ3 | 2305 | ARGPRYSGTY | 3507 | SSYTSGST | Thomas Rogers et al., |
| | VGGYNYVSWYQQHPDKAPKLMIYDV | (Human) | (Human) | (Human) | (Human) | | FDY | | QV | 2020 |
| | SNRPSGVSNRFSGSKSGNTASLTISGL | | | | | | | | | (https://science.science |
| | QAEDEADYYCSSYTSGSTQVFGGGTKL | | | | | | | | | mag.org/content/early/ |
| | TVL | | | | | | | | | 2020/06/15/science.abc |
| | | | | | | | | | | 7520) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CC12.13 | Ab | SARS-CoV2 | SARS- | SARS-CoV2 | SARS-CoV1 | S; RBD | B-cells; SARS-CoV2 | 218 | EVQLVESGGGLIQPGGSLRLSCAASGFT | 1297 |
| | | | CoV1 | | | | Human Patient | | VSSNYMSWVRQAPGKGLEWVSVIYSG | |
| | | | | | | | | | GSTYYADSVKGRFTISRDNSKNTLYLQM | |
| | | | | | | | | | NSLRAEDTAVYYCARDPYGYSSIWDGQ | |
| | | | | | | | | | GGHWGQGTLVTVSS | |
| CC12.14 | Ab | SARS-CoV2 | SARS- | SARS-CoV2 | SARS-CoV1 | S; RBD | B-cells; SARS-Cov2 | 219 | QVQLVESGGGLVKPGGSLRLSCAASGFT | 1298 |
| | | | CoV1 | | | | Human Patient | | FNYYSMNWVRQAPGKGLEWISSISTSSS | |
| | | | | | | | | | FVYYADSVKGRFTISRDNAKTSLYLQMN | |
| | | | | | | | | | SLRAEDTAVYFCARGGYCSDGSCYVQDR | |
| | | | | | | | | | LIYYYSGLDVWGQGTTVTVSS | |
| CC12.15 | Ab | SARS-CoV2 | SARS- | SARS-CoV2 | SARS-CoV1 | S; RBD | B-cells; SARS-CoV2 | 220 | EVQLVESGGGLVQPGGSLRLSCAASGFT | 1299 |
| | | | CoV1 | | | | Human Patient | | FSTYEMNWVRQAPGKGLEWVSYISSSG | |
| | | | | | | | | | STIYYADSVKGRFTISRDNAKNSLYLQMS | |
| | | | | | | | | | SLRAEDTAIYYCARDRRRRYCTNGVCYR | |
| | | | | | | | | | PEEIDYWGQGTLVTVSS | |
| CC12.16 | Ab | SARS-CoV2 | SARS- | SARS-CoV2 | SARS-CoV1 | S; RBD | B-cells; SARS-CoV2 | 221 | QVQLVESGGGVVQPGRSLRLSCAASGF | 1300 |
| | | | CoV1 | (weak) | | | Human Patient | | TFSSYGMHWVRQAPGKGLEWVALIWY | |
| | | | | | | | | | DGSNKNYADSVKGRFTISRDNSKNTLDL | |
| | | | | | | | | | QMNSLRAEDTAVYYCARDPFPGAVAGT | |
| | | | | | | | | | GYLQYWGQGTLVTVSS | |
| CC12.17 | Ab | SARS-CoV2 | SARS- | SARS-CoV2 | SARS-CoV1 | S; RBD | B-cells; SARS-CoV2 | 222 | EVQLVESGGGVVQPGRSLRLSCAASGFT | 1301 |
| | | | CoV1 | (weak) | | | Human Patient | | FRNYGMHWVRQAPGKGLEGVAVISYD | |
| | | | | | | | | | GSNKYYADSVKGRFTISRDNSKNTLYLQ | |
| | | | | | | | | | MNSLRAEDTAVYYCAKSSGSYYYYYYGM | |
| | | | | | | | | | DVWGQGTTVTVSS | |
| CC12.18 | Ab | SARS-CoV1, | | SARS-CoV2 | SARS-CoV1 | S; RBD | B-cells; SARS-CoV2 | 223 | EVQLVQSGAEVKKPGASVKVSCKASGYT | 1302 |
| | | SARS-CoV2 | | (weak) | | | Human Patient | | FTSYYMHWVRQAPGQGLEWMGIINPS | |
| | | | | | | | | | GGSTSYAQKFQGRVTMTRDTSTSTVYM | |
| | | | | | | | | | ELSSLRSEDTAVYYCARLHCGGDCYLDY | |
| | | | | | | | | | WGQGTLVTVSS | |
| CC12.19 | Ab | SARS-CoV2 | SARS- | SARS-CoV2 | SARS-CoV1 | S; RBD | B-cells; SARS-CoV2 | 224 | EVQLLESGGGLVQPGGSLRLSCAASGFT | 1303 |
| | | | CoV1 | (weak) | | | Human Patient | | FSSYAMSWVRQAPGKGLEWVSAISGSG | |
| | | | | | | | | | GSTYYADSVKGRFTISRDNSKNTLYLQM | |
| | | | | | | | | | NSLRAEDTAVYYCAKGSGSGSYPNYYYY | |
| | | | | | | | | | YGMDVWGQGTTVTVSS | |
| CC12.2 | Ab | SARS-CoV2 | SARS- | SARS-CoV2 | SARS-CoV1 | S; RBD | B-cells; SARS-CoV2 | 225 | EVQLVESGGGLIQPGGSLRLSCAASGFT | 1304 |
| | | | CoV1 | | | | Human Patient | | VSSNYMSWVRQAPGKGLEWVSVFYSG | |
| | | | | | | | | | GSTYYADSVKGRFTISRDNSKNTLYLQM | |
| | | | | | | | | | NSLRAEDTAVYYCARDYGDLYFDYWGQ | |
| | | | | | | | | | GTLVTVSS | |
| CC12.20 | Ab | SARS-CoV2 | SARS- | | SARS-CoV1 | S; non- | B-cells; SARS-CoV2 | 226 | QVQLVESGGGVVQPGRSLRLSCAASGF | 1305 |
| | | | CoV1 | | | RBD | Human Patient | | TFSSYGMHWVRQAPGKGLEWVAVISY | |
| | | | | | | | | | DGSNKYYADSVKGRFTISRDNSKNTLYL | |
| | | | | | | | | | QMSSLRGEDTAVYYCAKDQAYYDILTGY | |
| | | | | | | | | | LNPPKNYYYYGMDVWGQGTTVTVSS | |
| CC12.21 | Ab | SARS-CoV2 | SARS- | | SARS-CoV1 | S; non- | B-cells; SARS-CoV2 | 227 | QVQLVQSGAEVKKPGASVKVSCKVSGY | 1306 |
| | | | CoV1 | | | RBD | Human Patient | | TLTELSMHWVRQAPGKGLEWMGGFD | |
| | | | | | | | | | PEDGETIYAQKFQGRVTMTEDTSTDTAY | |
| | | | | | | | | | MDLSSLRSEDTAVYYCATAFSIFGVVPPD | |
| | | | | | | | | | YWGQGTLVTVS | |
| CC12.23 | Ab | SARS-CoV2 | SARS- | SARS-CoV2 | SARS-CoV1 | S; non- | B-cells; SARS-CoV2 | 228 | QVQLQESGPGLVKPSETLSLTCTVSGGSI | 1307 |
| | | | CoV1 | (weak) | | RBD | Human Patient | | SSSSYYWGWIRQPPGKGLEWIGSIYYSG | |
| | | | | | | | | | STYYNPSLKSRVTISVDTSKNQFSLKLSSV | |
| | | | | | | | | | TAADTAVYYCARQGDCSTTSCAYDYWG | |
| | | | | | | | | | QGTLVTVSS | |
| CC12.24 | Ab | SARS-CoV2 | SARS- | | SARS-CoV1 | S; Unk | B-cells; SARS-CoV2 | 229 | EVQLVESGGGVVQPGRSLRLSCSASGFT | 1308 |
| | | | CoV1 | | | | Human Patient | | FSIYGMHWVRQAPGKGLEWVAVISYD | |
| | | | | | | | | | GSNKYYADSVKGRFTISRDNSKNTLYLQ | |
| | | | | | | | | | MNSLRAEDTAVYYCAKDRTGNYYYGM | |
| | | | | | | | | | DVWGQGTTVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CC12.13 | DVVMTQSPSSLSASVGDRVTITCQAS | IGHV3-53 | IGHJ4 | IGKV1-33 | IGKJ5 | 2306 | ARDPYGYSSI | 3508 | QQYDNLPI | 2020/06/15/science.abc |
| | QDISNYLNWYQQKPGKAPKLLIYDAS | (Human) | (Human) | (Human) | (Human) | | WDGQGGH | | T | Thomas Rogers et al., |
| | NFATGVPSRFSGTGSGTDFTFTISSLQP | | | | | | | | | 2020 |
| | EDIATYYCQQYDNLPITFGQGTRLEIK | | | | | | | | | (https://science.science |
| | | | | | | | | | | mag.org/content/early/ |
| | | | | | | | | | | 7520) |
| CC12.14 | DVVMTQSPLSLPVTLGQPASISCRSSQ | IGHV3-21 | IGHJ6 | IGKV2-30 | IGKJ3 | 2307 | ARGGYCSDGS | 3509 | MQGTHW | Thomas Rogers et al., |
| | SLVYSDGNTYLNWFQQRPGQSPRRLI | (Human) | (Human) | (Human) | (Human) | | CYVQDRLIYYY | | PPT | 2020 |
| | YKVSNRDSGVPDRFSGSGSGTDFTLKI | | | | | | SGLDV | | | (https://science.science |
| | SRVEAEDVGVFYCMQGTHWPPTFGQ | | | | | | | | | mag.org/content/early/ |
| | GTKVDIK | | | | | | | | | 2020/06/15/science.abc |
| | | | | | | | | | | 7520) |
| CC12.15 | QAVLTQPPSVSGAPGQRVTISCTGSSS | IGHV3-48 | IGHJ4 | IGLV1-40 | IGLJ3 | 2308 | ARDRRRRYCT | 3510 | QSYDSSLS | Thomas Rogers et al., |
| | NIEAGYDVHWYQQLPGTAPKLLIYVN | (Human) | (Human) | (Human) | (Human) | | NGVCYRPEEI | | GVV | 2020 |
| | SNRPSGVPDRFSGSKSGTSASLAITGL | | | | | | DY | | | (https://science.science |
| | QAEDEADYYCQSYDSSLSGVVFGGGT | | | | | | | | | mag.org/content/early/ |
| | KLIVL | | | | | | | | | 2020/06/15/science.abc |
| | | | | | | | | | | 7520) |
| CC12.16 | SYELTQPPSVSVAPGQTARITCGGNNI | IGHV3-33 | IGHJ4 | IGLV3-21 | IGLJ3 | 2309 | ARDPFPGAV | 3511 | QVWDSSS | Thomas Rogers et al., |
| | GSKSVHWYQQKPGQAPVLVVYDDSD | (Human) | (Human) | (Human) | (Human) | | AGTGYLQY | | DPWV | 2020 |
| | RPSGIPERFSGSNSGNTATLTISRVEAG | | | | | | | | | (https://science.science |
| | DEADYYCQVWDSSSDPWVFGGGTKL | | | | | | | | | mag.org/content/early/ |
| | TVL | | | | | | | | | 2020/06/15/science.abc |
| | | | | | | | | | | 7520) |
| CC12.17 | SYELTQPPSVSVAPGKTARITCGGNNI | IGHV3-30 | IGHJ6 | IGLV3-21 | IGLJ3 | 2310 | AKSSGSYYYYY | 3512 | QVWDSSS | Thomas Rogers et al., |
| | GSKSVHWYQQKPGQAPVLVVYDDTD | (Human) | (Human) | (Human) | (Human) | | YGMDV | | DHPVV | 2020 |
| | RPSGIPERFSGSSSGNTATLTISRVEAG | | | | | | | | | (https://science.science |
| | DEADYYCQVWDSSSDHPVVFGGGTK | | | | | | | | | mag.org/content/early/ |
| | LTVL | | | | | | | | | 2020/06/15/science.abc |
| | | | | | | | | | | 7520) |
| CC12.18 | NFMLTQPHSVSESPGKTVTISCTRSSG | IGHV1-46 | IGHJ4 | IGLV6-57 | IGLJ3 | 2311 | ARLHCGGDC | 3513 | QSYDSSNH | Thomas Rogers et al., |
| | SIANNYVKWYQQRPGSSPTTVIYEDN | (Human) | (Human) | (Human) | (Human) | | YLDY | | EEIWVV | 2020 |
| | QRPSGVPDRFSGSIDSSSNSASLTISGL | | | | | | | | | (https://science.science |
| | KTEDEADYYCQSYDSSNHEEIWVVFGG | | | | | | | | | mag.org/content/early/ |
| | GTKLIVL | | | | | | | | | 2020/06/15/science.abc |
| | | | | | | | | | | 7520) |
| CC12.19 | SYELTQSPSVSVAPGQTARITCGGNNI | IGHV3-23 | IGHJ6 | IGLV3-21 | IGLJ3 | 2312 | AKGSGSGSYP | 3514 | QVWDNNS | 2020/06/15/science.abc |
| | GSKSVHWYQRKPGQAPVLVVYDDSD | (Human) | (Human) | (Human) | (Human) | | NYYYYYGMD | | DHLV | Thomas Rogers et al., |
| | RPSGIPERFSGSNSGNTATLTISRVEAG | | | | | | V | | | 2020 |
| | DEADYYCQVWDNNSDHLVFGGGTKL | | | | | | | | | (https://science.science |
| | TVL | | | | | | | | | mag.org/content/early/ |
| | | | | | | | | | | 7520) |
| CC12.2 | EIVMTQSPGTLSLSPGERATLSCRASQ | IGHV3-53 | IGHJ4 | IGKV3-20 | IGKJ2 | 2313 | ARDYGDLYFD | 3515 | QQYGSSPR | Thomas Rogers et al., |
| | SVSSSYLAWYQQKPGQAPRLLIYGVSS | (Human) | (Human) | (Human) | (Human) | | Y | | | 2020 |
| | RATGIPDRFSGSGSETDFTLTISRLEPEE | | | | | | | | | (https://science.science |
| | FAVYYCQQYGSSPRTFGQGTKLEIK | | | | | | | | | mag.org/content/early/ |
| | | | | | | | | | | 2020/06/15/science.abc |
| | | | | | | | | | | 7520) |
| CC12.20 | QSVLTQPPSASSTPGQRVTISCSGSSSN | IGHV3-30 | IGHJ6 | IGLV1-47 | IGLJ3 | 2314 | AKDQAYYDIL | 3516 | AAWDDSL | Thomas Rogers et al., |
| | IGSNYVYWYQQLPGTAPKLLIYTNNQR | (Human) | (Human) | (Human) | (Human) | | TGYLNPPKNY | | SGRVV | 2020 |
| | PSGVPDRFSGSKSGTSASLAISGLRSED | | | | | | YYYGMDV | | | (https://science.science |
| | EADYYCAAWDDSLSGRVVFGGGTKLT | | | | | | | | | mag.org/content/early/ |
| | VL | | | | | | | | | 2020/06/15/science.abc |
| | | | | | | | | | | 7520) |
| CC12.21 | QAVVTQPPSASGTPGQRVTISCSGSSS | IGHV1-24 | IGHJ4 | IGLV1-44 | IGLJ3 | 2315 | ATAFSIFGVVP | 3517 | AAWDDSL | Thomas Rogers et al., |
| | NIGSNTVNWYQQLPGTAPKLLIYSNN | (Human) | (Human) | (Human) | (Human) | | PDY | | NGPV | 2020 |
| | QRPSGVPDRFSGSKSGTSASLAISGLQ | | | | | | | | | (https://science.science |
| | SEDEADYYCAAWDDSLNGPVFGGGT | | | | | | | | | mag.org/content/early/ |
| | KLTVL | | | | | | | | | 2020/06/15/science.abc |
| | | | | | | | | | | 7520) |
| CC12.23 | SYELTQPPSVSVSPGQTARITCSGDALP | IGHV4-39 | IGHJ4 | IGLV3-25 | IGLJ3 | 2316 | ARQGDCSTTS | 3518 | QSADSSGT | Thomas Rogers et al., |
| | KQYAYWYQQKPGQAPVLVIYKDSERP | (Human) | (Human) | (Human) | (Human) | | CAYDY | | YLVV | 202 |
| | SGIPERFSGSSSGTTATLTISGVQAEDE | | | | | | | | | (https://science.science |
| | ADYYCQSADSSGTYLVVFGGGTKLAV | | | | | | | | | mag.org/content/early/ |
| | | | | | | | | | | 2020/06/15/science.abc |
| | | | | | | | | | | 7520) |
| CC12.24 | DIQLTQSPSSLSASVGDRVTITCRASQS | IGHV3-30 | IGHJ6 | IGKV1-39 | IGKJ1 | 2317 | AKDRTGNYYY | 3519 | QQSYSTP | Thomas Rogers et al., |
| | ISSYLNWYQQKPGKAPKLLIYAASNLQ | (Human) | (Human) | (Human) | (Human) | | GMDV | | WT | 2020 |
| | SGVPSRFSGSGSGTDFTLTISSLQPEDF | | | | | | | | | (https://science.science |
| | ATYYCQQSYSTPWTFGQGTKVEIK | | | | | | | | | mag.org/content/early/ |
| | | | | | | | | | | 2020/06/15/science.abc |
| | | | | | | | | | | 7520) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CC12.25 | Ab | SARS-CoV1, | | | SARS-CoV1 | S; non- | B-cells; SARS-CoV2 | 230 | QVQLVESGGGLVQPGGSLRLSCAASGF | 1309 |
| | | SARS-CoV2 | | | | RBD | Human Patient | | TFSSYAMSWVRQAPGKGLEWVSAISGS | |
| | | | | | | | | | GDSTYYADSVKGRFTISRDNSKNTLYLQ | |
| | | | | | | | | | MNSLRAEDTAVYYCAKDRYYEFWSGYS | |
| | | | | | | | | | NWFDPWGQGTLVTISS | |
| CC12.26 | Ab | SARS-CoV2 | SARS- | | SARS-CoV1 | S; non- | B-cells; SARS-CoV2 | 231 | EVQLVQSGAEVKKPGESLKISCKGSGYSF | 1310 |
| | | | CoV1 | | | RBC | Human Patient | | TSYWIGWVRQMPGKGLEWMGIIYPGD | |
| | | | | | | | | | SDTRYSPSFQGQVTVSADKSISTAYLQW | |
| | | | | | | | | | SSLKASDTAMYYCARVNYYDSSGYPSFH | |
| | | | | | | | | | FDYWGQGTLVTVS | |
| CC12.27 | Ab | SARS-CoV1, | | | SARS-CoV1 | S; non- | B-cells; SARS-CoV2 | 232 | QVQLVQSGAEVKKPGASVKVSCKASGY | 1311 |
| | | SARS-CoV2 | | | | RBD | Human Patient | | TFIGYYMHWVRQAPGQGLEWMGWIN | |
| | | | | | | | | | PNSGGTNYAQKFQGRVTMTRDTSISTV | |
| | | | | | | | | | YMELSRLRSDDTAVYYCAREMPAAMGY | |
| | | | | | | | | | YYYGMDVWGQGTTVTVSS | |
| CC12.28 | Ab | SARS-CoV1, | | SARS-CoV2 | | S; RBD | B-cells; SARS-CoV2 | 233 | EVQLLESGGGLVQPGGSLRLSCAASGFT | 1312 |
| | | SARS-CoV2 | | and SARS- | | | Human Patient | | FSNYAMTWVRQAPGKGLEWVSAISSGS | |
| | | | | CoV1 | | | | | GSTYYADSVKGRFTISRDNSKNTVYLQM | |
| | | | | | | | | | NSLRAEDTAIYYCAKANKYSSSEFDFWG | |
| | | | | | | | | | QGTLVTISS | |
| CC12.3 | Ab | SARS-CoV2 | SARS- | SARS-CoV2 | SARS-CoV1 | S; RBD | B-cells; SARS-CoV2 | 234 | QVQLVESGGGLIQPGGSLRLSCAASGFT | 1313 |
| | | | CoV1 | | | | Human Patient | | VSSNYMSWVRQAPGKGLEWVSVIYSG | |
| | | | | | | | | | GSTFYADSVKGRFTISRDNSKSTLYLQM | |
| | | | | | | | | | NSLRVEDTAVYYCARDFGDFYFDYWGQ | |
| | | | | | | | | | GTLVTVSS | |
| CC12.4 | Ab | SARS-CoV2 | SARS- | SARS-CoV2 | SARS-CoV1 | S; RBD | B-cells; SARS-CoV2 | 235 | QVQLVQSGAEVKKPGASVKVSCKASGY | 1314 |
| | | | CoV1 | | | | Human Patient | | TFTGYYMHWVRQAPGQGLEWMGWIS | |
| | | | | | | | | | PNSGGTNYAQKFQGWVTMTRDTSVST | |
| | | | | | | | | | AYMELSRLRFDDTAVYYCATESWVYGS | |
| | | | | | | | | | GSYSSGAFDIWGQGTMVTVSS | |
| CC12.5 | Ab | SARS-CoV2 | SARS- | SARS-CoV2 | SARS-CoV1 | S; RBD | B-cells; SARS-CoV2 | 236 | EVQLVQSGAEVKKPGASVKVSCKASGYI | 1315 |
| | | | CoV1 | | | | Human Patient | | YSGYYMHWVRQAPGQGLEWMGWISP | |
| | | | | | | | | | DSGGTNYAQRFQGRVTMTRDTSTTTAY | |
| | | | | | | | | | MELSRLRSDDTAMYYCARGPRYSGTYF | |
| | | | | | | | | | DYWGQGTLVTVSS | |
| CC12.6 | Ab | SARS-CoV2 | SARS- | SARS-CoV2 | SARS-CoV1 | S; RBD | B-cells; SARS-CoV2 | 237 | QVQLVQSGAEVKKPGASVKVSCKASGYI | 1316 |
| | | | CoV1 | | | | Human Patient | | FSGYYMHWVRQAPGQGLEWMGWISP | |
| | | | | | | | | | DSGGTNYAQKFQGRVTMTRDTSITTGY | |
| | | | | | | | | | MELSGLRSDDTAVYYCARGPRYSGTYFD | |
| | | | | | | | | | YWGQGTLVTVSS | |
| CC12.7 | Ab | SARS-CoV2 | SARS- | SARS-CoV2 | SARS-CoV1 | S; RBD | B-cells; SARS-CoV2 | 238 | EVQLVQSGAEVKKPGASVKVTCKTSGYI | 1317 |
| | | | CoV1 | | | | Human Patient | | FSGYYMHWVRQVPGQGLEWMGWISP | |
| | | | | | | | | | DSGATNYAQKFQGRVTMTRDTSITTSYV | |
| | | | | | | | | | ELTWLKSDDTAVYYCARGPRYSGTYFDF | |
| | | | | | | | | | WGQGTLVTVSS | |
| CC12.8 | Ab | SARS-CoV2 | SARS- | SARS-CoV2 | SARS-CoV1 | S; RBD | B-cells; SARS-CoV2 | 239 | QVQLVQSGAEVKKPGASVKVSCKASGYI | 1318 |
| | | | CoV1 | | | | Human Patient | | FSGYYTHWVRQAPGQGLEWMGWISP | |
| | | | | | | | | | DSGGTNYAQKFQGRVTMTRDTSITTAY | |
| | | | | | | | | | VELSGLRSDDTAVYYCARGPRYSGTYFD | |
| | | | | | | | | | YWGQGTLVTVSS | |
| CC12.9 | Ab | SARS-CoV2 | SARS- | SARS-CoV2 | SARS-CoV1 | S; non- | B-cells; SARS-CoV2 | 240 | EVQLVQSGAEVKKPGASVKVSCKASGYI | 1319 |
| | | (weak) | CoV1 | | | RBD | Human Patient | | FSGYYMHWVRQAPGQGLEWMGWISP | |
| | | | | | | | | | DSGGTNYAQNFQGRVTMTRDTSISTGY | |
| | | | | | | | | | MELSRLRSDDTAMYYCARGPRYSGTYF | |
| | | | | | | | | | DYWGQGVLVTVSS | |
| CC6.29 | Ab | SARS-CoV2 | | SARS-CoV2 | | S; RBD | B-cells; SARS-CoV2 | 241 | QVQLVQSGSELKKPGASVKVSCKASGYT | 1320 |
| | | | | | | | Human Patient | | FATYALNWVRQAPGQGLEWMGWVNT | |
| | | | | | | | | | NTGSPTYAQGFTGRFVFSFDTSVSTAYL | |
| | | | | | | | | | QIRTLKAEDTAVYYCAVYYYDSGSPGWF | |
| | | | | | | | | | DPWGQGTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CC12.25 | QSALTQPPSASGTPGQRVTISCSGSSS | IGHV3-23 | IGHJ5 | IGLV1-44 | IGLJ3 | 2318 | AKDRYYEFWS | 3520 | AAWDDSL | Thomas Rogers et al., |
| | NIGSNTVNWYQQLPGTAPKVLVYSND | (Human) | (Human) | (Human) | (Human) | | GYSNWFDP | | NGPV | 2020 |
| | QRPSGVPDRFSGSKSGTSASLAISGLQ | | | | | | | | | (https://science.science |
| | SEDEADYYCAAWDDSLNGPVFGGGT | | | | | | | | | mag.org/content/early/ |
| | KLTVL | | | | | | | | | 2020/06/15/science.abc |
| | | | | | | | | | | 7520) |
| CC12.26 | QTVVTQEPSLTVSPGGTVTLTCVSSTG | IGHV5-51 | IGHJ4 | IGLV7-43 | IGLJ3 | 2319 | ARVNYYDSSG | 3521 | LLYYGGAQ | Thomas Rogers et al., |
| | AVTSGYYPNWFQQKPGQAPRALIYST | (Human) | (Human) | (Human) | (Human) | | YPSFHFDY | | RWV | 2020 |
| | SKKHSWTPARFSGSLLGGKAALTLSGV | | | | | | | | | (https://science.science |
| | QPEDEAEYYCLLYYGGAQRWVFGGG | | | | | | | | | mag.org/content/early/ |
| | TKLTVL | | | | | | | | | 2020/06/15/science.abc |
| | | | | | | | | | | 7520) |
| CC12.27 | QSVLTQPASVSGSPGQSITISCTGTSSD | IGHV1-2 | IGHJ6 | IGLV3-23 | IGLJ1 | 2320 | AREMPAAM | 3522 | YSYAGSSTF | Thomas Rogers et al., |
| | VGSYNLVSWYQQHPGKAPKLMIYEGS | (Human) | (Human) | (Human) | (Human) | | GYYYYGMDV | | V | 2020 |
| | KRPSGVSNRFSGSKSGNTASLTISGLQ | | | | | | | | | (https://science.science |
| | AEDEADYYCYSYAGSSTFVFVFGTGTK | | | | | | | | | mag.org/content/early/ |
| | VIVL | | | | | | | | | 2020/06/15/science.abc |
| | | | | | | | | | | 7520) |
| CC12.28 | QSALTQPPSASGTPGQRVTISCSGSSS | IGHV3-23 | IGHJ4 | IGLV1-47 | IGLJ3 | 2321 | AKANKYSSSE | 3523 | AAWDDSL | Thomas Rogers et al., |
| | NIGSNYVYWYQQLPGAAPKLLIYRND | (Human) | (Human) | (Human) | (Human) | | FDF | | SGWV | 2020 |
| | QRPSGVPDRFSGSKSGTSVSLAISGLRS | | | | | | | | | (https://science.science |
| | EDEADYYCAAWDDSLSGWVFGGGTK | | | | | | | | | mag.org/content/early/ |
| | LTVL | | | | | | | | | 2020/06/15/science.abc |
| | | | | | | | | | | 7520) |
| CC12.3 | EIVLTQSPGTLSLSPGERATLSCRASQS | IGHV3-53 | IGHJ4 | IGKV3-20 | IGKJ2 | 2322 | ARDFGDFYFD | 3524 | QQYGSSPR | Thomas Rogers et al., |
| | VSSYLAWYQQKPGQAPRLLIYGASSRA | (Human) | (Human) | (Human) | (Human) | | Y | | T | 2020 |
| | TGIPDRFSGSGSGTDFTLTISRLEPEDF | | | | | | | | | (https://science.science |
| | AVYYCQQYGSSPRTFGQGTKLEIK | | | | | | | | | mag.org/content/early/ |
| | | | | | | | | | | 2020/06/15/science.abc |
| | | | | | | | | | | 7520); Meng Yuan et al., |
| | | | | | | | | | | 2020 |
| | | | | | | | | | | (https://www.biorxiv.or |
| | | | | | | | | | | g/content/10.1101/202 |
| | | | | | | | | | | 0.06.08.141267v1) |
| CC12.4 | QSVLTQPPSASGSPGQSVTISCTGTSS | IGHV1-2 | IGHJ3 | IGLV2-8 | IGLJ3 | 2323 | ATESWVYGS | 3525 | TSYAGSNN | Thomas Rogers et al., |
| | DVGGYDYVSWYQQHPGKAPKLMIYE | (Human) | (Human) | (Human) | (Human) | | GSYSSGAFDI | | FV | 2020 |
| | VSKRPSGVPDRFSGSKSGNTASLTVPG | | | | | | | | | (https://science.science |
| | LQAEDEADYYCTSYAGSNNFVFGGGT | | | | | | | | | mag.org/content/early/ |
| | KLIVL | | | | | | | | | 2020/06/15/science.abc |
| | | | | | | | | | | 7520) |
| CC12.5 | QTVVTQPASVSGSPGQSITISCTGTSSD | IGHV1-2 | IGHJ4 | IGLV2-14 | IGLJ3 | 2324 | ARGPRYSGTY | 3526 | SSYTSSSTQ | Thomas Rogers et al., |
| | VGGYNYVSWYQQHPDKAPKLIIYDVS | (Human) | (Human) | (Human) | (Human) | | FDY | | V | 2020 |
| | NRPSGVSNRFSGSKSGNTASLTISGLQ | | | | | | | | | (https://science.science |
| | AEDEAAYYCSSYTSSSTQVFGGGTKLT | | | | | | | | | mag.org/content/early/ |
| | VL | | | | | | | | | 2020/06/15/science.abc |
| | | | | | | | | | | 7520) |
| CC12.6 | QSALTQPTSVSGSPGQSITISCTGTSSD | IGHV1-2 | IGHJ4 | IGLV2-14 | IGLJ3 | 2325 | ARGPRYSGTY | 3527 | SSYTSSSTQ | Thomas Rogers et al., |
| | VGGYNYVSWYQQHPGKAPKLIIYDVS | (Human) | (Human) | (Human) | (Human) | | FDY | | V | 2020 |
| | NRPSGVSNRFSGSKSGNTASLTISGLQ | | | | | | | | | (https://science.science |
| | AEDEADYYCSSYTVSSTQVFGGGTKLT | | | | | | | | | mag.org/content/early/ |
| | VL | | | | | | | | | 2020/06/15/science.abc |
| | | | | | | | | | | 7520) |
| CC12.7 | QPVLTQPASVSGSPGQSLTISCTGTSS | IGHV1-2 | IGHJ4 | IGLV2-14 | IGLJ3 | 2326 | ARGPRYSGTY | 3528 | SSYTVSSTQ | Thomas Rogers et al. |
| | DIGGFNYVSWYQQHPGKAPKLMIFD | (Human) | (Human) | (Human) | (Human) | | FDY | | V | 2020 |
| | VSKRPSGVPNRFSGSKSGNTASLTISGL | | | | | | | | | (https://science.science |
| | QAEDEGDYYCSSYTISSAQVFGGGTKL | | | | | | | | | mag.org/content/early/ |
| | TVL | | | | | | | | | 2020/06/15/science.abc |
| | | | | | | | | | | 7520) |
| CC12.8 | QSALTQPASVSGSPGQSITISCTGTSSD | IGHV1-2 | IGHJ4 | IGLV2-14 | IGLJ3 | 2327 | ARGPRYSGTY | 3529 | SSYTSSSAQ | Thomas Rogers et al., |
| | VGGYNHVSWYQQHPGKAPKLMIYDV | (Human) | (Human) | (Human) | (Human) | | FDY | | L | 2020 |
| | SNRPSGVSNRFSGSKSGNTASLTISGL | | | | | | | | | (https://science.science |
| | QAEDEADYYCSSYTSSSAQLFGGGTKL | | | | | | | | | mag.org/content/early/ |
| | TVL | | | | | | | | | 2020/06/15/science.abc |
| | | | | | | | | | | 7520) |
| CC12.9 | QSVLTQPASVSGSPGQSITISCTGTSSD | IGHV1-2 | IGHJ4 | IGLV2-14 | IGLJ3 | 2328 | ARGPRYSGTY | 3530 | SSYTSSSAQ | Thomas Rogers et al., |
| | VGGYNYVSWYQQHPGKVPKLMIYDV | (Human) | (Human) | (Human) | (Human) | | FDY | | V | 2020 |
| | SNRPSGVSNRFSGSKSGNTASLTISGL | | | | | | | | | (https://science.science |
| | QAEDEADYYCSSYTSSSAQVFGGGTKL | | | | | | | | | mag.org/content/early/ |
| | TVL | | | | | | | | | 2020/06/15/science.abc |
| | | | | | | | | | | 7520) |
| CC6.29 | DIQLTQSPSSLSASVGDRVTITCRASQT | IGHV7-4- | IGHJ5 | IGKV1-39 | IGKJ3 | 2329 | AVYYYDSGSP | 3531 | QQSYSTPP | Thomas Rogers et al., |
| | ASSYLNWYQQKPGKAPNLLIYAASSLQ | 1 | (Human) | (Human) | (Human) | | GWFDP | | T | 2020 |
| | SGVPSRFSGSGSVTDFTLTISSLQPEDF | (Human) | | | | | | | | (https://science.science |
| | ATYYCQQSYSTPPTFGQGTKVDIK | | | | | | | | | mag.org/content/early/ |
| | | | | | | | | | | 2020/06/15/science.abc |
| | | | | | | | | | | 7520) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CC6.30 | Ab | SARS-CoV2 | | SARS-CoV2 | | S; RBD | B-cells; SARS-CoV2 | 242 | QVQLVQSGAEVKKPGSSVKVSCKASGG | 1321 |
| | | | | | | | Human Patient | | TFSIYAITWVRQAPGQGLEWMGGIIPII | |
| | | | | | | | | | GTANYAQKFQGRVTITADKSTSTAYMEL | |
| | | | | | | | | | SSLRSEDTAVYYCARDFRYCSSTRCYFWF | |
| | | | | | | | | | DPWGQGTLVTVSS | |
| CC6.31 | Ab | SARS-CoV2 | | SARS-CoV2 | | S; RBD | B-cells; SARS-CoV2 | 243 | EVQLVQSGAEVKKPGASVKVSCMASGY | 1322 |
| | | | | | | | Human Patient | | TFTSYYMHWVRQAPGQGLEWMGIISP | |
| | | | | | | | | | SGGGTSYAQKFQGRVTLTRDTSTSTVYM | |
| | | | | | | | | | ELSSLRSEDTAVYYCARWYDSTGSIDYW | |
| | | | | | | | | | GQGTLVTVSS | |
| CC6.32 | Ab | SARS-CoV1, | | SARS-CoV2 | | S; RBD | B-cells; SARS-CoV2 | 244 | EVQLVESGGGLVQPGRSLRLSCAASGFT | 1323 |
| | | SARS-CoV2 | | | | | Human Patient | | FDDYAMHWVRQAPGKGLEWVSGISW | |
| | | | | | | | | | NSGSIAFAGSVKGRFTISRDNAKNSLYLQ | |
| | | | | | | | | | MNSLRAEDTALYYCAKDQGYSYGNYFD | |
| | | | | | | | | | YWGQGTLVTVSS | |
| CC6.33 | Ab | SARS-CoV1, | | SARS-CoV2 | | S; RBD | B-cells; SARS-CoV2 | 245 | QVQLVQSGAEVKKPGSSVKVSCKASGG | 1324 |
| | | SARS-CoV2 | | and SARS- | | | Human Patient | | TFSSSAISWVRQAPGQGLEWMGGIIPIL | |
| | | | | CoV1 | | | | | DITNYAQKFQGRVTITADKSTSTAFMELS | |
| | | | | | | | | | SLRSEDTAVYYCALRNQWDLLVYWGQG | |
| | | | | | | | | | TLVTVSS | |
| Clone11- | Ab | SARS-CoV2 | | | | S; RBD | Immunised Mouse | 246 | QVQLQQPGAELVMPGASVKMSCKASG | 1325 |
| 9 | | | | | | | | | YTFTDYWMHWVKQRPGQGLEWIGAE | |
| | | | | | | | | | DTSDSYTSYNQKFKGKATLTVDESSSTAY | |
| | | | | | | | | | MQLSSLTSEDSAVYYCARRGYGSSYTWF | |
| | | | | | | | | | AYWGQGTLVTVSA | |
| CnC2t1p1_ | Ab | SARS-CoV2 | | | SARS-CoV2 | S; Unk | B-cells; SARS-CoV2 | 247 | QVQLVQSGAEVKKPGSSVKVSCKASGG | 1326 |
| B10 | | | | | | | Human Patient | | TFSSYAISWVRQAPGQGLEWMGGIIPIF | |
| | | | | | | | | | GTANYAQKFQGRVTITADKSTSTAYMEL | |
| | | | | | | | | | SSLRSEDTAVYYCARVSGYDSSGYWGDY | |
| | | | | | | | | | WGQGTLVTVSS | |
| CnC2t1p1_ | Ab | SARS-CoV2 | | SARS-CoV2 | | S; RBD | B-cells; SARS-CoV2 | 248 | QVQLVQSGAEVKKPGASVKVSCKASGY | 1327 |
| B4 | | | | | | | Human Patient | | TFTSYGISWVRQAPGQGLEWMGWISA | |
| | | | | | | | | | YNGNTNYAQKLQGRVTMTTDTSTSTAY | |
| | | | | | | | | | MELRSLRSDDTAVYYCARDGELLGWFD | |
| | | | | | | | | | PWGQGTLVTVSS | |
| CnC2t1p1_ | Ab | SARS-CoV2 | | SARS-CoV2 | | S; RBD | B-cells; SARS-CoV2 | 249 | EVQLVESGGGLVQPGRSLRLSCTASGFT | 1328 |
| D6 | | | | | | | Human Patient | | FGDYAMSWFRQAPGKGLEWVGFIRSK | |
| | | | | | | | | | AYGGTTEYAASVKGRFTISRDDSKSIAYL | |
| | | | | | | | | | QMNSLKTEDTAVYYCTRVRRLWFGSYY | |
| | | | | | | | | | YGMDVWGQGTTVTVSS | |
| CnC2t1p1_ | Ab | SARS-CoV2 | | SARS-CoV2 | | S; RBD | B-cells; SARS-CoV2 | 250 | EVQLVESGGGLVQPGRSLRLSCTASGFT | 1329 |
| E12 | | | | | | | Human Patient | | FGDYAMSWFRQAPGKGLEWVGFIRSK | |
| | | | | | | | | | AYGGTTEYAASVKGRFTISRDDSKSIVYL | |
| | | | | | | | | | QMNSLKTEDTAVYYCTRVRRLWFGSYY | |
| | | | | | | | | | YGMDVWGQGTTVTVSS | |
| CnC2t1p1_ | Ab | SARS-CoV2 | | SARS-CoV2 | | S; RBD | B-cells; SARS-CoV2 | 251 | QVQLVQSGAEVKNPGASVKVSCKASGY | 1330 |
| E8 | | | | | | | Human Patient | | IFTNYYIHWVRQAPGQGLEWVGWIHSL | |
| | | | | | | | | | SGGTSYAQKFQGRVTLTRDASIRTAYME | |
| | | | | | | | | | LSRLGSDDTALYYCARASVSTITDFDYWG | |
| | | | | | | | | | QGTLVAVSS | |
| CnC2t1p1_ | Ab | SARS-CoV2 | | SARS-CoV2 | | S; RBD | B-cells; SARS-CoV2 | 252 | QVQLVQSGAEVKKPGASVKVSCKASGYI | 1331 |
| G6 | | | | | | | Human Patient | | FTNYYIHWVRQAPGQGLEWVGWIHSL | |
| | | | | | | | | | SGGTSYAQKFQGRVTLTRDAPIRTAYME | |
| | | | | | | | | | LSGLGSDDTAVYYCARASVATITDFDYW | |
| | | | | | | | | | GQGTLVAVSS | |
| CoV2- | Ab | SARS-CoV1, | | | SARS-CoV2 | S; non- | B-cells; SARS-CoV2 | 253 | QVQLVESGGGVVQPGRSLRLSCAASGF | 1332 |
| 2006 | | SARS-CoV2 | | | | RBC | Human Patient | | TFSYYAILWFRQAPGKGLEWVAIISYDGS | |
| | | | | | | | | | NKYYADSVKGRFTISRDNSKNTLYLQMN | |
| | | | | | | | | | SLRPEDTAVYYCARPQSGGYYAPLDYW | |
| | | | | | | | | | GQGTLVTVSS | |
| CoV2- | Ab | SARS-CoV1, | | | SARS-CoV2 | S; RBD | B-cells; SARS-CoV2 | 254 | QVQLVESGGGVVQPGRSLRLSCAASGF | 1333 |
| 2007 | | SARS-CoV2 | | | | | Human Patient | | TFSRYGMHWVRQAPGKGLEWVGIISYD | |
| | | | | | | | | | ASDKTYAESVKGRFTISRDNSKNTLYLQ | |
| | | | | | | | | | MNSLRAEDTAVYYCAKVSATYYYYYYG | |
| | | | | | | | | | MDVWGQGTTVTVSS | |
| CoV2- | Ab | SARS-CoV1, | | | SARS-CoV2 | S; non- | B-cells; SARS-CoV2 | 255 | QVQLVESGGGVVQPGRSLRLSCAASGF | 1334 |
| 2009 | | SARS-CoV2 | | | | RBD | Human Patient | | TFSTYAMHWVRQAPGKGLEWVAVISY | |
| | | | | | | | | | DGSNKYYADSVKGRFTISRDNPKNTLYL | |
| | | | | | | | | | QMNSLRAEDTAVYYCARDTATYVLLWS | |
| | | | | | | | | | GDFNLDYWGQGTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CC6.30 | DIQMTQSPSSLSASVGDRVTITCRASQ | IGHV1-69 | IGHJ5 | IGKV1-39 | IGKJ3 | 2330 | ARDFRYCSST | 3532 | QQSYSTPR | Thomas Rogers et al., |
| | NISSYLNWYQQEAGKAPKLLIYAASSL | (Human) | (Human) | (Human) | (Human) | | RCYFWFDP | | T | 2020 |
| | QSGVPSRFSGSGSGTDFTLTISSLQPED | | | | | | | | | (https://science.science |
| | FATYYCQQSYSTPRTFGQGTKVDIK | | | | | | | | | mag.org/content/early/ |
| | | | | | | | | | | 2020/06/15/science.abc |
| | | | | | | | | | | 7520) |
| CC6.31 | DIQMTQSPSSLSASVGDRVTITCRASQ | IGHV1-46 | IGHJ4 | IGKV1-17 | IGKJ4 | 2331 | ARWYDSTGSI | 3533 | LQHNSYPIL | Thomas Rogers et al., |
| | GIRNDLGWYQQKPGKAPKRLIYAASSL | (Human) | (Human) | (Human) | (Human) | | DY | | T | 2020 |
| | QSGVPSRFSGSGSGTEFTLTISSLQPED | | | | | | | | | (https://science.science |
| | FATYYCLQHNSYPILTFGGGTKLEIK | | | | | | | | | mag.org/content/early/ |
| | | | | | | | | | | 2020/06/15/science.abc |
| | | | | | | | | | | 7520) |
| CC6.32 | QLVLTQPPSVSVAPGKTARITCGGNNI | IGHV3-9 | IGHJ4 | IGLV3-21 | IGLJ6 | 2332 | AKDQGYSYG | 3534 | QVWDSSS | Thomas Rogers et al., |
| | GSKSVYWYQQKPGQAPVLVVYDDSD | (Human) | (Human) | (Human) | (Human) | | NYFDY | | DHPYV | 2020 |
| | RPSGIPERFSGSNSGNTATLTISRVEAG | | | | | | | | | (https://science.science |
| | DEADYYCQVWDSSSDHPYVFGSGTKV | | | | | | | | | mag.org/content/early/ |
| | TVL | | | | | | | | | 2020/06/15/science.abc |
| | | | | | | | | | | 7520) |
| CC6.33 | EIVLTQSPGTLSLSPGERATLSCRASQS | IGHV1-69 | IGHJ4 | IGKV3-20 | IGKJ1 | 2333 | ALRNQWDLL | 3535 | QHYGSSL | Thomas Rogers et al., |
| | VSSSYLAWYQQKPGQAPRLLIYGASSR | (Human) | (Human) | (Human) | (Human) | | VY | | WT | 2020 |
| | ATGIPDRFSGSGSGTDFTLTISRLEPED | | | | | | | | | (https://science.science |
| | FAVYYCQHYGSSLWTFGQGTKLEIK | | | | | | | | | mag.org/content/early/ |
| | | | | | | | | | | 2020/06/15/science.abc |
| | | | | | | | | | | 7520) |
| Clone11- | DVLMTQTPLSLPVSLGDQASISCRSSQ | IGHV1-69 | IGHJ3 | IGKV1-117 | IGKJ5 | 2334 | ARRGYGSSYT | 3536 | FQGSHVPL | Nadezhda Antipova et |
| 9 | SIVHSNGNTYLEWYLQKPGQSPKLLIY | (Mouse) | (Mouse) | (Mouse) | (Mouse) | | WFAY | | T | al., 2020 |
| | KVSNRFSGVPDRFSGSGSGTDFTLKIS | | | | | | | | | |
| | RVEAEDLGVYYCFQGSHVPLTFGAGT | | | | | | | | | |
| | KLELK | | | | | | | | | |
| CnC2t1p1_ | EIVLTQSPATLSLSPGERATLSCRASQS | IGHV1-69 | IGHJ4 | IGKV3-11 | IGKJ4 | 2335 | ARVSGYDSSG | 3537 | QQRSNWP | Christoph Kreer et al., |
| B10 | VSSYLAWYQQKPGQAPRLLIYDASNR | (Human) | (Human) | (Human) | (Human) | | YWGDY | | PALT | 2020 |
| | ATGIPARFSGSGSGTDFTLTISSLEPEDF | | | | | | | | | (https://doi.org/10.101 |
| | AVYYCQQRSNWPPALTFGGGTKVEIK | | | | | | | | | 6/j.cell.2020.06.044) |
| CnC2t1p1_ | QSALTQPASVSGSPGQSITISCTGTSSD | IGHV1-18 | IGHJ5 | IGLV2-23 | IGLJ3 | 2336 | ARDGELLGW | 3538 | CSYAGSST | Christoph Kreer et al., |
| B4 | VGSYNLVSWYQQHPGKAPKLMIYEGS | (Human) | (Human) | (Human) | (Human) | | FDP | | WV | 2020 |
| | KRPSGVSNRFSGSKSGNTASLTISGLQ | | | | | | | | | (https://doi.org/10.101 |
| | AEDEADYYCCSYAGSSTWVFGGGTKL | | | | | | | | | 6/j.cell.2020.06.044) |
| | TVL | | | | | | | | | |
| CnC2t1p1_ | DIVMTQSPLSLPVTPGEPASISCRSSQS | IGHV3-49 | IGHJ6 | IGKV2-28 | IGKJ3 | 2337 | TRVRRLWFGS | 3539 | MQALQTP | Christoph Kreer et al., |
| D6 | LLHSNGYNYLDWYLQKPGQSPQLLIYL | (Human) | (Human) | (Human) | (Human) | | YYYGMDV | | GT | 2020 |
| | GSNRASGVPDRFSGSGSGTDFTLKISR | | | | | | | | | (https://doi.org/10.101 |
| | VEAEDVGVYYCMQALQTPGTFGPGT | | | | | | | | | 6/j.cell.2020.06.044) |
| | KVDIK | | | | | | | | | |
| CnC2t1p1_ | DIVMTQSPLSLPVTPGEPASISCRSSQS | IGHV3-49 | IGHJ6 | IGKV2-28 | IGKJ3 | 2338 | TRVRRLWFGS | 3540 | MQALQTP | Christoph Kreer et al., |
| E12 | LLHSNGYNYLDWYLQKPGQSPQLLIYL | (Human) | (Human) | (Human) | (Human) | | YYYGMDV | | GT | 2020 |
| | GSNRASGVPDRFSGSGSGTDFTLKISR | | | | | | | | | (https://doi.org/10.101 |
| | VEAEDVGVYYCMQALQTPGTFGPGT | | | | | | | | | 6/j.cell.2020.06.044) |
| | KVDIK | | | | | | | | | |
| CnC2t1p1_ | QSALTQPASVSGSPGQSITISCTGTSGD | IGHV1-2 | IGHJ4 | IGLV2-23 | IGLJ3 | 2339 | ARASVSTITDF | 3541 | CSYAGVRT | Christoph Kreer et al., |
| E8 | VGSYNLVSWYQQHPGKAPKLVIYEAT | (Human) | (Human) | (Human) | (Human) | | DY | | VV | 2020 |
| | KRPSGVSNRFFASKSGNTASLTISGLQ | | | | | | | | | (https://doi.org/10.101 |
| | AEDEADYYCCSYAGVRTVVFGGGTKL | | | | | | | | | 6/j.cell.2020.06.044) |
| | TVL | | | | | | | | | |
| CnC2t1p1_ | QSALTQPASVSGSPGQSITISCTGTSGD | (Human) | IGHJ4 | IGLV2-23 | IGLJ3 | 2340 | ARASVATITDF | 3542 | CSYAGVRT | Christoph Kreer et al., |
| G6 | IGSYNLVSWYQQYPGKAPKLIIYEASKR | IGHV1-2 | (Human) | (Human) | (Human) | | DY | | VV | 2020 |
| | PSGVSNRFFASKSGNTASLTISGLQAE | | | | | | | | | (https://doi.org/10.101 |
| | DEADYYCCSYAGVRTVVFGGGTKLTVL | | | | | | | | | 6/j.cell.2020.06.044) |
| CoV2- | EIVLTQSPGTLSLSPGERATLSCRASQS | IGHV3-30- | IGHJ4 | IGKV3-20 | IGKJ1 | 2341 | ARPQSGGYYA | 3543 | QQYGSSP | Seth Zost et al., 2020 |
| 2006 | VSSSYLAWYQQKPGQAPRLLIYGASSR | 3 (Human) | (Human) | (Human) | (Human) | | PLDY | | WT | (https://www.nature.co |
| | ATGIPDRFSGSGSGTDFTLTISRLEPED | | | | | | | | | m/articles/s41591-020- |
| | FAVYYCQQYGSSPWTFGQGTKVEIK | | | | | | | | | 0998-x) |
| CoV2- | DIQMTQSPSSLSASVGDRVTITCQASQ | IGHV3-30 | IGHJ6 | IGKV1-33 | IGKJ3 | 2342 | AKVSATYYYYY | 3544 | QQYDNLLF | Seth Zost et al., 2020 |
| 2007 | DISTYLNWYQQKPGKAPKLLIYDASNL | (Human) | (Human) | (Human) | (Human) | | YGMDV | | T | (https://www.nature.co |
| | ETGVPSRFSGSGSGTDFTFTISSLQPED | | | | | | | | | m/articles/s41591-020- |
| | IATYYCQQYDNLLFTFGPGTKVDVK | | | | | | | | | 0998-x) |
| CoV2- | QSALTQPASVSGSPGQSITISCTGTSGD | IGHV3-30 | IGHJ4 | IGLV2-14 | IGLJ3 | 2343 | ARDTATYVLL | 3545 | SSYTSSSTL | Seth Zost et al., 2020 |
| 2009 | VGAYNYVSWYQQHPVKAPKLMIYDV | (Human) | (Human) | (Human) | (Human) | | WSGDFNLDY | | WV | (https://www.nature.co |
| | SKRPSGVSNRFSGSKSGNTASLTISGLQ | | | | | | | | | m/articles/s41591-020- |
| | TEDEADYYCSSYTSSSTLWVFGGGTKL | | | | | | | | | 0998-x) |
| | TVL | | | | | | | | | |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV1, | | | SARS-CoV2 | S; non- | B-cells; SARS-CoV2 | 256 | QVQLVESGGGVVQPGRSLRLSCAASGF | 1335 |
| 2011 | | SARS-CoV2 | | | | RBD | Human Patient | | TFSTYAMHWVRQAPGKGLEWVTLISYD | |
| | | | | | | | | | GGNKYYADSVKGRFTISRDNSKNTLYLQ | |
| | | | | | | | | | MNSLRAEDAAVYFCARGHTGNYYYGM | |
| | | | | | | | | | DVWGQGTTVTVSS | |
| CoV2- | Ab | SARS-CoV1, | | | SARS-CoV2 | S; non- | B-cells; SARS-CoV2 | 257 | QVHLVESGGGVVQPGRSLRLSCAASGF | 1336 |
| 2013 | | SARS-CoV2 | | | | RBD | Human Patient | | TFSNYGMHWVRQAPGKGLEWVAVISG | |
| | | | | | | | | | DENNKFYANSVKGRFTISRDNSKNTLSL | |
| | | | | | | | | | QMNSLRPEDTARYYCAKGGDSSGWAW | |
| | | | | | | | | | DGDNPPTDYWGQGTLVIVSS | |
| CoV2- | Ab | SARS-CoV1, | | SARS-CoV2 | | S; RBD | B-cells; SARS-CoV2 | 258 | EVQLVESGGGLVQPGRSLRLSCAASGFT | 1337 |
| 2015 | | SARS-CoV2 | | (weak) | | | Human Patient | | FDDYAMHWVRQAPGKGLEWVSGISW | |
| | | | | | | | | | NSGSIGYADSVKGRFTISRDNAKNSLYLQ | |
| | | | | | | | | | MNSLRTEDTALYYCAMGPFGELLPYYFD | |
| | | | | | | | | | YWGQGTLVTVSS | |
| CoV2- | Ab | SARS-CoV1, | | | SARS-CoV2 | S; RBD | B-cells; SARS-CoV2 | 259 | QVQLVQSGAEVKKPGASVKVSCKVSGY | 1338 |
| 2016 | | SARS-CoV2 | | | | | Human Patient | | TLTELSIHWVRQAPGKGLEWMGGFDP | |
| | | | | | | | | | EDAETIYAQNFQGRVTMTEDTSTDTAY | |
| | | | | | | | | | MELSSLRSEDTALYYCAAAPAVMTAGW | |
| | | | | | | | | | FDPWGQGTLVSVSS | |
| CoV2- | Ab | SARS-CoV1, | | | SARS-CoV2 | S; non- | B-cells; SARS-CoV2 | 260 | QVQLEESGPGLVKPSETLSLTCTVSGGSI | 1339 |
| 2017 | | SARS-CoV2 | | | | RBD | Human Patient | | SSHYWSWIRQPPGKGLEWIGYIQDSGS | |
| | | | | | | | | | TNYNPSLKSRVTISVDTSKNQFSLRLSSVT | |
| | | | | | | | | | TADTAVYYCVRGAMAWFDPWGQGTL | |
| | | | | | | | | | VTVSS | |
| CoV2- | Ab | SARS-CoV2 | SARS- | | SARS-CoV1, | S; NTD | B-cells; SARS-CoV2 | 261 | QVQLVQSGAEVKKPGASVKVSCKVSGY | 1340 |
| 2021 | | | CoV1 | | SARS-CoV2 | | Human Patient | | TLIELSIHWVRQAPGKGLEWMGGFDPE | |
| | | | | | | | | | DVETIYAQKFQGRVTMTEDTSTDTAYM | |
| | | | | | | | | | ELSSLTSEDRAVYYCATQPAAIGGTPPYY | |
| | | | | | | | | | WGQGTLVTVSS | |
| CoV2- | Ab | SARS-CoV2 | SARS- | | SARS-CoV1, | S; non- | B-cells; SARS-CoV2 | 262 | QVQLVQSGAEVKKPGASVKVSCKASGY | 1341 |
| 2022 | | | CoV1 | | SARS-CoV2 | RBD | Human Patient | | TFTSYVISWVRQAPGQGLEWMGWISA | |
| | | | | | | | | | YNGNTNYAQKLQGRVTMTTDTSTSTAY | |
| | | | | | | | | | MELRSLRSDDTAVYYCARDQGPTYYYGS | |
| | | | | | | | | | GSPHYGMDVWGQGTTVTVSS | |
| CoV2- | Ab | SARS-CoV1, | | | SARS-CoV2 | S; non- | B-cells; SARS-CoV2 | 263 | QVHLVESGGGVVQPGRSLRLSCAASGF | 1342 |
| 2025 | | SARS-CoV2 | | | | RBD | Human Patient | | TFSNYGMHWVRQAPGKGLEWVAVISG | |
| | | | | | | | | | DENNKFYANSVKGRFTISRDNSKNTLSL | |
| | | | | | | | | | QMNSLRPEDTARYYCAKGGDSSGWAW | |
| | | | | | | | | | DGDNPPTDYWGQGTLVIVSS | |
| CoV2- | Ab | SARS-CoV2 | SARS- | | SARS-CoV1, | S; NTD | B-cells; SARS-CoV2 | 264 | QVQLVQSGAEVKKPGASVKVSCKVSGY | 1343 |
| 2026 | | | CoV1 | | SARS-CoV2 | | Human Patient | | TLTELSIHWVRQAPGKGLEWMGGFDP | |
| | | | | | | | | | EDGETVYAQKFQGRVTMTEDTSSDTAY | |
| | | | | | | | | | MELSSLRSEDTAVYYCATSFPIRGDPSYY | |
| | | | | | | | | | YYYYGMDVWGQGTTVTVSS | |
| CoV2- | Ab | SARS-CoV2 | SARS- | | SARS-CoV1, | S; non- | B-cells; SARS-CoV2 | 265 | QVQLVESGGGVVQPGRSLRLSCAASGFI | 1344 |
| 2027 | | | CoV1 | | SARS-CoV2 | RBD | Human Patient | | FSTYGMHWVRQAPGKGLEWVAVISYD | |
| | | | | | | | | | GSNKYNADSVKGRFTISRDNSKNTLYLQ | |
| | | | | | | | | | MNSLRVEDTAVYYCAIYGYYYYGLDVW | |
| | | | | | | | | | GQGTTVTVSS | |
| CoV2- | Ab | SARS-CoV1, | | | SARS-CoV2 | S; non- | B-cells; SARS-CoV2 | 266 | QVHLVESGGGVVQPGRSLRLSCAASGF | 1345 |
| 2028 | | SARS-CoV2 | | | | RBD | Human Patient | | SFRNYGMHWVRQAPGKGLEWVAVISG | |
| | | | | | | | | | DENNKFYANSVKGRFTISRDNSKNTLSL | |
| | | | | | | | | | QMNSLRPEDTARYYCAKGGDSSGWAW | |
| | | | | | | | | | DGDNPPTDYWGQGTLVIVSS | |
| CoV2- | Ab | SARS-CoV1, | | | SARS-CoV2 | S; non- | B-cells; SARS-CoV2 | 267 | QVQLQESGPGLVKPSGTLSLTCVVSGGS | 1346 |
| 2029 | | SARS-CoV2 | | | | RBD | Human Patient | | ISSSNWGWVRQPPGKGLEWIGEIYLSG | |
| | | | | | | | | | TTNYNPSLTSRVTISVDKSKNQFSLKLNS | |
| | | | | | | | | | VTAADTAIYYCARPTAGAGGAFDIWGQ | |
| | | | | | | | | | GTVVTVSS | |
| CoV2- | Ab | SARS-CoV1, | | | SARS-CoV2 | S; RBD | B-cells; SARS-CoV2 | 268 | QVTLKESGPVLVKPTETLTLTCTVSGFSLS | 1347 |
| 2031 | | SARS-CoV2 | | | | | Human Patient | | NARMGVSWIRQPPGKALEWLAHIFWN | |
| | | | | | | | | | DENSYSTSLKTRLTISKDTSKSQVVLNMT | |
| | | | | | | | | | NMDPVDTATYYCARTEWLLSDNWFDS | |
| | | | | | | | | | WGQGTLVTVSS | |
| CoV2- | Ab | SARS-CoV1, | | | SARS-CoV2 | S; RBD | B-cells; SARS-CoV2 | 269 | QVQLVQSGAEVKKPGASVKVSCKASGD | 1348 |
| 2032 | | SARS-CoV2 | | | | | Human Patient | | TFSSYYLHWVRQAPGQGLQWMGISNP | |
| | | | | | | | | | SGGSTTYAQKFQGRVTMTGDTSTSTVY | |
| | | | | | | | | | MELSSLRSEDTAVYYCARGGLVPAARNA | |
| | | | | | | | | | FDIWGQGTMVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | ITCRASQSISSYLNWYQQKPGKAPKLLI | IGHV3-30- | IGHJ6 | IGKV1-39 | IGKJ3 | 2344 | ARGHTGNYYY | 3546 | QQSYSTFT | Seth Zost et al., 2020 |
| 2011 | YAASSLQSGVPSRFGGSASGTDFTLTIS | 3 | (Human) | (Human) | (Human) | | GMDV | | | (https://www.nature.co |
| | SLQPEDFATYYCQQSYSTFTFGPGTKV | (Human) | | | | | | | | m/articles/s41591-020- |
| | DIK | | | | | | | | | 0998-x) |
| CoV2- | DIVMTQSPDFLAVSLGERATINCKSSQ | IGHV3-30 | IGHJ4 | IGKV4-1 | IGKJ4 | 2345 | AKGGDSSGW | 3547 | QQYYTAPL | Seth Zost et al., 2020 |
| 2013 | SVLHTPKNKNYLAWYKQKPGQPPKVL | (Human) | (Human) | (Human) | (Human) | | AWDGDNPPT | | T | (https://www.nature.co |
| | IYWASTRESGVPERFSGSGSGTDFTLII | | | | | | DY | | | m/articles/s41591-020- |
| | SSLQAEDAAVYYCQQYYTAPLTFGGGT | | | | | | | | | 0998-x) |
| | KVEIK | | | | | | | | | |
| CoV2- | EIVLTQSPATLSLSPGERATLSCRASQS | IGHV3-9 | IGHJ4 | IGKV3-11 | IGKJ2 | 2346 | AMGPFGELLP | 3548 | QQRSNWP | Seth Zost et al., 2020 |
| 2015 | VSSYLAWYQQKPGQAPRLLIYDTSNR | (Human) | (Human) | (Human) | (Human) | | YYFDY | | PYT | (https://www.nature.co |
| | ATGIPARFSGSGSGTDFTLTISSLEPED | | | | | | | | | m/articles/s41591-020- |
| | FAVYYCQQRSNWPPYTFGQGTKLEIK | | | | | | | | | 0998-x) |
| CoV2- | QSALTQPASVSGSPGQSITISCTGTSSD | IGHV1-24 | IGHJ5 | IGLV2-14 | IGLJ3 | 2347 | AAAPAVMTA | 3549 | SSYTSSTT | Seth Zost et al., 2020 |
| 2016 | VGGYHYVSWYQHHPGKAPKLIIYDVIK | (Human) | (Human) | (Human) | (Human) | | GWFDP | | WV | (https://www.nature.co |
| | RPSGVSNRFSGSKSGNTASLTISGLQA | | | | | | | | | m/articles/s41591-020- |
| | EDEAYYYCSSYTSSTTWVFGGGTRLTV | | | | | | | | | 0998-x) |
| | L | | | | | | | | | |
| CoV2- | QSVLTQPPSVSGAPGQRVTISCTGSSS | IGHV4-59 | IGHJ5 | IGLV1-40 | IGLJ1 | 2348 | VRGAMAWF | 3550 | QSFDSSLS | Seth Zost et al., 2020 |
| 2017 | NIGAGYNVHWYQQLPGTAPKLLIYGN | (Human) | (Human) | (Human) | (Human) | | DP | | GSDV | (https://www.nature.co |
| | NNRPSGVPDRFSGSKSGTSASLAITGL | | | | | | | | | m/articles/s41591-020- |
| | QAEDEADYYCQSFDSSLSGSDVFGTGT | | | | | | | | | 0998-x) |
| | KVSVL | | | | | | | | | |
| CoV2- | QSVLTQPPSASGTPGQRVTISCSGSSS | IGHV1-24 | IGHJ4 | IGLV1-47 | IGLJ2 | 2349 | ATQPAAIGGT | 3551 | AAWDASL | Seth Zost et al., 2020 |
| 2021 | NIGSNYVYWYQQLPGTAPKLLIYRNN | (Human) | (Human) | (Human) | (Human) | | PPYY | | SGHVV | (https://www.nature.co |
| | QRPSGVPDRFSGSKSGTSASLAISGLRS | | | | | | | | | m/articles/s41591-020- |
| | EDEADYYCAAWDASLSGHVVFGGGT | | | | | | | | | 0998-x) |
| | KLTVL | | | | | | | | | |
| CoV2- | DIVMAQTPLSLPVTPGEPASISCRSSQS | IGHV1-18 | IGHJ6 | IGKV2-40 | IGKJ2 | 2350 | ARDQGPTYYY | 3552 | MQRIEFP | Seth Zost et al., 2020 |
| 2022 | LLDSDDGNTYFDWYLQKPGQSPQLLI | (Human) | (Human) | (Human) | (Human) | | GSGSPHYGM | | WT | (https://www.nature.co |
| | YTLSYRASGVPDRFSGSGSGTDFTLKIN | | | | | | DV | | | m/articles/s41591-020- |
| | RVEAEDVGVYYCMQRIEFPWTFGQG | | | | | | | | | 0998-x) |
| | TKLEIK | | | | | | | | | |
| CoV2- | DIVMTQSPDFLAVSLGERATINCKSSQ | IGHV3-30 | IGHJ4 | IGKV4-1 | IGKJ4 | 2351 | AKGGDSSGW | 3553 | QQYYTAPL | Seth Zost et al., 2020 |
| 2025 | SVLHTPKNKNYLAWYKQKPGQPPKVL | (Human) | (Human) | (Human) | (Human) | | AWDGDNPPT | | T | (https://www.nature.co |
| | IYWASTRESGVPERFSGSGSGTDFTLII | | | | | | DY | | | m/articles/s41591-020- |
| | SSLQAEDAAVYYCQQYYTAPLTFGGGT | | | | | | | | | 0998-x) |
| | KVEIK | | | | | | | | | |
| CoV2- | DIQMTQSPSSLSASVGDRVTITCRASQ | IGHV1-24 | IGHJ6 | IGKV1-16 | IGKJ1 | 2352 | ATSFPIRGDPS | 3554 | QQYNSYP | Seth Zost et al., 2020 |
| 2026 | GITNYLAWFQQKPGKAPKSLIYAVSSL | (Human) | (Human) | (Human) | (Human) | | YYYYYYGMDV | | WT | (https://www.nature.co |
| | QSGVPSKFSGSGSGTDFTLTISSLQPED | | | | | | | | | m/articles/s41591-020- |
| | FATYYCQQYNSYPWTFGQGTKVEIK | | | | | | | | | 0998-x) |
| CoV2- | SYELTQPPSVSVSPGQTARITCSGDALP | IGHV3-30 | IGHJ6 | IGLV3-25 | IGLJ3 | 2353 | AIYGYYYYGLD | 3555 | QSADSSGT | Seth Zost et al., 2020 |
| 2027 | KQYAYWYQQKPGQAPVLVIYKDSERP | (Human) | (Human) | (Human) | (Human) | | V | | YFWV | (https://www.nature.co |
| | SGIPERFSGSSSGTTVTLTISGVQAEDE | | | | | | | | | m/articles/s41591-020- |
| | ADYYCQSADSSGTYFWVFGGGTKLTV | | | | | | | | | 0998-x) |
| | L | | | | | | | | | |
| CoV2- | DIVMTQSPDFLAVSLGERATINCKSSQ | IGHV3-30 | IGHJ4 | IGKV4-1 | IGKJ4 | 2354 | AKGGDSSGW | 3556 | QQYYTAPL | Seth Zost et al., 2020 |
| 2028 | SVLHTPKNKNYLAWYKQKPGQPPKVL | (Human) | (Human) | (Human) | (Human) | | AWDGDNPPT | | T | (https://www.nature.co |
| | IYWASTRESGVPERFSGSGSGTDFTLII | | | | | | DY | | | m/articles/s41591-020- |
| | SSLQAEDAAVYYCQQYYTAPLTFGGGT | | | | | | | | | 0998-x) |
| | KVEIK | | | | | | | | | |
| CoV2- | QSVLTQPPSVSGTPGQRVTISCSGSNS | IGHV4-4 | IGHJ3 | IGLV1-44 | IGLJ3 | 2355 | ARPTAGAGG | 3557 | SVWDDSL | Seth Zost et al., 2020 |
| 2029 | KIGSYSVNWYQQLPGTAPKLLIYRNNQ | (Human) | (Human) | (Human) | (Human) | | AFDI | | NGPL | (https://www.nature.co |
| | RPSGVPDRFFGSKSGTSASLAISGLQSE | | | | | | | | | m/articles/s41591-020- |
| | DEADYYCSVWDDSLNGPLFGGGTKLT | | | | | | | | | 0998-x) |
| | FV | | | | | | | | | |
| CoV2- | SYELTQPPSVSVSPGQTARITCSGDAW | IGHV2-26 | IGHJ5 | IGLV3-25 | IGLJ3 | 2356 | ARTEWLLSDN | 3558 | QSSDSSGV | Seth Zost et al., 2020 |
| 2031 | PNQYAYWYQQKPGQAPVLVIYKDTER | (Human) | (Human) | (Human) | (Human) | | WFDS | | V | (https://www.nature.co |
| | PSGIPERFSGSSSGTTVTLTISGVQAED | | | | | | | | | m/articles/s41591-020- |
| | EADYFCQSSDSSGVVFGGGTKLTVL | | | | | | | | | 0998-x) |
| CoV2- | DIQMTQSPSTLSASVGDRVTITCRASQ | IGHV1-46 | IGHJ3 | IGKV1-5 | IGKJ1 | 2357 | ARGGLVPAA | 3559 | QQYNSYS | Seth Zost et al., 2020 |
| 2032 | SISSWLAWYQQKPGKAPKLLIYKASSL | (Human) | (Human) | (Human) | (Human) | | RNAFDI | | WT | (https://www.nature.co |
| | ESGVPSRFSGSGSGTEFTLTISSLQPDD | | | | | | | | | m/articles/s41591-020- |
| | FATYYCQQYNSYSWTFGQGTKVEIK | | | | | | | | | 0998-x) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV1, | | | SARS-CoV2 | S; non- | B-cells; SARS-CoV2 | 270 | QVQLQESGPGLVKPSGTLSLTCVVSGGS | 1349 |
| 2033 | | SARS-CoV2 | | | | RBD | Human Patient | | ISSSNWGWVRQPPGKGLEWIGEIYHSG | |
| | | | | | | | | | TTNYNPSLRSRVTISVDKSKNQLSLKLNS | |
| | | | | | | | | | VTAADTAIYYCARPTAGAGGAFDTWGQ | |
| | | | | | | | | | GTMVTVSA | |
| CoV2- | Ab | SARS-CoV1, | | | SARS-CoV2 | S; non- | B-cells; SARS-CoV2 | 271 | QVQLVQSGAEVKKPGASVKVSCKASGY | 1350 |
| 2034 | | SARS-CoV2 | | | | RBD | Human Patient | | TFTTYGISWVRQAPGQGLEWMGWISA | |
| | | | | | | | | | YNGNSNYGKKFQGRVTMTADTSTSTAY | |
| | | | | | | | | | MELRSLRSDDTAVYYCARDLPIKVVVPA | |
| | | | | | | | | | ADYNWFDPWGQGTLVTVSS | |
| CoV2- | Ab | SARS-CoV2 | SARS- | | SARS-CoV1, | S; non- | B-cells; SARS-CoV2 | 272 | QITLKESGPTLVKPTQTLTLTCTFSGFSLN | 1351 |
| 2035 | | | CoV1 | | SARS-CoV2 | RBD | Human Patient | | TNGVAVGWIRQPPGKALEWLALIYWD | |
| | | | | | | | | | DDKRYSPSLKSRLTITKDTSKNQVVLTMT | |
| | | | | | | | | | NMDPVDTATYYCAHRRGILTEDAFDIW | |
| | | | | | | | | | GQGTMVTVSS | |
| CoV2- | Ab | SARS-CoV2 | SARS- | SARS-CoV2 | | S; RBD | B-cells; SARS-CoV2 | 273 | EVQLVESGGGLVQPGESLRLSCAASGIT | 1352 |
| 2037 | | | CoV1 | | | | Human Patient | | VSSNYMSWVRQAPGKGLEWVSVIYSG | |
| | | | | | | | | | GSTFYADSVKGRFIISRHNSKNTLYLQM | |
| | | | | | | | | | NSLRAEDTAVYYCARDLNEHGLDVWG | |
| | | | | | | | | | QGTTVSVSS | |
| CoV2- | Ab | SARS-CoV2 | SARS- | | SARS-CoV1, | S; RBD | B-cells; SARS-CoV2 | 274 | QVQLVQSGAEVKKPGASVKVSCKASGY | 1353 |
| 2039 | | | CoV1 | | SARS-CoV2 | | Human Patient | | TFTSYYMHWVRQAPGQGLEWMGIINP | |
| | | | | | | | | | SAGSTSYAQKFQGRVTMTRDTSTSTVY | |
| | | | | | | | | | MELSSLRSEDTAVYYCARGTLIPAHRGAF | |
| | | | | | | | | | DIWGQGTMVTVSS | |
| CoV2- | Ab | SARS-CoV1, | | | SARS-CoV2 | S; non- | B-cells; SARS-CoV2 | 275 | QVQLQESGPGLVKPSQTLSLTCTVSGGSI | 1354 |
| 2041 | | SARS-CoV2 | | | | RBD | Human Patient | | SSGTYYWSWIRQPAGKGLEWIGRFYTS | |
| | | | | | | | | | GSTNYNPSLKSRVTISVDASKNQFSLKLS | |
| | | | | | | | | | SVTAADTAVYYCARARPDYYYYYAMDV | |
| | | | | | | | | | WGQGTTVTVSS | |
| CoV2- | Ab | SARS-CoV2 | SARS- | SARS-CoV2 | | S; RBD | B-cells; SARS-CoV2 | 276 | EVQLVESGGGLVQPGRSLRLSCAASGFT | 1355 |
| 2046 | | | CoV1 | | | | Human Patient | | FDDYAMHWVRQAPGKGLEWVSGISW | |
| | | | | | | | | | NSGSIAYTDSVKGRFTISRDNAKNSLYLQ | |
| | | | | | | | | | MNSLRAEDTALYYCAKAHSTGHQYYYG | |
| | | | | | | | | | MDVWGQGTTVTVSS | |
| CoV2- | Ab | SARS-CoV2 | SARS- | SARS-CoV2 | | S; RBD | B-cells; SARS-CoV2 | 277 | QVQLVQSGAEVKKPGASVKVSCKASGY | 1356 |
| 2050 | | | CoV1 | | | | Human Patient | | TFTDYYMHWVRQAPGQGLEWMGWI | |
| | | | | | | | | | NPNSRGTNYAQKFQGRVTMTRDTSIST | |
| | | | | | | | | | VYMELSRLTSDDTAVYYCARVVVLGYGR | |
| | | | | | | | | | PNNYYDGRNVWDYWGQGTLVTVSS | |
| CoV2- | Ab | SARS-CoV2 | SARS- | | SARS-CoV1, | S; RBD | B-cells; SARS-CoV2 | 278 | QVTLRESGPALVKPTQTLSLTCTFSGFSL | 1357 |
| 2051 | | | CoV1 | | SARS-CoV2 | | Human Patient | | GTSGMCVSWIRQPPGKALEWLARIDW | |
| | | | | | | | | | DDDKYYSTSLKTRLTISKDTSKNQVVLTM | |
| | | | | | | | | | TNMDPVDTATYYCARGVVTYDYWGQG | |
| | | | | | | | | | TLVTVSS | |
| CoV2- | Ab | SARS-CoV2 | SARS- | | SARS-CoV1, | S; non- | B-cells; SARS-CoV2 | 279 | QVQLVQSGAEVKKPGSSVKVSCKASGD | 1358 |
| 2054 | | | CoV1 | | SARS-CoV2 | RBD | Human Patient | | TFSSYTINWVRQAPGQGLEWMGRIIPIL | |
| | | | | | | | | | GIPNYAQKFQGRVTITADKSTSTAFMEL | |
| | | | | | | | | | SSLRSEDTAVYYCARGRGYSNYGASYYM | |
| | | | | | | | | | DVWGKGTTVTVSS | |
| CoV2- | Ab | SARS-CoV1, | | | SARS-CoV2 | S; RBD | B-cells; SARS-CoV2 | 280 | EVQLVESGGGLVQPGRSLRLSCAASGFT | 1359 |
| 2055 | | SARS-CoV2 | | | | | Human Patient | | FDDYAMNWVRQPPGKGLEWVSGISW | |
| | | | | | | | | | NSDSIGYADSVKGRFTISRDNAKNSLYLQ | |
| | | | | | | | | | MNSLRAEDTAMYYCAKGRGAGYTSYM | |
| | | | | | | | | | DVWGKGTTVTVSS | |
| CoV2- | Ab | SARS-CoV2 | SARS- | SARS-CoV2 | | S; RBD | B-cells; SARS-CoV2 | 281 | QVQLAQSGAEVKKPGASVKVSCKAAGY | 1360 |
| 2064 | | | CoV1 | | | | Human Patient | | TFTSYDINWVRQATGQGLEWMGWMN | |
| | | | | | | | | | SNSGNAGYAQKFQGRVTMTRDTSTSTA | |
| | | | | | | | | | YMELSSLTSDDTAVYYCARMRTGWPTH | |
| | | | | | | | | | GRPDDFWGRGTLVTVSS | |
| CoV2- | Ab | SARS-CoV2 | SARS- | SARS-CoV2 | | S; RBD | B-cells; SARS-CoV2 | 282 | EVQLVESGGGLVQPGGSLRLSCAASGFT | 1361 |
| 2068 | | | CoV1 | | | | Human Patient | | VSSNYMSWVRQAPGKGLEWVSVIYPG | |
| | | | | | | | | | GSAFYADSVKGRFTISRHNSNNTLCLQM | |
| | | | | | | | | | NSLRTEDTAVYYCARSYDILTGYRDAFDI | |
| | | | | | | | | | WGQGTMVTVSS | |
| CoV2- | Ab | SARS-CoV2 | SARS- | | SARS-CoV1, | S; RBD | B-cells; SARS-CoV2 | 283 | QVQLQQWGAGLLKPSETLSLTCAVSGG | 1362 |
| 2070 | | | CoV1 | | SARS-CoV2 | | Human Patient | | SFSAYYWSWIRQPPGKGLEWIGEINHS | |
| | | | | | | | | | GSTNYNPSLRSRVTISVDTSKNQFSLKLSS | |
| | | | | | | | | | VTAADTAVYYCARVGYSQGYYYYYMDV | |
| | | | | | | | | | WGKGTTVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | QSVLTQPPSASGTPGQRVTISCSGSNS | IGHV4-4 | IGHJ3 | IGLV1-44 | IGLJ3 | 2358 | ARPTAGAGG | 3560 | SVWDDSL | Seth Zost et al., 2020 |
| 2033 | KIGSYSVNWYQQLPGTAPKLLIYRNNQ | (Human) | (Human) | (Human) | (Human) | | AFDT | | NGPL | (https://www.nature.co |
| | RPSGVPDRFSGSKSGTSASLAISGLQSE | | | | | | | | | m/articles/s41591-020- |
| | DEADYYCSVWDDSLNGPLFGGGTKLT | | | | | | | | | 0998-x) |
| | FL | | | | | | | | | |
| CoV2- | DIQMTQSPSSLSASVGDRVTITCRASQ | IGHV1-18 | IGHJ5 | IGKV1-39 | IGKJ4 | 2359 | ARDLPIKVV | 3561 | QQSYSTPP | Seth Zost et al., 2020 |
| 2034 | SISSYLNWYQQKPGKAPKLLIYAASSLQ | (Human) | (Human) | (Human) | (Human) | | VPAADYNWF | | T | (https://www.nature.co |
| | SGVPSRFSGSGSGTDFTLTISSLQPEDF | | | | | | DP | | | m/articles/s41591-020- |
| | ATYYCQQSYSTPPTFGGGTKVQIK | | | | | | | | | 0998-x) |
| CoV2- | DIQMTQSPSSLSASVGDRVTITCRASQ | IGHV2-5 | IGHJ3 | IGKV1-39 | IGKJ1 | 2360 | AHRRGILTE | 3562 | QQSYNTPR | Seth Zost et al., 2020 |
| 2035 | SISSYLNWYQQRPGKAPKLLIYAASSLQ | (Human) | (Human) | (Human) | (Human) | | DAFDI | | T | (https://www.nature.co |
| | SGVPSRFSGSGYGTDFTLTISSLQPEDF | | | | | | | | | m/articles/s41591-020- |
| | ATYYCQQSYNTPRTFGQGTKVEIK | | | | | | | | | 0998-x) |
| CoV2- | DIQMTQSPSSVSASVGDRVTITCRASQ | IGHV3-53 | IGHJ6 | IGKV1-12 | IGKJ5 | 2361 | ARDLNEHGL | 3563 | QQTNSFPT | Seth Zost et al., 2020 |
| 2037 | GISSWLAWYQQKPGKAPKLLIYAASSL | (Human) | (Human) | (Human) | (Human) | | DV | | | (https://www.nature.co |
| | QSGVPSRFSGSGSGTDFTLTISSLQPED | | | | | | | | | m/articles/s41591-020- |
| | FATYYCQQTNSFPTFGQGTRLEIR | | | | | | | | | 0998-x) |
| CoV2- | EIVLTQSPGTLSLSPGERATLSCRASQS | IGHV1-46 | IGHJ3 | IGKV3-20 | IGKJ3 | 2362 | ARGTLIPAH | 3564 | QQYGNSP | Seth Zost et al., 2020 |
| 2039 | VSSSFLAWYQQKPGQAPRLLIYGASN | (Human) | (Human) | (Human) | (Human) | | RGAFDI | | Q | (https://www.nature.co |
| | RATGIPDRFSGSGSGTDFTLTISRLEPE | | | | | | | | | m/articles/s41591-020- |
| | DFAVYYCQQYGNSPQFGPGTKVDIK | | | | | | | | | 0998-x) |
| CoV2- | DIQMTQSPSSLSASVGDRVTITCRASQ | IGHV4-61 | IGHJ6 | IGKV1-5 | IGKJ1 | 2363 | ARARPDYYY | 3565 | QQYNSYST | Seth Zost et al., 2020 |
| 2041 | SISNWLAWYQQKPGKAPKLLIYKASSL | (Human) | (Human) | (Human) | (Human) | | YYAMDV | | WT | (https://www.nature.co |
| | ESGVPSRFSGSGSGTEFTLTISSLQPDD | | | | | | | | | m/articles/s41591-020- |
| | FATYYCQQYNSYSTWTFGQGTKVEIK | | | | | | | | | 0998-x) |
| CoV2- | DIQMTQSPSSLSASVGDRVTITCRASQ | IGHV3-9 | IGHJ6 | IGKV1-39 | IGKJ2 | 2364 | AKAHSTGHQ | 3566 | QQSYNTPY | Seth Zost et al., 2020 |
| 2046 | SISSFLNWYQQKPGKAPKLLIYAAFNL | (Human) | (Human) | (Human) | (Human) | | YYYGMDV | | T | (https://www.nature.co |
| | QSGVPSRFSGSGSGTDFTLTISSLQSED | | | | | | | | | m/articles/s41591-020- |
| | FATYYCQQSYNTPYTFGQGTKLEIK | | | | | | | | | 0998-x) |
| CoV2- | QSVLTQPPSASGTPGQRVIISCSGSSSN | IGHV1-2 | IGHJ4 | IGLV1-44 | IGLJ2 | 2365 | ARVVVLGYG | 3567 | AAWDDSL | Seth Zost et al., 2020 |
| 2050 | IGSNTVKWYHQLPGTAPKLLICSNNQR | (Human) | (Human) | (Human) | (Human) | | RPNNYYDGR | | NALV | (https://www.nature.co |
| | PSGVPDRFSGSKSDTSASLAISGLQSED | | | | | | NVWDY | | | m/articles/s41591-020- |
| | EADYYCAAWDDSLNALVFGGGTKLTV | | | | | | | | | 0998-x) |
| | L | | | | | | | | | |
| CoV2- | DIQMTQSPSSLSASVGDRVTITCRASQ | IGHV2-70 | IGHJ4 | IGKV1-39 | IGKJ5 | 2366 | ARGVVTYDY | 3568 | QQSYSTPG | Seth Zost et al., 2020 |
| 2051 | SIAGYLNWYQQKPGKAPKLLIYGTTSL | (Human) | (Human) | (Human) | (Human) | | | | T | (https://www.nature.co |
| | QSGVPVRFSGSGSGTDFTLTISSLQPE | | | | | | | | | m/articles/s41591-020- |
| | DFATYYCQQSYSTPGTFGQGTRLEIK | | | | | | | | | 0998-x) |
| CoV2- | DIQMTQSPSSLSASVGDRVTITCQASQ | IGHV1-69 | IGHJ6 | IGKV1-33 | IGKJ2 | 2367 | ARGRGYSNY | 3569 | QQSDNLP | Seth Zost et al., 2020 |
| 2054 | DINHYLNWYQQKPGKAPKLLIYDASN | (Human) | (Human) | (Human) | (Human) | | GASYYMDV | | MYT | (https://www.nature.co |
| | LETGVPSRFSGSGSGTDFTFTITSLQPE | | | | | | | | | m/articles/s41591-020- |
| | DVATYYCQQSDNLPMYTFGQGTKLEI | | | | | | | | | 0998-x) |
| | K | | | | | | | | | |
| CoV2- | SYVLTQPPSVSVAPGKTARITCEGNNI | IGHV3-9 | IGHJ6 | IGLV3-21 | IGLJ2 | 2368 | AKGRGAGYTS | 3570 | QVWDSSS | Seth Zost et al., 2020 |
| 2055 | GSKSVHWYQQKPGQAPVLVVYDDSG | (Human) | (Human) | (Human) | (Human) | | YMDV | | DHHVV | (https://www.nature.co |
| | RPSGIPERFSGSNSGNTATLTISRVEAG | | | | | | | | | m/articles/s41591-020- |
| | DEADYFCQVWDSSSDHHVVFGGGTK | | | | | | | | | 0998-x) |
| | LTVL | | | | | | | | | |
| CoV2- | QSVLTQPPSASGTPGQRVTISCSGSNS | IGHV1-8 | IGHJ4 | IGLV1-44 | IGLJ2 | 2369 | ARMRTGWPT | 3571 | LVWDDSL | Seth Zost et al., 2020 |
| 2064 | NIGSYTVNWYQQFPGTAPKLLIYDNN | (Human) | (Human) | (Human) | (Human) | | HGRPDDF | | NGLV | (https://www.nature.co |
| | QRTSGVPDRFSGSKSGTSASLAISGLQ | | | | | | | | | m/articles/s41591-020- |
| | SEDEANYYCLVWDDSLNGLVFGGGTK | | | | | | | | | 0998-x) |
| | LTVL | | | | | | | | | |
| CoV2- | QSVLTQPPSVSGAPGQRVTISCTGSSS | IGHV3-53 | IGHJ3 | IGLV1-40 | IGLJ2 | 2370 | ARSYDILTG | 3572 | QSYDSRLS | Seth Zost et al., 2020 |
| 2068 | NIGSGSDVHWYQQLPGTAPKLLIYGN | (Human) | (Human) | (Human) | (Human) | | YRDAFDI | | GFVV | (https://www.nature.co |
| | TNRPSGVPDRFSGSKSGTSASLAITGL | | | | | | | | | m/articles/s41591-020- |
| | QAEDEADYYCQSYDSRLSGFVVFGGG | | | | | | | | | 0998-x) |
| | TKLTVL | | | | | | | | | |
| CoV2- | DIQMTQSPSSLSASVGDRVTITCRASQ | IGHV4-34 | IGHJ6 | IGKV1-39 | IGKJ4 | 2371 | ARVGYSQGY | 3573 | QQSYTTLL | Seth Zost et al., 2020 |
| 2070 | SISNYLNWYQQKPGKAPKLLIYAASSL | (Human) | (Human) | (Human) | (Human) | | YYYYMDV | | T | (https://www.nature.co |
| | QSGVPSRFSGSGSGTDFSLTISSLQPED | | | | | | | | | m/articles/s41591-020- |
| | FATYSCQQSYTTLLTFGGGTKVEIK | | | | | | | | | 0998-x) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV2 | SARS- | SARS-CoV2 | | S; RBD | B-cells; SARS-CoV2 | 284 | QMQLVQSGPEVKKPGTSVKVSCKTSGF | 1363 |
| 2072 | | | CoV1 | | | | Human Patient | | TFTSSAIQWVRQARGQRLEWIGWIVVG | |
| | | | | | | | | | SGNTNYAQKFQERVTITRDMSTSTAYM | |
| | | | | | | | | | ELSSLRSEDTAVYYCAAPHCNRTSCYDAF | |
| | | | | | | | | | DLWGQGTMVTVSS | |
| CoV2- | Ab | SARS-CoV2 | SARS- | | SARS-CoV1, | S; RBD | B-cells; SARS-CoV2 | 285 | QVQLVQSGAEVKKPGSSVKVSCKASGG | 1364 |
| 2078 | | | CoV1 | | SARS-CoV2 | | Human Patient | | TFSSYSITWVRQAPGQGLEWMGRIIPVL | |
| | | | | | | | | | GIANYAQKFQDRVTITADKSTSTAYMEL | |
| | | | | | | | | | SSLRSEDTAVYYCARVGVSGFKSGSNWY | |
| | | | | | | | | | FDLWGRGTLVTVSS | |
| CoV2- | Ab | SARS-CoV2 | SARS- | | SARS-CoV1, | S; RBD | B-cells; SARS-CoV2 | 286 | EVQLVESGGGLVQPGGSLRLSCAASGLT | 1365 |
| 2080 | | | CoV1 | | SARS-CoV2 | | Human Patient | | VRSNYMTWVRQTPGKGLEWVSVIYSG | |
| | | | | | | | | | GSTFYADSVKGRFTISRDNSKNTVYLQM | |
| | | | | | | | | | NSLRAEDTAVYYCARDLVTYGLDVWGQ | |
| | | | | | | | | | GTTVTVSS | |
| CoV2- | Ab | SARS-CoV1, | | | SARS-CoV2 | S; RBD | B-cells; SARS-CoV2 | 287 | EVQLVESGGGVVRPGGSLRLSCAASGFI | 1366 |
| 2082 | | SARS-CoV2 | | | | | Human Patient | | FDDYDMTWVRQAPGKGLEWVSGISW | |
| | | | | | | | | | NGGNTGYADSVKGRFTISRDNAKNSLYL | |
| | | | | | | | | | QMNSLRAEDTALYHCAVIMSPIPRYSGY | |
| | | | | | | | | | DWAGGAFDIWGQGTMVTVSS | |
| CoV2- | Ab | SARS-CoV1, | | | SARS-CoV2 | S; RBD | B-cells; SARS-CoV2 | 288 | QVQLVESGGGVVQPGRSLRLSCAASGF | 1367 |
| 2083 | | SARS-CoV2 | | | | | Human Patient | | TFSNYGMHWVRQAPGKGLEWVAVMS | |
| | | | | | | | | | YDGSNKYYADSVKGRFTISRDNSKNTLYL | |
| | | | | | | | | | QMNSLRAEDTAVYYCAKNLGPYCSGGT | |
| | | | | | | | | | CYSLVGDYWGQGTLVTVSS | |
| CoV2- | Ab | SARS-CoV1, | | | SARS-CoV2 | S; RBD | B-cells; SARS-CoV2 | 289 | EVQLVESGGGVVRPGGSLRLSCAASGFI | 1368 |
| 2094 | | SARS-CoV2 | | | | | Human Patient | | FDDYDMTWVRQAPGKGLEWVSGINW | |
| | | | | | | | | | NGGSTGYADSVKGRFTISRDNAKNSLYL | |
| | | | | | | | | | QMNSLRAEDTALYHCAVIMSPIPRYSGY | |
| | | | | | | | | | DWAGDAFDIWGQGTMVTVSS | |
| CoV2- | Ab | SARS-CoV2 | SARS- | SARS-CoV2 | | S; RBD | B-cells; SARS-CoV2 | 290 | QVQLVQSGAEVKKPGASVKVSCKASGY | 1369 |
| 2096 | | | CoV1 | | | | Human Patient | | TFGSFDINWVRQATGQGLEWMGRMN | |
| | | | | | | | | | SNSGNTAYAQKFQGRVTMTRDTSTNTA | |
| | | | | | | | | | YMELSSLRSEDTAMYYCARMRSGWPTH | |
| | | | | | | | | | GRPDDFWGRGTLVTVSS | |
| CoV2- | Ab | SARS-CoV2 | SARS- | SARS-CoV2 | | S; RBD | B-cells; SARS-CoV2 | 291 | EVQLVESGGGLVQPGRSLRLSCAASGFT | 1370 |
| 2097 | | | CoV1 | | | | Human Patient | | FDDYAMHWVRQAPGKGLEWVSGISW | |
| | | | | | | | | | NSGTIGYADSVKGRFIISRDNAKNSLYLQ | |
| | | | | | | | | | MNSLRPEDTALYYCAKDIIRQGEDGMD | |
| | | | | | | | | | VWGQGTTVTVSS | |
| CoV2- | Ab | SARS-CoV2 | SARS- | SARS-CoV2 | | S; RBD | B-cells; SARS-CoV2 | 292 | EVQLLESGGGLIQPGGSLRLSCAASGFTF | 1371 |
| 2098 | | | CoV1 | | | | Human Patient | | SNYAMSWVRQAPGKGLEWVSGIISTSG | |
| | | | | | | | | | GATYNADSVRGRFTTSRDNSKNILYLQM | |
| | | | | | | | | | NSLRGEDTAVYYCVKGLFDWFPLWGQ | |
| | | | | | | | | | GTMVTVSS | |
| CoV2- | Ab | SARS-CoV1, | | SARS-CoV2 | | S; RBD | B-cells; SARS-CoV2 | 293 | EVQLVESGGGLVQPGGSLRLSCAASGFT | 1372 |
| 2103 | | SARS-CoV2 | | | | | Human Patient | | FSRHWMTWVRQAPGKGLEWVANIKQ | |
| | | | | | | | | | DGSEKYYVDSVKGRLTISRDNAKNSLYL | |
| | | | | | | | | | QMNSLRAEDTAVYYCARLGFYYGGADY | |
| | | | | | | | | | WGQGTLVTVSS | |
| CoV2- | Ab | SARS-CoV2 | SARS- | | SARS-CoV1, | S; RBD | B-cells; SARS-CoV2 | 294 | NVQLVESGGGLVQPGGSLRLSCAASGF | 1373 |
| 2108 | | | CoV1 | | SARS-CoV2 | | Human Patient | | TFHHYAMHWVRQAPGKGLEWVSGISG | |
| | | | | | | | | | SSDYRAYADSLKGRFTISRDYAKNSLWL | |
| | | | | | | | | | QMNSLTSEDTAFYYCAKGVDYGGKLAYF | |
| | | | | | | | | | DSWGQGTLVTVSS | |
| CoV2- | Ab | SARS-CoV2 | SARS- | | SARS-CoV1, | S; RBD | B-cells; SARS-CoV2 | 295 | QVQLVESGGGVVQPGRSLRLSCAASGF | 1374 |
| 2110 | | | CoV1 | | SARS-CoV2 | | Human Patient | | SFSSYVMNWVRQAPGKGqLEWVAVISY | |
| | | | | | | | | | DGSSKYYADSVKGRFTISRDNSKNTLYLQ | |
| | | | | | | | | | MNSLRAEDTAVYYCARDIDSGYDPTPVF | |
| | | | | | | | | | DYWGQGTLVTVSS | |
| CoV2- | Ab | SARS-CoV1, | | | SARS-CoV2 | S; RBD | B-cells; SARS-CoV2 | 296 | EVQLVESGGGLVQPGGSLRLSCAASGFT | 1375 |
| 2111 | | SARS-CoV2 | | | | | Human Patient | | FSSYDLHWVRQGTGKRLEWVSAIGTAG | |
| | | | | | | | | | DTYYLGSVKGRFTISRENAKNSLYLQMN | |
| | | | | | | | | | SLRAGDTAVYYCARVLYDSSGFYNWFDP | |
| | | | | | | | | | WGQGTLVTVSS | |
| CoV2- | Ab | SARS-CoV2 | SARS- | SARS-CoV2 | | S; RBD | B-cells; SARS-CoV2 | 297 | EVQLVESGGGLIQPGGSLRLSCAASEVT | 1376 |
| 2113 | | | CoV1 | | | | Human Patient | | VSSNYMSWVRQAPGKGLEWVSLIYSGG | |
| | | | | | | | | | TTYYADSVKGRFTISRDNSKNTLYLQMN | |
| | | | | | | | | | SLRAEDTAVYYCARDFLRWHDLWGQG | |
| | | | | | | | | | TLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | EIVLTQSPGTLSLSPGERATLSCRASQS | IGHV1-58 | IGHJ3 | IGKV3-20 | IGKJ1 | 2372 | AAPHCNRTSC | 3574 | QQYGSSP | Seth Zost et al., 2020 |
| 2072 | VSSSYLGWYQQKPGQAPRLLIYGASSR | (Human) | (Human) | (Human) | (Human) | | YDAFDL | | WT | (https://www.nature.co |
| | ATGIPDRFSGSGSGTDFTLTISRLEPED | | | | | | | | | m/articles/s41591-020- |
| | FAVYYCQQYGSSPWTFGQGTKVEIK | | | | | | | | | 0998-x) |
| CoV2- | QSVLTQPPSVSGAPGQRVTLSCTGSNS | IGHV1-69 | IGHJ2 | IGLV1-40 | IGLJ3 | 2373 | ARVGVSGFKS | 3575 | QSYDSSLS | Seth Zost et al., 2020 |
| 2078 | NIGAGYDVHWYQQLPGTAPKLLIYGN | (Human) | (Human) | (Human) | (Human) | | GSNWYFDL | | DSV | (https://www.nature.co |
| | SNRPSGVPDRFSGSKSGTSASLAITGL | | | | | | | | | m/articles/s41591-020- |
| | QAEDEADYYCQSYDSSLSDSVFGGGT | | | | | | | | | 0998-x) |
| | KVTVL | | | | | | | | | |
| CoV2- | DIQLTQSPSFLSASVGDRVTITCRASQG | IGHV3-66 | IGHJ6 | IGKV1-9 | IGKJ5 | 2374 | ARDLVTYGLD | 3576 | QLLNSHPL | Seth Zost et al., 2020 |
| 2080 | ISNYLAWYQQKPGTAPNLLIYAASTLQ | (Human) | (Human) | (Human) | (Human) | | V | | T | (https://www.nature.co |
| | SGVPSRFSGSGSGTEFTLTISSLQPEDF | | | | | | | | | m/articles/s41591-020- |
| | ATYYCQLLNSHPLTFGQGTRLEIK | | | | | | | | | 0998-x) |
| CoV2- | SSELTQDPAVSVALGQTVRITCQGDSL | IGHV3-20 | IGHJ3 | IGLV3-19 | IGLJ3 | 2375 | AVIMSPIPRY | 3577 | NSRDSSGN | Seth Zost et al., 2020 |
| 2082 | RSYYASWYQQKPGQVPILVIYDKNNR | (Human) | (Human) | (Human) | (Human) | | SGYDWAGGAF | | AVV | (https://www.nature.co |
| | PSGIPDRFSGSSSGNTASLTITGAQAED | | | | | | DI | | | m/articles/s41591-020- |
| | EADYYCNSRDSSGNAVVFGGGTKLTV | | | | | | | | | 0998-x) |
| | L | | | | | | | | | |
| CoV2- | DIQMTQSPSSLSASVGDRVTITCQASQ | IGHV3-30 | IGHJ4 | IGKV1-33 | IGKJ3 | 2376 | AKNLGPYCSG | 3578 | QQYANLPF | Seth Zost et al., 2020 |
| 2083 | DISNYLNWYQQKPGKAPKLLIYDASNL | (Human) | (Human) | (Human) | (Human) | | GTCYSLVGDY | | T | (https://www.nature.co |
| | ETGVPSRFSGSGSGTDFTFTISSLQPED | | | | | | | | | m/articles/s41591-020- |
| | IATYYCQQYANLPFTFGPGTKVDIK | | | | | | | | | 0998-x) |
| CoV2- | SSELTQDPAVSVALGQTVRITCQGDSL | IGHV3-20 | IGHJ3 | IGLV3-19 | IGLJ2 | 2377 | AVIMSPIPR | 3579 | NSRDSSGN | Seth Zost et al., 2020 |
| 2094 | RSYYASWYQQKPGQVPILVIYDKNNR | (Human) | (Human) | (Human) | (Human) | | YSGYDWAGD | | AVV | (https://www.nature.co |
| | PSGIPDRFSGSSSGNTASLTITGAQAED | | | | | | AFDI | | | m/articles/s41591-020- |
| | EADYYCNSRDSSGNAVVFGGGTKLTV | | | | | | | | | 0998-x) |
| | L | | | | | | | | | |
| CoV2- | QSVLTQAPSASGTPGQRVTISCSGSNS | IGHV1-8 | IGHJ4 | IGLV1-44 | IGLJ2 | 2378 | ARMRSGWPT | 3580 | AVWDDSL | Seth Zost et al., 2020 |
| 2096 | NIGSYTINWYQQLPGTAPKLLIYGNDQ | (Human) | (Human) | (Human) | (Human) | | HGRPDDF | | NGLV | (https://www.nature.co |
| | RTSGVPDRFSGSKFGTSASLAISGLQSE | | | | | | | | | m/articles/s41591-020- |
| | DENNYYCAVWDDSLNGLVFGGGTKL | | | | | | | | | 0998-x) |
| | TVL | | | | | | | | | |
| CoV2- | DIQMTQSPSSLSASVGDRVTITCRASQ | IGHV3-9 | IGHJ6 | IGKV1-39 | IGKJ1 | 2379 | AKDIIRQGED | 3581 | QQSYSTP | Seth Zost et al., 2020 |
| 2097 | NIASYLNWYQQKPGKAPKLLIYAASSL | (Human) | (Human) | (Human) | (Human) | | GMDV | | WT | (https://www.nature.co |
| | QSGVPSRFSGSGSGTDFTLTISSLQPEE | | | | | | | | | m/articles/s41591-020- |
| | FATYYCQQSYSTPWTFGQGTKVEIK | | | | | | | | | 0998-x) |
| CoV2- | DIVMTQSPATLSVSPGERAILSCRASQ | IGHV3-23 | IGHJ3 | IGKV3-15 | IGKJ1 | 2380 | VKGLFDWFPL | 3582 | HQYNNWP | Seth Zost et al., 2020 |
| 2098 | SVRSNLAWYQQKPGQAPRLLISGAST | (Human) | (Human) | (Human) | (Human) | | | | QT | (https://www.nature.co |
| | RATAIPARFSGSGSGTEFTLTITSLQSED | | | | | | | | | m/articles/s41591-020- |
| | CAVYYCHQYNNWPQTFGQGTKVEIK | | | | | | | | | 0998-x) |
| CoV2- | NFMLTQPHSVSESPGKTVTISCTGSSG | IGHV3-7 | IGHJ4 | IGLV6-57 | IGLJ3 | 2381 | ARLGFYYGGA | 3583 | QSYDGINR | Seth Zost et al., 2020 |
| 2103 | SIASNYVQWYQQRPGSAPTTVISEDN | (Human) | (Human) | (Human) | (Human) | | DY | | AWV | (https://www.nature.co |
| | QRPSGVPDRFSGSIDSSSNSASLTISGL | | | | | | | | | m/articles/s41591-020- |
| | KTEDEADYYCQSYDGINRAWVFGGGT | | | | | | | | | 0998-x) |
| | KLTVL | | | | | | | | | |
| CoV2- | DIVMTQSPLSLPVTPGEPASISCRSSQS | IGHV3-9 | IGHJ4 | IGKV2-28 | IGKJ5 | 2382 | AKGVDYGGK | 3584 | MQALQTP | Seth Zost et al., 2020 |
| 2108 | LLHSLGYNSLSWYLQKPGQSPHLLIYL | (Human) | (Human) | (Human) | (Human) | | LAYFDS | | LT | (https://www.nature.co |
| | GSNRASGVPDRFSGSGSATDFTLKISR | | | | | | | | | m/articles/s41591-020- |
| | LEAEDVGVYYCMQALQTPLTFGQGTR | | | | | | | | | 0998-x) |
| | LEIK | | | | | | | | | |
| CoV2- | DIQMTQSPSSLSACVGDRVTITCRASQ | IGHV3-30- | IGHJ4 | IGKV1-39 | IGKJ5 | 2383 | ARDIDSGYDP | 3585 | QQSYSSL | Seth Zost et al., 2020 |
| 2110 | SISSYLNWYQQKPGKGPKLLIYAASSLQ | 3 (Human) | (Human) | (Human) | (Human) | | TPVFDY | | SIT | (https://www.nature.co |
| | SGVPSRFSGSGSGTDFTLTISSLQPEDF | | | | | | | | | m/articles/s41591-020- |
| | ATYYCQQSYSSLSITFGQGTRLEIK | | | | | | | | | 0998-x |
| CoV2- | DIQMTQSPSSLSASVGDRVTITCRASQ | IGHV3-13 | IGHJ5 | IGKV1-39 | IGKJ1 | 2384 | ARVLYDSSGF | 3586 | QQSYEIPP | Seth Zost et al., 2020 |
| 2111 | SISSYLNWYQQKPGKAPKLLIYAASSLE | (Human) | (Human) | (Human) | (Human) | | YNWFDP | | WT | (https://www.nature.co |
| | SGVPSRFSGSGSGTDFTLTISSLQPEDF | | | | | | | | | m/articles/s41591-020- |
| | ATYYCQQSYEIPPWTFGQGTKVEIK | | | | | | | | | 0998-x |
| CoV2- | DIQMTQSPSSLSASVGDRVTITCQASQ | IGHV3-53 | IGHJ5 | IGKV1-33 | IGKJ4 | 2385 | ARDFLRWHD | 3587 | QQYDNLP | Seth Zost et al., 2020 |
| 2113 | DINNYLNWYQQKPGKAPKLLIYDASN | (Human) | (Human) | (Human) | (Human) | | L | | PV | (https://www.nature.co |
| | LETGVPLRFSGSGSGTDFIFTISSLQPED | | | | | | | | | m/articles/s41591-020- |
| | IATYYCQQYDNLPPVFGGGTKVEIK | | | | | | | | | 0998-x) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV2 | SARS- | | SARS-CoV1, | S; RBD | B-cells; SARS-CoV2 | 298 | QVQLVQSGAEVKKPGSSVKVSCKASGD | 1377 |
| 2114 | | | CoV1 | | SARS-CoV2 | | Human Patient | | TFSSYTINWVRQAPGQGLEWMGRIIPIL | |
| | | | | | | | | | GIPNYAQKFQGRVTITADKSTSTAFMEL | |
| | | | | | | | | | SSLRSEDTAVYYCARGRGYSNYGASYYM | |
| | | | | | | | | | DVWGKGTTVTVSS | |
| CoV2- | Ab | SARS-CoV1, | | | SARS-CoV2 | S; RBD | B-cells; SARS-CoV2 | 299 | QLQLQESGPGLVKPSETLSLTCTVSGGSI | 1378 |
| 2128 | | SARS-CoV2 | | | | | Human Patient | | SSSSYYWGWIRQPPGNGLEWIGSIYYSG | |
| | | | | | | | | | STYYNPSLKGRVSISVDTSKNQFSLKLSSV | |
| | | | | | | | | | TAADTAVYYCARILVIFTLNWFDPWGQ | |
| | | | | | | | | | GTLVTVSS | |
| CoV2- | Ab | SARS-CoV2 | SARS- | SARS-CoV2 | | S; RBD | B-cells; SARS-CoV2 | 300 | EVQLVESGGGLVKPGGSLRLSCAASGFT | 1379 |
| 2130 | | | CoV1 | | | | Human Patient | | FRDVWMSWVRQAPGKGLEWVGRIKS | |
| | | | | | | | | | KIDGGTTDYAAPVKGRFTISRDDSKNTLY | |
| | | | | | | | | | LQMNSLKTEDTAVYYCTTAGSYYYDTVG | |
| | | | | | | | | | PGLPEGKFDYWGQGTLVTVSS | |
| CoV2- | Ab | SARS-CoV2 | SARS- | SARS-CoV2 | | S; RBD | B-cells; SARS-CoV2 | 301 | EVQLVESGGGLIQPGGSLRLSCAASEVT | 1380 |
| 2132 | | | CoV1 | | | | Human Patient | | VSSNYMSWVRQAPGKGLEWVSLIYSGG | |
| | | | | | | | | | TTYYADSVKGRFTISRDNSKNTLYLQMN | |
| | | | | | | | | | SLRAEDTAVYYCARDFLRWHDLWGQG | |
| | | | | | | | | | TLVTVSS | |
| CoV2- | Ab | SARS-CoV2 | SARS- | | SARS-CoV1, | S; RBD | B-cells; SARS-CoV2 | 302 | QVQLQESGPGLVKPSETLSLTCTVSGGS | 1381 |
| 2137 | | | CoV1 | | SARS-CoV2 | | Human Patient | | VSSGSYYWSWIRQPPGKGLECIGYIYYSG | |
| | | | | | | | | | SSNYNPSLKSRVTISVDTSKNQFSLKMSS | |
| | | | | | | | | | VTAADTAVYYCAGSPVPPTIVGASYWG | |
| | | | | | | | | | QGTLVTVSS | |
| CoV2- | Ab | SARS-CoV2 | SARS- | | SARS-CoV1, | S; non- | B-cells; SARS-CoV2 | 303 | EVQLVQSGAEVKKPGESLKISCKGSGYSF | 1382 |
| 2142 | | | CoV1 | | SARS-CoV2 | RBD | Human Patient | | TSYWIDWVRQMPGKGLEWMGIIYPGD | |
| | | | | | | | | | SDTRYSPSFQGQVTISADKSTSTAYLQW | |
| | | | | | | | | | SSLKASDTAMYYCARRGEAAGIWYFDL | |
| | | | | | | | | | WGRGTLVTVSS | |
| CoV2- | Ab | SARS-CoV2 | SARS- | | SARS-CoV1, | S; non- | B-cells; SARS-CoV2 | 304 | EVQLVESGGGLVQPGGSLRLSCAASGFT | 1383 |
| 2143 | | | CoV1 | | SARS-CoV2 | RBD | Human Patient | | VSSNYMSWVRQAPGKGLEWVSVIYSA | |
| | | | | | | | | | GSTYYADSVKGRFSISRDKSKNTLYLQM | |
| | | | | | | | | | NSLRAEDTAVYYCAKEGGSGSLRYYYYG | |
| | | | | | | | | | MDVWGQGTTVTVSS | |
| CoV2- | Ab | SARS-CoV2 | SARS- | | SARS-CoV1, | S; NTD | B-cells; SARS-CoV2 | 305 | EVQLVESGGGLVQPGGSLRLSCEASGFT | 1384 |
| 2146 | | | CoV1 | | SARS-CoV2 | | Human Patient | | FSSSEINWVRQAPGKGLEWVSHISSSGSI | |
| | | | | | | | | | IYYADSVKGRFTISRDNAKNSLYLQMNSL | |
| | | | | | | | | | RAEDTAVYYCARRSYRSSWYYYYGMDV | |
| | | | | | | | | | WGQGTTVTVSS | |
| CoV2- | Ab | SARS-CoV2 | SARS- | | SARS-CoV1, | S; non- | B-cells; SARS-CoV2 | 306 | QVQLAESGGGVVQPGRSLRLSCAASGF | 1385 |
| 2147 | | | CoV1 | | SARS-CoV2 | RBD | Human Patient | | TFSSYAMHWVRQAPGKGLEWVAVISY | |
| | | | | | | | | | DGSNKYYADSVKGRFTISRDNSKNTLYL | |
| | | | | | | | | | QMNSLRAEDTAVYYCARSTSGSYYYGM | |
| | | | | | | | | | DVWGQGTTVTVSS | |
| CoV2- | Ab | SARS-CoV2 | SARS- | | SARS-CoV1, | S; NTD | B-cells; SARS-CoV2 | 307 | QVQLVESGGGVVQPGRSLRLSCAASGF | 1386 |
| 2150 | | | CoV1 | | SARS-CoV2 | | Human Patient | | TFSTYAMHWVRQAPGKGLEWVAVISY | |
| | | | | | | | | | DGSNKYYADSVKGRFTISRDNSKNTLYL | |
| | | | | | | | | | QMIGLRAEDTAVYYCARDWAPTYYDM | |
| | | | | | | | | | PSAFDIWGQGTMVTVSS | |
| CoV2- | Ab | SARS-CoV1, | | | SARS-CoV2 | S; non- | B-cells; SARS-CoV2 | 308 | QVQLVQSGAEVRKPGSSVKVSCKASGG | 1387 |
| 2151 | | SARS-CoV2 | | | | RBD | Human Patient | | TFSSYAISWVRQAPGQGLEWMGGIIPV | |
| | | | | | | | | | FGTANYAQKFQGRVTITADKSTSTAFME | |
| | | | | | | | | | LNSLRSEDTAVYYCARIGSYPEYFQHWG | |
| | | | | | | | | | QGTLVTVSS | |
| CoV2- | Ab | SARS-CoV1, | | | SARS-CoV2 | S; RBD | B-cells; SARS-CoV2 | 309 | QVQLQESGPGLVKPSQTLSLTCTVSGGSI | 1388 |
| 2158 | | SARS-CoV2 | | | | | Human Patient | | SSGGYFWSWIRQHPGKGLEWIGSIYYS | |
| | | | | | | | | | GSTYYNPSLRSRITISVDTSKNQFSLKLSS | |
| | | | | | | | | | VTAADTAVYYCARGGSGSYSLFDYWGQ | |
| | | | | | | | | | GTLVTVSS | |
| CoV2- | Ab | SARS-CoV1, | | | SARS-CoV2 | S; non- | B-cells; SARS-CoV2 | 310 | QVQLAESGGGVVQPGRSLRLSCAASGF | 1389 |
| 2159 | | SARS-CoV2 | | | | RBD | Human Patient | | TFSSYAMHWVRQAPGKGLEWVAVISY | |
| | | | | | | | | | DGSNKYYADSVKGRFTISRDNSKNTLYL | |
| | | | | | | | | | QMNSLRAEDTAVYYCARSTSGSYYYGM | |
| | | | | | | | | | DVWGQGTTVTVSS | |
| CoV2- | Ab | SARS-CoV1, | | | SARS-CoV2 | S; non- | B-cells; SARS-CoV2 | 311 | QVQLAESGGGVVQPGRSLRLSCAASGF | 1390 |
| 2160 | | SARS-CoV2 | | | | RBD | Human Patient | | TFSSYAMHWVRQAPGKGLEWVAVISY | |
| | | | | | | | | | DGSNKYYADSVKGRFTISRDNSKNTLYL | |
| | | | | | | | | | QMNSLRAEDTAVYYCARSTSGSYYYGM | |
| | | | | | | | | | DVWGQGTTVTVSS | |
| CoV2- | Ab | SARS-CoV2 | SARS- | SARS-CoV2 | | S; RBD | B-cells; SARS-CoV2 | 312 | EVQLVESGGGLVQPGGSLRLSCAASGLT | 1391 |
| 2165 | | | CoV1 | | | | Human Patient | | VRSNYMTWVRQTPGKGLEWVSVIYSG | |
| | | | | | | | | | GSTFYADSVKGRFTISRDNSKNTVYLQM | |
| | | | | | | | | | NSLRAEDTAVYYCARDLVTYGLDVWGQ | |
| | | | | | | | | | GTTVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | QSVLTQPPSVSGAPGQRVTISCTGSSS | IGHV1-69 | IGHJ6 | IGLV1-40 | IGLJ2 | 2386 | ARGRGYSNY | 3588 | QSYDSSLS | Seth Zost et al., 2020 |
| 2114 | NIGAGYDVHWYQQLPETAPKLLIYAN | (Human) | (Human) | (Human) | (Human) | | GASYYMDV | | GSV | (https://www.nature.co |
| | SNRPSGVPDRFSGSKSGTSASLAITGL | | | | | | | | | m/articles/s41591-020- |
| | QAEDEADYYCQSYDSSLSGSVFGGGT | | | | | | | | | 0998-x) |
| | KLTVL | | | | | | | | | |
| CoV2- | NFMLTQPHSVSESPGKTVTISCTGSSG | IGHV4-39 | IGHJ5 | IGLV6-57 | IGLJ2 | 2387 | ARILVIFTL | 3589 | QSYDSGNP | Seth Zost et al., 2020 |
| 2128 | SIASNYVQWYQQRPGSAPTTVIYEDN | (Human) | (Human) | (Human) | (Human) | | NWFDF | | | (https://www.nature.co |
| | QRPSGVPDRFSGSIDSSSNSASLTISGL | | | | | | | | | m/articles/s41591-020- |
| | KTEDEADYYCQSYDSGNPIFGGGTKLT | | | | | | | | | 0998-x |
| | VL | | | | | | | | | |
| CoV2- | DIVMTQSPDSLAVSLGERATINCKSSQ | IGHV3-15 | IGHJ4 | IGKV4-1 | IGKJ4 | 2388 | TTAGSYYYDT | 3590 | QQYYSTLT | Seth Zost et al., 2020 |
| 2130 | SVLYSSNNKNYLAWYQQKPGQPPKLL | (Human) | (Human) | (Human) | (Human) | | VGPGLPEGKF | | | (https://www.nature.co |
| | MYWASTRESGVPDRFSGSGSGAEFTL | | | | | | DY | | | m/articles/s41591-020- |
| | TISSLQAEDVAIYYCQQYYSTLTFGGGT | | | | | | | | | 0998-x) |
| | KVEIK | | | | | | | | | |
| CoV2- | DIQMTQSPSSLSASVGDRVTITCQASQ | IGHV3-53 | IGHJ5 | IGKV1-33 | IGKJ4 | 2389 | ARDFLRWHD | 3591 | QQYDNLP | Seth Zost et al., 2020 |
| 2132 | DINNYLNWYQQKPGKAPKLLIYDASN | (Human) | (Human) | (Human) | (Human) | | L | | PV | (https://www.nature.co |
| | LETGVPLRFSGSGSGTDFTFTISSLQPE | | | | | | | | | m/articles/s41591-020- |
| | DIATYYCQQYDNLPPVFGGGTKVEIK | | | | | | | | | 0998-x) |
| CoV2- | NFMLTQPHSVSESPGKTVTFSCTGSSG | IGHV4-61 | IGHJ4 | IGLV6-57 | IGLJ2 | 2390 | AGSPVPPTIV | 3592 | QSYDGINR | Seth Zost et al., 2020 |
| 2137 | SIASNYVQWYQQRPGSAPTTVIYEDN | (Human) | (Human) | (Human) | (Human) | | GASY | | WLV | (https://www.nature.co |
| | QRPSGVPDRFSGSIDSSSNSASLTISGL | | | | | | | | | m/articles/s41591-020- |
| | KTEDEADYYCQSYDGINRWLVFGGGT | | | | | | | | | 0998-x) |
| | KLTVL | | | | | | | | | |
| CoV2- | QPVLTQPPSASASLGASVTLTCTLSSGY | IGHV5-51 | IGHJ2 | IGLV9-49 | IGLJ2 | 2391 | ARRGEAAGI | 3593 | GADHGSG | Seth Zost et al., 2020 |
| 2142 | SNYKVDWYQQRPGKGPRFVMRVGT | (Human) | (Human) | (Human) | (Human) | | WYFDL | | SNFEYVV | (https://www.nature.co |
| | GGIVGSKGDGIPDRFSVLGSGLNRYLTI | | | | | | | | | m/articles/s41591-020- |
| | KNIQEEDESDYHCGADHGSGSNFEYV | | | | | | | | | 0998-x) |
| | VFGGGTKLTVL | | | | | | | | | |
| CoV2- | QSVVTQPPSASGTPGQRVTISCSGSSS | IGHV3-66 | IGHJ6 | IGLV1-44 | IGLJ1 | 2392 | AKEGGSGSLR | 3594 | AAWDDSL | Seth Zost et al., 2020 |
| 2143 | NIGYNIVNWYQQLPGTAPKLLIYSNN | (Human) | (Human) | (Human) | (Human) | | YYYYGMDV | | NGYV | (https://www.nature.co |
| | QRPSGVPDRFSGSKSGTSASLSISGLQS | | | | | | | | | m/articles/s41591-020- |
| | EDEADYYCAAWDDSLNGYVFGTGTK | | | | | | | | | 0998-x) |
| | VTVL | | | | | | | | | |
| CoV2- | DIQLTQSPSFLSASVGDRVTITCRASQG | IGHV3-48 | IGHJ6 | IGKV1-9 | IGKJ1 | 2393 | ARRSYRSSWY | 3595 | QQLNSYPV | Seth Zost et al., 2020 |
| 2146 | ISSYLAWYQQKPGKAPKLLIYAASTLQS | (Human) | (Human) | (Human) | (Human) | | YYYGMDV | | T | (https://www.nature.co |
| | GVPSRFSGSGSGTEFTLTISSLQPEDFA | | | | | | | | | m/articles/s41591-020- |
| | TYYCQQLNSYPVTFGQGTKVEIK | | | | | | | | | 0998-x) |
| CoV2- | QSALTQPASVSGSPGQSITISCTGTSSD | IGHV3-30- | IGHJ6 | IGLV2-14 | IGLJ1 | 2394 | ARSTSGSYYY | 3596 | SSYTSSST | Seth Zost et al., 2020 |
| 2147 | VGDYNYVSWYQQHPGKAPKLMIYDV | 3 | (Human) | (Human) | (Human) | | GMDV | | LYV | (https://www.nature.co |
| | SNRPSGVSNRFSGSKSGNTASLTISGL | (Human) | | | | | | | | m/articles/s41591-020- |
| | QAEDEAEYYCSSYTSSSTLLYVFGTGTK | | | | | | | | | 0998-x) |
| | VTVL | | | | | | | | | |
| CoV2- | SYVLTQPPSVSVAPGKTARITCGGNNI | IGHV3-30- | IGHJ3 | IGLV3-21 | IGLJ3 | 2395 | ARDWAPTYY | 3597 | QVWDSSS | Seth Zost et al., 2020 |
| 2150 | GNKGVHWYQQKPGQAPVLVVDDDS | 3 | (Human) | (Human) | (Human) | | DMPSAFDI | | DHPGV | (https://www.nature.co |
| | DRPSGIPERFSGSNSGNTATLIISSVEV | (Human) | | | | | | | | m/articles/s41591-020- |
| | GDEADFYCQVWDSSSDHPGVFGGGT | | | | | | | | | 0998-x) |
| | KLTVL | | | | | | | | | |
| CoV2- | EIVLTQSPATLSLSPGERATLSCRASQS | IGHV1-69 | IGHJ1 | IGKV3-11 | IGKJ4 | 2396 | ARIGSYPEY | 3598 | HYRSNWP | Seth Zost et al., 2020 |
| 2151 | VSSFLAWYQQKPGQAPRLLIYDASNR | (Human) | (Human) | (Human) | (Human) | | FQH | | PVLT | (https://www.nature.co |
| | ATGIPARFSGSGSGTDFTLTISSLEPEDF | | | | | | | | | m/articles/s41591-020- |
| | AVYYCHYRSNWPPVLTFGGGTKVEIE | | | | | | | | | 0998-x) |
| CoV2- | DIQMTQSPSSLSASVGDRVTITCQASQ | IGHV4-31 | IGHJ4 | IGKV1-33 | IGKJ2 | 2397 | ARGGSGSYSL | 3599 | QQYDNLYS | Seth Zost et al., 2020 |
| 2158 | DITNYLNWYQQKPGKAPKLLIYDASNL | (Human) | (Human) | (Human) | (Human) | | FDY | | VH | (https://www.nature.co |
| | ETGVPSRFSGSGSGTDFTFTISSLQPED | | | | | | | | | m/articles/s41591-020- |
| | FATYYCQQYDNLYSVHFGQGTKLEIK | | | | | | | | | 0998-x) |
| CoV2- | QSALTQPASVSGSPGQSITISCTGTSSD | IGHV3-30- | IGHJ6 | IGLV2-14 | IGLJ1 | 2398 | ARSTSGSYYY | 3600 | SSYTSSST | Seth Zost et al., 2020 |
| 2159 | VGDYNYVSWYQQHPGKAPKLMIYDV | 3 | (Human) | (Human) | (Human) | | GMDV | | LYV | (https://www.nature.co |
| | SNRPSGVSNRFSGSKSGNTASLTISGL | (Human) | | | | | | | | m/articles/s41591-020- |
| | QAEDEAEYYCSSYTSSSTLLYVFGTGTK | | | | | | | | | 0998-x) |
| | VTVL | | | | | | | | | |
| CoV2- | QSALTQPASVSGSPGQSITISCTGTSSD | IGHV3-30- | IGHJ6 | IGLV2-14 | IGLJ1 | 2399 | ARSTSGSYYY | 3601 | SSYTSSST | Seth Zost et al., 2020 |
| 2160 | VGDYNYVSWYQQHPGKAPKLMIYDV | 3 | (Human) | (Human) | (Human) | | GMDV | | LYV | (https://www.nature.co |
| | SNRPSGVSNRFSGSKSGNTASLTISGL | (Human) | | | | | | | | m/articles/s41591-020- |
| | QAEDEAEYYCSSYTSSSTLLYVFGTGTK | | | | | | | | | 0998-x) |
| | VTVL | | | | | | | | | |
| CoV2- | DIQLTQSPSFLSASVGDRVTITCRASQG | IGHV3-66 | IGHJ6 | IGKV1-9 | IGKJ5 | 2400 | ARDLVTYGLD | 3602 | QLLNSHPL | Seth Zost et al., 2020 |
| 2165 | ISNYLAWYQQKPGTAPNLLIYAASTLQ | (Human) | (Human) | (Human) | (Human) | | V | | T | (https://www.nature.co |
| | SGVPSRFSGSGSGTEFTLTISSLQPEDF | | | | | | | | | m/articles/s41591-020- |
| | ATYYCQLLNSHPLTFGQGTRLEIK | | | | | | | | | 0998-x) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV1, | | | SARS-CoV2 | S; non- | B-cells; SARS-CoV2 | 313 | QVQLVQSGAEVKKPGSSVKVSCKASGG | 1392 |
| 2166 | | SARS-CoV2 | | | | RBD | Human Patient | | TFSSYAIIWVRQAPGQGLEWMGGIIPIF | |
| | | | | | | | | | GTTNYAQKFQGRVTITADESTSTAYVELS | |
| | | | | | | | | | SLRSEDTAVYYCARIGHFDSSGYYLDYW | |
| | | | | | | | | | GQGTLVTVSS | |
| CoV2- | Ab | SARS-CoV1, | | | SARS-CoV2 | S; non- | B-cells; SARS-CoV2 | 314 | EVQLVESGGGLVQPGGSLRLSCVASGFT | 1393 |
| 2171 | | SARS-CoV2 | | | | RBD | Human Patient | | FSFYWMSWVRQAPGKGLEWVANIKQ | |
| | | | | | | | | | DGGEKYYVDSVKGRFTISRDNAKNSLYL | |
| | | | | | | | | | QMNSLRAEDTAVYYCARLSGSSWDFDY | |
| | | | | | | | | | WGQGTLVTVSS | |
| CoV2- | Ab | SARS-CoV2 | SARS- | | SARS-CoV1, | S; non- | B-cells; SARS-CoV2 | 315 | EVQLVESGGGVVRPGGSLRLSCAASGFT | 1394 |
| 2173 | | | CoV1 | | SARS-CoV2 | RBD | Human Patient | | FDDYGMSWVRQAPGKGLEWVSAINW | |
| | | | | | | | | | NGGSTGYADSVKGRFTISRDNAKNSLYL | |
| | | | | | | | | | QMNSLRAEDTALYHCARRRSSSRYSSG | |
| | | | | | | | | | WYMYYYYMDVWGKGTTVTVSS | |
| CoV2- | Ab | SARS-CoV1, | | | SARS-CoV2 | S; non- | B-cells; SARS-CoV2 | 316 | EVQLVESGGGLVQPGGSLRLSCVASGFT | 1395 |
| 2175 | | SARS-CoV2 | | | | RBD | Human Patient | | FSFYWMSWVRQAPGKGLEWVANIKQ | |
| | | | | | | | | | DGGEKYYVDSVKGRFTISRDNAKNSLYL | |
| | | | | | | | | | QMNSLRAEDTAVYYCARLSGSSWDFDY | |
| | | | | | | | | | WGQGTLVTVSS | |
| CoV2- | Ab | SARS-CoV2 | SARS- | | SARS-CoV1, | S; non- | B-cells; SARS-CoV2 | 317 | QITFKESGPTLVKPTETLTLTCTFSGFSVS | 1396 |
| 2177 | | | CoV1 | | SARS-CoV2 | RBD | Human Patient | | TSGEGVGWIRQPPGKALEWLAVIYWD | |
| | | | | | | | | | DDKRYSPSLKSRLTITRDTSKNQVVLTMT | |
| | | | | | | | | | NMDPVDTATYYCAHRLWFRDAFDIWG | |
| | | | | | | | | | QGTTVTVSS | |
| CoV2- | Ab | SARS-CoV1, | | | SARS-CoV2 | S; non- | B-cells; SARS-CoV2 | 318 | EVQLVESGGGLVQPGGSLRLSCAASGFT | 1397 |
| 2178 | | SARS-CoV2 | | | | RBD | Human Patient | | FSTYWMTWVRQAPGKGLEWVANIKQ | |
| | | | | | | | | | DGSEKYYVDSVKYRFTISRDNAKNSLYLQ | |
| | | | | | | | | | MNSLRAEDTAVYYCARVGSSSWYFDYW | |
| | | | | | | | | | GQGTLVTVSS | |
| CoV2- | Ab | SARS-CoV1, | | | SARS-CoV2 | S; non- | B-cells; SARS-CoV2 | 319 | QVQLVESGGGVVQPGRSLRLSCAASGF | 1398 |
| 2183 | | SARS-CoV2 | | | | RBD | Human Patient | | TFSSYAMHWVRQAPGKGLEWVAGISY | |
| | | | | | | | | | DGSNKYYADSVKGRFTISRDNSKNTLYL | |
| | | | | | | | | | QMNSLRAEDTAVYYCARADTMVRGTYF | |
| | | | | | | | | | EYWGQGTLVTVSS | |
| CoV2- | Ab | SARS-CoV1, | | | SARS-CoV2 | S; non- | B-cells; SARS-CoV2 | 320 | QVQLVESGGGVVQPGRSLRLSCAASGF | 1399 |
| 2187 | | SARS-CoV2 | | | | RBD | Human Patient | | TFSYYPMHWLWVRQAPGKGLEWVAVT | |
| | | | | | | | | | SYDGTNKYYADSVKGRFTISRDNSKNTLY | |
| | | | | | | | | | LQMNSLRAEDTAVYYCARGGATNFDY | |
| | | | | | | | | | WGQGTLVTVSS | |
| CoV2- | Ab | SARS-CoV1, | | | SARS-CoV2 | S; non- | B-cells; SARS-CoV2 | 321 | EAQLVESGGGLVQPGRSLRLSCAASGFT | 1400 |
| 2189 | | SARS-CoV2 | | | | RBD | Human Patient | | FDDSAMHWVRQAPGKGLEWVSGISW | |
| | | | | | | | | | NSGNVGYADSVKGRFTISRDNAKNSLYL | |
| | | | | | | | | | QMNSLRAEDTALYYCTKASRYCSSTICY | |
| | | | | | | | | | WNWFDPWGQGTLVTVSS | |
| CoV2- | Ab | SARS-CoV2 | SARS- | | SARS-CoV1, | S; NTD | B-cells; SARS-CoV2 | 322 | EVQLVESGGGLVQPGGSLRLSCAASGFT | 1401 |
| 2190 | | | CoV1 | | SARS-CoV2 | | Human Patient | | FSSYEMNWVRQAPGKGLEWVSYISSSG | |
| | | | | | | | | | SAIYYADSVKGRFTISRDNAKNSLYLQM | |
| | | | | | | | | | NSLRVEDTAVYYCAREARSRYFDWLPSY | |
| | | | | | | | | | YFDYWGQGTLVTVSS | |
| CoV2- | Ab | SARS-CoV2 | SARS- | | SARS-CoV1, | S; non- | B-cells; SARS-CoV2 | 323 | EVQLVESGGGVVRPGGSLRLSCAASGFT | 1402 |
| 2191 | | | CoV1 | | SARS-CoV2 | RBD | Human Patient | | FDDYGMSWVRQAPGKGLEWVSAINW | |
| | | | | | | | | | NGGSTGYADSVKGRFTISRDNAKNSLYL | |
| | | | | | | | | | QMNSLRAEDTALYHCARRRSSSRYSSG | |
| | | | | | | | | | WYMYYYYMDVWGKGTTVTVSS | |
| CoV2- | Ab | SARS-CoV2 | SARS- | | SARS-CoV1, | S; non- | B-cells; SARS-CoV2 | 324 | QVHLVESGGGVVQPGRSLRLSCAASGF | 1403 |
| 2195 | | | CoV1 | | SARS-CoV2 | RBD | Human Patient | | TFSNYGMHWVRQAPGKGLEWVAVISN | |
| | | | | | | | | | DEFNKFYANSVKGRFTISRDNSKNTVYL | |
| | | | | | | | | | QLNSLRTEDTARYYCAKGGDGSGWAW | |
| | | | | | | | | | DGDNPPTDYWGQGTLVIVSS | |
| CoV2- | Ab | SARS-CoV2 | SARS- | SARS-CoV2 | | S; RBD | B-cells; SARS-CoV2 | 325 | QMQLVQSGPEVKKPGTSVKVSCKASGF | 1404 |
| 2196 | | | CoV1 | | | | Human Patient | | TFMSSAVQWVRQARGQRLEWIGWIVI | |
| | | | | | | | | | GSGNTNYAQKFQERVTITRDMSTSTAY | |
| | | | | | | | | | MELSSLRSEDTAVYYCAAPYCSSISCNDG | |
| | | | | | | | | | FDIWGQGTMVTVSS | |
| CoV2- | Ab | SARS-CoV2 | SARS- | | SARS-CoV1, | S; non- | B-cells; SARS-CoV2 | 326 | EVQLVQSGAEVKKPGESLKISCKGSGYSF | 1405 |
| 2197 | | | CoV1 | | SARS-CoV2 | RBD | Human Patient | | TSYWIGWVRQMPGKGLEWMGIIYPGD | |
| | | | | | | | | | SDTRYSPSFQGQVTISADKSISTAYLQWS | |
| | | | | | | | | | SLKASDTAMYYCARPDYSSGWFSYWYF | |
| | | | | | | | | | DLWGRGTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | EIVLTQSPATLSLSPGERATLSCRASQS | IGHV1-69 | IGHJ4 | IGKV3-11 | IGKJ3 | 2401 | ARIGHFDSSG | 3603 | QHRTNWP | Seth Zost et al., 2020 |
| 2166 | VSSFLAWYQQKPGQAPRLLIYDASNR | (Human) | (Human) | (Human) | (Human) | | YYLDY | | PLFT | (https://www.nature.co |
| | PTGIPARFTGSGSGTDFTLTISSLEPEDF | | | | | | | | | m/articles/s41591-020- |
| | AVYYCQHRTNWPPLFTFGPGTKVDIK | | | | | | | | | 0998-x) |
| CoV2- | SYELTQPPSVSVSPGQTASITCSGDKLG | IGHV3-7 | IGHJ4 | IGLV3-1 | IGLJ1 | 2402 | ARLSGSSWDF | 3604 | QAWDSST | Seth Zost et al., 2020 |
| 2171 | DKYACWYQQRPGQSPVLVIYQDSKRP | (Human) | (Human) | (Human) | (Human) | | DY | | GV | (https://www.nature.co |
| | SGIPERFSGSNSGNTATLTISGTQAMD | | | | | | | | | m/articles/s41591-020- |
| | EADYYCQAWDSSTGVFGTGTKVTVL | | | | | | | | | 0998-x) |
| CoV2- | DIQMTQSPSTLSASVGDRVTITCRASQ | IGHV3-20 | IGHJ6 | IGKV1-5 | IGKJ1 | 2403 | ARRRSSSRYS | 3605 | QQYNTYSG | Seth Zost et al., 2020 |
| 2173 | SVSTWLAWYQQKPGKAPNLLIYEASSL | (Human) | (Human) | (Human) | (Human) | | SGWYMYYYY | | T | (https://www.nature.co |
| | ESGVPSRFSGSGSGTEFTLTISSLQPDD | | | | | | MDV | | | m/articles/s41591-020- |
| | FATYYCQQYNTYSGTFGQGTKVEIK | | | | | | | | | 0998-x) |
| CoV2- | SYELTQPPSVSVSPGQTASITCSGDKLG | IGHV3-7 | IGHJ4 | IGLV3-1 | IGLJ1 | 2404 | ARLSGSSWDF | 3606 | QAWDSST | Seth Zost et al., 2020 |
| 2175 | DKYACWYQQRPGQSPVLVIYQDSKRP | (Human) | (Human) | (Human) | (Human) | | DY | | GV | (https://www.nature.co |
| | SGIPERFSGSNSGNTATLTISGTQAMD | | | | | | | | | m/articles/s41591-020- |
| | EADYYCQAWDSSTGVFGTGTKVTVL | | | | | | | | | 0998-x) |
| CoV2- | DIQMTQSPSSLSASVGDRVTITCRARQ | IGHV2-5 | IGHJ3 | IGKV1-39 | IGKJ1 | 2405 | AHRLWFRDA | 3607 | QQTYSTF | Seth Zost et al., 2020 |
| 2177 | SISNYLNWYQQKPGKAPKLLIYAASSL | (Human) | (Human) | (Human) | (Human) | | FDI | | WT | (https://www.nature.co |
| | HSGVPSRFSGSGSGTDFTLTISSLQPED | | | | | | | | | m/articles/s41591-020- |
| | FATYYCQQTYSTFWTFGQGTNVEIK | | | | | | | | | 0998-x) |
| CoV2- | SYELTQPPSVSVSPGQTASITCSGDKLG | IGHV3-7 | IGHJ4 | IGLV3-1 | IGLJ2 | 2406 | ARVGSSSWYF | 3608 | QAWDSST | Seth Zost et al., 2020 |
| 2178 | DKYACWYQQKPGQSPVVVIYQDSKR | (Human) | (Human) | (Human) | (Human) | | DY | | AV | (https://www.nature.co |
| | PSGIPERFSGSNSGNTATLTISGTQAM | | | | | | | | | m/articles/s41591-020- |
| | DEADYYCQAWDSSTAVFGGGTKLTVL | | | | | | | | | 0998-x) |
| CoV2- | QSALTQPASVSGSPGQSITISCTGTSSD | IGHV3-30- | IGHJ4 | IGLV2-14 | IGLJ2 | 2407 | ARADTMVRG | 3609 | SSYTSSR | Seth Zost et al., 2020 |
| 2183 | VGGYNYVSWYQQHPGKAPKLMIYDV | 3 | (Human) | (Human) | (Human) | | TYFEY | | AVL | (https://www.nature.co |
| | SNRPSGVSNRFSGSKSGNTASLTISGL | (Human) | | | | | | | | m/articles/s41591-020- |
| | QAEDEADYCCSSYTSSRAVLFGGGTKL | | | | | | | | | 0998-x) |
| | TVL | | | | | | | | | |
| CoV2- | SYVLTQPPSVSVAPGKTANITCGGNNI | IGHV3-30- | IGHJ4 | IGLV3-21 | IGLJ3 | 2408 | ARGGATNFD | 3610 | QVWDSSS | Seth Zost et al., 2020 |
| 2187 | GRKSVHWYQQKSGQAPVLVVYDDSD | 3 | (Human) | (Human) | (Human) | | Y | | DHPEWV | (https://www.nature.co |
| | RPSGIPERFSGSNSGNTATLTISRVEAG | (Human) | | | | | | | | m/articles/s41591-020- |
| | DEADYYCQVWDSSSDHPEWVFGGGT | | | | | | | | | 0998-x) |
| | KLTVL | | | | | | | | | |
| CoV2- | DIQMTQSPSSLSASVGDRVTITCRASQ | IGHV3-9 | IGHJ5 | IGKV1-39 | IGKJ4 | 2409 | TKASRYCSST | 3611 | QQSYSTPT | Seth Zost et al., 2020 |
| 2189 | SISSYLNWYQQKPGKAPKLLIYGASSLQ | (Human) | (Human) | (Human) | (Human) | | ICYWNWFDP | | | (https://www.nature.co |
| | TGVPSRFSGSGSGTDFTLTIRSLQPEDF | | | | | | | | | m/articles/s41591-020- |
| | ASYYCQQSYSTPTFGGGTKVEIK | | | | | | | | | 0998-x) |
| CoV2- | QSALTQPASVSGSPGQSITISCTGTSSD | IGHV3-48 | IGHJ4 | IGLV2-14 | IGLJ2 | 2410 | AREARSRYFD | 3612 | SSYTSSST | Seth Zost et al., 2020 |
| 2190 | IGGYNYVSWYQQHPGKAPKLLIYDVS | (Human) | (Human) | (Human) | (Human) | | WLPSYYFDY | | HVV | (https://www.nature.co |
| | NRPSGVSTRFSGSKSGNTASLTISGLQ | | | | | | | | | m/articles/s41591-020- |
| | AEDEADYYCSSYTSSSTHVVFGGGTKL | | | | | | | | | 0998-x) |
| | TVL | | | | | | | | | |
| CoV2- | DIQMTQSPSTLSASVGDRVTITCRASQ | IGHV3-20 | IGHJ6 | IGKV1-5 | IGKJ1 | 2411 | ARRRSSSRYS | 3613 | QQYNTYSG | Seth Zost et al., 2020 |
| 2191 | SVSTWLAWYQQKPGKAPNLLIYEASSL | (Human) | (Human) | (Human) | (Human) | | SGWYMYYYY | | T | (https://www.nature.co |
| | ESGVPSRFSGSGSGTEFTLTISSLQPDD | | | | | | MDV | | | m/articles/s41591-020- |
| | FATYYCQQYNTYSGTFGQGTKVEIK | | | | | | | | | 0998-x) |
| CoV2- | DIVMTQSPDFLAVSLGERATISCKSSQS | IGHV3-30 | IGHJ4 | IGKV4-1 | IGKJ4 | 2412 | AKGGDGSG | 3614 | QQYYTAPL | Seth Zost et al., 2020 |
| 2195 | VLYTPKNKNYLAWYKQKPGQPPKVLIY | (Human) | (Human) | (Human) | (Human) | | WAWDGDNP | | T | (https://www.nature.co |
| | WASTRESGVPDRFSGSGSGTDFTLIISS | | | | | | PTDY | | | m/articles/s41591-020- |
| | LQAEDAAVYYCQQYYTAPLTFGGGTR | | | | | | | | | 0998-x) |
| | VEIK | | | | | | | | | |
| CoV2- | EIVLTQSPGTLSLSPGERATLSCRASQS | IGHV1-58 | IGHJ3 | IGKV3-20 | IGKJ1 | 2413 | AAPYCSSIS | 3615 | QHYGSSRG | Seth Zost et al., 2020 |
| 2196 | VSSSYLAWYQQKPGQAPRLLIYGASSR | (Human) | (Human) | (Human) | (Human) | | CNDGFDI | | WT | (https://www.nature.co |
| | ATGIPDRFSGSGSGTDFTLTISRLEPED | | | | | | | | | m/articles/s41591-020- |
| | FAVYYCQHYGSSRGWTFGQGTKVEIK | | | | | | | | | 0998-x) |
| CoV2- | EIVLTQSPGTLSLSPGERATLSCRASQS | IGHV5-51 | IGHJ2 | IGKV3-20 | IGKJ5 | 2414 | ARPDYSSGW | 3616 | QQYGRSPI | Seth Zost et al., 2020 |
| 2197 | VSSNFLAWYQQKPGQAPRLLIYGASS | (Human) | (Human) | (Human) | (Human) | | FSYWYFDL | | T | (https://www.nature.co |
| | RATGIPDRFSGSGSGTDFTLTISRLEPE | | | | | | | | | m/articles/s41591-020- |
| | DFAVYYCQQYGRSPITFGQGTRLEIK | | | | | | | | | 0998-x) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV2 | SARS- | | SARS-CoV1, | S; NTD | B-cells; SARS-CoV2 | 327 | QVQLVQSGAEVKKPGASVKVSCKVSGY | 1406 |
| 2199 | | | CoV1 | | SARS-CoV2 | | Human Patient | | TLTELSIHWVRQAPGKGLEWMGGFDP | |
| | | | | | | | | | EDAETIYAQQFQGRVTMTEDTSTDTAY | |
| | | | | | | | | | MELSSLKSEDTALYYCATGFAVFGRAAV | |
| | | | | | | | | | PYWGQGTLVTVSS | |
| CoV2- | Ab | SARS-CoV1, | | | SARS-CoV2 | S; non- | B-cells; SARS-CoV2 | 328 | QVQLVESGGGVVQPGRSLRLSCAASGF | 1407 |
| 2203 | | SARS-CoV2 | | | | RBD | Human Patient | | TFSTYAMHWVRQAPGKGLAWVALISYD | |
| | | | | | | | | | GYNKYYADSVRGRFTISRINSKNTLSLQM | |
| | | | | | | | | | NSLRAEDTAVYYCARGSAGNYYYGMDV | |
| | | | | | | | | | WGQGTTVTVSS | |
| CoV2- | Ab | SARS-CoV2 | SARS- | | SARS-CoV1, | S; non- | B-cells; SARS-CoV2 | 329 | EVQLVQSGAEVKKPGESLKISCKGSGYSF | 1408 |
| 2207 | | | CoV1 | | SARS-CoV2 | RBD | Human Patient | | TSHWIGWVRQMPGKGLEWMGIIYPG | |
| | | | | | | | | | DSDTRYSPSFQGQVTISADKSISTAYLQW | |
| | | | | | | | | | SSLKASDTAMYYCASALRERGVQLWSV | |
| | | | | | | | | | WGQGTLVTVSS | |
| CoV2- | Ab | SARS-CoV2 | SARS- | | SARS-CoV1, | S; NTD | B-cells; SARS-CoV2 | 330 | QVQLVESGGGVVQPGRSLRLSCAASGF | 1409 |
| 2210 | | | CoV1 | | SARS-CoV2 | | Human Patient | | TFSSYAMHWVRQAPGKGLEWVAVISY | |
| | | | | | | | | | DGSNKYYADSVKGRFTISRDNSKNTLYL | |
| | | | | | | | | | QMNSLRVEDTAVYYCARDQEWFRELFL | |
| | | | | | | | | | FDYWGQGTLVTVSS | |
| CoV2- | Ab | SARS-CoV2 | SARS- | | SARS-CoV1, | S; NTD | B-cells; SARS-CoV2 | 331 | EVQLVESGGGLVKPGGSLRLSCAASGFT | 1410 |
| 2212 | | | CoV1 | | SARS-CoV2 | | Human Patient | | FSSYSMNWVRQAPGKGLEWVSSISNSN | |
| | | | | | | | | | SFIYYADSMKGRFTISRDNAKNSLYLQM | |
| | | | | | | | | | NSLRAEDTAVYYCARVNGNSNWNFGSY | |
| | | | | | | | | | YYYYMDVWGKGTTVTVSS | |
| CoV2- | Ab | SARS-CoV1, | | | SARS-CoV2 | S; non- | B-cells; SARS-CoV2 | 332 | QVQLVQSGAEVKKPGSSVKVSCKASGG | 1411 |
| 2214 | | SARS-CoV2 | | | | RBD | Human Patient | | TFSSYAIIWVRQAPGQGLEWMGGIIPIF | |
| | | | | | | | | | GTTNYAQKFQGRVTITADESTSTAYVELS | |
| | | | | | | | | | SLRSEDTAVYYCARIGHFDSSGYYLDYW | |
| | | | | | | | | | GQGTLVTVSS | |
| CoV2- | Ab | SARS-CoV2 | SARS- | | SARS-CoV1, | S; NTD | B-cells; SARS-CoV2 | 333 | EVQLVESGGGLVKPGGSLRLSCAASGFT | 1412 |
| 2215 | | | CoV1 | | SARS-CoV2 | | Human Patient | | FSGYSMNWVRQAPGKGLEWVSSISSSS | |
| | | | | | | | | | SYIYYADSVKGRFTISRDNAKNSLYLQMN | |
| | | | | | | | | | SLRAEDTAVYYCARWLQLRSDYYYFGM | |
| | | | | | | | | | DVWGQGTTVTVSS | |
| CoV2- | Ab | SARS-CoV1, | | | SARS-CoV2 | S; non- | B-cells; SARS-CoV2 | 334 | QVQLVQSGAEVKKPGSSVKVSCKASGG | 1413 |
| 2216 | | SARS-CoV2 | | | | RBD | Human Patient | | TFSSYAISWVRQAPGQGLEWMGGIIPIF | |
| | | | | | | | | | GAANYAQNFQGRVTITADESTSTGYMQ | |
| | | | | | | | | | LSSLRFEDTAVYYCARTSHYDSSGSYFEY | |
| | | | | | | | | | WGQGTLVTVSS | |
| CoV2- | Ab | SARS-CoV1, | | | SARS-CoV2 | S; non- | B-cells; SARS-CoV2 | 335 | QVQLVESGGGVVQPGRSLRLSCAASGF | 1414 |
| 2218 | | SARS-CoV2 | | | | RBD | Human Patient | | TFSSYALFWVRQAPGKGLEWVAVISYD | |
| | | | | | | | | | GNNKYYADSVRGRFTISRDNSKNTLYLQ | |
| | | | | | | | | | MNSLRPEDTAVYYCARPYTGSYKSYMD | |
| | | | | | | | | | VWGKGTTVTVSS | |
| CoV2- | Ab | SARS-CoV2 | SARS- | | SARS-CoV1, | S; NTD | B-cells; SARS-CoV2 | 336 | QVQLQESGPGLVKPSETLSLTCTVSGGSI | 1415 |
| 2222 | | | CoV1 | | SARS-CoV2 | | Human Patient | | SSYYWSWIRQPPGKGLEWIGYIYYSGST | |
| | | | | | | | | | NYNPSLKSRVTISVDMSKNQFSLKLRSVT | |
| | | | | | | | | | AADTAVYYCARAPRERLQWGEYYFDYW | |
| | | | | | | | | | GQGTLVTVSS | |
| CoV2- | Ab | SARS-CoV1, | | | SARS-CoV2 | S; non- | B-cells; SARS-CoV2 | 337 | QVQLVESGGGVVQPGRSLRLSCAASGF | 1416 |
| 2224 | | SARS-CoV2 | | | | RBD | Human Patient | | TFSTYGMHWVRQAPGKGLEWVAVISY | |
| | | | | | | | | | DGSNKYYADSVKGRFTISRDNSKNTLYL | |
| | | | | | | | | | QMNSLRAEDTAMYYCAKDGSIAAADY | |
| | | | | | | | | | WGQGTLVTVSS | |
| CoV2- | Ab | SARS-CoV2 | SARS- | | SARS-CoV1, | S; non- | B-cells; SARS-CoV2 | 338 | EVQLVQSGAEVKKPGESLKISCKGSGYSF | 1417 |
| 2226 | | | CoV1 | | SARS-CoV2 | RBD | Human Patient | | TNSWIGWVRQMPGKGLEWMGIIYPG | |
| | | | | | | | | | DSDTRYSPSFQGQVTISADKSISTAYLQW | |
| | | | | | | | | | SSLKASDTAIYYCATHRCSGGFCYLAYWG | |
| | | | | | | | | | QGTLVTVSS | |
| CoV2- | Ab | SARS-Cov1, | | | SARS-CoV2 | S; non- | B-cells; SARS-CoV2 | 339 | QVQLVESGGGVVQPGRSLRLSCAASGF | 1418 |
| 2227 | | SARS-CoV2 | | | | RBD | Human Patient | | TFSSYGMHWVRQAPGKGLEWVAVIWY | |
| | | | | | | | | | DGSKKDYADSVKGRFTISRDNSKNTLYL | |
| | | | | | | | | | QMNSLRAEDTAVYYCARDQSQGAYILT | |
| | | | | | | | | | GYRGYGMDVWGQGTTVTVSS | |
| CoV2- | Ab | SARS-CoV2 | SARS- | | SARS-CoV1, | S; NTD | B-cells; SARS-CoV2 | 340 | QVQLQQWGAGLLKPSETLSLTCAVYGG | 1419 |
| 2228 | | | CoV1 | | SARS-CoV2 | | Human Patient | | SFSGHYWSWIRQPPGKGLEWIGEINHS | |
| | | | | | | | | | GSTNYNPSLKSRVTISVDTSKNQFSLKLSS | |
| | | | | | | | | | VTAADTAVYYCARPPQAARIHYYYYMD | |
| | | | | | | | | | VWGKGTTVTVSS | |
| CoV2- | Ab | SARS-CoV1, | | | SARS-CoV2 | S; RBD | B-cells; SARS-CoV2 | 34 | EVQLVESGGGLVQPGRSLRLSCAASGFT | 1420 |
| 2231 | | SARS-CoV2 | | | | | Human Patient | | FDDYAMNWVRQPPGKGLEWVSGISW | |
| | | | | | | | | | NSDSIGYADSVKGRFTISRDNAKNSLYLQ | |
| | | | | | | | | | MNSLRAEDTAMYYCAKGRGAGYTSYM | |
| | | | | | | | | | DVWGKGTTVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | SYELTQPPSVSVSPGQTASITCFGDKLG | IGHV1-24 | IGHJ4 | IGLV3-1 | IGLJ2 | 2415 | ATGFAVFGRA | 3617 | QAWDSST | Seth Zost et al., 2020 |
| 2199 | DKYACWFQQKPGQSPVLIIYQGAKRP | (Human) | (Human) | (Human) | (Human) | | AVPY | | VV | (https://www.nature.co |
| | SGIPERFSGSNSGNTATLTISGTQAMD | | | | | | | | | m/articles/s41591-020- |
| | EADYYCQAWDSSTVVFGGGTKLTVL | | | | | | | | | 0998-x) |
| CoV2- | DIQMTQSPSSLSASVGDRVTITCRASQ | IGHV3-30 | IGHJ6 | IGKV1-39 | IGKJ2 | 2416 | ARGSAGNYYY | 3618 | QQSYSTPY | Seth Zost et al., 2020 |
| 2203 | TITNYLNWYQLKSGRAPKLLIYAASSLQ | (Human) | (Human) | (Human) | (Human) | | GMDV | | T | (https://www.nature.co |
| | SGVPSRFSGSGSGTDFTLTISSLQPEDF | | | | | | | | | m/articles/s41591-020- |
| | ATYYCQQSYSTPYTFGQGTKLEIK | | | | | | | | | 0998-x) |
| CoV2- | QSVLTQPPSVSGAPGQRVTISCTGSSS | IGHV5-51 | IGHJ4 | IGLV1-40 | IGLJ3 | 2417 | ASALRERGVQ | 3619 | QSYDSSLG | Seth Zost et al., 2020 |
| 2207 | NIGAGYDVHWYQQLPGTAPKLLIFINS | (Human) | (Human) | (Human) | (Human) | | LWSV | | AL | (https://www.nature.co |
| | NRPSGVPDRFSGSKSGTSASLAITGLQ | | | | | | | | | m/articles/s41591-020- |
| | AEDEADYYCQSYDSSLGALFGGGTKLT | | | | | | | | | 0998-x) |
| | VL | | | | | | | | | |
| CoV2- | DIQMTQSPSSVSASVGDRVTITCRASQ | IGHV3-30- | IGHJ4 | IGKV1-12 | IGKJ3 | 2418 | ARDQEWFRE | 3620 | QQANSFPP | Seth Zost et al., 2020 |
| 2210 | GISSWLAWYQQKPGKAPKLLIYDASSL | 3 | (Human) | (Human) | (Human) | | LFLFDY | | T | (https://www.nature.co |
| | QSGVPSRFSGSGSGTDFTLTISSLQPED | (Human) | | | | | | | | m/articles/s41591-020- |
| | FATYYCQQANSFPPTFGPGTKVDIK | | | | | | | | | 0998-x) |
| CoV2- | EIVLTQSPAILSLSPGERATLSCRASQSV | IGHV3-21 | IGHJ6 | IGKV3-11 | IGKJ1 | 2419 | ARVNGNSN | 3621 | QQRGNW | Seth Zost et al., 2020 |
| 2212 | SSYLAWYQQKPGQAPRLLIYDTSNRAT | (Human) | (Human) | (Human) | (Human) | | WNFGSYYYYY | | WT | (https://www.nature.co |
| | GIPARFSGSGSGTDFTLTISSLEPEDFAF | | | | | | MDV | | | m/articles/s41591-020- |
| | YYCQQRGNWWTFAQGTKVEIK | | | | | | | | | 0998-x) |
| CoV2- | EIVLTQSPATLSLSPGERATLSCRASQS | IGHV1-69 | IGHJ4 | IGKV3-11 | IGKJ3 | 2420 | ARIGHFDSSG | 3622 | QHRTNWP | Seth Zost et al., 2020 |
| 2214 | VSSFLAWYQQKPGQAPRLLIYDASNR | (Human) | (Human) | (Human) | (Human) | | YYLDY | | PLFT | (https://www.nature.co |
| | PTGIPARFTGSGSGTDFTLTISSLEPEDF | | | | | | | | | m/articles/s41591-020- |
| | AVYYCQHRTNWPPLFTFGPGTKVDIK | | | | | | | | | 0998-x) |
| CoV2- | EIVMTQSPATLSVSPGERATLSCRASQ | IGHV3-21 | IGHJ6 | IGKV3-15 | IGKJ1 | 2421 | ARWLQLRSD | 3623 | QQCYNWP | Seth Zost et al., 2020 |
| 2215 | SVSNNLAWYQHKPGQAPRLLIYGAST | (Human) | (Human) | (Human) | (Human) | | YYYFGMDV | | PWT | (https://www.nature.co |
| | RATGIPARFSGSGSGTEFTLTISSLQSE | | | | | | | | | m/articles/s41591-020- |
| | DFAVYFCQQCYNWPPWTFGQGTKVE | | | | | | | | | 0998-x) |
| | IK | | | | | | | | | |
| CoV2- | EIVLTQSPATLSLSPGERATLSCRASQS | IGHV1-69 | IGHJ4 | IGKV3-11 | IGKJ4 | 2422 | ARTSHYDSSG | 3624 | HKRSNWP | Seth Zost et al., 2020 |
| 2216 | VSSYLAWYQQKPGQAPRLLIYDASNR | (Human) | (Human) | (Human) | (Human) | | SYFEY | | PSLT | (https://www.nature.co |
| | ATGIPARFSGSGSGTDFTLTISSLDPED | | | | | | | | | m/articles/s41591-020- |
| | FAVYYCHKRSNWPPSLTFGGGTKVEIK | | | | | | | | | 0998-x) |
| CoV2- | DIVMTQSPDSLAVSLGERATINCKSSQ | IGHV3-30 | IGHJ6 | IGKV4-1 | IGKJ1 | 2423 | ARPYTGSYKS | 3625 | QQYYSISW | Seth Zost et al., 2020 |
| 2218 | SVLYSSNNKNSLAWYQQKPGQPPKLLI | (Human) | (Human) | (Human) | (Human) | | YMDV | | T | (https://www.nature.co |
| | YWASTRESGVPDRFSGSGSGTDFTLTI | | | | | | | | | m/articles/s41591-020- |
| | SSLQAEDVAVYYCQQYYSISWTFGQG | | | | | | | | | 0998-x) |
| | TKVEIK | | | | | | | | | |
| CoV2- | QSALTQPASVSGSPGQSITISCTGTSSD | IGHV4-59 | IGHJ4 | IGLV2-23 | IGLJ2 | 2424 | ARAPRERLQ | 3626 | CSYAVST | Seth Zost et al., 2020 |
| 2222 | VGSYNLVSWYQQHAGKAPKLMIYEVI | (Human) | (Human) | (Human) | (Human) | | WGEYYFDY | | TYVI | (https://www.nature.co |
| | KRPSGVSNRFSGSKSGNTASLTISGLQ | | | | | | | | | m/articles/s41591-020- |
| | AEDEADYYCCSYAVSTTYVIFGGGTKLT | | | | | | | | | 0998-x) |
| | VL | | | | | | | | | |
| CoV2- | DIVMTQSPDSLAVSLGERATINCKSSQ | IGHV3-30 | IGHJ4 | IGKV4-1 | IGKJ1 | 2425 | AKDGSIAAAD | 3627 | QQYYSTP | Seth Zost et al., 2020 |
| 2224 | SVLHSSNNKDSLVWYQQKPGQPPKLL | (Human) | (Human) | (Human) | (Human) | | Y | | WT | (https://www.nature.co |
| | IYWASSRESGVPDRFSGSGSGTDFTLTI | | | | | | | | | m/articles/s41591-020- |
| | SSLQAEDVAVYYCQQYYSTPWTFGQG | | | | | | | | | 0998-x) |
| | TKVEIK | | | | | | | | | |
| CoV2- | QPVLTQPPSASASLGASVTLTCTLSSGY | IGHV5-51 | IGHJ4 | IGLV9-49 | IGLJ2 | 2426 | ATHRCSGGFC | 3628 | GADHGSG | Seth Zost et al., 2020 |
| 2226 | SNYKVDWYQQRPGKGPRFVMRVGT | (Human) | (Human) | (Human) | (Human) | | YLAY | | SNFVFVV | (https://www.nature.co |
| | GGIVGSKGDGIPDRFSVLGSGLNRYLTI | | | | | | | | | m/articles/s41591-020- |
| | KNIQEEDESDYHCGADHGSGSNFVFV | | | | | | | | | 0998-x) |
| | VFGGGTKLTVL | | | | | | | | | |
| CoV2- | DIVMTQSPLSLPVTPGEPASISCRSSQS | IGHV3-33 | IGHJ6 | IGKV2-28 | IGKJ3 | 2427 | ARDQSQGAYI | 3629 | MQALQTP | Seth Zost et al., 2020 |
| 2227 | LLHSNGYNYLDWYLQKPGQSPQFLIYL | (Human) | (Human) | (Human) | (Human) | | LTGYRGYGM | | | (https://www.nature.co |
| | GSNRASGVPDRFSGSGSGTDFILKISR | | | | | | DV | | | m/articles/s41591-020- |
| | VEAEDVGVYYCMQALQTPFTFGPGTK | | | | | | | | | 0998-x) |
| | VDIK | | | | | | | | | |
| CoV2- | EIVMTQSPATLSVSPGERATLSCRASQ | IGHV4-34 | IGHJ6 | IGKV3-15 | IGKJ4 | 2428 | ARPPQAARIH | 3630 | QQYNYWP | Seth Zost et al., 2020 |
| 2228 | SVSSNLAWYQQKPGQAPRLLIYGAST | (Human) | (Human) | (Human) | (Human) | | YYYYMDV | | PLT | (https://www.nature.co |
| | RATGIPARFSGSGSGTEFTLTISSLQSE | | | | | | | | | m/articles/s41591-020- |
| | DFAVYYCQQYNYWPPLTFGGGTKVEI | | | | | | | | | 0998-x) |
| | K | | | | | | | | | |
| CoV2- | SYVLTQPPSVSVAPGKTARITCEGNNI | IGHV3-9 | IGHJ6 | IGLV3-21 | IGLJ2 | 2429 | AKGRGAGYTS | 3631 | QVWDSSS | Seth Zost et al., 2020 |
| 2231 | GSKSVHWYQQKPGQAPVLVVYDDSG | (Human) | (Human) | (Human) | (Human) | | YMDV | | DHHVV | (https://www.nature.co |
| | RPSGIPERFSGSNSGNTATLTISRVEAG | | | | | | | | | m/articles/s41591-020- |
| | DEADYFCQVWDSSSDHHVVFGGGTK | | | | | | | | | 0998-x) |
| | LTVL | | | | | | | | | |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV1, | | | SARS-CoV2 | S; non- | B-cells; SARS-CoV2 | 342 | EVQLVESGGGLIQPGGSLRLSCAASGFIV | 1421 |
| 2235 | | SARS-CoV2 | | | | RBD | Human Patient | | SSNYMSWVRQAPGKGLEWVSVIYSGG | |
| | | | | | | | | | STYYADSVKGRFTISRDNSKNTLYLQMN | |
| | | | | | | | | | SLRAEDTAVYYCARESTQWGQGTLVTV | |
| | | | | | | | | | SS | |
| CoV2- | Ab | SARS-CoV2 | SARS- | | SARS-CoV1, | S; non- | B-cells; SARS-CoV2 | 343 | QITLKESGPPLVEPKQTLTLTCTFSGFSLT | 1422 |
| 2238 | | | CoV1 | | SARS-CoV2 | RBD | Human Patient | | TSGEAVGWIRQPPGKALEWLALIYWDD | |
| | | | | | | | | | DKHYSPSLRNRLTITRDTSKNQVVLTLTN | |
| | | | | | | | | | VDPADTGTYYCAHRAVILNFDHWGQGF | |
| | | | | | | | | | LVTVSS | |
| CoV2- | Ab | SARS-CoV1, | | | SARS-CoV2 | S; non- | B-cells; SARS-CoV2 | 344 | QVQLVESGGGVVQPGRSLRLSCAASGF | 1423 |
| 2239 | | SARS-CoV2 | | | | RBD | Human Patient | | TFSSYAMHWVRQAPGKGLEWVAVISY | |
| | | | | | | | | | DGINKYYADAVKGRFTISRDNSKNTLYL | |
| | | | | | | | | | QMNSLRAEDTAVYYCARPRSGSYYAYFD | |
| | | | | | | | | | YWGQGTLVTVSS | |
| CoV2- | Ab | SARS-CoV2 | SARS- | | SARS-Cov1, | S; RBD | B-cells; SARS-CoV2 | 345 | EVQLVESGGGLVQPGRSLRLSCAASGFT | 1424 |
| 2240 | | | CoV1 | | SARS-CoV2 | | Human Patient | | FDDYAMHWVRQAPGKGLEWVSGISW | |
| | | | | | | | | | NSGSIGYADSVKGRFTISRDNAKNSLYLQ | |
| | | | | | | | | | MNSLRAEDTALYYCAKVGYTISRQWLV | |
| | | | | | | | | | GEFDYWGQGTLVTVSS | |
| CoV2- | Ab | SARS-CoV2 | SARS- | | SARS-CoV1, | S; non- | B-cells; SARS-CoV2 | 346 | EVQLVQSGAEVKKPGESLKISCKGSGYSF | 1425 |
| 2241 | | | CoV1 | | SARS-CoV2 | RBD | Human Patient | | TSYWIDWVRQMPGKGLEWMGIIYPGD | |
| | | | | | | | | | SDTRYSPSFQGQVTISADKSTSTAYLQW | |
| | | | | | | | | | SSLKASDTAMYYCARRGEAAGIWYFDL | |
| | | | | | | | | | WGRGTLVTVSS | |
| CoV2- | Ab | SARS-CoV2 | SARS- | | SARS-CoV1, | S; RBD | B-cells; SARS-CoV2 | 347 | QLQLQESGPGLVKPSETLPLTCTVSGGSI | 1426 |
| 2243 | | | CoV1 | | SARS-CoV2 | | Human Patient | | SSSSYYWGWIRQPPGKGLQWIGNIYYS | |
| | | | | | | | | | GSTYYNSSLKSRVTISVDTSKNQFSLKLSS | |
| | | | | | | | | | VTAADTAVYYCARQSRGYSYAWSFDYW | |
| | | | | | | | | | GQGTLVTVSS | |
| CoV2- | Ab | SARS-CoV2 | SARS- | | SARS-CoV1, | S; non- | B-cells; SARS-CoV2 | 348 | QVQLVESGGGVVQPGRSLRLSCAASGF | 1427 |
| 2245 | | | CoV1 | | SARS-CoV2 | RBD | Human Patient | | TFSNYGIHWVRQAPGQGLEWVAGIWY | |
| | | | | | | | | | DGSNKYYVDSVKGRFTISRDNSKNTLYL | |
| | | | | | | | | | QMNSLRAEDTAVYYCAGSSGEGGLYYY | |
| | | | | | | | | | YGMDVWGQGTTVTVSS | |
| CoV2- | Ab | SARS-CoV2 | SARS- | | SARS-CoV1, | S; non- | B-cells; SARS-CoV2 | 349 | QVQLVESGGGVVQPGRSLRLSCAASGF | 1428 |
| 2248 | | | CoV1 | | SARS-CoV2 | RBD | Human Patient | | TFDSYGVHWVRQAPGKGLEWVAVISYD | |
| | | | | | | | | | GSNKHYADSVKGRFTISRDNSKNTLYVQ | |
| | | | | | | | | | MNSLRTEDTAVYYCARDSGGNYGDSYF | |
| | | | | | | | | | DYWGQGTLVTVSS | |
| CoV2- | Ab | SARS-CoV1, | | | SARS-CoV2 | S; non- | B-cells; SARS-CoV2 | 350 | QVQLVESGGGVVQPGRSLRLSCAASGF | 1429 |
| 2250 | | SARS-CoV2 | | | | RBD | Human Patient | | TFSTYAMHWVRQAPGKGLAWVALISYD | |
| | | | | | | | | | GYNKYYADSVRGRFTISRINSKNTLSLQM | |
| | | | | | | | | | NSLRAEDTAVYYCARGSAGNYYYGMDV | |
| | | | | | | | | | WGQGTTVTVSS | |
| CoV2- | Ab | SARS-CoV2 | SARS- | | SARS-CoV1, | S; NTD | B-cells; SARS-CoV2 | 351 | QVTLKESGPVLVKPTETLTLTCAVSGFSLS | 1430 |
| 2251 | | | CoV1 | | SARS-CoV2 | | Human Patient | | NAKMGVSWIRQPPGKALEWLAHIFSN | |
| | | | | | | | | | DEKAYSTSLKTRLTISKDTSKSQVVLTVTN | |
| | | | | | | | | | MDPVDTATYYCARIVLGASGTYPSPGFD | |
| | | | | | | | | | PWGQGTLVTVSS | |
| CoV2- | Ab | SARS-CoV2 | SARS- | | SARS-CoV1, | S; RBD | B-cells; SARS-CoV2 | 352 | EVQLVESGGGLVQPGGSLRLSCAASGFT | 1431 |
| 2253 | | | CoV1 | | SARS-CoV2 | | Human Patient | | FSSYWMHWVRQVPGKGLVWVSRINSD | |
| | | | | | | | | | GSSTSYADSVKGRFTISRDNAKNTLYLE | |
| | | | | | | | | | MNSLRAQDTAVYYCAGSPWLRGDIDY | |
| | | | | | | | | | WGQGTLVTVSS | |
| CoV2- | Ab | SARS-CoV1, | | | SARS-CoV2 | S; RBD | B-cells; SARS-CoV2 | 353 | QVQLVQSGSELTKPGASVKVSCKASGYT | 1432 |
| 2256 | | SARS-CoV2 | | | | | Human Patient | | FTSYAMNWVRQAPGQGLEWMGWINT | |
| | | | | | | | | | DTGNPTYAQGFTGRFVFSLDTSVSTAYL | |
| | | | | | | | | | QISSLKAEDTAVYYCARDPSYCSSTRCYT | |
| | | | | | | | | | VGWFDPWGQGTLVTVSS | |
| CoV2- | Ab | SARS-CoV2 | SARS- | | SARS-CoV1, | S; NTD | B-cells; SARS-CoV2 | 354 | QVQLQESGPGLVKPSQTLSLTCTVSGDSI | 1433 |
| 2257 | | | CoV1 | | SARS-CoV2 | | Human Patient | | NSGNYYWSWIRQPAGKGLEWIGRMFT | |
| | | | | | | | | | SGSTNYNPSLTSRVTMSIDTSKNQFSLNL | |
| | | | | | | | | | NSVTAADTAMYYCARGHVAAWESCYY | |
| | | | | | | | | | WGQGILVTVSS | |
| CoV2- | Ab | SARS-CoV1, | | SARS-CoV2 | | S; RBD | B-cells; SARS-CoV2 | 355 | EVQLVESGGGVVRPGGSLRLSCAASGFT | 1434 |
| 2258 | | SARS-CoV2 | | | | | Human Patient | | LDDYGLSWVRHAPGKGLEWVSGINWN | |
| | | | | | | | | | GGRTAYADSVKGRFTISRDNAKNSLYLQ | |
| | | | | | | | | | MNSLRAEDTALYHCARARGPSEQYYDLL | |
| | | | | | | | | | TGYYDAFDIWGQGTMVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | DIQMTQSPSTLSASVGDRVTITCRASH | IGHV3-53 | IGHJ4 | IGKV1-5 | IGKJ1 | 2430 | ARESTQ | 3632 | QQYNTYS | Seth Zost et al., 2020 |
| 2235 | SISSWLAWYQQKPGKAPKLLIYKASSL | (Human) | (Human) | (Human) | (Human) | | | | QT | (https://www.nature.co |
| | ESGVPSRFSGSGSGTEFTLTISSLQPDD | | | | | | | | | m/articles/s41591-020- |
| | FATYYCQQYNTYSQTFGQGTKVEIK | | | | | | | | | 0998-x) |
| CoV2- | FYELTQPPSVSVSPGQTAGITCSGDKL | IGHV2-5 | IGHJ4 | IGLV3-1 | IGLJ3 | 2431 | AHRAVILNFD | 3633 | QAWDND | Seth Zost et al., 2020 |
| 2238 | GHKYAYWYQQKPGQSPILLIYQDDKR | (Human) | (Human) | (Human) | (Human) | | H | | AGVV | (https://www.nature.co |
| | PSGIPERFSGSNSGTIATLTISGTQPVD | | | | | | | | | m/articles/s41591-020- |
| | EADYYCQAWDNDAGVVFGGGTKLTV | | | | | | | | | 0998-x) |
| | L | | | | | | | | | |
| CoV2- | DIQMTQSPSSLSASVGDRVTITCRASQ | IGHV3-30- | IGHJ4 | IGKV1-16 | IGKJ4 | 2432 | ARPRSGSYYA | 3634 | QQYNSHP | Seth Zost et al., 2020 |
| 2239 | GISNYLAWFQQKPGKAPKSLIYAASSL | 3 | (Human) | (Human) | (Human) | | YFDY | | PT | (https://www.nature.co |
| | QSGVPSKFSGSGSGTDFTLTISSLQPED | (Human) | | | | | | | | m/articles/s41591-020- |
| | VATYYCQQYNSHPPTFGGGTKVEIK | | | | | | | | | 0998-x) |
| CoV2- | QSALTQPPSASGSPGQSVTISCTGTSS | IGHV3-9 | IGHJ4 | IGLV2-8 | IGLJ2 | 2433 | AKVGYTISRQ | 3635 | SAYAGSNN | Seth Zost et al., 2020 |
| 2240 | DVGTYNYVSWYQQHPGKAPKLMIYE | (Human) | (Human) | (Human) | (Human) | | WLVGEFDY | | LV | (https://www.nature.co |
| | VSKRPSGVPDRFSGSKSGNTASLTVSG | | | | | | | | | m/articles/s41591-020- |
| | LQAEDEAEYYCSAYAGSNNLVFGGGT | | | | | | | | | 0998-x) |
| | KLTVL | | | | | | | | | |
| CoV2- | QPVLTQPPSASASLGASVTLTCTLSSGY | IGHV5-51 | IGHJ2 | IGLV9-49 | IGLJ2 | 2434 | ARRGEAAGI | 3636 | GADHGSG | Seth Zost et al., 2020 |
| 2241 | SNYKVDWYQQRPGKGPRFVMRVGT | (Human) | (Human) | (Human) | (Human) | | WYFDL | | SNFEYVV | (https://www.nature.co |
| | GGIVGSKGDGIPDRFSVLGSGLNRYLTI | | | | | | | | | m/articles/s41591-020- |
| | KNIQEEDESDYHCGADHGSGSNFEYV | | | | | | | | | 0998-x) |
| | VFGGGTKLTVL | | | | | | | | | |
| CoV2- | QSALTQPASVSGSHGQSITISCTGTSSD | IGHV4-39 | IGHJ4 | IGLV2-23 | IGLJ2 | 2435 | ARQSRGYSYA | 3637 | CSYAGIVL | Seth Zost et al., 2020 |
| 2243 | VGSYNLVSWYQQHPGKAPKLMIYEVS | (Human) | (Human) | (Human) | (Human) | | WSFDY | | | (https://www.nature.co |
| | KRPSGVSNRFSGSTSGNTASLTISGLQ | | | | | | | | | m/articles/s41591-020- |
| | AEDEADYYCCSYAGIVLFGGGTKVTVL | | | | | | | | | 0998-x) |
| CoV2- | SYELTQPPSVSVSPGQTTRITCSGDALP | IGHV3-33 | IGHJ6 | IGLV3-25 | IGLJ3 | 2436 | AGSSGEGGLY | 3638 | QSGDSSGT | Seth Zost et al., 2020 |
| 2245 | KQYGYWYQQKAGQAPILVIYKDSERP | (Human) | (Human) | (Human) | (Human) | | YYYGMDV | | YVV | (https://www.nature.co |
| | SGIPERFSGSSSGTTVTLTISGVQAEDE | | | | | | | | | m/articles/s41591-020- |
| | AHYYCQSGDSSGTYVVFGGGTKLTVL | | | | | | | | | 0998-x) |
| CoV2- | SYELTQPPSVSVSPGQTARITCSGDALS | IGHV3-30 | IGHJ4 | IGLV3-25 | IGLJ3 | 2437 | ARDSGGNYG | 3639 | QSVDRSGT | Seth Zost et al., 2020 |
| 2248 | KQYAYWYQQKPGQAPVLVIYKDSERP | (Human) | (Human) | (Human) | (Human) | | DSYFDY | | YFNWV | (https://www.nature.co |
| | SGIPERFSGSSSGTTVTLTISGVQAEDE | | | | | | | | | m/articles/s41591-020- |
| | ADYYCQSVDRSGTYFNWVFGGGTKLT | | | | | | | | | 0998-x) |
| | VL | | | | | | | | | |
| CoV2- | DIQMTQSPSSLSASVGDRVTITCRASQ | IGHV3-30 | IGHJ6 | IGKV1-39 | IGKJ2 | 2438 | ARGSAGNYYY | 3640 | QQSYSTPY | Seth Zost et al., 2020 |
| 2250 | TITNYLNWYQLKSGRAPKLLIYAASSLQ | (Human) | (Human) | (Human) | (Human) | | GMDV | | T | (https://www.nature.co |
| | SGVPSRFSGSGSGTDFTLTISSLQPEDF | | | | | | | | | m/articles/s41591-020- |
| | ATYYCQQSYSTPYTFGQGTKLEIK | | | | | | | | | 0998-x) |
| CoV2- | QTVVTQEPSLTVSPGGTVTLTCASSAG | IGHV2-26 | IGHJ5 | IGLV7-43 | IGLJ3 | 2439 | ARIVLGASGT | 3641 | LLYYGGA | Seth Zost et al., 2020 |
| 2251 | AVTSGYYPNWFQQKPGQAPRALIYST | (Human) | (Human) | (Human) | (Human) | | YPSPGFDP | | WV | (https://www.nature.co |
| | ANKHSWTLARFSGSLLGGKAALTLSG | | | | | | | | | m/articles/s41591-020- |
| | VQPEDEAEYYCLLYYGGAWVFGGGTK | | | | | | | | | 0998-x) |
| | LTVL | | | | | | | | | |
| CoV2- | NFMLTQPHSVSESPGKTVTISCTGSSG | IGHV3-74 | IGHJ4 | IGLV6-57 | IGLJ2 | 2440 | AGSPWLRGDI | 3642 | QSYDGSN | Seth Zost et al., 2020 |
| 2253 | SIASNYVQWYQQRPGSAPTTVIYEDN | (Human) | (Human) | (Human) | (Human) | | DY | | HAVV | (https://www.nature.co |
| | QRPSGVPDRFSGSIDSSSNSASLTISGL | | | | | | | | | m/articles/s41591-020- |
| | MTEDEADYYCQSYDGSNHAVVFGGG | | | | | | | | | 0998-x) |
| | TKLTVL | | | | | | | | | |
| CoV2- | SYVLTQPPSVSVAPGKTARITCGGNNI | IGHV7-4- | IGHJ5 | IGLV3-21 | IGLJ2 | 2441 | ARDPSYCSST | 3643 | QVWDSSS | Seth Zost et al., 2020 |
| 2256 | GSKNVHWYQQKPGQAPVVVVYDDS | 1 | (Human) | (Human) | (Human) | | RCYTVGWFD | | DPVV | (https://www.nature.co |
| | DRPSGIPERFSGSNSGNTATLTISRVEA | (Human) | | | | | P | | | m/articles/s41591-020- |
| | GDEADYYCQVWDSSSDPVVFGGGTK | | | | | | | | | 0998-x) |
| | LTVL | | | | | | | | | |
| CoV2- | NIVMTQSPLSLPVTPGEPASISCRSSQS | IGHV4-61 | IGHJ4 | IGKV2-28 | IGKJ1 | 2442 | ARGHVAAWE | 3644 | MQALQTP | Seth Zost et al., 2020 |
| 2257 | LLNSNGLTYLDWYLQKPGQSPQLLIYF | (Human) | (Human) | (Human) | (Human) | | SCYY | | QT | (https://www.nature.co |
| | VSNRASGVSDRFSGSGSGADFTLTISR | | | | | | | | | m/articles/s41591-020- |
| | VEAEDVGVYYCMQALQTPQTFGQGT | | | | | | | | | 0998-x) |
| | KVEIK | | | | | | | | | |
| CoV2- | EIVLTQSPGTLSLSPGERATLSCGASQSI | IGHV3-20 | IGHJ3 | IGKV3-20 | IGKJ4 | 2443 | ARARGPSEQY | 3645 | QQYGGSLT | Seth Zost et al., 2020 |
| 2258 | SSSYLAWYQQKPGQAPRLLIYGASSRA | (Human) | (Human) | (Human) | (Human) | | YDLLTGYYDA | | | (https://www.nature.co |
| | AGIPDRFSGSGSGTDFTLTISRLEPEDF | | | | | | FDI | | | m/articles/s41591-020- |
| | AVYFCQQYGGSLTFGGGTKVEIK | | | | | | | | | 0998-x) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV2 | SARS- | | SARS-CoV1, | S; NTD | B-cells; SARS-CoV2 | 356 | EVQLLESGGGLVHPGGSLRLSCAASGFT | 1435 |
| 2260 | | | CoV1 | | SARS-CoV2 | | Human Patient | | FSSYALSWVRQAPGKGLEWVSAISGSG | |
| | | | | | | | | | GSTYYADSVKGRFTISRDNSKNTLYLQM | |
| | | | | | | | | | NRLRAEDTAVYYCAQMGPLGSTSSAAD | |
| | | | | | | | | | YWGQGTLVTVSS | |
| CoV2- | Ab | SARS-CoV2 | SARS- | SARS-CoV2 | | S; non- | B-cells; SARS-CoV2 | 357 | QVQLVQSGAEVKKPGASVKVSCKASGY | 1436 |
| 2262 | | | CoV1 | (weak) | | RBD | Human Patient | | TFTSYDINWVRQATGQGLEWMGWMN | |
| | | | | | | | | | PNSGNTGYAQKFQGRVTMTRNTSISTA | |
| | | | | | | | | | YMELSSLRSEDTALYYCAREARYFDWIFE | |
| | | | | | | | | | GSDYYYYGMDVWGQGTTVTVSS | |
| CoV2- | Ab | SARS-CoV2 | SARS- | | SARS-CoV1, | S; NTD | B-cells; SARS-CoV2 | 358 | EVQLVESGGGLVQPGGSLRLSCEASGFT | 1437 |
| 2263 | | | CoV1 | | SARS-CoV2 | | Human Patient | | FSSSEINWVRQAPGKGLEWVSHISSSGSI | |
| | | | | | | | | | IYYADSVKGRFTISRDNAKNSLYLQMNSL | |
| | | | | | | | | | RAEDTAVYYCARRSYRSSWYYYYGMDV | |
| | | | | | | | | | WGQGTTVTVSS | |
| CoV2- | Ab | SARS-CoV1, | | | SARS-CoV2 | S; non- | B-cells; SARS-CoV2 | 359 | EVQLVESGGGLVQPGRSLRLSCTASGFIF | 1438 |
| 2266 | | SARS-CoV2 | | | | RBD | Human Patient | | GDYAMGWVRQAPGKGLEWVGFIRGK | |
| | | | | | | | | | AYDGTTEYAASVKGRFTISRDDSKYIAHL | |
| | | | | | | | | | QMNSLKTEDTAVYYCIRDYDFWGGYYY | |
| | | | | | | | | | HPLRAFDIWGRGTMVTVSS | |
| CoV2- | Ab | SARS-CoV2 | SARS- | SARS-CoV2 | | S; RBD | B-cells; SARS-CoV2 | 360 | QITLKESGPTLVKPTQTLTLTCTFSGFSLS | 1439 |
| 2268 | | | CoV1 | | | | Human Patient | | TSGVGVGWIRQPPGKALEWLALIYWD | |
| | | | | | | | | | DDKRYSPSLKSRLSITKDTSKNQVVLTMT | |
| | | | | | | | | | NMDPVDTGTYYCARHQIVVLFDMWG | |
| | | | | | | | | | QGTRVTVSS | |
| CoV2- | Ab | SARS-CoV1, | | | SARS-CoV2 | S; non- | B-cells; SARS-CoV2 | 361 | QVQLVQSGAEVKKPGSSVKVSCKASGG | 1440 |
| 2270 | | SARS-CoV2 | | | | RBD | Human Patient | | TFSSYAISWVRQAPGQGLEWMGGIIPIF | |
| | | | | | | | | | GTANYAQKFQGRVTITADESTSTAYMEL | |
| | | | | | | | | | SSLRSEDTAVYYCAITYYYDSSGYWWDD | |
| | | | | | | | | | WGQGTLVTVSS | |
| CoV2- | Ab | SARS-CoV1, | | | SARS-CoV2 | S; non- | B-cells; SARS-CoV2 | 362 | QLQLQESGPGLVKPSETLSLTCTVSGGSI | 1441 |
| 2273 | | SARS-CoV2 | | | | RBD | Human Patient | | SSSSYYWGWIRQPPGKGLEWIGTIYYSG | |
| | | | | | | | | | STYYNPSLKSRVTISVDTSKKQFSLKLSSV | |
| | | | | | | | | | TAADTAVYYCAGEEVRGVKLYYYYAMD | |
| | | | | | | | | | VWGQGTTVTVSS | |
| CoV2- | Ab | SARS-CoV1, | | | SARS-CoV2 | S; non- | B-cells; SARS-CoV2 | 363 | EVQLVESGGRLVLPGGSLRLSCAASGFTF | 1442 |
| 2274 | | SARS-CoV2 | | | | RBD | Human Patient | | SVYEMNWVRQAPGKGLEWLSYIGTSGS | |
| | | | | | | | | | PIYYADSVKGRFTVSRDNAKNSLYLQMN | |
| | | | | | | | | | SLRVEDTALYYCARDRGWNYGLDYWG | |
| | | | | | | | | | QGTLVTVSS | |
| CoV2- | Ab | SARS-CoV2 | SARS- | | SARS-CoV1, | S; RBD | B-cells; SARS-CoV2 | 364 | QVQLVESGGGVVQPGRSLRLSCAASGF | 1443 |
| 2277 | | | CoV1 | | SARS-CoV2 | | Human Patient | | TFSSYAMHWVRQAPGKGLEWVALISSD | |
| | | | | | | | | | GGNKFYADSVKGRFTISRDNSKNTLYLQ | |
| | | | | | | | | | MNSLRAEDTAVYYCARDVPTTVTAFTVF | |
| | | | | | | | | | TYWGQGTLVTVSS | |
| CoV2- | Ab | SARS-CoV1, | | | SARS-CoV2 | S; non- | B-cells; SARS-CoV2 | 365 | QVQLVESGGGVVQPGRSLRLSCAASGF | 1444 |
| 2281 | | SARS-CoV2 | | | | RBD | Human Patient | | AFSNYAIHWVRQAPGKGLEWVAVISYD | |
| | | | | | | | | | GNNKDYADSVKGRFTISRDNSKNTLYLQ | |
| | | | | | | | | | MNSLRAEDTAVYYCARVPVMVRGVYF | |
| | | | | | | | | | DYWGQGTLVTVSS | |
| CoV2- | Ab | SARS-CoV1, | | | SARS-CoV2 | S; RBD | B-cells; SARS-CoV2 | 366 | QVQLVQSGAEVKKPGASVKVSCKVSGY | 1445 |
| 2287 | | SARS-CoV2 | | | | | Human Patient | | TFTSYDINWVRQATGQGLEWMGWMN | |
| | | | | | | | | | PNSGHTGYAQKFQGRVTMTRNTSISTA | |
| | | | | | | | | | YMELSSLRYEDTAVYYCARGYGLTYYMD | |
| | | | | | | | | | VWGKGTTVTVSS | |
| CoV2- | Ab | SARS-CoV2 | SARS- | SARS-CoV2 | | S; RBD | B-cells; SARS-CoV2 | 367 | QVQLAQSGAEVKKPGASVKVSCKAAGY | 1446 |
| 2290 | | | CoV1 | (weak) | | | Human Patient | | TFTSYDINWVRQATGQGLEWMGWMN | |
| | | | | | | | | | PNSGNAGYAQKFQGRVTMTRDTSISTA | |
| | | | | | | | | | YMELSSLRSEDTAVYYCARMRSGWPTH | |
| | | | | | | | | | GRPDDFWGRGTLVTVSS | |
| CoV2- | Ab | SARS-CoV2 | SARS- | | SARS-CoV1, | S; NTD | B-cells; SARS-CoV2 | 368 | EVQLLQSGGGLVQPGGSLRLSCAASGFT | 1447 |
| 2293 | | | CoV1 | | SARS-CoV2 | | Human Patient | | FRNYAMSWVRQAPGKGLEWVSAISGS | |
| | | | | | | | | | GGTTYYADSVKGRFTISRDNSKNTLYLQ | |
| | | | | | | | | | MNSLRAEDTAVYYCAKNERITMLVVVTL | |
| | | | | | | | | | FDYWGQGTLVTVSS | |
| CoV2- | Ab | SARS-CoV2 | SARS- | | SARS-CoV1, | S; non- | B-cells; SARS-CoV2 | 369 | EVQLVESGGGLVQPGGSLRLSCAASGFT | 1448 |
| 2296 | | | CoV1 | | SARS-CoV2 | RBD | Human Patient | | VSGNYMSWVRQAPGKGLEWVSVIYSG | |
| | | | | | | | | | GSTYYADSVKGRFTISRDNSKNTLYLQM | |
| | | | | | | | | | NSPRAEDTAVYYCARDPSAYYDILTGYSG | |
| | | | | | | | | | DVWGKGTTVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | SYELTQPPSVSVSPGQTASITCSGDKM | IGHV3-23 | IGHJ4 | IGLV3-1 | IGLJ2 | 2444 | AQMGPLGST | 3646 | QAWDSSL | Seth Zost et al., 2020 |
| 2260 | GDKYACWYQQKPGQSPVVVIYEDNK | (Human) | (Human) | (Human) | (Human) | | SSAADY | | VV | (https://www.nature.co |
| | RPSGIPERFSGSNSGNTATLTISGTQA | | | | | | | | | m/articles/s41591-020- |
| | MDEADYYCQAWDSSLVVFGGGTKLT | | | | | | | | | 0998-x) |
| | VL | | | | | | | | | |
| CoV2- | QSALTQPPSVSGSPGQSVTISCTGTSS | IGHV1-8 | IGHJ6 | IGLV2-18 | IGLJ3 | 2445 | AREARYFDWI | 3647 | SSYTSSSL | Seth Zost et al., 2020 |
| 2262 | DVGSYNRVSWYQQPPGTAPKLMIYE | (Human) | (Human) | (Human) | (Human) | | FEGSDYYYYG | | RV | (https://www.nature.co |
| | VSNRPSGVPDRFSGSKSGNTASLTISG | | | | | | MDV | | | m/articles/s41591-020- |
| | LQAEDEADYYCSSYTSSSLRVFGGGTK | | | | | | | | | 0998-x) |
| | LTVL | | | | | | | | | |
| CoV2- | DIQLTQSPSFLSASVGDRVTITCRASQG | IGHV3-48 | IGHJ6 | IGKV1-9 | IGKJ1 | 2446 | ARRSYRSSWY | 3648 | QQLNSYPV | Seth Zost et al., 2020 |
| 2263 | ISSYLAWYQQKPGKAPKLLIYAASTLQS | (Human) | (Human) | (Human) | (Human) | | YYYGMDV | | T | (https://www.nature.co |
| | GVPSRFSGSGSGTEFTLTISSLQPEDFA | | | | | | | | | m/articles/s41591-020- |
| | TYYCQQLNSYPVTFGQGTKVEIK | | | | | | | | | 0998-x) |
| CoV2- | QSALTQPRSVSGSPGQSVTISCTGTSS | IGHV3-49 | IGHJ3 | IGLV2-11 | IGLJ1 | 2447 | IRDYDFWGG | 3649 | CSYAGSYT | Seth Zost et al., 2020 |
| 2266 | DVGGYNYVSWYQQHPGKAPKLMIYD | (Human) | (Human) | (Human) | (Human) | | YYYHPLRAFD | | YV | (https://www.nature.co |
| | VTKRPSGVPERFSGSKSGNTASLTISGL | | | | | | I | | | m/articles/s41591-020- |
| | QAEDEADYSCCSYAGSYTYVFGTGTKV | | | | | | | | | 0998-x) |
| | TVL | | | | | | | | | |
| CoV2- | QSALTQPASVSGSPGQSITISCTGTGSD | IGHV2-5 | IGHJ3 | IGLV2-14 | IGLJ2 | 2448 | ARHQIVVLFD | 3650 | SSYTSSST | Seth Zost et al., 2020 |
| 2268 | VGGSNYVSWYQQHPGKAPKLMIYDV | (Human) | (Human) | (Human) | (Human) | | M | | LVL | (https://www.nature.co |
| | SNRPSGVSNRFSGSKSGNTASLTISGL | | | | | | | | | m/articles/s41591-020- |
| | QAEDEADYYCSSYTSSSTLVLFGGGTKL | | | | | | | | | 0998-x) |
| | SVL | | | | | | | | | |
| CoV2- | EIVLTQSPATLSLSPGERATLSCRASQS | IGHV1-69 | IGHJ4 | IGKV3-11 | IGKJ2 | 2449 | AITYYYDSSG | 3651 | QQRSNWP | Seth Zost et al., 2020 |
| 2270 | VSSFLAWYQQKPGQAPRLLIYDASNR | (Human) | (Human) | (Human) | (Human) | | YWWDD | | PSYT | (https://www.nature.co |
| | ATGIPARFSGSGSGTDFTLTISSLEPEDF | | | | | | | | | m/articles/s41591-020- |
| | AVYYCQQRSNWPPSYTFGQGTKLEIK | | | | | | | | | 0998-x) |
| CoV2- | QSALTQPASVSGSPGQSITISCTGTSSD | IGHV4-39 | IGHJ6 | IGLV2-14 | IGLJ3 | 2450 | AGEEVRGVKL | 3652 | SSYTSIST | Seth Zost et al., 2020 |
| 2273 | IGGYNYVSWYQQHPGKAPKLMIYDVS | (Human) | (Human) | (Human) | (Human) | | YYYYAMDV | | WV | (https://www.nature.co |
| | NRPSGVSNRFSGSKSGNTASLTISGLQ | | | | | | | | | m/articles/s41591-020- |
| | AEDEADYYCSSYTSISTWVFGGGTKVT | | | | | | | | | 0998-x) |
| | VL | | | | | | | | | |
| CoV2- | SYELTQPSSVSVSPGQTARITCSGDALP | IGHV3-48 | IGHJ4 | IGLV3-10 | IGLJ2 | 2451 | ARDRGWNYG | 3653 | FSMDSSG | Seth Zost et al., 2020 |
| 2274 | KRYAYWYRQKSGQAPVLVIHEDSKRP | (Human) | (Human) | (Human) | (Human) | | LDY | | DLRV | (https://www.nature.co |
| | SGIPERFSGSTSGTMATLTITGAQLEDE | | | | | | | | | m/articles/s41591-020- |
| | ADYYCFSMDSSGDLRVFGGGTKLTVL | | | | | | | | | 0998-x) |
| CoV2- | DIQMTQSPSSLSASVGDRVTITCRASQ | IGHV3-30 | IGHJ4 | IGKV1-39 | IGKJ4 | 2452 | ARDVPTTVTA | 3654 | QQSYSTPG | Seth Zost et al., 2020 |
| 2277 | SISTYLNWYQQKPGKAPKLLIYAASSLQ | (Human) | (Human) | (Human) | (Human) | | FTVFTY | | LT | (https://www.nature.co |
| | SGVPSRFSGSGSGTDFTLTISSLQPEDY | | | | | | | | | m/articles/s41591-020- |
| | ATYYCQQSYSTPGLTFGGGTKVEIK | | | | | | | | | 0998-x) |
| CoV2- | QSALTQPASVSGSPGQSITISCTGTSSD | IGHV3-30- | IGHJ4 | IGLV2-14 | IGLJ2 | 2453 | ARVPVMVRG | 3655 | ISYTSSRT | Seth Zost et al., 2020 |
| 2281 | VVGYNYVSWYQQHPGKAPKLMIYDV | 3 | (Human) | (Human) | (Human) | | VYFDY | | LV | (https://www.nature.co |
| | RNRPSGVSNRFSGSKSGNTASLTISGL | (Human) | | | | | | | | m/articles/s41591-020- |
| | QAEDEADYFCISYTSSRTLVFGGGTKLT | | | | | | | | | 0998-x) |
| | VL | | | | | | | | | |
| CoV2- | SYVLTQPPSVSVAPGKTARITCGGNNI | IGHV1-8 | IGHJ6 | IGLV3-21 | IGLJ2 | 2454 | ARGYGLTYY | 3656 | QVWDSSY | Seth Zost et al., 2020 |
| 2287 | GSKNVHWYQQKPGQAPVLVVYDDS | (Human) | (Human) | (Human) | (Human) | | MDV | | YHPVV | (https://www.nature.co |
| | DRPSGIPERFSGSNSGNTATLTISRVEA | | | | | | | | | m/articles/s41591-020- |
| | GDEADYYCQVWDSSYYHPVVFGGGT | | | | | | | | | 0998-x) |
| | KLTVL | | | | | | | | | |
| CoV2- | QSVLTQPPSASGTPGQRVTISCSGSNS | IGHV1-8 | IGHJ4 | IGLV1-44 | IGLJ2 | 2455 | ARMRSGWPT | 3657 | LVWDDSL | Seth Zost et al., 2020 |
| 2290 | NIGSYTVNWYQQFPGTAPKLLIYDNN | (Human) | (Human) | (Human) | (Human) | | HGRPDDF | | NGLV | (https://www.nature.co |
| | QRTSGVPDRFSGSKSGTSASLAISGLQ | | | | | | | | | m/articles/s41591-020- |
| | SEDEANYYCLVWDDSLNGLVFGGGTK | | | | | | | | | 0998-x) |
| | LTVL | | | | | | | | | |
| CoV2- | QSVLTQPPSVSAAPGQKVTISCSGSSS | IGHV3-23 | IGHJ4 | IGLV1-51 | IGLJ2 | 2456 | AKNERITMLV | 3658 | GTWDSSLS | Seth Zost et al., 2020 |
| 2293 | NIGNNYISWYQQLPGTAPKLLIYDNNK | (Human) | (Human) | (Human) | (Human) | | VVTLFDY | | AVV | (https://www.nature.co |
| | RPSGIPDRFSGSKSGTSATLGITGLQTG | | | | | | | | | m/articles/s41591-020- |
| | DEADYYCGTWDSSLSAVVFGGGTKLT | | | | | | | | | 0998-x) |
| | VL | | | | | | | | | |
| CoV2- | DIQMTQSPSTLSASVGDRVTITCRASQ | IGHV3-66 | IGHJ6 | IGKV1-5 | IGKJ1 | 2457 | ARDPSAYYDI | 3659 | QQYSIYSW | Seth Zost et al., 2020 |
| 2296 | SINSWLAWYQQKPGKAPKLLIYKASSL | (Human) | (Human) | (Human) | (Human) | | LTGYSGDV | | T | (https://www.nature.co |
| | ESGVPSRFSGSGSGTEFTLTISSLQPDD | | | | | | | | | m/articles/s41591-020- |
| | FATYYCQQYSIYSWTFGQGTKVEIK | | | | | | | | | 0998-x) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV2 | SARS- | | SARS-CoV1, | S; RBD | B-cells; SARS-CoV2 | 370 | QVQLVESGGGVVQPGRSLRLSCAASGF | 1449 |
| 2299 | | | CoV1 | | SARS-CoV2 | | Human Patient | | SFSSYVMNWVRQAPGKGLEWVAVISY | |
| | | | | | | | | | DGSSKYYADSVKGRFTISRDNSKNTLYLQ | |
| | | | | | | | | | MNSLRAEDTAVYYCARDIDSGYDPTPVF | |
| | | | | | | | | | DYWGQGTLVTVSS | |
| CoV2- | Ab | SARS-CoV2 | SARS- | | SARS-CoV1, | S; non- | B-cells; SARS-CoV2 | 371 | QVQLQESGPGLVKPSGTLSLTCAVSGGS | 1450 |
| 2300 | | | CoV1 | | SARS-CoV2 | RBD | Human Patient | | ISSSNWWTWVRQPPGKGLEWIGEIYHS | |
| | | | | | | | | | GSTNYNPSLKSRVTISVDKSKNQFSLKLSS | |
| | | | | | | | | | VTAADTAVYYCASRWGDYFDSSGAYDS | |
| | | | | | | | | | WGQGTLVTVSS | |
| CoV2- | Ab | SARS-CoV2 | SARS- | | SARS-CoV1, | S; RBD | B-cells; SARS-CoV2 | 372 | EVQLVESGGGLVKPGGSLRLSCAASGFT | 1451 |
| 2304 | | | CoV1 | | SARS-CoV2 | | Human Patient | | FSSYSMNWVRQAPGKGLEWVSCISSSS | |
| | | | | | | | | | SFIYYADSVKGRFTISRDNAKNSLYLQMN | |
| | | | | | | | | | SLRAEDTAVYYCARDPVWVDGELLSGGI | |
| | | | | | | | | | PFDYWGQGTLVTVSS | |
| CoV2- | Ab | SARS-CoV2 | SARS- | | SARS-CoV1, | S; NTD | B-cells; SARS-CoV2 | 373 | QLQLQESGPGLVKPSETLSLTCTVSGGSI | 1452 |
| 2305 | | | CoV1 | | SARS-CoV2 | | Human Patient | | RSSSYYWGWIRQPPGKGLEWIASIYYSG | |
| | | | | | | | | | STYYNSSLKSRVTISVDTSKNQFSLKVNS | |
| | | | | | | | | | MTAADTAVYYCAILWRGSSWADRHYYY | |
| | | | | | | | | | YSMDVWGQGTTVTVSS | |
| CoV2- | Ab | SARS-CoV2 | SARS- | | SARS-CoV1, | S; NTD | B-cells; SARS-CoV2 | 374 | QVQLVESGGGVVQPGRSLRLSCTASGFT | 1453 |
| 2307 | | | CoV1 | | SARS-CoV2 | | Human Patient | | FSSYAMHWVRQAPGKGLEWVALISYD | |
| | | | | | | | | | GNNKYYADSAKGRFTISRDNSKNTLYLQ | |
| | | | | | | | | | MNSLRSEDTAVYYCARDLGRGLDPWG | |
| | | | | | | | | | QGTLVTVSS | |
| CoV2- | Ab | SARS-CoV2 | SARS- | SARS-CoV2 | | S; RBD | B-cells; SARS-CoV2 | 375 | EVQLLESGGGLIQPGGSLRLSCAASGFTF | 1454 |
| 2308 | | | CoV1 | | | | Human Patient | | SNYAMSWVRQAPGKGLEWVSGIISSSG | |
| | | | | | | | | | GATYNADSVRGRFTTSRDNSKNILYLQM | |
| | | | | | | | | | NSLRGEDTAVYYCVKGLFDWFPLWGQ | |
| | | | | | | | | | GTMVTVSS | |
| CoV2- | Ab | SARS-CoV1, | | | SARS-CoV2 | S; non- | B-cells; SARS-CoV2 | 376 | QVQLQESGPGLVKPSQTLSLTCAVSGAS | 1455 |
| 2310 | | SARS-CoV2 | | | | RBC | Human Patient | | ISSGSYYWSWIRQPAGKGLEWIGRIYTS | |
| | | | | | | | | | GNTNYNPSLKSRVTISVDTSKNQFSLKLS | |
| | | | | | | | | | SVTAADTAVYYCATGYIGTYYYYMDVW | |
| | | | | | | | | | GKGTTVTVSS | |
| CoV2- | Ab | SARS-CoV1, | | | SARS-CoV2 | S; RBD | B-cells; SARS-CoV2 | 377 | EVQLVESGGGLVQPGRSLRLSCAASGFT | 1456 |
| 2313 | | SARS-CoV2 | | | | | Human Patient | | FDDYAMHWVRQAPGKGLEWVSGISW | |
| | | | | | | | | | NSGSIGYADSVKGRFTISRDNAKNSLYLQ | |
| | | | | | | | | | MNSLRAEDTALYYCAKVSSITSLLGYYFD | |
| | | | | | | | | | SWGQGTLVTVSS | |
| CoV2- | Ab | SARS-CoV2 | SARS- | | SARS-CoV1, | S; RBD | B-cells; SARS-CoV2 | 378 | QVTLRESGPALVKPTQTLSLTCTFSGFSL | 1457 |
| 2318 | | | CoV1 | | SARS-CoV2 | | Human Patient | | GTSGMCVSWIRQPPGKALEWLARIDW | |
| | | | | | | | | | DDDKYYSTSLKTRLTISKDTSKNQVVLTM | |
| | | | | | | | | | TNMDPVDTATYYCARGVVTYDYWGQG | |
| | | | | | | | | | TLVTVSS | |
| CoV2- | Ab | SARS-CoV1, | | | SARS-CoV2 | S; non- | B-cells; SARS-CoV2 | 379 | QVQLVESGGGVVQPGRSLRLSCAASGF | 1458 |
| 2322 | | SARS-CoV2 | | | | RBC | Human Patient | | TFSNYAMHWVRQAPGKGLDWVAVISY | |
| | | | | | | | | | DGSNRYYAASVKGRFTISRDNSKNTLYL | |
| | | | | | | | | | QMNSLRTEDTAVYFCARGDGYRSQFDP | |
| | | | | | | | | | WGQGTLVTVSS | |
| CoV2- | Ab | SARS-CoV1, | | | SARS-CoV2 | S; non- | B-cells; SARS-CoV2 | 380 | QITFKESGPTLVKPTETLTLTCTFSGFSVS | 1459 |
| 2325 | | SARS-CoV2 | | | | RBC | Human Patient | | TSGEGVGWIRQPPGKALEWLAVIYWD | |
| | | | | | | | | | DDKRYSPSLKSRLTITRDTSKNQVVLTMT | |
| | | | | | | | | | NMDPVDTATYYCAHRLWFRDAFDIWG | |
| | | | | | | | | | QGTTVTVSS | |
| CoV2- | Ab | SARS-CoV1, | | | SARS-CoV2 | S; RBD | B-cells; SARS-CoV2 | 381 | EVQLLESGGGLVQPGGSLRLSCAASGFT | 1460 |
| 2329 | | SARS-CoV2 | | | | | Human Patient | | FNNYAMSWVRQAPGKGLEWVSAIGGS | |
| | | | | | | | | | GGSTYYADSVKGRFTVSRDNSENTLYLQ | |
| | | | | | | | | | MSSLRAEDTAVYYCARVEGDWLLGGPY | |
| | | | | | | | | | YHYYGMDVWGQGTTVTVSS | |
| CoV2- | Ab | SARS-CoV1, | | | SARS-CoV2 | S; RBD | B-cells; SARS-CoV2 | 382 | QLQLQESGPGLVKPSETLSLTCTVSGGSI | 146: |
| 2331 | | SARS-CoV2 | | | | | Human Patient | | SSSSYYWGWIRQPPGNGLEWIGSIYYSG | |
| | | | | | | | | | STYYNPSLKGRVSISVDTSKNQFSLKLSSV | |
| | | | | | | | | | TAADTAVYYCARILVIFTLNWFDPWGQ | |
| | | | | | | | | | GTLVTVSS | |
| CoV2- | Ab | SARS-CoV1, | | | SARS-CoV2 | S; non- | B-cells; SARS-CoV2 | 383 | EVQLVESGGGLVQPGGSLRLSCAASGFT | 1462 |
| 2333 | | SARS-CoV2 | | | | RBD | Human Patient | | FSIYWMSWVRQAPGKGLQWVANIKQ | |
| | | | | | | | | | DASEKYYVDSVKGRFTISRDNAKNSLYLQ | |
| | | | | | | | | | MNSLRAEDTAVYYCARLGGSSWHFDY | |
| | | | | | | | | | WGQGTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | DIQMTQSPSSLSACVGDRVTITCRASQ | IGHV3-30- | IGHJ4 | IGKV1-39 | IGKJ1 | 2458 | ARDIDSGYDP | 3660 | QQSYSTP | Seth Zost et al., 2020 |
| 2299 | SISSYLNWYQQKPGKGPKLLIYAASSLQ | 3 | (Human) | (Human) | (Human) | | TPVFDY | | WT | (https://www.nature.co |
| | SGVPSRFSGSGSGTDFTLTISSLQPEDF | (Human) | | | | | | | | m/articles/s41591-020- |
| | ATYYCQQSYSTPWTFGQGTKVEIK | | | | | | | | | 0998-x) |
| CoV2- | DIQMTQSPSSVSASVGDRVTITCRASQ | IGHV4-4 | IGHJ4 | IGKV1-12 | IGKJ4 | 2459 | ASRWGDYFD | 3661 | QQANSLPL | Seth Zost et al., 2020 |
| 2300 | GISSWLAWYQQKPGKAPKLLIYAASSL | (Human) | (Human) | (Human) | (Human) | | SSGAYDS | | T | (https://www.nature.co |
| | QRGVPSRFSGSGSGTDFTLTISSLQPE | | | | | | | | | m/articles/s41591-020- |
| | DFATYYCQQANSLPLTFGGGTKVEVK | | | | | | | | | 0998-x) |
| CoV2- | QSVLTQPPSASGTPGQRVTISCSGSSS | IGHV3-21 | IGHJ4 | IGLV1-44 | IGLJ2 | 2460 | ARDPVWVDG | 3662 | AAWDDSL | Seth Zost et al., 2020 |
| 2304 | NIESNSVNWYQQLPGTAPKLLIYSNN | (Human) | (Human) | (Human) | (Human) | | ELLSGGIPFDY | | NGVV | (https://www.nature.co |
| | QRPSGVPDRFSGSKSGTSASLAISGLQ | | | | | | | | | m/articles/s41591-020- |
| | SEDEADYYCAAWDDSLNGVVFGGGT | | | | | | | | | 0998-x) |
| | KLTVL | | | | | | | | | |
| CoV2- | QSVLTQPPSASGTPGQRVTISCSGSTS | IGHV4-39 | IGHJ6 | IGLV1-44 | IGLJ3 | 2461 | AILWRGSSW | 3663 | AAWDDSL | Seth Zost et al., 2020 |
| 2305 | NIGSNTVNWYQQLPGTAPKLLIYSNN | (Human) | (Human) | (Human) | (Human) | | ADRHYYYYSM | | NGWV | (https://www.nature.co |
| | QRPSGVPDRFSGSKSGPSASLAISGLQ | | | | | | DV | | | m/articles/s41591-020- |
| | SVDEADYYCAAWDDSLNGWVFGGG | | | | | | | | | 0998-x) |
| | TKLTVL | | | | | | | | | |
| CoV2- | SYELTQSPSVSVSPGQTASITCSGDKLG | IGHV3-30- | IGHJ5 | IGLV3-1 | IGLJ3 | 2462 | ARDLGRGLDP | 3664 | QAWDSST | Seth Zost et al., 2020 |
| 2307 | DKYACWYQQRPGQSPVLVIYQDSQR | 3 (Human) | (Human) | (Human) | (Human) | | | | AV | (https://www.nature.co |
| | PSGIPERFSGSNSGNTATLTISGTQAM | | | | | | | | | m/articles/s41591-020- |
| | DEADYYCQAWDSSTAVFGGGTKLTVL | | | | | | | | | 0998-x) |
| CoV2- | DIVMTQSPATLSVSPGERAILSCRASQ | IGHV3-23 | IGHJ3 | IGKV3-15 | IGKJ1 | 2463 | VKGLFDWFPL | 3665 | HQYNNWP | Seth Zost et al., 2020 |
| 2308 | SVRSNLAWYQQKPGQAPRLLISGAST | (Human) | (Human) | (Human) | (Human) | | | | QT | (https://www.nature.co |
| | RATAIPARFSGSGSGTEFTLTITSLQSED | | | | | | | | | m/articles/s41591-020- |
| | CAVYYCHQYNNWPQTFGQGTKVEIK | | | | | | | | | 0998-x) |
| CoV2- | DIQMTQSPSSLSASVGDRVTITCRASQ | IGHV4-61 | IGHJ6 | IGKV1-39 | IGKJ4 | 2464 | ATGYIGTYYY | 3666 | QQSYSTLT | Seth Zost et al., 2020 |
| 2310 | SISTYLNWYQQKPGKAPKLLIYAASSLQ | (Human) | (Human) | (Human) | (Human) | | YMDV | | | (https://www.nature.co |
| | SGVPSRFSGSGSGTDFTLTISSLQPEDF | | | | | | | | | m/articles/s41591-020- |
| | ATYHCQQSYSTLTFGGGTKVEIK | | | | | | | | | 0998-x) |
| CoV2- | EIVLTQSPATLSLSPGERATLSCRASQS | IGHV3-9 | IGHJ4 | IGKV3-11 | IGKJ4 | 2465 | AKVSSITSLL | 3667 | QHRSNWP | Seth Zost et al., 2020 |
| 2313 | VSSYLAWYQQKPGQAPRLLIYDASNR | (Human) | (Human) | (Human) | (Human) | | GYYFDS | | PRLT | (https://www.nature.co |
| | ATGIPARFSGSGSGTDFTLTISSLEPEDF | | | | | | | | | m/articles/s41591-020- |
| | AVYYCQHRSNWPPRLTFGGGTKVEIK | | | | | | | | | 0998-x) |
| CoV2- | DIQMTQSPSSLSASVGDRVTITCRASQ | IGHV2-70 | IGHJ4 | IGKV1-39 | IGKJ5 | 2466 | ARGVVTYDY | 3668 | QQSYSTPG | Seth Zost et al., 2020 |
| 2318 | SIAGYLNWYQQKPGKAPKLLIYGTTSL | (Human) | (Human) | (Human) | (Human) | | | | T | (https://www.nature.co |
| | QSGVPVRFSGSGSGTDFTLTISSLQPE | | | | | | | | | m/articles/s41591-020- |
| | DFATYYCQQSYSTPGTFGQGTRLEIK | | | | | | | | | 0998-x) |
| CoV2- | EIVLTQSPGTLSLSPGERATLSCRASQS | IGHV3-30 | IGHJ5 | IGKV3-20 | IGKJ2 | 2467 | ARGDGYRSQ | 3669 | QQYGSSYT | Seth Zost et al., 2020 |
| 2322 | VSSSYLAWYQQKPGQAPRLLIYGASSR | (Human) | (Human) | (Human) | (Human) | | FDP | | | (https://www.nature.co |
| | ATGIPDRFSGSGSGTDFTLTISRLEPED | | | | | | | | | m/articles/s41591-020- |
| | FAVYYCQQYGSSYTFGQGTKLEIK | | | | | | | | | 0998-x) |
| CoV2- | DIQMTQSPSSLSASVGDRVTITCRARQ | IGHV2-5 | IGHJ3 | IGKV1-39 | IGKJ1 | 2468 | AHRLWFRDA | 3670 | QQTYSTF | Seth Zost et al., 2020 |
| 2325 | SISNYLNWYQQKPGKAPKLLIYAASSL | (Human) | (Human) | (Human) | (Human) | | FDI | | WT | (https://www.nature.co |
| | HSGVPSRFSGSGSGTDFTLTISSLQPED | | | | | | | | | m/articles/s41591-020- |
| | FATYYCQQTYSTFWTFGQGTNVEIK | | | | | | | | | 0998-x) |
| CoV2- | QSVLTQPPSASGTPGQRVTISCSGSSS | IGHV3-23 | IGHJ6 | IGLV1-47 | IGLJ3 | 2469 | ARVEGDWLL | 3671 | AAWDDSL | Seth Zost et al., 2020 |
| 2329 | NIGSNYVYWYQQLPGTAPKLLISRNN | (Human) | (Human) | (Human) | (Human) | | GGPYYHYYG | | SSWV | (https://www.nature.co |
| | QRPSGVPDRFSGSKSGTSASLAISGLRS | | | | | | MDV | | | m/articles/s41591-020- |
| | EDEADYYCAAWDDSLSSWVFGGGTK | | | | | | | | | 0998-x) |
| | LTVL | | | | | | | | | |
| CoV2- | NFMLTQPHSVSESPGKTVTISCTGSSG | IGHV4-39 | IGHJ5 | IGLV6-57 | IGLJ2 | 2470 | ARILVIFTLN | 3672 | QSYDSGNP | Seth Zost et al., 2020 |
| 233: | SIASNYVQWYQQRPGSAPTTVIYEDN | (Human) | (Human) | (Human) | (Human) | | WFDP | | | (https://www.nature.co |
| | QRPSGVPDRFSGSIDSSSNSASLTISGL | | | | | | | | | m/articles/s41591-020- |
| | KTEDEADYYCQSYDSGNPIFGGGTKLT | | | | | | | | | 0998-x) |
| | VL | | | | | | | | | |
| CoV2- | SYELTQPPSVSVSPGQTASITCSGDKLG | IGHV3-7 | IGHJ4 | IGLV3-1 | IGLJ3 | 2471 | ARLGGSSWH | 3673 | QAWSSST | Seth Zost et al., 2020 |
| 2333 | DKYSSWYQQKPGQSPVLVIYEDTKRPS | (Human) | (Human) | (Human) | (Human) | | FDY | | AV | (https://www.nature.co |
| | GIPERFSGSNSGNTATLTISGTQAMDE | | | | | | | | | m/articles/s41591-020- |
| | ADYYCQAWSSSTAVFGGGTKLTVL | | | | | | | | | 0998-x) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV2 | SARS- | | SARS-CoV1, | S; NTD | B-cells; SARS-CoV2 | 384 | QLQLQESGPGLVKPSETLSLTCTVSGGPI | 1463 |
| 2335 | | | CoV1 | | SARS-CoV2 | | Human Patient | | SSSRYYWGWIRQPPGKGLEWIGSIYYSG | |
| | | | | | | | | | STYYNPSLKSRVTISVDTSKNQFSLKLNSV | |
| | | | | | | | | | TAADTAVYYCARHDGSGEMDTITWGPI | |
| | | | | | | | | | YYYMDVWGKGTTVTVSS | |
| CoV2- | Ab | SARS-CoV1, | | | SARS-CoV2 | S; non- | B-cells; SARS-CoV2 | 385 | QITFKESGPTLVKPTETLTLTCTFSGFSVS | 1464 |
| 2337 | | SARS-CoV2 | | | | RBD | Human Patient | | TSGEGVGWIRQPPGKALEWLAVIYWD | |
| | | | | | | | | | DDKRYSPSLKSRLTITRDTSKNQVVLTMT | |
| | | | | | | | | | NMDPVDTATYYCAHRLWFRDAFDIWG | |
| | | | | | | | | | QGTTVTVSS | |
| CoV2- | Ab | SARS-CoV1, | | | SARS-CoV2 | S; non- | B-cells; SARS-CoV2 | 386 | QVQLVQSGAEVKKPGASVKVSCKASGY | 1465 |
| 2340 | | SARS-CoV2 | | | | RBD | Human Patient | | TFTSYGMHWVRQAPGQRLEWMGWIN | |
| | | | | | | | | | VGNGNTKYSQRFQGRVTITRDTSASTAY | |
| | | | | | | | | | MELSSLRSEDTAVYYCAMGPSAFSWLD | |
| | | | | | | | | | PWGQGTLVTVSS | |
| CoV2- | Ab | SARS-CoV1, | | | SARS-CoV2 | S; non- | B-cells; SARS-CoV2 | 387 | QVQLAESGGGVVQPGRSLRLSCAASGF | 1466 |
| 2341 | | SARS-CoV2 | | | | RBD | Human Patient | | TFSSYAMHWVRQAPGKGLEWVAVISY | |
| | | | | | | | | | DGSNKYYADSVKGRFTISRDNSKNTLYL | |
| | | | | | | | | | QMNSLRAEDTAVYYCARSTSGSYYYGM | |
| | | | | | | | | | DVWGQGTTVTVSS | |
| CoV2- | Ab | SARS-CoV1, | | | SARS-CoV2 | S; RBD | B-cells; SARS-CoV2 | 388 | QITLKESGPTLVKPTQTLTLTCTFSGFSLN | 1467 |
| 2342 | | SARS-CoV2 | | | | | Human Patient | | TSGVGVGWIRQPPGKALEWLALIYWD | |
| | | | | | | | | | DDKRYNPSLKSRLTITKDTSKNQVVLTM | |
| | | | | | | | | | TNMDPVDTATYYCAHRPPSYHGWCYF | |
| | | | | | | | | | DYWGQGNLVTVSS | |
| CoV2- | Ab | SARS-CoV1, | | | SARS-CoV2 | S; non- | B-cells; SARS-CoV2 | 389 | QVQVVQSGAEVKKPGASVKVSCKASGY | 1468 |
| 2343 | | SARS-CoV2 | | | | RBD | Human Patient | | TFKNYGISWVRQAPGQGLEWMGWISA | |
| | | | | | | | | | YTGNTNYAQKFQGRMTMTTDTSTGTG | |
| | | | | | | | | | YMELRSLRSDDTAVYYCARVQRRRLDY | |
| | | | | | | | | | WGQGTLVIVSS | |
| CoV2- | Ab | SARS-CoV2 | SARS- | | SARS-CoV1, | S; NTD | B-cells; SARS-CoV2 | 390 | QVQLQQWGAGLLKPSETLSLTCAVYGG | 1469 |
| 2346 | | | CoV1 | | SARS-CoV2 | | Human Patient | | SFSGHYWSWIRQPPGKGLEWIGEINHS | |
| | | | | | | | | | GSTNYNPSLKSRVTISVDTSKNQFSLKLSS | |
| | | | | | | | | | VTAADTAVYYCARPPQAARIHYYYYMD | |
| | | | | | | | | | VWGKGTTVTVSS | |
| CoV2- | Ab | SARS-CoV2 | SARS- | | SARS-CoV1, | S; RBD | B-cells; SARS-CoV2 | 391 | EVQLVQSGAEVKKPGESLKISCKGSGYSF | 1470 |
| 2351 | | | CoV1 | | SARS-CoV2 | | Human Patient | | TSYWIGWVRQMPGKGLEWMGIIYPGD | |
| | | | | | | | | | SDTTYNPSFQGQVTISADKSLTTAFLHW | |
| | | | | | | | | | SSLKASDTAIYYCARRFYGPSSFDYWGQ | |
| | | | | | | | | | GTLVIVSS | |
| CoV2- | Ab | SARS-CoV2 | SARS- | | SARS-CoV1, | S; RBD | B-cells; SARS-CoV2 | 392 | QVQLVQSGAEVKRPGASMNISCKASGY | 1471 |
| 2352 | | | CoV1 | | SARS-CoV2 | | Human Patient | | NFNNNYIYWVRQAPGQGLEWMGVVN | |
| | | | | | | | | | PTGGGTAYAQGFQDRVTITSDTPRNTVY | |
| | | | | | | | | | LGVTGLHSEDTAVYFCARGGEWRIVPG | |
| | | | | | | | | | GRDYFDYWGQGTLVTVSA | |
| CoV2- | Ab | SARS-CoV2 | SARS- | SARS-CoV2 | | S; RBD | B-cells; SARS-CoV2 | 393 | EVQLVQSGAEVKKPGESLKISCKGFGYN | 1472 |
| 2353 | | | CoV1 | | | | Human Patient | | FTNYWIGWVRQMPGKGLEWMGIIYPG | |
| | | | | | | | | | DSETRNSPSFQGQVTISADKSMSTAYLQ | |
| | | | | | | | | | WSSLKASDTAMYYCARLGVSKYCSGGR | |
| | | | | | | | | | CLSGGSNWFDPWGQGTLVTVSS | |
| CoV2- | Ab | SARS-CoV1, | | SARS-CoV2 | | S; RBD | B-cells; SARS-CoV2 | 394 | EVHLVGSGGGLIQPGGSLRLSCAASGFT | 1473 |
| 2354 | | SARS-CoV2 | | | | | Human Patient | | VSSNFMSWVRQAPGKGLEWVSIIHNG | |
| | | | | | | | | | GDSYYTDSVKGRFTISRDNSKNTLYLQM | |
| | | | | | | | | | NNLRAEDTAVYYCASSSWLRGAFDIWG | |
| | | | | | | | | | QGTMVTVSS | |
| CoV2- | Ab | SARS-CoV2 | SARS- | SARS-CoV2 | | S; RBD | B-cells; SARS-CoV2 | 395 | QMQLVQSGPEVKKPGTSVKVSCKTSGF | 1474 |
| 2355 | | | CoV1 | | | | Human Patient | | TFTSSAIQWVRQARGQRLEWIGWIVVG | |
| | | | | | | | | | SGNTNYAQKFQERVTITRDMSTSTAYM | |
| | | | | | | | | | ELSSLRSEDTAVYYCAAPHCNRTSCYDAF | |
| | | | | | | | | | DLWGQGTMVTVSS | |
| CoV2- | Ab | SARS-CoV1, | | | SARS-CoV2 | S; RBD | B-cells; SARS-CoV2 | 396 | EVQLVESGGDLVKPGRSLRLSCSASGFTF | 1475 |
| 2357 | | SARS-CoV2 | | | | | Human Patient | | GDYTMSWFRQAPGKGLEWVAFIRSKAY | |
| | | | | | | | | | GGTTEYAASVIGRFTISRDDSKSIAYLQM | |
| | | | | | | | | | NSLKSEDTAVYYCSRVRGSFYGSVGKNY | |
| | | | | | | | | | GMDVWGQGTTVTVSS | |
| CoV2- | Ab | SARS-CoV1, | | | SARS-CoV2 | S; RBD | B-cells; SARS-CoV2 | 397 | QVQLVESGGGVVQPGRSLRLSCAASGF | 1476 |
| 2358 | | SARS-CoV2 | | | | | Human Patient | | SFSSYVMNWVRQAPGKGLEWVAVISY | |
| | | | | | | | | | DGSSKYYADSVKGRFTISRDNSKNTLYLQ | |
| | | | | | | | | | MNSLRAEDTAVYYCARDIDSGYDPTPVF | |
| | | | | | | | | | DYWGQGTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | QSALTQPASVSGSPGQSITISCTGTSSD | IGHV4-39 | IGHJ6 | IGLV2-14 | IGLJ2 | 2472 | ARHDGSGEM | 3674 | SSYTSSTLN | Seth Zost et al., 2020 |
| 2335 | VGGYNYVSWYQQHPGKAPKLMIYYV | (Human) | (Human) | (Human) | (Human) | | DTITWGPIYYY | | VL | (https://www.nature.co |
| | SNRPSGVSNRFSGSKSGNTASLTISGL | | | | | | MDV | | | m/articles/s41591-020- |
| | QAEDEADYYCSSYTSSTLNVLFGGGTK | | | | | | | | | 0998-x) |
| | LTVL | | | | | | | | | |
| CoV2- | DIQMTQSPSSLSASVGDRVTITCRARQ | IGHV2-5 | IGHJ3 | IGKV1-39 | IGKJ5 | 2473 | AHRLWFRDA | 3675 | QQSYSTPG | Seth Zost et al., 2020 |
| 2337 | SISNYLNWYQQKPGKAPKLLIYAASSL | (Human) | (Human) | (Human) | (Human) | | FDI | | T | (https://www.nature.co |
| | HSGVPSRFSGSGSGTDFTLTISSLQPED | | | | | | | | | m/articles/s41591-020- |
| | FATYYCQQSYSTPGTFGQGTRLEIK | | | | | | | | | 0998-x |
| CoV2- | QSVLTQPPSVSGAPGQRVTISCTGSSS | IGHV1-3 | IGHJ5 | IGLV1-40 | IGLJ1 | 2474 | AMGPSAFSW | 3676 | QSYDSSLS | Seth Zost et al., 2020 |
| 2340 | NIGAGYDVHWYQHLPGTAPKLLIYGN | (Human) | (Human) | (Human) | (Human) | | LDP | | GWSV | (https://www.nature.co |
| | SNRPSGVPDRFSGSKSGTSASLAITGL | | | | | | | | | m/articles/s41591-020- |
| | QAEDEADYYCQSYDSSLSGWSVFGTG | | | | | | | | | 0998-x) |
| | TKVTVL | | | | | | | | | |
| CoV2- | QSALTQPASVSGSPGQSITISCTGTSSD | IGHV3-30- | IGHJ6 | IGLV2-14 | IGLJ1 | 2475 | ARSTSGSYYY | 3677 | SSYTSSSTL | Seth Zost et al., 2020 |
| 2341 | VGDYNYVSWYQQHPGKAPKLMIYDV | 3 | (Human) | (Human) | (Human) | | GMDV | | LYV | (https://www.nature.co |
| | SNRPSGVSNRFSGSKSGNTASLTISGL | (Human) | | | | | | | | m/articles/s41591-020- |
| | QAEDEAEYYCSSYTSSSTLLYVFGTGTK | | | | | | | | | 0998-x) |
| | VTVL | | | | | | | | | |
| CoV2- | DIQMTQSPSSLSASVGDRVTITCRASQ | IGHV2-5 | IGHJ4 | IGKV1-39 | IGKJ2 | 2476 | AHRPPSYHG | 3678 | QQSYSTH | Seth Zost et al., 2020 |
| 2342 | SISIYLNWYQQKPGKAPKLLIYAASTLQ | (Human) | (Human) | (Human) | (Human) | | WCYFDY | | MST | (https://www.nature.co |
| | SGVPSRFSGSGSGTDFTLTISSLQPEDF | | | | | | | | | m/articles/s41591-020- |
| | ATYYCQQSYSTHMSTFGQGTKLEIK | | | | | | | | | 0998-x) |
| CoV2- | DIVVTQTPLSLSVTPGQPASISCKSSET | IGHV1-18 | IGHJ4 | IGKV2D-29 | IGKJ5 | 2477 | ARVQRRRLDY | 3679 | MQSIQLA | Seth Zost et al., 2020 |
| 2343 | LLHSDGKTYLSWYLQKPGQPPQLLIYEV | (Human) | (Human) | (Human) | (Human) | | | | | (https://www.nature.co |
| | SNRFSGVPDRFSGSGSGTDFTLKIGRV | | | | | | | | | m/articles/s41591-020- |
| | EAEDVGLYYCMQSIQLAFGQGTRLEIE | | | | | | | | | 0998-x) |
| CoV2- | EIVMTQSPATLSVSPGERATLSCRASQ | IGHV4-34 | IGHJ6 | IGKV3-15 | IGKJ4 | 2478 | ARPPQAARIH | 3680 | QQYNYWP | Seth Zost et al., 2020 |
| 2346 | SVSSNLAWYQQKPGQAPRLLIYGAST | (Human) | (Human) | (Human) | (Human) | | YYYYMDV | | PLT | (https://www.nature.co |
| | RATGIPARFSGSGSGTEFTLTISSLQSE | | | | | | | | | m/articles/s41591-020- |
| | DFAVYYCQQYNYWPPLTFGGGTKVEI | | | | | | | | | 0998-x) |
| | K | | | | | | | | | |
| CoV2- | SSELTQDPAVSVALGQTVTITCQGDSL | IGHV5-51 | IGHJ4 | IGLV3-19 | IGLJ2 | 2479 | ARRFYGPSSF | 3681 | SSGDSSTD | Seth Zost et al., 2020 |
| 2351 | RSHYASWYKVKPGQAPLLVIYGRNNR | (Human) | (Human) | (Human) | (Human) | | DY | | HHVV | (https://www.nature.co |
| | PSGIPDRFSGSRSGNTASLTITGAQVD | | | | | | | | | m/articles/s41591-020- |
| | DEADYYCSSGDSSTDHHVVFAGGTKL | | | | | | | | | 0998-x) |
| | AVV | | | | | | | | | |
| CoV2- | DMEVTQFPSSLSASIGDRVTITCRATQ | IGHV1-46 | IGHJ4 | IGKV1-39 | IGKJ4 | 2480 | ARGGEWRIV | 3682 | HQSYGVPI | Seth Zost et al., 2020 |
| 2352 | SISTYLNWYQQKPGKAPNLLIYSASHL | (Human) | (Human) | (Human) | (Human) | | PGGRDYFDY | | T | (https://www.nature.co |
| | QRGVPARFSGSGSGTEFTLTITTLQPE | | | | | | | | | m/articles/s41591-020- |
| | DVGTYYCHQSYGVPITFGGGTKVDIK | | | | | | | | | 0998-x) |
| CoV2- | EIVMTQSPATLSVSPGERATLSCRASQ | IGHV5-51 | IGHJ5 | IGKV3-15 | IGKJ2 | 2481 | ARLGVSKYCS | 3683 | QQYNNWP | Seth Zost et al., 2020 |
| 2353 | SVSSNLAWYQQKPGQAPRLLIYGAST | (Human) | (Human) | (Human) | (Human) | | GGRCLSGGS | | PMYT | (https://www.nature.co |
| | RATGIPARFSGSGSGTEFTLTISSLQSE | | | | | | NWFDP | | | m/articles/s41591-020- |
| | DFAVYYCQQYNNWPPMYTFGQGTKL | | | | | | | | | 0998-x) |
| | EIK | | | | | | | | | |
| CoV2- | NFMLTQPHSVSESPGKTVTISCTGSSG | IGHV3-53 | IGHJ3 | IGLV6-57 | IGLJ2 | 2482 | ASSSWLRGAF | 3684 | QSYDSSKY | Seth Zost et al., 2020 |
| 2354 | SIASNYVQWYQQRPGSAPTTVIYEDN | (Human) | (Human) | (Human) | (Human) | | DI | | VV | (https://www.nature.co |
| | QRPSGVPDRFSGSIDSSSNSASLTISGL | | | | | | | | | m/articles/s41591-020- |
| | KTEDEADYYCQSYDSSKYVVFGGGTKL | | | | | | | | | 0998-x) |
| | TVL | | | | | | | | | |
| CoV2- | EIVLTQSPGTLSLSPGERATLSCRASQS | IGHV1-58 | IGHJ3 | IGKV3-20 | IGKJ1 | 2483 | AAPHCNRTSC | 3685 | QQYGSSP | Seth Zost et al., 2020 |
| 2355 | VSSSYLGWYQQKPGQAPRLLIYGASSR | (Human) | (Human) | (Human) | (Human) | | YDAFDL | | WT | (https://www.nature.co |
| | ATGIPDRFSGSGSGTDFTLTISRLEPED | | | | | | | | | m/articles/s41591-020- |
| | FAVYYCQQYGSSPWTFGQGTKVEIK | | | | | | | | | 0998-x) |
| CoV2- | DIVMTQSPLSLPVTPGEPASISCRSSQS | IGHV3-49 | IGHJ6 | IGKV2-28 | IGKJ2 | 2484 | SRVRGSFYGS | 3686 | MQALQTP | Seth Zost et al., 2020 |
| 2357 | LLHSNGYNYLDWYLQKPGQSPQLLIYL | (Human) | (Human) | (Human) | (Human) | | VGKNYGMDV | | LYT | (https://www.nature.co |
| | GSTRASGVPDRFSGSGSGTDFTLKISR | | | | | | | | | m/articles/s41591-020- |
| | VEAEDVGVYYCMQALQTPLYTFGQGT | | | | | | | | | 0998-x) |
| | KLEIK | | | | | | | | | |
| CoV2- | DIQMTQSPSSLSACVGDRVTITCRASQ | IGHV3-30- | IGHJ4 | IGKV1-39 | IGKJ5 | 2485 | ARDIDSGYDP | 3687 | QQSYSSLSI | Seth Zost et al., 2020 |
| 2358 | SISSYLNWYQQKPGKGPKLLIYAASSLQ | 3 | (Human) | (Human) | (Human) | | TPVFDY | | T | (https://www.nature.co |
| | SGVPSRFSGSGSGTDFTLTISSLQPEDF | (Human) | | | | | | | | m/articles/s41591-020- |
| | ATYYCQQSYSSLSITFGQGTRLEIK | | | | | | | | | 0998-x) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV1, | | | SARS-CoV2 | S; non- | B-cells; SARS-CoV2 | 398 | QVQLVQSGAEVKKPGSSVKVSCKASGG | 1477 |
| 2367 | | SARS-CoV2 | | | | RBD | Human Patient | | TFSSYAISWVRQAPGQGLEWMGGIIPIF | |
| | | | | | | | | | GAANYAQNFQGRVTITADESTSTGYMQ | |
| | | | | | | | | | LSSLRFEDTAVYYCARTSHYDSSGSYFEY | |
| | | | | | | | | | WGQGTLVTVSS | |
| CoV2- | Ab | SARS-CoV2 | SARS- | | SARS-CoV1, | S; non- | B-cells; SARS-CoV2 | 399 | EVQLVQSGAEVKKPGESLKISCKGSGYSF | 1478 |
| 2368 | | | CoV1 | | SARS-CoV2 | RBD | Human Patient | | TTYWIGWVRQMPGKGLEWMGIIYPGD | |
| | | | | | | | | | SDTRYSPSFQGQVTISADKSISTAYLQWS | |
| | | | | | | | | | SLKASDTAMYYCARRRGGIGIEYGMDV | |
| | | | | | | | | | WGQGTTVTVSS | |
| CoV2- | Ab | SARS-CoV2 | SARS- | | SARS-CoV1, | S; non- | B-cells; SARS-CoV2 | 400 | QVQLVESGGGVVQPGRSLRLSCAASGF | 1479 |
| 2369 | | | CoV1 | | SARS-CoV2 | RBD | Human Patient | | TFSSYAMHWVRQAPGKGLEWVADISY | |
| | | | | | | | | | DGSEKYYADSVKGRFTIYRDNSKNTLYLQ | |
| | | | | | | | | | MNSLRAEDTAVYYCAKDFGGDNTAMV | |
| | | | | | | | | | EYFFDFWGQGTLVTVSS | |
| CoV2- | Ab | SARS-CoV2 | SARS- | | SARS-CoV1, | S; RBD | B-cells; SARS-CoV2 | 401 | QVQLQESGPGLVKPSETLSLTCTVSGGS | 1480 |
| 2370 | | | CoV1 | | SARS-CoV2 | | Human Patient | | VSSGSYYWSWIRQPPGKGLECIGYIYYSG | |
| | | | | | | | | | SSNYNPSLKSRVTISVDTSKNQFSLKMSS | |
| | | | | | | | | | VTAADTAVYYCAGSPVPPTIVGASYWG | |
| | | | | | | | | | QGTLVTDSS | |
| CoV2- | Ab | SARS-CoV1, | | | SARS-CoV2 | S; non- | B-cells; SARS-CoV2 | 402 | QVQLVESGGGVVQPGRSLRLSCAASGF | 1481 |
| 2371 | | SARS-CoV2 | | | | RBD | Human Patient | | TFSNYGMHWVRQAPGKGLEWVAVISY | |
| | | | | | | | | | DGTNKYYADSVKGRFTISRDNSKNTLYL | |
| | | | | | | | | | QMNSLRADDTAVYYCAKGRGNYLTFFD | |
| | | | | | | | | | SWGQGTLVTVSS | |
| CoV2- | Ab | SARS-CoV2 | SARS- | | SARS-CoV1, | S; non- | B-cells; SARS-CoV2 | 403 | EVQLVESGGGLVQPGGSLRLSCAASGLT | 1482 |
| 2373 | | | CoV1 | | SARS-CoV2 | RBD | Human Patient | | VSSNYMSWVRQAPGKGLEWVSVIYSG | |
| | | | | | | | | | GSTYYADSVKGRFTISGDNSKNTLYLQM | |
| | | | | | | | | | NSLRVDDTAVYYCARDPGSRYSGGWYD | |
| | | | | | | | | | YYYAMDVWGQGTTVTVSS | |
| CoV2- | Ab | SARS-CoV2 | SARS- | | SARS-CoV1, | S; non- | B-cells; SARS-CoV2 | 404 | EVQLVQSGAEVKKPGESLKISCKGSGYSF | 1483 |
| 2378 | | | CoV1 | | SARS-CoV2 | RBD | Human Patient | | PTYWIGWVRQMPGKGLEWMGIIYPGD | |
| | | | | | | | | | SDTRYGPSFQGQVTISADKSISTAYLQWS | |
| | | | | | | | | | SLKASDTAMYYCARRDTDFDYWGQGTL | |
| | | | | | | | | | VTVSS | |
| CoV2- | Ab | SARS-CoV2 | SARS- | SARS-CoV2 | | S; RBD | B-cells; SARS-CoV2 | 405 | QMQLVQSGPEVKKPGTSVKVSCKASGF | 1484 |
| 2381 | | | CoV1 | | | | Human Patient | | TFTSSAVQWVRQARGQRLEWIGWIAV | |
| | | | | | | | | | GSGNTNYAQKFQERVSITRDMSTSTAY | |
| | | | | | | | | | MELSSLRSEDTAVYYCAAPYCSRTSCHD | |
| | | | | | | | | | AFDIWGQGTKVTVSS | |
| CoV2- | Ab | SARS-CoV1, | | | SARS-CoV2 | S; RBD | B-cells; SARS-CoV2 | 406 | QVQLVESGGGVVQPGRSLRLSCVASGF | 1485 |
| 2382 | | SARS-CoV2 | | | | | Human Patient | | TFSSYGMHWVRQAPGKGLEWVAVISF | |
| | | | | | | | | | DGSNKYYADSVKGRFTISRDNSKNTLYL | |
| | | | | | | | | | QMNSLRAEDTAVFYCAKDLPPYASGWY | |
| | | | | | | | | | EGGFDYWGRGTQVTVSS | |
| CoV2- | Ab | SARS-CoV2 | SARS- | | SARS-CoV1, | S; non- | B-cells; SARS-CoV2 | 407 | QVQLVESGGGVVQPGRSLRLSCAASGF | 1486 |
| 2383 | | | CoV1 | | SARS-CoV2 | RBD | Human Patient | | TFSSYGMHWVRQAPGKGLEWVAVVSY | |
| | | | | | | | | | DGSNKYYTDSVKGRFTISRDNSKNTLFLQ | |
| | | | | | | | | | MIGLREEDTAVYYCAQGRGGYYSPFDD | |
| | | | | | | | | | WGQGTLVTVSS | |
| CoV2- | Ab | SARS-CoV2 | SARS- | | SARS-CoV1, | S; NTD | B-cells; SARS-CoV2 | 408 | QVTLKESGPVLVKPTETLTLTCTVSGFSLS | 1487 |
| 2384 | | | CoV1 | | SARS-CoV2 | | Human Patient | | NARMGVSWIRQPPGKALEWLAHIFSG | |
| | | | | | | | | | DEKSYSTSLKSRLTISKDTSKSQVVLTMT | |
| | | | | | | | | | NMDPLDTATYYCARTTWGTWIQAWYF | |
| | | | | | | | | | DIWGRGTLVTVSS | |
| CoV2- | Ab | SARS-CoV2 | SARS- | | SARS-CoV1, | S; non- | B-cells; SARS-CoV2 | 409 | QVQLVESGGGVVQPGRSLRLSCAASGF | 1488 |
| 2386 | | | CoV1 | | SARS-CoV2 | RBD | Human Patient | | TFSSYGMHWVRQAPGKGLEWVAVIWF | |
| | | | | | | | | | DGSNKHYADSVKGRFTISRDNSKNTLYL | |
| | | | | | | | | | QMNSLRAEDTAVYYCAREGDFWSGYYT | |
| | | | | | | | | | GWFDPWGQGTLVTVSS | |
| CoV2- | Ab | SARS-CoV1, | | | SARS-CoV2 | S; non- | B-cells; SARS-CoV2 | 410 | QVQLVESGGGVVQPGRSLRLSCAASGF | 1489 |
| 2387 | | SARS-CoV2 | | | | RBD | Human Patient | | TFSSYGMHWVRQAPGKGLEWVAVISY | |
| | | | | | | | | | DGSNKYYADSVKGRFTISRDNSKNTLYL | |
| | | | | | | | | | QMNSLRAEDTAVYYCAKDLTIVVIPAAP | |
| | | | | | | | | | NFDYWGQGTLVTVSS | |
| CoV2- | Ab | SARS-CoV1, | | | SARS-CoV2 | S; non- | B-cells; SARS-CoV2 | 411 | EVQLLESGGGLVQPGGSLRLSCAASGFT | 1490 |
| 2388 | | SARS-CoV2 | | | | RBD | Human Patient | | FSSYAMSWVRQAPGKGLEWVSFISGTG | |
| | | | | | | | | | DSTYYADSVKGRFTISRDNSKNTLYLQM | |
| | | | | | | | | | NSLRAEDTAVYYCAKDQARVQDYIWGS | |
| | | | | | | | | | YRSYGMDVWGLGTTVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | EIVLTQSPATLSLSPGERATLSCRASQS | IGHV1-69 | IGHJ4 | IGKV3-11 | IGKJ4 | 2486 | ARTSHYDSSG | 3688 | HKRSNWP | Seth Zost et al., 2020 |
| 2367 | VSSYLAWYQQKPGQAPRLLIYDASNR | (Human) | (Human) | (Human) | (Human) | | SYFEY | | PSLT | (https://www.nature.co |
| | ATGIPARFSGSGSGTDFTLTISSLDPED | | | | | | | | | m/articles/s41591-020- |
| | FAVYYCHKRSNWPPSLTFGGGTKVEIK | | | | | | | | | 0998-x) |
| CoV2- | SNELTQPPSVSVSPGQTARITCSGDAL | IGHV5-51 | IGHJ6 | IGLV3-25 | IGLJ2 | 2487 | ARRRGGIGIE | 3689 | QSTASSGT | Seth Zost et al., 2020 |
| 2368 | PKQYAYWYQQKPGQAPVLVIYKDTER | (Human) | (Human) | (Human) | (Human) | | YGMDV | | VV | (https://www.nature.co |
| | PSGIPERFSGSSSGTTVTLTISGVQAED | | | | | | | | | m/articles/s41591-020- |
| | EADYYCQSTASSGTYVVFGGGTKLTVL | | | | | | | | | 0998-x) |
| CoV2- | DIQMTQSPSTLSASVGDRVTITCRASQ | IGHV3-30 | IGHJ4 | IGKV1-5 | IGKJ1 | 2488 | AKDFGGDNT | 3690 | QQYNSYSP | Seth Zost et al., 2020 |
| 2369 | SISSWLAWYQQKPGKAPKLLIYKASSL | (Human) | (Human) | (Human) | (Human) | | AMVEYFFDF | | T | (https://www.nature.co |
| | ESGVPSRFSGSGSGTEFTLTISSLQPDD | | | | | | | | | m/articles/s41591-020- |
| | FATYYCQQYNSYSPTFGQGTKVEIK | | | | | | | | | 0998-x) |
| CoV2- | NFMLTQPHSVSESPGKTVTFSCTGSSG | IGHV4-61 | IGHJ4 | IGLV6-57 | IGLJ2 | 2489 | AGSPVPPTIV | 3691 | QSYDGINR | Seth Zost et al., 2020 |
| 2370 | SIASNYVQWYQQRPGSAPTTVIYEDN | (Human) | (Human) | (Human) | (Human) | | GASY | | WLV | (https://www.nature.co |
| | QRPSGVPDRFSGSIDSSSNSASLTISGL | | | | | | | | | m/articles/s41591-020- |
| | KTEDEADYYCQSYDGINRWLVFGGGT | | | | | | | | | 0998-x) |
| | KLTVL | | | | | | | | | |
| CoV2- | EIVMTQSPATLSVSPGERATLSCRASQ | IGHV3-30 | IGHJ4 | IGKV3-15 | IGKJ1 | 2490 | AKGRGNYLTF | 3692 | QQYNNWP | Seth Zost et al., 2020 |
| 2371 | SVSSNLAWYQQKPGQAPRLLIYGAST | (Human) | (Human) | (Human) | (Human) | | FDS | | GT | (https://www.nature.co |
| | RATGIPARFSGSGSGTEFTLTISSLQSE | | | | | | | | | m/articles/s41591-020- |
| | DFAVYYCQQYNNWPGTFGQGTKVEI | | | | | | | | | 0998-x) |
| | K | | | | | | | | | |
| CoV2- | EIVLTQSPGTLSLSPGERATLSCRASQS | IGHV3-66 | IGHJ6 | IGKV3-20 | IGKJ2 | 2491 | ARDPGSRYSG | 3693 | QQYGSSPP | Seth Zost et al., 2020 |
| 2373 | VSSSYLAWYQQKPGQAPRLLIYGASSR | (Human) | (Human) | (Human) | (Human) | | GWYDYYYAM | | YT | (https://www.nature.co |
| | ATGIPDRFSGSGSGTDFTLTISRLEPED | | | | | | DV | | | m/articles/s41591-020- |
| | FAVYYCQQYGSSPPYTFGQGTKLEIK | | | | | | | | | 0998-x) |
| CoV2- | QPVLTQPPSASASLGASVTLTCTLSSGY | IGHV5-51 | IGHJ4 | IGLV9-49 | IGLJ2 | 2492 | ARRDTDFDY | 3694 | GADHGSG | Seth Zost et al., 2020 |
| 2378 | SNYKVDWFQQRPGKGPRFVMRVGT | (Human) | (Human) | (Human) | (Human) | | | | SNFVYVV | (https://www.nature.co |
| | GGIVGSKGDGIPDRFSVLGSGLNRYLTI | | | | | | | | | m/articles/s41591-020- |
| | KNIQEEDESDYHCGADHGSGSNFVYV | | | | | | | | | 0998-x) |
| | VFGGGTKLTVL | | | | | | | | | |
| CoV2- | EVVLTQSPGTLSLSPGERATLSCRASQS | IGHV1-58 | IGHJ3 | IGKV3-20 | IGKJ1 | 2493 | AAPYCSRTSC | 3695 | QHFGSSSQ | Seth Zost et al., 2020 |
| 2381 | VSSSYLAWYQQKPGQAPRLLIYGASSR | (Human) | (Human) | (Human) | (Human) | | HDAFDI | | WT | (https://www.nature.co |
| | ATGIPDRFSGSGSGTDFTLTISRLEPED | | | | | | | | | m/articles/s41591-020- |
| | FAVYHCQHFGSSSQWTFGQGTKVEIK | | | | | | | | | 0998-x) |
| CoV2- | QTVVTQEPSLTVSPGGTVTLTCASSTG | IGHV3-30 | IGHJ4 | IGLV7-43 | IGLJ3 | 2494 | AKDLPPYASG | 3696 | LLYYGGPW | Seth Zost et al., 2020 |
| 2382 | AVTSGYFPNWFQQKPGQAPRALIFST | (Human) | (Human) | (Human) | (Human) | | WYEGGFDY | | V | (https://www.nature.co |
| | NNRHSWTPARFSGSLLGDKAALTLSG | | | | | | | | | m/articles/s41591-020- |
| | VQPEDEAEYYCLLYYGGPWVFGGGTK | | | | | | | | | 0998-x) |
| | LTVL | | | | | | | | | |
| CoV2- | EIVMTQSPGTLSVSPGERATLSCRASQ | IGHV3-30 | IGHJ4 | IGKV3-15 | IGKJ1 | 2495 | AQGRGGYYS | 3697 | QQYNNWP | Seth Zost et al., 2020 |
| 2383 | TLSSNLAWYQQKPGQAPRLLIYGASTR | (Human) | (Human) | (Human) | (Human) | | PFDD | | LA | (https://www.nature.co |
| | ATGIPARFSGSGSGTEFTLTISSLQSEDF | | | | | | | | | m/articles/s41591-020- |
| | AVYHCQQYNNWPLAFGQGTKVEIK | | | | | | | | | 0998-x) |
| CoV2- | QSALTQPASVSGSPGQSITISCTGTSSD | IGHV2-26 | IGHJ2 | IGLV2-23 | IGLJ2 | 2496 | ARTTWGTWI | 3698 | CSYAGGNT | Seth Zost et al., 2020 |
| 2384 | IGSYNLVSWYQQYPGKAPKLMIYEVSK | (Human) | (Human) | (Human) | (Human) | | QAWYFDI | | FVV | (https://www.nature.co |
| | RPSGVSNRFSGSKSGNTASLTISGLQA | | | | | | | | | m/articles/s41591-020- |
| | EDEADYYCCSYAGGNTFVVFGGGTKL | | | | | | | | | 0998-x) |
| | TVL | | | | | | | | | |
| CoV2- | EIVMTQSPATLSVSPGERATLSCRASQ | IGHV3-33 | IGHJ5 | IGKV3-15 | IGKJ1 | 2497 | AREGDFWSG | 3699 | QQYNNWP | Seth Zost et al., 2020 |
| 2386 | SVSSNLAWYQQKPGQAPRLLIYGAST | (Human) | (Human) | (Human) | (Human) | | YYTGWFDP | | RT | (https://www.nature.co |
| | RATGIPARFSGSGSGTEFTLTISSLQSE | | | | | | | | | m/articles/s41591-020- |
| | DFAVYYCQQYNNWPRTFGQGNRVEI | | | | | | | | | 0998-x) |
| | N | | | | | | | | | |
| CoV2- | QSALTQPASVSGSPGQSITISCTGTSSD | IGHV3-30 | IGHJ4 | IGLV2-14 | IGLJ2 | 2498 | AKDLTIVVIPA | 3700 | SSYTSSST | Seth Zost et al., 2020 |
| 2387 | VGGYNYVSWYQQHPGKAPKLMIYDV | (Human) | (Human) | (Human) | (Human) | | APNFDY | | PVV | (https://www.nature.co |
| | SNRPSGVSNRFSGSRSGNTASLTISGL | | | | | | | | | m/articles/s41591-020- |
| | QAEDEADYYCSSYTSSSTPVVFGGGTK | | | | | | | | | 0998-x) |
| | LTVL | | | | | | | | | |
| CoV2- | DIVMTQSPLSLPVTPGEPASISCRSSQS | IGHV3-23 | IGHJ6 | IGKV2-28 | IGKJ1 | 2499 | AKDQARVQD | 3701 | MQALQTP | Seth Zost et al., 2020 |
| 2388 | LLHSNGYNYLDWYLQKPGQSPHLLIYL | (Human) | (Human) | (Human) | (Human) | | YIWGSYRSYG | | RT | (https://www.nature.co |
| | GSNRASGVPDRFSGSGSGTDFTLKISR | | | | | | MDV | | | m/articles/s41591-020- |
| | VEAEDVGVYYCMQALQTPRTFGQGT | | | | | | | | | 0998-x) |
| | KVEIK | | | | | | | | | |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV2 | SARS- | SARS-CoV2 | | S; RBD | B-cells; SARS-CoV2 | 412 | EVQLVESGGGLVQPGRSLRLSCAASGFT | 1491 |
| 2389 | | | CoV1 | | | | Human Patient | | FDDYAMHWVRQAPGKGLEWVSGISW | |
| | | | | | | | | | NSGTIGYADSVKGRFIISRDNAKNSLYLQ | |
| | | | | | | | | | MNSLRPEDTALYYCAKDIIRQGEDGMD | |
| | | | | | | | | | VWGQGTTVTVSS | |
| CoV2- | Ab | SARS-CoV2 | SARS- | SARS-CoV2 | | S; RBD | B-cells; SARS-CoV2 | 413 | QVQLVQSGAEVKKPGASVKVSCKASGY | 1492 |
| 2391 | | | CoV1 | | | | Human Patient | | TFGSFDINWVRQATGQGLEWMGRMN | |
| | | | | | | | | | SNSGNTAYAQKFQGRVTMTRDTSTNTA | |
| | | | | | | | | | YMELSSLRSEDTAMYYCARMRSGWPTH | |
| | | | | | | | | | GRPDDFWGRGTLVTVSS | |
| CoV2- | Ab | SARS-CoV2 | SARS- | | SARS-CoV1, | S; NTD | B-cells; SARS-CoV2 | 414 | EVQLVESGGGLVKPGGSLRLSCAASGFT | 1493 |
| 2394 | | | CoV1 | | SARS-CoV2 | | Human Patient | | FSGYSMNWVRQAPGKGLEWVSSISSSS | |
| | | | | | | | | | SYIYYADSVKGRFTISRDNAKNSLYLQMN | |
| | | | | | | | | | SLRAEDTAVYYCARWLQLRSDYYYFGM | |
| | | | | | | | | | DVWGQGTTVTVSS | |
| CoV2- | Ab | SARS-CoV1, | | | SARS-CoV2 | S; non- | B-cells; SARS-CoV2 | 415 | EVQLVESGGGLVQPGGSLRLSCVASGFT | 1494 |
| 2397 | | SARS-CoV2 | | | | RBD | Human Patient | | FSFYWMSWVRQAPGKGLEWVANIKQ | |
| | | | | | | | | | DGGEKYYVDSVKGRFTISRDNAKNSLYL | |
| | | | | | | | | | QMNSLRAEDTAVYYCARLSGSSWDFDY | |
| | | | | | | | | | WGQGTLVTVSS | |
| CoV2- | Ab | SARS-CoV2 | SARS- | | SARS-CoV1, | S; RBD | B-cells; SARS-CoV2 | 416 | EVQLVESGGGLVQPGGSLRLSCAASGFS | 1495 |
| 2399 | | | CoV1 | | SARS-CoV2 | | Human Patient | | VSTNYMSWVRQAPGKGLEWVSVIYSG | |
| | | | | | | | | | GSTFYADSVKGRFTISRDNSKNTLYLQM | |
| | | | | | | | | | NSLRAEDTAVYYCARDYRDWIWGQGTL | |
| | | | | | | | | | VTVSS | |
| CoV2- | Ab | SARS-CoV2 | SARS- | | SARS-CoV1, | S; non- | B-cells; SARS-CoV2 | 417 | QITLKESGPTLVKPTQTLTLTCTFSGFSLS | 1496 |
| 2400 | | | CoV1 | | SARS-CoV2 | RBD | Human Patient | | TSGVGVGWIRQPPGKALEWLALIYWD | |
| | | | | | | | | | DDKRYSPSLKSRLTITKDTSKNQVVLTMT | |
| | | | | | | | | | NMDPVDTATYYCAHNRFQYCSSTTCYTL | |
| | | | | | | | | | LPFDYWGQGTLVTVSS | |
| CoV2- | Ab | SARS-CoV1, | | | SARS-CoV2 | S; non- | B-cells; SARS-CoV2 | 418 | QVQLVESGGGVVQPGRSLRLSCAASGF | 1497 |
| 2401 | | SARS-CoV2 | | | | RBD | Human Patient | | TFSSYALFWVRQAPGKGLEWVAVISYD | |
| | | | | | | | | | GNNKYYADSVRGRFTISRDNSKNTLYLQ | |
| | | | | | | | | | MNSLRPEDTAVYYCARPYTGSYKSYMD | |
| | | | | | | | | | VWGKGTTVTVSS | |
| CoV2- | Ab | SARS-CoV1, | | | SARS-CoV2 | S; non- | B-cells; SARS-CoV2 | 419 | QVQLVESGGGVVQPGRSLRLSCAASGF | 1498 |
| 2403 | | SARS-CoV2 | | | | RBD | Human Patient | | TFSSYAMHWVRQAPGKGLEWVAVISY | |
| | | | | | | | | | DGSNKYYADSVKGRFTISRDNSKNTLYL | |
| | | | | | | | | | QMSSLRAEDTAVFYCARGDGDVYNFLL | |
| | | | | | | | | | VRNWFDPWGQGTLVTVSS | |
| CoV2- | Ab | SARS-CoV2 | SARS- | | SARS-CoV1, | S; NTD | B-cells; SARS-CoV2 | 420 | QLQLQESGPGLVKPSETLSLTCTVSGGSI | 1499 |
| 2405 | | | CoV1 | | SARS-CoV2 | | Human Patient | | SSSSYYWGWIRQPPGKGLEWIGSFYYSG | |
| | | | | | | | | | STYYNPSLKSRVTISVDTSKNQFSLNLSSV | |
| | | | | | | | | | TAADTAVYSCASLWFGDLYSFDYWGQG | |
| | | | | | | | | | TLVTVSS | |
| CoV2- | Ab | SARS-CoV1, | | | SARS-CoV2 | S; non- | B-cells; SARS-CoV2 | 421 | EVQLVESGGGLVQPGGSLRLSCADSAFT | 1500 |
| 2406 | | SARS-CoV2 | | | | RBD | Human Patient | | FSSFWMSWVRQAPGKGLEWVANIKQ | |
| | | | | | | | | | DGSEKFYLDSVKGRFTISRDNAKNSLYLQ | |
| | | | | | | | | | MNSLRAEDTAVYYCARLGRSSWNFDY | |
| | | | | | | | | | WGQGTLVTVSS | |
| CoV2- | Ab | SARS-CoV1, | | | SARS-CoV2 | S; non- | B-cells; SARS-CoV2 | 422 | QVQLQESGPGLVKPSQTLSLTCTVSGDSI | 1501 |
| 2408 | | SARS-CoV2 | | | | RBD | Human Patient | | SSGSYYWSWIRQPAGKGLEWIGRIYTSG | |
| | | | | | | | | | STTYNPSLKSRVTISVNTSKNQFSLNLSSV | |
| | | | | | | | | | TAADTAVYYCARVGGISPYYYYYYMDV | |
| | | | | | | | | | WGKGTTVTVSS | |
| CoV2- | Ab | SARS-CoV2 | SARS- | SARS-CoV2 | | S; RBD | B-cells; SARS-CoV2 | 423 | QVQLVQSGAEVKKPGASVKVSCKASGY | 1502 |
| 2413 | | | CoV1 | (weak) | | | Human Patient | | TFTSYDINWVRQATGQGLEWMGWMN | |
| | | | | | | | | | PNSGNAGYGQKFQGRVTMTRNTSISTA | |
| | | | | | | | | | YMELSSLRSEDTAVYYCARMRSGWPTH | |
| | | | | | | | | | GRPDDYWGRGTLVTVSS | |
| CoV2- | Ab | SARS-CoV2 | SARS- | | SARS-CoV1, | S; RBD | B-cells; SARS-CoV2 | 424 | QVQLQESGPGLVKPSQTLSLTCTVSGGSI | 1503 |
| 2416 | | | CoV1 | | SARS-CoV2 | | Human Patient | | SSGGYYWSWIRQHPGKGLEWIGYIYYS | |
| | | | | | | | | | GSTYYNPSLKSRVTISVDTSKNQFSLKLSS | |
| | | | | | | | | | VTAADTAVYYCASAKLVATISYFDYWGQ | |
| | | | | | | | | | GTLVTVSS | |
| CoV2- | Ab | SARS-CoV1, | | | SARS-CoV2 | S; RBD | B-cells; SARS-CoV2 | 425 | QVQLQQWGAGLLKPSETLSLTCAVYGG | 1504 |
| 2417 | | SARS-CoV2 | | | | | Human Patient | | SFSGYYWNWLRQPPGKGLEWIGEINHS | |
| | | | | | | | | | GSTNYNPSLKSRVTISVDTSKNQFSLKLSS | |
| | | | | | | | | | VTAADTAVYYCARVGGYYYYYMDVWG | |
| | | | | | | | | | KGTTVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | DIQMTQSPSSLSASVGDRVTITCRASQ | IGHV3-9 | IGHJ6 | IGKV1-39 | IGKJ1 | 2500 | AKDIIRQGED | 3702 | QQSYSTP | Seth Zost et al., 2020 |
| 2389 | NIASYLNWYQQKPGKAPKLLIYAASSL | (Human) | (Human) | (Human) | (Human) | | GMDV | | WT | (https://www.nature.co |
| | QSGVPSRFSGSGSGTDFTLTISSLQPEE | | | | | | | | | m/articles/s41591-020- |
| | FATYYCQQSYSTPWTFGQGTKVEIK | | | | | | | | | 0998-x) |
| CoV2- | QSVLTQAPSASGTPGQRVTISCSGSNS | IGHV1-8 | IGHJ4 | IGLV1-44 | IGLJ2 | 2501 | ARMRSGWPT | 3703 | AVWDDSL | Seth Zost et al., 2020 |
| 2391 | NIGSYTINWYQQLPGTAPKLLIYGNDQ | (Human) | (Human) | (Human) | (Human) | | HGRPDDF | | NGLV | (https://www.nature.co |
| | RTSGVPDRFSGSKFGTSASLAISGLQSE | | | | | | | | | m/articles/s41591-020- |
| | DENNYYCAVWDDSLNGLVFGGGTKL | | | | | | | | | 0998-x |
| | TVL | | | | | | | | | |
| CoV2- | EIVMTQSPATLSVSPGERATLSCRASQ | IGHV3-21 | IGHJ6 | IGKV3-15 | IGKJ1 | 2502 | ARWLQLRSD | 3704 | QQCYNWP | Seth Zost et al., 2020 |
| 2394 | SVSNNLAWYQHKPGQAPRLLIYGAST | (Human) | (Human) | (Human) | (Human) | | YYYFGMDV | | PWT | (https://www.nature.co |
| | RATGIPARFSGSGSGTEFTLTISSLQSE | | | | | | | | | m/articles/s41591-020- |
| | DFAVYFCQQCYNWPPWTFGQGTKVE | | | | | | | | | 0998-x) |
| | IK | | | | | | | | | |
| CoV2- | SYELTQPPSVSVSPGQTASITCSGDKLG | IGHV3-7 | IGHJ4 | IGLV3-1 | IGLJ1 | 2503 | ARLSGSSWDF | 3705 | QAWDSST | Seth Zost et al., 2020 |
| 2397 | DKYACWYQQRPGQSPVLVIYQDSKRP | (Human) | (Human) | (Human) | (Human) | | DY | | GV | (https://www.nature.co |
| | SGIPERFSGSNSGNTATLTISGTQAMD | | | | | | | | | m/articles/s41591-020- |
| | EADYYCQAWDSSTGVFGTGTKVTVL | | | | | | | | | 0998-x) |
| CoV2- | DIQMTQSPSSLSASVGDRVTITCQASQ | IGHV3-66 | IGHJ4 | IGKV1-33 | IGKJ1 | 2504 | ARDYRDWI | 3706 | QQYHNLP | Seth Zost et al., 2020 |
| 2399 | DISKYLNWYQQKPGKAPKLLIYDASNL | (Human) | (Human) | (Human) | (Human) | | | | RT | (https://www.nature.co |
| | ERGVPSRFSGSGSGTDFTFTISSLQAED | | | | | | | | | m/articles/s41591-020- |
| | IATYYCQQYHNLPRTFGQGTKVEIK | | | | | | | | | 0998-x) |
| CoV2- | DIQLTQSPSFLSASVRDRVTITCRASQG | IGHV2-5 | IGHJ4 | IGKV1-9 | IGKJ3 | 2505 | AHNRFQYCSS | 3707 | QQLNSYPF | Seth Zost et al., 2020 |
| 2400 | ISSYLAWYQQKPGKAPKLLIYAASTLQS | (Human) | (Human) | (Human) | (Human) | | TTCYTLLPFDY | | T | (https://www.nature.co |
| | GVPSRFSGSGSGTEFTLTISSLQPEDFA | | | | | | | | | m/articles/s41591-020- |
| | TYYCQQLNSYPFTFGPGTKVDIK | | | | | | | | | 0998-x) |
| CoV2- | DIVMTQSPDSLAVSLGERATINCKSSQ | IGHV3-30 | IGHJ6 | IGKV4-1 | IGKJ1 | 2506 | ARPYTGSYKS | 3708 | QQYYSISW | Seth Zost et al., 2020 |
| 2401 | SVLYSSNNKNSLAWYQQKPGQPPKLLI | (Human) | (Human) | (Human) | (Human) | | YMDV | | T | (https://www.nature.co |
| | YWASTRESGVPDRFSGSGSGTDFTLTI | | | | | | | | | m/articles/s41591-020- |
| | SSLQAEDVAVYYCQQYYSISWTFGQG | | | | | | | | | 0998-x) |
| | TKVEIK | | | | | | | | | |
| CoV2- | QSALTQPASVSGSPGQSITISCTGTSSD | IGHV3-30 | IGHJ5 | IGLV2-14 | IGLJ1 | 2507 | ARGDGDVYN | 3709 | SSYTSSST | Seth Zost et al., 2020 |
| 2403 | VGGYNYVSWYQQHPGKAPKLMIYDV | (Human) | (Human) | (Human) | (Human) | | FLLVRNWFDP | | LYV | (https://www.nature.co |
| | GNRPSGVSNRFSGSKSGNTASLTISGL | | | | | | | | | m/articles/s41591-020- |
| | QAEDEADYYCSSYTSSSTLYVFGTGTKV | | | | | | | | | 0998-x) |
| | TVL | | | | | | | | | |
| CoV2- | DIQMTQSPSSLSASVGDRVTFTCRASQ | IGHV4-39 | IGHJ4 | IGKV1-27 | IGKJ1 | 2508 | ASLWFGDLYS | 3710 | QKYNSAP | Seth Zost et al., 2020 |
| 2405 | GISNYLAWYQQKPGKVPKLLIYAASTL | (Human) | (Human) | (Human) | (Human) | | FDY | | WT | (https://www.nature.co |
| | QSGVPSRFSGIGSGTDFTLTISSLQPED | | | | | | | | | m/articles/s41591-020- |
| | VATYYCQKYNSAPWTFGQGTKVEIK | | | | | | | | | 0998-x) |
| CoV2- | SYELTQPPSVSVSPGQTASITCSGDKLG | IGHV3-7 | IGHJ4 | IGLV3-1 | IGLJ2 | 2509 | ARLGRSSWN | 3711 | QAWDSST | Seth Zost et al., 2020 |
| 2406 | DKFVCWYQQKPGQSPVLVIYQDNKR | (Human) | (Human) | (Human) | (Human) | | FDY | | GV | (https://www.nature.co |
| | PSGIPERFSGSNSGNTATLTISGTQALD | | | | | | | | | m/articles/s41591-020- |
| | EADYYCQAWDSSTGVFGGGTKLTVL | | | | | | | | | 0998-x) |
| CoV2- | DIQLTQSPSFLSASVGDRVTITCRASQG | IGHV4-61 | IGHJ6 | IGKV1-9 | IGKJ5 | 2510 | ARVGGISPYY | 3712 | QQLNSYPI | Seth Zost et al., 2020 |
| 2408 | ISSYLAWYQQKPGKAPKLLMYAASTL | (Human) | (Human) | (Human) | (Human) | | YYYYMDV | | T | (https://www.nature.co |
| | QSGVPSRFSGSGSGTEFTLTINTLQPE | | | | | | | | | m/articles/s41591-020- |
| | DFATYYCQQLNSYPITFGQGTRLEIK | | | | | | | | | 0998-x) |
| CoV2- | QSVLTQPPSASGTPGQRVTFSCSGSNS | IGHV1-8 | IGHJ4 | IGLV1-44 | IGLJ3 | 2511 | ARMRSGWPT | 3713 | AVWDDSL | Seth Zost et al., 2020 |
| 2413 | NIGSYTVNWYQQLPGTAPKLLIYDNN | (Human) | (Human) | (Human) | (Human) | | HGRPDDY | | NGLV | (https://www.nature.co |
| | QRPSGVPDRFSGSKSGTSASLAISGLQ | | | | | | | | | m/articles/s41591-020- |
| | SEDEADYYCAVWDDSLNGLVFGGGT | | | | | | | | | 0998-x) |
| | KLTVL | | | | | | | | | |
| CoV2- | QSVLTQPPSVSEAPRQRVTISCSGSSS | IGHV4-31 | IGHJ4 | IGLV1-36 | IGLJ2 | 2512 | ASAKLVATISY | 3714 | AAWDDSL | Seth Zost et al., 2020 |
| 2416 | NIGNNAVNWYQQLPGKAPKLLIYYDD | (Human) | (Human) | (Human) | (Human) | | FDY | | NGVV | (https://www.nature.co |
| | LLPSGVSDRFSGSKSGTSASLAISGLQS | | | | | | | | | m/articles/s41591-020- |
| | EDEADYYCAAWDDSLNGVVFGGGTK | | | | | | | | | 0998-x) |
| | LTVL | | | | | | | | | |
| CoV2- | DIQMTQSPSSLSASVGDRVTITCRASQ | IGHV4-34 | IGHJ6 | IGKV1-39 | IGKJ4 | 2513 | ARVGGYYYYY | 3715 | QQSYTTLL | Seth Zost et al., 2020 |
| 2417 | SITSYLNWYQQRPGKAPKLLIYAASSLQ | (Human) | (Human) | (Human) | (Human) | | MDV | | T | (https://www.nature.co |
| | SGVPSRFSGSGSGTDFTLSISSLQPEDF | | | | | | | | | m/articles/s41591-020- |
| | ATYYCQQSYTTLLTFGGGTKVEIK | | | | | | | | | 0998-x) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV2 | SARS- | | SARS-CoV1, | S; non- | B-cells; SARS-CoV2 | 426 | EVRLLESGGGLVQPGGSLRLSCAASGFT | 1505 |
| 2418 | | | CoV1 | | SARS-CoV2 | RBD | Human Patient | | FSDYAMNWVRQAPGKGLEWVSAISAT | |
| | | | | | | | | | GGSTFYADSVKGRFSISRDNSKNSLVLQ | |
| | | | | | | | | | MNSLRAEDTAVYYCAKPYGMDVWGQ | |
| | | | | | | | | | GTTVTVSS | |
| CoV2- | Ab | SARS-CoV2 | SARS- | | SARS-CoV1, | S; non- | B-cells; SARS-CoV2 | 427 | QGQLVQSGGDVVQPGKSLRLSCAASGF | 1506 |
| 2420 | | | CoV1 | | SARS-CoV2 | RBD | Human Patient | | TFTNYAMHWVRQAPGKGLEWVAVISN | |
| | | | | | | | | | DGSNEKYVDSVKGRFSLSRDNSKNTVYL | |
| | | | | | | | | | DMHSLRPEDTAIYYCARDRSNLERLVMT | |
| | | | | | | | | | FGGIIAGAFDIWGQGARVTVSS | |
| CoV2- | Ab | SARS-CoV1, | | | SARS-CoV2 | S; non- | B-cells; SARS-CoV2 | 428 | QVQLVESGGGVVQPGRSLRLSCAASGF | 1507 |
| 2422 | | SARS-CoV2 | | | | RBD | Human Patient | | TFSSYAMYWVRQAPGKGLEWVAVISYD | |
| | | | | | | | | | GINKYYADSVKGRFTISRDNSKNTLYLQ | |
| | | | | | | | | | MNSLRAEDTAVYYCARVNSGSYYSYFDY | |
| | | | | | | | | | WGQGTLVTVSS | |
| CoV2- | Ab | SARS-CoV2 | SARS- | | SARS-CoV1, | S; non- | B-cells; SARS-CoV2 | 429 | QVQLQESGPGLVKPSGTLSLTCAVSGGS | 1508 |
| 2427 | | | CoV1 | | SARS-CoV2 | RBD | Human Patient | | ISSSNWWSWVRQPPGKGLEWIGEIYHS | |
| | | | | | | | | | GSTNYNPSLKSRVTISVDKSKNQFSLKLN | |
| | | | | | | | | | SVTAADTAVYYCASRWGDYFDSSGAYD | |
| | | | | | | | | | SWGQGTLLTVSS | |
| CoV2- | Ab | SARS-CoV2 | SARS- | | SARS-CoV1, | S; non- | B-cells; SARS-CoV2 | 430 | QVQLVESGGGVVQPGKSLRLSCAASGF | 1509 |
| 2428 | | | CoV1 | | SARS-CoV2 | RBD | Human Patient | | TFSSYGMHWVRQAPGKGLEWVAVIWY | |
| | | | | | | | | | DGNNKFYVDSVKGRFTISRDNSKNTLY | |
| | | | | | | | | | MEMNSLRAEDTAVYYCARKGPLWRFD | |
| | | | | | | | | | YWGQGTLVTVSS | |
| CoV2- | Ab | SARS-CoV1, | | | SARS-CoV2 | S; non- | B-cells; SARS-CoV2 | 431 | EVQLVESGGGLIQPGGSLRLSCAASGFIV | 1510 |
| 2429 | | SARS-CoV2 | | | | RBD | Human Patient | | SSNYMSWVRQAPGKGLEWVSVIYSGG | |
| | | | | | | | | | STYYADSVKGRFTISRDNSKNTLYLQMN | |
| | | | | | | | | | SLRAEDTAVYYCARESTQWGQGTLVTV | |
| | | | | | | | | | SS | |
| CoV2- | Ab | SARS-CoV1, | | | SARS-CoV2 | S; non- | B-cells; SARS-CoV2 | 432 | QLQLQESGPGLVKPSETLSLTCTVSGGSI | 1511 |
| 2430 | | SARS-CoV2 | | | | RBD | Human Patient | | SSSSYYWGWIRQPPGKGLEWIGSVYYIG | |
| | | | | | | | | | STYYNPSLKSRVTMSVDTSKNQFSLKLSS | |
| | | | | | | | | | VTAADTAVYYCARAPFQLLDKYYFFYYM | |
| | | | | | | | | | DVWGKGTTVTVSS | |
| CoV2- | Ab | SARS-CoV2 | SARS- | | SARS-CoV1, | S; RBD | B-cells; SARS-CoV2 | 433 | EVQLVESGGDLIQPGGSLRLSCAASGLT | 1512 |
| 2434 | | | CoV1 | | SARS-CoV2 | | Human Patient | | VSSNYMSWVRQAPGKGLEWVSIIYSGG | |
| | | | | | | | | | STYYADSVKGRFSISRDNSNNTLYLQMN | |
| | | | | | | | | | SLRAEDTAVYFCARHIPAWGYKWGQGT | |
| | | | | | | | | | LVTVSS | |
| CoV2- | Ab | SARS-CoV2 | SARS- | | SARS-CoV1, | S; NTD | B-cells; SARS-CoV2 | 434 | EVQLLESGGGLVQPGGSLRLSCAASGFT | 1513 |
| 2438 | | | CoV1 | | SARS-CoV2 | | Human Patient | | FTSYGMSWVRQAPGKGLEWVSAISISG | |
| | | | | | | | | | GSTYYADSVKGRFTISRDNSKNTLYLQM | |
| | | | | | | | | | NSLRAEDTAVYYCAKLLGSGITLDNDAF | |
| | | | | | | | | | DIWGQGTMVTVSS | |
| CoV2- | Ab | SARS-CoV1, | | | SARS-CoV2 | S; non- | B-cells; SARS-CoV2 | 435 | QVQLVQSGAEVKKPGSSVKVSCKASGG | 1514 |
| 2441 | | SARS-CoV2 | | | | RBD | Human Patient | | TFSSYAIIWVRQAPGQGLEWMGGIIPIF | |
| | | | | | | | | | GTTNYAQKFQGRVTITADESTSTAYVELS | |
| | | | | | | | | | SLRSEDTAVYYCARIGHFDSSGYYLDYW | |
| | | | | | | | | | GQGTLVTVSS | |
| CoV2- | Ab | SARS-CoV2 | SARS- | | SARS-CoV1, | S; NTD | B-cells; SARS-CoV2 | 436 | EVQLLESGGGLVQPGGSLRLSCAASGFT | 1515 |
| 2444 | | | CoV1 | | SARS-CoV2 | | Human Patient | | FTSYAMNWVRQAPGKGLEWVSAISVS | |
| | | | | | | | | | GGSTYYADSVKGRFTISRDNSKNTLYLQ | |
| | | | | | | | | | MNSLRAEDTAVYYCAKDFGSGIVGATG | |
| | | | | | | | | | FDFWGQGTLVTVSS | |
| CoV2- | Ab | SARS-CoV2 | SARS- | | SARS-CoV1, | S; non- | B-cells; SARS-CoV2 | 437 | EVQLVESGGGVVRPGGSLRLSCAASGFT | 1516 |
| 2445 | | | CoV1 | | SARS-CoV2 | RBC | Human Patient | | FDDYGMSWVRQAPGKGLEWVSAINW | |
| | | | | | | | | | NGGSTGYADSVKGRFTISRDNAKNSLYL | |
| | | | | | | | | | QMNSLRAEDTALYHCARRRSSSRYSSG | |
| | | | | | | | | | WYMYYYYMDVWGKGTTVTVSS | |
| CoV2- | Ab | SARS-CoV2 | SARS- | | SARS-CoV1, | S; non- | B-cells; SARS-CoV2 | 438 | EVQLVQSGAEVKKPGESLKISCKGSGYSF | 1517 |
| 2446 | | | CoV1 | | SARS-CoV2 | RBD | Human Patient | | TNSWIGWVRQMPGKGLEWMGIIYPG | |
| | | | | | | | | | DSDTRYSPSFQGQVTISADKSISTAYLQW | |
| | | | | | | | | | SSLKASDTAIYYCATHRCSGGFCYLAYWG | |
| | | | | | | | | | QGTLVTVSS | |
| CoV2- | Ab | SARS-CoV1, | | | SARS-CoV2 | S; non- | B-cells; SARS-CoV2 | 439 | QVQLVESGGGVVQPGRSLRLSCATSGFT | 1518 |
| 2449 | | SARS-CoV2 | | | | RBD | Human Patient | | FSSFALHWVRQAPGKGLEWVTVISDDG | |
| | | | | | | | | | NNKYYVDSVKGRFTISRDNSKNTLFLQM | |
| | | | | | | | | | NSLRVEDTAIYYCARASYNSNWSIGEYFR | |
| | | | | | | | | | DWGQGTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | QSALTQPASVSGSPGQSITISCTGTSSD | IGHV3-23 | IGHJ6 | IGLV2-14 | IGLJ3 | 2514 | AKPYGMDV | 3716 | SSYTSTSTP | Seth Zost et al., 2020 |
| 2418 | VGGYNFVSWHQQHPGKAPKNMIYD | (Human) | (Human) | (Human) | (Human) | | | | WV | (https://www.nature.co |
| | VSKRPSGVSNRFSGSKSGNTASLTISGL | | | | | | | | | m/articles/s41591-020- |
| | HTEDEADYYCSSYTSTSTPWVFGGGTK | | | | | | | | | 0998-x) |
| | LTVL | | | | | | | | | |
| CoV2- | EIVMTQSPGTLSVSPGERATLSCRASQ | IGHV3-30 | IGHJ3 | IGKV3-15 | IGKJ5 | 2515 | ARDRSNLERL | 3717 | QQYHVWP | Seth Zost et al., 2020 |
| 2420 | SLSSHLAWYQQKPGQAPRLLIYGVSTR | (Human) | (Human) | (Human) | (Human) | | VMTFGGIIAG | | PIT | (https://www.nature.co |
| | ATGIPARFSGSGSGTEFTLAISSLQSED | | | | | | AFDI | | | m/articles/s41591-020- |
| | SAVYYCQQYHVWPPITFGQGTRLEIK | | | | | | | | | 0998-x) |
| CoV2- | DIVMTQSPDSLAVSLGERATINCKSSQ | IGHV3-30 | IGHJ4 | IGKV4-1 | IGKJ1 | 2516 | ARVNSGSYYS | 3718 | QQYYSTPL | Seth Zost et al., 2020 |
| 2422 | SVLYSSNNKNYLAWYQQKPGQPPKLLI | (Human) | (Human) | (Human) | (Human) | | YFDY | | T | (https://www.nature.co |
| | YWASTRESGVPDRFSGSGSGTDFTLTI | | | | | | | | | m/articles/s41591-020- |
| | SSLQAEDVAVYYCQQYYSTPLTFGQGT | | | | | | | | | 0998-x) |
| | KVEIK | | | | | | | | | |
| CoV2- | DIQMTQSPSSVSASVGDRVTITCRASQ | IGHV4-4 | IGHJ4 | IGKV1-12 | IGKJ4 | 2517 | ASRWGDYFD | 3719 | QQGNSFPL | Seth Zost et al., 2020 |
| 2427 | GISSWLAWYQQKPGKAPKLLIYAASSL | (Human) | (Human) | (Human) | (Human) | | SSGAYDS | | T | (https://www.nature.co |
| | QSGVPSRFSGSGSGTDFTLTITSLQPED | | | | | | | | | m/articles/s41591-020- |
| | FATYYCQQGNSFPLTFGGGTKVEIK | | | | | | | | | 0998-x) |
| CoV2- | QSVLTQPPSVSGAPGQRVTISCTGSSS | IGHV3-33 | IGHJ4 | IGLV1-40 | IGLJ3 | 2518 | ARKGPLWRF | 3720 | QSYDSSLS | Seth Zost et al., 2020 |
| 2428 | NIGAGYDVHWYQQIPGTAPKLLFYGN | (Human) | (Human) | (Human) | (Human) | | DY | | AWV | (https://www.nature.co |
| | NNRPSGVPDRFSDSKSGTSASLAITGL | | | | | | | | | m/articles/s41591-020- |
| | QAEDEADYYCQSYDSSLSAWVFGGGT | | | | | | | | | 0998-x) |
| | KLTVL | | | | | | | | | |
| CoV2- | DIQMTQSPSTLSASVGDRVTITCRASH | IGHV3-53 | IGHJ4 | IGKV1-5 | IGKJ1 | 2519 | ARESTQ | 3721 | QQYNTYS | Seth Zost et al., 2020 |
| 2429 | SISSWLAWYQQKPGKAPKLLIYKASSL | (Human) | (Human) | (Human) | (Human) | | | | QT | (https://www.nature.co |
| | ESGVPSRFSGSGSGTEFTLTISSLQPDD | | | | | | | | | m/articles/s41591-020- |
| | FATYYCQQYNTYSQTFGQGTKVEIK | | | | | | | | | 0998-x) |
| CoV2- | EIVLTQSPATLSLSPGERATLSCRASQS | IGHV4-39 | IGHJ6 | IGKV3-11 | IGKJ5 | 2520 | ARAPFQLLDK | 3722 | QQRSNWP | Seth Zost et al., 2020 |
| 2430 | VSSYLAWYQQKPGQAPRLLIYDASNR | (Human) | (Human) | (Human) | (Human) | | YYFFYYMDV | | PGVT | (https://www.nature.co |
| | ATGIPARFSGSGSGTDFTLTISSLEPEDF | | | | | | | | | m/articles/s41591-020- |
| | AVYYCQQRSNWPPGVTFGQGTRLEIK | | | | | | | | | 0998-x) |
| CoV2- | DIQMTQSPSSLSASVGDRVTITCQASQ | IGHV3-53 | IGHJ4 | IGKV1-33 | IGKJ2 | 2521 | ARHIPAWGY | 3723 | HQYDYLPY | Seth Zost et al., 2020 |
| 2434 | DIRNYLNWYQQKPGKAPELLIYDASNL | (Human) | (Human) | (Human) | (Human) | | K | | T | (https://www.nature.co |
| | ETGVPSRFSGSGSGTDFIFTISSLQPEDI | | | | | | | | | m/articles/s41591-020- |
| | ATYYCHQYDYLPYTFGQGTKLDIK | | | | | | | | | 0998-x) |
| CoV2- | DIQMTQSPSSLSASVGDRVTITCRASQ | IGHV3-23 | IGHJ3 | IGKV1-39 | IGKJ2 | 2522 | AKLLGSGITL | 3724 | QQTYSTPL | Seth Zost et al., 2020 |
| 2438 | SISSYLNWYQQKPGKAPKLLIYAATSLQ | (Human) | (Human) | (Human) | (Human) | | DNDAFDI | | YT | (https://www.nature.co |
| | SGVPSRFSGSGSGTDFTLTISSLQPEDF | | | | | | | | | m/articles/s41591-020- |
| | ATYYCQQTYSTPLYTFGQGTKLEIK | | | | | | | | | 0998-x) |
| CoV2- | EIVLTQSPATLSLSPGERATLSCRASQS | IGHV1-69 | IGHJ4 | IGKV3-11 | IGKJ3 | 2523 | ARIGHFDSSG | 3725 | QHRTNWP | Seth Zost et al., 2020 |
| 2441 | VSSFLAWYQQKPGQAPRLLIYDASNR | (Human) | (Human) | (Human) | (Human) | | YYLDY | | PLFT | (https://www.nature.co |
| | PTGIPARFTGSGSGTDFTLTISSLEPEDF | | | | | | | | | m/articles/s41591-020- |
| | AVYYCQHRTNWPPLFTFGPGTKVDIK | | | | | | | | | 0998-x) |
| CoV2- | SYELTQPPSVSVSPGQTARITCSGDALP | IGHV3-23 | IGHJ4 | IGLV3-25 | IGLJ2 | 2524 | AKDFGSGIVG | 3726 | QSADSSGT | Seth Zost et al., 2020 |
| 2444 | NQYAYWYQQKPGQAPVLVMCKDSE | (Human) | (Human) | (Human) | (Human) | | ATGFDF | | YVV | (https://www.nature.co |
| | RPSGIPERFSGSSSGTTVTLTISGVQAE | | | | | | | | | m/articles/s41591-020- |
| | DEADYYCQSADSSGTYVVFGGGTKLT | | | | | | | | | 0998-x) |
| | VL | | | | | | | | | |
| CoV2- | DIQMTQSPSTLSASVGDRVTITCRASQ | IGHV3-20 | IGHJ6 | IGKV1-5 | IGKJ1 | 2525 | ARRRSSSRYS | 3727 | QQYNTYSG | Seth Zost et al., 2020 |
| 2445 | SVSTWLAWYQQKPGKAPNLLIYEASSL | (Human) | (Human) | (Human) | (Human) | | SGWYMYYYY | | T | (https://www.nature.co |
| | ESGVPSRFSGSGSGTEFTLTISSLQPDD | | | | | | MDV | | | m/articles/s41591-020- |
| | FATYYCQQYNTYSGTFGQGTKVEIK | | | | | | | | | 0998-x) |
| CoV2- | QPVLTQPPSASASLGASVTLTCTLSSGY | IGHV5-51 | IGHJ4 | IGLV9-49 | IGLJ2 | 2526 | ATHRCSGGFC | 3728 | GADHGSG | Seth Zost et al., 2020 |
| 2446 | SNYKVDWYQQRPGKGPRFVMRVGT | (Human) | (Human) | (Human) | (Human) | | YLAY | | SNFVFVV | (https://www.nature.co |
| | GGIVGSKGDGIPDRFSVLGSGLNRYLTI | | | | | | | | | m/articles/s41591-020- |
| | KNIQEEDESDYHCGADHGSGSNFVFV | | | | | | | | | 0998-x) |
| | VFGGGTKLTVL | | | | | | | | | |
| CoV2- | DIVMTQSPDSLAVSLGERATINCKSSQ | IGHV3-30 | IGHJ1 | IGKV4-1 | IGKJ1 | 2527 | ARASYNSNW | 3729 | QQYYSPP | Seth Zost et al., 2020 |
| 2449 | SLLYTSNNKNYLAWYQQKPGQPPKLLI | (Human) | (Human) | (Human) | (Human) | | SIGEYFRD | | WT | (https://www.nature.co |
| | YWASTRESGVPDRFSGSGSGTDFTLTI | | | | | | | | | m/articles/s41591-020- |
| | SSLQAEDVAVYYCQQYYSPPWTFGQG | | | | | | | | | 0998-x) |
| | TKVEIK | | | | | | | | | |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV1, | | | SARS-CoV2 | S; RBD | B-cells; SARS-CoV2 | 440 | QVQLVESGGGVVQPGRSLRLSCAASGF | 1519 |
| 2450 | | SARS-CoV2 | | | | | Human Patient | | TFSTYGMHWVRQAPGKGLEWVAVILY | |
| | | | | | | | | | DGSNRYYADSVKGRFTISRDNSKNTLYL | |
| | | | | | | | | | QMNSLRAEDTAVYYCAKQGGLYCSGTN | |
| | | | | | | | | | CWGGYLDYWGQGTLVTVSS | |
| CoV2- | Ab | SARS-CoV2 | SARS- | | SARS-CoV1, | S; non- | B-cells; SARS-CoV2 | 441 | QITLKESGPTLVKPTQTLTLTCTVSGFSLS | 1520 |
| 2451 | | | CoV1 | | SARS-CoV2 | RBD | Human Patient | | TSGVGVGCIRQPPGKALEWLALIYWDD | |
| | | | | | | | | | DKRYSPSLKSRLTITRDTSKNQVVLTMTN | |
| | | | | | | | | | MDPVDTGTYFCVHRHVSGAFDYWGQG | |
| | | | | | | | | | TLVTVSS | |
| CoV2- | Ab | SARS-CoV1, | | | SARS-CoV2 | S; non- | B-cells; SARS-CoV2 | 442 | QVQLVQSGSELKKPGASVKVSCKASGYT | 1521 |
| 2453 | | SARS-CoV2 | | | | RBD | Human Patient | | FTSYAMNWVRQAPGQGLEWMGWINT | |
| | | | | | | | | | NTGNPTYAQGFTGRFVFSLDTSVNTAYL | |
| | | | | | | | | | QISSLKAEDTAVYYCARARLLGYCSSTSCY | |
| | | | | | | | | | TIGWGAFDIWGQGTMVTVSS | |
| CoV2- | Ab | SARS-CoV2 | SARS- | | SARS-CoV1, | S; non- | B-cells; SARS-CoV2 | 443 | QVQLVESGGGVVQPGRSLRLSCAASGF | 1522 |
| 2454 | | | CoV1 | | SARS-CoV2 | RBD | Human Patient | | TFSSYGMHWVRQAPGKGLEWVAVIWY | |
| | | | | | | | | | DGSNKYYADSVKGRFTISRDNSKNTLYL | |
| | | | | | | | | | QMNSLRAEDTAVYYCAREGQGTYLDY | |
| | | | | | | | | | WGQGTLVTVSS | |
| CoV2- | Ab | SARS-CoV2 | SARS- | | SARS-CoV1, | S; NTD | B-cells; SARS-CoV2 | 444 | EVQLVESGGGLVQPGGSLRLSCAASGFT | 1523 |
| 2455 | | | CoV1 | | SARS-CoV2 | | Human Patient | | FSSYEMINWVRQAPGKGLEWVSYISSSG | |
| | | | | | | | | | SAIYYADSVKGRFTISRDNAKNSLYLQM | |
| | | | | | | | | | INSLRVEDTAVYYCAREARSRYFDWLPSY | |
| | | | | | | | | | YFDYWGQGTLVTVSS | |
| CoV2- | Ab | SARS-CoV1, | | | SARS-CoV2 | S; non- | B-cells; SARS-CoV2 | 445 | QVQLVESGGGVVQPGRSLRLSCAASGF | 1524 |
| 2458 | | SARS-CoV2 | | | | RBD | Human Patient | | TFSRHAMHWVRQAPGKGLEWVAVISY | |
| | | | | | | | | | DGSNKYYADSVKGRFAISRDNSKNTLYL | |
| | | | | | | | | | QMNSLRPEDTAVYYCARDPSPLVLITSID | |
| | | | | | | | | | YWGQGTLVTVSS | |
| CoV2- | Ab | SARS-CoV2 | SARS- | | SARS-CoV1, | S; NTD | B-cells; SARS-CoV2 | 446 | QLQLQESGPGLVKPSETLSLTCTVSGGSI | 1525 |
| 2459 | | | CoV1 | | SARS-CoV2 | | Human Patient | | SSGTYYCGWIRQPPGKGLEWIGSTYYG | |
| | | | | | | | | | GSTLYNPSLRGRVTISVDTSKNQFSLKLSS | |
| | | | | | | | | | VTAADTAVYYCARRGNYYDSKNWFDP | |
| | | | | | | | | | WGQGTLVTVSS | |
| CoV2- | Ab | SARS-CoV1, | | | SARS-CoV2 | S; RBD | B-cells; SARS-CoV2 | 447 | QVQLQESGPGLVKPSQTLSLTCTVSGGSI | 1526 |
| 2461 | | SARS-CoV2 | | | | | Human Patient | | SSGGYFWSWIRQHPGKGLEWIGSIYYS | |
| | | | | | | | | | GSTYYNPSLRSRITISVDTSKNQFSLKLSS | |
| | | | | | | | | | VTAADTAVYYCARGGSGSYSLFDYWGQ | |
| | | | | | | | | | GTLVTVSS | |
| CoV2- | Ab | SARS-CoV1, | | | SARS-CoV2 | S; RBD | B-cells; SARS-CoV2 | 448 | EVQLVESGGGLVQPGGSLRLSCAASGFT | 1527 |
| 2462 | | SARS-CoV2 | | | | | Human Patient | | FSSSDLHWVRQATGKGLEWVSAIGTAG | |
| | | | | | | | | | DTYYLGSVKGRFTISRENGKNSLYLQMN | |
| | | | | | | | | | SLRAGDTAVYYCARVLYDSSGFYNWFDP | |
| | | | | | | | | | WGQGTLVTVSS | |
| CoV2- | Ab | SARS-CoV1, | | | SARS-CoV2 | S; RBD | B-cells; SARS-CoV2 | 449 | QVQLQESGPGLVKPSQTLSLTCTVSGGSI | 1528 |
| 2464 | | SARS-CoV2 | | | | | Human Patient | | SSGGYYWSWIRQHPGKGLEWIGYISYS | |
| | | | | | | | | | GSTYYNPSLKSRLTISVDTSKNQFSLKLSS | |
| | | | | | | | | | VTAADTAVYYCARDLGDGYNLRVPAYF | |
| | | | | | | | | | DLWGRGTLVTVSS | |
| CoV2- | Ab | SARS-CoV1, | | | SARS-CoV2 | S; RBD | B-cells; SARS-CoV2 | 450 | QVQLQESGPGLVKPSGTLSLTCAVSGGS | 1529 |
| 2465 | | SARS-CoV2 | | | | | Human Patient | | ISSSNWWSWVRQPPGKGLEWIGEIYHG | |
| | | | | | | | | | GSTDYNPSLKSRVTISVDKSKNQFSLKLTS | |
| | | | | | | | | | VTAADTAVYYCARVDHVNVRDYWGPG | |
| | | | | | | | | | TLVTVSS | |
| CoV2- | Ab | SARS-CoV2 | SARS- | | SARS-CoV1, | S; NTD | B-cells; SARS-CoV2 | 451 | EVQLVESGGGLVKPGGSLRLSCAASGFT | 1530 |
| 2466 | | | CoV1 | | SARS-CoV2 | | Human Patient | | FSSYSMNWVRQAPGKGLEWVSSISNSN | |
| | | | | | | | | | SFIYYADSMKGRFTISRDNAKNSLYLQM | |
| | | | | | | | | | NSLRAEDTAVYYCARVNGNSNWNFGSY | |
| | | | | | | | | | YYYYMDVWGKGTTVTVSS | |
| CoV2- | Ab | SARS-CoV2 | SARS- | | SARS-CoV1, | S; non- | B-cells; SARS-CoV2 | 452 | QVQLVESGGGVVQPGRSLRLSCAASGF | 1531 |
| 2473 | | | CoV1 | | SARS-CoV2 | RBD | Human Patient | | TFSNYGMHWVRQAPGKGLEWVAVIYY | |
| | | | | | | | | | DGSNKYYADSVKGRFTISRDNSKNTLYL | |
| | | | | | | | | | QIHSLRAEDTAVYYCAREGQMAATTGID | |
| | | | | | | | | | YWGQGTLVTVSS | |
| CoV2- | Ab | SARS-CoV2 | SARS- | | SARS-CoV1, | S; non- | B-cells; SARS-CoV2 | 453 | EVQLVESGGGLVKPGGSLRLSCAASGFT | 1532 |
| 2474 | | | CoV1 | | SARS-CoV2 | RBD | Human Patient | | FSNAWMSWVRQAPGKGLEWVGRIKG | |
| | | | | | | | | | KTDGGTTDYAAPVKGRFTISKDDSKNTL | |
| | | | | | | | | | YLQMSSLNTEDTAVYWCTTLTYYYDSSA | |
| | | | | | | | | | YLNDAFDIWGQGTMVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | DIQMTQSPSSLSASVGDRVTITCQASQ | IGHV3-30 | IGHJ4 | IGKV1-33 | IGKJ5 | 2528 | AKQGGLYCSG | 3730 | QQYHNLP | Seth Zost et al., 2020 |
| 2450 | DISNYLHWYQQKPGKAPKLLIYDASNL | (Human) | (Human) | (Human) | (Human) | | TNCWGGYLD | | PIT | (https://www.nature.co |
| | ETGVPSRFSGSGSGTDFTFTISSLQPED | | | | | | Y | | | m/articles/s41591-020- |
| | IATYYCQQYHNLPPITFGQGTRLEIK | | | | | | | | | 0998-x) |
| CoV2- | SYELTQPPSVSVSPGQTASITCSGDKLG | IGHV2-5 | IGHJ4 | IGLV3-1 | IGLJ1 | 2529 | VHRHVSGAF | 3731 | QAWDSST | Seth Zost et al., 2020 |
| 2451 | ETYVSWYQQKPGQSPVLVIYEDSKRPS | (Human) | (Human) | (Human) | (Human) | | DY | | GGI | (https://www.nature.co |
| | GIPERFSGSNSGNTATLTISGTQAMDE | | | | | | | | | m/articles/s41591-020- |
| | ADYYCQAWDSSTGGVFGTGTKVTVL | | | | | | | | | 0998-x) |
| CoV2- | DIQMTQSPSSLSASVGDRVTITCQASL | IGHV7-4- | IGHJ3 | IGKV1-33 | IGKJ5 | 2530 | ARARLLGYCS | 3732 | QQYDNLP | Seth Zost et al., 2020 |
| 2453 | DISKYLNWYQHKPGKAPNLLIYDAFNL | 1 | (Human) | (Human) | (Human) | | STSCYTIGWG | | PGVSTT | (https://www.nature.co |
| | ERGVPSRFSGSGSGTDFTFTISSLQPED | (Human) | | | | | AFDI | | | m/articles/s41591-020- |
| | IATYYCQQYDNLPPGVSTTFGQGTRLE | | | | | | | | | 0998-x) |
| | IK | | | | | | | | | |
| CoV2- | SYELTQPPSVSVSPGQTARITCSGDALP | (Human) | IGHJ4 | IGLV3-10 | IGLJ3 | 2531 | AREGQGTYLD | 3733 | CSTDSSGN | Seth Zost et al., 2020 |
| 2454 | KKYAHWYQQKSGQAPVLVIYEDSKRP | IGHV3-33 | (Human) | (Human) | (Human) | | Y | | QRV | (https://www.nature.co |
| | SGIPERFSGSSSGTMATLTISGAQVED | | | | | | | | | m/articles/s41591-020- |
| | EADYYCCSTDSSGNQRVFGGGTKLTVL | | | | | | | | | 0998-x) |
| CoV2- | QSALTQPASVSGSPGQSITISCTGTSSD | IGHV3-48 | IGHJ4 | IGLV2-14 | IGLJ2 | 2532 | AREARSRYFD | 3734 | SSYTSSST | Seth Zost et al., 2020 |
| 2455 | IGGYNYVSWYQQHPGKAPKLLIYDVS | (Human) | (Human) | (Human) | (Human) | | WLPSYYFDY | | HVV | (https://www.nature.co |
| | NRPSGVSTRFSGSKSGNTASLTISGLQ | | | | | | | | | m/articles/s41591-020- |
| | AEDEADYYCSSYTSSSTHVVFGGGTKL | | | | | | | | | 0998-x) |
| | TVL | | | | | | | | | |
| CoV2- | SYELTQPPSVSVSPGQTARITCSGDALP | IGHV3-30 | IGHJ4 | IGLV3-25 | IGLJ2 | 2533 | ARDPSPLVLIT | 3735 | QSADTIGT | Seth Zost et al., 2020 |
| 2458 | RQYTYWYQQKPGQAPVLVIYKDSERP | (Human) | (Human) | (Human) | (Human) | | SIDY | | YWV | (https://www.nature.co |
| | SGIPERFSGSSSGTTVTLTISGVQAEDE | | | | | | | | | m/articles/s41591-020- |
| | ADYYCQSADTIGTYWVFGGGTKLTVL | | | | | | | | | 0998-x) |
| CoV2- | EIVMTQSPATVSVSPGERATLSCRASQ | IGHV4-39 | IGHJ5 | IGKV3-15 | IGKJ2 | 2534 | ARRGNYYDSK | 3736 | QQYNNWP | Seth Zost et al., 2020 |
| 2459 | SVSSNLAWYQQKPGQAPRLLIYGAST | (Human) | (Human) | (Human) | (Human) | | NWFDP | | PMYT | (https://www.nature.co |
| | RATGIPARFSASGSGTEFTLTISSLQSED | | | | | | | | | m/articles/s41591-020- |
| | FAVYYCQQYNNWPPMYTFGQGTKVE | | | | | | | | | 0998-x) |
| | IK | | | | | | | | | |
| CoV2- | DIQMTQSPSSLSASVGDRVTITCQASQ | IGHV4-31 | IGHJ4 | IGKV1-33 | IGKJ2 | 2535 | ARGGSGSYSL | 3737 | QQYDNLYS | Seth Zost et al., 2020 |
| 2461 | DITNYLNWYQQKPGKAPKLLIYDASNL | (Human) | (Human) | (Human) | (Human) | | FDY | | VH | (https://www.nature.co |
| | ETGVPSRFSGSGSGTDFTFTISSLQPED | | | | | | | | | m/articles/s41591-020- |
| | FATYYCQQYDNLYSVHFGQGTKLEIK | | | | | | | | | 0998-x) |
| CoV2- | DIQMTQSPSSLSASVGDRVTITCRASQ | IGHV3-13 | IGHJ5 | IGKV1-39 | IGKJ1 | 2536 | ARVLYDSSGF | 3738 | QQSYEMP | Seth Zost et al., 2020 |
| 2462 | SISSYLNWYQQKPGKAPKLLIYAASSLK | (Human) | (Human) | (Human) | (Human) | | YNWFDP | | PWT | (https://www.nature.co |
| | SGVPSRFGGSGSGTDFTLTISSLQPEDF | | | | | | | | | m/articles/s41591-020- |
| | ATYYCQQSYEMPPWTFGQGTKVEIE | | | | | | | | | 0998-x) |
| CoV2- | SYELTQPPSVSVSPGQTARITCSGDALP | IGHV4-31 | IGHJ2 | IGLV3-25 | IGLJ1 | 2537 | ARDLGDGYN | 3739 | QSADSSGY | Seth Zost et al., 2020 |
| 2464 | NQYAYWYHQKPGQAPVVVIYKDSER | (Human) | (Human) | (Human) | (Human) | | LRVPAYFDL | | V | (https://www.nature.co |
| | PSGIPQRFSGSSSGTTVTLTISGVQAED | | | | | | | | | m/articles/s41591-020- |
| | EADYYCQSADSSGYVFGTGTKVTVL | | | | | | | | | 0998-x) |
| CoV2- | QSALTQPASVSGSPGQSITISCTGTSSD | IGHV4-4 | IGHJ4 | IGLV2-14 | IGLJ1 | 2538 | ARVDHVNVR | 3740 | SSYTSSS | Seth Zost et al., 2020 |
| 2465 | VGGYNYVSWYQQHPGKAPKLMIYDV | (Human) | (Human) | (Human) | (Human) | | DY | | IPYV | (https://www.nature.co |
| | SKRPSGVSNRFSGSKSGNTASLTISGLQ | | | | | | | | | m/articles/s41591-020- |
| | AEDEADYYCSSYTSSSIPYVFGTGTKVT | | | | | | | | | 0998-x) |
| | VL | | | | | | | | | |
| CoV2- | EIVLTQSPAILSLSPGERATLSCRASQSV | IGHV3-21 | IGHJ6 | IGKV3-11 | IGKJ1 | 2539 | ARVNGNSN | 3741 | QQRGNW | Seth Zost et al., 2020 |
| 2466 | SSYLAWYQQKPGQAPRLLIYDTSNRAT | (Human) | (Human) | (Human) | (Human) | | WNFGSYYYYY | | WT | (https://www.nature.co |
| | GIPARFSGSGSGTDFTLTISSLEPEDFAF | | | | | | MDV | | | m/articles/s41591-020- |
| | YYCQQRGNWWTFAQGTKVEIK | | | | | | | | | 0998-x) |
| CoV2- | SYELTQPPSVSVSPRQTARITCSGDALP | IGHV3-33 | IGHJ4 | IGLV3-10 | IGLJ3 | 2540 | AREGQMAAT | 3742 | YSTDTSGN | Seth Zost et al., 2020 |
| 2473 | KKYAYWYQQKSGQAPVLVIYEDSKRP | (Human) | (Human) | (Human) | (Human) | | TGIDY | | HWV | (https://www.nature.co |
| | SGIPERFSGSSSGTMATLTISGAQVED | | | | | | | | | m/articles/s41591-020- |
| | EADYYCYSTDTSGNHWVFGGGTKLTV | | | | | | | | | 0998-x) |
| | L | | | | | | | | | |
| CoV2- | SYELTQPPSVSVSPGQTARITCSGDALP | IGHV3-15 | IGHJ3 | IGLV3-10 | IGLJ3 | 2541 | TTLTYYYDSSA | 3743 | YSTDSSGN | Seth Zost et al., 2020 |
| 2474 | KKYAYWYQQKSGQAPVLVISEDSKRP | (Human) | (Human) | (Human) | (Human) | | YLNDAFDI | | HRV | (https://www.nature.co |
| | SGIPERFSGSSSGTVATLTISGAQVEDE | | | | | | | | | m/articles/s41591-020- |
| | ADYYCYSTDSSGNHRVFGGGTKLTVL | | | | | | | | | 0998-x) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV1, | | | SARS-CoV2 | S; non- | B-cells; SARS-CoV2 | 454 | EVQLVESGGGLVQPGRSLRLSCAASGFT | 1533 |
| 2478 | | SARS-CoV2 | | | | RBD | Human Patient | | FDDYAMHWVRQAPGKGLEWVSGISW | |
| | | | | | | | | | NSDNIGYADSVKGRFTISRDIAKNSLYLQ | |
| | | | | | | | | | MNSLRAEDTALYYCAKGIYYDIFMPLLD | |
| | | | | | | | | | WGRGTLVTVSS | |
| CoV2- | Ab | SARS-CoV2 | SARS- | SARS-CoV2 | | S; RBD | B-cells; SARS-CoV2 | 455 | QVQLVQSGAEVKKPGSSVKVSCKTSGD | 1534 |
| 2479 | | | CoV1 | | | | Human Patient | | TSSSYTVGWVRQAPGQGLEWMGRIIPI | |
| | | | | | | | | | LGIAYSAQKFQGRLTITADKSTSTSYMEL | |
| | | | | | | | | | SSLRSEDTAVYYCARGVVAATPGWFDP | |
| | | | | | | | | | WGQGTLVTVSS | |
| CoV2- | Ab | SARS-CoV1, | | | SARS-CoV2 | S; non- | B-cells; SARS-CoV2 | 456 | QVQLVQSGAEVKKPGSSVKVSCKASGG | 1535 |
| 2481 | | SARS-CoV2 | | | | RBD | Human Patient | | TFGSYVISWVRQAPGQGLQWMGGIIPI | |
| | | | | | | | | | FGKPNYAQKFQGRVTITADESTSTAYME | |
| | | | | | | | | | LSSLRSEDTAVYYCARGWFGELLKGTYW | |
| | | | | | | | | | FDPWGQGTLVTVSS | |
| CoV2- | Ab | SARS-CoV2 | SARS- | SARS-CoV2 | | S; RBD | B-cells; SARS-CoV2 | 457 | QVQLVQSGAEVKKPGSSVKVSCKASGG | 1536 |
| 2485 | | | CoV1 | (weak) | | | Human Patient | | TFSSYSITWVRQAPGQGLEWMGRIIPVL | |
| | | | | | | | | | GIANYAQKFQDRVTITADKSTSTAYMEL | |
| | | | | | | | | | SSLRSEDTAVYYCARVGVSGFKSGSNWY | |
| | | | | | | | | | FDLWGRGTLVTVSS | |
| CoV2- | Ab | SARS-CoV2 | SARS- | | SARS-CoV1, | S; non- | B-cells; SARS-CoV2 | 458 | EVQLVESGGGLVQPGGSLRLSCAVSGFT | 1537 |
| 2488 | | | CoV1 | | SARS-CoV2 | RBD | Human Patient | | FSSYWMHWVRQAPGKGLVWVSRINSD | |
| | | | | | | | | | GSSTSYADSVKGRFTISRDNAKNTLYLQ | |
| | | | | | | | | | MNSLRAEDTAVYYCAREVEQLAHMVDY | |
| | | | | | | | | | WGQGTLVTVSS | |
| CoV2- | Ab | SARS-CoV2 | SARS- | SARS-CoV2 | | S; NTD | B-cells; SARS-CoV2 | 459 | QLQLQESGPGLVKPSETLSLTCTVSGGSI | 1538 |
| 2489 | | | CoV1 | (weak) | | | Human Patient | | SSTTYYWGWIRQPPGKGLEWIASIYYSG | |
| | | | | | | | | | STYYNPSLKSRLTVSVDTSKNQFSLKLSSV | |
| | | | | | | | | | TAADTAVYYCARQWKWFGEAWYFDL | |
| | | | | | | | | | WGRGTLVTVSS | |
| CoV2- | Ab | SARS-CoV2 | SARS- | | SARS-CoV1, | S; NTD | B-cells; SARS-CoV2 | 460 | EVQLVESGGGLVQPGGSLRLSCAASGFT | 1539 |
| 2490 | | | CoV1 | | SARS-CoV2 | | Human Patient | | FSSYWMNWVRQAPGKGLEWVANINQ | |
| | | | | | | | | | DGGEKYYVDSVRGRFTISRDNAKNSLYL | |
| | | | | | | | | | QMNSLRAEDTAVYYCARDPYDLYGDYG | |
| | | | | | | | | | GTFDYWGQGTLVTVSS | |
| CoV2- | Ab | SARS-CoV2 | SARS- | | SARS-CoV1, | S; NTD | B-cells; SARS-CoV2 | 461 | QVQLVQSGAAVKKPGSSVKVSCKASGG | 1540 |
| 2495 | | | CoV1 | | SARS-CoV2 | | Human Patient | | TFSSYAISWVRQAPGQGLEWMGGIVPI | |
| | | | | | | | | | FGTANYAQKFQGRVTITADESTTTAYME | |
| | | | | | | | | | LNSLRSEDTAVYYCAREDYYGSGSLVDPY | |
| | | | | | | | | | YYYRMDVWGQGTTVTVSS | |
| CoV2- | Ab | SARS-CoV2 | SARS- | | SARS-CoV1, | S; non- | B-cells; SARS-CoV2 | 462 | QVQLVESGGGVVQPGRSLRLSCAASGF | 1541 |
| 2496 | | | CoV1 | | SARS-CoV2 | RBD | Human Patient | | TFSNYGMHWVRQAPGKGLEWVAVIW | |
| | | | | | | | | | YDGSNKYYADSVKGRFTISRDNSKNTLYL | |
| | | | | | | | | | QMNSLRAEDTAVYYCVRDLALFEVVIQ | |
| | | | | | | | | | QGVWGQGTLVTVSS | |
| CoV2- | Ab | SARS-CoV2 | SARS- | SARS-CoV2 | | S; RBD | B-cells; SARS-CoV2 | 463 | QLQLQESGPGLVKPSETLSLTCTVSGGSV | 1542 |
| 2499 | | | CoV1 | | | | Human Patient | | SSRSYYWGWIRQPPGKGLEWIGSIYYSG | |
| | | | | | | | | | STYYNPSLKSRVTISVDTSKNQFSLKLSSV | |
| | | | | | | | | | TAADTAVYYCARHTVDCGGDCFPNDAF | |
| | | | | | | | | | DIWGQGTMVTVSS | |
| CoV2- | Ab | SARS-CoV2 | SARS- | SARS-CoV2 | | S; RBD | B-cells; SARS-CoV2 | 464 | QVQLVQSGAEVKKPGASVKVSCKASGY | 1543 |
| 2504 | | | CoV1 | | | | Human Patient | | TFTDYYMHWVRQAPGQGLEWMGWI | |
| | | | | | | | | | NPNSRGTNYAQKFQGRVTMTRDTSIST | |
| | | | | | | | | | VYMELSRLTSDDTAVYYCARVVVLGYGR | |
| | | | | | | | | | PNNYYDGRNVWDYWGQGTLVTVSS | |
| CoV2- | Ab | SARS-CoV1, | | | SARS-CoV2 | S; non- | B-cells; SARS-CoV2 | 465 | QVQLVESGGGVVQPGRSLRLSCAASGFI | 1544 |
| 2509 | | SARS-CoV2 | | | | RBD | Human Patient | | FSTYAMHWVRQAPGKGLEWVAVISYD | |
| | | | | | | | | | GDNKYYADSVKGRFTISRDNSKNTLYLE | |
| | | | | | | | | | MNSLRAEDTAVYYCARPRGGSYQTCFD | |
| | | | | | | | | | YWGQGTLVTVSS | |
| CoV2- | Ab | SARS-CoV2 | SARS- | | SARS-CoV1, | S; RBD | B-cells; SARS-CoV2 | 466 | QVQLVQSGSELKKPGASVKVSCKASGYT | 1545 |
| 2510 | | | CoV1 | | SARS-CoV2 | | Human Patient | | FTSHTMNWVRQAPGQGLEWMGWIN | |
| | | | | | | | | | TNTGNPMYAQGFTGRFVFSLDTSGSTA | |
| | | | | | | | | | YLQISSLKAEDTAVYYCARWGPDYGDYA | |
| | | | | | | | | | SNDYWGQGTLVTVSS | |
| CoV2- | Ab | SARS-CoV1, | | SARS-CoV2 | | S; RBD | B-cells; SARS-CoV2 | 467 | EVQLVESGGGVVRPGGSLRLSCAASGFI | 1546 |
| 2514 | | SARS-CoV2 | | (weak) | | | Human Patient | | FDDYDMTWVRQAPGKGLEWVSGINW | |
| | | | | | | | | | NGGSTGYADSVKGRFTISRDNAKNSLYL | |
| | | | | | | | | | QMNSLRAEDTALYHCAVIMSPIPRYSGY | |
| | | | | | | | | | DWAGDAFDIWGQGTMVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | EIVLTQSPGTLSLSPGERATLSCRASQS | IGHV3-9 | IGHJ4 | IGKV3-20 | IGKJ2 | 2542 | AKGIYYDIFM | 3744 | HQYGTSPY | Seth Zost et al., 2020 |
| 2478 | VSSTFLAWYQQKPGQAPRLLIFGASSR | (Human) | (Human) | (Human) | (Human) | | PLLD | | T | (https://www.nature.co |
| | ATGIPDRFSGSGSGTDFTLTISRLEPED | | | | | | | | | m/articles/s41591-020- |
| | FAVYYCHQYGTSPYTFGQGTKLEIK | | | | | | | | | 0998-x) |
| CoV2- | EIVMTQSPATLSVSPGERVTLSCRASQ | IGHV1-69 | IGHJ5 | IGKV3-15 | IGKJ4 | 2543 | ARGVVAATP | 3745 | QQYNNFLT | Seth Zost et al., 2020 |
| 2479 | SVSSNLAWYQQKPGQAPRLLIYGAST | (Human) | (Human) | (Human) | (Human) | | GWFDP | | | (https://www.nature.co |
| | RATGIPARFSGGGSGTEFTLTISSLQSE | | | | | | | | | m/articles/s41591-020- |
| | DFAVYYCQQYNNFLTFGGGTKVEIK | | | | | | | | | 0998-x) |
| CoV2- | DIVMTQSPDSLAVSLGERATINCKSSQ | IGHV1-69 | IGHJ5 | IGKV4-1 | IGKJ2 | 2544 | ARGWFGELL | 3746 | QQYYSTPG | Seth Zost et al., 2020 |
| 2481 | SVLYSSNNKNYLAWYQQKPGQPPKLLI | (Human) | (Human) | (Human) | (Human) | | KGTYWFDP | | | (https://www.nature.co |
| | YWASTRESGVPDRFSGSGSGTDFTLTI | | | | | | | | | m/articles/s41591-020- |
| | SSLQAEDVAVYYCQQYYSTPGFGQGT | | | | | | | | | 0998-x) |
| | KLEIK | | | | | | | | | |
| CoV2- | QSVLTQPPSVSGAPGQRVTLSCTGSNS | IGHV1-69 | IGHJ2 | IGLV1-40 | IGLJ3 | 2545 | ARVGVSGFKS | 3747 | QSYDSSLS | Seth Zost et al., 2020 |
| 2485 | NIGAGYDVHWYQQLPGTAPKLLIYGN | (Human) | (Human) | (Human) | (Human) | | GSNWYFDL | | DSV | (https://www.nature.co |
| | SNRPSGVPDRFSGSKSGTSASLAITGL | | | | | | | | | m/articles/s41591-020- |
| | QAEDEADYYCQSYDSSLSDSVFGGGT | | | | | | | | | 0998-x) |
| | KVTVL | | | | | | | | | |
| CoV2- | SYELTQPPSVSVSPGQTARITCSGDALP | IGHV3-74 | IGHJ4 | IGLV3-25 | IGLJ3 | 2546 | AREVEQLAH | 3748 | QSADSSGT | Seth Zost et al., 2020 |
| 2488 | NQYAYWYQQKPGQAPVLVIYKDSERP | (Human) | (Human) | (Human) | (Human) | | MVDY | | SWV | (https://www.nature.co |
| | SGIPERFSGSSSGTTVTLTISGVQAEDE | | | | | | | | | m/articles/s41591-020- |
| | ADYYCQSADSSGTSWVFGGGTKLTVL | | | | | | | | | 0998-x) |
| CoV2- | EIVLTQSPGTLSLSPGERASLSCRASQS | IGHV4-39 | IGHJ2 | IGKV3-20 | IGKJ4 | 2547 | ARQWKWFG | 3749 | QQYGSSPF | Seth Zost et al., 2020 |
| 2489 | VSSSYLAWYQQKPGQAPRLLIYGASSR | (Human) | (Human) | (Human) | (Human) | | EAWYFDL | | T | (https://www.nature.co |
| | ATGIPDRFSGSGSGTDFTLTISRLEPED | | | | | | | | | m/articles/s41591-020- |
| | FAVYYCQQYGSSPFTFGGGTKVEIK | | | | | | | | | 0998-x) |
| CoV2- | DIQMTQSPSTLSASVGDRVTITCRASQ | IGHV3-7 | IGHJ4 | IGKV1-5 | IGKJ4 | 2548 | ARDPYDLYGD | 3750 | QQYNSYSL | Seth Zost et al., 2020 |
| 2490 | SISSWLAWYQQKPRKAPKLLIYKASTLE | (Human) | (Human) | (Human) | (Human) | | YGGTEDY | | T | (https://www.nature.co |
| | SGVPSRFSGSGSGTEFTLTISSLQPDDF | | | | | | | | | m/articles/s41591-020- |
| | ATYYCQQYNSYSLTFGGGTKVEIK | | | | | | | | | 0998-x) |
| CoV2- | EIVLTQSPATLSVSPGERATLSCRASQS | IGHV1-69 | IGHJ6 | IGKV3-15 | IGKJ1 | 2549 | AREDYYGSGS | 3751 | QQYNNW | Seth Zost et al., 2020 |
| 2495 | VSSNLAWYQQKPGQAPRLLIYGASTR | (Human) | (Human) | (Human) | (Human) | | LVDPYYYYRM | | WRT | (https://www.nature.co |
| | ATGIPARFSGSGSGTDFTLTISSLQSED | | | | | | DV | | | m/articles/s41591-020- |
| | FALYYCQQYNNWWRTFGQGTKVEIN | | | | | | | | | 0998-x) |
| CoV2- | SYELTQPPSVSVSPGQTARITCSGDALP | IGHV3-33 | IGHJ4 | IGLV3-10 | IGLJ3 | 2550 | VRDLALFEVVI | 3752 | YSTDSSGN | Seth Zost et al., 2020 |
| 2496 | KKYAYWYQQKSGQAPVLVIYEDSKRP | (Human) | (Human) | (Human) | (Human) | | QQGV | | HWV | (https://www.nature.co |
| | SGIPERFSGSSSGTMATLTISGAQVED | | | | | | | | | m/articles/s41591-020- |
| | EADYYCYSTDSSGNHWVFGGGTKLTV | | | | | | | | | 0998-x) |
| | L | | | | | | | | | |
| CoV2- | SSELTQDPAVSVALGQTVRITCQGDSL | IGHV4-39 | IGHJ3 | IGLV3-19 | IGLJ2 | 2551 | ARHTVDCGG | 3753 | NFRDSSGH | Seth Zost et al., 2020 |
| 2499 | RSYYASWYQQKPGQAPLLVIYGKNNR | (Human) | (Human) | (Human) | (Human) | | DCFPNDAFDI | | HPV | (https://www.nature.co |
| | PSGIPDRFSGSSSGNTPSLTITGAQAED | | | | | | | | | m/articles/s41591-020- |
| | EADYYCNFRDSSGHHPVFGEGTKLTVL | | | | | | | | | 0998-x) |
| CoV2- | QSVLTQPPSASGTPGQRVIISCSGSSSN | IGHV1-2 | IGHJ4 | IGLV1-44 | IGLJ2 | 2552 | ARVVVLGYGR | 3754 | AAWDDSL | Seth Zost et al., 2020 |
| 2504 | IGSNTVKWYHQLPGTAPKLLICSNNQR | (Human) | (Human) | (Human) | (Human) | | PNNYYDGRN | | NALV | (https://www.nature.co |
| | PSGVPDRFSGSKSDTSASLAISGLQSED | | | | | | VWDY | | | m/articles/s41591-020- |
| | EADYYCAAWDDSLNALVFGGGTKLTV | | | | | | | | | 0998-x) |
| | L | | | | | | | | | |
| CoV2- | EIVMTQSPATLSVSPGERATLSCRASQ | IGHV3-30- | IGHJ4 | IGKV3-15 | IGKJ1 | 2553 | ARPRGGSYQT | 3755 | QQYNNWP | Seth Zost et al., 2020 |
| 2509 | SVSSNLAWYQQKPGQAPRLLIYGAST | 3 | (Human) | (Human) | (Human) | | CFDY | | GT | (https://www.nature.co |
| | RATGIPARFSGSGSGTEFTLTISSLQSE | (Human) | | | | | | | | m/articles/s41591-020- |
| | DFAVYYCQQYNNWPGTFGQGTKVEI | | | | | | | | | 0998-x) |
| | K | | | | | | | | | |
| CoV2- | SYELTQPSSVSVSPGQTARITCSGDVLA | IGHV7-4- | IGHJ4 | IGLV3-27 | IGLJ2 | 2554 | ARWGPDYGD | 3756 | YSAADNN | Seth Zost et al., 2020 |
| 2510 | KNYARWFQQKPGQAPVLVIYKDSERP | 1 | (Human) | (Human) | (Human) | | YASNDY | | RV | (https://www.nature.co |
| | SGIPKRFSGSSSGTTVTLTISGAQVEDE | (Human) | | | | | | | | m/articles/s41591-020- |
| | ADYYCYSAADNNRVFGGGTKLTVL | | | | | | | | | 0998-x) |
| CoV2- | SSELTQDPAVSVALGQTVRITCQGDSL | IGHV3-20 | IGHJ3 | IGLV3-19 | IGLJ2 | 2555 | AVIMSPIPRYS | 3757 | NSRDSSGN | Seth Zost et al., 2020 |
| 2514 | RSYYASWYQQKPGQVPILVIYDKNNR | (Human) | (Human) | (Human) | (Human) | | GYDWAGDAF | | AVV | (https://www.nature.co |
| | PSGIPDRFSGSSSGNTASLTITGAQAED | | | | | | DI | | | m/articles/s41591-020- |
| | EADYYCNSRDSSGNAVVFGGGTKLTV | | | | | | | | | 0998-x) |
| | L | | | | | | | | | |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2 | Ab | SARS-CoV1, | | | SARS-CoV2 | S; non- | B-cells; SARS-CoV2 | 468 | QVQLVQSGSELKKPGASVKVSCKASGYT | 1547 |
| 2515 | | SARS-CoV2 | | | | RBD | Human Patient | | FTTYAMNWVRQAPGQGLEWMGWIN | |
| | | | | | | | | | TNTGNPTYAQDFTGRFVFSLDTSVSTAYL | |
| | | | | | | | | | QISSLKAEDTAVYYCARGLVGRIDPWGQ | |
| | | | | | | | | | GTLVTVSS | |
| CoV2- | Ab | SARS-CoV1, | | | SARS-CoV2 | S; RBD | B-cells; SARS-CoV2 | 469 | QVQLVQSGAEVKKPGASVKVSCKASGY | 1548 |
| 2516 | | SARS-CoV2 | | | | | Human Patient | | TFTNYYMHWVRQAPGQGLEWMGILN | |
| | | | | | | | | | PGAGSTSYAQKFQGRVTMTSDTSTNTV | |
| | | | | | | | | | YMQLSSLKSEDTAVYYCARDQQIVPHAD | |
| | | | | | | | | | GFDIWGQGTMVTVSS | |
| CoV2- | Ab | SARS-CoV1, | | | SARS-CoV2 | S; non- | B-cells; SARS-CoV2 | 470 | QVQLVQSGAEVRKPGSSVKVSCKASGG | 1549 |
| 2517 | | SARS-CoV2 | | | | RBD | Human Patient | | TFSSYAISWVRQAPGQGLEWMGGIIPV | |
| | | | | | | | | | FGTANYAQKFQGRVTITADKSTSTAFME | |
| | | | | | | | | | LNSLRSEDTAVYYCARIGSYPEYFQHWG | |
| | | | | | | | | | QGTLVTVSS | |
| CoV2 | Ab | SARS-CoV2 | SARS- | | SARS-CoV1, | S; RBD | B-cells; SARS-CoV2 | 471 | EVQLVESGGGLVQPGRSLRLSCAASGFT | 1550 |
| 2518 | | | CoV1 | | SARS-CoV2 | | Human Patient | | FDDYAMHWVRQAPGKGLEWVSGISW | |
| | | | | | | | | | NSGTIGYADSVKGRFIISRDNAKNSLYLQ | |
| | | | | | | | | | MNSLRPEDTALYYCAKDIIRQGEDGMD | |
| | | | | | | | | | VWGQGTTVTVSS | |
| CoV2- | Ab | SARS-CoV1, | | | SARS-CoV2 | S; non- | B-cells; SARS-CoV2 | 472 | EVQLVESGGGLVQPGGSLRLSCAASGFT | 1551 |
| 2520 | | SARS-CoV2 | | | | RBD | Human Patient | | FSSYWMSWVRQAPGKGLEWVANIKED | |
| | | | | | | | | | GSEKYYVDSVKGRFTISRDNAKNSLYLQ | |
| | | | | | | | | | MNSLRAEDTAVYYCARDVGGYSGYDLG | |
| | | | | | | | | | FDYYYYMDVWGKGTTVTVSS | |
| CoV2- | Ab | SARS-CoV2 | SARS- | | SARS-CoV1, | S; non- | B-cells; SARS-CoV2 | 473 | QVQLVESGGGVVQPGRSLRLACAASGF | 1552 |
| 2521 | | | CoV1 | | SARS-CoV2 | RBD | Human Patient | | TFSSYGMHWVRQAPGKGLEWVAVISY | |
| | | | | | | | | | DGNNKYYADSLKGRFTISRDDSKNTLYL | |
| | | | | | | | | | QMNSLRAEDTAVYYCAKDRTAVFLFFGL | |
| | | | | | | | | | GDAFDIWGQGTMVTVSS | |
| CoV2- | Ab | SARS-CoV1, | | | SARS-CoV2 | S; non- | B-cells; SARS-CoV2 | 474 | EVQLVQSGAEVKKPGESLKISCKGSGYSF | 1553 |
| 2524 | | SARS-CoV2 | | | | RBD | Human Patient | | TSHWIGWVRQMPGKGLEWMGIIYPG | |
| | | | | | | | | | DSDTRYSPSFQGQVTISADKSISTAYLQW | |
| | | | | | | | | | SSLKASDTAMYYCASALRERGVQLWSV | |
| | | | | | | | | | WGQGTLVTVSS | |
| CoV2- | Ab | SARS-CoV2 | SARS- | | SARS-CoV1, | S; NTD | B-cells; SARS-CoV2 | 475 | EVRLLESGGGLVQPGGSLRLSCAASGFT | 1554 |
| 2525 | | | CoV1 | | SARS-CoV2 | | Human Patient | | FTSYAMSWVRQAPGKGLQWVSTISVSG | |
| | | | | | | | | | GSTYYADSVKGRFTISRDNSKNTLYLQM | |
| | | | | | | | | | NSLRAEDTAVYYCAKDPASGIVGPTHFD | |
| | | | | | | | | | YWGQGTLVTVSS | |
| CoV2- | Ab | SARS-CoV2 | SARS- | | SARS-CoV1, | S; NTD | B-cells; SARS-CoV2 | 476 | QVQLVQSGAEVKKPGASVKVSCKVSGY | 1555 |
| 2526 | | | CoV1 | | SARS-CoV2 | | Human Patient | | TLTELSIHWVRQAPGKGLEWMGGFDP | |
| | | | | | | | | | EDAETIYAQQFQGRVTMTEDTSTDTAY | |
| | | | | | | | | | MELSSLKSEDTALYYCATGFAVFGRAAV | |
| | | | | | | | | | PYWGQGTLVTVSS | |
| CoV2- | Ab | SARS-CoV2 | SARS- | | SARS-CoV1, | S; RBD | B-cells; SARS-CoV2 | 477 | QVQLQESGPGLVKPSQTLSLTCTVSGGSI | 1556 |
| 2527 | | | CoV1 | | SARS-CoV2 | | Human Patient | | SSGDYYWSWIRQPPGKGLEWIGYIYYSG | |
| | | | | | | | | | STYYNPSLKSRVTISVDTSKNQVSLKLSSV | |
| | | | | | | | | | TAADTAVYYCARFRRSYGSGSYYNISFDY | |
| | | | | | | | | | WGQGTLVTVSS | |
| CoV2- | Ab | SARS-CoV2 | SARS- | | SARS-CoV1, | S; non- | B-cells; SARS-CoV2 | 478 | QVQLVQSGSELKKPGASVKVSCKASGYT | 1557 |
| 2529 | | | CoV1 | | SARS-CoV2 | RBD | Human Patient | | FTRHAMNWVRQAPGQGLEWMGWIN | |
| | | | | | | | | | TNTGNPTYAQGFTGRFVFSLDTSVSTAY | |
| | | | | | | | | | LQISSLKAEDTAVYYCVREYGSGHPLPIW | |
| | | | | | | | | | GQGTLVTVSS | |
| CoV2- | Ab | SARS-CoV2 | SARS- | SARS-CoV2 | | S; RBD | B-cells; SARS-CoV2 | 479 | QVQLQESGPGLVKPSETLSLTCSVSGGSI | 1558 |
| 2531 | | | CoV1 | | | | Human Patient | | RSYFWNWVRQPPGKGLEWIGYIYYSGS | |
| | | | | | | | | | TNYKPSFKSRVTISLDTSKNQISLKLSSVT | |
| | | | | | | | | | AADTAVYYCARATWLRDAFGIWGQGT | |
| | | | | | | | | | MVTVSS | |
| CoV2- | Ab | SARS-CoV2 | SARS- | | SARS-CoV1, | S; RBD | B-cells; SARS-CoV2 | 480 | QVQLVQSGAEVKKPGASVRVSCKAPGY | 1559 |
| 2532 | | | CoV1 | | SARS-CoV2 | | Human Patient | | TFTTYYIHWVRQAPGQGLEWMGIINPS | |
| | | | | | | | | | AGSTTYAQKFQGRVTMTRDTSTSTVYM | |
| | | | | | | | | | ELSSLRSEDTAVYYCARGFHVPAALRNW | |
| | | | | | | | | | FDPWGQGTLVTVSS | |
| CoV2- | Ab | SARS-CoV2 | SARS- | | SARS-CoV1, | S; NTD | B-cells; SARS-CoV2 | 481 | QVQLQESGPGLVKPSQTLSLTCTVSGGSI | 1560 |
| 2533 | | | CoV1 | | SARS-CoV2 | | Human Patient | | SSGGYYWSWIRQHPGKGLEWIGYIYYS | |
| | | | | | | | | | GSTYYNPSLKSRVTISVDTSKNQFSLKLTS | |
| | | | | | | | | | VTAADTAVYYCAREANDSGSFYNGPFDY | |
| | | | | | | | | | WGQGTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2 | DIQMTQSPSSLSASVGDRVTITCQASQ | IGHV7-4- | IGHJ5 | IGKV1-33 | IGKJ3 | 2556 | ARGLVGRIDP | 3758 | QQYDNLL | Seth Zost et al., 2020 |
| 2515 | DISNSLSWYQQKPGKAPKLLIYDASNL | 1 | (Human) | (Human) | (Human) | | | | LFT | (https://www.nature.co |
| | KTGVPSRFSGSGSGTDFTFTISSLQPED | (Human) | | | | | | | | m/articles/s41591-020- |
| | IATYYCQQYDNLLLFTFGPGTKVDIK | | | | | | | | | 0998-x) |
| CoV2- | EIVLTQSPATLSLSPGERATLSCRASQS | IGHV1-46 | IGHJ3 | IGKV3-11 | IGKJ1 | 2557 | ARDQQIVPH | 3759 | QQRSNWP | Seth Zost et al., 2020 |
| 2516 | VSSYLAWYQQKPGQAPRLLIYDASNR | (Human) | (Human) | (Human) | (Human) | | ADGFDI | | PRTWT | (https://www.nature.co |
| | ATGIPARFSGSGSGTDFTLTISSLEPEDF | | | | | | | | | m/articles/s41591-020- |
| | AVYYCQQRSNWPPRTWTFGQGTKVE | | | | | | | | | 0998-x) |
| | IK | | | | | | | | | |
| CoV2- | EIVLTQSPATLSLSPGERATLSCRASQS | IGHV1-69 | IGHJ1 | IGKV3-11 | IGKJ4 | 2558 | ARIGSYPEY | 3760 | HYRSNWP | Seth Zost et al., 2020 |
| 2517 | VSSFLAWYQQKPGQAPRLLIYDASNR | (Human) | (Human) | (Human) | (Human) | | FQH | | PVLT | (https://www.nature.co |
| | ATGIPARFSGSGSGTDFTLTISSLEPEDF | | | | | | | | | m/articles/s41591-020- |
| | AVYYCHYRSNWPPVLTFGGGTKVEIE | | | | | | | | | 0998-x) |
| CoV2 | SSELTQDPAVSVALGQTVRITCQGDSL | IGHV3-9 | IGHJ6 | IGLV3-19 | IGLJ2 | 2559 | AKDIIRQGED | 3761 | NSRDSS | Seth Zost et al., 2020 |
| 2518 | RSYYASWYQQKPGQVPILVIYDKNNR | (Human) | (Human) | (Human) | (Human) | | GMDV | | GNAVV | (https://www.nature.co |
| | PSGIPDRFSGSSSGNTASLTITGAQAED | | | | | | | | | m/articles/s41591-020- |
| | EADYYCNSRDSSGNAVVFGGGTKLTV | | | | | | | | | 0998-x) |
| | A | | | | | | | | | |
| CoV2- | SSELTQDPAVSVALGQTVRITCQGDSL | IGHV3-7 | IGHJ6 | IGLV3-19 | IGLJ3 | 2560 | ARDVGGYSG | 3762 | NSRDSS | Seth Zost et al., 2020 |
| 2520 | RSYYANWYQQKPGQAPVVVIFGKNN | (Human) | (Human) | (Human) | (Human) | | YDLGFDYYYY | | GYIWGWM | (https://www.nature.co |
| | RPSGIPDRFSGSNSGNTASLTITGAQA | | | | | | MDV | | | m/articles/s41591-020- |
| | EDEADYYCNSRDSSGYIWGWMFGGG | | | | | | | | | 0998-x) |
| | TKLTVL | | | | | | | | | |
| CoV2- | SYVLTQPPSVSVAPGKTARITCGGNNI | IGHV3-30 | IGHJ3 | IGLV3-21 | IGLJ3 | 2561 | AKDRTAVFLF | 3763 | QVWDSSS | Seth Zost et al., 2020 |
| 2521 | GSKSVHWYQQKPGQAPVVVVYDDSD | (Human) | (Human) | (Human) | (Human) | | FGLGDAFDI | | DHWV | (https://www.nature.co |
| | RPSGLPERFSGSNSGNTATLTISRVEA | | | | | | | | | m/articles/s41591-020- |
| | GDEADYYCQVWDSSSDHWVFGGGT | | | | | | | | | 0998-x) |
| | KLTVL | | | | | | | | | |
| CoV2- | QSVLTQPPSVSGAPGQRVTISCTGSSS | IGHV5-51 | IGHJ4 | IGLV1-40 | IGLJ3 | 2562 | ASALRERGVQ | 3764 | QSYDSS | Seth Zost et al., 2020 |
| 2524 | NIGAGYDVHWYQQLPGTAPKLLIFINS | (Human) | (Human) | (Human) | (Human) | | LWSV | | LGAL | (https://www.nature.co |
| | NRPSGVPDRFSGSKSGTSASLAITGLQ | | | | | | | | | m/articles/s41591-020- |
| | AEDEADYYCQSYDSSLGALFGGGTKLT | | | | | | | | | 0998-x) |
| | VL | | | | | | | | | |
| CoV2- | QLVLTQSPSASASLGASVKLTCTLSSGH | IGHV3-23 | IGHJ4 | IGLV4-69 | IGLJ3 | 2563 | AKDPASGIVG | 3765 | QSWDTGI | Seth Zost et al., 2020 |
| 2525 | TTYAIAWHQQQPEKGPRYLMKLNSD | (Human) | (Human) | (Human) | (Human) | | PTHFDY | | GV | (https://www.nature.co |
| | GSHTRGDGIPDRFSGSSSGAERYLTISS | | | | | | | | | m/articles/s41591-020- |
| | LQSEDEADYYCQSWDTGIGVFGGGTK | | | | | | | | | 0998-x) |
| | LTVL | | | | | | | | | |
| CoV2- | SYELTQPPSVSVSPGQTASITCFGDKLG | IGHV1-24 | IGHJ4 | IGLV3-1 | IGLJ2 | 2564 | ATGFAVFGRA | 3766 | QAWDSST | Seth Zost et al., 2020 |
| 2526 | DKYACWFQQKPGQSPVLIIYQGAKRP | (Human) | (Human) | (Human) | (Human) | | AVPY | | VV | (https://www.nature.co |
| | SGIPERFSGSNSGNTATLTISGTQAMD | | | | | | | | | m/articles/s41591-020- |
| | EADYYCQAWDSSTVVFGGGTKLTVL | | | | | | | | | 0998-x) |
| CoV2- | QSALTQPASVSGSPGQSITISCTGTSSD | IGHV4-30- | IGHJ4 | IGLV2-23 | IGLJ2 | 2565 | ARFRRSYGSG | 3767 | CSYVGS | Seth Zost et al., 2020 |
| 2527 | VGSYNLVSWYQQHPGKAPKLMIYEVS | 4 | (Human) | (Human) | (Human) | | SYYNISFDY | | STYVV | (https://www.nature.co |
| | KRPSGVSNRFSGSKSGNTASLTISGLQ | (Human) | | | | | | | | m/articles/s41591-020- |
| | AEDEADYYCCSYVGSSTYVVFGGGTKL | | | | | | | | | 0998-x) |
| | TVL | | | | | | | | | |
| CoV2- | SYELTQPPSVSVSPGQTARITCSADALS | IGHV7-4- | IGHJ4 | IGLV3-25 | IGLJ3 | 2566 | VREYGSGHPL | 3768 | QSADSS | Seth Zost et al., 2020 |
| 2529 | KQYAYWYQQKPGQAPVVVIYKDSERP | 1 | (Human) | (Human) | (Human) | | PI | | SHWV | (https://www.nature.co |
| | SGIPERFSGSSSGTTVTLTISGVQAEDE | (Human) | | | | | | | | m/articles/s41591-020- |
| | ADYYCQSADSSSHWVFGGGTKLTVL | | | | | | | | | 0998-x) |
| CoV2- | NFMLTQPHSVSESPGKTVTISCTGNSG | IGHV4-59 | IGHJ3 | IGLV6-57 | IGLJ2 | 2567 | ARATWLRDA | 3769 | QSFDSG | Seth Zost et al., 2020 |
| 2531 | SIASNYVQWYQQRPGSAPTTVIYEDS | (Human) | (Human) | (Human) | (Human) | | FGI | | NVV | (https://www.nature.co |
| | QRPSGVPDRFSGSIDSSSNSASLTISGL | | | | | | | | | m/articles/s41591-020- |
| | KTEDEADYYCQSFDSGNVVFGGGTKL | | | | | | | | | 0998-x) |
| | TVL | | | | | | | | | |
| CoV2- | QSVLTQPPSASGTPGQRVTISCSGSSS | IGHV1-46 | IGHJ5 | IGLV1-44 | IGLJ2 | 2568 | ARGFHVPAA | 3770 | VAWDDSR | Seth Zost et al., 2020 |
| 2532 | NIGSNTVNWYQQLPGTAPKLLIYNNN | (Human) | (Human) | (Human) | (Human) | | LRNWFDP | | NGLV | (https://www.nature.co |
| | QRPSGVPDRLSGSKSGTSASLAISGLQ | | | | | | | | | m/articles/s41591-020- |
| | SEDEADYYCVAWDDSRNGLVFGGGT | | | | | | | | | 0998-x) |
| | KLTVL | | | | | | | | | |
| CoV2- | DIVMTQSPDSLAVSLGERATINCKSSQ | IGHV4-31 | IGHJ4 | IGKV4-1 | IGKJ2 | 2569 | AREANDSGS | 3771 | QQYYST | Seth Zost et al., 2020 |
| 2533 | SVLYSSNNKNYLAWYQQKPGQPPKLLI | (Human) | (Human) | (Human) | (Human) | | FYNGPFDY | | RT | (https://www.nature.co |
| | YWASTRESGVPDRFSGSGSGTDFTLTI | | | | | | | | | m/articles/s41591-020- |
| | SSLQAEDVAVYYCQQYYSTRTFGQGT | | | | | | | | | 0998-x) |
| | KLEIK | | | | | | | | | |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV1, | | | SARS-CoV2 | S; non- | B-cells; SARS-CoV2 | 482 | QVQLQESGPGLVKPSQTLSLTCTVSGGSI | 1561 |
| 2536 | | SARS-CoV2 | | | | RBD | Human Patient | | SSGSYYWSWIRQPAGKGLEWIGRIFTSG | |
| | | | | | | | | | STNYNPSLKSRVTISVDTSKNQFSLKLSSV | |
| | | | | | | | | | TAADTAVYYCARGGLLWFGGAGNYMD | |
| | | | | | | | | | VWGKGTTVTVSS | |
| CoV2- | Ab | SARS-CoV2 | SARS- | SARS-CoV2 | | S; RBD | B-cells; SARS-CoV2 | 483 | QVQLAQSGAEVKKPGASVKVSCKAGGY | 1562 |
| 2539 | | | CoV1 | | | | Human Patient | | TFTSYDINWVRQATGQGLEWMGWMN | |
| | | | | | | | | | SNSGNAGYAQKFQGRVTMTRDTSISTA | |
| | | | | | | | | | YMELSSLRSEDTAVYYCARMRTGWPTH | |
| | | | | | | | | | GRPDDFWGRGTLVTVSS | |
| CoV2- | Ab | SARS-CoV2 | SARS- | | SARS-CoV1, | S; non- | B-cells; SARS-CoV2 | 484 | QVQIQQWGAGVLKPSETLSLTCAVYGG | 1563 |
| 2545 | | | CoV1 | | SARS-CoV2 | RBD | Human Patient | | SFSGHYWSWIRQPPGKGLEWIGEINHS | |
| | | | | | | | | | GSTKYNPSLKSRVTISVDTSKNQFSLKLSS | |
| | | | | | | | | | VTAADTAVYYCARGPPVTTFFVFSLLFDP | |
| | | | | | | | | | WGQGTLVTVSS | |
| CoV2- | Ab | SARS-CoV2 | SARS- | | SARS-CoV1, | S; non- | B-cells; SARS-CoV2 | 485 | QVQLVESGGGVVQPGRSLRLSCAASGF | 1564 |
| 2546 | | | CoV1 | | SARS-CoV2 | RBD | Human Patient | | TFSSYGMHWVRQAPGKGLEWVAVISY | |
| | | | | | | | | | DGRNKYYADSVKGRFTISRDNSKNTLYL | |
| | | | | | | | | | QMSSLRAEDTAVYYCAKEGEWELRGNA | |
| | | | | | | | | | LDIWGQGTMVTVSS | |
| CoV2- | Ab | SARS-CoV2 | SARS- | | SARS-CoV1, | S; non- | B-cells; SARS-CoV2 | 486 | QVQLVESGGGVVQPGRSLRLSCAASGF | 1565 |
| 2549 | | | CoV1 | | SARS-CoV2 | RBD | Human Patient | | TFSSYGMHWVRQAPDKGLEWVAVIWY | |
| | | | | | | | | | DGSNKYYADSVKGRFTISRDNSKNTLYL | |
| | | | | | | | | | QMNSLRAEDTAVYYCAREGQWPNQAF | |
| | | | | | | | | | DIWGQGTMVTVSS | |
| CoV2- | Ab | SARS-CoV1, | | | SARS-CoV2 | S; non- | B-cells; SARS-CoV2 | 487 | QVQLQESGPGLVKPSETLSLTCTVSGGSI | 1566 |
| 2551 | | SARS-CoV2 | | | | RBD | Human Patient | | SSSSYYWGWIRQPPGKGLEWIGSIYYSG | |
| | | | | | | | | | SSYYSPSLKSRVTISADTSKNQFSLNLRSV | |
| | | | | | | | | | TAADTAVYYCASGPPYMATFSYYFDYW | |
| | | | | | | | | | GQGTLVTVSS | |
| CoV2- | Ab | SARS-CoV1, | | SARS-CoV2 | | S; RBD | B-cells; SARS-CoV2 | 488 | QVQLVESGGGLVKPGGSLRLSCAASGFT | 1567 |
| 2552 | | SARS-CoV2 | | (weak) | | | Human Patient | | FSDYYMSWIRQAPGKGLECVSYISSSGST | |
| | | | | | | | | | IYYADSVKGRFTISRDNAKNSLYLQMNSL | |
| | | | | | | | | | RAEDTAVYYCARDPIRDGVWGLNENDY | |
| | | | | | | | | | WGQGTLVTVSS | |
| CoV2- | Ab | SARS-CoV2 | SARS- | | SARS-CoV1, | S; non- | B-cells; SARS-CoV2 | 489 | QVQLVESGGGVVQPGRSLRLSCAASGFI | 1568 |
| 2553 | | | CoV1 | | SARS-CoV2 | RBD | Human Patient | | FSSYAMHWVRQAPGKGLEWVAVISYD | |
| | | | | | | | | | GSNKYYADSVKGRFTISRDNSKNTLYLQ | |
| | | | | | | | | | MNSLRAEDTAVYYCARSPPASYYNPSTG | |
| | | | | | | | | | YFDYWGQGTLVTVSS | |
| CoV2- | Ab | SARS-CoV1, | | | SARS-CoV2 | S; RBD | B-cells; SARS-CoV2 | 490 | QVQLVQSGAEVKKPGASVKVSCKASGY | 1569 |
| 2554 | | SARS-CoV2 | | | | | Human Patient | | TFTSHYMHWVRQAPGQGLEWMGIINP | |
| | | | | | | | | | SGGSTSYAQKFQGRVTMTSDTSTSTVY | |
| | | | | | | | | | MELSSLRSEDTAMYYCARDVFWVPAAS | |
| | | | | | | | | | SFDYWGQGTLVTVSS | |
| CoV2- | Ab | SARS-CoV2 | SARS- | | SARS-CoV1, | S; NTD | B-cells; SARS-CoV2 | 491 | QVQLVESGGGVVQPGRSLRLSCAASGF | 1570 |
| 2558 | | | CoV1 | | SARS-CoV2 | | Human Patient | | TFSSYAMHWVRQAPGKGLEWVAVISY | |
| | | | | | | | | | DGSNKYYADSVKGRFTISRDNSKNTLYL | |
| | | | | | | | | | QMNSLRVEDTAVYYCARDQEWFRELFL | |
| | | | | | | | | | FDYWGQGTLVTVSS | |
| CoV2- | Ab | SARS-CoV2 | SARS- | SARS-CoV2 | | S; RBD | B-cells; SARS-CoV2 | 492 | QVQLAQSGAEVKKPGASVKVSCKAAGY | 1571 |
| 2562 | | | CoV1 | | | | Human Patient | | TFTSYDINWVRQATGQGLEWMGWMN | |
| | | | | | | | | | SNSGNAGYAQKFQGRVTMTRDTSTSTA | |
| | | | | | | | | | YMELSSLTSDDTAVYYCARMRTGWPTH | |
| | | | | | | | | | GRPDDFWGRGTLVTVSS | |
| CoV2- | Ab | SARS-CoV2 | SARS- | | SARS-CoV1, | S; NTD | B-cells; SARS-CoV2 | 493 | QVQLQQWGAGLLKPSETLSLTCAVYGG | 1572 |
| 2563 | | | CoV1 | | SARS-CoV2 | | Human Patient | | SFSGYYWSWIRQPPGKGLEWIGEINHS | |
| | | | | | | | | | GSSNYNPSLKSRVTISVDTSKNQFSLKLSS | |
| | | | | | | | | | VTAADTAVYYCARGWGWGAVAGRAEY | |
| | | | | | | | | | YFDYWGQGTLVTVSS | |
| CoV2- | Ab | SARS-CoV1, | | | SARS-CoV2 | S; non- | B-cells; SARS-CoV2 | 494 | QVQLVESGGGVVQPGRSLRLSCAASGF | 1573 |
| 2564 | | SARS-CoV2 | | | | RBD | Human Patient | | TFSSYAMHWVRQAPGKGLEWVAVISY | |
| | | | | | | | | | DGYNKYYADSVKGRFTISRDNSKNTLYL | |
| | | | | | | | | | QMNSLRAEDTAVYYCARAQGGNYYYG | |
| | | | | | | | | | MDVWGQGTTVTVSS | |
| CoV2- | Ab | SARS-CoV1, | | | SARS-CoV2 | S; non- | B-cells; SARS-CoV2 | 495 | QVQLQESGPGLVKPSQTLSLTCTVSGGSI | 1574 |
| 2565 | | SARS-CoV2 | | | | RBD | Human Patient | | SSGSYYWSWIRQPAGKGLEWIGRVYIYS | |
| | | | | | | | | | SGSTNYNPSLKSRVTISVDTSKNQFSLKLS | |
| | | | | | | | | | SVTAADTAVYYCARGAASFDYWGQGTL | |
| | | | | | | | | | VTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | DIVMTQSPDSLAVSLGERATINCKSSQ | IGHV4-61 | IGHJ6 | IGKV4-1 | IGKJ2 | 2570 | ARGGLLWFG | 3772 | QQYYSTPP | Seth Zost et al., 2020 |
| 2536 | SVLYSSNNKNYLAWYQQKPGQPPKLLI | (Human) | (Human) | (Human) | (Human) | | GAGNYMDV | | YT | (https://www.nature.co |
| | YWASTRESGVPDRFSGSGSGTDFTLTI | | | | | | | | | m/articles/s41591-020- |
| | SSLQAEDVAVYYCQQYYSTPPYTFGQG | | | | | | | | | 0998-x) |
| | TKVEIK | | | | | | | | | |
| CoV2- | QSVLTQPPSASGTPGQRVTISCSGSNS | IGHV1-8 | IGHJ4 | IGLV1-44 | IGLJ3 | 2571 | ARMRTGWPT | 3773 | LAWDDSL | Seth Zost et al., 2020 |
| 2539 | NIGSYTVNWYQQFPGTAPKLLIYDNN | (Human) | (Human) | (Human) | (Human) | | HGRPDDF | | NGLV | (https://www.nature.co |
| | QRTSGVPDRFSGSKSGTSASLAISGLQ | | | | | | | | | m/articles/s41591-020- |
| | SEDEASYYCLAWDDSLNGLVFGGGTK | | | | | | | | | 0998-x) |
| | LTVL | | | | | | | | | |
| CoV2- | QSVLTQPPSASGTPGQRVTISCSGSSS | IGHV4-34 | IGHJ5 | IGLV1-47 | IGLJ3 | 2572 | ARGPPVTTFF | 3774 | AAWDASL | Seth Zost et al., 2020 |
| 2545 | NIGSNYVYWYQQLPGTAPKLLIYSSNQ | (Human) | (Human) | (Human) | (Human) | | VFSLLFDP | | SGWV | (https://www.nature.co |
| | RPSGVPDRFSGSKSGTSASLAISGLRSE | | | | | | | | | m/articles/s41591-020- |
| | DEADYYCAAWDASLSGWVFGGGTKL | | | | | | | | | 0998-x) |
| | TVL | | | | | | | | | |
| CoV2- | SYELTQPPSVSVSPGQTARITCSADALP | IGHV3-30 | IGHJ3 | IGLV3-25 | IGLJ3 | 2573 | AKEGEWELR | 3775 | QSVDGSGS | Seth Zost et al., 2020 |
| 2546 | KHYAYWYQQRPGQAPVLVIYKDIERP | (Human) | (Human) | (Human) | (Human) | | GNALDI | | SVV | (https://www.nature.co |
| | SGIPERFSGSSSGTTVTLTITGAQAEDE | | | | | | | | | m/articles/s41591-020- |
| | ADYYCQSVDGSGSSVVFGGGTKLAVL | | | | | | | | | 0998-x) |
| CoV2- | SYELTQPPSVSVSPGQTASITCSGDKLG | IGHV3-33 | IGHJ3 | IGLV3-1 | IGLJ2 | 2574 | AREGQWPN | 3776 | QAWDSST | Seth Zost et al., 2020 |
| 2549 | DKYACWYQQKPGQSPVLVIHQDSQR | (Human) | (Human) | (Human) | (Human) | | QAFDI | | HVV | (https://www.nature.co |
| | PSGIPERFSGSNSGNTATLTISGTQAM | | | | | | | | | m/articles/s41591-020- |
| | DEADYYCQAWDSSTHVVFGGGTKLT | | | | | | | | | 0998-x) |
| | VL | | | | | | | | | |
| CoV2- | QSALTQPASVSGSPGQSITISCTGTSSD | IGHV4-39 | IGHJ4 | IGLV2-14 | IGLJ1 | 2575 | ASGPPYMATF | 3777 | SSYTSSSTP | Seth Zost et al., 2020 |
| 2551 | VGGYNYVSWFQHHPDKAPRLMIYDV | (Human) | (Human) | (Human) | (Human) | | SYYFDY | | FV | (https://www.nature.co |
| | SKRPSGVSNRFSGSKSGNTASLTISGLQ | | | | | | | | | m/articles/s41591-020- |
| | AEDEADYYCSSYTSSSTPFVFGTGTKVT | | | | | | | | | 0998-x) |
| | VL | | | | | | | | | |
| CoV2- | QPVLTQSSSASASLGSSVKLTCTLSSGH | IGHV3-11 | IGHJ4 | IGLV4-60 | IGLJ3 | 2576 | ARDPIRDGV | 3778 | ETWDSNT | Seth Zost et al., 2020 |
| 2552 | SSYIIAWHQQQPGKAPRYLMKLEGSG | (Human) | (Human) | (Human) | (Human) | | WGLNENDY | | RV | (https://www.nature.co |
| | SYNKGSGVPDRFSGSSSGADCYLTISN | | | | | | | | | m/articles/s41591-020- |
| | LQSEDEADYYCETWDSNTRVFAGGTK | | | | | | | | | 0998-x) |
| | LTVL | | | | | | | | | |
| CoV2- | DIQMTQSPSSLSASVGDRVTITCRASQ | IGHV3-30 | IGHJ4 | IGKV1-39 | IGKJ2 | 2577 | ARSPPASYYN | 3779 | QQSYSTP | Seth Zost et al., 2020 |
| 2553 | SISSYLNWYQQKPGKAPKLLIYAASSLQ | (Human) | (Human) | (Human) | (Human) | | PSTGYFDY | | MHT | (https://www.nature.co |
| | SGVPSRFSGSGSGTDFTLTISSLQPEDF | | | | | | | | | m/articles/s41591-020- |
| | ATFYCQQSYSTPMHTFGQGTKLEIK | | | | | | | | | 0998-x) |
| CoV2- | DIQLTQSPSFLSASVGDRVTITCRASQG | IGHV1-46 | IGHJ4 | IGKV1-9 | IGKJ1 | 2578 | ARDVFWVPA | 3780 | QQLNSYLG | Seth Zost et al., 2020 |
| 2554 | ISSYLAWYQQKPGKAPKLLIYAASTLQS | (Human) | (Human) | (Human) | (Human) | | ASSFDY | | T | (https://www.nature.co |
| | GVPSRFSGSGSGTEFTLTISSLQPEDFA | | | | | | | | | m/articles/s41591-020- |
| | TYYCQQLNSYLGTFGQGTKVEIK | | | | | | | | | 0998-x) |
| CoV2- | DIQMTQSPSSVSASVGDRVTITCRASQ | IGHV3-30- | IGHJ4 | IGKV1-12 | IGKJ3 | 2579 | ARDQEWFRE | 3781 | QQANSFPP | Seth Zost et al., 2020 |
| 2558 | GISSWLAWYQQKPGKAPKLLIYDASSL | 3 | (Human) | (Human) | (Human) | | LFLFDY | | T | (https://www.nature.co |
| | QSGVPSRFSGSGSGTDFTLTISSLQPED | (Human) | | | | | | | | m/articles/s41591-020- |
| | FATYYCQQANSFPPTFGPGTKVDIK | | | | | | | | | 0998-x) |
| CoV2- | QSVLTQPPSASGTPGQRVTISCSGSNS | IGHV1-8 | IGHJ4 | IGLV1-44 | IGLJ2 | 2580 | ARMRTGWPT | 3782 | LVWDDSL | Seth Zost et al., 2020 |
| 2562 | NIGSYTVNWYQQFPGTAPKLLIYDNN | (Human) | (Human) | (Human) | (Human) | | HGRPDDF | | NGLV | (https://www.nature.co |
| | QRTSGVPDRFSGSKSGTSASLAISGLQ | | | | | | | | | m/articles/s41591-020- |
| | SEDEANYYCLVWDDSLNGLVFGGGTK | | | | | | | | | 0998-x) |
| | LTVL | | | | | | | | | |
| CoV2- | QSALTQPASVSGSPGQSITISCTGTSSD | IGHV4-34 | IGHJ4 | IGLV2-23 | IGLJ1 | 2581 | ARGWGWGA | 3783 | CSYAGSST | Seth Zost et al., 2020 |
| 2563 | VGSYNLVSWYQQHPGKAPKLMIYEVS | (Human) | (Human) | (Human) | (Human) | | VAGRAEYYFD | | WG | (https://www.nature.co |
| | KRPSGVSNRFSGSKSGNTASLTISGLQ | | | | | | Y | | | m/articles/s41591-020- |
| | AEDEADYYCCSYAGSSTWGFGTGTKV | | | | | | | | | 0998-x) |
| | TVL | | | | | | | | | |
| CoV2- | SYVLTQPPSVSVAPGKTARITCGGNNI | IGHV3-30- | IGHJ6 | IGLV3-21 | IGLJ2 | 2582 | ARAQGGNYY | 3784 | QVWDSSS | Seth Zost et al., 2020 |
| 2564 | GTKGVHWYQQKPGQAPVLVVYDDS | 3 | (Human) | (Human) | (Human) | | YGMDV | | DHHVV | (https://www.nature.co |
| | DRPSGIPGRFSGSNSGNTATLTISRVEA | (Human) | | | | | | | | m/articles/s41591-020- |
| | GDEADYFCQVWDSSSDHHVVFGGGT | | | | | | | | | 0998-x) |
| | KLTVL | | | | | | | | | |
| CoV2- | DIVMTQSPLSLPVTPGEPASISCRSSQS | IGHV4-61 | IGHJ4 | IGKV2-28 | IGKJ4 | 2583 | ARGAASFDY | 3785 | MQALQTP | Seth Zost et al., 2020 |
| 2565 | LLHSDGYTYLDWYLQKPGQSPQLLIYL | (Human) | (Human) | (Human) | (Human) | | | | LT | (https://www.nature.co |
| | GSKRASGVPDRFSGSGSGTDFTLKISR | | | | | | | | | m/articles/s41591-020- |
| | VEAEDVGVYYCMQALQTPLTFGGGTK | | | | | | | | | 0998-x) |
| | VEIK | | | | | | | | | |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV2 | SARS- | | SARS-CoV1, | S; non- | B-cells; SARS-CoV2 | 496 | QLQLQESGPGLVKPSETLSLTCTVSGGSI | 1575 |
| 2570 | | | CoV1 | | SARS-CoV2 | RBD | Human Patient | | SSSSNYWGWIRQPPGKGLEWIGSIYYSG | |
| | | | | | | | | | STYYNPSLKSRVTISVDTSKNQFSLKLSSV | |
| | | | | | | | | | TAADTAVYYCARDPRVVVTARMYNWF | |
| | | | | | | | | | DPWGQGTLVTVSS | |
| CoV2- | Ab | SARS-CoV2 | SARS- | | SARS-CoV1, | S; NTD | B-cells; SARS-CoV2 | 497 | QVQLQESGPGLVKPSETLSLTCTVSGGSL | 1576 |
| 2571 | | | CoV1 | | SARS-CoV2 | | Human Patient | | SSYYWSWIRQPPGKGLEWIGYIYDSGG | |
| | | | | | | | | | ASRSTNYNPSLKSRVTISVDTSRNQLSLKL | |
| | | | | | | | | | SSVTAADTAVYYCARDQRQFQLLGRFG | |
| | | | | | | | | | WFDPWGQGTLVTVSS | |
| CoV2- | Ab | SARS-CoV2 | SARS- | | SARS-CoV1, | S; non- | B-cells; SARS-CoV2 | 498 | QLQLQESGPGLVKPSETLSLTCTVSGGSI | 1577 |
| 2574 | | | CoV1 | | SARS-CoV2 | RBD | Human Patient | | SSTTYYWGWIRQPPGKGLEWIASIYYSG | |
| | | | | | | | | | STYYNPSLKSRLTVSVDTSKNQFSLKLSSV | |
| | | | | | | | | | TAADTAVYYCARQWKWFGEAWYFDL | |
| | | | | | | | | | WGRGTLVTVSS | |
| CoV2- | Ab | SARS-CoV2 | SARS- | | SARS-CoV1, | S; non- | B-cells; SARS-CoV2 | 499 | QVQLVQSGSELKKPGASVKVSCKASGYT | 1578 |
| 2582 | | | CoV1 | | SARS-CoV2 | RBD | Human Patient | | FTTYAMNWVRQAPGQGLEWMGWIN | |
| | | | | | | | | | TNTGNPTYAQGFTGRFVFSLDTSVNTAF | |
| | | | | | | | | | LHIGSLKAEDTAVYYCARDQDSGYPTYYY | |
| | | | | | | | | | YYMDVWGKGTTVTVSS | |
| CoV2- | Ab | SARS-CoV1, | | | SARS-CoV2 | S; non- | B-cells; SARS-CoV2 | 500 | QVQLVQSGAEVKKPGASVKVSCKASGY | 1579 |
| 2583 | | SARS-CoV2 | | | | RBC | Human Patient | | TFTSYDMNWVRQATGQGLEWMGWM | |
| | | | | | | | | | NPNSGNTGYAQKFQGRVTMTRNTSIST | |
| | | | | | | | | | AYMELSSLRSEDTAVYYCARGGIYYLVRG | |
| | | | | | | | | | FIIGYYGMDVWGQGTTVTVSS | |
| CoV2- | Ab | SARS-CoV2 | SARS- | | SARS-CoV1, | S; non- | B-cells; SARS-CoV2 | 501 | EVALVESGGGLVKPGGSLRLSCAASGFIF | 1580 |
| 2584 | | | CoV1 | | SARS-CoV2 | RBD | Human Patient | | SNAWMTWVRQAPGKGLEWVGRIKSK | |
| | | | | | | | | | SEGGTPEYAAPVKGRFIISRDDSTNSLHL | |
| | | | | | | | | | QMNYLRIEDTAVYYCTTGGYSSYAASDY | |
| | | | | | | | | | WGQGTLVTVSS | |
| CoV2- | Ab | SARS-CoV2 | SARS- | | SARS-CoV1, | S; RBD | B-cells; SARS-CoV2 | 502 | EVQLVESGGGLVQPGGSLRLSCAASGFT | 1581 |
| 2585 | | | CoV1 | | SARS-CoV2 | | Human Patient | | FSSYSMNWVRQAPGKGLEWVSYISSRS | |
| | | | | | | | | | STIKYADSVKGRFTISRDNAKNSLYLQM | |
| | | | | | | | | | NSLRDEDTAVYYCARVDYYGSGSVYWYF | |
| | | | | | | | | | DLWGRGTLVTVSS | |
| CoV2- | Ab | SARS-CoV2 | SARS- | | SARS-CoV1, | S; non- | B-cells; SARS-CoV2 | 503 | EVQLVESGGGLVQPGRSLRLSCAASGFT | 1582 |
| 2586 | | | CoV1 | | SARS-CoV2 | RBD | Human Patient | | FDDYAMHWVRQAPGKGLEWVSGISW | |
| | | | | | | | | | NSGSIAYADSVKGRFTISRDNAKNSLYLQ | |
| | | | | | | | | | MNSLRAEDTALYFCAKVGWELSIDAFDL | |
| | | | | | | | | | WGQGTMVTVSS | |
| CoV2- | Ab | SARS-CoV1, | | | SARS-CoV2 | S; non- | B-cells; SARS-CoV2 | 504 | QITFKESGPTLVKPTETLTLTCTFSGFSVS | 1583 |
| 2587 | | SARS-CoV2 | | | | RBC | Human Patient | | TSGEGVGWIRQPPGKALEWLAVIYWD | |
| | | | | | | | | | DDKRYSPSLKSRLTITRDTSKNQVVLTMT | |
| | | | | | | | | | NMDPVDTATYYCAHRLWFRDAFDIWG | |
| | | | | | | | | | QGTTVTVSS | |
| CoV2- | Ab | SARS-CoV2 | SARS- | SARS-CoV2 | | S; RBD | B-cells; SARS-CoV2 | 505 | QVTLRESGPALVKPTQTLTLTCTFSGFSLS | 1584 |
| 2589 | | | CoV1 | (weak) | | | Human Patient | | TSGMCVSWIRQPPGKALEWLARIDWD | |
| | | | | | | | | | DDKYYSTSLETRLTISKDTSKNQVVLTMT | |
| | | | | | | | | | NMDPVDTATYYCARIQYQLNGMDVW | |
| | | | | | | | | | GQGTTVTVSS | |
| CoV2- | Ab | SARS-CoV1, | | | SARS-CoV2 | S; non- | B-cells; SARS-CoV2 | 506 | QVQLVQSGAEVKKPGASVKVSCKASGY | 1585 |
| 2590 | | SARS-CoV2 | | | | RBD | Human Patient | | TFSSYDINWVRQATGQGLEWMGWVN | |
| | | | | | | | | | PNSGHTGYAQKFQGRVTMTRNTSVSTA | |
| | | | | | | | | | YMELSSLRSEDTAVFYCARGRVGYVGSG | |
| | | | | | | | | | SRGYYYYYDMDVWGQGTTVTVSS | |
| CoV2- | Ab | SARS-CoV1, | | | SARS-CoV2 | S; non- | B-cells; SARS-CoV2 | 507 | EVQLVQSGAEVKKPGESLKISCKGSGYSF | 1586 |
| 2602 | | SARS-CoV2 | | | | RBD | Human Patient | | TSYWIGWVRQMPGKGLEWMGIIYPGD | |
| | | | | | | | | | SDTRYSPSFQGQVTISADKSISTAYLQWS | |
| | | | | | | | | | SLKASDTAMYYCARPDYSSGWFSYWYF | |
| | | | | | | | | | DLWGRGTLVTVSS | |
| CoV2- | Ab | SARS-CoV1, | | | SARS-CoV2 | S; non- | B-cells; SARS-CoV2 | 508 | QVQLVQSGSALKKPGASVKVSCKASGYT | 1587 |
| 2610 | | SARS-CoV2 | | | | RBD | Human Patient | | FTSYAMNWVRQAPGQGLEWMGWINT | |
| | | | | | | | | | NTGNPTYAQGFTGRFVFSLDTSVSTAYL | |
| | | | | | | | | | QISSLKAEDTAVYYCARGRSYGLSLGYW | |
| | | | | | | | | | GQGTLVTVSS | |
| CoV2- | Ab | SARS-CoV2 | SARS- | | SARS-CoV1, | S; non- | B-cells; SARS-CoV2 | 509 | QVQLVESGGGVVQPGRSLRVSCAASGF | 1588 |
| 2611 | | | CoV1 | | SARS-CoV2 | RBD | Human Patient | | TFSSHGMHWVRQAPGKGLEWVSVIWY | |
| | | | | | | | | | DGSNKYYADSVKGRFTISRDNSKNTLSL | |
| | | | | | | | | | QMNSLRAEDTAVYYCARESADISSRLDY | |
| | | | | | | | | | WGRGTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | EIVMTQSPGTLSVSPGERATLSCRASQ | IGHV4-39 | IGHJ5 | IGKV3-15 | IGKJ2 | 2584 | ARDPRVVVTA | 3786 | QQYNNWP | Seth Zost et al., 2020 |
| 2570 | SVSSNLAWYQQKPGQAPRLLIYGAST | (Human) | (Human) | (Human) | (Human) | | RMYNWFDP | | PMYT | (https://www.nature.co |
| | RATGVPARFSGSGSGTEFTLTISSLQSE | | | | | | | | | m/articles/s41591-020- |
| | DFAVYYCQQYNNWPPMYTFGQGTKL | | | | | | | | | 0998-x) |
| | EIK | | | | | | | | | |
| CoV2- | EIVMTQSPATLSVSPGERATLSCRASQ | (Human) | IGHJ5 | IGKV3-15 | IGKJ1 | 2585 | ARDQRQFQL | 3787 | QQYNNWP | Seth Zost et al., 2020 |
| 2571 | SVSSNLAWYQQKPGQAPRLLIYGAST | IGHV4-59 | (Human) | (Human) | (Human) | | LGRFGWFDP | | RT | (https://www.nature.co |
| | RATGIPARFSGSGSGTEFTLTISSLQSE | | | | | | | | | m/articles/s41591-020- |
| | DFAVYYCQQYNNWPRTFGQGTKVEIK | | | | | | | | | 0998-x) |
| CoV2- | QPVVTQSPSASASLGASVKLTCTLDSG | IGHV4-39 | IGHJ2 | IGLV4-69 | IGLJ2 | 2586 | ARQWKWFG | 3788 | QTWGT | Seth Zost et al., 2020 |
| 2574 | HRSYAIAWHQQRPEKGPRFLMRITTD | (Human) | (Human) | (Human) | (Human) | | EAWYFDL | | | (https://www.nature.co |
| | GRHTKGDGIPDRFSGSGSGTERYLTISS | | | | | | | | | m/articles/s41591-020- |
| | LQSEDEADYYCQTWGTFGGGTRLTVL | | | | | | | | | 0998-x) |
| CoV2- | DIVMTQTPLSLSVTPGQPASISCKSSQS | IGHV7-4- | IGHJ6 | IGKV2D-29 | IGKJ4 | 2587 | ARDQDSGYP | 3789 | MQSIQPPL | Seth Zost et al., 2020 |
| 2582 | LLHSDGKTYLYWYLQKPGQSPQLLIYE | 1 | (Human) | (Human) | (Human) | | TYYYYYMDV | | T | (https://www.nature.co |
| | VSNRFSGVPDRFSGSGSGTDFTLKISR | (Human) | | | | | | | | m/articles/s41591-020- |
| | VEAEDVGVYYCMQSIQPPLTFGGGTK | | | | | | | | | 0998-x) |
| | VEIK | | | | | | | | | |
| CoV2- | DIVMTQSPDSLAVSLGERATINCKSSQ | IGHV1-8 | IGHJ6 | IGKV4-1 | IGKJ4 | 2588 | ARGGIYYLVR | 3790 | QQYYSTPL | Seth Zost et al., 2020 |
| 2583 | SVLYSSNNKNYLAWYQQKPGQPPKLLI | (Human) | (Human) | (Human) | (Human) | | GFIIGYYGMD | | T | (https://www.nature.co |
| | YWASTRESGVPDRFSGSGSGTDFTLTI | | | | | | V | | | m/articles/s41591-020- |
| | SSLQAEDVAVYYCQQYYSTPLTFGGGT | | | | | | | | | 0998-x) |
| | KVEIK | | | | | | | | | |
| CoV2- | QSALTQPASVSGSPGQLITIFCNGSISD | IGHV3-15 | IGHJ4 | IGLV2-14 | IGLJ2 | 2589 | TTGGYSSYAA | 3791 | SSFTSRGA | Seth Zost et al., 2020 |
| 2584 | VGGWNYVSWYQQHPDKAPKMMIY | (Human) | (Human) | (Human) | (Human) | | SDY | | LVL | (https://www.nature.co |
| | DVRHRPSGVSSRFSGSKSGNTASLTIS | | | | | | | | | m/articles/s41591-020- |
| | GLQAEDEGDYYCSSFTSRGALVLFGG | | | | | | | | | 0998-x) |
| | GTKLTVL | | | | | | | | | |
| CoV2- | QSALTQPRSVSGSPGQSVTISCTGTSS | IGHV3-48 | IGHJ2 | IGLV2-11 | IGLJ2 | 2590 | ARVDYYGSGS | 3792 | CSYAGIWV | Seth Zost et al., 2020 |
| 2585 | DVGGYNYVSWYQQHPGKAPKLMIYD | (Human) | (Human) | (Human) | (Human) | | VYWYFDL | | | (https://www.nature.co |
| | VSKRPSGVPDRFSGSKSGNTASLTISGL | | | | | | | | | m/articles/s41591-020- |
| | QAEDEADYYCCSYAGIWVFGGGTKLT | | | | | | | | | 0998-x) |
| | VL | | | | | | | | | |
| CoV2- | EIVLTQSPGTLSLSPGERATLSCRASQS | IGHV3-9 | IGHJ3 | IGKV3-20 | IGKJ2 | 2591 | AKVGWELSID | 3793 | QHYGSSRS | Seth Zost et al., 2020 |
| 2586 | VSSSYLGWYHQKPGQAPRLLIYGASSR | (Human) | (Human) | (Human) | (Human) | | AFDL | | T | (https://www.nature.co |
| | ATGIPDRFSGSGSGTDFTLTISRLEPED | | | | | | | | | m/articles/s41591-020- |
| | FAVYYCQHYGSSRSTFGQGTKLEIK | | | | | | | | | 0998-x) |
| CoV2- | DIQMTQSPSSLSASVGDRVTITCRARQ | IGHV2-5 | IGHJ3 | IGKV1-39 | IGKJ4 | 2592 | AHRLWFRDA | 3794 | QQSYSTPT | Seth Zost et al., 2020 |
| 2587 | SISNYLNWYQQKPGKAPKLLIYAASSL | (Human) | (Human) | (Human) | (Human) | | FDI | | | (https://www.nature.co |
| | HSGVPSRFSGSGSGTDFTLTISSLQPED | | | | | | | | | m/articles/s41591-020- |
| | FATYYCQQSYSTPTFGGGTKVEIK | | | | | | | | | 0998-x) |
| CoV2- | DIQMTQSPSSLSASVGDRVTITCRASQ | IGHV2-70 | IGHJ6 | IGKV1-39 | IGKJ2 | 2593 | ARIQYQLNG | 3795 | QQSYSTPY | Seth Zost et al., 2020 |
| 2589 | SISSYLNWYQQKPGKAPKLLIYAASSLH | (Human) | (Human) | (Human) | (Human) | | MDV | | T | (https://www.nature.co |
| | SGVPSRFSGSGSGTDFTLTISSLQPEDF | | | | | | | | | m/articles/s41591-020- |
| | ATYYCQQSYSTPYTFGQGTKLEIK | | | | | | | | | 0998-x) |
| CoV2- | SSELTQDPAVSVALGQTVRITCQGDSL | IGHV1-8 | IGHJ6 | IGLV3-19 | IGLJ2 | 2594 | ARGRVGYVG | 3796 | NSRDSSGN | Seth Zost et al., 2020 |
| 2590 | RSYYASWYQQKPRQAPVLIISGNNNR | (Human) | (Human) | (Human) | (Human) | | SGSRGYYYYY | | HLRV | (https://www.nature.co |
| | PSGIPDRFSGSSSGNTASLTITGAQAED | | | | | | DMDV | | | m/articles/s41591-020- |
| | EADYYCNSRDSSGNHLRVFGGGTKLT | | | | | | | | | 0998-x) |
| | VL | | | | | | | | | |
| CoV2- | EIVLTQSPGTLSLSPGERATLSCRASQS | IGHV5-51 | IGHJ2 | IGKV3-20 | IGKJ5 | 2595 | ARPDYSSGW | 3797 | QQYGRSPI | Seth Zost et al., 2020 |
| 2602 | VSSNFLAWYQQKPGQAPRLLIYGASS | (Human) | (Human) | (Human) | (Human) | | FSYWYFDL | | T | (https://www.nature.co |
| | RATGIPDRFSGSGSGTDFTLTISRLEPE | | | | | | | | | m/articles/s41591-020- |
| | DFAVYYCQQYGRSPITFGQGTRLEIK | | | | | | | | | 0998-x) |
| CoV2- | DIQMTQSPSSLSASVGDRVTITCQASQ | IGHV7-4- | IGHJ4 | IGKV1-33 | IGKJ3 | 2596 | ARGRSYGLSL | 3798 | QQYDNLL | Seth Zost et al., 2020 |
| 2610 | DISNYLNWYQQKPGKAPKLLIYAASNL | 1 | (Human) | (Human) | (Human) | | GY | | QFT | (https://www.nature.co |
| | ETGVPSRFSGSGSGTDFTFTISSLQPED | (Human) | | | | | | | | m/articles/s41591-020- |
| | IATYYCQQYDNLLQFTFGPGTKVDIK | | | | | | | | | 0998-x) |
| CoV2- | SYELTQPPSVSVSPGQTARITCSGDALP | IGHV3-33 | IGHJ4 | IGLV3-10 | IGLJ1 | 2597 | ARESADISS | 3799 | YSTDSSGN | Seth Zost et al., 2020 |
| 2611 | TKYAYWYQQKSGQAPVLVIYDDSKRP | (Human) | (Human) | (Human) | (Human) | | RLDY | | V | (https://www.nature.co |
| | SGIPERFSGSSSGTMATLTISGAQVED | | | | | | | | | m/articles/s41591-020- |
| | EADYYCYSTDSSGNVFGTGTKVTVL | | | | | | | | | 0998-x) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV1, | | | SARS-CoV2 | S; non- | B-cells; SARS-CoV2 | 510 | QITFKESGPTLVKPTETLTLTCTFSGFSVS | 1589 |
| 2614 | | SARS-CoV2 | | | | RBD | Human Patient | | TSGEGVGWIRQPPGKALEWLAVIYWD | |
| | | | | | | | | | DDKRYSPSLKSRLTITRDTSKNQVVLTMT | |
| | | | | | | | | | NMDPVDTATYYCAHRLWFRDAFDIWG | |
| | | | | | | | | | QGTTVTVSS | |
| CoV2- | Ab | SARS-CoV1, | | | SARS-CoV2 | S; non- | B-cells; SARS-CoV2 | 511 | EVQLVESGGGLVQPGGSLRLSCAASGFI | 1590 |
| 2616 | | SARS-CoV2 | | | | RBD | Human Patient | | LSDHYMDWVRQAPGKGLEWVGRTRN | |
| | | | | | | | | | KANSYTTEYAASVKGRFTISRDDSKNSLY | |
| | | | | | | | | | LQMNSLKTEDTAVYYCASVITFGGVIVRS | |
| | | | | | | | | | YWGQGTLVTVSS | |
| CoV2- | Ab | SARS-CoV1, | | | SARS-CoV2 | S; non- | B-cells; SARS-CoV2 | 512 | QVQLVQSGAEVKKPGSSVKVSCKASGG | 1591 |
| 2617 | | SARS-CoV2 | | | | RBD | Human Patient | | TFSSYAISWVRQAPGQGLEWMGGIIPV | |
| | | | | | | | | | FGTTNYAQKLQGRVTISADESTSTAYME | |
| | | | | | | | | | VSSLRSEDTAVYYCARVSGYGDYGAYSD | |
| | | | | | | | | | YWGQGTLVTVSS | |
| CoV2- | Ab | SARS-CoV2 | SARS- | | SARS-CoV1, | S; RBD | B-cells; SARS-CoV2 | 513 | EVQLVESGGGLVQPGGSLRLSCVASEFT | 1592 |
| 2618 | | | CoV1 | | SARS-CoV2 | | Human Patient | | FSSYWMSWFRQAPGKGLEWVSGINW | |
| | | | | | | | | | NGGSTGYADSVKGRFTISRDNAKNSLYL | |
| | | | | | | | | | QMNSLKTEDTAVYYCASVITFGGVIVRSY | |
| | | | | | | | | | WGQGTLVTVSS | |
| CoV2- | Ab | SARS-CoV1, | | | SARS-CoV2 | S; non- | B-cells; SARS-CoV2 | 514 | EVQLLESGGGLVQPGGSLRLSCAASGFT | 1593 |
| 2619 | | SARS-CoV2 | | | | RBD | Human Patient | | FNNYAMSWVRQAPGKGLEWVSAIGGS | |
| | | | | | | | | | GGSTYYADSVKGRFTVSRDNSENTLYLQ | |
| | | | | | | | | | MSSLRAEDTAVYYCARVEGDWLLGGPY | |
| | | | | | | | | | YHYYGMDVWGQGTTVTVSS | |
| CoV2- | Ab | SARS-CoV1, | | | SARS-CoV2 | S; non- | B-cells; SARS-CoV2 | 515 | QVQLVESGGGVVQPGRSLRLSCAASGF | 1594 |
| 2620 | | SARS-CoV2 | | | | RBD | Human Patient | | TFSSYAMHWVRQAPGKGLEWVAGISY | |
| | | | | | | | | | DGSNKYYADSVKGRFTISRDNSKNTLYL | |
| | | | | | | | | | QMNSLRAEDTAVYYCARADTMVRGTYF | |
| | | | | | | | | | EYWGQGTLVTVSS | |
| CoV2- | Ab | SARS-CoV2 | SARS- | | SARS-CoV1, | S; non- | B-cells; SARS-CoV2 | 516 | QVQLVESGGGVVQPGRSLRLSCAASGF | 1595 |
| 2621 | | | CoV1 | | SARS-CoV2 | RBD | Human Patient | | TFSSYLMHWVRQAPGKGLEWVAVIWA | |
| | | | | | | | | | NGNRYYADSVKGRFTISRDISKNTLYLQ | |
| | | | | | | | | | MNSLRAEDTAMYYCARDYCNGVTCNS | |
| | | | | | | | | | NYWGQGTLVTVSS | |
| CoV2- | Ab | SARS-CoV2 | SARS- | | SARS-CoV1, | S; NTD | B-cells; SARS-CoV2 | 517 | QVQLQESGPGLVKPSGTLSLTCAVSGGS | 1596 |
| 2622 | | | CoV1 | | SARS-CoV2 | | Human Patient | | ISSSNWWSWVRQPPGKGLEWIGEIYHS | |
| | | | | | | | | | GSTNYNPSLKSRVTISVDKSKNQFSLKLSS | |
| | | | | | | | | | VTAADTAVYYCARGWYFDYWGQGTLV | |
| | | | | | | | | | TVSA | |
| CoV2- | Ab | SARS-CoV2 | SARS- | | SARS-CoV1, | S; non- | B-cells; SARS-CoV2 | 518 | QVQLVQSGAEVKKPGSSVKVSCKASGG | 1597 |
| 2624 | | | CoV1 | | SARS-CoV2 | RBD | Human Patient | | TFHNYAISWVRQAPGQGLEWMGGFIPI | |
| | | | | | | | | | LGTTNYAQKFQGRVTITADESTSTAYME | |
| | | | | | | | | | LSSLRSEDTAVYYCARVEGEGVDSYYYG | |
| | | | | | | | | | MDVWGQGTTVTVSS | |
| CoV2- | Ab | SARS-CoV2 | SARS- | SARS-CoV2 | | S; RBD | B-cells; SARS-CoV2 | 519 | EVQLVESGGGLVQPGRSLRLSCAASGFT | 1598 |
| 2628 | | | CoV1 | (weak) | | | Human Patient | | FDDYAMHWVRQAPGKGLEWVSGISW | |
| | | | | | | | | | NSGTIGYADSVKGRFIISRDNAKNSLYLQ | |
| | | | | | | | | | MNSLRPEDTALYYCAKDIIRQGEDGMD | |
| | | | | | | | | | VWGQGTTVTVSS | |
| CoV2- | Ab | SARS-CoV2 | SARS- | | SARS-CoV1, | S; RBD | B-cells; SARS-CoV2 | 520 | QLQVVQSGPGLVKPSETLSLTCTVSGDP | 1599 |
| 263 | | | CoV1 | | SARS-CoV2 | | Human Patient | | VINTNYYWGWIRQPPGKGLEWIGTLSY | |
| | | | | | | | | | SGGTHYNPSLSSRVTIAVDSSKKRFSLTLR | |
| | | | | | | | | | SVTAADTAIYYCARHPVDGYNYGYSDL | |
| | | | | | | | | | WGQGTLVTVSS | |
| CoV2- | Ab | SARS-CoV2 | SARS- | | SARS-CoV1, | S; RBD | B-cells; SARS-CoV2 | 521 | QVQLVQSGAEVKKPGASVKVSCKASGY | 1600 |
| 2632 | | | Cov1 | | SARS-CoV2 | | Human Patient | | TFTDYYMHWVRQAPGQGLEWMGWI | |
| | | | | | | | | | NPNSRGTNYAQKFQGRVTMTRDTSIST | |
| | | | | | | | | | VYMELSRLTSDDTAVYYCARVVVLGYGR | |
| | | | | | | | | | PNNYYDGRNVWDYWGQGTLVTVSS | |
| CoV2- | Ab | SARS-CoV1, | | | SARS-CoV2 | S; non- | B-cells; SARS-CoV2 | 522 | QVQLVESGGGVVQPGRSLRLSCAASGF | 1601 |
| 2639 | | SARS-CoV2 | | | | RBD | Human Patient | | TFSSYAMHWVRQAPGKGLEWVAVISY | |
| | | | | | | | | | DEINKYYADSVKGRFTISRDNSKTTLDLQ | |
| | | | | | | | | | MNSLRAEDTAVYYCARAGGGSYRGPFD | |
| | | | | | | | | | YWGQGTLVTVSS | |
| CoV2- | Ab | SARS-CoV1, | | | SARS-CoV2 | S; non- | B-cells; SARS-CoV2 | 523 | QVQLVESGGGVVQPGRSLRLSCAASGF | 1602 |
| 2641 | | SARS-CoV2 | | | | RBD | Human Patient | | TFSTYAMHWVRQAPGKGLEWVTVISYD | |
| | | | | | | | | | GSNKYYADSVKGRFTISRDNSKNTLYLQ | |
| | | | | | | | | | MNSLRAEDTAVYYCAKSYNGNYYDAFDI | |
| | | | | | | | | | WGQGTMVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | EIVMTQSPATLSVSAGERATLSCRASQ | IGHV2-5 | IGHJ3 | IGKV3-15 | IGKJ4 | 2598 | AHRLWFRDA | 3800 | QQYNSYSL | Seth Zost et al., 2020 |
| 2614 | SISSNLAWYHQKPGQAPRLLIYGASTR | (Human) | (Human) | (Human) | (Human) | | FDI | | T | (https://www.nature.co |
| | ATGIPARFSGSGSGTEFTLTISSLQSEDF | | | | | | | | | m/articles/s41591-020- |
| | AVYYCQQYNSYSLTFGGGTKVEIK | | | | | | | | | 0998-x) |
| CoV2- | EIVLTQSPGTLSLSPGERATLSCRASQS | IGHV3-72 | IGHJ4 | IGKV3-20 | IGKJ3 | 2599 | ASVITFGGVIV | 3801 | QYYGSSPF | Seth Zost et al., 2020 |
| 2616 | VSSSYLAWYQQKPGQAPRLLIYGASSR | (Human) | (Human) | (Human) | (Human) | | RSY | | G | (https://www.nature.co |
| | ATGIPDRFSGSGSGTDFTLTISRLEPED | | | | | | | | | m/articles/s41591-020- |
| | FAVYYCQYYGSSPFGFGPGTKVDIK | | | | | | | | | 0998-x) |
| CoV2- | EIVLTQSPATLSLSPGERATLSCRASQS | IGHV1-69 | IGHJ4 | IGKV3-11 | IGKJ4 | 2600 | ARVSGYGDY | 3802 | QQRSNWP | Seth Zost et al., 2020 |
| 2617 | VSRFLAWYQQKPGQAPRLLIYDASNR | (Human) | (Human) | (Human) | (Human) | | GAYSDY | | PRLT | (https://www.nature.co |
| | ATGIPARFSGSGSGTDFTLTISSLEPEDF | | | | | | | | | m/articles/s41591-020- |
| | AVYYCQQRSNWPPRLTFGGGTKVEIK | | | | | | | | | 0998-x) |
| CoV2- | QSVLTQPPSASGTPGQRVTISCSGSSS | IGHV3-20 | IGHJ4 | IGLV1-44 | IGLJ2 | 2601 | ASVITFGGVIV | 3803 | AVWDDSL | Seth Zost et al., 2020 |
| 2618 | NIGSNTVNWYQQLPGTAPKLLIYSNN | (Human) | (Human) | (Human) | (Human) | | RSY | | NGVV | (https://www.nature.co |
| | QRPSGVPDRFSGSKSGTSASLAISGLQ | | | | | | | | | m/articles/s41591-020- |
| | SEDENNYYCAVWDDSLNGVVFGGGT | | | | | | | | | 0998-x) |
| | KLTVL | | | | | | | | | |
| CoV2- | QSVLTQPPSASGTPGQRVTISCSGSSS | IGHV3-23 | IGHJ6 | IGLV1-47 | IGLJ2 | 2602 | ARVEGDWLL | 3804 | LVWDDSL | Seth Zost et al., 2020 |
| 2619 | NIGSNYVYWYQQLPGTAPKLLISRNN | (Human) | (Human) | (Human) | (Human) | | GGPYYHYYG | | NGLV | (https://www.nature.co |
| | QRPSGVPDRFSGSKSGTSASLAISGLQ | | | | | | MDV | | | m/articles/s41591-020- |
| | SEDEANYYCLVWDDSLNGLVFGGGTK | | | | | | | | | 0998-x) |
| | LTVL | | | | | | | | | |
| CoV2- | QSALTQPASVSGSPGQSITISCTGTSSD | IGHV3-30- | IGHJ4 | IGLV2-14 | IGLJ2 | 2603 | ARADTMVRG | 3805 | SSYTSSRAV | Seth Zost et al., 2020 |
| 2620 | VGGYNYVSWYQQHPGKAPKLMIYDV | 3 | (Human) | (Human) | (Human) | | TYFEY | | L | (https://www.nature.co |
| | SNRPSGVSNRFSGSKSGNTASLTISGL | (Human) | | | | | | | | m/articles/s41591-020- |
| | QAEDEADYCCSSYTSSRAVLFGGGTKL | | | | | | | | | 0998-x) |
| | TVL | | | | | | | | | |
| CoV2- | DFVMTQSPGSLAVSLGERATINCRSSQ | IGHV3-33 | IGHJ4 | IGKV4-1 | IGKJ1 | 2604 | ARDYCNGVT | 3806 | QQYYSSH | Seth Zost et al., 2020 |
| 2621 | SVLDNSSNKNHLAWHQQKPGQPPKL | (Human) | (Human) | (Human) | (Human) | | CNSNY | | WT | (https://www.nature.co |
| | LIYWASTRESGVPDRFSGSGSGTDFTL | | | | | | | | | m/articles/s41591-020- |
| | TISSLQAEDVAVYYCQQYYSSHWTFG | | | | | | | | | 0998-x) |
| | QGTKVEIK | | | | | | | | | |
| CoV2- | QSALTQPASVSGSPGQSITISCTGTSSD | IGHV4-4 | IGHJ4 | IGLV2-23 | IGLJ3 | 2605 | ARGWYFDY | 3807 | YSYAGSST | Seth Zost et al., 2020 |
| 2622 | VGSYNLVSWYQQHPGKAPKLMIYEVS | (Human) | (Human) | (Human) | (Human) | | | | WV | (https://www.nature.co |
| | KRPSGVSNRFSGSKSGNTASLTISGLQ | | | | | | | | | m/articles/s41591-020- |
| | AEDEADYYCYSYAGSSTWVFGGGTKL | | | | | | | | | 0998-x) |
| | TVL | | | | | | | | | |
| CoV2- | SYELTQPPSVSVSPGQTARITCSADALP | IGHV1-69 | IGHJ6 | IGLV3-25 | IGLJ2 | 2606 | ARVEGEGVD | 3808 | QSTDSSGS | Seth Zost et al., 2020 |
| 2624 | KQYAYWYQQKPGQAPVLVIYKDSERP | (Human) | (Human) | (Human) | (Human) | | SYYYGMDV | | YVV | (https://www.nature.co |
| | SGIPERFSGSSSGTTVTLTISGVQAEDE | | | | | | | | | m/articles/s41591-020- |
| | ADYYCQSTDSSGSYVVFGGGTKLTVL | | | | | | | | | 0998-x) |
| CoV2- | DIQMTQSPSSLSASVGDRVTITCRASQ | IGHV3-9 | IGHJ6 | IGKV1-39 | IGKJ2 | 2607 | AKDIIRQGED | 3809 | HQSYFTPQ | Seth Zost et al., 2020 |
| 2628 | NIASYLNWYQQKPGKAPKLLIYAASSL | (Human) | (Human) | (Human) | (Human) | | GMDV | | T | (https://www.nature.co |
| | QSGVPSRFSGSGSGTDFTLTISSLQPEE | | | | | | | | | m/articles/s41591-020- |
| | FATYYCHQSYFTPQTFGQGTKLEIK | | | | | | | | | 0998-x) |
| CoV2- | EIVLTQSPATLALSVGERATLSCVASQR | IGHV4-39 | IGHJ5 | IGKV3D-20 | IGKJ3 | 2608 | ARHPVDGYN | 3810 | QQYGNSP | Seth Zost et al., 2020 |
| 263 | VSSDYIAWYQKKPGLAPRLLVYHGSA | (Human) | (Human) | (Human) | (Human) | | YGYSDL | | FT | (https://www.nature.co |
| | WATGSSARFSGSGSGTAFTLTISSLEPE | | | | | | | | | m/articles/s41591-020- |
| | DFAVYYCQQYGNSPFTFGPGTKVEFK | | | | | | | | | 0998-x) |
| CoV2- | EIVLTQSPGTLSLSPGERATLSCRASQS | IGHV1-2 | IGHJ4 | IGKV3-20 | IGKJ2 | 2609 | ARVVVLGYGR | 3811 | QQYVEPPF | Seth Zost et al., 2020 |
| 2632 | VSSSYLAWYQEKPGQAPRLLMYSASS | (Human) | (Human) | (Human) | (Human) | | PNNYYDGRN | | T | (https://www.nature.co |
| | RATGIPDRFSGSGSATDFTLTINRLEPE | | | | | | VWDY | | | m/articles/s41591-020- |
| | DFAVYYCQQYVEPPFTFGQGTKLEIK | | | | | | | | | 0998-x) |
| CoV2- | QSALTQPRSVSGSPGQSVTISCTGTSS | IGHV3-30 | IGHJ4 | IGLV2-11 | IGLJ2 | 2610 | ARAGGGSYR | 3812 | SAYAGSNN | Seth Zost et al., 2020 |
| 2639 | DVGGYNYVSWYQQHPGKAPKLMIYD | (Human) | (Human) | (Human) | (Human) | | GPFDY | | LV | (https://www.nature.co |
| | VSKRPSGVPDRFSGSKSGNTASLTVSG | | | | | | | | | m/articles/s41591-020- |
| | LQAEDEAEYYCSAYAGSNNLVFGGGT | | | | | | | | | 0998-x) |
| | KLTVL | | | | | | | | | |
| CoV2- | EIVLTQSPGTLSLSPGERATLSCRAGQT | IGHV3-30 | IGHJ3 | IGKV3-20 | IGKJ2 | 2611 | AKSYNGNYYD | 3813 | QQYGSSYT | Seth Zost et al., 2020 |
| 2641 | VSSSYLAWYQHKPGQAPRLLIYGASSR | (Human) | (Human) | (Human) | | | | | | (https://www.nature.co |
| | ATGIPDRFSGSGSGTDFTLTISRLEPED | | | | | | | | | m/articles/s41591-020- |
| | FAVYYCQQYGSSYTFGQGTKLEIK | | | | | | | | | 0998-x) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV2 | SARS- | | SARS-CoV1, | S; non- | B-cells; SARS-CoV2 | 524 | QVQLVESGGGVVQPGRSLRLSCAASGF | 1603 |
| 2643 | | | CoV1 | | SARS-CoV2 | RBD | Human Patient | | TFSSYGMHWVRQAPGKGLEWVAVISY | |
| | | | | | | | | | DGSNKYYADSVKGRFTISRDNAKNSLYL | |
| | | | | | | | | | QMNSLRAEDTAVYYCARGSAGNYYYG | |
| | | | | | | | | | MDVWGQGTTVTVSS | |
| CoV2- | Ab | SARS-CoV2 | SARS- | | SARS-CoV1, | S; non- | B-cells; SARS-CoV2 | 525 | EVQLVQSGAEVKKPGESLKISCKGSGYSF | 1604 |
| 2656 | | | CoV1 | | SARS-CoV2 | RBD | Human Patient | | SDYWIGWVRQMPGKGLEWMGIIYPG | |
| | | | | | | | | | DSDTRYSPSFQGQVTISADKSISTAYLQW | |
| | | | | | | | | | SSLKASDTAMYYCARLTFGGSGSYYFYYN | |
| | | | | | | | | | GMDVWGQGTTVTVSS | |
| CoV2- | Ab | SARS-CoV2 | SARS- | | SARS-CoV1, | S; non- | B-cells; SARS-CoV2 | 526 | EVQLVESGGGLVQPGGSLRLSCAASGFT | 1605 |
| 2660 | | | CoV1 | | SARS-CoV2 | RBD | Human Patient | | FSSYDMHWVRQATGKGLEWVSAIGTA | |
| | | | | | | | | | GDTYYPGSVKGRFTISRENAKNSLYLQM | |
| | | | | | | | | | NSLRAGDTAVYYCARADPYQLLGQHYYY | |
| | | | | | | | | | GMDVWGQGTTVTVSS | |
| CoV2- | Ab | SARS-CoV1, | | | SARS-CoV2 | S; non- | B-cells; SARS-CoV2 | 527 | QVHLVESGGGVVQPGRSLRLSCAASGF | 1606 |
| 2669 | | SARS-CoV2 | | | | RBD | Human Patient | | TFSNYGMHWVRQAPGKGLEWVAVISN | |
| | | | | | | | | | DEFNKFYANSVKGRFTISRDNSKNTVYL | |
| | | | | | | | | | QLNSLRTEDTARYYCAKGGDGSGWAW | |
| | | | | | | | | | DGDNPPTDYWGQGTLVIVSS | |
| CoV2- | Ab | SARS-CoV2 | SARS- | | SARS-CoV1, | S; NTD | B-cells; SARS-CoV2 | 528 | QVTLKESGPVLVKPTETLTLTCAVSGFSLS | 1607 |
| 2673 | | | CoV1 | | SARS-CoV2 | | Human Patient | | NAKMGVSWIRQPPGKALEWLAHIFSN | |
| | | | | | | | | | DEKAYSTSLKTRLTISKDTSKSQVVLTVTN | |
| | | | | | | | | | MDPVDTATYYCARIVLGASGTYPSPGFD | |
| | | | | | | | | | PWGQGTLVTVSS | |
| CoV2- | Ab | SARS-CoV2 | SARS- | | SARS-CoV1, | S; non- | B-cells; SARS-CoV2 | 529 | QVQLVESGGGVVQPGRSLRLSCAASGF | 1608 |
| 2675 | | | CoV1 | | SARS-CoV2 | RBD | Human Patient | | TFSIYGMHWVRQAPGKGLEWVAVISFD | |
| | | | | | | | | | GSNKYYADSVKGRFTISRDNSKNTLYLQ | |
| | | | | | | | | | MNSLRAEDTAVYYCAKDGSGSYYGWFD | |
| | | | | | | | | | PWGQGTLVTVSS | |
| CoV2- | Ab | SARS-CoV2 | SARS- | SARS-CoV2 | | S; non- | B-cells; SARS-CoV2 | 530 | QVQLVQSGAEVKKPGSSVKVSCKASGG | 1609 |
| 2676 | | | CoV1 | (weak) | | RBD | Human Patient | | TFSSYAINWVRQAPGQGLEWMGGIIPIF | |
| | | | | | | | | | GTANYAQKFQGRVTFTADESTSTAYME | |
| | | | | | | | | | VSSLRSEDTAVYYCARSCGDCYSADLDF | |
| | | | | | | | | | WGQGTLVTVSS | |
| CoV2- | Ab | SARS-CoV1, | | SARS-CoV2 | | S; RBD | B-cells; SARS-CoV2 | 531 | QLQLQESGPGLVKPSETLSLTCTVSGGSI | 1610 |
| 2677 | | SARS-CoV2 | | | | | Human Patient | | SSSSYYWGWIRQPPGKGLEWIGSMYYS | |
| | | | | | | | | | GSTYYNPSLKSRVTISVDTSKNQFSLKLSS | |
| | | | | | | | | | VTAADTAVYYCARLLWLRGHFDYWGQ | |
| | | | | | | | | | GTLVTVSS | |
| CoV2- | Ab | SARS-CoV1, | | SARS-CoV2 | | S; RBD | B-cells; SARS-CoV2 | 532 | EVQLVESGGGVVRPGGSLRLSCAASGFI | 1611 |
| 2678 | | SARS-CoV2 | | | | | Human Patient | | FDDYDMTWVRQAPGKGLEWVSGISW | |
| | | | | | | | | | NGGNTGYADSVKGRFTISRDNAKNSLYL | |
| | | | | | | | | | QMNSLRAEDTALYHCAVIMSPIPRYSGY | |
| | | | | | | | | | DWAGGAFDIWGQGTMVTVSS | |
| CoV2- | Ab | SARS-CoV1, | | | SARS-CoV2 | S; non- | B-cells; SARS-CoV2 | 533 | QVQLVESGGGVVQPGRSLRLSCAASGF | 1612 |
| 2681 | | SARS-CoV2 | | | | RBD | Human Patient | | TFSFYAIHWVRQAPGQGLEWAAAISSD | |
| | | | | | | | | | GTYKYYADSVKGRFTISRDNSKNTSYLQ | |
| | | | | | | | | | MNSLRAEDTAVYYCARALNKGFDPWG | |
| | | | | | | | | | QGTLLTVSS | |
| CoV2- | Ab | SARS-CoV2 | SARS- | SARS-CoV2 | | S; RBD | B-cells; SARS-CoV2 | 534 | QMQLVQSGPEVKKPGTSVKVSCKTSGF | 1613 |
| 2684 | | | CoV1 | (weak) | | | Human Patient | | TFTSSAIQWVRQARGQRLEWIGWIVVG | |
| | | | | | | | | | SGNTNYAQKFQERVTITRDMSTSTAYM | |
| | | | | | | | | | ELSSLRSEDTAVYYCAAPHCNRTSCYDAF | |
| | | | | | | | | | DLWGQGTMVTVSS | |
| CoV2- | Ab | SARS-CoV1, | | | SARS-CoV2 | S; non- | B-cells; SARS-CoV2 | 535 | QVQLVQSGAEVKKPGASVQVSCEASGY | 1614 |
| 2685 | | SARS-CoV2 | | | | RBD | Human Patient | | TFTTYYMHWVRQAPGQGLEWMGIINP | |
| | | | | | | | | | SGGSTTYAQKFQGRVTMTRDTSTSTVY | |
| | | | | | | | | | MDLSSLRSEDTAVYYCARDRLGDGSYLG | |
| | | | | | | | | | GGYYGMDVWGQGTTVTVSS | |
| CoV2- | Ab | SARS-CoV2 | SARS- | SARS-CoV2 | | S; RBD | B-cells; SARS-CoV2 | 536 | QVQLVQSGAEVKKPGASVKVSCQASGY | 1615 |
| 2693 | | | CoV1 | (weak) | | | Human Patient | | TFTSYDINWVRQATGQGLEWMGWMK | |
| | | | | | | | | | SNSGNTGYAQKFQGRVTMTRNTSISTA | |
| | | | | | | | | | YMELTSLRSEDTAVYYCARMRSGWPTH | |
| | | | | | | | | | GRPDDLWGRGTLVTVSS | |
| CoV2- | Ab | SARS-CoV2 | SARS- | | SARS-CoV1, | S; RBD | B-cells; SARS-CoV2 | 537 | QVQLVQSGAEVKRPGSSVKVSCKASGG | 1616 |
| 2694 | | | CoV1 | | SARS-CoV2 | | Human Patient | | TFSSYTISWVRQAPGQGLEWMGRIIPIL | |
| | | | | | | | | | AVANYAQKFQGRVTITADKSTSTAYMEL | |
| | | | | | | | | | SSLRSEDTAVYYCARDHSGYYDSTSLMSP | |
| | | | | | | | | | FFDYWGQGTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | DIQMTQSPSSLSASVGDRVTITCRASQ | IGHV3-30 | IGHJ6 | IGKV1-39 | IGKJ5 | 2612 | ARGSAGNYYY | 3814 | QQSYSTPG | Seth Zost et al., 2020 |
| 2643 | SISSYLNWYQQKPGKAPKLLIYAASSLQ | (Human) | (Human) | (Human) | (Human) | | GMDV | | T | (https://www.nature.co |
| | SGVPSRFSGSGSGTDFTLTISSLQPEDY | | | | | | | | | m/articles/s41591-020- |
| | ATYYCQQSYSTPGTFGQGTRLEIK | | | | | | | | | 0998-x) |
| CoV2- | EIVLTQSPGTLSLSPGERATLSCRASQS | IGHV5-51 | IGHJ6 | IGKV3-20 | IGKJ1 | 2613 | ARLTFGGSGS | 3815 | QQYGRSS | Seth Zost et al., 2020 |
| 2656 | VSSSYLAWYQQKPGQAPRLLIYGASSR | (Human) | (Human) | (Human) | (Human) | | YYFYYNGMD | | GT | (https://www.nature.co |
| | ATGIPDRFSGSGSGTDFTLTISRLEPED | | | | | | V | | | m/articles/s41591-020- |
| | FAVYYCQQYGRSSGTFGQGTKVEIK | | | | | | | | | 0998-x) |
| CoV2- | EIVLTQSPGTLSLSPGERATLSCRASQS | IGHV3-13 | IGHJ6 | IGKV3-20 | IGKJ5 | 2614 | ARADPYQLLG | 3816 | QQYGSSPL | Seth Zost et al., 2020 |
| 2660 | VSSSYLAWYQQKPGQAPRLLIYGASSR | (Human) | (Human) | (Human) | (Human) | | QHYYYGMDV | | IT | (https://www.nature.co |
| | ATGIPDRFSGSGSGTDFTLTISRLEPED | | | | | | | | | m/articles/s41591-020- |
| | FAVYYCQQYGSSPLITFGQGTRLEIK | | | | | | | | | 0998-x) |
| CoV2- | DIVMTQSPDFLAVSLGERATISCKSSQS | IGHV3-30 | IGHJ4 | IGKV4-1 | IGKJ4 | 2615 | AKGGDGSG | 3817 | QQYYTAPL | Seth Zost et al., 2020 |
| 2669 | VLYTPKNKNYLAWYKQKPGQPPKVLIY | (Human) | (Human) | (Human) | (Human) | | WAWDGDNP | | T | (https://www.nature.co |
| | WASTRESGVPDRFSGSGSGTDFTLIISS | | | | | | PTDY | | | m/articles/s41591-020- |
| | LQAEDAAVYYCQQYYTAPLTFGGGTR | | | | | | | | | 0998-x) |
| | VEIK | | | | | | | | | |
| CoV2- | QTVVTQEPSLTVSPGGTVTLTCASSAG | IGHV2-26 | IGHJ5 | IGLV7-43 | IGLJ3 | 2616 | ARIVLGASGT | 3818 | LLYYGGA | Seth Zost et al., 2020 |
| 2673 | AVTSGYYPNWFQQKPGQAPRALIYST | (Human) | (Human) | (Human) | (Human) | | YPSPGFDP | | WV | (https://www.nature.co |
| | ANKHSWTPARFSGSLLGGKAALTLSG | | | | | | | | | m/articles/s41591-020- |
| | VQPEDEAEYYCLLYYGGAWVFGGGTK | | | | | | | | | 0998-x) |
| | LTVL | | | | | | | | | |
| CoV2- | EIVLTQSPDFQSVTPKEKVTITCRASQN | IGHV3-30 | IGHJ5 | IGKV6-21 | IGKJ4 | 2617 | AKDGSGSYYG | 3819 | HQSSSLPP | Seth Zost et al., 2020 |
| 2675 | IGSSLHWYQQKPDQSPKVIIKYASQSF | (Human) | (Human) | (Human) | (Human) | | WFDP | | T | (https://www.nature.co |
| | SGVPSRFSGSGSGTDFTLTINSLEAEDA | | | | | | | | | m/articles/s41591-020- |
| | ATYYCHQSSSLPPTFGGGTKVEIK | | | | | | | | | 0998-x) |
| CoV2- | EIVLTQSPATLSLSPGERATLSCRASQSI | IGHV1-69 | IGHJ4 | IGKV3-11 | IGKJ3 | 2618 | ARSCGDCYSA | 3820 | QRRSNWP | Seth Zost et al., 2020 |
| 2676 | SSFLAWYQQKPGQAPRLLIYDASNRA | (Human) | (Human) | (Human) | (Human) | | DLDE | | PFT | (https://www.nature.co |
| | TGIPARFSGSGSGTDFTLTISSLEPEDFA | | | | | | | | | m/articles/s41591-020- |
| | VYYCQRRSNWPPFTFGPGTKVDIK | | | | | | | | | 0998-x) |
| CoV2- | NFMLTQPHSVSESPGKTVTISCTGSSG | IGHV4-39 | IGHJ4 | IGLV6-57 | IGLJ3 | 2619 | ARLLWLRGHF | 3821 | QSYDSSNY | Seth Zost et al., 2020 |
| 2677 | SIASNYVQWYQQRPGSAPTTVIYEDN | (Human) | (Human) | (Human) | (Human) | | DY | | WV | (https://www.nature.co |
| | QRPSGVPDRFSGSIDSSSNSASLTISGL | | | | | | | | | m/articles/s41591-020- |
| | KTEDEADYYCQSYDSSNYWVFGGGTK | | | | | | | | | 0998-x) |
| | LTVL | | | | | | | | | |
| CoV2- | SSELTQDPAVSVALGQTVRITCQGDSL | IGHV3-20 | IGHJ3 | IGLV3-19 | IGLJ3 | 2620 | AVIMSPIPRY | 3822 | NSRDSSGN | Seth Zost et al., 2020 |
| 2678 | RSYYASWYQQKPGQVPILVIYDKNNR | (Human) | (Human) | (Human) | (Human) | | SGYDWAGGAF | | AVV | (https://www.nature.co |
| | PSGIPDRFSGSSSGNTASLTITGAQAED | | | | | | DI | | | m/articles/s41591-020- |
| | EADYYCNSRDSSGNAVVFGGGTKLTV | | | | | | | | | 0998-x) |
| | L | | | | | | | | | |
| CoV2- | DIVMTQSPDSLAVSLGERATINCKSSQ | IGHV3-30 | IGHJ5 | IGKV4-1 | IGKJ2 | 2621 | ARALNKGFDP | 3823 | QQYYSSPY | Seth Zost et al., 2020 |
| 2681 | SVLYSSNNKNYLAWYQQKPGQPPKLLI | (Human) | (Human) | (Human) | (Human) | | | | T | (https://www.nature.co |
| | YWASTRESGVPDRFSGSGSGTDFTLTI | | | | | | | | | m/articles/s41591-020- |
| | SSLQAEDVAVYYCQQYYSSPYTFGQGT | | | | | | | | | 0998-x) |
| | KLEIK | | | | | | | | | |
| CoV2- | EIVLTQSPGTLSLSPGERATLSCRASQS | IGHV1-58 | IGHJ3 | IGKV3-20 | IGKJ1 | 2622 | AAPHCNRTSC | 3824 | QQYNNW | Seth Zost et al., 2020 |
| 2684 | VSSSYLGWYQQKPGQAPRLLIYGASSR | (Human) | (Human) | (Human) | (Human) | | YDAFDL | | WRT | (https://www.nature.co |
| | ATGIPDRFSGSGSGTDFTLTISRLESED | | | | | | | | | m/articles/s41591-020- |
| | FALYYCQQYNNWWRTFGQGTKVEIK | | | | | | | | | 0998-x) |
| CoV2- | EIVLTQSPGTLSLSPGERATLSCRASQS | IGHV1-46 | IGHJ6 | IGKV3-20 | IGKJ4 | 2623 | ARDRLGDGSY | 3825 | QQYGSSPR | Seth Zost et al., 2020 |
| 2685 | VSSSYLAWYQQKRGQAPRLLIYGASSR | (Human) | (Human) | (Human) | (Human) | | LGGGYYGMD | | LT | (https://www.nature.co |
| | ATGIPDRFSGSGSGTDFTLTISRLEPED | | | | | | V | | | m/articles/s41591-020- |
| | FAVYYCQQYGSSPRLTFGGGTKVEIK | | | | | | | | | 0998-x) |
| CoV2- | QSVLTQPPSASGTPGQRVTISCSGSNS | IGHV1-8 | IGHJ5 | IGLV1-44 | IGLJ2 | 2624 | ARMRSGWPT | 3826 | AVWDDSL | Seth Zost et al., 2020 |
| 2693 | NIGSYTVNWYQQLPGTAPKLLIYDNN | (Human) | (Human) | (Human) | (Human) | | HGRPDDL | | NGLV | (https://www.nature.co |
| | QRTSGVPDRLSGSKSGTSASLAISGLQS | | | | | | | | | m/articles/s41591-020- |
| | EDEANYYCAVWDDSLNGLVFGGGTK | | | | | | | | | 0998-x) |
| | LTVL | | | | | | | | | |
| CoV2- | QSVLTQPPSVSGAPGQRVTISCTGSSS | IGHV1-69 | IGHJ4 | IGLV1-40 | IGLJ3 | 2625 | ARDHSGYYDS | 3827 | QSYDSSLN | Seth Zost et al., 2020 |
| 2694 | NIGAGYDVHWYQQLPGTAPKLLIFGN | (Human) | (Human) | (Human) | (Human) | | TSLMSPFFDY | | GDV | (https://www.nature.co |
| | TNRPSGVPDRFSGSKSGTSTSLAITGLQ | | | | | | | | | m/articles/s41591-020- |
| | AEDEADYYCQSYDSSLNGDVFGGGTK | | | | | | | | | 0998-x) |
| | LTVL | | | | | | | | | |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV2 | SARS- | | SARS-CoV1, | S; non- | B-cells; SARS-CoV2 | 538 | QVQLVQSGAEVKKPGASVKVSCKASGY | 1617 |
| 2697 | | | CoV1 | | SARS-CoV2 | RBD | Human Patient | | TFTSHYMHWVRQAPGQGLEWMGIINP | |
| | | | | | | | | | SGGSTSYAQKFQGRVTMTGDTSTSTVY | |
| | | | | | | | | | MELSSLRSEDTAVYYCARDLAGVPAALG | |
| | | | | | | | | | CWFDPWGQGTLVTVSS | |
| CoV2- | Ab | SARS-CoV2 | SARS- | | SARS-CoV1, | S; non- | B-cells; SARS-CoV2 | 539 | QVQLVESGGGVVQPGRSLRLSCAASGF | 1618 |
| 2700 | | | CoV1 | | SARS-CoV2 | RBD | Human Patient | | TFSTYAMHWVRQAPGKGLEWVAVISY | |
| | | | | | | | | | DGGNKYYADSVKGRFTISRDNSKNTLYL | |
| | | | | | | | | | QMNSLRAEDTAVYYCAKNLGPYCSGGT | |
| | | | | | | | | | CYSLVGDYWGQGTLVTVSS | |
| CoV2- | Ab | SARS-CoV2 | SARS- | | SARS-CoV1, | S; RBD | B-cells; SARS-CoV2 | 540 | EVQLVESGGGLVQPGGSLRLSCAASGFT | 1619 |
| 2703 | | | CoV1 | | SARS-CoV2 | | Human Patient | | FSSYDMHWVRQATGKGLEWVSAIGTA | |
| | | | | | | | | | GDTYYPGSVKGRFTISRENAKNSLYLQM | |
| | | | | | | | | | NSLRAGDTAVYYCARARGGYNWNFDY | |
| | | | | | | | | | WGQGTLVTVSS | |
| CoV2- | Ab | SARS-CoV1, | | | SARS-CoV2 | S; RBD | B-cells; SARS-CoV2 | 541 | QVQLVESGGGVVQPGRSLRLSCAASGF | 1620 |
| 2705 | | SARS-CoV2 | | | | | Human Patient | | TFSSYAMHWVRQAPGKGLEWVAVISY | |
| | | | | | | | | | DGSYKYFADSVKGRFTISRDNSKNTLYLQ | |
| | | | | | | | | | MNSLRAEDTAVYYCARDQGTVVTHFDY | |
| | | | | | | | | | WGQGTLVTVSS | |
| CoV2- | Ab | SARS-CoV1, | | | SARS-CoV2 | S; RBD | B-cells; SARS-CoV2 | 542 | QVQLVESGGGVVQPGRSLRLSCAASGF | 1621 |
| 2709 | | SARS-CoV2 | | | | | Human Patient | | TFSNYGMHWVRQAPGKGLEWVAVMS | |
| | | | | | | | | | YDGSNKYYADSVKGRFTISRDNSKNTLYL | |
| | | | | | | | | | QMNSLRAEDTAVYYCAKNLGPYCSGGT | |
| | | | | | | | | | CYSLVGDYWGQGTLVTVSS | |
| CoV2- | Ab | SARS-CoV2 | SARS- | | SARS-CoV1, | S; RBD | B-cells; SARS-CoV2 | 543 | QVQLVQSGAEVKKPGASVKVSCKASGY | 1622 |
| 2710 | | | CoV1 | | SARS-CoV2 | | Human Patient | | TFTSYDINWVRQATGQGLEWMGWMS | |
| | | | | | | | | | PNSGNTGYAQKFQGRVTMTRDTSISTA | |
| | | | | | | | | | YMELNSLRSEDTAVYYCARMRSGWPTH | |
| | | | | | | | | | GRPDDHWGQGTLVTVSS | |
| CoV2- | Ab | SARS-CoV1, | | | SARS-CoV2 | S; RBD | B-cells; SARS-CoV2 | 544 | EVQLVESGGGVVQPGGSLRLSCAASGF | 1623 |
| 2713 | | SARS-CoV2 | | | | | Human Patient | | TEDDYAMHWVRQAPGKGLEWVSLISG | |
| | | | | | | | | | DGGNTYYADSVKGRFTISRDNSKNSLYL | |
| | | | | | | | | | QMNSLRTEDTALYYCAKDEMAYPPSHH | |
| | | | | | | | | | YYYYYMDVWGKGTTVTVSS | |
| CoV2- | Ab | SARS-CoV1, | | | SARS-CoV2 | S; RBD | B-cells; SARS-CoV2 | 545 | QVQLVQSGAEVKTPGASVKVSCKASGY | 1624 |
| 2717 | | SARS-CoV2 | | | | | Human Patient | | TFTSYDINWVRQATGQGPEWMGWM | |
| | | | | | | | | | NPNSGNTGYAHKFQGRVTMTRNTSIST | |
| | | | | | | | | | AYMELSSLRSEDTAVYYCARGPSILTGFY | |
| | | | | | | | | | NPLDYWGQGTLVTVSS | |
| CoV2- | Ab | SARS-CoV2 | SARS- | | SARS-CoV1, | S; non- | B-cells; SARS-CoV2 | 546 | QLQLQESGPGLVKPSETLSLTCTVSGGSI | 1625 |
| 2718 | | | CoV1 | | SARS-CoV2 | RBD | Human Patient | | SSSNYYWGWIRQPPGKGLEWIGTIHYS | |
| | | | | | | | | | GISYYNPSLKSRVTISVDTSNNKFSLELSS | |
| | | | | | | | | | VTAADTAVYFCARRTYYDLWSAYSSTAY | |
| | | | | | | | | | YCMDVWGKGTTVTVSS | |
| CoV2- | Ab | SARS-CoV1, | | SARS-CoV2 | | S; RBD | B-cells; SARS-CoV2 | 547 | QVRLVQSGAEVKKPGSSVKVSCKASGG | 1626 |
| 2722 | | SARS-CoV2 | | | | | Human Patient | | TFSDYAISWVRQAPGQGLEWMGGIIPIF | |
| | | | | | | | | | GTANYAQKFQGRVTITADEFTITAYMEL | |
| | | | | | | | | | SSLRSEDTAVYYCARLSGSGWLGYAMD | |
| | | | | | | | | | VWGQGTTVTVSS | |
| CoV2- | Ab | SARS-CoV2 | SARS- | | SARS-CoV1, | S; non- | B-cells; SARS-CoV2 | 548 | QVQLVESGGGVVQPGRSLRLSCAASGF | 1627 |
| 2726 | | | CoV1 | | SARS-CoV2 | RBD | Human Patient | | TFSSYAMHWVRQAPGKGLEWVAVISN | |
| | | | | | | | | | DGRNKYYADSVKGRLTISRDNSKNTLYL | |
| | | | | | | | | | QMNSLRAEDTAVYYCARPSNWYFDLW | |
| | | | | | | | | | GRGTLVTVSS | |
| CoV2- | Ab | SARS-CoV2 | SARS- | SARS-CoV2 | | S; RBD | B-cells; SARS-CoV2 | 549 | EVQLVESGGGLVQPGRSLTLSCAASGFT | 1628 |
| 2730 | | | CoV1 | (weak) | | | Human Patient | | FDDYTMHWVRQAPGKGLEWVSGIDW | |
| | | | | | | | | | NGGTIGYADSVKGRFTISRDNAKNSLYL | |
| | | | | | | | | | QMNSLRAEDTALYYCAKAGYYAYVWGS | |
| | | | | | | | | | YRFEYFDNWGQGTLVTVSS | |
| CoV2- | Ab | SARS-CoV2 | SARS- | SARS-CoV2 | | S; RBD | B-cells; SARS-CoV2 | 550 | EVQLVESGGGLVQPGGSLRLSCAASGVT | 1629 |
| 2733 | | | CoV1 | | | | Human Patient | | VSSNYMSWVRQAPGKGLEWVSLIYSGG | |
| | | | | | | | | | STFYADSVKGRFTISRHNSKNTLYLQMN | |
| | | | | | | | | | SLRPEDTAVYYCARGPEPDAFDIWGQG | |
| | | | | | | | | | TMVTVSS | |
| CoV2- | Ab | SARS-CoV1, | | SARS-CoV2 | | S; RBD | B-cells; SARS-CoV2 | 551 | QVQLQESGPGLVKPSETLSLTCTVSGGS | 1630 |
| 2734 | | SARS-CoV2 | | (weak) | | | Human Patient | | VSSGSYYWSWIRQPPGKGLECIGYIYYSG | |
| | | | | | | | | | SSNYNPSLKSRVTISVDTSKNQFSLKMSS | |
| | | | | | | | | | VTAADTAVYYCAGSPVPPTIVGASYWG | |
| | | | | | | | | | QGTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | EIVLTQSPATLSLSPGERATLSCRASQS | IGHV1-46 | IGHJ5 | IGKV3-11 | IGKJ3 | 2626 | ARDLAGVPA | 3828 | QQRSNWP | Seth Zost et al., 2020 |
| 2697 | VSSYLAWYQQKPGQAPRLLIYDAYKR | (Human) | (Human) | (Human) | (Human) | | ALGCWFDP | | LIFT | (https://www.nature.co |
| | DTGIPARFSGSGSGTDFTLTISSLEPEDF | | | | | | | | | m/articles/s41591-020- |
| | AVYYCQQRSNWPLIFTFGPGTKVDIK | | | | | | | | | 0998-x) |
| CoV2- | QSVLTQPPSVSGVPGQRVTVSCTGSSS | IGHV3-30- | IGHJ4 | IGLV1-40 | IGLJ1 | 2627 | AKNLGPYCSG | 3829 | QSYDSSLS | Seth Zost et al., 2020 |
| 2700 | NIGAGFDVYWYQQFLGTAPKLLIYGN | 3 | (Human) | (Human) | (Human) | | GTCYSLVGDY | | GYV | (https://www.nature.co |
| | NNRPSGVPDRFSASKSGTSASLAITGL | (Human) | | | | | | | | m/articles/s41591-020- |
| | QAEDEADYYCQSYDSSLSGYVFGTGTK | | | | | | | | | 0998-x) |
| | VTVL | | | | | | | | | |
| CoV2- | DIQMTQSPSSLSASVGDRVTITCRASQ | IGHV3-13 | IGHJ4 | IGKV1-39 | IGKJ5 | 2628 | ARARGGYNW | 3830 | QQSYSTPPI | Seth Zost et al., 2020 |
| 2703 | SISSYLNWYQQKPGKAPKLLIYDASSLQ | (Human) | (Human) | (Human) | (Human) | | NFDY | | T | (https://www.nature.co |
| | SGVPSRFSGSGSGTDFTLTISSLQPEDF | | | | | | | | | m/articles/s41591-020- |
| | ATYYCQQSYSTPPITFGQGTRLEIK | | | | | | | | | 0998-x) |
| CoV2- | DIQMTQSPSSLSTSVGDRVTITCRASQ | IGHV3-30 | IGHJ4 | IGKV1-39 | IGKJ1 | 2629 | ARDQGTVVT | 3831 | QQSYSTPP | Seth Zost et al., 2020 |
| 2705 | SIRSYLNWYQQKPGKAPKLLIYVASSL | (Human) | (Human) | (Human) | (Human) | | HFDY | | WT | (https://www.nature.co |
| | QSGVPSRFSGSGSGTDFTLTISSLQPED | | | | | | | | | m/articles/s41591-020- |
| | FATYYCQQSYSTPPWTFGQGTKVEIK | | | | | | | | | 0998-x) |
| CoV2- | DIQMTQSPSSLSASVGDRVTITCQASQ | IGHV3-30 | IGHJ4 | IGKV1-33 | IGKJ3 | 2630 | AKNLGPYCSG | 3832 | QQYANLPF | Seth Zost et al., 2020 |
| 2709 | DISNYLNWYQQKPGKAPKLLIYDASNL | (Human) | (Human) | (Human) | (Human) | | GTCYSLVGDY | | T | (https://www.nature.co |
| | ETGVPSRFSGSGSGTDFTFTISSLQPED | | | | | | | | | m/articles/s41591-020- |
| | IATYYCQQYANLPFTFGPGTKVDIK | | | | | | | | | 0998-x) |
| CoV2- | QSVLTQPPSASGTPGQRVTISCSGSNS | IGHV1-8 | IGHJ4 | IGLV1-44 | IGLJ2 | 2631 | ARMRSGWPT | 3833 | VAWDDSR | Seth Zost et al., 2020 |
| 2710 | NIGSYTVNWYQQLPGTAPKLLIFGNN | (Human) | (Human) | (Human) | (Human) | | HGRPDDH | | NGLV | (https://www.nature.co |
| | QRPSGVPDRFSGSKSGTSASLAISGLQ | | | | | | | | | m/articles/s41591-020- |
| | SEDEADYYCVAWDDSRNGLVFGGGA | | | | | | | | | 0998-x) |
| | KLTVL | | | | | | | | | |
| CoV2- | SYELTQPPSVSVSPGQTARITCSTDALP | IGHV3-43 | IGHJ6 | IGLV3-25 | IGLJ2 | 2632 | AKDEMAYPP | 3834 | QSVDSSGT | Seth Zost et al., 2020 |
| 2713 | NEYIYWYQQKPGQAPVLVIYKDSERPS | (Human) | (Human) | (Human) | (Human) | | SHHYYYYYMD | | YPHVI | (https://www.nature.co |
| | GIPERFSGSSSGTTVTLTISGVQAEDEA | | | | | | V | | | m/articles/s41591-020- |
| | DYYCQSVDSSGTYPHVIFGGGTKLTVL | | | | | | | | | 0998-x) |
| CoV2- | DIQMTQSPSSLSASVGDRVTITCRASQ | IGHV1-8 | IGHJ4 | IGKV1-39 | IGKJ2 | 2633 | ARGPSILTGFY | 3835 | QQSYSTPY | Seth Zost et al., 2020 |
| 2717 | SIISYLNWYHQKPGKAPKLLIYAASSLQ | (Human) | (Human) | (Human) | (Human) | | NPLDY | | T | (https://www.nature.co |
| | SGVPSRFSGSGSGTDFTLTISSLQPEDF | | | | | | | | | m/articles/s41591-020- |
| | ATYYCQQSYSTPYTFGQGTKLEIK | | | | | | | | | 0998-x) |
| CoV2- | DIQLTQSPSFLSASVGDRVTITCRASQG | IGHV4-39 | IGHJ6 | IGKV1-9 | IGKJ4 | 2634 | ARRTYYDLWS | 3836 | QQLNSYPL | Seth Zost et al., 2020 |
| 2718 | ISSYLAWYQQKPGKAPKLLIYAASTLQS | (Human) | (Human) | (Human) | (Human) | | AYSSTAYYCM | | T | (https://www.nature.co |
| | GVPSRFSGSGSGTEFTLTISSLQPEDFA | | | | | | DV | | | m/articles/s41591-020- |
| | TYYCQQLNSYPLTFGGGTKVEIK | | | | | | | | | 0998-x) |
| CoV2- | QSALTQPASVSGSPGQSITISCTGTSSD | IGHV1-69 | IGHJ6 | IGLV2-14 | IGLJ2 | 2635 | ARLSGSGWL | 3837 | TSYTSSSTL | Seth Zost et al., 2020 |
| 2722 | VGGYNFVSWYQQHPGKAPKLIIYDVS | (Human) | (Human) | (Human) | (Human) | | GYAMDV | | NVV | (https://www.nature.co |
| | NRPSGVSNRFSGSKSGNTASLTISGLQ | | | | | | | | | m/articles/s41591-020- |
| | AEDEADYYCTSYTSSSTLNVVFGGGTK | | | | | | | | | 0998-x) |
| | LTVL | | | | | | | | | |
| CoV2- | QSALTQPASVSGSPGQSITISCTGTSSD | IGHV3-30 | IGHJ2 | IGLV2-23 | IGLJ2 | 2636 | ARPSNWYFD | 3838 | CSYASSSIV | Seth Zost et al., 2020 |
| 2726 | VGSYNLVSWYQQHPGKAPKLMIYEVS | (Human) | (Human) | (Human) | (Human) | | L | | V | (https://www.nature.co |
| | KRPSGVSNRFSGSKSGNTASLTISGLQ | | | | | | | | | m/articles/s41591-020- |
| | AEDEADYYCCSYASSSIVVFGGGTKLTV | | | | | | | | | 0998-x) |
| | L | | | | | | | | | |
| CoV2- | DIQMTQSPSSLSASVGDRVTITCRASQ | IGHV3-9 | IGHJ4 | IGKV1-39 | IGKJ1 | 2637 | AKAGYYAYV | 3839 | QQSYSTPP | Seth Zost et al., 2020 |
| 2730 | SISSYLNWYQQKPGKAPKLLLYAASSL | (Human) | (Human) | (Human) | (Human) | | WGSYRFEYFD | | WT | (https://www.nature.co |
| | QSGVPSRFSGSGSGTDFTLTISSLQPED | | | | | | N | | | m/articles/s41591-020- |
| | FATYYCQQSYSTPPWTFGQGTKVEIK | | | | | | | | | 0998-x) |
| CoV2- | DIQLTQSPSFLSASVGDRVTITCRASQG | IGHV3-53 | IGHJ3 | IGKV1-9 | IGKJ3 | 2638 | ARGPEPDAFD | 3840 | QQLNSYSF | Seth Zost et al., 2020 |
| 2733 | ISSYLAWYQQKPGKAPKLLIYAASTLQS | (Human) | (Human) | (Human) | (Human) | | | | ET | (https://www.nature.co |
| | GVPSRFSGSGSGTEFTLTISSLQPEDFA | | | | | | | | | m/articles/s41591-020- |
| | TYYCQQLNSYSFETFGPGTKVAIK | | | | | | | | | 0998-x) |
| CoV2- | NFMLTQPHSVSESPGKTVTFSCTGSSG | IGHV4-61 | IGHJ4 | IGLV6-57 | IGLJ2 | 2639 | AGSPVPPTIV | 3841 | QSYDGINR | Seth Zost et al., 2020 |
| 2734 | SIASNYVQWYQQRPGSAPTTVIYEDN | (Human) | (Human) | (Human) | (Human) | | GASY | | WLV | (https://www.nature.co |
| | QRPSGVPDRFSGSIDSSSNSASLTISGL | | | | | | | | | m/articles/s41591-020- |
| | KTEDEADYYCQSYDGINRWLVFGGGT | | | | | | | | | 0998-x) |
| | KLTVL | | | | | | | | | |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2 | Ab | SARS-CoV1, | | | SARS-CoV2 | S; RBD | B-cells; SARS-CoV2 | 552 | QVQLVESGGGVVQPGRSLRLSCAASGF | 1631 |
| 2736 | | SARS-CoV2 | | | | | Human Patient | | TFSDYAMHWVRQAPGKGLEWVADISF | |
| | | | | | | | | | DGSNKYYADSVKGRFTISRDSSENTLYLQ | |
| | | | | | | | | | MDSLRADDTAVYYCARDLSTTWYLEM | |
| | | | | | | | | | WGPDAFDIWGQGTVVTVSS | |
| CoV2- | Ab | SARS-CoV1, | | | SARS-CoV2 | S; RBD | B-cells; SARS-CoV2 | 553 | QVQLVESGGGVVQPGRSLRLSCAASGF | 1632 |
| 2740 | | SARS-CoV2 | | | | | Human Patient | | TFRRYGMYWVRQAPGKGLEWVAVISY | |
| | | | | | | | | | DGTDKYYTDSVKGRFTISRDNSKNTLYLQ | |
| | | | | | | | | | MNSLRAEDTAVYYCAKKGGPYCGGGN | |
| | | | | | | | | | CYAGYFDYWGQGTLVTVSS | |
| CoV2- | Ab | SARS-CoV1, | | | SARS-CoV2 | S; RBD | B-cells; SARS-CoV2 | 554 | QVQLQQWGAGLLKPSETLSLTCAVSGG | 1633 |
| 2749 | | SARS-CoV2 | | | | | Human Patient | | SFSAYYWSWIRQPPGKGLEWIGEINHS | |
| | | | | | | | | | GSTNYNPSLRSRVTISVDTSKNQFSLKLSS | |
| | | | | | | | | | VTAADTAVYYCARVGYSQGYYYYYMDV | |
| | | | | | | | | | WGKGTTVTVSS | |
| CoV2- | Ab | SARS-CoV2 | SARS- | | SARS-CoV1, | S; RBD | B-cells; SARS-CoV2 | 555 | EVQVVESGGGLVQPGRSLRLSCVSSGFI | 1634 |
| 2751 | | | CoV1 | | SARS-CoV2 | | Human Patient | | FDDYVMHWVRQRPGKGLEWVAGITYN | |
| | | | | | | | | | GGILGYGDSVKGRFIIARDNVRGFLSLQ | |
| | | | | | | | | | MGDLRTEDTALYYCARDYCSSTTCPAET | |
| | | | | | | | | | YYYMDVWGKGTAVTVSS | |
| CoV2- | Ab | SARS-CoV2 | SARS- | SARS-CoV2 | | S; RBD | B-cells; SARS-CoV2 | 556 | EVQLVESGGGLIQPGGSLRLSCAASEVT | 1635 |
| 2752 | | | CoV1 | | | | Human Patient | | VSSNYMSWVRQAPGKGLEWVSLIYSGG | |
| | | | | | | | | | TTYYADSVKGRFTISRDNSKNTLYLQMN | |
| | | | | | | | | | SLRAEDTAVYYCARDFLRWHDLWGQG | |
| | | | | | | | | | TLVTVSS | |
| CoV2- | Ab | SARS-CoV1, | | | SARS-CoV2 | S; RBD | B-cells; SARS-CoV2 | 557 | EVQLVESGGGLVQPGGSLRLSCAASGFT | 1636 |
| 2753 | | SARS-CoV2 | | | | | Human Patient | | FSSYDMHWVRQATGKGLEWVSTIGTA | |
| | | | | | | | | | GDTYYPDSVKGRFTISRENAKNSLFLQM | |
| | | | | | | | | | NSLRAGDTAVYYCARVDFDILTGYYSNW | |
| | | | | | | | | | GQGTLVTVSS | |
| CoV2- | Ab | SARS-CoV2 | SARS- | | SARS-CoV1, | S; RBD | B-cells; SARS-CoV2 | 558 | EAQLLESGGALVQPGGSLRLSCAASGFT | 1637 |
| 2756 | | | CoV1 | | SARS-CoV2 | | Human Patient | | FSCCAMGWVRQAPGRGLEWVSSIHDD | |
| | | | | | | | | | GVGTFYAVSVKGRFSISRDNSKNTVYLQ | |
| | | | | | | | | | MNGLRAEDTGVYYCAKWAGPIVMKYY | |
| | | | | | | | | | LQYWGQGALVTVSS | |
| CoV2 | Ab | SARS-CoV1, | | | SARS-CoV2 | S; RBD | B-cells; SARS-CoV2 | 559 | EVQLVESGGGLVQPGGSLRLSCAASGFT | 1638 |
| 2758 | | SARS-CoV2 | | | | | Human Patient | | FSSYDMHWVRQATGKGLEWVSAIGTA | |
| | | | | | | | | | GDTYYPDSVKGRFTISRENAKNSLYLQM | |
| | | | | | | | | | NSLRAGDTAVYYCARGGDSGYDLGAWY | |
| | | | | | | | | | FDLWGRGTLVTVSS | |
| CoV2- | Ab | SARS-CoV2 | SARS- | | SARS-CoV1, | S; RBD | B-cells; SARS-CoV2 | 560 | EVQLVQSGAEVKKPGESLKISCKGSGYSF | 1639 |
| 2759 | | | CoV1 | | SARS-CoV2 | | Human Patient | | TNYWIGWVRQMPGKGLEWMGIIYPG | |
| | | | | | | | | | DSDTRYSPSFQGQVTISADKSISTAYLQW | |
| | | | | | | | | | SSLKASDTAMYYCARTPTLYNWFHPWG | |
| | | | | | | | | | QGTPVTVSS | |
| CoV2- | Ab | SARS-CoV2 | SARS- | SARS-CoV2 | | S; RBD | B-cells; SARS-CoV2 | 561 | QVTLRESGPALVKPTQTLTLTCTFSGFSLS | 1640 |
| 2760 | | | CoV1 | (weak) | | | Human Patient | | TSGLCVSWIRQPPGKALEWLARIDWDD | |
| | | | | | | | | | DKYYNTSLRTRLTISKDTSKNQVVLTMTN | |
| | | | | | | | | | MDPVDTATYYCARATTFFYGMDVWGQ | |
| | | | | | | | | | GTTVTVSS | |
| CoV2- | Ab | SARS-CoV2 | SARS- | | SARS-CoV1, | S; RBD | B-cells; SARS-CoV2 | 562 | QLQLQESGPGLVKPSETLSLTCTVSGGSI | 1641 |
| 2762 | | | CoV1 | | SARS-CoV2 | | Human Patient | | SSSSYYWGWIRQPPGKGLEWIGSIYYSG | |
| | | | | | | | | | ITYYNPSLKSRVTISVDTSKNQFSLKLSSV | |
| | | | | | | | | | TAADTAVYYCARHQRYCSSSSCHVWDYW | |
| | | | | | | | | | GQGTLVTVSS | |
| CoV2- | Ab | SARS-CoV1, | | | SARS-CoV2 | S; RBD | B-cells; SARS-CoV2 | 563 | EVQLVESGGGLVQPGRSLRLSCAASGFT | 1642 |
| 2765 | | SARS-CoV2 | | | | | Human Patient | | FDDYAMNWVRQPPGKGLEWVSGISW | |
| | | | | | | | | | NSDSIGYADSVKGRFTISRDNAKNSLYLQ | |
| | | | | | | | | | MNSLRAEDTAMYYCAKGRGAGYTSYM | |
| | | | | | | | | | DVWGKGTTVTVSS | |
| CoV2- | Ab | SARS-CoV2 | SARS- | | SARS-CoV1, | S; RBD | B-cells; SARS-CoV2 | 564 | EVQLVESGGGLVQPGGSLRLSCAASGFT | 1643 |
| 2767 | | | CoV1 | | SARS-CoV2 | | Human Patient | | VSSNYMSWVRQAPGKGLEWVSVIYPG | |
| | | | | | | | | | GSAFYADSVKGRFTISRHNSNNTLCLQM | |
| | | | | | | | | | NSLRTEDTAVYYCARSYDILTGYRDAFDI | |
| | | | | | | | | | WGQGTMVTVSS | |
| CoV2- | Ab | SARS-CoV2 | SARS- | | SARS-CoV1, | S; RBD | B-cells; SARS-CoV2 | 565 | QVQLVESGGGVVQPGRSLRLSCAASGF | 1644 |
| 2768 | | | CoV1 | | SARS-CoV2 | | Human Patient | | TFSSYAMHWVRQAPGKGLEWVAVISY | |
| | | | | | | | | | DGSNKYYADSVKGRFTISRDNSKNTLYL | |
| | | | | | | | | | QMNSLRAEDTAVYYCARDDNSPQGSG | |
| | | | | | | | | | WYFYYYYAMDVWGQGTTVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2 | DIQMTQSPSTLSASIGDRVTITCRASQS | IGHV3-30- | IGHJ3 | IGKV1-5 | IGKJ2 | 2640 | ARDLSTTWYL | 3842 | QQYNSYPY | Seth Zost et al., 2020 |
| 2736 | ISSWLAWYQQIPGKAPKLLIYKASSLES | 3 | (Human) | (Human) | (Human) | | EMWGPDAF | | T | (https://www.nature.co |
| | GVPSRFSGSGSGTEFTLTISSLQPDDFA | (Human) | | | | | DI | | | m/articles/s41591-020- |
| | TYYCQQYNSYPYTFGQGTKLEIK | | | | | | | | | 0998-x) |
| CoV2- | DIQMTQSPSSLSASIGDRVTITCRASQ | IGHV3-30 | IGHJ4 | IGKV1-39 | IGKJ2 | 2641 | AKKGGPYCG | 3843 | QQSSSSPIT | Seth Zost et al., 2020 |
| 2740 | NIRSYLNWYQHKPGKAPKLLIYAASTL | (Human) | (Human) | (Human) | (Human) | | GGNCYAGYF | | | (https://www.nature.co |
| | QSGVPSRFSGSESGTDFTLTISSLQPED | | | | | | DY | | | m/articles/s41591-020- |
| | FATYYCQQSSSSPITFGQGTKLEIK | | | | | | | | | 0998-x) |
| CoV2- | DIQMTQSPSSLSASVGDRVTITCRASQ | IGHV4-34 | IGHJ6 | IGKV1-39 | IGKJ4 | 2642 | ARVGYSQGYY | 3844 | QQSYTTLL | Seth Zost et al., 2020 |
| 2749 | SISNYLNWYQQKPGKAPKLLIYAASSL | (Human) | (Human) | (Human) | (Human) | | YYYMDV | | T | (https://www.nature.co |
| | QSGVPSRFSGSGSGTDFSLTISSLQPED | | | | | | | | | m/articles/s41591-020- |
| | FATYSCQQSYTTLLTFGGGTKVEIK | | | | | | | | | 0998-x) |
| CoV2- | SYELAQPPSVSVAPGETATIFCRATYIG | IGHV3-9 | IGHJ6 | IGLV3-21 | IGLJ2 | 2643 | ARDYCSSTTC | 3845 | QVWDGIN | Seth Zost et al., 2020 |
| 2751 | RKNVQWYQQKPGQAPVLVVYDDSD | (Human) | (Human) | (Human) | (Human) | | PAETYYYMDV | | DRVV | (https://www.nature.co |
| | RPSGIPGRFSGSNSGDTATLTISRIEAG | | | | | | | | | m/articles/s41591-020- |
| | DEAAYYCQVWDGINDRVVFGGGTKL | | | | | | | | | 0998-x) |
| | TVL | | | | | | | | | |
| CoV2- | DIQMTQSPSSLSASVGDRVTITCQASQ | IGHV3-53 | IGHJ5 | IGKV1-33 | IGKJ4 | 2644 | ARDFLRWHD | 3846 | QQYDNLP | Seth Zost et al., 2020 |
| 2752 | DINNYLNWYQQKPGKAPKLLIYDASN | (Human) | (Human) | (Human) | (Human) | | L | | PV | (https://www.nature.co |
| | LETGVPLRFSGSGSGTDFTFTISSLQPE | | | | | | | | | m/articles/s41591-020- |
| | DIATYYCQQYDNLPPVFGGGTKVEIK | | | | | | | | | 0998-x) |
| CoV2- | DFQMTQSPSSLSASVGDRVTITCRASQ | IGHV3-13 | IGHJ4 | IGKV1-39 | IGKJ1 | 2645 | ARVDFDILTG | 3847 | QQSYSSE | Seth Zost et al., 2020 |
| 2753 | SISTYLNWYQQKPGKAPNLLIFAASSLH | (Human) | (Human) | (Human) | (Human) | | YYSN | | WT | (https://www.nature.co |
| | SGVPSRFSGSGSGTDFTLSISSLQPEDF | | | | | | | | | m/articles/s41591-020- |
| | ATYYCQQSYSSEWTFGQGTKVEIK | | | | | | | | | 0998-x) |
| CoV2- | QSLLTQPPSVSGAPGQRVTLSCAGATS | IGHV3-23 | IGHJ4 | IGLV1-40 | IGLJ3 | 2646 | AKWAGPIVM | 3848 | QSYDISLG | Seth Zost et al., 2020 |
| 2756 | NIGAGSDVHWYQQLPGTAPKLLIYYN | (Human) | (Human) | (Human) | (Human) | | KYYLQY | | GWV | (https://www.nature.co |
| | TNRPSGVPDRFSGSKSATSASLVITGL | | | | | | | | | m/articles/s41591-020- |
| | QTEDEADYYCQSYDISLGGWVFGGGT | | | | | | | | | 0998-x) |
| | KLTVL | | | | | | | | | |
| CoV2 | DIQMTQSPSSLSASVGDRVTITCRASQ | IGHV3-13 | IGHJ2 | IGKV1-39 | IGKJ5 | 2647 | ARGGDSGYD | 3849 | QQSYSMP | Seth Zost et al., 2020 |
| 2758 | SVSIYLNWYQQKPGKAPKLLIYAASSL | (Human) | (Human) | (Human) | (Human) | | LGAWYFDL | | PIT | (https://www.nature.co |
| | QSGVPSRFSGSGSGTDFTLTVSSLQPE | | | | | | | | | m/articles/s41591-020- |
| | DFATYYCQQSYSMPPITFGQGTRLEIK | | | | | | | | | 0998-x) |
| CoV2- | QSVLTQPPSASGTPGQRVTISCSGSSS | IGHV5-51 | IGHJ5 | IGLV1-44 | IGLJ3 | 2648 | ARTPTLYNWF | 3850 | AAWDDSL | Seth Zost et al., 2020 |
| 2759 | NIGSNSLNWYQQLPGTAPKLLIYSNN | (Human) | (Human) | (Human) | (Human) | | HP | | NGSWV | (https://www.nature.co |
| | QRPSGVPDRFSGSKSGTSASLAISGLQ | | | | | | | | | m/articles/s41591-020- |
| | SEDEADYYCAAWDDSLNGSWVFGGG | | | | | | | | | 0998-x) |
| | TKLTVL | | | | | | | | | |
| CoV2- | QSVLTQPPSASGTPGQRVTISCSGSRY | IGHV2-70 | IGHJ6 | IGLV1-47 | IGLJ2 | 2649 | ARATTFFYGM | 3851 | AAWDDSL | Seth Zost et al., 2020 |
| 2760 | NIGSNYVYWYQQLPGTAPRLLIYSNN | (Human) | (Human) | (Human) | (Human) | | DV | | SGL | (https://www.nature.co |
| | QRPSGVPDRFSGSKSGTSASLAISGLRS | | | | | | | | | m/articles/s41591-020- |
| | EDEADYYCAAWDDSLSGLIFGGGTKLT | | | | | | | | | 0998-x) |
| | VQ | | | | | | | | | |
| CoV2- | QSALTQPASVSGSPGQSITISCTGTSSD | IGHV4-39 | IGHJ4 | IGLV2-23 | IGLJ3 | 2650 | ARHQRYCSSS | 3852 | CSYAGSST | Seth Zost et al., 2020 |
| 2762 | VGSYNLVSWYQQHPGKAPKLMIYEGS | (Human) | (Human) | (Human) | (Human) | | SCHVWDY | | WL | (https://www.nature.co |
| | KRPSGVSNRFSGSKSGNTASLTISGLQ | | | | | | | | | m/articles/s41591-020- |
| | AEDEADYYCCSYAGSSTWLFGGGTKLT | | | | | | | | | 0998-x) |
| | VL | | | | | | | | | |
| CoV2- | SYVLTQPPSVSVAPGKTARITCEGNNI | IGHV3-9 | IGHJ6 | IGLV3-21 | IGLJ2 | 2651 | AKGRGAGYTS | 3853 | QVWDSSS | Seth Zost et al., 2020 |
| 2765 | GSKSVHWYQQKPGQAPVLVVYDDSG | (Human) | (Human) | (Human) | (Human) | | YMDV | | DHHVV | (https://www.nature.co |
| | RPSGIPERFSGSNSGNTATLTISRVEAG | | | | | | | | | m/articles/s41591-020- |
| | DEADYFCQVWDSSSDHHVVFGGGTK | | | | | | | | | 0998-x) |
| | LTVL | | | | | | | | | |
| CoV2- | QSVLTQPPSVSGAPGQRVTISCTGSSS | IGHV3-53 | IGHJ3 | IGLV1-50 | IGLJ2 | 2652 | ARSYDILTGY | 3854 | VAWDDSR | Seth Zost et al., 2020 |
| 2767 | NIGAGYDVHWYQQLPETAPKLLIYAN | (Human) | (Human) | (Human) | (Human) | | RDAFDI | | NGLV | (https://www.nature.co |
| | SNRPSGVPDRFSGSKSGTSASLAISGL | | | | | | | | | m/articles/s41591-020- |
| | QSEAEADYYCVAWDDSRNGLVFGGG | | | | | | | | | 0998-x) |
| | AKLTVL | | | | | | | | | |
| CoV2- | QSVLTQPPSVSAAPGQKVTISCSGSSS | IGHV3-30- | IGHJ6 | IGLV1-51 | IGLJ1 | 2653 | ARDDNSPQG | 3855 | GTWDSSLS | Seth Zost et al., 2020 |
| 2768 | NIGNNYVSWYQQLPGTAPKLLIYDNN | 3 (Human) | (Human) | (Human) | (Human) | | SGWYFYYYYA | | AYV | (https://www.nature.co |
| | ERPSGIPDRFSGSKSGTSATLGITGLQT | | | | | | MDV | | | m/articles/s41591-020- |
| | GDEADYYCGTWDSSLSAYVFGTGTKV | | | | | | | | | 0998-x) |
| | TVL | | | | | | | | | |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV2 | SARS- | | SARS-CoV1, | S; NTD | B-cells; SARS-CoV2 | 566 | QVQLVESGGGVVQPGRSLRLSCAASGF | 1645 |
| 2769 | | | CoV1 | | SARS-CoV2 | | Human Patient | | TFSTYAMHWVRQAPGKGLEWVAVISY | |
| | | | | | | | | | DGSNKYYADSVKGRFTISRDNSKNTLYL | |
| | | | | | | | | | QMIGLRAEDTAVYYCARDWAPTYYDM | |
| | | | | | | | | | PSAFDIWGQGTMVTVSS | |
| CoV2- | Ab | SARS-CoV1 | | | SARS-CoV2 | S; non- | B-cells; SARS-CoV2 | 567 | EVQLVESGGGLVQPGGSLRLSCAASGFT | 1646 |
| 2774 | | SARS-CoV2 | | | | RBD | Human Patient | | FSSYWMTWVRQAPGKGLEWVANIKQ | |
| | | | | | | | | | DGSEKYYVDSVKGRFTISRDNAKNSLSL | |
| | | | | | | | | | QMNSLRVEDTAVYYCVRLGVSSWYFDY | |
| | | | | | | | | | WGQGTLVTVSS | |
| CoV2- | Ab | SARS-CoV1, | | | SARS-CoV2 | S; non- | B-cells; SARS-CoV2 | 568 | EVQLVESGGDLVQPGGSLRLTCAASGFT | 1647 |
| 2776 | | SARS-CoV2 | | | | RBD | Human Patient | | FSSHWMTWVRQAPGKGLEWVANIKE | |
| | | | | | | | | | DGREKYYVDSVKGRLTISRDNAKNSLYL | |
| | | | | | | | | | QMNSLRAEDTAVYYCARVVVEVATNKG | |
| | | | | | | | | | IHGVDYYYYYYMDVWGKGTTVTVSS | |
| CoV2- | Ab | SARS-CoV2 | SARS- | SARS-CoV2 | | S; RBD | B-cells; SARS-CoV2 | 569 | QVQLQQWGAGLLKPSETLSRTCAVYGA | 1648 |
| 2780 | | | CoV1 | (weak) | | | Human Patient | | SFSNYYWSWIRQPPGKGLEWIGEINHSE | |
| | | | | | | | | | NTNYNPSLKSRVTISVDTSKNQFSLRLSS | |
| | | | | | | | | | VTAADTAVYYCARLRYSSSGGHIFDYWG | |
| | | | | | | | | | QGTLVTVSS | |
| CoV2- | Ab | SARS-CoV1, | | | SARS-CoV2 | S; RBD | B-cells; SARS-CoV2 | 570 | QVQLVQSGAEVKKPGSSVKVSCKASGG | 1649 |
| 2783 | | SARS-CoV2 | | | | | Human Patient | | TFSSYAISWVRQAPGQGLEWMGGIIPIF | |
| | | | | | | | | | GTANYAQKFQGRVTITADESTSTAYMEL | |
| | | | | | | | | | SSLRSEDTAVYYCARGLTGSSAYKDEIYF | |
| | | | | | | | | | DYWGQGTLVTVSS | |
| CoV2- | Ab | SARS-CoV2 | SARS- | | SARS-CoV1, | S; non- | B-cells; SARS-CoV2 | 571 | QVQLQESGPGLVKPSETLSLTCTVSGGSI | 1650 |
| 2784 | | | CoV1 | | SARS-CoV2 | RBD | Human Patient | | SSYYWSWIRQPPGKGLEWIGYIYYSGST | |
| | | | | | | | | | KYNPSLKSRVTISVDTSKNQFSLKLSSVTA | |
| | | | | | | | | | ADTAVYYCARDGGNAYSSGWYRYYYH | |
| | | | | | | | | | MDVWGKGTTVTVSS | |
| CoV2- | Ab | SARS-CoV2 | SARS- | | SARS-CoV1, | S; RBD | B-cells; SARS-CoV2 | 572 | EVQLVESGGGLVQPGGSLRLSCAASGFT | 1651 |
| 2786 | | | CoV1 | | SARS-CoV2 | | Human Patient | | FSSYDLHWVRQGTGKRLEWVSAIGTAG | |
| | | | | | | | | | DTYYLGSVKGRFTISRENAKNSLYLQMN | |
| | | | | | | | | | SLRAGDTAVYYCARVLYDSSGFYNWFDP | |
| | | | | | | | | | WGQGTLVTVSS | |
| CoV2- | Ab | SARS-CoV2 | SARS- | | SARS-CoV1, | S; RBD | B-cells; SARS-CoV2 | 573 | QVQLVQSGAEVKKPGASVRVSCKAPGY | 1652 |
| 278 | | | CoV1 | | SARS-CoV2 | | Human Patient | | TFTTYYIHWVRQAPGQGLEWMGIINPS | |
| | | | | | | | | | AGSTTYAQKFQGRVTMTRDTSTSTVYM | |
| | | | | | | | | | ELSSLRSEDTAVYYCARGFHVPAALRNW | |
| | | | | | | | | | FDPWGQGTLVTVSS | |
| CoV2- | Ab | SARS-CoV2 | SARS- | SARS-CoV2 | | S; RBD | B-cells; SARS-CoV2 | 574 | EVQLVESGGGLVQPGGSLRLSCAASGFT | 1653 |
| 2790 | | | CoV1 | | | | Human Patient | | VSSNYMSWVRQAPGKGLEWVSVIYPG | |
| | | | | | | | | | GSAFYADSVKGRFTISRHNSNNTLCLQM | |
| | | | | | | | | | NSLRTEDTAVYYCARSYDILTGYRDAFDI | |
| | | | | | | | | | WGQGTMVTVSS | |
| CoV2- | Ab | SARS-CoV1, | | | SARS-CoV2 | S; RBD | B-cells; SARS-CoV2 | 575 | EAQLVESGGGLVQPGGSLRLSCEASGFI | 1654 |
| 2794 | | SARS-CoV2 | | | | | Human Patient | | FSSYWMSWVRQAPGKGLEWVANIKQ | |
| | | | | | | | | | DGSEKYYVDSVKGRFTISRDNAKNSLYL | |
| | | | | | | | | | QMNSLRAEDTAVYYCARVNDGRPNPLE | |
| | | | | | | | | | YYFDYWGQGTLVTVSS | |
| CoV2- | Ab | SARS-CoV2 | SARS- | | SARS-CoV1, | S; RBD | B-cells; SARS-CoV2 | 576 | QVQLQESGPGLVKPSQTLSLTCTVSGDSI | 1655 |
| 2796 | | | CoV1 | | SARS-CoV2 | | Human Patient | | SSGRYYWSWIRQHPGKGLEWIGFVYYS | |
| | | | | | | | | | GSTYYNPSLKSRVTISVDTSKNQFSLRLSS | |
| | | | | | | | | | VTAADTAVYYCARETYSAYEMPPYFDY | |
| | | | | | | | | | WGQGTLVTVSS | |
| CoV2- | Ab | SARS-CoV1, | | | SARS-CoV2 | S; RBD | B-cells; SARS-CoV2 | 577 | EVQLVQSGAEVKKPGESLKISCKGSGYSF | 1656 |
| 2797 | | SARS-CoV2 | | | | | Human Patient | | TSYWIGWVRQMPGKGLEWMGIIQPG | |
| | | | | | | | | | DSDTRYSPSFQGQVTMSADKSTSTAYLQ | |
| | | | | | | | | | WSSLKASDTAMYYCARDLIIESTIAARPG | |
| | | | | | | | | | YYGMDVWGQGTTVTVSS | |
| CoV2- | Ab | SARS-CoV2 | SARS- | | SARS-CoV1, | S; RBD | B-cells; SARS-CoV2 | 578 | QVHLVQSGAEVKKPGASVKLSCKASGYT | 1657 |
| 2801 | | | CoV1 | | SARS-CoV2 | | Human Patient | | FTNYLLHWVRQAPGQGLEWMGNVNP | |
| | | | | | | | | | SRGTATYPQKLDDRVTMTSDKSASTIYM | |
| | | | | | | | | | ELSGLRSEDTAIYYCARERSGTYFFDYWG | |
| | | | | | | | | | QGTLLTVSS | |
| CoV2- | Ab | SARS-CoV2 | SARS- | SARS-CoV2 | | S; RBD | B-cells; SARS-CoV2 | 579 | QVTLRESGPALVKPTQTLTLTCTFSGFSLT | 1658 |
| 2807 | | | CoV1 | | | | Human Patient | | TSGMCVSWIRQPPGKALEWLARIDWD | |
| | | | | | | | | | DDKYYSTSLQTRLTISKDTSKNQVVLTMT | |
| | | | | | | | | | NMDPVDTATYYCARETPVTAIDYWGQ | |
| | | | | | | | | | GTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | SYVLTQPPSVSVAPGKTARITCGGNNI | IGHV3-30- | IGHJ3 | IGLV3-21 | IGLJ3 | 2654 | ARDWAPTYY | 3856 | QVWDSSS | Seth Zost et al., 2020 |
| 2769 | GNKGVHWYQQKPGQAPVLVVDDDS | 3 | (Human) | (Human) | (Human) | | DMPSAFDI | | DHPGV | (https://www.nature.co |
| | DRPSGIPERFSGSNSGNTATLIISSVEV | (Human) | | | | | | | | m/articles/s41591-020- |
| | GDEADFYCQVWDSSSDHPGVFGGGT | | | | | | | | | 0998-x) |
| | KLTVL | | | | | | | | | |
| CoV2- | SYELTQPPSVSVSPGQTASITCSGDKLG | IGHV3-7 | IGHJ4 | IGLV3-1 | IGLJ3 | 2655 | VRLGVSSWYF | 3857 | QAWGSSR | Seth Zost et al., 2020 |
| 2774 | DKYACWYQQKPGQSPVLVIYQDTKRP | (Human) | (Human) | (Human) | (Human) | | DY | | GV | (https://www.nature.co |
| | SGIPERFSGSNSGNTATLTISGTQAMD | | | | | | | | | m/articles/s41591-020- |
| | EADYYCQAWGSSRGVFGGGTKLTVL | | | | | | | | | 0998-x) |
| CoV2- | SSELTQDPAVSVALGQTVRITCQGDSL | IGHV3-7 | IGHJ6 | IGLV3-19 | IGLJ3 | 2656 | ARVVVEVAT | 3858 | NSRDNSG | Seth Zost et al., 2020 |
| 2776 | RSYYASWYQQKPGQAPVVVIYGKNN | (Human) | (Human) | (Human) | (Human) | | NKGIHGVDYY | | NLNWV | (https://www.nature.co |
| | RPSGIPDRFSGSSSGNKASLTITGAQA | | | | | | YYYYMDV | | | m/articles/s41591-020- |
| | GDEADYYCNSRDNSGNLNWVFGGGT | | | | | | | | | 0998-x) |
| | KLTVL | | | | | | | | | |
| CoV2- | DIQMTQSPSSLSASVGDRVTITCQASQ | IGHV4-34 | IGHJ4 | IGKV1-33 | IGKJ2 | 2657 | ARLRYSSSGG | 3859 | QQYDNLPY | Seth Zost et al., 2020 |
| 2780 | DINNYLNWYQQKSGKAPKLLIYDASNL | (Human) | (Human) | (Human) | (Human) | | HIFDY | | T | (https://www.nature.co |
| | ETGVPSRFSGSGSGTDFTFTINSLQPED | | | | | | | | | m/articles/s41591-020- |
| | IATYYCQQYDNLPYTFGQGTKLEIK | | | | | | | | | 0998-x) |
| CoV2- | SYELTQPPSVSVSPGQTARITCSGDALP | IGHV1-69 | IGHJ4 | IGLV3-25 | IGLJ2 | 2658 | ARGLTGSSAY | 3860 | QSADSSGS | Seth Zost et al., 2020 |
| 2783 | KQYAYWYQQKPGQAPVLVIYKDTERP | (Human) | (Human) | (Human) | (Human) | | KDEIYFDY | | R | (https://www.nature.co |
| | SGIPERFSGSSSGTTVTLTISGVQAEDE | | | | | | | | | m/articles/s41591-020- |
| | ADYYCQSADSSGSRFGGGTKLTVL | | | | | | | | | 0998-x) |
| CoV2- | EIVMTQSPATLSVSPGERATLSCRASQ | IGHV4-59 | IGHJ6 | IGKV3-15 | IGKJ2 | 2659 | ARDGGNAYS | 3861 | QQYNNWP | Seth Zost et al., 2020 |
| 2784 | SVSSNLAWYQQKPGQAPRLLIYGAST | (Human) | (Human) | (Human) | (Human) | | SGWYRYYYH | | YT | (https://www.nature.co |
| | RATVIPARFSGSGSGTEFTLTISSLQSED | | | | | | MDV | | | m/articles/s41591-020- |
| | FAVYYCQQYNNWPYTFGQGTKLEIK | | | | | | | | | 0998-x) |
| CoV2- | DIQMTQSPSSLSASVGDRVTITCRASQ | IGHV3-13 | IGHJ5 | IGKV1-39 | IGKJ1 | 2660 | ARVLYDSSGF | 3862 | QQSYEIPP | Seth Zost et al., 2020 |
| 2786 | SISSYLNWYQQKPGKAPKLLIYAASSLE | (Human) | (Human) | (Human) | (Human) | | YNWFDP | | WT | (https://www.nature.co |
| | SGVPSRFSGSGSGTDFTLTISSLQPEDF | | | | | | | | | m/articles/s41591-020- |
| | ATYYCQQSYEIPPWTFGQGTKVEIK | | | | | | | | | 0998-x) |
| CoV2- | QSVLTQPPSASGTPGQRVTISCSGSSS | IGHV1-46 | IGHJ5 | IGLV1-44 | IGLJ2 | 2661 | ARGFHVPAAL | 3863 | ATWDDSL | Seth Zost et al., 2020 |
| 278 | NIGSNTVNWYQQLPGTAPKLLIYNNN | (Human) | (Human) | (Human) | (Human) | | RNWFDP | | NGPV | (https://www.nature.co |
| | QRPSGVPDRLSGSKSGTSASLAISGLQ | | | | | | | | | m/articles/s41591-020- |
| | SEDEADYYCATWDDSLNGPVFGGGT | | | | | | | | | 0998-x) |
| | KLTVL | | | | | | | | | |
| CoV2- | QSVLTQPPSVSGAPGQRVTISCTGSSS | IGHV3-53 | IGHJ3 | IGLV1-40 | IGLJ2 | 2662 | ARSYDILTGYR | 3864 | QSYDSRLS | Seth Zost et al., 2020 |
| 2790 | NIGSGSDVHWYQQLPGTAPKLLIYGN | (Human) | (Human) | (Human) | (Human) | | DAFDI | | GFVV | (https://www.nature.co |
| | TNRPSGVPDRFSGSKSGTSASLAITGL | | | | | | | | | m/articles/s41591-020- |
| | QAEDEADYYCQSYDSRLSGFVVFGGG | | | | | | | | | 0998-x) |
| | TKLTVL | | | | | | | | | |
| CoV2- | SYELTQPPSVSVSPGQTARITCSGDALP | IGHV3-7 | IGHJ4 | IGLV3-25 | IGLJ2 | 2663 | ARVNDGRPN | 3865 | QSADSSGT | Seth Zost et al., 2020 |
| 2794 | KQYAYWYHQKPGQAPVLVIYKDSERP | (Human) | (Human) | (Human) | (Human) | | PLEYYFDY | | SVL | (https://www.nature.co |
| | SGIPERFSGSSSGTTVTLTISGVQAEDE | | | | | | | | | m/articles/s41591-020- |
| | ADYYCQSADSSGTSVLFGGGTKLTVL | | | | | | | | | 0998-x) |
| CoV2- | QSALTQPASVSGSPGQSITISCTGTSSD | IGHV4-31 | IGHJ4 | IGLV2-23 | IGLJ3 | 2664 | ARETYSAYEM | 3866 | CSYARSS | Seth Zost et al., 2020 |
| 2796 | VGSYNLVSWYQQHPGKAPKLMIYEGS | (Human) | (Human) | (Human) | (Human) | | PPYFDY | | TRV | (https://www.nature.co |
| | KRPSGVSNRFSGSKSGNTASLTISGLQ | | | | | | | | | m/articles/s41591-020- |
| | AEDEADYYCCSYARSSTRVFGGGTKLT | | | | | | | | | 0998-x) |
| | VL | | | | | | | | | |
| CoV2- | SYELTQPPSVSVSPGQTARITCSGDAFP | IGHV5-51 | IGHJ6 | IGLV3-25 | IGLJ1 | 2665 | ARDLIIESTI | 3867 | QSADSRG | Seth Zost et al., 2020 |
| 2797 | KQYGYWYQQKPGQAPVLVIYKDSERP | (Human) | (Human) | (Human) | (Human) | | AARPGYYGM | | AV | (https://www.nature.co |
| | SGIPERFSGSSSGTTVTLTISGVQAEDE | | | | | | DV | | | m/articles/s41591-020- |
| | ADYYCQSADSRGAVFGSGTKVTVL | | | | | | | | | 0998-x) |
| CoV2- | QSVLTQPPSASGTPGQSVTISCSGSSS | IGHV1-46 | IGHJ4 | IGLV1-44 | IGLJ1 | 2666 | ARERSGTYFF | 3868 | AVWDDSL | Seth Zost et al., 2020 |
| 2801 | NIGNNKVNWYQQLPGTAPKVLIYNSN | (Human) | (Human) | (Human) | (Human) | | DY | | HSYV | (https://www.nature.co |
| | QRPSGVPDRFSGSKSGTSASLAISGLQ | | | | | | | | | m/articles/s41591-020- |
| | SEDEADYYCAVWDDSLHSYVFGTETK | | | | | | | | | 0998-x) |
| | VTVL | | | | | | | | | |
| CoV2- | DIQMTQSPSSLSASVGDRVTITCRASQ | IGHV2-70 | IGHJ4 | IGKV1-39 | IGKJ1 | 2667 | ARETPVTAID | 3869 | QQSYSTPR | Seth Zost et al., 2020 |
| 2807 | SISRYLNWYQQKPGKAPKLLIYTASSLQ | (Human) | (Human) | (Human) | (Human) | | Y | | T | (https://www.nature.co |
| | SGVPSRFSGSGSGTDFTLTISSLQPEDF | | | | | | | | | m/articles/s41591-020- |
| | ATYYCQQSYSTPRTFGQGTKVEIK | | | | | | | | | 0998-x) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV1, | | | SARS-CoV2 | S; RBD | B-cells; SARS-CoV2 | 580 | QVQLVQSGAEVKKPGSSVKVSCKASGFT | 1659 |
| 2808 | | SARS-CoV2 | | | | | Human Patient | | FMSSAVQWVRQARGQRLEWIGWIVIG | |
| | | | | | | | | | SGNTNYAQKFQERVTITRDMSTSTAYM | |
| | | | | | | | | | ELSSLRSEDTAVYYCAAPYCSSISCNDGF | |
| | | | | | | | | | DIWGQGTMVTVSS | |
| CoV2- | Ab | SARS-CoV2 | SARS- | | SARS-CoV1, | S; RBD | B-cells; SARS-CoV2 | 581 | QVQLVQSGAEVKKPGASVKVSCKVSGY | 1660 |
| 2809 | | | CoV1 | | SARS-CoV2 | | Human Patient | | TFTGYVVHWVRQAPGQDLEWMGWIN | |
| | | | | | | | | | TGYGNTKYSQKFQGRVTISWDTSATTAY | |
| | | | | | | | | | MELSNLKSEDKAVYYCASMTRMSEQTY | |
| | | | | | | | | | YGMDVWGQGTTVTVSS | |
| CoV2- | Ab | SARS-CoV1, | | | SARS-CoV2 | S; non- | B-cells; SARS-CoV2 | 582 | QVQLVESGGGVVQPGRSLRLSCAASGF | 1661 |
| 2811 | | SARS-CoV2 | | | | RBD | Human Patient | | TFSSFAMHWVRQAPGKGLEWVAVISY | |
| | | | | | | | | | DGSNKYYADSVKGRFTISRDNSKNTLYL | |
| | | | | | | | | | QMNSLRAEDTAAYYCARELMSVGWGQ | |
| | | | | | | | | | GTLVTVSS | |
| CoV2- | Ab | SARS-CoV2 | SARS- | SARS-CoV2 | | S; RBD | B-cells; SARS-CoV2 | 583 | EVQLVESGGGLIQPGGSLRLSCAASGITV | 1662 |
| 2812 | | | CoV1 | | | | Human Patient | | SSNYMSWVRQAPGKGLEWVSVIYAGG | |
| | | | | | | | | | STYYADSVKGRFTISRDNSKNTLYLQMN | |
| | | | | | | | | | SLRAEDTAVYYCARDALYYNGPGRDGM | |
| | | | | | | | | | DVWGQGTTVTVSS | |
| CoV2- | Ab | SARS-CoV2 | SARS- | SARS-CoV2 | | S; RBD | B-cells; SARS-CoV2 | 584 | EVQLVESGGGLIQPGGSLRLSCAASGITV | 1663 |
| 2813 | | | CoV1 | | | | Human Patient | | SSNYMSWVRQAPGKGLEWVSVIYAGG | |
| | | | | | | | | | STYYADSVKGRFTISRDNSKNTLYLQMN | |
| | | | | | | | | | SLRAEDTAVYYCARDALYYNGPGRDGM | |
| | | | | | | | | | DVWGQGTTVTVSS | |
| CoV2- | Ab | SARS-CoV1, | | | SARS-CoV2 | S; non- | B-cells; SARS-CoV2 | 585 | QVQLVESGGGVVQPGRSLRLSCAASGF | 1664 |
| 2814 | | SARS-CoV2 | | | | RBC | Human Patient | | TFSSYGMHWVRQAPGKGLEWVAVISS | |
| | | | | | | | | | DGSNKYYAGSVKGRFTISRDNSKNTLYL | |
| | | | | | | | | | QMNSLRAEDTAVYYCAKDMVEPLFSHY | |
| | | | | | | | | | YYYGMDVWGQGTTVTVSS | |
| CoV2- | Ab | SARS-CoV2 | SARS- | | SARS-CoV1, | S; NTD | B-cells; SARS-CoV2 | 586 | QVQLQESGPGLVKPSETLSLTCTVSGGSI | 1665 |
| 2816 | | | CoV1 | | SARS-CoV2 | | Human Patient | | SSYYWSWIRQPPGKGLEWIGYIYYSGST | |
| | | | | | | | | | NYNPSLKSRVTISVDMSKNQFSLKLRSVT | |
| | | | | | | | | | AADTAVYYCARAPRERLQWGEYYFDYW | |
| | | | | | | | | | GQGTLVTVSS | |
| CoV2- | Ab | SARS-CoV1, | | | SARS-CoV2 | S; RBD | B-cells; SARS-CoV2 | 587 | EVQLVESGGGLVQPGGSLRLSCAASGFT | 1666 |
| 2817 | | SARS-CoV2 | | | | | Human Patient | | FRSYDMHWVRQVTGKGLEWVSTIGTA | |
| | | | | | | | | | GDTYYPGSVKGRFTISRENAKNSLYLQM | |
| | | | | | | | | | NSLRAGDTAVYYCARVFETKVIRGGRYY | |
| | | | | | | | | | YYYYMDVWGKGTTVTVSS | |
| CoV2- | Ab | SARS-CoV2 | SARS- | | SARS-CoV1, | S; non- | B-cells; SARS-CoV2 | 588 | QVQLQQWGAGLLKPSETLSLTCAVYGG | 1667 |
| 2818 | | | CoV1 | | SARS-CoV2 | RBD | Human Patient | | SFSGYYWSWIRQPPGKGLEWIGEINHS | |
| | | | | | | | | | GSTNYNPSLKSRVTISVDTSKNHFSLKM | |
| | | | | | | | | | NSVTAADTAVYYCARCRQMGNFYYYYN | |
| | | | | | | | | | DVWGKGTTVTVSS | |
| CoV2- | Ab | SARS-CoV2 | SARS- | SARS-CoV2 | | S; RBD | B-cells; SARS-CoV2 | 589 | QVQLQESGPGLVKPSQTLSLTCTVSGDSI | 1668 |
| 2819 | | | CoV1 | | | | Human Patient | | SSAGYYWSWIRQHPGKGLEWIGYIYYSG | |
| | | | | | | | | | RTYYNPSLKSRVTMSVDTSKNQFSLRLRS | |
| | | | | | | | | | VTAADTAVYYCARVVPTRGPVAWFDP | |
| | | | | | | | | | WGQGTLVTVSS | |
| CoV2- | Ab | SARS-CoV1, | | | SARS-CoV2 | S; non- | B-cells; SARS-CoV2 | 590 | QVQLVESGGGVVQPGRSLRLSCAASGF | 1669 |
| 2820 | | SARS-CoV2 | | | | RBD | Human Patient | | TFSSYGMHWVRQAPGKGLEWVAVIWY | |
| | | | | | | | | | DGSKKDYADSVKGRFTISRDNSKNTLYL | |
| | | | | | | | | | QMNSLRAEDTAVYYCARDQSQGAYILT | |
| | | | | | | | | | GYRGYGMDVWGQGTTVTVSS | |
| CoV2- | Ab | SARS-CoV1, | | | SARS-CoV2 | S; RBD | B-cells; SARS-CoV2 | 591 | QVQLVQSGAEVKKPGSSVKVSCKTAGG | 1670 |
| 2821 | | SARS-CoV2 | | | | | Human Patient | | TSSSYAISWVRQAPGQGLEWMGRIIPIL | |
| | | | | | | | | | GVAIYAQKFQGRVTITADKSTSTAYMEL | |
| | | | | | | | | | NSLRSEDTAVYYCTTTQGGDYGDNLYYL | |
| | | | | | | | | | DYWGQGTLVTVSS | |
| CoV2- | Ab | SARS-CoV2 | SARS- | | SARS-CoV1, | S; RBD | B-cells; SARS-CoV2 | 592 | EVQLVESGGGLVQPGGSLRLSCAASGVT | 1671 |
| 2822 | | | CoV1 | | SARS-CoV2 | | Human Patient | | VSSNYMSWVRQAPGKGLEWVSLIYSGG | |
| | | | | | | | | | STFYADSVKGRFTISRHNSKNTLYLQMN | |
| | | | | | | | | | SLRPEDTAVYYCARGPEPDAFDIWGQG | |
| | | | | | | | | | TMVTVSS | |
| CoV2- | Ab | SARS-CoV2 | SARS- | | SARS-CoV1, | S; RBD | B-cells; SARS-CoV2 | 593 | QVQLVQSGAEVKKPGASVKVSCKASGD | 1672 |
| 2826 | | | CoV1 | | SARS-CoV2 | | Human Patient | | TFTTYYIHWVRQAPGQGLEWMGIINPS | |
| | | | | | | | | | GGSRSYAQKFQGRISMTSDTSTSTVYME | |
| | | | | | | | | | LSSLRSEDTAVYYCARGYGFVPNVLYYFD | |
| | | | | | | | | | YWGQGTLVTVST | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | DIQMTQSPSSLSASVGDRVTITCRASQ | IGHV1-58 | IGHJ3 | IGKV1-33 | IGKJ3 | 2668 | AAPYCSSISCN | 3870 | QQYDNLPL | Seth Zost et al., 2020 |
| 2808 | SISNYLNWYQQKPGKAPKLLIYDASNL | (Human) | (Human) | (Human) | (Human) | | DGFDI | | A | (https://www.nature.co |
| | ETGVPSRFSGSGSGTDFTFTISSLQPDD | | | | | | | | | m/articles/s41591-020- |
| | IATYYCQQYDNLPLAFGPGTKVDIK | | | | | | | | | 0998-x) |
| CoV2- | DIQMTQSPSSLSASVGDRVTITCRASQ | IGHV1-3 | IGHJ6 | IGKV1-39 | IGKJ4 | 2669 | ASMTRMSEQ | 3871 | QQSYTTLL | Seth Zost et al., 2020 |
| 2809 | SISNYLNWYQQKPGKAPKLLIYAASSL | (Human) | (Human) | (Human) | (Human) | | TYYGMDV | | T | (https://www.nature.co |
| | QSGVPSRFSGSGSGTDFSLTISSLQPED | | | | | | | | | m/articles/s41591-020- |
| | FATYSCQQSYTTLLTFGGGTKVEIK | | | | | | | | | 0998-x) |
| CoV2- | QSVLTQPPSVSGAPGQRVTISCTGSSS | IGHV3-30 | IGHJ4 | IGLV1-40 | IGLJ3 | 2670 | ARELMSVG | 3872 | QSYDSSLS | Seth Zost et al., 2020 |
| 2811 | NIGARYDVHWYQQLPGTAPKLLMYG | (Human) | (Human) | (Human) | (Human) | | | | GWV | (https://www.nature.co |
| | NNNRPSGVPDRFSGSKSGTSVSLAITG | | | | | | | | | m/articles/s41591-020- |
| | LQAEDEADYYCQSYDSSLSGWVFGGG | | | | | | | | | 0998-x) |
| | TKLTVL | | | | | | | | | |
| CoV2- | VIQMTQSPSSLSASVGDRVTITCQASQ | IGHV3-53 | IGHJ6 | IGKV1-33 | IGKJ3 | 2671 | ARDALYYNGP | 3873 | QQYANLPF | Seth Zost et al., 2020 |
| 2812 | DINKYLNWYQQKPGKAPKLLIYDASNL | (Human) | (Human) | (Human) | (Human) | | GRDGMDV | | T | (https://www.nature.co |
| | ETGVPSRFSGSGSGTDFTFTISSLQPED | | | | | | | | | m/articles/s41591-020- |
| | IATYYCQQYANLPFTFGPGTKVDIK | | | | | | | | | 0998-x) |
| CoV2- | VIQMTQSPSSLSASVGDRVTITCQASQ | IGHV3-53 | IGHJ6 | IGKV1-33 | IGKJ2 | 2672 | ARDALYYNGP | 3874 | QQYDNLP | Seth Zost et al., 2020 |
| 2813 | DINKYLNWYQQKPGKAPKLLIYDASNL | (Human) | (Human) | (Human) | (Human) | | GRDGMDV | | RT | (https://www.nature.co |
| | ETGVPSRFSGSGSGTDFTFTISSLQPED | | | | | | | | | m/articles/s41591-020- |
| | IATYYCQQYDNLPRTFGQGTKLEIK | | | | | | | | | 0998-x) |
| CoV2- | QSVLTQPPSVSGAPGQRVTISCTGSSS | IGHV3-30 | IGHJ6 | IGLV1-40 | IGLJ1 | 2673 | AKDMVEPLFS | 3875 | QSYDSSLS | Seth Zost et al., 2020 |
| 2814 | NIGAGYDVHWYQQLPGTAPKLLIYGN | (Human) | (Human) | (Human) | (Human) | | HYYYYGMDV | | GYV | (https://www.nature.co |
| | SNRPSGVPDRFSGSKSGTSASLAISGL | | | | | | | | | m/articles/s41591-020- |
| | QAEDEADYSCQSYDSSLSGYVFGTGTK | | | | | | | | | 0998-x) |
| | VTVL | | | | | | | | | |
| CoV2- | QSALTQPASVSGSPGQSITISCTGTSSD | IGHV4-59 | IGHJ4 | IGLV2-23 | IGLJ2 | 2674 | ARAPRERLQ | 3876 | CSYAVST | Seth Zost et al., 2020 |
| 2816 | VGSYNLVSWYQQHAGKAPKLMIYEVI | (Human) | (Human) | (Human) | (Human) | | WGEYYFDY | | TYVI | (https://www.nature.co |
| | KRPSGVSNRFSGSKSGNTASLTISGLQ | | | | | | | | | m/articles/s41591-020- |
| | AEDEADYYCCSYAVSTTYVIFGGGTKLT | | | | | | | | | 0998-x) |
| | VL | | | | | | | | | |
| CoV2- | DIQMTQSPSSLSASVGDRVTITCRASQ | IGHV3-13 | IGHJ6 | IGKV1-39 | IGKJ2 | 2675 | ARVFETKVIR | 3877 | QQSYSNPS | Seth Zost et al., 2020 |
| 2817 | SISSYLNWYQQKPGKAPKLLIYAASSLQ | (Human) | (Human) | (Human) | (Human) | | GGRYYYYYYM | | YT | (https://www.nature.co |
| | SGVPSRFSGSGSGTDFTLTISSLQPEDF | | | | | | DV | | | m/articles/s41591-020- |
| | ATYYCQQSYSNPSYTFGQGTKLEIK | | | | | | | | | 0998-x) |
| CoV2- | EIVLTQSPGTLSLSPGERATLSCRASQS | IGHV4-34 | IGHJ6 | IGKV3-20 | IGKJ2 | 2676 | ARCRQMGNF | 3878 | QQYGSSPP | Seth Zost et al., 2020 |
| 2818 | VASSYLAWYQQKPGQAPRLLIYGASG | (Human) | (Human) | (Human) | (Human) | | YYYYMDV | | RYT | (https://www.nature.co |
| | RATGIPDRFSGSGSGTDFTLTISRLEPE | | | | | | | | | m/articles/s41591-020- |
| | DFAVYYCQQYGSSPPRYTFGQGTKLEI | | | | | | | | | 0998-x) |
| | K | | | | | | | | | |
| CoV2- | DIQMTQSPSSLSASVGDRVTITCRASQ | IGHV4-31 | IGHJ5 | IGKV1-39 | IGKJ2 | 2677 | ARVVPTRGPV | 3879 | QQSYSTLL | Seth Zost et al., 2020 |
| 2819 | SISSYLNWYQQRPGKAPKLLIYAASSLQ | (Human) | (Human) | (Human) | (Human) | | AWFDP | | YT | (https://www.nature.co |
| | SGVPSRFSGSGSGTDFTLTISTLQPEDF | | | | | | | | | m/articles/s41591-020- |
| | ATYYCQQSYSTLLYTFGQGTKLEIK | | | | | | | | | 0998-x) |
| CoV2- | DIVMITQSPLSLPVTPGEPASISCRSSQS | IGHV3-33 | IGHJ6 | IGKV2-28 | IGKJ3 | 2678 | ARDQSQGAYI | 3880 | MQALQTP | Seth Zost et al., 2020 |
| 2820 | LLHSNGYNYLDWYLQKPGQSPQFLIYL | (Human) | (Human) | (Human) | (Human) | | LTGYRGYGM | | FT | (https://www.nature.co |
| | GSNRASGVPDRFSGSGSGTDFILKISR | | | | | | DV | | | m/articles/s41591-020- |
| | VEAEDVGVYYCMQALQTPFTFGPGTK | | | | | | | | | 0998-x) |
| | VDIK | | | | | | | | | |
| CoV2- | SYVLTQPPSVSVAPGKTASITCEGNNI | IGHV1-69 | IGHJ4 | IGLV3-21 | IGLJ1 | 2679 | TTTQGGDYG | 3881 | QVWDSSS | Seth Zost et al., 2020 |
| 2821 | GSKSVHWYQQKPGQAPVLVIYYDSDR | (Human) | (Human) | (Human) | (Human) | | DNLYYLDY | | DRLYV | (https://www.nature.co |
| | PSGIPERFSGSNSGNTATLTISRVEAGD | | | | | | | | | m/articles/s41591-020- |
| | EADYYCQVWDSSSDRLYVFGTGTKVT | | | | | | | | | 0998-x) |
| | VL | | | | | | | | | |
| CoV2- | DIQLTQSPSFLSASVGDRVTITCRASQG | IGHV3-53 | IGHJ3 | IGKV1-9 | IGKJ2 | 2680 | ARGPEPDAFD | 3882 | QQSYSNPS | Seth Zost et al., 2020 |
| 2822 | ISSYLAWYQQKPGKAPKLLIYAASTLQS | (Human) | (Human) | (Human) | (Human) | | I | | YT | (https://www.nature.co |
| | GVPSRFSGSGSGTDFTLTISSLQPEDFA | | | | | | | | | m/articles/s41591-020- |
| | TYYCQQSYSNPSYTFGQGTKLEIK | | | | | | | | | 0998-x) |
| CoV2- | QSALTQPASVSGSPGQSITISCTGTSSD | IGHV1-46 | IGHJ4 | IGLV2-23 | IGLJ3 | 2681 | ARGYGFVPN | 3883 | CSYAGSST | Seth Zost et al., 2020 |
| 2826 | VGSYNLVSWYQQHPGKAPKLMIYEGS | (Human) | (Human) | (Human) | (Human) | | VLYYFDY | | WL | (https://www.nature.co |
| | KRPSGVSNRFSGSKSGITASLTISGLQA | | | | | | | | | m/articles/s41591-020- |
| | EDEADYYCCSYAGSSTWLFGGGTKLTV | | | | | | | | | 0998-x) |
| | L | | | | | | | | | |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV1, | | SARS-CoV2 | | S; RBD | B-cells; SARS-CoV2 | 594 | EVQLVESGGGLVKPGGSLRLSCAASGFT | 1673 |
| 2828 | | SARS-CoV2 | | | | | Human Patient | | FRDYSMNWVRQAPGKGLEWVSSISSG | |
| | | | | | | | | | GSYIYYADSVKGRFTISRDNAKNSLYLQM | |
| | | | | | | | | | NSLRAEDTAVYYCARGGSILWWLIDYW | |
| | | | | | | | | | GQGTLVTVSS | |
| CoV2- | Ab | SARS-CoV2 | SARS- | | SARS-CoV1, | S; NTD | B-cells; SARS-CoV2 | 595 | EVQLLESGGGLVQPGGSLRLSCAASGFT | 1674 |
| 2830 | | | CoV1 | | SARS-CoV2 | | Human Patient | | FTSYAMSWVRQAPGKGLEWVSGISISG | |
| | | | | | | | | | GSTYYAASVKGRFTISRDNSKNTLYLQM | |
| | | | | | | | | | NSLRAEDTAVYYCAKDSRSGIAGVDAFD | |
| | | | | | | | | | IWGQGTMVTVSS | |
| CoV2- | Ab | SARS-CoV2 | SARS- | SARS-CoV2 | | S; RBD | B-cells; SARS-CoV2 | 596 | EVQLVESGGGLVQPGGSLRLSCAASGLT | 1675 |
| 2832 | | | CoV1 | | | | Human Patient | | VSSNYMSWVRQAPGKGLECVSVIYAGG | |
| | | | | | | | | | NTYYADSVKGRFTISRDNSKNTLYLQMN | |
| | | | | | | | | | SLRAEDTAVYYCARGDGGYYSPFDYWG | |
| | | | | | | | | | QGTLVTVSS | |
| CoV2- | Ab | SARS-CoV1, | | | SARS-CoV2 | S; RBD | B-cells; SARS-CoV2 | 597 | QITLKESGPTLVKPTQTLTLTCTFSGFSLS | 1676 |
| 2834 | | SARS-CoV2 | | | | | Human Patient | | TSGVGVAWIRQPPGKALEWLALIYWDD | |
| | | | | | | | | | DKRYSPSLKSRLTITKDTSKNQVVLTMTN | |
| | | | | | | | | | MDPVDTATYYCAHRLPTPQLLPSFENW | |
| | | | | | | | | | FDPWGQGTLVTVSS | |
| CoV2- | Ab | SARS-CoV2 | SARS- | SARS-CoV2 | | S; RBD | B-cells; SARS-CoV2 | 598 | EVQLVESGGGLVQPGGSLRLSCAASGLT | 1677 |
| 2835 | | | CoV1 | | | | Human Patient | | VGSNYMNWVRQAPGKGLEWVSVIYSG | |
| | | | | | | | | | GSTFYADSVKGRFTISRDNSKNTLYLQLN | |
| | | | | | | | | | SLRAEDTAVYYCAREVVGYFDCWGQGT | |
| | | | | | | | | | LVTVSS | |
| CoV2- | Ab | SARS-CoV2 | SARS- | SARS-CoV2 | | S; RBD | B-cells; SARS-CoV2 | 599 | QVQLQESGPGLVKPSETLSLTCTVSGGSI | 1678 |
| 2841 | | | CoV1 | | | | Human Patient | | SSYYWSWIRQPPGKGLEWIGHIYYTGSS | |
| | | | | | | | | | YYNPSLKSRVTISLDTSKNQFSLKLNSVTA | |
| | | | | | | | | | ADTAVYYCARLRWLRGGIDFWGQGTLV | |
| | | | | | | | | | IVSS | |
| CoV2- | Ab | SARS-CoV1, | | | SARS-CoV2 | S; RBD | B-cells; SARS-CoV2 | 600 | QVQLVQSGAEVKKPGASVKVSCKASGD | 1679 |
| 2842 | | SARS-CoV2 | | | | | Human Patient | | TFTTYYIHWVRQAPGQGLEWMGIINPS | |
| | | | | | | | | | GGSRSYAQKFQGRISMTSDTSTSTVYME | |
| | | | | | | | | | LSSLRSEDTAVYYCARGYGFVPNVLYYFD | |
| | | | | | | | | | YWGQGILVTVSS | |
| CoV2- | Ab | SARS-CoV1, | | | SARS-CoV2 | S; non- | B-cells; SARS-CoV2 | 601 | QVELVESGGGVVQPGRSLRLSCAASGFI | 1680 |
| 2844 | | SARS-CoV2 | | | | RBD | Human Patient | | FSSYAMHWVRQAPGKGLEWVAVISYD | |
| | | | | | | | | | GGNKYYADSVKGRFTISRDNSKNTLYLQ | |
| | | | | | | | | | MNSLRAEDTAVYYCARAQGGNYYYGM | |
| | | | | | | | | | DVWGQGTTVTVSS | |
| CoV2- | Ab | SARS-CoV1, | | SARS-CoV2 | | S; RBD | B-cells; SARS-CoV2 | 602 | QVQLVESGGGVVQPGRSLRLSCAASGF | 1681 |
| 2848 | | SARS-CoV2 | | (weak) | | | Human Patient | | TFSRYAMYWVRQAPGKGLEWVALISYD | |
| | | | | | | | | | GRNEYYADSVKGRFTISRDNSKNTLYLQ | |
| | | | | | | | | | MNSLRAGDSAVYYCARDLAYHPYRDYG | |
| | | | | | | | | | DDDYYYYYGMDVWGQGTTVTVSS | |
| CoV2- | Ab | SARS-CoV2 | SARS- | | SARS-CoV1, | S; RBD | B-cells; SARS-CoV2 | 603 | QVQLVQSGAEVKKPGASVKVSCKASGY | 1682 |
| 2853 | | | CoV1 | | SARS-CoV2 | | Human Patient | | TFTSYYMHWVRQAPGQGLEWMGIINP | |
| | | | | | | | | | GGGSTTYAQKFQGRVTMTSDTSTSTVY | |
| | | | | | | | | | MELSSLRSEDTAMYYCARGAIPPNSRAE | |
| | | | | | | | | | IDYWGQGTLVTVSS | |
| CoV2- | Ab | SARS-CoV1, | | | SARS-CoV2 | S; RBD | B-cells; SARS-CoV2 | 604 | QVQLVQSGAEVKKPGASVKVSCKASGY | 1683 |
| 2863 | | SARS-CoV2 | | | | | Human Patient | | TFTSYYLHWVRQAPGQGLEWMGIINPS | |
| | | | | | | | | | GGSTTYAQKFQGRVTMTRDTSTSAVYM | |
| | | | | | | | | | ELRSLRSEDTAVYYCARENDYGDYVEPR | |
| | | | | | | | | | DYYYGMDVWGQGTTVTVSS | |
| CoV2- | Ab | SARS-CoV2 | SARS- | | SARS-CoV1, | S; non- | B-cells; SARS-CoV2 | 605 | QVQLVESGGGVVQPGRSLRLSCAASGF | 1684 |
| 2872 | | | CoV1 | | SARS-CoV2 | RBD | Human Patient | | TFSTYGMHWVRQAPGKGLEWVAVIWY | |
| | | | | | | | | | DGNKKYCADSVKGRCTISRDNSKNTLYL | |
| | | | | | | | | | QMNSLRAEDTAVYYCAREGPFGDREAS | |
| | | | | | | | | | GAFDVWGQGTMVTVSS | |
| CoV2- | Ab | SARS-CoV1, | | | SARS-CoV2 | S; RBD | B-cells; SARS-CoV2 | 606 | QVQLVESGGGLVKPGGSLRLSCAASGFT | 1685 |
| 2873 | | SARS-CoV2 | | | | | Human Patient | | FSDYYMSWIRQAPGKGLEWVSYISSSGI | |
| | | | | | | | | | TIYYADSVKGRFTISRDNAKNSLYLQMNS | |
| | | | | | | | | | LRAEDTAVYYCTGVVAAPAEYFQHWGQ | |
| | | | | | | | | | GTLVTVSS | |
| CoV2- | Ab | SARS-CoV1, | | SARS-CoV2 | | S; RBD | B-cells; SARS-CoV2 | 607 | EVQLVESGGGLVQPGGSLRLSCAASGFT | 1686 |
| 2878 | | SARS-CoV2 | | (weak) | | | Human Patient | | FSRHWMTWVRQAPGKGLEWVANIKQ | |
| | | | | | | | | | DGSEKYYVDSVKGRLTISRDNAKNSLYL | |
| | | | | | | | | | QMNSLRAEDTAVYYCARLGFYYGGADY | |
| | | | | | | | | | WGQGTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | NFMLTQPHSVSESPGKTVTISCTGSSG | IGHV3-21 | IGHJ4 | IGLV6-57 | IGLJ2 | 2682 | ARGGSILWW | 3884 | QSYDSTSR | Seth Zost et al., 2020 |
| 2828 | SIASNYVQWYQQRPGSAPTTVIYEDN | (Human) | (Human) | (Human) | (Human) | | LIDY | | VV | (https://www.nature.co |
| | QRPSGVPDRFSGSIDSSSNSASLTISGL | | | | | | | | | m/articles/s41591-020- |
| | KTEDEADYYCQSYDSTSRVVFGGGTKL | | | | | | | | | 0998-x) |
| | TVL | | | | | | | | | |
| CoV2- | SYVLTQPPSVSVAPGRTARITCGGNNI | IGHV3-23 | IGHJ3 | IGLV3-21 | IGLJ2 | 2683 | AKDSRSGIAG | 3885 | QVWDSGS | Seth Zost et al., 2020 |
| 2830 | GSKSVHWYQQKPGQAPVLVVYDDSD | (Human) | (Human) | (Human) | (Human) | | VDAFDI | | DHVV | (https://www.nature.co |
| | RPSGIPERFSGSKFGNTATLIISRVEAG | | | | | | | | | m/articles/s41591-020- |
| | DEADYSCQVWDSGSDHVVFGGGTKV | | | | | | | | | 0998-x) |
| | TVL | | | | | | | | | |
| CoV2- | DIQMTQSPSSLSASVGDRVTITCRASQ | IGHV3-66 | IGHJ4 | IGKV1-39 | IGKJ1 | 2684 | ARGDGGYYS | 3886 | QQSYSTPQ | Seth Zost et al., 2020 |
| 2832 | SISSYLNWYQQKPGKAPKVLIYAASTM | (Human) | (Human) | (Human) | (Human) | | PFDY | | T | (https://www.nature.co |
| | QSGVPSRFRGSGSGTDFTLTISSLQLED | | | | | | | | | m/articles/s41591-020- |
| | FATYYCQQSYSTPQTFGQGTKVEIK | | | | | | | | | 0998-x) |
| CoV2- | QSVLTQPPSVSEAPRQRVTISCSGGSS | IGHV2-5 | IGHJ5 | IGLV1-36 | IGLJ2 | 2685 | AHRLPTPQLL | 3887 | ASWDDSLI | Seth Zost et al., 2020 |
| 2834 | NIGNNAVNWYQQLPGKAPKLLIYYDD | (Human) | (Human) | (Human) | (Human) | | PSFENWFDP | | GPV | (https://www.nature.co |
| | LLPSGVSDRFSGSKSGTSASLAISGLQS | | | | | | | | | m/articles/s41591-020- |
| | EDEADYYCASWDDSLIGPVFGGGTKL | | | | | | | | | 0998-x) |
| | TVL | | | | | | | | | |
| CoV2- | DIQLTQSPSFLSASVGDRVTITCRASQG | IGHV3-66 | IGHJ4 | IGKV1-9 | IGKJ2 | 2686 | AREVVGYF | 3888 | QQLNSYPG | Seth Zost et al., 2020 |
| 2835 | ISTYLAWYQQKPGKAPKLLIYAASTLQS | (Human) | (Human) | (Human) | (Human) | | DC | | YT | (https://www.nature.co |
| | GVPSRFSGSGSGTEFTLTISSLQPEDFA | | | | | | | | | m/articles/s41591-020- |
| | TYYCQQLNSYPGYTFGQGTKLEIK | | | | | | | | | 0998-x) |
| CoV2- | NLMLTQPHSVSESPGKTVTISCTGSSG | IGHV4-59 | IGHJ4 | IGLV6-57 | IGLJ3 | 2687 | ARLRWLRG | 3889 | QSYDSNN | Seth Zost et al., 2020 |
| 2841 | SIASNYVQWYQQRPGSAPTTVIYEDN | (Human) | (Human) | (Human) | (Human) | | GIDF | | QV | (https://www.nature.co |
| | QRPSGVPDRFSGSIDSSSNSASLTISGL | | | | | | | | | m/articles/s41591-020- |
| | KTEDEADYYCQSYDSNNQVFGGGTKL | | | | | | | | | 0998-x) |
| | TVL | | | | | | | | | |
| CoV2- | NFMLTQPHSVSESPGKTVTISCTRSSG | IGHV1-46 | IGHJ4 | IGLV6-57 | IGLJ2 | 2688 | ARGYGFVPN | 3890 | QSYDSSDV | Seth Zost et al., 2020 |
| 2842 | SIASSYVQWYQQRPGSAPTTVIYEDN | (Human) | (Human) | (Human) | (Human) | | VLYYFDY | | V | (https://www.nature.co |
| | QRPSGVPDRFSGSIDSSSNSASLTISGL | | | | | | | | | m/articles/s41591-020- |
| | KTEDEADYYCQSYDSSDVVFGGGTKLT | | | | | | | | | 0998-x) |
| | VL | | | | | | | | | |
| CoV2- | SYVLTQPPSVSVAPGKTARITCGGNNI | IGHV3-30- | IGHJ6 | IGLV3-21 | IGLJ2 | 2689 | ARAQGGNYY | 3891 | QVWDSSS | Seth Zost et al., 2020 |
| 2844 | GSKNVHWYQQKPGQAPVKVVYHDG | 3 | (Human) | (Human) | (Human) | | YGMDV | | DHHVV | (https://www.nature.co |
| | DRPSGIPERFSGSNSGNTATLTINRVE | (Human) | | | | | | | | m/articles/s41591-020- |
| | AGDEADYSCQVWDSSSDHHVVFGGG | | | | | | | | | 0998-x) |
| | TKLTVL | | | | | | | | | |
| CoV2- | SYELTQPPSVSVSPGQTARITCFGDALP | IGHV3-30 | IGHJ6 | IGLV3-25 | IGLJ3 | 2690 | ARDLAYHPY | 3892 | QSADSSGT | Seth Zost et al., 2020 |
| 2848 | KQYAYWYQQKPGQGPVLVIYKDSERP | (Human) | (Human) | (Human) | (Human) | | RDYGDDDYY | | YRV | (https://www.nature.co |
| | SGIPERFSGSTSGTTVTLTISGVQAEDE | | | | | | YYYGMDV | | | m/articles/s41591-020- |
| | ADYYCQSADSSGTYRVFGGGTKLTVL | | | | | | | | | 0998-x) |
| CoV2- | EIVMTQSPATLSVSPGERVTLSCRASQ | IGHV1-46 | IGHJ4 | IGKV3-15 | IGKJ4 | 2691 | SRARGAIP | 3893 | QQYYNWP | Seth Zost et al., 2020 |
| 2853 | SVSSNLAWCQQKPGQAPRLLIYGAST | (Human) | (Human) | (Human) | (Human) | | PNAEIDY | | LT | (https://www.nature.co |
| | RATGIPARFSGSGSGTEFTLTISSLQSE | | | | | | | | | m/articles/s41591-020- |
| | DFAVYYCQQYYNWPLTFGGGTKVEIK | | | | | | | | | 0998-x) |
| CoV2- | SYELTQPPSVSVSPGQTARITCSGDALP | IGHV1-46 | IGHJ6 | IGLV3-25 | IGLJ2 | 2692 | ARENDYGDY | 3894 | QSADSSAA | Seth Zost et al., 2020 |
| 2863 | KQYAYWYQQKPGQAPVLVIYKDSERP | (Human) | (Human) | (Human) | (Human) | | VEPRDYYYG | | YVV | (https://www.nature.co |
| | SGIPERFSGSSSGTTVTLTISGVQAEDE | | | | | | MDV | | | m/articles/s41591-020- |
| | ADYYCQSADSSAAYVVFGGGTKLTVL | | | | | | | | | 0998-x) |
| CoV2- | SYELTQPPSVSVSPGQTARITCSGDALP | IGHV3-33 | IGHJ3 | IGLV3-10 | IGLJ3 | 2693 | AREGPFGD | 3895 | YSTDSSGK | Seth Zost et al., 2020 |
| 2872 | KKYTYWYQQKSGQAPVVVIYEDSKRP | (Human) | (Human) | (Human) | (Human) | | REASGAFDV | | GV | (https://www.nature.co |
| | SGIPERFSGSSSGTMATLTISGAQVED | | | | | | | | | m/articles/s41591-020- |
| | EADYYCYSTDSSGKGVFGGGTKLTVL | | | | | | | | | 0998-x) |
| CoV2- | SYVLTQPPSVSVAPGKTARITCGGNNI | IGHV3-11 | IGHJ1 | IGLV3-21 | IGLJ1 | 2694 | TGVVAAPA | 3896 | QVWDSSS | Seth Zost et al., 2020 |
| 2873 | GSKSVHWYQQKPGQAPVLVVYDDSD | (Human) | (Human) | (Human) | (Human) | | EYFQI | | DPFYV | (https://www.nature.co |
| | RPSGIPERFSGSNSGNTATLTISRVEAG | | | | | | | | | m/articles/s41591-020- |
| | DEADYYCQVWDSSSDPFYVFGTGTKV | | | | | | | | | 0998-x) |
| | TVL | | | | | | | | | |
| CoV2- | NFMLTQPHSVSESPGKTVTISCTGSSG | IGHV3-7 | IGHJ4 | IGLV6-57 | IGLJ3 | 2695 | ARLGFYYG | 3897 | QSYDGINR | Seth Zost et al., 2020 |
| 2878 | SIASNYVQWYQQRPGSAPTTVISEDN | (Human) | (Human) | (Human) | (Human) | | GADY | | AWV | (https://www.nature.co |
| | QRPSGVPDRFSGSIDSSSNSASLTISGL | | | | | | | | | m/articles/s41591-020- |
| | KTEDEADYYCQSYDGINRAWVFGGGT | | | | | | | | | 0998-x) |
| | KLTVL | | | | | | | | | |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV1, | | | SARS-CoV2 | S; non- | B-cells; SARS-CoV2 | 608 | QVQLVESGGGVVQPGRSLRLSCAASGF | 1687 |
| 2883 | | SARS-CoV2 | | | | RBD | Human Patient | | TFSTYGMHWVRQAPGKGLEWVAVISY | |
| | | | | | | | | | DGSNKYYADSVKGRFTISRDNSKNTLYL | |
| | | | | | | | | | QMNSLRAEDTAMYYCAKDGSIAAADY | |
| | | | | | | | | | WGQGTLVTVSS | |
| CoV2- | Ab | SARS-CoV2 | SARS- | | SARS-CoV1, | S; RBD | B-cells; SARS-CoV2 | 609 | QVQLVQAGAEVKKPGASVKVSCKASGY | 1688 |
| 2891 | | | CoV1 | | SARS-CoV2 | | Human Patient | | TFTSYYMHWVRQAPGQGLEWMGIINP | |
| | | | | | | | | | SGGSTSYAQKFQGRVTMTRDTSTSTVY | |
| | | | | | | | | | MELSSLRSEDTAVYYCARGAAVPAAGEF | |
| | | | | | | | | | DYWGQGTLVTVSS | |
| CoV2- | Ab | SARS-CoV1, | | | SARS-CoV2 | S; RBD | B-cells; SARS-CoV2 | 610 | QVQLVESGGGVVQSGGSLRLSCAASGF | 1689 |
| 2894 | | SARS-CoV2 | | | | | Human Patient | | TFSTYGMHWVRQAPGKGLEWVAIISYD | |
| | | | | | | | | | GINKYYADSVKGRFTISRDNSKNTVYLQ | |
| | | | | | | | | | MNSLRTEDTAMYYCAKGDGSYLMDYF | |
| | | | | | | | | | DYWGQGTLVTVSS | |
| CoV2- | Ab | SARS-CoV2 | SARS- | | SARS-CoV1, | S; RBD | B-cells; SARS-CoV2 | 611 | EVQLVESGGGLVQPGGSLRLSCAASGFT | 1690 |
| 2901 | | | CoV1 | | SARS-CoV2 | | Human Patient | | VSSNYMSWVRQAPGKGLEWVSVIYPG | |
| | | | | | | | | | GSAFYADSVKGRFTISRHNSNNTLCLQM | |
| | | | | | | | | | NSLRTEDTAVYYCARSYDILTGYRDAFDI | |
| | | | | | | | | | WGQGTMVTVSS | |
| CoV2- | Ab | SARS-CoV1, | | | SARS-CoV2 | S; non- | B-cells; SARS-CoV2 | 612 | QVQLQQWGAGLLKPSETLSLTCAVYGG | 1691 |
| 2904 | | SARS-CoV2 | | | | RBC | Human Patient | | SFSGYYWSWIRQPPGKGLEWIGEINHS | |
| | | | | | | | | | GSTNYNPSLKSRVTISVDTSKNHFSLKM | |
| | | | | | | | | | NSVTAADTAVYYCARCRQMGNFYYYYM | |
| | | | | | | | | | DVWGKGTTVTVSP | |
| CoV2- | Ab | SARS-CoV1, | | | SARS-CoV2 | S; RBD | B-cells; SARS-CoV2 | 513 | EVQLVQSGAEVKKPGESLKISCKGSGYSF | 1692 |
| 2906 | | SARS-CoV2 | | | | | Human Patient | | TSYWVGWVRQMPGKGLEWMGIIYPG | |
| | | | | | | | | | DSDTRDSPSFQGQVTISADKSISTAYLQ | |
| | | | | | | | | | WSSLKASDTAMYYCARLGSESKIDYYYY | |
| | | | | | | | | | GMDVWGQGTTVTVSS | |
| CoV2- | Ab | SARS-CoV2 | SARS- | | SARS-CoV1, | S; RBD | B-cells; SARS-CoV2 | 614 | QVQLVQSGAEVKKPGSSVKVSCKASGD | 1693 |
| 2909 | | | CoV1 | | SARS-CoV2 | | Human Patient | | TFSSYTINWVRQAPGQGLEWMGRIIPIL | |
| | | | | | | | | | GIPNYAQKFQGRVTITADKSTSTAFMEL | |
| | | | | | | | | | SSLRSEDTAVYYCARGRGYSNYGASYYM | |
| | | | | | | | | | DVWGKGTTVTVSS | |
| CoV2- | Ab | SARS-CoV2 | SARS- | | SARS-CoV1, | S; RBD | B-cells; SARS-CoV2 | 615 | QLQLRESGPGLVKPSETLSLTCTVSGGSI | 1694 |
| 2911 | | | CoV1 | | SARS-CoV2 | | Human Patient | | SSSSYYWGWIRQPPGKGLEWIGSIYYSG | |
| | | | | | | | | | STYYNPSLKSRVTISVDTSKNQFSLKLSSV | |
| | | | | | | | | | TAADTAVFYCARMSRGYNYAYTFDIWG | |
| | | | | | | | | | QGTMVTVSS | |
| CoV2- | Ab | SARS-CoV2 | SARS- | SARS-CoV2 | | S; RBD | B-cells; SARS-CoV2 | 616 | QVTLRESGPALVKPTQTLTLTCTFSGFSLS | 1695 |
| 2919 | | | CoV1 | | | | Human Patient | | TSGMCLSWIRQPPGKALEWLARIDWD | |
| | | | | | | | | | DDKYYSTSLETRLTISKDTSKNQVVLTMT | |
| | | | | | | | | | NMDPVDTGTYYCARTMATINAFDIWG | |
| | | | | | | | | | QGTMVTVSS | |
| CoV2- | Ab | SARS-CoV1, | | | SARS-CoV2 | S; non- | B-cells; SARS-CoV2 | 617 | QVQLVESGGGVVQPGRSLRLSCAASGF | 1696 |
| 2933 | | SARS-CoV2 | | | | RBD | Human Patient | | TFSYYPMHWLWVRQAPGKGLEWVAVT | |
| | | | | | | | | | SYDGTNKYYADSVKGRFTISRDNSKNTLY | |
| | | | | | | | | | LQMNSLRAEDTAVYYCARGGATNFDY | |
| | | | | | | | | | WGQGTLVTVSS | |
| CoV2- | Ab | SARS-CoV2 | SARS- | | SARS-CoV1, | S; non- | B-cells; SARS-CoV2 | 618 | QVQLVQSGSELKKPGASVKVSCKASEYT | 1697 |
| 2934 | | | CoV1 | | SARS-CoV2 | RBD | Human Patient | | FTSYAMNWVRQAPGQGLEWMGWINT | |
| | | | | | | | | | NTGNPTYAQGFTGRFVFSLDTSVSTAYL | |
| | | | | | | | | | QISSLKAEDTAVYYCARPGKAAAFDYWG | |
| | | | | | | | | | QGTLVTVSS | |
| CoV2- | Ab | SARS-CoV2 | SARS- | | SARS-CoV1, | S; RBD | B-cells; SARS-CoV2 | 619 | QVQLVESGGGVVQPGRSLRLSCAASGF | 1698 |
| 2939 | | | CoV1 | | SARS-CoV2 | | Human Patient | | TFSSYGMHWVRQAPGKGLEWVAVIWY | |
| | | | | | | | | | DGSNKYYAESVKGRFTISRDNSKNTLYLQ | |
| | | | | | | | | | MNSLRAEDTAVYYCARDLHQDWVVVV | |
| | | | | | | | | | AANVYGMDVWGQGTTVTVSS | |
| CoV2- | Ab | SARS-CoV2 | SARS- | SARS-CoV2 | | S; RBD | B-cells; SARS-CoV2 | 620 | QMQLVQSGPEVKKPGTSVKVSCKASGF | 1699 |
| 2941 | | | CoV1 | (weak) | | | Human Patient | | TFMSSAVQWVRQARGQRLEWIGWIVI | |
| | | | | | | | | | GSGNTNYAQKFQERVTITRDMSTSTAY | |
| | | | | | | | | | MELSSLRSEDTAVYYCAAPYCSSISCNDG | |
| | | | | | | | | | FDIWGQGTMVTVSS | |
| CoV2- | Ab | SARS-CoV2 | SARS- | | SARS-CoV1, | S; non- | B-cells; SARS-CoV2 | 621 | QVTLRESGPALVKPTQTLTLTCTFSGFSLS | 1700 |
| 2942 | | | CoV1 | | SARS-CoV2 | RBD | Human Patient | | TSGLCVSWIRQPPGKALEWLARIDWDD | |
| | | | | | | | | | DKYYNTSLRTRLTISKDTSKNQVVLTMTN | |
| | | | | | | | | | MDPVDTATYYCARATTFFYGMDVWGQ | |
| | | | | | | | | | GTTATVSS | |
| CoV2- | Ab | SARS-CoV1, | | | SARS-CoV2 | S; RBD | B-cells; SARS-CoV2 | 622 | QVQLVESGGGVVQPGRSLRLSCAASGF | 1701 |
| 2944 | | SARS-CoV2 | | | | | Human Patient | | TFRRYGMYWVRQAPGKGLEWVAVISY | |
| | | | | | | | | | DGTDKYYTDSVKGRFTISRDNSKNTLYLQ | |
| | | | | | | | | | MNSLRAEDTAVYYCAKKGGPYCGGGN | |
| | | | | | | | | | CYAGYFDYWGQGILVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | DIVMTQSPDSLAVSLGERATINCKSSQ | IGHV3-30 | IGHJ4 | IGKV4-1 | IGKJ1 | 2696 | AKDGSIAAAD | 3898 | QQYYSTP | Seth Zost et al., 2020 |
| 2883 | SVLHSSNNKDSLVWYQQKPGQPPKLL | (Human) | (Human) | (Human) | (Human) | | Y | | WT | (https://www.nature.co |
| | IYWASSRESGVPDRFSGSGSGTDFTLTI | | | | | | | | | m/articles/s41591-020- |
| | SSLQAEDVAVYYCQQYYSTPWTFGQG | | | | | | | | | 0998-x) |
| | TKVEIK | | | | | | | | | |
| CoV2- | DIQMTQSPSTLSASVGDRVTITCRASQ | IGHV1-46 | IGHJ4 | IGKV1-5 | IGKJ2 | 2697 | ARGAAVPAA | 3899 | QQYNSYSY | Seth Zost et al., 2020 |
| 2891 | SISSWLAWYQQKPGKAPKLLIYKASSL | (Human) | (Human) | (Human) | (Human) | | GEFDY | | T | (https://www.nature.co |
| | ESGVPSRFSGSGSGTEFTLTISSLQPDD | | | | | | | | | m/articles/s41591-020- |
| | FATYYCQQYNSYSYTFGQGTKLEIK | | | | | | | | | 0998-x) |
| CoV2- | EIVMTQSPATLSVSPGERATLSCRASQ | IGHV3-30 | IGHJ4 | IGKV3-15 | IGKJ4 | 2698 | AKGDGSYLM | 3900 | QQYDDWP | Seth Zost et al., 2020 |
| 2894 | SVSNNLAWYQQKPGQAPRLLIYGAST | (Human) | (Human) | (Human) | (Human) | | DYFDY | | PEVT | (https://www.nature.co |
| | RATGIPVRFSGSGSGTEFTLTISSLQSE | | | | | | | | | m/articles/s41591-020- |
| | DFAVYYCQQYDDWPPEVTFGG | | | | | | | | | 0998-x) |
| | GTKVEIK | | | | | | | | | |
| CoV2- | QSVLTQPPSVSGAPGQRVTISCTGSSS | IGHV3-53 | IGHJ3 | IGLV1-40 | IGLJ2 | 2699 | ARSYDILTGYR | 3901 | VAWDDSR | Seth Zost et al., 2020 |
| 2901 | NIGSGSDVHWYQQLPGTAPKLLIYGN | (Human) | (Human) | (Human) | (Human) | | DAFDI | | NGLV | (https://www.nature.co |
| | TNRPSGVPDRFSGSKSGTSASLAITGL | | | | | | | | | m/articles/s41591-020- |
| | QAEDEADYYCVAWDDSRNGLVFGGG | | | | | | | | | 0998-x) |
| | AKLTVL | | | | | | | | | |
| CoV2- | QSVLTQPPSVSGVPGQRVTVSCTGSSS | IGHV4-34 | IGHJ6 | IGLV1-40 | IGLJ2 | 2700 | ARCRQMGNF | 3902 | QSFDIGRG | Seth Zost et al., 2020 |
| 2904 | NIGAGFDVYWYQQFLGTAPKLLIYGN | (Human) | (Human) | (Human) | (Human) | | YYYYMDV | | GWI | (https://www.nature.co |
| | NNRPSGVPDRFSASKSGTSASLAITGL | | | | | | | | | m/articles/s41591-020- |
| | QAEDEADYYCQSFDIGRGGWIFGGGT | | | | | | | | | 0998-x) |
| | KLTVL | | | | | | | | | |
| CoV2- | DIQMTQSPSSLSASVGDRVTITCRPSQ | IGHV5-51 | IGHJ6 | IGKV1-39 | IGKJ5 | 2701 | ARLGSESKIDY | 3903 | QQSYSTPP | Seth Zost et al., 2020 |
| 2906 | SITTYLNWYQQKPGKAPRLLIYAVSSLQ | (Human) | (Human) | (Human) | (Human) | | YYYGMDV | | T | (https://www.nature.co |
| | SGVPSRFSGSGSGTDFTLTISSLQPEDF | | | | | | | | | m/articles/s41591-020- |
| | ATYYCQQSYSTPPTFGQGTRLEIK | | | | | | | | | 0998-x) |
| CoV2- | QSVLTQPPSVSGAPGQRVTISCTGSSS | IGHV1-69 | IGHJ6 | IGLV1-40 | IGLJ2 | 2702 | ARGRGYSNY | 3904 | QSYDSSLS | Seth Zost et al., 2020 |
| 2909 | NIGAGYDVHWYQQLPETAPKLLIYAN | (Human) | (Human) | (Human) | (Human) | | GASYYMDV | | GSV | (https://www.nature.co |
| | SNRPSGVPDRFSGSKSGTSASLAITGL | | | | | | | | | m/articles/s41591-020- |
| | QAEDEADYYCQSYDSSLSGSVFGGGT | | | | | | | | | 0998-x) |
| | KLTVL | | | | | | | | | |
| CoV2- | QSALTQPASVSGSPGQSITISCTGTSSD | IGHV4-39 | IGHJ3 | IGLV2-23 | IGLJ2 | 2703 | ARMSRGYNY | 3905 | CSYAGSVL | Seth Zost et al., 2020 |
| 2911 | VGSYNLVSWYQQHPGKAPKLKIYEGS | (Human) | (Human) | (Human) | (Human) | | AYTFDI | | | (https://www.nature.co |
| | KRPSGVSNRFSGSKSGNTASLTISGLQ | | | | | | | | | m/articles/s41591-020- |
| | AEDEADYYCCSYAGSVLFGGGTKLTVL | | | | | | | | | 0998-x) |
| CoV2- | DIQMTQSPSSLSASAGDRVTITCRASQ | IGHV2-70 | IGHJ3 | IGKV1-39 | IGKJ2 | 2704 | ARTMATINAF | 3906 | QQSFSTPR | Seth Zost et al., 2020 |
| 2919 | SIRYYLNWYQQKPGKAPKLLIYAASSL | (Human) | (Human) | (Human) | (Human) | | DI | | T | (https://www.nature.co |
| | QSGVPSRFSGSGSGTDFTLTISSLQPED | | | | | | | | | m/articles/s41591-020- |
| | FAAYYCQQSFSTPRTFGQGTKLEIK | | | | | | | | | 0998-x) |
| CoV2- | SYVLTQPPSVSVAPGKTANITCGGNNI | IGHV3-30- | IGHJ4 | IGLV3-21 | IGLJ3 | 2705 | ARGGATNFD | 3907 | QVWDSSS | Seth Zost et al., 2020 |
| 2933 | GRKSVHWYQQKSGQAPVLVVYDDSD | 3 | (Human) | (Human) | (Human) | | Y | | DHPEWV | (https://www.nature.co |
| | RPSGIPERFSGSNSGNTATLTISRVEAG | (Human) | | | | | | | | m/articles/s41591-020- |
| | DEADYYCQVWDSSSDHPEWVFGGGT | | | | | | | | | 0998-x) |
| | KLTVL | | | | | | | | | |
| CoV2- | DIVMTQSPLSLPVTPGEPASISCRSSQS | IGHV7-4- | IGHJ4 | IGKV2-28 | IGKJ1 | 2706 | ARPGKAAAF | 3908 | MQALQTP | Seth Zost et al., 2020 |
| 2934 | LLHSNGYNFLDWYLQKPGQSPQLLIYL | 1 | (Human) | (Human) | (Human) | | DY | | WT | (https://www.nature.co |
| | GSNRASGVPDRFSGSGSGTDFTLKISR | (Human) | | | | | | | | m/articles/s41591-020- |
| | VQAEDVGVYYCMQALQTPWTFGQG | | | | | | | | | 0998-x) |
| | TKVEIK | | | | | | | | | |
| CoV2- | SSELTQDPAVSVALGQTVRITCQGDSL | IGHV3-33 | IGHJ6 | IGLV3-19 | IGLJ3 | 2707 | ARDLHQDWV | 3909 | NSRDSSGN | Seth Zost et al., 2020 |
| 2939 | RRYYASWYQQKPGQAPVLVIYGKNN | (Human) | (Human) | (Human) | (Human) | | VVVAANVYG | | PRW | (https://www.nature.co |
| | RPSGIPDRFSGSSSGNTASLTITGAQAE | | | | | | MDV | | | m/articles/s41591-020- |
| | DEADYYCNSRDSSGNPRWFGGGTKLT | | | | | | | | | 0998-x) |
| | VL | | | | | | | | | |
| CoV2- | EIVLTQSPGTLSLSPGERATLSCRASQS | IGHV1-58 | IGHJ3 | IGKV3-20 | IGKJ2 | 2708 | AAPYCSSISCN | 3910 | QQYGSSPP | Seth Zost et al., 2020 |
| 2941 | VSSSYLAWYQQKPGQAPRLLIYGASSR | (Human) | (Human) | (Human) | (Human) | | DGFDI | | RYT | (https://www.nature.co |
| | ATGIPDRFSGSGSGTDFTLTISRLEPED | | | | | | | | | m/articles/s41591-020- |
| | FAVYYCQQYGSSPPRYTFGQGTKLEIK | | | | | | | | | 0998-x) |
| CoV2- | SYELTQPPSVSVSPGQTARITCSTDALP | IGHV2-70 | IGHJ6 | IGLV3-25 | IGLJ3 | 2709 | ARATTFFYGM | 3911 | QSVDSSGT | Seth Zost et al., 2020 |
| 2942 | NEYIYWYQQKPGQAPVLVIYKDSERPS | (Human) | (Human) | (Human) | (Human) | | DV | | YRV | (https://www.nature.co |
| | GIPERFSGSSSGTTVTLTISGVQAEDEA | | | | | | | | | m/articles/s41591-020- |
| | DYYCQSVDSSGTYRVFGGGTKLTVL | | | | | | | | | 0998-x) |
| CoV2- | DIQMTQSPSSLSASVGDRVTITCQASQ | IGHV3-30 | IGHJ4 | IGKV1-33 | IGKJ3 | 2710 | AKKGGPYCG | 3912 | QQYDNLPL | Seth Zost et al., 2020 |
| 2944 | DISNYLNWYQQKAGKAPKLLIYDASNL | (Human) | (Human) | (Human) | (Human) | | GGNCYAGYF | | A | (https://www.nature.co |
| | ETGVPSRFSGSGSGTDFTFTISSLQPDD | | | | | | DY | | | m/articles/s41591-020- |
| | IATYYCQQYDNLPLAFGPGTKVDVK | | | | | | | | | 0998-x) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2- | Ab | SARS-CoV1, | | | SARS-CoV2 | S; RBD | B-cells; SARS-CoV2 | 623 | EVQVVESGGGLVQPGRSLRLSCAASGFT | 1702 |
| 2945 | | SARS-CoV2 | | | | | Human Patient | | FEDYAMHWVRQAPGKGLEWVSGVSW | |
| | | | | | | | | | NSGIIGYADSVKGRFTISRDNAKNSLYLQ | |
| | | | | | | | | | MNSLRAEDTALYYCAKLDVGGYDFVSG | |
| | | | | | | | | | HYYAFDIWGQGTMVTVSS | |
| CoV2- | Ab | SARS-CoV2 | SARS- | | SARS-CoV1, | S; non- | B-cells; SARS-CoV2 | 624 | QVQLVQSGSELKKPGASVKVSCKASGYT | 1703 |
| 2947 | | | CoV1 | | SARS-CoV2 | RBD | Human Patient | | FSDYAMNWVRQAPGQGLEWMGWIN | |
| | | | | | | | | | TNTGNPTYAQGFTGRFVFSLDTSVSTAY | |
| | | | | | | | | | LQISSLKAEDTAVYYCARGLISLFRGAIFH | |
| | | | | | | | | | YYYGMDVWGQGTTVTVSS | |
| CoV2- | Ab | SARS-CoV2 | SARS- | SARS-CoV2 | | S; RBD | B-cells; SARS-CoV2 | 625 | EVQLVESGGGLVQPGGSLRLSCAASGLT | 1704 |
| 2952 | | | CoV1 | | | | Human Patient | | VRSNYMTWVRQTPGKGLEWVSVIYSG | |
| | | | | | | | | | GSTFYADSVKGRFTISRDNSKNTVYLQM | |
| | | | | | | | | | NSLRAEDTAVYYCARDLVTYGLDVWGQ | |
| | | | | | | | | | GTTVTVSS | |
| CoV2- | Ab | SARS-CoV1, | | | SARS-CoV2 | S; RBD | B-cells; SARS-CoV2 | 626 | QITLKESGPTLVKPTQTLTLTCTFSGFSLS | 1705 |
| 2953 | | SARS-CoV2 | | | | | Human Patient | | TKRVGVGWIRQPPGKALEWLALIYWDD | |
| | | | | | | | | | DQRYSPSLKSRLTITKDTSKNQVVLTMT | |
| | | | | | | | | | NMDPVDTATYYCAHSGPPDLSPVLSQG | |
| | | | | | | | | | WFDPWGQGTLVTVSS | |
| CoV2- | Ab | SARS-CoV2 | SARS- | SARS-CoV2 | | S; RBD | B-cells; SARS-CoV2 | 627 | QVQLVESGGGVVQPGRSLRLSCAASGF | 1706 |
| 2955 | | | CoV1 | | | | Human Patient | | TFVTSGIHWVRQAPGKGLEWVAVISYD | |
| | | | | | | | | | GSNKYYADSVKGRFTISRDNSKNTLYLQ | |
| | | | | | | | | | MNSLRAEDTAVYYCAKGGPNKEVLYFG | |
| | | | | | | | | | ELLDYGMDVWGQGTTVTVSS | |
| CoV2- | Ab | SARS-CoV1, | | | SARS-CoV2 | S; non- | B-cells; SARS-CoV2 | 628 | QVQVVQSGAEVKKPGASVKVSCKASGY | 1707 |
| 2960 | | SARS-CoV2 | | | | RBD | Human Patient | | TFKNYGISWVRQAPGQGLEWMGWISA | |
| | | | | | | | | | YTGNTNYAQKFQGRMTMTTDTSTGTG | |
| | | | | | | | | | YMELRSLRSDDTAVYYCARVQRRRLDY | |
| | | | | | | | | | WGQGTLVIVSS | |
| CoV2- | Ab | SARS-CoV2 | SARS- | SARS-CoV2 | | S; RBD | B-cells; SARS-CoV2 | 629 | QMQLVQSGPEVKKPGTSVKVSCKASGF | 1708 |
| 2961 | | | CoV1 | | | | Human Patient | | TFMSSAVQWVRQARGQRLEWIGWIVI | |
| | | | | | | | | | GSGNTNYAQKFQERVTITRDMSTSTAY | |
| | | | | | | | | | MELSSLRSEDTAVYYCAAPYCSSISCNDG | |
| | | | | | | | | | FDIWGQGTMVTVSS | |
| CoV2- | Ab | SARS-CoV2 | SARS- | | SARS-CoV1, | S; RBD | B-cells; SARS-CoV2 | 630 | QVQLVQSGAEVKKPGASVKVSCKASGY | 1709 |
| 2997 | | | CoV1 | | SARS-CoV2 | | Human Patient | | TFTSYYMHWVRQAPGQGLEWMGIINP | |
| | | | | | | | | | GGGSTTYAQKFQGRVTMTSDTSTSTVY | |
| | | | | | | | | | MELSSLRSEDTAMYYCARGAIPPNSRAE | |
| | | | | | | | | | IDYWGQGTLVTVSS | |
| CoV2- | Ab | SARS-CoV2 | SARS- | | SARS-CoV1, | S; RBD | B-cells; SARS-CoV2 | 631 | QVQLVESGGGVVQPGRSLRLSCAASGF | 1710 |
| 3010 | | | CoV1 | | SARS-CoV2 | | Human Patient | | TFSDYAMHWVRQAPGKGLEWVADISF | |
| | | | | | | | | | DGSNKYYADSVKGRFTISRDSSENTLYLQ | |
| | | | | | | | | | MDSLRADDTAVYYCARDLSTTWYLEM | |
| | | | | | | | | | WGPDAFDIWGQGTVVTVSS | |
| CoV2- | Ab | SARS-CoV2 | SARS- | | SARS-CoV1, | S; RBD | B-cells; SARS-CoV2 | 632 | EVQLVESGGGLVQPGGSLRLSCAASGFT | 1711 |
| 3012 | | | CoV1 | | SARS-CoV2 | | Human Patient | | FSSYDLHWVRQGTGKRLEWVSAIGTAG | |
| | | | | | | | | | DTYYLGSVKGRFTISRENAKNSLYLQMN | |
| | | | | | | | | | SLRAGDTAVYYCARVLYDSSGFYNWFDP | |
| | | | | | | | | | WGQGTLVTVSS | |
| CoV2- | Ab | SARS-CoV2 | SARS- | | SARS-CoV1, | S; RBD | B-cells; SARS-CoV2 | 633 | QVQLVESGGGVVQPGRSLRLSCAASGF | 1712 |
| 3013 | | | CoV1 | | SARS-CoV2 | | Human Patient | | TFSNYGMHWVRQAPGKGLEWVAVMS | |
| | | | | | | | | | YDGSNKYYADSVKGRFTISRDNSKNTLYL | |
| | | | | | | | | | QMNSLRAEDTAVYYCAKNLGPYCSGGT | |
| | | | | | | | | | CYSLVGDYWGQGTLVTVSS | |
| CoV2- | Ab | SARS-CoV2 | SARS- | SARS-CoV2 | | S; RBD | B-cells; SARS-CoV2 | 634 | EVQLVQSGPEVKKPGTSVKVSCKASGFT | 1713 |
| 3025 | | | CoV1 | | | | Human Patient | | FMSSAVQWVRQARGQRLEWIGWIVIG | |
| | | | | | | | | | SGNTNYAQKFQERVTITRDMSTSTAYM | |
| | | | | | | | | | ELSSLRSEDTAVYYCAAPYCSSISCNDGF | |
| | | | | | | | | | DIWGQGTMVTVSS | |
| CoV2- | Ab | SARS-CoV1, | | | SARS-CoV2 | S; RBD | B-cells; SARS-CoV2 | 635 | EVQLVESGGGVVQPGGSLRLSCAASGF | 1714 |
| 3029 | | SARS-CoV2 | | | | | Human Patient | | TEDDYAMHWVRQAPGKGLEWVSLISG | |
| | | | | | | | | | DGGNTYYADSVKGRFTISRDNSKNSLYL | |
| | | | | | | | | | QMNSLRTEDTALYYCAKDEMAYPPSHH | |
| | | | | | | | | | YYYYYMDVWGKGTTVTVSS | |
| CoV2- | Ab | SARS-CoV1, | | | SARS-CoV2 | S; non- | B-cells; SARS-CoV2 | 636 | EVQLVESGGGLVQPGGSLRLSCAASGFT | 1715 |
| 3057 | | SARS-CoV2 | | | | RBD | Human Patient | | FSSYWMSWVRQAPGKGLEWVANIKQ | |
| | | | | | | | | | DGSEKYFVDSVKGRFTISRDNAKNSLYL | |
| | | | | | | | | | QMNSLRAEDAAVYYCARLVTTVTTANG | |
| | | | | | | | | | LYYYSYYYMDVWGKGTTVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2- | DVVMTQSPLSLPVTLGQPASISCRSSQ | IGHV3-9 | IGHJ3 | IGKV2-30 | IGKJ2 | 2711 | AKLDVGGYDF | 3913 | MQGTHW | Seth Zost et al., 2020 |
| 2945 | SLVYSDGNTYLNWFQQRPGQSPRRLI | (Human) | (Human) | (Human) | (Human) | | VSGHYYAFDI | | PYT | (https://www.nature.co |
| | YKVSSRDSGVPDRFSGSGSGTAFTLKIS | | | | | | | | | m/articles/s41591-020- |
| | RVEAEDVGVYYCMQGTHWPYTFGQ | | | | | | | | | 0998-x) |
| | GTKLEIK | | | | | | | | | |
| CoV2- | QSALTQPRSVSGSPGQSVTISCTGTSS | IGHV7-4- | IGHJ6 | IGLV2-11 | IGLJ3 | 2712 | ARGLISLFRGA | 3914 | CSYAGRYT | Seth Zost et al., 2020 |
| 2947 | DVGGYTYVSWYQQHPGKAPKLMIYD | (Human) | (Human) | (Human) | (Human) | | IFHYYYGMDV | | WV | (https://www.nature.co |
| | VNKRPSGVPDRFSGSKSGITASLTISGL | | | | | | | | | m/articles/s41591-020- |
| | QAEDEADYYCCSYAGRYTWVFGGGTT | | | | | | | | | 0998-x) |
| | LTVL | | | | | | | | | |
| CoV2- | DIQLTQSPSFLSASVGDRVTITCRASQG | IGHV3-66 | IGHJ6 | IGKV1-9 | IGKJ5 | 2713 | ARDLVTYGLD | 3915 | QLLNSHPL | Seth Zost et al., 2020 |
| 2952 | ISNYLAWYQQKPGTAPNLLIYAASTLQ | (Human) | (Human) | (Human) | (Human) | | V | | T | (https://www.nature.co |
| | SGVPSRFSGSGSGTEFTLTISSLQPEDF | | | | | | | | | m/articles/s41591-020- |
| | ATYYCQLLNSHPLTFGQGTRLEIK | | | | | | | | | 0998-x) |
| CoV2- | SYELTQPPSVSVSPGQTARITCSGDALP | IGHV2-5 | IGHJ5 | IGLV3-25 | IGLJ3 | 2714 | AHSGPPDLSP | 3916 | QSADSTG | Seth Zost et al., 2020 |
| 2953 | KQYAYWYQQKPGQAPVLVIYRDSERP | (Human) | (Human) | (Human) | (Human) | | VLSQGWFDP | | WV | (https://www.nature.co |
| | SGIPERFSGSTSGTTVTLTISGVQAEDE | | | | | | | | | m/articles/s41591-020- |
| | ADYYCQSADSTGWVFGGGTKLTVL | | | | | | | | | 0998-x) |
| CoV2- | DIVMTQTPLSLSVTPGQPASFSCKSSQ | IGHV3-30 | IGHJ6 | IGKV2D-29 | IGKJ2 | 2715 | AKGGPNKEVL | 3917 | MQSIQLPY | Seth Zost et al., 2020 |
| 2955 | SLLHSDGKTYLYWYLQKPGQSPQLLIY | (Human) | (Human) | (Human) | (Human) | | YFGELLDYGM | | T | (https://www.nature.co |
| | AVSNRFSGVPDRFSGSGSGTDFTLKIS | | | | | | DV | | | m/articles/s41591-020- |
| | RVEAEDVGVYYCMQSIQLPYTFGQGT | | | | | | | | | 0998-x) |
| | KLEIK | | | | | | | | | |
| CoV2- | DIVVTQTPLSLSVTPGQPASISCKSSETL | IGHV1-18 | IGHJ4 | IGKV2D-29 | IGKJ5 | 2716 | ARVQRRRLDY | 3918 | MQSIQLA | Seth Zost et al., 2020 |
| 2960 | LHSDGKTYLSWYLQKPGQPPQLLIYEV | (Human) | (Human) | (Human) | (Human) | | | | | (https://www.nature.co |
| | SNRFSGVPDRFSGSGSGTDFTLKIGRV | | | | | | | | | m/articles/s41591-020- |
| | EAEDVGLYYCMQSIQLAFGQGTRLEIE | | | | | | | | | 0998-x) |
| CoV2- | ETVLTQSPGTLSLSPGERATLSCRASQS | IGHV1-58 | IGHJ3 | IGKV3-20 | IGKJ1 | 2717 | AAPYCSSISCN | 3919 | QHYGSSRG | Seth Zost et al., 2020 |
| 2961 | VSSSYLAWYQQKPGQAPRLLIYGASSR | (Human) | (Human) | (Human) | (Human) | | DGFDI | | WT | (https://www.nature.co |
| | ATGIPDRFSGSGSGTDFTLTISRLEPED | | | | | | | | | m/articles/s41591-020- |
| | FAVYYCQHYGSSRGWTFGQGTKVEIK | | | | | | | | | 0998-x) |
| CoV2- | EIVMTQSPATLSVSPGERVTLSCRASQ | IGHV1-46 | IGHJ4 | IGKV3-15 | IGKJ4 | 2718 | ARGAIPPNSR | 3920 | QQYYNWP | Seth Zost et al., 2020 |
| 2997 | SVSSNLAWCQQKPGQAPRLLIYGAST | (Human) | (Human) | (Human) | (Human) | | AEIDY | | LT | (https://www.nature.co |
| | RATGIPARFSGSGSGTEFTLTISSLQSE | | | | | | | | | m/articles/s41591-020- |
| | DFAVYYCQQYYNWPLTFGGGTKVEIK | | | | | | | | | 0998-x) |
| CoV2- | DIQMTQSPSTLSASIGDRVTITCRASQS | IGHV3-30- | IGHJ3 | IGKV1-5 | IGKJ2 | 2719 | ARDLSTTWYL | 3921 | QQYNSYPY | Seth Zost et al., 2020 |
| 3010 | ISSWLAWYQQIPGKAPKLLIYKASSLES | 3 | (Human) | (Human) | (Human) | | EMWGPDAF | | T | (https://www.nature.co |
| | GVPSRFSGSGSGTEFTLTISSLQPDDFA | (Human) | | | | | DI | | | m/articles/s41591-020- |
| | TYYCQQYNSYPYTFGQGTKLEIK | | | | | | | | | 0998-x) |
| CoV2- | DIQMTQSPSSLSASVGDRVTITCRASQ | IGHV3-13 | IGHJ5 | IGKV1-39 | IGKJ1 | 2720 | ARVLYDSSGF | 3922 | QQSYEIPP | Seth Zost et al., 2020 |
| 3012 | SISSYLNWYQQKPGKAPKLLIYAASSLE | (Human) | (Human) | (Human) | (Human) | | YNWFDP | | WT | (https://www.nature.co |
| | SGVPSRFSGSGSGTDFTLTISSLQPEDF | | | | | | | | | m/articles/s41591-020- |
| | ATYYCQQSYEIPPWTFGQGTKVEIK | | | | | | | | | 0998-x) |
| CoV2- | DIQMTQSPSSLSASVGDRVTITCQASQ | IGHV3-30 | IGHJ4 | IGKV1-33 | IGKJ3 | 2721 | AKNLGPYCSG | 3923 | QQYANLPF | Seth Zost et al., 2020 |
| 3013 | DISNYLNWYQQKPGKAPKLLIYDASNL | (Human) | (Human) | (Human) | (Human) | | GTCYSLVGDY | | T | (https://www.nature.co |
| | ETGVPSRFSGSGSGTDFTFTISSLQPED | | | | | | | | | m/articles/s41591-020- |
| | IATYYCQQYANLPFTFGPGTKVDIK | | | | | | | | | 0998-x) |
| CoV2- | EIVLTQSPGTLSLSPGERATLSCRASQS | IGHV1-58 | IGHJ3 | IGKV3-20 | IGKJ1 | 2722 | AAPYCSSISCN | 3924 | QHYGSSRG | Seth Zost et al., 2020 |
| 3025 | VSSSYLAWYQQKPGQAPRLLIYGASSR | (Human) | (Human) | (Human) | (Human) | | DGFDI | | WT | (https://www.nature.co |
| | ATGIPDRFSGSGSGTDFTLTISRLEPED | | | | | | | | | m/articles/s41591-020- |
| | FAVYYCQHYGSSRGWTFGQGTKVEIK | | | | | | | | | 0998-x) |
| CoV2- | SYELTQPPSVSVSPGQTARITCSTDALP | IGHV3-43 | IGHJ6 | IGLV3-25 | IGLJ2 | 2723 | AKDEMAYPP | 3925 | QSVDSSGT | Seth Zost et al., 2020 |
| 3029 | NEYIYWYQQKPGQAPVLVIYKDSERPS | (Human) | (Human) | (Human) | (Human) | | SHHYYYYYMD | | YPHVI | (https://www.nature.co |
| | GIPERFSGSSSGTTVTLTISGVQAEDEA | | | | | | V | | | m/articles/s41591-020- |
| | DYYCQSVDSSGTYPHVIFGGGTKLTVL | | | | | | | | | 0998-x) |
| CoV2- | QSVLTQPPSVSGAPGQRVTISCTGSSS | IGHV3-7 | IGHJ6 | IGLV1-40 | IGLJ2 | 2724 | ARLVTTVTTA | 3926 | QSYASSLS | Seth Zost et al., 2020 |
| 3057 | NIGAGYDVHWYQQLPGTAPKLLIYGN | (Human) | (Human) | (Human) | (Human) | | NGLYYYSYYY | | AHVV | (https://www.nature.co |
| | TNRPSGVPDRFSGSKSGTSASLAITGL | | | | | | | | | m/articles/s41591-020- |
| | QAEDEADYYCQSYASSLSAHVVFGGG | | | | | | | | | 0998-x) |
| | TKLTVL | | | | | | | | | |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoV2 | Ab | SARS-CoV1, | | | SARS-CoV2 | S; non- | B-cells; SARS-CoV2 | 637 | EVQLVESGGGAVQPGRSLRLSCAASGFT | 1716 |
| 3058 | | SARS-CoV2 | | | | RBD | Human Patient | | FSTYAMYWVRQAPGKGLEWVAVISYD | |
| | | | | | | | | | GSNRYYADSVKGRFTISRDNSKNTLYLQ | |
| | | | | | | | | | MNSLRPEDTAVYYCARDRSGNYRDAFD | |
| | | | | | | | | | IWGQGTMVTVSS | |
| CoVA1- | Ab | SARS-CoV2 | SARS- | | SARS-CoV1, | S; non- | B-cells; SARS-CoV2 | 638 | HQHQVEQGPGGVEQSETLFLTCCVSGG | 1717 |
| 01 | | | CoV1 | | SARS-CoV2 | RBD | Human Patient | | FTISSCYKRGGIRQPPRKGEEWAVSSYYS | |
| | | | | | | | | | SSTYYTPSLKSRVTISVDKSKNQFSLKMSS | |
| | | | | | | | | | VTAADTAVYYCARVSSGYYFTPFDYWG | |
| | | | | | | | | | QGTRGHRLF | |
| CoVA1- | Ab | SARS-CoV2 | SARS- | | SARS-CoV1, | S; non- | B-cells; SARS-CoV2 | 639 | EVQLVESGGGVVQPGRSLRLSCAASGFT | 1718 |
| 02 | | | CoV1 | | SARS-CoV2 | RBD | Human Patient | | FSIYAMHWVRQAPGKGLEWVAVISYD | |
| | | | | | | | | | GSNQYYADSVRGRFTISRGNSKNTLYLQ | |
| | | | | | | | | | MNSLRPEDTAVYYCARARGGSYNDAFD | |
| | | | | | | | | | IWGQGTMVTVSS | |
| CoVA1- | Ab | SARS-CoV2 | SARS- | SARS-CoV2 | SARS-CoV1 | Unk | B-cells; SARS-CoV2 | 640 | EVQLVESGGGVVQPGRSLRLSCAASGFT | 1719 |
| 03 | | | CoV1 | | | | Human Patient | | FSSYGMHWVRQAPGKGLEWVAVISYD | |
| | | | | | | | | | GSNKYYADSVKGRFTISRDNSKNTLYLQ | |
| | | | | | | | | | MNSLRAEDTAVYYCAKDGQYYDFWSGY | |
| | | | | | | | | | LGARTNPHYYYYMDVWGKGTMVTVSS | |
| CoVA1- | Ab | SARS-CoV2 | SARS- | | SARS-CoV1, | S; non- | B-cells; SARS-CoV2 | 641 | EVQLVQSGGGLVQPGRSLRLSCAASGFT | 1720 |
| 06 | | | CoV1 | | SARS-CoV2 | RBD | Human Patient | | FGEYAMHWVRQAPGKGLEWVSGISW | |
| | | | | | | | | | NSGSIDYADSVKGRFTISRDNAKNSLYLQ | |
| | | | | | | | | | MNSLRAEDTALYYCAKDMGEAVAGTHY | |
| | | | | | | | | | GMDVWGQGTMVTASS | |
| CoVA1- | Ab | SARS-CoV1, | | | SARS-CoV1, | S; non- | B-cells; SARS-CoV2 | 642 | QVQLVESGAEVKKPGSSVKVSCKASGGT | 1721 |
| 07 | | SARS-CoV2 | | | SARS-CoV2 | RBD | Human Patient | | LSSYAITWVRQAPGQGLEWVGGIIPIFG | |
| | | | | | | | | | TANYAQKFQGRVTITADESTSTAYMELS | |
| | | | | | | | | | SLRSEDTAVYYCARVGAYDSSGYSNDYW | |
| | | | | | | | | | GQGTLVTVSS | |
| CoVA1- | Ab | SARS-CoV2 | SARS- | | SARS-CoV1, | S; RBD | B-cells; SARS-CoV2 | 643 | EVQLVESGGGVVQPGGSLRLSCAASGF | 1722 |
| 08 | | | CoV1 | | SARS-CoV2 | | Human Patient | | TFSSYAMHWVRQAPGKGLEWVAVISY | |
| | | | | | | | | | DGSNKYYADSVKGRFTISRDNSKNTLYL | |
| | | | | | | | | | QMNSLRAEDTAVYYCAREDYYDSSGSF | |
| | | | | | | | | | DYWGQGTLVTVSS | |
| CoVA1- | Ab | SARS-CoV2 | SARS- | | SARS-CoV1, | S; non- | B-cells; SARS-CoV2 | 644 | QLQLVESGPGLVKPSETLSLTCTVSGGSI | 1723 |
| 09 | | | CoV1 | | SARS-CoV2 | RBD | Human Patient | | SSYFWSWIRQPPGKGLEWIGYIYYSGST | |
| | | | | | | | | | NYNPSLKSRVTISVDTSKNQFSLKLSSVTA | |
| | | | | | | | | | ADTAVYYCARHSQGWLQQAVAFDIWG | |
| | | | | | | | | | QGTMVTVSS | |
| CoVA1- | Ab | SARS-CoV2 | SARS- | | SARS-CoV1, | S; RBD | B-cells; SARS-CoV2 | 645 | EVQLVESGGGLVQPGGSLRLSCAASGFT | 1724 |
| 10 | | | CoV1 | | SARS-CoV2 | | Human Patient | | VTSTYMTWVRRAPGKGLEWVSIIYNDD | |
| | | | | | | | | | TTYYADSVKDRVTVSRDDSKNTLYLQM | |
| | | | | | | | | | NSLRAEDTAIYYCARGGYYYDPSGYYSRS | |
| | | | | | | | | | FSFDYWGQGTLVTVSS | |
| CoVA1- | Ab | SARS-CoV2 | SARS- | SARS-CoV2 | SARS-CoV1 | S; RBD | B-cells; SARS-CoV2 | 646 | EVQLVQSGTEVKKPGASVKVSCKASGYT | 1725 |
| 12 | | | CoV1 | | | | Human Patient | | FPGYYIHWVRQAPGQGLEWMGWINP | |
| | | | | | | | | | NSGVAKSAQKFQGRVTMTRDSSISTVYL | |
| | | | | | | | | | DVTSDDTAVYYCARDLVWATVSGTMD | |
| | | | | | | | | | VWGQGTTVTVSS | |
| CoVA1- | Ab | SARS-CoV1, | | SARS-CoV1, | | S; RBD | B-cells; SARS-CoV2 | 647 | QVQLVQSGAEVKKPGASVKVSCKASGY | 1726 |
| 16 | | SARS-CoV2 | | SARS-CoV2 | | | Human Patient | | TFTSYYMHWVRQAPGQGLEWMGIINS | |
| | | | | | | | | | SGGSTSYAQKFQGRVTMTRDTSTSTVY | |
| | | | | | | | | | MELSSLRSEDTAVYYCARPPRNYYDRSG | |
| | | | | | | | | | YYQRAEYFQHWGQGTLVTVSS | |
| CoVA1- | Ab | SARS-CoV2 | SARS- | SARS-CoV2 | SARS-CoV1 | S; RBD | B-cells; SARS-CoV2 | 648 | EVQLVESGGGLVQPGGSLRLSCAASGFT | 1727 |
| 18 | | | CoV1 | | | | Human Patient | | VSSNYMSWVRQAPGKGLEWVSVIYSG | |
| | | | | | | | | | GSTYYADSVKGRFTISRDNSKNTLYLQM | |
| | | | | | | | | | NSLRAEDTAVYYCARVEWAAAGTFYWG | |
| | | | | | | | | | QGTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoV2 | DIVMTQSPDSLAVSLGERATINCKSSQ | IGHV3-30 | IGHJ3 | IGKV4-1 | IGKJ2 | 2725 | ARDRSGNYR | 3927 | QQYYSSYT | Seth Zost et al., 2020 |
| 3058 | SVLYSSNNENYLAWYQQKPGQPPKLLI | (Human) | (Human) | (Human) | (Human) | | DAFDI | | | (https://www.nature.co |
| | YWASTRESGVPDRFSGSGSGTDFTLTI | | | | | | | | | m/articles/s41591-020- |
| | SSLQAEDVAVYYCQQYYSSYTFGQGTK | | | | | | | | | 0998-x) |
| | VEIK | | | | | | | | | |
| CoVA1- | EIVMTQSPGTLSLSPGERATLSCRASQ | IGHV4-39 | IGHJ4 | IGKV3-20 | IGKJ3 | 2726 | ARVSSGYYFT | 3928 | QQYGSSPP | Philip Brouwer et al., |
| 01 | SVSSSYLAWYQQKPGQAPRLLIYGASS | (Human) | (Human) | (Human) | (Human) | | PFDY | | PFT | 2020 |
| | RATGIPDRFSGSGSGTDFTLTISRLEPE | | | | | | | | | (https://science.science |
| | DFAVYYCQQYGSSPPPFTFGPGTKVDI | | | | | | | | | mag.org/content/early/ |
| | K | | | | | | | | | 2020/06/15/science.abc |
| | | | | | | | | | | 5902) |
| CoVA1- | NFMLTQPASVSGSPGQSITISCTGASS | IGHV3-30 | IGHJ3 | IGLV2-14 | IGLJ3 | 2727 | ARARGGSYN | 3929 | SSYTSSSTP | Philip Brouwer et al., |
| 02 | DVGGYNYVSWYQQHPGKAPKLMIYD | (Human) | (Human) | (Human) | (Human) | | DAFDI | | VV | 2020 |
| | VSNRPSGVSNRFSGSKSGNTASLTISGL | | | | | | | | | (https://science.science |
| | QAEDEADYYCSSYTSSSTPVVFGGGTE | | | | | | | | | mag.org/content/early/ |
| | LTVL | | | | | | | | | 2020/06/15/science.abc |
| | | | | | | | | | | 5902) |
| CoVA1- | DIVMTQSPSSLSASVGDRVTITCRASQ | IGHV3-30 | IGHJ6 | IGKV1-27 | IGKJ3 | 2728 | AKDGQYYDF | 3930 | QKYNSAPP | 2020/06/15/science.abc |
| 03 | GISNYLAWYQQKPGKVPKLLIYAASTL | (Human) | (Human) | (Human) | (Human) | | WSGYLGART | | A | Philip Brouwer et al., |
| | QSGVPSRFSGSGSGTDFTLTISSLQPED | | | | | | NPHYYYYMD | | | 2020 |
| | VATYYCQKYNSAPPAFGQGTKVDIK | | | | | | V | | | (https://science.science |
| | | | | | | | | | | mag.org/content/early/ |
| | | | | | | | | | | 5902) |
| CoVA1- | QSALTQPPSVSVSPGQTASIPCSGDKL | IGHV3-9 | IGHJ6 | IGLV3-1 | IGLJ3 | 2729 | AKDMGEAVA | 3931 | QAWGSTT | 2020/06/15/science.abc |
| 06 | GDIYACWYQQKPGQSPVLVIYQDTKR | (Human) | (Human) | (Human) | (Human) | | GTHYGMDV | | AKV | Philip Brouwer et al., |
| | PSGIPERFSGSNSGNTATLTISGTQAM | | | | | | | | | 2020 |
| | DEADYYCQAWGSTTAKVFGGGTKLTV | | | | | | | | | (https://science.science |
| | P | | | | | | | | | mag.org/content/early/ |
| | | | | | | | | | | 5902) |
| CoVA1- | DIQLTQSPATLSLSPGERATLSCRASQS | IGHV1-69 | IGHJ4 | IGKV3-11 | IGKJ4 | 2730 | ARVGAYDSSG | 3932 | QQRSNWP | 2020/06/15/science.abc |
| 07 | VSSYLAWYQQKPGQAPRLLIYDASNR | (Human) | (Human) | (Human) | (Human) | | YSNDY | | PRVT | Philip Brouwer et al., |
| | ATGIPARFSGSGSGTDFTLTISSLEPEDF | | | | | | | | | 2020 |
| | AVYYCQQRSNWPPRVTFGGGTKVEIK | | | | | | | | | (https://science.science |
| | | | | | | | | | | mag.org/content/early/ |
| | | | | | | | | | | 5902) |
| CoVA1- | QSVLTQPASVSGSPGQSITISCTGTSSD | IGHV3-30 | IGHJ4 | IGLV2-14 | IGLJ3 | 2731 | AREDYYDSSG | 3933 | SSYTSSSTR | Philip Brouwer et al., |
| 08 | VGGYNYVSWYQQHPGKAPKLMIYDV | (Human) | (Human) | (Human) | (Human) | | SFDY | | HWV | 2020 |
| | SNRPSGVSNRFSGSKSGNTASLTISGL | | | | | | | | | (https://science.science |
| | QAEDEADYYCSSYTSSSTRHWVFGGG | | | | | | | | | mag.org/content/early/ |
| | TKLTVL | | | | | | | | | 2020/06/15/science.abc |
| | | | | | | | | | | 5902) |
| CoVA1- | DIQMTQSPSSLSASVRDRVTITCRASQ | IGHV4-59 | IGHJ3 | IGKV1-39 | IGKJ1 | 2732 | ARHSQGWLQ | 3934 | QQSYSTPY | Philip Brouwer et al., |
| 09 | SITSSLNWYQQKPGKAPKLLIYAASSLQ | (Human) | (Human) | (Human) | (Human) | | QAVAFDI | | T | 2020 |
| | SGVPSRFSGSGSGTDFTLTISSLQPEDF | | | | | | | | | (https://science.science |
| | ATYYCQQSYSTPYTFGQGTKVEIK | | | | | | | | | mag.org/content/early/ |
| | | | | | | | | | | 2020/06/15/science.abc |
| | | | | | | | | | | 5902) |
| CoVA1- | NFMLTQPASVSGSPGQSITISCTGSSS | IGHV3-66 | IGHJ4 | IGLV2-14 | IGLJ3 | 2733 | ARGGYYYDPS | 3935 | SAYTTTSTS | Philip Brouwer et al., |
| 10 | DIAPYTFVSWYQQHSGTAPKLIIYDVR | (Human) | (Human) | (Human) | (Human) | | GYYSRSFSFDY | | WV | 2020 |
| | NRPSGISDRFSGSRSGNTASLSISGLQA | | | | | | | | | (https://science.science |
| | EDEADYYCSAYTTTSTSWVFGGGTKLT | | | | | | | | | mag.org/content/early/ |
| | VL | | | | | | | | | 2020/06/15/science.abc |
| | | | | | | | | | | 5902) |
| CoVA1- | QSALTQPPSASGSPGQSVTISCTGTSS | IGHV1-2 | IGHJ6 | IGLV2-8 | IGLJ3 | 2734 | ARDLVWATV | 3936 | SSYVGNN | Philip Brouwer et al., |
| 12 | DIGTYNYVSWYQQHPGKAPKLMIYEV | (Human) | (Human) | (Human) | (Human) | | SGTMDV | | NWV | 2020 |
| | TKRPSGVPDRFSGSKSGNTASLTVSGL | | | | | | | | | (https://science.science |
| | QADDEGDYYCSSYVGNNNWVFGGG | | | | | | | | | mag.org/content/early/ |
| | TKLTVL | | | | | | | | | 2020/06/15/science.abc |
| | | | | | | | | | | 5902) |
| CoVA1- | DIQLTQSPSSLSASVGDRVTITCQASQ | IGHV1-46 | IGHJ1 | IGKV1-33 | IGKJ4 | 2735 | ARPPRNYYDR | 3937 | QQYDNPP | Philip Brouwer et al., |
| 16 | DISNYLNWYQQRPGKAPKLLIYDASNL | (Human) | (Human) | (Human) | (Human) | | SGYYQRAEYF | | LT | 2020 |
| | ETGVPSRFSGSGSGTDFTFTISSLQPED | | | | | | QH | | | (https://science.science |
| | IATYYCQQYDNPPLTFGGGTKLEIK | | | | | | | | | mag.org/content/early/ |
| | | | | | | | | | | 2020/06/15/science.abc |
| | | | | | | | | | | 5902), Hejun Liu et al |
| | | | | | | | | | | 2020 |
| | | | | | | | | | | (https://www.biorxiv.or |
| | | | | | | | | | | g/content/10.1101/202 |
| | | | | | | | | | | 0.08.02.233536v1.full. |
| | | | | | | | | | | pdf) |
| CoVA1- | QSALTQEPSLTVSPGGTVTLTCGSSTG | IGHV3-66 | IGHJ4 | IGLV7-46 | IGLJ3 | 2736 | ARVEWAAAG | 3938 | LLSYSGVW | Philip Brouwer et al., |
| 18 | AVTSGHYPYWFQQKPGQAPRTLIYDT | (Human) | (Human) | (Human) | (Human) | | TFY | | V | 2020 |
| | SNKRSWTPARFSGSLLGGKAALTLSGA | | | | | | | | | (https://science.science |
| | QPEDEAEYYCLLSYSGVWVFGGGTKL | | | | | | | | | mag.org/content/early/ |
| | TVL | | | | | | | | | 2020/06/15/science.abc |
| | | | | | | | | | | 5902) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoVA1- | Ab | SARS-CoV2 | SARS- | | SARS-CoV1, | S; non- | B-cells; SARS-CoV2 | 649 | QVQLVESGGGLVKPGGSLRLSCAASGFT | 1728 |
| 19 | | | CoV1 | | SARS-CoV2 | RBD | Human Patient | | FSSYSMNWVRQAPGKGLEWVSSISSSSS | |
| | | | | | | | | | FIYYADSVKGRFTISRDNAKNSLYLQMNS | |
| | | | | | | | | | LRAEDTAVYYCARWKSDYYDSSGYYPAA | |
| | | | | | | | | | FDIWGQRDKWSPSS | |
| CoVA1- | Ab | SARS-CoV2 | SARS- | | SARS-CoV1, | S; non- | B-cells; SARS-CoV2 | 650 | QVQLVESGGGLVKPGGSLRLSCAASGFT | 1729 |
| 20 | | | CoV1 | | SARS-CoV2 | RBD | Human Patient | | FSSYSMNWVRQAPGKGLEWVSSISSSSS | |
| | | | | | | | | | YIYYADSVKGRFTISRDDAKNSLFLQMNS | |
| | | | | | | | | | LRAEDTAVYYCAGDQNLYCSGDSCYYHY | |
| | | | | | | | | | YGMDVWGQGTVVTVSS | |
| CoVA1- | Ab | SARS-CoV2 | SARS- | SARS-CoV2 | SARS-CoV1 | S; non- | B-cells; SARS-CoV2 | 651 | EVQLVESGGGVVQPGRSLRLSCAASGFT | 1730 |
| 21 | | | CoV1 | | | RBD | Human Patient | | FSSYAMHWVRQAPGKGLEWVAVISYD | |
| | | | | | | | | | GSNKYYADSVKGRFTISRDNTKNTLYLQ | |
| | | | | | | | | | MNSLRAEDTAVYYCARDSEYYDILTGYL | |
| | | | | | | | | | APTHYYYYYMDVWGKGTTVTVSS | |
| CoVA1- | Ab | SARS-CoV2 | SARS- | | SARS-CoV1, | S; non- | B-cells; SARS-CoV2 | 652 | EVQLVESGAEVKKPGASVKVSCKASGYT | 1731 |
| 22 | | | CoV1 | | SARS-CoV2 | RBD | Human Patient | | FTSYGISWVRQAPGQGLEWMGWISAY | |
| | | | | | | | | | NGYTNSAQKLQGRVTMITTDTSTSTAYM | |
| | | | | | | | | | ELRSLRSDDTAVYYCARDLVDTAMVQTL | |
| | | | | | | | | | DDYGMDVWGQGTMVTVSS | |
| CoVA1- | Ab | SARS-CoV2 | SARS- | | SARS-CoV1, | S; non- | B-cells; SARS-CoV2 | 653 | QVQLVQSGAEVKKPGESLKISCKGSGYR | 1732 |
| 23 | | | CoV1 | | SARS-CoV2 | RBD | Human Patient | | FTTYWIGWVRQMPGKGLEWMGIIYPG | |
| | | | | | | | | | DSDTRYSPSFQGQVTISADKSISTAYLQW | |
| | | | | | | | | | SSLTASDTAIYYCARYYYDSRGYTSIDFW | |
| | | | | | | | | | GQGTLVTVSS | |
| CoVA1- | Ab | SARS-CoV2 | SARS- | | SARS-CoV1, | S; non- | B-cells; SARS-CoV2 | 654 | EVQLVESGPGLVKPSETLSLTCTVSGGSI | 1733 |
| 25 | | | CoV1 | | SARS-CoV2 | RBD | Human Patient | | SSTSYYWGWIRQPPGKGLECIGSIYYSGS | |
| | | | | | | | | | TYYNPSLKSRVTISVDTSKNQFSLKLSSVT | |
| | | | | | | | | | AADTAVYYCARLNYDFWSGYYSYALYY | |
| | | | | | | | | | MDVWGKGTMVTVSS | |
| CoVA1- | Ab | SARS-CoV2 | SARS- | | SARS-CoV1, | S; non- | B-cells; SARS-CoV2 | 655 | QLQLQESGPGLVKPSQTLSLTCTVSGGSI | 1734 |
| 26 | | | CoV1 | | SARS-CoV2 | RBC | Human Patient | | SSGGYYWSWIRQLPGKGLEWIGYIYYSG | |
| | | | | | | | | | STYYNPSLKSRVTISVDTSKNQFSLNLSSV | |
| | | | | | | | | | TAADTAVYYCARQQLDYYDSSGCFDYW | |
| | | | | | | | | | GQGTLVTVSS | |
| CoVA1- | Ab | SARS-CoV1, | | | SARS-CoV1, | S; non- | B-cells; SARS-CoV2 | 656 | EVQLVESGPGLVKPSETLSLTCTVSGGSI | 1735 |
| 27 | | SARS-CoV2 | | | SARS-CoV2 | RBD | Human Patient | | SSYYWSWIRQPPGKRLEWIGYIYYSGST | |
| | | | | | | | | | NYNPSLKSRVTISVDTSKNQFSLKLNSVT | |
| | | | | | | | | | AADTAAYYCARGFDYWGQGTRVTVSS | |
| CoVA2- | Ab | SARS-CoV2 | SARS- | | SARS-CoV1, | S; RBD | B-cells; SARS-CoV2 | 657 | EVQLVESGGGLVQPGGSLRLSCAASGFT | 1736 |
| 01 | | | CoV1 | | SARS-CoV2 | | Human Patient | | FSSYDMHWVRQTTGKGLQWVSAIGTA | |
| | | | | | | | | | GDTYYPGSVKGRFTISRENAKNSLYLQM | |
| | | | | | | | | | NSLRAGDTAVYYCARGGDRYPVGYFDL | |
| | | | | | | | | | WGRGTLVTVSS | |
| CoVA2- | Ab | SARS-CoV1, | | SARS-CoV1, | | S; RBD | B-cells; SARS-CoV2 | 658 | VQLQQESGPGLVKPSETLSLTCTVSGGSI | 1737 |
| 02 | | SARS-CoV2 | | SARS-CoV2 | | | Human Patient | | SSSSYYWGWIRQPPGKGLEWIGSIYYSG | |
| | | | | (weak) | | | | | STYYNPSLKSRVTISVDTSKNQFSLKLSSV | |
| | | | | | | | | | TAADTAVYYCARRSTSRWGYYYMDVW | |
| | | | | | | | | | GKGTRVTVSS | |
| CoVA2- | Ab | SARS-CoV2 | SARS- | | SARS-CoV1, | S; non- | B-cells; SARS-CoV2 | 659 | EVQLVESGGGLVQPGGSLRLSCAASGFT | 1738 |
| 03 | | | CoV1 | | SARS-CoV2 | RBD | Human Patient | | FSSYSMNWVRQAPGKGLEWVSYISSSSS | |
| | | | | | | | | | TIYYADSVKGRFTISRDNAKNSLYLQMNS | |
| | | | | | | | | | LRAEDTAVYYCAREANSDFWSGYLGYFD | |
| | | | | | | | | | YWGQGTLVTVSS | |
| CoVA2- | Ab | SARS-CoV2 | SARS- | SARS-CoV2 | SARS-CoV1 | S; RBD | B-cells; SARS-CoV2 | 660 | QVQLVETGGGLIQPGGSLRLSCAASGFT | 1739 |
| 04 | | | CoV1 | | | | Human Patient | | VSSNYMSWVRQAPGKGLEWVSVIYSG | |
| | | | | | | | | | GSTFYADSVKGRFTISRDNSKNTLYLQM | |
| | | | | | | | | | NSLRAEDTAVYYCARDLERAGGMDVW | |
| | | | | | | | | | GQGTMVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoVA1- | EIVMTQSPATLSVSPGERATLSCRASQ | IGHV3-21 | IGHJ3 | IGKV3-15 | IGKJ1 | 2737 | ARWKSDYYD | 3939 | QQYNNWP | Philip Brouwer et al., |
| 19 | SVSSNLAWYQQKPGQAPRLLIYGAST | (Human) | (Human) | (Human) | (Human) | | SSGYYPAAFDI | | PWT | 2020 |
| | RATGIPARFSGSGSGTEFTLTISSLQSE | | | | | | | | | (https://science.science |
| | DFAVYYCQQYNNWPPWTFGQGTKV | | | | | | | | | mag.org/content/early/ |
| | EIK | | | | | | | | | 2020/06/15/science.abc |
| | | | | | | | | | | 5902) |
| CoVA1- | NFMLTQPPSVSVAPGKTARITCGGNN | IGHV3-21 | IGHJ6 | IGLV3-21 | IGLJ3 | 2738 | AGDQNLYCS | 3940 | QVWDSSS | Philip Brouwer et al., |
| 20 | IGSKSVHWYQQKPGQAPVLVIYYDSD | (Human) | (Human) | (Human) | (Human) | | GDSCYYHYYG | | DHWV | 2020 |
| | RPSGIPERFSGSNSGNTATLTISRVEAG | | | | | | MDV | | | (https://science.science |
| | DEADYYCQVWDSSSDHWVFGGGTKL | | | | | | | | | mag.org/content/early/ |
| | TVL | | | | | | | | | 2020/06/15/science.abc |
| | | | | | | | | | | 5902) |
| CoVA1- | EIVMTQSPATLSVSPGERATLSYRASQ | IGHV3-30 | IGHJ6 | IGKV3-15 | IGKJ2 | 2739 | ARDSEYYDILT | 3941 | QQYNNWP | Philip Brouwer et al., |
| 21 | SVSSNLAWYQQKPGQAPRLLIYGAST | (Human) | (Human) | (Human) | (Human) | | GYLAPTHYYY | | PGT | 2020 |
| | RATGIPARFSGSGSGTEFTLTISSLQSE | | | | | | YYMDV | | | (https://science.science |
| | DFAVYYCQQYNNWPPGTFGQGTKLEI | | | | | | | | | mag.org/content/early/ |
| | K | | | | | | | | | 2020/06/15/science.abc |
| | | | | | | | | | | 5902) |
| CoVA1- | SYELTQPPSVSVSPGQTASITCSGDKLG | IGHV1-18 | IGHJ6 | IGLV3-1 | IGLJ3 | 2740 | ARDLVDTAM | 3942 | QAWDSST | Philip Brouwer et al., |
| 22 | DKYACWYQQKPGQSPVLVTYQDNKR | (Human) | (Human) | (Human) | (Human) | | VQTLDDYGM | | AV | 2020 |
| | PSGIPERFSGSNSGNTATLTISGTQAM | | | | | | DV | | | (https://science.science |
| | DEADYYCQAWDSSTAVFGGGTKLTVL | | | | | | | | | mag.org/content/early/ |
| | | | | | | | | | | 2020/06/15/science.abc |
| | | | | | | | | | | 5902) |
| CoVA1- | SYELTQPPSVSVSPGQTARITCSGDALP | IGHV5-51 | IGHJ4 | IGLV3-25 | IGLJ3 | 2741 | ARYYYDSRGY | 3943 | QSADSSG | Philip Brouwer et al., |
| 23 | KQYAYWYQQKPGQAPVLVIYKDSERP | (Human) | (Human) | (Human) | (Human) | | TSIDF | | TYSVV | 2020 |
| | SGIPERFSGSSSGTTVTLTISGVQAEDE | | | | | | | | | (https://science.science |
| | ADYYCQSADSSGTYSVVFGGGTKLTVL | | | | | | | | | mag.org/content/early/ |
| | | | | | | | | | | 2020/06/15/science.abc |
| | | | | | | | | | | 5902) |
| CoVA1- | DIQLTQSPSTLSASVGDRVTITCRASQS | IGHV4-39 | IGHJ6 | IGKV1-5 | IGKJ3 | 2742 | ARLNYDFWS | 3944 | QQYNSYS | Philip Brouwer et al., |
| 25 | ISSWLAWYQQKPGKAPKLLIYKASSLE | (Human) | (Human) | (Human) | (Human) | | GYYSYALYYM | | IT | 2020 |
| | SGVPSRFSGSGSGTEFTLTISSLQPDDF | | | | | | DV | | | (https://science.science |
| | ATYYCQQYNSYSITFGPGTKVDIK | | | | | | | | | mag.org/content/early/ |
| | | | | | | | | | | 2020/06/15/science.abc |
| | | | | | | | | | | 5902) |
| CoVA1- | DIVMTQSPSFLSASVGDRVTITCRASQ | IGHV4-31 | IGHJ4 | IGKV1-9 | IGKJ4 | 2743 | ARQQLDYYDS | 3945 | QQLHSY | Philip Brouwer et al., |
| 26 | GISSYLAWYQQKPGKAPKLLIYAASTL | (Human) | (Human) | (Human) | (Human) | | SGCFDY | | PLT | 2020 |
| | QSGVPSRFSGSGSGTEFTLTISSLQPED | | | | | | | | | (https://science.science |
| | FATYYCQQLHSYPLTFGGGTKVDIK | | | | | | | | | mag.org/content/early/ |
| | | | | | | | | | | 2020/06/15/science.abc |
| | | | | | | | | | | 5902) |
| CoVA1- | DVVMTQSPGTLSLSPGERATLSCRAS | IGHV4-59 | IGHJ4 | IGKV3-20 | IGKJ3 | 2744 | ARAMGSYRS | 3946 | QQYGSS | Philip Brouwer et al., |
| 27 | QNVSSSYLAWYQQKPGQAPRLLIYGA | (Human) | (Human) | (Human) | (Human) | | PFDY | | PLFT | 2020 |
| | SSRATGIPDRFSGSGSGTDFTLTISRLEP | | | | | | | | | (https://science.science |
| | EDFAVYYCQQYGSSPLFTFGPGTKVEI | | | | | | | | | mag.org/content/early/ |
| | K | | | | | | | | | 2020/06/15/science.abc |
| | | | | | | | | | | 5902) |
| CoVA2- | DIVMTQSPSSLSASVGDRVTITCRASQ | IGHV3-13 | IGHJ2 | IGKV1-39 | IGKJ4 | 2745 | ARGGDRYPV | 3947 | QQSYST | Philip Brouwer et al., |
| 01 | SISSYLNWYQQKPGKAPKLLIYAASSLQ | (Human) | (Human) | (Human) | (Human) | | GYFDL | | PPVT | 2020 |
| | SGVPSRFSGSGSGTDFTLTISSLQPEDF | | | | | | | | | (https://science.science |
| | ATYYCQQSYSTPPVTFGGGTKVEIK | | | | | | | | | mag.org/content/early/ |
| | | | | | | | | | | 2020/06/15/science.abc |
| | | | | | | | | | | 5902) |
| CoVA2- | DIVMTQSPSSLSASVGDRVTITCRASQ | IGHV4-39 | IGHJ6 | IGKV1-39 | IGKJ3 | 2746 | ARRSTSRWG | 3948 | QQSYSTH | Philip Brouwer et al., |
| 02 | SISNYLNWYQQKPGKAPKLLLYAASDL | (Human) | (Human) | (Human) | (Human) | | YYYMDV | | MST | 2020 |
| | QSGVPSRFSGSGSGTDFTLTISSLQPED | | | | | | | | | (https://science.science |
| | FATYYCQQSYSTHMSTFGQGTKVDIK | | | | | | | | | mag.org/content/early/ |
| | | | | | | | | | | 2020/06/15/science.abc |
| | | | | | | | | | | 5902) |
| CoVA2- | EIVMTQSPATLSLSPGERATLSCRASQS | IGHV3-48 | IGHJ4 | IGKV3-11 | IGKJ5 | 2747 | AREANSDFW | 3949 | QQRSNWP | 2020/06/15/science.abc |
| 03 | VSSYLAWYQQKPGQAPRLLIYDASNR | (Human) | (Human) | (Human) | (Human) | | SGYLGYFDY | | QVT | Philip Brouwer et al., |
| | ATGIPARFSGSGSGTDFTLTISSLEPEDF | | | | | | | | | 2020 |
| | AVYYCQQRSNWPQVTFGQGTRLEIK | | | | | | | | | (https://science.science |
| | | | | | | | | | | mag.org/content/early/ |
| | | | | | | | | | | 5902) |
| CoVA2- | EIVMITQSPGTLSLSPGERATLSCRASQ | IGHV3-53 | IGHJ6 | IGKV3-20 | IGKJ3 | 2748 | ARDLERAGG | 3950 | QQYGSLY | Philip Brouwer et al., |
| 04 | SVSSSYLAWYQQKPGQAPRLLIYGASS | (Human) | (Human) | (Human) | (Human) | | MDV | | T | 2020 |
| | RATGIPDRFSGSGSGTDFTLTISRLEPE | | | | | | | | | (https://science.science |
| | DFAVYYCQQYGSLYTFGQGTKVDIK | | | | | | | | | mag.org/content/early/ |
| | | | | | | | | | | 2020/06/15/science.abc |
| | | | | | | | | | | 5902); Nicholas Wu |
| | | | | | | | | | | (https://www.biorxiv.or |
| | | | | | | | | | | g/content/10.1101/202 |
| | | | | | | | | | | 0.07.26.222232v1) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoVA2- | Ab | SARS-CoV2 | SARS- | | SARS-CoV1, | S; RBD | B-cells; SARS-CoV2 | 661 | EVQLLESGAEVKKPGESLKISCKGSGYSF | 1740 |
| 05 | | | CoV1 | | SARS-CoV2 | | Human Patient | | TSYWIGWVRQMPGKGLEWMGIIYPGD | |
| | | | | | | | | | SDTRYSPSFQGQVTISADKSISTAYLQWS | |
| | | | | | | | | | SLKASDTAMYYCARHMRPSIAARPGYQ | |
| | | | | | | | | | YYMDVWGKGTRVTVSS | |
| CoVA2- | Ab | SARS-CoV2 | SARS- | SARS-CoV2 | SARS-CoV1 | S; RBD | B-cells; SARS-CoV2 | 662 | EVQLVETGGGLIQPGGSLRLSCAASGLT | 1741 |
| 07 | | | CoV1 | | | | Human Patient | | VSSNYMNWVRQAPGKGLEWVSVIYSG | |
| | | | | | | | | | GSTYYADSVKGRFTISRDNSKNTLYLQM | |
| | | | | | | | | | NSLRAEDTAVYYCAREAYGMDVWGQG | |
| | | | | | | | | | TMVTVSS | |
| CoVA2- | Ab | SARS-CoV2 | SARS- | | SARS-CoV1, | S; non- | B-cells; SARS-CoV2 | 663 | EVQLLESGGGLVQPGGSLKLSCAASGFT | 1742 |
| 10 | | | CoV1 | | SARS-CoV2 | RBD | Human Patient | | FSSYAMSWVRQAPGKGLEWVSAISGSG | |
| | | | | | | | | | SNTYYADSVKGRFTISRDNSKNTLYLQM | |
| | | | | | | | | | NSLRAEDTAVYYCAKGLRGQQLVIPTEY | |
| | | | | | | | | | FQHWGQGTLVTVSS | |
| CoVA2- | Ab | SARS-CoV2 | SARS- | SARS-CoV2 | SARS-CoV1 | S; RBD | B-cells; SARS-CoV2 | 664 | EVQLVESGAEVKKPGSSVKVSCKASGGT | 1743 |
| 11 | | | CoV1 | (weak) | | | Human Patient | | LSSYAISWVRQAPGQGLEWMGGIIPIFG | |
| | | | | | | | | | TANYAQKFQGRVTITTDESTSTAYMELSS | |
| | | | | | | | | | LRSEDTAVYYCARGPRGCSSTSCYGSYFD | |
| | | | | | | | | | YWGQGTLVTVSS | |
| CoVA2- | Ab | SARS-CoV2 | SARS- | | SARS-CoV1, | S; non- | B-cells; SARS-CoV2 | 665 | EVQLVESGGGLVKPGGSLRLSCAASGFT | 1744 |
| 12 | | | CoV1 | | SARS-CoV2 | RBD | Human Patient | | FGSYSMSWVRQAPGKGLEWVSSISRSS | |
| | | | | | | | | | SYIYNADSVRGRLTISRDNAKNSLYLQM | |
| | | | | | | | | | NSLRVEDTAVYYCARDQPLPDILTGYYT | |
| | | | | | | | | | GPLDYWGQGTLVTVSS | |
| CoVA2- | Ab | SARS-CoV2 | SARS- | SARS-CoV2 | SARS-CoV1 | S; RBD | B-cells; SARS-CoV2 | 666 | QVQLVETGGGLIQPGGSLRLSCAASGFT | 1745 |
| 13 | | | CoV1 | (weak) | | | Human Patient | | VSSNYMSWVRQAPGKGLEWVSVIYSG | |
| | | | | | | | | | GSTYYADSVKGRFTISRDNSKNTLYLQM | |
| | | | | | | | | | NSLRAEDTAVYYCARDLDTMGGMDVW | |
| | | | | | | | | | GQGTRVTVSS | |
| CoVA2- | Ab | SARS-CoV1, | | | SARS-CoV1, | S; non- | B-cells; SARS-CoV2 | 667 | QVQLVQSGAEVKKPGSSVKVSCKASGG | 1746 |
| 14 | | SARS-CoV2 | | | SARS-CoV2 | RBD | Human Patient | | TFSSYAIIWVRQAPGQGLEWMGGIIPIF | |
| | | | | | | | | | GTANYAQKFQGRVTITTDESTSTAYMEL | |
| | | | | | | | | | SSLRSEDTAVYYCARVRYYDSSGYYEDY | |
| | | | | | | | | | WGQGTLVTVSS | |
| CoVA2- | Ab | SARS-CoV2 | SARS- | SARS-CoV2 | SARS-CoV1 | S; RBD | B-cells; SARS-CoV2 | 668 | QVQLLESGGGLVQPGGSLRLSCAASGFT | 1747 |
| 15 | | | CoV1 | | | | Human Patient | | FSSYAMSWVRQAPGKGLEWASAISGSG | |
| | | | | | | | | | GSTYYADSVEGRFTISRDNSKNTLYLQM | |
| | | | | | | | | | NSLRAEDTAVYYCAKDTGYCGDDCYIKLI | |
| | | | | | | | | | RGGPDYWGQGTLVTVSS | |
| CoVA2- | Ab | SARS-CoV2 | SARS- | | SARS-CoV1, | S; RBD | B-cells; SARS-CoV2 | 669 | QVQLVQSGAEVKKPGATVKISCKVSGYT | 1748 |
| 16 | | | CoV1 | | SARS-CoV2 | | Human Patient | | FTDYYMHWVQQAPGKGLEWMGLVDP | |
| | | | | | | | | | EDGETIYAEKFQGRVTITADTSTDTAYM | |
| | | | | | | | | | ELSSLRSEDTAVYYCASSDSSGFVGSRGF | |
| | | | | | | | | | DYWGQGTRVTVSS | |
| CoVA2- | Ab | SARS-CoV2 | SARS- | | SARS-CoV1, | S; RBD | B-cells; SARS-CoV2 | 670 | QVQLVESGAEVKKPGSSVKVSCKASGGT | 1749 |
| 17 | | | CoV1 | | SARS-CoV2 | | Human Patient | | FSSYGINWVRQAPGQGLEWMGGIIPIF | |
| | | | | | | | | | GTANYAQKFQGRVTITTDESTSTAYMEL | |
| | | | | | | | | | SSLRSEDTAVYYCASFGDDSGDEGVRW | |
| | | | | | | | | | GQGTLVTVSS | |
| CoVA2- | Ab | SARS-CoV1, | | SARS-CoV2 | SARS-CoV1 | S; non- | B-cells; SARS-CoV2 | 671 | EVQLVQSGAEVKKPGSSVKVSCKASGGT | 1750 |
| 18 | | SARS-CoV2 | | | | RBD | Human Patient | | FSSYAISWVRQAPGQGLEWMGGIIPIFG | |
| | | | | | | | | | TTNYAQKFQGRVTITTDESTSTAYMELSS | |
| | | | | | | | | | LRSEDTAVYYCARVYSYDSSGYYLEYWG | |
| | | | | | | | | | QGTRVTVSS | |
| CoVA2- | Ab | SARS-CoV2 | SARS- | | SARS-CoV1, | S; RBD | B-cells; SARS-CoV2 | 672 | EVQLVESGGGLIQPGGSLRLSCAASGFT | 1751 |
| 20 | | | CoV1 | | SARS-CoV2 | | Human Patient | | VSSNYMSWVRQAPGKGLEWVSVIYSG | |
| | | | | | | | | | GSTYYADSVKGRFTISRDNSKNTLYLQM | |
| | | | | | | | | | NSLRAEDTAVYYCASPLLLTPPDYYYYMD | |
| | | | | | | | | | VWGKGTMVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoVA2- | DIQMTQSPSSLSASVGDRVTITCQASQ | IGHV5-51 | IGHJ6 | IGKV1-33 | IGKJ4 | 2749 | ARHMRPSIAA | 3951 | QQYDNLPL | Philip Brouwer et al., |
| 05 | DISNYLNWYQQKPGKAPKLLIYDASNL | (Human) | (Human) | (Human) | (Human) | | RPGYQYYMD | | T | 2020 |
| | ETGVPSRFSGSGSGTDFTFTISSLQPED | | | | | | V | | | (https://science.science |
| | IATYYCQQYDNLPLTFGGGTKLEIK | | | | | | | | | mag.org/content/early/ |
| | | | | | | | | | | 2020/06/15/science.abc |
| | | | | | | | | | | 5902) |
| CoVA2- | EIVLTQSPGTLSLSPGERATLSCRASQS | IGHV3-53 | IGHJ6 | IGKV3-20 | IGKJ3 | 2750 | AREAYGMDV | 3952 | QQYGSSP | Philip Brouwer et al., |
| 07 | VSSSYLAWYQQKPGQAPRLLIYGASSR | (Human) | (Human) | (Human) | (Human) | | | | GT | 2020 |
| | ATGIPDRFSGSWSGTDFTLTISRLEPED | | | | | | | | | (https://science.science |
| | FAVYYCQQYGSSPGTFGQGTKVDIK | | | | | | | | | mag.org/content/early/ |
| | | | | | | | | | | 2020/06/15/science.abc |
| | | | | | | | | | | 5902) |
| CoVA2- | DIVMTQSPGTLSLSPGERATLSCRASQ | IGHV3-23 | IGHJ1 | IGKV3-20 | IGKJ4 | 2751 | AKGLRGQQL | 3953 | QQYGSSLL | Philip Brouwer et al., |
| 10 | SVSSSYLAWYQQKPGQAPRLLIYGASS | (Human) | (Human) | (Human) | (Human) | | VIPTEYFQH | | T | 2020 |
| | RATGIPDRFSGSGSGTDFTLTISRLEPE | | | | | | | | | (https://science.science |
| | DFAVYYCQQYGSSLLTFGGGTKVEIK | | | | | | | | | mag.org/content/early/ |
| | | | | | | | | | | 2020/06/15/science.abc |
| | | | | | | | | | | 5902) |
| CoVA2- | DIQLTQSPGTLSLSPGERATLSCRASQS | IGHV1-69 | IGHJ4 | IGKV3-20 | IGKJ4 | 2752 | ARGPRGCSST | 3954 | QQYGSSPR | Philip Brouwer et al., |
| 11 | VSSSYLAWYQQKPGQAPRLLIYGASSR | (Human) | (Human) | (Human) | (Human) | | SCYGSYFDY | | LT | 2020 |
| | ATGIPDRFSGSGSGTDFTLTISRLEPED | | | | | | | | | (https://science.science |
| | FAVYYCQQYGSSPRLTFGGGTKVDIK | | | | | | | | | mag.org/content/early/ |
| | | | | | | | | | | 2020/06/15/science.abc |
| | | | | | | | | | | 5902) |
| CoVA2- | EIVLTQSPATLSVSPGERATLSCRASQS | IGHV3-21 | IGHJ4 | IGKV3-15 | IGKJ1 | 2753 | ARDQPLPDIL | 3955 | QQYNNWP | Philip Brouwer et al., |
| 12 | VSSSLAWYQQKPGQAPRLLIYAASTRA | (Human) | (Human) | (Human) | (Human) | | TGYYTGPLDY | | PWT | 2020 |
| | TGIPARFSGSGSGTEFTLTISSLQSEDFA | | | | | | | | | (https://science.science |
| | VYYCQQYNNWPPWTFGQGTKLEIK | | | | | | | | | mag.org/content/early/ |
| | | | | | | | | | | 2020/06/15/science.abc |
| | | | | | | | | | | 5902) |
| CoVA2- | EIVMTQSPGTLSLSPGERATLSCRASQ | IGHV3-53 | IGHJ6 | IGKV3-20 | IGKJ1 | 2754 | ARDLDTMGG | 3956 | QQYGSSP | Philip Brouwer et al., |
| 13 | SVSSSYLAWYQQKPGQAPRLLIYGASS | (Human) | (Human) | (Human) | (Human) | | MDV | | GT | 2020 |
| | RATGIPDRFSGSGSGTDFTLTISRLEPE | | | | | | | | | (https://science.science |
| | DFAVYYCQQYGSSPGTFGQGTKVEIK | | | | | | | | | mag.org/content/early/ |
| | | | | | | | | | | 2020/06/15/science.abc |
| | | | | | | | | | | 5902) |
| CoVA2- | EIVLTQSPATLSLSPGERATLSCRASQS | IGHV1-69 | IGHJ4 | IGKV3-11 | IGKJ1 | 2755 | ARVRYYDSSG | 3957 | QQRSNWP | Philip Brouwer et al., |
| 14 | VSSYLAWYQQEPGQAPRLLIYDASNR | (Human) | (Human) | (Human) | (Human) | | YYEDY | | PMYT | 2020 |
| | ATGIPARFSGSGSGTDFTLTISSLEPEDF | | | | | | | | | (https://science.science |
| | AVYYCQQRSNWPPMYTFGQGTKVEI | | | | | | | | | mag.org/content/early/ |
| | K | | | | | | | | | 2020/06/15/science.abc |
| | | | | | | | | | | 5902) |
| CoVA2- | DIVMTQSPLSLPVTLGQPASISCRSSQS | IGHV3-23 | IGHJ4 | IGKV2-30 | IGKJ2 | 2756 | AKDTGYCGD | 3958 | MQGTHW | Philip Brouwer et al., |
| 15 | LVYSDGNTFLNWFQQRPGQSPRRLIY | (Human) | (Human) | (Human) | (Human) | | DCYIKLIRGG | | PRT | 2020 |
| | QVSNRDSGVPDRFSGSGSGTDFTLKIS | | | | | | PDY | | | (https://science.science |
| | RVEAEDVGVYYCMQGTHWPRTFGQ | | | | | | | | | mag.org/content/early/ |
| | GTKLEIK | | | | | | | | | 2020/06/15/science.abc |
| | | | | | | | | | | 5902) |
| CoVA2- | DIQLTQSPSSLSASVGDRVTITCRASQG | IGHV1-69 | IGHJ4 | IGKV1-17 | IGKJ1 | 2757 | ASSDSSGFVG | 3959 | LQHNSYPP | Philip Brouwer et al., |
| 16 | IRNDLGWYQQKPGKAPKRLIYAASSL | (Human) | (Human) | (Human) | (Human) | | SRGFDY | | L | 2020 |
| | QSGVPSRFSGSGSGTEFTLTISSLQPED | | | | | | | | | (https://science.science |
| | FATYYCLQHNSYPPLFGQGTKVEIK | | | | | | | | | mag.org/content/early/ |
| | | | | | | | | | | 2020/06/15/science.abc |
| | | | | | | | | | | 5902) |
| CoVA2- | EIVMTQSPATLSLSPGERATLSCRASQS | IGHV1-69 | IGHJ4 | IGKV3-11 | IGKJ1 | 2758 | ASFGDDSGDE | 3960 | QQRSNWP | Philip Brouwer et al., |
| 17 | VSSYLAWYQQKPGQAPRLLIYDASNR | (Human) | (Human) | (Human) | (Human) | | GVR | | PYT | 2020 |
| | ATGIPARFSGSGSGTDFTLTISSLEPEDF | | | | | | | | | (https://science.science |
| | AVYYCQQRSNWPPYTFGQGTKVEIK | | | | | | | | | mag.org/content/early/ |
| | | | | | | | | | | 2020/06/15/science.abc |
| | | | | | | | | | | 5902) |
| CoVA2- | EIVLTQSPATLSLSPGERATLSCRASQS | IGHV1-69 | IGHJ4 | IGKV3-11 | IGKJ5 | 2759 | ARVYSYDSSG | 3961 | QQRSNWP | Philip Brouwer et al., |
| 18 | VSSYLAWYQQKPGQAPRLLIYDASNR | (Human) | (Human) | (Human) | (Human) | | YYLEY | | PSIT | 2020 |
| | ATGIPARFSGSGSGTDFTLTISSLEPEDF | | | | | | | | | (https://science.science |
| | AVYYCQQRSNWPPSITFGQGTRLEIK | | | | | | | | | mag.org/content/early/ |
| | | | | | | | | | | 2020/06/15/science.abc |
| | | | | | | | | | | 5902) |
| CoVA2- | DVVMTQSPSSLSVSVGDRVTITCRASQ | IGHV3-53 | IGHJ6 | IGKV1-17 | IGKJ1 | 2760 | ASPLLLTPPD | 3962 | LQHNSYL | Philip Brouwer et al., |
| 20 | GIRNDLGWYQQKPGKAPKRLIYAASSL | (Human) | (Human) | (Human) | (Human) | | YYYYMDV | | WT | 2020 |
| | QSGVPSRFSGSGSGTEFTLTISSLQPED | | | | | | | | | (https://science.science |
| | FATYYCLQHNSYLWTFGQGTKLEIK | | | | | | | | | mag.org/content/early/ |
| | | | | | | | | | | 2020/06/15/science.abc |
| | | | | | | | | | | 5902) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoVA2- | Ab | SARS-CoV2 | SARS- | | SARS-CoV1, | S; non- | B-cells; SARS-CoV2 | 673 | EVQLVESGPGLVKPSETLSLTCTVSGGSI | 1752 |
| 22 | | | CoV1 | | SARS-CoV2 | RBC | Human Patient | | SSYYWSWIRQPAGKGLEWIGRIYTSGST | |
| | | | | | | | | | NYNPSLKSRVTMSVDTSKNQFSLKLSSV | |
| | | | | | | | | | TAADTAVYYCARWKYNDRFDYWGQGT | |
| | | | | | | | | | RVTVSS | |
| CoVA2- | Ab | SARS-CoV2 | SARS- | | SARS-CoV1, | S; RBD | B-cells; SARS-CoV2 | 674 | EVQLVESGAEVKKPGSSVKVSCKASGGT | 1753 |
| 23 | | | CoV1 | | SARS-CoV2 | | Human Patient | | FSSYAISWVRQAPGQGLEWMGGIIPIFG | |
| | | | | | | | | | TANYAQKFQGRVTITTDESTSTAYMELSS | |
| | | | | | | | | | LRSEDTAVYYCARGPRYCSSTSCYAGVYF | |
| | | | | | | | | | DYWGQGTRVTVSS | |
| CoVA2- | Ab | SARS-CoV2 | SARS- | | SARS-CoV1, | S; RBD | B-cells; SARS-CoV2 | 675 | EVQLVQSGAEVKKPGESLRISCKGSGYSF | 1754 |
| 24 | | | CoV1 | | SARS-CoV2 | | Human Patient | | TSYWISWVRQMPGKGLEWMGRIDPSD | |
| | | | | | | | | | SYTNYSPSFQGHVTISADKSISTAYLQWS | |
| | | | | | | | | | SLKASDTAMYYCARPNPAGGYDSSGWV | |
| | | | | | | | | | DAFDIWGQGTRVTVSS | |
| CoVA2- | Ab | SARS-CoV2 | SARS- | | SARS-CoV1, | S; non- | B-cells; SARS-CoV2 | 676 | EVQLVESGAEVKKPGASVKVSCKVSGYT | 1755 |
| 25 | | | CoV1 | | SARS-CoV2 | RBC | Human Patient | | LPELSMHWVRQTPGKGLEWMGGFDP | |
| | | | | | | | | | EDGETIYAQKFQGRVTMTEDTSTDTAY | |
| | | | | | | | | | MELSSLRSEDTAVYYCATGPTIAAAATN | |
| | | | | | | | | | WFDPGGQGTLVTVSS | |
| CoVA2- | Ab | SARS-CoV2 | SARS- | | SARS-CoV1, | S; non- | B-cells; SARS-CoV2 | 577 | EVQLVESGGGLVKPGGSLRLSCAASGFT | 1756 |
| 26 | | | CoV1 | | SARS-CoV2 | RBD | Human Patient | | FSNAWMSWVRQAPGKGLEWVGRIKSK | |
| | | | | | | | | | TDGGTTDYAAPVKGRFTISRDDSKNTLY | |
| | | | | | | | | | LQMNSLKTEDTAVYYCTTDRGDSYGYYY | |
| | | | | | | | | | CMDVWGKGTRVTVSS | |
| CoVA2- | Ab | SARS-CoV2 | SARS- | | SARS-CoV1, | S; non- | B-cells; SARS-CoV2 | 678 | EVQLVESGGGVVQPGRSLRLSCAASGFT | 1757 |
| 28 | | | CoV1 | | SARS-CoV2 | RBC | Human Patient | | FSSYGMHWVRQAPGKGLEWVAVIWY | |
| | | | | | | | | | DGSNKYYADSVKGRFTISRDNSKNTLYL | |
| | | | | | | | | | QMDSLRAEDTAMYYCAKDKAPPCSSG | |
| | | | | | | | | | WYYFDYWGQGTLVTVSS | |
| CoVA2- | Ab | SARS-CoV2 | SARS- | SARS-CoV2 | SARS-CoV1 | S; RBD | B-cells; SARS-CoV2 | 679 | EVQLVESGGGVVQPGRSLRLSCAASGFT | 1758 |
| 29 | | | CoV1 | | | | Human Patient | | FSSYTMHWVRQAPGKGLEWVAVISYD | |
| | | | | | | | | | GSNKYYADSVKGRFTISRDNSKNTLYLQ | |
| | | | | | | | | | MNSLRAEDTAVYYCASLPVVPAAIGPLP | |
| | | | | | | | | | AFDIWGQGTMVTVSS | |
| CoVA2- | Ab | SARS-CoV2 | SARS- | | SARS-CoV1, | S; non- | B-cells; SARS-CoV2 | 680 | EVQLVESGPGLVKPSQTLSLTCTVSGGSI | 1759 |
| 30 | | | CoV1 | | SARS-CoV2 | RBD | Human Patient | | SSGDYYWSWIRQPPGKGLEWIGYIYYSG | |
| | | | | | | | | | STYYNPSLKSRVTISVDTSKNQFSLKLSSV | |
| | | | | | | | | | TAADTAVYYCARGVEDPVVPAAIPWCW | |
| | | | | | | | | | FDPWGQGTRVTVSS | |
| CoVA2- | Ab | SARS-CoV2 | SARS- | | SARS-CoV1, | S; RBD | B-cells; SARS-CoV2 | 681 | EVQLVESGVEVKKPGASVKVSCKAFGYT | 1760 |
| 31 | | | CoV1 | | SARS-CoV2 | | Human Patient | | FTGQDMHWVRQAPGQGLEWMGWIN | |
| | | | | | | | | | PSSAGTNYAQKFQGRVTMTRDTSISTAY | |
| | | | | | | | | | MELSRLRSDDTSVYYCRKMLTIFGKVNQ | |
| | | | | | | | | | TMLLISGAKGQWSPSLQWGQGTMVTV | |
| | | | | | | | | | SS | |
| CoVA2- | Ab | SARS-CoV1, | | | SARS-CoV1, | S; RBD | B-cells; SARS-CoV2 | 682 | EVQLVESGAEVKKPGSSVKVSCKASGGT | 1761 |
| 32 | | SARS-CoV2 | | | SARS-CoV2 | | Human Patient | | FSSYAISWVRQAPGQGLEWMGGIIPIFG | |
| | | | | | | | | | TANYAQKFQGRATITTDESTSTAYMELSS | |
| | | | | | | | | | LGSEDTAVYYCARTHSYDNSGQYFDYW | |
| | | | | | | | | | GQGTMVTVSS | |
| CoVA2- | Ab | SARS-CoV2 | SARS- | | SARS-CoV1, | S; non- | B-cells; SARS-CoV2 | 683 | QVQLVQSGAEVKKPGESLRISCKGSGYS | 1762 |
| 33 | | | CoV1 | | SARS-CoV2 | RBC | Human Patient | | FTSHWISWVRQMPGKGLEWMGRIDPS | |
| | | | | | | | | | DSYTNYSPSFQGHVTISADKSISTAYLQW | |
| | | | | | | | | | SSLKASDTAMYYCARLKVITIFGVVRDDY | |
| | | | | | | | | | GMDVWGQGTTVTVSS | |
| CoVA2- | Ab | SARS-CoV2 | SARS- | | SARS-CoV1, | S; non- | B-cells; SARS-CoV2 | 684 | QVQLVESGGGVVQPGRSLRLSCAASGF | 1763 |
| 34 | | | CoV1 | | SARS-CoV2 | RBC | Human Patient | | TFSSYAMHWVRQAPGKGLEWVAVISY | |
| | | | | | | | | | DGSNKYYADSVKGRFTISRDNSKNTLYL | |
| | | | | | | | | | QMNSLRAEDTAVYYCARSASGSYYGAF | |
| | | | | | | | | | DYWGQGTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoVA2- | AIRMTQSPSSLSASVGDRVTITCQASQ | IGHV4-4 | IGHJ4 | IGKV1-33 | IGKJ5 | 2761 | ARGPRYCSST | 3963 | QQYDNLPI | Philip Brouwer et al., |
| 22 | DISNYLNWYQQKPGKAPKLLIYDASNL | (Human) | (Human) | (Human) | (Human) | | SCYAGVYFDY | | T | 2020 |
| | ETGVPSRFSGSGSGTDFTFTISSLQPED | | | | | | | | | (https://science.science |
| | IATYYCQQYDNLPITFGQGTRLEIK | | | | | | | | | mag.org/content/early/ |
| | | | | | | | | | | 2020/06/15/science.abc |
| | | | | | | | | | | 5902) |
| CoVA2- | EIVLTQSPGTLSLSPGERATLSCRASQS | IGHV1-69 | IGHJ4 | IGKV3-20 | IGKJ4 | 2762 | ARDGFGDVE | 3964 | QQYGSSP | Philip Brouwer et al., |
| 23 | VSSSYLAWYQQKPGQAPRLLIYGASSR | (Human) | (Human) | (Human) | (Human) | | EMATIKDAFD | | GVT | 2020 |
| | ATGIPDRFSGSGSGTDFTLTISRLEPED | | | | | | I | | | (https://science.science |
| | FAVYYCQQYGSSPGVTFGGGTKLEIK | | | | | | | | | mag.org/content/early/ |
| | | | | | | | | | | 2020/06/15/science.abc |
| | | | | | | | | | | 5902) |
| CoVA2- | AIRMTQSPSSLSASVGDRVTITCRASQ | IGHV5-10- | IGHJ3 | IGKV1-39 | IGKJ2 | 2763 | ARPNPAGGY | 3965 | QQSYSTPQ | Philip Brouwer et al., |
| 24 | SISSYLNWYQQKPGKAPKLLIYAASSLQ | 1 | (Human) | (Human) | (Human) | | DSSGWVDAF | | T | 2020 |
| | SGVPSRFSGSGSGTDFTLTISSLQPEDF | (Human) | | | | | DI | | | (https://science.science |
| | ATYYCQQSYSTPQTFGQGTKLEIK | | | | | | | | | mag.org/content/early/ |
| | | | | | | | | | | 2020/06/15/science.abc |
| | | | | | | | | | | 5902) |
| CoVA2- | QPVLTQPASVSGSPGQSITISCTGTSSD | IGHV1-24 | IGHJ5 | IGLV2-14 | IGLJ3 | 2764 | ATGPTIAAAA | 3966 | SSYTSSST | Philip Brouwer et al., |
| 25 | VGGYNYVSWYQQHPGKAPKLMIYDV | (Human) | (Human) | (Human) | (Human) | | TNWFDP | | WV | 2020 |
| | SNRPSGVSNRFSGSRSGDTASLTISGL | | | | | | | | | (https://science.science |
| | QAEDEADYYCSSYTSSSTWVFGGGTKL | | | | | | | | | mag.org/content/early/ |
| | TVL | | | | | | | | | 2020/06/15/science.abc |
| | | | | | | | | | | 5902) |
| CoVA2- | QLVLTQPPSVSVSPGQTASITCSGDKL | IGHV3-15 | IGHJ6 | IGLV3-1 | IGLJ3 | 2765 | TTDRGDSYGY | 3967 | QAWDSST | Philip Brouwer et al., |
| 26 | GDKYACWYQQKPGQSPVLVIYQDSKR | (Human) | (Human) | (Human) | (Human) | | YYCMDV | | AVV | 2020 |
| | PSGIPERFSGSNSGNTATLTISGTQAM | | | | | | | | | (https://science.science |
| | DEADYYCQAWDSSTAVVFGGGTKLTV | | | | | | | | | mag.org/content/early/ |
| | L | | | | | | | | | 2020/06/15/science.abc |
| | | | | | | | | | | 5902) |
| CoVA2- | EIVMTQSPATLSVSPGERATLSCRASQ | IGHV3-33 | IGHJ4 | IGKV3-15 | IGKJ5 | 2766 | AKDKAPPCSS | 3968 | QQYNYWP | Philip Brouwer et al., |
| 28 | SVSSNLAWYQHKPGQAPRLLIYGASTR | (Human) | (Human) | (Human) | (Human) | | GWYYFDY | | LIT | 2020 |
| | ATGIPARFSGSGSGTEFTLTISSLQSEDF | | | | | | | | | (https://science.science |
| | AVYYCQQYNYWPLITFGQGTRLEIK | | | | | | | | | mag.org/content/early/ |
| | | | | | | | | | | 2020/06/15/science.abc |
| | | | | | | | | | | 5902) |
| CoVA2- | DIVMTQSPSSLSASVGDRVTITCRASQ | IGHV3-30 | IGHJ3 | IGKV1-39 | IGKJ1 | 2767 | ARGVEDPVV | 3969 | QQSYSTPR | Philip Brouwer et al., |
| 29 | SISSYLNWYQQKPGKAPKLLIYAASSLQ | (Human) | (Human) | (Human) | (Human) | | PAAIPWCWF | | T | 2020 |
| | SGVPSRFSGSGSGTDFTLTISSLQPEDF | | | | | | DP | | | (https://science.science |
| | ATYYCQQSYSTPRTFGQGTKVEIK | | | | | | | | | mag.org/content/early/ |
| | | | | | | | | | | 2020/06/15/science.abc |
| | | | | | | | | | | 5902) |
| CoVA2- | QSVLTQPPSVSVAPGQTARITCGGNNI | IGHV4-30- | IGHJ5 | IGLV3-21 | IGLJ3 | 2768 | ASLPVVPAAI | 3970 | QVWASSS | Philip Brouwer et al., |
| 30 | GSKSVHWYQQKPGQAPVLVVYDDSD | 4 | (Human) | (Human) | (Human) | | GPLPAFDI | | VV | 2020 |
| | RPSGIPERFSGSNSGNTATLTISRVEAG | (Human) | | | | | | | | (https://science.science |
| | DEADYYCQVWASSSVVFGGGTKLTVL | | | | | | | | | mag.org/content/early/ |
| | | | | | | | | | | 2020/06/15/science.abc |
| | | | | | | | | | | 5902) |
| CoVA2- | DIVMTQTPLSLSVTPGQPASISCKSGQ | IGHV1-2 | IGHJ3 | IGKV2D-29 | IGKJ1 | 2769 | RKMLTIFGKV | 3971 | MQSIQLPP | Philip Brouwer et al., |
| 31 | SLLHSDGKTYLYWYLQKPGQPPQLLIY | (Human) | (Human) | (Human) | (Human) | | NQTMLLISGA | | T | 2020 |
| | EVSNRFSGVPDRFSGSGSGTDFTLKISR | | | | | | KGQ | | | (https://science.science |
| | VEAEDVGVYYCMQSIQLPPTFGQGTK | | | | | | | | | mag.org/content/early/ |
| | VEIK | | | | | | | | | 2020/06/15/science.abc |
| | | | | | | | | | | 5902) |
| CoVA2- | DIVMTQSPATLSLSPGERATLSCRASQ | IGHV1-69 | IGHJ4 | IGKV3-11 | IGKJ4 | 2770 | ARTHSYDNSG | 3972 | QQRSNWP | Philip Brouwer et al., |
| 32 | SVSSFLAWYQQKPGQAPRLLIYDASNR | (Human) | (Human) | (Human) | (Human) | | QYFDY | | PRLT | 2020 |
| | ATGIPARFSGSGSGTDFTLTISSLEPEDF | | | | | | | | | (https://science.science |
| | AVYYCQQRSNWPPRLTFGGGTKVDIK | | | | | | | | | mag.org/content/early/ |
| | | | | | | | | | | 2020/06/15/science.abc |
| | | | | | | | | | | 5902) |
| CoVA2- | QSALTQPASVSGSPGQSITISCTGTSSD | IGHV5-10- | IGHJ6 | IGLV2-23 | IGLJ3 | 2771 | ARLKVITIFGV | 3973 | CSYAGSV | Philip Brouwer et al., |
| 33 | VGSYNLVSWYQQHPGKAPKLMIYDSS | 1 | (Human) | (Human) | (Human) | | VRDDYGMDV | | | 2020 |
| | KRPSGVSNRFSGSKSGNTASLTISGLQ | (Human) | | | | | | | | (https://science.science |
| | AEDEADYYCCSYAGSVFGGGTKLTVL | | | | | | | | | mag.org/content/early/ |
| | | | | | | | | | | 2020/06/15/science.abc |
| | | | | | | | | | | 5902) |
| CoVA2- | QTVVTQPASVSGSPGQSITISCTGTSSD | IGHV3-30 | IGHJ4 | IGLV2-14 | IGLJ1 | 2772 | ARSASGSYYG | 3974 | SSYTSSSTL | Philip Brouwer et al., |
| 34 | VGGYNYVSWYQQHPGKAPKLMIYDV | (Human) | (Human) | (Human) | (Human) | | AFDY | | GLYV | 2020 |
| | SNRPSGVSNRFSGSKSGNTASLTISGL | | | | | | | | | (https://science.science |
| | QAEDEADYYCSSYTSSSTLGLYVFGTGT | | | | | | | | | mag.org/content/early/ |
| | KVTVL | | | | | | | | | 2020/06/15/science.abc |
| | | | | | | | | | | 5902) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoVA2- | Ab | SARS-CoV2 | SARS- | | SARS-CoV1, | S; non- | B-cells; SARS-CoV2 | 685 | EVQLVESGAEVKKPGASVKVSCKVSGYT | 1764 |
| 37 | | | CoV1 | | SARS-CoV2 | RBD | Human Patient | | LPELSMHWVRQAPGKGLDWMGGFDP | |
| | | | | | | | | | EDGETIYAQKFQGRVTMTEDTSTDTAY | |
| | | | | | | | | | MELSSLRSEDTAVYYCATSPAVMSVGW | |
| | | | | | | | | | VDPWGQGTLVTVSS | |
| CoVA2- | Ab | SARS-CoV2 | SARS- | | SARS-CoV1, | S; non- | B-cells; SARS-CoV2 | 686 | QVQLQQSGPGLVKPSETLPLTCTVSGGS | 1765 |
| 38 | | | CoV1 | | SARS-CoV2 | RBD | Human Patient | | ISSSSYYWGWIRQPPGKGLEWIGSVFYS | |
| | | | | | | | | | GSTYYNPSLKSRVTISVDTSKNQLSLKLSS | |
| | | | | | | | | | VTAADTAVYYCARQVRQWLEDDAFDI | |
| | | | | | | | | | WGQGTMVTVSS | |
| CoVA2- | Ab | SARS-CoV2 | SARS- | | SARS-CoV1, | S; RBD | B-cells; SARS-CoV2 | 687 | QVQLVETGGGLIQPGGSLRLSCAASGFT | 1766 |
| 39 | | | CoV1 | | SARS-CoV2 | | Human Patient | | VSSNYMSWVRQAPGKGLEWVSVIYTG | |
| | | | | | | | | | GTTYYADSVKGRFTISRDNSKNTLYLQM | |
| | | | | | | | | | NSLRAEDTAVYYCARAHVDTAMVESGA | |
| | | | | | | | | | FDIWGQGTRVTVSS | |
| CoVA2- | Ab | SARS-CoV2 | SARS- | | SARS-CoV1, | S; non- | B-cells; SARS-CoV2 | 688 | QVQLQESGPGLVKPSGTLSLTCAVSGGS | 1767 |
| 40 | | | CoV1 | | SARS-CoV2 | RBD | Human Patient | | ISSSNWWSWVRQPPGKGLEWIGEIYHS | |
| | | | | | | | | | GSSNYNPSLKSRVTISVDKSKNQFSLKLN | |
| | | | | | | | | | SVTAADTAVYYCAGRYCSGGRCGWFDP | |
| | | | | | | | | | WGQGTLVTVSS | |
| CoVA2- | Ab | SARS-CoV2 | SARS- | | SARS-CoV1, | S; non- | B-cells; SARS-CoV2 | 689 | EVQLVESGGGLVKPGGSLRLSCSASGFT | 1768 |
| 41 | | | CoV1 | | SARS-CoV2 | RBD | Human Patient | | FSNYNMDWVRQAPGKGLEWVSSISSSS | |
| | | | | | | | | | SYIYLADSVKGRFTISRDNAKNSLYLQMN | |
| | | | | | | | | | SLRAEDTAVYYCARVQKDIVVVPVALAD | |
| | | | | | | | | | YYYYGMDVWGQGTTVTVSS | |
| CoVA2- | Ab | SARS-CoV2 | SARS- | | SARS-CoV1, | S; non- | B-cells; SARS-CoV2 | 690 | QVQLVQPGAEVKKPGASVKVSCKASGY | 1769 |
| 43 | | | CoV1 | | SARS-CoV2 | RBD | Human Patient | | TFTSYGISWVRQAPGQGLEWMGWISA | |
| | | | | | | | | | YNGDTNYAQKLQGRVTMTTDTSTSTAY | |
| | | | | | | | | | MELRSLKSDDTAVYYCARFDYGYPYSSW | |
| | | | | | | | | | SVLSIDYWGQGTLVTVSS | |
| CoVA2- | Ab | SARS-CoV1, | | | SARS-CoV1, | S; RBD | B-cells; SARS-CoV2 | 691 | EVQLVESGGGVVQPGRSLRLSCAASGFT | 1770 |
| 44 | | SARS-CoV2 | | | SARS-CoV2 | | Human Patient | | FSSYAMHWVRQAPGKGLEWVAVISYD | |
| | | | | | | | | | GSYKYYADSVKGRFTISRDNSKNTLYLQ | |
| | | | | | | | | | MNSLTAEDTAVYYCAREGSRQWLVIYF | |
| | | | | | | | | | DYWGQGTLVTVSS | |
| CoVA2- | Ab | SARS-CoV2 | | | SARS-CoV1, | S; RBD | B-cells; SARS-CoV2 | 692 | EVQLLESGAEVKKPGASVKISCKASGYTF | 1771 |
| 45 | | | | | SARS-CoV2 | | Human Patient | | TGYYMHWVRQAPGQGLEWMGWINP | |
| | | | | | | | | | NSGGTNYAQKFQGRVTMTRDTSISTAY | |
| | | | | | | | | | MELSRLRSDDTAVYYCARGDGDYYDSS | |
| | | | | | | | | | GYYRPTLYNWLDPWGQGTRVTVSS | |
| CoVA2- | Ab | SARS-CoV2 | SARS- | | SARS-CoV1, | S; RBD | B-cells; SARS-CoV2 | 693 | QVQLQESGPGLVKPSETLSLTCTASGGSI | 1772 |
| 46 | | | CoV1 | | SARS-CoV2 | | Human Patient | | SSSSYYWGWIRQPPGKGLEWIGSIYYSG | |
| | | | | | | | | | STYYNPSLKSRVTISVDTSKNQFSLKLSSV | |
| | | | | | | | | | TAADTAVYYCARHPSGLYQLLNWGQGT | |
| | | | | | | | | | LVTVSS | |
| CoVA2- | Ab | SARS-CoV2 | SARS- | | SARS-CoV1, | S; non- | B-cells; SARS-CoV2 | 694 | EVQLVESGGGLVQPGRSLRLSCAASGFT | 1773 |
| 47 | | | CoV1 | | SARS-CoV2 | RBD | Human Patient | | FDDYAMHWVRQAPGKGLEWVSGISW | |
| | | | | | | | | | NSGSIGYADSVKGRFTISRDSAKNSLYLQ | |
| | | | | | | | | | MNSLRAEDTALYYCAKVATYYYDRSGYY | |
| | | | | | | | | | YGGALDYWGQGTLVTVSS | |
| CoVA3- | Ab | SARS-CoV1, | | | SARS-CoV1, | S; non- | B-cells; SARS-CoV2 | 695 | EVQLVESGPGLVKPSETLSLTCTVSGGSI | 1774 |
| 01 | | SARS-CoV2 | | | SARS-CoV2 | RBC | Human Patient | | SSYYWSWIRQPPGKGLEWIGYIYYSGST | |
| | | | | | | | | | NYNPSLKSRVTISVDTSKNQFSLKLSSVTA | |
| | | | | | | | | | ADTAVYYCARGPAATYYYYMDVWGKG | |
| | | | | | | | | | TRVTVSS | |
| CoVA3- | Ab | SARS-CoV1, | | | SARS-CoV1, | S; non- | B-cells; SARS-CoV2 | 696 | EVQLVESGGGLVQPGGSLRLSCAASGFT | 1775 |
| 03 | | SARS-CoV2 | | | SARS-CoV2 | RBD | Human Patient | | FSSYAMSWVRQAPGKGLEWVSTISGSG | |
| | | | | | | | | | GNTYYADSVKGRFTISRDNSKNTLYLQM | |
| | | | | | | | | | NSLRAEDTAVYYCAKEIAVAGCFDYWG | |
| | | | | | | | | | QGTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoVA2- | QSVLTQPPSVSGAPGQRVTISCTGSSS | IGHV1-24 | IGHJ5 | IGLV1-40 | IGLJ3 | 2773 | ATSPAVMSV | 3975 | QSYDSSLS | Philip Brouwer et al., |
| 37 | NIGAGYDVHWYQQLPGTAPKVLIYDN | (Human) | (Human) | (Human) | (Human) | | GWVDP | | GSV | 2020 |
| | NNRPSGVPDRFSGSKSGTSASLAITGL | | | | | | | | | (https://science.science |
| | QAEDEADYYCQSYDSSLSGSVFGGGT | | | | | | | | | mag.org/content/early/ |
| | KLTVL | | | | | | | | | 2020/06/15/science.abc |
| | | | | | | | | | | 5902) |
| CoVA2- | DIQLTQSPSSLSASVGDRVTITCRASQG | IGHV4-39 | IGHJ3 | IGKV1-17 | IGKJ4 | 2774 | ARQVRQWLE | 3976 | LQHNSYPL | Philip Brouwer et al., |
| 38 | IRNDLGWYQQKPGKAPKRLIYAASSL | (Human) | (Human) | (Human) | (Human) | | DDAFDI | | T | 2020 |
| | QSGVPSRFSGSGSGTEFTLTISSLQPED | | | | | | | | | (https://science.science |
| | FATYYCLQHNSYPLTFGGGTKVDIK | | | | | | | | | mag.org/content/early/ |
| | | | | | | | | | | 2020/06/15/science.abc |
| | | | | | | | | | | 5902) |
| CoVA2- | QSALTQPASVSGSPGQSITISCTGTSSD | IGHV3-53 | IGHJ3 | IGLV2-23 | IGLJ3 | 2775 | ARAHVDTAM | 3977 | CSYAGSST | Philip Brouwer et al., |
| 39 | VGSYNLVSWYQQHPGKAPKLMIYEVT | (Human) | (Human) | (Human) | (Human) | | VESGAFDI | | WV | 2020 |
| | KRPSGVSNRFSGSKSGNTASLTISGLQ | | | | | | | | | (https://science.science |
| | AEDEADYYCCSYAGSSTWVFGGGTKL | | | | | | | | | mag.org/content/early/ |
| | TVL | | | | | | | | | 2020/06/15/science.abc |
| | | | | | | | | | | 5902); Nicholas Wu |
| | | | | | | | | | | (https://www.biorxiv.or |
| | | | | | | | | | | g/content/10.1101/202 |
| | | | | | | | | | | 0.07.26.222232v1) |
| CoVA2- | QSVLTQPASVSGSPGQSITISCTGTSSD | IGHV4-4 | IGHJ5 | IGLV2-23 | IGLJ3 | 2776 | AGRYCSGGRC | 3978 | CSYAGSST | Philip Brouwer et al., |
| 40 | VGSYNLVSWYQQHPGKAPKLMIYEAS | (Human) | (Human) | (Human) | (Human) | | GWFDP | | WV | 2020 |
| | KRPSGISNRFSGSKSGNTASLTISGLQA | | | | | | | | | (https://science.science |
| | EDEADYYCCSYAGSSTWVFGGGTKPT | | | | | | | | | mag.org/content/early/ |
| | VL | | | | | | | | | 2020/06/15/science.abc |
| | | | | | | | | | | 5902) |
| CoVA2- | DIVMTQSPATLSVSPGERATLSCRASQ | IGHV3-21 | IGHJ6 | IGKV3-15 | IGKJ1 | 2777 | ARVQKDIVVV | 3979 | QQCYNWP | Philip Brouwer et al., |
| 41 | SVSSNLAWYQQKPGQAPRLLIYGAST | (Human) | (Human) | (Human) | (Human) | | PVALADYYYY | | PWT | 2020 |
| | RATGIPARFSGSGSGTEFTLTISSLQPE | | | | | | GMDV | | | (https://science.science |
| | DFAVYYCQQCYNWPPWTFGQGTRVE | | | | | | | | | mag.org/content/early/ |
| | FK | | | | | | | | | 2020/06/15/science.abc |
| | | | | | | | | | | 5902) |
| CoVA2- | DIVMTQSPSSLSASVGDRVTITCRASQ | IGHV1-18 | IGHJ4 | IGKV1-17 | IGKJ4 | 2778 | ARFDYGYPYS | 3980 | LQHNSYPL | Philip Brouwer et al., |
| 43 | GIRNDLGWYQQKPGKAPKRLIYAASSL | (Human) | (Human) | (Human) | (Human) | | SWSVLSIDY | | T | 2020 |
| | QSGVPSRFSGSGSGTEFTLTISSLQPED | | | | | | | | | (https://science.science |
| | FATYYCLQHNSYPLTFGGGTKLEIK | | | | | | | | | mag.org/content/early/ |
| | | | | | | | | | | 2020/06/15/science.abc |
| | | | | | | | | | | 5902) |
| CoVA2- | AIRMTQSPSSLSASVGDRVTITCRASQ | IGHV3-30 | IGHJ4 | IGKV1-39 | IGKJ2 | 2779 | AREGSRQWL | 3981 | QQSYTTFIY | Philip Brouwer et al., |
| 44 | SISSYLNWYQQKPGKAPKLLIYAASSLQ | (Human) | (Human) | (Human) | (Human) | | VIYFDY | | T | 2020 |
| | SGVPSRFSGSGSGTDFTLTISSLQPEDF | | | | | | | | | (https://science.science |
| | ATYYCQQSYTTFIYTFGQGTKLEIK | | | | | | | | | mag.org/content/early/ |
| | | | | | | | | | | 2020/06/15/science.abc |
| | | | | | | | | | | 5902) |
| CoVA2- | DIVMTQSPGTLSLSPGERATLSCRASQ | IGHV1-2 | IGHJ5 | IGKV3-20 | IGKJ1 | 2780 | ARGDGDYYD | 3982 | QQYGSSPY | Philip Brouwer et al., |
| 45 | SVSSSYLAWYQQKPGQAPRLLIYGASS | (Human) | (Human) | (Human) | (Human) | | SSGYYRPTLY | | T | 2020 |
| | RATGIPDRFSGSGSGTDFTLTISRLEPE | | | | | | NWLDP | | | (https://science.science |
| | DFAVYYCQQYGSSPYTFGQGTKVEIK | | | | | | | | | mag.org/content/early/ |
| | | | | | | | | | | 2020/06/15/science.abc |
| | | | | | | | | | | 5902) |
| CoVA2- | AIRMTQSPSSLSASVGDRVTITCQASQ | IGHV4-39 | IGHJ4 | IGKV1-33 | IGKJ4 | 2781 | ARHPSGLYQL | 3983 | QQYDNLLS | Philip Brouwer et al., |
| 46 | DISNYLNWYQQKPGKAPKLLIYDASNL | (Human) | (Human) | (Human) | (Human) | | LN | | LT | 2020 |
| | ETGVPSRFSGSGSGTDFTFTISSLQPED | | | | | | | | | (https://science.science |
| | IATYYCQQYDNLLSLTFGGGTKVEIK | | | | | | | | | mag.org/content/early/ |
| | | | | | | | | | | 2020/06/15/science.abc |
| | | | | | | | | | | 5902) |
| CoVA2- | EIVLTQSPDSLAVSLGERATINCKSSQS | IGHV3-9 | IGHJ4 | IGKV4-1 | IGKJ4 | 2782 | AKVATYYYDR | 3984 | QQYYSTPP | Philip Brouwer et al., |
| 47 | VLYSSNNKNYLAWYQQKPGQPPKLLI | (Human) | (Human) | (Human) | (Human) | | SGYYYGGALD | | LT | 2020 |
| | YWASTRESGVPDRFSGSGSGTHFTLTI | | | | | | Y | | | (https://science.science |
| | SSLQAEDVAVYYCQQYYSTPPLTFGGG | | | | | | | | | mag.org/content/early/ |
| | TKVDIK | | | | | | | | | 2020/06/15/science.abc |
| | | | | | | | | | | 5902) |
| CoVA3- | EIVMTQSPSSLSASVGDRVTITCRASQS | IGHV4-59 | IGHJ6 | IGKV1-39 | IGKJ4 | 2783 | ARGPAATYYY | 3985 | QQSYSTLT | Philip Brouwer et al., |
| 01 | MSSYLNWYQQKPGKAPKLLIYAASSL | (Human) | (Human) | (Human) | (Human) | | YMDV | | | 2020 |
| | QSGVPSRFSGSGSGTDFTLTISSLQPED | | | | | | | | | (https://science.science |
| | FATYYCQQSYSTLTFGGGTKVEIK | | | | | | | | | mag.org/content/early/ |
| | | | | | | | | | | 2020/06/15/science.abc |
| | | | | | | | | | | 5902) |
| CoVA3- | QTVVTQPPSVSVSPGQTARITCSGDAL | IGHV3-23 | IGHJ4 | IGLV3-25 | IGLJ3 | 2784 | AKEIAVAGCF | 3986 | QSADSSGT | Philip Brouwer et al., |
| 03 | PKQYAYWYQQKPGQAPVLVIYKDSER | (Human) | (Human) | (Human) | (Human) | | DY | | YRV | 2020 |
| | PSGIPERFSGSSSGTTVTLTISGVQAED | | | | | | | | | (https://science.science |
| | EADYYCQSADSSGTYRVFGGGTKLTVL | | | | | | | | | mag.org/content/early/ |
| | | | | | | | | | | 2020/06/15/science.abc |
| | | | | | | | | | | 5902) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CoVA3- | Ab | SARS-CoV2 | SARS- | | SARS-CoV1, | S; non- | B-cells; SARS-CoV2 | 697 | EVQLVESGGGVVQPGRSLRLSCAASGFT | 1776 |
| 04 | | | CoV1 | | SARS-CoV2 | RBD | Human Patient | | FSSYGMHWVRQAPGKGLEWVAVIWY | |
| | | | | | | | | | DGSNKYYADSVKGRFTISRDNSKNTLYL | |
| | | | | | | | | | QMNSLRAEDTAVYYCARVGSVKSTAGY | |
| | | | | | | | | | DFWSGDPFDYWGQGTLVTVSS | |
| CoVA3- | Ab | SARS-CoV2 | SARS- | | SARS-CoV1, | S; RBD | B-cells; SARS-CoV2 | 698 | QVQLVQSGAEVKKPGASVKVSCKVSGY | 1777 |
| 05 | | | CoV1 | | SARS-CoV2 | | Human Patient | | TLTELSMHWVRQAPGKGLEWMGGFD | |
| | | | | | | | | | PEDGETIYAQKFQGRVTMTEDTSTDTAY | |
| | | | | | | | | | MELSSLRSEDTAVYYCATAYSVDTAMVR | |
| | | | | | | | | | GVGYWGQGTLVTVSS | |
| CoVA3- | Ab | SARS-CoV2 | SARS- | | SARS-CoV1, | S; RBD | B-cells; SARS-CoV2 | 699 | QVQLVESRAEVKKPGSSVKVSCKASGGT | 1778 |
| 06 | | | CoV1 | | SARS-CoV2 | | Human Patient | | FSSYAISWVRQAPGQGLEWMGRIIPILG | |
| | | | | | | | | | ITNYAQKFQGRVTITADKSTSTAYMELSS | |
| | | | | | | | | | LRSEDTAVYYCARDAPDYYDSSGPTYFD | |
| | | | | | | | | | YWGQGTLVTVSS | |
| CoVA3 | Ab | SARS-CoV2 | SARS- | | SARS-CoV1, | S; non- | B-cells; SARS-CoV2 | 700 | QVQLVESGGGLVQPGRSLRLSCAASGFT | 1779 |
| 07 | | | CoV1 | | SARS-CoV2 | RBD | Human Patient | | FDDYAMHWVRQAPGKGLEWVSGISW | |
| | | | | | | | | | NSGSIGYADSVKGRFTISRDNAKNSLYLQ | |
| | | | | | | | | | MNSLRAEDTALYYCAKAEPEVGGYDYY | |
| | | | | | | | | | MDVWGKGTMVTVSS | |
| CoVA3- | Ab | SARS-CoV1, | | | SARS-CoV1, | S; non- | B-cells; SARS-CoV2 | 701 | EVQLVESGPGLVKPSETLSLTCTVSGGSI | 1780 |
| 08 | | SARS-CoV2 | | | SARS-CoV2 | RBD | Human Patient | | SSYYWSWIRQPPGKGLEWIGYIYYSGST | |
| | | | | | | | | | NYNPSLKSRVTISVDTSKNQFSLRLSSVT | |
| | | | | | | | | | AADTAVYYCARGPAATYYYYMDVWGK | |
| | | | | | | | | | GTMVTVSS | |
| CoVA3- | Ab | SARS-CoV2 | SARS- | | SARS-CoV1, | S; RBD | B-cells; SARS-CoV2 | 702 | EVQLVESGGGLVQPGRSLRLSCAASGFT | 1781 |
| 09 | | | CoV1 | | SARS-CoV2 | | Human Patient | | FDDYAMHWVRQAPGKGLEWVSGISW | |
| | | | | | | | | | NSGSIGYADSVKGRFTISRDNAKNSLYLQ | |
| | | | | | | | | | MNSLRAEDTALYYCAKMGPDPAHDYG | |
| | | | | | | | | | RKNDAFDIWGQGTMVTVSS | |
| CoVA3- | Ab | SARS-CoV1, | | | SARS-CoV1, | S; RBD | B-cells; SARS-CoV2 | 703 | EVQLVESRAEMKKPGESLKISCKGSGYTF | 1782 |
| 10 | | SARS-CoV2 | | | SARS-CoV2 | | Human Patient | | TNHWIAWVRQMPGKGLEWMGIIYPG | |
| | | | | | | | | | DSDTRYSPSFEGQVTISADKSISTAYLQW | |
| | | | | | | | | | SSLKASDTAMYYCARRGYTYGADFYGLD | |
| | | | | | | | | | VWGQGTRVTVSS | |
| CR3022 | Ab | SARS-CoV1, | | SARS-CoV1 | SARS-CoV2 | S; RBD | B-cells; SARS-CoV1 | 704 | QMQLVQSGTEVKKPGESLKISCKGSGY | 1783 |
| | | SARS-CoV2 | | | | | Human Patient | | GFITYWIGWVRQMPGKGLEWMGIIYP | |
| | | | | | | | | | GDSETRYSPSFQGQVTISADKSINTAYLQ | |
| | | | | | | | | | WSSLKASDTAIYYCAGGSGISTPMDVW | |
| | | | | | | | | | GQGTTVTVSS | |
| CV-X1- | Ab | SARS-CoV2 | SARS- | SARS-CoV2 | | S; RBD | B-cells; SARS-CoV2 | | ND | |
| 126 | | | CoV1 | | | | Human Patient | | | |
| CV-X2- | Ab | SARS-CoV2 | SARS- | SARS-CoV2 | | S; RBD | B-cells; SARS-CoV2 | | ND | |
| 106 | | | CoV1 | | | | Human Patient | | | |
| CV05-163 | Ab | SARS-CoV2 | SARS- | SARS-CoV2 | | S; RBD | B-cells; SARS-CoV2 | | ND | |
| | | | CoV1 | | | | Human Patient | | | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CoVA3- | QPVLTQPPSVSVAPGQTARITCGGNN | IGHV3-33 | IGHJ4 | IGLV3-21 | IGLJ1 | 2785 | ARVGSVKSTA | 3987 | QVWDSSS | Philip Brouwer et al. |
| 04 | IGSKSVHWYQQKPGQAPVLVVYDDS | (Human) | (Human) | (Human) | (Human) | | GYDFWSGDP | | DHYV | 2020 |
| | DRPSGIPERFSGSNSGNTATLTISRVEA | | | | | | FDY | | | (https://science.science |
| | GDEADYYCQVWDSSSDHYVFGTGTK | | | | | | | | | mag.org/content/early/ |
| | VTVL | | | | | | | | | 2020/06/15/science.abc |
| | | | | | | | | | | 5902) |
| CoVA3- | NFMLTQPPSASGTPGQRVTISCSGSSS | IGHV1-24 | IGHJ4 | IGLV1-44 | IGLJ3 | 2786 | ATAYSVDTA | 3988 | AAWDDSL | Philip Brouwer et al., |
| 05 | NIGSNTVNWYQQLPGTAPKLLIYSNN | (Human) | (Human) | (Human) | (Human) | | MVRGVGY | | NGPHWV | 2020 |
| | QRPSGVPDRFSGSKSGTSASLAISGLQ | | | | | | | | | (https://science.science |
| | SEDEADYYCAAWDDSLNGPHWVFGG | | | | | | | | | mag.org/content/early/ |
| | GTKLTVL | | | | | | | | | 2020/06/15/science.abc |
| | | | | | | | | | | 5902) |
| CoVA3- | DVVMTQSPDSLAVSLGERATINCKSS | IGHV1-69 | IGHJ4 | IGKV4-1 | IGKJ4 | 2787 | ARDAPDYYDS | 3989 | QQYYSTPL | Philip Brouwer et al., |
| 06 | QSVLYSSNNKNYLAWYQQKPGQPPKL | (Human) | (Human) | (Human) | (Human) | | SGPTYFDY | | T | 2020 |
| | LIYWASTRESGVPDRFSGSGSGTDFTL | | | | | | | | | (https://science.science |
| | TISSLQAEDVAVYYCQQYYSTPLTFGG | | | | | | | | | mag.org/content/early/ |
| | GTKVDIK | | | | | | | | | 2020/06/15/science.abc |
| | | | | | | | | | | 5902) |
| CoVA3 | EIVLTQSPGTLSLSPGERATLSCRASQS | IGHV3-9 | IGHJ6 | IGKV3-20 | IGKJ3 | 2788 | AKMGPDPAH | 3990 | QQYGSSPF | Philip Brouwer et al., |
| 07 | VSSSYLAWYQQKPGQAPRLLIYGASSR | (Human) | (Human) | (Human) | (Human) | | DYGRKNDAF | | T | 2020 |
| | ATGIPDRFSGSGSGTDFTLTISRLEPED | | | | | | DI | | | (https://science.science |
| | FAVYYCQQYGSSPFTFGPGTKLEIK | | | | | | | | | mag.org/content/early/ |
| | | | | | | | | | | 2020/06/15/science.abc |
| | | | | | | | | | | 5902) |
| CoVA3- | DIVMTQSPSSLSASVGDRVTITCRASQ | IGHV4-59 | IGHJ6 | IGKV1-39 | IGKJ4 | 2789 | AKAEPEVGGY | 3991 | QQSYSTPL | Philip Brouwer et al., |
| 08 | SISSYLNWYQQKPGKAPKLLIYAASSLQ | (Human) | (Human) | (Human) | (Human) | | DYYMDV | | T | 2020 |
| | SGVPSRFSGSGSGTDFTLTISSLQPEDF | | | | | | | | | (https://science.science |
| | ATYYCQQSYSTPLTFGPGTKLEIK | | | | | | | | | mag.org/content/early/ |
| | | | | | | | | | | 2020/06/15/science.abc |
| | | | | | | | | | | 5902) |
| CoVA3- | DIVMTQSPSSLSASVGDRVTITCRASQ | IGHV3-9 | IGHJ3 | IGKV1-39 | IGKJ4 | 2790 | ARGPAATYYY | 3992 | QQSYSTLT | Philip Brouwer et al., |
| 09 | SMSSYLNWYQQKPGKAPKLLIYAASSL | (Human) | (Human) | (Human) | (Human) | | YMDV | | | 2020 |
| | QSGVPSRFSGSGSGTDFTLTISSLQPED | | | | | | | | | (https://science.science |
| | FATYYCQQSYSTLTFGGGTKLEIK | | | | | | | | | mag.org/content/early/ |
| | | | | | | | | | | 2020/06/15/science.abc |
| | | | | | | | | | | 5902) |
| CoVA3- | DIVMTQSPDSLAVSLGERATINCKSSQ | IGHV5-51 | IGHJ6 | IGKV4-1 | IGKJ5 | 2791 | ARRGYTYGAD | 3993 | QQYYSTP | Philip Brouwer et al., |
| 10 | SVLYSSNNKNYLAWYQQKPGQPPKLLI | (Human) | (Human) | (Human) | (Human) | | FYGLDV | | IT | 2020 |
| | YWASTRESGVPDRFSGSGSGTDFTLTI | | | | | | | | | (https://science.science |
| | SSLQAEDVAVYYCQQYYSTPITFGQGT | | | | | | | | | mag.org/content/early/ |
| | RLEIK | | | | | | | | | 2020/06/15/science.abc |
| | | | | | | | | | | 5902) |
| CR3022 | DIQLTQSPDSLAVSLGERATINCKSSQS | IGHV5-51 | IGHJ6 | IGKV4-1 | IGKJ1 | 2792 | AGGSGISTPM | 3994 | QQYYSTPY | Jan ter Meulen et al., |
| | VLYSSINKNYLAWYQQKPGQPPKLLIY | (Human) | (Human) | (Human) | (Human) | | DV | | T | 2006 |
| | WASTRESGVPDRFSGSGSGTDFTLTIS | | | | | | | | | (https://journals.plos.or |
| | SLQAEDVAVYYCQQYYSTPYTFGQGT | | | | | | | | | g/plosmedicine/article?i |
| | KVEIK | | | | | | | | | d=10.1371/journal.pme |
| | | | | | | | | | | d.0030237); Meng Yuan |
| | | | | | | | | | | et al., 2020a |
| | | | | | | | | | | (https://science.science |
| | | | | | | | | | | mag.org/content/early/ |
| | | | | | | | | | | 2020/04/02/science.abb |
| | | | | | | | | | | 7269); Meng Yuan et al., |
| | | | | | | | | | | 2020b |
| | | | | | | | | | | (https://www.biorxiv.or |
| | | | | | | | | | | g/content/10.1101/202 |
| | | | | | | | | | | 0.06.08.141267v1) |
| CV-X1- | ND | IGHV3-53 | IGHJ6 | ND | ND | 2793 | ARDLSEGGM | 3995 | QQANGFP | Jakob Kreye et al., 2020 |
| 126 | | (Human) | (Human) | | | | DV | | PL | (https://www.biorxiv.or |
| | | | | | | | | | | g/content/10.1101/202 |
| | | | | | | | | | | 0.08.15.252320v1.full.p |
| | | | | | | | | | | df) |
| CV-X2- | ND | IGHV1-69 | IGHJ6 | ND | ND | 2794 | ATRKETTVTTS | 3996 | QQSYSTPY | Jakob Kreye et al., 2020 |
| 106 | | (Human) | (Human) | | | | LVYGMDV | | T | (https://www.biorxiv.or |
| | | | | | | | | | | g/content/10.1101/202 |
| | | | | | | | | | | 0.08.15.252320v1.full.p |
| | | | | | | | | | | df) |
| CV05-163 | ND | IGHV1-2 | IGHJ6 | ND | ND | 2795 | AREVMVRGA | 3997 | QQRSNWP | Jakob Kreye et al., 2020 |
| | | (Human) | (Human) | | | | LPPYGMDV | | PVT | (https://www.biorxiv.or |
| | | | | | | | | | | g/content/10.1101/202 |
| | | | | | | | | | | 0.08.15.252320v1.full.p |
| | | | | | | | | | | df) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CV07-200 | Ab | SARS-CoV2 | SARS- | SARS-CoV2 | | S; RBD | B-cells; SARS-CoV2 | | ND | |
| | | | CoV1 | | | | Human Patient | | | |
| CV07-209 | Ab | SARS-CoV2 | SARS- | SARS-CoV2 | | S; RBD | B-cells; SARS-CoV2 | | ND | |
| | | | CoV1 | | | | Human Patient | | | |
| CV07-222 | Ab | SARS-CoV2 | SARS- | SARS-CoV2 | | S; RBD | B-cells; SARS-CoV2 | | ND | |
| | | | CoV1 | | | | Human Patient | | | |
| CV07-250 | Ab | SARS-CoV2 | SARS- | SARS-CoV2 | | S; RBD | B-cells; SARS-CoV2 | | ND | |
| | | | CoV1 | | | | Human Patient | | | |
| CV07-255 | Ab | SARS-CoV2 | SARS- | SARS-CoV2 | | S; RBD | B-cells; SARS-CoV2 | | ND | |
| | | | CoV1 | | | | Human Patient | | | |
| CV07-262 | Ab | SARS-CoV2 | SARS- | SARS-CoV2 | | S; RBD | B-cells; SARS-CoV2 | | ND | |
| | | | CoV1 | | | | Human Patient | | | |
| CV07-270 | Ab | SARS-CoV2 | SARS- | SARS-CoV2 | | S; RBD | B-cells; SARS-CoV2 | | ND | |
| | | | CoV1 | | | | Human Patient | | | |
| CV07-283 | Ab | SARS-CoV2 | SARS- | SARS-CoV2 | | S; RBD | B-cells; SARS-CoV2 | | ND | |
| | | | CoV1 | | | | Human Patient | | | |
| CV07-287 | Ab | SARS-CoV2 | SARS- | SARS-CoV2 | | S; RBD | B-cells; SARS-CoV2 | | ND | |
| | | | CoV1 | | | | Human Patient | | | |
| CV07-315 | Ab | SARS-CoV2 | SARS- | SARS-CoV2 | | S; RBD | B-cells; SARS-CoV2 | | ND | |
| | | | CoV1 | | | | Human Patient | | | |
| CV1 | Ab | SARS-CoV1, | | SARS-CoV2 | | S; non- | B-cells; SARS-CoV2 | 705 | QVQLQESGPGLVKPSETLSLTCTVSGYSI | 1784 |
| | | SARS-CoV2 | | | | RBD | Human Patient | | SSGYYWGWIRQPPGKGLEWIGSIYHSG | |
| | | | | | | | | | STYYNPSLKSRVTISVDTSKNQFSLKLSSV | |
| | | | | | | | | | TAADTAVYYCARTPLSLRLRYNWYFDLW | |
| | | | | | | | | | GRGTLVTVSS | |
| CV10 | Ab | SARS-CoV1, | | | SARS-CoV2 | S; non- | B-cells; SARS-CoV2 | 706 | QVQLQESGPGLVKPSETLSLTCNVSGGSI | 1785 |
| | | SARS-CoV2 | | | | RBD | Human Patient | | SSYYWSWIRQPPGKGLEWIGYIYYSGST | |
| | | | | | | | | | NYNPSLKSRVTISVDTSKNQFSLKLSSVTA | |
| | | | | | | | | | ADTAVYYCARGFDYWGQGTLVTVSS | |
| CV11 | Ab | SARS-CoV1, | | | SARS-CoV2 | S; non- | B-cells; SARS-CoV2 | 707 | QVQLQESGPGLVKPSQTLSLTCTVSGGSI | 1786 |
| | | SARS-CoV2 | | | | RBD | Human Patient | | SSGGYYWSWIRQHPGKGLEWIGYIYYS | |
| | | | | | | | | | GSTYYNPSLKSRVTISVDTSKNQFSLKLSS | |
| | | | | | | | | | VTAADTAVYYCARETTGHFDYWGQGTL | |
| | | | | | | | | | VTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CV07-200 | ND | IGHV1-2 | IGHJ6 | ND | ND | 2796 | ARGPFYYDNS | 3998 | SSYTSSSTY | Jakob Kreye et al., 2020 |
| | | (Human) | (Human) | | | | GTLGGLDV | | V | (https://www.biorxiv.or |
| | | | | | | | | | | g/content/10.1101/202 |
| | | | | | | | | | | 0.08.15.252320v1.full.p |
| | | | | | | | | | | df) |
| CV07-209 | ND | IGHV3-11 | IGHJ4 | ND | ND | 2797 | ARDGVIPPRF | 3999 | QQYDNLPL | Jakob Kreye et al., 2020 |
| | | (Human) | (Human) | | | | DY | | T | (https://www.biorxiv.or |
| | | | | | | | | | | g/content/10.1101/202 |
| | | | | | | | | | | 0.08.15.252320v1.full.p |
| | | | | | | | | | | df) |
| CV07-222 | ND | IGHV1-2 | IGHJ3 | ND | ND | 2798 | ARGPYYYDSS | 4000 | CSYAGGST | Jakob Kreye et al., 2020 |
| | | (Human) | (Human) | | | | GSLGAFDI | | SYV | (https://www.biorxiv.or |
| | | | | | | | | | | g/content/10.1101/202 |
| | | | | | | | | | | 0.08.15.252320v1.full.p |
| | | | | | | | | | | df) |
| CV07-250 | ND | IGHV1-18 | IGHJ6 | ND | ND | 2799 | AGSDNYGFPY | 4001 | SSYAGNND | Jakob Kreye et al., 2020 |
| | | (Human) | (Human) | | | | NGMDV | | FV | (https://www.biorxiv.or |
| | | | | | | | | | | g/content/10.1101/202 |
| | | | | | | | | | | 0.08.15.252320v1.full.p |
| | | | | | | | | | | df) |
| CV07-255 | ND | IGHV1-2 | IGHJ4 | ND | ND | 2800 | ARDSRFSYVN | 4002 | CSYAGHST | Jakob Kreye et al., 2020 |
| | | (Human) | (Human) | | | | GEFDY | | WV | (https://www.biorxiv.or |
| | | | | | | | | | | g/content/10.1101/202 |
| | | | | | | | | | | 0.08.15.252320v1.full.p |
| | | | | | | | | | | df) |
| CV07-262 | ND | IGHV1-2 | IGHJ6 | ND | ND | 2801 | ARVGWYDFG | 4003 | CSYAGTST | Jakob Kreye et al., 2020 |
| | | (Human) | (Human) | | | | TPGDYYYYYG | | FV | (https://www.biorxiv.or |
| | | | | | | | MDV | | | g/content/10.1101/202 |
| | | | | | | | | | | 0.08.15.252320v1.full.p |
| | | | | | | | | | | df) |
| CV07-270 | ND | IGHV1-2 | IGHJ6 | ND | ND | 2802 | ARVFGPGLDC | 4004 | CSYAGSSS | Jakob Kreye et al., 2020 |
| | | (Human) | (Human) | | | | SSTSCYTYGM | | WV | (https://www.biorxiv.or |
| | | | | | | | DV | | | g/content/10.1101/202 |
| | | | | | | | | | | 0.08.15.252320v1.full.p |
| | | | | | | | | | | df) |
| CV07-283 | ND | IGHV1-2 | IGHJ6 | ND | ND | 2803 | VRGPFYYDSS | 4005 | SSYTSSSTY | Jakob Kreye et al., 2020 |
| | | (Human) | (Human) | | | | GPLGGMDV | | V | (https://www.biorxiv.or |
| | | | | | | | | | | g/content/10.1101/202 |
| | | | | | | | | | | 0.08.15.252320v1.full.p |
| | | | | | | | | | | df) |
| CV07-287 | ND | IGHV1-58 | IGHJ3 | ND | ND | 2804 | AAPYCSSTNC | 4006 | QQYYGSSP | Jakob Kreye et al., 2020 |
| | | (Human) | (Human) | | | | YDAFDI | | WT | (https://www.biorxiv.or |
| | | | | | | | | | | g/content/10.1101/202 |
| | | | | | | | | | | 0.08.15.252320v1.full.p |
| | | | | | | | | | | df) |
| CV07-315 | ND | IGHV3-9 | IGHJ6 | ND | ND | 2805 | AKDFLWDLH | 4007 | QQSYSTHA | Jakob Kreye et al., 2020 |
| | | (Human) | (Human) | | | | PPRYYGMDV | | | (https://www.biorxiv.or |
| | | | | | | | | | | g/content/10.1101/202 |
| | | | | | | | | | | 0.08.15.252320v1.full.p |
| | | | | | | | | | | df) |
| CV1 | QSVLTQPPSASGTPGQRVTISCSGSSS | IGHV4-38- | IGHJ2 | IGLV1-44 | IGLJ3 | 2806 | ARTPLSLRLRY | 4008 | AAWDDSL | Emilie Seydoux et al., |
| | NIGSNTVNWYQQLPGTAPKLLIYSNN | 2 | (Human) | (Human) | (Human) | | NWYFDL | | NGPV | 2020 |
| | QRPSGVPDRFSGSKSGTSASLAISGLQ | (Human) | | | | | | | | (https://www.biorxiv.or |
| | SEDEADYYCAAWDDSLNGPVFGGGT | | | | | | | | | g/content/10.1101/202 |
| | KLTVL | | | | | | | | | 0.05.12.091298v1) |
| CV10 | EIVLTQSPGTLSLSPGERATLSCRASQS | IGHV4-59 | IGHJ4 | IGKV3-20 | IGKJ1 | 2807 | ARGFDY | 4009 | QQYAGSP | Emilie Seydoux et al., |
| | VSSIYLAWYQQKPGQAPRLLIYGASSR | (Human) | (Human) | (Human) | (Human) | | | | WT | 2020 |
| | ATGIPDRFSGSGSGTDFTLTISRLEPED | | | | | | | | | (https://www.biorxiv.or |
| | FAVYYCQQYAGSPWTFGQGTKVEIK | | | | | | | | | g/content/10.1101/202 |
| | | | | | | | | | | 0.05.12.091298v1) |
| CV11 | EIVLTQSPATLSLSPGERATLSCRASQS | IGHV4-31 | IGHJ4 | IGKV3-11 | IGKJ3 | 2808 | ARETTGHFDY | 4010 | PIFT | Emilie Seydoux et al., |
| | VSSYLAWYQQKPGQAPRLLIYDASNR | (Human) | (Human) | (Human) | (Human) | | | | QQRSNWP | 2020 |
| | ATGIPARFSGSGSGTDFTLTISSLEPEDF | | | | | | | | | (https://www.biorxiv.or |
| | AVYYCQQRSNWPPIFTFGPGTKVDIK | | | | | | | | | g/content/10.1101/202 |
| | | | | | | | | | | 0.05.12.091298v1) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CV12 | Ab | SARS-CoV2 | SARS- | | SARS-CoV2 | S; non- | B-cells; SARS-CoV2 | 708 | QVQLVESGGGVVQPGRSLRLSCAASGF | 1787 |
| | | | CoV1 | | | RBD | Human Patient | | NLSNYAMHWVRQASGKGLEWVSLISY | |
| | | | | | | | | | DGSVKYYTDSVKGRFTVSGDNSKNTLFL | |
| | | | | | | | | | QMNSLRPDDSALYYCVRGGVSGPNSFD | |
| | | | | | | | | | MWGQGTTVTVSS | |
| CV13 | Ab | SARS-CoV1, | | | SARS-CoV2 | S; non- | B-cells; SARS-CoV2 | 709 | QVQLVQSGSELKKPGASVKLSCQASGYS | 1788 |
| | | SARS-CoV2 | | | | RBD | Human Patient | | FTNHAMNWVRQAPGQGLEWMGWIN | |
| | | | | | | | | | TNTGNPTYAQGFTGRFVFSLDTSVSTTYL | |
| | | | | | | | | | HISSLKAEDTAVYYCARASARPGVATNL | |
| | | | | | | | | | DFWGQGTLVVVSS | |
| CV15 | Ab | SARS-CoV2 | SARS- | | SARS-CoV2 | S; non- | B-cells; SARS-CoV2 | 710 | EVQLVESGGGLVQPGGSLRLSCAASGFT | 1789 |
| | | | CoV1 | | | RBC | Human Patient | | FSSYWMSWVRQAPGKGLEWVANIKQ | |
| | | | | | | | | | DGSEKYYVDSVKGRFTISRDNAKNSLYL | |
| | | | | | | | | | QMNSLRAEDTAVYYCARDFNSYQLLWY | |
| | | | | | | | | | YYYGMDVWGQGTTVTVSS | |
| CV16 | Ab | SARS-CoV2 | SARS- | | SARS-CoV2 | S; non- | B-cells; SARS-CoV2 | 711 | EVQLVQSGAEVKKPGESLKISCKGSGYSF | 1790 |
| | | | CoV1 | | | RBD | Human Patient | | TSYWIGWVRQMPGKGLEWMGIIYPGD | |
| | | | | | | | | | SDTRYSPSFQGQVTISADKSISTAYLQWS | |
| | | | | | | | | | SLKASDTAMYYCARQSSFYSSGWYSYG | |
| | | | | | | | | | MDVWGQGTTVTVSS | |
| CV17 | Ab | SARS-CoV2 | SARS- | | SARS-CoV2 | S; non- | B-cells; SARS-CoV2 | 712 | QVQLVQSGAEVKKPGASVKVSCKASGY | 1791 |
| | | | CoV1 | | | RBD | Human Patient | | TFTGYYMHWVRQAPGQGLEWMGWI | |
| | | | | | | | | | NPNSGGTNYAQKFQGRVTMTRDTSIST | |
| | | | | | | | | | AYMELSRLRSDDTAVYYCARVDYGSGSY | |
| | | | | | | | | | GWGWFDPWGQGTLVTVSS | |
| CV18 | Ab | SARS-CoV1, | | | SARS-CoV2 | S; non- | B-cells; SARS-CoV2 | 713 | QVQLVQSGAEVKKPGASVKVSCKVSGY | 1792 |
| | | SARS-CoV2 | | | | RBD | Human Patient | | TLTELSMHWVRQAPGKGLEWMGGFD | |
| | | | | | | | | | PEDGETIYAQKFQGRVTMTEDTSTDTAY | |
| | | | | | | | | | MELSSLRSEDTAVYYCATTSPIVGAITWF | |
| | | | | | | | | | DPWGQGTLVTVSS | |
| CV19 | Ab | SARS-CoV2 | SARS- | | SARS-CoV2 | S; non- | B-cells; SARS-CoV2 | 714 | QVQLVQSGAEVKKPGASVKVSCKASGY | 1793 |
| | | | CoV1 | | | RBD | Human Patient | | TFTGYYMHWVRQAPGQGLEWMGWI | |
| | | | | | | | | | NPNSGGTNYAQKFQGRVTMTRDTSIST | |
| | | | | | | | | | AYMELSRLRSDDTAVYYCAREYYYDSSVY | |
| | | | | | | | | | PYYYYAMDVWGQGTTVTVSS | |
| CV2 | Ab | SARS-CoV2 | SARS- | | SARS-CoV2 | S; non- | B-cells; SARS-CoV2 | 715 | QVQLVESGGGVVQPGRSLRLSCAASGF | 1794 |
| | | | CoV1 | | | RBD | Human Patient | | TFSSYAMHWVRQAPGKGLEWVAVISY | |
| | | | | | | | | | DGSNKYYADSVKGRFTISRDNSKNTLYL | |
| | | | | | | | | | QMNSLRAEDTAVYYCARVRGSYYLFDY | |
| | | | | | | | | | WGQGTLVTVSS | |
| CV21 | Ab | SARS-CoV2 | SARS- | | SARS-CoV2 | S; non- | B-cells; SARS-CoV2 | 716 | EVQLVESGGGLVKPGGSLRLSCAASGFT | 1795 |
| | | | CoV1 | | | RBD | Human Patient | | FSNAWMSWVRQAPGKGLEWVGRIKSK | |
| | | | | | | | | | TDGGTTDYAAPVKGRFTISRDDSKNTLY | |
| | | | | | | | | | LQMNSLKTEDTAVYYCTTDRVYDYIWGS | |
| | | | | | | | | | YRYLDYWGQGTLVTVSS | |
| CV22 | Ab | SARS-CoV1, | | | SARS-CoV2 | S; non- | B-cells; SARS-CoV2 | 717 | EVQLVESGGGLVKPGGSLRLSCAASGFT | 1796 |
| | | SARS-CoV2 | | | | RBD | Human Patient | | FSSYSMNWVRQAPGKGLEWVSSISSSSS | |
| | | | | | | | | | YIYYADSVKGRFTISRDNAKNSLYLQMNS | |
| | | | | | | | | | LRAEDTAVYYCARDRESYDILTGYSMEG | |
| | | | | | | | | | CFDYWGQGTLVTVSS | |
| CV23 | Ab | SARS-CoV2 | SARS- | | SARS-CoV2 | S; non- | B-cells; SARS-CoV2 | 718 | QVQLVQSGAEVKKPGASVKVSCKASGY | 1797 |
| | | | CoV1 | | | RBD | Human Patient | | TFTSYAMHWVRQAPGQRLEWMGWIN | |
| | | | | | | | | | AGNGNTKYSQKFQGRVTITRDTSASTAY | |
| | | | | | | | | | MELSSLRSEDTAVYYCARVWGYCSGGS | |
| | | | | | | | | | CYVDAFDIWGQGTMVTVSS | |
| CV24 | Ab | SARS-CoV2 | SARS- | | SARS-CoV2 | S; non- | B-cells; SARS-CoV2 | 719 | QVQLVQSGAEVKKPGASVKVSCKVSGY | 1798 |
| | | | CoV1 | | | RBD | Human Patient | | TLTELSMHWVRQAPGKGLEWMGGFD | |
| | | | | | | | | | PEDGETIYAQKFQGRVTMTEDTSTDTAY | |
| | | | | | | | | | MELSSLRSEDTAVYYCATAPPYSPPSSWF | |
| | | | | | | | | | DPWGQGTLVTVSS | |
| CV25 | Ab | SARS-CoV2 | SARS- | | SARS-CoV2 | S; non- | B-cells; SARS-CoV2 | 720 | QVQLQESGPGLVKPSQTLSLTCTVSGGSI | 1799 |
| | | | CoV1 | | | RBC | Human Patient | | SSGDYYWSWIRQPPGKGLEWIGYIYYSG | |
| | | | | | | | | | STYYNPSLKSRVTISVDTSKNQFSLKLSSV | |
| | | | | | | | | | TAADTAVYYCARDHHYDFWSGYSSYYYY | |
| | | | | | | | | | GMDVWGQGTTVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CV12 | DVVMTQSPLSLPVTLGQPATISCRSSQ | IGHV3-30 | IGHJ4 | IGKV2-30 | IGKJ1 | 2809 | VRGGVSGPN | 4011 | MQGTHW | Emilie Seydoux et al., |
| | SLVYSDGNTYVNWFQQRPGQSPRRLI | (Human) | (Human) | (Human) | (Human) | | SFDM | | PVT | 2020 |
| | YQVSIRASGVPDRFSGSGSGTDFALKIS | | | | | | | | | (https://www.biorxiv.or |
| | RVEAEDVGVYYCMQGTHWPVTFGQ | | | | | | | | | g/content/10.1101/202 |
| | GTKVEIK | | | | | | | | | 0.05.12.091298v1) |
| CV13 | DIQMTQSPSSLSASVGDRVTITCRASQ | IGHV7-4- | IGHJ4 | IGKV1-39 | IGKJ3 | 2810 | ARASARPGVA | 4012 | QQSYSNPL | Emilie Seydoux et al., |
| | NIDNYLNWYQQKPGKAPKLLIYAASRL | 1 (Human) | (Human) | (Human) | (Human) | | TNLDF | | T | 2020 |
| | HSGVPSRFSGSGSGTDFTLIISSLQPED | | | | | | | | | (https://www.biorxiv.or |
| | LATYYCQQSYSNPLTFGPGTKVDIR | | | | | | | | | g/content/10.1101/202 |
| | | | | | | | | | | 0.05.12.091298v1) |
| CV15 | QSALTQPRSVSGSPGQSVTISCTGTSS | IGHV3-7 | IGHJ6 | IGLV2-11 | IGLJ3 | 2811 | ARDFNSYQLL | 4013 | CSYAGSYT | Emilie Seydoux et al., |
| | DVGGYNYVSWYQQHPGKAPKLMIYD | (Human) | (Human) | (Human) | (Human) | | WYYYYGMDV | | WV | 2020 |
| | VSKRPSGVPDRFSGSKSGNTASLTISGL | | | | | | | | | (https://www.biorxiv.or |
| | QAEDEADYYCCSYAGSYTWVFGGGTK | | | | | | | | | g/content/10.1101/202 |
| | LTVL | | | | | | | | | 0.05.12.091298v1) |
| CV16 | EIVLTQSPGTLSLSPGERATLSCRASQS | IGHV5-51 | IGHJ6 | IGKV3-20 | IGKJ1 | 2812 | ARQSSFYSSG | 4014 | QQYGSSR | Emilie Seydoux et al., |
| | VSSSYLAWYQQKPGQAPRLLIYGASSR | (Human) | (Human) | (Human) | (Human) | | WYSYGMDV | | GT | 2020 |
| | ATGIPDRFSGSGSGTDFTLTISRLEPED | | | | | | | | | (https://www.biorxiv.or |
| | FAVYYCQQYGSSRGTFGQGTKVEIK | | | | | | | | | g/content/10.1101/202 |
| | | | | | | | | | | 0.05.12.091298v1) |
| CV17 | QSALTQPASVSGSPGQSITISCTGTSSD | IGHV1-2 | IGHJ5 | IGLV2-23 | IGLJ1 | 2813 | ARVDYGSGSY | 4015 | CSYAGSST | Emilie Seydoux et al., |
| | VGSYNLVSWYQQHPGKAPKLMIYEGS | (Human) | (Human) | (Human) | (Human) | | GWGWFDP | | YV | 2020 |
| | KRPSGVSNRFSGSKSGNTASLTISGLQ | | | | | | | | | (https://www.biorxiv.or |
| | AEDEADYYCCSYAGSSTYVFGTGTKVT | | | | | | | | | g/content/10.1101/202 |
| | VL | | | | | | | | | 0.05.12.091298v1) |
| CV18 | QSVLTQPPSVSAAPGQKVTISCSGSSS | IGHV1-24 | IGHJ5 | IGLV1-51 | IGLJ3 | 2814 | ATTSPIVGA | 4016 | GTWDSSLS | Emilie Seydoux et al., |
| | NIGNNYVSWYQQLPGTAPKLLIYDNN | (Human) | (Human) | (Human) | (Human) | | ITWFDP | | AGPV | 2020 |
| | KRPSGIPDRFSGSKSGTSATLGITGLQT | | | | | | | | | (https://www.biorxiv.or |
| | GDEADYYCGTWDSSLSAGPVFGGGT | | | | | | | | | g/content/10.1101/202 |
| | KLTVL | | | | | | | | | 0.05.12.091298v1) |
| CV19 | EIVLTQSPGTLSLSPGERATLSCRASQS | IGHV1-2 | IGHJ6 | IGKV3-20 | IGKJ2 | 2815 | AREYYYDSSV | 4017 | QQYGSSPP | Emilie Seydoux et al., |
| | VSSSYLAWYQQKPGQAPRLLIYGASSR | (Human) | (Human) | (Human) | (Human) | | YPYYYYAMDV | | KYT | 2020 |
| | ATGIPDRFSGSGSGTDFTLTISRLEPED | | | | | | | | | (https://www.biorxiv.or |
| | FAVYYCQQYGSSPPKYTFGQGTKLEIK | | | | | | | | | g/content/10.1101/202 |
| | | | | | | | | | | 0.05.12.091298v1) |
| CV2 | EIVMTQSPATLSVSPGERATLSCRASQ | IGHV3-30 | IGHJ4 | IGKV3-15 | IGKJ4 | 2816 | ARVRGSYYLF | 4018 | QQYNNWP | Emilie Seydoux et al., |
| | SVSSNLAWYQQKPGQAPRLLIYGAST | (Human) | (Human) | (Human) | (Human) | | DY | | PSLT | 2020 |
| | RATGIPARFSGSGSGTEFTLTISSLQSE | | | | | | | | | (https://www.biorxiv.or |
| | DFAVYYCQQYNNWPPSLTFGGGTKV | | | | | | | | | g/content/10.1101/202 |
| | EIK | | | | | | | | | 0.05.12.091298v1) |
| CV21 | EIVLTQSPATLSLSPGERATLSCRASQS | IGHV3-15 | IGHJ4 | IGKV3-11 | IGKJ4 | 2817 | TTDRVYDYIW | 4019 | QQRSNWP | Emilie Seydoux et al., |
| | VSSYLAWYQQKPGQAPRLLIYDASNR | (Human) | (Human) | (Human) | (Human) | | GSYRYLDY | | LT | 2020 |
| | ATGIPARFSGSGSGTDFTLTISSLEPEDF | | | | | | | | | (https://www.biorxiv.or |
| | AVYYCQQRSNWPLTFGGGTKVEIK | | | | | | | | | g/content/10.1101/202 |
| | | | | | | | | | | 0.05.12.091298v1) |
| CV22 | QLVLTQSPSASASLGASVKLTCTLSSGH | IGHV3-21 | IGHJ4 | IGLV4-69 | IGLJ3 | 2818 | ARDRESYDIL | 4020 | QTWGTGI | Emilie Seydoux et al., |
| | SSYAIAWHQQQPEKGPRYLMKLNSD | (Human) | (Human) | (Human) | (Human) | | TGYSMEGCFD | | RV | 2020 |
| | GSHSKGDGIPDRFSGSSSGAERYLTISS | | | | | | Y | | | (https://www.biorxiv.or |
| | LQSEDEADYYCQTWGTGIRVFGGGTK | | | | | | | | | g/content/10.1101/202 |
| | LTVL | | | | | | | | | 0.05.12.091298v1) |
| CV23 | SYELTQPPSVSVSPGQTARITCSGDALP | IGHV1-3 | IGHJ3 | IGLV3-25 | IGLJ3 | 2819 | ARVWGYCSG | 4021 | QSADSSGT | Emilie Seydoux et al., |
| | KQYAYWYQQKPGQAPVLVIYKDSERP | (Human) | (Human) | (Human) | (Human) | | GSCYVDAFDI | | YVV | 2020 |
| | SGIPERFSGSSSGTTVTLTISGVQAEDE | | | | | | | | | (https://www.biorxiv.or |
| | ADYYCQSADSSGTYVVFGGGTKLTVL | | | | | | | | | g/content/10.1101/202 |
| | | | | | | | | | | 0.05.12.091298v1) |
| CV24 | QSVLTQPPSVSAAPGQKVTISCSGSSS | IGHV1-24 | IGHJ5 | IGLV1-51 | IGLJ1 | 2820 | ATAPPYSPPS | 4022 | GTWDSSL | Emilie Seydoux et al., |
| | NIGNNYVSWYQQLPGTAPKLLIYDNN | (Human) | (Human) | (Human) | (Human) | | SWFDP | | SASYV | 2020 |
| | KRPSGIPDRFSGSKSGTSATLGITGLQT | | | | | | | | | (https://www.biorxiv.or |
| | GDEADYYCGTWDSSLSASYVFGTGTK | | | | | | | | | g/content/10.1101/202 |
| | VTVL | | | | | | | | | 0.05.12.091298v1) |
| CV25 | EIVMTQSPATLSVSPGERATLSCRASQ | IGHV4-30- | IGHJ6 | IGKV3-15 | IGKJ2 | 2821 | ARDHHYDFW | 4023 | QQYNNWP | g/content/10.1101/202 |
| | SVSSNLAWYQQKPGQAPRLLIYGAST | 4 | (Human) | (Human) | (Human) | | SGYSSYYYYG | | YT | Emilie Seydoux et al., |
| | RATGIPARFSGSGSGTEFTLTISSLQSE | (Human) | | | | | MDV | | | 2020 |
| | DFAVYYCQQYNNWPYTFGQGTKLEIK | | | | | | | | | (https://www.biorxiv.or |
| | | | | | | | | | | 0.05.12.091298v1) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CV26 | Ab | SARS-CoV1, | | | SARS-CoV2 | S; non- | B-cells; SARS-CoV2 | 721 | QVQLVESGGGVVQPGRSLRLSCAASGF | 1800 |
| | | SARS-CoV2 | | | | RBD | Human Patient | | TFSSYAMHWVRQAPGKGLEWVAVISY | |
| | | | | | | | | | DGSNKYYADSVKGRFTISRDNSKNTLYL | |
| | | | | | | | | | QMNSLRAEDTAVYYCARDEAYYDILTGY | |
| | | | | | | | | | INAPKNYYYYGMDVWGQGTTVTVSS | |
| CV27 | Ab | SARS-CoV2 | SARS- | | SARS-CoV2 | S; non- | B-cells; SARS-CoV2 | 722 | QVQLVESGGGVVQPGRSLRLSCAASGF | 1801 |
| | | | CoV1 | | | RBD | Human Patient | | TFSSYAMHWVRQAPGKGLEWVALISYD | |
| | | | | | | | | | GSNKYYADSVKGRFTISRDNSKNTLYLQ | |
| | | | | | | | | | MNSLRAEDTAVYYCARSFGGSYYYGMD | |
| | | | | | | | | | VWGQGTTVTVSS | |
| CV3 | Ab | SARS-CoV1, | | | SARS-CoV2 | S; non- | B-cells; SARS-CoV2 | 723 | QVQLVQSGSELKKPGASVKLSCQASGYS | 1802 |
| | | SARS-CoV2 | | | | RBC | Human Patient | | FTNHAMNWVRQAPGQGLEWMGWIN | |
| | | | | | | | | | TNTGNPTYAQGFTGRFVFSLDTSVSTTYL | |
| | | | | | | | | | HISSLKAEDTAVYYCARASARPGVATNL | |
| | | | | | | | | | DFWGQGTLVVVSS | |
| CV30 | Ab | SARS-CoV2 | SARS- | SARS-CoV2 | | S; RBD | B-cells; SARS-CoV2 | 724 | EVQLVESGGGLIQPGGSLRLSCAASGVI | 1803 |
| | | | CoV1 | | | | Human Patient | | VSSNYMSWVRQAPGKGLEWVSVIYSG | |
| | | | | | | | | | GSTYYADSVKGRFTISRDNSKNTLYLQM | |
| | | | | | | | | | NSLRAEDTAVYYCARDLDVSGGMDVW | |
| | | | | | | | | | GQGTTVTVSS | |
| CV31 | Ab | SARS-CoV1, | | | SARS-CoV2 | S; non- | B-cells; SARS-CoV2 | 725 | QVQLVQSGAEVKKPGASVKVSCKVSGY | 1804 |
| | | SARS-CoV2 | | | | RBD | Human Patient | | TLTELSMHWVRQAPGKGLEWMGGFD | |
| | | | | | | | | | PEDGETIYAQKFQGRVTMTEDTSTDTAY | |
| | | | | | | | | | MELSSLRSEDTAVYYCATAPPYSPPSSWF | |
| | | | | | | | | | DPWGQGTLVTVSS | |
| CV32 | Ab | SARS-CoV2 | SARS- | | SARS-CoV2 | S; non- | B-cells; SARS-CoV2 | 726 | QVQLVQSGAEVKKPGASVKVSCKASGY | 1805 |
| | | | CoV1 | | | RBD | Human Patient | | TFTGYYMHWVRQAPGQGLEWMGWI | |
| | | | | | | | | | NPNSDVTNYAQKFQGRVTMTRDTSIST | |
| | | | | | | | | | AYMELSRLRSDDTAVYYCAREARDYYGS | |
| | | | | | | | | | GSLDYWGQGTLVTVSS | |
| CV33 | Ab | SARS-CoV2 | SARS- | | SARS-CoV2 | S; non- | B-cells; SARS-CoV2 | 727 | QVQLVQSGAEVKKPGASVKVSCKASGY | 1806 |
| | | | CoV1 | | | RBD | Human Patient | | TFTSYDISWVRQAPGQGLEWMGWISA | |
| | | | | | | | | | YNGNTNYAQKLQGRVTMTTDTSTSTAY | |
| | | | | | | | | | MELRSLRSDDTAVYYCARDSVAGIYYYY | |
| | | | | | | | | | GMDVWGQGTTVTVSS | |
| CV34 | Ab | SARS-CoV2 | SARS- | | SARS-CoV2 | S; non- | B-cells; SARS-CoV2 | 728 | QVQLVESGGGVVQPGRSLRLSCAASGF | 1807 |
| | | | CoV1 | | | RBD | Human Patient | | TFSSYAMHWVRQAPGKGLEWVAVISY | |
| | | | | | | | | | DGSNKYYADSVKGRFTISRDNSKNTLYL | |
| | | | | | | | | | QMNSLRAEDTAVYYCARSYGGSYYYGM | |
| | | | | | | | | | DVWGQGTTVTVSS | |
| CV35 | Ab | SARS-CoV2 | SARS- | | SARS-CoV2 | S; non- | B-cells; SARS-CoV2 | 729 | QVQLQESGPGLVKPSETLSLTCTVSGYSI | 1808 |
| | | | CoV1 | | | RBD | Human Patient | | SSGYYWGWIRQPPGKGLEWIGSIYHSG | |
| | | | | | | | | | STYYNPSLKSRVTISVDTSKNQFSLKLSSV | |
| | | | | | | | | | TAADTAVYYCARTPLSLRLRYNWYFDLW | |
| | | | | | | | | | GRGTLVTVSS | |
| CV36 | Ab | SARS-CoV2 | SARS- | | SARS-CoV2 | S; non- | B-cells; SARS-CoV2 | 730 | QVQLVQSGAEVKKPGASVKVSCKASGY | 1809 |
| | | | CoV1 | | | RBD | Human Patient | | TFTGYYMHWVRQAPGQGLEWMGWI | |
| | | | | | | | | | NPNSGGTNYVQKFQGRVTMTRDTSIST | |
| | | | | | | | | | AYMELNRLRSDDTAVYFCARDLTTTAGT | |
| | | | | | | | | | DYYYGMDVWGQGTTVTVSS | |
| CV37 | Ab | SARS-CoV1, | | | SARS-CoV2 | S; non- | B-cells; SARS-CoV2 | 731 | QVQLVQSGAEVKKPGASVKVSCKASGY | 1810 |
| | | SARS-CoV2 | | | | RBD | Human Patient | | TFTSYGISWVRQAPGQGLEWMGWISA | |
| | | | | | | | | | YNGNTNYAQKLQGRVTMTTDTSTSTAY | |
| | | | | | | | | | MELRSLRSDDTAVYYCARARVAYDYIW | |
| | | | | | | | | | GSYRYKAFDYWGQGTLVTVSS | |
| CV38 | Ab | SARS-CoV1, | | | SARS-CoV2 | S; non- | B-cells; SARS-CoV2 | 732 | QVQLVESGGGVVQPGRSLRLSCAASGF | 1811 |
| | | SARS-CoV2 | | | | RBD | Human Patient | | TFSSYAMHWVRQAPGKGLEWVAVISY | |
| | | | | | | | | | DGSNKYYADSVKGRFTISRDNSKNTLYL | |
| | | | | | | | | | QMNSLRAEDTAVYYCARAQTAHYSSSF | |
| | | | | | | | | | DYWGQGTLVTVSS | |
| CV38-113 | Ab | SARS-CoV2 | SARS- | SARS-CoV2 | | S; RBD | B-cells; SARS-CoV2 | | ND | |
| | | | CoV1 | | | | Human Patient | | | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CV26 | DIQMTQSPSSLSASVGDRVTITCRASQ | IGHV3-30- | (Human) | IGKV1-17 | IGKJ3 | 2822 | ARDEAYYDILT | 4024 | LQHNSYPF | Emilie Seydoux et al., |
| | GIRNDLGWYQQKPGKAPKRLIYAASSL | 3 | IGHJ6 | (Human) | (Human) | | GYINAPKNYY | | T | 2020 |
| | QSGVPSRFSGSGSGTEFTLTISSLQPED | (Human) | | | | | YYGMDV | | | (https://www.biorxiv.or |
| | FATYYCLQHNSYPFTFGPGTKVDIK | | | | | | | | | g/content/10.1101/202 |
| | | | | | | | | | | 0.05.12.091298v1) |
| CV27 | QSALTQPASVSGSPGQSITISCTGTSSD | IGHV3-30 | IGHJ6 | IGLV2-14 | IGLJ1 | 2823 | ARSFGGSYYY | 4025 | SSYTSSSTP | Emilie Seydoux et al., |
| | VGGYNYVSWYQQHPGKAPKLMIYDV | (Human) | (Human) | (Human) | (Human) | | GMDV | | YV | 2020 |
| | SNRPSGVSNRFSGSKSGNTASLTISGL | | | | | | | | | (https://www.biorxiv.or |
| | QAEDEADYYCSSYTSSSTPYVFGTGTK | | | | | | | | | g/content/10.1101/202 |
| | VTVL | | | | | | | | | 0.05.12.091298v1) |
| CV3 | DIQMTQSPSSLSASVGDRVTITCRASQ | IGHV7-4- | IGHJ4 | IGKV1-39 | IGKJ3 | 2824 | ARASARPGVA | 4026 | QQSYSNPL | Emilie Seydoux et al., |
| | NIDNYLNWYQQKPGKAPKLLIYAASRL | 1 (Human) | (Human) | (Human) | (Human) | | TNLDF | | T | 2020 |
| | HSGVPSRFSGSGSGTDFTLIISSLQPED | | | | | | | | | (https://www.biorxiv.or |
| | LATYYCQQSYSNPLTFGPGTKVDIR | | | | | | | | | g/content/10.1101/202 |
| | | | | | | | | | | 0.05.12.091298v1) |
| CV30 | EIVLTQSPGTLSLSPGERATLSCRASQS | IGHV3-53 | IGHJ6 | IGKV3-20 | IGKJ2 | 2825 | ARDLDVSGG | 4027 | QQYGSSP | Emilie Seydoux et al., |
| | VSSSYLAWYQQKPGQAPRLLIYGASSR | (Human) | (Human) | (Human) | (Human) | | MDV | | QT | 2020 |
| | ATGIPDRFSGSGSGTDFTLTISRLEPED | | | | | | | | | (https://www.biorxiv.or |
| | FAVYYCQQYGSSPQTFGQGTKLEIK | | | | | | | | | g/content/10.1101/202 |
| | | | | | | | | | | 0.05.12.091298v1); Nich |
| | | | | | | | | | | olas Hurlburt et al., |
| | | | | | | | | | | 2020 |
| | | | | | | | | | | (https://www.biorxiv.or |
| | | | | | | | | | | g/content/10.1101/202 |
| | | | | | | | | | | 0.06.12.148692v1) |
| CV31 | QSVLTQPPSVSAAPGQKVTISCSGSSS | IGHV1-24 | IGHJ5 | IGLV1-51 | IGLJ1 | 2826 | ATAPPYSPPSS | 4028 | GTWDSSLS | Emilie Seydoux et al., |
| | NIGNNYVSWYQQLPGTAPKLLIYDNN | (Human) | (Human) | (Human) | (Human) | | WFDP | | ASYV | 2020 |
| | KRPSGIPDRFSGSKSGTSATLGITGLQT | | | | | | | | | (https://www.biorxiv.or |
| | GDEADYYCGTWDSSLSASYVFGTGTK | | | | | | | | | g/content/10.1101/202 |
| | VTVL | | | | | | | | | 0.05.12.091298v1) |
| CV32 | QSVLTQPPSVSAAPGQKVTISCSGSSS | IGHV1-2 | IGHJ4 | IGLV1-51 | IGLJ3 | 2827 | AREARDYYGS | 4029 | GTWDSSLS | Emilie Seydoux et al., |
| | NIGNNYVSWYQQLPGTAPKLLIYDNN | (Human) | (Human) | (Human) | (Human) | | GSLDY | | AVV | 2020 |
| | KRPSGIPDRFSGSKSGTSATLGITGLQT | | | | | | | | | (https://www.biorxiv.or |
| | GDEADYYCGTWDSSLSAVVFGGGTKL | | | | | | | | | g/content/10.1101/202 |
| | TVL | | | | | | | | | 0.05.12.091298v1) |
| CV33 | QSVLTQPPSVSGAPGQRVTISCTGSSS | IGHV1-18 | IGHJ6 | IGLV1-40 | IGLJ3 | 2828 | ARDSVAGIYY | 4030 | QSYDSSLS | Emilie Seydoux et al., |
| | NIGAGYDVHWYQQLPGTAPKLLIYGN | (Human) | (Human) | (Human) | (Human) | | YYGMDV | | GPVV | 2020 |
| | SNRPSGVPDRFSGSKSGTSASLAITGL | | | | | | | | | (https://www.biorxiv.or |
| | QAEDEADYYCQSYDSSLSGPVVFGGG | | | | | | | | | g/content/10.1101/202 |
| | TKLTVL | | | | | | | | | 0.05.12.091298v1) |
| CV34 | SYELTQPHSVSVATAQMARITCGGNN | IGHV3-30- | IGHJ6 | IGLV3-12 | IGLJ3 | 2829 | ARSYGGSYYY | 4031 | QVWDSSS | Emilie Seydoux et al., |
| | IGSKAVHWYQQKPGQDPVLVIYSDSN | 3 | (Human) | (Human) | (Human) | | GMDV | | DHVV | 2020 |
| | RPSGIPERFSGSNPGNTATLTISRIEAG | (Human) | | | | | | | | (https://www.biorxiv.or |
| | DEADYYCQVWDSSSDHVVFGGGTKL | | | | | | | | | g/content/10.1101/202 |
| | TVL | | | | | | | | | 0.05.12.091298v1) |
| CV35 | QSVLTQPPSASGTPGQRVTISCSGSSS | IGHV4-38 | IGHJ2 | IGLV1-44 | IGLJ3 | 2830 | ARTPLSLRLRY | 4032 | AAWDDSL | Emilie Seydoux et al., |
| | NIGSNTVNWYQQLPGTAPKLLIYSNN | (Human) | (Human) | (Human) | (Human) | | NWYFDL | | NGPV | 2020 |
| | QRPSGVPDRFSGSKSGTSASLAISGLQ | | | | | | | | | (https://www.biorxiv.or |
| | SEDEADYYCAAWDDSLNGPVFGGGT | | | | | | | | | g/content/10.1101/202 |
| | KLTVL | | | | | | | | | 0.05.12.091298v1) |
| CV36 | SYELTQPPSVSVSPGQTARITCSGDALP | IGHV1-2 | IGHJ6 | IGLV3-25 | IGLJ1 | 2831 | ARDLTTTAGT | 4033 | QSADSSGT | Emilie Seydoux et al., |
| | KQYAYWYQQKPGQAPVLVIYKDTERP | (Human) | (Human) | (Human) | (Human) | | DYYYGMDV | | YMI | 2020 |
| | SGIPERFSGSSSGTTVTLTISGVQAEDE | | | | | | | | | (https://www.biorxiv.or |
| | ADYYCQSADSSGTYMIFGTGTKVTVL | | | | | | | | | g/content/10.1101/202 |
| | | | | | | | | | | 0.05.12.091298v1) |
| CV37 | DIQMTQSPSSLSASVGDRVTITCQASQ | IGHV1-18 | IGHJ4 | IGKV1-33 | IGKJ3 | 2832 | ARARVAYDYI | 4034 | QQYDNLP | Emilie Seydoux et al., |
| | DISNYLNWYQQKPGKAPKLLIYDASNL | (Human) | (Human) | (Human) | (Human) | | WGSYRYKAF | | R | 2020 |
| | ETGVPSRFSGSGSGTDFTFTISSLQPED | | | | | | DY | | | (https://www.biorxiv.or |
| | IATYYCQQYDNLPRFGPGTKVDIKR | | | | | | | | | g/content/10.1101/202 |
| | | | | | | | | | | 0.05.12.091298v1) |
| CV38 | EIVLTQSPATLSLSPGERATLSCRASQS | IGHV3-30 | IGHJ4 | IGKV3-11 | IGKJ5 | 2833 | ARAQTAHYSS | 4035 | QQRSNWP | Emilie Seydoux et al., |
| | VSSYLAWYQQKPGQAPRLLIYDASNR | (Human) | (Human) | (Human) | (Human) | | SFDY | | PIT | 2020 |
| | ATGIPARFSGSGSGTDFTLTISSLEPEDF | | | | | | | | | (https://www.biorxiv.or |
| | AVYYCQQRSNWPPITFGQGTRLEIK | | | | | | | | | g/content/10.1101/202 |
| | | | | | | | | | | 0.05.12.091298v1) |
| CV38-113 | ND | IGHV3-53 | IGHJ4 | ND | ND | 2834 | ARGGRLADA | 4036 | QQYDNLPS | Jakob Kreye et al., 2020 |
| | | (Human) | (Human) | | | | AGDY | | WT | (https://www.biorxiv.or |
| | | | | | | | | | | g/content/10.1101/202 |
| | | | | | | | | | | 0.08.15.252320v1.full.p |
| | | | | | | | | | | df) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CV38-139 | Ab | SARS-CoV2 | SARS- | SARS-CoV2 | | S; RBD | B-cells; SARS-CoV2 | | ND | |
| | | | CoV1 | | | | Human Patient | | | |
| CV38-142 | Ab | SARS-CoV1, | | SARS-CoV2 | | S; RBD | B-cells; SARS-CoV2 | | ND | |
| | | SARS-CoV2 | | | | | Human Patient | | | |
| CV38-183 | Ab | SARS-CoV2 | SARS- | SARS-CoV2 | | S; RBD | B-cells; SARS-CoV2 | | ND | |
| | | | CoV1 | | | | Human Patient | | | |
| CV38-221 | Ab | SARS-CoV2 | SARS- | SARS-CoV2 | | S; RBD | B-cells; SARS-CoV2 | | ND | |
| | | | CoV1 | | | | Human Patient | | | |
| CV39 | Ab | SARS-CoV2 | SARS- | | SARS-CoV2 | S; non- | B-cells; SARS-CoV2 | 733 | QVQLVESGGGVVQPGRSMRLSCAASG | 1812 |
| | | | CoV1 | | | RBD | Human Patient | | FNLSSYAMHWVRQASGKGLEWVSLISY | |
| | | | | | | | | | DGSIKYYADSVKGRFTVSGDNSKNTLFL | |
| | | | | | | | | | QMSSLRADDSALYYCVRGGVSGPNAFD | |
| | | | | | | | | | IWGQGTTVTVSS | |
| CV4 | Ab | SARS-CoV1, | | | SARS-CoV2 | S; non- | B-cells; SARS-CoV2 | 734 | QVQLVESGGGVVQPGRSLRLSCAASGF | 1813 |
| | | SARS-CoV2 | | | | RBD | Human Patient | | TFSSYAMHWVRQAPGKGLEWVAVISY | |
| | | | | | | | | | DGSNKYYADSVKGRFTISRDNSKNTLYL | |
| | | | | | | | | | QMNSLRAEDTAVYYCARSISGSYLGAFD | |
| | | | | | | | | | YWGQGTLVTVSS | |
| CV40 | Ab | SARS-CoV1, | | | SARS-CoV2 | S; non- | B-cells; SARS-CoV2 | 735 | QVQLVQSGAEVKKPGASVKVSCKASGY | 1814 |
| | | SARS-CoV2 | | | | RBD | Human Patient | | TFSSYGISWVRQDPGQGLEWMGWISA | |
| | | | | | | | | | YNGNTNYAQKLQGRVTMTTDTSTSTAY | |
| | | | | | | | | | MELRSLRSDDTAVYYCARVGLWWLGH | |
| | | | | | | | | | PDAFDIWGQGTMVTVSS | |
| CV41 | Ab | SARS-CoV2 | SARS- | | SARS-CoV2 | S; non- | B-cells; SARS-CoV2 | 736 | QVQLVESGGGVVQPGRSLRLSCAASGF | 1815 |
| | | | CoV1 | | | RBD | Human Patient | | TFSSYAMHWVRQAPGKGLEWVAVISY | |
| | | | | | | | | | DGSNKYYADSVKGRFTISRDNSKNTLYL | |
| | | | | | | | | | QMNSLRAEDTAVYYCARTKGGSYFAPF | |
| | | | | | | | | | DYWGQGTLVTVSS | |
| CV42 | Ab | SARS-CoV2 | SARS- | | SARS-CoV2 | S; non- | B-cells; SARS-CoV2 | 737 | QVQLVQSGAEVKKPGASVKVSCKASGY | 1816 |
| | | | CoV1 | | | RBD | Human Patient | | TFTSYGISWVRQAPGQGLEWMGWISA | |
| | | | | | | | | | YNGNTNYAQKVQGRVTMTTDTSTSTAY | |
| | | | | | | | | | MELRSLRSDDTAVYYCARDRGYAATFG | |
| | | | | | | | | | VFDYWGQGTLVTVSS | |
| CV43 | Ab | SARS-CoV2 | SARS- | | SARS-CoV2 | S; RBD | B-cells; SARS-CoV2 | 738 | QVQLVESGGGVVQPGRSLRLSCAASGF | 1817 |
| | | | CoV1 | | | | Human Patient | | TFSSYAMHWVRQAPGKGLEWVAVISY | |
| | | | | | | | | | DGSNKYYADSVKGRFTISRDNSKNTLYL | |
| | | | | | | | | | QMNSLRAEDTAVYYCARVTVVHFDYW | |
| | | | | | | | | | GQGTLVTVSS | |
| CV44 | Ab | SARS-CoV2 | SARS- | | SARS-CoV2 | S; non- | B-cells; SARS-CoV2 | 739 | QVQLVQSGAEVKKPGASVKVSCKASGY | 1818 |
| | | | CoV1 | | | RBD | Human Patient | | TFTSYYMHWVRQAPGQGLEWMGIINP | |
| | | | | | | | | | SGGSTSYAQKFQGRVTMTRDTSTSTVY | |
| | | | | | | | | | MELSSLRSEDTAVYYCARDLTSTSSSPYSY | |
| | | | | | | | | | YYGMDVWGQGTTVTVSS | |
| CV45 | Ab | SARS-CoV1, | | | SARS-CoV2 | S; non- | B-cells; SARS-CoV2 | 740 | QVQLVQSGAEVKKPGASVKVSCKASGY | 1819 |
| | | SARS-CoV2 | | | | RBD | Human Patient | | TFTSYGISWVRQAPGQGLEWMGWISA | |
| | | | | | | | | | YNGNTNYAQKLQGRVTMTTDTSTSTAY | |
| | | | | | | | | | MELRSLRSDDTAVYYCARVTVEAIFGVVI | |
| | | | | | | | | | LPLKNWFDPWGQGTLVTVSS | |
| CV46 | Ab | SARS-CoV2 | SARS- | | SARS-CoV2 | S; non- | B-cells; SARS-CoV2 | 741 | QVQLVESGGGVVQPGRSLRLSCAASGF | 1820 |
| | | | CoV1 | | | RBD | Human Patient | | NLSNYAMHWVRQASGKGLEWVSLISY | |
| | | | | | | | | | DGSIKYYTDSVKGRFTVSGDNSKNTLFLQ | |
| | | | | | | | | | MNSLRPDDSALYYCVRGGVSGPNSFD | |
| | | | | | | | | | MWGQGTTVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CV38-139 | ND | IGHV3-53 | IGHJ4 | ND | ND | 2835 | ARGHYDLFDY | 4037 | QQLNSYPP | Jakob Kreye et al., 2020 |
| | | (Human) | (Human) | | | | | | GT | (https://www.biorxiv.or |
| | | | | | | | | | | g/content/10.1101/202 |
| | | | | | | | | | | 0.08.15.252320v1.full.p |
| | | | | | | | | | | df) |
| CV38-142 | ND | IGHV5-51 | IGHJ4 | ND | ND | 2836 | ARIRGVYSSG | 4038 | QQSYSTPR | Jakob Kreye et al., 2020 |
| | | (Human) | (Human) | | | | WIGGDY | | QWT | (https://www.biorxiv.or |
| | | | | | | | | | | g/content/10.1101/202 |
| | | | | | | | | | | 0.08.15.252320v1.full.p |
| | | | | | | | | | | df) |
| CV38-183 | ND | IGHV3-53 | IGHJ6 | ND | ND | 2837 | ARGDGWDN | 4039 | QSYDSSLS | Jakob Kreye et al., 2020 |
| | | (Human) | (Human) | | | | YYYGMDV | | GSV | (https://www.biorxiv.or |
| | | | | | | | | | | g/content/10.1101/202 |
| | | | | | | | | | | 0.08.15.252320v1.full.p |
| | | | | | | | | | | df) |
| CV38-221 | ND | IGHV3-66 | IGHJ4 | ND | ND | 2838 | ARGFGDYYFD | 4040 | QQLYT | Jakob Kreye et al., 2020 |
| | | (Human) | (Human) | | | | Y | | | (https://www.biorxiv.or |
| | | | | | | | | | | g/content/10.1101/202 |
| | | | | | | | | | | 0.08.15.252320v1.full.p |
| | | | | | | | | | | df) |
| CV39 | DVVMTQSPLSLPVTLGQPASISCRSSQ | IGHV3-30 | IGHJ3 | IGKV2-30 | IGKJ1 | 2839 | VRGGVSGPN | 4041 | MQGTHW | Emilie Seydoux et al., |
| | SLVYSDGNTYVNWFQQRPGQSPRRLI | (Human) | (Human) | (Human) | (Human) | | AFDI | | PVT | 2020 |
| | YKVSNRDSGVPDRFSGSGSGTDFALKI | | | | | | | | | (https://www.biorxiv.or |
| | SRVEAEDVGVYYCMQGTHWPVTFGQ | | | | | | | | | g/content/10.1101/202 |
| | GTKVEIK | | | | | | | | | 0.05.12.091298v1) |
| CV4 | DIQMTQSPSTLSASVGDRVTITCRASQ | IGHV3-30 | IGHJ4 | IGKV1-5 | IGKJ2 | 2840 | ARSISGSYLGA | 4042 | QQYNSYT | Emilie Seydoux et al., |
| | SISSWLAWYQQKPGKAPKLLIYKASSL | (Human) | (Human) | (Human) | (Human) | | FDY | | | 2020 |
| | ESGVPSRFSGSGSGTEFTLTISSLQPDD | | | | | | | | | (https://www.biorxiv.or |
| | FATYYCQQYNSYTFGQGTKLEIK | | | | | | | | | g/content/10.1101/202 |
| | | | | | | | | | | 0.05.12.091298v1) |
| CV40 | DIQMTQSPSSLSASVGDRVTITCRASQ | IGHV1-18 | IGHJ3 | IGKV1-17 | IGKJ4 | 2841 | ARVGLWWL | 4043 | LQHNSYPL | Emilie Seydoux et al., |
| | GIRNDLGWYQQKPGKAPKRLIYAASSL | (Human) | (Human) | (Human) | (Human) | | GHPDAFDI | | T | 2020 |
| | QSGVPSRFSGSGSGTEFTLTISSLQPED | | | | | | | | | (https://www.biorxiv.or |
| | FATYYCLQHNSYPLTFGGGTKVEIK | | | | | | | | | g/content/10.1101/202 |
| | | | | | | | | | | 0.05.12.091298v1) |
| CV41 | EIVMTQSPATLSVSPGERATLSCRASQ | IGHV3-30 | IGHJ4 | IGKV3-15 | IGKJ4 | 2842 | ARTKGGSYFA | 4044 | QQYNNWP | Emilie Seydoux et al., |
| | SVSSNLAWYQQKPGQAPRLLIYGAST | (Human) | (Human) | (Human) | (Human) | | PFDY | | LT | 2020 |
| | RATGIPARFSGSGSGTEFTLTISSLQSE | | | | | | | | | (https://www.biorxiv.or |
| | DFAVYYCQQYNNWPLTFGGGTKVEIK | | | | | | | | | g/content/10.1101/202 |
| | | | | | | | | | | 0.05.12.091298v1) |
| CV42 | DIQMTQSPSSLSASVGDRVTITCRASQ | IGHV1-18 | IGHJ4 | IGKV1-39 | IGKJ3 | 2843 | ARDRGYAATF | 4045 | QQTYITAF | Emilie Seydoux et al., |
| | SISSYLNWYQQKPGKAPKFLIYAASSLQ | (Human) | (Human) | (Human) | (Human) | | GVFDY | | T | 2020 |
| | SGVPSRFSGSGSGTDFTLTISSLQPEDF | | | | | | | | | (https://www.biorxiv.or |
| | ATYYCQQTYITAFTFGPGTKVDIK | | | | | | | | | g/content/10.1101/202 |
| | | | | | | | | | | 0.05.12.091298v1) |
| CV43 | NFMLTQPHSVSESPGKTVTISCTGSSG | IGHV3-30 | IGHJ4 | IGLV6-57 | IGLJ3 | 2844 | ARVTVVHFDY | 4046 | QSYDSSN | Emilie Seydoux et al., |
| | SIASNYVQWYQQRPGSAPTTVIYEDN | (Human) | (Human) | (Human) | (Human) | | | | WV | 2020 |
| | QRPSGVPDRFSGSIDSSSNSASLTISGL | | | | | | | | | (https://www.biorxiv.or |
| | KTEDEADYYCQSYDSSNWVFGGGTKL | | | | | | | | | g/content/10.1101/202 |
| | TVL | | | | | | | | | 0.05.12.091298v1) |
| CV44 | SYELTQPPSVSVSPGQTARITCSGDALP | IGHV1-46 | IGHJ6 | IGLV3-25 | IGLJ3 | 2845 | ARDLTSTSSSP | 4047 | QSADSSGT | Emilie Seydoux et al., |
| | KQYAYWYQQKPGQAPVVVIYKDSERP | (Human) | (Human) | (Human) | (Human) | | YSYYYGMDV | | YVV | 2020 |
| | SGIPERFSGSSSGTTVTLTISGVQAEDE | | | | | | | | | (https://www.biorxiv.or |
| | ADYYCQSADSSGTYVVFGGGTKLTVL | | | | | | | | | g/content/10.1101/202 |
| | | | | | | | | | | 0.05.12.091298v1) |
| CV45 | QSVLTQPPSVSGAPGQRVTISCTGSSS | IGHV1-18 | IGHJ5 | IGLV1-40 | IGLJ1 | 2846 | ARVTVEAIFG | 4048 | QSYDSSLT | Emilie Seydoux et al., |
| | NIGAGYDVHWYQQLPGTAPKLLIYGN | (Human) | (Human) | (Human) | (Human) | | VVILPLKNWF | | LYV | 2020 |
| | SNRPSGVPDRFSGSKSGTSASLAITGL | | | | | | DP | | | (https://www.biorxiv.or |
| | QAEDEADYYCQSYDSSLTLYVFGTGTK | | | | | | | | | g/content/10.1101/202 |
| | VTVL | | | | | | | | | 0.05.12.091298v1) |
| CV46 | DVVMTQSPLSLPVTLGQPASISCRSSQ | IGHV3-30 | IGHJ4 | IGKV2-30 | IGKJ1 | 2847 | VRGGVSGPN | 4049 | MQGTHW | Emilie Seydoux et al., |
| | SLVYSDGNTYVNWFQQRPGQSPRRLI | (Human) | (Human) | (Human) | (Human) | | SFDM | | PVT | 2020 |
| | YQVSIRASGVPDRFSGSGSGTDFALKIS | | | | | | | | | (https://www.biorxiv.or |
| | RVEAEDVGVYYCMQGTHWPVTFGQ | | | | | | | | | g/content/10.1101/202 |
| | GTKVEIK | | | | | | | | | 0.05.12.091298v1) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| CV47 | Ab | SARS-CoV2 | SARS- | | SARS-CoV2 | S; non- | B-cells; SARS-CoV2 | 742 | QVQLVQSGAEVKKPGASVKVSCKASGY | 1821 |
| | | | CoV1 | | | RBD | Human Patient | | TFTSYGISWVRQAPGQGLEWMGWISA | |
| | | | | | | | | | YSGNTNYAQKLQGRVTMTTDTSTSTAY | |
| | | | | | | | | | MEVRSLRSDDTAVYYCARVGLWWLGH | |
| | | | | | | | | | PDVFDIWGQGTMVTVSS | |
| CV48 | Ab | SARS-CoV2 | | | SARS-CoV2 | S; non- | B-cells; SARS-CoV2 | 743 | QVQLVQSGAEVKKPGSSVKVSCKASGG | 1822 |
| | | | | | | RBD | Human Patient | | TFSSYTINWVRQAPGQGLEWMGRIIPIL | |
| | | | | | | | | | GIADYAQKFQGRVTITADKSTSTAYMEL | |
| | | | | | | | | | SSLRSEDTAVYYCARDLVEDTAMVTGAA | |
| | | | | | | | | | AGTWGQGTLVTVSS | |
| CV5 | Ab | SARS-CoV2 | SARS- | | SARS-CoV2 | S; RBD | B-cells; SARS-CoV2 | 744 | QVQLVQSGAEVKKPGASVKVTCKASGY | 1823 |
| | | | CoV1 | | | | Human Patient | | TFTSYYLHWVRQAPGQGLEWMGIINPS | |
| | | | | | | | | | GGTTSYAQKFQGRVTMTRDTSTSTVYM | |
| | | | | | | | | | ELSSLRSEDTAVYYCARAGRRYSSSDDGA | |
| | | | | | | | | | FDIWGQGTMVTVSS | |
| CV50 | Ab | SARS-CoV2 | | | SARS-CoV2 | S; non- | B-cells; SARS-CoV2 | 745 | QVQLVESGGGVVQPGRSLRLSCAASGF | 1824 |
| | | | | | | RBC | Human Patient | | TFSSYGMHWVRQAPGKGLEWVAVIWY | |
| | | | | | | | | | DGSNKYYADSVKGRFTISRDNSKNTLYL | |
| | | | | | | | | | QMNSLRAEDTAVYYCARDIMFGDDWL | |
| | | | | | | | | | QKQPDYWGQGTLVTVSS | |
| CV7 | Ab | SARS-CoV2 | SARS- | | SARS-CoV2 | S; non- | B-cells; SARS-CoV2 | 746 | QVQLVESGGGVVQPGRSLRLSCAASGF | 1825 |
| | | | CoV1 | | | RBD | Human Patient | | TFSSYAMHWVRQAPGKGLEWVAVISY | |
| | | | | | | | | | DGSNKYYADSVKGRFTISRDNSKNTLYL | |
| | | | | | | | | | QMNSLRAEDTAVYYCARSISGSYLGAFD | |
| | | | | | | | | | YWGQGTLVTVSS | |
| CV8 | Ab | SARS-CoV1, | | | SARS-CoV2 | S; non- | B-cells; SARS-CoV2 | 747 | QVQLVQSGAEVKKPGASVKVSCKASGY | 1826 |
| | | SARS-CoV2 | | | | RBD | Human Patient | | TFTSYGISWVRQAPGQGLEWMGWISA | |
| | | | | | | | | | YNGNTNYAQKLQGRVTMTTDTSTSTAY | |
| | | | | | | | | | MELRSLRSDDTAVYYCARLVPTWASYYD | |
| | | | | | | | | | FWSGYPGGYGMDVWGQGTTVTVSS | |
| CV9 | Ab | SARS-CoV1, | | | SARS-CoV2 | S; non- | B-cells; SARS-CoV2 | 748 | QLQLQESGPGLVKPSETLSLTCTVSGGSI | 1827 |
| | | SARS-CoV2 | | | | RBD | Human Patient | | SSSSYYWGWIRQPPGKGLEWIGSIYYSG | |
| | | | | | | | | | STYYNPSLKSRVTISVDTSKNQFSLKLSSV | |
| | | | | | | | | | TAADTAVYYCATHIVVVTATPNWYFDL | |
| | | | | | | | | | WGRGTLVTVSS | |
| EY6A | Ab | SARS-CoV2 | | SARS-CoV2 | | S; RBD | B-cells; SARS-CoV2 | 749 | EVQLVESGGGVVQPGRSLRLSCAASAFT | 1828 |
| | | | | | | | Human Patient | | FSSYDMHWVRQAPGKGLEWVAVISYD | |
| | | | | | | | | | GSNKYYADSVKGRFTISRDNSKNTLYLQ | |
| | | | | | | | | | MNSLRAEDTAVYYCAKDGGKLWVYYFD | |
| | | | | | | | | | YWGQGTLVTVSS | |
| Fab 2-4 | Ab | SARS-CoV2 | | SARS-CoV2 | | S; RBD | B-cells; SARS-CoV2 | 750 | QVQLVQSGAEVKKPGASVKVSCKASGY | 1829 |
| | | | | | | | Human Patient | | TFTGYYMHWVRQAPGQGLEWMGWI | |
| | | | | | | | | | NPNSGGTNYTQMFQGRVTMTRDTSIST | |
| | | | | | | | | | AYMEVSRLRSDDTAVYYCARDRSWAVV | |
| | | | | | | | | | YYYMDVWGKGTTVTVSS | |
| FnC1t1p2 | Ab | SARS-CoV2 | | SARS-CoV2 | | S; S1 non- | B-cells; SARS-CoV2 | 751 | QVQLVQSGAEVKKPGASVKVSCKASGY | 1830 |
| A5 | | | | | | RBD | Human Patient | | TFTSYDINWVRQATGQGLEWMGWMN | |
| | | | | | | | | | PNSGNTGYAQKFQGRVTMTRNTSISTA | |
| | | | | | | | | | YMELSSLRSEDTAVYYCARATTDCSSTSC | |
| | | | | | | | | | WSLDFWSGYYTGGREKIFDWGQGTLVT | |
| | | | | | | | | | VSS | |
| FnC1t2p1 | Ab | SARS-CoV2 | | SARS-CoV2 | | S; RBD | B-cells; SARS-CoV2 | 752 | QVQLVQSGSELKKPGASVKVSCKASGYT | 1831 |
| D4 | | | | | | | Human Patient | | FLRFAMNWLRQAPGQGLEWMGWIDT | |
| | | | | | | | | | NTGTPTYAQGFTGRFVFSLDTSVSTAYL | |
| | | | | | | | | | QISSLKAEDTAVYYCARSLRGANLVPWG | |
| | | | | | | | | | QGTLVTVSS | |
| FnC1t2p1 | Ab | SARS-CoV2 | | SARS-CoV2 | | S; RBD | B-cells; SARS-CoV2 | 753 | QVQLVQSGSELKKPGASVKVSCKASGYT | 1832 |
| G5 | | | | | | | Human Patient | | FLRFAMNWLRQAPGQGLEWMGWIDT | |
| | | | | | | | | | NTGTPTYAQGFTGRFVFSLDTSVSTAYL | |
| | | | | | | | | | QISSLKAEDTAVYYCARSLRGANLVPWG | |
| | | | | | | | | | QGTLVTVSS | |
| H014 | Ab | SARS-CoV1, | | SARS-CoV1, | | S; RBD | Immunised | | ND | |
| | | SARS-CoV2 | | SARS-CoV2 | | | Humanised (hACE2) | | | |
| | | | | | | | Mouse | | | |
| H11-D4 | Nb | SARS-CoV2 | | SARS-CoV2 | | S; RBD | Phage Display | 754 | QVQLVESGGGLMQAGGSLRLSCAVSGR | |
| | | | | | | | Library (Nanobody, | | TFSTAAMGWFRQAPGKEREFVAAIRWS | |
| | | | | | | | non-immune) | | GGSAYYADSVKGRFTISRDKAKNTVYLQ | |
| | | | | | | | | | MNSLKYEDTAVYYCARTENVRSLLSDYA | |
| | | | | | | | | | TWPYDYWGQGTQVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| CV47 | DIQMTQSPSSLSASVGDRVTITCRASQ | IGHV1-18 | IGHJ3 | IGKV1-17 | IGKJ4 | 2848 | ARVGLWWL | 4050 | LQHNSYPL | Emilie Seydoux et al., |
| | GIRNDLGWYQQKPGKAPKRLIYAASSL | (Human) | (Human) | (Human) | (Human) | | GHPDVFDI | | T | 2020 |
| | QSGVPSRFSGSGSGTEFTLTISSLQPED | | | | | | | | | (https://www.biorxiv.or |
| | FATYYCLQHNSYPLTFGGGTKVEIK | | | | | | | | | g/content/10.1101/202 |
| | | | | | | | | | | 0.05.12.091298v1) |
| CV48 | DVVMTQSPLSLPVTLGQPASISCRSSQ | IGHV1-69 | IGHJ4 | IGKV2-30 | IGKJ1 | 2849 | ARDLVEDTA | 4051 | MQGTHW | Emilie Seydoux et al., |
| | SLVYSDGNTYLNWFQQRPGQSPRRLI | (Human) | (Human) | (Human) | (Human) | | MVTGAAAGT | | PPT | 2020 |
| | YKVSNRDSGVPDKFSGSGSGTDFTLKI | | | | | | | | | (https://www.biorxiv.or |
| | SRVEAEDVGVYYCMQGTHWPPTFGQ | | | | | | | | | g/content/10.1101/202 |
| | GTKVEIK | | | | | | | | | 0.05.12.091298v1) |
| CV5 | DIVMTQSPDSLAVSLGERATINCKSSQ | IGHV1-46 | IGHJ3 | IGKV4-1 | IGKJ2 | 2850 | ARAGRRYSSS | 4052 | QQYYITPYT | Emilie Seydoux et al., |
| | NVLYSSNNKNYLAWYQQKPGQPPKLL | (Human) | (Human) | (Human) | (Human) | | DDGAFDI | | | 2020 |
| | IYWASTRESGVPDRFSGSGSGTDFTLTI | | | | | | | | | (https://www.biorxiv.or |
| | SSLQAEDVAVYYCQQYYITPYTFGQGT | | | | | | | | | g/content/10.1101/202 |
| | KLEIK | | | | | | | | | 0.05.12.091298v1) |
| CV50 | SYELTQPPSVSVSPGQTARITCSGDALP | IGHV3-33 | IGHJ4 | IGLV3-10 | IGLJ1 | 2851 | ARDIMFGDD | 4053 | YSTDSSGN | Emilie Seydoux et al., |
| | KKYAYWYQQKSGQAPVLVIYEDSKRP | (Human) | (Human) | (Human) | (Human) | | WLQKQPDY | | LYV | 2020 |
| | SGIPERFSGSSSGTMATLTISGAQVED | | | | | | | | | (https://www.biorxiv.or |
| | EADYYCYSTDSSGNLYVFGTGTKVTVL | | | | | | | | | g/content/10.1101/202 |
| | | | | | | | | | | 0.05.12.091298v1) |
| CV7 | DIQMTQSPSTLSASVGDRVTITCRASQ | IGHV3-30 | IGHJ4 | IGKV1-5 | IGKJ2 | 2852 | ARSISGSYLGA | 4054 | QQYNSYT | Emilie Seydoux et al., |
| | SISSWLAWYQQKPGKAPKLLIYKASSL | (Human) | (Human) | (Human) | (Human) | | FDY | | | 2020 |
| | ESGVPSRFSGSGSGTEFTLTISSLQPDD | | | | | | | | | (https://www.biorxiv.or |
| | FATYYCQQYNSYTFGQGTKLEIK | | | | | | | | | g/content/10.1101/202 |
| | | | | | | | | | | 0.05.12.091298v1) |
| CV8 | EIVLTQSPGTLSLSPGERATLSCRASQS | IGHV1-18 | IGHJ6 | IGKV3-20 | IGKJ5 | 2853 | ARLVPTWASY | 4055 | QQYGSSP | Emilie Seydoux et al., |
| | VSSSYLAWYQQKPGQAPRLLIYGASSR | (Human) | (Human) | (Human) | (Human) | | YDFWSGYPG | | GT | 2020 |
| | ATGIPDRFSGSGSGTDFTLTISRLEPED | | | | | | GYGMDV | | | (https://www.biorxiv.or |
| | FAVYYCQQYGSSPGTFGQGTRLEIK | | | | | | | | | g/content/10.1101/202 |
| | | | | | | | | | | 0.05.12.091298v1) |
| CV9 | QSALTQPASVSGSPGQSITISCTGTSSD | IGHV4-39 | IGHJ2 | IGLV2-14 | IGLJ3 | 2854 | ATHIVVVTAT | 4056 | SSYTSISTW | Emilie Seydoux et al., |
| | VGGYNYVSWYQQHPGKAPKLMIYDV | (Human) | (Human) | (Human) | (Human) | | PNWYFDL | | V | 2020 |
| | SNRPSGVSNRFSGSKSGNTASLTISGL | | | | | | | | | (https://www.biorxiv.or |
| | QAEDEADYYCSSYTSISTWVFGGGTKL | | | | | | | | | g/content/10.1101/202 |
| | TVL | | | | | | | | | 0.05.12.091298v1) |
| EY6A | DIQMTQSPSSLSASVGDRVTITCRASQ | IGHV3-30- | IGHJ4 | IGKV1-39 | IGKJ4 | 2855 | AKDGGKLWV | 4057 | QQSYSTLA | Daming Zhou et al., |
| | SISSYLNWYQQKPGKAPKLLIYAASSLQ | 3 | (Human) | (Human) | (Human) | | YYFDY | | LT | 2020 |
| | SGVPSRFSGSGSGTDFTLTISSLQPEDF | (Human) | | | | | | | | (https://www.biorxiv.or |
| | ATYYCQQSYSTLALTFGGGTKVEIK | | | | | | | | | g/content/10.1101/202 |
| | | | | | | | | | | 0.06.12.148387v1) |
| Fab 2-4 | QSALTQPPSASGSPGQSVTISCTGTSS | IGHV1-2 | IGHJ6 | IGLV2-8 | IGLJ3 | 2856 | ARDRSWAVV | 4058 | SSYAGSNN | David Ho et al., 2020 |
| | DVGGYNYVSWYQQHPGKAPKLMIYE | (Human) | (Human) | (Human) | (Human) | | YYYMDV | | LV | (https://www.biorxiv.or |
| | VSKRPSGVPDRFSGSKSGNTASLTVSG | | | | | | | | | g/content/10.1101/202 |
| | LQAEDEADYYCSSYAGSNNLVFGGGT | | | | | | | | | 0.06.17.153486v1) |
| | KLTVL | | | | | | | | | |
| FnC1t1p2 | EIVLTQSPGTLSLSPGERATLSCRASQS | IGHV1-8 | IGHJ4 | IGKV3-20 | IGKJ1 | 2857 | ARATTDCSST | 4059 | QQYGSSP | Christoph Kreer et al., |
| A5 | VSSSYLAWYQQKPGQAPRLLIYGASSR | (Human) | (Human) | (Human) | (Human) | | SCWSLDFWS | | GT | 2020 |
| | ATGIPDRFSGSGSGTDFTLTISRLEPED | | | | | | GYYTGGREKI | | | (https://doi.org/10.101 |
| | FAVYYCQQYGSSPGTFGQGTKVEIK | | | | | | FD | | | 6/j.cell.2020.06.044) |
| FnC1t2p1 | DIQMTQSPSSLSASVGDRVTITCQASQ | IGHV7-4- | IGHJ5 | IGKV1-33 | IGKJ4 | 2858 | ARSLRGANLV | 4060 | QQYDNLPL | (https://doi.org/10.101 |
| D4 | DVSNYLNWYQQQPGKAPKLLIYDAFN | 1 (Human) | (Human) | (Human) | (Human) | | P | | T | Christoph Kreer et al., |
| | LETGVPSRFSGSGSGTDFTFTISSLQPE | | | | | | | | | 2020 |
| | DIATYYCQQYDNLPLTFGGGTKVEIK | | | | | | | | | 6/j.cell.2020.06.044) |
| FnC1t2p1 | DIQMTQSPSSLSASVGDRVTITCQASQ | IGHV7-4- | IGHJ5 | IGKV1-33 | IGKJ4 | 2859 | ARSLRGANLV | 4061 | QQYDNLPL | Christoph Kreer et al., |
| G5 | DVSNYLNWYQQQPGKAPKLLIYDAFN | 1 (Human) | (Human) | (Human) | (Human) | | P | | T | 2020 |
| | LETGVPSRFSGSGSGTDFTFTISSLQPE | | | | | | | | | (https://doi.org/10.101 |
| | DIATYYCQQYDNLPLTFGGGTKVEIK | | | | | | | | | 6/j.cell.2020.06.044) |
| H014 | ND | ND | ND | ND | ND | | ND | | ND | Zhe Lv et al., 2020 |
| | | | | | | | | | | (https://www.biorxiv.or |
| | | | | | | | | | | g/content/10.1101/202 |
| | | | | | | | | | | 0.06.02.129098v2) |
| H11-D4 | N/A | IGHV3-3 | (Alpaca) | N/A | N/A | 2860 | ARTENVRSLL | | N/A | Jiangdong Huo et al., |
| | | (Alpaca) | IGHJ4 | | | | SDYATWPYDY | | | 2020 |
| | | | | | | | | | | (https://www.nature.co |
| | | | | | | | | | | m/articles/s41594-020- |
| | | | | | | | | | | 0469-6) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| H11-H4 | Nb | SARS-CoV2 | | SARS-CoV2 | | S; RBD | Phage Display | 755 | QVQLVESGGGLMQAGGSLRLSCAVSGR | |
| | | | | | | | Library (Nanobody, | | TFSTAAMGWFRQAPGKEREFVAAIRWS | |
| | | | | | | | non-immune) | | GGSAYYADSVKGRFTISRDKAKNTVYLQ | |
| | | | | | | | | | MNSLKYEDTAVYYCAQTHYVSYLLSDYA | |
| | | | | | | | | | TWPYDYWGQGTQVTVSS | |
| H4 | Ab | SARS-CoV2 | | SARS-CoV2 | | S; RBD | B-cells; SARS-CoV2 | 756 | QVQLVQSGAEVKKPGASVKVSCKASGY | 1833 |
| | | | | | | | Human Patient | | TFTGYYMHWVRQAPGQGLEWMGRIN | |
| | | | | | | | | | PNSGGTNYAQKFQGRVTMTRDTSISTA | |
| | | | | | | | | | YMELSRLRSDDTAVYYCARVPYCSSTSCH | |
| | | | | | | | | | RDWYFDLWGRGTLVTVSS | |
| HbnC2t1 | Ab | SARS-CoV2 | | SARS-CoV2 | | S; RBD | B-cells; SARS-CoV2 | 757 | QVQLVESGGGVVQPGRSLRLSCAASGF | 1834 |
| p2_D9 | | | | | | | Human Patient | | TFSSYGMHWVRQAPGKGLEWVAVIWY | |
| | | | | | | | | | DGRNKYYVDSVKGRFTISRDNSKNTLYL | |
| | | | | | | | | | QISSLRAEDTAVYYCARAARRPVVTDTM | |
| | | | | | | | | | AYYMDVWGKGTTVTVSS | |
| HbnC3t1 | Ab | SARS-CoV2 | | SARS-CoV2 | | S; RBD | B-cells; SARS-CoV2 | 758 | QMQLVQSGPEVKKPGTSVKVSCKASGF | 1835 |
| p1_C6 | | | | | | | Human Patient | | TFTSSAVQWVRQARGQRLEWIGWIVV | |
| | | | | | | | | | GSGNTNYAQKFQERVTITRDMSTSTAY | |
| | | | | | | | | | MELSSLRSEDTAVYYCAAPHCSSTICYDG | |
| | | | | | | | | | FDIWGQGTMVTVSS | |
| HbnC3t1 | Ab | SARS-CoV2 | | SARS-CoV2 | | S; RBD | B-cells; SARS-CoV2 | 759 | QVQLVESGGGVVQPGRSLRLSCAATGF | 1836 |
| p1_F4 | | | | | | | Human Patient | | TFRRYGMHWVRQAPGKGLEWVAGILF | |
| | | | | | | | | | DGSNKYYVDSVKGRFTISRDSSRNTLYLQ | |
| | | | | | | | | | LNSLRREDTAVYYCAKGGDYEWELLES | |
| | | | | | | | | | WGQGTLVTVSS | |
| HbnC3t1 | Ab | SARS-CoV2 | | SARS-CoV2 | | S; RBD | B-cells; SARS-CoV2 | 760 | EVQLVESGGGLVQPGGSLRLSCAASGFT | 1837 |
| p1_G4 | | | | | | | Human Patient | | VSSNYMSWVRQAPGKGLEWVSVIYSG | |
| | | | | | | | | | GSTFYADSVKGRFTISRDNSKNTLYLQM | |
| | | | | | | | | | NSLRAEDTAVYYCARDFGDFFFDYWGQ | |
| | | | | | | | | | GTLVTVSS | |
| HbnC3t1 | Ab | SARS-CoV2 | | SARS-CoV2 | | S; RBD | B-cells; SARS-CoV2 | 761 | EVQLVESGGGLVQPGGSLRLSCAASGFI | 1838 |
| p2_B10 | | | | | | | Human Patient | | VSSNYMSWVRQAPGKGLEWVSVIYSG | |
| | | | | | | | | | GSTFYADSVKGRFTISRDNSKNTLYLQM | |
| | | | | | | | | | NSLRAEDTAVYYCARDYGDYFFDYWGQ | |
| | | | | | | | | | GTLVTVSS | |
| HbnC3t1 | Ab | SARS-CoV2 | | SARS-CoV2 | | S; RBD | B-cells; SARS-CoV2 | 762 | QMQLVQSGPEVKKPGTSVKVSCKASGF | 1839 |
| p2_C6 | | | | | | | Human Patient | | TFSSSAVQWVRQARGQRLEWIGWIVV | |
| | | | | | | | | | GSGNTNYAQKFQGRVTITRDMSTRTAY | |
| | | | | | | | | | MELSSLRSEDTAMYYCAAPYCSSTRCYD | |
| | | | | | | | | | AFDIWGQGTMVTVSS | |
| HbnC4t1 | Ab | SARS-CoV2 | | SARS-CoV2 | | S; RBD | B-cells; SARS-CoV2 | 763 | EVQLVESGGGLVQPGRSLRLSCAASGFT | 1840 |
| p1_D5 | | | | | | | Human Patient | | FDDYAMHWVRQAPGKGLEWVSGISW | |
| | | | | | | | | | NSGSIGYADSVKGRFTISRDNAKNSLYLQ | |
| | | | | | | | | | MNSLRAEDTGLYYCAKDINYDSGGYHK | |
| | | | | | | | | | NYFDYWGQGTLVTVSS | |
| Ju et | Ab | SARS-CoV2 | | SARS-CoV2 | SARS-CoV2 | S; Various | B-cells; SARS-CoV2 | | 205 | |
| al., | | | | | | | Human Patient | | | |
| 2020 | | | | | | | | | | |
| Kim et | Ab | SARS-CoV2 | | SARS-CoV2 | | S; RBD | B-cells; SARS-CoV2 | | Various | |
| al., | | | | | | | Human Patient | | | |
| 2020 | | | | | | | | | | |
| LR1 | Nb | SARS-CoV2 | | | | S; RBD | Phage Display | 764 | QVQLVESGGGSVQAGGSLRLSCAASGSI | |
| | | | | | | | Library (Nanobody, | | SSITYLGWFRQAPGKEREGVAALITDSG | |
| | | | | | | | non-immune) | | RTYYADSVKGRFTVSLDNAKNTVYLQM | |
| | | | | | | | | | NSLKPEDTALYYCAAAEWGYEWPLYYAS | |
| | | | | | | | | | SWYWGQGTQVTVSS | |
| LR11 | Nb | SARS-CoV2 | | | | S; RBD | Phage Display | 765 | QVQLVESGGGSVQAGGSLRLSCAASGSI | |
| | | | | | | | Library (Nanobody, | | SSITYLGWFRQAPGKEREGVAALITDSG | |
| | | | | | | | non-immune) | | RTYYADSVKGRFTVSLDNAKNTVYLQM | |
| | | | | | | | | | NSLKPEDTALYYCAAAEWGYEWPLYYAS | |
| | | | | | | | | | SWYWGQGTQVTVSS | |
| LR15 | Nb | SARS-CoV2 | | | | S; RBD | Phage Display | 766 | QVQLVESGGGSVQAGGSLRLSCAASGSI | |
| | | | | | | | Library (Nanobody, | | SSITYLGWFRQAPGKEREGVAALITDSG | |
| | | | | | | | non-immune) | | RTYYADSVKGRFTVSLDNAKNTVYLQM | |
| | | | | | | | | | NSLKPEDTALYYCAAAEWGYEWPLYYAS | |
| | | | | | | | | | SWYWGQGTQVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| H11-H4 | N/A | IGHV3-3 | IGHJ4 | N/A | N/A | 2861 | AQTHYVSYLL | | N/A | Jiangdong Huo et al., |
| | | (Alpaca) | (Alpaca) | | | | SDYATWPYDY | | | 2020 |
| | | | | | | | | | | (https://www.nature.co |
| | | | | | | | | | | m/articles/s41594-020- |
| | | | | | | | | | | 0469-6) |
| H4 | DIQMTQSPLSLPVTPGEPASISCRSSQS | IGHV1-2 | IGHJ2 | IGKV2-40 | IGKJ4 | 2862 | ARVPYCSSTSC | 4062 | MQRIEFPL | Yan Wu et al., 2020 |
| | LLDSDDGNTYLDWYLQKPGQSPQLLIY | (Human) | (Human) | (Human) | (Human) | | HRDWYFDL | | T | (https://science.science |
| | TLSYRASGVPDRFSGSGSGTDFTLKISR | | | | | | | | | mag.org/content/early/ |
| | VEAEDVGVYYCMQRIEFPLTFGGGTK | | | | | | | | | 2020/05/12/science.abc |
| | VEIK | | | | | | | | | 2241) |
| HbnC2t1 | EIVLTQSPATLSLSPGERATLSCRASLSL | IGHV3-33 | IGHJ6 | IGKV3-11 | IGKJ1 | 2863 | ARAARRPVVT | 4063 | QQRSNWP | Christoph Kreer et al., |
| p2_D9 | SSYLAWYQQKPGQAPRLLIYDASNRA | (Human) | (Human) | (Human) | (Human) | | DTMAYYMDV | | PTWT | 2020 |
| | TGIPARFSGSGSGTDFTLTISSLEPEDFA | | | | | | | | | (https://doi.org/10.101 |
| | VYYCQQRSNWPPTWTFGQGTKAEIK | | | | | | | | | 6/j.cell.2020.06.044) |
| HbnC3t1 | EIVLTQSPGTLSLSPGERATLSCRASQS | IGHV1-58 | IGHJ3 | IGKV3-20 | IGKJ1 | 2864 | AAPHCSSTICY | 4064 | QQYGSSP | Christoph Kreer et al., |
| p1_C6 | VSSSYLAWYQQKPGQAPRLLIYGASSR | (Human) | (Human) | (Human) | (Human) | | DGFDI | | WT | 2020 |
| | ATGIPDRFSGSGSGTDFTLTISRVEPED | | | | | | | | | (https://doi.org/10.101 |
| | FAVYYCQQYGSSPWTFGQGTKVEIK | | | | | | | | | 6/j.cell.2020.06.044) |
| HbnC3t1 | DIQMTQSPSTVSASVGDRVTITCRASQ | IGHV3-30 | IGHJ5 | IGKV1-5 | IGKJ4 | 2865 | AKGGDYEWE | 4065 | QHYHSFP | (https://doi.org/10.101 |
| p1_F4 | SIDNWLAWYQEKPGKAPKVLIYKASSL | (Human) | (Human) | (Human) | (Human) | | LLES | | LT | Christoph Kreer et al., |
| | ESGVPSRFSGRGSGTEFTLTISSLQPGD | | | | | | | | | 2020 |
| | FATYYCQHYHSFPLTFGGGTKVDIK | | | | | | | | | 6/j.cell.2020.06.044) |
| HbnC3t1 | EIVLTQSPGTLSLSPGERATLSCRASQS | IGHV3-66 | IGHJ4 | IGKV3-20 | IGKJ1 | 2866 | ARDFGDFFFD | 4066 | QQYGSSP | Christoph Kreer et al., |
| p1_G4 | VSSYLAWYQQKPGQAPRLLIYGVSSRA | (Human) | (Human) | (Human) | (Human) | | Y | | RT | 2020 |
| | TGIPDRFSGSGSGTDFTLTISRLEPEDF | | | | | | | | | (https://doi.org/10.101 |
| | AVYYCQQYGSSPRTFGQGTKVEIK | | | | | | | | | 6/j.cell.2020.06.044) |
| HbnC3t1 | EIVLTQSPGTLSLSPGERATLSCRASQS | IGHV3-66 | IGHJ4 | IGKV3-20 | IGKJ2 | 2867 | ARDYGDYFFD | 4067 | QQYGSSP | (https://doi.org/10.101 |
| p2_B10 | VSSYLAWYQQKPGQAPRLLISGASSRA | (Human) | (Human) | (Human) | (Human) | | Y | | RT | Christoph Kreer et al., |
| | AGIPDRFSGSGSGTDFTLTINRLEPEDF | | | | | | | | | 2020 |
| | AVYYCQQYGSSPRTFGQGTKLEIK | | | | | | | | | 6/j.cell.2020.06.044) |
| HbnC3t1 | EIVLTQSPGTLSLSPGERATLSCRASQS | IGHV1-58 | IGHJ3 | IGKV3-20 | IGKJ1 | 2868 | AAPYCSSTRC | 4068 | QQYGRSP | Christoph Kreer et al., |
| p2_C6 | VSSSYLAWYQQKPGQAPRLLIYGASSR | (Human) | (Human) | (Human) | (Human) | | YDAFDI | | WT | 2020 |
| | ATGIPDRFSGSGSGTDFTLTISRLEPED | | | | | | | | | (https://doi.org/10.101 |
| | FAVYYCQQYGRSPWTFGQGTKVEI | | | | | | | | | 6/j.cell.2020.06.044) |
| HbnC4t1 | DIQMTQSPSSLSASVGDRATITCRASQ | IGHV3-9 | IGHJ4 | IGKV1-39 | IGKJ4 | 2869 | AKDINYDSGG | 4069 | QQSYSNP | Christoph Kreer et al., |
| p1_D5 | SISSYLNWYQEKPGKAPKLLIYAASSLQ | (Human) | (Human) | (Human) | (Human) | | YHKNYFDY | | LT | 2020 |
| | SGVPSRFSGSGSGTDFTLTISSLQPEDF | | | | | | | | | (https://doi.org/10.101 |
| | ATYYCQQSYSNPLTFGGGTKVEIK | | | | | | | | | 6/j.cell.2020.06.044) |
| Ju et | 205 | Various | Various | Various | Various | | Various | | Various | Bin Ju et al., 2020 |
| al., | | | | | | | | | | (https://www.nature.co |
| 2020 | | | | | | | | | | m/articles/s41586-020- |
| | | | | | | | | | | 2380-z) |
| Kim et | Various | Various | Various | Various | Various | | Various | | Various | Sang II Kim et al., 2020 |
| al., | | | | | | | | | | (https://www.biorxiv.or |
| 2020 | | | | | | | | | | g/content/10.1101/202 |
| | | | | | | | | | | 0.06.26.174557v2) |
| LR1 | N/A | IGHV3-3 | IGHJ4 | N/A | N/A | 2870 | AAAEWGYE | | N/A | Tingting Li et al., 2020 |
| | | (Alpaca) | (Alpaca) | | | | WPLYYASSW | | | (https://www.biorxiv.or |
| | | | | | | | Y | | | g/content/10.1101/202 |
| | | | | | | | | | | 0.06.09.143438v1) |
| LR11 | N/A | IGHV3-3 | IGHJ4 | N/A | N/A | 2871 | AAAYWGWD | | N/A | Tingting Li et al., 2020 |
| | | (Alpaca) | (Alpaca) | | | | WPLNSQDYW | | | (https://www.biorxiv.or |
| | | | | | | | Y | | | g/content/10.1101/202 |
| | | | | | | | | | | 0.06.09.143438v1) |
| LR15 | N/A | IGHV3-3 | IGHJ4 | N/A | N/A | 2872 | AAADWGYN | | N/A | Tingting Li et al., 2020 |
| | | (Alpaca) | (Alpaca) | | | | WPLIREEYEY | | | (https://www.biorxiv.or |
| | | | | | | | | | | g/content/10.1101/202 |
| | | | | | | | | | | 0.06.09.143438v1) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| LR16 | Nb | SARS-CoV2 | | | | S; RBD | Phage Display | 767 | QVQLVESGGGSVQAGGSLRLSCAASGSI | |
| | | | | | | | Library (Nanobody, | | SSITYLGWFRQAPGKEREGVAALITDSG | |
| | | | | | | | non-immune) | | RTYYADSVKGRFTVSLDNAKNTVYLQM | |
| | | | | | | | | | NSLKPEDTALYYCAAAEWGYEWPLYYAS | |
| | | | | | | | | | SWYWGQGTQVTVSS | |
| LR2 | Nb | SARS-CoV2 | | | | S; RBD | Phage Display | 768 | QVQLVESGGGSVQAGGSLRLSCAASGSI | |
| | | | | | | | Library (Nanobody, | | SSITYLGWFRQAPGKEREGVAALITDSG | |
| | | | | | | | non-immune) | | RTYYADSVKGRFTVSLDNAKNTVYLQM | |
| | | | | | | | | | NSLKPEDTALYYCAAAEWGYEWPLYYAS | |
| | | | | | | | | | SWYWGQGTQVTVSS | |
| LR3 | Nb | SARS-CoV2 | | | | S; RBD | Phage Display | 769 | QVQLVESGGGSVQAGGSLRLSCAASGSI | |
| | | | | | | | Library (Nanobody, | | SSITYLGWFRQAPGKEREGVAALITDSG | |
| | | | | | | | non-immune) | | RTYYADSVKGRFTVSLDNAKNTVYLQM | |
| | | | | | | | | | NSLKPEDTALYYCAAAEWGYEWPLYYAS | |
| | | | | | | | | | SWYWGQGTQVTVSS | |
| LR5 | Nb | SARS-CoV2 | | | | S; RBD | Phage Display | 770 | QVQLVESGGGSVQAGGSLRLSCAASGSI | |
| | | | | | | | Library (Nanobody, | | SSITYLGWFRQAPGKEREGVAALITDSG | |
| | | | | | | | non-immune) | | RTYYADSVKGRFTVSLDNAKNTVYLQM | |
| | | | | | | | | | NSLKPEDTALYYCAAAEWGYEWPLYYAS | |
| | | | | | | | | | SWYWGQGTQVTVSS | |
| LR6 | Nb | SARS-CoV2 | | | | S; RBD | Phage Display | 771 | QVQLVESGGGSVQAGGSLRLSCAASGSI | |
| | | | | | | | Library (Nanobody, | | SSITYLGWFRQAPGKEREGVAALITDSG | |
| | | | | | | | non-immune) | | RTYYADSVKGRFTVSLDNAKNTVYLQM | |
| | | | | | | | | | NSLKPEDTALYYCAAAEWGYEWPLYYAS | |
| | | | | | | | | | SWYWGQGTQVTVSS | |
| LR7 | Nb | SARS-CoV2 | | | | S; RBD | Phage Display | 772 | QVQLVESGGGSVQAGGSLRLSCAASGSI | |
| | | | | | | | Library (Nanobody, | | SSITYLGWFRQAPGKEREGVAALITDSG | |
| | | | | | | | non-immune) | | RTYYADSVKGRFTVSLDNAKNTVYLQM | |
| | | | | | | | | | NSLKPEDTALYYCAAAEWGYEWPLYYAS | |
| | | | | | | | | | SWYWGQGTQVTVSS | |
| LR8 | Nb | SARS-CoV2 | | | | S; RBD | Phage Display | 773 | QVQLVESGGGSVQAGGSLRLSCAASGSI | |
| | | | | | | | Library (Nanobody, | | SSITYLGWFRQAPGKEREGVAALITDSG | |
| | | | | | | | non-immune) | | RTYYADSVKGRFTVSLDNAKNTVYLQM | |
| | | | | | | | | | NSLKPEDTALYYCAAAEWGYEWPLYYAS | |
| | | | | | | | | | SWYWGQGTQVTVSS | |
| mAb-1 | Ab | SARS-CoV1, | 229E, | | | S; Unk | B-cells; SARS-CoV1 | 774 | QVQLVESGGGSVQAGGSLRLSCAASGSI | 1841 |
| | | SARS-CoV2 | HKU1, | | | | Human Patient | | SSITYLGWFRQAPGKEREGVAALITDSG | |
| | | | NL63, | | | | | | RTYYADSVKGRFTVSLDNAKNTVYLQM | |
| | | | OC43 | | | | | | NSLKPEDTALYYCAAAEWGYEWPLYYAS | |
| | | | | | | | | | SWYWGQGTQVTVSS | |
| mAb-10 | Ab | SARS-CoV1, | 229E, | | | S; Unk | B-cells; SARS-CoV1 | 775 | QVQLVESGGGSVQAGGSLRLSCAASGSI | 1842 |
| | | SARS-CoV2 | HKU1, | | | | Human Patient | | SSITYLGWFRQAPGKEREGVAALITDSG | |
| | | | NL63, | | | | | | RTYYADSVKGRFTVSLDNAKNTVYLQM | |
| | | | OC43 | | | | | | NSLKPEDTALYYCAAAEWGYEWPLYYAS | |
| | | | | | | | | | SWYWGQGTQVTVSS | |
| mAb-100 | Ab | SARS-CoV2 | 229E, | | | S; Unk | B-cells; SARS-CoV1 | 776 | QVQLVESGGGSVQAGGSLRLSCAASGSI | 1843 |
| | | (weak) | HKU1, | | | | Human Patient | | SSITYLGWFRQAPGKEREGVAALITDSG | |
| | | | NL63, | | | | | | RTYYADSVKGRFTVSLDNAKNTVYLQM | |
| | | | OC43, | | | | | | NSLKPEDTALYYCAAAEWGYEWPLYYAS | |
| | | | SARS- | | | | | | SWYWGQGTQVTVSS | |
| | | | CoV1 | | | | | | | |
| mAb-101 | Ab | SARS-CoV2 | 229E, | | | S; Unk | B-cells; SARS-CoV1 | 777 | QVQLVESGGGSVQAGGSLRLSCAASGSI | 1844 |
| | | (weak) | HKU1, | | | | Human Patient | | SSITYLGWFRQAPGKEREGVAALITDSG | |
| | | | NL63, | | | | | | RTYYADSVKGRFTVSLDNAKNTVYLQM | |
| | | | OC43, | | | | | | NSLKPEDTALYYCAAAEWGYEWPLYYAS | |
| | | | SARS- | | | | | | SWYWGQGTQVTVSS | |
| | | | CoV1 | | | | | | | |
| mAb-102 | Ab | SARS-CoV2 | 229E, | | | S; Unk | B-cells; SARS-CoV1 | 778 | QVQLVESGGGSVQAGGSLRLSCAASGSI | 1845 |
| | | (weak) | HKU1, | | | | Human Patient | | SSITYLGWFRQAPGKEREGVAALITDSG | |
| | | | NL63, | | | | | | RTYYADSVKGRFTVSLDNAKNTVYLQM | |
| | | | OC43, | | | | | | NSLKPEDTALYYCAAAEWGYEWPLYYAS | |
| | | | SARS- | | | | | | SWYWGQGTQVTVSS | |
| | | | CoV1 | | | | | | | |
| mAb-103 | Ab | SARS-CoV2 | 229E, | | | S; Unk | B-cells; SARS-CoV1 | 779 | QVQLVESGGGSVQAGGSLRLSCAASGSI | 1846 |
| | | (weak) | HKU1, | | | | Human Patient | | SSITYLGWFRQAPGKEREGVAALITDSG | |
| | | | NL63, | | | | | | RTYYADSVKGRFTVSLDNAKNTVYLQM | |
| | | | OC43, | | | | | | NSLKPEDTALYYCAAAEWGYEWPLYYAS | |
| | | | SARS- | | | | | | SWYWGQGTQVTVSS | |
| | | | CoV1 | | | | | | | |
| mAb-104 | Ab | SARS-CoV2 | 229E, | | | S; Unk | B-cells; SARS-CoV1 | 780 | QVQLVESGGGSVQAGGSLRLSCAASGSI | 1847 |
| | | (weak) | HKU1, | | | | Human Patient | | SSITYLGWFRQAPGKEREGVAALITDSG | |
| | | | NL63, | | | | | | RTYYADSVKGRFTVSLDNAKNTVYLQM | |
| | | | OC43, | | | | | | NSLKPEDTALYYCAAAEWGYEWPLYYAS | |
| | | | SARS- | | | | | | SWYWGQGTQVTVSS | |
| | | | CoV1 | | | | | | | |
| mAb-105 | Ab | SARS-CoV2 | 229E, | | | S; Unk | B-cells; SARS-CoV1 | 781 | QVQLVESGGGSVQAGGSLRLSCAASGSI | 1848 |
| | | (weak) | HKU1, | | | | Human Patient | | SSITYLGWFRQAPGKEREGVAALITDSG | |
| | | | NL63, | | | | | | RTYYADSVKGRFTVSLDNAKNTVYLQM | |
| | | | OC43, | | | | | | NSLKPEDTALYYCAAAEWGYEWPLYYAS | |
| | | | SARS- | | | | | | SWYWGQGTQVTVSS | |
| | | | CoV1 | | | | | | | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| LR16 | N/A | IGHV3-3 | IGHJ4 | N/A | N/A | 2873 | AAADWGYNI | | N/A | Tingting Li et al., 2020 |
| | | (Alpaca) | (Alpaca) | | | | PLNITDYWY | | | (https://www.biorxiv.or |
| | | | | | | | | | | g/content/10.1101/202 |
| | | | | | | | | | | 0.06.09.143438v1) |
| LR2 | N/A | IGHV3-3 | IGHJ4 | N/A | N/A | 2874 | AAAMNGYNE | | N/A | Tingting Li et al., 2020 |
| | | (Alpaca) | (Alpaca) | | | | PLYSYDYEY | | | (https://www.biorxiv.or |
| | | | | | | | | | | g/content/10.1101/202 |
| | | | | | | | | | | 0.06.09.143438v1) |
| LR3 | N/A | IGHV3-3 | IGHJ4 | N/A | N/A | 2875 | AAASWGYEW | | N/A | Tingting Li et al., 2020 |
| | | (Alpaca) | (Alpaca) | | | | PLVYDDYWY | | | (https://www.biorxiv.or |
| | | | | | | | | | | g/content/10.1101/202 |
| | | | | | | | | | | 0.06.09.143438v1) |
| LR5 | N/A | IGHV3-3 | IGHJ4 | N/A | N/A | 2876 | AAATWGYH | | N/A | Tingting Li et al., 2020 |
| | | (Alpaca) | (Alpaca) | | | | WPLGAWDY | | | (https://www.biorxiv.or |
| | | | | | | | WY | | | g/content/10.1101/202 |
| | | | | | | | | | | 0.06.09.143438v1) |
| LR6 | N/A | IGHV3-3 | IGHJ4 | N/A | N/A | 2877 | AAATWGYSW | | N/A | Tingting Li et al., 2020 |
| | | (Alpaca) | (Alpaca) | | | | PLEHDEYWY | | | (https://www.biorxiv.or |
| | | | | | | | | | | g/content/10.1101/202 |
| | | | | | | | | | | 0.06.09.143438v1) |
| LR7 | N/A | IGHV3-3 | IGHJ4 | N/A | N/A | 2878 | AAAFHGEQY | | N/A | Tingting Li et al., 2020 |
| | | (Alpaca) | (Alpaca) | | | | PLYTNKYHY | | | (https://www.biorxiv.or |
| | | | | | | | | | | g/content/10.1101/202 |
| | | | | | | | | | | 0.06.09.143438v1) |
| LR8 | N/A | IGHV3-3 | IGHJ4 | N/A | N/A | 2879 | AAANYGANF | | N/A | Tingting Li et al., 2020 |
| | | (Alpaca) | (Alpaca) | | | | PLQANTYFY | | | (https://www.biorxiv.or |
| | | | | | | | | | | g/content/10.1101/202 |
| | | | | | | | | | | 0.06.09.143438v1) |
| mAb-1 | QPVLTQPPSVSGAPGQRITISCTGSSS | IGHV3-21 | IGHJ4 | IGLV1-40 | IGLJ1 | 2880 | ARDFSGHTAV | 4070 | QSYDSSLS | Anna Wec et al., 2020 |
| | NIGAGYDVHWYQQLPGTAPKLLIYGS | (Human) | (Human) | (Human) | (Human) | | AGTGFEY | | VLYV | (https://science.science |
| | SSRPSGVPDRFSGSKSGTSASLAITGLQ | | | | | | | | | mag.org/content/early/ |
| | AEDEADYYCQSYDSSLSVLYVFGTGTK | | | | | | | | | 2020/06/15/science.abc |
| | VTVL | | | | | | | | | 7424) |
| mAb-10 | DIVLTQTPATLSVSPGEGATLSCRASQS | IGHV3-66 | IGHJ4 | IGKV3-15 | IGKJ4 | 2881 | VRASPPGGN | 4071 | QQYNSWP | Anna Wec et al., 2020 |
| | VRSNLAWFQQRPGQVPRLLIYDASTR | (Human) | (Human) | (Human) | (Human) | | TGWPFFED | | PLT | (https://science.science |
| | ATGVPARFTGSGSGTYFTLTISSLQSED | | | | | | | | | mag.org/content/early/ |
| | FAVYYCQQYNSWPPLTFGGGTKLEIK | | | | | | | | | 2020/06/15/science.abc |
| | | | | | | | | | | 7424) |
| mAb-100 | EIVMTQSPATLSVSPGETATLSCRASQ | IGHV3-30 | IGHJ4 | IGKV3-11 | IGKJ1 | 2882 | ARDNALQNA | 4072 | QRRGDGY | Anna Wec et al., 2020 |
| | SVGRFMGWYQQKPGQAPRLLIFDAS | (Human) | (Human) | (Human) | (Human) | | QIGYLDY | | N | (https://science.science |
| | NRVTGVPDRFRGSGSGTDFILTINSLEP | | | | | | | | | mag.org/content/early/ |
| | EDSASYYCQRRGDGYNFGQGTKVEIK | | | | | | | | | 2020/06/15/science.abc |
| | | | | | | | | | | 7424) |
| mAb-101 | ETTLTQSPLSLPVTLGQPASISCRSSQG | IGHV1-69 | IGHJ6 | IGKV2-30 | IGKJ1 | 2883 | VLDSDPYTAT | 4073 | MQGTEW | Anna Wec et al., 2020 |
| | LVHSDGNTYLNWFQQRPGQSPRRLIY | (Human) | (Human) | (Human) | (Human) | | FSHNHYWYA | | PRT | (https://science.science |
| | KVSNRDSGVPDRFSGSGSGTDFTLKIS | | | | | | MDV | | | mag.org/content/early/ |
| | RVEAEDIGVYYCMQGTEWPRTFGQG | | | | | | | | | 2020/06/15/science.abc |
| | TKVEIK | | | | | | | | | 7424) |
| mAb-102 | DIRVTQSPLSLPVTLGQPASISCRSSQRI | IGHV1-69 | IGHJ6 | IGKV2-30 | IGKJ3 | 2884 | ARDPSIHYTG | 4074 | MQGTEW | Anna Wec et al., 2020 |
| | VHTDGNTYLNWFLQRPGQSPRRLIYK | (Human) | (Human) | (Human) | (Human) | | NHHWYDLDI | | PRT | (https://science.science |
| | VSNRDSGVPDRFSGSGSGTDFTLKISR | | | | | | | | | mag.org/content/early/ |
| | VEAEDVGIYYCMQGTEWPRTFGQGT | | | | | | | | | 2020/06/15/science.abc |
| | KVDIK | | | | | | | | | 7424) |
| mAb-103 | EIVMTQSPSSLSASVGDRVTITCRAGH | IGHV7-4- | IGHJ4 | IGKV1-39 | IGKJ3 | 2885 | AREEHYDFSS | 4075 | QQSYSTPY | Anna Wec et al., 2020 |
| | TISTYLNWYQQKPGKAPKILISGASSLQ | 1 | (Human) | (Human) | (Human) | | GYFRPAY | | T | (https://science.science |
| | SGVPSRFSGSGSGTDFTLTIGSLQPEDF | (Human) | | | | | | | | mag.org/content/early/ |
| | ATYYCQQSYSTPYTFGQGTKVDIK | | | | | | | | | 2020/06/15/science.abc |
| | | | | | | | | | | 7424) |
| mAb-104 | QPVLTQPASVSGSPGQSSTLSCSGTSS | IGHV4-34 | IGHJ4 | IGLV2-23 | IGLJ3 | 2886 | ARGMTSPVV | 4076 | CSYAGSGT | Anna Wec et al., 2020 |
| | DVGSYDLVSWYQQHPGKAPKLMIYE | (Human) | (Human) | (Human) | (Human) | | HLYSSGRPSR | | WI | (https://science.science |
| | GTKRPSGVSDRFSGSTSGNTASLTISGL | | | | | | WFDF | | | mag.org/content/early/ |
| | QAEDEANYYCCSYAGSGTWIFGGGTK | | | | | | | | | 2020/06/15/science.abc |
| | LTVL | | | | | | | | | 7424) |
| mAb-105 | EIVLTQSPGTLSLSPGDRVTLFCRASQN | IGHV4-4 | IGHJ6 | IGKV3-20 | IGKJ1 | 2887 | ARSFISFDSS | 4077 | HQYGSSP | Anna Wec et al., 2020 |
| | IANNHLAWYQQKPGQAPRVLIYGAST | (Human) | (Human) | (Human) | (Human) | | GHPYYYYAMD | | WT | (https://science.science |
| | TATDIPDRFSGRVSGTDFTLTISRLDPE | | | | | | V | | | mag.org/content/early/ |
| | DFAVYYCHQYGSSPWTFGQGTKLEIK | | | | | | | | | 2020/06/15/science.abc |
| | | | | | | | | | | 7424) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| mAb-106 | Ab | SARS-CoV2 | 229E, | | | S; Unk | B-cells; SARS-CoV1 | 782 | QVQLVESGGGSVQAGGSLRLSCAASGSI | 1849 |
| | | (weak) | HKU1, | | | | Human Patient | | SSITYLGWFRQAPGKEREGVAALITDSG | |
| | | | NL63, | | | | | | RTYYADSVKGRFTVSLDNAKNTVYLQM | |
| | | | OC43, | | | | | | NSLKPEDTALYYCAAAEWGYEWPLYYAS | |
| | | | SARS- | | | | | | SWYWGQGTQVTVSS | |
| | | | CoV1 | | | | | | | |
| mAb-107 | Ab | SARS-CoV1, | 229E, | | | S; Unk | B-cells; SARS-CoV1 | 783 | QVQLVESGGGSVQAGGSLRLSCAASGSI | 1850 |
| | | SARS-CoV2 | HKU1, | | | | Human Patient | | SSITYLGWFRQAPGKEREGVAALITDSG | |
| | | (weak | NL63, | | | | | | RTYYADSVKGRFTVSLDNAKNTVYLQM | |
| | | | OC43 | | | | | | NSLKPEDTALYYCAAAEWGYEWPLYYAS | |
| | | | | | | | | | SWYWGQGTQVTVSS | |
| mAb-108 | Ab | SARS-CoV2 | 229E, | | | S; Unk | B-cells; SARS-CoV1 | 784 | QVQLVESGGGSVQAGGSLRLSCAASGSI | 1851 |
| | | (weak) | HKU1, | | | | Human Patient | | SSITYLGWFRQAPGKEREGVAALITDSG | |
| | | | NL63, | | | | | | RTYYADSVKGRFTVSLDNAKNTVYLQM | |
| | | | OC43, | | | | | | NSLKPEDTALYYCAAAEWGYEWPLYYAS | |
| | | | SARS- | | | | | | SWYWGQGTQVTVSS | |
| | | | CoV1 | | | | | | | |
| mAb-109 | Ab | SARS-CoV1, | 229E, | | | S; Unk | B-cells; SARS-CoV1 | 785 | QVQLVESGGGSVQAGGSLRLSCAASGSI | 1852 |
| | | SARS-CoV2 | HKU1, | | | | Human Patient | | SSITYLGWFRQAPGKEREGVAALITDSG | |
| | | (weak) | NL63, | | | | | | RTYYADSVKGRFTVSLDNAKNTVYLQM | |
| | | | OC43 | | | | | | NSLKPEDTALYYCAAAEWGYEWPLYYAS | |
| | | | | | | | | | SWYWGQGTQVTVSS | |
| mAb-11 | Ab | SARS-CoV1, | 229E, | | | S; Unk | B-cells; SARS-CoV1 | 786 | QVQLVESGGGSVQAGGSLRLSCAASGSI | 1853 |
| | | SARS-CoV2 | HKU1, | | | | Human Patient | | SSITYLGWFRQAPGKEREGVAALITDSG | |
| | | | NL63, | | | | | | RTYYADSVKGRFTVSLDNAKNTVYLQM | |
| | | | OC43 | | | | | | NSLKPEDTALYYCAAAEWGYEWPLYYAS | |
| | | | | | | | | | SWYWGQGTQVTVSS | |
| mAb-110 | Ab | SARS-CoV1, | 229E, | | | S; Unk | B-cells; SARS-CoV1 | 787 | EVQLVESGGGVVQPGRSLRLSCAASGFT | 1854 |
| | | SARS-CoV2 | HKU1, | | | | Human Patient | | FSSYGMHWVRQAPGKGLEWVAVIWY | |
| | | (weak) | NL63, | | | | | | DGSNKYYADSVKGRFTISRDNSKNTLYL | |
| | | | OC43 | | | | | | QMNSLRAEDTAVYYCARVGSGRVYWG | |
| | | | | | | | | | QGTLVTVSS | |
| mAb-111 | Ab | 229E | HKU1, | | | S; Unk | B-cells; SARS-CoV1 | 788 | QVQLVESGPGLVRPSGTLSVTCAVSGDS | 1855 |
| | | (weak), | NL63, | | | | Human Patient | | ISSDYWCTWVRQSPGKGLEWIGKISHS | |
| | | SARS-CoV1, | OC43 | | | | | | GSLNYNPSLKSRVTMSVDKSKNHFSLKL | |
| | | SARS-CoV2 | | | | | | | ASVTAADTAVYYCARVRIGASHHNFWS | |
| | | (weak) | | | | | | | GYYTDAFDIWGQGTTVTVSS | |
| mAb-112 | Ab | 229E | OC43 | | | S; Unk | B-cells; SARS-Cov1 | 789 | QVQLVQSGAEVKKPGESLKISCKASGYS | 1856 |
| | | (weak), | | | | | Human Patient | | FTNYWVGWVRQMPAKGLEWMGIIWP | |
| | | HKU1 | | | | | | | DDSDTRYRPSFQGQVTISVDKSISTAYLH | |
| | | (weak), | | | | | | | WNSLKASDNGMYFCARAPLASCSGGRC | |
| | | NL63, SARS- | | | | | | | PTYNRFDLWGQGTLVTVSS | |
| | | CoV1, SARS- | | | | | | | | |
| | | CoV2 | | | | | | | | |
| | | (weak) | | | | | | | | |
| mAb-113 | Ab | 229E, HKU1 | OC43 | | | S; Unk | B-cells; SARS-CoV1 | 790 | QVQLVQSGAEVKKPGSSMKVSCKASGV | 1857 |
| | | (weak) | | | | | Human Patient | | NFRSYSFSWVRQAPGQGLEWMGGVIP | |
| | | NL63, SARS- | | | | | | | YFPTANYADKFRDRVTITADESTGTVYLD | |
| | | CoV1, SARS- | | | | | | | MSSLRSEDTAVYFCASEYFDGRSYHSFC | |
| | | CoV2 | | | | | | | GLDVWGQGTTVTVSS | |
| | | (weak) | | | | | | | | |
| mAb-114 | Ab | 229E, HKU1, | OC43 | | | S; Unk | B-cells; SARS-CoV1 | 791 | QVQLVESGGGLVQPGGSLRLSCAASGF | 1858 |
| | | NL63, SARS- | | | | | Human Patient | | TFSNYAMTWVRQAPGKGLKWVSGINP | |
| | | CoV1, SARS- | | | | | | | SGDATFYTDSVKGQFTISRDNSKNILYLQ | |
| | | CoV2 | | | | | | | MNRLRADDTAIYYCAKGLSFYGSGSDAF | |
| | | (weak) | | | | | | | DVWGQGTTVTVSS | |
| mAb-115 | Ab | 229E, HKU1 | OC43 | | | S; Unk | B-cells; SARS-CoV1 | 792 | QVQLVQSGAEVKKPGASVKVSCKASGY | 1859 |
| | | (weak), | | | | | Human Patient | | TFTDYYVHWVRQVPGQGLEWMGWIS | |
| | | NL63, SARS- | | | | | | | PDSGDTIRAQNFQGRVTMTRDTSMINT | |
| | | CoV1, SARS- | | | | | | | AYMEVNRLRTDDTAIYYCARDLISVIRGL | |
| | | CoV2 | | | | | | | GGGMDVWGQGTTVTVSS | |
| | | (weak) | | | | | | | | |
| mAb-116 | Ab | 229E | OC43 | | | S; Unk | B-cells; SARS-CoV1 | 793 | QVQLVQSGAEVKKPGASVKVSCKTSGY | 1860 |
| | | (weak), | | | | | Human Patient | | TFTGHYIHWVRQAPGQGLEWMGWIN | |
| | | HKU1 | | | | | | | PASGDTNYAQKFQGRVTKTRDTSITTAY | |
| | | (weak), | | | | | | | MELNRLRSDDTAVYYCARGGPLPWSDL | |
| | | NL63 | | | | | | | DIVGTFDYWGQGTLVTVSS | |
| | | (weak), | | | | | | | | |
| | | SARS-CoV1, | | | | | | | | |
| | | SARS-CoV2 | | | | | | | | |
| | | (weak) | | | | | | | | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| mAb-106 | ETTLTQSPATLSVSPGETATLSCRASQS | IGHV3-30 | IGHJ4 | IGKV3-11 | IGKJ1 | 2888 | ARDNALQNA | 4078 | QRRGDGY | Anna Wec et al., 2020 |
| | VGRFMGWYQQKPGQAPRLLIFDASN | (Human) | (Human) | (Human) | (Human) | | QIGYLDY | | N | (https://science.science |
| | RVTGVPDRFRGSGSGTDFILTINSLEPE | | | | | | | | | mag.org/content/early/ |
| | DSASYYCQRRGDGYNFGQGTKVEIK | | | | | | | | | 2020/06/15/science.abc |
| | | | | | | | | | | 7424) |
| mAb-107 | DIRMTQSPLSLPVDLGQSASISCRSSQ | IGHV1-69 | IGHJ6 | IGKV2-30 | IGKJ2 | 2889 | ARDPSILNTG | 4079 | MQATDW | Anna Wec et al., 2020 |
| | RVVHTNGNTYLHWFHQRPGQAPRRL | (Human) | (Human) | (Human) | (Human) | | NHHWYDLD | | PRT | (https://science.science |
| | IYKVSNRESGVPDRFSGSGSGTDFTLRI | | | | | | M | | | mag.org/content/early/ |
| | SRVEAEDVGVYYCMQATDWPRTFGQ | | | | | | | | | 2020/06/15/science.abc |
| | GTKLEIK | | | | | | | | | 7424) |
| mAb-108 | DIVMTQSPLSLSVTLGQAASISCTCSQT | IGHV1-69 | IGHJ6 | IGKV2-30 | IGKJ1 | 2890 | ARDPTFLNSG | 4080 | MQTTDWP | Anna Wec et al., 2020 |
| | AVHSDGNTYLNWFHQRPGQSPRRLIY | (Human) | (Human) | (Human) | (Human) | | NHFWYDVDI | | RT | (https://science.science |
| | KVSNRDSGVPDRFSGSGSGTDFTLKIN | | | | | | | | | mag.org/content/early/ |
| | RVEAEDVGIYYCMQTTDWPRTFGQG | | | | | | | | | 2020/06/15/science.abc |
| | TKVEIK | | | | | | | | | 7424) |
| mAb-109 | ETTLTQSPVTLPVTLGQPASISCTSSRW | IGHV1-69 | IGHJ4 | IGKV2-30 | IGKJ2 | 2891 | ARDASFPNTG | 4081 | MQGTEW | Anna Wec et al., 2020 |
| | LVHTNGNTYLNWFHQRPGQSPRRLIY | (Human) | (Human) | (Human) | (Human) | | NHFWYDFDL | | PRT | (https://science.science |
| | QVSNRDSGVPDRFSGSGSGTDFTLQIS | | | | | | | | | mag.org/content/early/ |
| | RVEAEDVGVYYCMQGTEWPRTFGQ | | | | | | | | | 2020/06/15/science.abc |
| | GTKLEIK | | | | | | | | | 7424) |
| mAb-11 | EIVLTQSPSSLSASVGDRVTITCQASQD | IGHV3-30 | IGHJ4 | IGKV1-33 | IGKJ5 | 2892 | AKKGSPYCGV | 4082 | QQFEDLP | Anna Wec et al., 2020 |
| | IRKCLNWYQHIPGKAPKLLIHDASSLES | (Human) | (Human) | (Human) | (Human) | | DCYKGYFDY | | IT | (https://science.science |
| | GVPSRFSGSGSGTDFSFTINSLHPEDIA | | | | | | | | | mag.org/content/early/ |
| | TYYCQQFEDLPITFGQGTRLEIK | | | | | | | | | 2020/06/15/science.abc |
| | | | | | | | | | | 7424) |
| mAb-110 | DIVMTQSPDSLAVSLGERATINCKSSQ | IGHV3-33 | IGHJ4 | IGKV4-1 | IGKJ1 | 2893 | ARVGSGRVY | 4083 | QQYYSTP | Anna Wec et al., 2020 |
| | SVLYSSNNKNYLAWYQQKPGQPPKLLI | (Human) | (Human) | (Human) | (Human) | | | | YT | (https://science.science |
| | YWASTRESGVPDRFSGSGSGTDFTLTI | | | | | | | | | mag.org/content/early/ |
| | SSLQAEDVAVYYCQQYYSTPYTFGQGT | | | | | | | | | 2020/06/15/science.abc |
| | KVEIK | | | | | | | | | 7424) |
| mAb-111 | DIVVTQSPGTLSLSPGERAALSCRASQS | (Human) | IGHJ3 | IGKV3-20 | IGKJ1 | 2894 | ARVRIGASHH | 4084 | QQYGTSP | Anna Wec et al., 2020 |
| | VGNNYLAWYQQKPGQAPRLLIYGATS | IGHV4-4 | (Human) | (Human) | (Human) | | NFWSGYYTD | | VYT | (https://science.science |
| | RATGIPDRFSGSGSGTDFTLTISRLEPE | | | | | | AFDI | | | mag.org/content/early/ |
| | DFAVYYCQQYGTSPVYTFGQGTKVEIK | | | | | | | | | 2020/06/15/science.abc |
| | | | | | | | | | | 7424) |
| mAb-112 | QPVLTQPPSVSAAPGQKVTISCSGSSS | IGHV5-51 | IGHJ4 | IGLV1-51 | IGLJ3 | 2895 | ARAPLASCSG | 4085 | GTWDFSL | Anna Wec et al., 2020 |
| | NIENNYVSWYQQLPGAAPKLLIYDNN | (Human) | (Human) | (Human) | (Human) | | GRCPTYNRFD | | SAGV | (https://science.science |
| | KRPSGIPDRFSGSKSGTSATLGITGLQT | | | | | | L | | | mag.org/content/early/ |
| | GDEADYYCGTWDFSLSAGVFGGGTKL | | | | | | | | | 2020/06/15/science.abc |
| | TVL | | | | | | | | | 7424) |
| mAb-113 | ETTLTQSPLSLPVTLGQPASISCRSSQG | IGHV1-69 | IGHJ6 | IGKV2-30 | IGKJ1 | 2896 | ASEYFDGRSY | 4086 | MQGTEW | Anna Wec et al., 2020 |
| | LVHTNGNLYLNWFHQRPGQSPRRLIY | (Human) | (Human) | (Human) | (Human) | | HSFCGLDV | | PRT | (https://science.science |
| | QVSNRDSGVPDRFSGSGSGTDFTLRIS | | | | | | | | | mag.org/content/early/ |
| | RVEAEDVGIYYCMQGTEWPRTFGQG | | | | | | | | | 2020/06/15/science.abc |
| | TKVEIK | | | | | | | | | 7424) |
| mAb-114 | DIQLTQSPSTLSASVGDSVTITCRASQSI | IGHV3-23 | IGHJ6 | IGKV1-5 | IGKJ4 | 2897 | AKGLSFYGSG | 4087 | QQYKSPL | Anna Wec et al., 2020 |
| | SSWLAWYQQKPGKAPKLLIYKASSLET | (Human) | (Human) | (Human) | (Human) | | SDAFDV | | S | (https://science.science |
| | GVPSRFSGSGSGTEFTLTISSLQPDDFA | | | | | | | | | mag.org/content/early/ |
| | TYYCQQYKSPLSFGGGTKVEIK | | | | | | | | | 2020/06/15/science.abc |
| | | | | | | | | | | 7424) |
| mAb-115 | DIQVTQSPATLSLSPGERATLSCRASQS | IGHV1-2 | IGHJ6 | IGKV3-11 | IGKJ5 | 2898 | ARDLISVIR | 4088 | QQRSDWH | Anna Wec et al., 2020 |
| | VSSYLAWYQQKPGQAPRLLIYDASKR | (Human) | (Human) | (Human) | (Human) | | GLGGGMDV | | PIT | (https://science.science |
| | ATGIPARFSGSGSGTDFTLTISSLEPEDF | | | | | | | | | mag.org/content/early/ |
| | AVYYCQQRSDWHPITFGQGTRLEIK | | | | | | | | | 2020/06/15/science.abc |
| | | | | | | | | | | 7424) |
| mAb-116 | DIQVTQSPSSLSASVGDRVTITCRASQS | IGHV1-2 | IGHJ4 | IGKV1-39 | IGKJ3 | 2899 | ARGGPLPWS | 4089 | QQSYSTP | Anna Wec et al., 2020 |
| | ISSSLNWYQQKPGKAPTLLIYTASNLQS | (Human) | (Human) | (Human) | (Human) | | DLDIVGTFDY | | G | (https://science.science |
| | GVPSRFSGSGSGTDFTLTITSLQPEDFA | | | | | | | | | mag.org/content/early/ |
| | TYYCQQSYSTPGFGPGTKVDIK | | | | | | | | | 2020/06/15/science.abc |
| | | | | | | | | | | 7424) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| mAb-117 | Ab | 229E, HKU1 | OC43 | | | S; Unk | B-cells; SARS-CoV1 | 794 | EVQLVESGGGVVQPGKSLRLSCVPSGFS | 1861 |
| | | NL63, SARS- | | | | | Human Patient | | FGTYGMHWVRQAPGKGPEWLAVMW | |
| | | CoV1, SARS- | | | | | | | YDGITQYYADSVKGRFTISRDNSKETLYL | |
| | | CoV2 | | | | | | | QMNSLTADDTGIYYCVKDQSSGDRLLYL | |
| | | (weak) | | | | | | | GYFDLWGPGALVTVSS | |
| mAb-118 | Ab | 229E, HKU1 | OC43 | | | S; Unk | B-cells; SARS-CoV1 | 795 | QVQLVQSGGGVVQPGKSLRLSCVASGF | 1862 |
| | | (weak), | | | | | Human Patient | | SFGTYGMHWVRQAPGKGPEWLAVM | |
| | | NL63 | | | | | | | WYDGITQYYADSVKGRFTISRDNSKETLY | |
| | | (weak), | | | | | | | LQMNSLTADDTGVYYCVKDQSSGDRLL | |
| | | SARS-CoV1, | | | | | | | YLGYFDLWGPGTLVTVSS | |
| | | SARS-CoV2 | | | | | | | | |
| | | (weak) | | | | | | | | |
| mAb-119 | Ab | 229E, HKU1 | OC43 | | | S; Unk | B-cells; SARS-CoV1 | 796 | QVQLQESGPGLVKPSGTLSLTCAVSGAS | 1863 |
| | | (weak), | | | | | Human Patient | | VSSDHWWSWVRQSPGKGLEWIGEVY | |
| | | NL63 | | | | | | | HSGSTNYNPSLKSRVTISLDQSNNQFSLK | |
| | | (weak), | | | | | | | LTSVTAADTAIYYCATMWGGLCTASNCY | |
| | | SARS-CoV1, | | | | | | | GNPMDVWGQGTTVTVSS | |
| | | SARS-CoV2 | | | | | | | | |
| | | (weak) | | | | | | | | |
| mAb-12 | Ab | SARS-CoV1, | 229E, | | | S; Unk | B-cells; SARS-CoV1 | 797 | QVQLVQSGAEVRKPGSSVKLSCKASGG | 1864 |
| | | SARS-CoV2 | HKU1, | | | | Human Patient | | TFSTHAISWVRQAPGQRPEWMGGIMP | |
| | | | NL63, | | | | | | IFGESKDTQKFQGRVTFTADESTTTAYM | |
| | | | OC43 | | | | | | ELRSLRSDDTAIYYCVRDSDPYTATVTSN | |
| | | | | | | | | | HYWYAMDVWGQGTTVTVSS | |
| mAb-120 | Ab | 229E | OC43 | | | S; Unk | B-cells; SARS-CoV1 | 798 | QVQLQQWGAGVLKPSETLSLTCAVYGG | 1865 |
| | | (weak), | | | | | Human Patient | | SFRGFFWSWIRQPPGKGLEWIGQITHS | |
| | | HKU1 | | | | | | | GSTNYNSSLKSRLTISVDTSKNQFSLNLSS | |
| | | (weak), | | | | | | | VTAADTAIYYCARGQGGYDLRRVGYGLT | |
| | | NL63 | | | | | | | SWFDPWGQGILVTVSS | |
| | | (weak), | | | | | | | | |
| | | SARS-CoV1, | | | | | | | | |
| | | SARS-CoV2 | | | | | | | | |
| | | (weak) | | | | | | | | |
| mAb-121 | Ab | 229E, HKU1 | OC43 | | | S; Unk | B-cells; SARS-CoV1 | 799 | QVQLVQSGAEVKKPGASVKVSCKASGY | 1866 |
| | | (weak), | | | | | Human Patient | | TFTSYYIHWVRQAPGQGLEWLGVIHPS | |
| | | NL63 | | | | | | | GGSTTFAQKFQGRVTMTRDTSTSTVYM | |
| | | (weak), | | | | | | | ELGSLRSDDTAVYYCARVLAGSSHEWQL | |
| | | SARS-CoV1, | | | | | | | THDAFDIWGQGTTVTVSS | |
| | | SARS-CoV2 | | | | | | | | |
| | | (weak) | | | | | | | | |
| mAb-122 | Ab | 229E | OC43 | | | S; Unk | B-cells; SARS-CoV1 | 800 | EVQLVESGAEVKKPGASVILSCKASGYTV | 1867 |
| | | (weak), | | | | | Human Patient | | TNYYIHWVRQAPGQGLEWMGWIDPD | |
| | | HKU1 | | | | | | | SGVTNFAEKFQGRVTMTRDTSISTAYME | |
| | | (weak), | | | | | | | LRWLESDDTAVYYCAKDLITVIRGLGGG | |
| | | NL63 | | | | | | | MDVWGQGTTVTVSS | |
| | | (weak), | | | | | | | | |
| | | SARS-CoV1, | | | | | | | | |
| | | SARS-CoV2 | | | | | | | | |
| | | (weak) | | | | | | | | |
| mAb-123 | Ab | 229E | NL63, | | | S; Unk | B-cells; SARS-CoV1 | 801 | EVQLLESGAEVKKPGSSVKVSCKASGGT | 1868 |
| | | (weak), | OC43 | | | | Human Patient | | FSSDAISWVRQAPGQGLEWMGGIIPIF | |
| | | HKU1 | | | | | | | GTTNYAQKFQGRVTITADESTNTAYMEL | |
| | | (weak), | | | | | | | SSLRSEDTAVYYCARDGPYDSGGYHLNH | |
| | | SARS-CoV1, | | | | | | | WGQGTLVTVSS | |
| | | SARS-CoV2 | | | | | | | | |
| | | (weak) | | | | | | | | |
| mAb-124 | Ab | HKU1 | 229E, | | | S; Unk | B-cells; SARS-CoV1 | 802 | EVQLLESGGGVVNPGGSMRLSCAGSGF | 1869 |
| | | (weak), | NL63, | | | | Human Patient | | TFSDHYMGWIRQAPGKGLEVISYISSSG | |
| | | SARS-CoV1, | OC43 | | | | | | SFIRDADSVKGRFTISRDNAKNSVYLQM | |
| | | SARS-CoV2 | | | | | | | NSLRAEDTAVYYCARMGPYGSGTFDYW | |
| | | (weak) | | | | | | | GQGTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| mAb-117 | EIVMTQSPATLSLSPGERATLSCRASQS | IGHV3-33 | IGHJ2 | IGKV3-11 | IGKJ3 | 2900 | VKDQSSGDRL | 4090 | QQRAKWP | Anna Wec et al., 2020 |
| | VSIYLAWYQQKPGQAPRLLIYDASNRA | (Human) | (Human) | (Human) | (Human) | | LYLGYFDL | | PRVT | (https://science.science |
| | TGVPARFSGSGSGTDFTLTINNLEPED | | | | | | | | | mag.org/content/early/ |
| | FAIYYCQQRAKWPPRVTFGPGTKVDIK | | | | | | | | | 2020/06/15/science.abc |
| | | | | | | | | | | 7424) |
| mAb-118 | DIVLTQSPATLSLSPGERATLSCRASQS | IGHV3-33 | IGHJ2 | IGKV3-11 | IGKJ3 | 2901 | VKDQSSGDRL | 4091 | QQRAKWP | Anna Wec et al., 2020 |
| | VSIYLAWYQQKPGQAPRLLIYDASNRA | (Human) | (Human) | (Human) | (Human) | | LYLGYFDL | | PRVI | (https://science.science |
| | TGIPARFSGSGSGTDFTLTINNLEPEDF | | | | | | | | | mag.org/content/early/ |
| | AIYYCQQRAKWPPRVIFGPGTKVEIK | | | | | | | | | 2020/06/15/science.abc |
| | | | | | | | | | | 7424) |
| mAb-119 | SYELTQPPSVSVSPGQTARITCSGDALP | IGHV4-4 | IGHJ6 | IGLV3-10 | IGLJ3 | 2902 | ATMWGGLCT | 4092 | YSTDSTAN | Anna Wec et al., 2020 |
| | RRYAYWYQQRSGQAPVLVIYEDNKRP | (Human) | (Human) | (Human) | (Human) | | ASNCYGNPM | | YKV | (https://science.science |
| | SGIPERFSAFSSGTMATLTISGAQVEDE | | | | | | DV | | | mag.org/content/early/ |
| | ADYYCYSTDSTANYKVFGGGTKLTVL | | | | | | | | | 2020/06/15/science.abc |
| | | | | | | | | | | 7424 |
| mAb-12 | ETTLTQSPLSLPVTLGQPASISCRSSQG | IGHV1-69 | IGHJ6 | IGKV2-30 | IGKJ3 | 2903 | VRDSDPYTAT | 4093 | MQGTEW | Anna Wec et al., 2020 |
| | LVHSNGNTYVNWFHQRPGQSPRRLIY | (Human) | (Human) | (Human) | (Human) | | VTSNHYWYA | | PRT | (https://science.science |
| | EVSNRDSGVPDRFSGSGSGTDFTLKIS | | | | | | MDV | | | mag.org/content/early/ |
| | RVEAEDIGVYYCMQGTEWPRTFGQG | | | | | | | | | 2020/06/15/science.abc |
| | TKVDIK | | | | | | | | | 7424) |
| mAb-120 | QSVLIQPASVSGSPGQSITISCTGSSSD | IGHV4-34 | IGHJ5 | IGLV2-23 | IGLJ3 | 2904 | ARGQGGYDL | 4094 | CSYAGSSA | Anna Wec et al., 2020 |
| | VGSYNLVSWYQQHPGKAPKLMIYEGY | (Human) | (Human) | (Human) | (Human) | | RRVGYGLTS | | VVVV | (https://science.science |
| | KRPSGVSNRFSGSKSGNTASLTISGLQ | | | | | | WFDP | | | mag.org/content/early/ |
| | AEDEADYYCCSYAGSSAVVVVFGGGT | | | | | | | | | 2020/06/15/science.abc |
| | KLTVL | | | | | | | | | 7424) |
| mAb-121 | DIRVTQSPDSLAVSLGERATINCRTSQS | IGHV1-46 | IGHJ3 | IGKV4-1 | IGKJ3 | 2905 | ARVLAGSSHE | 4095 | QQYYSTPY | Anna Wec et al., 2020 |
| | VLYSSNNKNYLGWYQQKPGQPPKLLI | (Human) | (Human) | (Human) | (Human) | | WQLTHDAFD | | T | (https://science.science |
| | YWASTRESGVPDRFSGSGSGTDFTLTI | | | | | | | | | mag.org/content/early/ |
| | SSLQAEDVAVYYCQQYYSTPYTFGQGT | | | | | | | | | 2020/06/15/science.abc |
| | KVDIK | | | | | | | | | 7424) |
| mAb-122 | EIVLTQSPATLSLSPGERATLSCRASQS | IGHV1-2 | IGHJ6 | IGKV3-11 | IGKJ5 | 2906 | AKDLITVIRGL | 4096 | LQRSDWH | Anna Wec et al., 2020 |
| | VSTYLAWYQQKPGQAPRLLIYDASNR | (Human) | (Human) | (Human) | (Human) | | GGGMDV | | PIT | (https://science.science |
| | ATGIPARFSGSGSGTDFTLTISSLEPEDF | | | | | | | | | mag.org/content/early/ |
| | ALYYCLQRSDWHPITFGQGTRLEIK | | | | | | | | | 2020/06/15/science.abc |
| | | | | | | | | | | 7424) |
| mAb-123 | DIQLTQSPATLSLSPGERATLSCRANQS | IGHV1-69 | IGHJ1 | IGKV3-11 | IGKJ4 | 2907 | ARDGPYDSG | 4097 | QQRSNWP | Anna Wec et al., 2020 |
| | VSNFLAWYQQKPGQAPRHLIYDASNR | (Human) | (Human) | (Human) | (Human) | | GYHLNH | | PRLT | (https://science.science |
| | ATGIPARFSGSGSGTDFTLTISSLEPEDF | | | | | | | | | mag.org/content/early/ |
| | AVYYCQQRSNWPPRLTFGGGTKVEIK | | | | | | | | | 2020/06/15/science.abc |
| | | | | | | | | | | 7424) |
| mAb-124 | DIVMTQTPGTLSVSPGERATLSCRASQ | IGHV3-11 | IGHJ4 | IGKV3-20 | IGKJ3 | 2908 | ARMGPYGSG | 4098 | LQYSLATT | Anna Wec et al., 2020 |
| | IINRSQLGWYQHKPGQPPRLLIFDSSK | (Human) | (Human) | (Human) | (Human) | | TFDY | | | (https://science.science |
| | RATGTPDRFSASGSETDFTLTISGVEPE | | | | | | | | | mag.org/content/early/ |
| | DSGVYYCLQYSLATTFGPGTKVEIK | | | | | | | | | 2020/06/15/science.abc |
| | | | | | | | | | | 7424) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| mAb-125 | Ab | (weak) | OC43 | | | S; Unk | B-cells; SARS-CoV1 | 803 | EVQLVESGAEVRKPGSSVKVSCKATGGT | 1870 |
| | | 229E, HKU1 | | | | | Human Patient | | FSSYGITWVRQAPGQGLEWMGRIIPTL | |
| | | (weak), | | | | | | | GRTNYAQKFQGRVTITADKSTSTAHMEL | |
| | | NL63 | | | | | | | SSLRSEDTAVYYCARDLSTLQPDAIVNFD | |
| | | (weak), | | | | | | | YWGQGTLVTVSS | |
| | | SARS-CoV1, | | | | | | | | |
| | | SARS-CoV2 | | | | | | | | |
| mAb-126 | Ab | HKU1 | 229E, | | | S; Unk | B-cells; SARS-CoV1 | 804 | QVQLVQSGAEVKKPGSSVKVSCKTSGG | 1871 |
| | | (weak), | NL63, | | | | Human Patient | | SFTSYVLSWVRQAPGQGLEWMGRIVP | |
| | | SARS-CoV1, | OC43 | | | | | | NLGVANYAQKFQDRVTITADKSTTTAYL | |
| | | SARS-CoV2 | | | | | | | ELRSLRSEDTAVYYCARDLYYDNGGYNY | |
| | | (weak) | | | | | | | LDYWGPGTLVTVSS | |
| mAb-127 | Ab | 229E | NL63, | | | S; Unk | B-cells; SARS-CoV1 | 805 | QVQLVESGGGLVQPGGSLRLSCAASGF | 1872 |
| | | (weak), | OC43 | | | | Human Patient | | TFSNYNMVWVRQAPGKGLEWISYISSSS | |
| | | HKU1 | | | | | | | STIYYADSVKGRFIISRDNAKNSLHLQMN | |
| | | (weak), | | | | | | | SLRDEDTAVYYCVRDYCNSVSCYTYYYIG | |
| | | SARS-CoV1, | | | | | | | MDVWGQGTTVTVSS | |
| | | SARS-CoV2 | | | | | | | | |
| | | (weak) | | | | | | | | |
| mAb-128 | Ab | 229E | OC43 | | | S; Unk | B-cells; SARS-CoV1 | 806 | EVQLVQSGAEVKKPGASVKVSCKASGYS | 1873 |
| | | (weak), | | | | | Human Patient | | STNYGFSWVRQAPGQGLEWMGWISV | |
| | | HKU1, NL63 | | | | | | | HSGNANFAQKFQGRITMTTDTSTTTAY | |
| | | (weak), | | | | | | | MELRNLRSDDTATYYCATSASSYSRYYFG | |
| | | SARS-CoV1, | | | | | | | LDVWGQGTTVTVSS | |
| | | SARS-CoV2 | | | | | | | | |
| | | (weak) | | | | | | | | |
| mAb-129 | Ab | HKU1, SARS- | 229E, | | | S; Unk | B-cells; SARS-CoV1 | 807 | EVQLLESGPGLVKPSQTLSLTCTVSNGFI | 1874 |
| | | CoV1 | NL63, | | | | Human Patient | | SSGEYYWSWIRQSPGKGLEWIGYISHSG | |
| | | (weak), | OC43 | | | | | | STYYNRSLKSRVTISLDTSRNQFSLNLSSV | |
| | | SARS-CoV2 | | | | | | | TAADTAVYYCARDLAKWSYGYYYSGMD | |
| | | (weak) | | | | | | | VWGQGTTVTVSS | |
| mAb-13 | Ab | SARS-CoV1, | 229E, | | | S; Unk | B-cells; SARS-CoV1 | 808 | QVQLVQSGAEVKKPGSSVKVSCKISGGT | 1875 |
| | | SARS-CoV2 | HKU1, | | | | Human Patient | | FKNSAFSWARQVPGQGFQWMGGIIP | |
| | | | NL63, | | | | | | MFGVPHSVQMFQGRVTLTADESTSAVY | |
| | | | OC43 | | | | | | MELSGLTSDDTAVYYCAREEYSGTVHNF | |
| | | | | | | | | | FGMDVWGQGTTVTVSS | |
| mAb-130 | Ab | HKU1 | 229E, | | | S; Unk | B-cells; SARS-CoV1 | 809 | EVQLVESGGGLVQPGRSLRLSCAASGFT | 1876 |
| | | (weak), | OC43 | | | | Human Patient | | FDDYAMHWVRQVPGRGLEWVSGISW | |
| | | NL63 | | | | | | | NSGTINYADSVMGRFTISRDNAKNSLYL | |
| | | (weak), | | | | | | | QMNSLRAEDTALYYCAKDGRYCSGISCR | |
| | | SARS-CoV1 | | | | | | | TGMDVWGQGTTVTVSS | |
| | | (weak), | | | | | | | | |
| | | SARS-CoV2 | | | | | | | | |
| | | (weak) | | | | | | | | |
| mAb-131 | Ab | 229E | NL63, | | | S; Unk | B-cells; SARS-CoV1 | 810 | EVQLVESGGGLVQPGESLRLSCAASGEN | 1877 |
| | | (weak), | OC43 | | | | Human Patient | | FSPYGMNWVRQAPGKGLEWIAYIISGS | |
| | | HKU1 | | | | | | | GTIYYADSVKGRFTISRDNAQSSLYLQM | |
| | | (weak), | | | | | | | NSLRAEDTAVYYCARGLLDYLHDAFDIW | |
| | | SARS-CoV1 | | | | | | | GQGTMVTVSS | |
| | | (weak), | | | | | | | | |
| | | SARS-CoV2 | | | | | | | | |
| | | (weak) | | | | | | | | |
| mAb-132 | Ab | 229E, HKU1 | OC43 | | | S; Unk | B-cells; SARS-CoV1 | 811 | EVQLQESGPGLVKPSETLSLTCAVSGGS | 1878 |
| | | (weak), | | | | | Human Patient | | VSSDTDYWGWIRQPPGKGLEWIGSIHD | |
| | | NL63 | | | | | | | SERTYYDPSLKSRVTISVDTSKNQFSLRLS | |
| | | (weak), | | | | | | | SVTAADTALYFCASRHLDLLPIGSFDVW | |
| | | SARS-CoV1 | | | | | | | GRGTMVTVSS | |
| | | (weak), | | | | | | | | |
| | | SARS-CoV2 | | | | | | | | |
| | | (weak) | | | | | | | | |
| mAb-133 | Ab | HKU1 | 229E, | | | S; Unk | B-cells; SARS-CoV1 | 812 | QVQLLESGGGLVQPGRSLRLSCTASGFR | 1879 |
| | | (weak), | NL63, | | | | Human Patient | | FGDYTMTWFRQAPGKGLEWVGFIRSIA | |
| | | SARS-CoV1 | OC43 | | | | | | YGGTTEHAASVEGRFIISRDDSKSIAYLQ | |
| | | (weak), | | | | | | | MNSLKAEDTGVYFCTRGSGMFYGSSSG | |
| | | SARS-CoV2 | | | | | | | MDVWGQGTTVTVSS | |
| | | (weak) | | | | | | | | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| mAb-125 | QPVLTQSSSASASLGSSVKLTCTLSSGH | IGHV1-69 | IGHJ4 | IGLV4-60 | IGLJ6 | 2909 | ARDLSTLQPD | 4099 | ETWDSNL | Anna Wec et al., 2020 |
| | SSYILAWHQQQPGKAPRFLMKFEVG | (Human) | (Human) | (Human) | (Human) | | AIVNFDY | | KGV | (https://science.science |
| | GRYNKGSGVPDRFSGSSSGADRYLTIS | | | | | | | | | mag.org/content/early/ |
| | NLQSEDEADYYCETWDSNLKGVFGG | | | | | | | | | 2020/06/15/science.abc |
| | GTKVTVL | | | | | | | | | 7424) |
| mAb-126 | QPVLTQSSSASASLGSSVKLTCTLRSGH | IGHV1-69 | IGHJ4 | IGLV4-60 | IGLJ6 | 2910 | ARDLYYDNG | 4100 | EAWDNNN | Anna Wec et al., 2020 |
| | SSYIIAWHQQQPGKAPRFLMKVGHSG | (Human) | (Human) | (Human) | (Human) | | GYNYLDY | | LGV | (https://science.science |
| | SYNKGSGVPDRFSGSRSGADHYLTISN | | | | | | | | | mag.org/content/early/ |
| | LQPDDEADYYCEAWDNNNLGVFSGG | | | | | | | | | 2020/06/15/science.abc |
| | TKVTVL | | | | | | | | | 7424) |
| mAb-127 | DIQLTQSPSAMSASVGDRVTITCRASQ | IGHV3-48 | IGHJ6 | IGKV1-17 | IGKJ4 | 2911 | VRDYCNSVSC | 4101 | LQHNSYPL | Anna Wec et al., 2020 |
| | GINDNLAWFQQKPGKVPKRLIYAASN | (Human) | (Human) | (Human) | (Human) | | YTYYYIGMDV | | T | (https://science.science |
| | LQNGVPSRFSGSGSGTEFTLTISSLQPE | | | | | | | | | mag.org/content/early/ |
| | DFATYYCLQHNSYPLTFGGGTKLEIK | | | | | | | | | 2020/06/15/science.abc |
| | | | | | | | | | | 7424) |
| mAb-128 | DIRMTQSPLSLPVTPGEPASISCRSSQS | IGHV1-18 | IGHJ6 | IGKV2-28 | IGKJ3 | 2912 | ATSASSYSRYY | 4102 | MQALQTP | Anna Wec et al., 2020 |
| | LLHSNGYNYLDWYLQKPGQSPQLLIYL | (Human) | (Human) | (Human) | (Human) | | FGLDV | | GVT | (https://science.science |
| | GSNRASGVPDRFSGSGSGTDFTLKISR | | | | | | | | | mag.org/content/early/ |
| | VEAEDVGVYYCMQALQTPGVTFGGG | | | | | | | | | 2020/06/15/science.abc |
| | TKVDIK | | | | | | | | | 7424) |
| mAb-129 | QPVLTQPPSASGTPGQRVTISCSGSYS | IGHV4-31 | IGHJ6 | IGLV1-44 | IGLJ3 | 2913 | ARDLAKWSY | 4103 | ATWDDSL | Anna Wec et al., 2020 |
| | NIGTNPANWYQQLPGTAPKLLIYNND | (Human) | (Human) | (Human) | (Human) | | GYYYSGMDV | | NGVV | (https://science.science |
| | QRPSGVPDRFSGSKSGTSASLAISGLQ | | | | | | | | | mag.org/content/early/ |
| | SEDETDYYCATWDDSLNGVVFGGGTK | | | | | | | | | 2020/06/15/science.abc |
| | LTVL | | | | | | | | | 7424) |
| mAb-13 | DIRMTQSPLSLPVTRGQPASISCRSSH | IGHV1-69 | IGHJ6 | IGKV2-30 | IGKJ4 | 2914 | AREEYSGTVH | 4104 | MQGTDW | Anna Wec et al., 2020 |
| | NVVHSDGKTYLNWFHQRPGQAPRRL | (Human) | (Human) | (Human) | (Human) | | NFFGMDV | | PRS | (https://science.science |
| | IYQVSKRDSGVPDRFSGSGSGSDFTLTI | | | | | | | | | mag.org/content/early/ |
| | SRVEAEDVGVYYCMQGTDWPRSFGG | | | | | | | | | 2020/06/15/science.abc |
| | GTKVEIK | | | | | | | | | 7424) |
| mAb-130 | EIVLTQSPSSLSASVGDRVTITCRASRSI | IGHV3-9 | IGHJ6 | IGKV1-39 | IGKJ1 | 2915 | AKDGRYCSGI | 4105 | LHTYTTPRT | Anna Wec et al., 2020 |
| | SSYLNWYQQKPGKAPNLLIYDASTLQS | (Human) | (Human) | (Human) | (Human) | | SCRTGMDV | | | (https://science.science |
| | GVPSRFSGSGSGTDFSLTISSLQPEDFA | | | | | | | | | mag.org/content/early/ |
| | TYYCLHTYTTPRTFGQGTKVEIK | | | | | | | | | 2020/06/15/science.abc |
| | | | | | | | | | | 7424) |
| mAb-131 | EIVLTQSPATLSVSPGERITLSCRASHSV | IGHV3-48 | IGHJ3 | IGKV3-15 | IGKJ4 | 2916 | ARGLLDYLHD | 4106 | QQYNYWP | Anna Wec et al., 2020 |
| | SSNLAWYQQKPGQVPRLLIYGASARA | (Human) | (Human) | (Human) | (Human) | | AFDI | | PLT | (https://science.science |
| | TGIPARFSGSGSGTEFTLTISSLQSEDFA | | | | | | | | | mag.org/content/early/ |
| | VYYCQQYNYWPPLTFGGGTKVEIK | | | | | | | | | 2020/06/15/science.abc |
| | | | | | | | | | | 7424) |
| mAb-132 | DIQMTQSPSSLSASVGDRVTITCRASQ | IGHV4-38- | IGHJ3 | IGKV1-39 | IGKJ5 | 2917 | ASRHLDLLPIG | 4107 | QQSYRFPI | Anna Wec et al., 2020 |
| | SISTYLNWYQQKTGKAPELLIYVASSLQ | 2 | (Human) | (Human) | (Human) | | SFDV | | T | (https://science.science |
| | SGVPSRFSGSGSGTDFTLTISSLQPEDF | (Human) | | | | | | | | mag.org/content/early/ |
| | ATYYCQQSYRFPITFGQGTRLEIK | | | | | | | | | 2020/06/15/science.abc |
| | | | | | | | | | | 7424) |
| mAb-133 | EIVLTQSPATLSLSPGERATLSCRASQS | IGHV3-49 | IGHJ6 | IGKV3-11 | IGKJ3 | 2918 | TRGSGMFYG | 4108 | QQRTNWP | Anna Wec et al., 2020 |
| | VGTYLAWYQQKHGQAPRLLISDVSKR | (Human) | (Human) | (Human) | (Human) | | SSSGMDV | | GAT | (https://science.science |
| | ATGIPARFSGSGSGTDFTLTITSLEPEDF | | | | | | | | | mag.org/content/early/ |
| | AVYYCQQRTNWPGATFGPGTKVDIK | | | | | | | | | 2020/06/15/science.abc |
| | | | | | | | | | | 7424) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| mAb-134 | Ab | 229E | NL63, | | | S; Unk | B-cells; SARS-CoV1 | 813 | EVQLLESGAEVKKPGSSVKVSCKTSGGT | 1880 |
| | | (weak), | OC43 | | | | Human Patient | | FNSYTISWVRQAPGQGLEWMGGIIPIL | |
| | | HKU1 | | | | | | | DTPHYAQKFRGRVTITADKSTSTAFMDL | |
| | | (weak), | | | | | | | SSLTSEDTAVYYCAIRRDYSDYRDFDYW | |
| | | SARS-CoV1 | | | | | | | GQGTLVTVSS | |
| | | (weak), | | | | | | | | |
| | | SARS-CoV2 | | | | | | | | |
| | | (weak) | | | | | | | | |
| mAb-135 | Ab | HKU1 | 229E, | | | S; Unk | B-cells; SARS-CoV1 | 814 | EVQLVESGPGLVKPSETLSLACTVSGGSI | 1881 |
| | | (weak), | NL63, | | | | Human Patient | | SNYYWNWVRQPPGKGLEWIGNIYYSGS | |
| | | SARS-CoV1 | OC43 | | | | | | TTFNPSLKSRVTISVDTSRNQFSLKLSSVT | |
| | | (weak), | | | | | | | AADTAVYYCARQSSSWYNPYYFDQWG | |
| | | SARS-CoV2 | | | | | | | QGTLVTVSS | |
| | | (weak) | | | | | | | | |
| mAb-136 | Ab | HKU1, SARS- | 229E, | | | S; Unk | B-cells; SARS-CoV1 | 815 | QVQLVQSGGGLVQPGESLRLSCAASGF | 1882 |
| | | CoV1 | NL63, | | | | Human Patient | | NFSPYGMNWVRQAPGKGLEWIAYIISG | |
| | | (weak), | OC43 | | | | | | SGTIYYADSVKGRFTISRDNAQSSLYLQM | |
| | | SARS-CoV2 | | | | | | | NSLRAEDTAVYYCARGLLDYLHDAFDIW | |
| | | (weak) | | | | | | | GQGTMVTVSS | |
| mAb-137 | Ab | HKU1, SARS- | 229E, | | | S; Unk | B-cells; SARS-CoV1 | 816 | EVQLVESGAEVKNPGSSVKISCKSSGGTL | 1883 |
| | | CoV1 | NL63, | | | | Human Patient | | GDYAISWVRQAPGLGLEWLGGIMPLH | |
| | | (weak), | OC43 | | | | | | GTTGYSQRFRPRLTITADESARTAYMELT | |
| | | SARS-CoV2 | | | | | | | ALSSEDSAIYYCARDPSILNTGNHHWYD | |
| | | (weak) | | | | | | | LDLWGQGTEVTVSS | |
| mAb-138 | Ab | HKU1 | 229E, | | | S; Unk | B-cells; SARS-CoV1 | 817 | EVQLLESGGGVVQPGRSLRLSCAASGFT | 1884 |
| | | (weak), | NL63, | | | | Human Patient | | FNSYALFWVRQAPGKGLEWVAVVSYD | |
| | | SARS-CoV1 | OC43 | | | | | | GNNKYYADSVKGRFTISRDNSKNTLYLQ | |
| | | (weak), | | | | | | | MNSLKTEDTSVYYCARPRSGSYRQAIDY | |
| | | SARS-CoV2 | | | | | | | WGLGTLVTVSS | |
| | | (weak) | | | | | | | | |
| mAb-139 | Ab | 229E, HKU1 | OC43 | | | S; Unk | B-cells; SARS-CoV1 | 818 | QVQLVQSGGGLVQPGGSLRLSCAASGFI | 1885 |
| | | (weak), | | | | | Human Patient | | FSGHWMSWVRQAPGKGLEWVANIKQ | |
| | | NL63, SARS- | | | | | | | DGREKHYVDSVKGRFTISRDNAKNSVSL | |
| | | CoV1 | | | | | | | QMNSLRAEDTAVYYCVRQNVAIQYYYY | |
| | | (weak), | | | | | | | AMDVWGQGTTVTVSS | |
| | | SARS-CoV2 | | | | | | | | |
| | | (weak) | | | | | | | | |
| mAb-14 | Ab | SARS-CoV1, | 229E, | | | S; Unk | B-cells; SARS-CoV1 | 819 | EVQLVESGGGVVQPGRSLRLSCAASGFT | 1886 |
| | | SARS-CoV2 | HKU1, | | | | Human Patient | | FSGYPMHWVRQAPGKGLEWVALISFD | |
| | | | NL63, | | | | | | GDSKYYTDSVKARFAISRDNSKNTLFLQ | |
| | | | OC43 | | | | | | MNSLRVADTALYYCARAKGGSYSNAFD | |
| | | | | | | | | | YWGQGTLVTVSS | |
| mAb-140 | Ab | 229E, HKU1 | OC43 | | | S; Unk | B-cells; SARS-CoV1 | 820 | QVQLVESGAEVKKPGSSVKVSCKASGGT | 1887 |
| | | (weak), | | | | | Human Patient | | LSDYAISWVRQAPGQGLEWMGGIMPI | |
| | | NL63, SARS- | | | | | | | FGSPGYAEIFQGRVTITADESKSTVYMEL | |
| | | CoV1 | | | | | | | TSLRSEDTAVYYCARDPSILDTGNHHWY | |
| | | (weak), | | | | | | | DLDIWGQGTMVTVSS | |
| | | SARS-CoV2 | | | | | | | | |
| | | (weak) | | | | | | | | |
| mAb-141 | Ab | 229E, HKU1 | OC43 | | | S; Unk | B-cells; SARS-CoV1 | 821 | QVQLVQSGAEVKKPGSSVKVSCKTSGAT | 1888 |
| | | (weak), | | | | | Human Patient | | YKNSAFSWARQAPGQGFQWMGGIIPL | |
| | | NL63, SARS- | | | | | | | FGVPHYVQMFQGRVTITADESTSAVYM | |
| | | CoV1 | | | | | | | ELSGLTSDDTAVYYCAREEYSGTVHNFF | |
| | | (weak), | | | | | | | GMDVWGQGTTVTVSS | |
| | | SARS-CoV2 | | | | | | | | |
| | | (weak) | | | | | | | | |
| mAb-142 | Ab | 229E, HKU1 | OC43 | | | S; Unk | B-cells; SARS-CoV1 | 822 | EVQLVESGGGLVQPGGSLRLSCSASEFT | 1889 |
| | | (weak), | | | | | Human Patient | | LRSHAMHWVRQAPGKGLEYVSGISTDG | |
| | | NL63, SARS- | | | | | | | SGRFYADSVKGRFTISRDNSKNKLFLQM | |
| | | CoV1 | | | | | | | SSLRPEDTAVYYCVRDWGSSTHYDVFDL | |
| | | (weak), | | | | | | | WGQGTMVTVSS | |
| | | SARS-CoV2 | | | | | | | | |
| | | (weak) | | | | | | | | |
| mAb-143 | Ab | 229E, 229E | | | | S; Unk | B-cells; SARS-CoV1 | 823 | QVQLVQSGAEVKKPGSSVKVSCKASGG | 1890 |
| | | (weak), | | | | | Human Patient | | TLADYAISWVRQAPGQGLEWMGGIKP | |
| | | HKU1 | | | | | | | LHGAAGYSQHFRGRLSITADESASTAYM | |
| | | (weak), | | | | | | | ELTGLRSEDTAMYYCARDPSILNTGNHH | |
| | | NL63 | | | | | | | WYDLDLWGQGTMVTVSS | |
| | | (weak), | | | | | | | | |
| | | OC43 | | | | | | | | |
| | | (weak), | | | | | | | | |
| | | SARS-CoV1 | | | | | | | | |
| | | (weak), | | | | | | | | |
| | | SARS-CoV2 | | | | | | | | |
| | | (weak) | | | | | | | | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| mAb-134 | QPVLTQSPSASASLGGSVKLTCTLTSG | IGHV1-69 | IGHJ4 | IGLV4-69 | IGLJ3 | 2919 | AIRRDYSDYR | 4109 | HTWGTDI | Anna Wec et al., 2020 |
| | HSTYAIAWHQQQPEKGPRFLMKLNS | (Human) | (Human) | (Human) | (Human) | | DFDY | | QV | (https://science.science |
| | DGSHNKGDGIPDRFSGSSSGAERYLTI | | | | | | | | | mag.org/content/early/ |
| | SSLQSEDEADYYCHTWGTDIQVFGGG | | | | | | | | | 2020/06/15/science.abc |
| | TKLTVL | | | | | | | | | 7424) |
| mAb-135 | NFMLTQPHSVSESPGKTVTISCTRSSG | IGHV4-59 | IGHJ4 | IGLV6-57 | IGLI6 | 2920 | ARQSSSWYN | 4110 | QSYDSSSQ | Anna Wec et al., 2020 |
| | SIANNYVQWLQQRPGSSPTTIIYEDNQ | (Human) | (Human) | (Human) | (Human) | | PYYFDQ | | V | (https://science.science |
| | RPSGVPDRFSGSIDASSNSASLTISGLK | | | | | | | | | mag.org/content/early/ |
| | TEDEADYYCQSYDSSSQVFGGGTKVT | | | | | | | | | 2020/06/15/science.abc |
| | VL | | | | | | | | | 7424) |
| mAb-136 | DIVLTQSPATLSVSPGERITLSCRASHS | IGHV3-48 | IGHJ3 | IGKV3-15 | IGKJ4 | 2921 | ARGLLDYLHD | 4111 | QQYNYWP | Anna Wec et al., 2020 |
| | VSSNLAWYQQKPGQVPRLLIYGASAR | (Human) | (Human) | (Human) | (Human) | | AFDI | | PLT | (https://science.science |
| | ATGIPARFSGSGSGTEFTLTISSLQSEDF | | | | | | | | | mag.org/content/early/ |
| | AVYYCQQYNYWPPLTFGGGTKVEIK | | | | | | | | | 2020/06/15/science.abc |
| | | | | | | | | | | 7424) |
| mAb-137 | ETTLTQSPLSLSVTLGQAASISCRPNLG | IGHV1-69 | IGHJ4 | IGKV2-30 | IGKJ2 | 2922 | ARDPSILNTG | 4112 | MQGTEW | Anna Wec et al., 2020 |
| | LMHTDGNTYLNWFHQRPGQSPRRLI | (Human) | (Human) | (Human) | (Human) | | NHHWYDLDL | | PRT | (https://science.science |
| | YKVSNRDSGVPDRFAGSGSGTHFTLEI | | | | | | | | | mag.org/content/early/ |
| | SGVEADDVGVYYCMQGTEWPRTFG | | | | | | | | | 2020/06/15/science.abc |
| | QGTKLEIK | | | | | | | | | 7424) |
| mAb-138 | DIRMTQSPSTLSASVGDRVTITCRASQ | IGHV3-30 | IGHJ4 | IGKV1-5 | IGKJ2 | 2923 | ARPRSGSYRQ | 4113 | QQYNSLYT | Anna Wec et al., 2020 |
| | TIGSWLAWYQQKPGKAPNLLIYKASSL | (Human) | (Human) | (Human) | (Human) | | AIDY | | | (https://science.science |
| | ESGVPSRFSGSGSGTEFTLTISSLQPDD | | | | | | | | | mag.org/content/early/ |
| | FATYYCQQYNSLYTFGQGTKLEIK | | | | | | | | | 2020/06/15/science.abc |
| | | | | | | | | | | 7424) |
| mAb-139 | ETTLTQSPGTLSVSPGERATLSCRASQS | IGHV3-7 | IGHJ6 | IGKV3-15 | IGKJ3 | 2924 | VRQNVAIQYY | 4114 | QQYHHWP | Anna Wec et al., 2020 |
| | VISNLAWYQQKPGQAPRLLIYGASTRA | (Human) | (Human) | (Human) | (Human) | | YYAMDV | | PYT | (https://science.science |
| | TGVPARFSGSGSGTEFTLTIASLQSEDF | | | | | | | | | mag.org/content/early/ |
| | AVYYCQQYHHWPPYTFGQGTKVDIK | | | | | | | | | 2020/06/15/science.abc |
| | | | | | | | | | | 7424) |
| mAb-14 | EIVLTQSPDSLAVSLGERATINCKSSQS | IGHV3-30 | IGHJ4 | IGKV4-1 | IGKJ3 | 2925 | ARAKGGSYSN | 4115 | QQYCSTPP | Anna Wec et al., 2020 |
| | VLYSSNNENCLAWYQQKPGQPPKLLI | (Human) | (Human) | (Human) | (Human) | | AFDY | | YT | (https://science.science |
| | YWASTRESGVPDRFSGSGSGTDFTLTI | | | | | | | | | mag.org/content/early/ |
| | SSLQAEDVAVYYCQQYCSTPPYTFGQ | | | | | | | | | 2020/06/15/science.abc |
| | GTKVDIK | | | | | | | | | 7424) |
| mAb-140 | EIVMTQSPLSLPVSLGQPASISCRSSQS | IGHV1-69 | IGHJ3 | IGKV2-30 | IGKJ1 | 2926 | ARDPSILDTG | 4116 | MQGTEW | Anna Wec et al., 2020 |
| | VIHTDGNTYLNWYHQRPGQSPRRLIY | (Human) | (Human) | (Human) | (Human) | | NHHWYDLDI | | PRT | (https://science.science |
| | KVSNRDSGVPDRFSGSGSVTDFTLKIS | | | | | | | | | mag.org/content/early/ |
| | RVEAEDVGVYYCMQGTEWPRTFGQ | | | | | | | | | 2020/06/15/science.abc |
| | GTKVEIK | | | | | | | | | 7424) |
| mAb-141 | DIVMTQSPLSLPVSRGQSASISCRSSHS | IGHV1-69 | IGHJ6 | IGKV2-30 | IGKJ3 | 2927 | AREEYSGTVH | 4117 | MQGTDW | Anna Wec et al., 2020 |
| | VVHSDGKTYVNWFHQRPGQAPRRLI | (Human) | (Human) | (Human) | (Human) | | NFFGMDV | | PRS | (https://science.science |
| | YQVSKRDSGVPDRFSGSGSGFDFTLKI | | | | | | | | | mag.org/content/early/ |
| | SRVEAEDVGVYFCMQGTDWPRSFGG | | | | | | | | | 2020/06/15/science.abc |
| | GTKVDIK | | | | | | | | | 7424) |
| mAb-142 | DIVMTQSPSSLSASVGDRVTITCRASQ | IGHV3- | IGHJ3 | IGKV1-39 | IGKJ5 | 2928 | VRDWGSSTH | 4118 | QQSYSTP | Anna Wec et al., 2020 |
| | SINIYLNWYQQKPGKAPKLLIYAASSLQ | 64D | (Human) | (Human) | (Human) | | YDVFDL | | LIT | (https://science.science |
| | SGVPSRFSGSGSGTDFTLTISSLQPEDF | (Human) | | | | | | | | mag.org/content/early/ |
| | ATYYCQQSYSTPLITFGQGTRLEIK | | | | | | | | | 2020/06/15/science.abc |
| | | | | | | | | | | 7424) |
| mAb-143 | DIRLTQSPLSLPVTLGQAASISCKSSHFI | IGHV1-69 | IGHJ3 | IGKV2-30 | IGKJ3 | 2929 | ARDPSILNTG | 4119 | MQGTEW | Anna Wec et al., 2020 |
| | VHTDGNTYLNWFHQRPGQSPRRLIYK | (Human) | (Human) | (Human) | (Human) | | NHHWYDLDL | | PRT | (https://science.science |
| | VSNRDSGVPDRFSGSGSGTDFTLEISG | | | | | | | | | mag.org/content/early/ |
| | VEAEDVGVYYCMQGTEWPRTFGQGT | | | | | | | | | 2020/06/15/science.abc |
| | KVDIK | | | | | | | | | 7424) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| mAb-144 | Ab | 229E, HKU1, | OC43 | | | S; Unk | B-cells; SARS-CoV1 | 824 | QVQLVQSGAELKKPGSSVKVSCKASGG | 1891 |
| | | NL63, SARS- | | | | | Human Patient | | TFYNSAFSWLRHAPGQGPEWMGGITP | |
| | | CoV1 | | | | | | | SLGRVGYSERFLARLTITADESTSTVYME | |
| | | (weak), | | | | | | | LTSLASEDTAVYYCARDASIVGTGNHLW | |
| | | SARS-CoV2 | | | | | | | YGLDFWGHGTTVTVSS | |
| | | (weak) | | | | | | | | |
| mAb-145 | Ab | 229E, HKU1 | OC43 | | | S; Unk | B-cells; SARS-CoV1 | 825 | EVQLVESGGHVVLPGKSLRLSCAGSGFG | 1892 |
| | | (weak) | | | | | Human Patient | | FPLYAMQWVRRAPGKGLEWVALVSYD | |
| | | NL63, SARS- | | | | | | | SSNIRYADSVKGRFTISRDNSQNTLYLQ | |
| | | CoV1 | | | | | | | MDSLRPEDTAMYYCARDNALQDGRPG | |
| | | (weak), | | | | | | | YFDSWGQGTLVTVSS | |
| | | SARS-CoV2 | | | | | | | | |
| | | (weak) | | | | | | | | |
| mAb-146 | Ab | 229E, HKU1 | OC43 | | | S; Unk | B-cells; SARS-CoV1 | 826 | EVQLVESGGGLVKPGGSLRLSCAASGFIF | 1893 |
| | | (weak), | | | | | Human Patient | | SDYYMSWIRQAPGKGLEWVSYITGSGR | |
| | | NL63, SARS- | | | | | | | TIHYADSVKGRFTISRDNAKNSVYLQMN | |
| | | CoV1 | | | | | | | SLRAEDTAVYYCARGHRFLEFPLNYFDP | |
| | | (weak), | | | | | | | WGQGTLVTVSS | |
| | | SARS-CoV2 | | | | | | | | |
| | | (weak) | | | | | | | | |
| mAb-147 | Ab | 229E, HKU1 | OC43 | | | S; Unk | B-cells; SARS-CoV1 | 827 | EVQLLESGGHVVLPGKSLRLSCAGSGFG | 1894 |
| | | (weak), | | | | | Human Patient | | FPLYAMQWVRRAPGKGLEWVALVSYD | |
| | | NL63 | | | | | | | SSNIRYADSVKGRFTISRDNSQNTLYLQ | |
| | | (weak), | | | | | | | MDSLRPEDTAMYYCARDNALQDGRPG | |
| | | SARS-CoV1 | | | | | | | YFDSWGQGTLVTVSS | |
| | | (weak), | | | | | | | | |
| | | SARS-CoV2 | | | | | | | | |
| | | (weak) | | | | | | | | |
| mAb-148 | Ab | 229E, HKU1 | OC43 | | | S; Unk | B-cells; SARS-CoV1 | 828 | EVQLVESGGGVVQPGRSLRLSCAASGFT | 1895 |
| | | (weak), | | | | | Human Patient | | FSKFAMHWVRQAPGKGLEWVAIISYDG | |
| | | NL63 | | | | | | | SHKNYADSVKGRFTISRDNSKNTVYLQV | |
| | | (weak), | | | | | | | DSLRAEDTAVYYCALLYGSGSYYNFVFFG | |
| | | SARS-CoV1 | | | | | | | WKDGSDAWGPGTTVTVSS | |
| | | (weak), | | | | | | | | |
| | | SARS-CoV2 | | | | | | | | |
| | | (weak) | | | | | | | | |
| mAb-149 | Ab | 229E | OC43 | | | S; Unk | B-cells; SARS-CoV1 | 829 | QVQLVQSGAEVKKPGSSVKVSCKASGG | 1896 |
| | | (weak), | | | | | Human Patient | | MFTDYAISWVRQAPGQRLEWMGGILP | |
| | | HKU1, NL63 | | | | | | | AFAASGSPGYAPIFRGRATFSADVSTSTA | |
| | | (weak), | | | | | | | YLELTNLKPEDTAVYYCARDPSILNTGNH | |
| | | SARS-CoV1 | | | | | | | HWYDLDLWGQGTEVTVSS | |
| | | (weak), | | | | | | | | |
| | | SARS-CoV2 | | | | | | | | |
| | | (weak) | | | | | | | | |
| mAb-15 | Ab | SARS-CoV1, | 229E, | | | S; Unk | B-cells; SARS-Cov1 | 830 | QVQLVQSGAELKKPGSSVRVSCKAAGG | 1897 |
| | | SARS-CoV2 | HKU1, | | | | Human Patient | | TLTNYAISWVRQAPGQGFEWMGGIMP | |
| | | | NL63, | | | | | | VSHTAGYAQKFQGRVTFTADESATTAY | |
| | | | OC43 | | | | | | MDLTSLRPEDTAIYYCARDPSIHYTGNH | |
| | | | | | | | | | HWYDLDIWGQGTMVTVSS | |
| mAb-150 | Ab | 229E | OC43 | | | S; Unk | B-cells; SARS-CoV1 | 831 | QVQLVQSGAELKKPGSSVRVSCKAAGG | 1898 |
| | | (weak), | | | | | Human Patient | | TLTNYAISWVRQAPGQGFEWMGGIMP | |
| | | HKU1 | | | | | | | VSHTAGYAQKFQGRVTFTADESATTAY | |
| | | (weak), | | | | | | | MDLTSLRPEDTAIYYCARDPSIHYTGNH | |
| | | NL63, SARS- | | | | | | | HWYDLDIWGQGTMVTVSS | |
| | | CoV1 | | | | | | | | |
| | | (weak), | | | | | | | | |
| | | SARS-CoV2 | | | | | | | | |
| | | (weak) | | | | | | | | |
| mAb-151 | Ab | 229E | OC43 | | | S; Unk | B-cells; SARS-CoV1 | 832 | QVQLVQSGAEVKKPGSSMRVSCRVSG | 1899 |
| | | (weak), | | | | | Human Patient | | GTFITHAMSWVRQAPGQGPEWMGGI | |
| | | HKU1 | | | | | | | VPLFGRASYAQPSQTRVQITADESTSTVY | |
| | | (weak), | | | | | | | LEVPSLTSEDTAVYYCVRDSEPYTATRSQ | |
| | | NL63 | | | | | | | NHYWYDMDVWGQGTTVTVSS | |
| | | (weak), | | | | | | | | |
| | | SARS-CoV1 | | | | | | | | |
| | | (weak), | | | | | | | | |
| | | SARS-CoV2 | | | | | | | | |
| | | (weak) | | | | | | | | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| mAb-144 | ETTLTQSPLFLPVTLGQPASISCTSSVH | IGHV1-69 | IGHJ6 | IGKV2-30 | IGKJ3 | 2930 | ARDASIVGTG | 4120 | MQGTDW | Anna Wec et al., 2020 |
| | VVHSDGNTYLNWFQQRPGQSPRRLIY | (Human) | (Human) | (Human) | (Human) | | NHLWYGLDF | | PRT | (https://science.science |
| | KVSNRDSGVPDRFSGSGSGTYFTLKIS | | | | | | | | | mag.org/content/early/ |
| | RVEAEDVGVYYCMQGTDWPRTFGQ | | | | | | | | | 2020/06/15/science.abc |
| | GTKVDIK | | | | | | | | | 7424) |
| mAb-145 | ETTLTQSPATLSLSPGERATLSCRASQSI | IGHV3-30 | IGHJ5 | IGKV3-11 | IGKJ3 | 2931 | ARDNALQDG | 4121 | QQRFSWY | Anna Wec et al., 2020 |
| | NDYLGWYQHRPGQAPRLLIHDASTRA | (Human) | (Human) | (Human) | (Human) | | RPGYFDS | | N | (https://science.science |
| | PGIPVRFSGSGSGTDFTLTISSLEPEDSA | | | | | | | | | mag.org/content/early/ |
| | VYYCQQRFSWYNFGPGTKVEIK | | | | | | | | | 2020/06/15/science.abc |
| | | | | | | | | | | 7424) |
| mAb-146 | QSVLTQPASVSGSPGQSITISCAVTSSD | IGHV3-11 | IGHJ5 | IGLV2-23 | IGLJ3 | 2932 | ARGHRFLEFP | 4122 | CSYGGRST | Anna Wec et al., 2020 |
| | VGSYNLVSWHQQHPGKAPKLMIYEV | (Human) | (Human) | (Human) | (Human) | | LNYFDP | | SVI | (https://science.science |
| | NKRPSGVSNRFSGSKSGNTASLTISGL | | | | | | | | | mag.org/content/early/ |
| | QAEDEAVYYCCSYGGRSTSVVFGGGT | | | | | | | | | 2020/06/15/science.abc |
| | KLTVL | | | | | | | | | 7424) |
| mAb-147 | DIRLTQSPATLSLSPGERATLSCRASQSI | IGHV3-30 | IGHJ5 | IGKV3-11 | IGKJ2 | 2933 | ARDNALQDG | 4123 | QQRFSWY | Anna Wec et al., 2020 |
| | NDYLGWYQHRPGQAPRLLIHDASTRA | (Human) | (Human) | (Human) | (Human) | | RPGYFDS | | N | (https://science.science |
| | PGIPVRFSGSGSGTDFTLTISSLEPEDSA | | | | | | | | | mag.org/content/early/ |
| | VYYCQQRFSWYNFGPGTKLEIK | | | | | | | | | 2020/06/15/science.abc |
| | | | | | | | | | | 7424) |
| mAb-148 | QPVLTQPASVSGSPGQSITISCTGTTSD | IGHV3-30 | IGHJ6 | IGLV2-14 | IGLJ3 | 2934 | ALLYGSGSYY | 4124 | SSYTSGGTL | Anna Wec et al., 2020 |
| | VGGYDYVSWYQQRPGKAPKLIIYDVI | (Human) | (Human) | (Human) | (Human) | | NFVFFGWKD | | V | (https://science.science |
| | NRPSGVSNRFSGSKSGNTASLTISGLQ | | | | | | GSDA | | | mag.org/content/early/ |
| | ADDETDYYCSSYTSGGTLVFGTGTKLT | | | | | | | | | 2020/06/15/science.abc |
| | VL | | | | | | | | | 7424) |
| mAb-149 | DIQMTQSPLSLPVGLGQSASISCRSSQ | IGHV1-69 | IGHJ4 | IGKV2-30 | IGKJ1 | 2935 | ARDPSILNTG | 4125 | MQATEWP | Anna Wec et al., 2020 |
| | WVVHTDGNTYLNWFHQRPGQSPRR | (Human) | (Human) | (Human) | (Human) | | NHHWYDLDL | | RT | (https://science.science |
| | LIYKVSNRDSGVPDRFSGSGSGTDFTL | | | | | | | | | mag.org/content/early/ |
| | RISRVEAEDVGVYYCMQATEWPRTFG | | | | | | | | | 2020/06/15/science.abc |
| | QGTKVEIK | | | | | | | | | 7424) |
| mAb-15 | DIRLTQSPLSLPVTLGQPASISCRSSQRI | IGHV1-69 | IGHJ3 | IGKV2-30 | IGKJ3 | 2936 | ARDPSIHYTG | 4126 | MQGTEW | Anna Wec et al., 2020 |
| | VHTDGNTYLNWFLQRPGQSPRRLIYK | (Human) | (Human) | (Human) | (Human) | | NHHWYDLDI | | PRT | (https://science.science |
| | VSNRDSGVPDRFSGSGSGTDFTLKISR | | | | | | | | | mag.org/content/early/ |
| | VEAEDVGIYYCMQGTEWPRTFGQGT | | | | | | | | | 2020/06/15/science.abc |
| | KVDIK | | | | | | | | | 7424) |
| mAb-150 | DIQLTQSPLSLPVTLGQPASISCRSSQRI | IGHV1-69 | IGHJ3 | IGKV2-30 | IGKJ1 | 2937 | ARDPSIHYTG | 4127 | MQGTEW | Anna Wec et al., 2020 |
| | VHTDGNTYLNWFLQRPGQSPRRLIYK | (Human) | (Human) | (Human) | (Human) | | NHHWYDLDI | | PRT | (https://science.science |
| | VSNRDSGVPDRFSGSGSGTDFTLKISR | | | | | | | | | mag.org/content/early/ |
| | VEAEDVGIYYCMQGTEWPRTFGQGT | | | | | | | | | 2020/06/15/science.abc |
| | KVEIK | | | | | | | | | 7424) |
| mAb-151 | ETTLTQSPLSLSVTLGQSASISCRASQT | IGHV1-69 | (Human) | IGKV2-30 | IGKJ1 | 2938 | VRDSEPYTAT | 4128 | MQGTDW | Anna Wec et al., 2020 |
| | VVHSVDGNTYLNWFHQRPGQSPRRLI | (Human) | IGHJ6 | (Human) | (Human) | | RSQNHYWYD | | PRT | (https://science.science |
| | YKVSNRDSGVPDRFSGSGSGTDFTLRI | | | | | | MDV | | | mag.org/content/early/ |
| | SRVEAEDIGIYYCMQGTDWPRTFGQG | | | | | | | | | 2020/06/15/science.abc |
| | TKVEIK | | | | | | | | | 7424) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| mAb-152 | Ab | 229E | OC43 | | | S; Unk | B-cells; SARS-CoV1 | 833 | QVQLVQSGAELKKPGSSVRVSCKAAGG | 1900 |
| | | (weak), | | | | | Human Patient | | TLTNYAISWVRQAPGQGFEWMGGIMP | |
| | | HKU1 | | | | | | | VSHTAGYAQKFQGRVTFTADESATTAY | |
| | | (weak), | | | | | | | MDLTSLRPEDTAIYYCARDPSIHYTGNH | |
| | | NL63 | | | | | | | HWYDLDIWGQGTTVTVSS | |
| | | (weak), | | | | | | | | |
| | | SARS-CoV1 | | | | | | | | |
| | | (weak), | | | | | | | | |
| | | SARS-CoV2 | | | | | | | | |
| | | (weak) | | | | | | | | |
| mAb-153 | Ab | 229E | OC43 | | | S; Unk | B-cells; SARS-CoV1 | 834 | QVQLVQSGAEVKKPGSSVKVSCKASGG | 1901 |
| | | (weak), | | | | | Human Patient | | TLSHYAISWVRQAPGQGLEWMGGIMP | |
| | | HKU1 | | | | | | | VSGTVGYAQKFQGRVKFTADEYASTAY | |
| | | (weak), | | | | | | | MELTNLRSEDSAVYFCARDPSIVDSGPH | |
| | | NL63 | | | | | | | HWYDLDIWGQGTMVTVSS | |
| | | (weak), | | | | | | | | |
| | | SARS-CoV1 | | | | | | | | |
| | | (weak), | | | | | | | | |
| | | SARS-CoV2 | | | | | | | | |
| | | (weak) | | | | | | | | |
| mAb-154 | Ab | 229 | OC43 | | | S; Unk | B-cells; SARS-CoV1 | 835 | QVQLVESGAEVKRPGASVKVSCKASGY | 1902 |
| | | (weak), | | | | | Human Patient | | AFSDYYMHWVRQAPGQGPEWMGWI | |
| | | HKU1 | | | | | | | NPNSGDTGYPQKFRGWVTMTRDTSVS | |
| | | (weak), | | | | | | | TAYMELKRLKSDDTAVYYCASGPNYFDY | |
| | | NL63 | | | | | | | WGQGTLVTVSS | |
| | | (weak), | | | | | | | | |
| | | SARS-CoV1 | | | | | | | | |
| | | (weak), | | | | | | | | |
| | | SARS-CoV2 | | | | | | | | |
| | | (weak) | | | | | | | | |
| mAb-155 | Ab | 229E | OC43 | | | S; Unk | B-cells; SARS-CoV1 | 836 | EVQLLESGGGLVKPGGSLRLSCEASGFTF | 1903 |
| | | (weak), | | | | | Human Patient | | SNYNMNWVRQAPGKGLEWVSSISGSS | |
| | | HKU1 | | | | | | | SYISYADSVKGRFTISRDNAKNSLYLQMN | |
| | | (weak), | | | | | | | SLRVEDTAVYYCARADRDYDFWSDPPLI | |
| | | NL63 | | | | | | | DHWGQGTLVTVSS | |
| | | (weak), | | | | | | | | |
| | | SARS-CoV1 | | | | | | | | |
| | | (weak), | | | | | | | | |
| | | SARS-CoV2 | | | | | | | | |
| | | (weak) | | | | | | | | |
| mAb-156 | Ab | 229 | NL63, | | | S; Unk | B-cells; SARS-CoV1 | 837 | EVQLVESGGGLIQPGGSLRLSCAASGFT | 1904 |
| | | (weak), | OC43 | | | | Human Patient | | VTDNYMSWVRQAPGKGLEWVSVLYSG | |
| | | HKU1 | | | | | | | GSTYYADAVQGRFSISRDNSKNALYLQM | |
| | | (weak), | | | | | | | NSLRAEDTAVYYCARGFGNGWSYYFDY | |
| | | SARS-CoV1 | | | | | | | WGQGTLVTVSS | |
| | | (weak), | | | | | | | | |
| | | SARS-CoV2 | | | | | | | | |
| | | (weak) | | | | | | | | |
| mAb-157 | Ab | HKU1, SARS- | 229E, | | | S; Unk | B-cells; SARS-CoV1 | 838 | EVQLVQSGGGLVQPGGSLRLSCAASGF | 1905 |
| | | CoV1 | NL63, | | | | Human Patient | | TFSSYSMSWVRQAPGKGLEWVSYITRSS | |
| | | (weak), | OC43 | | | | | | DNIYYAESVKGRFTISRDSAKNSLYLHMN | |
| | | SARS-CoV2 | | | | | | | SLRDEDTAVYYCARDPGLEYSGNYFSYYY | |
| | | (weak) | | | | | | | YAMDVWGQGTTVTVSS | |
| mAb-158 | Ab | HKU1, SARS- | 229E, | | | S; Unk | B-cells; SARS-CoV1 | 839 | QVQLVQSGAEVKKPGSSVKVSCKASGG | 1906 |
| | | CoV1 | NL63, | | | | Human Patient | | TFSTHAISWVRQAPGHGPEWMGGIIPL | |
| | | (weak), | OC43 | | | | | | FGTSESAQRFQARVRFTADESTSTAYME | |
| | | SARS-CoV2 | | | | | | | LSSLTSEDTAVYYCVRDSDPYTATSRNNH | |
| | | (weak) | | | | | | | YWYDMDVWGQGTTVTVSS | |
| mAb-159 | Ab | HKU1, SARS- | 229E, | | | S; Unk | B-cells; SARS-CoV1 | 840 | QVQLVQSGAEVKKPGSSVKVSCKVSGG | 1907 |
| | | CoV1 | NL63, | | | | Human Patient | | RFSDYAISWLRQAPVKGLEWMGGIIPRL | |
| | | (weak), | OC43 | | | | | | NRKGYSQDFQGRLTFTADESTSTAYMEL | |
| | | SARS-CoV2 | | | | | | | SGLTSEDTAVYYCARDPTFLNSGNHFWY | |
| | | (weak) | | | | | | | AVDIWGQGTTVTVSS | |
| mAb-16 | Ab | SARS-CoV1, | 229E, | | | S; Unk | B-cells; SARS-CoV1 | 841 | EVQLVESGAEVRTPGSSVKLSCKASGGT | 1908 |
| | | SARS-CoV2 | HKU1, | | | | Human Patient | | FSTHAFSWVRQAPGQRPEWMGGIIPIF | |
| | | | NL63, | | | | | | GESKDTQKFQGRVTFTADESTTTVYMEL | |
| | | | OC43 | | | | | | RSLKSDDTAIYYCVRDSDPYTATYRNNHY | |
| | | | | | | | | | WYAMDVWGQGTTVTVSS | |
| mAb-160 | Ab | HKU1, SARS- | 229E, | | | S; Unk | B-cells; SARS-CoV1 | 842 | EVQLVESGGGVVKPGGSLRVSCVGSGF | 1909 |
| | | CoV1 | NL63, | | | | Human Patient | | TFSDHYMSWIRQAPGKGLEIISYISTDGS | |
| | | (weak), | OC43 | | | | | | YINDADSVKGRFINSRDNAKNSVYLQLN | |
| | | SARS-CoV2 | | | | | | | SLRAEDTAVYYCARMGPSGSGSLDYWG | |
| | | (weak) | | | | | | | QGSLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| mAb-152 | DIVMTQSPLSLPVTLGQPASISCRSSQR | IGHV169 | IGHJ6 | IGKV2-30 | IGKJ1 | 2939 | ARDPSIHYTG | 4129 | MQGTEW | Anna Wec et al., 2020 |
| | IVHTDGNTYLNWFLQRPGQSPRRLIYK | (Human) | (Human) | (Human) | (Human) | | NHHWYDLDI | | PRT | (https://science.science |
| | VSNRDSGVPDRFSGSGSGTDFTLKISR | | | | | | | | | mag.org/content/early/ |
| | VEAEDVGIYYCMQGTEWPRTFGQGT | | | | | | | | | 2020/06/15/science.abc |
| | KVEIK | | | | | | | | | 7424) |
| mAb-153 | DIRMTQSPLSLPVTLGQPASISCRSSQF | IGHV1-69 | IGHJ3 | IGKV2-30 | IGKJ3 | 2940 | ARDPSIVDSG | 4130 | MQATEWP | Anna Wec et al., 2020 |
| | VVHTDGNTYLNWFQQRPGQSPRRLIY | (Human) | (Human) | (Human) | (Human) | | PHHWYDLDI | | RT | (https://science.science |
| | KVSNRDSGVPDRFSGSGSGTDFTLKIS | | | | | | | | | mag.org/content/early/ |
| | RVEAEDVGVYYCMQATEWPRTFGQG | | | | | | | | | 2020/06/15/science.abc |
| | TKVDIK | | | | | | | | | 7424) |
| mAb-154 | SYVLTQPPSVSVSPGQTARITCSADGL | IGHV1-2 | IGHJ4 | IGLV3-25 | IGLJ7 | 2941 | ASGPNYFDY | 4131 | QSADSNDS | Anna Wec et al., 2020 |
| | PKQYSYWYQQKPGQAPVMVIYKDTE | (Human) | (Human) | (Human) | (Human) | | | | SPV | (https://science.science |
| | RPSGIPERFSGSSSGTTATLTISGVQAE | | | | | | | | | mag.org/content/early/ |
| | DEADYYCQSADSNDSSPVFGGGTQLT | | | | | | | | | 2020/06/15/science.abc |
| | VL | | | | | | | | | 7424) |
| mAb-155 | ETTLTQSPATLSLSPGEGATLSCRASQS | IGHV3-21 | IGHJ4 | IGKV3-11 | IGKJ1 | 2942 | ARADRDYDF | 4132 | HQRSNWP | Anna Wec et al., 2020 |
| | VSSSLAWYQQKPGQAPRLLIYDASNR | (Human) | (Human) | (Human) | (Human) | | WSDPPLIDH | | YT | (https://science.science |
| | ATGIPARFSGSGSGTDYTLTISSLEPEDF | | | | | | | | | mag.org/content/early/ |
| | AVYYCHQRSNWPYTFGQGTKVEIK | | | | | | | | | 2020/06/15/science.abc |
| | | | | | | | | | | 7424) |
| mAb-156 | DIVLTQTPSSLSASVGDRVTITCRASQTI | IGHV3-53 | IGHJ4 | IGKV1-39 | IGKJ1 | 2943 | ARGFGNGWS | 4133 | QQSYSIPW | Anna Wec et al., 2020 |
| | ATYLSWYQQKPGKAPKLLIYAVSSLQS | (Human) | (Human) | (Human) | (Human) | | YYFDY | | T | (https://science.science |
| | GVPSRFSGSGSGTDFTLTIGSLQPEDF | | | | | | | | | mag.org/content/early/ |
| | ATYYCQQSYSIPWTFGQGTKVDIK | | | | | | | | | 2020/06/15/science.abc |
| | | | | | | | | | | 7424) |
| mAb-157 | EIVMTQSPSSLSASVGDRVTITCRASQS | IGHV3-48 | IGHJ6 | IGKV1-39 | IGKJ4 | 2944 | ARDPGLEYSG | 4134 | QHSYSSP | Anna Wec et al., 2020 |
| | ISYYLNWYQQKPGKAPKLLIYAASSLQS | (Human) | (Human) | (Human) | (Human) | | NYFSYYYYAM | | PLT | (https://science.science |
| | GVPSRFSGSGSGTDFILTISSLQPEDIAT | | | | | | DV | | | mag.org/content/early/ |
| | YYCQHSYSSPPLTFGGGTKVEIK | | | | | | | | | 2020/06/15/science.abc |
| | | | | | | | | | | 7424) |
| mAb-158 | ETTLTQSPLSLPVTLGQPASISCRSSQIV | IGHV1-69 | IGHJ6 | IGKV2-30 | IGKJ3 | 2945 | VRDSDPYTAT | 4135 | MQGTDW | Anna Wec et al., 2020 |
| | VHSDGNTYLNWFHQRPGQSPRRLIYK | (Human) | (Human) | (Human) | (Human) | | SRNNHYWYD | | PRT | (https://science.science |
| | VSNRDSGVPDRFSGSGSGTDFTLKISR | | | | | | MDV | | | mag.org/content/early/ |
| | VEAEDIGVYYCMQGTDWPRTFGQGT | | | | | | | | | 2020/06/15/science.abc |
| | KVDIK | | | | | | | | | 7424) |
| mAb-159 | DIRLTQSPLSLSVTLGQAASISCTCSQT | IGHV1-69 | IGHJ6 | IGKV2-30 | IGKJ1 | 2946 | ARDPTFLNSG | 4136 | MQTTDWP | Anna Wec et al., 2020 |
| | AVHSDGNTYLNWFHQRPGQSPRRLIY | (Human) | (Human) | (Human) | (Human) | | NHFWYAVDI | | RT | (https://science.science |
| | KVSNRDSGVPDRFSGSGSGTDFTLKIS | | | | | | | | | mag.org/content/early/ |
| | RVEAEDVGIYYCMQTTDWPRTFGQG | | | | | | | | | 2020/06/15/science.abc |
| | TKVEIK | | | | | | | | | 7424) |
| mAb-16 | EIVLTQSPLSLPVTLGQPASISCRSSLRL | IGHV1-69 | IGHJ6 | IGKV2-30 | IGKJ3 | 2947 | VRDSDPYTAT | 4137 | MQGTEW | Anna Wec et al., 2020 |
| | VHTDGNTYLNWFQQRPGQSPRRLIYK | (Human) | (Human) | (Human) | (Human) | | YRNNHYWYA | | PRT | (https://science.science |
| | VSNRDSGVPDRFSGSGSGTDFTLKISR | | | | | | MDV | | | mag.org/content/early/ |
| | VEAEDIGVYYCMQGTEWPRTFGQGT | | | | | | | | | 2020/06/15/science.abc |
| | KVDIK | | | | | | | | | 7424 |
| mAb-160 | DIVMTQSPGTLSLSPGERAALSCRASQ | IGHV3-11 | IGHJ4 | IGKV3-20 | IGKJ3 | 2948 | ARMGPSGSG | 4138 | HQYSGSAT | Anna Wec et al., 2020 |
| | IVTRSQLAWYQHKPGQPPRLLIYDSSS | (Human) | (Human) | (Human) | (Human) | | SLDY | | | (https://science.science |
| | RATGTPDRFSGSGSGTDFTLTISRLEPE | | | | | | | | | mag.org/content/early/ |
| | DSAVYYCHQYSGSATFGPGTKVEIK | | | | | | | | | 2020/06/15/science.abc |
| | | | | | | | | | | 7424) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| mAb-161 | Ab | HKU1, SARS- | 229E, | | | S; Unk | B-cells; SARS-CoV1 | 843 | EVQLVESGPTLVKPTQTLTLTCTFSGFSL | 1910 |
| | | CoV1 | NL63, | | | | Human Patient | | NTRELGVGWIRQPPGKALEWLALIYWD | |
| | | (weak), | OC43 | | | | | | DDKRYSPSLKSRLSITKDTSKNQVVLTLT | |
| | | SARS-CoV2 | | | | | | | NMDPGDTATYYCAHTSELPPRRPYAAF | |
| | | (weak) | | | | | | | DFWGQGTLVTVSS | |
| mAb-162 | Ab | HKU1 | 229E, | | | S; Unk | B-cells; SARS-CoV1 | 844 | EVQLVESGGGVVQPGGSLRLSCVASGF | 1911 |
| | | (weak), | NL63, | | | | Human Patient | | PFGRYAMHWVRQAPGQGLEWLTLISF | |
| | | SARS-CoV1 | OC43 | | | | | | DSSNIEYSDSVQGRFTISRDNSRNTLFLQ | |
| | | (weak), | | | | | | | MTSLRPEDTAVYFCARDLPPLDYWGQG | |
| | | SARS-CoV2 | | | | | | | TLVTVSS | |
| | | (weak) | | | | | | | | |
| mAb-163 | Ab | HKU1 | 229E, | | | S; Unk | B-cells; SARS-CoV1 | 845 | QVQLQQWGAGLLKPSETLSLTCAVNGG | 1912 |
| | | (weak), | OC43 | | | | Human Patient | | SFNNYYWSWIRQPPGKGPEWIGEVVHS | |
| | | NL63 | | | | | | | GSTTYNPSLKSRVTISIDMSKNQFALKLN | |
| | | (weak), | | | | | | | SVTAADTAVYYCARGFTFTYSDFLTGQRT | |
| | | SARS-CoV1 | | | | | | | FEYWGQGTLVTVSS | |
| | | (weak), | | | | | | | | |
| | | SARS-CoV2 | | | | | | | | |
| | | (weak) | | | | | | | | |
| mAb-164 | Ab | HKU1 | 229E, | | | S; Unk | B-cells; SARS-CoV1 | 846 | EVQLVESGGGVVQPGRSLRLSCAASGFT | 1913 |
| | | (weak), | OC43 | | | | Human Patient | | FSSYAMHWVRQAPGKGLEWVALISYD | |
| | | NL63 | | | | | | | GDKKYYPDSVRGRFTISRDNSKNTLHLQ | |
| | | (weak), | | | | | | | MNSLRLEDTAVYYCARSYGGSYSTVGY | |
| | | SARS-CoV1 | | | | | | | WGQGALVTVSS | |
| | | (weak), | | | | | | | | |
| | | SARS-CoV2 | | | | | | | | |
| | | (weak) | | | | | | | | |
| mAb-165 | Ab | HKU1 | 229E, | | | S; Unk | B-cells; SARS-CoV1 | 847 | QVQLVQSGGDSVNPGGSLRLSCAGSGF | 1914 |
| | | (weak), | OC43 | | | | Human Patient | | SVRDVWMSWVRQAPGKGLEWIGRIKS | |
| | | NL63 | | | | | | | EADGGSSDYRASLKDRFSIWRDASKNTL | |
| | | (weak), | | | | | | | YLQVNGLQTEDTAIYFCSWNDVGWAFT | |
| | | SARS-CoV1 | | | | | | | FWGQGTLVTVSS | |
| | | (weak), | | | | | | | | |
| | | SARS-CoV2 | | | | | | | | |
| | | (weak) | | | | | | | | |
| mAb-166 | Ab | HKU1 | 229E, | | | S; Unk | B-cells; SARS-CoV1 | 848 | QVQLVQSGAEVKKAGSSVKVSCKASGG | 1915 |
| | | (weak), | NL63, | | | | Human Patient | | PFSSFAISWVRQAPGQGLEWLGGIMTV | |
| | | SARS-CoV1 | OC43 | | | | | | FGPAHYAQKSRDRISITADESTSTSYLELS | |
| | | (weak), | | | | | | | SLTSDDTAVYYCAAEERSGTNHNYYGLD | |
| | | SARS-CoV2 | | | | | | | VWGQGTTVTVSS | |
| | | (weak) | | | | | | | | |
| mAb-167 | Ab | HKU1 | 229E, | | | S; Unk | B-cells; SARS-CoV1 | 849 | EVQLVESGGGLIQPGGSLRLSCAASGFP | 1916 |
| | | (weak), | NL63, | | | | Human Patient | | FSGTYMTWVRQAPGKGLEWVSIIYSGG | |
| | | SARS-CoV1 | OC43 | | | | | | DTYYADSVKGRFTISRDNSKNTLFLQMN | |
| | | (weak), | | | | | | | SLRVEDTAMYYCARDREMAIITERSYGL | |
| | | SARS-CoV2 | | | | | | | DVWGQGTMVTVSS | |
| | | (weak) | | | | | | | | |
| mAb-168 | Ab | HKU1 | 229E, | | | S; Unk | B-cells; SARS-CoV1 | 850 | EVQLVESGGGLVQPGGSLRLSCGASGFT | 1917 |
| | | (weak), | NL63, | | | | Human Patient | | FSDYAMSWVRQAPGKGLEWVSTISGSG | |
| | | SARS-CoV1 | OC43 | | | | | | DKTYYADSLKGRFTNSRDNSKSTLYLQM | |
| | | (weak), | | | | | | | TSLRAEDTAVYFCAKDRYCSGGSCFYDAF | |
| | | SARS-CoV2 | | | | | | | DIWGQGTTVTVSS | |
| | | (weak) | | | | | | | | |
| mAb-169 | Ab | HKU1 | 229E, | | | S; Unk | B-cells; SARS-CoV1 | 851 | EVQLVESGGGVVQPGRSLRLSCEASGFT | 1918 |
| | | (weak), | NL63, | | | | Human Patient | | FADYPMHWVRQAPGKGLEWVAVISSH | |
| | | SARS-CoV1 | OC43 | | | | | | GRSQGYAASVKGRFTFSRDNSQSSLFLQ | |
| | | (weak), | | | | | | | LNSLRVEDTAVYFCAREAQSSGRAGCLD | |
| | | SARS-CoV2 | | | | | | | AWGQGTLVTVSS | |
| | | (weak) | | | | | | | | |
| mAb-17 | Ab | SARS-CoV1, | 229E, | | | S; Unk | B-cells; SARS-CoV1 | 852 | QVQLVQSGAELKKPGSSVRVSCKAAGG | 1919 |
| | | SARS-CoV2 | HKU1, | | | | Human Patient | | TLTNYAISWVRQAPGQGFEWMGGIMP | |
| | | | NL63, | | | | | | VSHTAGYAQKFQGRVTFTADESATTAY | |
| | | | OC43 | | | | | | MDLTSLRPEDTAIYYCARDPSIHYTGNH | |
| | | | | | | | | | HWYDLDIWGQGTMVTVSS | |
| mAb-170 | Ab | HKU1 | 229E, | | | S; Unk | B-cells; SARS-CoV1 | 853 | QVQLVQSGAEVREPGSSVKLSCKTSGGT | 1920 |
| | | (weak), | NL63, | | | | Human Patient | | FSTHAISWVRQAPGQRPEWMGGIMPI | |
| | | SARS-CoV1 | OC43 | | | | | | FGESKDTQKFQGRVTFTADESTTTAYME | |
| | | (weak), | | | | | | | LRSLKSDDTAIYYCVRDSDPYTATVRSNH | |
| | | SARS-CoV2 | | | | | | | YWYAMDVWGQGTTVTVSS | |
| | | (weak) | | | | | | | | |
| mAb-171 | Ab | HKU1 | 229E, | | | S; Unk | B-cells; SARS-CoV1 | 854 | EVQLVQSGAEVKKPGSSVKVSCKVSGG | 1921 |
| | | (weak), | NL63, | | | | Human Patient | | RFSDYAISWLRQAPVKGLEWMGGIIPRL | |
| | | SARS-CoV1 | OC43 | | | | | | NRKGYSQDFQGRLTFTADESTSTAYMEL | |
| | | (weak), | | | | | | | SGLTSEDTAVYYCARDPTFLNSGNHFWY | |
| | | SARS-CoV2 | | | | | | | AVDIWGQGTTVTVSS | |
| | | (weak) | | | | | | | | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| mAb-161 | SYELTQPPSVSGAPRQKVTISCSGSSAN | IGHV2-5 | IGHJ4 | IGLV1-36 | IGLJ3 | 2949 | AHTSELPPRR | 4139 | ATWDDILN | Anna Wec et al., 2020 |
| | IASNGVNWYQQLPGKAPKLLIYYDDL | (Human) | (Human) | (Human) | (Human) | | PYAAFDF | | GPV | (https://science.science |
| | VSSGVSDRFSGSKSGTSASLAISGLQSE | | | | | | | | | mag.org/content/early/ |
| | DEADYYCATWDDILNGPVFGGGTKLT | | | | | | | | | 2020/06/15/science.abc |
| | VL | | | | | | | | | 7424) |
| mAb-162 | DIRMTQSPSSLSASVGDRVTITCRASQ | IGHV3-30 | IGHJ4 | IGKV1-27 | IGKJ1 | 2950 | ARDLPPLDY | 4140 | QKYNRAP | Anna Wec et al., 2020 |
| | GFGNKVAWYQQKPGTAPKLLIYETSTL | (Human) | (Human) | (Human) | (Human) | | | | WT | (https://science.science |
| | QSGVPSRFSGSGSGTEFAFTISSLQPED | | | | | | | | | mag.org/content/early/ |
| | GATYYCQKYNRAPWTFGQGTKVEIK | | | | | | | | | 2020/06/15/science.abc |
| | | | | | | | | | | 7424) |
| mAb-163 | QSALIQPASVSGSPGQSITISCTGTSSD | IGHV4-34 | IGHJ4 | IGLV2-14 | IGLJ3 | 2951 | ARGFTFTYSD | 4141 | SSYISDIKLV | Anna Wec et al., 2020 |
| | VGRYNYVSWYQQHPGKAPKLMIYDV | (Human) | (Human) | (Human) | (Human) | | FLTGQRTFEY | | V | (https://science.science |
| | SNRPSGVSNRFSGSKSGNTASLTISGL | | | | | | | | | mag.org/content/early/ |
| | QAEDEAAYYCSSYISDIKLVVFGGGTKL | | | | | | | | | 2020/06/15/science.abc |
| | TVL | | | | | | | | | 7424) |
| mAb-164 | QSALIQPRSVSGSPGQSVTISCTGTSSD | IGHV3-30 | IGHJ4 | IGLV2-11 | IGLJ1 | 2952 | ARSYGGSYST | 4142 | CSYAGTY | Anna Wec et al., 2020 |
| | VGGSNYVSWYQQHPGKAPKLLVYDV | (Human) | (Human) | (Human) | (Human) | | VGY | | | (https://science.science |
| | TKRPSGVPDRFSGSKSGNTASLTISGL | | | | | | | | | mag.org/content/early/ |
| | QAEDEADYYCCSYAGTYIFGTGTKLTV | | | | | | | | | 2020/06/15/science.abc |
| | L | | | | | | | | | 7424) |
| mAb-165 | QSVLTQPPSVSGSPGQSVSISCSGTSS | IGHV3-15 | IGHJ4 | IGLV2-18 | IGLJ1 | 2953 | SWNDVGWA | 4143 | CSYRSDNT | Anna Wec et al., 2020 |
| | DFGNYNRISWYQQTPGTAPKVIIYEVN | (Human) | (Human) | (Human) | (Human) | | FTF | | YI | (https://science.science |
| | SRPSGVPDRFSGSKSGNTASLTITGLQ | | | | | | | | | mag.org/content/early/ |
| | AEDEADYYCCSYRSDNTYIFGGGTKVT | | | | | | | | | 2020/06/15/science.abc |
| | VL | | | | | | | | | 7424) |
| mAb-166 | ETTLTQSPLFLPVTLGQPASISCRSSQRL | IGHV1-69 | IGHJ6 | IGKV2-30 | IGKJ4 | 2954 | AAEERSGTNH | 4144 | MQGTEW | Anna Wec et al., 2020 |
| | VHTNGNTYLNWFQQRPGHSPRRLIY | (Human) | (Human) | (Human) | (Human) | | NYYGLDV | | PRT | (https://science.science |
| | QVSNRDSGVPDRFSGSGSGTDFTLKIS | | | | | | | | | mag.org/content/early/ |
| | RVEAEDVGVYYCMQGTEWPRTFGG | | | | | | | | | 2020/06/15/science.abc |
| | GTKLEIK | | | | | | | | | 7424) |
| mAb-167 | QPVLTQPPSASGTPGQRVTISCSGGSS | IGHV3-53 | IGHJ3 | IGLV1-44 | IGLJ1 | 2955 | ARDREMAIIT | 4145 | AAWDDSL | Anna Wec et al., 2020 |
| | NIGSNSVNWYQQLPGTAPKLLIYSNSQ | (Human) | (Human) | (Human) | (Human) | | ERSYGLDV | | NTFRYV | (https://science.science |
| | RPSGVPDRFSGSKSGTSASLAISGLQSE | | | | | | | | | mag.org/content/early/ |
| | DEADYYCAAWDDSLNTFRYVFGTGTK | | | | | | | | | 2020/06/15/science.abc |
| | VTVL | | | | | | | | | 7424) |
| mAb-168 | ETTLTQSPSSLSASVGDRVNITCRASQS | IGHV3-23 | IGHJ3 | IGKV1-39 | IGKJ3 | 2956 | AKDRYCSGGS | 4146 | QQSYNTFF | Anna Wec et al., 2020 |
| | ISTYLTWYQQKPGKAPKLLIYGASSLHS | (Human) | (Human) | (Human) | (Human) | | CFYDAFDI | | T | (https://science.science |
| | GVPSRFTGVGSGTEFTLSISSLQPEDFA | | | | | | | | | mag.org/content/early/ |
| | TYYCQQSYNTFFTFGGGTKVDIK | | | | | | | | | 2020/06/15/science.abc |
| | | | | | | | | | | 7424) |
| mAb-169 | DIRLTQSPSSLSASVGDRVTITCRASQA | IGHV3-30 | IGHJ4 | IGKV1D- | IGKJ5 | 2957 | AREAQSSGRA | 4147 | QHYDSYPT | Anna Wec et al., 2020 |
| | IAGWLAWYQQKPGRAPKSLIYRASSL | (Human) | (Human) | 16 | (Human) | | GCLDA | | A | (https://science.science |
| | QSGVPSRFSGSGSGTDFSLTISNLQPE | | | (Human) | | | | | | mag.org/content/early/ |
| | DSATYYCQHYDSYPTAFGQGTRLEIK | | | | | | | | | 2020/06/15/science.abc |
| | | | | | | | | | | 7424) |
| mAb-17 | ETGLTQIPFSLPVTFGQPASISCRFSQR | IGHV1-69 | IGHJ3 | IGKV2-30 | IGKJ1 | 2958 | ARDPSIHYTG | 4148 | MQGTEWL | 2020/06/15/science.abc |
| | LVHTDGNTNLNWFLQRPGQFPRGLIY | (Human) | (Human) | (Human) | (Human) | | NHHWYDLDI | | GT | Anna Wec et al., 2020 |
| | KVFNRDSGVPDRFRGSGSGIDFTLKIS | | | | | | | | | (https://science.science |
| | RVEVEDVGIYYGMQGTEWLGTFGQG | | | | | | | | | mag.org/content/early/ |
| | TKVEIK | | | | | | | | | 7424) |
| mAb-170 | ETTLTQSPLSLAVTLGQPASISCRSSLGL | IGHV1-69 | IGHJ6 | IGKV2-30 | IGKJ1 | 2959 | VRDSDPYTAT | 4149 | MQGTEW | Anna Wec et al., 2020 |
| | VHTNGNTYLNWFQQRPGQSPRRLIYR | (Human) | (Human) | (Human) | (Human) | | VRSNHYWYA | | PRT | (https://science.science |
| | VSNRDSGVPDRFSGSGSVTDFTLTISR | | | | | | MDV | | | mag.org/content/early/ |
| | VEAEDIGVYYCMQGTEWPRTFGQGT | | | | | | | | | 2020/06/15/science.abc |
| | KVEIK | | | | | | | | | 7424) |
| mAb-171 | ETTLTQSPLSLSVTLGQAASISCTCSQT | IGHV1-69 | IGHJ6 | IGKV2-30 | IGKJ3 | 2960 | ARDPTFLNSG | 4150 | MQTTDWP | Anna Wec et al., 2020 |
| | AVHSDGNTYLNWFHQRPGQSPRRLIY | (Human) | (Human) | (Human) | (Human) | | NHFWYAVDI | | RT | (https://science.science |
| | KVSNRDSGVPDRFSGSGSGTDFTLKIS | | | | | | | | | mag.org/content/early/ |
| | RVEAEDVGIYYCMQTTDWPRTFGQG | | | | | | | | | 2020/06/15/science.abc |
| | TKVDIK | | | | | | | | | 7424) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| mAb-172 | Ab | HKU1 | 229E, | | | S; Unk | B-cells; SARS-CoV1 | 855 | QVQLQESGRGLVKPSETLSLTCTVSGGSI | 1922 |
| | | (weak), | NL63, | | | | Human Patient | | TSSRYYWGWIRQPPGRNLEWIGSIHYS | |
| | | SARS-CoV1 | OC43 | | | | | | GTTSYNPSLWSRVAISVDTAQNQFSLRL | |
| | | (weak), | | | | | | | NSVTAADTAVYYCAAPAPSNHESWSGT | |
| | | SARS-CoV2 | | | | | | | DWFDPWGQGILVTVSS | |
| | | (weak) | | | | | | | | |
| mAb-173 | Ab | HKU1 | 229E, | | | S; Unk | B-cells; SARS-CoV1 | 856 | QVQLVQSGAEVKGPGASVKVSCKVSGY | 1923 |
| | | (weak), | NL63, | | | | Human Patient | | SFITYDITWVRQAPGQGLEWMGWISTK | |
| | | SARS-CoV1 | OC43 | | | | | | SGDTRYAQNVQGRVTMTTDTSTNTAY | |
| | | (weak), | | | | | | | MELRNLKSDDTALYYCARTTPRGWEQ | |
| | | SARS-CoV2 | | | | | | | WPVLEYWGQGTLVTVSS | |
| | | (weak) | | | | | | | | |
| mAb-174 | Ab | HKU1 | 229E, | | | S; Unk | B-cells; SARS-CoV1 | 857 | EVQLVESGGGLVQPGGSLRLSCEASGEN | 1924 |
| | | (weak), | NL63, | | | | Human Patient | | FNSYSMSWVRQAPGKGLEWLSYISSRSS | |
| | | SARS-CoV1 | OC43 | | | | | | TIKYASSVQGRFTVSRDNAKKSVYLQMN | |
| | | (weak), | | | | | | | SLRDEDTAVYFCARELDSETYYNYNSLDV | |
| | | SARS-CoV2 | | | | | | | WGQGTTVTVSS | |
| | | (weak) | | | | | | | | |
| mAb-175 | Ab | HKU1 | 229E, | | | S; Unk | B-cells; SARS-CoV1 | 858 | QVQLVESGGEVKKPGASVKVSCKASGY | 1925 |
| | | (weak), | NL63, | | | | Human Patient | | TLSSYPISWVRQAPGHGLEWMGWINT | |
| | | SARS-CoV1 | OC43 | | | | | | YNGRTNYEQMLQGRVTMTTDTSTSTAY | |
| | | (weak), | | | | | | | MELRSLRSDDTAVYYCARVVFRHGQYD | |
| | | SARS-CoV2 | | | | | | | DSSGRLAFDIWGQGTMVTVSS | |
| | | (weak) | | | | | | | | |
| mAb-176 | Ab | HKU1 | 229E, | | | S; Unk | B-cells; SARS-CoV1 | 859 | QVQLVQSGAELKKPGSSVRVSCKAAGG | 1926 |
| | | (weak), | NL63, | | | | Human Patient | | TLTNYAISWVRQAPGQGFEWMGGIMP | |
| | | SARS-CoV1 | OC43 | | | | | | VSHTAGYAQKFQGRVTFTADESATTAY | |
| | | (weak), | | | | | | | MDLTSLRPEDTAIYYCARDPSIHYTGNH | |
| | | SARS-CoV2 | | | | | | | HWYDLDIWGQGTMVTVSS | |
| | | (weak) | | | | | | | | |
| mAb-177 | Ab | HKU1 | 229E, | | | S; Unk | B-cells; SARS-CoV1 | 860 | EVQLLESGAEVKKPGSSVKVSCKTSGGT | 1927 |
| | | (weak), | NL63, | | | | Human Patient | | FSTHAISWVRQAPGQGPEWMGGIIPLF | |
| | | SARS-CoV1 | OC43 | | | | | | GTSEHAQRFQARVKFTADESTSTAYMEL | |
| | | (weak), | | | | | | | SSLTPEDTAVYYCVRDSDPYTATSRNNH | |
| | | SARS-CoV2 | | | | | | | YWYGMDVWGQGTTVTVSS | |
| | | (weak) | | | | | | | | |
| mAb-178 | Ab | SARS-CoV1 | 229E, | | | S; Unk | B-cells; SARS-CoV1 | 861 | EVQLLESGGGVVQPGTSLRLSCAVSGF | 1928 |
| | | (weak), | HKU1, | | | | Human Patient | | MFKNYAIHWVRQAPGKGLEWVAVISF | |
| | | SARS-CoV2 | NL63, | | | | | | DGSDISYTESVQGRFTISRDNSENMLYL | |
| | | (weak) | OC43 | | | | | | QMNSLRAEDTAMYYCAREPDGIGAAGI | |
| | | | | | | | | | SGYWGQGTLVTVSS | |
| mAb-179 | Ab | SARS-CoV1 | 229E, | | | S; Unk | B-cells; SARS-CoV1 | 862 | EVQLVESGGGLVKPGGSLRLSCVASGFT | 1929 |
| | | (weak), | HKU1, | | | | Human Patient | | FSDFYMSWIRQAPGKGLEWVSYISGSG | |
| | | SARS-CoV2 | NL63, | | | | | | DTIYYADSVKGRFTVSRDNAKNSLFLQM | |
| | | (weak) | OC43 | | | | | | SSLGAEDTAMYYCAREMATSFGYYFVLD | |
| | | | | | | | | | VWGQGTTVTVSS | |
| mAb-18 | Ab | SARS-CoV1, | 229E, | | | S; Unk | B-cells; SARS-CoV1 | 863 | QVQLQQWGAGLLKPSETLSLSCAVYGG | 1930 |
| | | SARS-CoV2 | HKU1, | | | | Human Patient | | SFSGFYWSWIRQPPGKGLEWIGEINHS | |
| | | | NL63, | | | | | | GSANYNPSLMSRVTISMDTSKKQFSLQL | |
| | | | OC43 | | | | | | RSVTAADTAVYYCARGQESPIVGVTGR | |
| | | | | | | | | | WFDPWGQGTLVTVSS | |
| mAb-180 | Ab | SARS-CoV1 | 229E, | | | S; Unk | B-cells; SARS-CoV1 | 864 | EVQLVESGGGVVQPGRSLRLSCAASEFT | 1931 |
| | | (weak), | HKU1, | | | | Human Patient | | FSTYAMHWVRQAPGKGLEWVALISYD | |
| | | SARS-CoV2 | NL63, | | | | | | GINKYYADSVKGRFAISRDNSKNTLYLQV | |
| | | (weak) | OC43 | | | | | | NSLRADDTAVYYCVRPYSGSYTNWFDL | |
| | | | | | | | | | WGQGTLVTVSS | |
| mAb-181 | Ab | SARS-CoV1 | 229E, | | | S; Unk | B-cells; SARS-CoV1 | 865 | QVQLVQSGAEVKKPGSSVKVSCKASGG | 1932 |
| | | (weak), | HKU1, | | | | Human Patient | | MLSDYAISWVRQAPGQRLEWMGGIM | |
| | | SARS-CoV2 | NL63, | | | | | | PAFGSPGYAQIFRGRATISADVSTSTAYL | |
| | | (weak) | OC43 | | | | | | ELTSLKPEDTAVYYCARDPSILNTGNHH | |
| | | | | | | | | | WYDLDIWGQGTMVTVSS | |
| mAb-182 | Ab | 229 | NL63, | | | S; Unk | B-cells; SARS-CoV1 | 866 | QVQLVQSGAEVKMPGSSVKVSCKVSG | 1933 |
| | | (weak), | OC43, | | | | Human Patient | | GRFSDYAISWLRQAPLEGLEWMGGIVP | |
| | | HKU1 | SARS- | | | | | | HLNRKGYSQKFQDRLTFTADDSTSTAYM | |
| | | (weak), | CoV1 | | | | | | ELSGLTSEDTAVYYCARDPTFLNTGNHF | |
| | | SARS-CoV2 | | | | | | | WYAVDIWGQGTTVTVSS | |
| | | (weak) | | | | | | | | |
| mAb-183 | Ab | HKU1, SARS- | 229E, | | | S; Unk | B-cells; SARS-CoV1 | 867 | QVQLVQSGAEVKKPGSSVKVSCKASGG | 1934 |
| | | CoV2 (weak) | NL63, | | | | Human Patient | | TFSTHAISWVRQAPGHGPEWLGGILPLF | |
| | | | OC43, | | | | | | GTSESAQRFQARVKITADESTSTAYMELS | |
| | | | SARS- | | | | | | SLTSEDTAVYYCVRDSDPYTATSRNNHY | |
| | | | Cov1 | | | | | | WYAMDVWGQGTTVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| mAb-172 | EIVMTQSPASLSASVGDRVTITCRAGQ | IGHV4-39 | IGHJ5 | IGKV1-39 | IGKJ3 | 2961 | AAPAPSNHES | 4151 | QQSYSTPP | Anna Wec et al., 2020 |
| | SISTNLCWYQQRQGKAPKLLIYAASSL | (Human) | (Human) | (Human) | (Human) | | WSGTDWFDP | | T | (https://science.science |
| | RSGVPSRFSGSGSGTDFTLTISSLQPED | | | | | | | | | mag.org/content/early/ |
| | FATYYCQQSYSTPPTFGGGTKVDIK | | | | | | | | | 2020/06/15/science.abc |
| | | | | | | | | | | 7424) |
| mAb-173 | DIQVTQSPATLSVSPGERVTLSCRASQ | IGHV1-18 | IGHJ4 | IGKV3-15 | IGKJ4 | 2962 | ARTTPRGWE | 4152 | HQYNKWP | Anna Wec et al., 2020 |
| | SISNTLAWYQQKPGQAPRLLIYGASTR | (Human) | (Human) | (Human) | (Human) | | QWPVLEY | | PIT | (https://science.science |
| | ATGIPARFSGSGSGTEFTLTISSLQSEDF | | | | | | | | | mag.org/content/early/ |
| | AVYYCHQYNKWPPITFGGGTKVEIK | | | | | | | | | 2020/06/15/science.abc |
| | | | | | | | | | | 7424) |
| mAb-174 | DIQMTQSPSSLSASVGDRVTISCRASQ | IGHV3-48 | IGHJ6 | IGKV1-16 | IGKJ2 | 2963 | ARELDSETYY | 4153 | KQYNSYPY | Anna Wec et al., 2020 |
| | GISTFLAWFQQRPGKAPKSLIYAASKL | (Human) | (Human) | (Human) | (Human) | | NYNSLDV | | T | (https://science.science |
| | QSGVPSRFSGSDSGPDFTLTIDNLRPE | | | | | | | | | mag.org/content/early/ |
| | DSATYYCKQYNSYPYTFGQGTKLEIK | | | | | | | | | 2020/06/15/science.abc |
| | | | | | | | | | | 7424) |
| mAb-175 | EIVMTQSPSSLSASVGDSVTITCRASQS | IGHV1-18 | IGHJ3 | IGKV1-39 | IGKJ1 | 2964 | ARVVFRHGQ | 4154 | QQSYSDS | Anna Wec et al., 2020 |
| | ISNYLNWYQDKPGKAPELLIYAASNLQ | (Human) | (Human) | (Human) | (Human) | | YDDSSGRLAF | | WT | (https://science.science |
| | SGVPSRFSGSGSGTDFTLTISSLQPEDF | | | | | | DI | | | mag.org/content/early/ |
| | ATYYCQQSYSDSWTFGQGTKVDIK | | | | | | | | | 2020/06/15/science.abc |
| | | | | | | | | | | 7424) |
| mAb-176 | ETTLTQSPLSLPVTLGQPASISCRSSQRI | IGHV1-69 | IGHJ3 | IGKV2-30 | IGKJ1 | 2965 | ARDPSIHYTG | 4155 | MQGTEW | Anna Wec et al., 2020 |
| | VHTDGNTYLNWFLQRPGQSPRRLIYK | (Human) | (Human) | (Human) | (Human) | | NHHWYDLDI | | PRT | (https://science.science |
| | VSNRDSGVPDRFSGSGSGTDFTLKISR | | | | | | | | | mag.org/content/early/ |
| | VEAEDVGIYYCMQGTEWPRTFGQGT | | | | | | | | | 2020/06/15/science.abc |
| | KVEIK | | | | | | | | | 7424) |
| mAb-177 | DIQMTQSPLSLPVTLGQPASISCRSSQS | IGHV1-69 | IGHJ6 | IGKV2-30 | IGKJ3 | 2966 | VRDSDPYTAT | 4156 | MQGTDW | Anna Wec et al., 2020 |
| | VVHSDGNTYLNWFHQRPGQSPRRLIY | (Human) | (Human) | (Human) | (Human) | | SRNNHYWYG | | PRT | (https://science.science |
| | KVSNRDSGVPDRFSGSGSGTDFTLKIS | | | | | | MDV | | | mag.org/content/early/ |
| | RVEAEDIGVYYCMQGTDWPRTFGQG | | | | | | | | | 2020/06/15/science.abc |
| | TKVDIK | | | | | | | | | 7424) |
| mAb-178 | GIVLTQSPDSLAVSVGERATINCKSSQT | IGHV3-30 | IGHJ4 | IGKV4-1 | IGKJ3 | 2967 | AREPDGIGAA | 4157 | QQYYTTPY | Anna Wec et al., 2020 |
| | VLYSSKNKHYLAWYQQKPGQPPKLLT | (Human) | (Human) | (Human) | (Human) | | GISGY | | T | (https://science.science |
| | SSPSTREPGVPDRFSGSGSGTDFTLTIS | | | | | | | | | mag.org/content/early/ |
| | SLQAEDVAVYYCQQYYTTPYTFGQGT | | | | | | | | | 2020/06/15/science.abc |
| | KVDIK | | | | | | | | | 7424) |
| mAb-179 | EIVMTQTPSSLSASVGDRVTITCRASQ | IGHV3-11 | IGHJ6 | IGKV1-39 | IGKJ3 | 2968 | AREMATSFGY | 4158 | QQTYSTPP | Anna Wec et al., 2020 |
| | SISNYVNWYQQKPGRAPNLLIYAASSL | (Human) | (Human) | (Human) | (Human) | | YFVLDV | | EGPT | (https://science.science |
| | QSGVSSRFSGSGSGTDFTLTISSLQPED | | | | | | | | | mag.org/content/early/ |
| | FATYYCQQTYSTPPEGPTFGPGTKVEIK | | | | | | | | | 2020/06/15/science.abc |
| | | | | | | | | | | 7424) |
| mAb-18 | QPVLTQPASVSGSPGQSVTISCTGTSS | IGHV4-34 | IGHJ5 | IGLV2-23 | IGLJ3 | 2969 | ARGQESPIVG | 4159 | CSYAGASP | Anna Wec et al., 2020 |
| | DVGSYSDVGNYVSWYQHHPGKAPKL | (Human) | (Human) | (Human) | (Human) | | VTGRWFDP | | FVV | (https://science.science |
| | MIYEVRTRPSWVSTRFSGSKSGTTASL | | | | | | | | | mag.org/content/early/ |
| | TISGLQAEDEADYYCCSYAGASPFVVF | | | | | | | | | 2020/06/15/science.abc |
| | GGGTKLTVL | | | | | | | | | 7424) |
| mAb-180 | EIVMTQSPGTLSSSPGERATLSCRASQ | IGHV3-30 | IGHJ4 | IGKV3-20 | IGKJ2 | 2970 | VRPYSGSYTN | 4160 | QQYGSSYT | Anna Wec et al., 2020 |
| | SVSSRYLAWYQQKPGQAPRLLIYGTSN | (Human) | (Human) | (Human) | (Human) | | WFDL | | | (https://science.science |
| | RATGIPDRFSGSGSGTDFTLTISRLEPE | | | | | | | | | mag.org/content/early/ |
| | DFAVYYCQQYGSSYTFGPGTKLEIK | | | | | | | | | 2020/06/15/science.abc |
| | | | | | | | | | | 7424) |
| mAb-181 | DIVMTQSPLSLPVGLGQSASISCRSSQ | IGHV1-69 | IGHJ3 | IGKV2-30 | IGKJ1 | 2971 | ARDPSILNTG | 4161 | MQATEWP | Anna Wec et al., 2020 |
| | RVVHTDGNTYLNWFHQRPGQSPRRLI | (Human) | (Human) | (Human) | (Human) | | NHHWYDLDI | | RT | (https://science.science |
| | YKVSNRDSGVPDRFSGSGSGTDFTLRI | | | | | | | | | mag.org/content/early/ |
| | SRVEAEDVGVYYCMQATEWPRTFGQ | | | | | | | | | 2020/06/15/science.abc |
| | GTKVEIK | | | | | | | | | 7424) |
| mAb-182 | DIRLTQSPLSLSVTLGQAASISCTCSQSA | IGHV1-69 | IGHJ6 | IGKV2-30 | IGKJ1 | 2972 | ARDPTFLNTG | 4162 | MQTTDWP | Anna Wec et al., 2020 |
| | VHSDGTTYFNWFHQRPGQSPRRLIYK | (Human) | (Human) | (Human) | (Human) | | NHFWYAVDI | | RT | (https://science.science |
| | VSNRDSGVPDRFIGSGSGTHFTLKISR | | | | | | | | | mag.org/content/early/ |
| | VEAEDVGVYYCMQTTDWPRTFGQGT | | | | | | | | | 2020/06/15/science.abc |
| | KVEIK | | | | | | | | | 7424) |
| mAb-183 | ETTLTQSPLSLPVTLGQPASISCRSSQS | IGHV1-69 | IGHJ6 | IGKV2-30 | IGKJ1 | 2973 | VRDSDPYTAT | 4163 | MQGTDW | Anna Wec et al., 2020 |
| | VVHSDGNTYLNWFHQRPGQSPRRLIY | (Human) | (Human) | (Human) | (Human) | | SRNNHYWYA | | PRT | (https://science.science |
| | KVSNRDSGVPDRFSGSGSGTDFTLKIS | | | | | | MDV | | | mag.org/content/early/ |
| | RVEAEDIGVYYCMQGTDWPRTFGQG | | | | | | | | | 2020/06/15/science.abc |
| | TKVEIK | | | | | | | | | 7424) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| mAb-184 | Ab | HKU1 | 229E, | | | S; Unk | B-cells; SARS-CoV1 | 868 | QVQLVQSGAEVKKPGSSVKVSCKVSGG | 1935 |
| | | (weak), | NL63, | | | | Human Patient | | TFSNYAISWLRQAPGQGPEWMGGIIPA | |
| | | SARS-CoV2 | OC43, | | | | | | LSRVGYVRKFQARLTISADELTTTAYMDL | |
| | | (weak) | SARS- | | | | | | SSLTSEDTAVYYCARDPSFLNTGNHFWY | |
| | | | CoV1 | | | | | | DFDLWGQGTTVTVSS | |
| mAb-185 | Ab | SARS-CoV2 | 229E, | | | S; Unk | B-cells; SARS-CoV1 | 869 | QVQLQQWGAGLLKPSETLSLTCAVYGG | 1936 |
| | | (weak) | HKU1, | | | | Human Patient | | SFSGFYWTWIRQPPGKGLEWIGEINHS | |
| | | | NL63, | | | | | | GSSNYDLSLKSRVTMSVDTSKNQFSLKLS | |
| | | | OC43, | | | | | | SVTAADTAVYYCARGMISPRIPRTTRQR | |
| | | | SARS- | | | | | | WFDTWGQGTLVTVSS | |
| | | | CoV1 | | | | | | | |
| mAb-186 | Ab | SARS-CoV2 | 229E, | | | S; Unk | B-cells; SARS-CoV1 | 870 | QVQLQQWGAGLLKPSETLSLTCGVYGG | 1937 |
| | | (weak) | HKU1, | | | | Human Patient | | SFSGYFWSWIRQSPGKGLEWIGEINHSR | |
| | | | NL63, | | | | | | SMSYNPSLKSRITMSVDTSKNQFSLNLN | |
| | | | OC43, | | | | | | SVTAADTAVYFCARGKAHRNDFWSGYY | |
| | | | SARS- | | | | | | PHWFDPWGQGILVTVSS | |
| | | | CoV1 | | | | | | | |
| mAb-187 | Ab | SARS-CoV2 | 229E, | | | S; Unk | B-cells; SARS-CoV1 | 871 | QVQLVQSGAEVKKPGSSVKISCKISGDTF | 1938 |
| | | (weak) | HKU1, | | | | Human Patient | | STNAISWLRQAPGREPEWMGGIVPLVG | |
| | | | NL63, | | | | | | PASYAQRPQGRLTITADEFTNTAYLELNS | |
| | | | OC43, | | | | | | LRSEDTATYYCARDSDPYTATRRHNHYW | |
| | | | SARS- | | | | | | YAMDVWGQGTTVTVSS | |
| | | | CoV1 | | | | | | | |
| mAb-188 | Ab | SARS-CoV2 | 229E, | | | S; Unk | B-cells; SARS-CoV1 | 872 | EVQLLESGPGLVKPSETLSLTCTVSGGSI | 1939 |
| | | (weak) | HKU1, | | | | Human Patient | | NSQYWNWIRQSPGKGLEWIGYVYYSGS | |
| | | | NL63, | | | | | | TNYNPSLKSRVTMSVDTSKNHFSLNLRS | |
| | | | OC43, | | | | | | VTAADTAVYYCARGLVVRYFDGFPSGPII | |
| | | | SARS- | | | | | | GAFDIWNQGTTVTVSS | |
| | | | CoV1 | | | | | | | |
| mAb-189 | Ab | SARS-CoV2 | 229E, | | | S; Unk | B-cells; SARS-CoV1 | 873 | QVQLVQSGAEVKKPGSSVKVSCKVSGG | 1940 |
| | | (weak) | HKU1, | | | | Human Patient | | TFSSHAISWVRQAPGQRPEWMGGIMP | |
| | | | NL63, | | | | | | IFGESKDTQKFQGRVTFTVDESTNTAYM | |
| | | | OC43, | | | | | | ELTSLKSEDTAIYYCVRDPDPYTATVRHN | |
| | | | SARS- | | | | | | HYWHGMDVWGQGTTVTVSS | |
| | | | CoV1 | | | | | | | |
| mAb-19 | Ab | OC43, SARS- | 229E, | | | S; Unk | B-cells; SARS-CoV1 | 874 | QVQLQQWGAGLLKPSETLSLTCAVYGG | 1941 |
| | | CoV1, SARS- | HKU1, | | | | Human Patient | | SFSGFHWSWIRQPPGKGLEWIGEVNHS | |
| | | CoV2 | NL63 | | | | | | GSTKYNPSLKSRVTVSVDTSKNQFSLRLS | |
| | | | | | | | | | SVTAADTAIYYCARGSLSREYDFLTAPQN | |
| | | | | | | | | | GPWFDSWGQGALVTVSS | |
| mAb-190 | Ab | SARS-CoV2 | 229E, | | | S; Unk | B-cells; SARS-CoV1 | 875 | EVQLLESGPGLVKPSETLSLTCTVSGGSIS | 1942 |
| | | (weak) | HKU1, | | | | Human Patient | | SNNLYWGWIRQPPGKGLEWIGSIYYSG | |
| | | | NL63, | | | | | | STYYNPSLKSRVIIPVDTSKDQFSLRLSSV | |
| | | | OC43, | | | | | | TAADTAVYYCARHSQKDIVLIPAAQSPIF | |
| | | | SARS- | | | | | | DYWGQGTLVTVSS | |
| | | | CoV1 | | | | | | | |
| mAb-191 | Ab | SARS-CoV2 | 229E, | | | S; Unk | B-cells; SARS-CoV1 | 876 | EVQLVESGGGLVQPGGSLRLSCSASGFT | 1943 |
| | | (weak) | HKU1, | | | | Human Patient | | FSRYAMHWVRQAPGKGLEYVSAINLNG | |
| | | | NL63, | | | | | | DSTYYTDSVRGRFTISRDNSKNTLFLQMS | |
| | | | OC43, | | | | | | NVRPEDTAFYYCVKDGGYYDSSGPGHW | |
| | | | SARS- | | | | | | GQGTLVTVSS | |
| | | | CoV1 | | | | | | | |
| mAb-192 | Ab | SARS-CoV2 | 229E, | | | S; Unk | B-cells; SARS-CoV1 | 877 | QVQLVQSGGGVVQPGGSLRLSCADSG | 1944 |
| | | (weak) | HKU1, | | | | Human Patient | | NAFIANPMHWVRQAPGKGLEWLALIST | |
| | | | NL63, | | | | | | DGNNRHYADSVKGRFTFSRDNSKNSLYL | |
| | | | OC43, | | | | | | QMDSLRPEDTGVYYCARESRSSGRAGC | |
| | | | SARS- | | | | | | FDSWGQGTLVTVSS | |
| mAb-193 | Ab | SARS-CoV2 | 229E, | | | S; Unk | B-cells; SARS-CoV1 | 878 | QVQLVESGGGVVQPGTSLRLSCAATGF | 1945 |
| | | (weak) | HKU1, | | | | Human Patient | | TFTTYPMHWVRQAPGKGLEWVAVISY | |
| | | | NL63, | | | | | | DGMNQYYADFLKGRFTISRDNSKNTLYL | |
| | | | OC43, | | | | | | QMNSLRADDTAVYYCARAYGGNYQNH | |
| | | | SARS- | | | | | | FDHWGQGTLVTVSS | |
| | | | CoV1 | | | | | | | |
| mAb-194 | Ab | SARS-CoV2 | 229E, | | | S; Unk | B-cells; SARS-CoV1 | 879 | EVQLVQWGAGLLKPSETLSLRCAVYGGS | 1946 |
| | | (weak) | HKU1, | | | | Human Patient | | FNGFYWTWIRQAPGQGLEWIAEINHSG | |
| | | | NL63, | | | | | | TTNYNPSLKSRVTISIDTSKKQFSLSLKSVT | |
| | | | OC43, | | | | | | AADTAMYFCARGTISPIVGVPTPVVPRR | |
| | | | SARS- | | | | | | GRSWFDPWGQGTLVTVSS | |
| | | | CoV1 | | | | | | | |
| mAb-195 | Ab | SARS-CoV2 | 229E, | | | S; Unk | B-cells; SARS-CoV1 | 880 | QVQLVQSGAEVKKPGSSVKVSCKASGG | 1947 |
| | | (weak) | HKU1, | | | | Human Patient | | TFSTHAISWVRQAPGHGPEWLGGIIPLF | |
| | | | NL63, | | | | | | GTSESAQRFQARVKITADESTSTAYMELS | |
| | | | OC43, | | | | | | SLTSEDTAVYYCVRDSDPYTATSRNNHY | |
| | | | SARS- | | | | | | WYAMDVWGQGTTVTVSS | |
| | | | CoV1 | | | | | | | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| mAb-184 | ETTLTQSPLSLPVTLGQPASISCTSSQTV | IGHV1-69 | IGHJ4 | IGKV2-30 | IGKJ2 | 2974 | ARDPSFLNTG | 4164 | MQGTEW | Anna Wec et al., 2020 |
| | VHTDRNTYLNWYHQRPGQSPRRLIYK | (Human) | (Human) | (Human) | (Human) | | NHFWYDFDL | | PRT | (https://science.science |
| | VSNRDSGVPDRFSGSGSGSHFTLKISR | | | | | | | | | mag.org/content/early/ |
| | VEAEDVGVYYCMQGTEWPRTFGQGT | | | | | | | | | 2020/06/15/science.abc |
| | KLEIK | | | | | | | | | 7424) |
| mAb-185 | QPGLTQPASVSGSPGQSITISCTGTSG | IGHV4-34 | IGHJ4 | IGLV2-23 | IGLJ3 | 2975 | ARGMISPRIP | 4165 | CSYAGDDT | Anna Wec et al., 2020 |
| | DVGSDNLVSWYQRHPGKAPKLMIYE | (Human) | (Human) | (Human) | (Human) | | RTTRQRWFD | | VV | (https://science.science |
| | GSKRPSGVSHRFSGSNSGNTASLTISG | | | | | | T | | | mag.org/content/early/ |
| | LQAEDDADYYCCSYAGDDTVVFGGGT | | | | | | | | | 2020/06/15/science.abc |
| | KLTVL | | | | | | | | | 7424) |
| mAb-186 | DIVLTQSPGTLSLSPGERVTLSCRASQSI | IGHV4-34 | IGHJ5 | IGKV3-20 | IGKJ1 | 2976 | ARGKAHRND | 4166 | HQYGGSPT | Anna Wec et al., 2020 |
| | SSNYLAWYQQKPGQAPRLLISDASSRA | (Human) | (Human) | (Human) | (Human) | | FWSGYYPHW | | T | (https://science.science |
| | TGIPDRFSGSGSGADFTLIISRLEPEDFA | | | | | | FDP | | | mag.org/content/early/ |
| | VYFCHQYGGSPTTFGQGTKVEIK | | | | | | | | | 2020/06/15/science.abc |
| | | | | | | | | | | 7424) |
| mAb-187 | ETTLTQSPLSLPVTLGQPASISCRSSQIA | IGHV1-69 | IGHJ6 | IGKV2-30 | IGKJ1 | 2977 | ARDSDPYTAT | 4167 | MQGTDW | Anna Wec et al., 2020 |
| | MHSDGNTYLSWFHQRPGQPPRRLIYK | (Human) | (Human) | (Human) | (Human) | | RRHNHYWYA | | PRT | (https://science.science |
| | ISNRDSGVPDRFSGSGSGTDFTLKISRV | | | | | | MDV | | | mag.org/content/early/ |
| | EAEDIGTFYCMQGTDWPRTFGQGTK | | | | | | | | | 2020/06/15/science.abc |
| | VEIK | | | | | | | | | 7424) |
| mAb-188 | ETTLTQSPATLSLSPGERATLSCRASQS | IGHV4-59 | IGHJ3 | IGKV3-11 | IGKJ4 | 2978 | ARGLVVRYFD | 4168 | QQRTSTLT | Anna Wec et al., 2020 |
| | VSSYLAWYQQKPGQAPRLLIYDAFNR | (Human) | (Human) | (Human) | (Human) | | GFPSGPIIGAF | | | (https://science.science |
| | ATGVPARFSGSGSGTDFTLTISSLEPED | | | | | | DI | | | mag.org/content/early/ |
| | FAVYYCQQRTSTLTFGGGTKVDIK | | | | | | | | | 2020/06/15/science.abc |
| | | | | | | | | | | 7424) |
| mAb-189 | ETTLTQSPLSLPVTLGQAASISCRSSQA | IGHV1-69 | IGHJ6 | IGKV2-30 | IGKJ1 | 2979 | VRDPDPYTAT | 4169 | MQGTEW | Anna Wec et al., 2020 |
| | VVHSDGNTYLNWFQQRPGQPPRRLIY | (Human) | (Human) | (Human) | (Human) | | VRHNHYWH | | PRT | (https://science.science |
| | KVSNRDSGVPDRFSGSGSGTVFTLKIS | | | | | | GMDV | | | mag.org/content/early/ |
| | RVEAEDIGVYYCMQGTEWPRTFGQG | | | | | | | | | 2020/06/15/science.abc |
| | TKVEIK | | | | | | | | | 7424) |
| mAb-19 | QSVLTQPPSVSAAPGQKVTISCSGSSS | IGHV4-34 | IGHJ5 | IGLV1-51 | IGLJ3 | 2980 | ARGSLSREYD | 4170 | ETWDSSLS | Anna Wec et al., 2020 |
| | NLGNNYVSWYQQLPGTAPKLLIYDNH | (Human) | (Human) | (Human) | (Human) | | FLTAPQNGP | | VVV | (https://science.science |
| | KRPSGIPDRFSGSKSGTSATLGITGLQT | | | | | | WFDS | | | mag.org/content/early/ |
| | GDEADYYCETWDSSLSVVVFGGGTKV | | | | | | | | | 2020/06/15/science.abc |
| | TVL | | | | | | | | | 7424) |
| mAb-190 | QSVLTQPPSVSATPGQKVTISCSGSSS | IGHV4-39 | IGHJ4 | IGLV1-51 | IGLJ3 | 2981 | ARHSQKDIVLI | 4171 | GTWDSRLS | Anna Wec et al., 2020 |
| | NIGNNYVSWYQQLPGTAPKLLIYDNN | (Human) | (Human) | (Human) | (Human) | | PAAQSPIFDY | | AVV | (https://science.science |
| | KRPSGIPDRFSGSKSGTSATLGITGLQT | | | | | | | | | mag.org/content/early/ |
| | GDEADYYCGTWDSRLSAVVFGGGTKL | | | | | | | | | 2020/06/15/science.abc |
| | TVL | | | | | | | | | 7424) |
| mAb-191 | DIRVTQSPATLSLSPGERATLSCRASEN | IGHV3-64D | IGHJ1 | IGKV3-11 | IGKJ3 | 2982 | VKDGGYYDSS | 4172 | QQRSNWP | Anna Wec et al., 2020 |
| | IAHYLAWYQQKPGQAPRLVIYDASSR | (Human) | (Human) | (Human) | (Human) | | GPGH | | QN | (https://science.science |
| | ATGIPGRFSGSGAGTDFTLTINSLEPED | | | | | | | | | mag.org/content/early/ |
| | FAVYYCQQRSNWPQNFGGGTKVDIK | | | | | | | | | 2020/06/15/science.abc |
| | | | | | | | | | | 7424) |
| mAb-192 | DIRLTQSPSSLSASVGDRVTITCRASQD | IGHV3-30 | IGHJ5 | IGKV1D-16 | IGKJ5 | 2983 | ARESRSSGRA | 4173 | QQYDSYPV | Anna Wec et al., 2020 |
| | ISSWLAWYQQKSGKAPKSLIYAASSLQ | (Human) | (Human) | (Human) | (Human) | | GCFDS | | T | (https://science.science |
| | NGVPSRFSGSRSGTDFTLTISSLQPEDL | | | | | | | | | mag.org/content/early/ |
| | GTYYCQQYDSYPVTFGQGTRLEIK | | | | | | | | | 2020/06/15/science.abc |
| | | | | | | | | | | 7424) |
| mAb-193 | DIVLTQTPDSLAVSLGERATINCKSSQS | IGHV3-30 | IGHJ4 | IGKV4-1 | IGKJ1 | 2984 | ARAYGGNYQ | 4174 | QQYYTMW | Anna Wec et al., 2020 |
| | VLYSSNNKNYLAWYQQKPGQPPKLLI | (Human) | (Human) | (Human) | (Human) | | NHFDH | | T | (https://science.science |
| | YWASTRESGVPDRFSGSGSGTDFTLTI | | | | | | | | | mag.org/content/early/ |
| | SSLQAEDVAVYYCQQYYTMWTFGQG | | | | | | | | | 2020/06/15/science.abc |
| | TKVEIK | | | | | | | | | 7424) |
| mAb-194 | QSVLTQPPSVSAAPGQKVTISCSGSSS | IGHV4-34 | IGHJ5 | IGLV1-51 | IGLJ1 | 2985 | ARGTISPIVGV | 4175 | GTWDSSLS | Anna Wec et al., 2020 |
| | NIGNDYVSWYQQLPGTAPKLLIYENYK | (Human) | (Human) | (Human) | (Human) | | PTPVVPRRGR | | VDNYV | (https://science.science |
| | RPSGIPDRFSGSKSGTSATLDITGLQTG | | | | | | SWFDP | | | mag.org/content/early/ |
| | DEADYYCGTWDSSLSVDNYVFGTGTK | | | | | | | | | 2020/06/15/science.abc |
| | LTVL | | | | | | | | | 7424) |
| mAb-195 | DIVLTQSPLSLPVTLGQPASISCRSSQS | IGHV1-69 | IGHJ6 | IGKV2-30 | IGKJ3 | 2986 | VRDSDPYTAT | 4176 | MQGTDW | Anna Wec et al., 2020 |
| | VVHSDGNTYLNWFHQRPGQSPRRLIY | (Human) | (Human) | (Human) | (Human) | | SRNNHYWYA | | PRT | (https://science.science |
| | KVSNRDSGVPDRFSGSGSGTDFTLKIS | | | | | | MDV | | | mag.org/content/early/ |
| | RVEAEDIGVYYCMQGTDWPRTFGQG | | | | | | | | | 2020/06/15/science.abc |
| | TKVDIK | | | | | | | | | 7424) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| mAb-196 | Ab | SARS-CoV2 | 229E, | | | S; Unk | B-cells; SARS-CoV1 | 881 | QVQLVQSGAEVREPGSSVKLSCKTSGGP | 1948 |
| | | (weak) | HKU1, | | | | Human Patient | | FSTHAFSWVRQAPGQRPEWMGGIMP | |
| | | | NL63, | | | | | | VFGESKDTQKFKGRVTFTADASTTTTYM | |
| | | | OC43, | | | | | | ELRSLKSDDTAIYYCVRDSDPYTATSSHN | |
| | | | SARS- | | | | | | HYWYAMDVWGQGTTVTVSS | |
| | | | CoV1 | | | | | | | |
| mAb-197 | Ab | SARS-CoV2 | 229E, | | | S; Unk | B-cells; SARS-CoV1 | 882 | QVQLVESGGGLVKPGGSLRLSCAASGFT | 1949 |
| | | (weak) | HKU1, | | | | Human Patient | | FSDYYMIWIRQAPGKGLEWLSYISHTAS | |
| | | | NL63, | | | | | | TIYYADSVKGRFTISRDNAKNSLFLQMNS | |
| | | | OC43, | | | | | | LTAEDTAVYYCARDRGSGVIDPWGQGT | |
| | | | SARS- | | | | | | LVTVSS | |
| | | | CoV1 | | | | | | | |
| mAb-2 | Ab | SARS-CoV1, | 229E, | | | S; Unk | B-cells; SARS-CoV1 | 883 | QVQLVESGGGLVQPGGSLRLSCSASGFT | 1950 |
| | | SARS-CoV2 | HKU1, | | | | Human Patient | | FSRFAMHWVRQAPGKGLEYVSAINLNG | |
| | | | NL63, | | | | | | DSTYYTDSVRGRFTISRDNSKNTLYLQMS | |
| | | | OC43 | | | | | | SVRPDDTAFYYCVKDGGYYDSSGPGHW | |
| | | | | | | | | | GQGTLVTVSS | |
| mAb-20 | Ab | OC43, SARS- | 229E, | | | S; Unk | B-cells; SARS-CoV1 | 884 | EVQLVESGPGLVKPSETLSLTCTVSGASV | 1951 |
| | | CoV1, SARS- | HKU1, | | | | Human Patient | | TAGSSYWGWIRQPPGKGLEWIGYMFSS | |
| | | CoV2 | NL63, | | | | | | GNTKYNPSLKSRVTISADTSKNQFSLRLS | |
| | | | OC43 | | | | | | SVTAADTAVYFCARVGWVRYFDWSKPY | |
| | | | | | | | | | YYFDLWGRGTLVTVSS | |
| mAb-21 | Ab | SARS-CoV1, | 229E, | | | S; Unk | B-cells; SARS-CoV1 | 885 | EVQLVESGGGLVQPGGSLRLSCAASGFT | 1952 |
| | | SARS-CoV2 | HKU1, | | | | Human Patient | | FSTSAMSWVRQAPGKGLEWVSRIGGG | |
| | | | NL63, | | | | | | GGRTKYADSVKGRFTISRDNSKNTLYLQ | |
| | | | OC43 | | | | | | MNSLRADDTAVYYCAKCDLVRYFDWLG | |
| | | | | | | | | | EENNWFDPWGQGTLVTVSS | |
| mAb-22 | Ab | SARS-CoV1, | 229E, | | | S; Unk | B-cells; SARS-CoV1 | 886 | QVQLVQSGAEVRKPGSSVKLSCKASGG | 1953 |
| | | SARS-CoV2 | HKU1, | | | | Human Patient | | TFSTHAISWVRQAPGQRPEWMGGIMP | |
| | | | NL63, | | | | | | IFGESKDTQKFQGRVTFTADESTTTAYM | |
| | | | OC43 | | | | | | ELRSLKSDDTAIYYCVRDSDPYTATVRNN | |
| | | | | | | | | | HYWYALDVWGPGTMVTVSS | |
| mAb-23 | Ab | SARS-CoV1, | 229E, | | | S; Unk | B-cells; SARS-CoV1 | 887 | EVQLVESGGGLSQPGGSLRLSCAASGFT | 1954 |
| | | SARS-CoV2 | HKU1, | | | | Human Patient | | VRTYCMNWVRQAPGKGLEWVSLVCG | |
| | | | NL63, | | | | | | DNIDYYPDSVKGRFSISRDDSKNTLLLH | |
| | | | OC43 | | | | | | MDSLRVEDTAVYYCARATPPGGGTGW | |
| | | | | | | | | | PYFDFWGQGTLVTVSS | |
| mAb-24 | Ab | SARS-CoV1 | 229E, | | | S; Unk | B-cells; SARS-CoV1 | 888 | QVQLVQSGAEMRKPGSSVKVSCKASGG | 1955 |
| | | (weak), | HKU1, | | | | Human Patient | | TFSRYCFSWVRQAPGQRLEWMGGIMS | |
| | | SARS-CoV2 | NL63, | | | | | | ILGAHYAQKFQGRVTFTADESTNTAYME | |
| | | | OC43 | | | | | | LISLTSEDTAVYYCAREEPSGTYHNYYGL | |
| | | | | | | | | | DVWGQGTTVTVSS | |
| mAb-25 | Ab | SARS-CoV1, | 229E, | | | S; Unk | B-cells; SARS-CoV1 | 889 | QVQLQESGPGLVKPSGTLSLTCAVSGGS | 1956 |
| | | SARS-CoV2 | HKU1, | | | | Human Patient | | ISSSDWCSWVRQPPGKGPEWIAEISHS | |
| | | | NL63, | | | | | | GSTNYNPSLKSRVTMSVDRSKNQFSLNL | |
| | | | OC43 | | | | | | NSVTAADTAVYYCAARIRGATHYDFWS | |
| | | | | | | | | | GFWAGPFDIWGQGTTVTVSS | |
| mAb-26 | Ab | SARS-CoV1, | 229E, | | | S; Unk | B-cells; SARS-CoV1 | 890 | EVQLQESGPGLVKPSGTLSLTCGVSGVSI | 1957 |
| | | SARS-CoV2 | HKU1, | | | | Human Patient | | SSSSWWSWVRQPPGRGLEWIGEISPSG | |
| | | | NL63, | | | | | | STSYNPSFRSRLTMSVDKSRNQLSLKLSS | |
| | | | OC43 | | | | | | VTAADTAVYYCARTQSNDFWSGYYTAA | |
| | | | | | | | | | FDLWGQGTMVTVSS | |
| mAb-27 | Ab | SARS-CoV1, | 229E, | | | S; Unk | B-cells; SARS-CoV1 | 891 | QVQLVQWGAGLLKPSETLSLTCAVYGG | 1958 |
| | | SARS-CoV2 | HKU1, | | | | Human Patient | | SFSGFHWSWIRQPPGKGLEWIGEINHS | |
| | | | NL63, | | | | | | GSTKYNPSLKSRVTISVDTSKNQFSLRLRS | |
| | | | OC43 | | | | | | VTAADTAIYYCARGSLSREYDFLTAPQN | |
| | | | | | | | | | GPWFDSWGQGALVTVSS | |
| mAb-28 | Ab | SARS-CoV1, | 229E, | | | S; Unk | B-cells; SARS-CoV1 | 892 | EVQLVESGPELKKPGSSVRVSCKASGGS | 1959 |
| | | SARS-CoV2 | HKU1, | | | | Human Patient | | FSNFAVSWVRQAPGQRLEWMGGVIPR | |
| | | | NL63, | | | | | | FGRSEYVQKFQGRVTITVDEAASTAYME | |
| | | | OC43 | | | | | | LSSLRSEDTAIYYCVLDTTSANPHNWYG | |
| | | | | | | | | | MDVWGQGTTVTVSS | |
| mAb-29 | Ab | SARS-CoV1, | 229E, | | | S; Unk | B-cells; SARS-CoV1 | 893 | QVQLVQSGGGVVNPGGSLRLSCVGSGF | 1960 |
| | | SARS-CoV2 | HKU1, | | | | Human Patient | | TFSDYYMGWIRQAPGKGLEVISYISSTGS | |
| | | | NL63, | | | | | | YIRDADSVKGRFTISRDNAENSVYLQMN | |
| | | | OC43 | | | | | | SLRGEDTAVYYCARMGPYGSGSFDYWG | |
| | | | | | | | | | LGTLVTVSS | |
| mAb-3 | Ab | SARS-CoV1, | 229E, | | | S; Unk | B-cells; SARS-CoV1 | 894 | QVQLQESGPGLVRPSETLSLTCTVSGDS | 1961 |
| | | SARS-CoV2 | HKU1, | | | | Human Patient | | VSSSDYHWGWIRQPPGKGLEWIGSIYY | |
| | | | NL63, | | | | | | GGRSHFNPSLKSRVAIFVDTSNNQFSLRL | |
| | | | OC43 | | | | | | NSVTASDTAVYFCAGRHQELLPMGSFD | |
| | | | | | | | | | MWAQGTTVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| mAb-196 | ETTLTQSPLSLPVTLGQPASISCRSSQV | IGHV1-69 | IGHJ6 | IGKV2-30 | IGKJ3 | 2987 | VRDSDPYTAT | 4177 | MQGTEW | Anna Wec et al., 2020 |
| | LVHSDGNTYLNWFQQRPGQSPRRLIY | (Human) | (Human) | (Human) | (Human) | | SSHNHYWYA | | PRT | (https://science.science |
| | KVSNRDSGVPDRFSGSGSGIDFTLKISR | | | | | | MDV | | | mag.org/content/early/ |
| | VEAEDIGVYYCMQGTEWPRTFGQGT | | | | | | | | | 2020/06/15/science.abc |
| | KVDIK | | | | | | | | | 7424) |
| mAb-197 | DIQLTQSPSSLSASVGDRVTITCRASQS | IGHV3-11 | IGHJ5 | IGKV1-39 | IGKJ4 | 2988 | ARDRGSGVID | 4178 | QQSYSTPL | Anna Wec et al., 2020 |
| | ISHYLNWYQQKPGKAPKLLIYAASNLQ | (Human) | (Human) | (Human) | (Human) | | P | | T | (https://science.science |
| | SGVPSRFSGNGSGTDFTLTISSLQPEDF | | | | | | | | | mag.org/content/early/ |
| | ATYFCQQSYSTPLTFGGGTKVDIK | | | | | | | | | 2020/06/15/science.abc |
| | | | | | | | | | | 7424) |
| mAb-2 | EIVLTQSPATLSLSPGERATLSCRASENI | IGHV3- | IGHJ1 | IGKV3-11 | IGKJ4 | 2989 | VKDGGYYDSS | 4179 | QQRSNWP | Anna Wec et al., 2020 |
| | AHYLAWYQQKPGQAPRLVIYDASSRA | 64D | (Human) | (Human) | (Human) | | GPGH | | QN | (https://science.science |
| | TGIPGRFSGSGAGTDFTLTINSLEPEDF | (Human) | | | | | | | | mag.org/content/early/ |
| | AVYYCQQRSNWPQNFGGGTKVEIK | | | | | | | | | 2020/06/15/science.abc |
| | | | | | | | | | | 7424) |
| mAb-20 | QSVLTQPPSVSGAPGQRVTISCTGSSS | IGHV4-59 | IGHJ2 | IGLV1-40 | IGLJ6 | 2990 | ARVGWVRYF | 4180 | QSYDSSLS | Anna Wec et al., 2020 |
| | NIGAGYYVHWYQQLPGTAPKLLIYDN | (Human) | (Human) | (Human) | (Human) | | DWSKPYYYFD | | AS | (https://science.science |
| | NNRPSGVPDRFSGSRSGTSASLAITGL | | | | | | L | | | mag.org/content/early/ |
| | QAEDEGDYFCQSYDSSLSASFGGGTK | | | | | | | | | 2020/06/15/science.abc |
| | VTVL | | | | | | | | | 7424) |
| mAb-21 | QSVLTQPPSVSGAPGQRVTISCTGSNS | IGHV3-23 | IGHJ5 | IGLV1-40 | IGLJ3 | 2991 | AKCDLVRYFD | 4181 | QSYDSSLS | Anna Wec et al., 2020 |
| | NIGAGYDVHWYQQLPETAPKLLIYAN | (Human) | (Human) | (Human) | (Human) | | WLGEENNWF | | GVL | (https://science.science |
| | GNRPSGVPDRFSGSKSGTSASLAITGL | | | | | | DP | | | mag.org/content/early/ |
| | QAEDEADYYCQSYDSSLSGVLFGGGT | | | | | | | | | 2020/06/15/science.abc |
| | KLTVL | | | | | | | | | 7424) |
| mAb-22 | DIQLTQSPLSLPVTLGQPASISCRSSQH | IGHV1-69 | IGHJ3 | IGKV2-30 | IGKJ1 | 2992 | VRDSDPYTAT | 4182 | MQGTEW | Anna Wec et al., 2020 |
| | LVHSDGNTYLNWFQQRPGQSPRRLIY | (Human) | (Human) | (Human) | (Human) | | VRNNHYWYA | | PRT | (https://science.science |
| | KVSNRDSGVPDRFSGSGSGIDFTLKISR | | | | | | LDV | | | mag.org/content/early/ |
| | VEAEDIGVYYCMQGTEWPRTFGQGT | | | | | | | | | 2020/06/15/science.abc |
| | KVEIK | | | | | | | | | 7424) |
| mAb-23 | DIQVTQSPATVSVSPGEGASLSCRASQ | IGHV3-66 | IGHJ4 | IGKV3-15 | IGKJ4 | 2993 | ARATPPGGG | 4183 | QQYNTWP | Anna Wec et al., 2020 |
| | SVRSNLAWFQQKPGQAPRLLISDAST | (Human) | (Human) | (Human) | (Human) | | TGWPYFDF | | PLT | (https://science.science |
| | RASGVPARFTGSGFGTEFTLTISSLQSE | | | | | | | | | mag.org/content/early/ |
| | DFAIYYCQQYNTWPPLTFGGGTKVEIK | | | | | | | | | 2020/06/15/science.abc |
| | | | | | | | | | | 7424) |
| mAb-24 | DIRLTQSPLSLPVTLGQPASISCRSSQSL | IGHV1-69 | IGHJ6 | IGKV2-30 | IGKJ4 | 2994 | AREEPSGTYH | 4184 | MQGTHW | Anna Wec et al., 2020 |
| | VHSNGNTYLNWFQQRPGQSPRRLIYE | (Human) | (Human) | (Human) | (Human) | | NYYGLDV | | PRS | (https://science.science |
| | VSNRDSGVPDRFTGSGSGTDFTLKISR | | | | | | | | | mag.org/content/early/ |
| | VEAEDVGVYYCMQGTHWPRSFGGG | | | | | | | | | 2020/06/15/science.abc |
| | TKVEIK | | | | | | | | | 7424) |
| mAb-25 | ETTLTQSPGTLSLSPGERATLSCRASQSI | IGHV4-4 | IGHJ3 | IGKV3-20 | IGKJ1 | 2995 | AARIRGATHY | 4185 | QQYGSAPL | Anna Wec et al., 2020 |
| | GSSYLAWYQQKPGQAPRLLIYGASSR | (Human) | (Human) | (Human) | (Human) | | DFWSGFWA | | YT | (https://science.science |
| | ATGIPDRFSGSGSGTDFTLTISRLEPED | | | | | | GPFDI | | | mag.org/content/early/ |
| | FAVYYCQQYGSAPLYTFGQGTKVEIK | | | | | | | | | 2020/06/15/science.abc |
| | | | | | | | | | | 7424) |
| mAb-26 | EIVLTQSPGTLSLSPGERASLSCRASQS | IGHV4-4 | IGHJ3 | IGKV3-20 | IGKJ3 | 2996 | ARTQSNDFW | 4186 | QQYGNSP | Anna Wec et al., 2020 |
| | VSSSYLAWYQHRPGQAPRLLIYDASSR | (Human) | (Human) | (Human) | (Human) | | SGYYTAAFDL | | RT | (https://science.science |
| | ATGIPDRFSGSGSGTDFTLTISRLESED | | | | | | | | | mag.org/content/early/ |
| | FAVYYCQQYGNSPRTFGQGTKVDIK | | | | | | | | | 2020/06/15/science.abc |
| | | | | | | | | | | 7424) |
| mAb-27 | QPVLTQPPSVSAAPGQKVTISCSGSSS | IGHV4-34 | IGHJ5 | IGLV1-51 | IGLJ3 | 2997 | ARGSLSREYD | 4187 | ETWDTSLS | Anna Wec et al., 2020 |
| | NIGDNYVSWYQQLPGTAPKLLIYDNN | (Human) | (Human) | (Human) | (Human) | | FLTAPQNGP | | VVV | (https://science.science |
| | KRPSGIPDRFSGSKSGTSATLGITGLQT | | | | | | WFDS | | | mag.org/content/early/ |
| | GDEADYYCETWDTSLSVVVFGGGTKL | | | | | | | | | 2020/06/15/science.abc |
| | TVL | | | | | | | | | 7424) |
| mAb-28 | DIVMTQTPLSLAVTLGQPASISCRSSH | IGHV1-69 | IGHJ6 | IGKV2-30 | IGKJ1 | 2998 | VLDTTSANPH | 4188 | MQGTDW | Anna Wec et al., 2020 |
| | GLVHTNGNTYLNWFHQRPGQPPRRL | (Human) | (Human) | (Human) | (Human) | | NWYGMDV | | PRT | (https://science.science |
| | IYKISNRDSGVPDRFSGSGSGTDFTLKI | | | | | | | | | mag.org/content/early/ |
| | SRVEADDVGVYYCMQGTDWPRTFG | | | | | | | | | 2020/06/15/science.abc |
| | QGTKVEIK | | | | | | | | | 7424) |
| mAb-29 | EIVLTQSPGTLSLSPGERATLSCRASQT | IGHV3-11 | IGHJ4 | IGKV3-20 | IGKJ2 | 2999 | ARMGPYGSG | 4189 | LQYSDATT | Anna Wec et al., 2020 |
| | VTRSQLAWYQHKPGQPPRLLIYDSSKR | (Human) | (Human) | (Human) | (Human) | | SFDY | | | (https://science.science |
| | ATGSPDRFSASGSGTDFTLTISGLEPED | | | | | | | | | mag.org/content/early/ |
| | TGIYYCLQYSDATTFGPGTKLEIK | | | | | | | | | 2020/06/15/science.abc |
| | | | | | | | | | | 7424) |
| mAb-3 | EIVMTQSPSSLSASIGDRVTISCRASQN | IGHV4-38- | IGHJ4 | IGKV1-39 | IGKJ5 | 3000 | AGRHQELLP | 4190 | QQSYTTPI | Anna Wec et al., 2020 |
| | IGSYLNWYQQRPGKAPNLLIFVASSLQ | 2 | (Human) | (Human) | (Human) | | MGSFDM | | T | (https://science.science |
| | SGVPSRFSGSGSGTDFTLTISSLQAEDF | (Human) | | | | | | | | mag.org/content/early/ |
| | ATYYCQQSYTTPITFGQGTRLEIK | | | | | | | | | 2020/06/15/science.abc |
| | | | | | | | | | | 7424) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| mAb-30 | Ab | SARS-CoV2 | 229E, | | | S; Unk | B-cells; SARS-CoV1 | 895 | PGPACTVWAEVKKPGSSVKVSCKASGG | 1962 |
| | | | HKU1, | | | | Human Patient | | MFSDYAISWVRQAPGQRLEWMGGIM | |
| | | | NL63, | | | | | | PGLGSPGYAQIFRGRATISADVSTSTAYL | |
| | | | OC43, | | | | | | ELTSLKPEDTAVYYCARDPSILNTGNHH | |
| | | | SARS- | | | | | | WYDLDIWGQGTQVTVSS | |
| | | | CoV1 | | | | | | | |
| mAb-31 | Ab | SARS-CoV2 | 229E, | | | S; Unk | B-cells; SARS-CoV1 | 896 | QVQLVQSGAEVKKPGSSVKVSCKASGG | 1963 |
| | | | HKU1, | | | | Human Patient | | TSSTHAISWVRQAPGQGLEWMGGIIPIF | |
| | | | NL63, | | | | | | GTTNYAQKFQDRVTITADESTSTAYMEL | |
| | | | OC43, | | | | | | SSLRSEDTAVYFCVRDGAYDSSGYYSTQ | |
| | | | SARS- | | | | | | WGQGTLVTVSS | |
| | | | CoV1 | | | | | | | |
| mAb-32 | Ab | SARS-CoV1, | 229E, | | | S; Unk | B-cells; SARS-CoV1 | 897 | EVQLVQSGAEVKKPGSSVKVSCKVSGG | 1964 |
| | | SARS-CoV2 | HKU1, | | | | Human Patient | | RFSDYAISWLRQAPVKGLEWMGGIIPRL | |
| | | | NL63, | | | | | | NRKGYSQDFQGRLTFTADESTSTAYMEL | |
| | | | OC43 | | | | | | SGLTSEDTAVYYCARDPTFLNSGNHFWY | |
| | | | | | | | | | AVDIWGQGTTVTVSS | |
| mAb-33 | Ab | SARS-CoV1, | 229E, | | | S; Unk | B-cells; SARS-CoV1 | 898 | QVQLVQSGAEVKKPGSSVTVSCKVSGG | 1965 |
| | | SARS-CoV2 | HKU1, | | | | Human Patient | | RFSDYAISWLRQAPVEGLEWMGGIIPHL | |
| | | | NL63, | | | | | | NKKGYSQKFQDRITFTADESTSTAYMEL | |
| | | | OC43 | | | | | | SGLTSEDTAIYYCARDPTFLNTGNHFWY | |
| | | | | | | | | | AVDIWGQGTTVTVSS | |
| mAb-34 | Ab | SARS-CoV1, | 229E, | | | S; Unk | B-cells; SARS-CoV1 | 899 | QVQLVQSGAEVKKPGASVKVSCKASGY | 1966 |
| | | SARS-CoV2 | HKU1, | | | | Human Patient | | TFSLFHVHWVRQAPGQGLEWMGWIN | |
| | | | NL63, | | | | | | PHNGDTTFAERFQGRVALTRDTSINTAY | |
| | | | OC43 | | | | | | MELSRLTSDDTAVYFCARDFGVRYDDSR | |
| | | | | | | | | | QLMKYCDSWGQGTLVTVSS | |
| mAb-35 | Ab | SARS-CoV1, | 229E, | | | S; Unk | B-cells; SARS-CoV1 | 900 | QVQLVQSGAEVKKPGSSVKVSCKASGG | 1967 |
| | | SARS-CoV2 | HKU1, | | | | Human Patient | | TLADYAISWVRQAPGQGLEWMGGIKP | |
| | | | NL63, | | | | | | LHGAAGYSQLFRGRLSITADESASTAYM | |
| | | | OC43 | | | | | | ELTGLTSDDTAMYYCARDPSILNTGNHH | |
| | | | | | | | | | WYDLDLWGQGTTVTVSS | |
| mAb-36 | Ab | SARS-CoV1, | 229E, | | | S; Unk | B-cells; SARS-CoV1 | 901 | QVQLVQSGPELKKPGSSVRVSCKASGGS | 1968 |
| | | SARS-CoV2 | HKU1, | | | | Human Patient | | ISSYAISWVRQAPGQRLEWMGGVLPM | |
| | | | NL63, | | | | | | MGRESPVQKFKDRVTIAADESTSTAYM | |
| | | | OC43 | | | | | | ELRSLSAEDTAVYYCVVDTTMADPHNW | |
| | | | | | | | | | YGLDVWGQGTTVTVSS | |
| mAb-37 | Ab | SARS-CoV1, | 229E, | | | S; Unk | B-cells; SARS-CoV1 | 902 | QVQLVQSGPGLVRPSGTLSLTCAVSGDS | 1969 |
| | | SARS-CoV2 | HKU1, | | | | Human Patient | | ISGDYWCTWVRQTPGKGLEWIGKISHS | |
| | | | NL63, | | | | | | GSINYNPSLKSRITMSVDKSKNQFSLKLN | |
| | | | OC43 | | | | | | SVTAADTAMYYCARVRVGASHHNFWS | |
| | | | | | | | | | GYYTDAFDIWGQGTTVTVSS | |
| mAb-38 | Ab | SARS-CoV1, | 229E, | | | S; Unk | B-cells; SARS-CoV1 | 903 | QVQLVQSGAEVKKPGSSVKVSCKVSGG | 1970 |
| | | SARS-CoV2 | HKU1, | | | | Human Patient | | TFSNYAISWLRQAPGQGPEWMGGIIPA | |
| | | | NL63, | | | | | | LSRVGYARKFQARLTISADELTTTAYMDL | |
| | | | OC43 | | | | | | SSLTSEDTAVYYCARDPSFLNTGNHFWY | |
| | | | | | | | | | DFDMWGQGTTVTVSS | |
| mAb-39 | Ab | SARS-CoV2 | 229E, | | | S; Unk | B-cells; SARS-CoV1 | 904 | EVQLQQSGAEVKKPGSSVKVSCKASGG | 1971 |
| | | | HKU1, | | | | Human Patient | | TSSTHAISWVRQAPGQGLEWMGGIIPIF | |
| | | | NL63, | | | | | | GTTNYAQKFQDRVTITADESTSTAYMEL | |
| | | | OC43, | | | | | | SSLRSEDTAVYFCVRDGAYDSSGYYSTQ | |
| | | | SARS- | | | | | | WGQGTLVTVSS | |
| | | | CoV1 | | | | | | | |
| mAb-4 | Ab | SARS-CoV1, | 229E, | | | S; Unk | B-cells; SARS-CoV1 | 905 | EVQLVESGGGLVKPGGSLRLSCAASGFT | 1972 |
| | | SARS-CoV2 | HKU1, | | | | Human Patient | | FSDYYMNWVRQAPGKGLEWVSSISSSS | |
| | | | NL63, | | | | | | YMYYADSMKGRFTISRDNAQNSLYLQM | |
| | | | OC43 | | | | | | SSLRAEDTAVYYCARDFPGDTAVAGTGF | |
| | | | | | | | | | NYWGQGTLVTVSS | |
| mAb-40 | Ab | SARS-CoV2 | 229E, | | | S; Unk | B-cells; SARS-CoV1 | 906 | QVQLVESGGDLVKPGRSLRLSCTASGFTI | 1973 |
| | | | HKU1, | | | | Human Patient | | GDYAMTWFRQAPGKGLECVAVIRSRAF | |
| | | | NL63, | | | | | | GGTTEYAASVKGRFIVSRDDSNSVAFLQ | |
| | | | OC43, | | | | | | MNSLKTEDTAVYYCSRDLRRGYYDSNG | |
| | | | SARS- | | | | | | HQQFDLWGQGTLVTVSS | |
| | | | CoV1 | | | | | | | |
| mAb-41 | Ab | SARS-CoV2 | 229E, | | | S; Unk | B-cells; SARS-CoV1 | 907 | QVQLVESGGGVVQPGRSLRLSCAASGF | 1974 |
| | | | HKU1, | | | | Human Patient | | TFSRYGMHWVRQAPGKGLEWVAVIYS | |
| | | | NL63, | | | | | | DGRNEYYADSVKGRFTISRDNSKNTLHL | |
| | | | OC43, | | | | | | QMNSLGAADTAVYYCARDPGPITFFDW | |
| | | | SARS- | | | | | | SPDKSRKSYYDYNGMDVWGQGTTVTV | |
| | | | CoV1 | | | | | | SS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| mAb-30 | DIVMTQSPLSLPVGLGQSASISCRSSQ | IGHV1-69 | IGHJ3 | IGKV2-30 | IGKJ1 | 3001 | ARDPSILNTG | 4191 | MQATEWP | Anna Wec et al., 2020 |
| | RVVHTDGNTYLNWFHQRPGQSPRRLI | (Human) | (Human) | (Human) | (Human) | | NHHWYDLDI | | RT | (https://science.science |
| | YKVSNRDSGVPDRFSGSGSGTDFTLRI | | | | | | | | | mag.org/content/early/ |
| | SRVEAEDVGVYYCMQATEWPRTFGQ | | | | | | | | | 2020/06/15/science.abc |
| | GTKVEIK | | | | | | | | | 7424) |
| mAb-31 | EIVLTQSPATLSLSPGERATLSCRASQS | IGHV1-69 | IGHJ4 | IGKV3-11 | IGKJ2 | 3002 | VRDGAYDSS | 4192 | QQRRNWP | Anna Wec et al., 2020 |
| | VSNFLAWYQQKPGQPPRLLIYDASNR | (Human) | (Human) | (Human) | (Human) | | GYYSTQ | | PMYT | (https://science.science |
| | ASGIAARFSGRGSGTDFTLTISSLEPED | | | | | | | | | mag.org/content/early/ |
| | FAVYYCQQRRNWPPMYTFGQGTKLEI | | | | | | | | | 2020/06/15/science.abc |
| | K | | | | | | | | | 7424) |
| mAb-32 | EIVLTQSPLSLSVTLGQAASISCTCSQTA | IGHV1-69 | IGHJ6 | IGKV2-30 | IGKJ2 | 3003 | ARDPTFLNSG | 4193 | MQTTDWP | Anna Wec et al., 2020 |
| | VHSDGNTYLNWFHQRPGQSPRRLIYK | (Human) | (Human) | (Human) | (Human) | | NHFWYAVDI | | RT | (https://science.science |
| | VSNRDSGVPDRFSGSGSGTDFTLKISR | | | | | | | | | mag.org/content/early/ |
| | VEAEDVGIYYCMQTTDWPRTFGQGT | | | | | | | | | 2020/06/15/science.abc |
| | KLEIK | | | | | | | | | 7424) |
| mAb-33 | ETTLTQSPLSLSVTLGQAASISCTCSQS | IGHV1-69 | IGHJ6 | IGKV2-30 | IGKJ3 | 3004 | ARDPTFLNTG | 4194 | MQTTDWP | Anna Wec et al., 2020 |
| | AVHSDGNTYFNWFHQRPGQSPRRLIY | (Human) | (Human) | (Human) | (Human) | | NHFWYAVDI | | RT | (https://science.science |
| | KVSNRDSGVPERFSGSGSGTHFTLIISR | | | | | | | | | mag.org/content/early/ |
| | VEAEDVGVYYCMQTTDWPRTFGQGT | | | | | | | | | 2020/06/15/science.abc |
| | KVDIK | | | | | | | | | 7424) |
| mAb-34 | NFMLTQPHSVSESPGKAVIISCTRSSG | IGHV1-2 | IGHJ5 | IGLV6-57 | IGLJ3 | 3005 | ARDFGVRYD | 4195 | QSYDSGNL | Anna Wec et al., 2020 |
| | NIASNFVQWYQQRPGSSPTPVIYEDKL | (Human) | (Human) | (Human) | (Human) | | DSRQLMKYC | | VV | (https://science.science |
| | RPSGVPDRFSGSIDRSSNSASLTISGLK | | | | | | DS | | | mag.org/content/early/ |
| | TEDEADYYCQSYDSGNLVVFGGGTKL | | | | | | | | | 2020/06/15/science.abc |
| | TVL | | | | | | | | | 7424) |
| mAb-35 | DIQMTQSPLSLPVTLGQAASISCRSSQ | IGHV1-69 | IGHJ6 | IGKV2-30 | IGKJ1 | 3006 | ARDPSILNTG | 4196 | MQGTEW | Anna Wec et al., 2020 |
| | PIPHTDGNTYLNWFHQRPGQSPRRLI | (Human) | (Human) | (Human) | (Human) | | NHHWYDLDL | | PRT | (https://science.science |
| | HKVSNRDSGVPDRFSGSGSGLDFTLEI | | | | | | | | | mag.org/content/early/ |
| | SGVEAEDVGIYYCMQGTEWPRTFGQ | | | | | | | | | 2020/06/15/science.abc |
| | GTKVEIK | | | | | | | | | 7424) |
| mAb-36 | EIVMTQTPLSLPVTLGQPASISCRSSHG | IGHV1-69 | IGHJ6 | IGKV2-30 | IGKJ3 | 3007 | VVDTTMADP | 4197 | MQGTDW | Anna Wec et al., 2020 |
| | LVHTNGNTYLNWFHQRPGQPPRRLIY | (Human) | (Human) | (Human) | (Human) | | HNWYGLDV | | PRT | (https://science.science |
| | QVSHRDSGVPDRFSGSGSGTDFTLKIS | | | | | | | | | mag.org/content/early/ |
| | RVEADDVGVYYCMQGTDWPRTFGQ | | | | | | | | | 2020/06/15/science.abc |
| | GTKVDIK | | | | | | | | | 7424) |
| mAb-37 | ETTLTQSPGTLSLSPGERATLSCRASQS | IGHV4-4 | IGHJ3 | IGKV3-20 | IGKJ1 | 3008 | ARVRVGASH | 4198 | QQYGTSPV | Anna Wec et al., 2020 |
| | VGNNYLAWYQQKPGQAPRLLIYGASS | (Human) | (Human) | (Human) | (Human) | | HNFWSGYYT | | YT | (https://science.science |
| | RATGIPDRFSGSGSGTDFTLTISRLEPE | | | | | | DAFDI | | | mag.org/content/early/ |
| | DFAVYYCQQYGTSPVYTFGQGTKVEIK | | | | | | | | | 2020/06/15/science.abc |
| | | | | | | | | | | 7424) |
| mAb-38 | DIQLTQSPLSLPVTLGQPASISCTSSQN | IGHV1-69 | IGHJ4 | IGKV2-30 | IGKJ1 | 3009 | ARDPSFLNTG | 4199 | MQGTDW | Anna Wec et al., 2020 |
| | VVHTDGNTYLNWFHQRPGQSPRRLIY | (Human) | (Human) | (Human) | (Human) | | NHFWYDFD | | PRT | (https://science.science |
| | KVSNRDSGVPDRFSGSGSGSHFTLKIS | | | | | | M | | | mag.org/content/early/ |
| | RVEAEDVGIYYCMQGTDWPRTFGQG | | | | | | | | | 2020/06/15/science.abc |
| | TKVEIK | | | | | | | | | 7424) |
| mAb-39 | DIVLTQTPATLSLSPGERATLSCRASQS | IGHV1-69 | IGHJ4 | IGKV3-11 | IGKJ1 | 3010 | VRDGAYDSS | 4200 | QQRRNWP | Anna Wec et al., 2020 |
| | VSNFLAWYQQKPGQPPRLLIYDASNR | (Human) | (Human) | (Human) | (Human) | | GYYSTQ | | PMYT | (https://science.science |
| | ASGIAARFSGRGSGTDFTLTISSLEPED | | | | | | | | | mag.org/content/early/ |
| | FAVYYCQQRRNWPPMYTFGQGTKVE | | | | | | | | | 2020/06/15/science.abc |
| | IK | | | | | | | | | 7424) |
| mAb-4 | QSVLTQPPSVSGAPGQRVTISCTGSSS | IGHV3-21 | IGHJ4 | IGLV1-40 | IGLJ1 | 3011 | ARDFPGDTA | 4201 | QSYDSSLS | Anna Wec et al., 2020 |
| | NIGAGYDVHWYQQVPGTAPKLLIYGN | (Human) | (Human) | (Human) | (Human) | | VAGTGFNY | | VLYV | (https://science.science |
| | SNRPSGVPDRFSGSKSGTSASLAITGL | | | | | | | | | mag.org/content/early/ |
| | QAEDEADYYCQSYDSSLSVLYVFGTGT | | | | | | | | | 2020/06/15/science.abc |
| | KVTVL | | | | | | | | | 7424) |
| mAb-40 | DIRLTQSPATLSLSPGERATLSCRASQSI | IGHV3-49 | IGHJ4 | IGKV3-11 | IGKJ1 | 3012 | SRDLRRGYYD | 4202 | QHRTNWP | Anna Wec et al., 2020 |
| | GYYLAWFQQKPGQAPRLLIYDASKRA | (Human) | (Human) | (Human) | (Human) | | SNGHQQFDL | | YT | (https://science.science |
| | TGIPARFSGSGSGTDFTLTISSLEPEDFA | | | | | | | | | mag.org/content/early/ |
| | VYYCQHRTNWPYTFGQGTKVEIK | | | | | | | | | 2020/06/15/science.abc |
| | | | | | | | | | | 7424) |
| mAb-41 | QPGLTQEPSLTVSPGGTVTLTCGSSAG | IGHV3-30 | IGHJ6 | IGLV7-46 | IGLJ3 | 3013 | ARDPGPITFF | 4203 | FLSYRGAP | Anna Wec et al., 2020 |
| | AVTSGHYPYWFQQKPGQAPRTLIYDT | (Human) | (Human) | (Human) | (Human) | | DWSPDKSRK | | PV | (https://science.science |
| | NNKYSWTPARFSGSLLGGKAALTLSG | | | | | | SYYDYNGMD | | | mag.org/content/early/ |
| | AQPEDEAEYFCFLSYRGAPPVFGGGT | | | | | | V | | | 2020/06/15/science.abc |
| | HLTVL | | | | | | | | | 7424) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| mAb-42 | Ab | SARS-CoV2 | 229E, | | | S; Unk | B-cells; SARS-CoV1 | 908 | EVQLLESGGGVVQPGTSLRLSCVASGFT | 1975 |
| | | | HKU1, | | | | Human Patient | | FSTFAMHWVRQAPGKGLEWVALISFDS | |
| | | | NL63, | | | | | | TNIRYANSVRGRFTISRDNSKNTLYLEVD | |
| | | | OC43, | | | | | | SLRIEDTGVYYCARDLPPLDYWGQGTLV | |
| | | | SARS- | | | | | | TVSS | |
| | | | CoV1 | | | | | | | |
| mAb-43 | Ab | SARS-CoV1, | 229E, | | | S; Unk | B-cells; SARS-CoV1 | 909 | QVQLVQSGAEVRKPGSSVKLSCKASGG | 1976 |
| | | SARS-CoV2 | HKU1, | | | | Human Patient | | TFSTHAISWVRQAPGQRPEWMGGIIPIF | |
| | | | NL63, | | | | | | GESKDTQKFQGRVTFTADESTTTAYMEL | |
| | | | OC43 | | | | | | RSLRSDDTAIYYCVRDSDPYTTTFSHNHY | |
| | | | | | | | | | WYAMDVWGQGTTVTVSS | |
| mAb-44 | Ab | SARS-CoV1, | 229E, | | | S; Unk | B-cells; SARS-CoV1 | 910 | QVQLVQSGGGLVQPGGSLRVSCSASGF | 1977 |
| | | SARS-CoV2 | HKU1, | | | | Human Patient | | TFSSFAMHWVRQAPGKGLEYVAGISDN | |
| | | | NL63, | | | | | | GHTTMYADSVKGRFTISRDNSKNTLYLQ | |
| | | | OC43 | | | | | | LSSLRPEDTAVYFCVKDNVILPGAIVRPQ | |
| | | | | | | | | | FDYWGQGTLVTVSS | |
| mAb-45 | Ab | SARS-CoV2 | 229E, | | | S; Unk | B-cells; SARS-CoV1 | 911 | QVQLVQSGAEVKKPGSSVKVSCKVSGG | 1978 |
| | | | HKU1, | | | | Human Patient | | MFSDYAISWVRQAPGQRLEWMGGIM | |
| | | | NL63, | | | | | | PGLGSPAYAQIFRGRVTISADISTSTAYLE | |
| | | | OC43, | | | | | | VTSLRPEDTAVYYCARDPSILNTGNHHW | |
| | | | SARS- | | | | | | YDLDMWGQGTTVTVSS | |
| | | | CoV1 | | | | | | | |
| mAb-46 | Ab | SARS-CoV1, | 229E, | | | S; Unk | B-cells; SARS-CoV1 | 912 | EVQLVESGGGVVQPGRSLRLSCVASGFT | 1979 |
| | | SARS-CoV2 | HKU1, | | | | Human Patient | | LAPYGMQWVRQAPGKGLEWVAFLSH | |
| | | | NL63, | | | | | | DGSHLGYVDSVKGRFTISRDNSKNTLYLE | |
| | | | OC43 | | | | | | MNSLRAEDTATYYCARDNVVQQNADN | |
| | | | | | | | | | VGYFDFWGQGSLVTVSS | |
| mAb-47 | Ab | SARS-CoV1, | 229E, | | | S; Unk | B-cells; SARS-CoV1 | 913 | QVQLVQSGAEVKKPGSSVKVSCKASGG | 1980 |
| | | SARS-CoV2 | HKU1, | | | | Human Patient | | MFRDYAISWLRQAPGQRLEWMGGIM | |
| | | | NL63, | | | | | | PAFGAPGYAQIFRGRATISADVSTTTAYL | |
| | | | OC43 | | | | | | ELTSLMPDDTAVYYCARDPSILNTGNHH | |
| | | | | | | | | | WYDLDLWGQGTTVTVSS | |
| mAb-48 | Ab | SARS-CoV1, | 229E, | | | S; Unk | B-cells; SARS-CoV1 | 914 | QVTLKESGAEVKKPGSSVKVSCKTSGGT | 1981 |
| | | SARS-CoV2 | HKU1, | | | | Human Patient | | FSTHAISWVRQAPGQGPEWMGGIIPLF | |
| | | | NL63, | | | | | | GTSQHAQRFQARVKFTADESTNTAYME | |
| | | | OC43 | | | | | | LSSLTPEDTAVYYCVRDSDPYTATSRNNH | |
| | | | | | | | | | YWYGMDVWGHGTTVTVSS | |
| mAb-49 | Ab | SARS-CoV1, | 229E, | | | S; Unk | B-cells; SARS-CoV1 | 915 | QVQLVQSGAEVKKPGSSVKVSCKASGG | 1982 |
| | | SARS-CoV2 | HKU1, | | | | Human Patient | | MFSDYAISWVRQAPGQRLEWMGGIM | |
| | | | NL63, | | | | | | PGLGSPAYAQSFGGRVTISADISTSTAYL | |
| | | | OC43 | | | | | | EVTSLRPEDTAVYYCARDPSILNTGNHH | |
| | | | | | | | | | WYDLDMWGQGTMVTVSS | |
| mAb-5 | Ab | SARS-CoV1, | 229E, | | | S; Unk | B-cells; SARS-CoV1 | 916 | EVQLVESGGGLVQPGRSLRLSCSASGFS | 1983 |
| | | SARS-CoV2 | HKU1, | | | | Human Patient | | FGDYAMSWFRQAPGKGLQWVGLIKTR | |
| | | | NL63, | | | | | | AYGAATDYAASVQGRFIISRDDSKSIAYL | |
| | | | OC43 | | | | | | QMNSLKTEDTAVYFCAREGTSLGYYYYY | |
| | | | | | | | | | AMDVWGHGTTVTVSS | |
| mAb-50 | Ab | SARS-CoV1, | 229E, | | | S; Unk | B-cells; SARS-CoV1 | 917 | QVQLVQSGAEVKKPGSSVKVSCKASGG | 1984 |
| | | SARS-CoV2 | HKU1, | | | | Human Patient | | MFSDYAISWVRQAPGQRLEWMGGIM | |
| | | | NL63, | | | | | | PGLGSPAYAQIFRGRVTISADISTSTAYLE | |
| | | | OC43 | | | | | | VTSLRPEDTAVYYCARDPSILNTGNHHW | |
| | | | | | | | | | YDLDMWGQGTTVTVSS | |
| mAb-51 | Ab | SARS-CoV1, | 229E, | | | S; Unk | B-cells; SARS-CoV1 | 918 | EVQLVESGGGLVQPGGSLRLSCLTSGFS | 1985 |
| | | SARS-CoV2 | HKU1, | | | | Human Patient | | FSSYWMIWVRQAPGKGLEWVANIEPD | |
| | | | NL63, | | | | | | GNEEYYVDSVKGRFTISRDNAKNSLYLQ | |
| | | | OC43 | | | | | | MNSLRAEDTAVYYCARGPIRHFGLDAFD | |
| | | | | | | | | | IWGQGTTVTVSS | |
| mAb-52 | Ab | SARS-CoV2 | 229E, | | | S; Unk | B-cells; SARS-CoV1 | 919 | QVQLVQSGDEMKKPGSSVKVSCKASGD | 1986 |
| | | | HKU1, | | | | Human Patient | | TFSTHAISWVRQAPGQGPEWMGGIIPL | |
| | | | NL63, | | | | | | FGTASYAQTSQSRVKITADESTSTAYMEL | |
| | | | OC43, | | | | | | SSLTSEDTAVYYCVRDSDPYTATSRNNHY | |
| | | | SARS- | | | | | | WYGMDVWGQGTTVTVSS | |
| | | | CoV1 | | | | | | | |
| mAb-53 | Ab | SARS-CoV1, | 229E, | | | S; Unk | B-cells; SARS-CoV1 | 920 | QVQLVQSGAEVKKPGSSVKVSCKASGG | 1987 |
| | | SARS-CoV2 | HKU1, | | | | Human Patient | | MFSDYAISWVRQAPGQRLEWMGGIM | |
| | | | NL63, | | | | | | PGLGSPAYAQSFGGRVTISADISTSTAYL | |
| | | | OC43 | | | | | | EVTSLRPEDTAVYYCARDPSILNTGNHH | |
| | | | | | | | | | WYDLDMWGQGTMVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| mAb-42 | DIRMTQSPFSLSASVGDRVTITCRASQ | IGHV3-30 | IGHJ4 | IGKV1-27 | IGKJ1 | 3014 | ARDLPPLDY | 4204 | QKHDRDP | Anna Wec et al., 2020 |
| | GFGNRLAWYQQKPGRAPKLLIYDAST | (Human) | (Human) | (Human) | (Human) | | | | WT | (https://science.science |
| | LQSGVPSRFSGSGSETDFALTISSLQPE | | | | | | | | | mag.org/content/early/ |
| | DVATYYCQKHDRDPWTFGQGTKVEIK | | | | | | | | | 2020/06/15/science.abc |
| | | | | | | | | | | 7424) |
| mAb-43 | DIVMTQSPLSLPVTLGQPASISCRSSQR | IGHV1-69 | IGHJ6 | IGKV2-30 | IGKJ1 | 3015 | VRDSDPYTTT | 4205 | MQGTEW | Anna Wec et al., 2020 |
| | LVHSDGNTYLNWFQQRPGQSPRRLIY | (Human) | (Human) | (Human) | (Human) | | FSHNHYWYA | | PRT | (https://science.science |
| | KVSNRDSGVPDRFSGSGSGTDFTLKIS | | | | | | MDV | | | mag.org/content/early/ |
| | RVEAEDIGVYYCMQGTEWPRTFGQG | | | | | | | | | 2020/06/15/science.abc |
| | TKVEIK | | | | | | | | | 7424) |
| mAb-44 | ETTLTQSPASVSASVGDRVTISCRASQ | IGHV3- | IGHJ4 | IGKV1-12 | IGKJ4 | 3016 | VKDNVILPGA | 4206 | QQAESFPF | Anna Wec et al., 2020 |
| | GIGRWLAWYQQKPGRAPKLLIFSASSL | 64D | (Human) | (Human) | (Human) | | IVRPQFDY | | T | (https://science.science |
| | QSGVPSRFSGSGSGTDFTLTISSLQPED | (Human) | | | | | | | | mag.org/content/early/ |
| | FATYYCQQAESFPFTFGGGTKVEIK | | | | | | | | | 2020/06/15/science.abc |
| | | | | | | | | | | 7424) |
| mAb-45 | ETTLTQSPLSLPVDLGQSASISCRSSQR | IGHV1-69 | IGHJ6 | IGKV2-30 | IGKJ1 | 3017 | ARDPSILNTG | 4207 | MQATDW | Anna Wec et al., 2020 |
| | VVHTNGNTYLHWFHQRPGQAPRRLI | (Human) | (Human) | (Human) | (Human) | | NHHWYDLD | | PRT | (https://science.science |
| | YKVSNRESGVPDRFSGSGSGTDFTLRI | | | | | | M | | | mag.org/content/early/ |
| | SRVEAEDVGVYYCMQATDWPRTFGQ | | | | | | | | | 2020/06/15/science.abc |
| | GTKVEIK | | | | | | | | | 7424) |
| mAb-46 | CIRLTQSPATLSLSPGERATLSCRASQS | IGHV3-30 | IGHJ4 | IGKV3-11 | IGKJ3 | 3018 | ARDNVVQQN | 4208 | QQRGNGY | Anna Wec et al., 2020 |
| | VTSYLAWYQQRPGQAPRLLIYDTSNR | (Human) | (Human) | (Human) | (Human) | | ADNVGYFDF | | T | (https://science.science |
| | VTGVPVRFSGSGYGTDFTLTISSLEPED | | | | | | | | | mag.org/content/early/ |
| | FAVYYCQQRGNGYTFGPGTKVEIK | | | | | | | | | 2020/06/15/science.abc |
| | | | | | | | | | | 7424) |
| mAb-47 | ETTLTQSPLSLPVGLGQSASISCRSSQR | IGHV1-69 | IGHJ6 | IGKV2-30 | IGKJ3 | 3019 | ARDPSILNTG | 4209 | MQATEWP | Anna Wec et al., 2020 |
| | VVHTDGNTYLNWFHQRPGQSPRRLIY | (Human) | (Human) | (Human) | (Human) | | NHHWYDLDL | | RT | (https://science.science |
| | KVSNRDSGVPDRFSGSGSGTDFTLRIS | | | | | | | | | mag.org/content/early/ |
| | RVEAEDVGVYYCMQATEWPRTFGQG | | | | | | | | | 2020/06/15/science.abc |
| | TKVDIK | | | | | | | | | 7424) |
| mAb-48 | EIVMTQSPLSLPVTLGQPASISCRSSQS | IGHV1-69 | IGHJ6 | IGKV2-30 | IGKJ2 | 3020 | VRDSDPYTAT | 4210 | MQGTDW | Anna Wec et al., 2020 |
| | VVHSDGNTYLNWFHQRPGQSPRRLIY | (Human) | (Human) | (Human) | (Human) | | SRNNHYWYG | | PRT | (https://science.science |
| | KVSNRDSGVPDRFSGSGSGTDFTLKIS | | | | | | MDV | | | mag.org/content/early/ |
| | RVEAEDIGVYYCMQGTDWPRTFGQG | | | | | | | | | 2020/06/15/science.abc |
| | TKLEIK | | | | | | | | | 7424) |
| mAb-49 | DIVMTQSPLSLPVDLGQSASISCRSSQT | IGHV1-69 | IGHJ3 | IGKV2-30 | IGKJ2 | 3021 | ARDPSILNTG | 4211 | MQATDW | Anna Wec et al., 2020 |
| | AVHTNGNTYLHWFHQRPGQAPRRLI | (Human) | (Human) | (Human) | (Human) | | NHHWYDLD | | PRT | (https://science.science |
| | YKVSNRESGVPDRFSGSGSGTDFTLRI | | | | | | M | | | mag.org/content/early/ |
| | NRVEAEDVGVYYCMQATDWPRTFG | | | | | | | | | 2020/06/15/science.abc |
| | QGTKLEIK | | | | | | | | | 7424) |
| mAb-5 | DIVLTQTPSSLSASVGDRVTITCRASQTI | IGHV3-49 | IGHJ6 | IGKV1-39 | IGKJ3 | 3022 | AREGTSLGYY | 4212 | QQSYSVPL | Anna Wec et al., 2020 |
| | SYYLNWYQQKVGKAPQLLVYAASSLQ | (Human) | (Human) | (Human) | (Human) | | YYYAMDV | | T | (https://science.science |
| | SGVPSRFSGSGSGTDFTLTISSLQPEDF | | | | | | | | | mag.org/content/early/ |
| | ATYYCQQSYSVPLTLGQGTKVDIK | | | | | | | | | 2020/06/15/science.abc |
| | | | | | | | | | | 7424) |
| mAb-50 | DIQMTQSPLSLPVDLGQSASISCRSSQ | IGHV1-69 | IGHJ6 | IGKV2-30 | IGKJ1 | 3023 | ARDPSILNTG | 4213 | MQATDW | Anna Wec et al., 2020 |
| | RVVHTNGNTYLHWFHQRPGQAPRRL | (Human) | (Human) | (Human) | (Human) | | NHHWYDLD | | PRT | (https://science.science |
| | IYKVSNRESGVPDRFSGSGSGTDFTLRI | | | | | | M | | | mag.org/content/early/ |
| | SRVEAEDVGVYYCMQATDWPRTFGQ | | | | | | | | | 2020/06/15/science.abc |
| | GTKVEIK | | | | | | | | | 7424) |
| mAb-51 | QPGLTQPASVSGSPGQSITISCTRTSG | IGHV3-7 | IGHJ3 | IGLV2-14 | IGLJ3 | 3024 | ARGPIRHFGL | 4214 | STYTSTSTI | Anna Wec et al., 2020 |
| | DVGDYNSVSWYVSWYQQHPGRAPKL | (Human) | (Human) | (Human) | (Human) | | DAFDI | | | (https://science.science |
| | MLYDVSNRPSGVSNRFSGSKLGDTAS | | | | | | | | | mag.org/content/early/ |
| | LTISELQAEDEADYYCSTYTSTSTIFGGG | | | | | | | | | 2020/06/15/science.abc |
| | TKLTVL | | | | | | | | | 7424) |
| mAb-52 | DIQMTQSPLSLPVTLGQPASISCRASQ | IGHV1-69 | IGHJ6 | IGKV2-30 | IGKJ3 | 3025 | VRDSDPYTAT | 4215 | MQGTDW | Anna Wec et al., 2020 |
| | TVVHTNGNTYLNWFHQRPGQSPRRLI | (Human) | (Human) | (Human) | (Human) | | SRNNHYWYG | | PRT | (https://science.science |
| | YEVSNRDSGVPDRFSGSGSGTDFTLSI | | | | | | MDV | | | mag.org/content/early/ |
| | SRVEAEDIGVYYCMQGTDWPRTFGP | | | | | | | | | 2020/06/15/science.abc |
| | GTKVEIK | | | | | | | | | 7424) |
| mAb-53 | DIRLTQSPLSLPVDLGQSASISCRSSQT | IGHV1-69 | IGHJ3 | IGKV2-30 | IGKJ3 | 3026 | ARDPSILNTG | 4216 | MQATDW | Anna Wec et al., 2020 |
| | VVHTNGNTYLHWFHQRPGQAPRRLI | (Human) | (Human) | (Human) | (Human) | | NHHWYDLD | | PRT | (https://science.science |
| | YKVSNRESGVPDRFSGSGSGTDFTLRI | | | | | | M | | | mag.org/content/early/ |
| | SRVEAEDVGVYYCMQATDWPRTFGQ | | | | | | | | | 2020/06/15/science.abc |
| | GTKVDIK | | | | | | | | | 7424) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| mAb-54 | Ab | SARS-CoV1, | 229E, | | | S; Unk | B-cells; SARS-CoV1 | 921 | EVQLVESGGGLVQPGGSLRLSCSASGFT | 1988 |
| | | SARS-CoV2 | HKU1, | | | | Human Patient | | FSNYAMYWVRQAPGKRPEYVSGISSNG | |
| | | | NL63, | | | | | | GITYYADSVEGRFTVSRDNSKKSLYLQM | |
| | | | OC43 | | | | | | SSLRPEDTAVYYCVKDLGATVTYDVFDV | |
| | | | | | | | | | WGQGTMVTVSS | |
| mAb-55 | Ab | SARS-CoV2 | 229E, | | | S; Unk | B-cells; SARS-CoV1 | 922 | EVQLVESGGGLVQPGGSLRLSCAASGFT | 1989 |
| | | | HKU1, | | | | Human Patient | | FSSYSMSWVRQAPGKGLEWVSGHDGG | |
| | | | NL63, | | | | | | TTHYADSVKGRFTISRDDSMNTLSLQM | |
| | | | OC43, | | | | | | NSLRAEDTAVYYCAKERDLPGRGGYFDH | |
| | | | SARS- | | | | | | WGQGTLVTVSS | |
| | | | CoV1 | | | | | | | |
| mAb-56 | Ab | SARS-CoV2 | 229E, | | | S; Unk | B-cells; SARS-CoV1 | 923 | QVQLQQWGAGLLKPSETLSLTCAVYGG | 1990 |
| | | | HKU1, | | | | Human Patient | | SFRGFYWTWIRQPPGKGLEWIGEVSHS | |
| | | | NL63, | | | | | | GETNYNPSLKSRVTISVDTSKNQFSLNLIS | |
| | | | OC43, | | | | | | VTAADTSVYYCARGYTAPIIREVPITFRPR | |
| | | | SARS- | | | | | | WFDPWGQGTPVTVSS | |
| | | | CoV1 | | | | | | | |
| mAb-57 | Ab | SARS-CoV1, | 229E, | | | S; Unk | B-cells; SARS-CoV1 | 924 | QVQLVQSGGGVVNPGGSMRLSCAGSG | 1991 |
| | | SARS-CoV2 | HKU1, | | | | Human Patient | | FTFSDHYMGWIRQAPGKGLEVISYISSS | |
| | | | NL63, | | | | | | GSFIRDADSVKGRFTISRDNAKNSVYLQ | |
| | | | OC43 | | | | | | MNSLRAEDTAVYYCARMGPYGSGTEDY | |
| | | | | | | | | | WGQGTLVTVSS | |
| mAb-58 | Ab | SARS-CoV1, | 229E, | | | S; Unk | B-cells; SARS-CoV1 | 925 | QVQLVQSGGNVVQPGGSLRLSCVGSEF | 1992 |
| | | SARS-CoV2 | HKU1, | | | | Human Patient | | SITFFAMQWVRRTPGKGLEWVALVSHD | |
| | | | NL63, | | | | | | GSNIRYSDSVKGRFIISRDNAKNTLYLQL | |
| | | | OC43 | | | | | | DSLTPEDTGIYYCARDHALQNGRPGYFD | |
| | | | | | | | | | SWGQGSQVTVSS | |
| mAb-59 | Ab | SARS-CoV1, | 229E, | | | S; Unk | B-cells; SARS-CoV1 | 926 | QVQLVQSGGGLVQPGGSLRLSCSASGF | 1993 |
| | | SARS-CoV2 | HKU1, | | | | Human Patient | | TFNTYAMHWVRQAPGKRLEYVSSITRD | |
| | | | NL63, | | | | | | GAGKFYADSVKGRFTISRDNSKNTLYQQ | |
| | | | OC43 | | | | | | MSSLRPEDTAVYYCVREGQQWLGLYFD | |
| | | | | | | | | | HWGQGALVTVSS | |
| mAb-6 | Ab | SARS-CoV1, | 229E, | | | S; Unk | B-cells; SARS-CoV1 | 927 | QVQLVQSGAEVKKPGASVKVSCRASGY | 1994 |
| | | SARS-CoV2 | HKU1, | | | | Human Patient | | TFSGYDINWVRQATGQGLEWMGWM | |
| | | | NL63, | | | | | | NPNSGDTGYAHKFQGRVTMTRNSSIST | |
| | | | OC43 | | | | | | AYMELSSLTSEDTAVYFCAREKKSFGPQY | |
| | | | | | | | | | YYGSGEDWGQGTLVTVSS | |
| mAb-60 | Ab | SARS-CoV1, | 229E, | | | S; Unk | B-cells; SARS-CoV1 | 928 | EVQLVQSGAEMKKPGESLKISCKGSGYS | 1995 |
| | | SARS-CoV2 | HKU1, | | | | Human Patient | | FPNYWIGWVRQMPGKGLEWMAIMW | |
| | | | NL63, | | | | | | PSDSDTRYSPSFQGQVTISADTSTSTVYL | |
| | | | OC43 | | | | | | QWGSLKASDTAMYYCVRQRYCSGGSCF | |
| | | | | | | | | | LFEDAFEIWGQGTMVTVSS | |
| mAb-61 | Ab | SARS-CoV2 | 229E, | | | S; Unk | B-cells; SARS-CoV1 | 929 | EVQLVESGGGLVQPGGSLRLSCAASGFI | 1996 |
| | | | HKU1, | | | | Human Patient | | FKNEPMNWIRQFPGKGLEWISNIRDNG | |
| | | | NL63, | | | | | | NDVYYADSVKGRFTVSRDNAKNSLYLQ | |
| | | | OC43, | | | | | | MNSLRDDDTALYYCVRDTDWAFDSWG | |
| | | | SARS- | | | | | | QGTLVTVSS | |
| | | | CoV1 | | | | | | | |
| mAb-62 | Ab | SARS-CoV2 | 229E, | | | S; Unk | B-cells; SARS-CoV1 | 930 | EVQLVESGPTLVKPTQTLTLTCTFSGFSLT | 1997 |
| | | | HKU1, | | | | Human Patient | | TRRQGVGWLRRPPGKALEWLALIYWD | |
| | | | NL63, | | | | | | DDKRYSPSLKSRLTITKDTSKNHVVLSLT | |
| | | | OC43, | | | | | | NVGPADTATYYCAHTSELPPRRPYAAFD | |
| | | | SARS- | | | | | | FWGQGTLVTVSS | |
| | | | CoV1 | | | | | | | |
| mAb-63 | Ab | SARS-CoV2 | 229E, | | | S; Unk | B-cells; SARS-CoV1 | 931 | EVQLLESGGGLVQPGGSLRLSCAASGFP | 1998 |
| | | | HKU1, | | | | Human Patient | | FSTDAMNWVRQAPGEGLEWVSTISDS | |
| | | | NL63, | | | | | | GRDTYYAASVRGRFTISRDNSKNTVYLQ | |
| | | | OC43, | | | | | | MNSLRVEDTAVYYCANTNFLDYWGQG | |
| | | | SARS- | | | | | | TLVTVSS | |
| | | | CoV1 | | | | | | | |
| mAb-64 | Ab | SARS-CoV1, | 229E, | | | S; Unk | B-cells; SARS-CoV1 | 932 | EVQLQESGPGLLKPSETLSLTCTVSGSPIA | 1999 |
| | | SARS-CoV2 | HKU1, | | | | Human Patient | | SNYWSWVRQPAGKGLEWIGRIDTSPTT | |
| | | | NL63, | | | | | | DYNPSLKSRVIMSVDTSTSQFSLKMSSVT | |
| | | | OC43 | | | | | | AADTAVYYCTRSFISFDSSGHPYYYYAMD | |
| | | | | | | | | | VWGQGTTVTVSS | |
| mAb-65 | Ab | SARS-CoV1, | 229E, | | | S; Unk | B-cells; SARS-CoV1 | 933 | QVQLVQSGAEVRKPGSSVKLSCRASGG | 2000 |
| | | SARS-CoV2 | HKU1, | | | | Human Patient | | TFHTYTVNWVRQAPGQGLEWLGGIIPIF | |
| | | | NL63, | | | | | | GTPTYAQRFQGKVSITADSSTNTVFMEL | |
| | | | OC43 | | | | | | TSLTSEDTAVYYCTRETGTDEFDFWGQG | |
| | | | | | | | | | ALVTVSS | |
| mAb-66 | Ab | SARS-CoV2 | 229E, | | | S; Unk | B-cells; SARS-CoV1 | 934 | EVQLVESGPGLVKPSGTLSLTCAVSGAS | 2001 |
| | | | HKU1, | | | | Human Patient | | VSSDHWWSWVRQSPGKGLEWIGEVY | |
| | | | NL63, | | | | | | HSGSTNYNPSLKSRVTISLDQSNNQFSLK | |
| | | | OC43, | | | | | | LTSVTAADTAIYYCATMWGGLCTASNCY | |
| | | | SARS- | | | | | | GNPMDVWGQGTTVTVSS | |
| | | | CoV1 | | | | | | | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| mAb-54 | DIRVTQSPSSLSATVGDRVTITCRASQT | IGHV3- | IGHJ3 | IGKV1-39 | IGKJ5 | 3027 | VKDLGATVTY | 4217 | QQTYITPG | Anna Wec et al., 2020 |
| | ITKYLNWYQQKPGKAPKLLLYGASSLQ | 64D | (Human) | (Human) | (Human) | | DVFDV | | T | (https://science.science |
| | SGVPSRFSGSGSGTDFTLTISSLQPEDF | (Human) | | | | | | | | mag.org/content/early/ |
| | ATYYCQQTYITPGTFGQGTRLEIK | | | | | | | | | 2020/06/15/science.abc |
| | | | | | | | | | | 7424) |
| mAb-55 | DIRLTQSPDSLALSLGERATINCKSSQS | IGHV3-23 | IGHJ4 | IGKV4-1 | IGKJ1 | 3028 | AKERDLPGRG | 4218 | QQYYLTP | Anna Wec et al., 2020 |
| | VLFSSNNNNYLGWYQQKPGQPPKLLI | (Human) | (Human) | (Human) | (Human) | | GYFDH | | WT | (https://science.science |
| | YWASTRESGVPDRFSGSGSGTDFTLTI | | | | | | | | | mag.org/content/early/ |
| | SSLQAEDVAVYYCQQYYLTPWTFGQG | | | | | | | | | 2020/06/15/science.abc |
| | TKVDIK | | | | | | | | | 7424) |
| mAb-56 | QPVLTQPASVSGSPGQSITISCTGTSSD | IGHV4-34 | IGHJ5 | IGLV2-23 | IGLJ3 | 3029 | ARGYTAPIIR | 4219 | CSYAVSGT | Anna Wec et al., 2020 |
| | VGNYNVVSWYQQHPGKAPQLFIYED | (Human) | (Human) | (Human) | (Human) | | EVPITFRPRW | | VL | (https://science.science |
| | TKRPSGVSDRFSGSKSGITASLTISRLQ | | | | | | FDP | | | mag.org/content/early/ |
| | PEDEADYYCCSYAVSGTVLFGGGTKVT | | | | | | | | | 2020/06/15/science.abc |
| | VL | | | | | | | | | 7424) |
| mAb-57 | EIVLTQSPGTLSVSPGERATLSCRASQII | IGHV3-11 | IGHJ4 | IGKV3-20 | IGKJ3 | 3030 | ARMGPYGSG | 4220 | LQYSLATT | Anna Wec et al., 2020 |
| | NRSQLGWYQHKPGQAPRLLIFDSSKR | (Human) | (Human) | (Human) | (Human) | | TFDY | | | (https://science.science |
| | ATGTPDRFSASGSETDFTLTISGVEPED | | | | | | | | | mag.org/content/early/ |
| | SGVYYCLQYSLATTFGPGTKVDIK | | | | | | | | | 2020/06/15/science.abc |
| | | | | | | | | | | 7424) |
| mAb-58 | DIQLTQSPATLSLSPGERATLSCRASPS | IGHV3-30 | IGHJ5 | IGKV3-11 | IGKJ3 | 3031 | ARDHALQNG | 4221 | QQRSDGY | Anna Wec et al., 2020 |
| | VFTFLAWYQQRPGQPPRLLIHDVSNR | (Human) | (Human) | (Human) | (Human) | | RPGYFDS | | N | (https://science.science |
| | APGIPARFSGSGSGTDFTLIISSLEPDDS | | | | | | | | | mag.org/content/early/ |
| | AVYFCQQRSDGYNFGPGTKVDIK | | | | | | | | | 2020/06/15/science.abc |
| | | | | | | | | | | 7424) |
| mAb-59 | DIRVTQSPSSLSASVGDRVTITCRASQT | IGHV3-64 | IGHJ4 | IGKV1-39 | IGKJ1 | 3032 | VREGQQWLG | 4222 | QQTYITPT | Anna Wec et al., 2020 |
| | INNYLNWYQQKPGKAPNLLIYAASTLQ | (Human) | (Human) | (Human) | (Human) | | LYFDH | | WT | (https://science.science |
| | NGVPSRFSGSGSGTDFTLTISSVQPED | | | | | | | | | mag.org/content/early/ |
| | FATYYCQQTYITPTWTFGQGTKVEIK | | | | | | | | | 2020/06/15/science.abc |
| | | | | | | | | | | 7424) |
| mAb-6 | EIVMTQSPSSLSASVGDRVTITCRASQS | IGHV1-8 | IGHJ4 | IGKV1-39 | IGKJ3 | 3033 | AREKKSFGPQ | 4223 | QQSYSTPY | Anna Wec et al., 2020 |
| | ISNYLYWYQQKPGKAPKLLIYVASNLQ | (Human) | (Human) | (Human) | (Human) | | YYYGSGED | | N | (https://science.science |
| | SGVPSRFSGSGSGTDFTLTISSLQPEDF | | | | | | | | | mag.org/content/early/ |
| | ATYYCQQSYSTPYNFGQGTKVDIK | | | | | | | | | 2020/06/15/science.abc |
| | | | | | | | | | | 7424) |
| mAb-60 | EIVLTQSPSTLSASVGDRVTITCRASQN | IGHV5-51 | IGHJ3 | IGKV1-5 | IGKJ3 | 3034 | VRQRYCSGGS | 4224 | QQYNSQY | Anna Wec et al., 2020 |
| | VNNWLAWYQQKPGKAPKLLIYEASTL | (Human) | (Human) | (Human) | (Human) | | CFLFEDAFEI | | T | (https://science.science |
| | KSGVPSRFSGSGSGTEFTLTISSLQPDD | | | | | | | | | mag.org/content/early/ |
| | FATYYCQQYNSQYTFGQGTKVDIK | | | | | | | | | 2020/06/15/science.abc |
| | | | | | | | | | | 7424) |
| mAb-61 | QPVLTQPRSVSGSPGQSVTISCTGSSST | IGHV3-48 | IGHJ5 | IGLV2-11 | IGLJ3 | 3035 | VRDTDWAFD | 4225 | CSSPGTIT | Anna Wec et al., 2020 |
| | FGTDNHISWYQQLPGKVPKLIIHAVSQ | (Human) | (Human) | (Human) | (Human) | | S | | WV | (https://science.science |
| | RPSVVPDRFSGSRSGNTASLTISGLQA | | | | | | | | | mag.org/content/early/ |
| | EDEADYYCCSSPGTITWVFGGGTKVT | | | | | | | | | 2020/06/15/science.abc |
| | VL | | | | | | | | | 7424) |
| mAb-62 | QSVLTQPPSVSGVPRQKVTISCSGSTA | IGHV2-5 | IGHJ4 | IGLV1-36 | IGLJ3 | 3036 | AHTSELPPRR | 4226 | ATWDDILN | 2020/06/15/science.abc |
| | NIASNGVNWYQLVPGKAPRLLISYDDL | (Human) | (Human) | (Human) | (Human) | | PYAAFDF | | GPV | Anna Wec et al., 2020 |
| | VPSGVSARFSGSKSGTSASLAISGLQAE | | | | | | | | | (https://science.science |
| | DEADYYCATWDDILNGPVFGGGTKLT | | | | | | | | | mag.org/content/early/ |
| | VL | | | | | | | | | 7424) |
| mAb-63 | DIVLTQSPLSLPVTLGQSASISCRSSQG | IGHV3-23 | IGHJ4 | IGKV2-30 | IGKJ2 | 3037 | ANTNFLDY | 4227 | MQATHW | Anna Wec et al., 2020 |
| | LVHSDGNIYLNWFQQRPGQSPRRLIY | (Human) | (Human) | (Human) | (Human) | | | | PRA | (https://science.science |
| | KVSNRDSGVPDRFSGSGSGTDFTLKIS | | | | | | | | | mag.org/content/early/ |
| | RVEAEDVGLYYCMQATHWPRAFGQ | | | | | | | | | 2020/06/15/science.abc |
| | GTKLEIK | | | | | | | | | 7424) |
| mAb-64 | EIVMTQSPGTLSLSPGERVTLSCRATQ | IGHV4-4 | IGHJ6 | IGKV3-20 | IGKJ1 | 3038 | TRSFISFDSS | 4228 | HQYGSSP | Anna Wec et al., 2020 |
| | SVSNNHLAWYQQKPGQAPRLLIYGAS | (Human) | (Human) | (Human) | (Human) | | GHPYYYYAMD | | WT | (https://science.science |
| | TTATDIPDRFSGRVAGTDFTLTISRLDP | | | | | | V | | | mag.org/content/early/ |
| | EDFAVYYCHQYGSSPWTFGQGTKVEI | | | | | | | | | 2020/06/15/science.abc |
| | K | | | | | | | | | 7424) |
| mAb-65 | DIRLTQSPATLSLSPGERATLSCRASQS | (Human) | IGHJ4 | IGKV3-11 | IGKJ1 | 3039 | TRETGTDEFD | 4229 | QHRSNWP | Anna Wec et al., 2020 |
| | VSSSLAWYQQKPGQAPRLLIYEASNRA | IGHV1-69 | (Human) | (Human) | (Human) | | F | | PRYT | (https://science.science |
| | TGVPARFSGSGSGTDFTLAISSLEPEDF | | | | | | | | | mag.org/content/early/ |
| | AVYYCQHRSNWPPRYTFGQGTKVEIK | | | | | | | | | 2020/06/15/science.abc |
| | | | | | | | | | | 7424) |
| mAb-66 | SYELTQPPSVSVSPGQTARITCSGDALP | IGHV4-4 | IGHJ6 | IGLV3-10 | IGLJ6 | 3040 | ATMWGGLCT | 4230 | YSTDSTAN | Anna Wec et al., 2020 |
| | RRYAYWYQQRSGQAPVLVIYEDNKRP | (Human) | (Human) | (Human) | (Human) | | ASNCYGNPM | | YKV | (https://science.science |
| | SGIPERFSAFSSGTRATLTISGAQVEDE | | | | | | DV | | | mag.org/content/early/ |
| | ADYYCYSTDSTANYKVFGGGTKVTVL | | | | | | | | | 2020/06/15/science.abc |
| | | | | | | | | | | 7424) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| mAb-67 | Ab | SARS-CoV2 | 229E, | | | S; Unk | B-cells; SARS-CoV1 | 935 | EVQLVESGPGLVKPSGTLSLTCAVTGAS | 2002 |
| | | | HKU1, | | | | Human Patient | | VSSDHWWSWVRQSPGKGLEWIGEVY | |
| | | | NL63, | | | | | | HSGSTNYNPSLKSRVTISLDQSNNQFSLK | |
| | | | OC43 | | | | | | LTSVTAADTAIYYCATMWGGLCTASNCY | |
| | | | SARS- | | | | | | GNPMDVWGQGTTVTVSS | |
| | | | CoV1 | | | | | | | |
| mAb-68 | Ab | SARS-CoV1, | 229E, | | | S; Unk | B-cells; SARS-CoV1 | 936 | QVQLQESGPGLVKPSGTLSLTCAVSGAS | 2003 |
| | | SARS-CoV2 | HKU1, | | | | Human Patient | | VSSDHWWSWVRQSPGKGLEWIGEVY | |
| | | | NL63, | | | | | | HSGSTNYNPSLKSRVTISLDQSNNQFSLK | |
| | | | OC43 | | | | | | LTSVTAADTAIYYCATMWGGLCTASNCY | |
| | | | | | | | | | GNPMDVWGQGTTVTVSS | |
| mAb-69 | Ab | SARS-CoV1, | 229E, | | | S; Unk | B-cells; SARS-CoV1 | 937 | QVQLQESGGGLVQPGGSLRLSCEASGF | 2004 |
| | | SARS-CoV2 | HKU1, | | | | Human Patient | | SFSDFVMHWVRQVPGKGLEWVSRISH | |
| | | | NL63, | | | | | | DGSITSYVDSVKGRFTVSRDNGKKTLYL | |
| | | | OC43 | | | | | | QMNSPRTEDTAVYYCARDLAWTFFDY | |
| | | | | | | | | | WGHGTLVTVSS | |
| mAb-7 | Ab | SARS-Cov1, | 229E, | | | S; Unk | B-cells; SARS-CoV1 | 938 | EVQLLESGGGLSRPGGSLRLSCAASGFIA | 2005 |
| | | SARS-CoV2 | HKU1, | | | | Human Patient | | SRNCMQWVRQAPGKGLEWVSIICGDEI | |
| | | | NL63, | | | | | | TYIRDSVKGRFTISRDDSKNTLHLEMNSL | |
| | | | OC43 | | | | | | RADDTAVYYCARATPPGGTTGWPYIDL | |
| | | | | | | | | | WGQGTLVTVSS | |
| mAb-70 | Ab | 229E, HKU1 | OC43 | | | S; Unk | B-cells; SARS-CoV1 | 939 | EVQLLESGGGLVQPGGSLRLSCAASGFP | 2006 |
| | | (weak), | | | | | Human Patient | | FSTDAMNWVRQAPGEGLEWVSTISDT | |
| | | NL63, SARS- | | | | | | | GRDTYYAASVKGRFTISRDNSKNTVYLQ | |
| | | CoV1 | | | | | | | MNSLRAEDTAVYYCANTNFLDYWGQG | |
| | | (weak), | | | | | | | TLVTVSS | |
| | | SARS-CoV2 | | | | | | | | |
| mAb-71 | Ab | SARS-CoV2 | 229E, | | | S; Unk | B-cells; SARS-CoV1 | 940 | EVQLVESGGGLVQPGRSLRLSCAASGFIL | 2007 |
| | | | HKU1, | | | | Human Patient | | DDYAVHWVRLAPGKGLEWVSGITWNS | |
| | | | NL63, | | | | | | GYLGYADSVKGRFTISRDNAKNSLYLQM | |
| | | | OC43 | | | | | | NSLRPEDTALYYCAKLGTDHPIGVDVW | |
| | | | SARS- | | | | | | GQGTTVTVSS | |
| | | | CoV1 | | | | | | | |
| mAb-72 | Ab | SARS-CoV2 | 229E, | | | S; Unk | B-cells; SARS-CoV1 | 941 | QVQLVQSGGGLVQPGGSLRLSCSASGF | 2008 |
| | | | HKU1, | | | | Human Patient | | TFNTYTMHWVRQAPGKGLEYVSAISSN | |
| | | | NL63, | | | | | | GVVTYYADSVKGRFTISRDNSKNTLYLQ | |
| | | | OC43 | | | | | | MSSLRAEDTAVYYCVKALYSSSWCPFDY | |
| | | | SARS- | | | | | | WGQGALVTVSS | |
| | | | CoV1 | | | | | | | |
| mAb-73 | Ab | SARS-CoV2 | 229E, | | | S; Unk | B-cells; SARS-CoV1 | 942 | QVQLQQSGGGLVQPGGSLRLSCEASGF | 2009 |
| | | | HKU1, | | | | Human Patient | | NFNSYSMSWVRQAPGKGLEWLSYISSR | |
| | | | NL63, | | | | | | SSTIKYASSVQGRFTVSRDNAKKSVFLQ | |
| | | | OC43 | | | | | | MNSLRDEDTAVYYCARELDSETYYNYNS | |
| | | | SARS- | | | | | | LDVWGQGTTVTVSS | |
| | | | CoV1 | | | | | | | |
| mAb-74 | Ab | SARS-CoV2 | 229E, | | | S; Unk | B-cells; SARS-CoV1 | 943 | QVQLQESGPGLVRPSETLSLTCTVSRGSI | 2010 |
| | | | HKU1, | | | | Human Patient | | SSSYWSWIRQPPGKGLEWIGFMYYSGS | |
| | | | NL63, | | | | | | TNYNPSLKSRVTISLDTSKNQFSLKLSSVT | |
| | | | OC43 | | | | | | AADTAVYYCAKAQGIYYRGWSYWFDP | |
| | | | SARS- | | | | | | WGQGTLVTVSS | |
| | | | CoV1 | | | | | | | |
| mAb-75 | Ab | 229E, HKU1 | OC43 | | | S; Unk | B-cells; SARS-CoV1 | 944 | EVQLVQSGAEVKKPGASVKISCKASGYA | 2011 |
| | | (weak), | | | | | Human Patient | | FRNNYINWIRQAPGQGLEWMGIINPSA | |
| | | NL63, SARS- | | | | | | | GTSTYAQKFQGRVTMTRDTSTNTVYME | |
| | | CoV1 | | | | | | | ITSLRSEDTATYFCAREARRQVTQWFGE | |
| | | (weak), | | | | | | | FWGPYNWFDPWGQGTLVTVSS | |
| | | SARS-CoV2 | | | | | | | | |
| mAb-76 | Ab | SARS-CoV1, | 229E, | | | S; Unk | B-cells; SARS-CoV1 | 945 | QVQLQQWGAGLLKPSETLSLTCGVYGV | 2012 |
| | | SARS-CoV2 | HKU1, | | | | Human Patient | | SFSDYYWSWIRQPPGKGLEWIGEINHS | |
| | | | NL63, | | | | | | GITNYNPSLKSRVTISVDTSKNQFSLKLSS | |
| | | | OC43 | | | | | | VTAADTAVYYCARGLISYTLWLRESYFDY | |
| | | | | | | | | | WGQGTLVTVSS | |
| mAb-77 | Ab | SARS-CoV1, | 229E, | | | S; Unk | B-cells; SARS-CoV1 | 946 | QVQLVQSGAEVKKPGSSVKVSCKASGG | 2013 |
| | | SARS-CoV2 | HKU1, | | | | Human Patient | | SLRDYAISWVRQAPGQGPEWMGGIMP | |
| | | | NL63, | | | | | | IFGTAGYAQKFQGRVKFTADESATTAYM | |
| | | | OC43 | | | | | | ELTGLRSEDSAVYFCARDPSILNTGNHH | |
| | | | | | | | | | WYDLDIWGQGTTVTVSS | |
| mAb-78 | Ab | SARS-CoV2 | 229E, | | | S; Unk | B-cells; SARS-CoV1 | 947 | QVQLVQSGAEVKKPGASVKVSCKASGY | 2014 |
| | | | HKU1, | | | | Human Patient | | TFTNDGFVWVRQAPGQVPEWMGWIS | |
| | | | NL63, | | | | | | VHTGDTIYAQRFQGRVTMTTDTSTRTSY | |
| | | | OC43 | | | | | | MELMSLRSDDTAVYYCARDYGDGPPD | |
| | | | SARS- | | | | | | HWGQGTLVTVSS | |
| | | | CoV1 | | | | | | | |
| mAb-79 | Ab | SARS-CoV1, | 229E, | | | S; Unk | B-cells; SARS-CoV1 | 948 | QVQLVQSGPEVKKPGSSVRVSCKVSGG | 2015 |
| | | SARS-CoV2 | HKU1, | | | | Human Patient | | PFSSYGVSWARQAPEKGLEWMGGVLPI | |
| | | | NL63, | | | | | | FGTVGYVHKFQGRVTITADESTSTVYMA | |
| | | | OC43 | | | | | | LSSLRSEDTAVYYCVLDTTMSHPHNWY | |
| | | | | | | | | | GMDVWGHGTTVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| mAb-67 | SYELTQPPSVSVSPGQTARITCSGDALP | IGHV4-4 | IGHJ6 | IGLV3-10 | IGLJ3 | 3041 | ATMWGGLCT | 4231 | YSTDSTAN | Anna Wec et al., 2020 |
| | RRYAYWYQQRSGQAPVLVIYEDNKRP | (Human) | (Human) | (Human) | (Human) | | ASNCYGNPM | | YKV | (https://science.science |
| | SGIPERFSAFSSGTRATLTISGAQVEDE | | | | | | DV | | | mag.org/content/early/ |
| | ADYYCYSTDSTANYKVFGGGTKLTVL | | | | | | | | | 2020/06/15/science.abc |
| | | | | | | | | | | 7424) |
| mAb-68 | SYELTQPPSVSVSPGQTARITCSGDALP | IGHV4-4 | IGHJ6 | IGLV3-10 | IGLJ3 | 3042 | ATMWGGLCT | 4232 | YSTDSTAN | Anna Wec et al., 2020 |
| | RRYAYWYQQRSGQAPVLVIYEDNKRP | (Human) | (Human) | (Human) | (Human) | | ASNCYGNPM | | YKV | (https://science.science |
| | SGIPERFSAFSSGTRATLTISGAQVEDE | | | | | | DV | | | mag.org/content/early/ |
| | ADYYCYSTDSTANYKVFGGGTKLTVL | | | | | | | | | 2020/06/15/science.abc |
| | | | | | | | | | | 7424) |
| mAb-69 | QSVLTQPSSLSASPGASASLTCTLHSGF | IGHV3-74 | IGHJ4 | IGLV5-45 | IGLJ3 | 3043 | ARDLAWTFF | 4233 | MIWHDNA | Anna Wec et al., 2020 |
| | NVGDHTINWYQQRPGSPPRYLLKYKS | (Human) | (Human) | (Human) | (Human) | | DY | | VV | (https://science.science |
| | DSDKEQGSGVPIRFSGSKDASANAGFL | | | | | | | | | mag.org/content/early/ |
| | LISGLRSEDEADYYCMIWHDNAVVFG | | | | | | | | | 2020/06/15/science.abc |
| | GGTKLTVL | | | | | | | | | 7424) |
| mAb-7 | EIVLTQSPATLSVSPGDGASLSCRASQS | IGHV3-66 | IGHJ4 | IGKV3-15 | IGKJ4 | 3044 | ARATPPGGTT | 4234 | HQYNTWP | Anna Wec et al., 2020 |
| | VGSNLAWYQQKPGQAPRLLISDASAR | (Human) | (Human) | (Human) | (Human) | | GWPYIDL | | PLT | (https://science.science |
| | ATGVPARFTGSGSGTDFTLTISSLQSED | | | | | | | | | mag.org/content/early/ |
| | FAVYYCHQYNTWPPLTFGGGTKLEIK | | | | | | | | | 2020/06/15/science.abc |
| | | | | | | | | | | 7424) |
| mAb-70 | EIVMTQSPLSLPVTLGQSASISCRSSQG | IGHV3-23 | IGHJ4 | IGKV2-30 | IGKJ3 | 3045 | ANTNFLDY | 4235 | MQATHW | Anna Wec et al., 2020 |
| | LVHSDGNIYLNWFQQRPGQSPRRLIY | (Human) | (Human) | (Human) | (Human) | | | | PRA | (https://science.science |
| | KVSNRDSGVPDRFSGSGSGTDFTLKIS | | | | | | | | | mag.org/content/early/ |
| | RVEAEDVGLYYCMQATHWPRAFGQ | | | | | | | | | 2020/06/15/science.abc |
| | GTKVDIK | | | | | | | | | 7424) |
| mAb-71 | DIVMTQSPLALSVTPGQPASISCKSSQ | IGHV3-9 | IGHJ6 | IGKV2D-29 | IGKJ5 | 3046 | AKLGTDHPIG | 4236 | MQSIQVPI | Anna Wec et al., 2020 |
| | SLLHSDGKTYFYWYLQKPGQSPHLLIY | (Human) | (Human) | (Human) | (Human) | | VDV | | T | (https://science.science |
| | EVSNRFSGVPDRFSGSGSGTDFTLKISR | | | | | | | | | mag.org/content/early/ |
| | VEAEDVGVYYCMQSIQVPITFGGGTK | | | | | | | | | 2020/06/15/science.abc |
| | LEIK | | | | | | | | | 7424) |
| mAb-72 | DIVMTQSPATLSVSPGERATLSCRASQ | IGHV3- | IGHJ4 | IGKV3-15 | IGKJ3 | 3047 | VKALYSSSWC | 4237 | QQYNLWP | Anna Wec et al., 2020 |
| | SVSSNLAWYQQKPGQAPRLLIYGAST | 64D | (Human) | (Human) | (Human) | | PFDY | | YT | (https://science.science |
| | RATNIPARFSGSGSGTEFTLTISSLQSE | (Human) | | | | | | | | mag.org/content/early/ |
| | DFAVYYCQQYNLWPYTFGQGTKVDIK | | | | | | | | | 2020/06/15/science.abc |
| | | | | | | | | | | 7424) |
| mAb-73 | EIVLTQSPSSLSASVGDRVTISCRASQGI | IGHV3-48 | IGHJ6 | IGKV1-16 | IGKJ2 | 3048 | ARELDSETYY | 4238 | KQYNSYPY | Anna Wec et al., 2020 |
| | STFLAWFQQRPGKAPKSLIYAASKLQS | (Human) | (Human) | (Human) | (Human) | | NYNSLDV | | T | (https://science.science |
| | GVPSRFSGSDSGPDFTLTIDNLQPEDS | | | | | | | | | mag.org/content/early/ |
| | ATYYCKQYNSYPYTFGQGTKLEIK | | | | | | | | | 2020/06/15/science.abc |
| | | | | | | | | | | 7424) |
| mAb-74 | QSVVTQPPSVSAAPGQKVTISCSGSSS | IGHV4-59 | IGHJ5 | IGLV1-51 | IGLJ3 | 3049 | AKAQGIYYRG | 4239 | ETWDDSLS | Anna Wec et al., 2020 |
| | NIGNNYVSWYQQLPGTAPKLLIYDSD | (Human) | (Human) | (Human) | (Human) | | WSYWFDP | | AVV | (https://science.science |
| | KRPSGIPERFSGSKSATSATLGITGLQT | | | | | | | | | mag.org/content/early/ |
| | GDEADYYCETWDDSLSAVVFGGGTKL | | | | | | | | | 2020/06/15/science.abc |
| | TVL | | | | | | | | | 7424) |
| mAb-75 | DIVLTQSPLSLSVTPGQPASISCRSSQSL | IGHV1-46 | IGHJ5 | IGKV2D-29 | IGKJ5 | 3050 | AREARRQVT | 4240 | MQSIQTPI | Anna Wec et al., 2020 |
| | QHTDGKTYLYWYLQKPGQSPQLLIYEL | (Human) | (Human) | (Human) | (Human) | | QWFGEFWG | | T | (https://science.science |
| | FNRFSGVPERFSGSGSGTDFTLKISRVE | | | | | | PYNWFDP | | | mag.org/content/early/ |
| | AEDVGIYYCMQSIQTPITFGQGTRLEIK | | | | | | | | | 2020/06/15/science.abc |
| | | | | | | | | | | 7424) |
| mAb-76 | DIRVTQSPDSLAVSLGERATINCKSSQS | IGHV4-34 | IGHJ4 | IGKV4-1 | IGKJ4 | 3051 | ARGLISYTLW | 4241 | QQYYSTPP | Anna Wec et al., 2020 |
| | VLYSSNNKNYLAWYQQKIGQPPKLLIY | (Human) | (Human) | (Human) | (Human) | | LRESYFDY | | T | (https://science.science |
| | WASIRESGVPDRFTGSGSGTDFTLTISS | | | | | | | | | mag.org/content/early/ |
| | LQAEDVAVYYCQQYYSTPPTFGGGTK | | | | | | | | | 2020/06/15/science.abc |
| | LEIK | | | | | | | | | 7424) |
| mAb-77 | EIVMTQTPLSLPVTLGQPASISCRSSQF | IGHV1-69 | IGHJ6 | IGKV2-30 | IGKJ1 | 3052 | ARDPSILNTG | 4242 | MQATEWP | Anna Wec et al., 2020 |
| | VSHTDGNTYLNWFQQRPGQSPRRLIY | (Human) | (Human) | (Human) | (Human) | | NHHWYDLDI | | RT | (https://science.science |
| | KVSKRDSGVPDRFSGSGSGTDFTLTIS | | | | | | | | | mag.org/content/early/ |
| | RVEAEDVGVYYCMQATEWPRTFGQG | | | | | | | | | 2020/06/15/science.abc |
| | TKVEYQ | | | | | | | | | 7424) |
| mAb-78 | DIVMTQSPDSLAVSLGERATINCKSSQ | IGHV1-18 | IGHJ4 | IGKV4-1 | IGKJ3 | 3053 | ARDYGDGPP | 4243 | QQYYTTPL | Anna Wec et al., 2020 |
| | SVLFSSNNKNYLAWYQQKPGLPPKLLI | (Human) | (Human) | (Human) | (Human) | | DH | | FT | (https://science.science |
| | YWASTRKSGVSDRFSGSGSGTDFTLTI | | | | | | | | | mag.org/content/early/ |
| | SSLQAEDVAVYYCQQYYTTPLFTFGPG | | | | | | | | | 2020/06/15/science.abc |
| | TKVDIK | | | | | | | | | 7424) |
| mAb-79 | DIVMTQSPLSLPVTLGQPASISCRSSHS | IGHV1-69 | IGHJ6 | IGKV2-30 | IGKJ3 | 3054 | VLDTTMSHP | 4244 | MQGTDW | Anna Wec et al., 2020 |
| | LVHTNGNTYLNWFQQRPGQPPRRLIY | (Human) | (Human) | (Human) | (Human) | | HNWYGMDV | | PRT | (https://science.science |
| | QVSNRDSGVPDRFSGSGSGTDFTLKIS | | | | | | | | | mag.org/content/early/ |
| | RVEADDVGIYYCMQGTDWPRTFGQG | | | | | | | | | 2020/06/15/science.abc |
| | TKVDIK | | | | | | | | | 7424) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| mAb-8 | Ab | SARS-CoV1, | 229E, | | | S; Unk | B-cells; SARS-CoV1 | 949 | QVQLVQSGAEVKKPGSSMKLSCKASGI | 2016 |
| | | SARS-CoV2 | HKU1, | | | | Human Patient | | NFRSYSFSWVRQAPGQGLEWMGGVIP | |
| | | | NL63, | | | | | | YFPTANYAEKFRGRVTATADESTGTVYLE | |
| | | | OC43 | | | | | | MSSLRSEDTAVYYCASEYFDGRSYHSFC | |
| | | | | | | | | | GLDVWGQGTLVTVSS | |
| mAb-80 | Ab | 229E, HKU1 | OC43 | | | S; Unk | B-cells; SARS-CoV1 | 950 | QVQLVQSGAEVKKPGSSVKVSCKASGG | 2017 |
| | | (weak), | | | | | Human Patient | | TLSDYAISWVRQAPGQGLEWMGGIMP | |
| | | NL63, SARS- | | | | | | | VFGSPGYAEIFQGRLTITADESRSTAYME | |
| | | CoV1 | | | | | | | LTSLRSEDTAVYYCARDPSILNTGPHHW | |
| | | (weak), | | | | | | | YDLDIWGPGTTVTVSS | |
| | | SARS-CoV2 | | | | | | | | |
| mAb-81 | Ab | SARS-CoV2 | 229E, | | | S; Unk | B-cells; SARS-CoV1 | 951 | EVQLVQSGAEVKKPGSSMRVSCRVSGG | 2018 |
| | | | HKU1, | | | | Human Patient | | TFITHAMSWVRQAPGQGPEWMGGIV | |
| | | | NL63, | | | | | | PLFGRASYAQPSQTRVQITADESTSTVYL | |
| | | | OC43, | | | | | | EVPSLTSEDTAVYYCVRDSEPYTATRSQN | |
| | | | SARS- | | | | | | HYWYDMDVWGQGTTVTVSS | |
| | | | CoV1 | | | | | | | |
| mAb-82 | Ab | HKU1, SARS- | 229E, | | | S; Unk | B-cells; SARS-CoV1 | 952 | EVQLVESGPGLVKPSATLSLTCTVSGGSI | 2019 |
| | | CoV1, SARS- | NL63, | | | | Human Patient | | NNYYWTWVRQSAGKGLEWIGRINTSG | |
| | | CoV2 | OC43 | | | | | | STNYNASLKSRVTMSIDTSKNEFSLRLSS | |
| | | | | | | | | | VTAADTAVYYCAREFGVRFLDRSLFGAM | |
| | | | | | | | | | DVWGHGATVTVSS | |
| mAb-83 | Ab | SARS-CoV2 | 229E, | | | S; Unk | B-cells; SARS-CoV1 | 953 | QVQLVESGGGVVQPGRSLRLSCAASGF | 2020 |
| | | | HKU1, | | | | Human Patient | | TFSAFAMHWVRQAPGKGLEWVTIISYD | |
| | | | NL63, | | | | | | GSNEYYADSVQGRFSISRDNSKNTLFLQ | |
| | | | OC43, | | | | | | MSSLRTEDTAIYYCARAGGYLSAFDIWG | |
| | | | SARS- | | | | | | QGTTVTVSS | |
| | | | CoV1 | | | | | | | |
| mAb-84 | Ab | SARS-CoV1, | 229E, | | | S; Unk | B-cells; SARS-CoV1 | 954 | EVQLVQSGAEVKKPGSSVKVSCKVSGGT | 2021 |
| | | SARS-CoV2 | HKU1, | | | | Human Patient | | FSNYAISWLRQAPGQGPEWMGGIIPAL | |
| | | | NL63, | | | | | | SKVGYAGKFQARLTFSADELKTTVYMDL | |
| | | | OC43 | | | | | | SSLTSEDTAVYYCARDPSFLNAGNHFYYD | |
| | | | | | | | | | FDVWGQGTMVTVSS | |
| mAb-85 | Ab | SARS-CoV2 | 229E, | | | S; Unk | B-cells; SARS-CoV1 | 955 | QVQLQESGPGLVKPSGTLSLTCAVSGGS | 2022 |
| | | | HKU1, | | | | Human Patient | | ITSRDWWSWVRQPPGKGLEWIGEVYH | |
| | | | NL63, | | | | | | SGSTSYNPSLKSRVTISVDKSKNEFSLKLS | |
| | | | OC43, | | | | | | SVTAADTAVYYCARAGNIVVMPAAQYY | |
| | | | SARS- | | | | | | FDYWGQGTLVTVSS | |
| | | | CoV1 | | | | | | | |
| mAb-86 | Ab | SARS-CoV1, | 229E, | | | S; Unk | B-cells; SARS-CoV1 | 956 | EVQLVESGGSVVQPGTSLKLSCAGSAGL | 2023 |
| | | SARS-CoV2 | HKU1, | | | | Human Patient | | TITRYAMHWVRQAPGKGLEWVALVSH | |
| | | | NL63, | | | | | | DGIHIGYSDSVRGRFTISRDNSRNTLYLQ | |
| | | | OC43 | | | | | | MDGLRPEDTAVYYCVRDDVLQHSRPSG | |
| | | | | | | | | | PGYFVSWGQGTLVTVSS | |
| mAb-87 | Ab | SARS-CoV2 | 229E, | | | S; Unk | B-cells; SARS-CoV1 | 957 | QVQLQESGPGLVKPSETLSLTCTVSGGS | 2024 |
| | | | HKU1, | | | | Human Patient | | MNHYYWSWIRQPPGKGLEGIGYTYYSG | |
| | | | NL63, | | | | | | STNYNPSLKSRVTISVDASKNQFSLRLSSV | |
| | | | OC43, | | | | | | TAADTAVYYCARGSQIDLRGGLGATFFD | |
| | | | SARS- | | | | | | YWGQGTLVTVSS | |
| | | | CoV1 | | | | | | | |
| mAb-88 | Ab | SARS-CoV2 | 229E, | | | S; Unk | B-cells; SARS-CoV1 | 958 | QVQLVESGAEVKKPGSSVKISCKISGDTF | 2025 |
| | | | HKU1, | | | | Human Patient | | STNAISWLRQAPGREPEWMGGIVPLVG | |
| | | | NL63, | | | | | | PASYAQRPQGRLTITADEFTNTAYLELNS | |
| | | | OC43, | | | | | | LRSEDTATYYCARDSDPYTATRRHNHYW | |
| | | | SARS- | | | | | | YAMDVWGQGTTVTVSS | |
| | | | CoV1 | | | | | | | |
| mAb-89 | Ab | SARS-CoV1, | 229E, | | | S; Unk | B-cells; SARS-CoV1 | 959 | EVQLLESGGGLVQPGGSLRLSCAASGFIF | 2026 |
| | | SARS-CoV2 | HKU1, | | | | Human Patient | | KNEPMNWIRQFPGKGLEWISNIRDNG | |
| | | | NL63, | | | | | | NDVYYADSVKGRFTVSRDNAKNSLYLQ | |
| | | | OC43 | | | | | | MNSLRDDDTALYYCVRDTDWAFDSWG | |
| | | | | | | | | | QGTLVTVSS | |
| mAb-9 | Ab | SARS-CoV1, | 229E, | | | S; Unk | B-cells; SARS-CoV1 | 960 | QVQLVQSGAEVKKPGASVKVSCKASGY | 2027 |
| | | SARS-CoV2 | HKU1, | | | | Human Patient | | TFSSYDINWVRQATGQGLEWMGWMS | |
| | | | NL63, | | | | | | PNTGDTGYAHKFQGRVRMTSNTSISTA | |
| | | | OC43 | | | | | | YMELNSLTSEDTAVYYCARRGNNFGYYY | |
| | | | | | | | | | YYTVDVWGQGTTVTVSS | |
| mAb-90 | Ab | HKU1, SARS- | 229E, | | | S; Unk | B-cells; SARS-CoV1 | 961 | QVQLVQSGADVKKPGASVKVSCKASGY | 2028 |
| | | CoV1, SARS- | NL63, | | | | Human Patient | | TFTSYYMHWVRQAPGQGLEWLGVIHP | |
| | | CoV2 | OC43 | | | | | | SGGSTTFAQKFQGRVTMTRDTSTSTVY | |
| | | | | | | | | | MELSSLRSEDTAVYYCARVLAGSSHEW | |
| | | | | | | | | | QLTHDAFDIWGQGTTVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| mAb-8 | ETTLTQSPLSLPVTLGQPASISCRSSQG | IGHV1-69 | IGHJ4 | IGKV2-30 | IGKJ1 | 3055 | ASEYFDGRSY | 4245 | MQGTEW | Anna Wec et al., 2020 |
| | LAHSNGNTYLNWFHQRPGQSPRRLIY | (Human) | (Human) | (Human) | (Human) | | HSFCGLDV | | PRT | (https://science.science |
| | QVSNRDSGVPDRFSGSGSGTDFTLKIS | | | | | | | | | mag.org/content/early/ |
| | RVEAEDVGVYYCMQGTEWPRTFGQ | | | | | | | | | 2020/06/15/science.abc |
| | GTKVEIK | | | | | | | | | 7424) |
| mAb-80 | DIVMITQSPLSLPVSLGQPASISCRSSQS | IGHV1-69 | IGHJ6 | IGKV2-30 | IGKJ3 | 3056 | ARDPSILNTG | 4246 | MQGTEW | Anna Wec et al., 2020 |
| | VVHTDGNTYLNWYQQRPGQSPRRLIY | (Human) | (Human) | (Human) | (Human) | | PHHWYDLDI | | PRT | (https://science.science |
| | KVSNRDSGVPDRFSGSGSVTDFTLKIS | | | | | | | | | mag.org/content/early/ |
| | RVEAEDVAVYYCMQGTEWPRTFGQG | | | | | | | | | 2020/06/15/science.abc |
| | TKVDIK | | | | | | | | | 7424) |
| mAb-81 | DIQMTQSPLSLSVTLGQSASISCRASQT | IGHV1-69 | IGHJ6 | IGKV2-30 | IGKJ1 | 3057 | VRDSEPYTAT | 4247 | MQGTDW | Anna Wec et al., 2020 |
| | VVHSVDGNTYLNWFHQRPGQSPRRLI | (Human) | (Human) | (Human) | (Human) | | RSQNHYWYD | | PRT | (https://science.science |
| | YKVSNRDSGVPDRFSGSGSGTDFTLRI | | | | | | MDV | | | mag.org/content/early/ |
| | SRVEAEDIGIYYCMQGTDWPRTFGQG | | | | | | | | | 2020/06/15/science.abc |
| | TKVEIK | | | | | | | | | 7424) |
| mAb-82 | QPVLTQPASVSGSPGQSITISCTGTSSD | IGHV4-4 | IGHJ6 | IGLV2-14 | IGLJ1 | 3058 | AREFGVRFLD | 4248 | SSYTSSSTL | Anna Wec et al., 2020 |
| | VGSFNYVSWYQQHPGKAPKLVIYDVY | (Human) | (Human) | (Human) | (Human) | | RSLFGAMDV | | YV | (https://science.science |
| | NRPSGVSNRFSGSKSGNTASLTISGLQ | | | | | | | | | mag.org/content/early/ |
| | AEDEADYYCSSYTSSSTLYVFGTGTKVT | | | | | | | | | 2020/06/15/science.abc |
| | VL | | | | | | | | | 7424) |
| mAb-83 | DIRLTQSPSTLSASVGDRVTITCRASQSI | IGHV3-30 | IGHJ3 | IGKV1-5 | IGKJ1 | 3059 | ARAGGYLSAF | 4249 | QQYNSGW | Anna Wec et al., 2020 |
| | SSWLAWYQQKPGKAPNLLIYKASTLES | (Human) | (Human) | (Human) | (Human) | | DI | | T | (https://science.science |
| | GVPSRFSGSGSGTEFTLTISSLQPDDFA | | | | | | | | | mag.org/content/early/ |
| | TYYCQQYNSGWTFGQGTKVEIK | | | | | | | | | 2020/06/15/science.abc |
| | | | | | | | | | | 7424) |
| mAb-84 | DIRLTQSPLSLPVTLGQPASISCTSSQD | IGHV1-69 | IGHJ3 | IGKV2-30 | IGKJ3 | 3060 | ARDPSFLNAG | 4250 | MQGTDW | Anna Wec et al., 2020 |
| | VVHTDGNTYLNWFHQRPGQSPRRLIY | (Human) | (Human) | (Human) | (Human) | | NHFYYDFDV | | PRT | (https://science.science |
| | KVSNRDSGVPDRFSGSGSGSHFTLKIS | | | | | | | | | mag.org/content/early/ |
| | RVEAEDAGIYYCMQGTDWPRTFGQG | | | | | | | | | 2020/06/15/science.abc |
| | TKVDIK | | | | | | | | | 7424) |
| mAb-85 | EIVLTQSPSSLSASVGDRVTITCRASQDI | IGHV4-4 | IGHJ4 | IGKV1-9 | IGKJ3 | 3061 | ARAGNIVVM | 4251 | LQLHNYSS | Anna Wec et al., 2020 |
| | SNYLAWYQQKPGKAPKLLIYAASTLQS | (Human) | (Human) | (Human) | (Human) | | PAAQYYFDY | | | (https://science.science |
| | GVPLRFSGSGSGTDFTLTISSLQPEDFA | | | | | | | | | mag.org/content/early/ |
| | TYYCLQLHNYSSFGGGTKVDIK | | | | | | | | | 2020/06/15/science.abc |
| | | | | | | | | | | 7424) |
| mAb-86 | EIVLTQSPATLSLSPGERATLSCRASQS | IGHV3-30 | IGHJ5 | IGKV3-11 | IGKJ2 | 3062 | VRDDVLQHS | 4252 | QQRSDGY | Anna Wec et al., 2020 |
| | VSTFLGWYQQRPGQPPRLLIYDASYR | (Human) | (Human) | (Human) | (Human) | | RPSGPGYFVS | | N | (https://science.science |
| | APDIPVRFSGSGSGTDFTLTINSLEPED | | | | | | | | | mag.org/content/early/ |
| | SAVYYCQQRSDGYNFGPGTKLEIK | | | | | | | | | 2020/06/15/science.abc |
| | | | | | | | | | | 7424) |
| mAb-87 | QSVLTQPASVSGSPGQSITISCTGTSSD | IGHV4-59 | IGHJ4 | IGLV2-14 | IGLJ3 | 3063 | ARGSQIDLRG | 4253 | SSYTSTNTV | Anna Wec et al., 2020 |
| | VGAYNYVSWYQQHPGKAPKLTVYDV | (Human) | (Human) | (Human) | (Human) | | GLGATFFDY | | | (https://science.science |
| | RNRPSGVSNRFSGSKSGNTASLTISGL | | | | | | | | | mag.org/content/early/ |
| | QAEDEAHYYCSSYTSTNTVFGGGTKLT | | | | | | | | | 2020/06/15/science.abc |
| | VL | | | | | | | | | 7424) |
| mAb-88 | DIRLTQTPLSLPVTLGQPASISCRSSQIA | IGHV1-69 | IGHJ6 | IGKV2-24 | IGKJ3 | 3064 | ARDSDPYTAT | 4254 | MQGTDW | Anna Wec et al., 2020 |
| | MHSDGNTYLSWFHQRPGQPPRRLIYK | (Human) | (Human) | (Human) | (Human) | | RRHNHYWYA | | PRT | (https://science.science |
| | ISNRDSGVPDRFSGSGSGTDFTLKISRV | | | | | | MDV | | | mag.org/content/early/ |
| | EAEDIGTFYCMQGTDWPRTFGQGTK | | | | | | | | | 2020/06/15/science.abc |
| | VDIK | | | | | | | | | 7424) |
| mAb-89 | QSALIQPRSVSGSPGQSVTISCTGSSST | IGHV3-48 | IGHJ5 | IGLV2-11 | IGLJ3 | 3065 | VRDTDWAFD | 4255 | CSSPGTIT | Anna Wec et al., 2020 |
| | FGTDNHISWYQQLPGKVPKLIIHAVSQ | (Human) | (Human) | (Human) | (Human) | | S | | | (https://science.science |
| | RPSVVPDRFSGSRSGNTASLTISGLQA | | | | | | | | | mag.org/content/early/ |
| | EDEADYYCCSSPGTITWVFGGGTKVT | | | | | | | | | 2020/06/15/science.abc |
| | VL | | | | | | | | | 7424) |
| mAb-9 | DIRLTQSPSSLSASVGDRVTIACRASQS | IGHV1-8 | IGHJ6 | IGKV1-39 | IGKJ1 | 3066 | ARRGNNFGY | 4256 | QQCYSYPP | Anna Wec et al., 2020 |
| | VSNYLNWYQQKPGKAPKLLIYAASSLQ | (Human) | (Human) | (Human) | (Human) | | YYYYTVDV | | T | (https://science.science |
| | NGVPSRFGGSGSGTDFTLTISSLQPED | | | | | | | | | mag.org/content/early/ |
| | FATYYCQQCYSYPPTFGHGTKVEIK | | | | | | | | | 2020/06/15/science.abc |
| | | | | | | | | | | 7424) |
| mAb-90 | DIRMTQSPDSLAVSLGERATINCRTSQ | IGHV1-46 | IGHJ3 | IGKV4-1 | IGKJ3 | 3067 | ARVLAGSSHE | 4257 | QQYYSTPY | Anna Wec et al., 2020 |
| | SVLYSSNNKNYLGWYQQKPGQPPKLL | (Human) | (Human) | (Human) | (Human) | | WQLTHDAFD | | T | (https://science.science |
| | IYWASTRESGVPDRFSGSGSGTDFTLTI | | | | | | I | | | mag.org/content/early/ |
| | SSLQAEDVAVYYCQQYYSTPYTFGQGT | | | | | | | | | 2020/06/15/science.abc |
| | KVDIK | | | | | | | | | 7424) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| mAb-91 | Ab | HKU1 | 229E, | | | S; Unk | B-cells; SARS-CoV1 | 962 | EVQLLESGGGLVQPGGSLRLSCAASGFT | 2029 |
| | | (weak), | NL63, | | | | Human Patient | | FSSYAMSWVRQAPGKGLEWVSGIDGG | |
| | | SARS-CoV1 | OC43 | | | | | | GGSSYYADSVRGRFTVSRDNSKNMLHL | |
| | | (weak), | | | | | | | QMNSLRADDTAVYFCAKGDWIRYFDW | |
| | | SARS-CoV2 | | | | | | | SLPISFFDYWGQGALVTVSS | |
| mAb-92 | Ab | SARS-CoV1, | 229E, | | | S; Unk | B-cells; SARS-CoV1 | 963 | EVQLLESGPGLLKPSETLSLTCTVSGDSV | 2030 |
| | | SARS-CoV2 | HKU1, | | | | Human Patient | | SSGNFYWSWVRRPPGKALEWIAYSHYT | |
| | | | NL63, | | | | | | GGTNSDPSFMGRVTMSIDPSRNQFSLR | |
| | | | OC43 | | | | | | LTSVAAADTAVYYCARTTSPLTYSGHWP | |
| | | | | | | | | | LFDYWGQGSLVTVSS | |
| mAb-93 | Ab | SARS-CoV1, | 229E, | | | S; Unk | B-cells; SARS-CoV1 | 964 | QVQLVQSGDEMKKPGSSVKVSCKASGD | 2031 |
| | | SARS-CoV2 | HKU1, | | | | Human Patient | | TFSTHAISWVRQAPGQGPEWMGGIIPL | |
| | | | NL63, | | | | | | FGTASYAQTSQSRVKITADESTSTAYMEL | |
| | | | OC43 | | | | | | SSLTSEDTAVYYCVRDSDPYTATSRNNHY | |
| | | | | | | | | | WYGMDVWGQGTTVTVSS | |
| mAb-94 | Ab | SARS-CoV2 | 229E, | | | S; Unk | B-cells; SARS-CoV1 | 965 | QVQLVQSGAEVKKPGSSVKVSCKASGG | 2032 |
| | | | HKU1, | | | | Human Patient | | MFTDYAISWVRQAPGQRLEWMGGIM | |
| | | | NL63, | | | | | | PGLGSPAYAQIFRDRATISADVSTSTAYL | |
| | | | OC43, | | | | | | ELTSLKPEDTAVYYCARDPSILNTGNHH | |
| | | | SARS- | | | | | | WYDLDIWGQGTTVTVSS | |
| | | | CoV1 | | | | | | | |
| mAb-95 | Ab | SARS-CoV1, | 229E, | | | S; Unk | B-cells; SARS-CoV1 | 966 | QVQLVQSGAEVKKPGSSVKVSCKASGG | 2033 |
| | | SARS-CoV2 | HKU1, | | | | Human Patient | | MFTDYAISWVRQAPGQRPEWMGGIM | |
| | | | NL63, | | | | | | PGLGSPAYAQIFRGRATISADLSTSTAYLE | |
| | | | OC43 | | | | | | LTSLKPEDTAVYYCARDPSILNTGNHHW | |
| | | | | | | | | | YDLDIWGQGTMVTVSS | |
| mAb-96 | Ab | SARS-CoV2 | 229E, | | | S; Unk | B-cells; SARS-CoV1 | 967 | QVTLKESGGGLVQPGGSLRLSCAASGFT | 2034 |
| | | | HKU1, | | | | Human Patient | | VNSYGMSWVRQAPGKGLEWVSGFKSR | |
| | | | NL63, | | | | | | SDRPDYAESVKGRFTISRDTSKNTVYLE | |
| | | | OC43, | | | | | | MIGLRAEDTATYYCVRMDWMEWMKY | |
| | | | SARS- | | | | | | YFDSWGQGALVTVSS | |
| | | | CoV1 | | | | | | | |
| mAb-97 | Ab | SARS-CoV1 | 229E, | | | S; Unk | B-cells; SARS-CoV1 | 968 | QVQLVQSGAEVKKPGSSVKVSCKASGG | 2035 |
| | | (weak), | HKU1, | | | | Human Patient | | MISDYAISWVRQAPGQRLEWMGGIMP | |
| | | SARS-CoV2 | NL63, | | | | | | AFGSPGYAQIFRGRATISADVSTNTAYLE | |
| | | | OC43 | | | | | | LTSLNPDDTAVYYCARDPSILNTGNHHW | |
| | | | | | | | | | YDLDMWGQGTMVTVSS | |
| mAb-98 | Ab | SARS-CoV1, | 229E, | | | S; Unk | B-cells; SARS-CoV1 | 969 | QVQLVESGGSVVQPGTSLKLSCAGSAGL | 2036 |
| | | SARS-CoV2 | HKU1, | | | | Human Patient | | TITRYAMHWVRQAPGKGLEWVALVSH | |
| | | (weak) | NL63, | | | | | | DGIHIGYSDSVRGRFTISRDNSRNTLYLQ | |
| | | | OC43 | | | | | | MDGLRPEDTAVYYCVRDDVLQHSRPSG | |
| | | | | | | | | | PGYFVSWGQGTLVTVSS | |
| mAb-99 | Ab | SARS-CoV2 | 229E, | | | S; Unk | B-cells; SARS-CoV1 | 970 | QVQLQESGPGLVKPSGTLSLTCAVSGAS | 2037 |
| | | (weak) | HKU1, | | | | Human Patient | | VSSDHWWSWVRQSPGKGLEWIGEVY | |
| | | | NL63, | | | | | | HSGSTNYNPSLKSRVTISLDQSNNQFSLK | |
| | | | OC43, | | | | | | LTSVTAADTAIYYCATMWGGLCTASNCY | |
| | | | SARS- | | | | | | GNPMDVWGQGTTVTVSS | |
| | | | CoV1 | | | | | | | |
| mBG17 | Ab | SARS-CoV1, | 229E | | | N | Immunised Mouse | 971 | EVKLEESGGGLVQPGGSMKFSCVASGF | 2038 |
| | | SARS-CoV2, | | | | | | | TFSDYWMNWVRQSPDKGLEWVAEIRL | |
| | | NL63 | | | | | | | KSNNYATHYAASVKGRFTISRDDSKSSVY | |
| | | (weak) | | | | | | | LQMNNLRAEDSGIYYCTRSAMDYWGQ | |
| | | | | | | | | | GTSVTVSS | |
| mBG21 | Ab | SARS-CoV1, | 229E | | | N | Immunised Mouse | 972 | QIQLVQSGPELKKPGETVKISCKASGYTF | 2039 |
| | | SARS-CoV2, | | | | | | | TDYSMHWVKQAPGKGSKWMGWINTE | |
| | | NL63 | | | | | | | TGEPTYADDFKGRFAFSLETSASTAYLQI | |
| | | (weak) | | | | | | | NNLKNEDTATYFCALRRWGQGTLVTVS | |
| | | | | | | | | | S | |
| mBG22 | Ab | SARS-CoV1, | 229E | | | N | Immunised Mouse | 973 | QIQLVQSGPELKKPGETVKISCKASGYTF | 2040 |
| | | SARS-CoV2, | | | | | | | TDYSMHWVKQAPGKGSKWMGWINTE | |
| | | NL63 | | | | | | | TGEPTYADDFKGRFAFSLETSASTAYLQI | |
| | | (weak) | | | | | | | NNLKNEDTATYFCALRRWGQGTLVTVS | |
| | | | | | | | | | A | |
| mBG57 | Ab | SARS-CoV1, | 229E | | | N | Immunised Mouse | 974 | QIQLVQSGPELKKPGETVKISCKASGYTF | 2041 |
| | | SARS-CoV2, | | | | | | | TDYSMHWVKQAPGKGSKWMGWINTE | |
| | | NL63 | | | | | | | TGEPTYADDFKGRFAFSLETSASTAYLQI | |
| | | (weak) | | | | | | | NNLKNEDTATYFCALRRWGQGTLVTVS | |
| | | | | | | | | | A | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| mAb-91 | QSALTQPASVSGSPGQSITISCTGTNSD | IGHV3-23 | IGHJ4 | IGLV2-14 | IGLJ3 | 3068 | AKGDWIRYF | 4258 | CSYTRTNT | Anna Wec et al., 2020 |
| | ISNSYSVSWYQQYPGKAPKLVIFDVIN | (Human) | (Human) | (Human) | (Human) | | DWSLPISFFD | | PVL | (https://science.science |
| | RPSGVSNRFSGSKSGNTASLTISGLQA | | | | | | Y | | | mag.org/content/early/ |
| | DDEADYYCCSYTRTNTPVLFGGGTKLT | | | | | | | | | 2020/06/15/science.abc |
| | VL | | | | | | | | | 7424) |
| mAb-92 | DIQLTQSPATLSVSPGERATLSCRASQS | IGHV4-61 | IGHJ4 | IGKV3-15 | IGKJ4 | 3069 | ARTTSPLTYSG | 4259 | QQYYSWP | Anna Wec et al., 2020 |
| | VTKHLAWYQQKPGQAPRLLIYGASTR | (Human) | (Human) | (Human) | (Human) | | HWPLFDY | | PLT | (https://science.science |
| | ATGVPARFSGSGSDTEFSLTISSLQSED | | | | | | | | | mag.org/content/early/ |
| | FAVYYCQQYYSWPPLTFGGGTKLEIK | | | | | | | | | 2020/06/15/science.abc |
| | | | | | | | | | | 7424) |
| mAb-93 | DIVMTQTPLSLPVTLGQPASISCRASQ | IGHV1-69 | IGHJ6 | IGKV2-30 | IGKJ3 | 3070 | VRDSDPYTAT | 4260 | MQGTDW | Anna Wec et al., 2020 |
| | TVVHTNGNTYLNWFHQRPGQSPRRLI | (Human) | (Human) | (Human) | (Human) | | SRNNHYWYG | | PRT | (https://science.science |
| | YEVSNRDSGVPDRFSGSGSGTDFTLSI | | | | | | MDV | | | mag.org/content/early/ |
| | SRVEAEDIGVYYCMQGTDWPRTFGP | | | | | | | | | 2020/06/15/science.abc |
| | GTKVEIK | | | | | | | | | 7424) |
| mAb-94 | DIQLTQSPLSLPVGLGQSASISCRSSQR | IGHV1-69 | IGHJ6 | IGKV2-30 | IGKJ2 | 3071 | ARDPSILNTG | 4261 | MQSTDWP | Anna Wec et al., 2020 |
| | VVHTDGNTYLNWYHQRPGQSPRRLIY | (Human) | (Human) | (Human) | (Human) | | NHHWYDLDI | | RT | (https://science.science |
| | KVSNRDSGVPDRFSGSGSGTDFTLRIS | | | | | | | | | mag.org/content/early/ |
| | RVEAEDVGVYYCMQSTDWPRTFGQG | | | | | | | | | 2020/06/15/science.abc |
| | TKLEIK | | | | | | | | | 7424) |
| mAb-95 | DIVLTQSPLSLPVGLGQSASISCRSSQR | IGHV1-69 | IGHJ3 | IGKV2-30 | IGKJ2 | 3072 | ARDPSILNTG | 4262 | MQGTEW | Anna Wec et al., 2020 |
| | VVHTDGNTYLHWFHQRPGQSPRRLIY | (Human) | (Human) | (Human) | (Human) | | NHHWYDLDI | | PRT | (https://science.science |
| | KVSNRDSGVPDRFSGSGSGTDFTLRIS | | | | | | | | | mag.org/content/early/ |
| | RVEAEDVGVYYCMQGTEWPRTFGQ | | | | | | | | | 2020/06/15/science.abc |
| | GTKLEIK | | | | | | | | | 7424) |
| mAb-96 | ETTLTQSPVTLSVSPGERATLSCRASQS | IGHV3-66 | IGHJ5 | IGKV3-15 | IGKJ4 | 3073 | VRMDWME | 4263 | QQYNNWP | Anna Wec et al., 2020 |
| | VISNLAWYQQKPGQAPRLLIFGASTRA | (Human) | (Human) | (Human) | (Human) | | WMKYYFDS | | SLT | (https://science.science |
| | TGVPARFSGSGSGTEFTLTISSLQSEDF | | | | | | | | | mag.org/content/early/ |
| | AVYYCQQYNNWPSLTFGGGTKLEIK | | | | | | | | | 2020/06/15/science.abc |
| | | | | | | | | | | 7424) |
| mAb-97 | DIQVTQSPLSLPVGLGQSASISCRSSQR | IGHV1-69 | IGHJ3 | IGKV2-30 | IGKJ3 | 3074 | ARDPSILNTG | 4264 | MQATEWP | Anna Wec et al., 2020 |
| | VVHTDGNTYLNWFHQRPGQSPRRLIY | (Human) | (Human) | (Human) | (Human) | | NHHWYDLD | | RT | (https://science.science |
| | KVSNRDSGVPDRFSGSGSGTDFTLRIS | | | | | | M | | | mag.org/content/early/ |
| | RVEAEDVGVYYCMQATEWPRTFGQG | | | | | | | | | 2020/06/15/science.abc |
| | TKVDIK | | | | | | | | | 7424) |
| mAb-98 | EIVMTQSPATLSLSPGERATLSCRASQS | IGHV3-30 | IGHJ5 | IGKV3-11 | IGKJ2 | 3075 | VRDDVLQHS | 4265 | QQRSDGY | Anna Wec et al., 2020 |
| | VSTFLGWYQQRPGQPPRLLIYDASYR | (Human) | (Human) | (Human) | (Human) | | RPSGPGYFVS | | N | (https://science.science |
| | APDIPVRFSGSGSGTDFTLTINSLEPED | | | | | | | | | mag.org/content/early/ |
| | SAVYYCQQRSDGYNFGPGTKLEIK | | | | | | | | | 2020/06/15/science.abc |
| | | | | | | | | | | 7424) |
| mAb-99 | QPVLTQPPSVSVSPGQTARITCSGDAL | IGHV4-4 | IGHJ6 | IGLV3-10 | IGLJ3 | 3076 | ATMWGGLCT | 4266 | YSTDSTAN | Anna Wec et al., 2020 |
| | PRRYAYWYQQRSGQAPVLVIYEDNKR | (Human) | (Human) | (Human) | (Human) | | ASNCYGNPM | | YKV | (https://science.science |
| | PSGIPERFSAFSSGTRATLTISGAQVED | | | | | | DV | | | mag.org/content/early/ |
| | QADYYCYSTDSTANYKVFGGGTKLTVL | | | | | | | | | 2020/06/15/science.abc |
| | | | | | | | | | | 7424) |
| mBG17 | DIVMSQSPSSLAVSVGEKITMSCKSSQ | IGHV6-6 | IGHJ4 | IGKV8-30 | IGKJ1 | 3077 | TRSAMDY | 4267 | QQFYNYPR | James Terry et al., 2020 |
| | SLLYTSDQKNYLAWFQQKPGQSPKLLI | (Mouse) | (Mouse) | (Mouse) | (Mouse) | | | | T | (https://www.biorxiv.or |
| | FWASTRDSGVPDRFTGSGSGTDFTLTI | | | | | | | | | g/content/10.1101/202 |
| | SSVKAEDLAVYYCQQFYNYPRTFGGG | | | | | | | | | 0.09.03.280370v1.full) |
| | TKLEIK | | | | | | | | | |
| mBG21 | IVMTQTPKFLLVSAGDRVTITCKASQS | IGHV9-2- | IGHJ3 | IGKV6-32 | IGKJ1 | 3078 | ALRR | 4268 | QQDYSSP | James Terry et al., 2020 |
| | VSNDVAWFQQKPGQSPKKLLIYFASN | 1 | (Mouse) | (Mouse) | (Mouse) | | | | | (https://www.biorxiv.or |
| | RYTGVPDRFTGSGYGTDFTFTITTVQA | (Mouse) | | | | | | | | g/content/10.1101/202 |
| | EDLAVYFCQQDYSSPWTFGGGTKLEIK | | | | | | | | | 0.09.03.280370v1.full) |
| mBG22 | IVMTQTPKFLLVSAGDRVTITCKASQS | IGHV9-2- | IGHJ3 | IGKV6-32 | IGKJ1 | 3079 | ALRR | 4269 | QQDYSSP | James Terry et al., 2020 |
| | VSNDVAWFQQKPGQSPKLLIYFASNR | 1 | (Mouse) | (Mouse) | (Mouse) | | | | | (https://www.biorxiv.or |
| | YTGVPDRFTGSGYGTDFTFTITTVQAE | (Mouse) | | | | | | | | g/content/10.1101/202 |
| | DLAVYFCQQDYSSPWTFGGGTKLEIK | | | | | | | | | 0.09.03.280370v1.full) |
| mBG57 | DIVMTQAAPSEPVTPGESVSISCGSSK | IGHV9-2- | IGHJ3 | IGKV2-137 | IGKJ5 | 3080 | ALRR | 4270 | MQHLENP | James Terry et al., 2020 |
| | SLLHSNDNTYLYWFLQRPGQSPQLLIY | 1 | (Mouse) | (Mouse) | (Mouse) | | | | LGVR | (https://www.biorxiv.or |
| | RMSNLASGVPDRFTGSGSGTAFTLRIS | (Mouse) | | | | | | | | g/content/10.1101/202 |
| | RVEAEDVGVYYCMQHLENPLGVRWR | | | | | | | | | 0.09.03.280370v1.full) |
| | HQAGNQT | | | | | | | | | |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| mBG67 | Ab | SARS-CoV1, | 229E | | | N | Immunised Mouse | 975 | EVQLVESGGGLVQPGGSLKLSCAASGFT | 2042 |
| | | SARS-CoV2, | | | | | | | FSNYGMSWVRQTPDKRLELVATINRNG | |
| | | NL63 | | | | | | | GSTYYLDSVKVRFTISRDNAKSTLFLQLSS | |
| | | (weak) | | | | | | | LKSDDTAMYYCARIYDFDEDYFDVWGA | |
| | | | | | | | | | GTTVTVSS | |
| MD17 | Ab | SARS-CoV2 | | SARS-CoV2 | | S; RBD | Phage Display | | ND | |
| | | | | (weak) | | | Library (Antibody, | | | |
| | | | | | | | human, immune- | | | |
| | | | | | | | CoV2) | | | |
| MD29 | Ab | SARS-CoV2 | | SARS-CoV2 | | S; RBD | Phage Display | | ND | |
| | | | | (weak) | | | Library (Antibody, | | | |
| | | | | | | | human, immune- | | | |
| | | | | | | | CoV2) | | | |
| MD45 | Ab | SARS-CoV2 | | SARS-CoV2 | | S; RBD | Phage Display | | ND | |
| | | | | | | | Library (Antibody, | | | |
| | | | | | | | human, immune- | | | |
| | | | | | | | CoV2) | | | |
| MD47 | Ab | SARS-CoV2 | | SARS-CoV2 | | S; RBD | Phage Display | | ND | |
| | | | | (weak) | | | Library (Antibody, | | | |
| | | | | | | | human, immune- | | | |
| | | | | | | | CoV2 | | | |
| MD62 | Ab | SARS-CoV2 | | SARS-CoV2 | | S; RBD | Phage Display | | ND | |
| | | | | | | | Library (Antibody, | | | |
| | | | | | | | human, immune- | | | |
| | | | | | | | CoV2) | | | |
| MD63 | Ab | SARS-CoV2 | | | SARS-CoV2 | S; RBD | Phage Display | | ND | |
| | | | | | | | Library (Antibody, | | | |
| | | | | | | | human, immune- | | | |
| | | | | | | | CoV2) | | | |
| MD65 | Ab | SARS-CoV2 | | SARS-CoV2 | | S; RBD | Phage Display | | ND | |
| | | | | | | | Library (Antibody, | | | |
| | | | | | | | human, immune- | | | |
| | | | | | | | CoV2) | | | |
| MD67 | Ab | SARS-CoV2 | | SARS-CoV2 | | S; RBD | Phage Display | | ND | |
| | | | | | | | Library (Antibody, | | | |
| | | | | | | | human, immune- | | | |
| | | | | | | | CoV2) | | | |
| mNb6 | Nb | SARS-CoV2 | | SARS-CoV2 | | S; RBD | Yeast Display | | ND | |
| | | | | | | | Library (scFv, | | | |
| | | | | | | | human) | | | |
| mNb6-tri | Nb | SARS-CoV2 | | SARS-CoV2 | | S; RBD | Yeast Display | | ND | |
| | | | | | | | Library (scFv, | | | |
| | | | | | | | human) | | | |
| MnC1t3p1_ | Ab | SARS-CoV2 | | SARS-CoV2 | | S; RBD | B-cells; SARS-CoV2 | 976 | EVQLLESGGGLVQPGGSLRLSCAASGFT | 2043 |
| G9 | | | | | | | Human Patient | | FRNYAMTWVRQAPGKGLEWVSGISDS | |
| | | | | | | | | | GDRTYNADSVKGRFSISRDNSKNTLHLQ | |
| | | | | | | | | | MNSLRAEDTAVYYCALASGSYFGGANY | |
| | | | | | | | | | WGQGTLVTVSS | |
| MnC2t1p1_ | Ab | SARS-CoV2 | | SARS-CoV2 | | S; RBD | B-cells; SARS-CoV2 | 977 | EVQLVESGGGLVQPGGSLRLSCAASGFT | 2044 |
| A3 | | | | | | | Human Patient | | VSSNYMSWVRQAPGRGLEWVSVIYSG | |
| | | | | | | | | | GSTFYADSVKGRFTISRDNSKNTLYLQM | |
| | | | | | | | | | NSLRPEDTAVYYCATGARFGESPFDYW | |
| | | | | | | | | | GQGTLVTVSS | |
| MnC2t1p1_ | Ab | SARS-CoV2 | | SARS-CoV2 | | S; RBD | B-cells; SARS-CoV2 | 978 | EVQLVESGGGLVQPGGSLRLSCAASGFT | 2045 |
| C5 | | | | | | | Human Patient | | VSSNYMSWVRQAPGKGLEWVSVIYSG | |
| | | | | | | | | | GSTYYADSVKGRFTISRDNSKNTLYLQM | |
| | | | | | | | | | NSLRAEDTAVYYCATGARFGESPFDYW | |
| | | | | | | | | | GQGTLVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| mBG67 | QIVLTQSPAIMSASLGERVTMTCTASS | IGHV5-6- | IGHJ1 | IGKV4-74 | IGKJ1 | 3081 | ARIYDFDEDY | 4271 | LQYHRSP | James Terry et al., 2020 |
| | SVSSSYLHWYQQKPGSSPKLWIYSTSN | 3 | (Mouse) | (Mouse) | (Mouse) | | FDV | | | (https://www.biorxiv.or |
| | LASGVPARFSGSGSGTSYSLTISSMEAE | (Mouse) | | | | | | | | g/content/10.1101/202 |
| | DAATYYCLQYHRSPWTFGGGTKLEIK | | | | | | | | | 0.09.03.280370v1.full) |
| MD17 | ND | IGHV3-64 | ND | IGKV1-39 | ND | 3082 | VKDQDSSSW | 4272 | QQSYTTPL | Tal Noy-Porat et al., |
| | | (Human) | | (Human) | | | YDAFDI | | T | 2020 |
| | | | | | | | | | | (https://www.biorxiv.or |
| | | | | | | | | | | g/content/10.1101/202 |
| | | | | | | | | | | 0.05.20.106609v2) |
| MD29 | ND | IGHV3-64 | ND | IGKV1-39 | ND | 3083 | VKDQDSSSW | 4273 | HQTYTSPY | Tal Noy-Porat et al., |
| | | (Human) | | (Human) | | | YDAFDI | | T | 2020 |
| | | | | | | | | | | (https://www.biorxiv.or |
| | | | | | | | | | | g/content/10.1101/202 |
| | | | | | | | | | | 0.05.20.106609v2) |
| MD45 | ND | IGHV3-53 | ND | IGKV3-20 | ND | 3084 | ARDLSVRGG | 4274 | QQYGVSPE | Tal Noy-Porat et al., |
| | | (Human) | | (Human) | | | MDV | | II | 2020 |
| | | | | | | | | | | (https://www.biorxiv.or |
| | | | | | | | | | | g/content/10.1101/202 |
| | | | | | | | | | | 0.05.20.106609v2) |
| MD47 | ND | IGHV3-23 | ND | IGLV3-21 | ND | 3085 | AKDLVTAPSY | 4275 | QVWDSSS | Tal Noy-Porat et al., |
| | | (Human) | | (Human) | | | EAFDI | | HHHVV | 2020 |
| | | | | | | | | | | (https://www.biorxiv.or |
| | | | | | | | | | | g/content/10.1101/202 |
| | | | | | | | | | | 0.05.20.106609v2) |
| MD62 | ND | IGHV3-53 | ND | IGKV1-12 | ND | 3086 | ARDLQYYGM | 4276 | QQANSFPL | Tal Noy-Porat et al., |
| | | (Human) | | (Human) | | | DV | | T | 2020 |
| | | | | | | | | | | (https://www.biorxiv.or |
| | | | | | | | | | | g/content/10.1101/202 |
| | | | | | | | | | | 0.05.20.106609v2) |
| MD63 | ND | IGHV3-64 | ND | IGKV1-39 | ND | 3087 | VKDQDSNSW | 4277 | QQSYSTPY | Tal Noy-Porat et al., |
| | | (Human) | | (Human) | | | YDAFDI | | T | 2020 |
| | | | | | | | | | | (https://www.biorxiv.or |
| | | | | | | | | | | g/content/10.1101/202 |
| | | | | | | | | | | 0.05.20.106609v2) |
| MD65 | ND | IGHV3-66 | ND | IGKV3-20 | ND | 3088 | ARDLAVAGAF | 4278 | QQYGSSPL | Tal Noy-Porat et al., |
| | | (Human) | | (Human) | | | DI | | T | 2020 |
| | | | | | | | | | | (https://www.biorxiv.or |
| | | | | | | | | | | g/content/10.1101/202 |
| | | | | | | | | | | 0.05.20.106609v2) |
| MD67 | ND | IGHV3-53 | ND | IGKV3-20 | ND | 3089 | ARDLSVRGG | 4279 | QQFGSSPL | Tal Noy-Porat et al., |
| | | (Human) | | (Human) | | | MDV | | T | 2020 |
| | | | | | | | | | | (https://www.biorxiv.or |
| | | | | | | | | | | g/content/10.1101/202 |
| | | | | | | | | | | 0.05.20.106609v2) |
| mNb6 | ND | ND | ND | ND | ND | | ND | | ND | Michael Schoof et al., |
| | | | | | | | | | | 2020 |
| | | | | | | | | | | (https://www.biorxiv.or |
| | | | | | | | | | | g/content/10.1101/202 |
| | | | | | | | | | | 0.08.08.238469v1.full.p |
| | | | | | | | | | | df) |
| mNb6-tri | ND | ND | ND | ND | ND | | ND | | ND | Michael Schoof et al., |
| | | | | | | | | | | 2020 |
| | | | | | | | | | | (https://www.biorxiv.or |
| | | | | | | | | | | g/content/10.1101/202 |
| | | | | | | | | | | 0.08.08.238469v1.full.p |
| | | | | | | | | | | df) |
| MnC1t3p1_ | QAVVTQEPSLTVSPGGTVTLTCGSSTG | IGHV3-23 | IGHJ4 | IGLV7-46 | IGLJ3 | 3090 | ALASGSYFGG | 4280 | LLSYTGAR | Christoph Kreer et al., |
| G9 | PVTSDHYPYWFQQKPGQAPTTLIYDT | (Human) | (Human) | (Human) | (Human) | | ANY | | V | 2020 |
| | NNKHSWTPARFSGSLLGGKAALTLSG | | | | | | | | | (https://doi.org/10.101 |
| | AQPEDEAEYYCLLSYTGARVFGGGTKL | | | | | | | | | 6/j.cell.2020.06.044) |
| | TVL | | | | | | | | | |
| MnC2t1p1_ | DIQMTQSPSSVSASVGDRVTITCRASQ | IGHV3-66 | IGHJ4 | IGKV1D-12 | IGKJ1 | 3091 | ATGARFGESP | 4281 | QQANSFP | Christoph Kreer et al., |
| A3 | GISSWLAWYQQKPGKAPKLLIYAASSL | (Human) | (Human) | (Human) | (Human) | | FDY | | GT | 2020 |
| | QSGVPSRFSGSGSGTDFTLTISSLQPED | | | | | | | | | (https://doi.org/10.101 |
| | FATYYCQQANSFPGTFGQGTKVEIK | | | | | | | | | 6/j.cell.2020.06.044) |
| MnC2t1p1_ | DIQMTQSPSSVSASVGDRVTITCRASQ | IGHV3-66 | IGHJ4 | IGKV1D-12 | IGKJ1 | 3092 | ATGARFGESP | 4282 | QQANSFP | Christoph Kreer et al., |
| C5 | GISSWLAWYQQKPGKAPKLLIYAASSL | (Human) | (Human) | (Human) | (Human) | | FDY | | GT | 2020 |
| | QSGVPSRFSGSGSGTDFTLTISSLOPED | | | | | | | | | (https://doi.org/10.101 |
| | FATYYCQQANSFPGTFGQGTKVEIK | | | | | | | | | 6/j.cell.2020.06.044) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| MnC2t2p1_ | Ab | SARS-CoV2 | | SARS-CoV2 | | S; RBD | B-cells; SARS-CoV2 | 979 | QVQLVQSGAEVKKPGSSVKVSCKASGG | 2046 |
| C11 | | | | | | | Human Patient | | TFSRYTIIWVRQAPGQGLEWMGRIIPIL | |
| | | | | | | | | | DIANYAQKFQGRVTITADKSTSTAYMEL | |
| | | | | | | | | | SSLRSEDTAVYYCAREGGLDYFGSRNSG | |
| | | | | | | | | | WTYTWFDPWGQGTLVTVSS | |
| MnC4t1p1_ | Ab | SARS-CoV2 | | SARS-CoV2 | | S; RBD | B-cells; SARS-CoV2 | 980 | QLQVQESGPGLVKPSETLSLTCTVSGASI | 2047 |
| A10 | | | | | | | Human Patient | | SSNHYFWGWIRQPPGKGLAWIGSMHY | |
| | | | | | | | | | SGSTYYNPSLKSRVTISVDTSKNQLSLKLS | |
| | | | | | | | | | SVTAADTAVYYCARGVNYYDRNGYYRN | |
| | | | | | | | | | DGFDIRGQGTMVTVSS | |
| MnC4t1p1_ | Ab | SARS-CoV2 | | SARS-CoV2 | | S; RBD | B-cells; SARS-CoV2 | 981 | EVQLVESGGGLVQPGGSLRLSCAASGFT | 2048 |
| A11 | | | | | | | Human Patient | | FSSYSMNWVRQAPGKGLEWVSYISSSS | |
| | | | | | | | | | NTRYYTDSVMGRFTISRDNAKNSLFLQ | |
| | | | | | | | | | MNSLRAEDTAVYYCASSKGFCSGGSCSD | |
| | | | | | | | | | YWGQGTLVTVSS | |
| MnC4t2p1_ | Ab | SARS-CoV2 | | SARS-CoV2 | | S; RBD | B-cells; SARS-CoV2 | 982 | EVQLVESGGHLVQPGRSLRLSCAASGFT | 2049 |
| B3 | | | | | | | Human Patient | | FDDYAMHWVRQVPGKGLEWVSGISW | |
| | | | | | | | | | NGGILDYADSVKGRFTISRDNAKNSLYL | |
| | | | | | | | | | HMRSLRTDDTALYYCAKDLRRQDYYAD | |
| | | | | | | | | | WYFDLWGRGTLVTVSS | |
| MnC4t2p1_ | Ab | SARS-CoV2 | | SARS-CoV2 | | S; RBD | B-cells; SARS-CoV2 | 983 | QLQVQESGPGLVKPSETLSLTCTVSGASI | 2050 |
| D10 | | | | | | | Human Patient | | SSNHYFWGWIRQPPGKGLAWIGSMHY | |
| | | | | | | | | | SGSTYYNPSLKSRVTISVDTSKNQLSLKLS | |
| | | | | | | | | | SVTAADTAVYYCARGVNYYDRNGYYRN | |
| | | | | | | | | | DGFDIRGQGTMVTVSS | |
| MnC4t2p1_ | Ab | SARS-CoV2 | | SARS-CoV2 | | S; RBD | B-cells; SARS-CoV2 | 984 | EVQLVESGGHLVQPGRSLRLSCAASGFT | 2051 |
| E6 | | | | | | | Human Patient | | FDDYAMHWVRQVPGKGLEWVSGISW | |
| | | | | | | | | | NGGILGYADSVKGRFTISRDNAKNSLYL | |
| | | | | | | | | | QMRSLRTDDTALYYCAKDLRRQDYYAD | |
| | | | | | | | | | WYFDLWGRGTLVTVSS | |
| MnC4t2p1_ | Ab | SARS-CoV2 | | SARS-CoV2 | | S; RBD | B-cells; SARS-CoV2 | 985 | QLQVQESGPGLVKPSETLSLTCTVSGASI | 2052 |
| F5 | | | | | | | Human Patient | | SSNHYFWGWIRQPPGKGLAWIGSMHY | |
| | | | | | | | | | SGSTYYNPSLKSRVTISVDTSKNQLSLKLS | |
| | | | | | | | | | SVTAADTAVYYCARGVNYYDRNGYYRN | |
| | | | | | | | | | DGFDIRGQGTMVTVSS | |
| MnC4t2p2_ | Ab | SARS-CoV2 | | SARS-CoV2 | | S; RBD | B-cells; SARS-CoV2 | 986 | QVQLVQSGSELKKPGASVKISCKASGYIF | 2053 |
| A4 | | | | | | | Human Patient | | INYAMNWVRQAPGQGLEWMGWINT | |
| | | | | | | | | | NTGNPTYAQDFTGRFVFSLDTSLSTAYL | |
| | | | | | | | | | QISSLEAEDTAVYYCAKIGSRNSLGVWG | |
| | | | | | | | | | QGTLVTVSA | |
| MnC5t2p1_ | Ab | SARS-CoV2 | | SARS-CoV2 | | S; RBD | B-cells; SARS-CoV2 | 987 | QMQLVQSGPEVKKPGTSVKVSCKASGF | 2054 |
| G1 | | | | | | | Human Patient | | TFTSSAVQWVRQARGQRLEWIGWIVV | |
| | | | | | | | | | GSGNTDYAQKFQERVTITRDVSTSTAYM | |
| | | | | | | | | | ELSSLRSEDTAVYYCAAPRCSGGSCYDGF | |
| | | | | | | | | | DIWGQGTMVTVSS | |
| MR10 | Nb | SARS-CoV2 | | | | S; RBD | Phage Display | 988 | QVQLVESGGGLVQAGGSLRLSCAASGF | |
| | | | | | | | Library (Nanobody, | | PVANTWMEWYRQAPGKEREWVAAITS | |
| | | | | | | | non-immune) | | YGYRTYYADSVKGRFTISRDNAKNTVYL | |
| | | | | | | | | | QMNSLKPEDTAVYYCNVKDEGATTKVY | |
| | | | | | | | | | DYWGQGTQVTVSS | |
| MR14 | Nb | SARS-CoV2 | | | | S; RBD | Phage Display | 989 | QVQLVESGGGLVQAGGSLRLSCAASGF | |
| | | | | | | | Library (Nanobody, | | PVMYTHMHWYRQAPGKEREWVAAIV | |
| | | | | | | | non-immune) | | SLGEYTTYADSVKGRFTISRDNAKNTVYL | |
| | | | | | | | | | QMNSLKPEDTAVYYCNVKDWGAANKY | |
| | | | | | | | | | YDYWGQGTQVTVSS | |
| MR17 | Nb | SARS-CoV2 | | | | S; RBD | Phage Display | 990 | QVQLVESGGGLVQAGGSLRLSCAASGF | |
| | | | | | | | Library (Nanobody, | | PVEVWRMEWYRQAPGKEREGVAAIES | |
| | | | | | | | non-immune) | | YGHGTRYADSVKGRFTISRDNAKNTVYL | |
| | | | | | | | | | QMNSLKPEDTAVYYCNVKDDGQLAYHY | |
| | | | | | | | | | DYWGQGTQVTVSS | |
| MR17_ | Nb | SARS-CoV2 | | | | S; RBD | Derived from MR17 | 991 | QVQLVESGGGLVQAGGSLRLSCAASGF | |
| K56W | | | | | | | | | PVEVWRMEWYRQAPGKEREGVAAIES | |
| | | | | | | | | | YGWGTRYADSVKGRFTISRDNAKNTVYL | |
| | | | | | | | | | QMNSLKPEDTAVYYCNVKDDGQLAYHY | |
| | | | | | | | | | DYWGQGTQVTVSS | |
| MR17_ | Nb | SARS-CoV2 | | | | S; RBD | Derived from MR17 | 992 | QVQLVESGGGLVQAGGSLRLSCAASGF | |
| K99W | | | | | | | | | PVEVWRMEWYRQAPGKEREGVAAIES | |
| | | | | | | | | | YGHGTRYADSVKGRFTISRDNAKNTVYL | |
| | | | | | | | | | QMNSLKPEDTAVYYCNVWDDGQLAYH | |
| | | | | | | | | | YDYWGQGTQVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| MnC2t2p1_ | DIQMTQSPSSLSASVGDRVTITCRASQ | IGHV1-69 | IGHJ5 | IGKV1-39 | IGKJ2 | 3093 | AREGGLDYFG | 4283 | QQSYSTLY | Christoph Kreer et al., |
| C11 | NISSYLNWYQQKPGKAPNLLIYAASSL | (Human) | (Human) | (Human) | (Human) | | SRNSGWTYT | | S | 2020 |
| | QSGVPPRFSGSGSGTDFTLTISSLQPED | | | | | | WFDP | | | (https://doi.org/10.101 |
| | FATYYCQQSYSTLYSFGQGTKLEIK | | | | | | | | | 6/j.cell.2020.06.044) |
| MnC4t1p1_ | DIQMTQSPSSLSASVGDRVTITCRASQ | IGHV4-39 | IGHJ3 | IGKV1-17 | IGKJ3 | 3094 | ARGVNYYDR | 4284 | LQHNTYPF | Christoph Kreer et al., |
| A10 | GIRNDLGWYEQKPGKAPKRLIYAASSL | (Human) | (Human) | (Human) | (Human) | | NGYYRNDGF | | T | 2020 |
| | ESGVPSRFSGSGSGTEFTLTISSLQPED | | | | | | DI | | | (https://doi.org/10.101 |
| | FATYYCLQHNTYPFTFGPGTRVDIK | | | | | | | | | 6/j.cell.2020.06.044) |
| MnC4t1p1_ | EIVLTQSPGTLSLSPGERATLSCRASQS | IGHV3-48 | IGHJ4 | IGKV3-20 | IGKJ1 | 3095 | ASSKGFCSGG | 4285 | HQYGSSP | Christoph Kreer et al., |
| A11 | VSSSYLAWYQQKPGQAPRLLIYGVSSR | (Human) | (Human) | (Human) | (Human) | | SCSDY | | WT | 2020 |
| | ATGIPDRFSGSGSGTDFTLTISRLEPED | | | | | | | | | (https://doi.org/10.101 |
| | FVVYYCHQYGSSPWTFGQGTKVEIK | | | | | | | | | 6/j.cell.2020.06.044) |
| MnC4t2p1_ | DIQMIQSPSSVSASVGDRVTITCRASQ | IGHV3-9 | IGHJ2 | IGKV1-12 | IGKJ3 | 3096 | AKDLRRQDYY | 4286 | QQGNSFPF | Christoph Kreer et al., |
| B3 | GISSWLAWYQQKPGKAPKLLIYAASTL | (Human) | (Human) | (Human) | (Human) | | ADWYFDL | | T | 2020 |
| | LSAVPSRFSGSGSGTDFTLTISSLQPED | | | | | | | | | (https://doi.org/10.101 |
| | FATYYCQQGNSFPFTFGPGTIVDV | | | | | | | | | 6/j.cell.2020.06.044) |
| MnC4t2p1_ | DIQMTQSPSSLSASVGDRVTITCRASQ | IGHV4-39 | IGHJ3 | IGKV1-17 | IGKJ3 | 3097 | ARGVNYYDR | 4287 | LQHNTYPF | Christoph Kreer et al., |
| D10 | GIRNDLGWYEQKPGKAPKRLIYAASSL | (Human) | (Human) | (Human) | (Human) | | NGYYRNDGF | | T | 2020 |
| | ESGVPSRFSGSGSGTEFTLTISSLQPED | | | | | | DI | | | (https://doi.org/10.101 |
| | FATYYCLQHNTYPFTFGPGTRVDIK | | | | | | | | | 6/j.cell.2020.06.044) |
| MnC4t2p1_ | DIQMTQSPSSVSASVGDRVTITCRASQ | IGHV3-9 | IGHJ2 | IGKV1-12 | IGKJ3 | 3098 | AKDLRRQDYY | 4288 | QQGNSFPF | Christoph Kreer et al., |
| E6 | GISSWLAWYQQKPGKAPKLLIYAASTL | (Human) | (Human) | (Human) | (Human) | | ADWYFDL | | T | 2020 |
| | LSAVPSRFSGSGSGTDFTLTISSLQPED | | | | | | | | | (https://doi.org/10.101 |
| | FATYYCQQGNSFPFTFGPGTKVDV | | | | | | | | | 6/j.cell.2020.06.044) |
| MnC4t2p1_ | DIQMTQSPSSLSASVGDRVTITCRASQ | IGHV4-39 | IGHJ3 | IGKV1-17 | IGKJ3 | 3099 | ARGVNYYDR | 4289 | LQHNTYPF | Christoph Kreer et al., |
| F5 | GIRNDLGWYEQKPGKAPKRLIYAASSL | (Human) | (Human) | (Human) | (Human) | | NGYYRNDGF | | T | 2020 |
| | ESGVPSRFSGSGSGTEFTLTISSLQPED | | | | | | DI | | | (https://doi.org/10.101 |
| | FATYYCLQHNTYPFTFGPGTRVDIK | | | | | | | | | 6/j.cell.2020.06.044) |
| MnC4t2p2_ | EIVLTQSPGTLSLSPGERATLSCRASHS | IGHV7-4- | IGHJ4 | IGKV3-20 | IGKJ5 | 3100 | AKIGSRNSLG | 4290 | QHFGTSSV | Christoph Kreer et al., |
| A4 | VDRSYLAWYQQKPGLAPRLLIYGASSR | 1 | (Human) | (Human) | (Human) | | V | | T | 2020 |
| | ATGIPDRFSGSGSGTDFTLTISRLEPED | (Human) | | | | | | | | (https://doi.org/10.101 |
| | FALYYCQHFGTSSVTFGRGTRLEIK | | | | | | | | | 6/j.cell.2020.06.044) |
| MnC5t2p1_ | EIVLTQSPGTLSLSPGERATLSCRASQS | IGHV1-58 | IGHJ3 | IGKV3-20 | IGKJ1 | 3101 | AAPRCSGGSC | 4291 | QQYGSSP | Christoph Kreer et al., |
| G1 | VSSSYLAWYQHKPGQAPRLLICGASSR | (Human) | (Human) | (Human) | (Human) | | YDGFDI | | WT | 2020 |
| | ATGIPDRFSGSGSGTGFTLTISRLEPED | | | | | | | | | (https://doi.org/10.101 |
| | FAVYYCQQYGSSPWTFGQGTKVEIK | | | | | | | | | 6/j.cell.2020.06.044) |
| MR10 | N/A | IGHV3S5 | IGHJ4 | N/A | N/A | 3102 | NVKDEGATTK | | N/A | Tingting Li et al., 2020 |
| | | 3 | (Alpaca) | | | | VYDY | | | (https://www.biorxiv.or |
| | | (Alpaca) | | | | | | | | g/content/10.1101/202 |
| | | | | | | | | | | 0.06.09.143438v1) |
| MR14 | N/A | IGHV3S5 | IGHJ4 | N/A | N/A | 3103 | NVKDWGAA | | N/A | Tingting Li et al., 2020 |
| | | 3 | (Alpaca) | | | | NKYYDY | | | (https://www.biorxiv.or |
| | | (Alpaca) | | | | | | | | g/content/10.1101/202 |
| | | | | | | | | | | 0.06.09.143438v1) |
| MR17 | N/A | IGHV3S5 | IGHJ4 | N/A | N/A | 3104 | NVKDDGQLA | | N/A | Tingting Li et al., 2020 |
| | | 3 | (Alpaca) | | | | YHYDY | | | (https://www.biorxiv.or |
| | | (Alpaca) | | | | | | | | g/content/10.1101/202 |
| | | | | | | | | | | 0.06.09.143438v1) |
| MR17_ | N/A | IGHV3S5 | IGHJ4 | N/A | N/A | 3105 | NVKDDGQLA | | N/A | Tingting Li et al., 2020 |
| K56W | | 3 | (Alpaca) | | | | YHYDY | | | (https://www.biorxiv.or |
| | | (Alpaca) | | | | | | | | g/content/10.1101/202 |
| | | | | | | | | | | 0.06.09.143438v1) |
| MR17_ | N/A | IGHV3S5 | IGHJ4 | N/A | N/A | 3106 | NVWDDGQL | | N/A | Tingting Li et al., 2020 |
| K99W | | 3 | (Alpaca) | | | | AYHYDY | | | (https://www.biorxiv.or |
| | | (Alpaca) | | | | | | | | g/content/10.1101/202 |
| | | | | | | | | | | 0.06.09.143438v1) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| MR17_ | Nb | SARS-CoV2 | | | | S; RBD | Derived from MR17 | 993 | QVQLVESGGGLVQAGGSLRLSCAASGF | |
| K99Y | | | | | | | | | PVEVWRMEWYRQAPGKEREGVAAIES | |
| | | | | | | | | | YGHGTRYADSVKGRFTISRDNAKNTVYL | |
| | | | | | | | | | QMNSLKPEDTAVYYCNVYDDGQLAYHY | |
| | | | | | | | | | DYWGQGTQVTVSS | |
| MR2 | Nb | SARS-CoV2 | | | | S; RBD | Phage Display | 994 | QVQLVESGGGLVQAGGSLRLSCAASGF | |
| | | | | | | | Library (Nanobody, | | PVYFSYMAWYRQAPGKEREWVAAINSE | |
| | | | | | | | non-immune) | | GDSTTYADSVKGRFTISRDNAKNTVYLQ | |
| | | | | | | | | | MNSLKPEDTAVYYCNVKDYGWYNSQY | |
| | | | | | | | | | DYWGQGTQVTVSS | |
| MR3 | Nb | SARS-CoV2 | | | | S; RBD | Phage Display | 995 | QVQLVESGGGLVQAGGSLRLSCAASGF | |
| | | | | | | | Library (Nanobody, | | PVNAHFMYWYRQAPGKEREWVAAIYS | |
| | | | | | | | non-immune) | | YGRTLYADSVKGRFTISRDNAKNTVYLQ | |
| | | | | | | | | | MNSLKPEDTAVYYCNVKDYGAASWEYD | |
| | | | | | | | | | YWGQGTQVTVSS | |
| MR4 | Nb | SARS-CoV2 | | | | S; RBD | Phage Display | 996 | QVQLVESGGGLVQAGGSLRLSCAASGF | |
| | | | | | | | Library (Nanobody, | | PMYAWEMAWYRQAPGKEREWVAAIR | |
| | | | | | | | non-immune) | | SMGVHTHYSDSVKGRFTISRDNAKNTV | |
| | | | | | | | | | YLQMNSLKPEDTAVYYCNVKDFGGHQA | |
| | | | | | | | | | YYDYWGQGTQVTVSS | |
| MR6 | Nb | SARS-CoV2 | | | | S; RBD | Phage Display | 997 | QVQLVESGGGLVQAGGSLRLSCAASGF | |
| | | | | | | | Library (Nanobody, | | PVEDTWMEWYRQAPGKEREWVAAITS | |
| | | | | | | | non-immune) | | WGFKTYYADSVKGRFTISRDNAKNTVYL | |
| | | | | | | | | | QMNSLKPEDTAVYYCNVKDEGDTSASY | |
| | | | | | | | | | DYWGQGTQVTVSS | |
| MR7 | Nb | SARS-CoV2 | | | | S; RBD | Phage Display | 998 | QVQLVESGGGLVQAGGSLRLSCAASGF | |
| | | | | | | | Library (Nanobody, | | PVNSWMEWYRQAPGKEREWVAAITSY | |
| | | | | | | | non-immune) | | GYKTYYADSVKGRFTISRDNAKNTVYLQ | |
| | | | | | | | | | MNSLKPEDTAVYYCNVKDEGYFSDEYDY | |
| | | | | | | | | | WGQGTQVTVSS | |
| MR8 | Nb | SARS-CoV2 | | | | S; RBD | Phage Display | 999 | QVQLVESGGGLVQAGGSLRLSCAASGF | |
| | | | | | | | Library (Nanobody, | | PVEWAHMHWYRQAPGKEREWVAAIV | |
| | | | | | | | non-immune) | | SAGHYTVYADSVKGRFTISRDNAKNTVY | |
| | | | | | | | | | LQMNSLKPEDTAVYYCNVKDWGSSNQ | |
| | | | | | | | | | YYDYWGQGTQVTVSS | |
| Nb11 | Nb | SARS-CoV2 | | SARS-CoV2 | | S; RBD | Yeast Display | | ND | |
| | | | | | | | Library (scFv, | | | |
| | | | | | | | human) | | | |
| Nb11-59 | Nb | SARS-CoV1, | | SARS-CoV1, | | S; RBD | Phage Display | | ND | |
| | | SARS-CoV2 | | SARS-CoV2 | | | (Immunised Camel) | | | |
| Nb3 | Nb | SARS-CoV2 | | SARS-CoV2 | | S; RBD | Yeast Display | | ND | |
| | | | | | | | Library (scFv, | | | |
| | | | | | | | human) | | | |
| Nb3-bi | Nb | SARS-CoV2 | | SARS-CoV2 | | S; RBD | Yeast Display | | ND | |
| | | | | | | | Library (scFv, | | | |
| | | | | | | | human) | | | |
| Nb3-tri | Nb | SARS-CoV2 | | SARS-CoV2 | | S; RBD | Yeast Display | | ND | |
| | | | | | | | Library (scFv, | | | |
| | | | | | | | human) | | | |
| Nb4-43 | Nb | SARS-CoV2 | SARS- | SARS-CoV2 | SARS-CoV1 | S; RBD | Phage Display | | ND | |
| | | | CoV1 | | | | (Immunised Camel) | | | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| MR17_ | N/A | IGHV3S5 | IGHJ4 | N/A | N/A | 3107 | NVYDDGQLA | | N/A | Tingting Li et al., 2020 |
| K99Y | | 3 | (Alpaca) | | | | YHYDY | | | (https://www.biorxiv.or |
| | | (Alpaca) | | | | | | | | g/content/10.1101/202 |
| | | | | | | | | | | 0.06.09.143438v1) |
| MR2 | N/A | IGHV3S5 | IGHJ4 | N/A | N/A | 3108 | NVKDYGWYN | | N/A | Tingting Li et al., 2020 |
| | | 3 | (Alpaca) | | | | SQYDY | | | (https://www.biorxiv.or |
| | | (Alpaca) | | | | | | | | g/content/10.1101/202 |
| | | | | | | | | | | 0.06.09.143438v1) |
| MR3 | N/A | IGHV3S5 | IGHJ4 | N/A | N/A | 3109 | NVKDYGAAS | | N/A | Tingting Li et al., 2020 |
| | | 3 | (Alpaca) | | | | WEYDY | | | (https://www.biorxiv.or |
| | | (Alpaca) | | | | | | | | g/content/10.1101/202 |
| | | | | | | | | | | 0.06.09.143438v1) |
| MR4 | N/A | IGHV3S5 | IGHJ4 | N/A | N/A | 3110 | NVKDFGGHQ | | N/A | Tingting Li et al., 2020 |
| | | 3 | (Alpaca) | | | | AYYDY | | | (https://www.biorxiv.or |
| | | (Alpaca) | | | | | | | | g/content/10.1101/202 |
| | | | | | | | | | | 0.06.09.143438v1) |
| MR6 | N/A | IGHV3S5 | IGHJ4 | N/A | N/A | 3111 | NVKDEGDTS | | N/A | Tingting Li et al., 2020 |
| | | 3 | (Alpaca) | | | | ASYDY | | | (https://www.biorxiv.or |
| | | (Alpaca) | | | | | | | | g/content/10.1101/202 |
| | | | | | | | | | | 0.06.09.143438v1) |
| MR7 | N/A | IGHV3S5 | IGHJ4 | N/A | N/A | 3112 | NVKDEGYFSD | | N/A | Tingting Li et al., 2020 |
| | | 3 | (Alpaca) | | | | EYDY | | | (https://www.biorxiv.or |
| | | (Alpaca) | | | | | | | | g/content/10.1101/202 |
| | | | | | | | | | | 0.06.09.143438v1) |
| MR8 | N/A | IGHV3S5 | IGHJ4 | N/A | N/A | 3113 | NVKDWGSSN | | N/A | Tingting Li et al., 2020 |
| | | 3 | (Alpaca) | | | | QYYDY | | | (https://www.biorxiv.or |
| | | (Alpaca) | | | | | | | | g/content/10.1101/202 |
| | | | | | | | | | | 0.06.09.143438v1) |
| Nb11 | ND | ND | ND | ND | ND | | ND | | ND | Michael Schoof et al., |
| | | | | | | | | | | 2020 |
| | | | | | | | | | | (https://www.biorxiv.or |
| | | | | | | | | | | g/content/10.1101/202 |
| | | | | | | | | | | 0.08.08.238469v1.full.p |
| | | | | | | | | | | df) |
| Nb11-59 | ND | ND | ND | ND | ND | | ND | | ND | Junwei Gai et al., 2020 |
| | | | | | | | | | | (https://www.biorxiv.or |
| | | | | | | | | | | g/content/10.1101/202 |
| | | | | | | | | | | 0.08.09.242867v1.full.p |
| | | | | | | | | | | df) |
| Nb3 | ND | ND | ND | ND | ND | | ND | | ND | Michael Schoof et al., |
| | | | | | | | | | | 2020 |
| | | | | | | | | | | (https://www.biorxiv.or |
| | | | | | | | | | | g/content/10.1101/202 |
| | | | | | | | | | | 0.08.08.238469v1.full.p |
| | | | | | | | | | | df) |
| Nb3-bi | ND | ND | ND | ND | ND | | ND | | ND | Michael Schoof et al., |
| | | | | | | | | | | 2020 |
| | | | | | | | | | | (https://www.biorxiv.or |
| | | | | | | | | | | g/content/10.1101/202 |
| | | | | | | | | | | 0.08.08.238469v1.full.p |
| | | | | | | | | | | df) |
| Nb3-tri | ND | ND | ND | ND | ND | | ND | | ND | Michael Schoof et al., |
| | | | | | | | | | | 2020 |
| | | | | | | | | | | (https://www.biorxiv.or |
| | | | | | | | | | | g/content/10.1101/202 |
| | | | | | | | | | | 0.08.08.238469v1.full.p |
| | | | | | | | | | | df) |
| Nb4-43 | ND | ND | ND | ND | ND | | ND | | ND | Junwei Gai et al., 2020 |
| | | | | | | | | | | (https://www.biorxiv.or |
| | | | | | | | | | | g/content/10.1101/202 |
| | | | | | | | | | | 0.08.09.242867v1.full.p |
| | | | | | | | | | | df) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| Nb6 | Nb | SARS-CoV2 | | SARS-CoV2 | | S; RBD | Yeast Display | | ND | |
| | | | | | | | Library (scFv, | | | |
| | | | | | | | human) | | | |
| Nb6-tri | Nb | SARS-CoV2 | | SARS-CoV2 | | S; RBD | Yeast Display | | ND | |
| | | | | | | | Library (scFv, | | | |
| | | | | | | | human) | | | |
| NIH- | Nb | SARS-CoV2 | | | | S; RBD | Immunised Llama | 1000 | DVQLQESGGDLVQPGGSLRLSCAASGF | |
| CoVnb- | | | | | | | | | TLDYYAIGWFRQAPGKEREGVSCISSSD | |
| 101 | | | | | | | | | GSTYYADSVKGRFTSSRDNAKNTVYLQ | |
| | | | | | | | | | MNSLKPEDTAVYYCAAVPSTYYNGSYYY | |
| | | | | | | | | | TCHPGGMDYWGKGTQVTVSS | |
| NIH- | Nb | SARS-CoV2 | | | | S; RBD | Immunised Llama | 1001 | DVQLQESGGGLVQPGGSLRLSCAVSGF | |
| CoVnb- | | | | | | | | | TLDYYAIGWFRQAPGKEREGVSCISSSD | |
| 102 | | | | | | | | | GSTYYADSVKGRFTSSRDNAKNTVYLQ | |
| | | | | | | | | | MNSLKPEDTAVYYCAAVPSTYYSGTYYY | |
| | | | | | | | | | NCHPGGMDYWGKGTQVTVSS | |
| NIH- | Nb | SARS-CoV2 | | | | S; RBD | Immunised Llama | 1002 | DVQLQESGGGLVQPGGSLRLSCAASGL | |
| CoVnb- | | | | | | | | | TLDYYTIGWFRQAPGKEREGVSCISSSD | |
| 103 | | | | | | | | | DSTYYADSVKGRFTISRDNAKNTVYLQM | |
| | | | | | | | | | NSLKPEDTAVYYCATAPGTYYKGSYYPM | |
| | | | | | | | | | CHYYGMDYWGKGTQVTVSS | |
| NIH- | Nb | SARS-CoV2 | | | | S; RBD | Immunised Llama | 1003 | DVQLQESGGGLVQPGGSLRLSCAVSGF | |
| CoVnb- | | | | | | | | | TLDYYAIGWFRQAPGKEREGVACISSSD | |
| 104 | | | | | | | | | GTTYYADSVKGRFTISRDNAKNTVYLQM | |
| | | | | | | | | | NSLKPEDTAVYYCATRPLTYYSGSYYTTCS | |
| | | | | | | | | | DYGMDYWGKGTLVTVSS | |
| NIH | Nb | SARS-CoV2 | | | | S; RBD | Immunised Llama | 1004 | DVQLQESGGGLVQPGGSLRLSCAASGF | |
| CoVnb- | | | | | | | | | TLDYYAIGWFRQAPGKEREGVSCISNSD | |
| 105 | | | | | | | | | GSTYYADSVKGRFTTSRDNAKNTVYLQ | |
| | | | | | | | | | MNSLKPEDTAVYYCAAVPSTYYSGSYYYT | |
| | | | | | | | | | CHPGGMDYWGKGTQVTVSS | |
| NIH- | Nb | SARS-CoV2 | | | | S; RBD | Immunised Llama | 1005 | DVQLQESGGGLVQSGGSLRLSCAASGF | |
| CoVnb- | | | | | | | | | TLDYYAIGWFRQAPGKEREGVSCISNSD | |
| 106 | | | | | | | | | GSTYYADSVKGRFTTSRDNAKNTVYLQ | |
| | | | | | | | | | MNSLKPEDTAVYYCAAVPSTYYSGSYYYT | |
| | | | | | | | | | CHPGGMDYWGKGTQVTVSS | |
| NIH- | Nb | SARS-CoV2 | | | | S; RBD | Immunised Llama | 1006 | DVQLQESGGGLVQPGGSLRLSCAASGF | |
| CoVnb- | | | | | | | | | TLDYYAIGWFRQAPGKEREGVSCISNSD | |
| 107 | | | | | | | | | GSTYYADSVKGRFTTSRDNAKNTVYLQ | |
| | | | | | | | | | MNSLKPEDTAVYYCAAVPSTYYSGSYYYT | |
| | | | | | | | | | CHPGGMDYWGKGTLVTVSS | |
| NIH- | Nb | SARS-CoV2 | | | | S; RBD | Immunised Llama | 1007 | DVQLQESGGGLVQPGGSLRLSCAASGF | |
| CoVnb- | | | | | | | | | TLDYYAIGWFRQAPGKEREGVSCISNSG | |
| 108 | | | | | | | | | GSTYYADSVKGRFTTSRDNAKNTVYLQ | |
| | | | | | | | | | MNSLKPEDTAVYYCAAVPSTYYSGSYYYT | |
| | | | | | | | | | CHPGGMDYWGKGTQVTVSS | |
| NIH- | Nb | SARS-CoV2 | | | | S; RBD | Immunised Llama | 1008 | DVQLQESGGGLVQSGGSLRLSCAASGF | |
| CoVnb- | | | | | | | | | TLDYYAIGWFRQAPGKEREGVSCITNSD | |
| 109 | | | | | | | | | GSTYYADSVKGRFTTSRDNAKNTVYLQ | |
| | | | | | | | | | MNSLKPEDTAVYYCASFPSTYYSGSYYYT | |
| | | | | | | | | | CHPGGMDYWGKGTQVTVSS | |
| NIH- | Nb | SARS-CoV2 | | | | S; RBD | Immunised Llama | 1009 | DVQLQESGGGLVQPGGSLRLSCAASGF | |
| CoVnb- | | | | | | | | | TLDYYAIGWFRQAPGKEREGVSCISSSD | |
| 110 | | | | | | | | | GSTYYADSVKGRFTISRDNAKNTVYLQM | |
| | | | | | | | | | NSLKPDDTAVYYCAAALSEGGYTIDGSS | |
| | | | | | | | | | WCYHSVYGMDYWGKGTQVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| Nb6 | ND | ND | ND | ND | ND | | ND | | ND | Michael Schoof et al., |
| | | | | | | | | | | 2020 |
| | | | | | | | | | | (https://www.biorxiv.or |
| | | | | | | | | | | g/content/10.1101/202 |
| | | | | | | | | | | 0.08.08.238469v1.full.p |
| | | | | | | | | | | df) |
| Nb6-tri | ND | ND | ND | ND | ND | | ND | | ND | Michael Schoof et al., |
| | | | | | | | | | | 2020 |
| | | | | | | | | | | (https://www.biorxiv.or |
| | | | | | | | | | | g/content/10.1101/202 |
| | | | | | | | | | | 0.08.08.238469v1.full.p |
| | | | | | | | | | | df) |
| NIH- | N/A | IGHV3-3 | IGHJ7 | N/A | N/A | 3114 | AAVPSTYYNG | | N/A | Thomas Esparza et al., |
| CoVnb- | | (Alpaca) | (Alpaca) | | | | SYYYTCHPGG | | | 2020 |
| 101 | | | | | | | MDY | | | (https://www.biorxiv.or |
| | | | | | | | | | | g/content/10.1101/202 |
| | | | | | | | | | | 0.07.24.219857v1) |
| NIH- | N/A | IGHV3-3 | IGHJ7 | N/A | N/A | 3115 | AAVPSTYYSG | | N/A | Thomas Esparza et al., |
| CoVnb- | | (Alpaca) | (Alpaca) | | | | TYYYNCHPGG | | | 2020 |
| 102 | | | | | | | MDY | | | (https://www.biorxiv.or |
| | | | | | | | | | | g/content/10.1101/202 |
| | | | | | | | | | | 0.07.24.219857v1) |
| NIH- | N/A | IGHV3-3 | IGHJ7 | N/A | N/A | 3116 | ATAPGTYYKG | | N/A | Thomas Esparza et al., |
| CoVnb- | | (Alpaca) | (Alpaca) | | | | SYYPMCHYYG | | | 2020 |
| 103 | | | | | | | MDY | | | (https://www.biorxiv.or |
| | | | | | | | | | | g/content/10.1101/202 |
| | | | | | | | | | | 0.07.24.219857v1) |
| NIH- | N/A | IGHV3-3 | IGHJ7 | N/A | N/A | 3117 | ATRPLTYYSGS | | N/A | Thomas Esparza et al., |
| CoVnb- | | (Alpaca) | (Alpaca) | | | | YYTTCSDYGM | | | 2020 |
| 104 | | | | | | | DY | | | (https://www.biorxiv.or |
| | | | | | | | | | | g/content/10.1101/202 |
| | | | | | | | | | | 0.07.24.219857v1) |
| NIH | N/A | IGHV3-3 | IGHJ7 | N/A | N/A | 3118 | AAVPSTYYSG | | N/A | Thomas Esparza et al., |
| CoVnb- | | (Alpaca) | (Alpaca) | | | | SYYYTCHPGG | | | 2020 |
| 105 | | | | | | | MDY | | | (https://www.biorxiv.or |
| | | | | | | | | | | g/content/10.1101/202 |
| | | | | | | | | | | 0.07.24.219857v1) |
| NIH- | N/A | IGHV3-3 | IGHJ7 | N/A | N/A | 3119 | AAVPSTYYSG | | N/A | Thomas Esparza et al., |
| CoVnb- | | (Alpaca) | (Alpaca) | | | | SYYYTCHPGG | | | 2020 |
| 106 | | | | | | | MDY | | | (https://www.biorxiv.or |
| | | | | | | | | | | g/content/10.1101/202 |
| | | | | | | | | | | 0.07.24.219857v1) |
| NIH- | N/A | IGHV3-3 | IGHJ7 | N/A | N/A | 3120 | AAVPSTYYSG | | N/A | Thomas Esparza et al., |
| CoVnb- | | (Alpaca) | (Alpaca) | | | | SYYYTCHPGG | | | 2020 |
| 107 | | | | | | | IMDY | | | (https://www.biorxiv.or |
| | | | | | | | | | | g/content/10.1101/202 |
| | | | | | | | | | | 0.07.24.219857v1) |
| NIH- | N/A | IGHV3-3 | IGHJ7 | N/A | N/A | 3121 | AAVPSTYYSG | | N/A | Thomas Esparza et al., |
| CoVnb- | | (Alpaca) | (Alpaca) | | | | SYYYTCHPGG | | | 2020 |
| 108 | | | | | | | MDY | | | (https://www.biorxiv.or |
| | | | | | | | | | | g/content/10.1101/202 |
| | | | | | | | | | | 0.07.24.219857v1) |
| NIH- | N/A | IGHV3-3 | IGHJ7 | N/A | N/A | 3122 | ASFPSTYYSGS | | N/A | Thomas Esparza et al., |
| CoVnb- | | (Alpaca) | (Alpaca) | | | | YYYTCHPGG | | | 2020 |
| 109 | | | | | | | MDY | | | (https://www.biorxiv.or |
| | | | | | | | | | | g/content/10.1101/202 |
| | | | | | | | | | | 0.07.24.219857v1) |
| NIH- | N/A | IGHV3-3 | IGHJ7 | N/A | N/A | 3123 | AAVPSTYYSG | | N/A | Thomas Esparza et al., |
| CoVnb- | | (Alpaca) | (Alpaca) | | | | TYYYNCHPGA | | | 2020 |
| 110 | | | | | | | MHY | | | (https://www.biorxiv.or |
| | | | | | | | | | | g/content/10.1101/202 |
| | | | | | | | | | | 0.07.24.219857v1) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| NIH- | Nb | SARS-CoV2 | | | | S; RBD | Immunised Llama | 1010 | DVQLQESGGGSVEAGGSLRLSCAASGV | |
| CoVnb- | | | | | | | | | TLDYYAIGWFRQAPGKEREGVSCISSSD | |
| 111 | | | | | | | | | GSTYYADSVKGRFTTSRDNAKNTVYLQ | |
| | | | | | | | | | MNSLKPEDTADYYCAAVPSTYYSGTYYY | |
| | | | | | | | | | NCHPGAMHYWGKGTQVTVSS | |
| NIH- | Nb | SARS-CoV2 | | | | S; RBD | Immunised Llama | 1011 | DVQLQESGGGLVQPGGSLRLSCAASGL | |
| CoVnb- | | | | | | | | | TLDYYAIGWFRQAPGKEREGVSCISSSD | |
| 112 | | | | | | | | | GSTYYADSVKGRFTTSRDNAKNTVYLQ | |
| | | | | | | | | | MNSLKPEDTAVYYCAAVPSTYYSGTYYY | |
| | | | | | | | | | TCHPGGMDYWGKGTQVTVSS | |
| NIH- | Nb | SARS-CoV2 | | | | S; RBD | Immunised Llama | 1012 | DVQLQESGGGLVQPGGSLRLSCAASGL | |
| CoVnb- | | | | | | | | | TLDYYAIGWFRQAPGKEREGVSCISSSD | |
| 113 | | | | | | | | | GSTYYADSVKGRFTTSRDNAKNTVYLQ | |
| | | | | | | | | | MNSLKPEDTAVYYCAAVPSTYYSGTYYY | |
| | | | | | | | | | TCHPGGMDYWGKGTLVTVSS | |
| P2B-2F6 | Ab | SARS-CoV2 | SARS- | SARS-CoV2 | | S; RBD | B-cells; SARS-CoV2 | 1013 | QVQLQESGPGLVKPSETLSLTCTVSGYSI | 2055 |
| | | | CoV1 | | | | Human Patient | | SSGYYWGWIRQPPGKGLEWIGSIYHSG | |
| | | | | | | | | | STYYNPSLKTRVTISVDTSKNQFSLKLSSV | |
| | | | | | | | | | TAADTAVYYCARAVVGIVVVPAAGRRAF | |
| | | | | | | | | | DIWGQGTMVTVSS | |
| S110 | Ab | SARS-CoV1, | | SARS-Cov1 | | S; | B-cells; SARS-CoV1 | | ND | |
| | | SARS-CoV2 | | | | RBD + non- | Human Patient | | | |
| | | | | | | RBD | | | | |
| S124 | Ab | SARS-CoV1, | | SARS-CoV1 | | S; RBD | B-cells; SARS-CoV1 | | ND | |
| | | SARS-CoV2 | | | | | Human Patient | | | |
| S303 | Ab | SARS-CoV1, | | SARS-CoV1 | | S; RBD | B-cells; SARS-CoV1 | | ND | |
| | | SARS-CoV2 | | | | | Human Patient | | | |
| S304 | Ab | SARS-CoV1, | | SARS-CoV1, | | S; RBD | B-cells; SARS-CoV1 | | ND | |
| | | SARS-CoV2 | | SARS-CoV2 | | | Human Patient | | | |
| | | | | (weak) | | | | | | |
| S306 | Ab | SARS-CoV1, | | SARS-CoV1 | SARS-CoV2 | S; non- | B-cells; SARS-CoV1 | | ND | |
| | | SARS-CoV2 | | | | RBD | Human Patient | | | |
| S309 | Ab | SARS-CoV1, | | SARS-CoV2 | | S; RBD | B-cells; SARS-CoV1 | 1014 | QVQLVQSGAEVKKPGASVKVSCKASGY | 2056 |
| | | SARS-CoV2 | | and SARS- | | | Human Patient | | PFTSYGISWVRQAPGQGLEWMGWIST | |
| | | | | CoV1 | | | | | YNGNTNYAQKFQGRVTMTTDTSTTTGY | |
| | | | | | | | | | MELRRLRSDDTAVYYCARDYTRGAWFG | |
| | | | | | | | | | ESLIGGFDNWGQGTLVTVSS | |
| S310 | Ab | SARS-CoV1, | | SARS-CoV1 | | S; non- | B-cells; SARS-CoV1 | | ND | |
| | | SARS-CoV2 | | | | RBD | Human Patient | | | |
| S315 | Ab | SARS-CoV1, | | SARS-CoV2 | | S; RBD | B-cells; SARS-CoV1 | | ND | |
| | | SARS-CoV2 | | and SARS- | | | Human Patient | | | |
| Sb#1 | Nb | SARS-CoV2 | | | | S; RBD | Phage Display | 1015 | QVQLVESGGGLVQAGGSLRLSCAASGF | |
| | | | | | | | Library (Nanobody, | | PVRKANMHWYRQAPGKEREWVAAIM | |
| | | | | | | | non-immune) | | SKGEQTVYADSVEGRFTISRDNAKNTVY | |
| | | | | | | | | | LQMNSLKPEDTAVYYCRVFVGWHYFG | |
| | | | | | | | | | QGTQVTVS | |
| Sb#10 | Nb | SARS-CoV2 | | | | S; RBD | Phage Display | 1016 | QVQLVESGGGLVQAGGSLRLSCAASGF | |
| | | | | | | | Library (Nanobody, | | PVQSHYMRWYRQAPGKEREWVAAIES | |
| | | | | | | | non-immune) | | TGHHTAYADSVKGRFTISRDNAKNTVYL | |
| | | | | | | | | | QMNSLKPEDTAVYYCTVYVGYEYHGQG | |
| | | | | | | | | | TQVTVS | |
| Sb#11 | Nb | SARS-CoV2 | | | | S; RBD | Phage Display | 1017 | QVQLVESGGGLVQAGGSLRLSCAASGF | |
| | | | | | | | Library (Nanobody, | | PVETENMHWYRQAPGKEREWVAAIYS | |
| | | | | | | | non-immune) | | HGMWTAYADSVKGRFTISRDNTKNTVY | |
| | | | | | | | | | LQMNSLKPEDTAVYYCEVEVGKWYFGQ | |
| | | | | | | | | | GTQVTVS | |
| Sb#12 | Nb | SARS-CoV2 | | | | S; RBD | Phage Display | 1018 | QVQLVESGGGLVQAGGSLRLSCAASGF | |
| | | | | | | | Library (Nanobody, | | PVKASRMYWYRQAPGKEREWVAAIQS | |
| | | | | | | | non-immune) | | FGEVTWYADSVKGRFTISRDNAKNTVYL | |
| | | | | | | | | | QMNSLKPEDTAVYYCYVWVGQEYWG | |
| | | | | | | | | | QGTQVTVS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| NIH- | N/A | IGHV3-3 | IGHJ7 | N/A | N/A | 3124 | AAVPSTYYSG | | N/A | Thomas Esparza et al., |
| CoVnb- | | (Alpaca) | (Alpaca) | | | | TYYYNCHPGA | | | 2020 |
| 111 | | | | | | | MHY | | | (https://www.biorxiv.or |
| | | | | | | | | | | g/content/10.1101/202 |
| | | | | | | | | | | (0.07.24.219857v1) |
| NIH- | N/A | IGHV3-3 | IGHJ7 | N/A | N/A | 3125 | AAVPSTYYSG | | N/A | Thomas Esparza et al., |
| CoVnb- | | (Alpaca) | (Alpaca) | | | | TYYYTCHPGG | | | 2020 |
| 112 | | | | | | | MDY | | | (https://www.biorxiv.or |
| | | | | | | | | | | g/content/10.1101/202 |
| | | | | | | | | | | 0.07.24.219857v1) |
| NIH- | N/A | IGHV3-3 | IGHJ7 | N/A | N/A | 3126 | AAVPSTYYSG | | N/A | Thomas Esparza et al., |
| CoVnb- | | (Alpaca) | (Alpaca) | | | | TYYYTCHPGG | | | 2020 |
| 113 | | | | | | | MDY | | | (https://www.biorxiv.or |
| | | | | | | | | | | g/content/10.1101/202 |
| | | | | | | | | | | 0.07.24.219857v1) |
| P2B-2F6 | QSALTQPPSASGSPGQSVTISCTGTSS | IGHV4-38- | IGHJ3 | IGLV2-8 | IGLJ3 | 3127 | ARAVVGIVVV | 4292 | SSYAGSNN | Bin Ju et al., 2020 |
| | DVGGYNYVSWYQQHPGKAPKLMIYE | 2 | (Human) | (Human) | (Human) | | PAAGRRAFDI | | LV | (https://www.nature.co |
| | VSKRPSGVPDRFSGSKSGNTASLTVSG | (Human) | | | | | | | | m/articles/s41586-020- |
| | LQAEDEADYYCSSYAGSNNLVFGGGT | | | | | | | | | 2380-z) |
| | KLTVL | | | | | | | | | |
| S110 | ND | IGHV3-30 | ND | IGKV2-30 | ND | 3128 | AKDRFQFARS | 4293 | MQGTHW | Dora Pinto et al., 2020 |
| | | (Human) | | (Human) | | | WYGDYFDY | | PPT | (https://www.nature.co |
| | | | | | | | | | | m/articles/s41586-020- |
| | | | | | | | | | | 2349-y) |
| S124 | ND | IGHV2-26 | ND | IGKV1-39 | ND | 3129 | ARINTAAYDY | 4294 | QQSYSTPP | Dora Pinto et al., 2020 |
| | | (Human) | | (Human) | | | DSTTFDI | | T | (https://www.nature.co |
| | | | | | | | | | | m/articles/s41586-020- |
| | | | | | | | | | | 2349-y) |
| S303 | ND | IGHV3-23 | ND | IGKV1-5 | ND | 3130 | ARERDDIFPM | 4295 | QQYDTYS | Dora Pinto et al., 2020 |
| | | (Human) | | (Human) | | | GLNAFDI | | WT | (https://www.nature.co |
| | | | | | | | | | | m/articles/s41586-020- |
| | | | | | | | | | | 2349-y) |
| S304 | ND | IGHV3-13 | ND | IGKV1-39 | ND | 3131 | ARGDSSGYYY | 4296 | QQSYVSPT | Dora Pinto et al., 2020 |
| | | (Human) | | (Human) | | | YFDY | | YT | (https://www.nature.co |
| | | | | | | | | | | m/articles/s41586-020- |
| | | | | | | | | | | 2349-y) |
| S306 | ND | IGHV1-18 | ND | IGKV3-11 | ND | 3132 | ASDYFDSSGY | 4297 | QQRSNWP | Dora Pinto et al., 2020 |
| | | (Human) | | (Human) | | | YHSFDY | | PGCS | (https://www.nature.co |
| | | | | | | | | | | m/articles/s41586-020- |
| | | | | | | | | | | 2349-y) |
| S309 | EIVLTQSPGTLSLSPGERATLSCRASQT | IGHV1-18 | IGHJ4 | IGKV3-20 | IGKJ4 | 3133 | ARDYTRGAW | 4298 | QQHDTSLT | Dora Pinto et al., 2020 |
| | VSSTSLAWYQQKPGQAPRLLIYGASSR | (Human) | (Human) | (Human) | (Human) | | FGESLIGGFD | | | (https://www.nature.co |
| | ATGIPDRFSGSGSGTDFTLTISRLEPED | | | | | | N | | | m/articles/s41586-020- |
| | FAVYYCQQHDTSLTFGGGTKVEIK | | | | | | | | | 2349-y) |
| S310 | ND | IGHV1-69 | ND | IGLV2-23 | ND | 3134 | ATRTYDSSGY | 4299 | CSYAGSDT | Dora Pinto et al., 2020 |
| | | (Human) | | (Human) | | | RPYYYGLDV | | VI | (https://www.nature.co |
| | | | | | | | | | | m/articles/s41586-020- |
| | | | | | | | | | | 2349-y) |
| S315 | ND | IGHV3-7 | ND | IGLV3-25 | ND | 3135 | ARDLWWND | 4300 | QSADSSGT | Dora Pinto et al., 2020 |
| | | (Human) | | (Human) | | | QAHYYGMDV | | V | (https://www.nature.co |
| | | | | | | | | | | m/articles/s41586-020- |
| | | | | | | | | | | 2349-y) |
| Sb#1 | N/A | IGHV3-3 | IGHJ4 | N/A | N/A | 3136 | RVFVGWHY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |
| Sb#10 | N/A | IGHV3-3 | IGHJ4 | N/A | N/A | 3137 | TVYVGYEY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |
| Sb#11 | N/A | IGHV3-3 | IGHJ4 | N/A | N/A | 3138 | EVEVGKWY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |
| Sb#12 | N/A | IGHV3-3 | IGHJ5 | N/A | N/A | 3139 | YVWVGQEY | | N/A | 2020 |
| | | (Alpaca) | (Alpaca) | | | | | | | Justin Walter et al., |
| | | | | | | | | | | (https://www.biorxiv.or |
| | | | | | | | | | | g/content/10.1101/202 |
| | | | | | | | | | | 0.04.16.045419v2) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| Sb#13 | Nb | SARS-CoV2 | | | | S; RBD | Phage Display | 1019 | QVQLVESGGGLVQAGGSLRLSCAASGF | |
| | | | | | | | Library (Nanobody, | | PVYASNMHWYRQAPGKEREWVAAIES | |
| | | | | | | | non-immune) | | QGYMTAYADSVKGRFTISRDNAKNTVY | |
| | | | | | | | | | LQMNSLKPEDTAVYYCWVIVGEYYVGQ | |
| | | | | | | | | | GTQVTVS | |
| Sb#14 | Nb | SARS-CoV2 | | | | S; RBD | Phage Display | 1020 | QVQLVESGGGLVQAGGSLRLSCAASGF | |
| | | | | | | | Library (Nanobody, | | PVQAREMEWYRQAPGKEREWVAAIKS | |
| | | | | | | | non-immune) | | TGTYTAYAYSVKGRFTISRDNAKNTVYLQ | |
| | | | | | | | | | MNSLKPEDTAVYYCYVYVGSSYIGQGTQ | |
| | | | | | | | | | VTVS | |
| Sb#15 | Nb | SARS-CoV2 | | | | S; RBD | Phage Display | 1021 | QVQLVESGGGLVQAGGSLRLSCAASGF | |
| | | | | | | | Library (Nanobody, | | PVKNFEMEWYRKAPGKEREWVAAIQS | |
| | | | | | | | non-immune) | | GGVETYYADSVKGRFTISRDNAKNTVYL | |
| | | | | | | | | | QMNSLKPEDTAVYYCFVYVGRSYIGQGT | |
| | | | | | | | | | QVTVS | |
| Sb#16 | Nb | SARS-CoV2 | | | | S; RBD | Phage Display | 1022 | QVQLVESGGGLVQAGGSLRLSCAASGF | |
| | | | | | | | Library (Nanobody, | | PVAYKTMWWYRQAPGKEREWVAAIES | |
| | | | | | | | non-immune) | | YGIKWTRYADSVKGRFTISRDNAKNTVY | |
| | | | | | | | | | LQMNSLKPEDTAVYYCIVWVGAQYHG | |
| | | | | | | | | | QGTQVTVS | |
| Sb#17 | Nb | SARS-CoV2 | | | | S; RBD | Phage Display | 1023 | QVQLVESGGGLVQAGGSLRLSCAASGF | |
| | | | | | | | Library (Nanobody, | | PVAGRNMWWYRQAPGKEREWVAAIY | |
| | | | | | | | non-immune) | | SSGTYTEYADSVKGRFTISRDNAKNTVYL | |
| | | | | | | | | | QMNSLKPEDTAVYYCHVWVGSLYKGQ | |
| | | | | | | | | | GTQVTVS | |
| Sb#18 | Nb | SARS-CoV2 | | | | S; RBD | Phage Display | 1024 | QVQLVESGGGLVQAGGSLRLSCAASGF | |
| | | | | | | | Library (Nanobody, | | PVKHARMWWYRQAPGKEREWVAAID | |
| | | | | | | | non-immune) | | SHGDTTWYADSVKGRFTISRDNAKNTV | |
| | | | | | | | | | YLQMNSLKPEDTAVYYCYVYVGASYWG | |
| | | | | | | | | | QGTQVTVS | |
| Sb#19 | Nb | SARS-CoV2 | | | | S; RBD | Phage Display | 1025 | QVQLVESGGGLVQAGGSLRLSCAASGF | |
| | | | | | | | Library (Nanobody, | | PVNSHEMTWYRQAPGKEREWVAAIQS | |
| | | | | | | | non-immune) | | TGTVTEYADSVKGRFTISRDNAKNTVYL | |
| | | | | | | | | | QMNSLKPEDTAVYYCYVYVGSSYLGQG | |
| | | | | | | | | | TQVTVS | |
| Sb#2 | Nb | SARS-CoV2 | | | | S; RBD | Phage Display | 1026 | QVQLVESGGGLVQAGGSLRLSCATSGF | |
| | | | | | | | Library (Nanobody, | | PVYQANMHWYRQAPGKEREWVAAIQ | |
| | | | | | | | non-immune) | | SYGDGTHYADSVKGRFTISRDNAKNTVY | |
| | | | | | | | | | LQMNSLKPEDTAVYYCRAVYVGMHYFG | |
| | | | | | | | | | QGTQVTVS | |
| Sb#20 | Nb | SARS-CoV2 | | | | S; RBD | Phage Display | 1027 | QVQLVESGGGLVQAGGSLRLSCAASGF | |
| | | | | | | | Library (Nanobody, | | PVEQREMEWYRQAPGKEREWVAAIDS | |
| | | | | | | | non-immune) | | NGNYTFYADSVKGRFTISRDNAKNTVYL | |
| | | | | | | | | | QMNSLKPEDTAVYYCYVYVGKSYIGQGT | |
| | | | | | | | | | QVTVS | |
| Sb#21 | Nb | SARS-CoV2 | | | | S; RBD | Phage Display | 1028 | QVQLVESGGGLVQAGGSLRLSCAASGF | |
| | | | | | | | Library (Nanobody, | | PVKHHWMFWYRQAPGKEREWVAAIK | |
| | | | | | | | non-immune) | | SYGYGTEYADSVKGRFTISRDNAKNTVYL | |
| | | | | | | | | | QMNSLKPEDTAVYYCFVGVGTHYAGQ | |
| | | | | | | | | | GTQVTVS | |
| Sb#22 | Nb | SARS-CoV2 | | | | S; RBD | Phage Display | 1029 | QVQLVESGGGLVQAGGSLRLSCAASGF | |
| | | | | | | | Library (Nanobody, | | PVYAAEMEWYRQAPGKEREWVAAISS | |
| | | | | | | | non-immune) | | QGTITYYADSVKGRFTISRDNAKNTVYLQ | |
| | | | | | | | | | MNSLKPEDTAVYYCFVYVGKSYIGQGTQ | |
| | | | | | | | | | VSVS | |
| Sb#23 | Nb | SARS-CoV2 | | | | S; RBD | Phage Display | 1030 | QVQLVESGGGLVQAGGSLRLSCAASGF | |
| | | | | | | | Library (Nanobody, | | PVYAAEMEWYRQAPGKEREWVAAISS | |
| | | | | | | | non-immune) | | QGTITYYADSVKGRFTISRDNAKNTVYLQ | |
| | | | | | | | | | MNSLKPEDTAVYYCFVYVGKSYIGQGTQ | |
| | | | | | | | | | VSVS | |
| Sb#25 | Nb | SARS-CoV2 | | | | S; RBD | Phage Display | 1031 | QVQLVESGGGLVQAGGSLRLSCAASGF | |
| | | | | | | | Library (Nanobody, | | PVHAWEMAWYRQAPGKEREWVAAIR | |
| | | | | | | | non-immune) | | SFGSSTHYADSVKGRFTISRDNAKNTVYL | |
| | | | | | | | | | QMNSLKPEDTAVYYCNVKDFGTHHYAY | |
| | | | | | | | | | DYWGQGTQVTVS | |
| Sb#26 | Nb | SARS-CoV2 | | | | S; RBD | Phage Display | 1032 | QVQLVESGGGLVQAGGSLRLSCAASGF | |
| | | | | | | | Library (Nanobody, | | PVNTWWMHWYRQAPGKEREWVAAIT | |
| | | | | | | | non-immune) | | SWGFRTYYADSVKGRFTISRDNAKNTVY | |
| | | | | | | | | | LQMNSLKPEDTAVYYCNVKDKGMAVQ | |
| | | | | | | | | | WYDYWGQGTQVTVS | |
| Sb#27 | Nb | SARS-CoV2 | | | | S; RBD | Phage Display | 1033 | QVQLVESGGGLVQAGGSLRLSCAASGF | |
| | | | | | | | Library (Nanobody, | | PVYNTWMEWYRQAPGKEREWVAAITS | |
| | | | | | | | non-immune) | | HGYKTYYADSVKGRFTISRDNAKNTVYL | |
| | | | | | | | | | QMNSLKPEDTAVYYCNVKDEGDMFTA | |
| | | | | | | | | | YDYWGQGTQVTVS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| Sb#13 | N/A | IGHV3-3 | IGHJ4 | N/A | N/A | 3140 | WVIVGEYY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |
| Sb#14 | N/A | IGHV3-3 | IGHJ4 | N/A | N/A | 3141 | YVYVGSSY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |
| Sb#15 | N/A | IGHV3-3 | IGHJ4 | N/A | N/A | 3142 | FVYVGRSY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |
| Sb#16 | N/A | IGHV3S5 | IGHJ4 | N/A | N/A | 3143 | IVWVGAQY | | N/A | Justin Walter et al., |
| | | 3 | (Alpaca) | | | | | | | 2020 |
| | | (Alpaca) | | | | | | | | (https://www.biorxiv.or |
| | | | | | | | | | | g/content/10.1101/202 |
| | | | | | | | | | | 0.04.16.045419v2) |
| Sb#17 | N/A | IGHV3-3 | IGHJ4 | N/A | N/A | 3144 | HVWVGSLY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |
| Sb#18 | N/A | IGHV3-3 | IGHJ4 | N/A | N/A | 3145 | YVYVGASY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |
| Sb#19 | N/A | IGHV3-3 | IGHJ4 | N/A | N/A | 3146 | YVYVGSSY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |
| Sb#2 | N/A | IGHV3-3 | IGHJ4 | N/A | N/A | 3147 | RAVYVGMHY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |
| Sb#20 | N/A | IGHV3-3 | IGHJ4 | N/A | N/A | 3148 | YVYVGKSY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |
| Sb#21 | N/A | IGHV3-3 | IGHJ4 | N/A | N/A | 3149 | FVGVGTHY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |
| Sb#22 | N/A | IGHV3-3 | IGHJ4 | N/A | N/A | 3150 | FVYVGKSY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |
| Sb#23 | N/A | IGHV3-3 | IGHJ4 | N/A | N/A | 3151 | FVYVGKSY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |
| Sb#25 | N/A | IGHV3S5 | IGHJ4 | N/A | N/A | 3152 | NVKDFGTHH | | N/A | Justin Walter et al., |
| | | 3 | (Alpaca) | | | | YAYDY | | | 2020 |
| | | (Alpaca) | | | | | | | | (https://www.biorxiv.or |
| | | | | | | | | | | g/content/10.1101/202 |
| | | | | | | | | | | 0.04.16.045419v2) |
| Sb#26 | N/A | IGHV3S5 | IGHJ4 | N/A | N/A | 3153 | NVKDKGMAV | | N/A | Justin Walter et al., |
| | | 3 | (Alpaca) | | | | QWYDY | | | 2020 |
| | | (Alpaca) | | | | | | | | (https://www.biorxiv.or |
| | | | | | | | | | | g/content/10.1101/202 |
| | | | | | | | | | | 0.04.16.045419v2) |
| Sb#27 | N/A | IGHV3S5 | IGHJ4 | N/A | N/A | 3154 | NVKDEGDMF | | N/A | Justin Walter et al., |
| | | 3 | (Alpaca) | | | | TAYDY | | | 2020 |
| | | (Alpaca) | | | | | | | | (https://www.biorxiv.or |
| | | | | | | | | | | g/content/10.1101/202 |
| | | | | | | | | | | 0.04.16.045419v2) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| Sb#28 | Nb | SARS-CoV2 | | | | S; RBD | Phage Display | 1034 | QVQLVESGGGLVQAGGSLRLSCAASGF | |
| | | | | | | | Library (Nanobody, | | PVYHSTMFWYRQAPGKEREWVAAIYSS | |
| | | | | | | | non-immune) | | GQHTYYADSVKGRFTISRDNAKNTVYLQ | |
| | | | | | | | | | MNSLKPEDTAVYYCNVKDSGQWRQEY | |
| | | | | | | | | | DYWGQGTQVTVS | |
| Sb#29 | Nb | SARS-CoV2 | | | | S; RBD | Phage Display | 1035 | QVQLVESGGGLVQAGGSLRLSCAASGF | |
| | | | | | | | Library (Nanobody, | | PVEHEMAWYRQAPGKEREWVAAIRS | |
| | | | | | | | non-immune) | | MGRKTLYADSVKGRFTISRDNAKNTVYL | |
| | | | | | | | | | QMNSLKPEDTAVYYCNVKDFGYTWHEY | |
| | | | | | | | | | DYWGQGTQVTVS | |
| Sb#3 | Nb | SARS-CoV2 | | | | S; RBD | Phage Display | 1036 | QVQLVESGGGLVQAGGSLRLSCAASGF | |
| | | | | | | | Library (Nanobody, | | PVNYKTMWWYRQAPGKEREWVAAIW | |
| | | | | | | | non-immune) | | SYGHTTHYADSVKGRFTISRDNAKNTVY | |
| | | | | | | | | | LQMNSLKPEDTAVYYCVVWVGHNYEG | |
| | | | | | | | | | QGTQVTVS | |
| Sb#30 | Nb | SARS-CoV2 | | | | S; RBD | Phage Display | 1037 | QVQLVESGGGLVQAGGSLRLSCAASGF | |
| | | | | | | | Library (Nanobody, | | PVTMAWMWWYRQAPGKEREWVAAI | |
| | | | | | | | non-immune) | | RSEGVRTYYADSVKGRFTISRDNAKNTV | |
| | | | | | | | | | YLQMNSLKPEDTAVYYCNVKDYGQAHA | |
| | | | | | | | | | YYDYWGQGTQVTVS | |
| Sb#31 | Nb | SARS-CoV2 | | | | S; RBD | Phage Display | 1038 | QVQLVESGGGLVQAGGSLRLSCAASGF | |
| | | | | | | | Library (Nanobody, | | PVNSHFMEWYRQAPGKEREWVAAIQH | |
| | | | | | | | non-immune) | | SSGFHTYYADSVKGRFTISRDNAKNTVYL | |
| | | | | | | | | | QMNSLKPEDTAVYYCNVKDTGTTEDYD | |
| | | | | | | | | | YWGQGTQVTVS | |
| Sb#32 | Nb | SARS-CoV2 | | | | S; RBD | Phage Display | 1039 | QVQLDESGGGLVQAGGSLRLSCAASGF | |
| | | | | | | | Library (Nanobody, | | PVYHAWMEWYRQAPGKEREWVAAITS | |
| | | | | | | | non-immune) | | SGRHTYYADSVKGRFTISRDNAKNTVYL | |
| | | | | | | | | | QMNSLKPEDTAVYYCNVKDAGRVYNSY | |
| | | | | | | | | | DYWGQGTQVTVS | |
| Sb#33 | Nb | SARS-CoV2 | | | | S; RBD | Phage Display | 1040 | QVQLVESGGGLVQAGGSLRLSCAASGF | |
| | | | | | | | Library (Nanobody, | | PVAHAWMEWYRQAPGKEREWVAAIT | |
| | | | | | | | non-immune) | | SYGYKTYYADSVKGRFTISRDNAKNTVYL | |
| | | | | | | | | | QMNSLKPEDTAVYYCNVKDTGTYRFYY | |
| | | | | | | | | | DYWGQGTQVTVS | |
| Sb#34 | Nb | SARS-CoV2 | | | | S; RBD | Phage Display | 1041 | QVQLVESGGGLVQAGGSLRLSCAASGF | |
| | | | | | | | Library (Nanobody, | | PVWNQTMVWYRQAPGKEREWVAAI | |
| | | | | | | | non-immune) | | WSMGHTYYADSVKGRFTISRDNAKNTV | |
| | | | | | | | | | YLQMNSLKPEDTAVYYCNVKDAGVYNR | |
| | | | | | | | | | YYDYWGQGTQVTVS | |
| Sb#35 | Nb | SARS-CoV2 | | | | S; RBD | Phage Display | 1042 | QVQLVESGGGLVQAGGSLRLSCAASGF | |
| | | | | | | | Library (Nanobody, | | PVEHYWMEWYRQAPGKEREWVAAITS | |
| | | | | | | | non-immune) | | FGYRTYYADSVKGRFTISRDNAKNTVYL | |
| | | | | | | | | | QMNSLKPEDTAVYYCNVKDWGFASHA | |
| | | | | | | | | | YDYWGQGIQVTVS | |
| Sb#36 | Nb | SARS-CoV2 | | | | S; RBD | Phage Display | 1043 | QVQLVESGGGLVQAGGSLRLSCAASGF | |
| | | | | | | | Library (Nanobody, | | PEIAWEMAWYRQAPGKEREWVAAIRS | |
| | | | | | | | non-immune) | | FGERTLYADSVKGRFTISRDNAKNTVYL | |
| | | | | | | | | | QMNSLKPEDTAVYYCNVKDFGWQHQE | |
| | | | | | | | | | YDYWGQGTQVTVS | |
| Sb#37 | Nb | SARS-CoV2 | | | | S; RBD | Phage Display | 1044 | QVQLVESGGGLVQAGGSLRLSCAASGF | |
| | | | | | | | Library (Nanobody, | | PVYHAYMEWYRQAPGKEREWVAAIYS | |
| | | | | | | | non-immune) | | NGEHTYYADSVKGRFTISRDNAKNTVYL | |
| | | | | | | | | | QMNSLKPEDTAVYYCNVKDSGSFNQAY | |
| | | | | | | | | | DYWGQGTQVTVS | |
| Sb#38 | Nb | SARS-CoV2 | | | | S; RBD | Phage Display | 1045 | QVQLVESGGGLVQAGGSLRLSCAASGF | |
| | | | | | | | Library (Nanobody, | | PVEWSHMHWYRQAPGKEREWVAAIV | |
| | | | | | | | non-immune) | | SKGGYTLYADSVKGRFTISRDNAKNTVYL | |
| | | | | | | | | | QMNSLKPEDTAVYYCNVKDYGVHFKRY | |
| | | | | | | | | | DYWGQGTQVTVI | |
| Sb#39 | Nb | SARS-CoV2 | | | | S; RBD | Phage Display | 1046 | QVQLVESGGGLVQAGGSLRLSCAASGF | |
| | | | | | | | Library (Nanobody, | | PVFHVWMEWYRQAPGKEREWVAAID | |
| | | | | | | | non-immune) | | SAGWHTYYADSVKGRFTISRDNAKNTV | |
| | | | | | | | | | YLQMNSLKPEDTAVYYCNVKDAGNTTS | |
| | | | | | | | | | AYDYWGQGTQVTVS | |
| Sb#4 | Nb | SARS-CoV2 | | | | S; RBD | Phage Display | 1047 | QVQLVESGGGLVQAGGSLRLSCAASGF | |
| | | | | | | | Library (Nanobody, | | PVYAQNMHWYRQAPGKEREWVAAIYS | |
| | | | | | | | non-immune) | | HGYWTLYADSVKGRFTISRDNAKNTVYL | |
| | | | | | | | | | QMNSLKPEDTAVYYCEVQVGAWYTGQ | |
| | | | | | | | | | GTQVTVS | |
| Sb#40 | Nb | SARS-CoV2 | | | | S; RBD | Phage Display | 1048 | QVQLVESGGGLVQAGGSLRLSCAASGF | |
| | | | | | | | Library (Nanobody, | | PVYYNWMEWYRQAPGKEREWVAAIH | |
| | | | | | | | non-immune) | | SNGDETFYADSVKGRFTISRDNAKNTVY | |
| | | | | | | | | | LQMNSLKPEDTAVYYCNVKDIDAEAYAY | |
| | | | | | | | | | DYWGQGTQVTVS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| Sb#28 | N/A | IGHV3-3 | IGHJ4 | N/A | N/A | 3155 | NVKDSGQWR | | N/A | Justin Walter et al., |
| | | (Alpaca) | (Alpaca) | | | | QEYDY | | | 2020 |
| | | | | | | | | | | (https://www.biorxiv.or |
| | | | | | | | | | | g/content/10.1101/202 |
| | | | | | | | | | | 0.04.16.045419v2) |
| Sb#29 | N/A | IGHV3S5 | IGHJ4 | N/A | N/A | 3156 | NVKDFGYTW | | N/A | Justin Walter et al., |
| | | 3 | (Alpaca) | | | | HEYDY | | | 2020 |
| | | (Alpaca) | | | | | | | | (https://www.biorxiv.or |
| | | | | | | | | | | g/content/10.1101/202 |
| | | | | | | | | | | 0.04.16.045419v2) |
| Sb#3 | N/A | IGHV3-3 | IGHJ4 | N/A | N/A | 3157 | VVWVGHNY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |
| Sb#30 | N/A | IGHV3-3 | IGHJ4 | N/A | N/A | 3158 | NVKDYGQAH | | N/A | Justin Walter et al., |
| | | (Alpaca) | (Alpaca) | | | | AYYDY | | | 2020 |
| | | | | | | | | | | (https://www.biorxiv.or |
| | | | | | | | | | | g/content/10.1101/202 |
| | | | | | | | | | | 0.04.16.045419v2) |
| Sb#31 | N/A | IGHV3-3 | IGHJ4 | N/A | N/A | 3159 | NVKDTGTTED | | N/A | Justin Walter et al., |
| | | (Alpaca) | (Alpaca) | | | | YDY | | | 2020 |
| | | | | | | | | | | (https://www.biorxiv.or |
| | | | | | | | | | | g/content/10.1101/202 |
| | | | | | | | | | | 0.04.16.045419v2) |
| Sb#32 | N/A | IGHV3-3 | IGHJ4 | N/A | N/A | 3160 | NVKDAGRVY | | N/A | Justin Walter et al., |
| | | (Alpaca) | (Alpaca) | | | | NSYDY | | | 2020 |
| | | | | | | | | | | (https://www.biorxiv.or |
| | | | | | | | | | | g/content/10.1101/202 |
| | | | | | | | | | | 0.04.16.045419v2) |
| Sb#33 | N/A | IGHV3S5 | IGHJ4 | N/A | N/A | 3161 | NVKDTGTYRF | | N/A | Justin Walter et al., |
| | | 3 | (Alpaca) | | | | YYDY | | | 2020 |
| | | (Alpaca) | | | | | | | | (https://www.biorxiv.or |
| | | | | | | | | | | g/content/10.1101/202 |
| | | | | | | | | | | 0.04.16.045419v2) |
| Sb#34 | N/A | IGHV3S5 | IGHJ4 | N/A | N/A | 3162 | NVKDAGVYN | | N/A | Justin Walter et al., |
| | | 3 | (Alpaca) | | | | RYYDY | | | 2020 |
| | | (Alpaca) | | | | | | | | (https://www.biorxiv.or |
| | | | | | | | | | | g/content/10.1101/202 |
| | | | | | | | | | | 0.04.16.045419v2) |
| Sb#35 | N/A | IGHV3-3 | IGHJ4 | N/A | N/A | 3163 | NVKDWGFAS | | N/A | Justin Walter et al., |
| | | (Alpaca) | (Alpaca) | | | | HAYDY | | | 2020 |
| | | | | | | | | | | (https://www.biorxiv.or |
| | | | | | | | | | | g/content/10.1101/202 |
| | | | | | | | | | | 0.04.16.045419v2) |
| Sb#36 | N/A | IGHV3S5 | IGHJ4 | N/A | N/A | 3164 | NVKDFGWQ | | N/A | Justin Walter et al., |
| | | 3 | (Alpaca) | | | | HQEYDY | | | 2020 |
| | | (Alpaca) | | | | | | | | (https://www.biorxiv.or |
| | | | | | | | | | | g/content/10.1101/202 |
| | | | | | | | | | | 0.04.16.045419v2) |
| Sb#37 | N/A | IGHV3-3 | IGHJ4 | N/A | N/A | 3165 | NVKDSGSFN | | N/A | Justin Walter et al., |
| | | (Alpaca) | (Alpaca) | | | | QAYDY | | | 2020 |
| | | | | | | | | | | (https://www.biorxiv.or |
| | | | | | | | | | | g/content/10.1101/202 |
| | | | | | | | | | | 0.04.16.045419v2) |
| Sb#38 | N/A | IGHV3S5 | IGHJ4 | N/A | N/A | 3166 | NVKDYGVHF | | N/A | Justin Walter et al., |
| | | 3 | (Alpaca) | | | | KRYDY | | | 2020 |
| | | (Alpaca) | | | | | | | | (https://www.biorxiv.or |
| | | | | | | | | | | g/content/10.1101/202 |
| | | | | | | | | | | 0.04.16.045419v2) |
| Sb#39 | N/A | IGHV3-3 | IGHJ4 | N/A | N/A | 3167 | NVKDAGNTT | | N/A | Justin Walter et al., |
| | | (Alpaca) | (Alpaca) | | | | SAYDY | | | 2020 |
| | | | | | | | | | | (https://www.biorxiv.or |
| | | | | | | | | | | g/content/10.1101/202 |
| | | | | | | | | | | 0.04.16.045419v2) |
| Sb#4 | N/A | IGHV3-3 | IGHJ4 | N/A | N/A | 3168 | EVQVGAWY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |
| Sb#40 | N/A | IGHV3S5 | IGHJ4 | N/A | N/A | 3169 | NVKDIDAEAY | | N/A | Justin Walter et al., |
| | | 3 | (Alpaca) | | | | AYDY | | | 2020 |
| | | (Alpaca) | | | | | | | | (https://www.biorxiv.or |
| | | | | | | | | | | g/content/10.1101/202 |
| | | | | | | | | | | 0.04.16.045419v2) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| Sb#41 | Nb | SARS-CoV2 | | | | S; RBD | Phage Display | 1049 | QVQLVESGGGLVQAGGSLRLSCAASGF | |
| | | | | | | | Library (Nanobody, | | PVYHVWMEWYRQAPGKEREWVAAITS | |
| | | | | | | | non-immune) | | SGSHTYYADSVKGRFTISRDNAKNTVYL | |
| | | | | | | | | | QMNSLKPEDTAVYYCNVKDSGQWRVQ | |
| | | | | | | | | | YDYWGQGTQVTVS | |
| Sb#42 | Nb | SARS-CoV2 | | | | S; RBD | Phage Display | 1050 | QVQLVESGGGLVQAGGSLRLSCAASGF | |
| | | | | | | | Library (Nanobody, | | PVYWHHMHWYRQAPGKEREWVAAIIS | |
| | | | | | | | non-immune) | | WGWYTTYADSVKGRFTISRDNAKNTVY | |
| | | | | | | | | | LQMNSLKPEDTAVYYCNVKDHGAQNQ | |
| | | | | | | | | | MYDYWGQGTQVTVS | |
| Sb#45 | Nb | SARS-CoV2 | | | | S; RBD | Phage Display | 1051 | QVQLVESGGGLVQAGGSLRLSCAASGF | |
| | | | | | | | Library (Nanobody, | | PVYRDRMAWYRQAPGKEREWVAAIYS | |
| | | | | | | | non-immune) | | AGQQTRYADSVKGRFTISRDNAKNTVYL | |
| | | | | | | | | | QMNSLKPEDTAVYYCNVKDVGHHYEYY | |
| | | | | | | | | | DYWGQGTQVTVS | |
| Sb#46 | Nb | SARS-CoV2 | | | | S; RBD | Phage Display | 1052 | QVQLVESGGGLVQAGGSLRLSCAASGF | |
| | | | | | | | Library (Nanobody, | | PVDNGYMHWYRQAPGKEREWVAAID | |
| | | | | | | | non-immune) | | SYGWHTIYADSVKGRFTISRDNAKNTVY | |
| | | | | | | | | | LQMNSLKPEDTAVYYCNVKDKGQMRA | |
| | | | | | | | | | AYDYWGQGTQVTVS | |
| Sb#47 | Nb | SARS-CoV2 | | | | S; RBD | Phage Display | 1053 | QVQLVESGGGLVQAGGSLRLSCAASGF | |
| | | | | | | | Library (Nanobody, | | PVSWHSMYWYRQAPGKEREWVAAIFS | |
| | | | | | | | non-immune) | | EGDWTYYADSVKGRFTISRDNAKNTVYL | |
| | | | | | | | | | QMNSLKPEDTAVYYCNVKDYGSSYYKY | |
| | | | | | | | | | DYWGQGTQVTVS | |
| Sb#48 | Nb | SARS-CoV2 | | | | S; RBD | Phage Display | 1054 | QVQLVESGGGLVQAGGSLRLSCAASGF | |
| | | | | | | | Library (Nanobody, | | PVSQSVMAWYRQAPGKEREWVAAIYS | |
| | | | | | | | non-immune) | | KGQYTHYADSVKGRFTISRDNAKNTVYL | |
| | | | | | | | | | QMNSLKPEDTAVYYCNVKDAGSSYWD | |
| | | | | | | | | | YDYWGQGTQVTVS | |
| Sb#49 | Nb | SARS-CoV2 | | | | S; RBD | Phage Display | 1055 | QVQLVESGGGSVQAGGSLRLSCAASGSI | |
| | | | | | | | Library (Nanobody, | | GQIEYLGWFRQAPGKEREGVAALNTW | |
| | | | | | | | non-immune) | | TGRTYYADSVKGRFTVSLDNAKNTVYLQ | |
| | | | | | | | | | MNSLKPEDTALYYCAAARWGRTKPLNT | |
| | | | | | | | | | YYYSYWGQGTPVTVS | |
| Sb#5 | Nb | SARS-CoV2 | | | | S; RBD | Phage Display | 1056 | QVQLVESGGGLVQAGGSLRLSCAASGF | |
| | | | | | | | Library (Nanobody, | | PVFSGHMHWYRQAPGKEREWVAAILS | |
| | | | | | | | non-immune) | | NGDSTHYADSVKGRFTISRDNAKNTVYL | |
| | | | | | | | | | QMNSLKPEDTAVYYCRVHVGAHYFGQ | |
| | | | | | | | | | GTQVTVS | |
| Sb#50 | Nb | SARS-CoV2 | | | | S; RBD | Phage Display | 1057 | QVQLVESGGGSVQAGGSLRLSCAASGYI | |
| | | | | | | | Library (Nanobody, | | DKIVYLGWFRQAPGKEREGVAALYTLSG | |
| | | | | | | | non-immune) | | HTYYADSVKGRFTVSLDNAKNTVYLQM | |
| | | | | | | | | | NSLKPEDTALYYCAAATEGHAHALYRLH | |
| | | | | | | | | | YYWGQGTQVTVS | |
| Sb#51 | Nb | SARS-CoV2 | | | | S; RBD | Phage Display | 1058 | QVQLVESGGGLVQAGGSLRLSCAASGF | |
| | | | | | | | Library (Nanobody, | | PVYQGEMHWYRQAPGKEREWVAAIRS | |
| | | | | | | | non-immune) | | TGVQTWYADSVKGRFTISRDNAKNTVY | |
| | | | | | | | | | LQMNSLKPEDTAVYYCRVWVGTHYFG | |
| | | | | | | | | | QGTQVTVS | |
| Sb#52 | Nb | SARS-CoV2 | | | | S; RBD | Phage Display | 1059 | QVQLVESGGGSVQAGGSLRLSCAASGN | |
| | | | | | | | Library (Nanobody, | | IQRIYYLGWFRQAPGKEREGVAALMTYT | |
| | | | | | | | non-immune) | | GHTYYADSVKGRFTVSLDNAKNTVYLQ | |
| | | | | | | | | | MNSLKPEDTALYYCAAAYVGAENPLPYS | |
| | | | | | | | | | MYGYWGQGTQVTVS | |
| Sb#53 | Nb | SARS-CoV2 | | | | S; RBD | Phage Display | 1060 | QVQLVESGGGSVQAGGSLRLSCAASGQ | |
| | | | | | | | Library (Nanobody, | | ISHIKYLGWFRQAPGKEREGVAALITRW | |
| | | | | | | | non-immune) | | GQTYYADSVKGRFTVSLDNAKNTVYLQ | |
| | | | | | | | | | MNSLKPEDTALYYCAAADYGASDPLWFI | |
| | | | | | | | | | HYLYWGQGTQVTVS | |
| Sb#55 | Nb | SARS-CoV2 | | | | S; RBD | Phage Display | 1061 | QVQLVESGGGSVQAGGSLRLSCAASGKI | |
| | | | | | | | Library (Nanobody, | | WTIKYLGWFRQAPGKEREGVAALMTR | |
| | | | | | | | non-immune) | | WGYTYYADSVKGRFTVSLDNAKNTVYL | |
| | | | | | | | | | QMNSLKPEDTALYYCAAANYGSNFPLAE | |
| | | | | | | | | | EDYWYWGQGTQVTVS | |
| Sb#56 | Nb | SARS-CoV2 | | | | S; RBD | Phage Display | 1062 | QVQLVESGGGSVQAGGSLRLSCAASGN | |
| | | | | | | | Library (Nanobody, | | ISQIHYLGWFRQAPGKEREGVAALNTDY | |
| | | | | | | | non-immune) | | GYTYYADSVKGRFTVSLDNAKNTVYLQ | |
| | | | | | | | | | MNSLKPEDTALYYCAAAYYFGDDIPLW | |
| | | | | | | | | | WEAYSYWGQGTQVTVS | |
| Sb#58 | Nb | SARS-CoV2 | | | | S; RBD | Phage Display | 1063 | QVQLVESGGGSVQAGGSLRLSCAASGN | |
| | | | | | | | Library (Nanobody, | | ISTIEYLGWFRQAPGKEREGVAALYTWH | |
| | | | | | | | non-immune) | | GQTYYADSVKGRFTVSLDNAKNTVYLQ | |
| | | | | | | | | | MNSLKPEDTALYYCAAARWGRHMPLS | |
| | | | | | | | | | ATEYSYWGQGTQVTVS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| Sb#41 | N/A | IGHV3-3 | IGHJ4 | N/A | N/A | 3170 | NVKDSGQWR | | N/A | Justin Walter et al., |
| | | (Alpaca) | (Alpaca) | | | | VQYDY | | | 2020 |
| | | | | | | | | | | (https://www.biorxiv.or |
| | | | | | | | | | | g/content/10.1101/202 |
| | | | | | | | | | | 0.04.16.045419v2) |
| Sb#42 | N/A | IGHV3S5 | IGHJ4 | N/A | N/A | 3171 | NVKDHGAQN | | N/A | Justin Walter et al., |
| | | 3 | (Alpaca) | | | | QMYDY | | | 2020 |
| | | (Alpaca) | | | | | | | | (https://www.biorxiv.or |
| | | | | | | | | | | g/content/10.1101/202 |
| | | | | | | | | | | 0.04.16.045419v2) |
| Sb#45 | N/A | IGHV3S5 | IGHJ4 | N/A | N/A | 3172 | NVKDVGHHY | | N/A | Justin Walter et al., |
| | | 3 | (Alpaca) | | | | EYYDY | | | 2020 |
| | | (Alpaca) | | | | | | | | (https://www.biorxiv.or |
| | | | | | | | | | | g/content/10.1101/202 |
| | | | | | | | | | | 0.04.16.045419v2) |
| Sb#46 | N/A | IGHV3S5 | IGHJ4 | N/A | N/A | 3173 | NVKDKGQM | | N/A | Justin Walter et al., |
| | | 3 | (Alpaca) | | | | RAAYDY | | | 2020 |
| | | (Alpaca) | | | | | | | | (https://www.biorxiv.or |
| | | | | | | | | | | g/content/10.1101/202 |
| | | | | | | | | | | 0.04.16.045419v2) |
| Sb#47 | N/A | IGHV3-3 | IGHJ4 | N/A | N/A | 3174 | NVKDYGSSYY | | N/A | Justin Walter et al., |
| | | (Alpaca) | (Alpaca) | | | | KYDY | | | 2020 |
| | | | | | | | | | | (https://www.biorxiv.or |
| | | | | | | | | | | g/content/10.1101/202 |
| | | | | | | | | | | 0.04.16.045419v2) |
| Sb#48 | N/A | IGHV3S5 | IGHJ4 | N/A | N/A | 3175 | NVKDAGSSY | | N/A | Justin Walter et al., |
| | | 3 | (Alpaca) | | | | WDYD | | | 2020 |
| | | (Alpaca) | | | | | | | | (https://www.biorxiv.or |
| | | | | | | | | | | g/content/10.1101/202 |
| | | | | | | | | | | 0.04.16.045419v2) |
| Sb#49 | N/A | IGHV3-3 | IGHJ4 | N/A | N/A | 3176 | AAARWGRTK | | N/A | Justin Walter et al., |
| | | (Alpaca) | (Alpaca) | | | | PLNTYYYSY | | | 2020 |
| | | | | | | | | | | (https://www.biorxiv.or |
| | | | | | | | | | | g/content/10.1101/202 |
| | | | | | | | | | | 0.04.16.045419v2) |
| Sb#5 | N/A | IGHV3-3 | IGHJ4 | N/A | N/A | 3177 | RVHVGAHY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |
| Sb#50 | N/A | IGHV3-3 | IGHJ4 | N/A | N/A | 3178 | AAATEGHAH | | N/A | Justin Walter et al., |
| | | (Alpaca) | (Alpaca) | | | | ALYRLHYY | | | 2020 |
| | | | | | | | | | | (https://www.biorxiv.or |
| | | | | | | | | | | g/content/10.1101/202 |
| | | | | | | | | | | 0.04.16.045419v2) |
| Sb#51 | N/A | IGHV3-3 | IGHJ4 | N/A | N/A | 3179 | RVWVGTHY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |
| Sb#52 | N/A | IGHV3-3 | IGHJ4 | N/A | N/A | 3180 | AAAYVGAEN | | N/A | Justin Walter et al., |
| | | (Alpaca) | (Alpaca) | | | | PLPYSMYGY | | | 2020 |
| | | | | | | | | | | (https://www.biorxiv.or |
| | | | | | | | | | | g/content/10.1101/202 |
| | | | | | | | | | | 0.04.16.045419v2) |
| Sb#53 | N/A | IGHV3-3 | IGHJ4 | N/A | N/A | 3181 | AAADYGASD | | N/A | Justin Walter et al., |
| | | (Alpaca) | (Alpaca) | | | | PLWFIHYLY | | | 2020 |
| | | | | | | | | | | (https://www.biorxiv.or |
| | | | | | | | | | | g/content/10.1101/202 |
| | | | | | | | | | | 0.04.16.045419v2) |
| Sb#55 | N/A | IGHV3-3 | IGHJ4 | N/A | N/A | 3182 | AAANYGSNFP | | N/A | Justin Walter et al., |
| | | (Alpaca) | (Alpaca) | | | | LAEEDYWY | | | 2020 |
| | | | | | | | | | | (https://www.biorxiv.or |
| | | | | | | | | | | g/content/10.1101/202 |
| | | | | | | | | | | 0.04.16.045419v2) |
| Sb#56 | N/A | IGHV3-3 | IGHJ4 | N/A | N/A | 3183 | AAAYYFGDDI | | N/A | Justin Walter et al., |
| | | (Alpaca) | (Alpaca) | | | | PLWWEAYSY | | | 2020 |
| | | | | | | | | | | (https://www.biorxiv.or |
| | | | | | | | | | | g/content/10.1101/202 |
| | | | | | | | | | | 0.04.16.045419v2) |
| Sb#58 | N/A | IGHV3-3 | IGHJ4 | N/A | N/A | 3184 | AAARWGRH | | N/A | Justin Walter et al., |
| | | (Alpaca) | (Alpaca) | | | | MPLSATEYSY | | | 2020 |
| | | | | | | | | | | (https://www.biorxiv.or |
| | | | | | | | | | | g/content/10.1101/202 |
| | | | | | | | | | | 0.04.16.045419v2) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| Sb#59 | Nb | SARS-CoV2 | | | | S; RBD | Phage Display | 1064 | QVQLVESGGGSVQAGGSLRLSCAASGN | |
| | | | | | | | Library (Nanobody, | | IESIYYLGWFRQAPGKEREGVAALWTG | |
| | | | | | | | non-immune) | | DGETYYADSVKGRFTVSLDNAKNTVYLQ | |
| | | | | | | | | | MNSLKPEDTALYYCAAAAWGNSAPLTT | |
| | | | | | | | | | YRYYYWGQGTQVTVS | |
| Sb#6 | Nb | SARS-CoV2 | | | | S; RBD | Phage Display | 1065 | QVQLVESGGGLVQAGGSLRLSCAASGF | |
| | | | | | | | Library (Nanobody, | | PVEQGRMYWYRQAPGKEREWVAAIIS | |
| | | | | | | | non-immune) | | HGTVTVYADSVKGRFTISRDNAKNTVYL | |
| | | | | | | | | | QMNSLKPEDTAVYYCYVYVGAQYWGQ | |
| | | | | | | | | | GTQVTVS | |
| Sb#61 | Nb | SARS-CoV2 | | | | S; RBD | Phage Display | 1066 | QVQLVESGGGSVQAGGSLRLSCAASGFI | |
| | | | | | | | Library (Nanobody, | | YGITYLGWFRQAPGKEREGVAALVTWN | |
| | | | | | | | non-immune) | | GQTYYADSVKGRFTVSLDNAKNTVYLQ | |
| | | | | | | | | | MNSLKPEDTALYYCAAADWGYDWPLW | |
| | | | | | | | | | DEWYWYWGQGTQVTVS | |
| Sb#62 | Nb | SARS-CoV2 | | | | S; RBD | Phage Display | 1067 | QVQLVESGGGSVQAGGSLRLSCAASGTI | |
| | | | | | | | Library (Nanobody, | | ADIKYLGWFRQAPGKEREGVAALMTR | |
| | | | | | | | non-immune) | | WGSTYYADSVKGRFTVSLDNAKNTVYL | |
| | | | | | | | | | QMNSLKPEDTALYYCAAANYGANYPLYS | |
| | | | | | | | | | QQYSYWGQGTQVTVS | |
| Sb#63 | Nb | SARS-CoV2 | | | | S; RBD | Phage Display | 1068 | QVQLVESGGGSVQAGGSLRLSCAASGSI | |
| | | | | | | | Library (Nanobody, | | SSIKYLGWFRQAPGKEREGVAALMTRW | |
| | | | | | | | non-immune) | | GMTYYADSVKGRFTVSLDNAKNTVYLQ | |
| | | | | | | | | | MNSLKPEDTALYYCAAANYGANEPLQY | |
| | | | | | | | | | THYNYWGQGTQVTVS | |
| Sb#64 | Nb | SARS-CoV2 | | | | S; RBD | Phage Display | 1069 | QVQLVESGGGSVQAGGSLRLSCAASGEI | |
| | | | | | | | Library (Nanobody, | | ESIFYLGWFRQAPGKEREGVAALYTYVG | |
| | | | | | | | non-immune) | | QTYYADSVKGRFTVSLDNAKNTVYLQM | |
| | | | | | | | | | NSLKPEDTALYYCAAASYGAAHPLSIMR | |
| | | | | | | | | | YYYWGQGTQVTVS | |
| Sb#65 | Nb | SARS-CoV2 | | | | S; RBD | Phage Display | 1070 | QVQLVESGGGSVQAGGSLRLSCAASGTI | |
| | | | | | | | Library (Nanobody, | | AHIKYLGWFRQAPGKEREGVAALMTK | |
| | | | | | | | non-immune) | | WGQTYYADSVKGRFTVSLDNAKNTVYL | |
| | | | | | | | | | QMNSLKPEDTALYYCAAASYGANFPLKA | |
| | | | | | | | | | SDYSYWGQGTQVTVS | |
| Sb#66 | Nb | SARS-CoV2 | | | | S; RBD | Phage Display | 1071 | QVQLVESGGGSVQAGGSLRLSCAASGSI | |
| | | | | | | | Library (Nanobody, | | QAITYLGWFRQAPGKEREGVAALVTW | |
| | | | | | | | non-immune) | | NGQTYYADSVKGRFTVSLDNAKNTVYL | |
| | | | | | | | | | QMNSLKPEDTALYYCAAADWGYDWPL | |
| | | | | | | | | | WDEWYWYWGQGTQVTVS | |
| Sb#67 | Nb | SARS-CoV2 | | | | S; RBD | Phage Display | 1072 | QVQLVESGGGSVQAGGSLRLSCAASGSI | |
| | | | | | | | Library (Nanobody, | | SSITYLGWFRQAPGKEREGVAALVTYSG | |
| | | | | | | | non-immune) | | NTYYADSVKGRFTVSLDNAKNTVYLQM | |
| | | | | | | | | | NSLKPEDTALYYCAAATWGHSWPLYND | |
| | | | | | | | | | EYWYWGQGSQVTVS | |
| Sb#68 | Nb | SARS-CoV2 | | | | S; RBD | Phage Display | 1073 | QVQLVESGGGSVQAGGSLRLSCAASGSI | |
| | | | | | | | Library (Nanobody, | | SSITYLGWFRQAPGKEREGVAALITVNG | |
| | | | | | | | non-immune) | | HTYYADSVKGRFTVSLDNAKNTVYLQM | |
| | | | | | | | | | NSLKPEDTALYYCAAAAWGYAWPLHQ | |
| | | | | | | | | | DDYWYWGQGTQVTVS | |
| Sb#69 | Nb | SARS-CoV2 | | | | S; RBD | Phage Display | 1074 | QVQLVESGGGSVQAGGSLRLSCAASGSI | |
| | | | | | | | Library (Nanobody, | | SSITYLGWFRQAPGKEREGVAALNTFNG | |
| | | | | | | | non-immune) | | TTYYADSVKGRFTVSLDNAKNTVYLQM | |
| | | | | | | | | | NSLKPEDTALYYCAAATWGYSWPLIAEY | |
| | | | | | | | | | NWYWGQGTQVTVS | |
| Sb#7 | Nb | SARS-CoV2 | | | | S; RBD | Phage Display | 1075 | QVQLVESGGGLVQAGGSLRLSCAASGF | |
| | | | | | | | Library (Nanobody, | | PVLFTYMHWYRQAPGKEREWVAAIWS | |
| | | | | | | | non-immune) | | SGNSTWYADSVKGRFTISRDNAKNTVYL | |
| | | | | | | | | | QMNSLKPEDTAVYYCFVKVGNWYAGQ | |
| | | | | | | | | | GTQVTVS | |
| Sb#71 | Nb | SARS-CoV2 | | | | S; RBD | Phage Display | 1076 | QVQLVESGGGSVQAGGSLRLSCAASGSI | |
| | | | | | | | Library (Nanobody, | | SSITYLGWFRQAPGKEREGVAALKTQAG | |
| | | | | | | | non-immune) | | FTYYADSVKGRFTVSLDNAKNTVYLQM | |
| | | | | | | | | | NSLKPEDTALYYCAAANWGYSWPLYEA | |
| | | | | | | | | | DDWYWGQGTQVTVS | |
| Sb#8 | Nb | SARS-CoV2 | | | | S; RBD | Phage Display | 1077 | QVQLVESGGGLVQAGGSLRLSCAASGF | |
| | | | | | | | Library (Nanobody, | | PVNAGNMHWYRQAPGKEREWVAAIQ | |
| | | | | | | | non-immune) | | SYGRTTYYADSVKGRFTISRDNAKNTVYL | |
| | | | | | | | | | QMNSLKPEDTAVYYCRVFVGMHYFGQ | |
| | | | | | | | | | GTQVTVS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| Sb#59 | N/A | IGHV3-3 | IGHJ4 | N/A | N/A | 3185 | AAAAWGNSA | | N/A | Justin Walter et al., |
| | | (Alpaca) | (Alpaca) | | | | PLTTYRYYY | | | 2020 |
| | | | | | | | | | | (https://www.biorxiv.or |
| | | | | | | | | | | g/content/10.1101/202 |
| | | | | | | | | | | 0.04.16.045419v2) |
| Sb#6 | N/A | IGHV3-3 | IGHJ4 | N/A | N/A | 3186 | YVYVGAQY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |
| Sb#61 | N/A | IGHV3-3 | IGHJ4 | N/A | N/A | 3187 | AAADWGYD | | N/A | Justin Walter et al., |
| | | (Alpaca) | (Alpaca) | | | | WPLWDEWY | | | 2020 |
| | | | | | | | WY | | | (https://www.biorxiv.or |
| | | | | | | | | | | g/content/10.1101/202 |
| | | | | | | | | | | 0.04.16.045419v2) |
| Sb#62 | N/A | IGHV3-3 | IGHJ4 | N/A | N/A | 3188 | AAANYGANY | | N/A | Justin Walter et al., |
| | | (Alpaca) | (Alpaca) | | | | PLYSQQYSY | | | 2020 |
| | | | | | | | | | | (https://www.biorxiv.or |
| | | | | | | | | | | g/content/10.1101/202 |
| | | | | | | | | | | 0.04.16.045419v2) |
| Sb#63 | N/A | IGHV3-3 | IGHJ4 | N/A | N/A | 3189 | AAANYGANE | | N/A | Justin Walter et al., |
| | | (Alpaca) | (Alpaca) | | | | PLQYTHYNY | | | 2020 |
| | | | | | | | | | | (https://www.biorxiv.or |
| | | | | | | | | | | g/content/10.1101/202 |
| | | | | | | | | | | 0.04.16.045419v2) |
| Sb#64 | N/A | IGHV3-3 | IGHJ4 | N/A | N/A | 3190 | AAASYGAAHP | | N/A | Justin Walter et al., |
| | | (Alpaca) | (Alpaca) | | | | LSIMRYYY | | | 2020 |
| | | | | | | | | | | (https://www.biorxiv.or |
| | | | | | | | | | | g/content/10.1101/202 |
| | | | | | | | | | | 0.04.16.045419v2) |
| Sb#65 | N/A | IGHV3-3 | IGHJ4 | N/A | N/A | 3191 | AAASYGANFP | | N/A | Justin Walter et al., |
| | | (Alpaca) | (Alpaca) | | | | LKASDYSY | | | 2020 |
| | | | | | | | | | | (https://www.biorxiv.or |
| | | | | | | | | | | g/content/10.1101/202 |
| | | | | | | | | | | 0.04.16.045419v2) |
| Sb#66 | N/A | IGHV3-3 | IGHJ4 | N/A | N/A | 3192 | AAADWGYD | | N/A | Justin Walter et al., |
| | | (Alpaca) | (Alpaca) | | | | WPLWDEWY | | | 2020 |
| | | | | | | | WY | | | (https://www.biorxiv.or |
| | | | | | | | | | | g/content/10.1101/202 |
| | | | | | | | | | | 0.04.16.045419v2) |
| Sb#67 | N/A | IGHV3-3 | IGHJ4 | N/A | N/A | 3193 | AAATWGHS | | N/A | Justin Walter et al., |
| | | (Alpaca) | (Alpaca) | | | | WPLYNDEYW | | | 2020 |
| | | | | | | | Y | | | (https://www.biorxiv.or |
| | | | | | | | | | | g/content/10.1101/202 |
| | | | | | | | | | | 0.04.16.045419v2) |
| Sb#68 | N/A | IGHV3-3 | IGHJ4 | N/A | N/A | 3194 | AAAAWGYA | | N/A | Justin Walter et al., |
| | | (Alpaca) | (Alpaca) | | | | WPLHQDDY | | | 2020 |
| | | | | | | | WY | | | (https://www.biorxiv.or |
| | | | | | | | | | | g/content/10.1101/202 |
| | | | | | | | | | | 0.04.16.045419v2) |
| Sb#69 | N/A | IGHV3-3 | IGHJ5 | N/A | N/A | 3195 | AAATWGYSW | | N/A | Justin Walter et al., |
| | | (Alpaca) | (Alpaca) | | | | PLIAEYNWY | | | 2020 |
| | | | | | | | | | | (https://www.biorxiv.or |
| | | | | | | | | | | g/content/10.1101/202 |
| | | | | | | | | | | 0.04.16.045419v2) |
| Sb#7 | N/A | IGHV3-3 | IGHJ4 | N/A | N/A | 3196 | FVKVGNWY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |
| Sb#71 | N/A | IGHV3-3 | IGHJ4 | N/A | N/A | 3197 | AAANWGYS | | N/A | Justin Walter et al., |
| | | (Alpaca) | (Alpaca) | | | | WPLYEADDW | | | 2020 |
| | | | | | | | Y | | | (https://www.biorxiv.or |
| | | | | | | | | | | g/content/10.1101/202 |
| | | | | | | | | | | 0.04.16.045419v2) |
| Sb#8 | N/A | IGHV3-3 | IGHJ4 | N/A | N/A | 3198 | YVYVGGSY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| Sb#9 | Nb | SARS-CoV2 | | | | S; RBD | Phage Display | 1078 | QVQLVESGGGLVQAGGSLRLSCAASGF | |
| | | | | | | | Library (Nanobody, | | PVSSSTMTWYRQAPGKEREWVAAINSY | |
| | | | | | | | non-immune) | | GWETHYADSVKGRFTISRDNAKNTVYL | |
| | | | | | | | | | QMNSLKPEDTAVYYCYVYVGGSYIGQG | |
| | | | | | | | | | TQVTVS | |
| Sb100 | Nb | SARS-CoV2 | | SARS-CoV2 | | S; RBD | Phage Display | 1079 | QVQLVESGGGSVQAGGSLRLSCAASGSI | |
| | | | | | | | Library (Nanobody, | | SSITYLGWFRQAPGKEREGVAALVTSDG | |
| | | | | | | | non-immune) | | RTYYADSVKGRFTVSLDNAKNTVYLQM | |
| | | | | | | | | | NSLKPEDTALYYCAAANWGYSWPLYQT | |
| | | | | | | | | | EYWYWGQGTQVTVSS | |
| Sb12 | Nb | SARS-CoV2 | | SARS-CoV2 | | S; RBD | Phage Display | 1080 | QVQLVESGGGLVQAGGSLRLSCAASGF | |
| | | | | | | | Library (Nanobody, | | PVQLYWMEWYRQAPGKEREWVAAITS | |
| | | | | | | | non-immune) | | DGDYTEYADSVKGRFTISRDNAKNTVYL | |
| | | | | | | | | | QMNSLKPEDTAVYYCYVKVGEWYYGQ | |
| | | | | | | | | | GTQVTVSS | |
| Sb13 | Nb | SARS-CoV2 | | | | S; RBD | Phage Display | 1081 | QVQLVESGGGLVQAGGSLRLSCAASGF | |
| | | (weak) | | | | | Library (Nanobody, | | PVENYYMRWYRQAPGKEREWVAAIES | |
| | | | | | | | non-immune) | | SGAETRYADSVKGRFTISRDNAKNTVYL | |
| | | | | | | | | | QMNSLKPEDTAVYYCYVVVGWGYAGQ | |
| | | | | | | | | | GTQVTVSS | |
| Sb15 | Nb | SARS-CoV2 | | | | S; RBD | Phage Display | 1082 | QVQLVESGGGLVQAGGSLRLSCAASGF | |
| | | | | | | | Library (Nanobody, | | PVYEHYMRWYRQAPGKEREWVAAIQS | |
| | | | | | | | non-immune) | | HGNHTAYADSVKGRFTISRDNAKNTVYL | |
| | | | | | | | | | QMNSLKPEDTAVYYCFVVVGNGYTGQ | |
| | | | | | | | | | GTQVTVSS | |
| Sb16 | Nb | SARS-CoV2 | | | | S; RBD | Phage Display | 1083 | QVQLVESGGGLVRAGGSLRLSCAASGF | |
| | | | | | | | Library (Nanobody, | | PVASQEMTWYRQAPGKEREWVAAISSS | |
| | | | | | | | non-immune) | | GRQTEYADSVKGRFTISRDNAKNTVYLQ | |
| | | | | | | | | | MNSLKPEDTAVYYCYVYVGGSYIGQGT | |
| | | | | | | | | | QVTVSS | |
| Sb17 | Nb | SARS-CoV2 | | SARS-CoV2 | | S; RBD | Phage Display | 1084 | QVQLVESGGGLVQAGGSLRLSCAASGF | |
| | | | | | | | Library (Nanobody, | | PVKASEMEWYRQAPGKEREWVAAIASI | |
| | | | | | | | non-immune) | | GYNTYYADSVKGRFTISRDNAKNTVYLQ | |
| | | | | | | | | | MNSLKPEDTAVYYCLVYVGATYIGQGTQ | |
| | | | | | | | | | VTVSS | |
| Sb2 | Nb | SARS-CoV2 | | | | S; RBD | Phage Display | 1085 | QVQLVESGGGLVQAGGSLRLSCAASGF | |
| | | | | | | | Library (Nanobody, | | PVSNEEMTWYRQAPGKEREWVAAIAS | |
| | | | | | | | non-immune) | | NGNQTEYADSVKGRFTISRDNAKNTVYL | |
| | | | | | | | | | QMNSLKPEDTAVYYCYVYVGASYIGQG | |
| | | | | | | | | | TQVTVSS | |
| Sb21 | Nb | SARS-CoV2 | | | | S; RBD | Phage Display | 1086 | QVQLVESGGGLVQAGGSLRLSCAASGF | |
| | | | | | | | Library (Nanobody, | | PVKESEMTWYRQARGKEREWVAAINS | |
| | | | | | | | non-immune) | | HGMTTHYADSVKGRFTISRDNAKNTVY | |
| | | | | | | | | | LQMNSLKPEDTAVYYCYVYVGGSYIGQ | |
| | | | | | | | | | GTQVTVSS | |
| Sb22 | Nb | SARS-CoV2 | | SARS-CoV2 | | S; RBD | Phage Display | 1087 | QVQLVESGGGLVQAGGSLRLSCAASGF | |
| | | | | | | | Library (Nanobody, | | PVNHYEMEWYRQAPGREREWVAAIM | |
| | | | | | | | non-immune) | | DSTGYETAYADSVKGRFTISRDNAKNTV | |
| | | | | | | | | | YLQMNSLKPEDTAVYYCYVYVGASYIGQ | |
| | | | | | | | | | GTQVTVSS | |
| Sb23 | Nb | SARS-CoV2 | | SARS-CoV2 | | S; RBD | Phage Display | 1088 | QVQLVESGGGLVQAGGSLRLSCAASGF | |
| | | | | | | | Library (Nanobody, | | PVESENMHWYRQAPGKEREWVAAIYS | |
| | | | | | | | non-immune) | | TGGWTLYADSVKGRFTISRDNAKNTVYL | |
| | | | | | | | | | QMNSLKPEDTAVYYCAVQVGYWYEGQ | |
| | | | | | | | | | GTQVTVSS | |
| Sb25 | Nb | SARS-CoV2 | | | | S; RBD | Phage Display | 1089 | QVQLVESGGGLVQAGGSLRLSCAASGF | |
| | | | | | | | Library (Nanobody, | | PVESTEMTWYRQAPGKEREWVAAIESE | |
| | | | | | | | non-immune) | | GHGTEYADSVKGRFTISRDNAKNTVYLQ | |
| | | | | | | | | | MNSLKPEDTAVYYCYVYVGAGYIGQGT | |
| | | | | | | | | | QVTVSS | |
| Sb27 | Nb | SARS-CoV2 | | | | S; RBD | Phage Display | 1090 | QVQLVESGGGLVQAGGSLRLSCAASGF | |
| | | | | | | | Library (Nanobody, | | PVKASEMVWYRQAPGKEREWVAAILS | |
| | | | | | | | non-immune) | | QGHATEYADSVKGRFTISRDNAKNTVYL | |
| | | | | | | | | | QMNSLKPEDTAVYYCYVWVGRSYIGQG | |
| | | | | | | | | | TQVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| Sb#9 | N/A | IGHV3-3 | IGHJ4 | N/A | N/A | 3199 | RVFVGMHY | | N/A | Justin Walter et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.04.16.045419v2) |
| Sb100 | N/A | IGHV3-3 | IGHJ4 | N/A | N/A | 3200 | AAANWGYS | | N/A | Tania Custodia et al., |
| | | (Alpaca) | (Alpaca) | | | | WPLYQTEYW | | | 2020 |
| | | | | | | | Y | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb12 | N/A | IGHV3-3 | IGHJ4 | N/A | N/A | 3201 | YVKVGEWY | | N/A | Tania Custodia et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb13 | N/A | IGHV3-3 | IGHJ4 | N/A | N/A | 3202 | YVVVGWGY | | N/A | Tania Custodia et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb15 | N/A | IGHV3-3 | IGHJ4 | N/A | N/A | 3203 | FVVVGNGY | | N/A | Tania Custodia et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb16 | N/A | IGHV3-3 | IGHJ4 | N/A | N/A | 3204 | YVYVGGSY | | N/A | Tania Custodia et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb17 | N/A | IGHV3-3 | IGHJ4 | N/A | N/A | 3205 | LVYVGATY | | N/A | Tania Custodia et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb2 | N/A | IGHV3-3 | IGHJ4 | N/A | N/A | 3206 | YVYVGASY | | N/A | Tania Custodia et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb21 | N/A | IGHV3-3 | IGHJ4 | N/A | N/A | 3207 | YVYVGGSY | | N/A | Tania Custodia et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb22 | N/A | IGHV3-3 | IGHJ4 | N/A | N/A | 3208 | YVYVGASY | | N/A | Tania Custodia et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb23 | N/A | IGHV3-3 | IGHJ4 | N/A | N/A | 3209 | AVQVGYWY | | N/A | Tania Custodia et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb25 | N/A | IGHV3-3 | IGHJ4 | N/A | N/A | 3210 | YVYVGAGY | | N/A | Tania Custodia et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb27 | N/A | IGHV3-3 | IGHJ4 | N/A | N/A | 3211 | YVWVGRSY | | N/A | Tania Custodia et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| Sb28 | Nb | SARS-CoV2 | | | | S; RBD | Phage Display | 1091 | QVQLVESGGGLVQAGGSLRLSCAASGF | |
| | | | | | | | Library (Nanobody, | | PVYSAEMEWYRQAPGKEREWVAAISSY | |
| | | | | | | | non-immune) | | GTNTYYADSVKGRFTISRDNAKNTVYLQ | |
| | | | | | | | | | MNSLKPEDTAVYYCYVYVGSSYIGQGTQ | |
| | | | | | | | | | VTVSS | |
| Sb30 | Nb | SARS-CoV2 | | | | S; RBD | Phage Display | 1092 | QVQLVESGGGLVQAGGSLRLSCAASGF | |
| | | | | | | | Library (Nanobody, | | PVWYKEMEWYRQAPGKEREWVAAITS | |
| | | | | | | | non-immune) | | AGHHTYYADSVKGRFTISRDNAKNTVYL | |
| | | | | | | | | | QMNSLKPEDTAVYYCYVYVGESYIGQGT | |
| | | | | | | | | | QVTVSS | |
| Sb32 | Nb | SARS-CoV2 | | | | S; RBD | Phage Display | 1093 | QVQLVESGGGLVQAGGSLRLSCAASGF | |
| | | | | | | | Library (Nanobody, | | PVAHKSMWWYRQAPGKEREWVAAIES | |
| | | | | | | | non-immune) | | TGDTTRYADSVKGRFTISRDNAKNTVYL | |
| | | | | | | | | | QMNSLKPEDTAVYYCVVWVGEVYRGQ | |
| | | | | | | | | | GTQVTVSS | |
| Sb37 | Nb | SARS-CoV2 | | | | S; RBD | Phage Display | 1094 | QVQLVESGGGLVQAGGSLRLSCAASGF | |
| | | (weak) | | | | | Library (Nanobody, | | PVYNTWMEWYRQAPGKEREWVAAITS | |
| | | | | | | | non-immune) | | YGFHTYYADSVKGRFTISRDNAKNTVYL | |
| | | | | | | | | | QMNSLKPEDTAVYYCNVKDEGNTTAYY | |
| | | | | | | | | | DYWGQGTQVTVSS | |
| Sb38 | Nb | SARS-CoV2 | | | | S; RBD | Phage Display | 1095 | QVQLVESGGGLVQAGGSLRLSCAASGF | |
| | | | | | | | Library (Nanobody, | | PVYWAHMTWYRQAPGKEREWVAAIV | |
| | | | | | | | non-immune) | | SSGAYTAYADSVKGRFTISRDNAKNTVYL | |
| | | | | | | | | | QMNSLKPEDTAVYYCNVKDFGTQEHYY | |
| | | | | | | | | | DYWGQGTQVTVSS | |
| Sb39 | Nb | SARS-CoV2 | | | | S; RBD | Phage Display | 1096 | QVQLVESGGGPVQAGGSLRLSCAASGF | |
| | | | | | | | Library (Nanobody, | | PVMWSHMHWYRQAPGKEREWVAAIV | |
| | | | | | | | non-immune) | | SYGAYTIYADSVKGRFTISRDNAKNTVYL | |
| | | | | | | | | | QMNSLKPEDTAVYYCNVKDFGGYRYYY | |
| | | | | | | | | | DYWGQGTQVTVSS | |
| Sb40 | Nb | SARS-CoV2 | | | | S; RBD | Phage Display | 1097 | QVQLVESGGGLVQAGGSLRLSCAASGF | |
| | | | | | | | Library (Nanobody, | | PVQGTWMEWYRQAPGKEREWVAAIT | |
| | | | | | | | non-immune) | | SVGYRTYYADSVKGRFTISRDNAKNTVYL | |
| | | | | | | | | | QMNSLKPEDTAVYYCNVKDEGAIAKNY | |
| | | | | | | | | | DYWGQGTQVTVSS | |
| Sb42 | Nb | SARS-CoV2 | | SARS-CoV2 | | S; RBD | Phage Display | 1098 | QVQLVESGGGLVQAGGSLRLSCAASGF | |
| | | | | | | | Library (Nanobody, | | PVYNTWMEWYRQAPGKEREWVAAITS | |
| | | | | | | | non-immune) | | WGFKTYYADSVKGRFTISRDNAKNTVYL | |
| | | | | | | | | | QMNSLKPEDTAVYYCNVKDEGYTGYYY | |
| | | | | | | | | | DYWGQGTQVTVSS | |
| Sb43 | Nb | SARS-CoV2 | | SARS-CoV2 | | S; RBD | Phage Display | 1099 | QVQLVESGGGLVQAGGSLRLSCAASGF | |
| | | | | | | | Library (Nanobody, | | PVEWTHMHWYRQAPGKEREWVAAIA | |
| | | | | | | | non-immune) | | SSGAYTVYADSVKGRFTISRDNAKNTVYL | |
| | | | | | | | | | QMNSLKPEDTAVYYCNVKDWGSQDRY | |
| | | | | | | | | | YDYWGQGTQVTVSS | |
| Sb45 | Nb | SARS-CoV2 | | | | S; RBD | Phage Display | 1100 | QVQLVESGGGLVQAGGSLRLSCAASGF | |
| | | | | | | | Library (Nanobody, | | PVAGTWMEWYRQAPGKEREWVAAITS | |
| | | | | | | | non-immune) | | YGYRTYYADSVKGRFTISRDNAKNTVYL | |
| | | | | | | | | | QMNSLKPEDTAVYYCNVKDEGKSSQVY | |
| | | | | | | | | | DYWGQGTQVTVSS | |
| Sb46 | Nb | SARS-CoV2 | | SARS-CoV2 | | S; RBD | Phage Display | 1101 | QVQLVESGGGLVQAGGSLRLSCAASGF | |
| | | | | | | | Library (Nanobody, | | PVNYTIMFWYRKAPGKEREWVAAIKSH | |
| | | | | | | | non-immune) | | GATTLYADSVKGRFTISRDNAKNTVYLQ | |
| | | | | | | | | | MNSLKPEDTAVYYCNVKDVGNDQKSYD | |
| | | | | | | | | | YWGQGTQVTVSS | |
| Sb47 | Nb | SARS-CoV2 | | | | S; RBD | Phage Display | 1102 | QVQLVESGGGLVQAGGSLRLSCAASGF | |
| | | | | | | | Library (Nanobody, | | PVVWAHMHWYRQAPGKEREWVAAIT | |
| | | | | | | | non-immune) | | SEGAHTIYADSVKGRFTISRDNAKNTVYL | |
| | | | | | | | | | QMNSLKPEDTAVYYCNVKDWGTYSTYY | |
| | | | | | | | | | DYWGQGTQVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| Sb28 | N/A | IGHV3-3 | IGHJ4 | N/A | N/A | 3212 | YVYVGSSY | | N/A | Tania Custodia et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb30 | N/A | IGHV3-3 | IGHJ4 | N/A | N/A | 3213 | YVYVGESY | | N/A | Tania Custodia et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb32 | N/A | IGHV3-3 | IGHJ4 | N/A | N/A | 3214 | VVWVGEVY | | N/A | Tania Custodia et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb37 | N/A | IGHV3S5 | IGHJ4 | N/A | N/A | 3215 | NVKDEGNTT | | N/A | Tania Custodia et al., |
| | | 3 | (Alpaca) | | | | AYYDY | | | 2020 |
| | | (Alpaca) | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb38 | N/A | IGHV3S5 | IGHJ4 | N/A | N/A | 3216 | NVKDFGTQE | | N/A | Tania Custodia et al., |
| | | 3 | (Alpaca) | | | | HYYDY | | | 2020 |
| | | (Alpaca) | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb39 | N/A | IGHV3S5 | IGHJ4 | N/A | N/A | 3217 | NVKDFGGYR | | N/A | Tania Custodia et al., |
| | | 3 | (Alpaca) | | | | YYYDY | | | 2020 |
| | | (Alpaca) | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb40 | N/A | IGHV3S5 | IGHJ4 | N/A | N/A | 3218 | NVKDEGAIAK | | N/A | Tania Custodia et al., |
| | | 3 | (Alpaca) | | | | NYDY | | | 2020 |
| | | (Alpaca) | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb42 | N/A | IGHV3S5 | IGHJ4 | N/A | N/A | 3219 | NVKDEGYTGY | | N/A | Tania Custodia et al., |
| | | 3 | (Alpaca) | | | | YYDY | | | 2020 |
| | | (Alpaca) | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb43 | N/A | IGHV3-3 | IGHJ4 | N/A | N/A | 3220 | NVKDWGSQ | | N/A | Tania Custodia et al., |
| | | (Alpaca) | (Alpaca) | | | | DRYYDY | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb45 | N/A | IGHV3S5 | IGHJ4 | N/A | N/A | 3221 | NVKDEGKSS | | N/A | Tania Custodia et al., |
| | | 3 | (Alpaca) | | | | QVYDY | | | 2020 |
| | | (Alpaca) | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb46 | N/A | IGHV3S5 | IGHJ4 | N/A | N/A | 3222 | NVKDVGNDQ | | N/A | Tania Custodia et al., |
| | | 3 | (Alpaca) | | | | KSYDY | | | 2020 |
| | | (Alpaca) | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb47 | N/A | IGHV3S5 | IGHJ4 | N/A | N/A | 3223 | NVKDWGTYS | | N/A | Tania Custodia et al., |
| | | 3 | (Alpaca) | | | | TYYDY | | | 2020 |
| | | (Alpaca) | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| Sb5 | Nb | SARS-CoV2 | | | | S; RBD | Phage Display | 1103 | QVQLVESGGGLVQAGGSLRLSCAASGF | |
| | | (weak) | | | | | Library (Nanobody, | | PVAQQEMTWYRQAPGKEREWVAAISS | |
| | | | | | | | non-immune) | | IGSITHYADSVKGRFTISRDNAKNTVYLQ | |
| | | | | | | | | | MNSLKPEDTAVYYCYVYVGASYIGQGT | |
| | | | | | | | | | QVTVSS | |
| Sb50 | Nb | SARS-CoV2 | | | | S; RBD | Phage Display | 1104 | QVQLVESGGGLVQAGGSLRLSCAASGF | |
| | | | | | | | Library (Nanobody, | | PVQYEHMHWYRQAPGKEREWVAAIVS | |
| | | | | | | | non-immune) | | EGAYTHYADSVKGRFTISRDNAKNTVYL | |
| | | | | | | | | | QMNSLKPEDTAVYYCNVKDWGWLAQY | |
| | | | | | | | | | YDYWGQGTQVTVSS | |
| Sb52 | Nb | SARS-CoV2 | | | | S; RBD | Phage Display | 1105 | QVQLVESGGGLVQAGGSLRLSCAASGF | |
| | | | | | | | Library (Nanobody, | | PVNYNWMEWYRQAPGKEREWVAAIT | |
| | | | | | | | non-immune) | | SWGYKTYYADSVKGRFTISRDNAKNTVY | |
| | | | | | | | | | LQMNSLKPEDTAVYYCNVKDEGMWQ | |
| | | | | | | | | | HYYDYWGQGTQVTVSS | |
| Sb54 | Nb | SARS-CoV2 | | | | S; RBD | Phage Display | 1106 | QVQLVESGGGLVQAGGSLRLSCAASGF | |
| | | | | | | | Library (Nanobody, | | PVWNTWMEWYRQAPGKEREWVAAIT | |
| | | | | | | | non-immune) | | SYGFKTYYADSVKGRFTISRDNAKNTVYL | |
| | | | | | | | | | QMNSLKPEDTAVYYCNVKDEGNSQSHY | |
| | | | | | | | | | DYWGQGTQVTVSS | |
| Sb56 | Nb | SARS-CoV2 | | | | S; RBD | Phage Display | 1107 | QVQLVESGGGLVQAGGSLRLSCAASGF | |
| | | | | | | | Library (Nanobody, | | PVEMWSMEWYRQAPGKEREWVAAI | |
| | | | | | | | non-immune) | | MSFGYQTWYADSVKGRFTISRDNAKNT | |
| | | | | | | | | | VYLQMNSLKPEDTAVYYCNVKDAGNSK | |
| | | | | | | | | | ALYDYWGQGTQVTVSS | |
| Sb57 | Nb | SARS-CoV2 | | | | S; RBD | Phage Display | 1108 | QVQLVESGGGLVQAGGSLRLSCAASGF | |
| | | | | | | | Library (Nanobody, | | PVEHDHMHWYRQAPGKEREWVAAIVS | |
| | | | | | | | non-immune) | | QGAYTVYADSVKGRFTISRDNAKNTVYL | |
| | | | | | | | | | QMNSLKPEDTAVYYCNVKDWGRAGAR | |
| | | | | | | | | | YDYWGQGTQVTVSS | |
| Sb58 | Nb | SARS-CoV2 | | | | S; RBD | Phage Display | 1109 | QVQLVESGGGLVQAGGSLRLSCAASGF | |
| | | (weak) | | | | | Library (Nanobody, | | PVDAAWMEWYRQAPGKEREWVAAIT | |
| | | | | | | | non-immune) | | SYGYRTYYADSVKGRFTISRDNAKNTVYL | |
| | | | | | | | | | QMNSLKPEDTAVYYCNVKDMDRWRTT | |
| | | | | | | | | | YDYWGQGTQVTVSS | |
| Sb6 | Nb | SARS-CoV2 | | | | S; RBD | Phage Display | 1110 | QVQLVESGGGLVQAGGSLRLSCAASGF | |
| | | | | | | | Library (Nanobody, | | PVNHTRMYWYRQAPGKEREWVAAIQS | |
| | | | | | | | non-immune) | | HGQNTFYADSVKGRFTISRDNAKNTVYL | |
| | | | | | | | | | QMNSLKPEDTAVYYCYVWVGNQYWG | |
| | | | | | | | | | QGTQVTVSS | |
| Sb60 | Nb | SARS-CoV2 | | | | S; RBD | Phage Display | 1111 | QVQLVESGGGLVQAGGSLRLSCAASGF | |
| | | | | | | | Library (Nanobody, | | PVVAWQMTWYRQAPGKEREWVAAIR | |
| | | | | | | | non-immune) | | SFGVSTHYADSVKGRFTISRDNAKNTVYL | |
| | | | | | | | | | QMNSLKPEDTAVYYCNVKDWGYEYEG | |
| | | | | | | | | | YDYWGQGTQVTVSS | |
| Sb61 | Nb | SARS-CoV2 | | | | S; RBD | Phage Display | 1112 | QVQLVESGGGLVQAGGSLRLSCAASGF | |
| | | | | | | | Library (Nanobody, | | PVQHEWMEWYRQAPGKEREWVAAIT | |
| | | | | | | | non-immune) | | SYGYRTYYADSVKGRFTISRDNAKNTVYL | |
| | | | | | | | | | QMNSLKPEDTAVYYCNVKDTGTYQAW | |
| | | | | | | | | | YDYWGQGTQVTVSS | |
| Sb62 | Nb | SARS-CoV2 | | SARS-CoV2 | | S; RBD | Phage Display | 1113 | QVQLVESGGGLVQAGGSLRLSCAASGF | |
| | | | | | | | Library (Nanobody, | | PVEQEHMYWYRQASGKEREWVAAIVS | |
| | | | | | | | non-immune) | | EGAYTAYADSVKGRFTISRDNAKNTVYL | |
| | | | | | | | | | QMNSLKPEDTAVYYCNVKDWGGYQW | |
| | | | | | | | | | YYDYWGQGTQVTVSS | |
| Sb63 | Nb | SARS-CoV2 | | | | S; RBD | Phage Display | 1114 | QVQLVESGGGLVQAGGSLRLSCAASGF | |
| | | | | | | | Library (Nanobody, | | PVNAEIMFWYRQAPGKEREWVAAIKSA | |
| | | | | | | | non-immune) | | GTTTLYADSVKGRFTISRDNAKNTVYLQ | |
| | | | | | | | | | MNSLKPEDTAVYYCNVKDYGAQAHYYD | |
| | | | | | | | | | YWGQGTQVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| Sb5 | N/A | IGHV3-3 | IGHJ4 | N/A | N/A | 3224 | YVYVGASY | | N/A | Tania Custodia et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb50 | N/A | IGHV3S5 | IGHJ4 | N/A | N/A | 3225 | NVKDWGWL | | N/A | Tania Custodia et al., |
| | | 3 | (Alpaca) | | | | AQYYDY | | | 2020 |
| | | (Alpaca) | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb52 | N/A | IGHV3S5 | IGHJ4 | N/A | N/A | 3226 | NVKDEGMW | | N/A | Tania Custodia et al., |
| | | 3 | (Alpaca) | | | | QHYYDY | | | 2020 |
| | | (Alpaca) | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb54 | N/A | IGHV3S5 | IGHJ4 | N/A | N/A | 3227 | NVKDEGNSQ | | N/A | Tania Custodia et al., |
| | | 3 | (Alpaca) | | | | SHYDY | | | 2020 |
| | | (Alpaca) | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb56 | N/A | IGHV3S5 | IGHJ4 | N/A | N/A | 3228 | NVKDAGNSK | | N/A | Tania Custodia et al., |
| | | 3 | (Alpaca) | | | | ALYDY | | | 2020 |
| | | (Alpaca) | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb57 | N/A | IGHV3S5 | IGHJ4 | N/A | N/A | 3229 | NVKDWGRA | | N/A | Tania Custodia et al., |
| | | 3 | (Alpaca) | | | | GARYDY | | | 2020 |
| | | (Alpaca) | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb58 | N/A | IGHV3S5 | IGHJ4 | N/A | N/A | 3230 | NVKDMDRW | | N/A | Tania Custodia et al., |
| | | 3 | (Alpaca) | | | | RTTYDY | | | 2020 |
| | | (Alpaca) | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb6 | N/A | IGHV3-3 | IGHJ4 | N/A | N/A | 3231 | YVWVGNQY | | N/A | Tania Custodia et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb60 | N/A | IGHV3S5 | IGHJ4 | N/A | N/A | 3232 | NVKDWGYEY | | N/A | Tania Custodia et al., |
| | | 3 | (Alpaca) | | | | EGYDY | | | 2020 |
| | | (Alpaca) | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb61 | N/A | IGHV3S5 | IGHJ4 | N/A | N/A | 3233 | NVKDTGTYQ | | N/A | Tania Custodia et al., |
| | | 3 | (Alpaca) | | | | AWYDY | | | 2020 |
| | | (Alpaca) | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb62 | N/A | IGHV3S5 | IGHJ4 | N/A | N/A | 3234 | NVKDWGGY | | N/A | Tania Custodia et al., |
| | | 3 | (Alpaca) | | | | QWYYDY | | | 2020 |
| | | (Alpaca) | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb63 | N/A | IGHV3S5 | IGHJ4 | N/A | N/A | 3235 | NVKDYGAQA | | N/A | Tania Custodia et al., |
| | | 3 | (Alpaca) | | | | HYYDY | | | 2020 |
| | | (Alpaca) | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| Sb67 | Nb | SARS-CoV2 | | | | S; RBD | Phage Display | 1115 | QVQLVESGGGLVQAGGSLRLSCAASGF | |
| | | | | | | | Library (Nanobody, | | PVMWAHMAWYRQAPGKEREWVAAI | |
| | | | | | | | non-immune) | | VSAGAYTHYADSVKGRFTISRDNAKNTV | |
| | | | | | | | | | YLQMNSLKPEDTAVYYCNVKDWGTYNS | |
| | | | | | | | | | YYDYWGQGTQVTVSS | |
| Sb7 | Nb | SARS-CoV2 | | SARS-CoV2 | | S; RBD | Phage Display | 1116 | QVQLVESGGGLVQAGGSLRLSCAASGF | |
| | | | | | | | Library (Nanobody, | | PVNAEEMEWYRQAPGKEREWVAAISS | |
| | | | | | | | non-immune) | | SGDWTYYADSVKGRFTISRDNAKNTVYL | |
| | | | | | | | | | QMNSLKPEDTAVYYCLVYVGSTYIGQGT | |
| | | | | | | | | | QVTVSS | |
| Sb71 | Nb | SARS-CoV2 | | | | S; RBD | Phage Display | 1117 | QVQLVESGGGSVQAGGSLRLSCAASGN | |
| | | | | | | | Library (Nanobody, | | IQHIKYLGWFRQAPGREREGVAALMITR | |
| | | | | | | | non-immune) | | YGQTYYADSVKGRFTVSLDNAKNTVYLQ | |
| | | | | | | | | | MNSLKPEDTALYYCAAAHYGDNFPLAY | |
| | | | | | | | | | QAYLYWGQGTQVTVSS | |
| Sb75 | Nb | SARS-CoV2 | | | | S; RBD | Phage Display | 1118 | QVQLVESGGGSVQAGGSLRLSCAASGYI | |
| | | | | | | | Library (Nanobody, | | NQIYYLGWFRQAPGKEREGVAALNTYQ | |
| | | | | | | | non-immune) | | GQTYYADSVKGRFTVSLDNAKNTVYLQ | |
| | | | | | | | | | MNSLKPEDTALYYCAAARWGRDEPLYH | |
| | | | | | | | | | YYYSYWGQGTQVTVSS | |
| Sb76 | Nb | SARS-CoV2 | | SARS-CoV2 | | S; RBD | Phage Display | 1119 | QVQLVESGGGLVQAGGSLRLSCAASSFP | |
| | | | | | | | Library (Nanobody, | | VDTYHMAWYRQAPGKEREWVAAIVS | |
| | | | | | | | non-immune) | | WGWRTYYADSVKGRFTISRDNAKNTVY | |
| | | | | | | | | | LQMNSLKPEDTAVYYCNVKDIGAQEVH | |
| | | | | | | | | | YDYWGQGTQVTVSS | |
| Sb78 | Nb | SARS-CoV2 | | | | S; RBD | Phage Display | 1120 | QVQLVESGGGSVQAGGSLRLSCAASGYI | |
| | | | | | | | Library (Nanobody, | | KSIKYLGWFRQAPGKEREGVAALMTRY | |
| | | | | | | | non-immune) | | GETYYADSVKGRFTVSLDNAKNTVYLQ | |
| | | | | | | | | | MNSLKPEDTALYYCAAANYGNNWPLTG | |
| | | | | | | | | | VNYWYWGQGTQVTVSS | |
| Sb8 | Nb | SARS-CoV2 | | | | S; RBD | Phage Display | 1121 | QVQLVESGGGLVQAGGSLRLSCAASGF | |
| | | | | | | | Library (Nanobody, | | PVKSYEMEWYRQAPGKEREWVAAISSY | |
| | | | | | | | non-immune) | | GEYTEYADSVKGRFTISRDNAKNTVYLQ | |
| | | | | | | | | | MNSLKPEDTAVYYCYVWVGDSYLGQGT | |
| | | | | | | | | | QVTVSS | |
| Sb83 | Nb | SARS-CoV2 | | | | S; RBD | Phage Display | 1122 | QVQLVESGGGSVQAGGSLRLSCAASGG | |
| | | | | | | | Library (Nanobody, | | ITHIVYLGWFRQAPGKEREGVAALMTR | |
| | | | | | | | non-immune) | | WGTTYYADSVKGRFTVSLDNAKNTVYL | |
| | | | | | | | | | QMNSLKPEDTALYYCAAAKYGQNFPLSY | |
| | | | | | | | | | HAYRYWGQGTQVTVSS | |
| Sb84 | Nb | SARS-CoV2 | | | | S; RBD | Phage Display | 1123 | QVQLVESGGGSVQAGGSLRLSCAASGYI | |
| | | | | | | | Library (Nanobody, | | KHIEYLGWFRQAPGKEREGVAALKTSSG | |
| | | | | | | | non-immune) | | STYYADSVKGRFTVSLDNAKNTVYLQM | |
| | | | | | | | | | NSLKPEDTALYYCAAARYGRSDPLHYHE | |
| | | | | | | | | | YSYWGQGTQVTVSS | |
| Sb85 | Nb | SARS-CoV2 | | | | S; RBD | Phage Display | 1124 | QVQLVESGGGSVQAGGSLRLSCAASGSI | |
| | | | | | | | Library (Nanobody, | | SSITYLGWFRQAPGKEREGVAALVTSRG | |
| | | | | | | | non-immune) | | KTYYADSVKGRFTVSLDNAKNTVYLQM | |
| | | | | | | | | | NSLKPEDTALYYCAAASWGYTWPLYTYD | |
| | | | | | | | | | YWYWGQGTQVTVSS | |
| Sb88 | Nb | SARS-CoV2 | | | | S; RBD | Phage Display | 1125 | QVQLVESGGGLVQAGGSLRLSCAASGF | |
| | | | | | | | Library (Nanobody, | | PVWAHHMLWYRQAPGKEREWVAAIA | |
| | | | | | | | non-immune) | | SWGANTAYADSVKGRFTISRDNAKNTV | |
| | | | | | | | | | YLQMNSLKPEDTAVYYCNVKDSGQYRE | |
| | | | | | | | | | NYDYWGQGTQVTVSS | |
| Sb9 | Nb | SARS-CoV2 | | | | S; RBD | Phage Display | 1126 | QVQLVESGGGLVQAGGSLRLSCAASGF | |
| | | | | | | | Library (Nanobody, | | PVGQQHMYWYRQAPGKEREWVAAIYS | |
| | | | | | | | non-immune) | | YGHITKYADSVKGRFTISRDNAKNTVYLQ | |
| | | | | | | | | | MNSLKPEDTAVYYCWVYVGDYYEGQG | |
| | | | | | | | | | TQVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| Sb67 | N/A | IGHV3S5 | IGHJ4 | N/A | N/A | 3236 | NVKDWGTYN | | N/A | Tania Custodia et al., |
| | | 3 | (Alpaca) | | | | SYYDY | | | 2020 |
| | | (Alpaca) | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb7 | N/A | IGHV3-3 | IGHJ4 | N/A | N/A | 3237 | LVYVGSTY | | N/A | Tania Custodia et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb71 | N/A | IGHV3-3 | IGHJ4 | N/A | N/A | 3238 | AAAHYGDNF | | N/A | Tania Custodia et al., |
| | | (Alpaca) | (Alpaca) | | | | PLAYQAYLY | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb75 | N/A | IGHV3-3 | IGHJ4 | N/A | N/A | 3239 | AAARWGRDE | | N/A | Tania Custodia et al., |
| | | (Alpaca) | (Alpaca) | | | | PLYHYYYSY | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb76 | N/A | IGHV3-3 | IGHJ4 | N/A | N/A | 3240 | NVKDIGAQEV | | N/A | Tania Custodia et al., |
| | | (Alpaca) | (Alpaca) | | | | HYDY | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb78 | N/A | IGHV3-3 | IGHJ4 | N/A | N/A | 3241 | AAANYGNN | | N/A | Tania Custodia et al., |
| | | (Alpaca) | (Alpaca) | | | | WPLTGVNYW | | | 2020 |
| | | | | | | | Y | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb8 | N/A | IGHV3-3 | IGHJ4 | N/A | N/A | 3242 | YVWVGDSY | | N/A | Tania Custodia et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb83 | N/A | IGHV3-3 | IGHJ4 | N/A | N/A | 3243 | AAAKYGQNF | | N/A | Tania Custodia et al., |
| | | (Alpaca) | (Alpaca) | | | | PLSYHAYRY | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb84 | N/A | IGHV3-3 | IGHJ4 | N/A | N/A | 3244 | AAARYGRSDP | | N/A | Tania Custodia et al., |
| | | (Alpaca) | (Alpaca) | | | | LHYHEYSY | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb85 | N/A | IGHV3-3 | IGHJ4 | N/A | N/A | 3245 | AAASWGYTW | | N/A | Tania Custodia et al., |
| | | (Alpaca) | (Alpaca) | | | | PLYTYDYWY | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb88 | N/A | IGHV3S5 | IGHJ4 | N/A | N/A | 3246 | NVKDSGQYR | | N/A | Tania Custodia et al., |
| | | 3 | (Alpaca) | | | | ENYDY | | | 2020 |
| | | (Alpaca) | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb9 | N/A | IGHV3-3 | IGHJ4 | N/A | N/A | 3247 | WVYVGDYY | | N/A | Tania Custodia et al., |
| | | (Alpaca) | (Alpaca) | | | | | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| Sb90 | Nb | SARS-CoV2 | | | | S; RBD | Phage Display | 1127 | QVQLVESGGGSVQAGGSLRLSCAASGA | |
| | | | | | | | Library (Nanobody, | | INQIYYLGWFRQAPGKEREGVAALSTKY | |
| | | | | | | | non-immune) | | GETYYADSVKGRFTVSLDNAKNTVYLQ | |
| | | | | | | | | | MNSLKPEDTALYYCAAARWGRQYPLTF | |
| | | | | | | | | | VYYSYWGQGTQVTVSS | |
| Sb93 | Nb | SARS-CoV2 | | | | S; RBD | Phage Display | 1128 | QVQLVESGGGSVQAGGSLRLSCAASGH | |
| | | | | | | | Library (Nanobody, | | IAQIEYLGWFRQAPGKEREGVAALSTNQ | |
| | | | | | | | non-immune) | | GYTYYADSVKGRFTVSLDNAKNTVYLQ | |
| | | | | | | | | | MNSLKPEDTALYYCAAARWGRTYPLSY | |
| | | | | | | | | | MAYTYWGQGTQVTVSS | |
| Sb94 | Nb | SARS-CoV2 | | | | S; RBD | Phage Display | 1129 | QVQLVESGGGSVQAGGSLRLSCAASGYI | |
| | | | | | | | Library (Nanobody, | | TMIEYLGWFRQAPGKEREGVAALNTHT | |
| | | | | | | | non-immune) | | GGTYYADSVKGRFTVSLDNAKNTVYLQ | |
| | | | | | | | | | MNSLKPEDTALYYCAAARWGRYEPLHY | |
| | | | | | | | | | AYYSYWGQGTQVTVSS | |
| Sb95 | Nb | SARS-CoV2 | | | | S; RBD | Phage Display | 1130 | QVQLVESGGGSVQAGGSLRLSCAASGN | |
| | | | | | | | Library (Nanobody, | | IYNIKYLGWFRQAPGKEREGVAALMTRY | |
| | | | | | | | non-immune) | | GETYYADSVKGRFTVSLDNAKNTVYLQ | |
| | | | | | | | | | MNSLKPEDTALYYCAAASYGANWPLVS | |
| | | | | | | | | | AAYTYWGQGTQVTVSS | |
| Sb97 | Nb | SARS-CoV2 | | | | S; RBD | Phage Display | 1131 | QVQLVESGGGSVQAGGSLRLSCAASGA | |
| | | | | | | | Library (Nanobody, | | ISTIEYLGWFRQAPGREREGVAALYTER | |
| | | | | | | | non-immune) | | GYTYYADSVKGRFTVSLDNAKNTVYLQ | |
| | | | | | | | | | MNSLKPEDTALYYCAAARYGHAQAPLH | |
| | | | | | | | | | YFWYGYWGQGTQVTVSS | |
| SR1 | Nb | SARS-CoV2 | | | | S; RBD | Phage Display | 1132 | QVQLVESGGGLVQAGGSLRLSCAASGF | |
| | | | | | | | Library (Nanobody, | | PVAAYEMEWYRQAPGKEREWVAAINS | |
| | | | | | | | non-immune) | | MGDQTYYADSVKGRFTISRDNAKNTVY | |
| | | | | | | | | | LQMNSLKPEDTAVYYCYVYVGFSYIGQG | |
| | | | | | | | | | TQVTVSS | |
| SR13 | Nb | SARS-CoV2 | | | | S; RBD | Phage Display | 1133 | QVQLVESGGGLVQAGGSLRLSCAASGF | |
| | | | | | | | Library (Nanobody, | | PVDYMEMEWFRQAPGKEREWVAAITS | |
| | | | | | | | non-immune) | | NGRETYYADSVKGRFTVSRDNAKNTVYL | |
| | | | | | | | | | QMNSLKPEDTAVYYCYVYVGSSYIGQGT | |
| | | | | | | | | | QVTVSS | |
| SR18 | Nb | SARS-CoV2 | | | | S; RBD | Phage Display | 1134 | QVQLVESGGGLVQAGGSLRLSCAASGF | |
| | | | | | | | Library (Nanobody, | | PVWFQEMEWYRQAPGKEREWVAAISS | |
| | | | | | | | non-immune) | | QGTHTYYADSVKGRFTISRDNAKNTVYL | |
| | | | | | | | | | QMNSLKPEDTAVYYCYVYVGASYLGQG | |
| | | | | | | | | | TQVTVSS | |
| SR31 | Nb | SARS-CoV2 | | | | S; RBD | Phage Display | 1135 | QVQLVESGGGLVQAGGSLRLSCAASGF | |
| | | | | | | | Library (Nanobody, | | PVWQGEMAWYRQAPGKEREWVAAIS | |
| | | | | | | | non-immune) | | SMGYKTYYADSVKGRFTISRDNAKNTVY | |
| | | | | | | | | | LQMNSLKPEDTAVYYCAVMVGFWYAG | |
| | | | | | | | | | QGTQVTVSS | |
| SR34 | Nb | SARS-CoV2 | | | | S; RBD | Phage Display | 1136 | QVQLVESGGGLVQAGGSLRLSCAAGGF | |
| | | | | | | | Library (Nanobody, | | PVKDHEMEWYRQAPGKEREWVAAITS | |
| | | | | | | | non-immune) | | SGWGTNTYYADSVKGRFTISRDNAKNT | |
| | | | | | | | | | VYLQMNSLKPEDTAVYYCYVYVGSSYIG | |
| | | | | | | | | | QGTQVTVSS | |
| SR38 | Nb | SARS-CoV2 | | | | S; RBD | Phage Display | 1137 | QVQLVESGGGLVQAGGSLRLSCAASGF | |
| | | | | | | | Library (Nanobody, | | PVNQEEMEWYRQAPGKEREWVAAIKS | |
| | | | | | | | non-immune) | | WGTLTAYADSVKGRFTISRDNAKNTVYL | |
| | | | | | | | | | QMNSLKPEDTAVYYCAVHVGQTYIGQG | |
| | | | | | | | | | TQVTVSS | |
| SR4 | Nb | SARS-CoV2 | | | | S; RBD | Phage Display | 1138 | QVQLVESGGGLVQAGGSLRLSCAASGF | |
| | | | | | | | Library (Nanobody, | | PVYSWNMWWYRQAPGKEREWVAAIE | |
| | | | | | | | non-immune) | | SHGDSTRYADSVKGRFTISRDNAKNTVY | |
| | | | | | | | | | LQMNSLKPEDTAVYYCYVWVGHTYYG | |
| | | | | | | | | | QGTQVTVSS | |
| SR5 | Nb | SARS-CoV2 | | | | S; RBD | Phage Display | 1139 | QVQLVESGGGLVQAGGSLRLRCAASGF | |
| | | | | | | | Library (Nanobody, | | PVETTEMEWYRQAPGKEREWVAAISSY | |
| | | | | | | | non-immune) | | GSETYYADSVKGRFTISRDNAKNTVYLQ | |
| | | | | | | | | | MNSLKPEDTAVYYCYVYVGTSYLGQGT | |
| | | | | | | | | | QVTVSS | |
| SR7 | Nb | SARS-CoV2 | | | | S; RBD | Phage Display | 1140 | QVQLVESGGGLVQAGGSLRLSCAASGF | |
| | | | | | | | Library (Nanobody, | | PVWSNEMEWYRQAPGKEREWVAAITS | |
| | | | | | | | non-immune) | | YGTTEYADSVKGRFTISRDNAKNTVYLQ | |
| | | | | | | | | | MNSLKPEDTAVYYCYVYVGYSYIGQGTQ | |
| | | | | | | | | | VTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| Sb90 | N/A | IGHV3-3 | IGHJ4 | N/A | N/A | 3248 | AAARWGRQY | | N/A | Tania Custodia et al., |
| | | (Alpaca) | (Alpaca) | | | | PLTFVYYSY | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb93 | N/A | IGHV3-3 | IGHJ4 | N/A | N/A | 3249 | AAARWGRTY | | N/A | Tania Custodia et al., |
| | | (Alpaca) | (Alpaca) | | | | PLSYMAYTY | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb94 | N/A | IGHV3-3 | IGHJ4 | N/A | N/A | 3250 | AAARWGRYE | | N/A | Tania Custodia et al., |
| | | (Alpaca) | (Alpaca) | | | | PLHYAYYSY | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb95 | N/A | IGHV3-3 | IGHJ4 | N/A | N/A | 3251 | AAASYGANW | | N/A | Tania Custodia et al., |
| | | (Alpaca) | (Alpaca) | | | | PLVSAAYTY | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| Sb97 | N/A | IGHV3-3 | IGHJ4 | N/A | N/A | 3252 | AAARYGHAQ | | N/A | Tania Custodia et al., |
| | | (Alpaca) | (Alpaca) | | | | APLHYFWYGY | | | 2020 |
| | | | | | | | | | | (https://www.biorxiv.or |
| | | | | | | | | | | g/content/10.1101/202 |
| | | | | | | | | | | 0.06.23.165415v1.full.p |
| | | | | | | | | | | df+html) |
| SR1 | N/A | IGHV3-3 | IGHJ4 | N/A | N/A | 3253 | YVYVGFSY | | N/A | Tingting Li et al., 2020 |
| | | (Alpaca) | (Alpaca) | | | | | | | (https://www.biorxiv.or |
| | | | | | | | | | | g/content/10.1101/202 |
| | | | | | | | | | | 0.06.09.143438v1) |
| SR13 | N/A | IGHV3-3 | IGHJ4 | N/A | N/A | 3254 | YVYVGSSY | | N/A | Tingting Li et al., 2020 |
| | | (Alpaca) | (Alpaca) | | | | | | | (https://www.biorxiv.or |
| | | | | | | | | | | g/content/10.1101/202 |
| | | | | | | | | | | 0.06.09.143438v1) |
| SR18 | N/A | IGHV3-3 | IGHJ4 | N/A | N/A | 3255 | YVYVGASY | | N/A | Tingting Li et al., 2020 |
| | | (Alpaca) | (Alpaca) | | | | | | | (https://www.biorxiv.or |
| | | | | | | | | | | g/content/10.1101/202 |
| | | | | | | | | | | 0.06.09.143438v1) |
| SR31 | N/A | IGHV3-3 | IGHJ4 | N/A | N/A | 3256 | AVMVGFWY | | N/A | Tingting Li et al., 2020 |
| | | (Alpaca) | (Alpaca) | | | | | | | (https://www.biorxiv.or |
| | | | | | | | | | | g/content/10.1101/202 |
| | | | | | | | | | | 0.06.09.143438v1) |
| SR34 | N/A | IGHV3-3 | IGHJ4 | N/A | N/A | 3257 | YVYVGSSY | | N/A | Tingting Li et al., 2020 |
| | | (Alpaca) | (Alpaca) | | | | | | | (https://www.biorxiv.or |
| | | | | | | | | | | g/content/10.1101/202 |
| | | | | | | | | | | 0.06.09.143438v1) |
| SR38 | N/A | IGHV3-3 | IGHJ4 | N/A | N/A | 3258 | AVHVGQTY | | N/A | Tingting Li et al., 2020 |
| | | (Alpaca) | (Alpaca) | | | | | | | (https://www.biorxiv.or |
| | | | | | | | | | | g/content/10.1101/202 |
| | | | | | | | | | | 0.06.09.143438v1) |
| SR4 | N/A | IGHV3-3 | IGHJ4 | N/A | N/A | 3259 | YVWVGHTY | | N/A | Tingting Li et al., 2020 |
| | | (Alpaca) | (Alpaca) | | | | | | | (https://www.biorxiv.or |
| | | | | | | | | | | g/content/10.1101/202 |
| | | | | | | | | | | 0.06.09.143438v1) |
| SR5 | N/A | IGHV3-3 | IGHJ4 | N/A | N/A | 3260 | YVYVGTSY | | N/A | Tingting Li et al., 2020 |
| | | (Alpaca) | (Alpaca) | | | | | | | (https://www.biorxiv.or |
| | | | | | | | | | | g/content/10.1101/202 |
| | | | | | | | | | | 0.06.09.143438v1) |
| SR7 | N/A | IGHV3S5 | IGHJ4 | N/A | N/A | 3261 | YVYVGYSY | | N/A | Tingting Li et al., 2020 |
| | | 3 | (Alpaca) | | | | | | | (https://www.biorxiv.or |
| | | (Alpaca) | | | | | | | | g/content/10.1101/202 |
| | | | | | | | | | | 0.06.09.143438v1) |

| | | | | | Not | | | | | |
|---|---|---|---|---|---|---|---|---|---|---|
| | Ab | | | Neutral- | Neutral- | | | SEQ | | SEQ |
| | or | | Doesn't | ising | ising | Protein + | | ID | | ID |
| Name | Nb | Binds to | Bind to | Vs | Vs | Epitope | Origin | NO.: | VH or VHH | NO.: |
| Ty1 | Nb | SARS-CoV2 | | SARS-CoV2 | | S; RBD | Immunised Alpaca | 1141 | QVQLVETGGGLVQPGGSLRLSCAASGF | |
| | | | | | | | | | TFSSVYMNWVRQAPGKGPEWVSRISP | |
| | | | | | | | | | NSGNIGYTDSVKGRFTISRDNAKNTLYL | |
| | | | | | | | | | QMNNLKPEDTALYYCAIGLNLSSSSVRG | |
| | | | | | | | | | QGTQVTVSS | |
| VH-Fc- | Nb | SARS-CoV2 | | SARS-CoV2 | | S; RBD | Phage Display | | ND | |
| B01 | | | | | | | (Human) | | | |
| VH2-A01. | Nb | SARS-CoV2 | | SARS-CoV2 | | S; RBD | Phage Display | | ND | |
| B01 | | | | | | | (Human) | | | |
| VH2-A01- | Nb | SARS-CoV2 | | SARS-CoV2 | | S; RBD | Phage Display | | ND | |
| B02 | | | | | | | (Human) | | | |
| VH3-B01 | Nb | SARS-CoV2 | | SARS-CoV2 | | S; RBD | Phage Display | | ND | |
| | | | | | | | (Human) | | | |
| VHH-72 | Nb | SARS-CoV1, | | SARS-CoV2 | | S; RBD | Immunised Llama | 1142 | QVQLQESGGGLVQAGGSLRLSCAASGR | |
| | | SARS-CoV2 | | and SARS- | | | | | TFSEYAMGWFRQAPGKEREFVATISWS | |
| | | | | CoV1 | | | | | GGSTYYTDSVKGRFTISRDNAKNTVYLQ | |
| | | | | | | | | | MNSLKPDDTAVYYCAAAGLGTVVSEW | |
| | | | | | | | | | DYDYDYWGQGTQVTVSS | |
| W23UACh | Nb | SARS-CoV2 | | | | S; RBD | Immunised Alpaca | 1143 | QVQLVESGGGLVQPGESLRLSCAASGNI | |
| | | | | | | | | | FGIAAVHWFRKAPGKEREFTAGFGSDG | |
| | | | | | | | | | STNYANSVKGRFTISRDNAKNTTYLQMN | |
| | | | | | | | | | SLKPEDTAVYYCHALIKNELGFLDYWGP | |
| | | | | | | | | | GTQVTVSS | |
| W25UACh | Nb | SARS-CoV2 | | | | S; RBD | Immunised Alpaca | 1144 | QVQLVESGGGLVQPGESLRLSCAASGSI | |
| | | | | | | | | | FGIYAVHWFRMAPGKEREFTAGFGSHG | |
| | | | | | | | | | STNYAASVKGRFTMSRDNAKNTTYLQM | |
| | | | | | | | | | NSLKPADTAVYYCHALIKNELGFLDYWG | |
| | | | | | | | | | PGTQVTVSS | |

| | | | | | | SEQ | | SEQ | | |
|---|---|---|---|---|---|---|---|---|---|---|
| | | Heavy V | Heavy J | Light V | Light J | ID | | ID | | |
| Name | VL | Gene | Gene | Gene | Gene | NO.: | CDRH3 | NO.: | CDRL3 | Sources |
| Ty1 | N/A | IGHV3S1 | IGHJ4 | N/A | N/A | 3262 | AIGLNLSSSSV | | N/A | Leo Hanke et al., 2020 |
| | | (Alpaca) | (Alpaca) | | | | | | | (https://www.biorxiv.or |
| | | | | | | | | | | g/content/10.1101/202 |
| | | | | | | | | | | 0.06.02.130161v1) |
| VH-Fc- | ND | ND | ND | ND | ND | | ND | | ND | Colton J. Bracken et al., |
| B01 | | | | | | | | | | 2020 |
| | | | | | | | | | | (https://www.biorxiv.or |
| | | | | | | | | | | g/content/10.1101/202 |
| | | | | | | | | | | 0.08.08.242511v1.full.p |
| | | | | | | | | | | df) |
| VH2-A01. | ND | ND | ND | ND | ND | | ND | | ND | Colton J. Bracken et al., |
| B01 | | | | | | | | | | 2020 |
| | | | | | | | | | | (https://www.biorxiv.or |
| | | | | | | | | | | g/content/10.1101/202 |
| | | | | | | | | | | 0.08.08.242511v1.full.p |
| | | | | | | | | | | df) |
| VH2-A01- | ND | ND | ND | ND | ND | | ND | | ND | Colton J. Bracken et al., |
| B02 | | | | | | | | | | 2020 |
| | | | | | | | | | | (https://www.biorxiv.or |
| | | | | | | | | | | g/content/10.1101/202 |
| | | | | | | | | | | 0.08.08.242511v1.full.p |
| | | | | | | | | | | df) |
| VH3-B01 | ND | ND | ND | ND | ND | | ND | | ND | Colton J. Bracken et al., |
| | | | | | | | | | | 2020 |
| | | | | | | | | | | (https://www.biorxiv.or |
| | | | | | | | | | | g/content/10.1101/202 |
| | | | | | | | | | | 0.08.08.242511v1.full.p |
| | | | | | | | | | | df) |
| VHH-72 | N/A | IGHV3-3 | IGHJ4 | N/A | N/A | 3263 | AAAGLGTVVS | | N/A | Daniel Wrapp et al., |
| | | (Alpaca) | (Alpaca) | | | | EWDYDYDY | | | 2020 |
| | | | | | | | | | | (https://www.sciencedi |
| | | | | | | | | | | rect.com/science/articl |
| | | | | | | | | | | e/pii/S00928674203049 |
| | | | | | | | | | | 43) |
| W23UACh | N/A | IGHV3S5 | IGHJ4 | N/A | N/A | 3264 | HALIKNELGFL | | N/A | Guillermo Nieto et al., |
| | | 3 | (Alpaca) | | | | DY | | | 2020 |
| | | (Alpaca) | | | | | | | | (https://www.biorxiv.or |
| | | | | | | | | | | g/content/10.1101/202 |
| | | | | | | | | | | 0.06.09.137935v1) |
| W25UACh | N/A | IGHV3S5 | IGHJ4 | N/A | N/A | 3265 | HALIKNELGFL | | N/A | Guillermo Nieto et al., |
| | | 3 | (Alpaca) | | | | DY | | | 2020 |
| | | (Alpaca) | | | | | | | | (https://www.biorxiv.or |
| | | | | | | | | | | g/content/10.1101/202 |
| | | | | | | | | | | 0.06.09.137935v1) |

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A method comprising:
(a) administering a first composition to a subject, wherein said first composition comprises polycationic structures, and wherein said first composition is free, or essentially free, of nucleic acid molecules; and
(b) administering a second composition to said subject after administering said first composition, wherein said second composition comprises a plurality of one or more non-viral expression vectors that encode at least one anti-SARS-CoV-2 antibody or antigen-binding portion thereof, and/or recombinant ACE2, and
wherein, as a result of said administering said first and second compositions, said at least one anti-SARS-CoV-2 antibody, or antigen-binding portion thereof, and/or said recombinant ACE2, is expressed in said subject.

2. The method of claim 1, wherein:
a) said subject is infected with the SARS-CoV-2 virus, and wherein said at least one anti-SARS-CoV-2 antibody, or antigen-binding portion thereof, or recombinant ACE2 is expressed in said subject at an expression level sufficient to reduce: i) the SARS-CoV-2 viral load in said subject, and/or ii) at least one symptom in said subject caused by said SARS-CoV-2 infection; or
b) said subject is not infected with the SARS-CoV-2 virus, and wherein said at least one anti-SARS-CoV-2 antibody, or antigen-binding portion thereof, or recombinant ACE2 is expressed in said subject at an expression level sufficient to prevent said subject from being infected by the SARS-CoV-2 virus.

3. The method of claim 2, wherein said expression level is maintained in said subject for at least two weeks, at least one month, or at least one year without: i) any further, or only one, two, or three further repeat, of steps a) and b), and ii) any further administration of vectors encoding: said at least one anti-SARS-CoV-2 antibody or antigen-binding portion thereof, or said ACE2.

4. (canceled)

5. (canceled)

6. The method of claim 1, wherein said at least one anti-SARS-CoV-2 antibody, or antigen-binding portion thereof, is expressed in said subject at a level of: i) between 500 ng/ml and 50 ug/ml, or 10-20 ug/ml, for at least 25 days, or ii) at least 250 ng/ml for at least 25 days.

7. The method of claim 1, wherein said polycationic structures comprise cationic lipids.

8. The method of claim 7, wherein said first composition comprises a plurality of liposomes, wherein at least some of said liposomes comprise said cationic lipids.

9. The method of claim 8, wherein at least some of said liposomes comprise neutral lipids.

10. The method of claim 9, wherein the ratio of said cationic lipids to said neutral lipids in said liposomes is 95:05-80:20 or about 1:1.

11. The method of claim 10, wherein said cationic and neutral lipids are selected from the group consisting of: distearoyl phosphatidyl choline (DSPC); hydrogenated or non-hydrogenated soya phosphatidylcholine (HSPC); distearoylphosphatidylethanolamine (DSPE); egg phosphatidylcholine (EPC); 1,2-Distearoyl-sn-glycero-3-phospho-rac-glycerol (DSPG); dimyristoyl phosphatidylcholine (DMPC); 1,2-Dimyristoyl-sn-glycero-3-phosphoglycerol (DMPG); 1,2-Dipalmitoyl-sn-glycero-3-phosphate (DPPA); trimethylammonium propane lipids; DOTIM (1-[2-9(2)-octadecenoylloxy)ethyl]-2-(8(2)-heptadecenyl)-3-(2-hydroxyethyl) midizolinium chloride) lipids; and mixtures of two or more thereof.

12. (canceled)

13. (canceled)

14. The method of claim 1, wherein: A) said antigen-binding portion thereof is selected from the group consisting of: a Fab′, F(ab)2, Fab, and a minibody, or B) wherein said at least one anti-SARS-CoV-2 antibody, or antigen-binding portion thereof, is bi-specific for different SARS-CoV-2 antigens.

15. (canceled)

16. The method of claim 1, wherein said anti-SARS-CoV-2 antibody, or antigen-binding portion thereof, comprises one, two, three, four, five, six, seven, eight, nine, ten, eleven, twelve, or more antibodies selected from: REGN10933, REGN10987; VIR-7831; LY-CoV1404; LY3853113; Zost 2355K; CV07-209K; C121L; Zost 2504L; CV38-183L; COVA215K; RBD215; CV07-250L; C144L; COVA118L; C135K; and B38.

17. (canceled)

18. The method of claim 1, wherein said at least one anti-SARS-CoV-2 antibody, or antigen-binding portion thereof, comprises at least two, or at least four, or at least eight, or at least 11 anti-SARS-CoV-2 antibodies, or antigen-binding portions thereof, which are expressed in said subject at an expression level sufficient to reduce: i) the SARS-CoV-2 viral load in said subject, or ii) at least one symptom in said subject caused by said SARS-CoV-2 infection.

19. (canceled)

20. (canceled)

21. The method of claim 1,
(a) wherein said administering comprises intravenous administering; or
(b) wherein said second composition is administered: i) between 0.5 and 80 minutes after said first composition, or between about 1 and 20 minutes after said first composition.

22. (canceled)

23. The method of claim 1, further comprising: c) administering an agent, in said first or second composition, or present in a third composition, wherein said agent: i) increases the level of expression of said at least one anti-SARS-CoV-2 antibody or antigen-binding portion thereof, or ii) increases the length of time of said expression compared to when said agent is not administered to said subject.

24. The method of claim 23, wherein
(a) said agent is present in said first composition,
(b) said agent is present in said third composition, and is administered at least one hour prior to said first composition:
(c) said agent is a dexamethasone fatty acid ester:
(d) said agent is dexamethasone fatty acid ester having the following Formula: 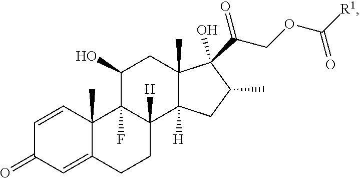
wherein R¹ is C₅-C₂₃ alkyl or C₅-C₂₃ alkenyl; or
(e) wherein said agent is present in said first, second, or third composition at a concentration of 0.01-35 mg/ml.

25.-28. (canceled)

29. The method of claim 1,
(a) wherein said subject has lung, cardiovascular, and/or multi-organ inflammation,
(b) wherein said subject is on a ventilator: or
(c) wherein said subject is a human.

30. (canceled)

31. The method of claim 1,
(a) wherein said first or second compositions further comprise a physiologically tolerable buffer or intravenous solution; or
(b) wherein said first or second compositions further comprise lactated Ringer's solution or saline solution.

32. (canceled)

33. The method of claim 1, wherein said first compositions comprise liposomes comprising said polycationic structures, wherein said liposomes further comprising one or more macrophage targeting moieties selected from the group consisting of: mannose moieties, maleimide moieties, a folate receptor ligand, folate, folate receptor antibody or fragment thereof, formyl peptide receptor ligands, N-formyl-Met-Leu-Phe, tetrapeptide Thr-Lys-Pro-Arg, galactose, and lactobionic acid.

34. The method of claim 1,
(a) wherein said one or more non-viral expression vectors comprise plasmids;
(b) wherein said plurality of one or more non-viral expression vectors are not attached to, or encapsulated in, any delivery agent;
(c) wherein 0.05-60 mg/mL of said expression vectors are present in said second composition; or
(d) wherein said polycationic structures comprise cationic liposomes which are present at a concentration of 0.5-100 mM in said first composition.

35.-37. (canceled)

38. The method of claim 1, wherein said subject is a human, wherein:
i) an amount of said first composition is administered such that said human receives a dosage of 2-50 mg/kg of said polycationic structures; and/or
ii) an amount of said second composition is administered such that said human receives a dosage of 0.05-60 mg/kg of said expression vectors.

39. The method of claim 1, wherein said polycationic structures comprise cationic liposomes, wherein said cationic liposomes further comprise a lipid bi-layer integrating peptide and/or a target peptide.

40. The method of claim 39, wherein: i) said lipid bi-layer integrating peptide is selected from the group consisting of: surfactant protein D (SPD), surfactant protein C (SPC), surfactant protein B (SPB), and surfactant protein A (SPA), and ii) said target peptide is selected from the group consisting of: microtubule-associated sequence (MTAS), nuclear localization signal (NLS), ER secretion peptide, ER retention peptide, and peroxisome peptide.

41. The method of claim 1, wherein steps a) and b) are repeated between 1 and 60 days after the initial step b).

42. The method of claim 1, wherein each of said non-viral expression vectors comprise between 5,500 and 30,000 nucleic acid base pairs.

43. The method of claim 1, further comprising: administering an anti-viral agent, an anti-inflammatory, or anticoagulant to said subject.

44. The method of claim 43, wherein said anti-viral agent comprises Remdesivir or a protein comprising at least part of the ACE2 receptor.

45. (canceled)

46. The method of claim 1, wherein said one or more non-viral expression vectors are CPG-free or CPG-reduced.

47. A system comprising:
a) a first container;
b) a first composition inside said first container and comprising polycationic structures, wherein said first composition is free, or essentially free, of nucleic acid molecules;
c) a second container; and
d) a second composition inside said second container and comprising a plurality of one or more non-viral expression vectors that encode
(i) at least one anti-SARS-CoV-2 antibody or antigen-binding portion thereof, or an ACE2 protein; or
(ii) at least three different antibodies or antigen-binding portions thereof.

48.-51. (canceled)

52. A method of simultaneously expressing at least three different antibodies, or antigen binding portions thereof, in a subject comprising:
a) administering a first composition to a subject, wherein said first composition comprises polycationic structures, and wherein said first composition is free, or essentially free, of nucleic acid molecules; and
b) administering a second composition to said subject after administering said first composition, wherein said second composition comprises a plurality of one or more non-viral expression vectors that encode at least three different antibodies or antigen-binding portions thereof, and
wherein, as a result of said administering said first and second compositions, said at least three different antibodies, or antigen-binding portions thereof, are simultaneously expressed in said subject.

53.-103. (canceled)

104. A method comprising:
a) administering a first composition to a subject, wherein said first composition comprises polycationic structures, and wherein said first composition is free, or essentially free, of nucleic acid molecules; and
b) administering a second composition to said subject after administering said first composition, wherein said second composition comprises a plurality of non-viral expression vectors that encode human growth hormone (hGH) and/or hGH linked to a half-life extending peptide (hGH-ext), and
wherein, as a result of said administering said first and second compositions, said hGH is expressed in said subject.

105.-139. (canceled)
